(19) United States
(12) Patent Application Publication
(10) Pub. No.: US 2025/0228130 A1
(43) Pub. Date: Jul. 10, 2025

(54) LIGHT-EMITTING ELEMENT, AMINE COMPOUND FOR THE LIGHT-EMITTING ELEMENT, AND DISPLAY DEVICE INCLUDING THE LIGHT-EMITTING ELEMENT

(71) Applicant: Samsung Display Co., Ltd., Yongin-si (KR)

(72) Inventors: XiuLan JIN, Yokohama (JP); Naoya SAKAMOTO, Yokohama (JP); Ichinori TAKADA, Yokohama (JP)

(73) Assignee: Samsung Display Co., Ltd., Yongin-si (KR)

(21) Appl. No.: 18/947,140

(22) Filed: Nov. 14, 2024

(30) Foreign Application Priority Data
Jan. 10, 2024 (KR) 10-2024-0004046

(51) Int. Cl. H10K 85/60; C07D 307/91; C07D 333/76; C07D 405/12; H10K 50/15

(52) U.S. Cl. CPC H10K 85/636; C07D 307/91; C07D 333/76; C07D 405/12; H10K 85/633; H10K 50/156; H10K 85/615; H10K 85/626; H10K 85/6572; H10K 85/6574; H10K 85/6576

(57) ABSTRACT

Embodiments provide an amine compound, a light-emitting element including the amine compound, and a display device including the light-emitting element. The light-emitting element includes a first electrode, a hole transport region disposed on the first electrode, an emission layer disposed on the hole transport region, an electron transport region disposed on the emission layer, and a second electrode disposed on the electron transport region, wherein the hole transport region includes the amine compound. The amine compound is represented by Formula 1, which is explained in the specification:

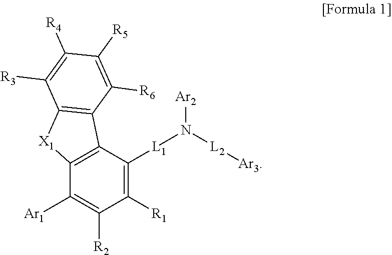

### CROSS-REFERENCE TO RELATED APPLICATION(S)

This application claims priority to and benefits of Korean Patent Application No. 10-2024-0004046 under 35 U.S.C. § 119, filed on Jan. 10, 2024 in the Korean Intellectual Property Office, the entire contents of which are incorporated herein by reference.

### BACKGROUND

### 1. Technical Field

The disclosure relates to a light-emitting element, an amine compound used in the light-emitting element, and a display device including the light-emitting element.

### 2. Description of the Related Art

Ongoing development continues for organic electroluminescence display devices and the like as image display devices. Organic electroluminescence display devices and the like are display devices that include a so-called self-luminous light-emitting element wherein holes and electrons are respectively injected from a first electrode and a second electrode recombine in an emission layer, so that a light-emitting material of the emission layer emits light to achieve display.

In the application of a light-emitting element to display devices, there is a persistent demand for improvements in luminous efficiency, lifespan, and the like. Continuous development is required on materials for a light-emitting element that are capable of stably achieving such characteristics.

It is to be understood that this background of the technology section is, in part, intended to provide useful background for understanding the technology. However, this background of the technology section may also include ideas, concepts, or recognitions that were not part of what was known or appreciated by those skilled in the pertinent art prior to a corresponding effective filing date of the subject matter disclosed herein.

### SUMMARY

The disclosure provides a light-emitting element having improved luminous efficiency and lifespan and a display device including the same.

The disclosure also provides an amine compound as a material for the light-emitting element, which improves the luminous efficiency and lifespan.

According to an embodiment, a light-emitting element may include a first electrode, a hole transport region disposed on the first electrode, an emission layer disposed on the hole transport region, an electron transport region disposed on the emission layer, and a second electrode disposed on the electron transport region, wherein the hole transport region may include an amine compound represented by Formula 1:

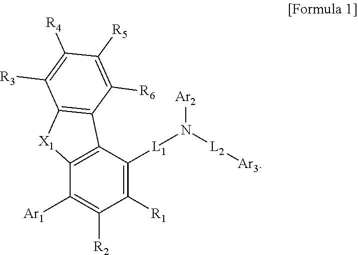

In Formula 1, X₁ may be O or S; L₁ and L₂ may each independently be a direct linkage, a substituted or unsubstituted arylene group having 6 to 15 ring-forming carbon atoms, or a substituted or unsubstituted heteroarylene group having 2 to 15 ring-forming carbon atoms; Ari may be a substituted or unsubstituted aryl group having 6 to 15 ring-forming carbon atoms or a substituted or unsubstituted heteroaryl group having 2 to 15 ring-forming carbon atoms; Ar₂ may be a group represented by Formula 2; Ar₃ may be a substituted or unsubstituted aryl group having 6 to 30 ring-forming carbon atoms or a substituted or unsubstituted heteroaryl group having 2 to 30 ring-forming carbon atoms; and R₁ to R₆ may each independently be a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms.

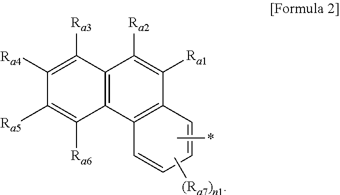

In Formula 2, n1 may be an integer from 0 to 3; Rₐ₁ to Rₐ₇ may each independently be a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms; and [symbol] represents a bond to a neighboring atom.

In an embodiment, the amine compound may be represented by one of Formulas 1-B1 to 1-B4:

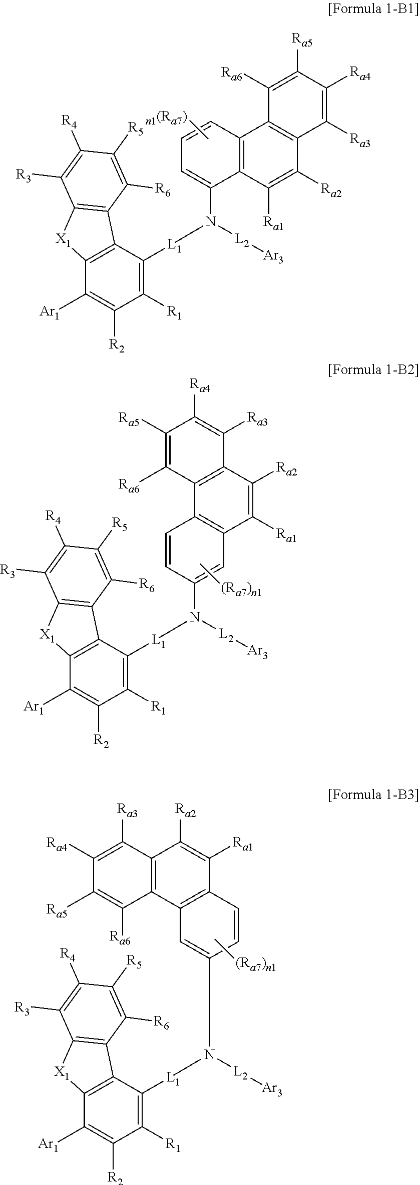

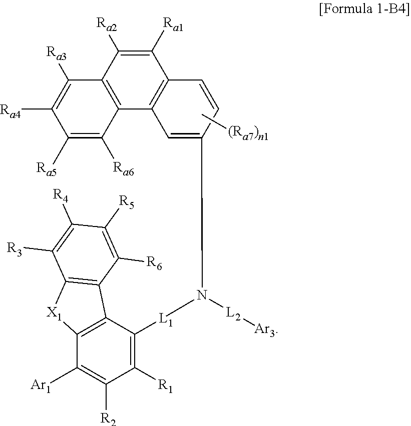

In Formula 1-B1 to Formula 1-B4, L₁, L₂, Aᵣ₁, Ar₂, Ar₃, R₁ to R₆, Rₐ₁ to Rₐ₇, and n1 are the same as defined in Formulas 1 and 2.

In an embodiment, in Formulas 1-B1 to 1-B4, Rₐ₁ to Rₐ₇ may each independently be a hydrogen atom or a deuterium atom.

In an embodiment, in Formula 1, Ar₁ may be a group represented by one of Formulas AR-1 to AR-4:

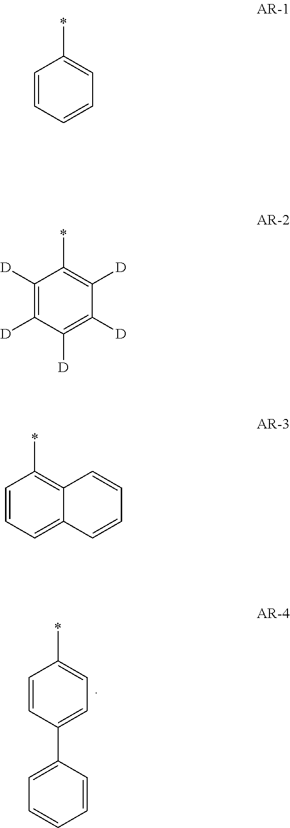 [SMILES: *c1ccccc1 ; *c1c([2H])c([2H])c([2H])c([2H])c1[2H] ; *c1cccc2ccccc12 ; *c1ccc(-c2ccccc2)cc1]

In Formula AR-2, D is a deuterium atom.

In an embodiment, in Formula 1, L₁ may be a direct linkage, a substituted or unsubstituted phenylene group, a substituted or unsubstituted naphthylene group, a substituted or unsubstituted divalent dibenzofuran group, or a substituted or unsubstituted divalent dibenzothiophene group.

In an embodiment, in Formula 1, L₁ may be direct linkage or a group represented by one of Formulas L1-1 to L1-5.

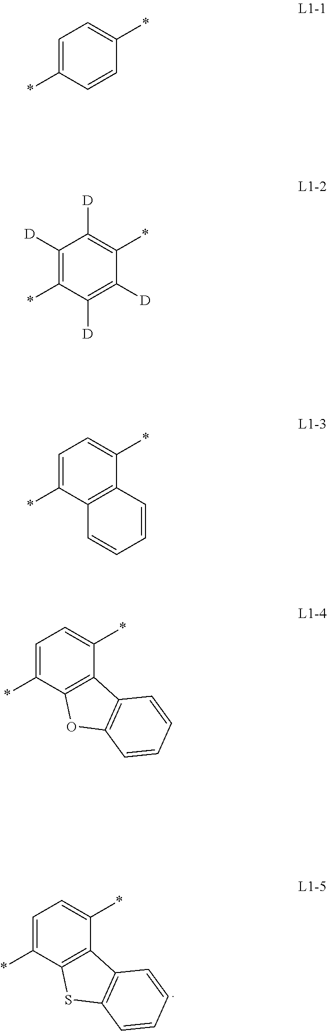

In Formula L1-2, D is a deuterium atom.

In an embodiment, in Formula 1, L₂ may be a direct linkage, a substituted or unsubstituted phenylene group, a substituted or unsubstituted biphenylene group, a substituted or unsubstituted naphthylene group, a substituted or unsubstituted divalent carbazole group, a substituted or unsubstituted divalent dibenzofuran group, or a substituted or unsubstituted divalent dibenzothiophene group.

In an embodiment, in Formula 1, Ar₃ may be a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted carbazole group, a substituted or unsubstituted dibenzofuran group, or substituted or unsubstituted dibenzothiophene group.

In an embodiment, in Formula 1, *-L₂-Ar₃ may be a moiety represented by one Formulas LA-1 to LA-79:

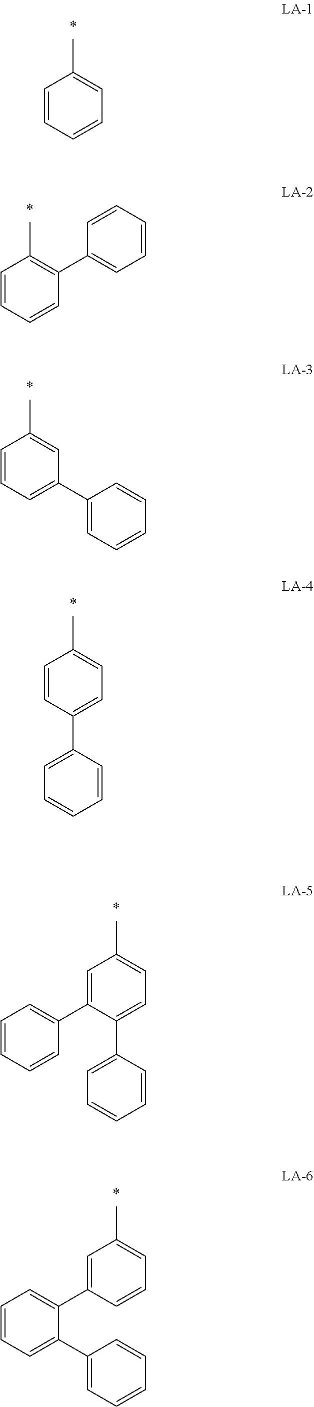

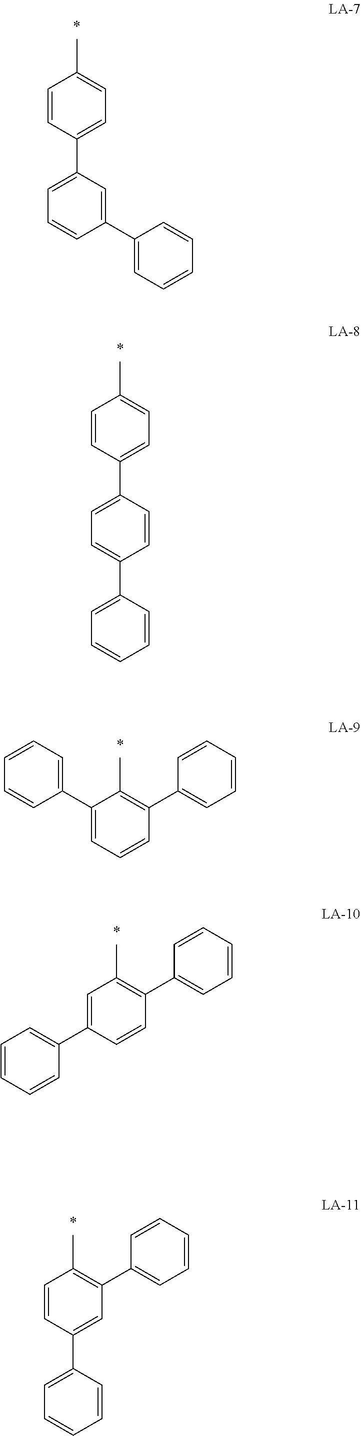

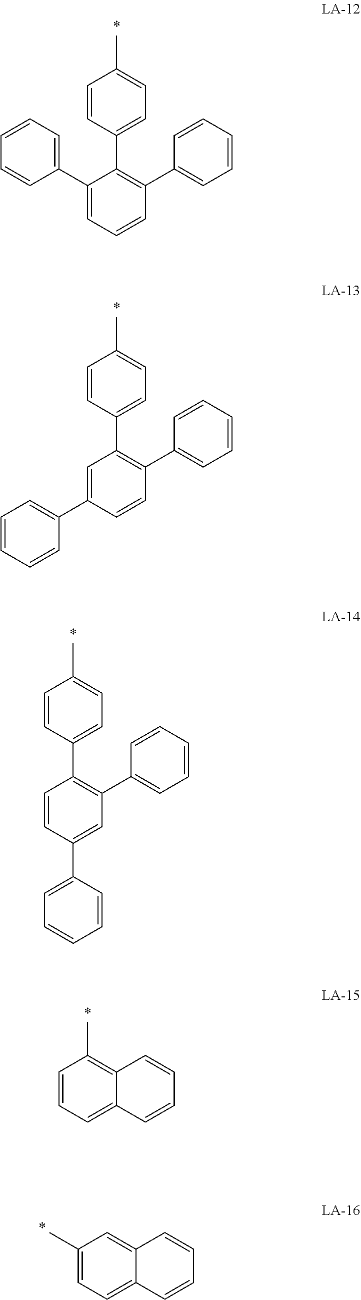

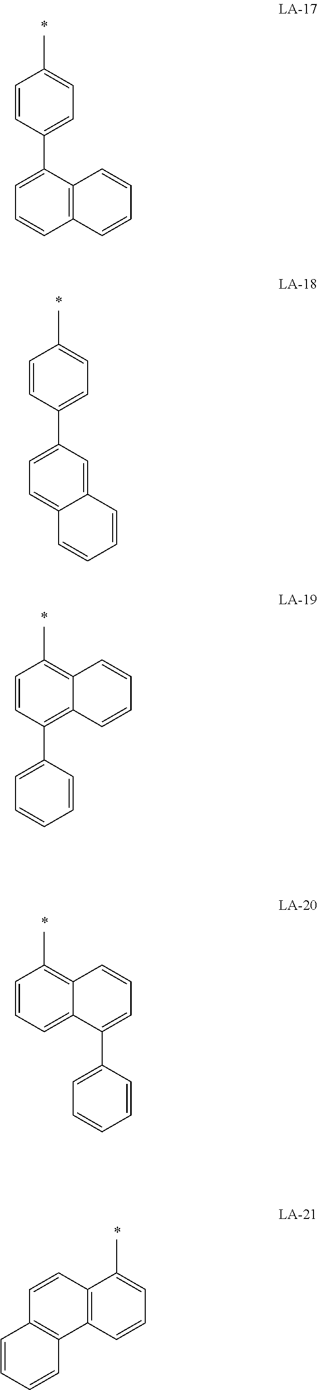

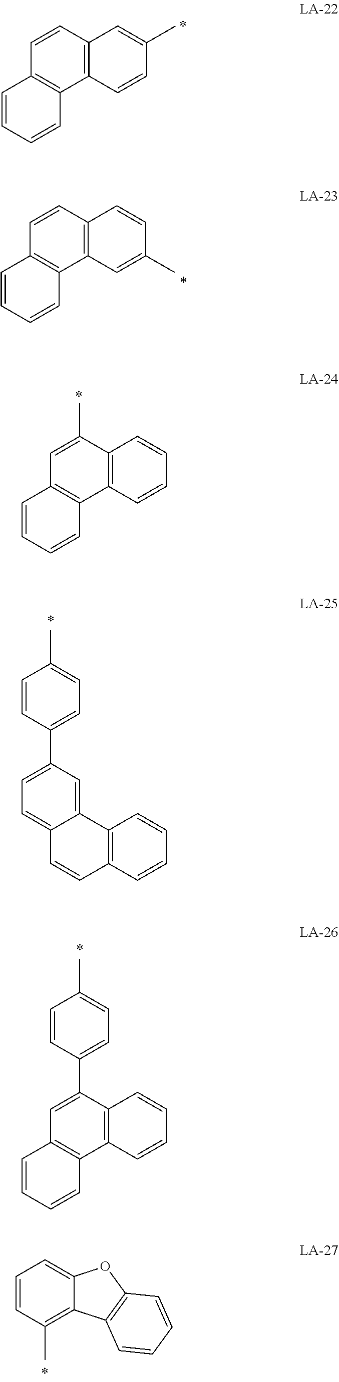

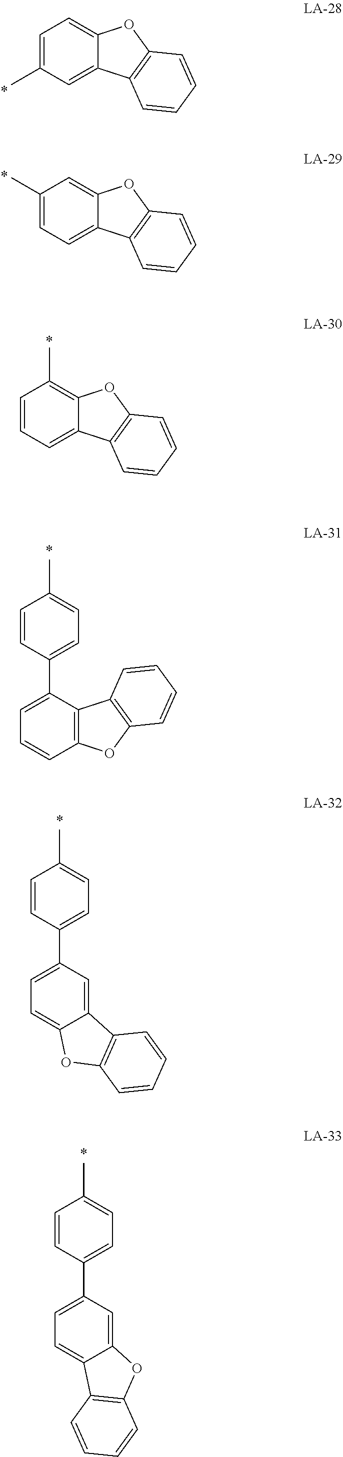 [SMILES: Cc1ccc2oc3ccccc3c2c1 ; Cc1ccc2c(c1)oc1ccccc12 ; Cc1cccc2c1oc1ccccc12 ; Cc1ccc(-c2cccc3oc4ccccc4c23)cc1 ; Cc1ccc(-c2ccc3oc4ccccc4c3c2)cc1 ; Cc1ccc(-c2ccc3c(c2)oc2ccccc23)cc1]

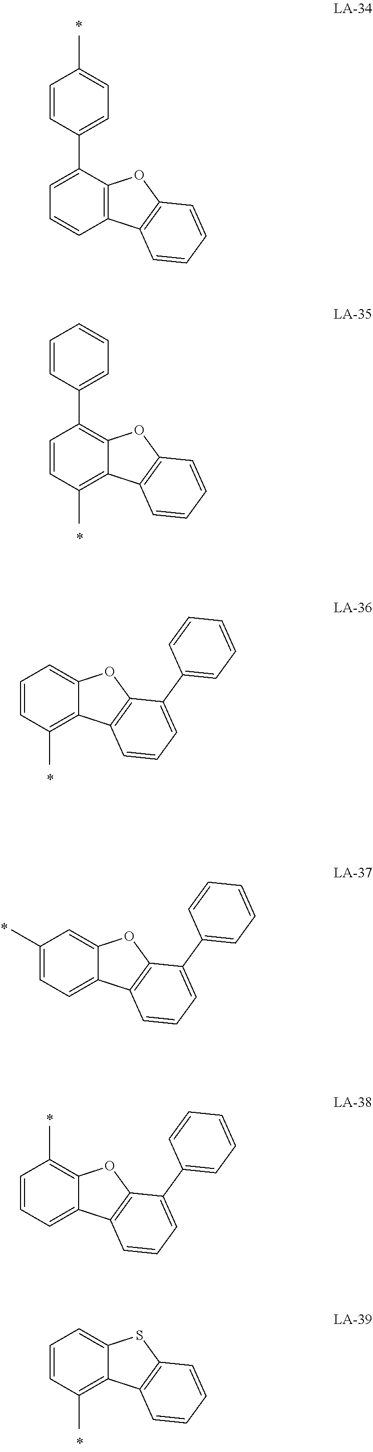 [SMILES: Cc1ccc(-c2cccc3c2oc2ccccc23)cc1 ; Cc1ccc(-c2ccccc2)c2oc3ccccc3c12 ; Cc1cccc2oc3c(-c4ccccc4)cccc3c12 ; Cc1ccc2c(c1)oc1c(-c3ccccc3)cccc12 ; Cc1cccc2c1oc1c(-c3ccccc3)cccc12 ; Cc1cccc2sc3ccccc3c12]

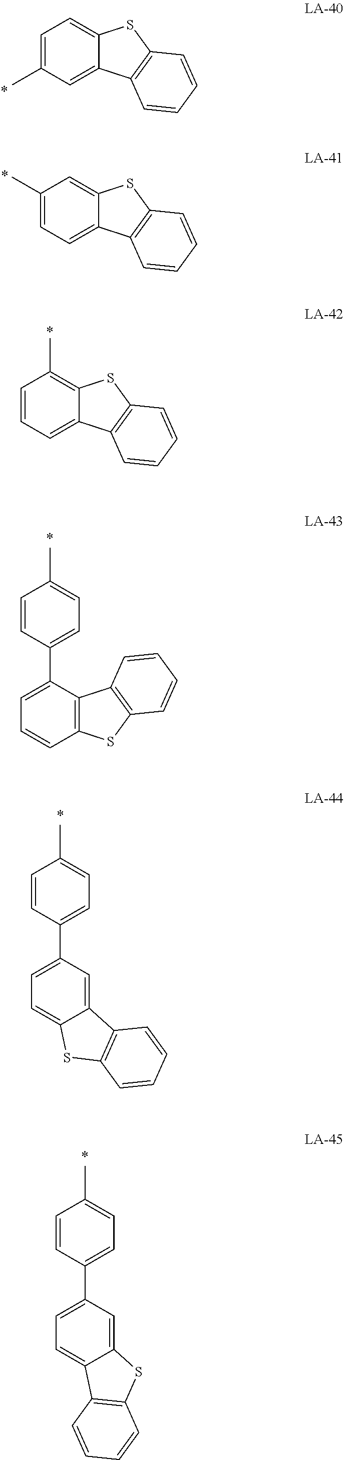 [SMILES: Cc1ccc2sc3ccccc3c2c1 ; Cc1ccc2c(c1)sc1ccccc12 ; Cc1cccc2c1sc1ccccc12 ; Cc1ccc(-c2cccc3sc4ccccc4c23)cc1 ; Cc1ccc(-c2ccc3sc4ccccc4c3c2)cc1 ; Cc1ccc(-c2ccc3c(c2)sc2ccccc23)cc1]

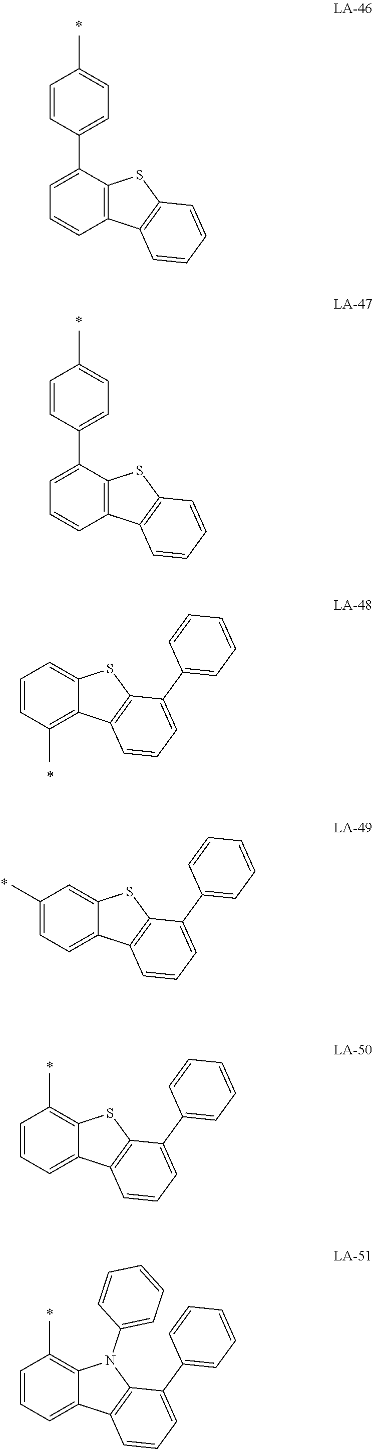 [SMILES: Cc1ccc(-c2cccc3c2sc2ccccc23)cc1 ; Cc1ccc(-c2cccc3c2sc2ccccc23)cc1 ; Cc1cccc2sc3c(-c4ccccc4)cccc3c12 ; Cc1ccc2c(c1)sc1c(-c3ccccc3)cccc12 ; Cc1cccc2c1sc1c(-c3ccccc3)cccc12 ; Cc1cccc2c3cccc(-c4ccccc4)c3n(-c3ccccc3)c12]

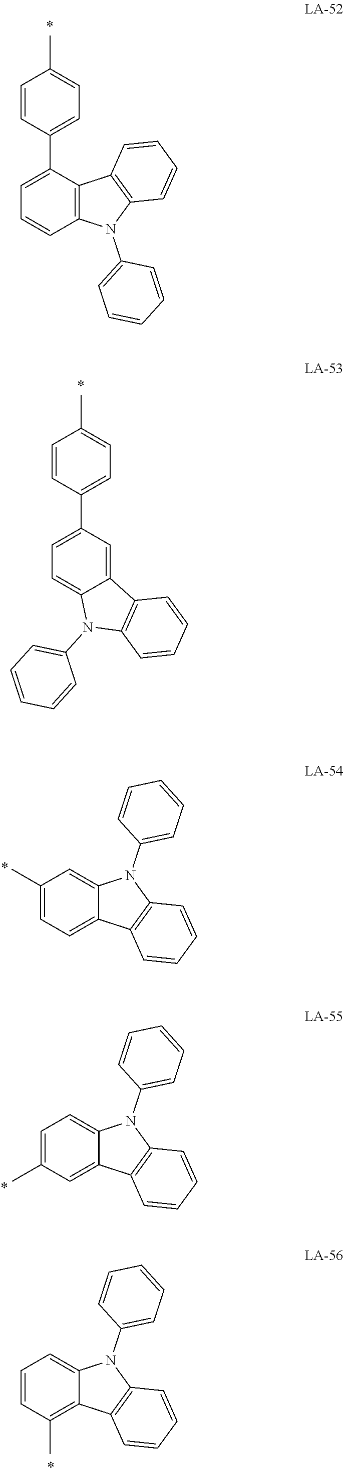 [SMILES: Cc1ccc(-c2cccc3c2c2ccccc2n3-c2ccccc2)cc1 ; Cc1ccc(-c2ccc3c(c2)c2ccccc2n3-c2ccccc2)cc1 ; Cc1ccc2c3ccccc3n(-c3ccccc3)c2c1 ; Cc1ccc2c(c1)c1ccccc1n2-c1ccccc1 ; Cc1cccc2c1c1ccccc1n2-c1ccccc1]

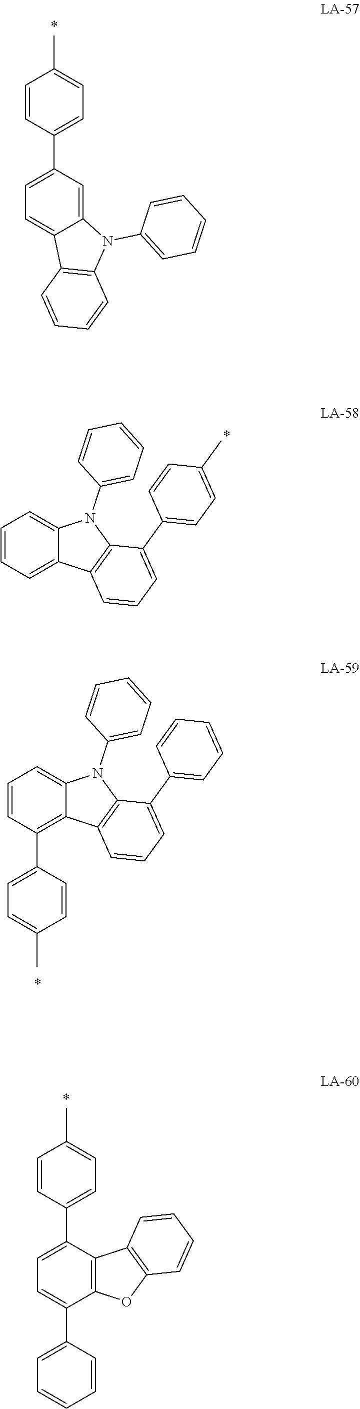 [SMILES: Cc1ccc(-c2ccc3c4ccccc4n(-c4ccccc4)c3c2)cc1 ; Cc1ccc(-c2cccc3c4ccccc4n(-c4ccccc4)c23)cc1 ; Cc1ccc(-c2cccc3c2c2cccc(-c4ccccc4)c2n3-c2ccccc2)cc1 ; Cc1ccc(-c2ccc(-c3ccccc3)c3oc4ccccc4c23)cc1]

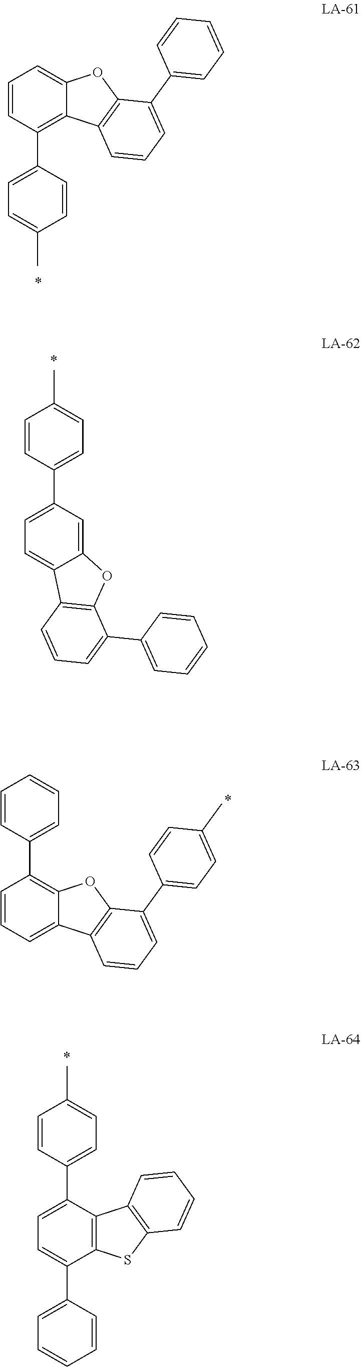 [SMILES: Cc1ccc(-c2cccc3oc4c(-c5ccccc5)cccc4c23)cc1 ; Cc1ccc(-c2ccc3c(c2)oc2c(-c4ccccc4)cccc23)cc1 ; Cc1ccc(-c2cccc3c2oc2c(-c4ccccc4)cccc23)cc1 ; Cc1ccc(-c2ccc(-c3ccccc3)c3sc4ccccc4c23)cc1]

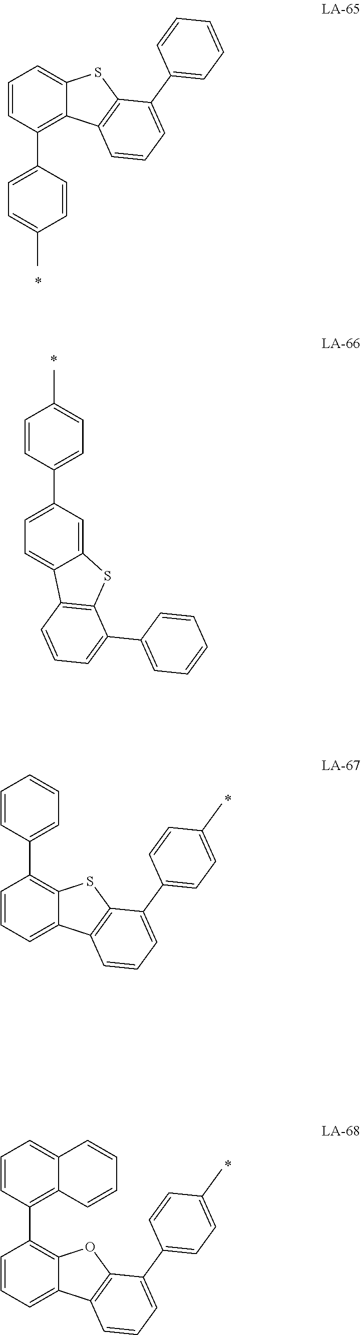 [SMILES: Cc1ccc(-c2cccc3sc4c(-c5ccccc5)cccc4c23)cc1 ; Cc1ccc(-c2ccc3c(c2)sc2c(-c4ccccc4)cccc23)cc1 ; Cc1ccc(-c2cccc3c2sc2c(-c4ccccc4)cccc23)cc1 ; Cc1ccc(-c2cccc3c2oc2c(-c4cccc5ccccc45)cccc23)cc1]

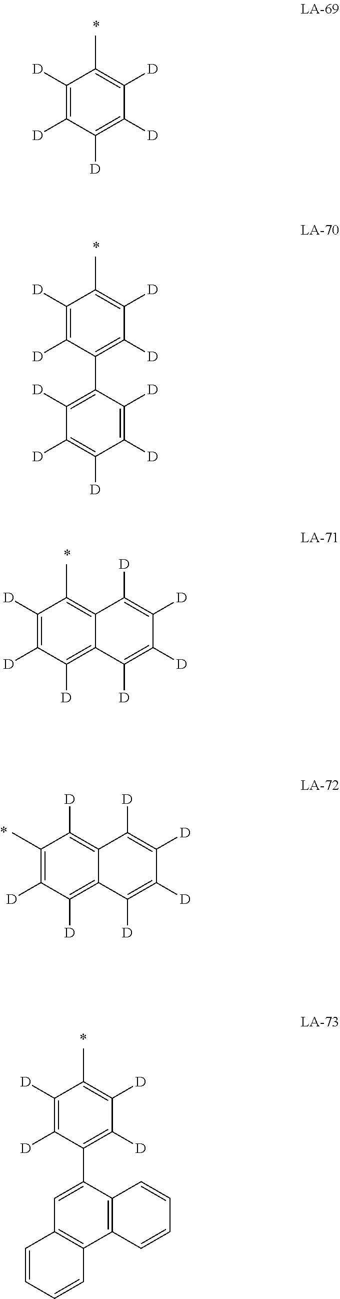 [SMILES: [2H]c1c([2H])c([2H])c(C)c([2H])c1[2H] ; [2H]c1c([2H])c([2H])c(-c2c([2H])c([2H])c(C)c([2H])c2[2H])c([2H])c1[2H] ; [2H]c1c([2H])c([2H])c2c(C)c([2H])c([2H])c([2H])c2c1[2H] ; [2H]c1c([2H])c([2H])c2c([2H])c(C)c([2H])c([2H])c2c1[2H] ; [2H]c1c([2H])c(-c2cc3ccccc3c3ccccc23)c([2H])c([2H])c1C]

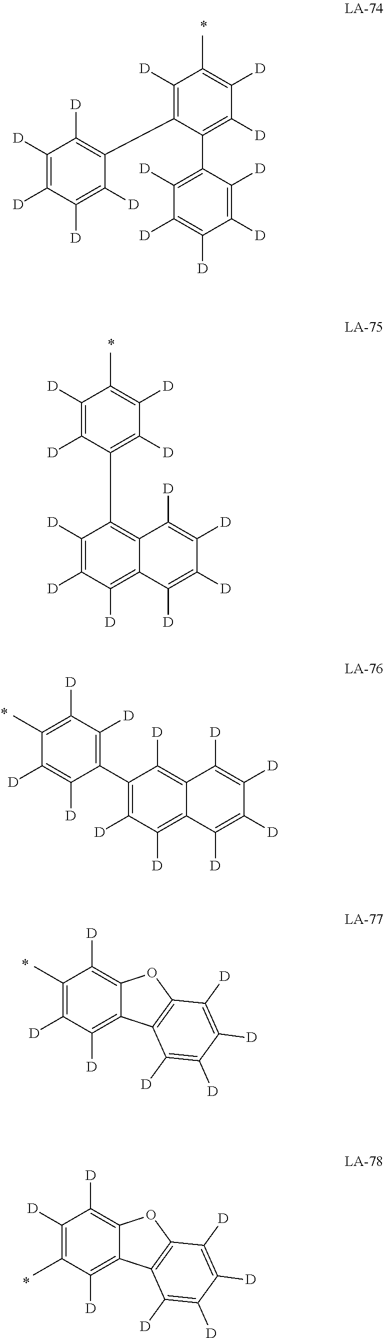 [SMILES: [2H]c1c([2H])c([2H])c(-c2c([2H])c([2H])c(C)c([2H])c2-c2c([2H])c([2H])c([2H])c([2H])c2[2H])c([2H])c1[2H] ; [2H]c1c([2H])c(-c2c([2H])c([2H])c([2H])c3c([2H])c([2H])c([2H])c([2H])c23)c([2H])c([2H])c1C ; [2H]c1c([2H])c(-c2c([2H])c([2H])c3c([2H])c([2H])c([2H])c([2H])c3c2[2H])c([2H])c([2H])c1C ; [2H]c1c([2H])c([2H])c2c(oc3c([2H])c(C)c([2H])c([2H])c32)c1[2H] ; [2H]c1c([2H])c([2H])c2c(oc3c([2H])c([2H])c(C)c([2H])c32)c1[2H]]

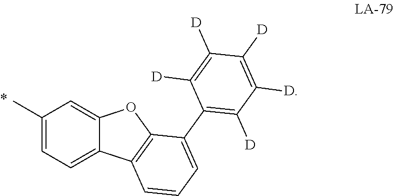

In Formulas LA-69 to LA-79, D is a deuterium atom.

In an embodiment, the amine compound may include at least one compound selected from Compound Group 1, which is explained below.

According to an embodiment, an amine compound may be represented by Formula 1, which is explained herein.

In an embodiment, the amine compound represented by Formula 1 may be represented by one of Formulas 1-B 1 to 1-B4, which are explained herein.

In an embodiment, in Formulas 1-B1 to 1-B4, Rai to Rₐy may each independently be a hydrogen atom or a deuterium atom.

In an embodiment, in Formula 1, An may be a group represented by one of Formulas AR-1 to AR-4, which are explained herein.

In an embodiment, in Formula 1, L₁ may be a direct linkage, a substituted or unsubstituted phenylene group, a substituted or unsubstituted naphthylene group, a substituted or unsubstituted divalent dibenzofuran group, or a substituted or unsubstituted divalent dibenzothiophene group.

In an embodiment, in Formula 1, L₁ may be a direct linkage or a group represented by one of Formulas L1-1 to L1-5, which are explained herein.

In an embodiment, in Formula 1, L₂ may be a direct linkage, a substituted or unsubstituted phenylene group, a substituted or unsubstituted biphenylene group, a substituted or unsubstituted naphthylene group, a substituted or unsubstituted divalent carbazole group, a substituted or unsubstituted divalent dibenzofuran group, or a substituted or unsubstituted divalent dibenzothiophene group; and Ar₃ may be a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted carbazole group, a substituted or unsubstituted dibenzofuran group, or a substituted or unsubstituted dibenzothiophene group.

In an embodiment, in Formula 1, *-L₂-Ar₃ may be a moiety represented by one of Formulas LA-1 to LA-79, which are explained herein.

In an embodiment, the amine compound represented by Formula 1 may be selected from Compound Group 1, which is explained below.

According to an embodiment, a display device may include: a circuit layer disposed on a base layer; and a display element layer disposed on the circuit layer and including a light-emitting element, wherein
the light-emitting element may include a first electrode, a hole transport region disposed on the first electrode, an emission layer disposed on the hole transport region, an electron transport region disposed on the emission layer, and a second electrode disposed on the electron transport region; and the hole transport region may include an amine compound represented by Formula 1, which is explained herein.

It is to be understood that the embodiments above are described in a generic and explanatory sense only and not for the purposes of limitation, and the disclosure is not limited to the embodiments described above.

### BRIEF DESCRIPTION OF THE DRAWINGS

The accompanying drawings are included to provide a further understanding of the embodiments, and are incorporated in and constitute a part of this specification. The drawings illustrate embodiments of the disclosure and principles thereof. The above and other aspects and features of the disclosure will become more apparent by describing in detail embodiments thereof with reference to the accompanying drawings, in which:

FIG. 1 is a schematic plan view of a display device according to an embodiment;

FIG. 2 is a schematic cross-sectional view of a display device according to an embodiment;

FIG. 3 is a schematic cross-sectional view of a light-emitting element according to an embodiment;

FIG. 4 is a schematic cross-sectional view of a light-emitting element according to an embodiment;

FIG. 5 is a schematic cross-sectional view of a light-emitting element according to an embodiment;

FIG. 6 is a schematic cross-sectional view of a light-emitting element according to an embodiment;

FIG. 7 is a schematic cross-sectional view of a display device according to an embodiment;

FIG. 8 is a schematic cross-sectional view of a display device according to an embodiment;

FIG. 9 is a schematic cross-sectional view of a display device according to an embodiment;

FIG. 10 is a schematic cross-sectional view of a display device according to an embodiment; and

FIG. 11 is a schematic diagram of an interior of a vehicle that includes a display device according to an embodiment.

### DETAILED DESCRIPTION OF THE EMBODIMENTS

The disclosure will now be described more fully hereinafter with reference to the accompanying drawings, in which embodiments are shown. This disclosure may, however, be embodied in different forms and should not be construed as limited to the embodiments set forth herein. Rather, these embodiments are provided so that this disclosure will be thorough and complete, and will fully convey the scope of the disclosure to those skilled in the art.

In the drawings, the sizes, thicknesses, ratios, and dimensions of the elements may be exaggerated for ease of description and for clarity. Like reference numbers and reference characters refer to like elements throughout.

In the specification, it will be understood that when an element (or region, layer, part, etc.) is referred to as being “on”, “connected to”, or “coupled to” another element, it can be directly on, connected to, or coupled to the other element, or one or more intervening elements may be present therebetween. In a similar sense, when an element (or region, layer, part, etc.) is described as “covering” another element, it can directly cover the other element, or one or more intervening elements may be present therebetween.

In the specification, when an element is “directly on,” “directly connected to,” or “directly coupled to” another element, there are no intervening elements present. For example, “directly on” may mean that two layers or two elements are disposed without an additional element such as an adhesion element therebetween.

As used herein, the expressions used in the singular such as “a,” “an,” and “the,” are intended to include the plural forms as well, unless the context clearly indicates otherwise.

As used herein, the term “and/or” includes any and all combinations of one or more of the associated listed items. For example, “A and/or B” may be understood to mean “A, B, or A and B.” The terms “and” and “or” may be used in the conjunctive or disjunctive sense and may be understood to be equivalent to “and/of”.

In the specification and the claims, the term “at least one of” is intended to include the meaning of “at least one selected from the group consisting of” for the purpose of its meaning and interpretation. For example, “at least one of A, B, and C” may be understood to mean A only, B only, C only, or any combination of two or more of A, B, and C, such as ABC, ACC, BC, or CC. When preceding a list of elements, the term, “at least one of,” modifies the entire list of elements and does not modify the individual elements of the list.

It will be understood that, although the terms first, second, etc. may be used herein to describe various elements, these elements should not be limited by these terms. These terms are only used to distinguish one element from another element. Thus, a first element could be termed a second element without departing from the teachings of the disclosure. Similarly, a second element could be termed a first element, without departing from the scope of the disclosure.

The spatially relative terms “below”, “beneath”, “lower”, “above”, “upper”, or the like, may be used herein for ease of description to describe the relations between one element or component and another element or component as illustrated in the drawings. It will be understood that the spatially relative terms are intended to encompass different orientations of the device in use or operation, in addition to the orientation depicted in the drawings. For example, in the case where a device illustrated in the drawing is turned over, the device positioned “below” or “beneath” another device may be placed “above” another device.

Accordingly, the illustrative term “below” may include both the lower and upper positions. The device may also be oriented in other directions and thus the spatially relative terms may be interpreted differently depending on the orientations.

The terms “about” or “approximately” as used herein is inclusive of the stated value and means within an acceptable range of deviation for the recited value as determined by one of ordinary skill in the art, considering the measurement in question and the error associated with measurement of the recited quantity (i.e., the limitations of the measurement system). For example, “about” may mean within one or more standard deviations, or within ±20%, ±10%, or ±5% of the stated value.

It should be understood that the terms “comprises,” “comprising,” “includes,” “including,” “have,” “having,” “contains,” “containing,” and the like are intended to specify the presence of stated features, integers, steps, operations, elements, components, or combinations thereof in the disclosure, but do not preclude the presence or addition of one or more other features, integers, steps, operations, elements, components, or combinations thereof.

Unless otherwise defined or implied herein, all terms (including technical and scientific terms) used have the same meaning as commonly understood by those skilled in the art to which this disclosure pertains. It will be further understood that terms, such as those defined in commonly used dictionaries, should be interpreted as having a meaning that is consistent with their meaning in the context of the relevant art and should not be interpreted in an ideal or excessively formal sense unless clearly defined in the specification.

In the specification, the term “substituted or unsubstituted” may describe a group that is substituted or unsubstituted with at least one substituent selected from the group consisting of a deuterium atom, a halogen atom, a cyano group, a nitro group, an amine group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. Each of the substituents listed above may itself be substituted or unsubstituted. For example, a biphenyl group may be interpreted as an aryl group, or it may be interpreted as a phenyl group substituted with a phenyl group.

In the specification, the phrase “bonded to an adjacent group to form a ring” may refer to a group that is bonded to an adjacent group to form a substituted or unsubstituted hydrocarbon ring or a substituted or unsubstituted heterocycle. The hydrocarbon ring may be aliphatic or aromatic. The heterocycle may be aliphatic or aromatic. The hydrocarbon ring and the heterocycle may each independently be monocyclic or polycyclic. A ring that is formed by adjacent groups being bonded to each other may itself be connected to another ring to form a spiro structure.

In the specification, the term “adjacent group” may be interpreted as a substituent that is substituted for an atom which is directly linked to an atom substituted with a corresponding substituent, as another substituent that is substituted for an atom which is substituted with a corresponding substituent, or as a substituent that is sterically positioned at the nearest position to a corresponding substituent. For example, two methyl groups in 1,2-dimethylbenzene may be interpreted as “adjacent groups” to each other, and two ethyl groups in 1,1-diethylcyclopentane may be interpreted as “adjacent groups” to each other. For example, two methyl groups in 4,5-dimethylphenanthrene may be interpreted as “adjacent groups” to each other.

In the specification, examples of a halogen atom may include a fluorine atom, a chlorine atom, a bromine atom, and an iodine atom.

In the specification, an alkyl group may be linear or branched. The number of carbon atoms in an alkyl group may be 1 to 50, 1 to 30, 1 to 20, 1 to 10, or 1 to 6. Examples of an alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldocecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, etc., but embodiments are not limited thereto.

In the specification, a cycloalkyl group may be a cyclic alkyl group. The number of carbon atoms in a cycloalkyl group may be 3 to 50, 3 to 30, 3 to 20, or 3 to 10. Examples of a cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., but embodiments are not limited thereto.

In the specification, an alkenyl group may be a hydrocarbon group that includes at least one carbon-carbon double bond in the middle or at a terminus of an alkyl group having 2 or more carbon atoms. An alkenyl group may be linear or branched. The number of carbon atoms in an alkenyl group is not particularly limited, and may be 2 to 30, 2 to 20, or 2 to 10. Examples of an alkenyl group may include a vinyl group, a 1-butenyl group, a 1-pentenyl group, a 1,3-butadienyl aryl group, a styrenyl group, a styryl vinyl group, etc., but embodiments are not limited thereto.

In the specification, an alkynyl group may be a hydrocarbon group that includes at least one carbon-carbon triple bond in the middle or at a terminus of an alkyl group having 2 or more carbon atoms. An alkynyl group may be linear or branched. The number of carbon atoms in an alkynyl group is not particularly limited, and may be 2 to 30, 2 to 20, or 2 to 10. Examples of an alkynyl group may include an ethynyl group, a propynyl group, etc., but embodiments are not limited thereto.

In the specification, a hydrocarbon ring group may be any functional group or substituent derived from an aliphatic hydrocarbon ring. For example, a hydrocarbon ring group may be a saturated hydrocarbon ring group having 5 to 20 ring-forming carbon atoms.

In the specification, an aryl group may be any functional group or substituent derived from an aromatic hydrocarbon ring. An aryl group may be monocyclic or polycyclic. The number of ring-forming carbon atoms in an aryl group may be 6 to 60, 6 to 30, 6 to 20, or 6 to 15. Examples of an aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, etc., but embodiments are not limited thereto.

In the specification, a fluorenyl group may be substituted, and two substituents may be bonded to each other to form a spiro structure. Examples of a substituted fluorenyl group may include the groups shown below. However, embodiments are not limited thereto.

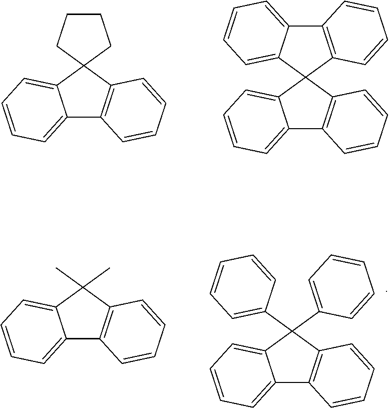

In the specification, a heterocyclic group may be any functional group or substituent derived from a ring that includes at least one of B, O, N, P, Si, and S as a heteroatom. A heterocyclic group may be aliphatic or aromatic. An aromatic heterocyclic group may be a heteroaryl group. An aliphatic heterocycle and an aromatic heterocycle may each independently be monocyclic or polycyclic.

If a heterocyclic group includes two or more heteroatoms, the two or more heteroatoms may be the same as or different from each other. The number of ring-forming carbon atoms in a heterocyclic group may be 2 to 60, 2 to 30, 2 to 20, or 2 to 10.

Examples of an aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., but embodiments are not limited thereto.

Examples of a heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzoimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, etc., but embodiments are not limited thereto.

In the specification, the above description of an aryl group may be applied to an arylene group, except that an arylene group is a divalent group. In the specification, the above description of a heteroaryl group may be applied to a heteroarylene group, except that a heteroarylene group is a divalent group.

In the specification, a silyl group may be an alkylsilyl group or an arylsilyl group. Examples of a silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but embodiments are not limited thereto.

In the specification, the number of carbon atoms in a carbonyl (or acyl) group is not particularly limited, and may be 1 to 40, 1 to 30, or 1 to 20. For example, a carbonyl group may have one of the following structures, but embodiments are not limited thereto.

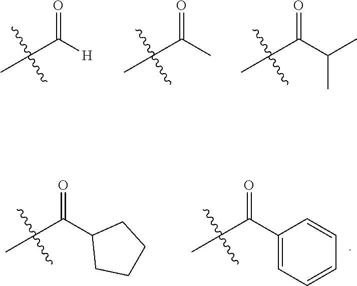

In the specification, the number of carbon atoms in a sulfinyl group or in a sulfonyl group is not particularly limited, and may be 1 to 30. A sulfinyl group may be an alkyl sulfinyl group or an aryl sulfinyl group. A sulfonyl group may be an alkyl sulfonyl group or an aryl sulfonyl group.

In the specification, a thio group may be an alkylthio group or an arylthio group. A thio group may be a sulfur atom that is bonded to an alkyl group or an aryl group as defined above. Examples of a thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but embodiments are not limited thereto.

In the specification, an oxy group may be an oxygen atom that is bonded to an alkyl group or an aryl group as defined above. An oxy group may be an alkoxy group or an aryl oxy group. An alkoxy group may be linear, branched, or cyclic. The number of carbon atoms in an alkoxy group is not particularly limited, and may be, for example, 1 to 20 or 1 to 10. Examples of an oxy group may include a methoxy group, an ethoxy group, an n-propoxy group, an isopropoxy group, a butoxy group, a pentyloxy group, a hexyloxy group, an octyloxy group, a nonyloxy group, a decyloxy group, a benzyloxy group, etc., but embodiments are not limited thereto.

In the specification, a boron group may be a boron atom that is bonded to an alkyl group or an aryl group as defined above. A boron group may be an alkyl boron group or an aryl boron group. Examples of a boron group may include a dimethylboron group, a trimethylboron group, a t-butyldimethylboron group, a diphenylboron group, a phenylboron group, etc., but embodiments are not limited thereto.

In the specification, the number of carbon atoms in an amine group is not particularly limited, and may be 1 to 30. An amine group may be an alkyl amine group or an aryl amine group. Examples of an amine group may include a methylamine group, a dimethylamine group, a phenylamine group, a diphenylamine group, a naphthylamine group, a 9-methyl-anthracenylamine group, etc., but embodiments are not limited thereto.

In the specification, an alkyl group within an alkoxy group, an alkylthio group, an alkylsulfoxy group, an alkylaryl group, an alkylamino group, an alkyl boron group, an alkyl silyl group, or an alkyl amine group may be the same as an examples of an alkyl group as described above.

In the specification, an aryl group within an aryloxy group, an arylthio group, an arylsulfoxy group, an alkylaryl group, an arylamino group, an arylboron group, an arylsilyl group, or an arylamine group may be the same as an example of an aryl group as described above.

In the specification, a direct linkage may be a single bond.

In the specification, the symbols [symbol] and [symbol] each represent a bond to a neighboring atom in a corresponding formula or moiety.

Hereinafter, embodiments of the will be described with reference to the accompanying drawings.

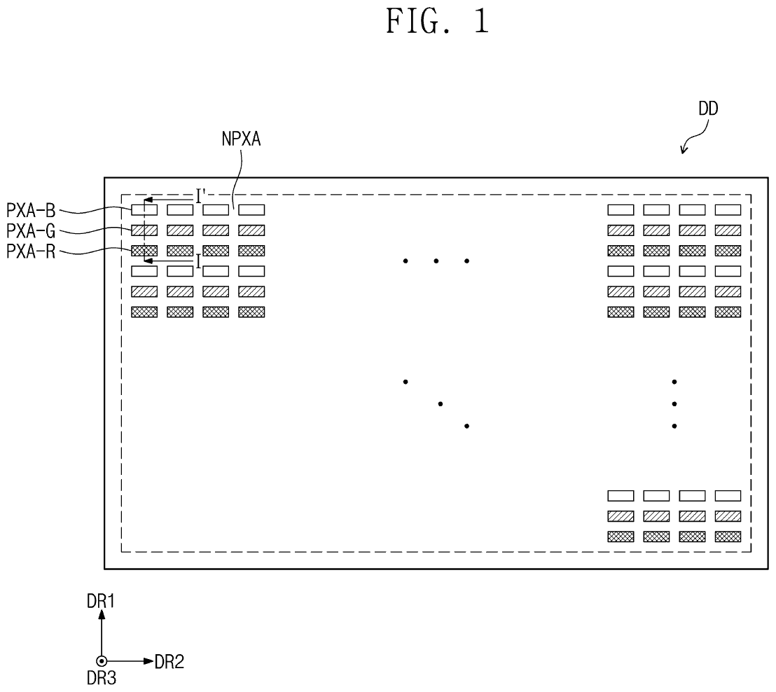

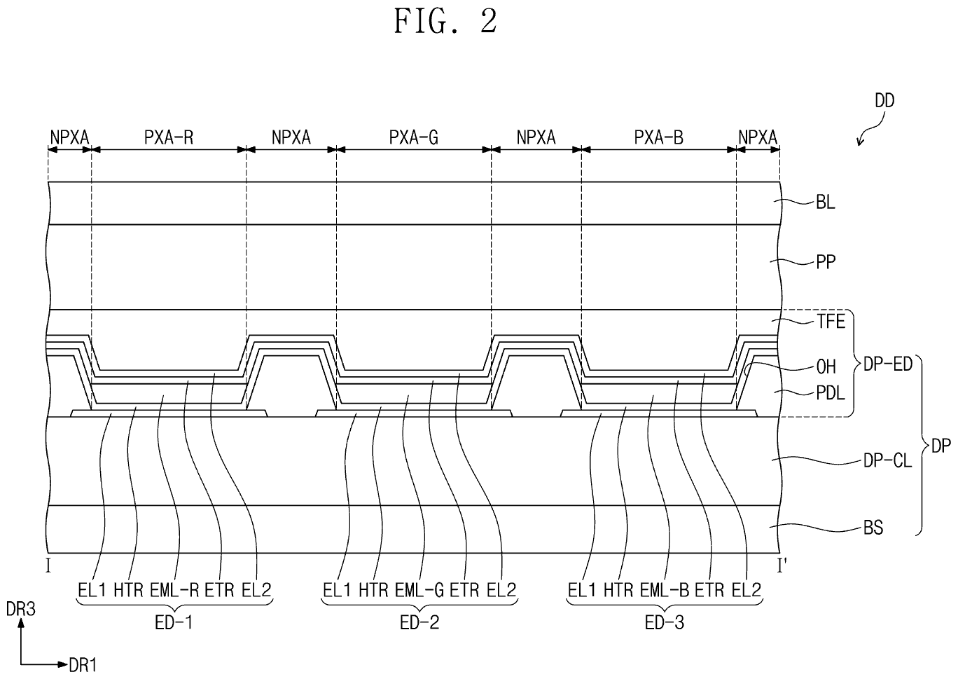

FIG. 1 is a schematic plan view of a display device DD according to an embodiment. FIG. 2 is a schematic cross-sectional view of the display device DD according to an embodiment. FIG. 2 is a schematic cross-sectional view illustrating a section taken along virtual line I-I′ in FIG. 1.

The display device DD may include a display panel DP and an optical layer PP disposed on the display panel DP. The display panel DP includes light-emitting elements ED-1, ED-2, and ED-3. The display device DD may include multiples of each of the light-emitting elements ED-1, ED-2, and ED-3. The optical layer PP may be disposed on the display panel DP to control light that is reflected at the display panel DP from an external light. The optical layer PP may include, for example, a polarization layer or a color filter layer. Although not shown in the drawings, in an embodiment, the optical layer PP may be omitted from the display device DD.

A base substrate BL may be disposed on the optical layer PP. The base substrate BL may provide a base surface on which the optical layer PP is disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

The display device DD according to an embodiment may further include a filling layer (not shown). The filling layer (not shown) may be disposed between a display element layer DP-ED and the base substrate BL. The filling layer (not shown) may be an organic material layer. The filling layer (not shown) may include at least one of an acrylic-based resin, a silicone-based resin, and an epoxy-based resin.

The display panel DP may include a base layer BS, a circuit layer DP-CL provided on the base layer BS, and the display element layer DP-ED. The display element layer DP-ED may include a pixel defining film PDL, light-emitting elements ED-1, ED-2, and ED-3 disposed between portions of the pixel defining film PDL, and an encapsulation layer TFE disposed on the light-emitting elements ED-1, ED-2, and ED-3.

The base layer BS may provide a base surface on which the display element layer DP-ED is disposed. The base layer BS may be a glass substrate, a metal substrate, a plastic substrate, etc. However, embodiments are not limited thereto, and the base layer BS may include an inorganic layer, an organic layer, or a composite material layer.

In an embodiment, the circuit layer DP-CL is disposed on the base layer BS, and the circuit layer DP-CL may include transistors (not shown). The transistors (not shown) may each include a control electrode, an input electrode, and an output electrode. For example, the circuit layer DP-CL may include a switching transistor and a driving transistor for driving the light-emitting elements ED-1, ED-2, and ED-3 of the display element layer DP-ED.

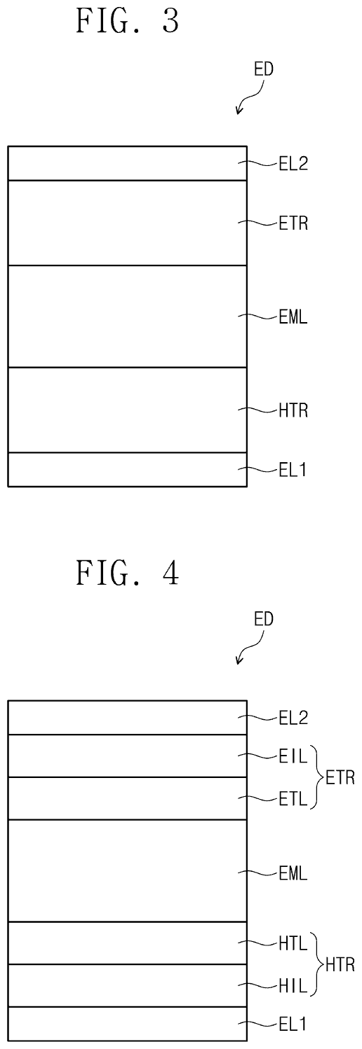

The light-emitting elements ED-1, ED-2, and ED-3 may each have a structure of a light-emitting element ED of an embodiment according to any of FIGS. 3 to 6, which will be described later. The light-emitting elements ED-1, ED-2, and ED-3 may each include a first electrode EL1, a hole transport region HTR, emission layers EML-R, EML-G, and EML-B, an electron transport region ETR, and a second electrode EL2.

FIG. 2 illustrates an embodiment in which the emission layers EML-R, EML-G, and EML-B of the light-emitting elements ED-1, ED-2, and ED-3 are disposed in openings OH defined in the pixel defining film PDL, and the hole transport region HTR, the electron transport region ETR, and the second electrode EL2 are each provided as a common layer for the light-emitting elements ED-1, ED-2, and ED-3. However, embodiments are not limited thereto.

Although not shown in FIG. 2, in an embodiment, the hole transport region HTR and the electron transport region ETR may each be provided by being patterned in the openings OH defined in the pixel defining film PDL. For example, in an embodiment, the hole transport region HTR, the emission layers EML-R, EML-G, and EML-B, and the electron transport region ETR of the light-emitting elements ED-1, ED-2, and ED-3 may each be provided by being patterned through an inkjet printing method.

The encapsulation layer TFE may cover the light-emitting elements ED-1, ED-2, and ED-3. The encapsulation layer TFE may seal the display element layer DP-ED. The encapsulation layer TFE may be a thin film encapsulation layer. The encapsulation layer TFE may be formed of a single layer or of multiple layers. The encapsulation layer TFE may include at least one insulation layer. The encapsulation layer TFE according to an embodiment may include at least one inorganic film (hereinafter, an encapsulation-inorganic film). In an embodiment, the encapsulation layer TFE may include at least one organic film (hereinafter, an encapsulation-organic film) and at least one encapsulation-inorganic film.

The encapsulation-inorganic film protects the display element layer DP-ED from moisture and/or oxygen, and the encapsulation-organic film protects the display element layer DP-ED from foreign substances such as dust particles. The encapsulation-inorganic film may include silicon nitride, silicon oxynitride, silicon oxide, titanium oxide, aluminum oxide, or the like, but embodiments are not limited thereto. The encapsulation-organic film may include an acrylic-based compound, an epoxy-based compound, or the like. The encapsulation-organic film may include a photopolymerizable organic material, but embodiments are not limited thereto.

The encapsulation layer TFE may be disposed on the second electrode EL2 and may be disposed to fill the openings OH.

Referring to FIGS. 1 and 2, the display device DD may include non-light emitting regions NPXA and light emitting regions PXA-R, PXA-G, and PXA-B. The light emitting regions PXA-R, PXA-G, and PXA-B may each be a region that emits light respectively generated by the light-emitting elements ED-1, ED-2, and ED-3. The light emitting regions PXA-R, PXA-G, and PXA-B may be spaced apart from each other in a plan view.

The light emitting regions PXA-R, PXA-G, and PXA-B may be regions that are separated from each other by the pixel defining film PDL. The non-light emitting regions NPXA may be areas between the adjacent light emitting regions PXA-R, PXA-G, and PXA-B, and which may correspond to the pixel defining film PDL. In an embodiment, the light emitting regions PXA-R, PXA-G, and PXA-B may each correspond to a pixel. The pixel defining film PDL may separate the light-emitting elements ED-1, ED-2, and ED-3. The emission layers EML-R, EML-G, and EML-B of the light-emitting elements ED-1, ED-2, and ED-3 may be disposed in openings OH defined in the pixel defining film PDL and separated from each other.

The light emitting regions PXA-R, PXA-G, and PXA-B may be arranged into groups according to the color of light generated from the light-emitting elements ED-1, ED-2, and ED-3. In the display device DD according to an embodiment illustrated in FIGS. 1 and 2, three light emitting regions PXA-R, PXA-G, and PXA-B, which respectively emit red light, green light, and blue light, are illustrated as an example. For example, the display device DD may include a red light emitting region PXA-R, a green light emitting region PXA-G, and a blue light emitting region PXA-B, which are distinct from each other.

In the display device DD according to an embodiment, the light-emitting elements ED-1, ED-2, and ED-3 may emit light having wavelengths that are different from each other. For example, in an embodiment, the display device DD may include a first light-emitting element ED-1 that emits red light, a second light-emitting element ED-2 that emits green light, and a third light-emitting element ED-3 that emits blue light. For example, the red light emitting region PXA-R, the green light emitting region PXA-G, and the blue light emitting region PXA-B of the display device DD may respectively correspond to the first light-emitting element ED-1, the second light-emitting element ED-2, and the third light-emitting element ED-3.

However, embodiments are not limited thereto, and the first to third light-emitting elements ED-1, ED-2, and ED-3 may emit light in a same wavelength range, or at least one light-emitting element may emit light in a wavelength range that is different from the remainder. For example, the first to third light-emitting elements ED-1, ED-2, and ED-3 may each emit blue light.

The light emitting regions PXA-R, PXA-G, and PXA-B in the display device DD according to an embodiment may be arranged in a stripe configuration. Referring to FIG. 1, the red light emitting regions PXA-R, the green light emitting regions PXA-G, and the blue light emitting regions PXA-B may be respectively arranged along a second directional axis DR2. In another embodiment, the red light emitting region PXA-R, the green light emitting region PXA-G, and the blue light emitting region PXA-B may be arranged in this repeating order along a first directional axis DR1.

FIGS. 1 and 2 illustrate that the light emitting regions PXA-R, PXA-G, and PXA-B all have a similar area, but embodiments are not limited thereto. In an embodiment, the light emitting regions PXA-R, PXA-G, and PXA-B may be different in size or shape from each other, according to a wavelength range of emitted light. The areas of the light emitting regions PXA-R, PXA-G, and PXA-B may be areas in a plan view that are defined by the first directional axis DR1 and the second directional axis DR2.

An arrangement of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in various combinations, according to the display quality characteristics that are required for the display device DD. For example, the light emitting regions PXA-R, PXA-G, and PXA-B may be arranged in a pentile configuration (such as PenTile®) or in a diamond configuration (such as Diamond Pixel®).

The areas of the light emitting regions PXA-R, PXA-G, and PXA-B may be different in size from each other. For example, in an embodiment, an area of a green light emitting region PXA-G may be smaller than an area of a blue light emitting region PXA-B, but embodiments are not limited thereto.

Hereinafter, FIG. 3 to FIG. 6 are each a schematic cross-sectional view of a light-emitting element according to an embodiment. In an embodiment shown in FIG. 3, a light-emitting element ED may include a first electrode EL1, a hole transport region HTR, an emission layer EML, an electron transport region ETR, and a second electrode EL2.

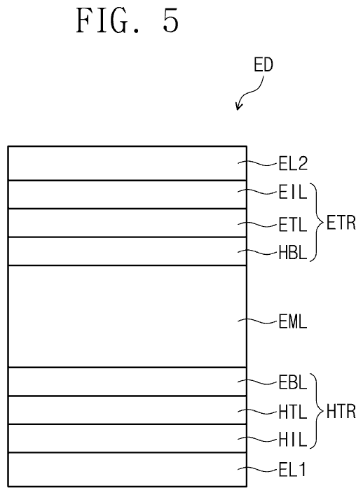

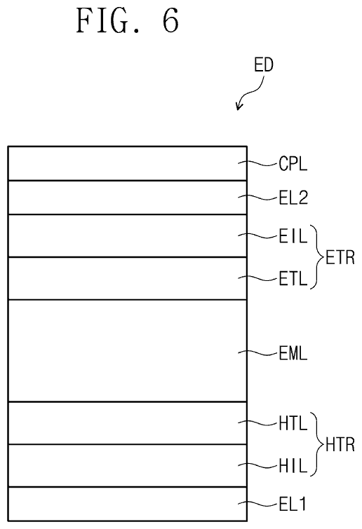

In comparison to FIG. 3, FIG. 4 is a schematic cross-sectional view of a light-emitting element ED according to an embodiment, in which a hole transport region HTR includes a hole injection layer HIL and a hole transport layer HTL, and an electron transport region ETR includes an electron injection layer EIL and an electron transport layer ETL. In comparison to FIG. 3, FIG. 5 is a schematic cross-sectional view of a light-emitting element ED according to an embodiment, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. In comparison to FIG. 4, FIG. 6 is a schematic cross-sectional view of a light-emitting element ED according to an embodiment that further includes a capping layer CPL disposed on a second electrode EL2.

The first electrode EL1 has conductivity. The first electrode EL1 may be formed of a metal material, a metal alloy, or a conductive compound. The first electrode EL1 may be an anode or a cathode. However, embodiments are not limited thereto. In an embodiment, the first electrode EL1 may be a pixel electrode. The first electrode EL1 may be a transmissive electrode, a transflective electrode, or a reflective electrode. The first electrode EL1 may include at least one of Ag, Mg, Cu, Al, Pt, Pd, Au, Ni, Nd, Ir, Cr, Li, Ca, LiF, Mo, Ti, W, In, Sn, Zn, an oxide thereof, a compound thereof, and a mixture thereof.

If the first electrode EL1 is a transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound thereof, or a mixture thereof (e.g., a mixture of Ag and Mg). In another embodiment, the first electrode EL1 may have a multilayer structure including a reflective film or a transflective film formed of the above-described materials, and a transparent conductive film formed of ITO, IZO, ZnO, ITZO, etc. For example, the first electrode EL1 may have a three-layered structure of ITO/Ag/ITO, but embodiments are not limited thereto. In an embodiment, the first electrode EL1 may include the above-described metal materials, combinations of at least two metal materials of the above-described metal materials, oxides of the above-described metal materials, or the like. A thickness of the first electrode EL1 may be in a range of about 700 Å to about 10,000 Å. For example, the thickness of the first electrode EL1 may be in a range of about 1,000 Å to about 3,000 Å.

A light-emitting element ED according to an embodiment may include an amine compound according to an embodiment. In the light-emitting element ED, the hole transport region HTR may include the amine compound according to an embodiment. For example, at least one of a hole injection layer HIL, a hole transport layer HTL, and an electron blocking layer EBL may include the amine compound according to an embodiment.

The amine compound according to an embodiment may be a monoamine compound. In the specification, the term “amine group” does not encompass a carbazole group, and the term “heterocyclic group” encompasses a carbazole group.

The amine compound according to an embodiment may include first, second, and third substituents bonded to an amine group. The first substituent may include a substituted dibenzofuran group or a substituted dibenzothiophene group. The second substituent may include a substituted or unsubstituted phenanthryl group. The third substituent may include a substituted or unsubstituted aryl group having 6 to 30 ring-forming carbon atoms or a substituted or unsubstituted heteroaryl group having 2 to 30 ring-forming carbon atoms. The amine group including the first and second substituents may exhibit excellent material stability and hole transporting properties. Therefore, the light-emitting element ED including the amine compound according to an embodiment may exhibit characteristics of low driving voltage, high efficiency, and long lifespan.

The light-emitting element ED according to an embodiment may include the amine compound according to an embodiment. The amine compound according to an embodiment may be represented by Formula 1:

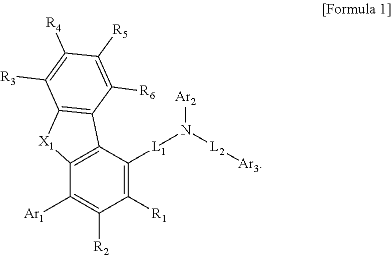

In Formula 1, X₁ may be O or S. A fused tricyclic ring including X₁ may correspond to the first substituent described above. If X₁ is 0, the first substituent may include a substituted dibenzofuran group. If X₁ is S, the first substituent may include a substituted dibenzothiophene group.

In Formula 1, L₁ and L₂ may each independently be a direct linkage, a substituted or unsubstituted arylene group having 6 to 15 ring-forming carbon atoms, or a substituted or unsubstituted heteroarylene group having 2 to 15 ring-forming carbon atoms. In an embodiment, L₂ may be a direct linkage, a substituted or unsubstituted phenylene group, a substituted or unsubstituted biphenylene group, a substituted or unsubstituted naphthylene group, a substituted or unsubstituted divalent carbazole group, a substituted or unsubstituted divalent dibenzofuran group, or a substituted or unsubstituted divalent dibenzothiophene group. In an embodiment, L₁ may be a direct linkage, a substituted or unsubstituted phenylene group, a substituted or unsubstituted naphthylene group, a substituted or unsubstituted divalent dibenzofuran group, or a substituted or unsubstituted divalent dibenzothiophene group.

In an embodiment, L₁ may be a direct linkage or a group represented by one of Formulas L1-1 to L1-5. In Formula L1-2, D is a deuterium atom.

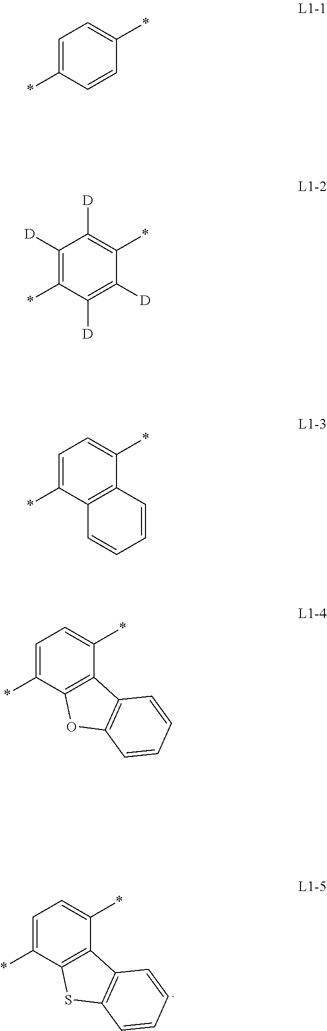 [SMILES: *c1ccc(*)cc1 ; *c1c([2H])c([2H])c(*)c([2H])c1[2H] ; *c1ccc(*)c2ccccc12 ; *c1ccc(*)c2c1oc1ccccc12 ; *c1ccc(*)c2c1sc1ccccc12]

In Formula 1, Ar₁ may be a substituted or unsubstituted aryl group having 6 to 15 ring-forming carbon atoms or a substituted or unsubstituted heteroaryl group having 2 to 15 ring-forming carbon atoms. For example, Ar₁ may be a substituted or unsubstituted phenyl group, a substituted or unsubstituted biphenyl group, or a substituted or unsubstituted naphthyl group.

In an embodiment, Ar₁ may be a group represented by one of Formulas AR-1 to AR-4. In Formula AR-2, D is a deuterium atom.

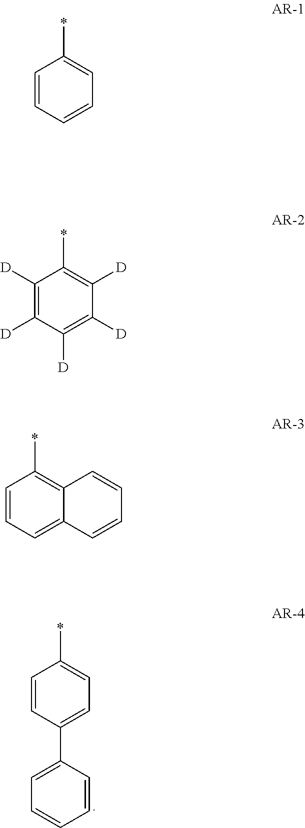 [SMILES: *c1ccccc1 ; *c1c([2H])c([2H])c([2H])c([2H])c1[2H] ; *c1cccc2ccccc12 ; *c1ccc(-c2ccccc2)cc1]

In Formula 1, Ar₃ may be a substituted or unsubstituted aryl group having 6 to 30 ring-forming carbon atoms or a substituted or unsubstituted heteroaryl group having 2 to 30 ring-forming carbon atoms. Ar₃ may correspond to the third substituent described above. In an embodiment, Ar₃ may be a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted carbazole group, a substituted or unsubstituted dibenzofuran group, or a substituted or unsubstituted dibenzothiophene group.

In an embodiment, in Formula 1, *-L₂-Ar₃ may be a moiety represented by one of Formulas LA-1 to LA-79. In Formula LA-69 to LA-79, D is a deuterium atom.

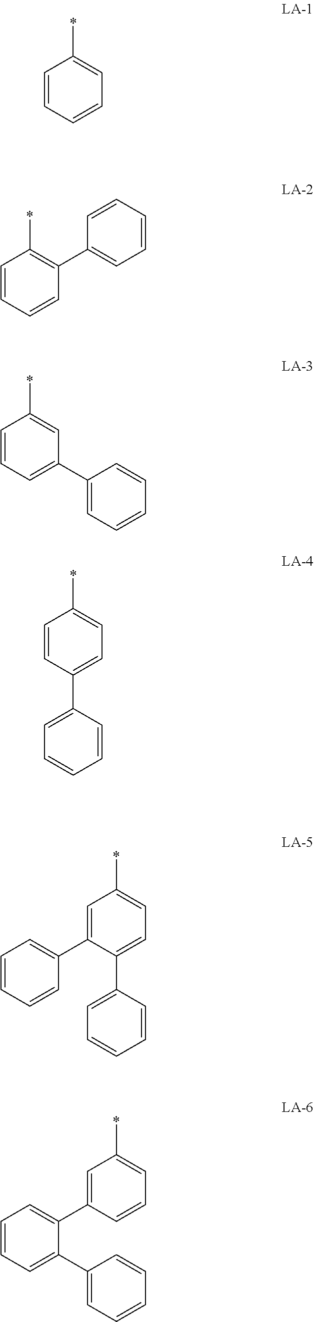 [SMILES: *c1ccccc1 ; *c1ccccc1-c1ccccc1 ; *c1cccc(-c2ccccc2)c1 ; *c1ccc(-c2ccccc2)cc1 ; *c1ccc(-c2ccccc2)c(-c2ccccc2)c1 ; *c1cccc(-c2ccccc2-c2ccccc2)c1]

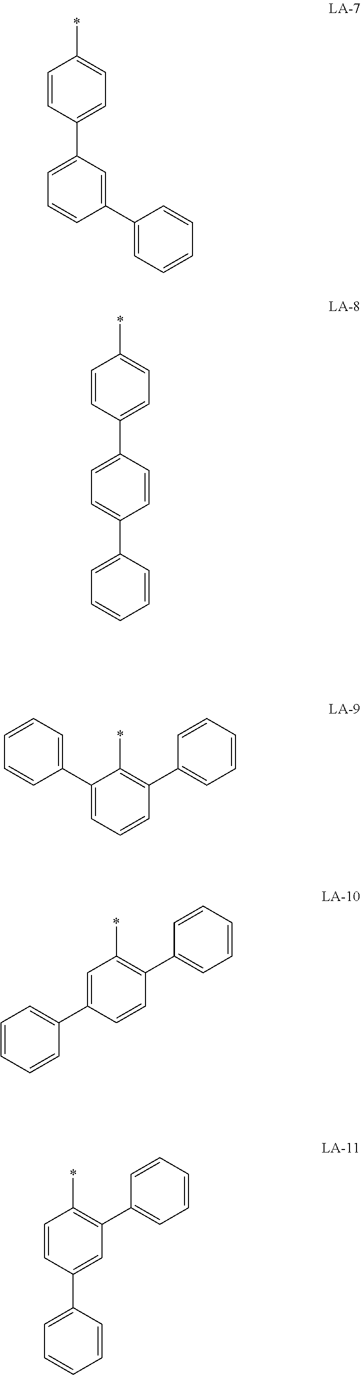 [SMILES: *c1ccc(-c2cccc(-c3ccccc3)c2)cc1 ; *c1ccc(-c2ccc(-c3ccccc3)cc2)cc1 ; *c1c(-c2ccccc2)cccc1-c1ccccc1 ; *c1cc(-c2ccccc2)ccc1-c1ccccc1 ; *c1ccc(-c2ccccc2)cc1-c1ccccc1]

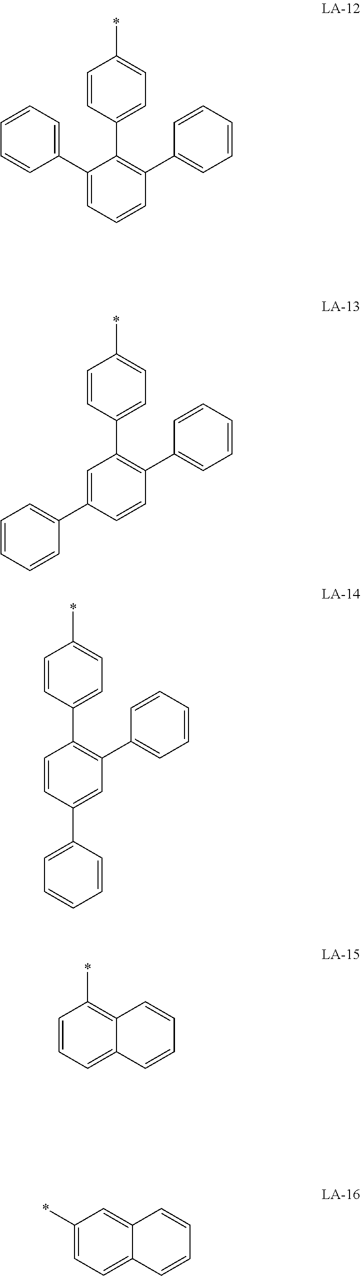 [SMILES: *c1ccc(-c2c(-c3ccccc3)cccc2-c2ccccc2)cc1 ; *c1ccc(-c2cc(-c3ccccc3)ccc2-c2ccccc2)cc1 ; *c1ccc(-c2ccc(-c3ccccc3)cc2-c2ccccc2)cc1 ; *c1cccc2ccccc12 ; *c1ccc2ccccc2c1]

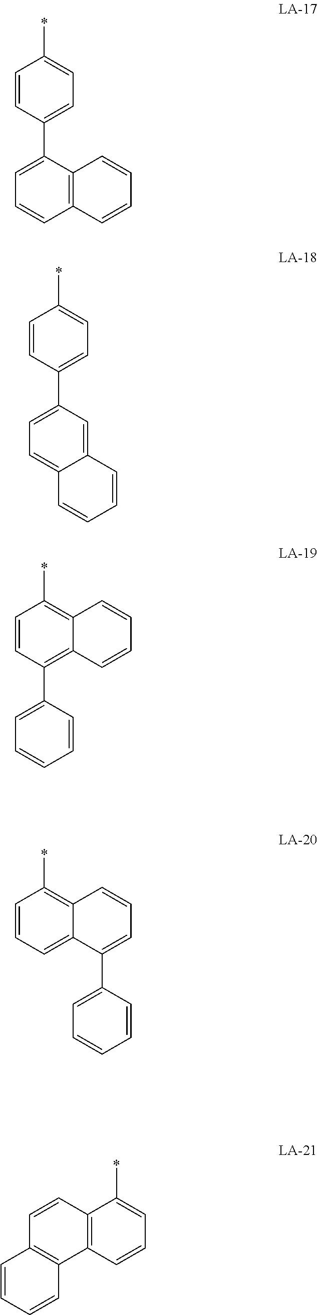 [SMILES: *c1ccc(-c2cccc3ccccc23)cc1 ; *c1ccc(-c2ccc3ccccc3c2)cc1 ; *c1ccc(-c2ccccc2)c2ccccc12 ; *c1cccc2c(-c3ccccc3)cccc12 ; *c1cccc2c1ccc1ccccc12]

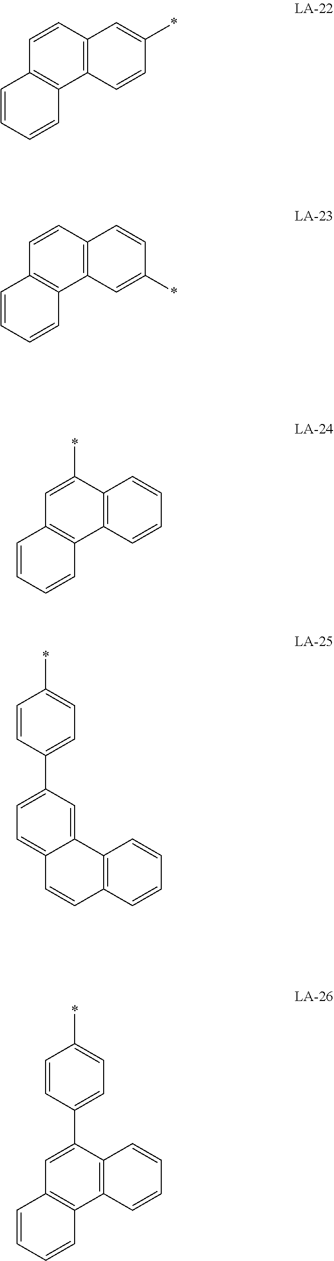 [SMILES: *c1ccc2c(ccc3ccccc32)c1 ; *c1ccc2ccc3ccccc3c2c1 ; *c1cc2ccccc2c2ccccc12 ; *c1ccc(-c2ccc3ccc4ccccc4c3c2)cc1 ; *c1ccc(-c2cc3ccccc3c3ccccc23)cc1]

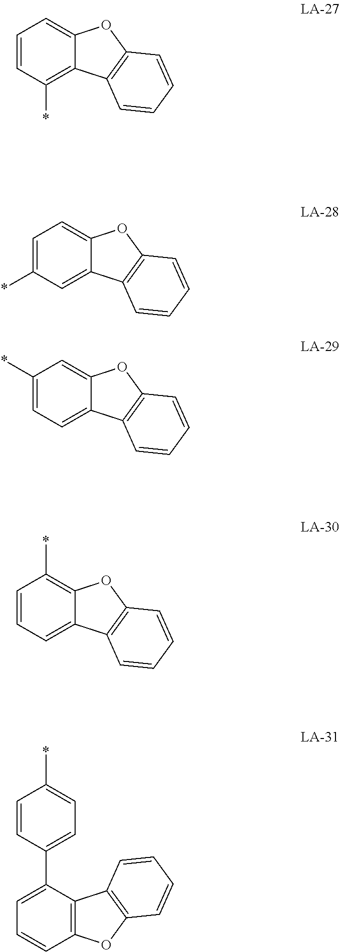 [SMILES: *c1cccc2oc3ccccc3c12 ; *c1ccc2oc3ccccc3c2c1 ; *c1ccc2c(c1)oc1ccccc12 ; *c1cccc2c1oc1ccccc12 ; *c1ccc(-c2cccc3oc4ccccc4c23)cc1]

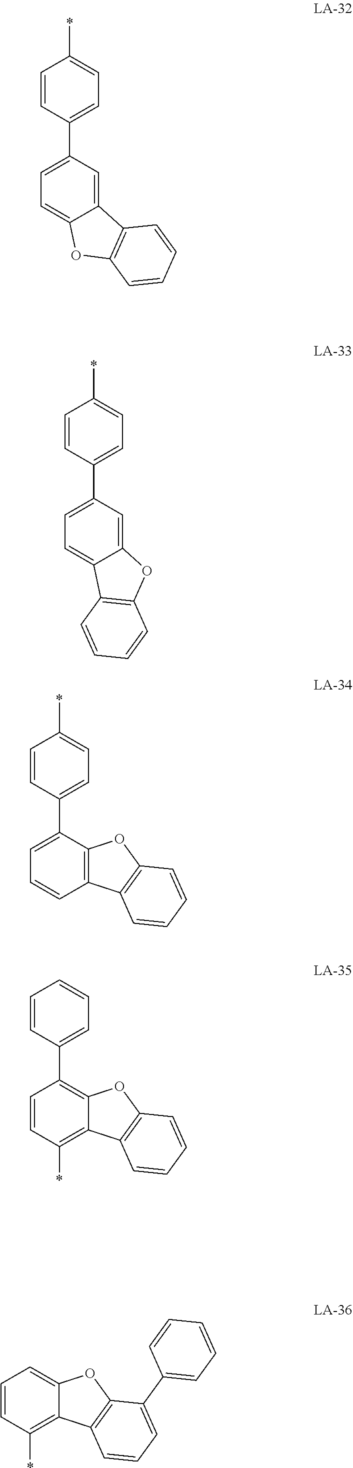 [SMILES: *c1ccc(-c2ccc3oc4ccccc4c3c2)cc1 ; *c1ccc(-c2ccc3c(c2)oc2ccccc23)cc1 ; *c1ccc(-c2cccc3c2oc2ccccc23)cc1 ; *c1ccc(-c2ccccc2)c2oc3ccccc3c12 ; *c1cccc2oc3c(-c4ccccc4)cccc3c12]

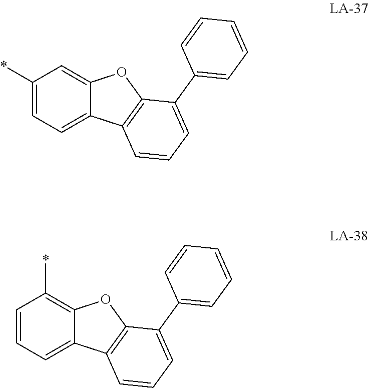 [SMILES: *c1ccc2c(c1)oc1c(-c3ccccc3)cccc12 ; *c1cccc2c1oc1c(-c3ccccc3)cccc12]

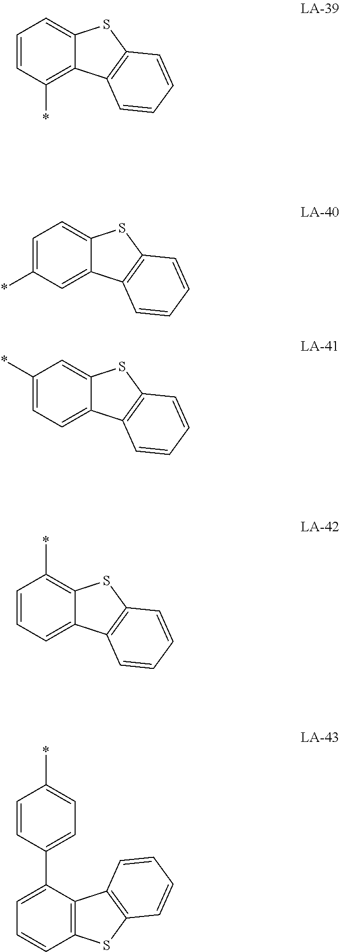 [SMILES: *c1cccc2sc3ccccc3c12 ; *c1ccc2sc3ccccc3c2c1 ; *c1ccc2c(c1)sc1ccccc12 ; *c1cccc2c1sc1ccccc12 ; *c1ccc(-c2cccc3sc4ccccc4c23)cc1]

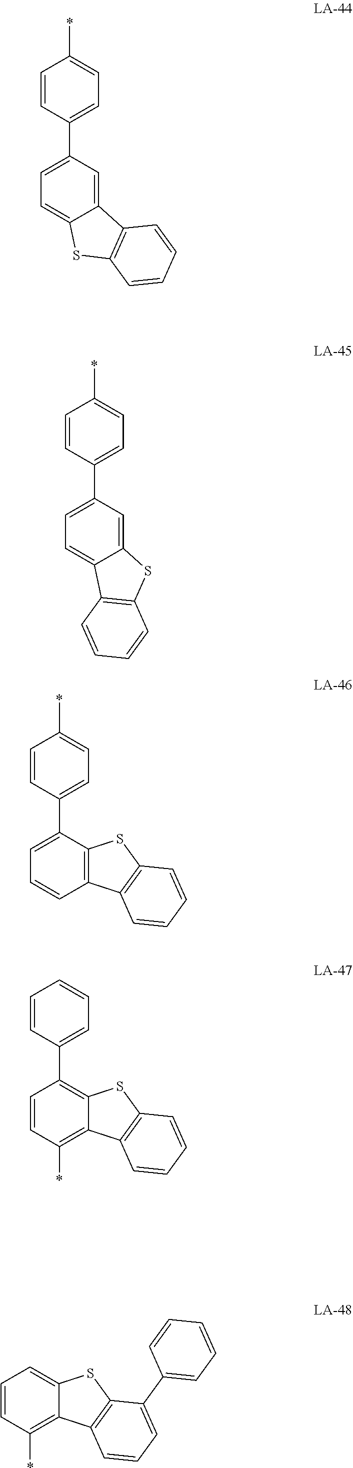 [SMILES: *c1ccc(-c2ccc3sc4ccccc4c3c2)cc1 ; *c1ccc(-c2ccc3c(c2)sc2ccccc23)cc1 ; *c1ccc(-c2cccc3c2sc2ccccc23)cc1 ; *c1ccc(-c2ccccc2)c2sc3ccccc3c12 ; *c1cccc2sc3c(-c4ccccc4)cccc3c12]

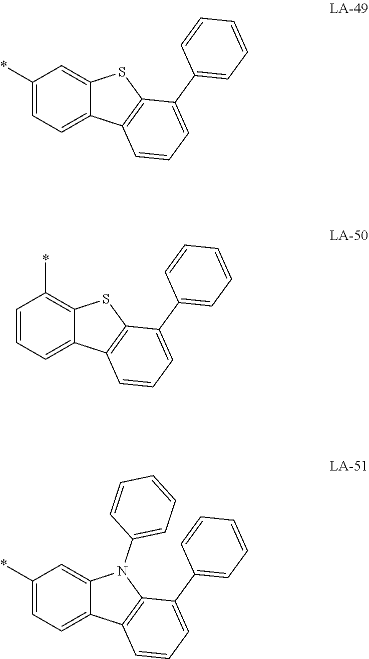 [SMILES: *c1ccc2c(c1)sc1c(-c3ccccc3)cccc12 ; *c1cccc2c1sc1c(-c3ccccc3)cccc12 ; *c1ccc2c3cccc(-c4ccccc4)c3n(-c3ccccc3)c2c1]

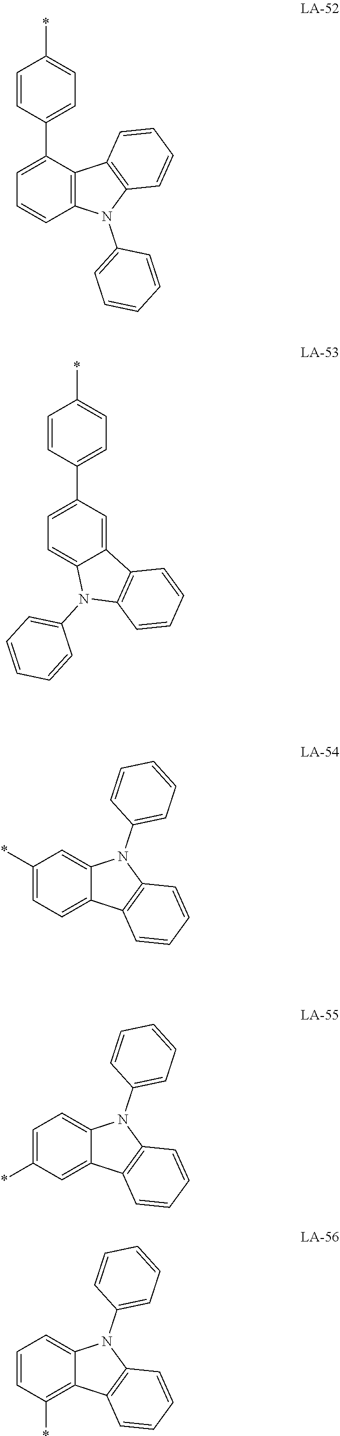

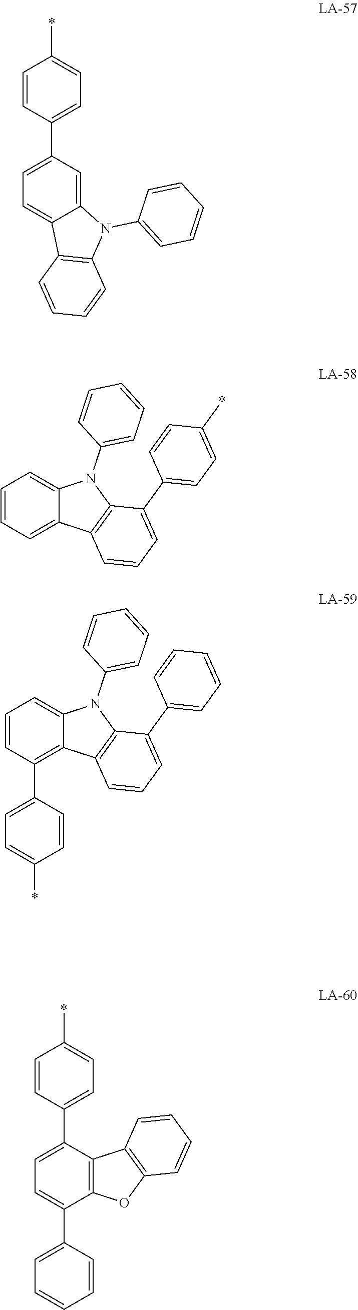

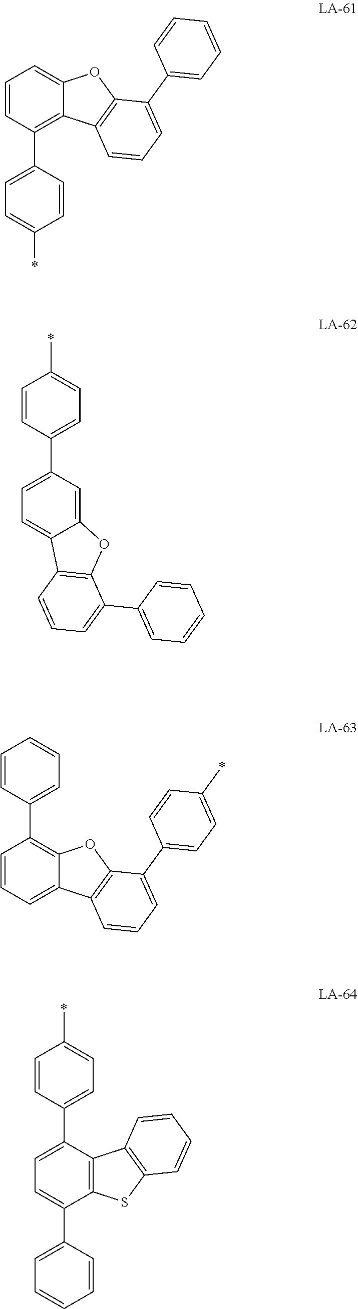 [SMILES: *c1ccc(-c2cccc3oc4c(-c5ccccc5)cccc4c23)cc1 ; *c1ccc(-c2ccc3c(c2)oc2c(-c4ccccc4)cccc23)cc1 ; *c1ccc(-c2cccc3c2oc2c(-c4ccccc4)cccc23)cc1 ; *c1ccc(-c2ccc(-c3ccccc3)c3sc4ccccc4c23)cc1]

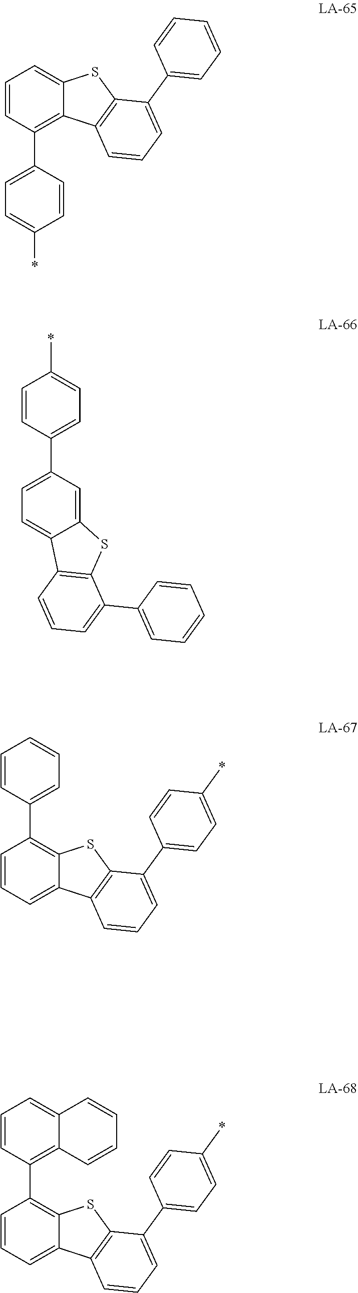 [SMILES: *c1ccc(-c2cccc3sc4c(-c5ccccc5)cccc4c23)cc1 ; *c1ccc(-c2ccc3c(c2)sc2c(-c4ccccc4)cccc23)cc1 ; *c1ccc(-c2cccc3c2sc2c(-c4ccccc4)cccc23)cc1 ; *c1ccc(-c2cccc3c2sc2c(-c4cccc5ccccc45)cccc23)cc1]

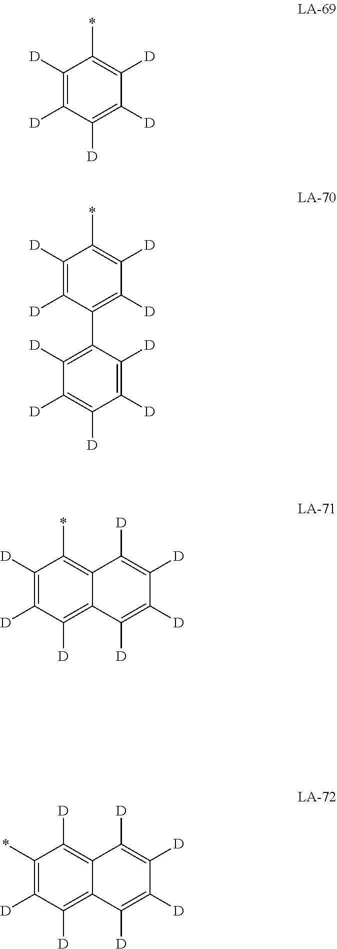 [SMILES: *c1c([2H])c([2H])c([2H])c([2H])c1[2H] ; *c1c([2H])c([2H])c(-c2c([2H])c([2H])c([2H])c([2H])c2[2H])c([2H])c1[2H] ; *c1c([2H])c([2H])c([2H])c2c([2H])c([2H])c([2H])c([2H])c12 ; *c1c([2H])c([2H])c2c([2H])c([2H])c([2H])c([2H])c2c1[2H]]

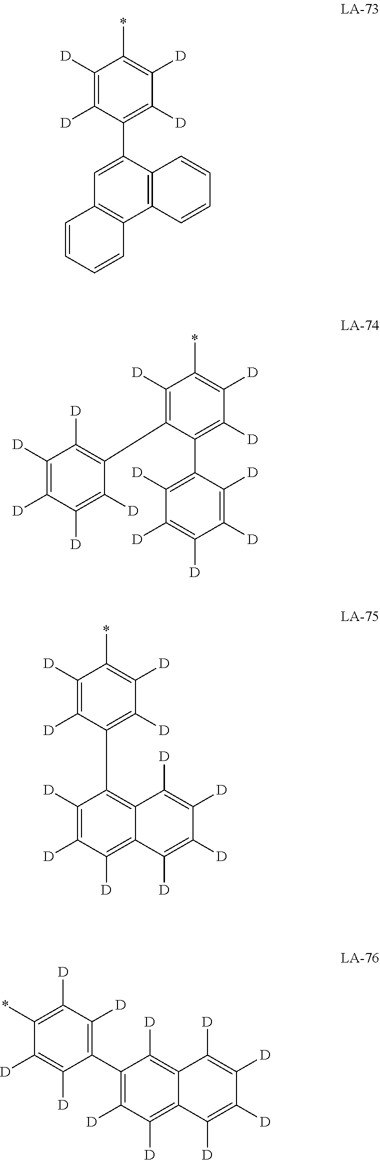 [SMILES: *c1c([2H])c([2H])c(-c2cc3ccccc3c3ccccc23)c([2H])c1[2H] ; *c1c([2H])c([2H])c(-c2c([2H])c([2H])c([2H])c([2H])c2[2H])c(-c2c([2H])c([2H])c([2H])c([2H])c2[2H])c1[2H] ; *c1c([2H])c([2H])c(-c2c([2H])c([2H])c([2H])c3c([2H])c([2H])c([2H])c([2H])c23)c([2H])c1[2H] ; *c1c([2H])c([2H])c(-c2c([2H])c([2H])c3c([2H])c([2H])c([2H])c([2H])c3c2[2H])c([2H])c1[2H]]

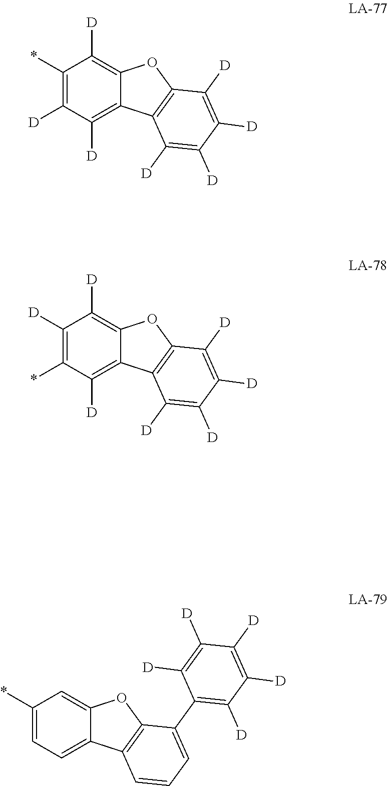 [SMILES: *c1c([2H])c([2H])c2c(oc3c([2H])c([2H])c([2H])c([2H])c32)c1[2H] ; *c1c([2H])c([2H])c2oc3c([2H])c([2H])c([2H])c([2H])c3c2c1[2H] ; *c1ccc2c(c1)oc1c(-c3c([2H])c([2H])c([2H])c([2H])c3[2H])cccc12]

In Formula 1, R1 to R6 may each independently be a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms. For example, R₁ to R₆ may each independently be a hydrogen atom, a deuterium atom, or a substituted or unsubstituted phenyl group. However, this is only an example, and embodiments are not limited thereto.

In Formula 1, Ar₂ may be a group represented by Formula 2. Formula 2 may represent a substituted or unsubstituted phenanthryl group. Ar₂ may correspond to the second substituent described above.

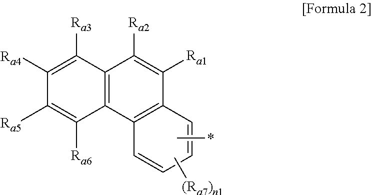

In Formula 2, n1 may be an integer from 0 to 3. In Formula 2, Rₐ₁ to Rₐ₇ may each independently be a hydrogen atom, a deuterium atom, a substituted or unsubstituted alkyl group having 1 to 10 carbon atoms, a substituted or unsubstituted aryl group having 6 to 15 ring-forming carbon atoms, or substituted or unsubstituted heteroaryl group having 2 to 15 ring-forming carbon atoms. In an embodiment, Rₐ₁ to Rₐ₇ may each independently be a hydrogen atom or a deuterium atom. In Formula 2, [symbol] represents a bond to a neighboring atom. For example, [symbol] may represent a bond to N in Formula 1.

If n1 is 2 or more, multiple Rₐ₇ groups may be the same as each other or at least one thereof may be different from the remainder. A case where n1 is 0 may be the same as a case where n1 is 3 and three Rₐy groups are all hydrogen atoms.

In an embodiment, the amine compound represented by Formula 1 may be represented by Formula 1-A1 or Formula 1-A2. Formula 1-A1 may represent a case where X₁ is O in Formula 1. Formula 1-A2 may represent a case where X₁ is S in Formula 1.

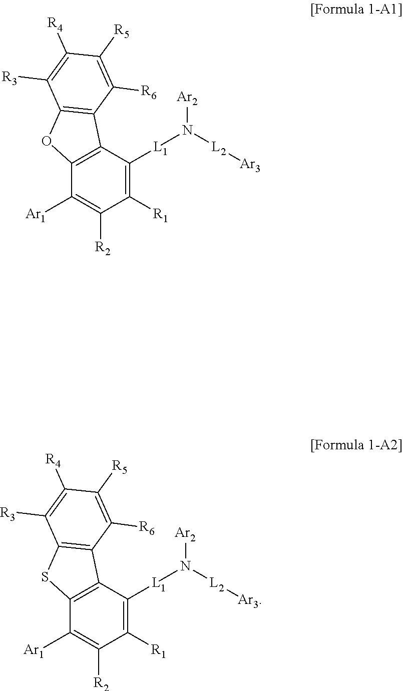

In Formulas 1-A1 and 1-A2, L₁, L₂, Aᵣ₁, Ar₂, Ar₃, and R₁ to R₆ are the same as defined in Formula 1.

In an embodiment, the amine compound represented by Formula 1 may be represented by one of Formulas 1-B1 to 1-B4. Formulas 1-B1 to 1-B4 each represent a case where the bonding position of the group represented by Formula 2 to Formula 1 is further defined.

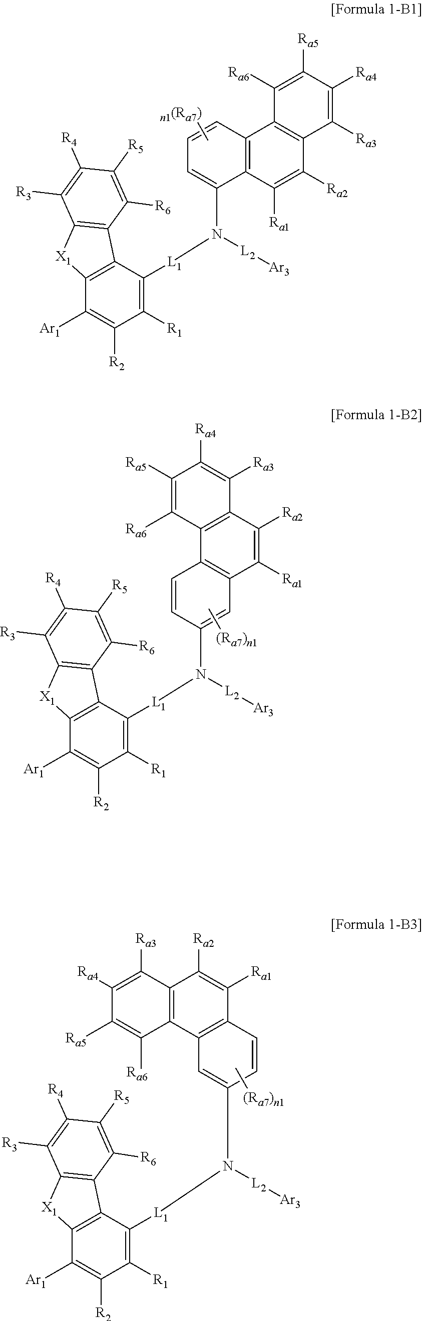

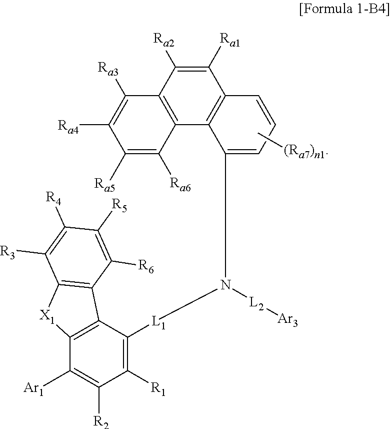

In Formulas 1-B1 to 1-B4, L₁, L₂, Aᵣ₁, Ar₂, Ar₃, R₁ to R₆, Rₐ₁ to Rₐ₇, and n1 are the same as defined in Formulas 1 and 2.

In an embodiment, in Formulas 1-B1 to 1-B4, Rₐ₁ to Rₐ₇ may each independently be a hydrogen atom or a deuterium atom.

In an embodiment, the amine compound may be any compound selected from Compound Group 1. In an embodiment, in the light-emitting element ED, the amine compound may include at least one compound selected from Compound Group 1:PG-4e,

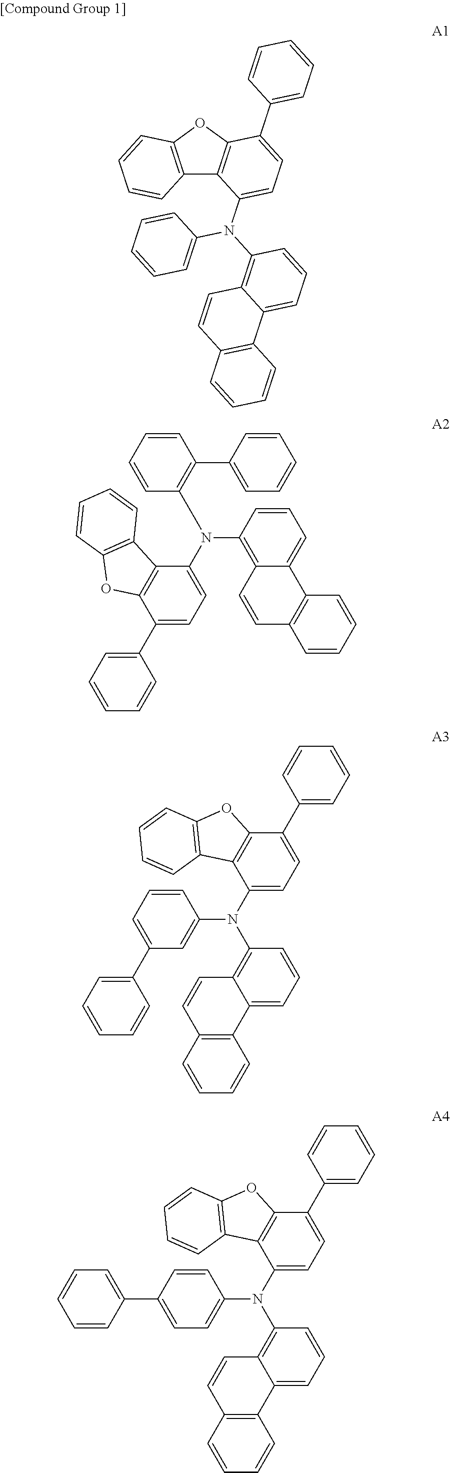

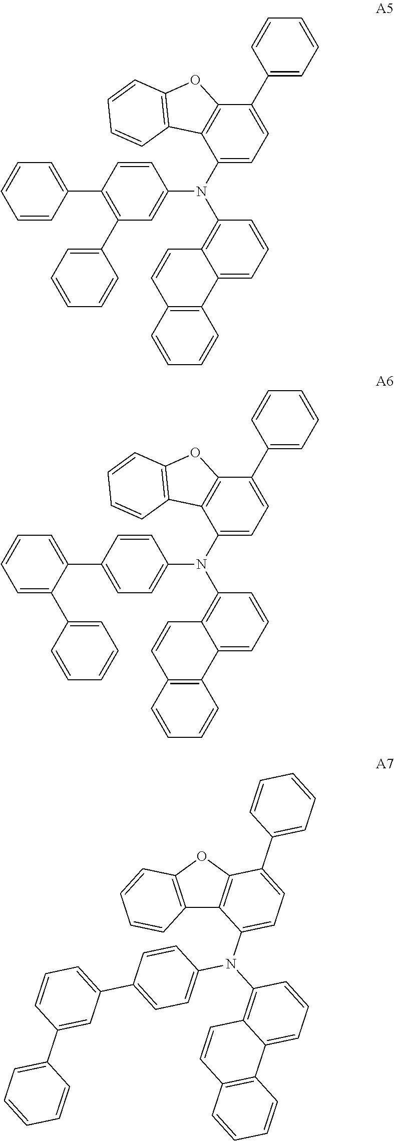 [SMILES: c1ccc(-c2ccc(N(c3cccc4c3ccc3ccccc34)c3ccc(-c4ccccc4)c4oc5ccccc5c34)cc2-c2ccccc2)cc1 ; c1ccc(-c2ccccc2-c2ccc(N(c3cccc4c3ccc3ccccc34)c3ccc(-c4ccccc4)c4oc5ccccc5c34)cc2)cc1 ; c1ccc(-c2cccc(-c3ccc(N(c4cccc5c4ccc4ccccc45)c4ccc(-c5ccccc5)c5oc6ccccc6c45)cc3)c2)cc1]

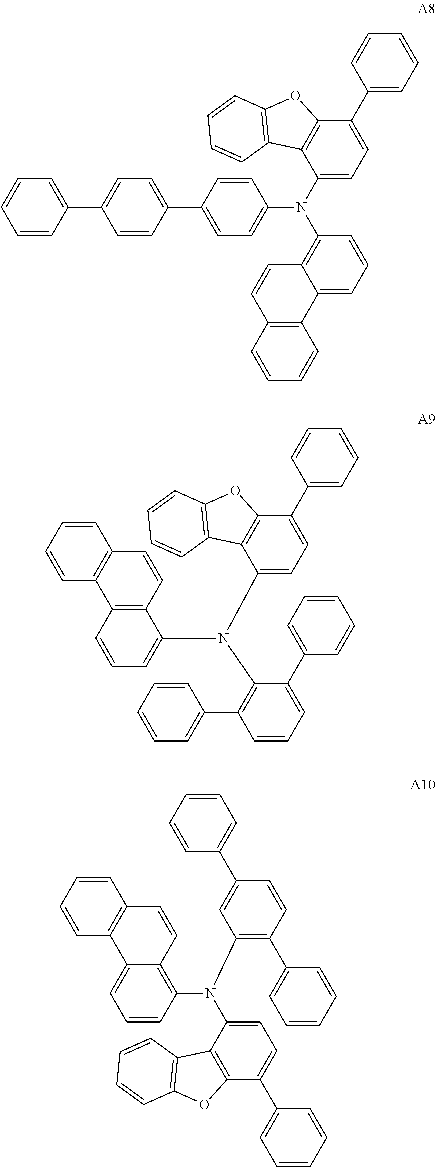 [SMILES: c1ccc(-c2ccc(-c3ccc(N(c4cccc5c4ccc4ccccc45)c4ccc(-c5ccccc5)c5oc6ccccc6c45)cc3)cc2)cc1 ; c1ccc(-c2cccc(-c3ccccc3)c2N(c2cccc3c2ccc2ccccc23)c2ccc(-c3ccccc3)c3oc4ccccc4c23)cc1 ; c1ccc(-c2ccc(-c3ccccc3)c(N(c3cccc4c3ccc3ccccc34)c3ccc(-c4ccccc4)c4oc5ccccc5c34)c2)cc1]

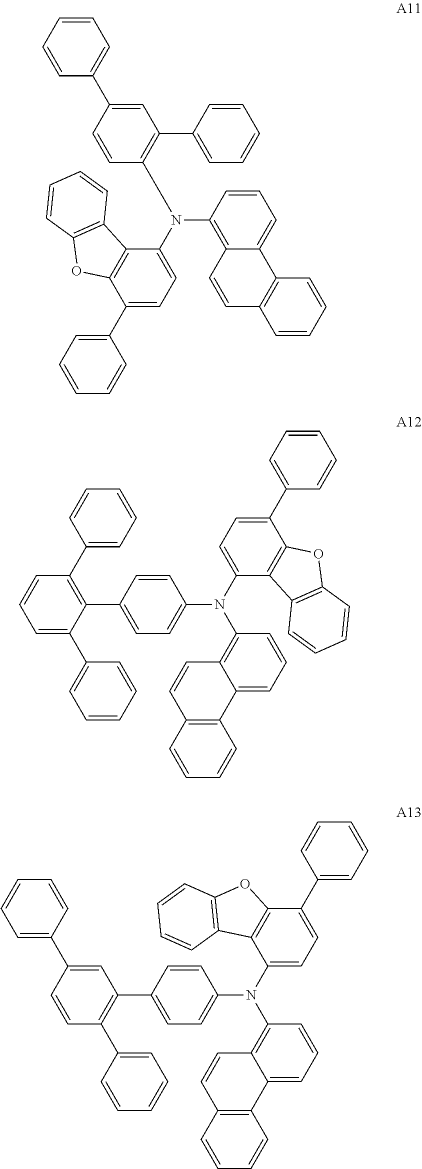 [SMILES: c1ccc(-c2ccc(N(c3cccc4c3ccc3ccccc34)c3ccc(-c4ccccc4)c4oc5ccccc5c34)c(-c3ccccc3)c2)cc1 ; c1ccc(-c2cccc(-c3ccccc3)c2-c2ccc(N(c3cccc4c3ccc3ccccc34)c3ccc(-c4ccccc4)c4oc5ccccc5c34)cc2)cc1 ; c1ccc(-c2ccc(-c3ccccc3)c(-c3ccc(N(c4cccc5c4ccc4ccccc45)c4ccc(-c5ccccc5)c5oc6ccccc6c45)cc3)c2)cc1]

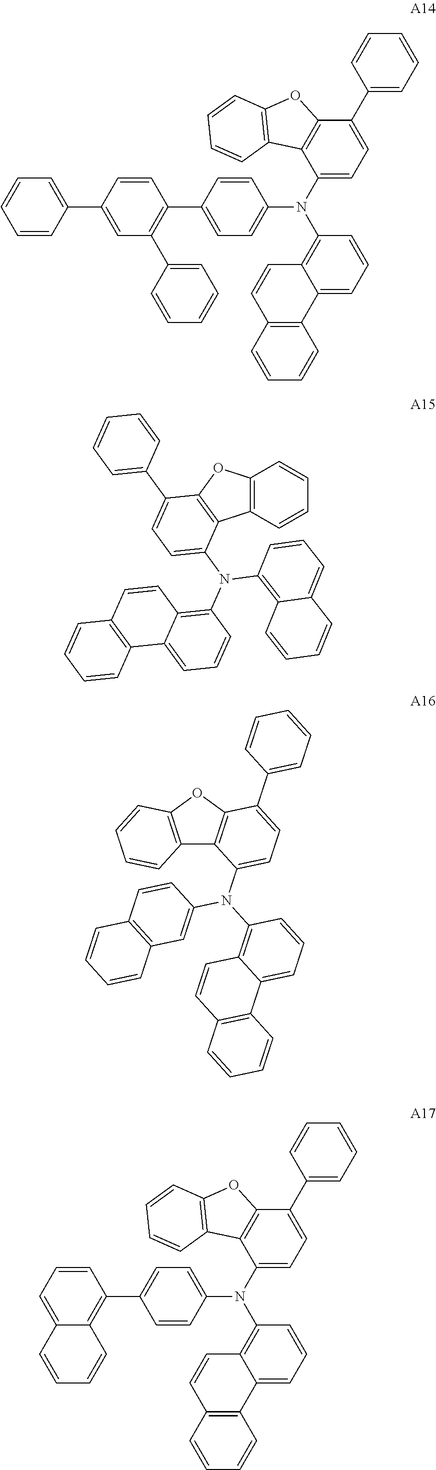 [SMILES: c1ccc(-c2ccc(-c3ccc(N(c4cccc5c4ccc4ccccc45)c4ccc(-c5ccccc5)c5oc6ccccc6c45)cc3)c(-c3ccccc3)c2)cc1 ; c1ccc(-c2ccc(N(c3cccc4ccccc34)c3cccc4c3ccc3ccccc34)c3c2oc2ccccc23)cc1 ; c1ccc(-c2ccc(N(c3ccc4ccccc4c3)c3cccc4c3ccc3ccccc34)c3c2oc2ccccc23)cc1 ; c1ccc(-c2ccc(N(c3ccc(-c4cccc5ccccc45)cc3)c3cccc4c3ccc3ccccc34)c3c2oc2ccccc23)cc1]

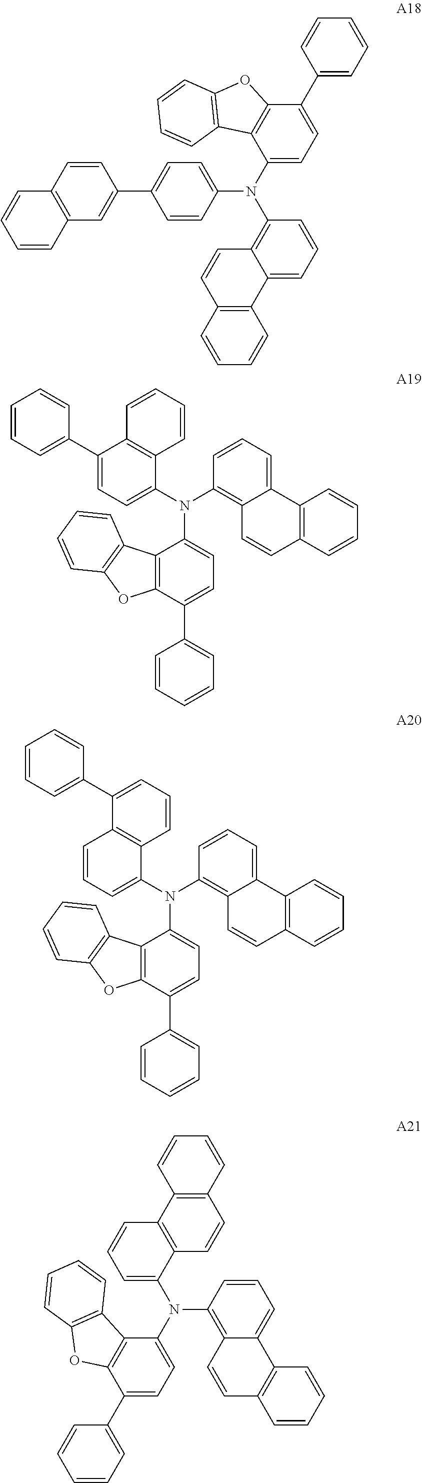

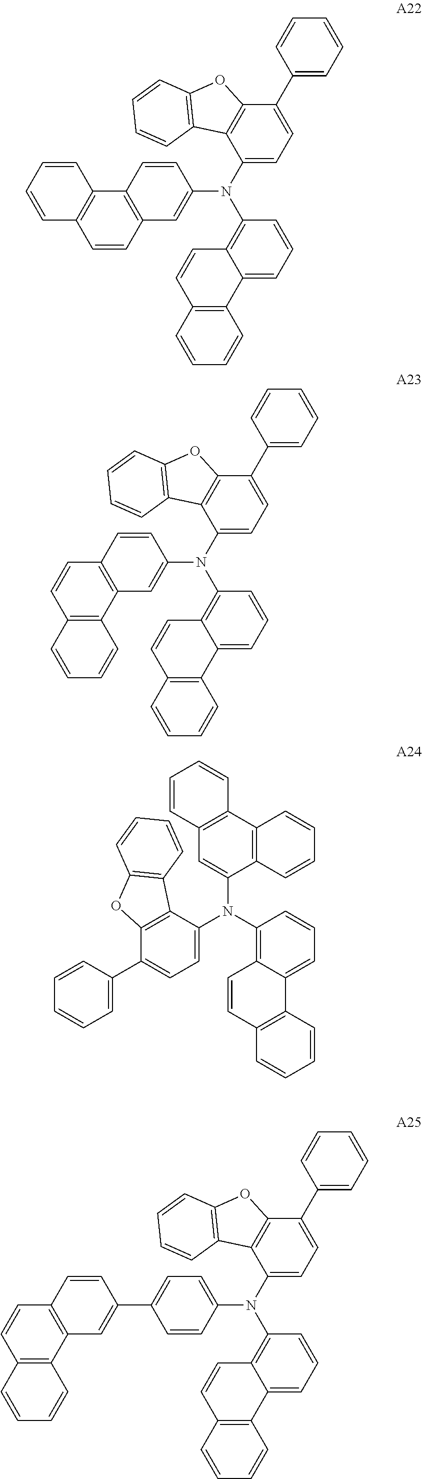

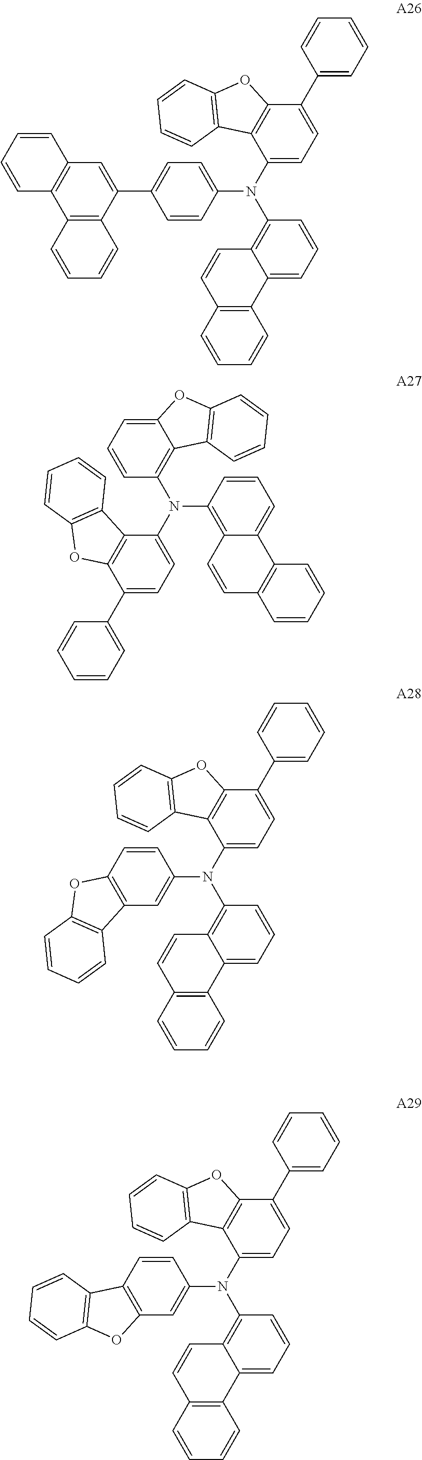

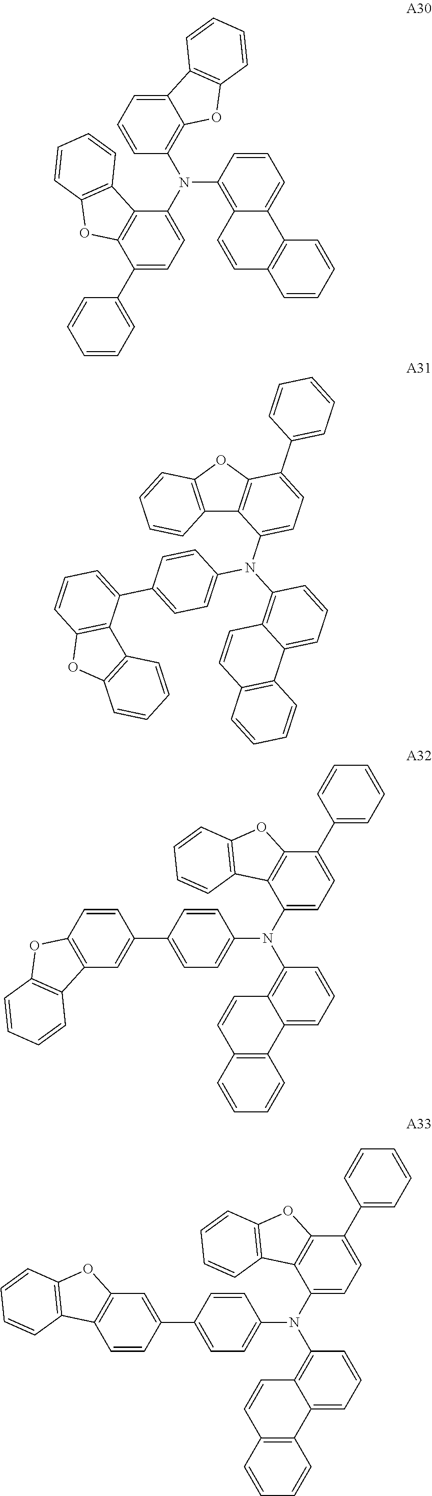 [SMILES: c1ccc(-c2ccc(N(c3cccc4c3ccc3ccccc34)c3cccc4c3oc3ccccc34)c3c2oc2ccccc23)cc1 ; c1ccc(-c2ccc(N(c3ccc(-c4cccc5oc6ccccc6c45)cc3)c3cccc4c3ccc3ccccc34)c3c2oc2ccccc23)cc1 ; c1ccc(-c2ccc(N(c3ccc(-c4ccc5oc6ccccc6c5c4)cc3)c3cccc4c3ccc3ccccc34)c3c2oc2ccccc23)cc1 ; c1ccc(-c2ccc(N(c3ccc(-c4ccc5c(c4)oc4ccccc45)cc3)c3cccc4c3ccc3ccccc34)c3c2oc2ccccc23)cc1]

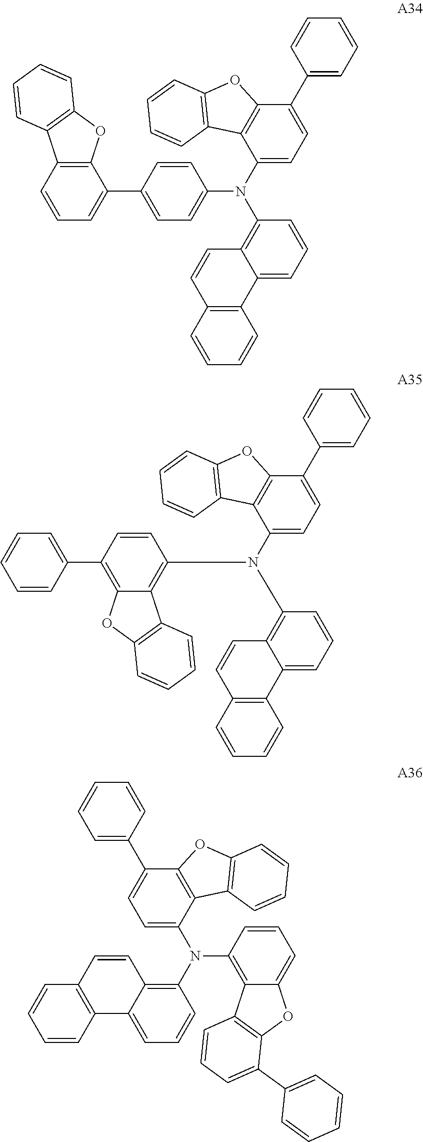 [SMILES: c1ccc(-c2ccc(N(c3ccc(-c4cccc5c4oc4ccccc45)cc3)c3cccc4c3ccc3ccccc34)c3c2oc2ccccc23)cc1 ; c1ccc(-c2ccc(N(c3cccc4c3ccc3ccccc34)c3ccc(-c4ccccc4)c4oc5ccccc5c34)c3c2oc2ccccc23)cc1 ; c1ccc(-c2cccc3c2oc2cccc(N(c4cccc5c4ccc4ccccc45)c4ccc(-c5ccccc5)c5oc6ccccc6c45)c23)cc1]

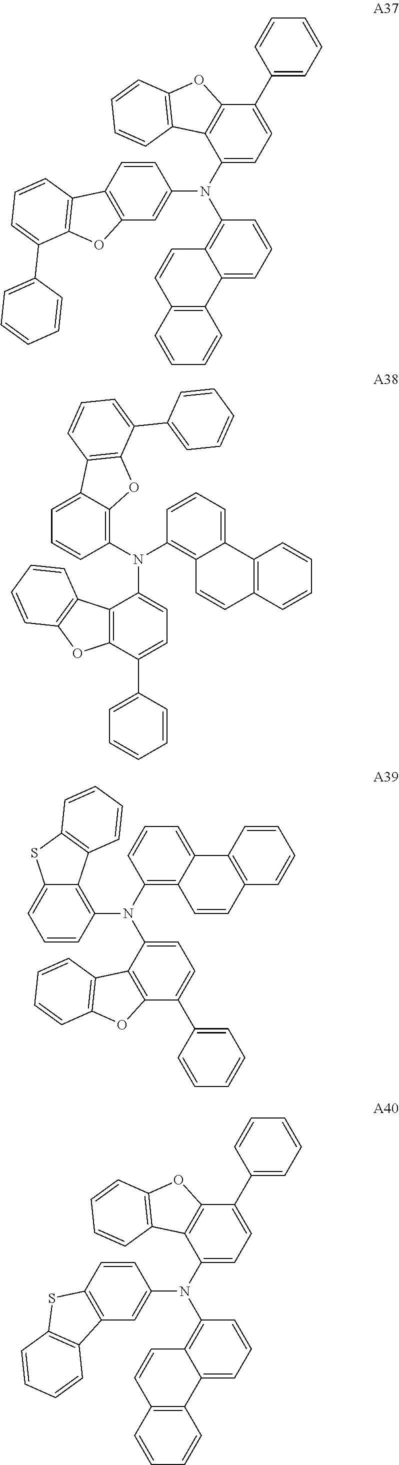

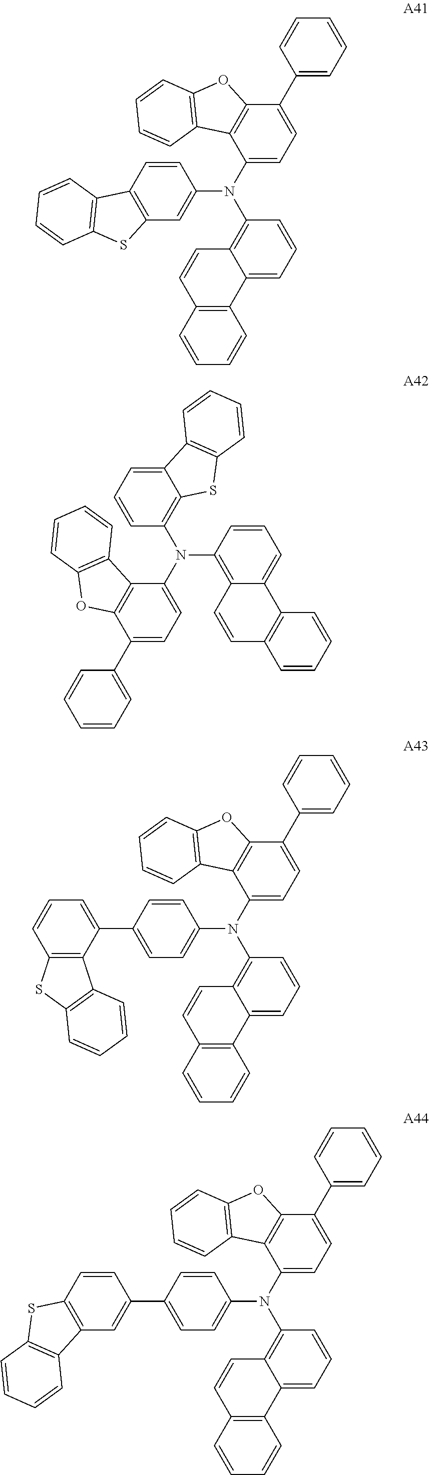

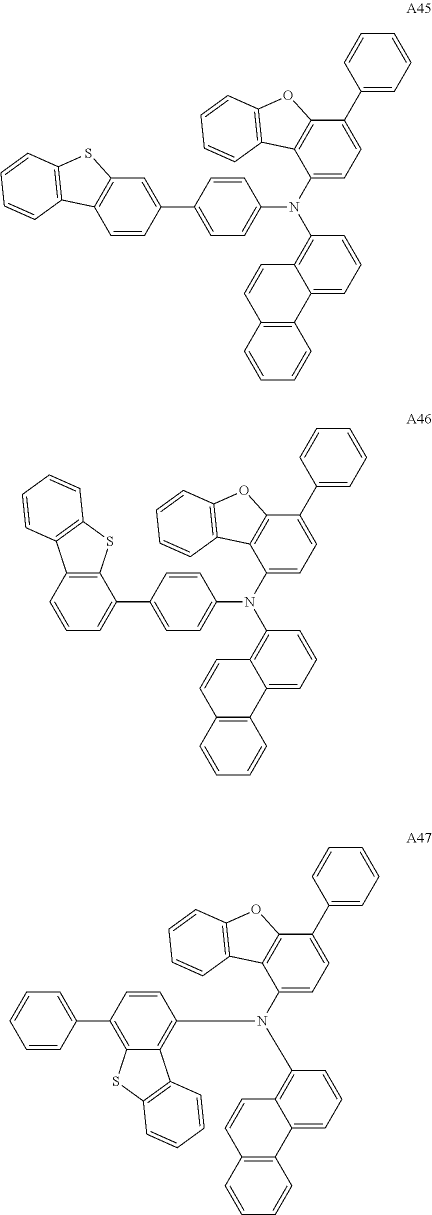 [SMILES: c1ccc(-c2ccc(N(c3ccc(-c4ccc5c(c4)sc4ccccc45)cc3)c3cccc4c3ccc3ccccc34)c3c2oc2ccccc23)cc1 ; c1ccc(-c2ccc(N(c3ccc(-c4cccc5c4sc4ccccc45)cc3)c3cccc4c3ccc3ccccc34)c3c2oc2ccccc23)cc1 ; c1ccc(-c2ccc(N(c3cccc4c3ccc3ccccc34)c3ccc(-c4ccccc4)c4sc5ccccc5c34)c3c2oc2ccccc23)cc1]

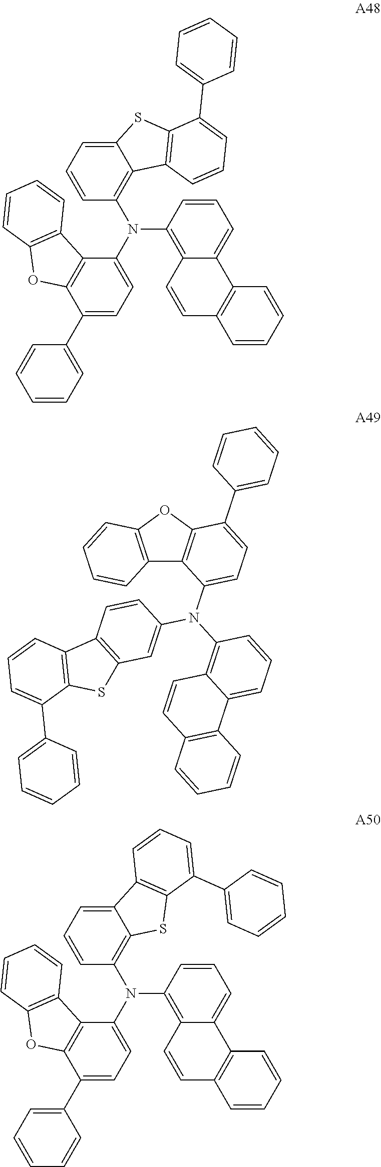 [SMILES: c1ccc(-c2ccc(N(c3cccc4c3ccc3ccccc34)c3cccc4sc5c(-c6ccccc6)cccc5c34)c3c2oc2ccccc23)cc1 ; c1ccc(-c2ccc(N(c3ccc4c(c3)sc3c(-c5ccccc5)cccc34)c3cccc4c3ccc3ccccc34)c3c2oc2ccccc23)cc1 ; c1ccc(-c2ccc(N(c3cccc4c3ccc3ccccc34)c3cccc4c3sc3c(-c5ccccc5)cccc34)c3c2oc2ccccc23)cc1]

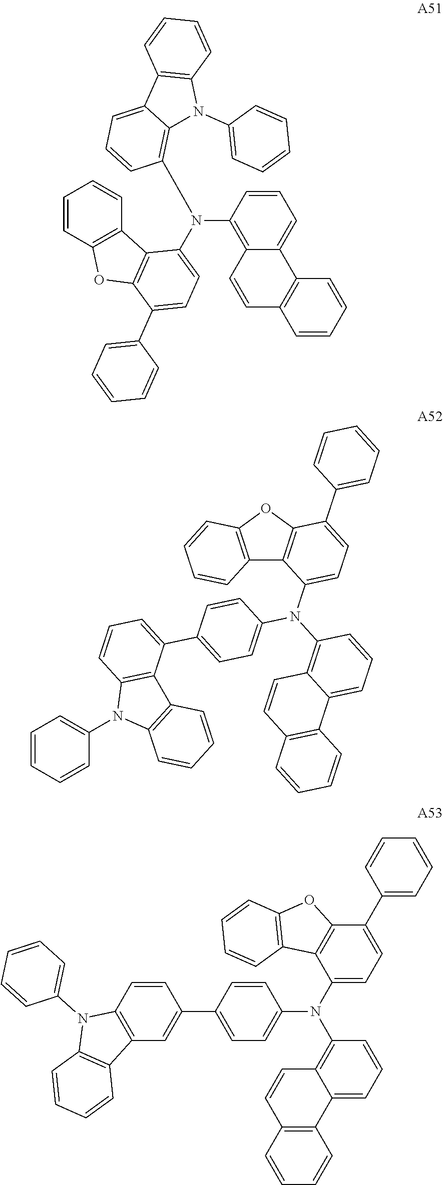 [SMILES: c1ccc(-c2ccc(N(c3cccc4c3ccc3ccccc34)c3cccc4c5ccccc5n(-c5ccccc5)c34)c3c2oc2ccccc23)cc1 ; c1ccc(-c2ccc(N(c3ccc(-c4cccc5c4c4ccccc4n5-c4ccccc4)cc3)c3cccc4c3ccc3ccccc34)c3c2oc2ccccc23)cc1 ; c1ccc(-c2ccc(N(c3ccc(-c4ccc5c(c4)c4ccccc4n5-c4ccccc4)cc3)c3cccc4c3ccc3ccccc34)c3c2oc2ccccc23)cc1]

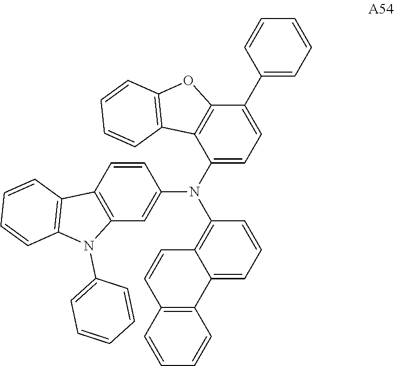 [SMILES: c1ccc(-c2ccc(N(c3ccc4c5ccccc5n(-c5ccccc5)c4c3)c3cccc4c3ccc3ccccc34)c3c2oc2ccccc23)cc1]

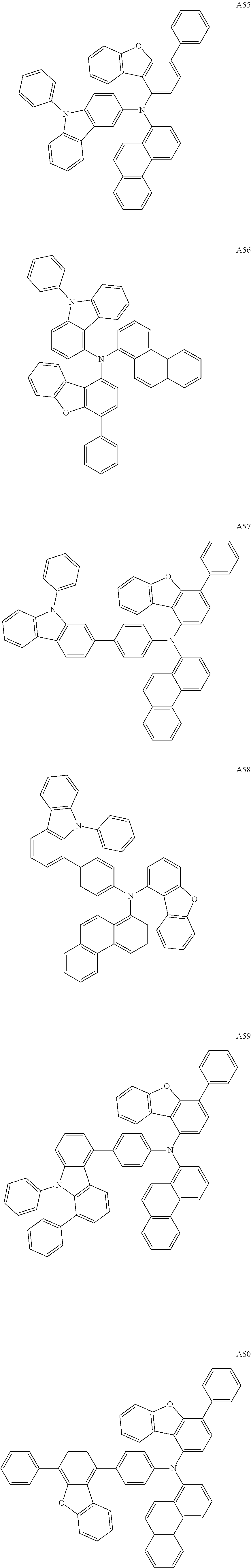

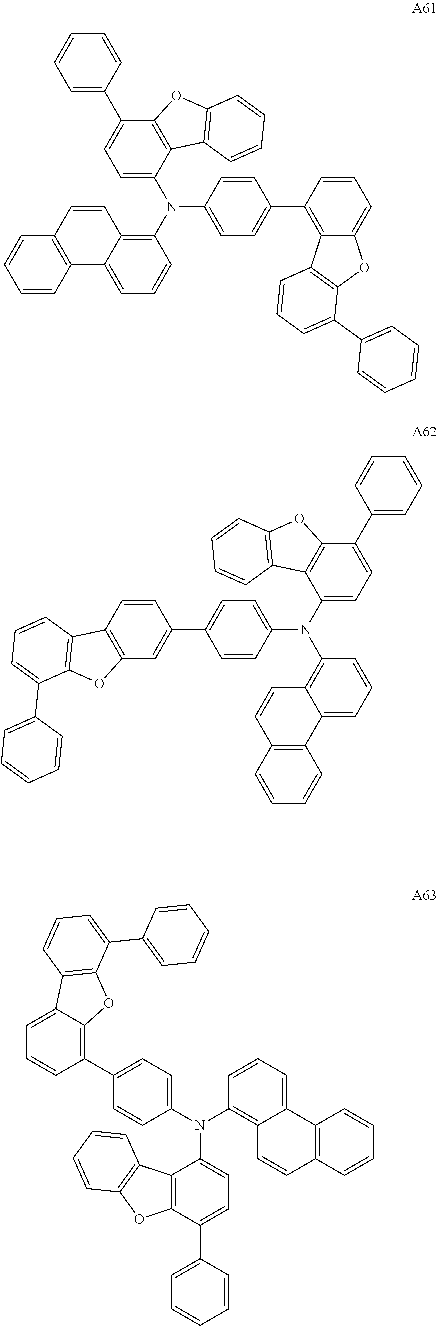 [SMILES: c1ccc(-c2cccc3c2oc2cccc(-c4ccc(N(c5cccc6c5ccc5ccccc56)c5ccc(-c6ccccc6)c6oc7ccccc7c56)cc4)c23)cc1 ; c1ccc(-c2cccc3c2oc2cc(-c4ccc(N(c5cccc6c5ccc5ccccc56)c5ccc(-c6ccccc6)c6oc7ccccc7c56)cc4)ccc23)cc1 ; c1ccc(-c2cccc3c2oc2c(-c4ccc(N(c5cccc6c5ccc5ccccc56)c5ccc(-c6ccccc6)c6oc7ccccc7c56)cc4)cccc23)cc1]

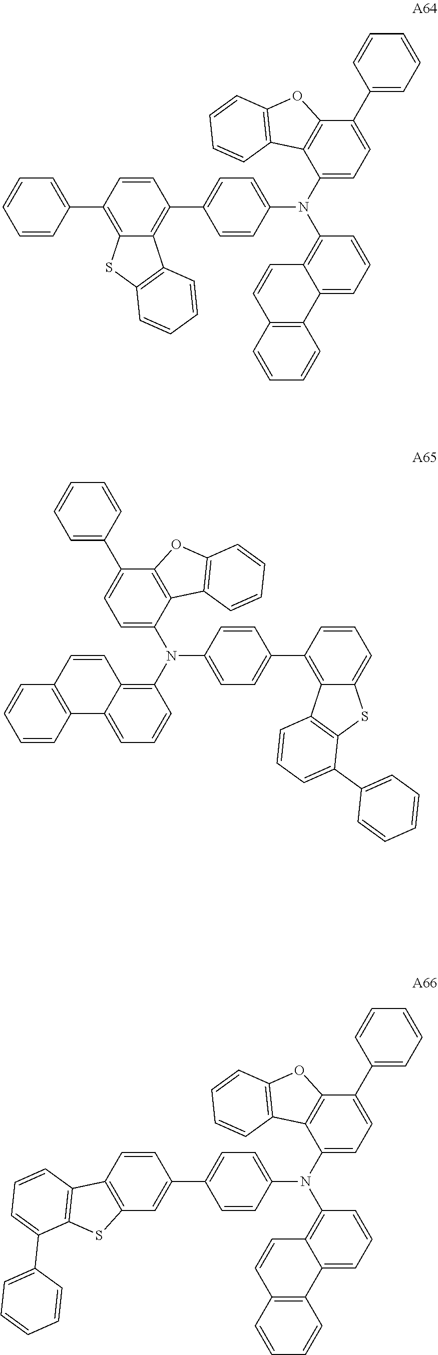 [SMILES: c1ccc(-c2ccc(N(c3ccc(-c4ccc(-c5ccccc5)c5sc6ccccc6c45)cc3)c3cccc4c3ccc3ccccc34)c3c2oc2ccccc23)cc1 ; c1ccc(-c2ccc(N(c3ccc(-c4cccc5sc6c(-c7ccccc7)cccc6c45)cc3)c3cccc4c3ccc3ccccc34)c3c2oc2ccccc23)cc1 ; c1ccc(-c2ccc(N(c3ccc(-c4ccc5c(c4)sc4c(-c6ccccc6)cccc45)cc3)c3cccc4c3ccc3ccccc34)c3c2oc2ccccc23)cc1]

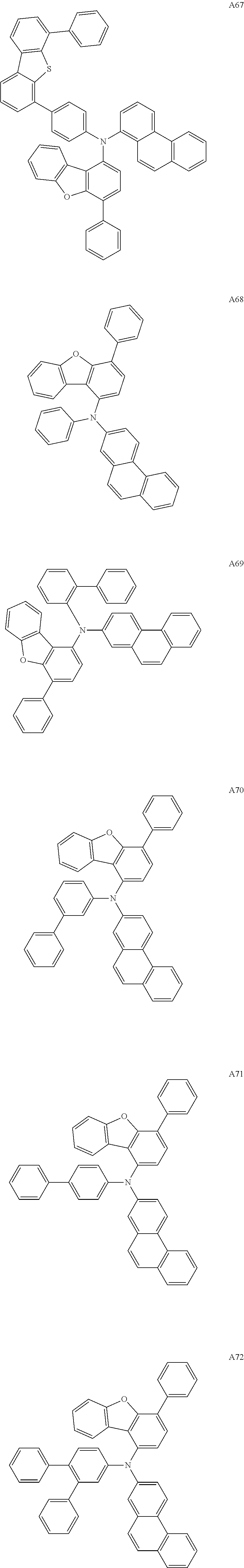 [SMILES: c1ccc(-c2ccc(N(c3ccc(-c4cccc5c4sc4c(-c6ccccc6)cccc45)cc3)c3cccc4c3ccc3ccccc34)c3c2oc2ccccc23)cc1 ; c1ccc(-c2ccc(N(c3ccccc3)c3ccc4c(ccc5ccccc54)c3)c3c2oc2ccccc23)cc1 ; c1ccc(-c2ccccc2N(c2ccc3c(ccc4ccccc43)c2)c2ccc(-c3ccccc3)c3oc4ccccc4c23)cc1 ; c1ccc(-c2cccc(N(c3ccc4c(ccc5ccccc54)c3)c3ccc(-c4ccccc4)c4oc5ccccc5c34)c2)cc1 ; c1ccc(-c2ccc(N(c3ccc4c(ccc5ccccc54)c3)c3ccc(-c4ccccc4)c4oc5ccccc5c34)cc2)cc1 ; c1ccc(-c2ccc(N(c3ccc4c(ccc5ccccc54)c3)c3ccc(-c4ccccc4)c4oc5ccccc5c34)cc2-c2ccccc2)cc1]

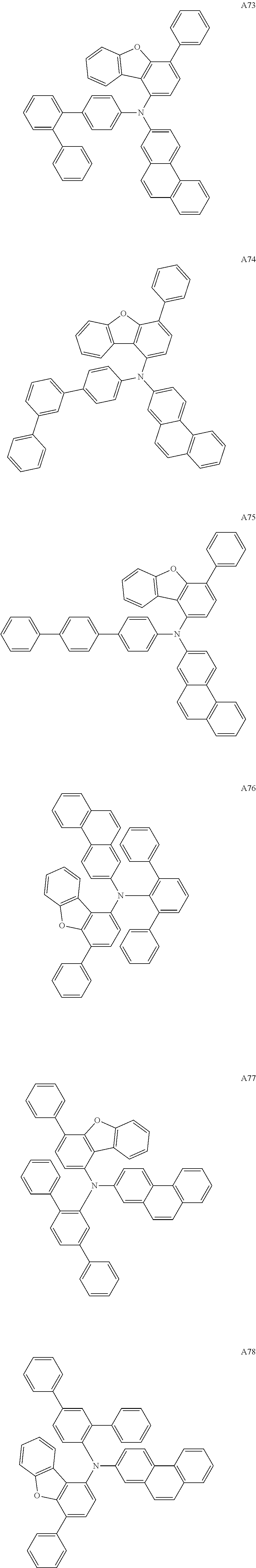

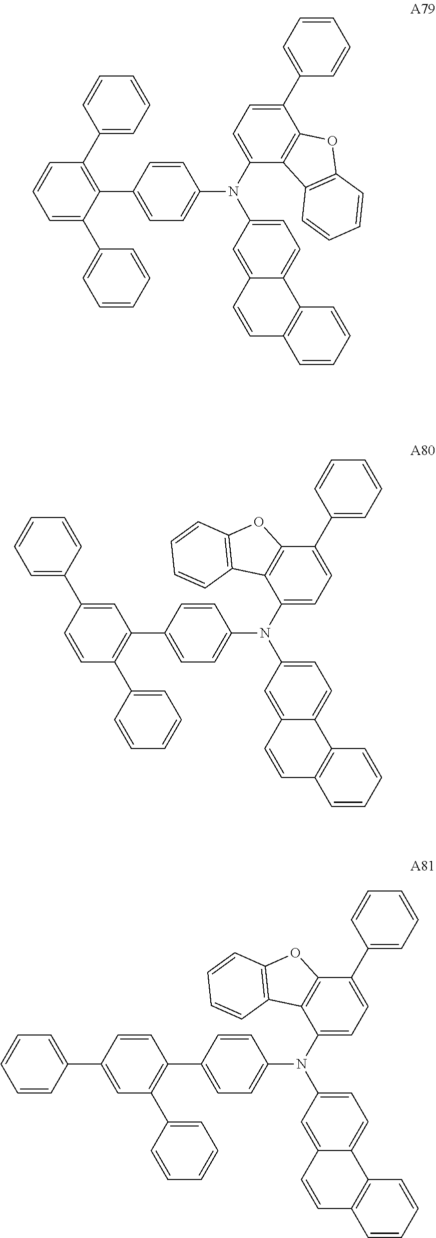 [SMILES: c1ccc(-c2cccc(-c3ccccc3)c2-c2ccc(N(c3ccc4c(ccc5ccccc54)c3)c3ccc(-c4ccccc4)c4oc5ccccc5c34)cc2)cc1 ; c1ccc(-c2ccc(-c3ccccc3)c(-c3ccc(N(c4ccc5c(ccc6ccccc65)c4)c4ccc(-c5ccccc5)c5oc6ccccc6c45)cc3)c2)cc1 ; c1ccc(-c2ccc(-c3ccc(N(c4ccc5c(ccc6ccccc65)c4)c4ccc(-c5ccccc5)c5oc6ccccc6c45)cc3)c(-c3ccccc3)c2)cc1]

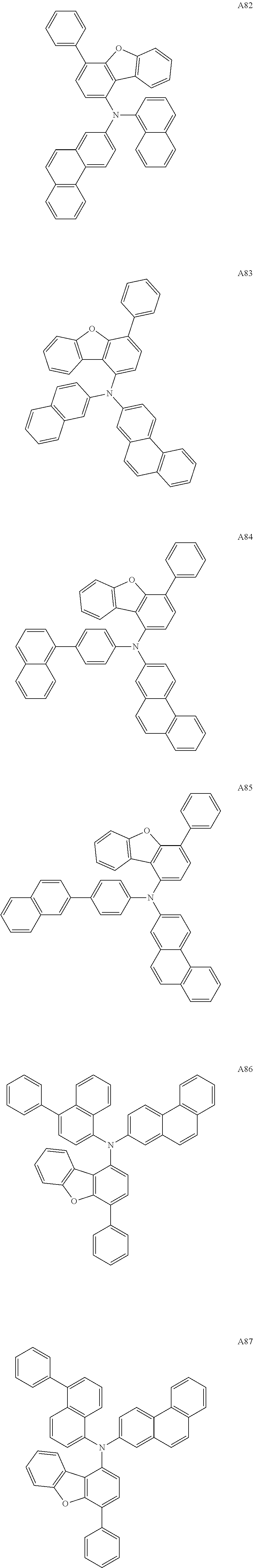 [SMILES: c1ccc(-c2ccc(N(c3ccc4c(ccc5ccccc54)c3)c3cccc4ccccc34)c3c2oc2ccccc23)cc1 ; c1ccc(-c2ccc(N(c3ccc4ccccc4c3)c3ccc4c(ccc5ccccc54)c3)c3c2oc2ccccc23)cc1 ; c1ccc(-c2ccc(N(c3ccc(-c4cccc5ccccc45)cc3)c3ccc4c(ccc5ccccc54)c3)c3c2oc2ccccc23)cc1 ; c1ccc(-c2ccc(N(c3ccc(-c4ccc5ccccc5c4)cc3)c3ccc4c(ccc5ccccc54)c3)c3c2oc2ccccc23)cc1 ; c1ccc(-c2ccc(N(c3ccc4c(ccc5ccccc54)c3)c3ccc(-c4ccccc4)c4oc5ccccc5c34)c3ccccc23)cc1 ; c1ccc(-c2cccc3c(N(c4ccc5c(ccc6ccccc65)c4)c4ccc(-c5ccccc5)c5oc6ccccc6c45)cccc23)cc1]

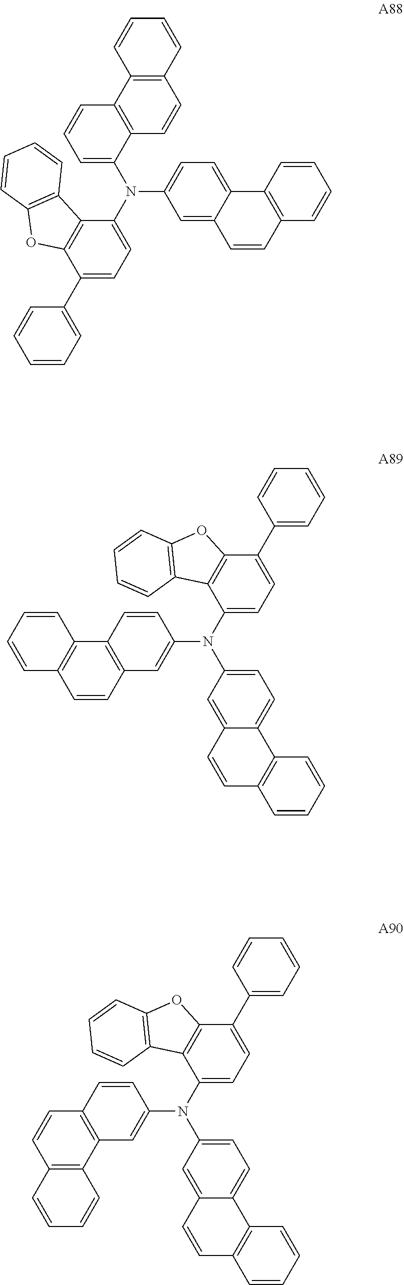 [SMILES: c1ccc(-c2ccc(N(c3ccc4c(ccc5ccccc54)c3)c3cccc4c3ccc3ccccc34)c3c2oc2ccccc23)cc1 ; c1ccc(-c2ccc(N(c3ccc4c(ccc5ccccc54)c3)c3ccc4c(ccc5ccccc54)c3)c3c2oc2ccccc23)cc1 ; c1ccc(-c2ccc(N(c3ccc4c(ccc5ccccc54)c3)c3ccc4ccc5ccccc5c4c3)c3c2oc2ccccc23)cc1]

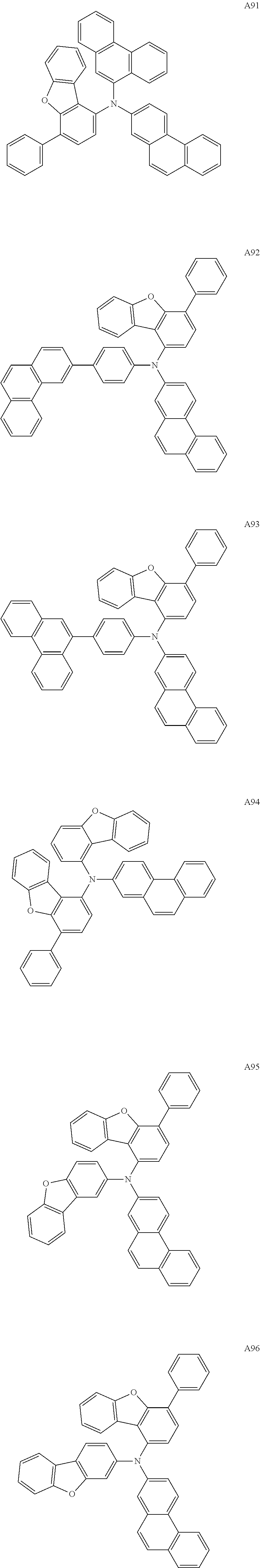 [SMILES: c1ccc(-c2ccc(N(c3ccc4c(ccc5ccccc54)c3)c3cc4ccccc4c4ccccc34)c3c2oc2ccccc23)cc1 ; c1ccc(-c2ccc(N(c3ccc(-c4ccc5ccc6ccccc6c5c4)cc3)c3ccc4c(ccc5ccccc54)c3)c3c2oc2ccccc23)cc1 ; c1ccc(-c2ccc(N(c3ccc(-c4cc5ccccc5c5ccccc45)cc3)c3ccc4c(ccc5ccccc54)c3)c3c2oc2ccccc23)cc1 ; c1ccc(-c2ccc(N(c3ccc4c(ccc5ccccc54)c3)c3cccc4oc5ccccc5c34)c3c2oc2ccccc23)cc1 ; c1ccc(-c2ccc(N(c3ccc4c(ccc5ccccc54)c3)c3ccc4oc5ccccc5c4c3)c3c2oc2ccccc23)cc1 ; c1ccc(-c2ccc(N(c3ccc4c(ccc5ccccc54)c3)c3ccc4c(c3)oc3ccccc34)c3c2oc2ccccc23)cc1]

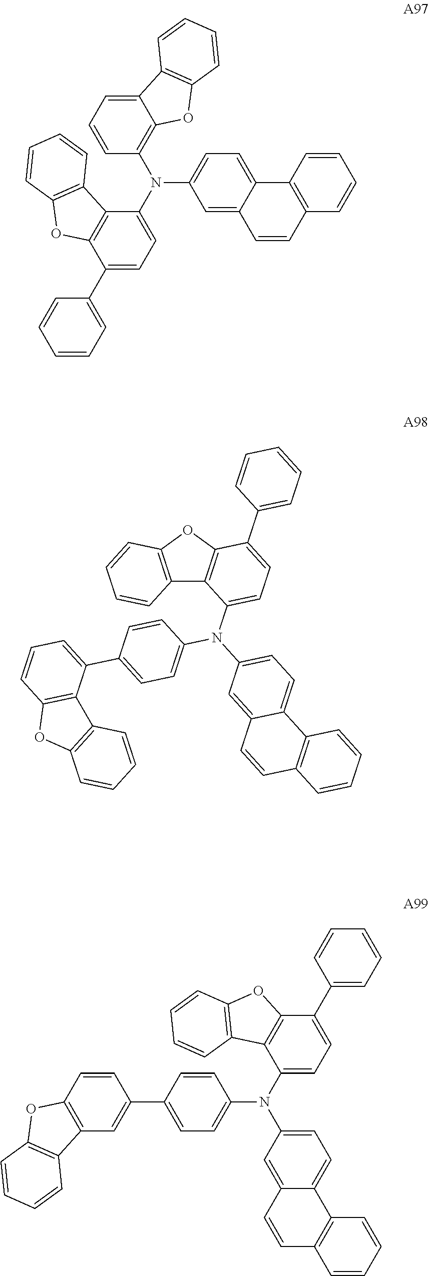 [SMILES: c1ccc(-c2ccc(N(c3ccc4c(ccc5ccccc54)c3)c3cccc4c3oc3ccccc34)c3c2oc2ccccc23)cc1 ; c1ccc(-c2ccc(N(c3ccc(-c4cccc5oc6ccccc6c45)cc3)c3ccc4c(ccc5ccccc54)c3)c3c2oc2ccccc23)cc1 ; c1ccc(-c2ccc(N(c3ccc(-c4ccc5oc6ccccc6c5c4)cc3)c3ccc4c(ccc5ccccc54)c3)c3c2oc2ccccc23)cc1]

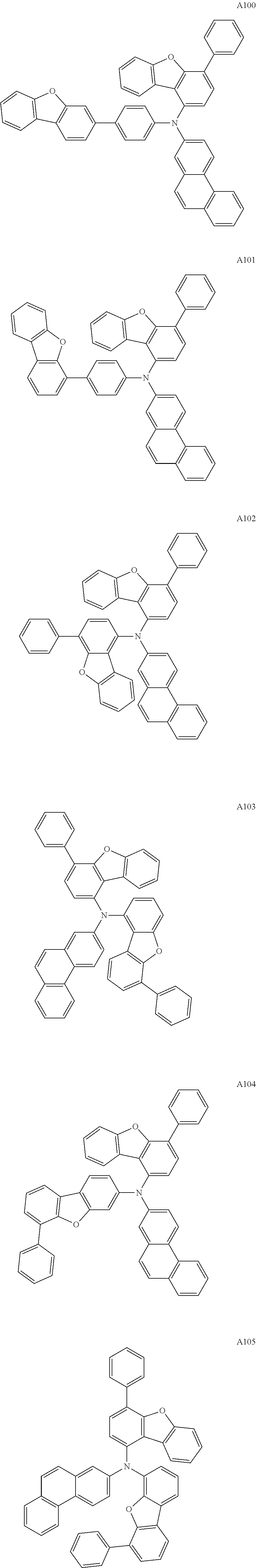

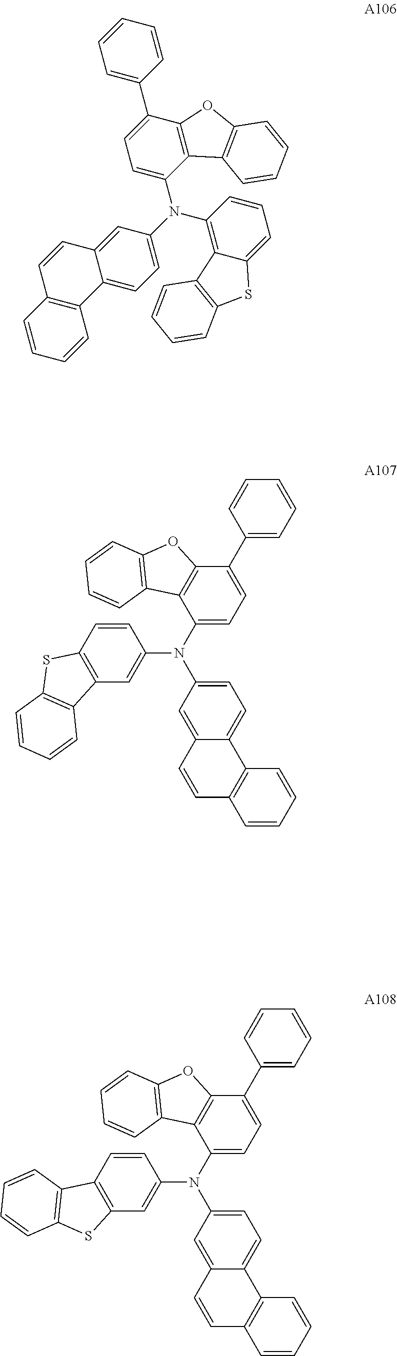 [SMILES: c1ccc(-c2ccc(N(c3ccc4c(ccc5ccccc54)c3)c3cccc4sc5ccccc5c34)c3c2oc2ccccc23)cc1 ; c1ccc(-c2ccc(N(c3ccc4c(ccc5ccccc54)c3)c3ccc4sc5ccccc5c4c3)c3c2oc2ccccc23)cc1 ; c1ccc(-c2ccc(N(c3ccc4c(ccc5ccccc54)c3)c3ccc4c(c3)sc3ccccc34)c3c2oc2ccccc23)cc1]

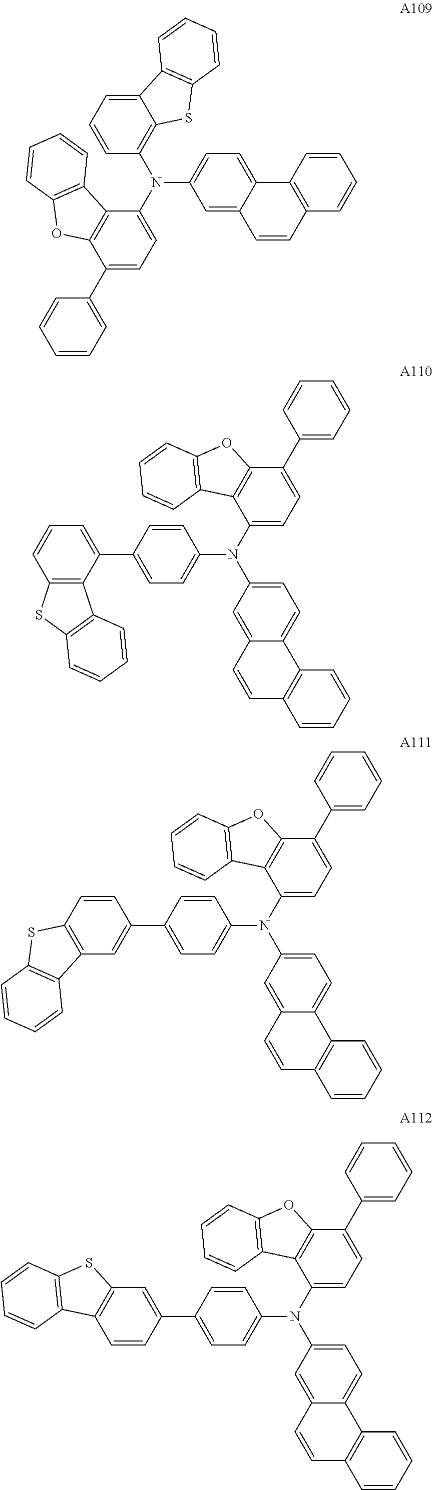

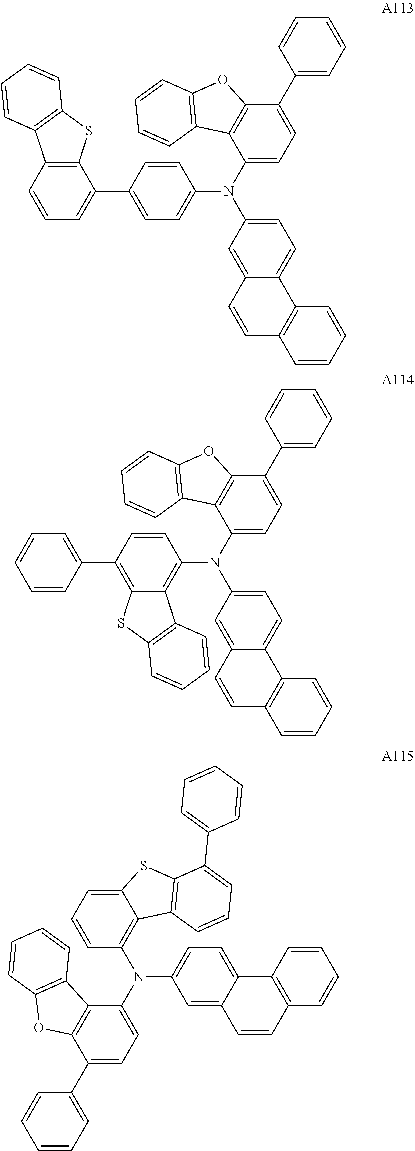

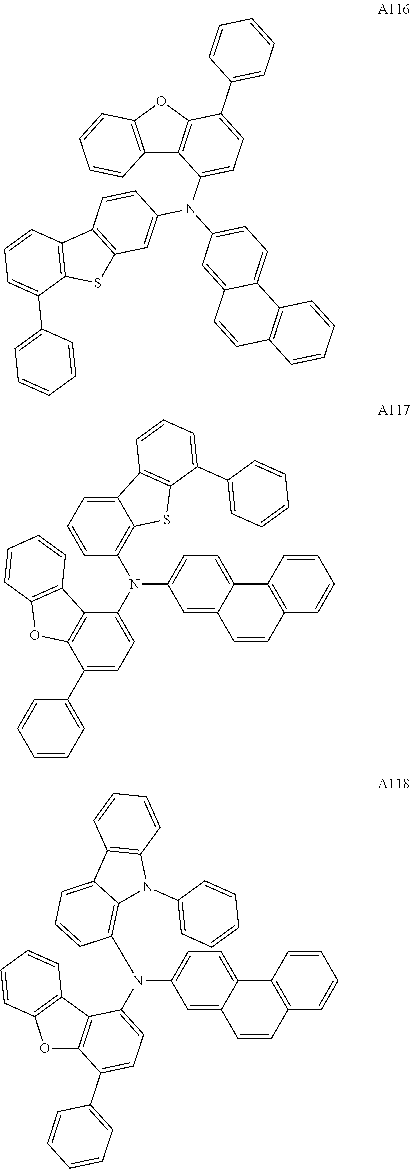 [SMILES: c1ccc(-c2ccc(N(c3ccc4c(ccc5ccccc54)c3)c3ccc4c(c3)sc3c(-c5ccccc5)cccc34)c3c2oc2ccccc23)cc1 ; c1ccc(-c2ccc(N(c3ccc4c(ccc5ccccc54)c3)c3cccc4c3sc3c(-c5ccccc5)cccc34)c3c2oc2ccccc23)cc1 ; c1ccc(-c2ccc(N(c3ccc4c(ccc5ccccc54)c3)c3cccc4c5ccccc5n(-c5ccccc5)c34)c3c2oc2ccccc23)cc1]

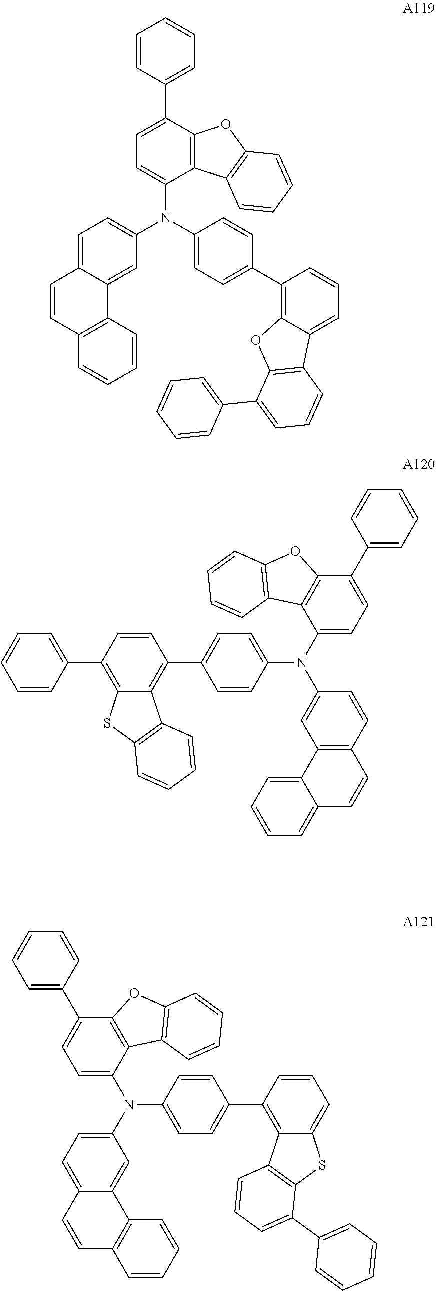 [SMILES: c1ccc(-c2cccc3c2oc2c(-c4ccc(N(c5ccc6ccc7ccccc7c6c5)c5ccc(-c6ccccc6)c6oc7ccccc7c56)cc4)cccc23)cc1 ; c1ccc(-c2ccc(N(c3ccc(-c4ccc(-c5ccccc5)c5sc6ccccc6c45)cc3)c3ccc4ccc5ccccc5c4c3)c3c2oc2ccccc23)cc1 ; c1ccc(-c2ccc(N(c3ccc(-c4cccc5sc6c(-c7ccccc7)cccc6c45)cc3)c3ccc4ccc5ccccc5c4c3)c3c2oc2ccccc23)cc1]

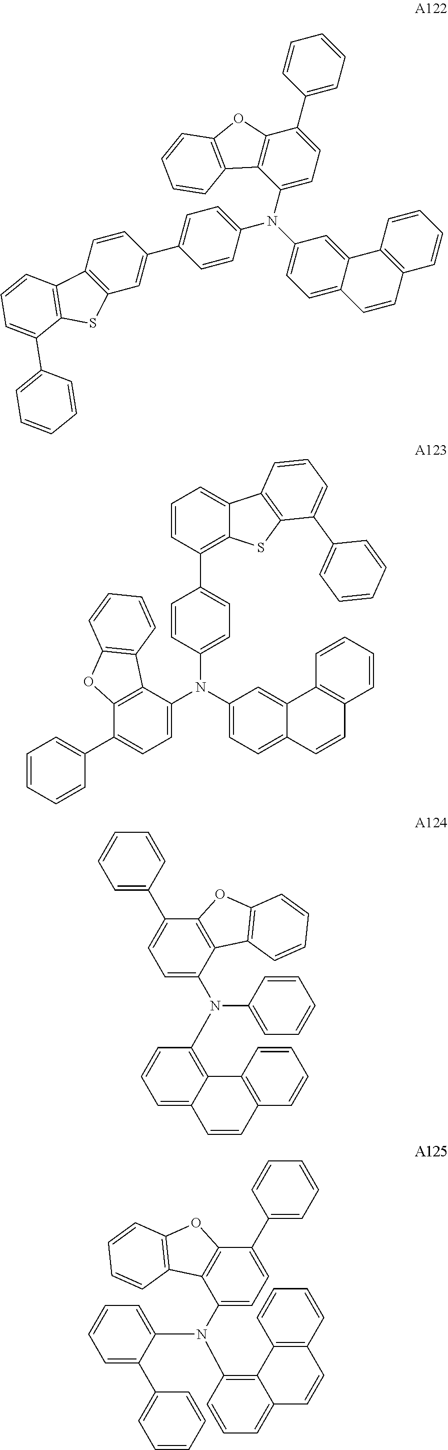

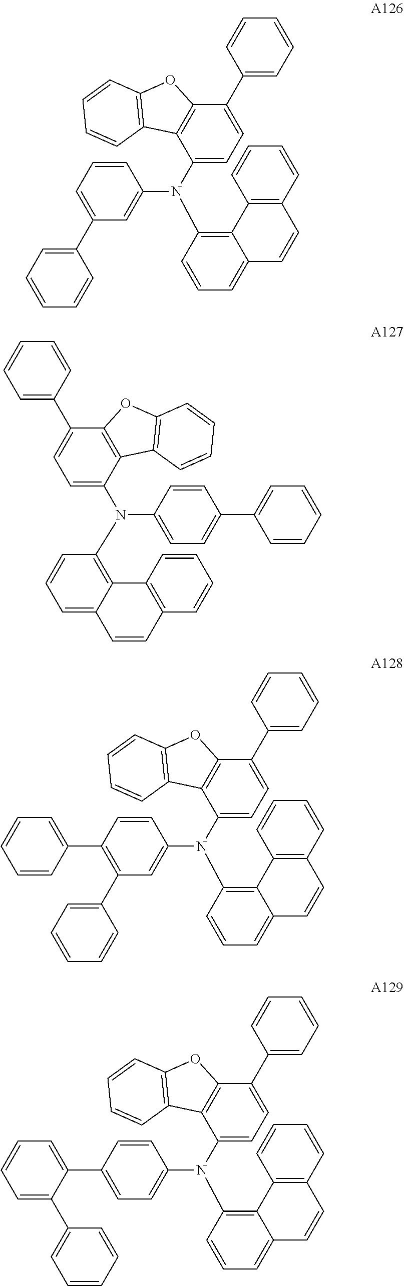

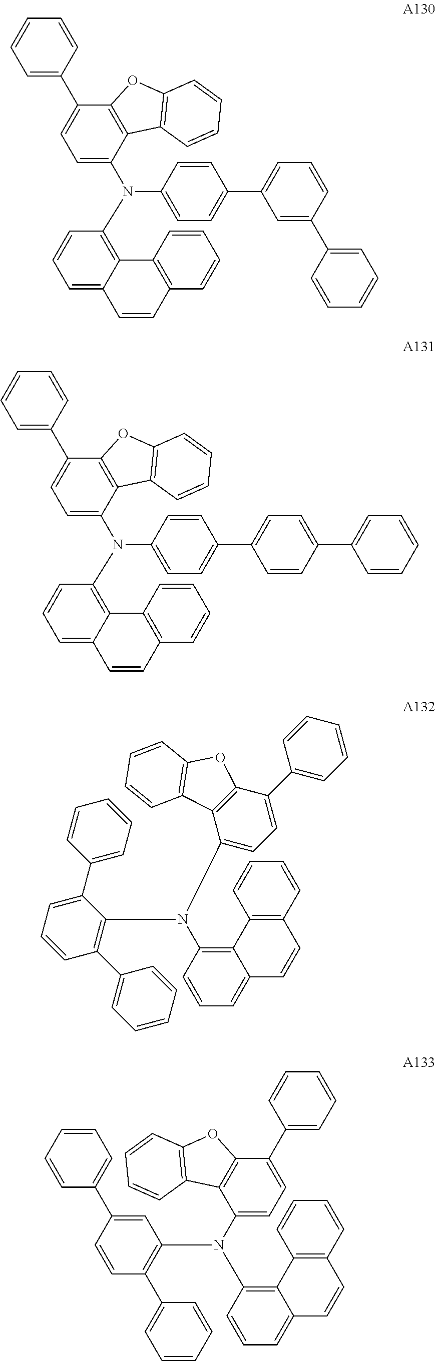 [SMILES: c1ccc(-c2cccc(-c3ccc(N(c4cccc5ccc6ccccc6c45)c4ccc(-c5ccccc5)c5oc6ccccc6c45)cc3)c2)cc1 ; c1ccc(-c2ccc(-c3ccc(N(c4cccc5ccc6ccccc6c45)c4ccc(-c5ccccc5)c5oc6ccccc6c45)cc3)cc2)cc1 ; c1ccc(-c2cccc(-c3ccccc3)c2N(c2cccc3ccc4ccccc4c23)c2ccc(-c3ccccc3)c3oc4ccccc4c23)cc1 ; c1ccc(-c2ccc(-c3ccccc3)c(N(c3cccc4ccc5ccccc5c34)c3ccc(-c4ccccc4)c4oc5ccccc5c34)c2)cc1]

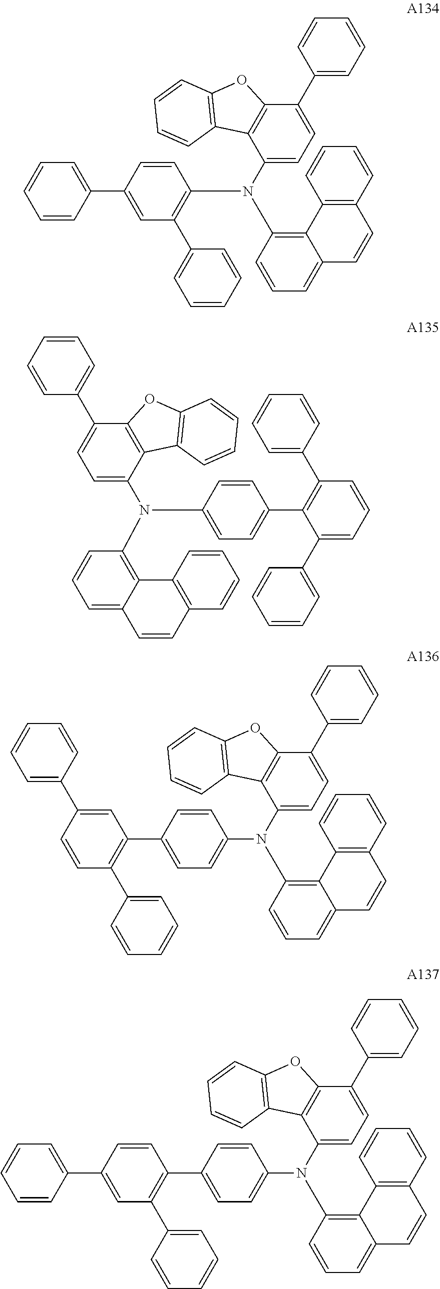 [SMILES: c1ccc(-c2ccc(N(c3cccc4ccc5ccccc5c34)c3ccc(-c4ccccc4)c4oc5ccccc5c34)c(-c3ccccc3)c2)cc1 ; c1ccc(-c2cccc(-c3ccccc3)c2-c2ccc(N(c3cccc4ccc5ccccc5c34)c3ccc(-c4ccccc4)c4oc5ccccc5c34)cc2)cc1 ; c1ccc(-c2ccc(-c3ccccc3)c(-c3ccc(N(c4cccc5ccc6ccccc6c45)c4ccc(-c5ccccc5)c5oc6ccccc6c45)cc3)c2)cc1 ; c1ccc(-c2ccc(-c3ccc(N(c4cccc5ccc6ccccc6c45)c4ccc(-c5ccccc5)c5oc6ccccc6c45)cc3)c(-c3ccccc3)c2)cc1]

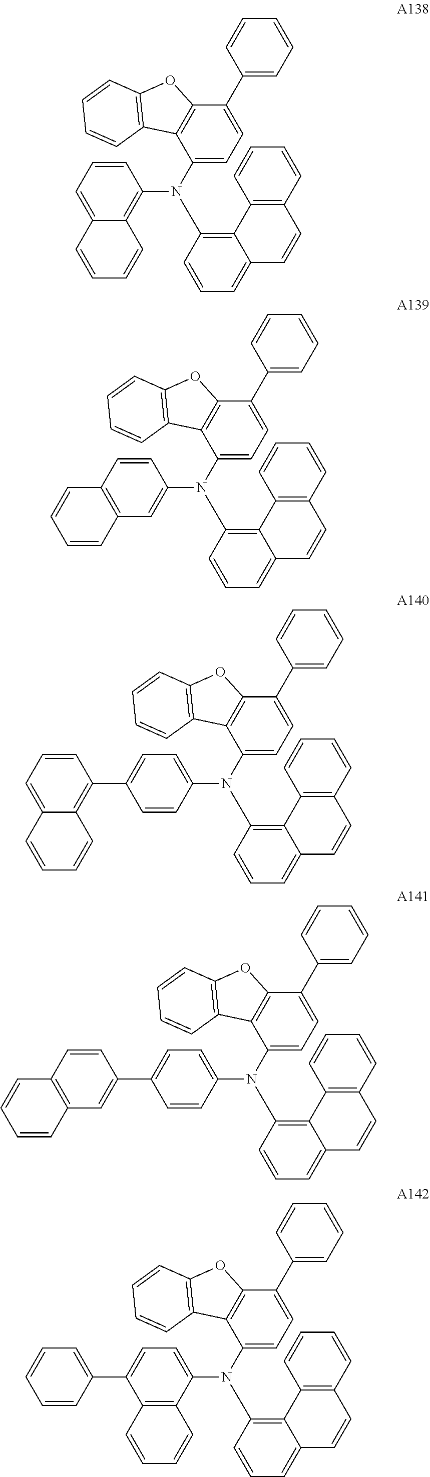

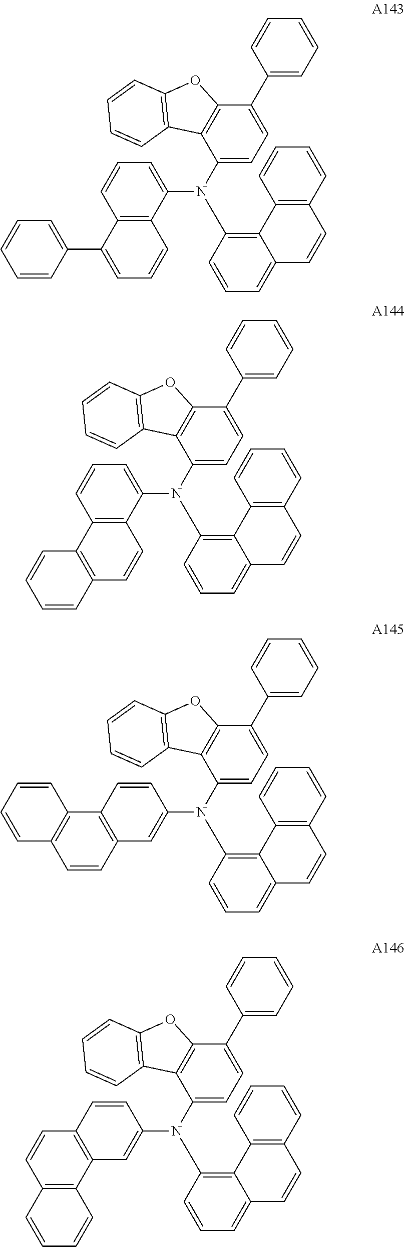

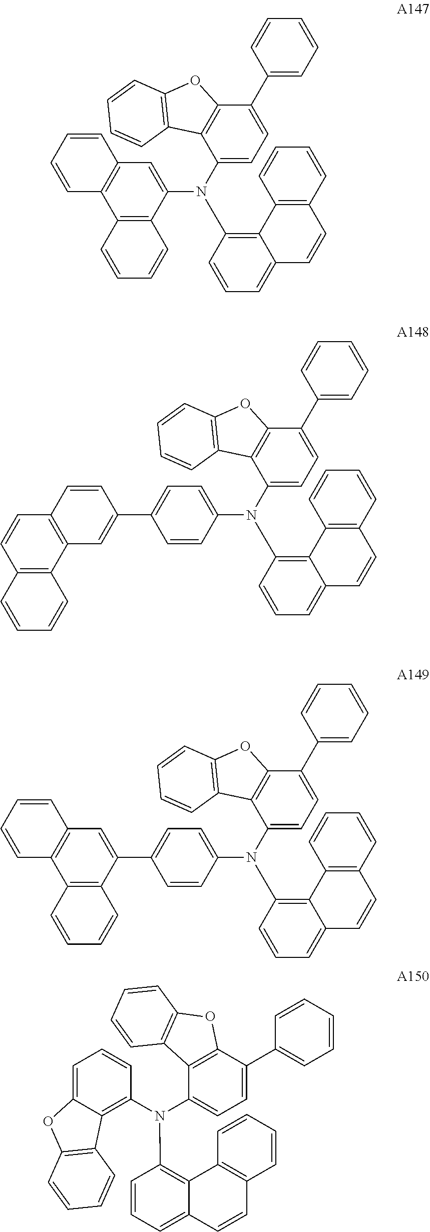

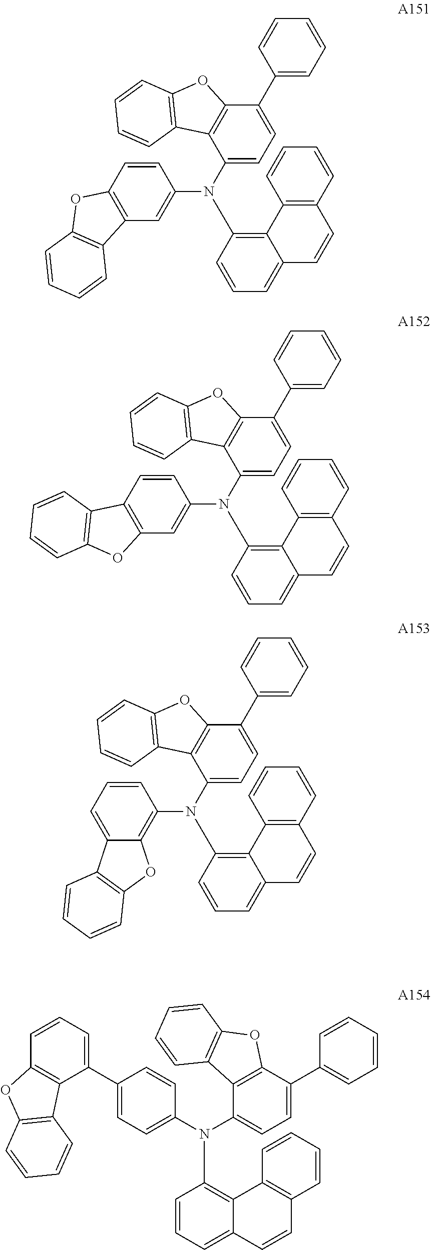

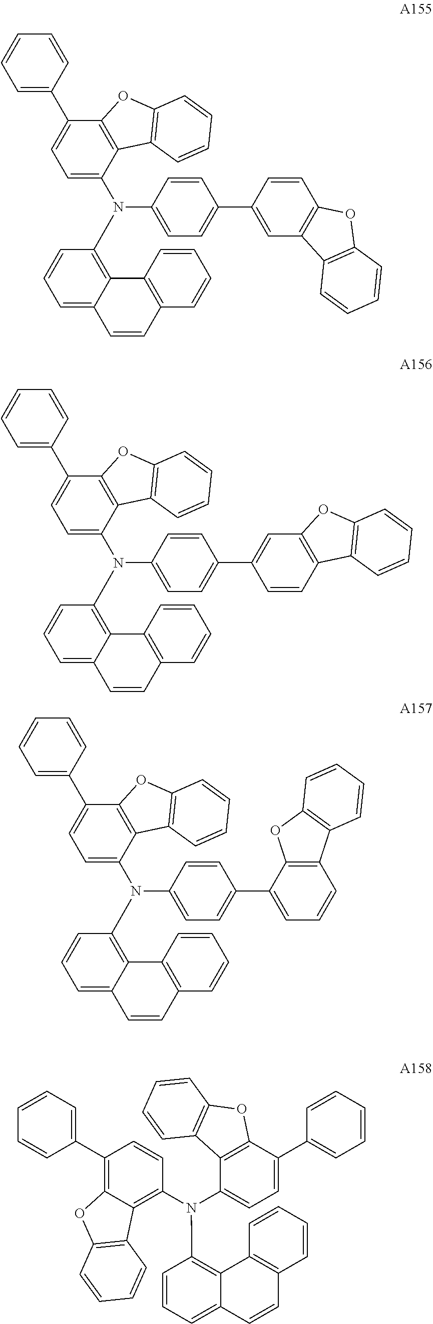 [SMILES: c1ccc(-c2ccc(N(c3ccc(-c4ccc5oc6ccccc6c5c4)cc3)c3cccc4ccc5ccccc5c34)c3c2oc2ccccc23)cc1 ; c1ccc(-c2ccc(N(c3ccc(-c4ccc5c(c4)oc4ccccc45)cc3)c3cccc4ccc5ccccc5c34)c3c2oc2ccccc23)cc1 ; c1ccc(-c2ccc(N(c3ccc(-c4cccc5c4oc4ccccc45)cc3)c3cccc4ccc5ccccc5c34)c3c2oc2ccccc23)cc1 ; c1ccc(-c2ccc(N(c3cccc4ccc5ccccc5c34)c3ccc(-c4ccccc4)c4oc5ccccc5c34)c3c2oc2ccccc23)cc1]

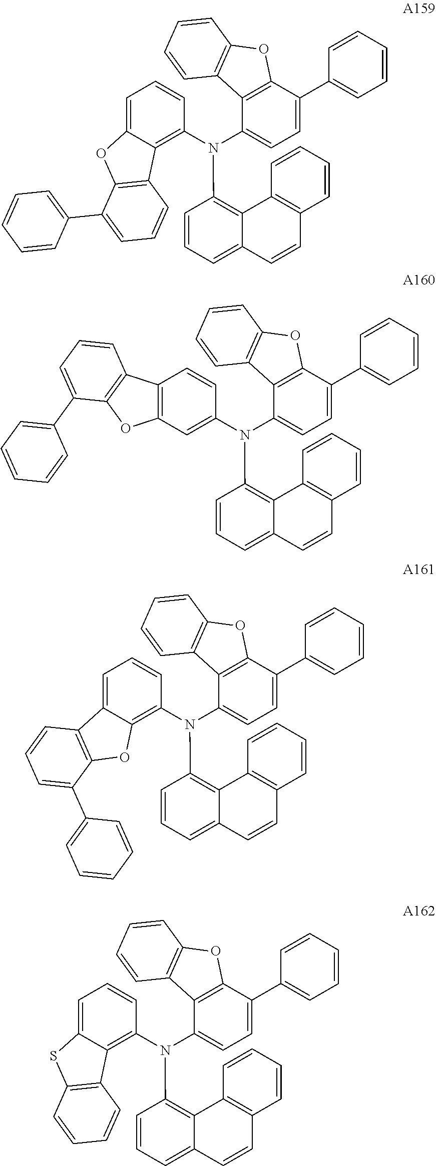

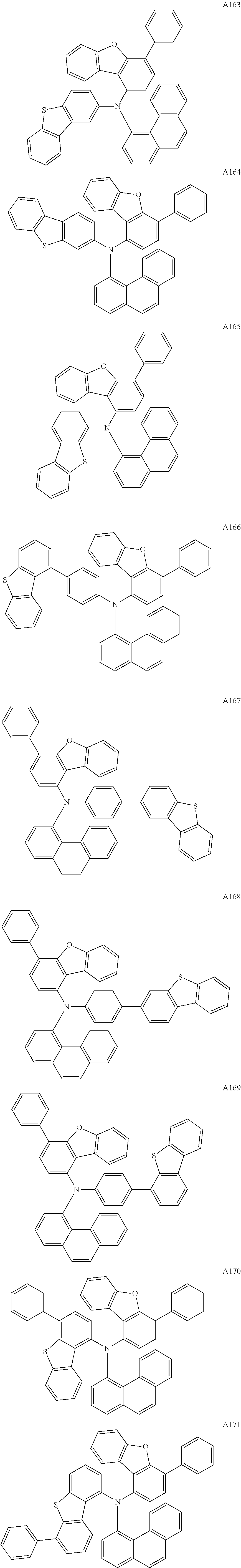

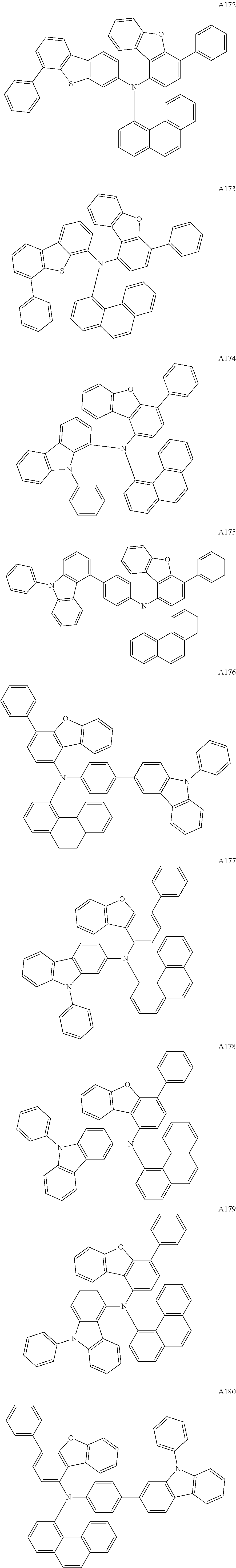

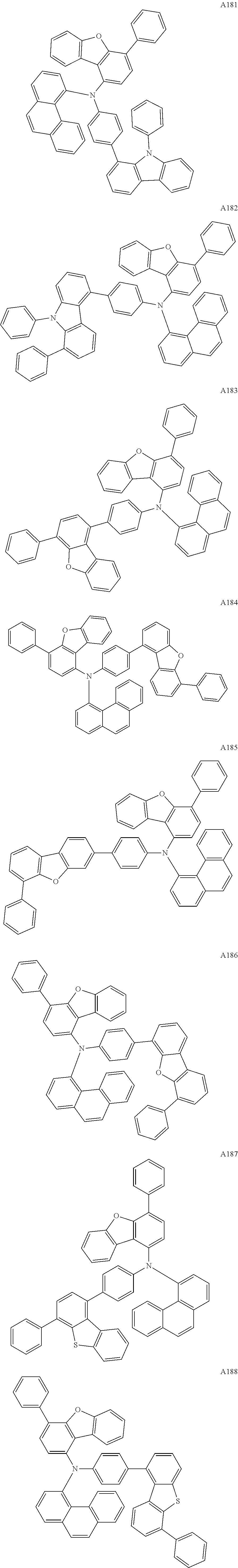

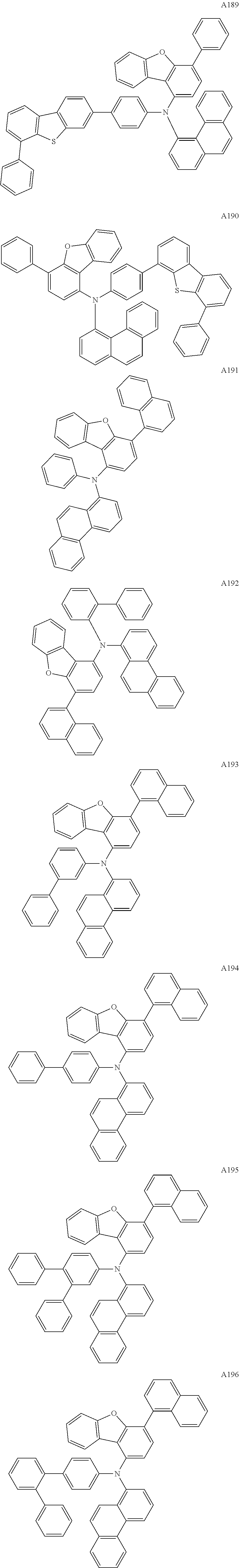

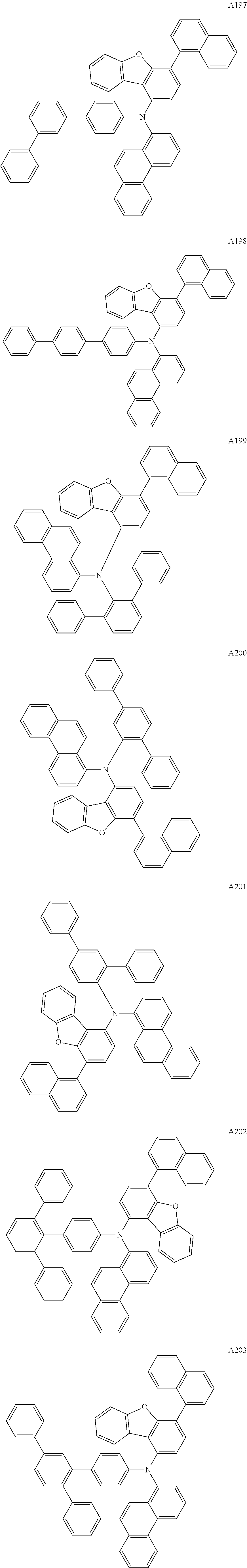

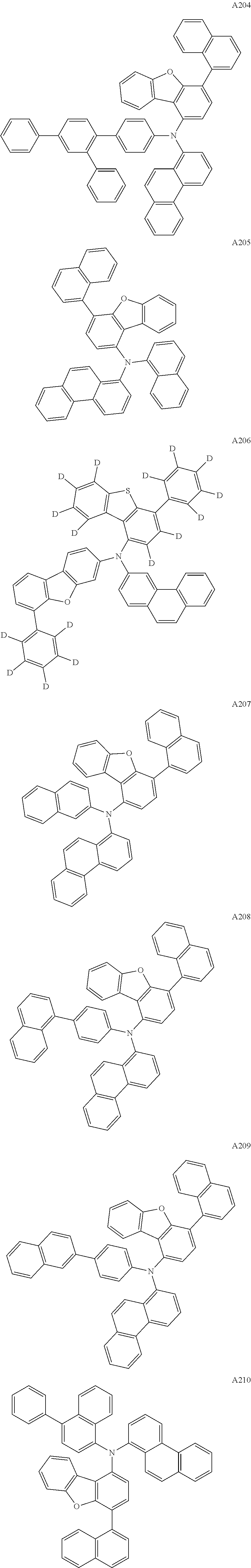

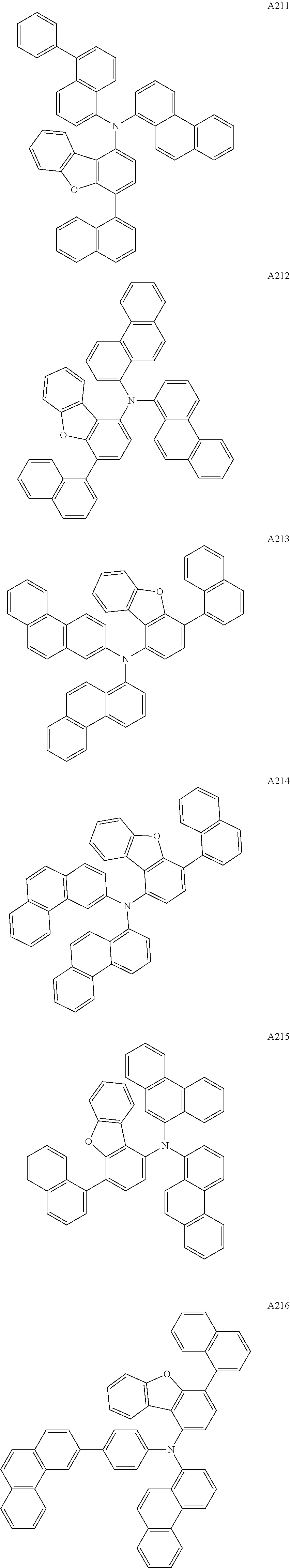

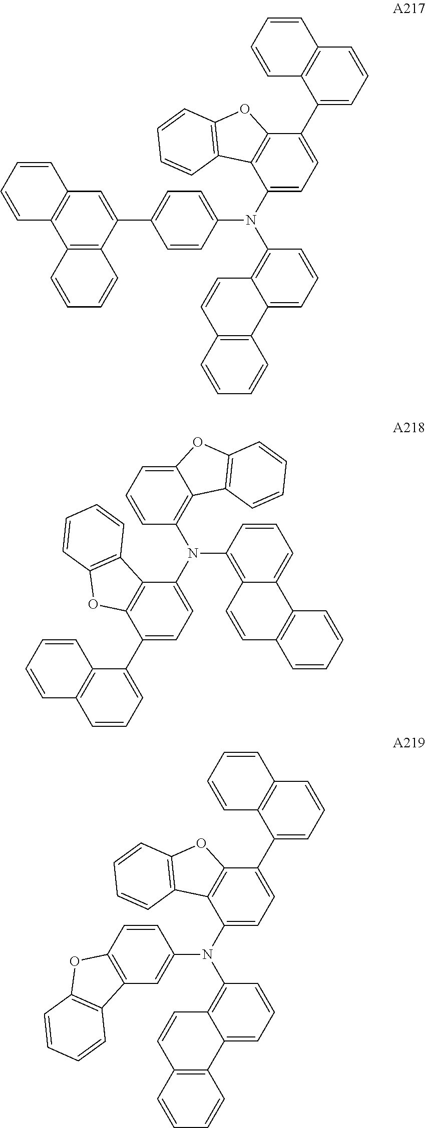 [SMILES: c1ccc2c(-c3ccc(N(c4ccc(-c5cc6ccccc6c6ccccc56)cc4)c4cccc5c4ccc4ccccc45)c4c3oc3ccccc34)cccc2c1 ; c1ccc2c(-c3ccc(N(c4cccc5c4ccc4ccccc45)c4cccc5oc6ccccc6c45)c4c3oc3ccccc34)cccc2c1 ; c1ccc2c(-c3ccc(N(c4ccc5oc6ccccc6c5c4)c4cccc5c4ccc4ccccc45)c4c3oc3ccccc34)cccc2c1]

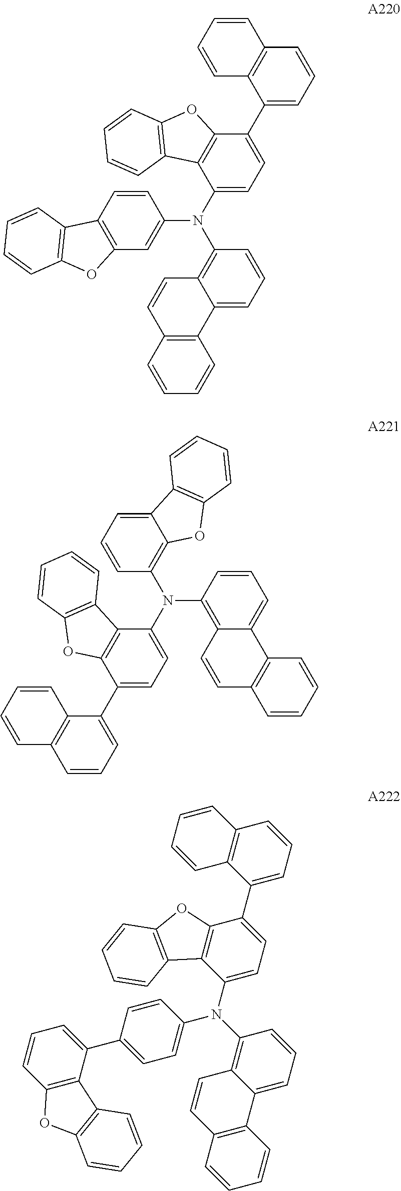

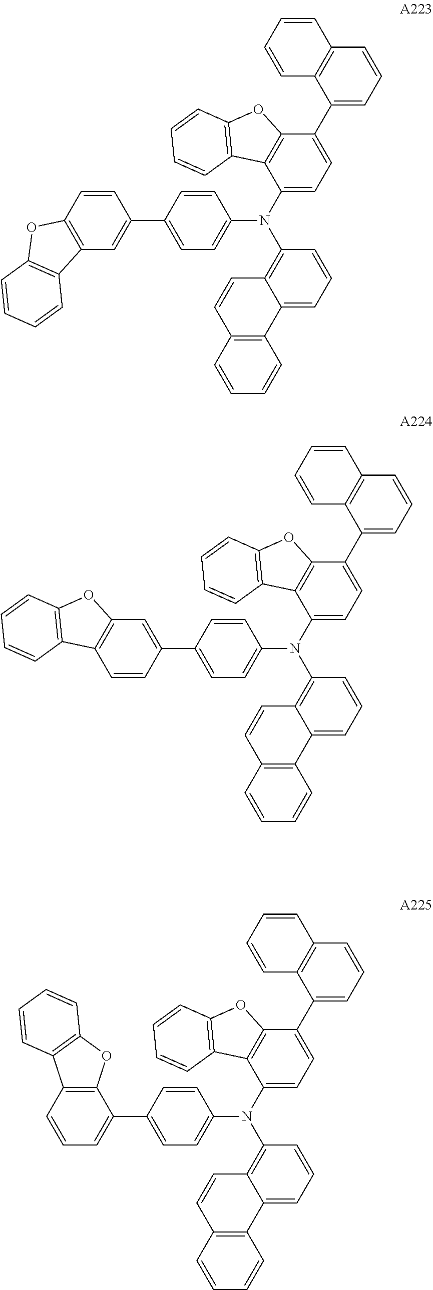 [SMILES: c1ccc2c(-c3ccc(N(c4ccc(-c5ccc6oc7ccccc7c6c5)cc4)c4cccc5c4ccc4ccccc45)c4c3oc3ccccc34)cccc2c1 ; c1ccc2c(-c3ccc(N(c4ccc(-c5ccc6c(c5)oc5ccccc56)cc4)c4cccc5c4ccc4ccccc45)c4c3oc3ccccc34)cccc2c1 ; c1ccc2c(-c3ccc(N(c4ccc(-c5cccc6c5oc5ccccc56)cc4)c4cccc5c4ccc4ccccc45)c4c3oc3ccccc34)cccc2c1]

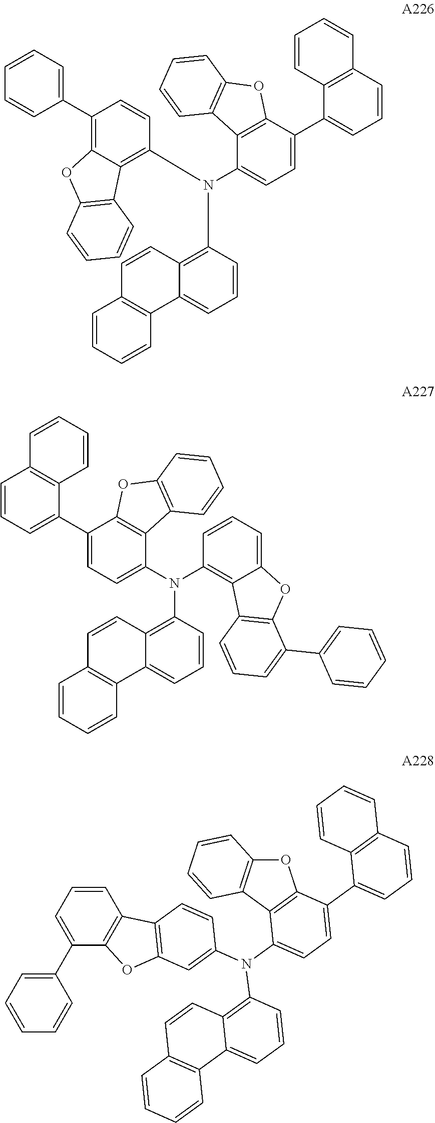 [SMILES: c1ccc(-c2ccc(N(c3cccc4c3ccc3ccccc34)c3ccc(-c4cccc5ccccc45)c4oc5ccccc5c34)c3c2oc2ccccc23)cc1 ; c1ccc(-c2cccc3c2oc2cccc(N(c4cccc5c4ccc4ccccc45)c4ccc(-c5cccc6ccccc56)c5oc6ccccc6c45)c23)cc1 ; c1ccc(-c2cccc3c2oc2cc(N(c4cccc5c4ccc4ccccc45)c4ccc(-c5cccc6ccccc56)c5oc6ccccc6c45)ccc23)cc1]

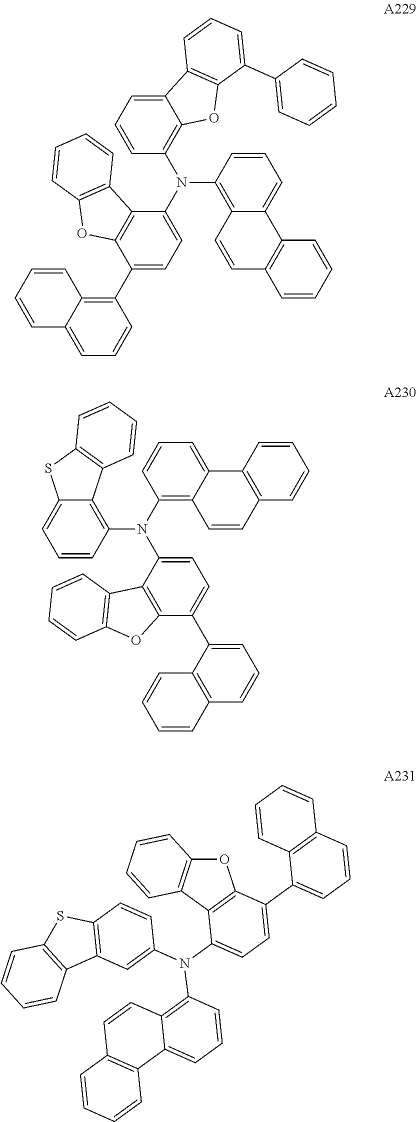

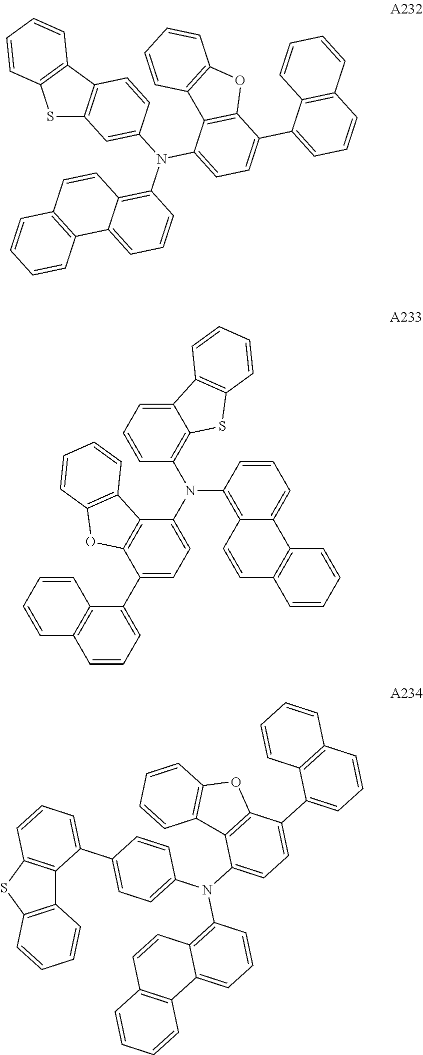 [SMILES: c1ccc2c(-c3ccc(N(c4ccc5c(c4)sc4ccccc45)c4cccc5c4ccc4ccccc45)c4c3oc3ccccc34)cccc2c1 ; c1ccc2c(-c3ccc(N(c4cccc5c4ccc4ccccc45)c4cccc5c4sc4ccccc45)c4c3oc3ccccc34)cccc2c1 ; c1ccc2c(-c3ccc(N(c4ccc(-c5cccc6sc7ccccc7c56)cc4)c4cccc5c4ccc4ccccc45)c4c3oc3ccccc34)cccc2c1]

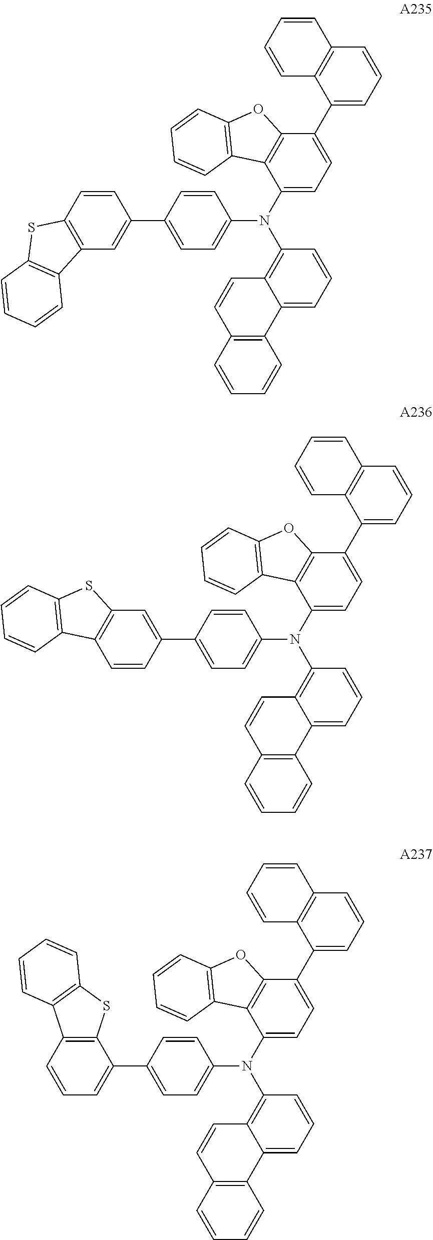 [SMILES: c1ccc2c(-c3ccc(N(c4ccc(-c5ccc6sc7ccccc7c6c5)cc4)c4cccc5c4ccc4ccccc45)c4c3oc3ccccc34)cccc2c1 ; c1ccc2c(-c3ccc(N(c4ccc(-c5ccc6c(c5)sc5ccccc56)cc4)c4cccc5c4ccc4ccccc45)c4c3oc3ccccc34)cccc2c1 ; c1ccc2c(-c3ccc(N(c4ccc(-c5cccc6c5sc5ccccc56)cc4)c4cccc5c4ccc4ccccc45)c4c3oc3ccccc34)cccc2c1]

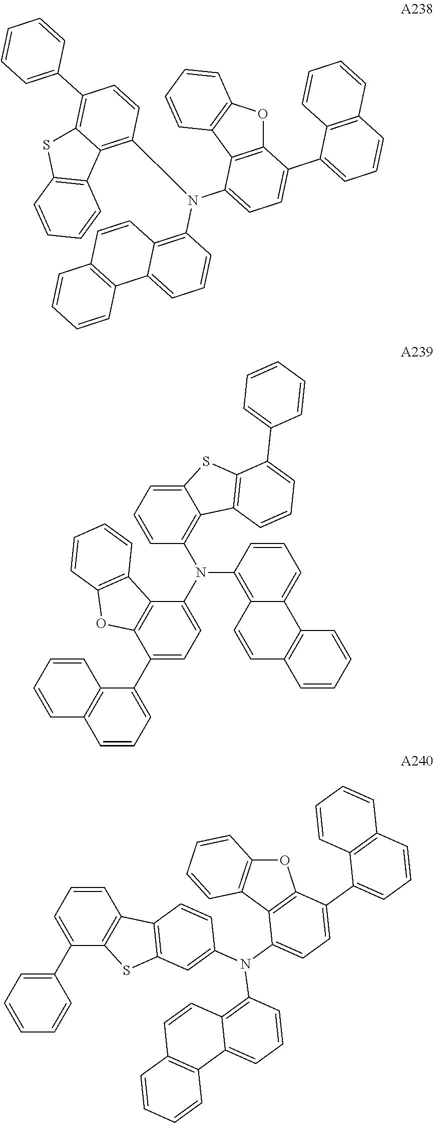 [SMILES: c1ccc(-c2ccc(N(c3cccc4c3ccc3ccccc34)c3ccc(-c4cccc5ccccc45)c4oc5ccccc5c34)c3c2sc2ccccc23)cc1 ; c1ccc(-c2cccc3c2sc2cccc(N(c4cccc5c4ccc4ccccc45)c4ccc(-c5cccc6ccccc56)c5oc6ccccc6c45)c23)cc1 ; c1ccc(-c2cccc3c2sc2cc(N(c4cccc5c4ccc4ccccc45)c4ccc(-c5cccc6ccccc56)c5oc6ccccc6c45)ccc23)cc1]

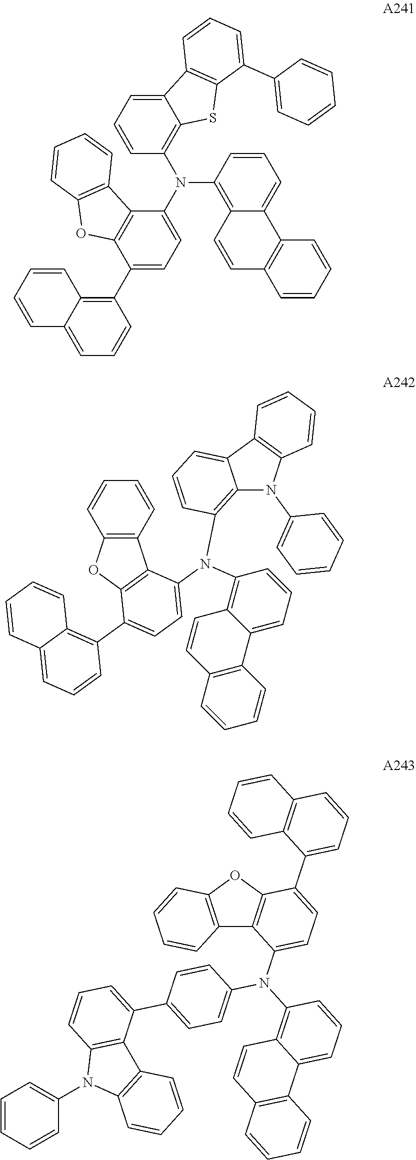 [SMILES: c1ccc(-c2cccc3c2sc2c(N(c4cccc5c4ccc4ccccc45)c4ccc(-c5cccc6ccccc56)c5oc6ccccc6c45)cccc23)cc1 ; c1ccc(-n2c3ccccc3c3cccc(N(c4cccc5c4ccc4ccccc45)c4ccc(-c5cccc6ccccc56)c5oc6ccccc6c45)c32)cc1 ; c1ccc(-n2c3ccccc3c3c(-c4ccc(N(c5cccc6c5ccc5ccccc56)c5ccc(-c6cccc7ccccc67)c6oc7ccccc7c56)cc4)cccc32)cc1]

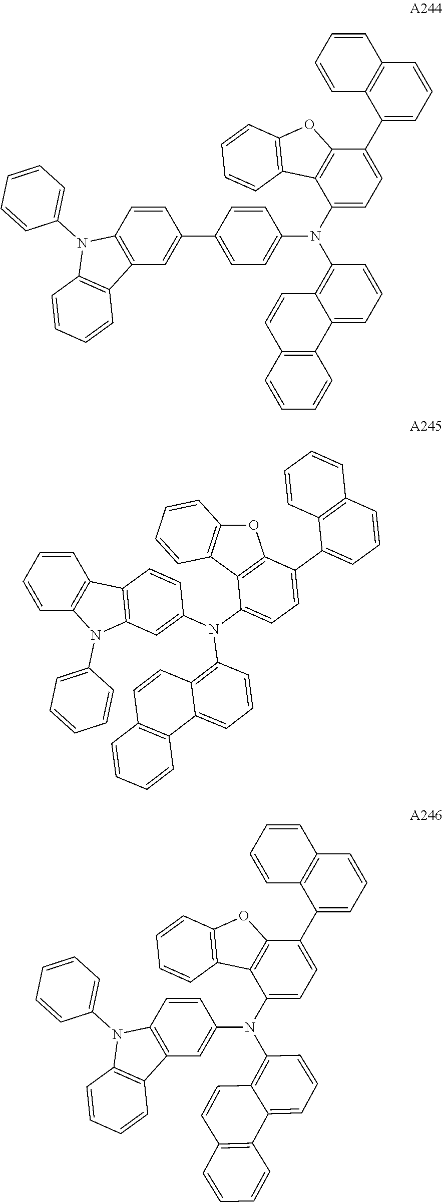

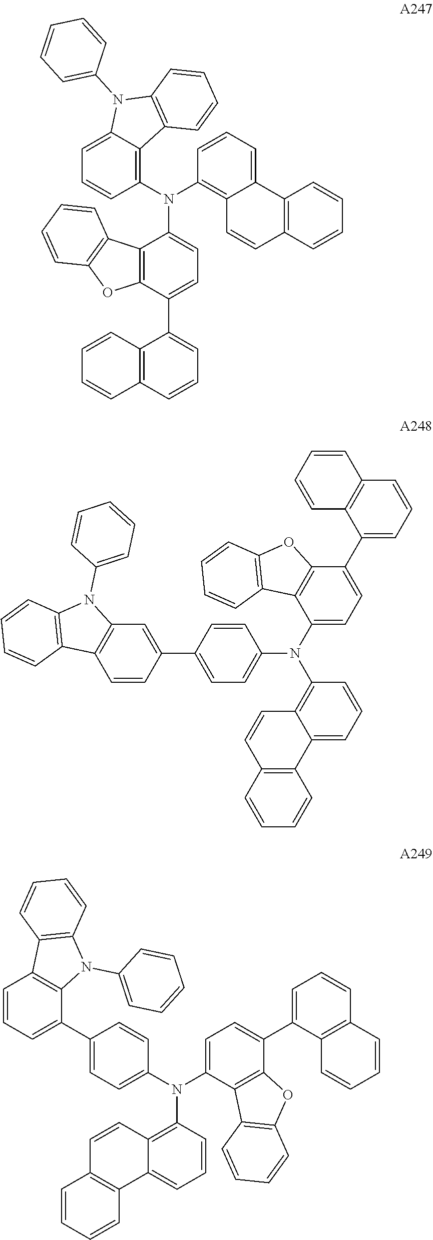 [SMILES: c1ccc(-n2c3ccccc3c3c(N(c4cccc5c4ccc4ccccc45)c4ccc(-c5cccc6ccccc56)c5oc6ccccc6c45)cccc32)cc1 ; c1ccc(-n2c3ccccc3c3ccc(-c4ccc(N(c5cccc6c5ccc5ccccc56)c5ccc(-c6cccc7ccccc67)c6oc7ccccc7c56)cc4)cc32)cc1 ; c1ccc(-n2c3ccccc3c3cccc(-c4ccc(N(c5cccc6c5ccc5ccccc56)c5ccc(-c6cccc7ccccc67)c6oc7ccccc7c56)cc4)c32)cc1]

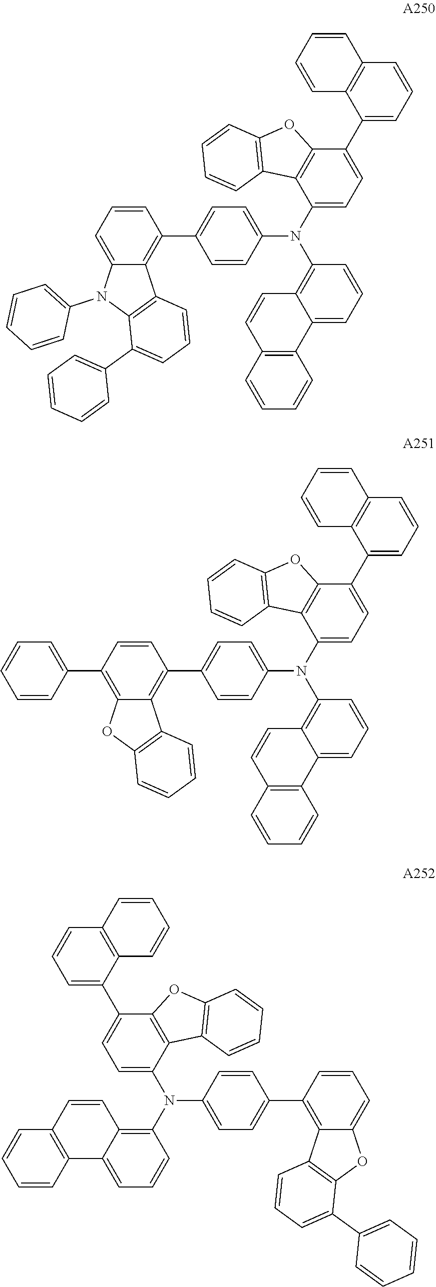 [SMILES: c1ccc(-c2cccc3c4c(-c5ccc(N(c6cccc7c6ccc6ccccc67)c6ccc(-c7cccc8ccccc78)c7oc8ccccc8c67)cc5)cccc4n(-c4ccccc4)c23)cc1 ; c1ccc(-c2ccc(-c3ccc(N(c4cccc5c4ccc4ccccc45)c4ccc(-c5cccc6ccccc56)c5oc6ccccc6c45)cc3)c3c2oc2ccccc23)cc1 ; c1ccc(-c2cccc3c2oc2cccc(-c4ccc(N(c5cccc6c5ccc5ccccc56)c5ccc(-c6cccc7ccccc67)c6oc7ccccc7c56)cc4)c23)cc1]

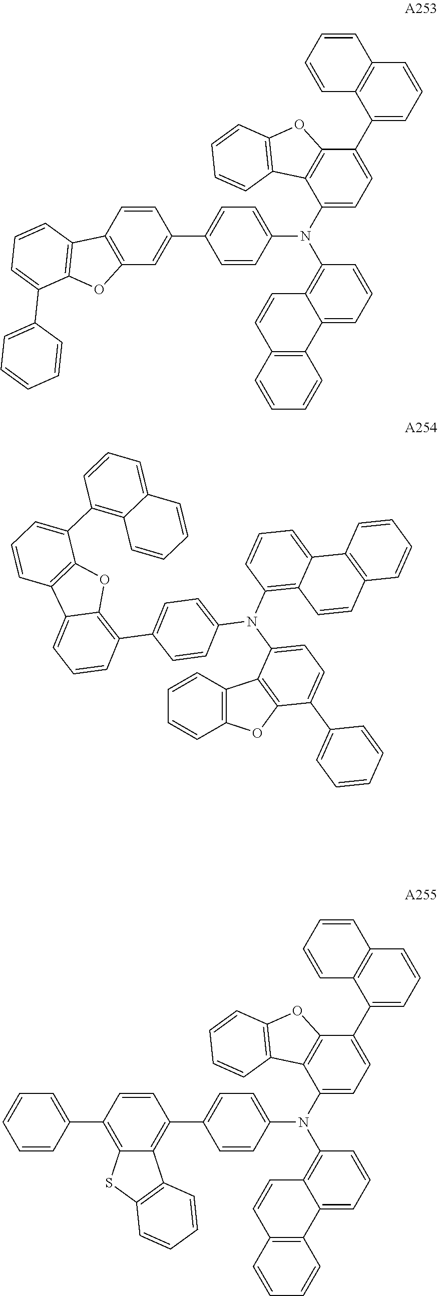 [SMILES: c1ccc(-c2cccc3c2oc2cc(-c4ccc(N(c5cccc6c5ccc5ccccc56)c5ccc(-c6cccc7ccccc67)c6oc7ccccc7c56)cc4)ccc23)cc1 ; c1ccc(-c2ccc(N(c3ccc(-c4cccc5c4oc4c(-c6cccc7ccccc67)cccc45)cc3)c3cccc4c3ccc3ccccc34)c3c2oc2ccccc23)cc1 ; c1ccc(-c2ccc(-c3ccc(N(c4cccc5c4ccc4ccccc45)c4ccc(-c5cccc6ccccc56)c5oc6ccccc6c45)cc3)c3c2sc2ccccc23)cc1]

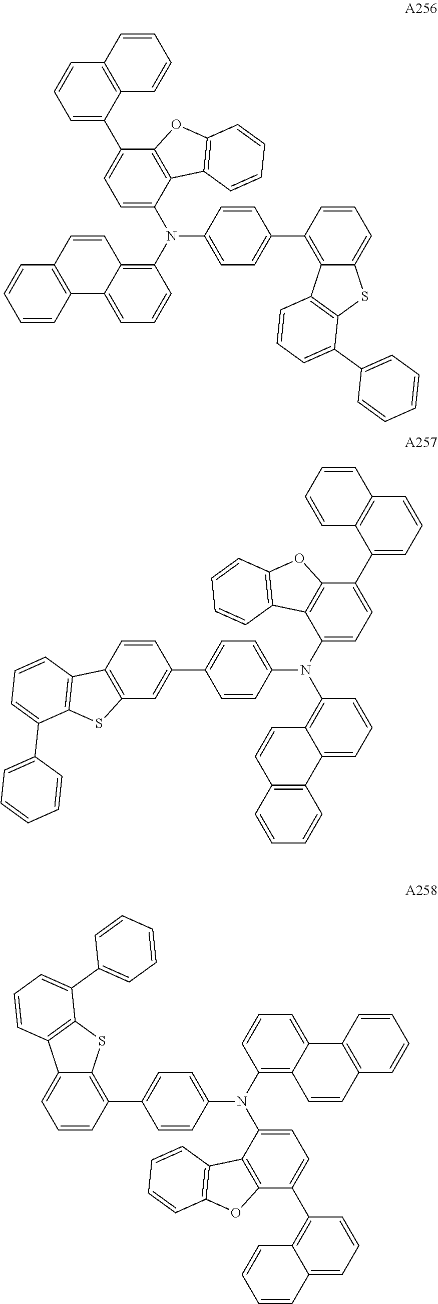 [SMILES: c1ccc(-c2cccc3c2sc2cccc(-c4ccc(N(c5cccc6c5ccc5ccccc56)c5ccc(-c6cccc7ccccc67)c6oc7ccccc7c56)cc4)c23)cc1 ; c1ccc(-c2cccc3c2sc2cc(-c4ccc(N(c5cccc6c5ccc5ccccc56)c5ccc(-c6cccc7ccccc67)c6oc7ccccc7c56)cc4)ccc23)cc1 ; c1ccc(-c2cccc3c2sc2c(-c4ccc(N(c5cccc6c5ccc5ccccc56)c5ccc(-c6cccc7ccccc67)c6oc7ccccc7c56)cc4)cccc23)cc1]

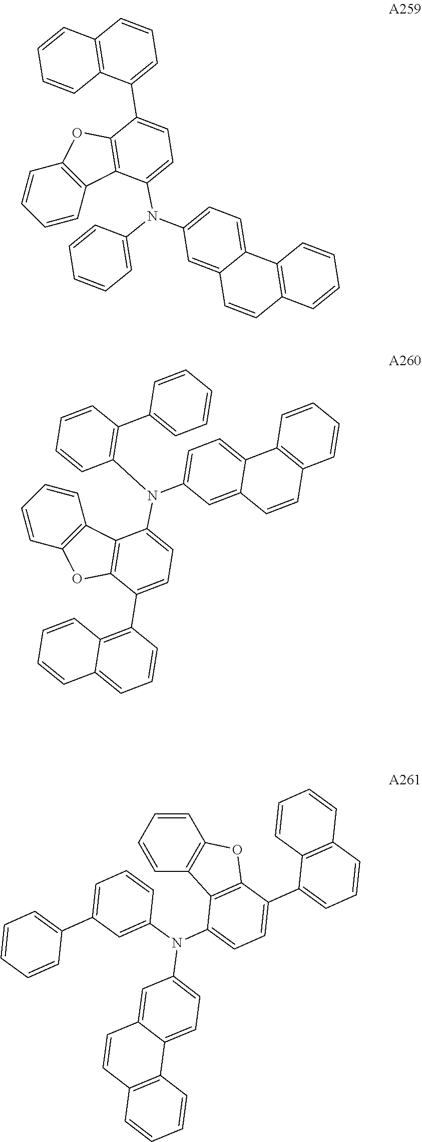 [SMILES: c1ccc(N(c2ccc3c(ccc4ccccc43)c2)c2ccc(-c3cccc4ccccc34)c3oc4ccccc4c23)cc1 ; c1ccc(-c2ccccc2N(c2ccc3c(ccc4ccccc43)c2)c2ccc(-c3cccc4ccccc34)c3oc4ccccc4c23)cc1 ; c1ccc(-c2cccc(N(c3ccc4c(ccc5ccccc54)c3)c3ccc(-c4cccc5ccccc45)c4oc5ccccc5c34)c2)cc1]

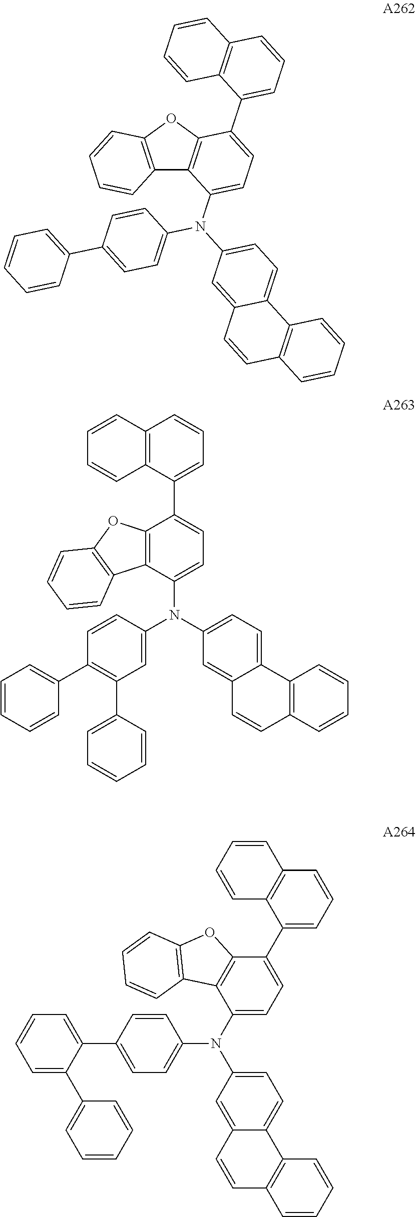 [SMILES: c1ccc(-c2ccc(N(c3ccc4c(ccc5ccccc54)c3)c3ccc(-c4cccc5ccccc45)c4oc5ccccc5c34)cc2)cc1 ; c1ccc(-c2ccc(N(c3ccc4c(ccc5ccccc54)c3)c3ccc(-c4cccc5ccccc45)c4oc5ccccc5c34)cc2-c2ccccc2)cc1 ; c1ccc(-c2ccccc2-c2ccc(N(c3ccc4c(ccc5ccccc54)c3)c3ccc(-c4cccc5ccccc45)c4oc5ccccc5c34)cc2)cc1]

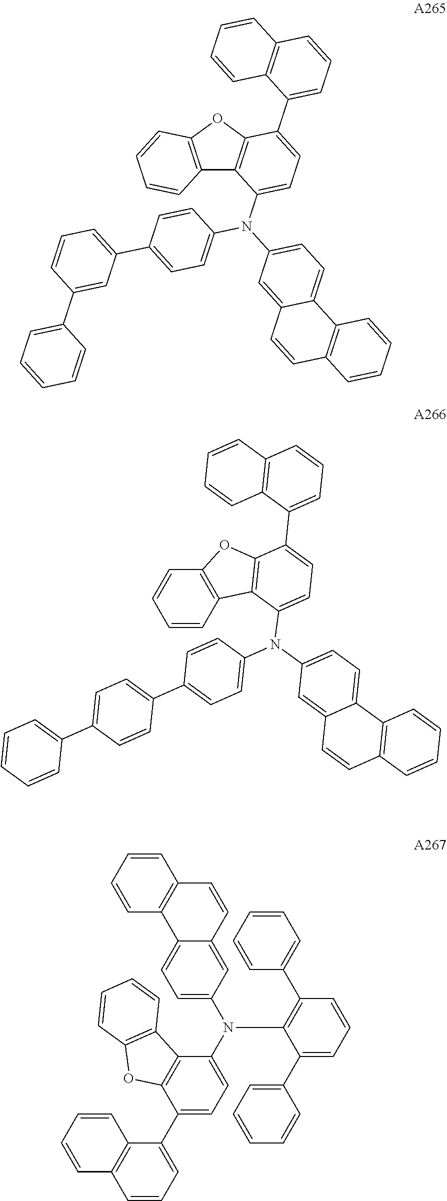 [SMILES: c1ccc(-c2cccc(-c3ccc(N(c4ccc5c(ccc6ccccc65)c4)c4ccc(-c5cccc6ccccc56)c5oc6ccccc6c45)cc3)c2)cc1 ; c1ccc(-c2ccc(-c3ccc(N(c4ccc5c(ccc6ccccc65)c4)c4ccc(-c5cccc6ccccc56)c5oc6ccccc6c45)cc3)cc2)cc1 ; c1ccc(-c2cccc(-c3ccccc3)c2N(c2ccc3c(ccc4ccccc43)c2)c2ccc(-c3cccc4ccccc34)c3oc4ccccc4c23)cc1]

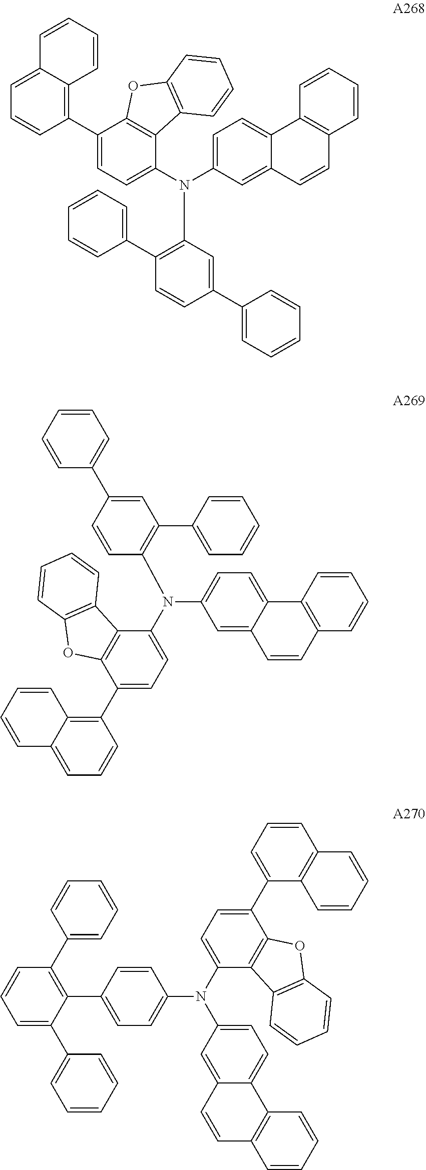 [SMILES: c1ccc(-c2ccc(-c3ccccc3)c(N(c3ccc4c(ccc5ccccc54)c3)c3ccc(-c4cccc5ccccc45)c4oc5ccccc5c34)c2)cc1 ; c1ccc(-c2ccc(N(c3ccc4c(ccc5ccccc54)c3)c3ccc(-c4cccc5ccccc45)c4oc5ccccc5c34)c(-c3ccccc3)c2)cc1 ; c1ccc(-c2cccc(-c3ccccc3)c2-c2ccc(N(c3ccc4c(ccc5ccccc54)c3)c3ccc(-c4cccc5ccccc45)c4oc5ccccc5c34)cc2)cc1]

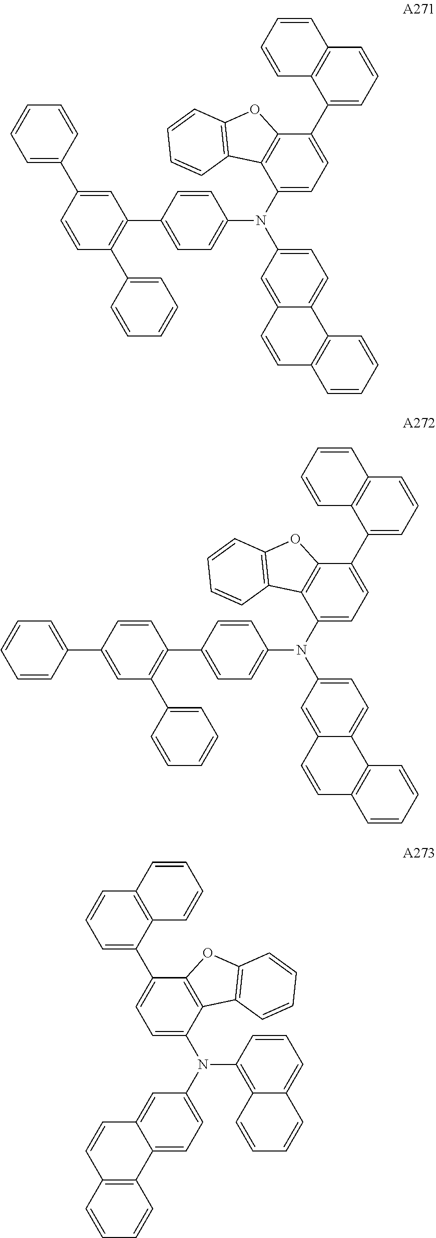 [SMILES: c1ccc(-c2ccc(-c3ccccc3)c(-c3ccc(N(c4ccc5c(ccc6ccccc65)c4)c4ccc(-c5cccc6ccccc56)c5oc6ccccc6c45)cc3)c2)cc1 ; c1ccc(-c2ccc(-c3ccc(N(c4ccc5c(ccc6ccccc65)c4)c4ccc(-c5cccc6ccccc56)c5oc6ccccc6c45)cc3)c(-c3ccccc3)c2)cc1 ; c1ccc2c(-c3ccc(N(c4ccc5c(ccc6ccccc65)c4)c4cccc5ccccc45)c4c3oc3ccccc34)cccc2c1]

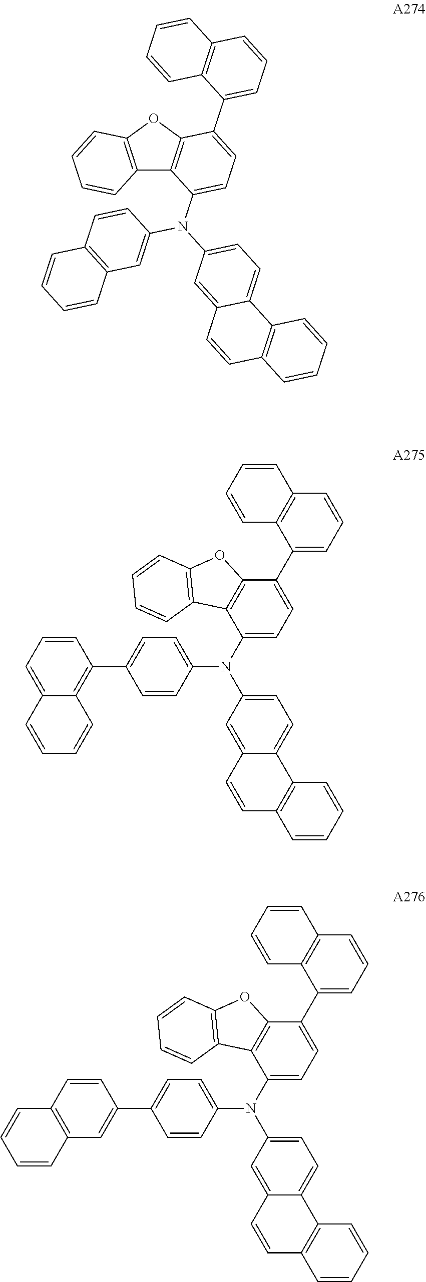 [SMILES: c1ccc2cc(N(c3ccc4c(ccc5ccccc54)c3)c3ccc(-c4cccc5ccccc45)c4oc5ccccc5c34)ccc2c1 ; c1ccc2c(-c3ccc(N(c4ccc5c(ccc6ccccc65)c4)c4ccc(-c5cccc6ccccc56)c5oc6ccccc6c45)cc3)cccc2c1 ; c1ccc2cc(-c3ccc(N(c4ccc5c(ccc6ccccc65)c4)c4ccc(-c5cccc6ccccc56)c5oc6ccccc6c45)cc3)ccc2c1]

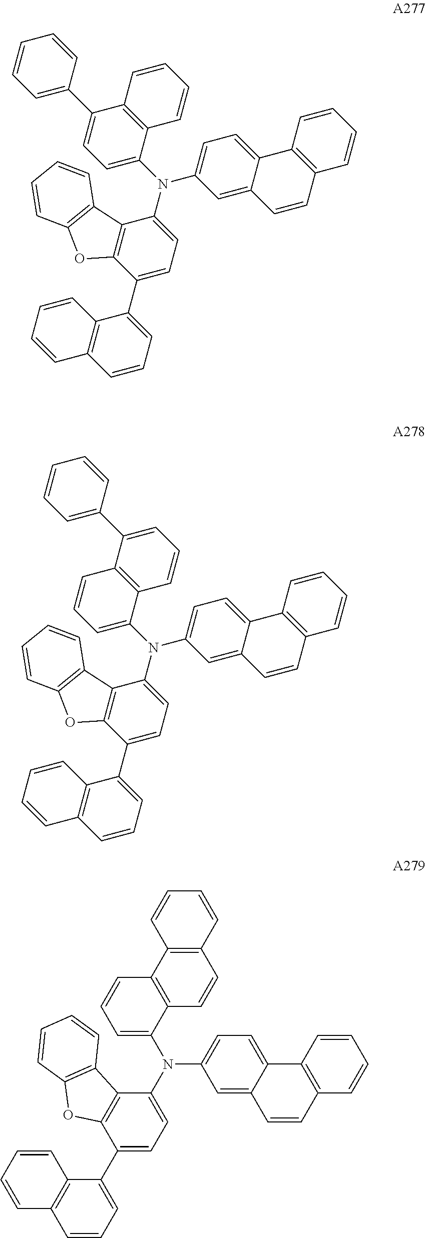 [SMILES: c1ccc(-c2ccc(N(c3ccc4c(ccc5ccccc54)c3)c3ccc(-c4cccc5ccccc45)c4oc5ccccc5c34)c3ccccc23)cc1 ; c1ccc(-c2cccc3c(N(c4ccc5c(ccc6ccccc65)c4)c4ccc(-c5cccc6ccccc56)c5oc6ccccc6c45)cccc23)cc1 ; c1ccc2c(-c3ccc(N(c4ccc5c(ccc6ccccc65)c4)c4cccc5c4ccc4ccccc45)c4c3oc3ccccc34)cccc2c1]

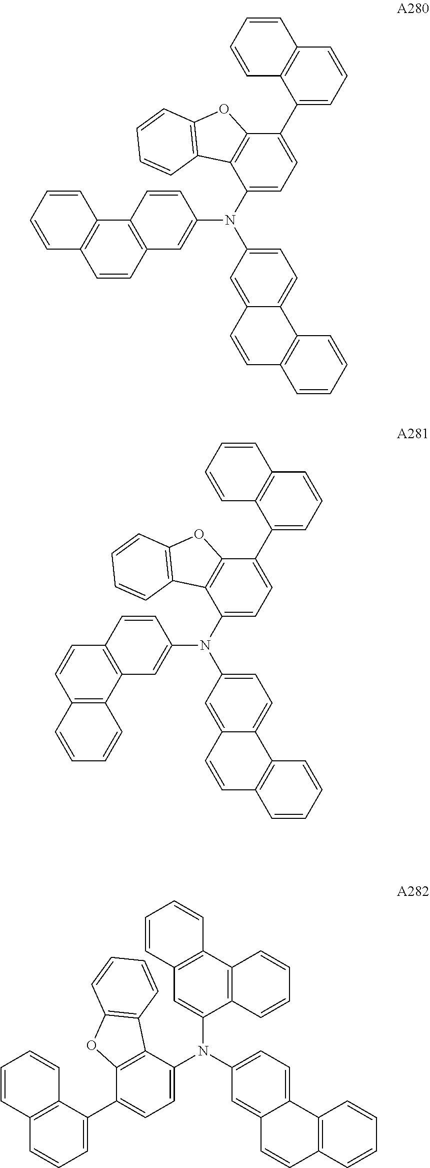 [SMILES: c1ccc2c(-c3ccc(N(c4ccc5c(ccc6ccccc65)c4)c4ccc5c(ccc6ccccc65)c4)c4c3oc3ccccc34)cccc2c1 ; c1ccc2c(-c3ccc(N(c4ccc5c(ccc6ccccc65)c4)c4ccc5ccc6ccccc6c5c4)c4c3oc3ccccc34)cccc2c1 ; c1ccc2c(-c3ccc(N(c4ccc5c(ccc6ccccc65)c4)c4cc5ccccc5c5ccccc45)c4c3oc3ccccc34)cccc2c1]

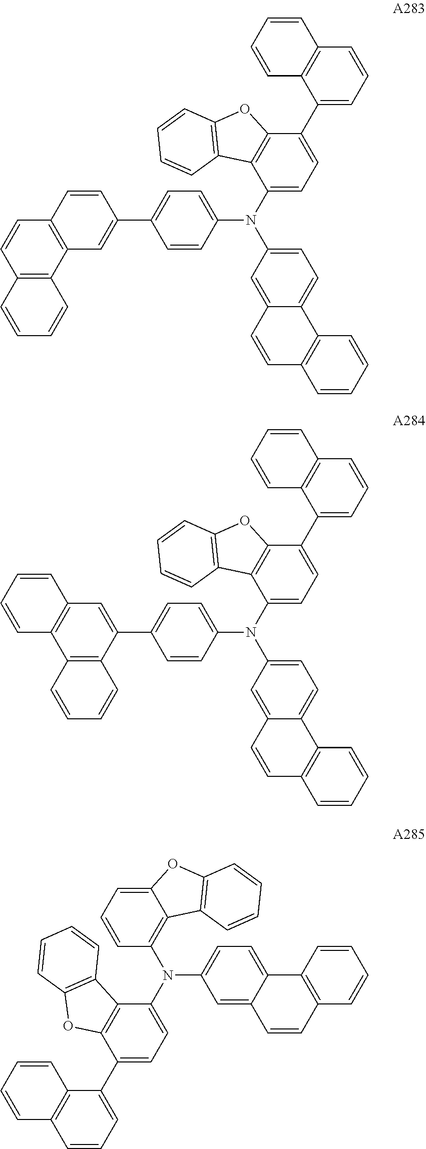

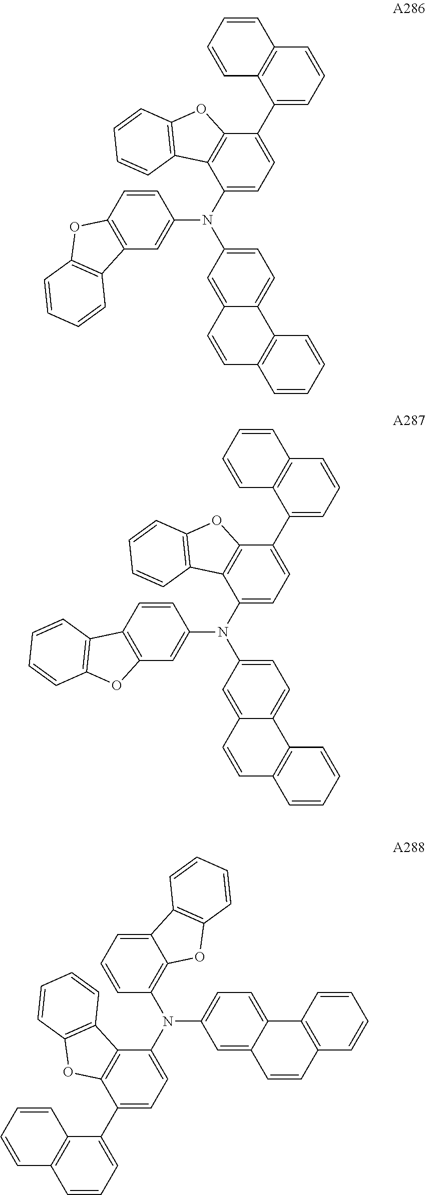

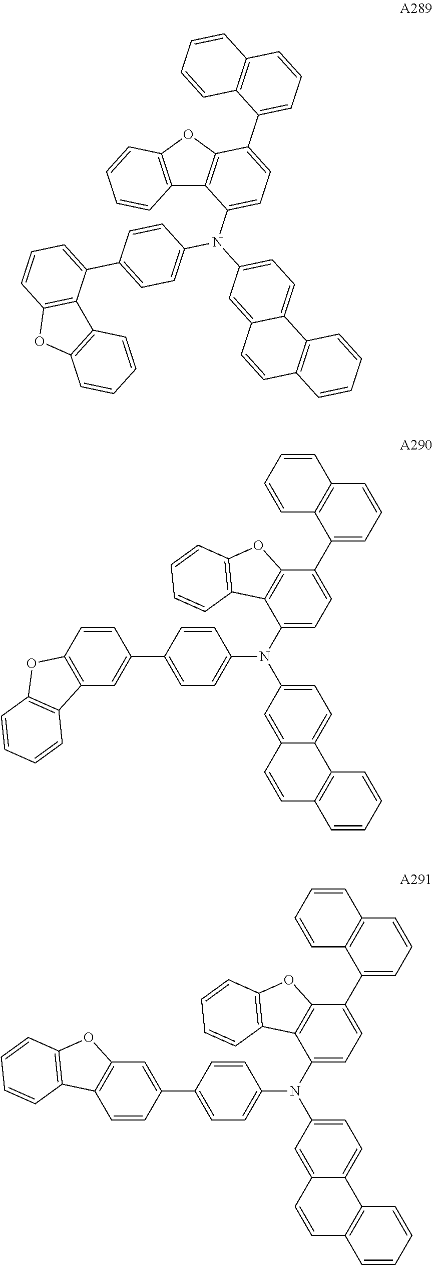 [SMILES: c1ccc2c(-c3ccc(N(c4ccc(-c5cccc6oc7ccccc7c56)cc4)c4ccc5c(ccc6ccccc65)c4)c4c3oc3ccccc34)cccc2c1 ; c1ccc2c(-c3ccc(N(c4ccc(-c5ccc6oc7ccccc7c6c5)cc4)c4ccc5c(ccc6ccccc65)c4)c4c3oc3ccccc34)cccc2c1 ; c1ccc2c(-c3ccc(N(c4ccc(-c5ccc6c(c5)oc5ccccc56)cc4)c4ccc5c(ccc6ccccc65)c4)c4c3oc3ccccc34)cccc2c1]

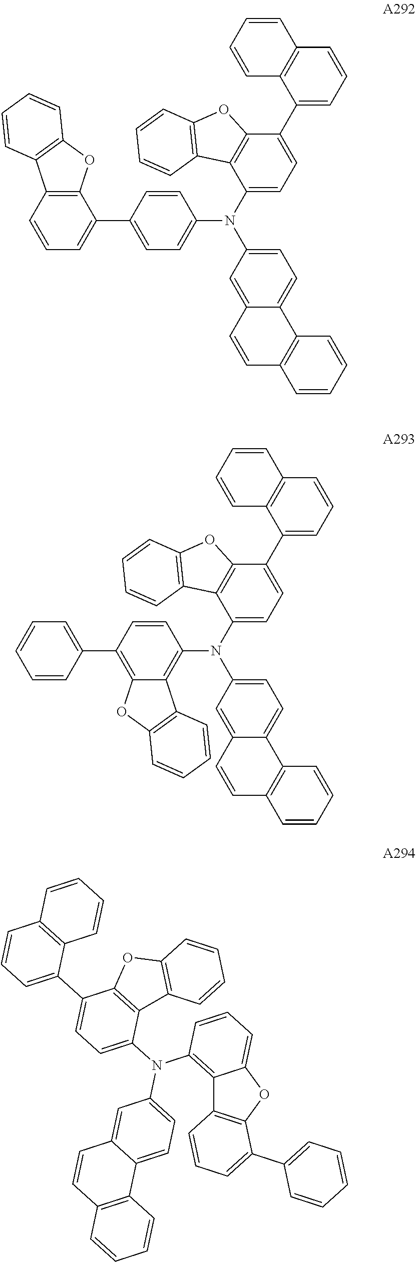 [SMILES: c1ccc2c(-c3ccc(N(c4ccc(-c5cccc6c5oc5ccccc56)cc4)c4ccc5c(ccc6ccccc65)c4)c4c3oc3ccccc34)cccc2c1 ; c1ccc(-c2ccc(N(c3ccc4c(ccc5ccccc54)c3)c3ccc(-c4cccc5ccccc45)c4oc5ccccc5c34)c3c2oc2ccccc23)cc1 ; c1ccc(-c2cccc3c2oc2cccc(N(c4ccc5c(ccc6ccccc65)c4)c4ccc(-c5cccc6ccccc56)c5oc6ccccc6c45)c23)cc1]

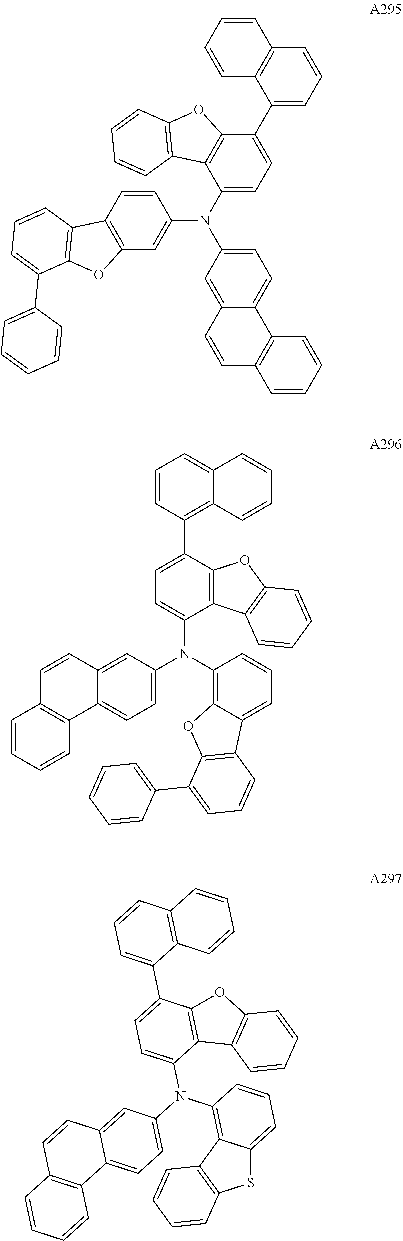 [SMILES: c1ccc(-c2cccc3c2oc2cc(N(c4ccc5c(ccc6ccccc65)c4)c4ccc(-c5cccc6ccccc56)c5oc6ccccc6c45)ccc23)cc1 ; c1ccc(-c2cccc3c2oc2c(N(c4ccc5c(ccc6ccccc65)c4)c4ccc(-c5cccc6ccccc56)c5oc6ccccc6c45)cccc23)cc1 ; c1ccc2c(-c3ccc(N(c4ccc5c(ccc6ccccc65)c4)c4cccc5sc6ccccc6c45)c4c3oc3ccccc34)cccc2c1]

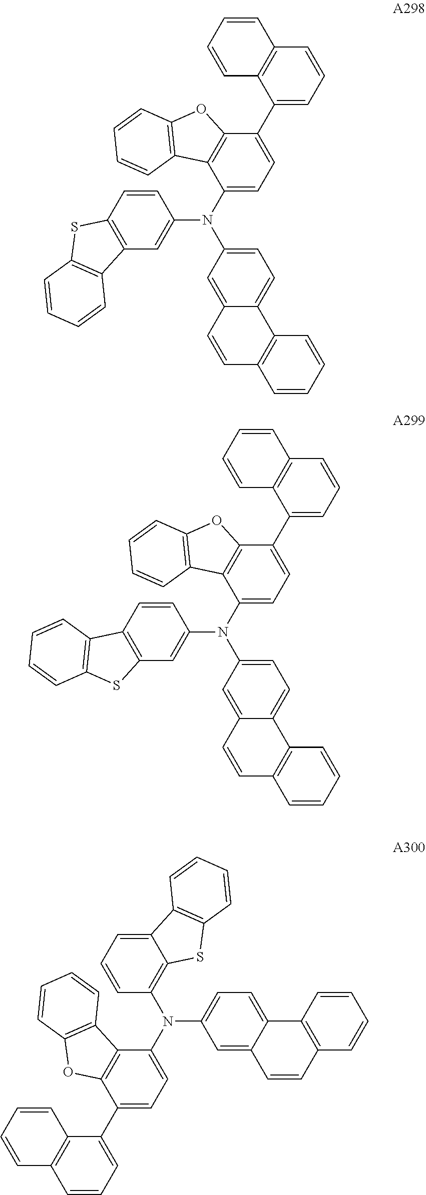

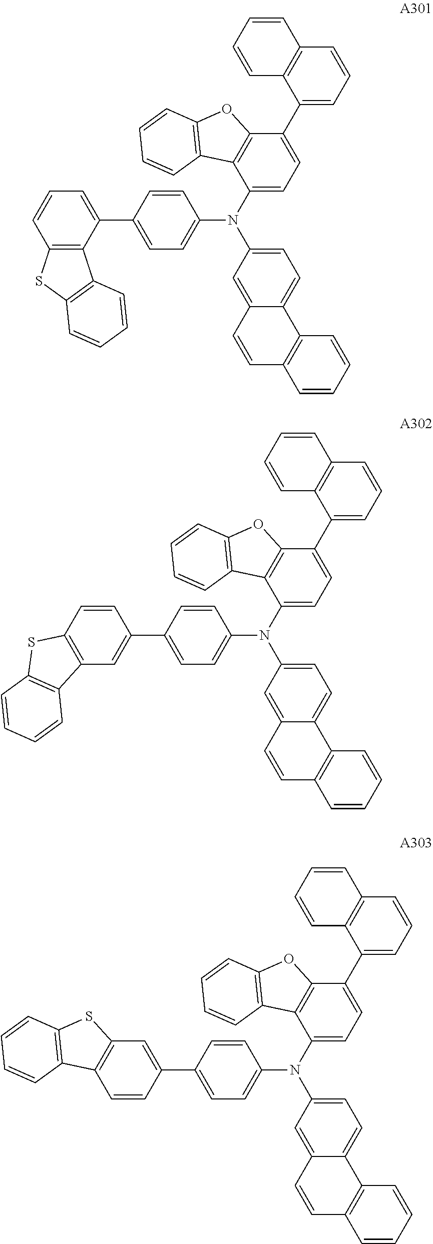

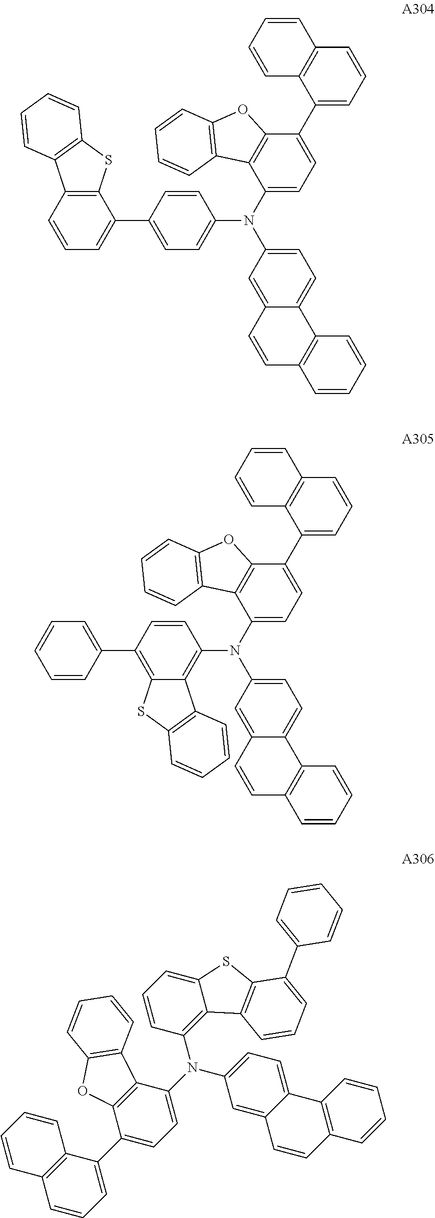 [SMILES: c1ccc2c(-c3ccc(N(c4ccc(-c5cccc6c5sc5ccccc56)cc4)c4ccc5c(ccc6ccccc65)c4)c4c3oc3ccccc34)cccc2c1 ; c1ccc(-c2ccc(N(c3ccc4c(ccc5ccccc54)c3)c3ccc(-c4cccc5ccccc45)c4oc5ccccc5c34)c3c2sc2ccccc23)cc1 ; c1ccc(-c2cccc3c2sc2cccc(N(c4ccc5c(ccc6ccccc65)c4)c4ccc(-c5cccc6ccccc56)c5oc6ccccc6c45)c23)cc1]

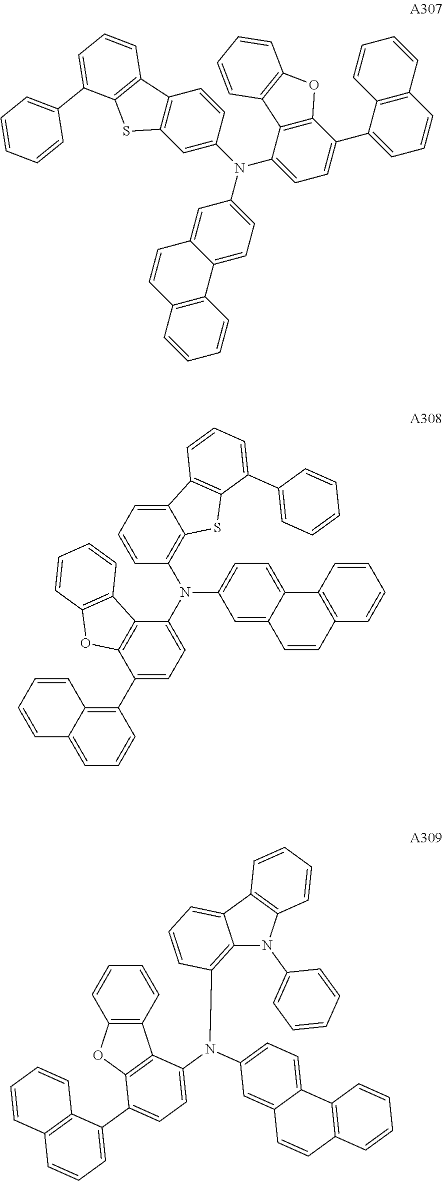 [SMILES: c1ccc(-c2cccc3c2sc2cc(N(c4ccc5c(ccc6ccccc65)c4)c4ccc(-c5cccc6ccccc56)c5oc6ccccc6c45)ccc23)cc1 ; c1ccc(-c2cccc3c2sc2c(N(c4ccc5c(ccc6ccccc65)c4)c4ccc(-c5cccc6ccccc56)c5oc6ccccc6c45)cccc23)cc1 ; c1ccc(-n2c3ccccc3c3cccc(N(c4ccc5c(ccc6ccccc65)c4)c4ccc(-c5cccc6ccccc56)c5oc6ccccc6c45)c32)cc1]

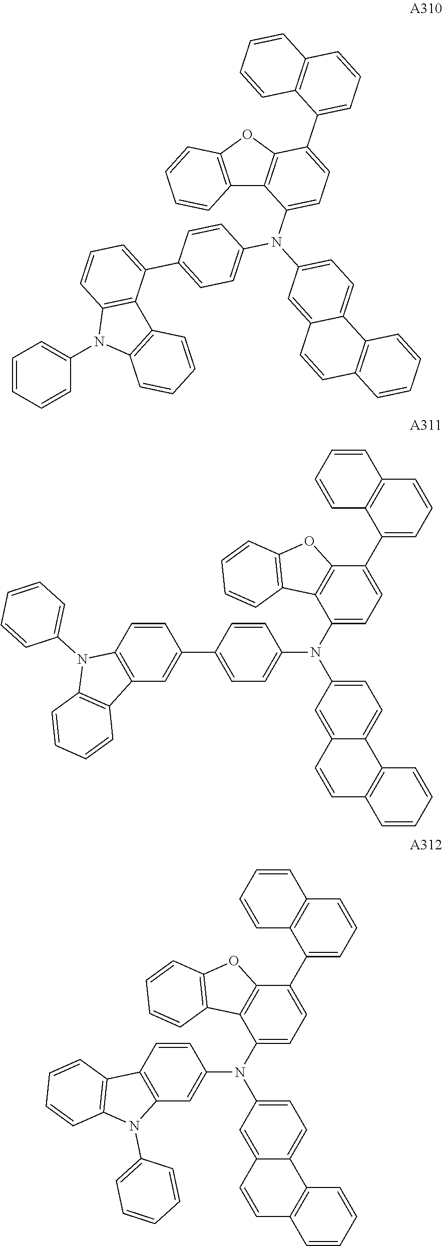 [SMILES: c1ccc(-n2c3ccccc3c3c(-c4ccc(N(c5ccc6c(ccc7ccccc76)c5)c5ccc(-c6cccc7ccccc67)c6oc7ccccc7c56)cc4)cccc32)cc1 ; c1ccc(-n2c3ccccc3c3cc(-c4ccc(N(c5ccc6c(ccc7ccccc76)c5)c5ccc(-c6cccc7ccccc67)c6oc7ccccc7c56)cc4)ccc32)cc1 ; c1ccc(-n2c3ccccc3c3ccc(N(c4ccc5c(ccc6ccccc65)c4)c4ccc(-c5cccc6ccccc56)c5oc6ccccc6c45)cc32)cc1]

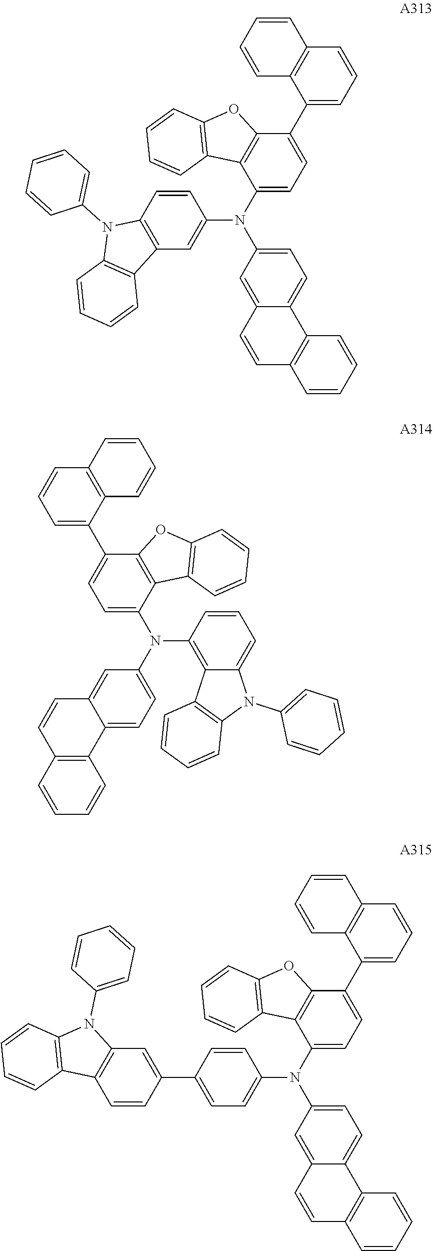 [SMILES: c1ccc(-n2c3ccccc3c3cc(N(c4ccc5c(ccc6ccccc65)c4)c4ccc(-c5cccc6ccccc56)c5oc6ccccc6c45)ccc32)cc1 ; c1ccc(-n2c3ccccc3c3c(N(c4ccc5c(ccc6ccccc65)c4)c4ccc(-c5cccc6ccccc56)c5oc6ccccc6c45)cccc32)cc1 ; c1ccc(-n2c3ccccc3c3ccc(-c4ccc(N(c5ccc6c(ccc7ccccc76)c5)c5ccc(-c6cccc7ccccc67)c6oc7ccccc7c56)cc4)cc32)cc1]

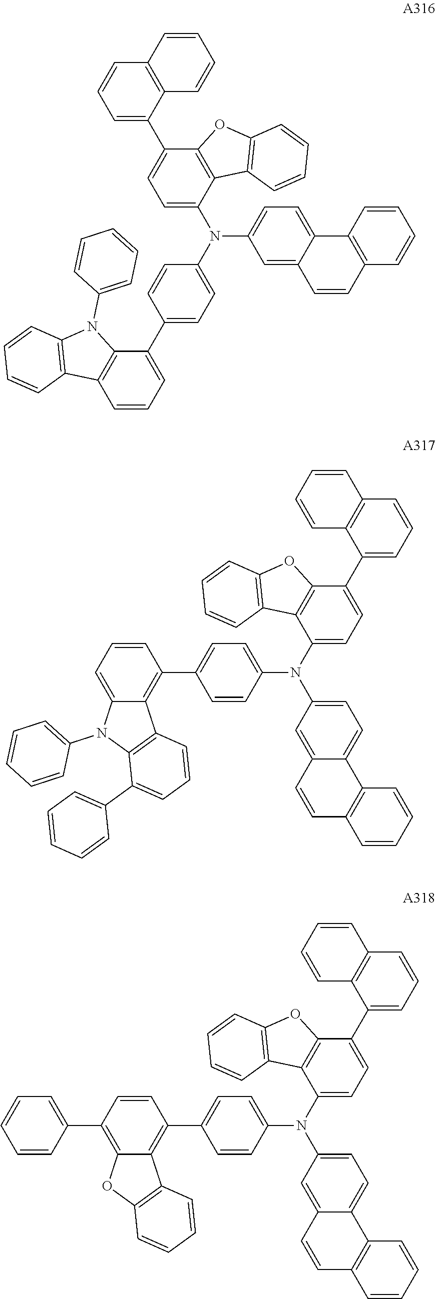 [SMILES: c1ccc(-n2c3ccccc3c3cccc(-c4ccc(N(c5ccc6c(ccc7ccccc76)c5)c5ccc(-c6cccc7ccccc67)c6oc7ccccc7c56)cc4)c32)cc1 ; c1ccc(-c2cccc3c4c(-c5ccc(N(c6ccc7c(ccc8ccccc87)c6)c6ccc(-c7cccc8ccccc78)c7oc8ccccc8c67)cc5)cccc4n(-c4ccccc4)c23)cc1 ; c1ccc(-c2ccc(-c3ccc(N(c4ccc5c(ccc6ccccc65)c4)c4ccc(-c5cccc6ccccc56)c5oc6ccccc6c45)cc3)c3c2oc2ccccc23)cc1]

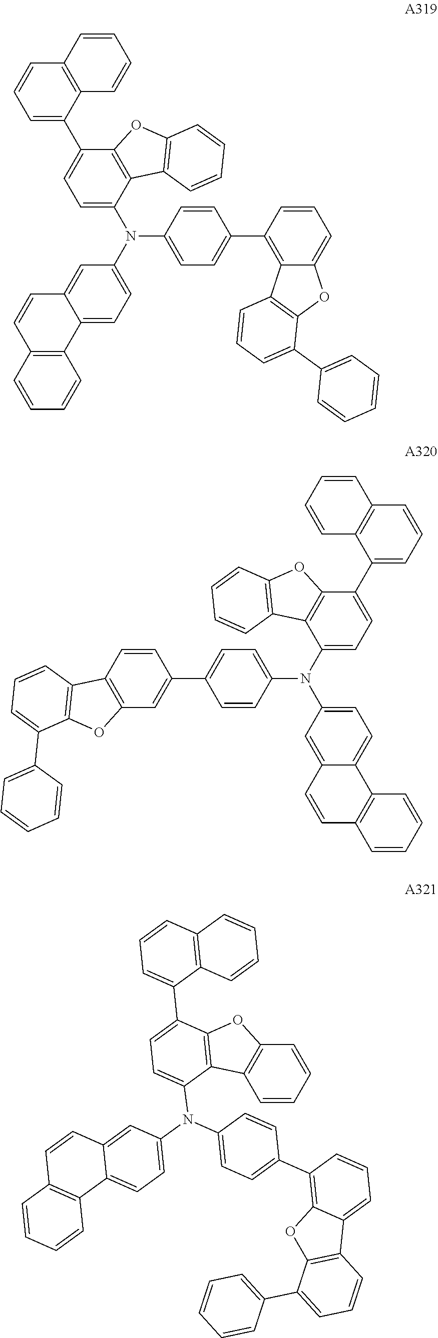 [SMILES: c1ccc(-c2cccc3c2oc2cccc(-c4ccc(N(c5ccc6c(ccc7ccccc76)c5)c5ccc(-c6cccc7ccccc67)c6oc7ccccc7c56)cc4)c23)cc1 ; c1ccc(-c2cccc3c2oc2cc(-c4ccc(N(c5ccc6c(ccc7ccccc76)c5)c5ccc(-c6cccc7ccccc67)c6oc7ccccc7c56)cc4)ccc23)cc1 ; c1ccc(-c2cccc3c2oc2c(-c4ccc(N(c5ccc6c(ccc7ccccc76)c5)c5ccc(-c6cccc7ccccc67)c6oc7ccccc7c56)cc4)cccc23)cc1]

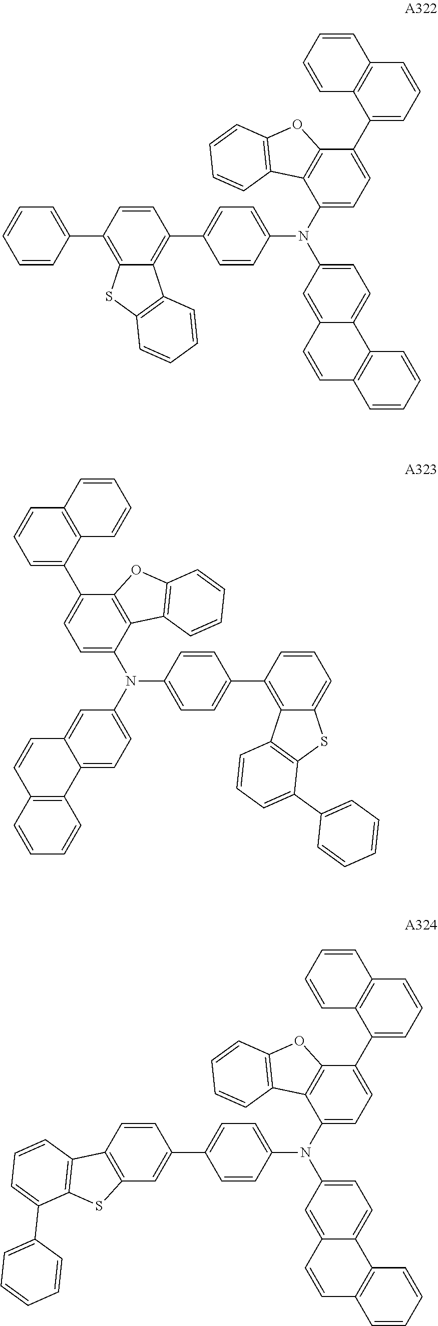 [SMILES: c1ccc(-c2ccc(-c3ccc(N(c4ccc5c(ccc6ccccc65)c4)c4ccc(-c5cccc6ccccc56)c5oc6ccccc6c45)cc3)c3c2sc2ccccc23)cc1 ; c1ccc(-c2cccc3c2sc2cccc(-c4ccc(N(c5ccc6c(ccc7ccccc76)c5)c5ccc(-c6cccc7ccccc67)c6oc7ccccc7c56)cc4)c23)cc1 ; c1ccc(-c2cccc3c2sc2cc(-c4ccc(N(c5ccc6c(ccc7ccccc76)c5)c5ccc(-c6cccc7ccccc67)c6oc7ccccc7c56)cc4)ccc23)cc1]

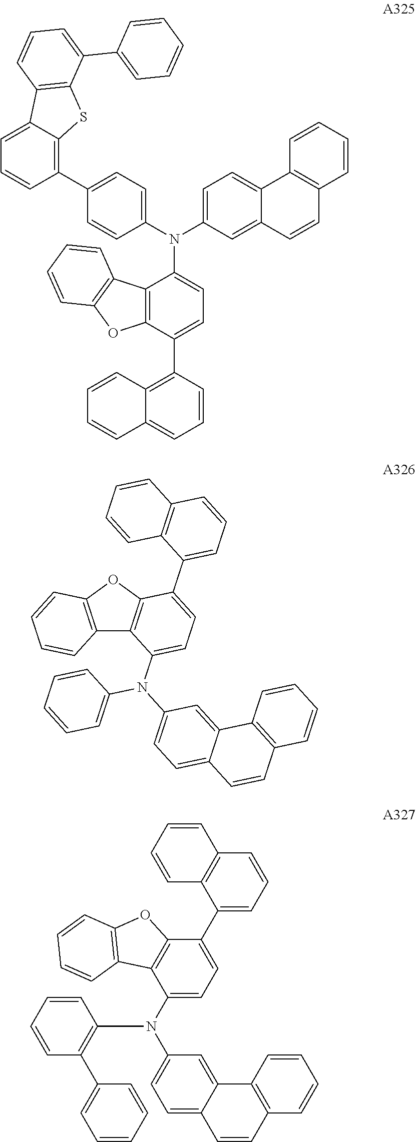 [SMILES: c1ccc(-c2cccc3c2sc2c(-c4ccc(N(c5ccc6c(ccc7ccccc76)c5)c5ccc(-c6cccc7ccccc67)c6oc7ccccc7c56)cc4)cccc23)cc1 ; c1ccc(N(c2ccc3ccc4ccccc4c3c2)c2ccc(-c3cccc4ccccc34)c3oc4ccccc4c23)cc1 ; c1ccc(-c2ccccc2N(c2ccc3ccc4ccccc4c3c2)c2ccc(-c3cccc4ccccc34)c3oc4ccccc4c23)cc1]

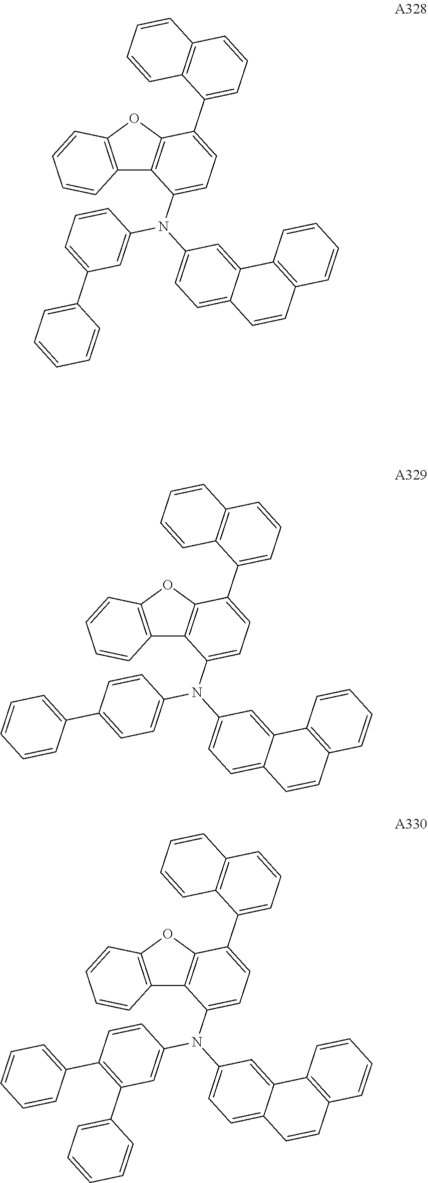 [SMILES: c1ccc(-c2cccc(N(c3ccc4ccc5ccccc5c4c3)c3ccc(-c4cccc5ccccc45)c4oc5ccccc5c34)c2)cc1 ; c1ccc(-c2ccc(N(c3ccc4ccc5ccccc5c4c3)c3ccc(-c4cccc5ccccc45)c4oc5ccccc5c34)cc2)cc1 ; c1ccc(-c2ccc(N(c3ccc4ccc5ccccc5c4c3)c3ccc(-c4cccc5ccccc45)c4oc5ccccc5c34)cc2-c2ccccc2)cc1]

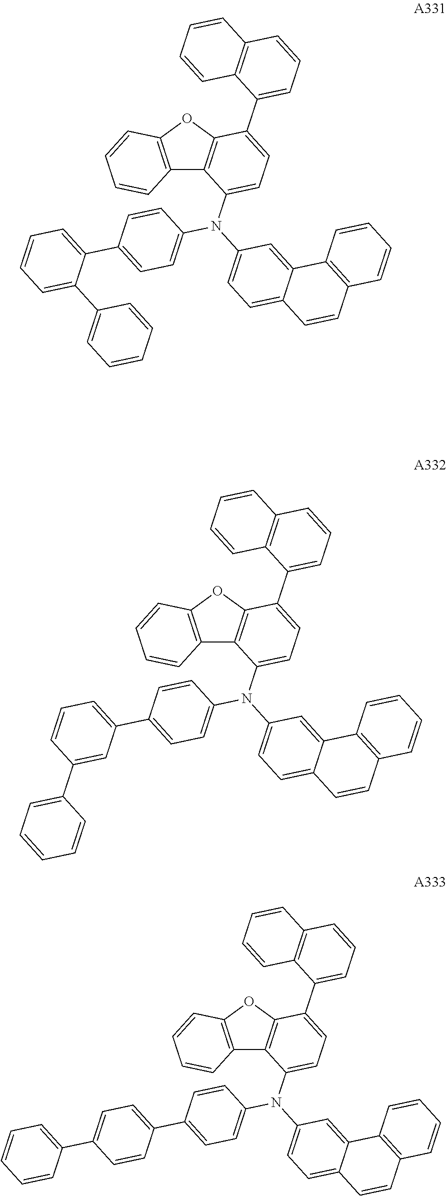 [SMILES: c1ccc(-c2ccccc2-c2ccc(N(c3ccc4ccc5ccccc5c4c3)c3ccc(-c4cccc5ccccc45)c4oc5ccccc5c34)cc2)cc1 ; c1ccc(-c2cccc(-c3ccc(N(c4ccc5ccc6ccccc6c5c4)c4ccc(-c5cccc6ccccc56)c5oc6ccccc6c45)cc3)c2)cc1 ; c1ccc(-c2ccc(-c3ccc(N(c4ccc5ccc6ccccc6c5c4)c4ccc(-c5cccc6ccccc56)c5oc6ccccc6c45)cc3)cc2)cc1]

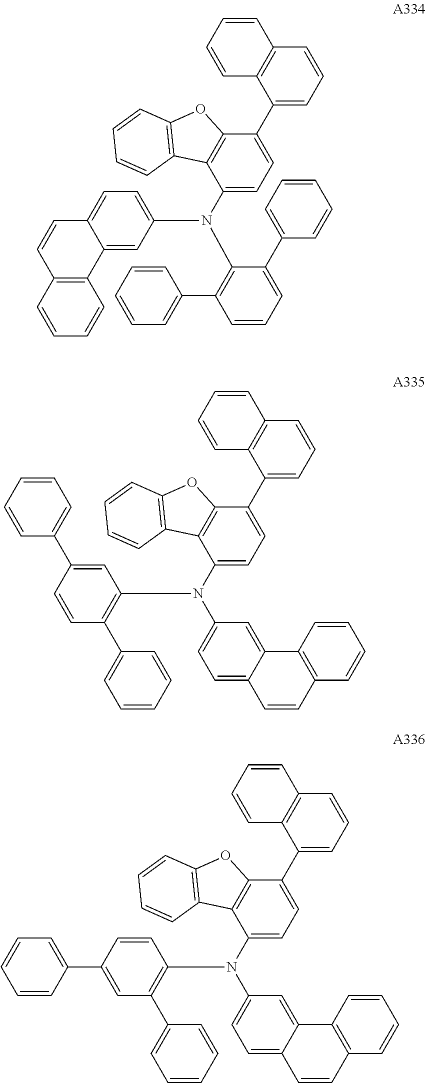 [SMILES: c1ccc(-c2cccc(-c3ccccc3)c2N(c2ccc3ccc4ccccc4c3c2)c2ccc(-c3cccc4ccccc34)c3oc4ccccc4c23)cc1 ; c1ccc(-c2ccc(-c3ccccc3)c(N(c3ccc4ccc5ccccc5c4c3)c3ccc(-c4cccc5ccccc45)c4oc5ccccc5c34)c2)cc1 ; c1ccc(-c2ccc(N(c3ccc4ccc5ccccc5c4c3)c3ccc(-c4cccc5ccccc45)c4oc5ccccc5c34)c(-c3ccccc3)c2)cc1]

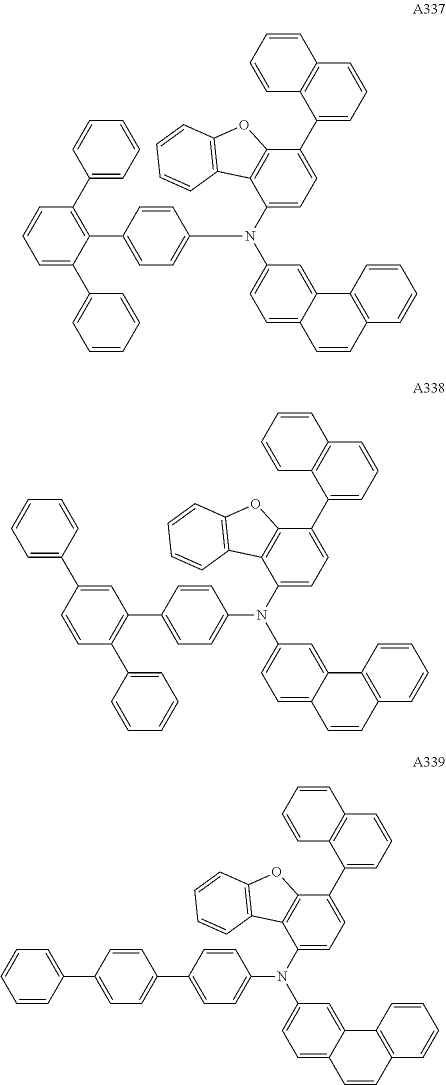 [SMILES: c1ccc(-c2cccc(-c3ccccc3)c2-c2ccc(N(c3ccc4ccc5ccccc5c4c3)c3ccc(-c4cccc5ccccc45)c4oc5ccccc5c34)cc2)cc1 ; c1ccc(-c2ccc(-c3ccccc3)c(-c3ccc(N(c4ccc5ccc6ccccc6c5c4)c4ccc(-c5cccc6ccccc56)c5oc6ccccc6c45)cc3)c2)cc1 ; c1ccc(-c2ccc(-c3ccc(N(c4ccc5ccc6ccccc6c5c4)c4ccc(-c5cccc6ccccc56)c5oc6ccccc6c45)cc3)cc2)cc1]

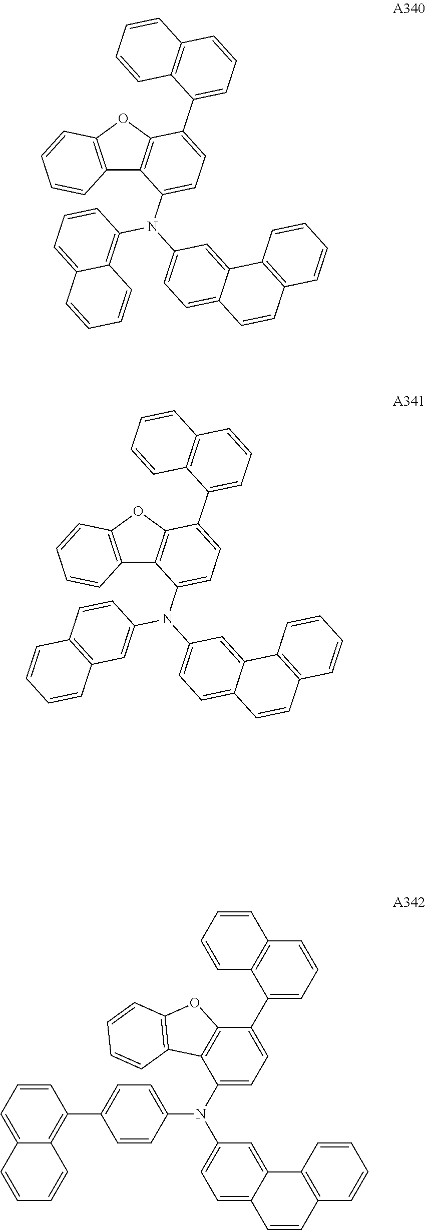 [SMILES: c1ccc2c(-c3ccc(N(c4ccc5ccc6ccccc6c5c4)c4cccc5ccccc45)c4c3oc3ccccc34)cccc2c1 ; c1ccc2cc(N(c3ccc4ccc5ccccc5c4c3)c3ccc(-c4cccc5ccccc45)c4oc5ccccc5c34)ccc2c1 ; c1ccc2c(-c3ccc(N(c4ccc5ccc6ccccc6c5c4)c4ccc(-c5cccc6ccccc56)c5oc6ccccc6c45)cc3)cccc2c1]

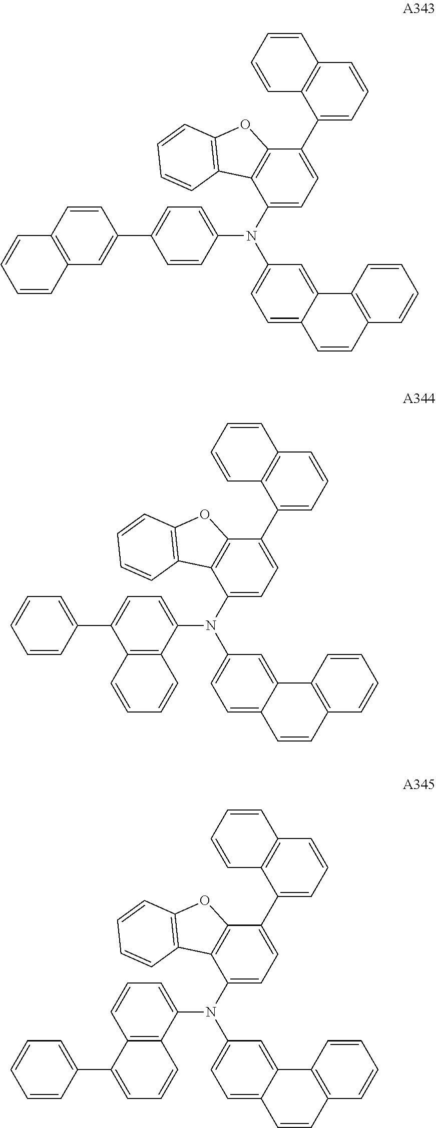 [SMILES: c1ccc2cc(-c3ccc(N(c4ccc5ccc6ccccc6c5c4)c4ccc(-c5cccc6ccccc56)c5oc6ccccc6c45)cc3)ccc2c1 ; c1ccc(-c2ccc(N(c3ccc4ccc5ccccc5c4c3)c3ccc(-c4cccc5ccccc45)c4oc5ccccc5c34)c3ccccc23)cc1 ; c1ccc(-c2cccc3c(N(c4ccc5ccc6ccccc6c5c4)c4ccc(-c5cccc6ccccc56)c5oc6ccccc6c45)cccc23)cc1]

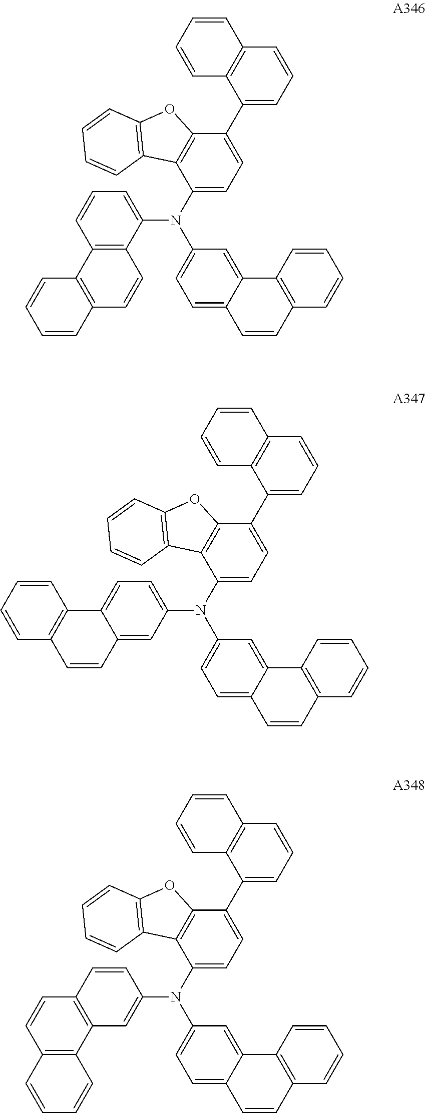 [SMILES: c1ccc2c(-c3ccc(N(c4ccc5ccc6ccccc6c5c4)c4cccc5c4ccc4ccccc45)c4c3oc3ccccc34)cccc2c1 ; c1ccc2c(-c3ccc(N(c4ccc5c(ccc6ccccc65)c4)c4ccc5ccc6ccccc6c5c4)c4c3oc3ccccc34)cccc2c1 ; c1ccc2c(-c3ccc(N(c4ccc5ccc6ccccc6c5c4)c4ccc5ccc6ccccc6c5c4)c4c3oc3ccccc34)cccc2c1]

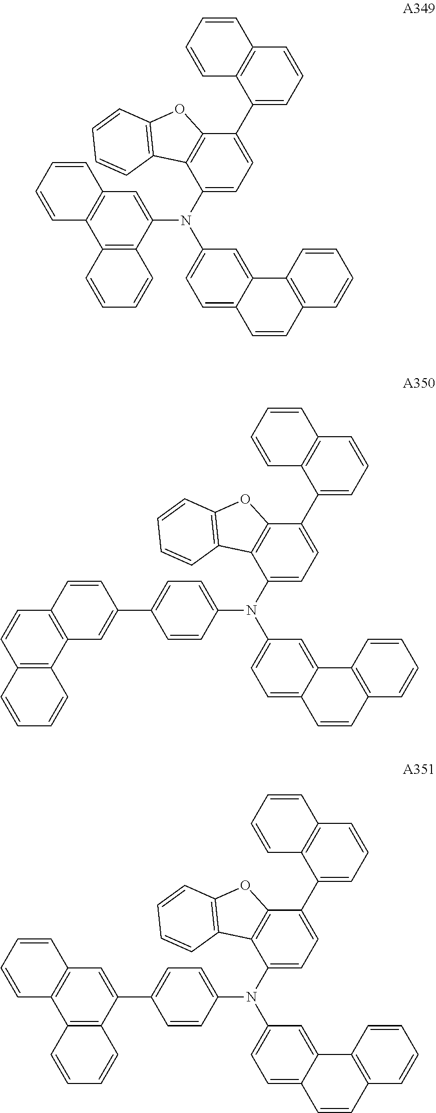 [SMILES: c1ccc2c(-c3ccc(N(c4ccc5ccc6ccccc6c5c4)c4cc5ccccc5c5ccccc45)c4c3oc3ccccc34)cccc2c1 ; c1ccc2c(-c3ccc(N(c4ccc(-c5ccc6ccc7ccccc7c6c5)cc4)c4ccc5ccc6ccccc6c5c4)c4c3oc3ccccc34)cccc2c1 ; c1ccc2c(-c3ccc(N(c4ccc(-c5cc6ccccc6c6ccccc56)cc4)c4ccc5ccc6ccccc6c5c4)c4c3oc3ccccc34)cccc2c1]

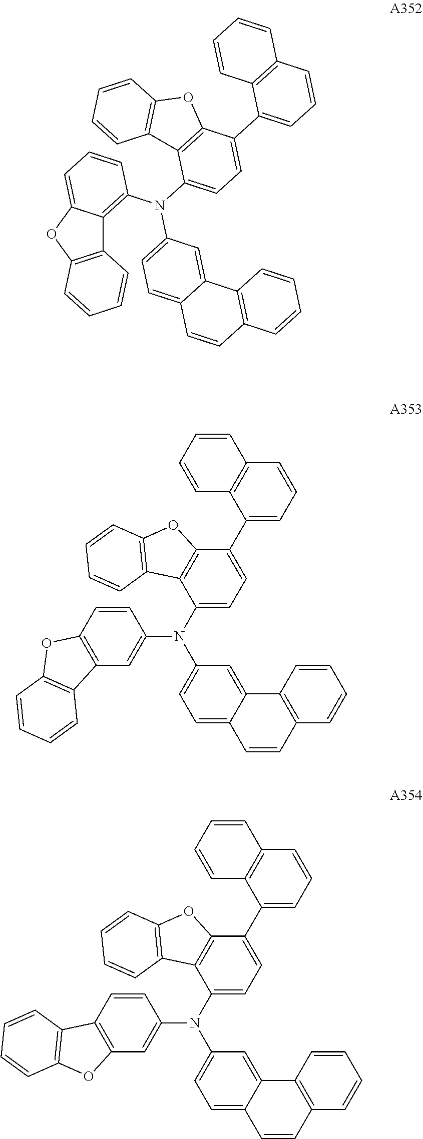 [SMILES: c1ccc2c(-c3ccc(N(c4ccc5ccc6ccccc6c5c4)c4cccc5oc6ccccc6c45)c4c3oc3ccccc34)cccc2c1 ; c1ccc2c(-c3ccc(N(c4ccc5ccc6ccccc6c5c4)c4ccc5oc6ccccc6c5c4)c4c3oc3ccccc34)cccc2c1 ; c1ccc2c(-c3ccc(N(c4ccc5c(c4)oc4ccccc45)c4ccc5ccc6ccccc6c5c4)c4c3oc3ccccc34)cccc2c1]

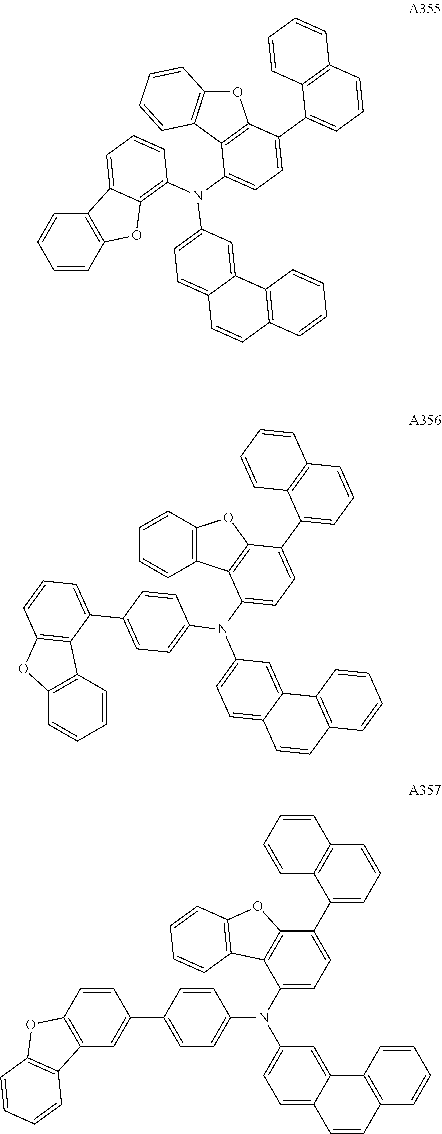 [SMILES: c1ccc2c(-c3ccc(N(c4ccc5ccc6ccccc6c5c4)c4cccc5c4oc4ccccc45)c4c3oc3ccccc34)cccc2c1 ; c1ccc2c(-c3ccc(N(c4ccc(-c5cccc6oc7ccccc7c56)cc4)c4ccc5ccc6ccccc6c5c4)c4c3oc3ccccc34)cccc2c1 ; c1ccc2c(-c3ccc(N(c4ccc(-c5ccc6oc7ccccc7c6c5)cc4)c4ccc5ccc6ccccc6c5c4)c4c3oc3ccccc34)cccc2c1]

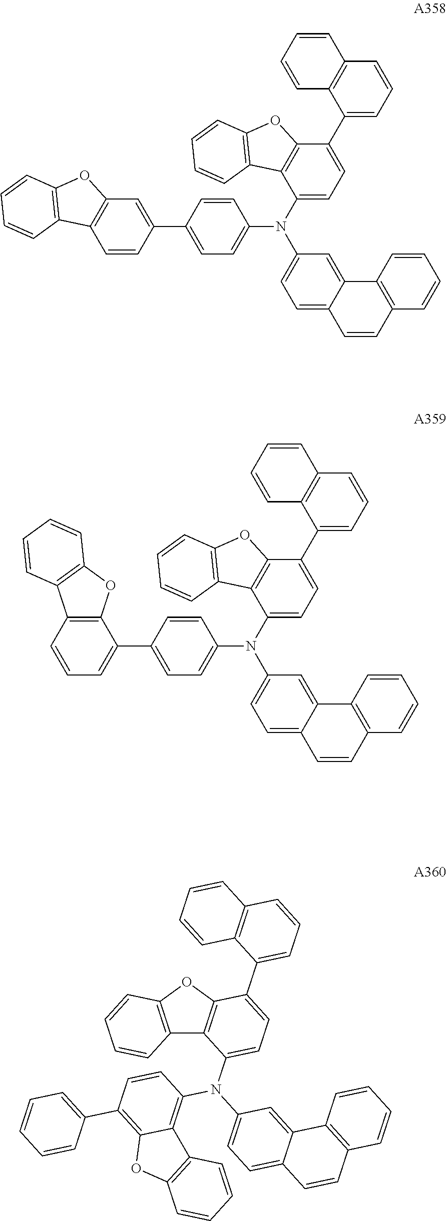 [SMILES: c1ccc2c(-c3ccc(N(c4ccc(-c5ccc6c(c5)oc5ccccc56)cc4)c4ccc5ccc6ccccc6c5c4)c4c3oc3ccccc34)cccc2c1 ; c1ccc2c(-c3ccc(N(c4ccc(-c5cccc6c5oc5ccccc56)cc4)c4ccc5ccc6ccccc6c5c4)c4c3oc3ccccc34)cccc2c1 ; c1ccc(-c2ccc(N(c3ccc4ccc5ccccc5c4c3)c3ccc(-c4cccc5ccccc45)c4oc5ccccc5c34)c3c2oc2ccccc23)cc1]

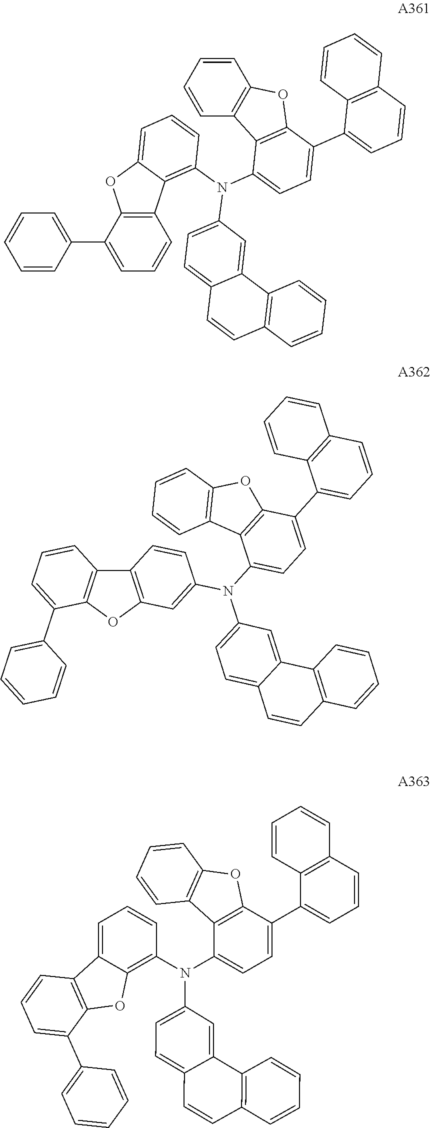 [SMILES: c1ccc(-c2cccc3c2oc2cccc(N(c4ccc5ccc6ccccc6c5c4)c4ccc(-c5cccc6ccccc56)c5oc6ccccc6c45)c23)cc1 ; c1ccc(-c2cccc3c2oc2cc(N(c4ccc5ccc6ccccc6c5c4)c4ccc(-c5cccc6ccccc56)c5oc6ccccc6c45)ccc23)cc1 ; c1ccc(-c2cccc3c2oc2c(N(c4ccc5ccc6ccccc6c5c4)c4ccc(-c5cccc6ccccc56)c5oc6ccccc6c45)cccc23)cc1]

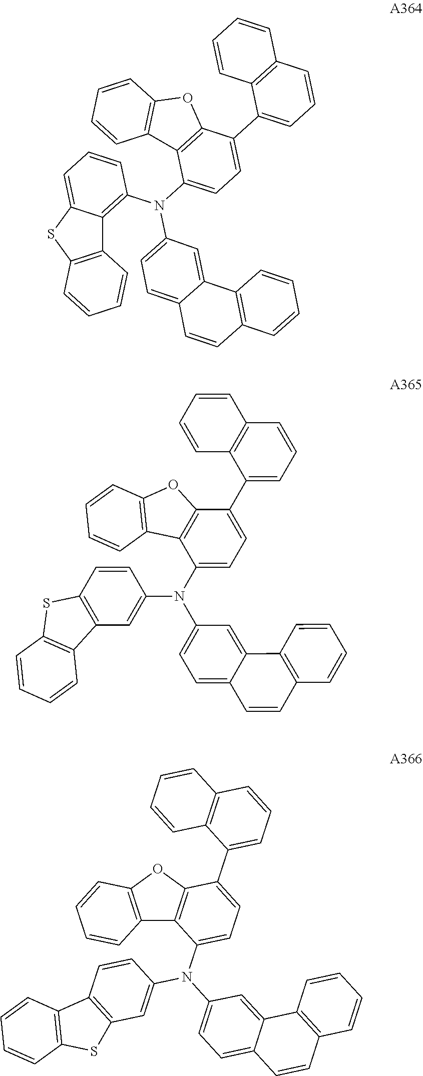 [SMILES: c1ccc2c(-c3ccc(N(c4ccc5ccc6ccccc6c5c4)c4cccc5sc6ccccc6c45)c4c3oc3ccccc34)cccc2c1 ; c1ccc2c(-c3ccc(N(c4ccc5ccc6ccccc6c5c4)c4ccc5sc6ccccc6c5c4)c4c3oc3ccccc34)cccc2c1 ; c1ccc2c(-c3ccc(N(c4ccc5c(c4)sc4ccccc45)c4ccc5ccc6ccccc6c5c4)c4c3oc3ccccc34)cccc2c1]

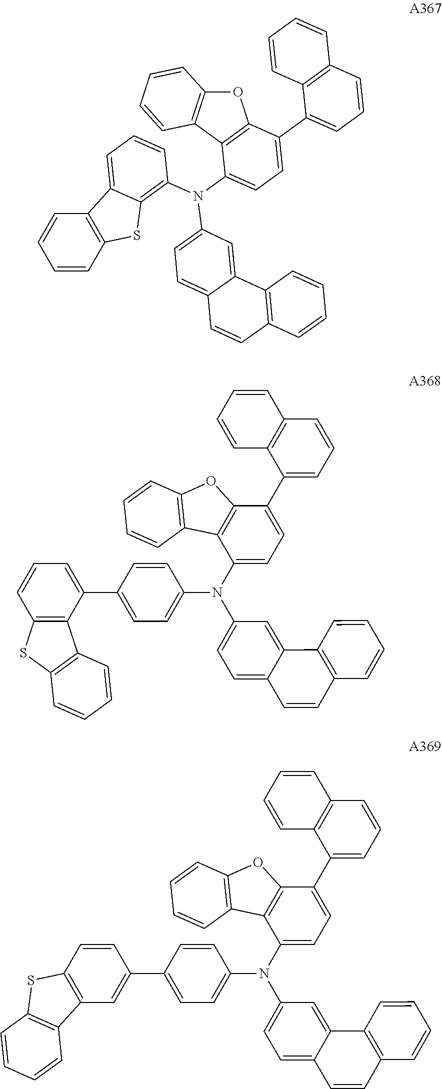 [SMILES: c1ccc2c(-c3ccc(N(c4ccc5ccc6ccccc6c5c4)c4cccc5c4sc4ccccc45)c4c3oc3ccccc34)cccc2c1 ; c1ccc2c(-c3ccc(N(c4ccc(-c5cccc6sc7ccccc7c56)cc4)c4ccc5ccc6ccccc6c5c4)c4c3oc3ccccc34)cccc2c1 ; c1ccc2c(-c3ccc(N(c4ccc(-c5ccc6sc7ccccc7c6c5)cc4)c4ccc5ccc6ccccc6c5c4)c4c3oc3ccccc34)cccc2c1]

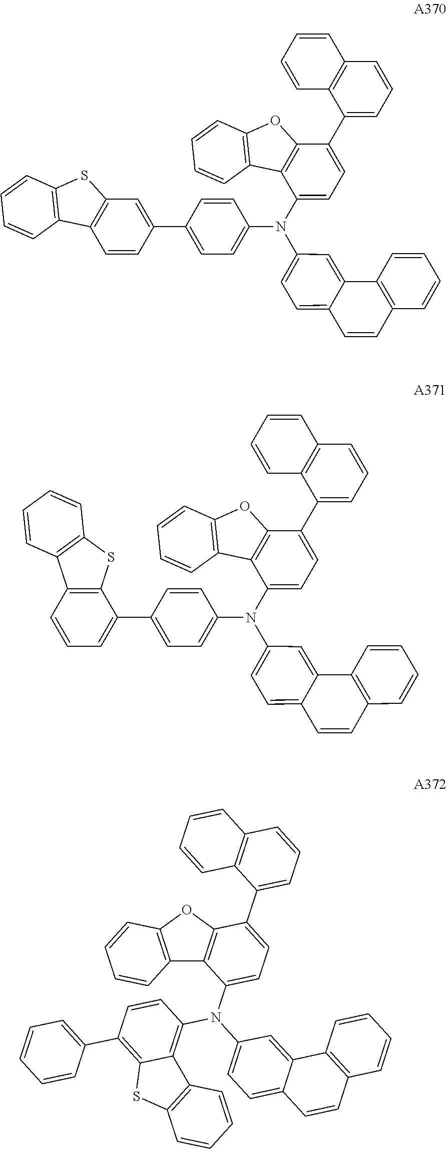 [SMILES: c1ccc2c(-c3ccc(N(c4ccc(-c5ccc6c(c5)sc5ccccc56)cc4)c4ccc5ccc6ccccc6c5c4)c4c3oc3ccccc34)cccc2c1 ; c1ccc2c(-c3ccc(N(c4ccc(-c5cccc6c5sc5ccccc56)cc4)c4ccc5ccc6ccccc6c5c4)c4c3oc3ccccc34)cccc2c1 ; c1ccc(-c2ccc(N(c3ccc4ccc5ccccc5c4c3)c3ccc(-c4cccc5ccccc45)c4oc5ccccc5c34)c3c2sc2ccccc23)cc1]

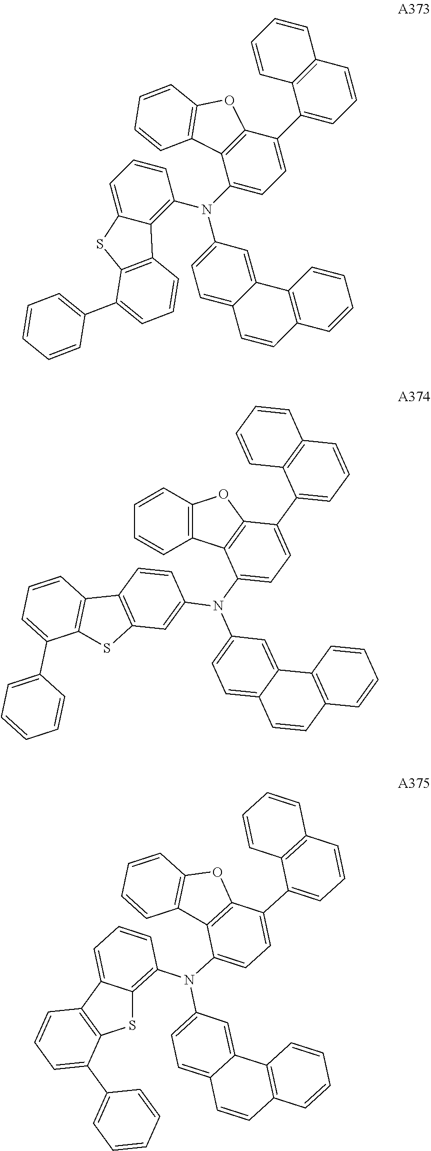 [SMILES: c1ccc(-c2cccc3c2sc2cccc(N(c4ccc5ccc6ccccc6c5c4)c4ccc(-c5cccc6ccccc56)c5oc6ccccc6c45)c23)cc1 ; c1ccc(-c2cccc3c2sc2cc(N(c4ccc5ccc6ccccc6c5c4)c4ccc(-c5cccc6ccccc56)c5oc6ccccc6c45)ccc23)cc1 ; c1ccc(-c2cccc3c2sc2c(N(c4ccc5ccc6ccccc6c5c4)c4ccc(-c5cccc6ccccc56)c5oc6ccccc6c45)cccc23)cc1]

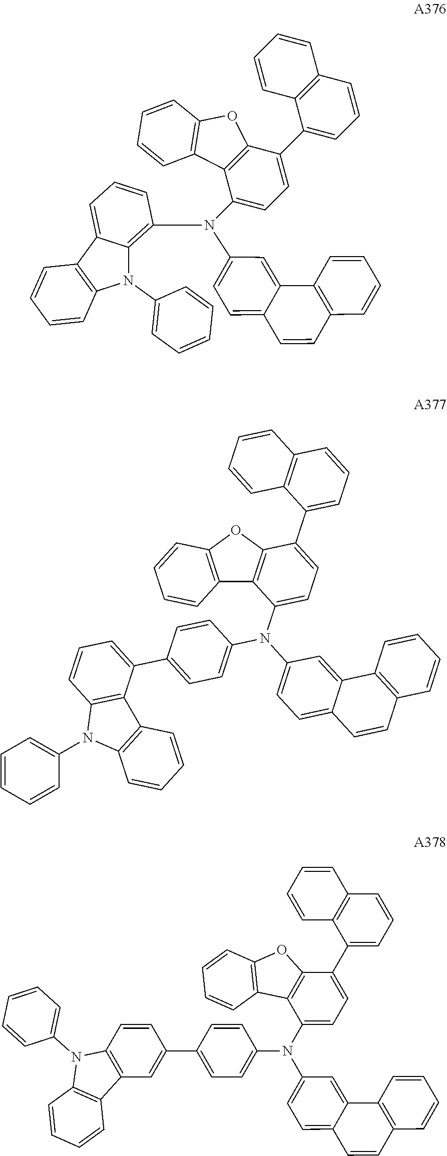 [SMILES: c1ccc(-n2c3ccccc3c3cccc(N(c4ccc5ccc6ccccc6c5c4)c4ccc(-c5cccc6ccccc56)c5oc6ccccc6c45)c32)cc1 ; c1ccc(-n2c3ccccc3c3c(-c4ccc(N(c5ccc6ccc7ccccc7c6c5)c5ccc(-c6cccc7ccccc67)c6oc7ccccc7c56)cc4)cccc32)cc1 ; c1ccc(-n2c3ccccc3c3cc(-c4ccc(N(c5ccc6ccc7ccccc7c6c5)c5ccc(-c6cccc7ccccc67)c6oc7ccccc7c56)cc4)ccc32)cc1]

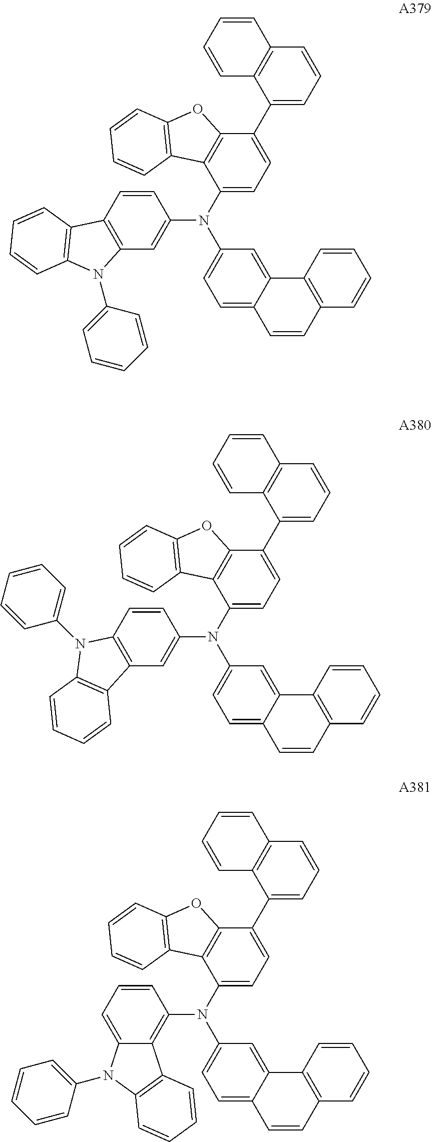 [SMILES: c1ccc(-n2c3ccccc3c3ccc(N(c4ccc5ccc6ccccc6c5c4)c4ccc(-c5cccc6ccccc56)c5oc6ccccc6c45)cc32)cc1 ; c1ccc(-n2c3ccccc3c3cc(N(c4ccc5ccc6ccccc6c5c4)c4ccc(-c5cccc6ccccc56)c5oc6ccccc6c45)ccc32)cc1 ; c1ccc(-n2c3ccccc3c3c(N(c4ccc5ccc6ccccc6c5c4)c4ccc(-c5cccc6ccccc56)c5oc6ccccc6c45)cccc32)cc1]

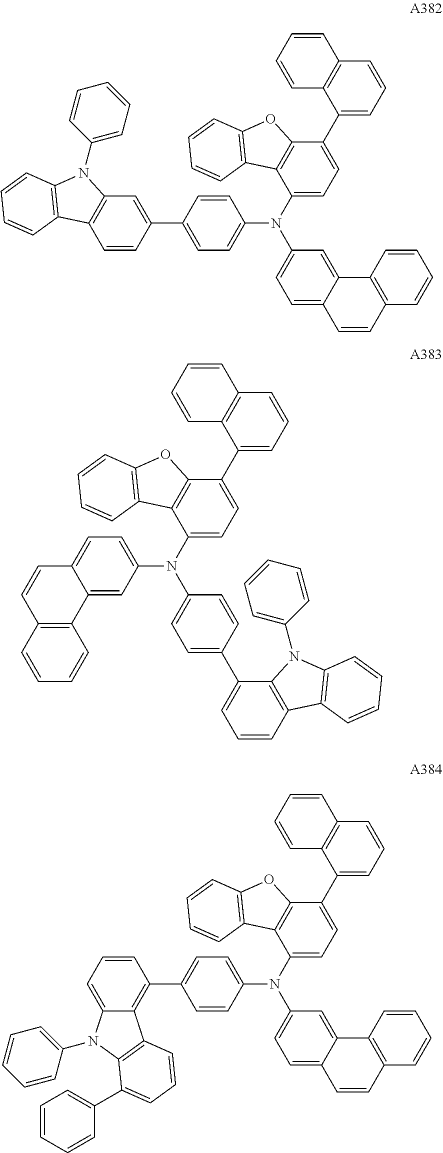 [SMILES: c1ccc(-n2c3ccccc3c3ccc(-c4ccc(N(c5ccc6ccc7ccccc7c6c5)c5ccc(-c6cccc7ccccc67)c6oc7ccccc7c56)cc4)cc32)cc1 ; c1ccc(-n2c3ccccc3c3cccc(-c4ccc(N(c5ccc6ccc7ccccc7c6c5)c5ccc(-c6cccc7ccccc67)c6oc7ccccc7c56)cc4)c32)cc1 ; c1ccc(-c2cccc3c4c(-c5ccc(N(c6ccc7ccc8ccccc8c7c6)c6ccc(-c7cccc8ccccc78)c7oc8ccccc8c67)cc5)cccc4n(-c4ccccc4)c23)cc1]

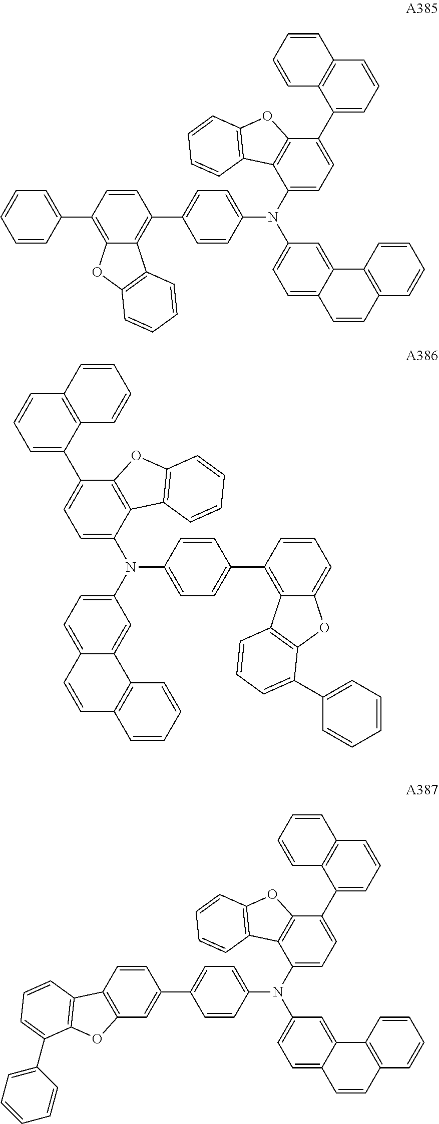 [SMILES: c1ccc(-c2ccc(-c3ccc(N(c4ccc5ccc6ccccc6c5c4)c4ccc(-c5cccc6ccccc56)c5oc6ccccc6c45)cc3)c3c2oc2ccccc23)cc1 ; c1ccc(-c2cccc3c2oc2cccc(-c4ccc(N(c5ccc6ccc7ccccc7c6c5)c5ccc(-c6cccc7ccccc67)c6oc7ccccc7c56)cc4)c23)cc1 ; c1ccc(-c2cccc3c2oc2cc(-c4ccc(N(c5ccc6ccc7ccccc7c6c5)c5ccc(-c6cccc7ccccc67)c6oc7ccccc7c56)cc4)ccc23)cc1]

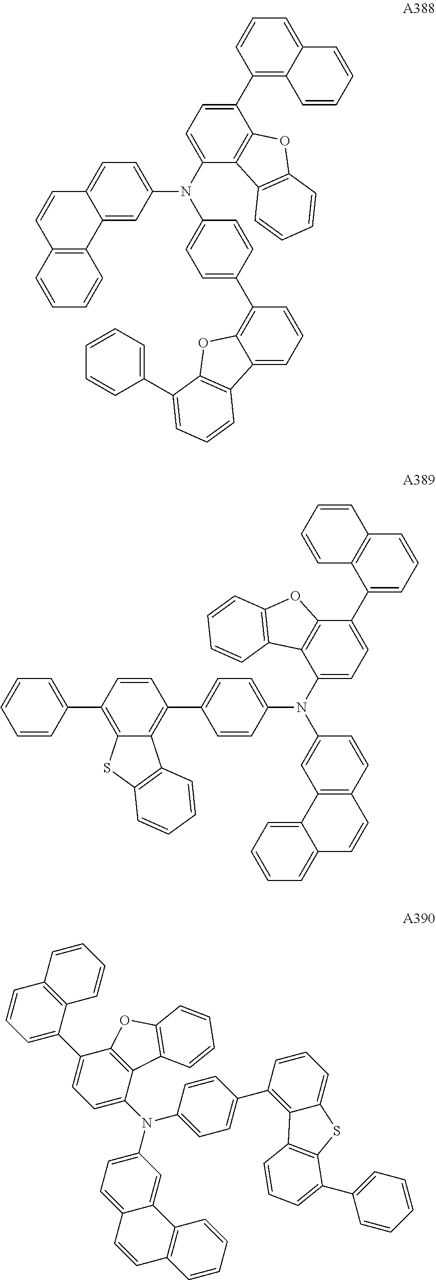 [SMILES: c1ccc(-c2cccc3c2oc2c(-c4ccc(N(c5ccc6ccc7ccccc7c6c5)c5ccc(-c6cccc7ccccc67)c6oc7ccccc7c56)cc4)cccc23)cc1 ; c1ccc(-c2ccc(-c3ccc(N(c4ccc5ccc6ccccc6c5c4)c4ccc(-c5cccc6ccccc56)c5oc6ccccc6c45)cc3)c3c2sc2ccccc23)cc1 ; c1ccc(-c2cccc3c2sc2cccc(-c4ccc(N(c5ccc6ccc7ccccc7c6c5)c5ccc(-c6cccc7ccccc67)c6oc7ccccc7c56)cc4)c23)cc1]

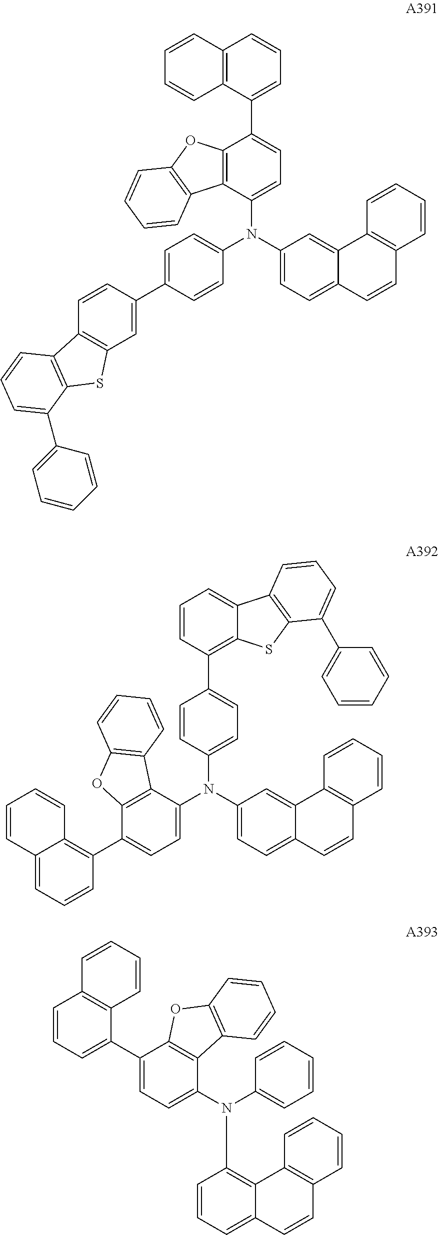 [SMILES: c1ccc(-c2cccc3c2sc2cc(-c4ccc(N(c5ccc6ccc7ccccc7c6c5)c5ccc(-c6cccc7ccccc67)c6oc7ccccc7c56)cc4)ccc23)cc1 ; c1ccc(-c2cccc3c2sc2c(-c4ccc(N(c5ccc6ccc7ccccc7c6c5)c5ccc(-c6cccc7ccccc67)c6oc7ccccc7c56)cc4)cccc23)cc1 ; c1ccc(N(c2cccc3ccc4ccccc4c23)c2ccc(-c3cccc4ccccc34)c3oc4ccccc4c23)cc1]

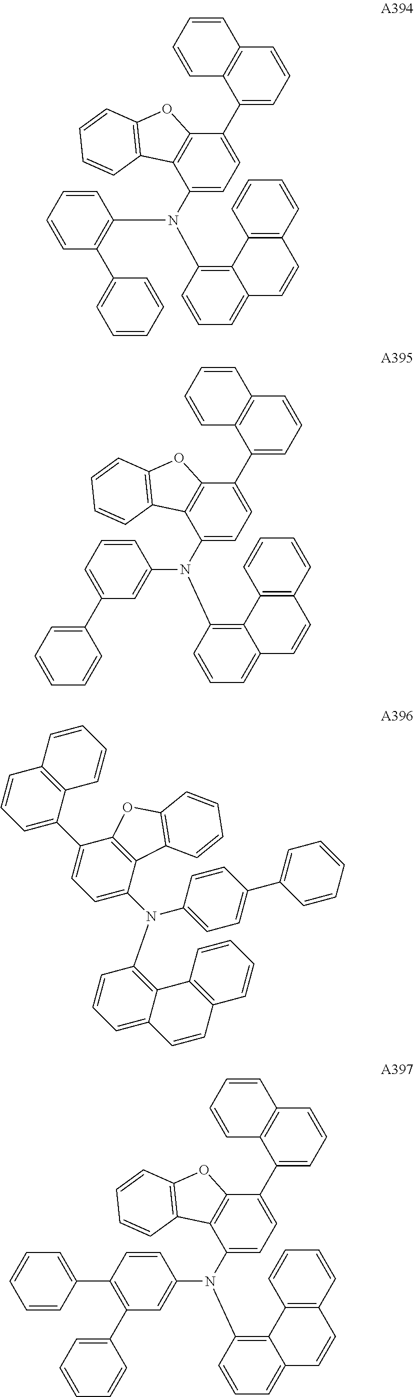

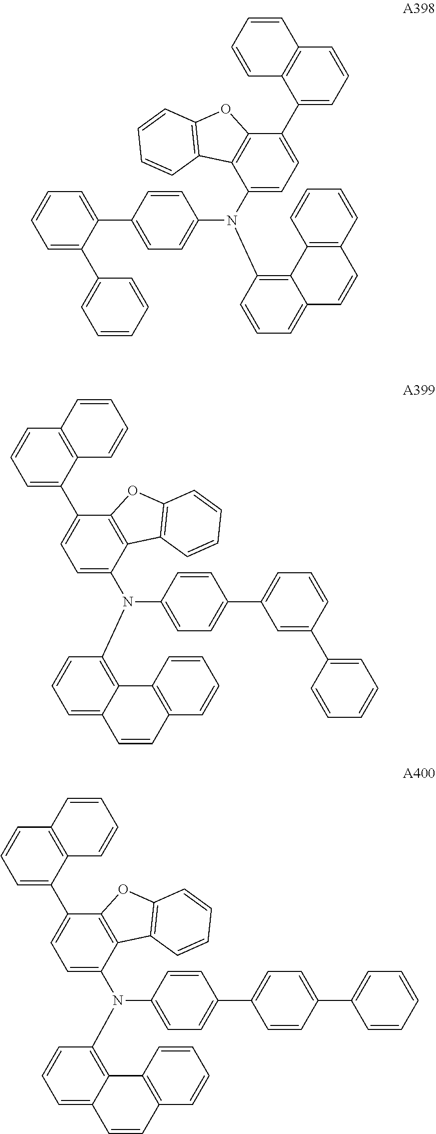 [SMILES: c1ccc(-c2ccccc2-c2ccc(N(c3cccc4ccc5ccccc5c34)c3ccc(-c4cccc5ccccc45)c4oc5ccccc5c34)cc2)cc1 ; c1ccc(-c2cccc(-c3ccc(N(c4cccc5ccc6ccccc6c45)c4ccc(-c5cccc6ccccc56)c5oc6ccccc6c45)cc3)c2)cc1 ; c1ccc(-c2ccc(-c3ccc(N(c4cccc5ccc6ccccc6c45)c4ccc(-c5cccc6ccccc56)c5oc6ccccc6c45)cc3)cc2)cc1]

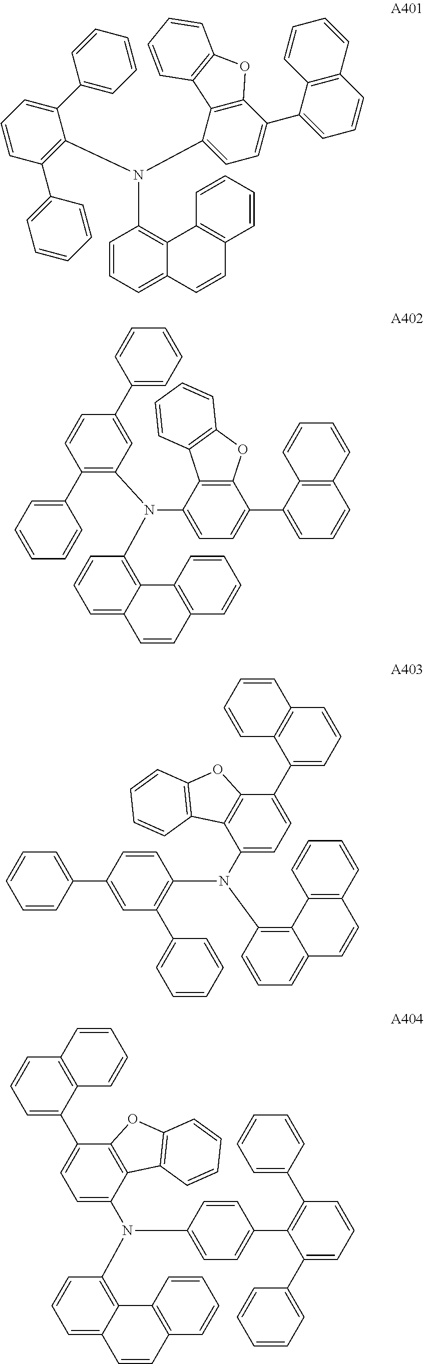

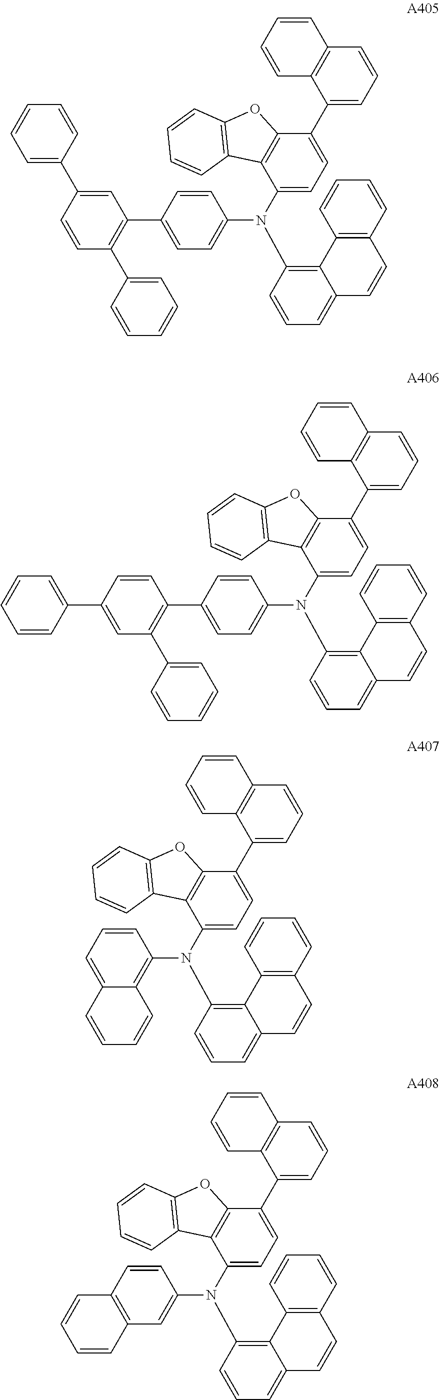

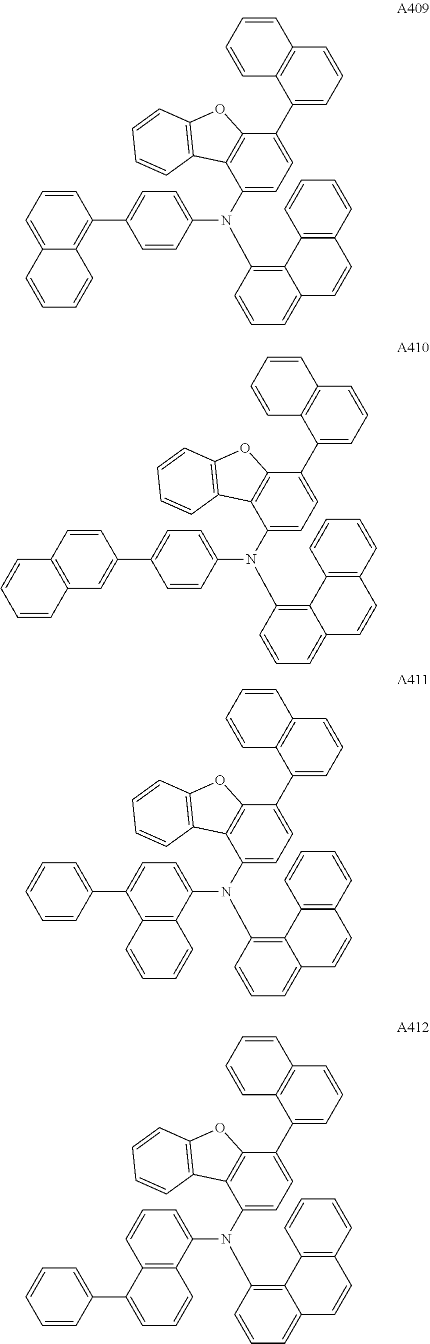

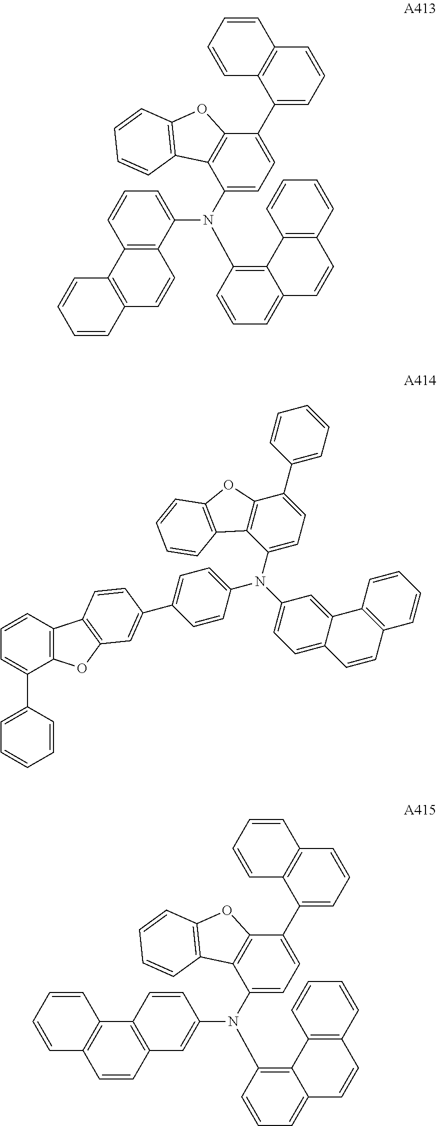 [SMILES: c1ccc2c(-c3ccc(N(c4cccc5c4ccc4ccccc45)c4cccc5ccc6ccccc6c45)c4c3oc3ccccc34)cccc2c1 ; c1ccc(-c2cccc3c2oc2cc(-c4ccc(N(c5ccc6ccc7ccccc7c6c5)c5ccc(-c6ccccc6)c6oc7ccccc7c56)cc4)ccc23)cc1 ; c1ccc2c(-c3ccc(N(c4ccc5c(ccc6ccccc65)c4)c4cccc5ccc6ccccc6c45)c4c3oc3ccccc34)cccc2c1]

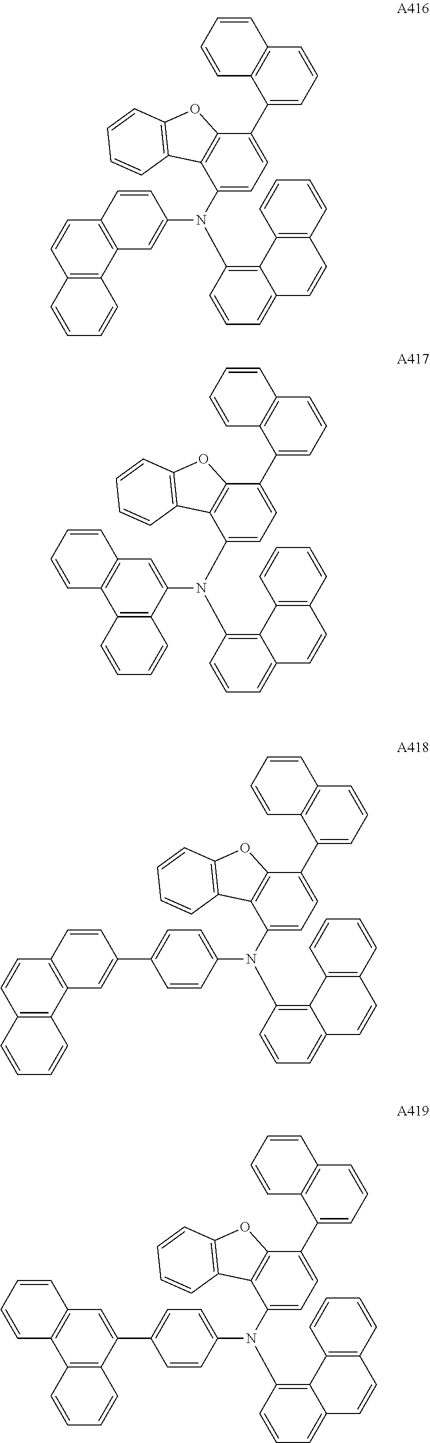

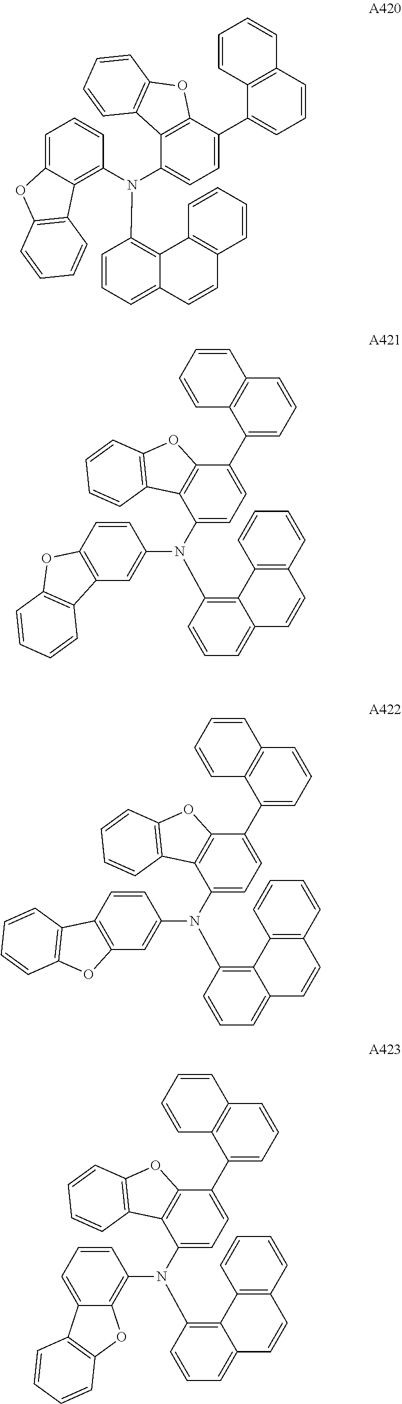

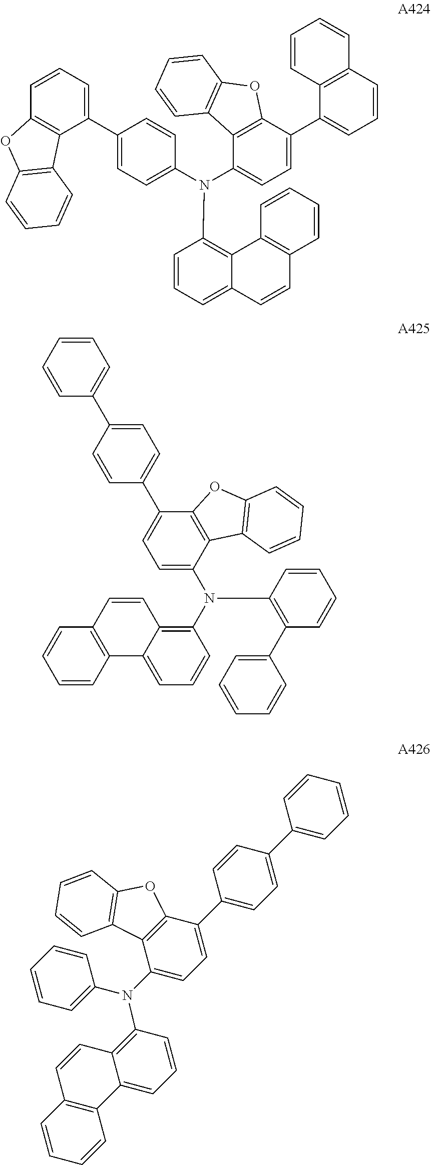 [SMILES: c1ccc2c(-c3ccc(N(c4ccc(-c5cccc6oc7ccccc7c56)cc4)c4cccc5ccc6ccccc6c45)c4c3oc3ccccc34)cccc2c1 ; c1ccc(-c2ccc(-c3ccc(N(c4ccccc4-c4ccccc4)c4cccc5c4ccc4ccccc45)c4c3oc3ccccc34)cc2)cc1 ; c1ccc(-c2ccc(-c3ccc(N(c4ccccc4)c4cccc5c4ccc4ccccc45)c4c3oc3ccccc34)cc2)cc1]

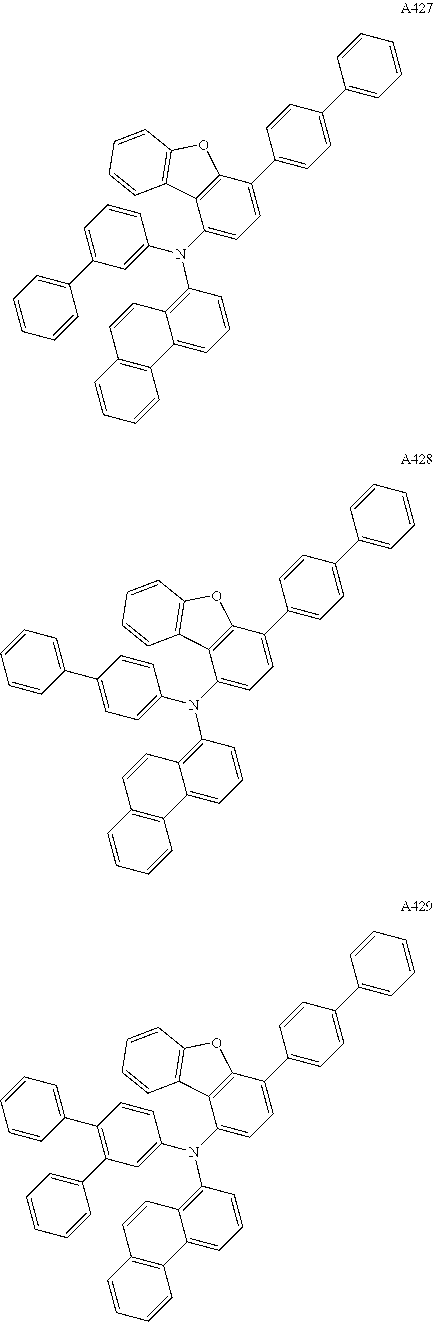 [SMILES: c1ccc(-c2ccc(-c3ccc(N(c4cccc(-c5ccccc5)c4)c4cccc5c4ccc4ccccc45)c4c3oc3ccccc34)cc2)cc1 ; c1ccc(-c2ccc(-c3ccc(N(c4ccc(-c5ccccc5)cc4)c4cccc5c4ccc4ccccc45)c4c3oc3ccccc34)cc2)cc1 ; c1ccc(-c2ccc(-c3ccc(N(c4ccc(-c5ccccc5)c(-c5ccccc5)c4)c4cccc5c4ccc4ccccc45)c4c3oc3ccccc34)cc2)cc1]

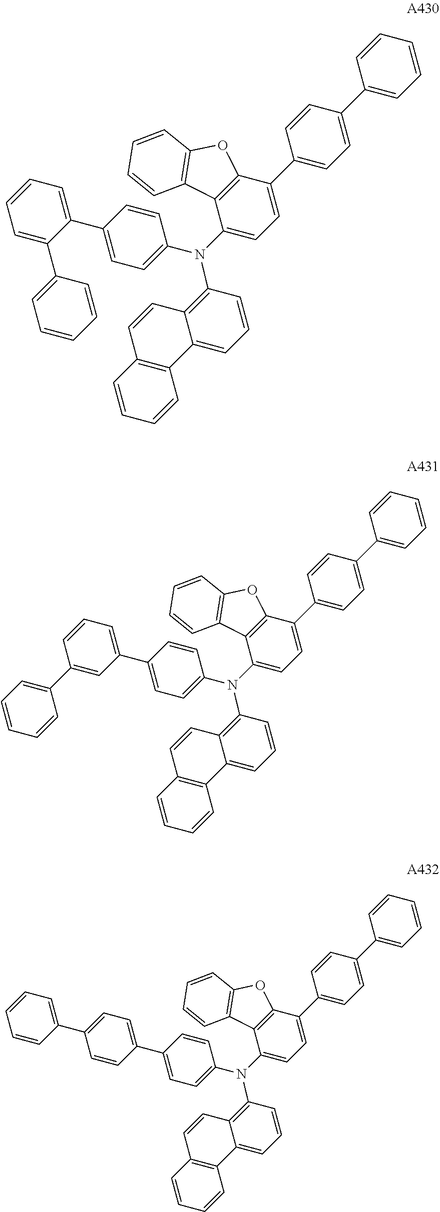 [SMILES: c1ccc(-c2ccc(-c3ccc(N(c4ccc(-c5ccccc5-c5ccccc5)cc4)c4cccc5c4ccc4ccccc45)c4c3oc3ccccc34)cc2)cc1 ; c1ccc(-c2ccc(-c3ccc(N(c4ccc(-c5cccc(-c6ccccc6)c5)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccc(-c6ccccc6)cc5)c5oc6ccccc6c45)cc3)cc2)cc1]

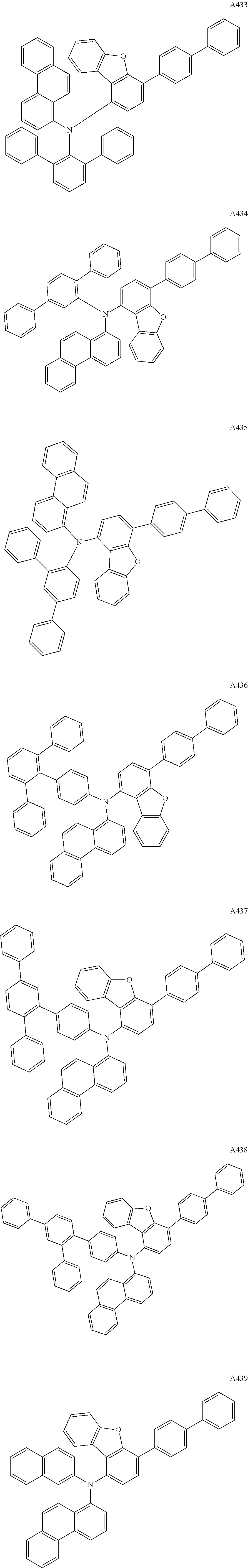

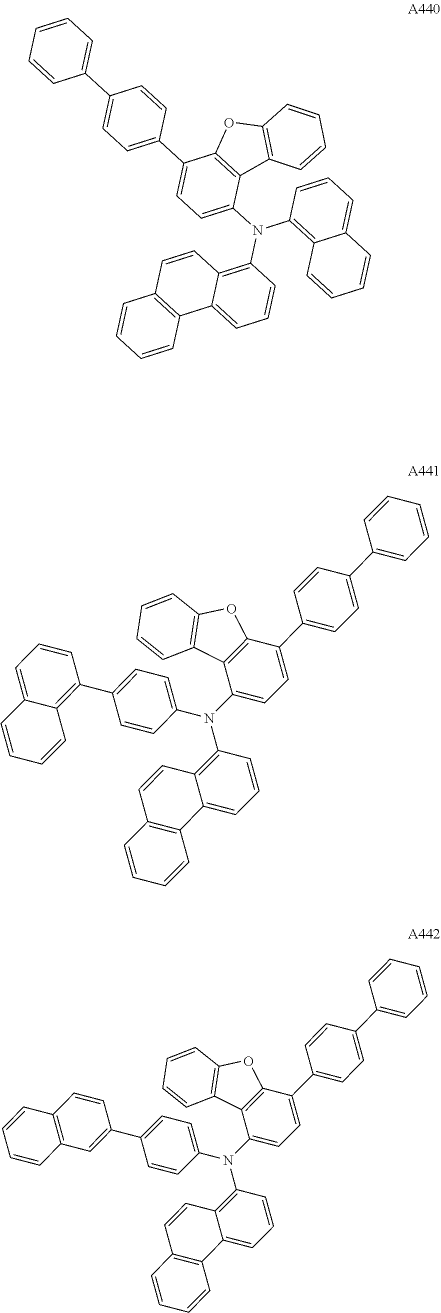 [SMILES: c1ccc(-c2ccc(-c3ccc(N(c4cccc5ccccc45)c4cccc5c4ccc4ccccc45)c4c3oc3ccccc34)cc2)cc1 ; c1ccc(-c2ccc(-c3ccc(N(c4ccc(-c5cccc6ccccc56)cc4)c4cccc5c4ccc4ccccc45)c4c3oc3ccccc34)cc2)cc1 ; c1ccc(-c2ccc(-c3ccc(N(c4ccc(-c5ccc6ccccc6c5)cc4)c4cccc5c4ccc4ccccc45)c4c3oc3ccccc34)cc2)cc1]

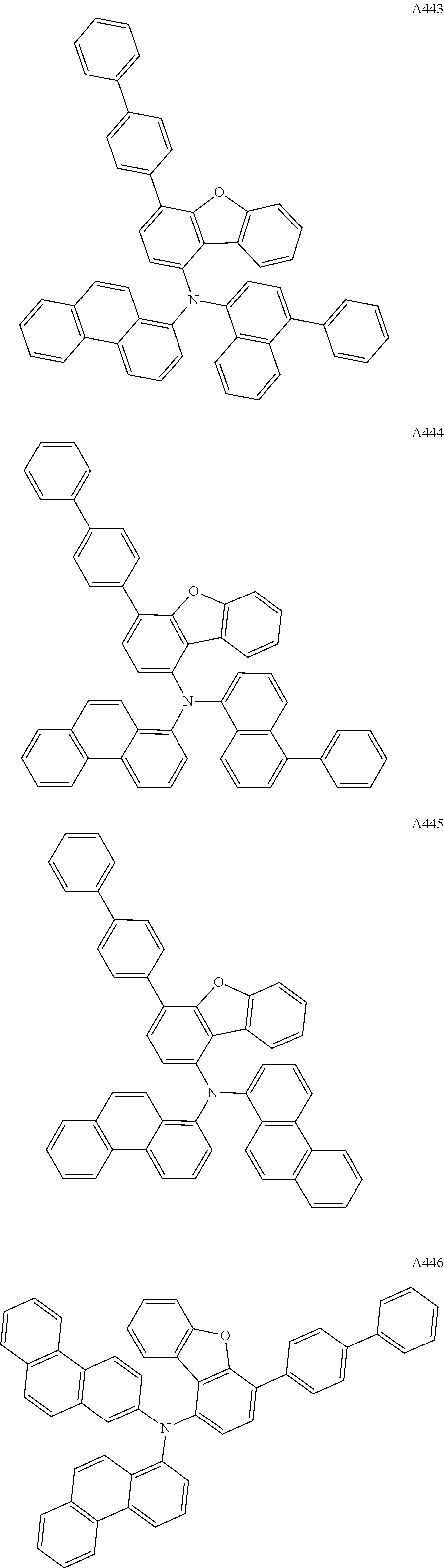 [SMILES: c1ccc(-c2ccc(-c3ccc(N(c4ccc(-c5ccccc5)c5ccccc45)c4cccc5c4ccc4ccccc45)c4c3oc3ccccc34)cc2)cc1 ; c1ccc(-c2ccc(-c3ccc(N(c4cccc5c(-c6ccccc6)cccc45)c4cccc5c4ccc4ccccc45)c4c3oc3ccccc34)cc2)cc1 ; c1ccc(-c2ccc(-c3ccc(N(c4cccc5c4ccc4ccccc45)c4cccc5c4ccc4ccccc45)c4c3oc3ccccc34)cc2)cc1 ; c1ccc(-c2ccc(-c3ccc(N(c4ccc5c(ccc6ccccc65)c4)c4cccc5c4ccc4ccccc45)c4c3oc3ccccc34)cc2)cc1]

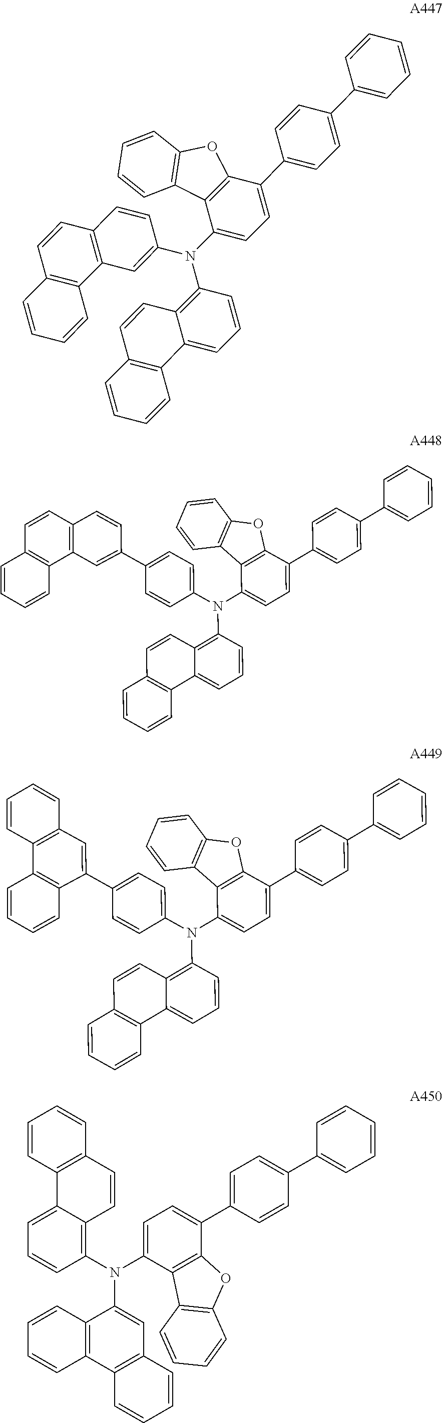 [SMILES: c1ccc(-c2ccc(-c3ccc(N(c4ccc5ccc6ccccc6c5c4)c4cccc5c4ccc4ccccc45)c4c3oc3ccccc34)cc2)cc1 ; c1ccc(-c2ccc(-c3ccc(N(c4ccc(-c5ccc6ccc7ccccc7c6c5)cc4)c4cccc5c4ccc4ccccc45)c4c3oc3ccccc34)cc2)cc1 ; c1ccc(-c2ccc(-c3ccc(N(c4ccc(-c5cc6ccccc6c6ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4cc5ccccc5c5ccccc45)c4c3oc3ccccc34)cc2)cc1]

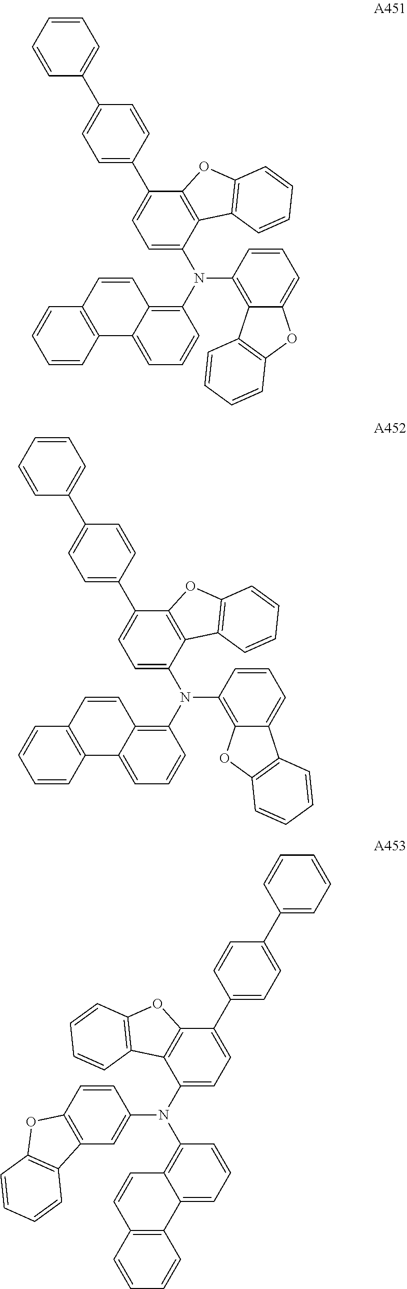

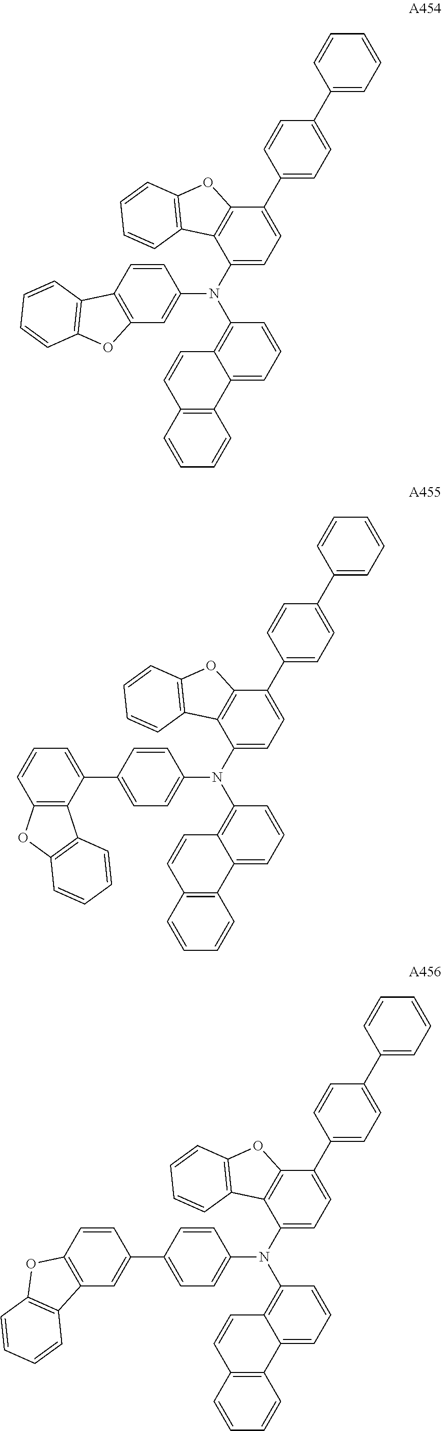 [SMILES: c1ccc(-c2ccc(-c3ccc(N(c4ccc5c(c4)oc4ccccc45)c4cccc5c4ccc4ccccc45)c4c3oc3ccccc34)cc2)cc1 ; c1ccc(-c2ccc(-c3ccc(N(c4ccc(-c5cccc6oc7ccccc7c56)cc4)c4cccc5c4ccc4ccccc45)c4c3oc3ccccc34)cc2)cc1 ; c1ccc(-c2ccc(-c3ccc(N(c4ccc(-c5ccc6oc7ccccc7c6c5)cc4)c4cccc5c4ccc4ccccc45)c4c3oc3ccccc34)cc2)cc1]

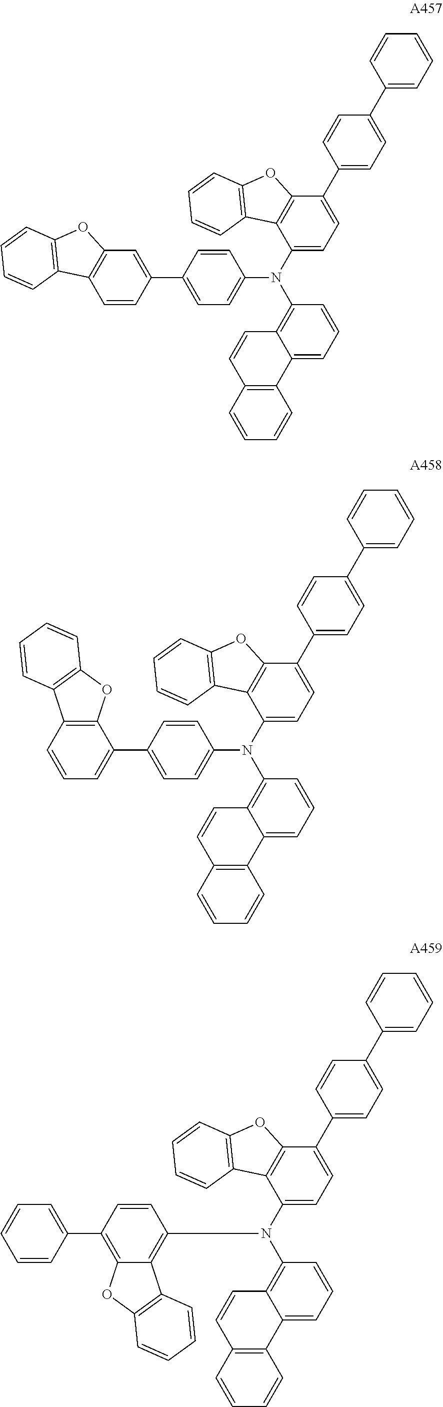 [SMILES: c1ccc(-c2ccc(-c3ccc(N(c4ccc(-c5ccc6c(c5)oc5ccccc56)cc4)c4cccc5c4ccc4ccccc45)c4c3oc3ccccc34)cc2)cc1 ; c1ccc(-c2ccc(-c3ccc(N(c4ccc(-c5cccc6c5oc5ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccccc5)c5oc6ccccc6c45)c4c3oc3ccccc34)cc2)cc1]

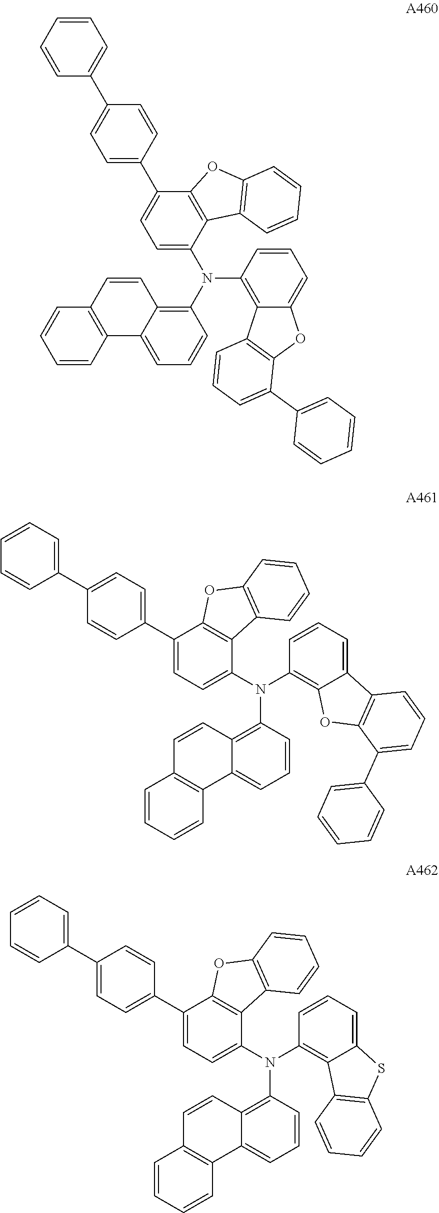 [SMILES: c1ccc(-c2ccc(-c3ccc(N(c4cccc5c4ccc4ccccc45)c4cccc5oc6c(-c7ccccc7)cccc6c45)c4c3oc3ccccc34)cc2)cc1 ; c1ccc(-c2ccc(-c3ccc(N(c4cccc5c4ccc4ccccc45)c4cccc5c4oc4c(-c6ccccc6)cccc45)c4c3oc3ccccc34)cc2)cc1 ; c1ccc(-c2ccc(-c3ccc(N(c4cccc5c4ccc4ccccc45)c4cccc5sc6ccccc6c45)c4c3oc3ccccc34)cc2)cc1]

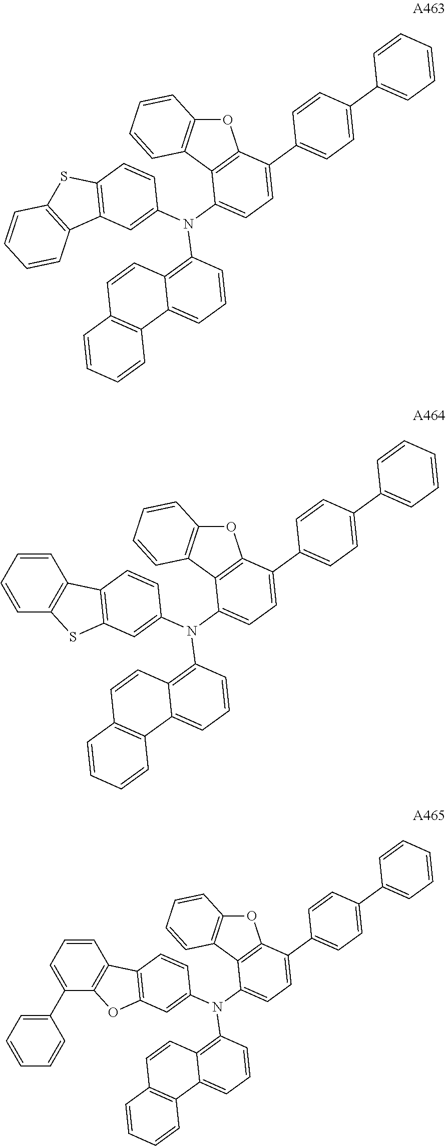 [SMILES: c1ccc(-c2ccc(-c3ccc(N(c4ccc5sc6ccccc6c5c4)c4cccc5c4ccc4ccccc45)c4c3oc3ccccc34)cc2)cc1 ; c1ccc(-c2ccc(-c3ccc(N(c4ccc5c(c4)sc4ccccc45)c4cccc5c4ccc4ccccc45)c4c3oc3ccccc34)cc2)cc1 ; c1ccc(-c2ccc(-c3ccc(N(c4ccc5c(c4)oc4c(-c6ccccc6)cccc45)c4cccc5c4ccc4ccccc45)c4c3oc3ccccc34)cc2)cc1]

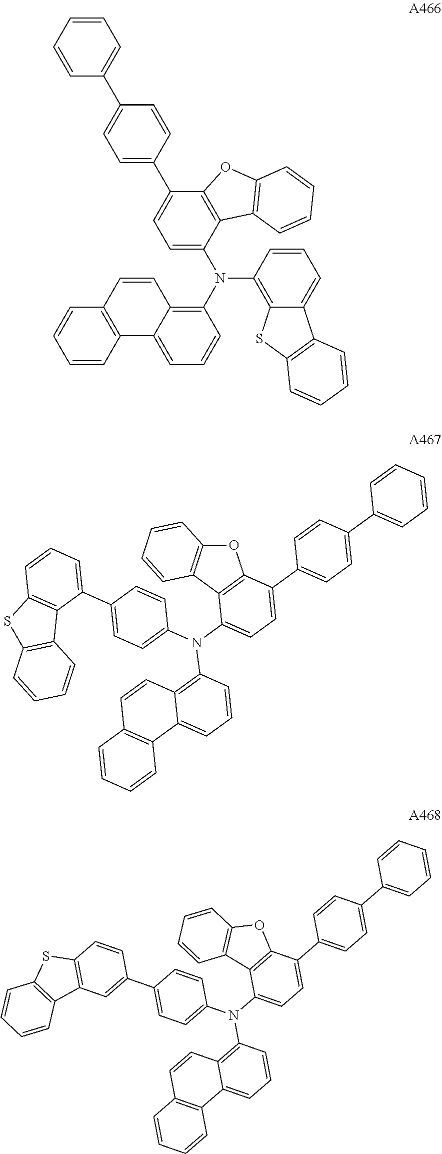 [SMILES: c1ccc(-c2ccc(-c3ccc(N(c4cccc5c4ccc4ccccc45)c4cccc5c4sc4ccccc45)c4c3oc3ccccc34)cc2)cc1 ; c1ccc(-c2ccc(-c3ccc(N(c4ccc(-c5cccc6sc7ccccc7c56)cc4)c4cccc5c4ccc4ccccc45)c4c3oc3ccccc34)cc2)cc1 ; c1ccc(-c2ccc(-c3ccc(N(c4ccc(-c5ccc6sc7ccccc7c6c5)cc4)c4cccc5c4ccc4ccccc45)c4c3oc3ccccc34)cc2)cc1]

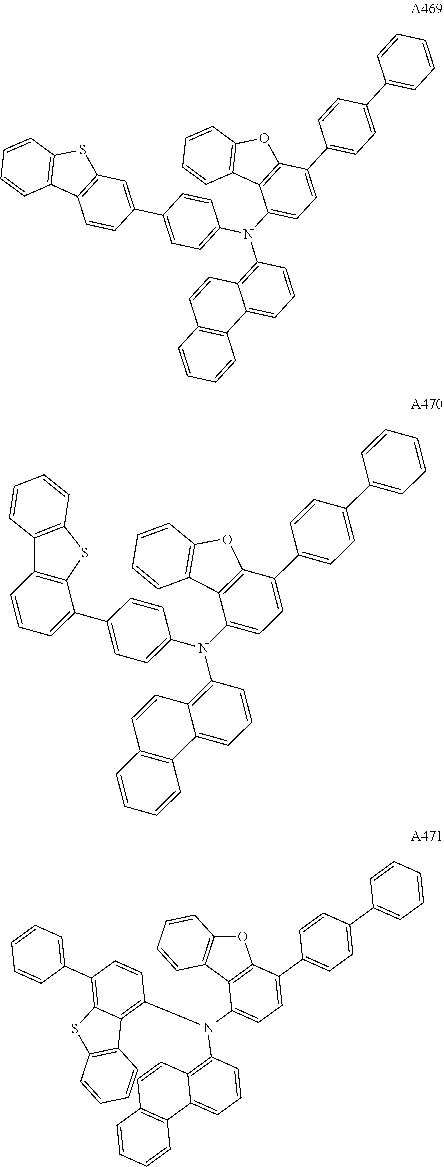 [SMILES: c1ccc(-c2ccc(-c3ccc(N(c4ccc(-c5ccc6c(c5)sc5ccccc56)cc4)c4cccc5c4ccc4ccccc45)c4c3oc3ccccc34)cc2)cc1 ; c1ccc(-c2ccc(-c3ccc(N(c4ccc(-c5cccc6c5sc5ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccccc5)c5sc6ccccc6c45)c4c3oc3ccccc34)cc2)cc1]

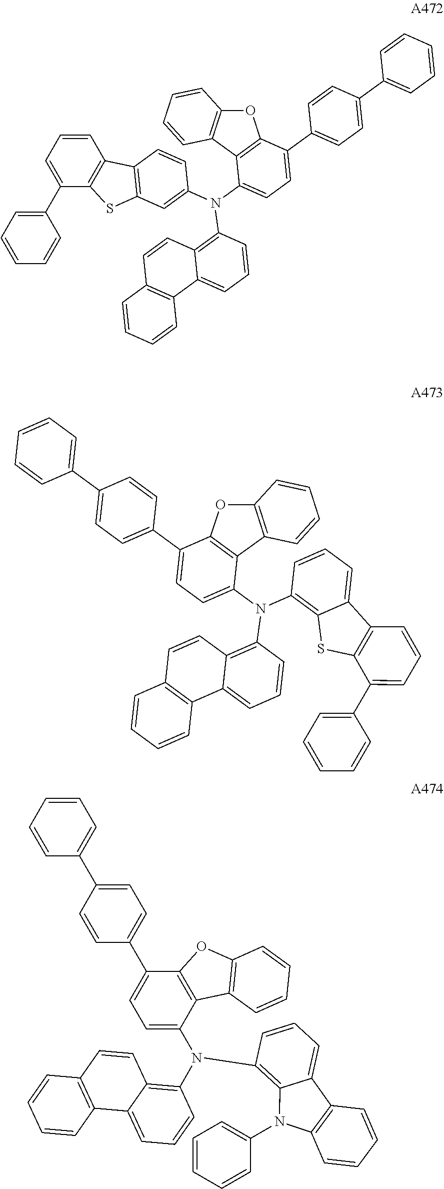 [SMILES: c1ccc(-c2ccc(-c3ccc(N(c4ccc5c(c4)sc4c(-c6ccccc6)cccc45)c4cccc5c4ccc4ccccc45)c4c3oc3ccccc34)cc2)cc1 ; c1ccc(-c2ccc(-c3ccc(N(c4cccc5c4ccc4ccccc45)c4cccc5c4sc4c(-c6ccccc6)cccc45)c4c3oc3ccccc34)cc2)cc1 ; c1ccc(-c2ccc(-c3ccc(N(c4cccc5c4ccc4ccccc45)c4cccc5c6ccccc6n(-c6ccccc6)c45)c4c3oc3ccccc34)cc2)cc1]

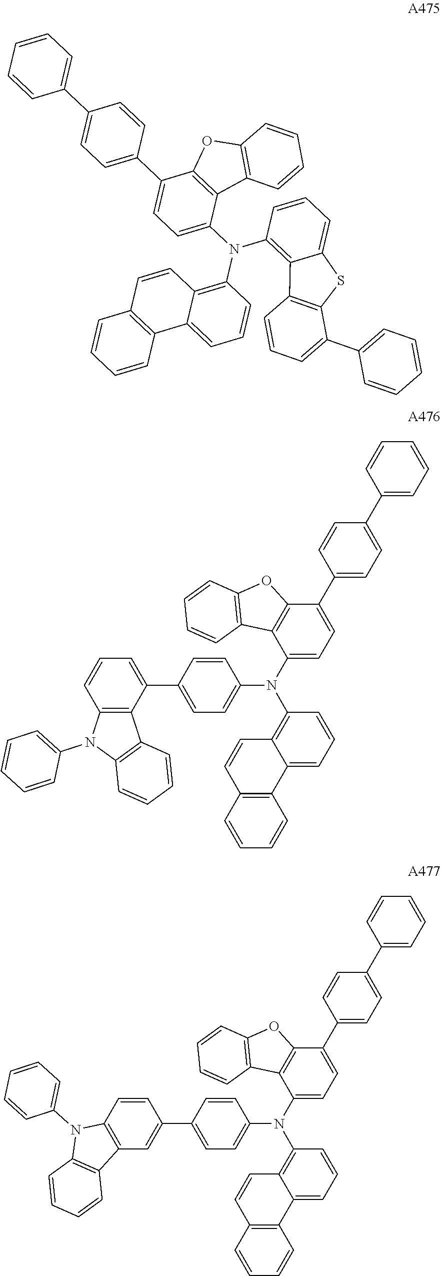 [SMILES: c1ccc(-c2ccc(-c3ccc(N(c4cccc5c4ccc4ccccc45)c4cccc5sc6c(-c7ccccc7)cccc6c45)c4c3oc3ccccc34)cc2)cc1 ; c1ccc(-c2ccc(-c3ccc(N(c4ccc(-c5cccc6c5c5ccccc5n6-c5ccccc5)cc4)c4cccc5c4ccc4ccccc45)c4c3oc3ccccc34)cc2)cc1 ; c1ccc(-c2ccc(-c3ccc(N(c4ccc(-c5ccc6c(c5)c5ccccc5n6-c5ccccc5)cc4)c4cccc5c4ccc4ccccc45)c4c3oc3ccccc34)cc2)cc1]

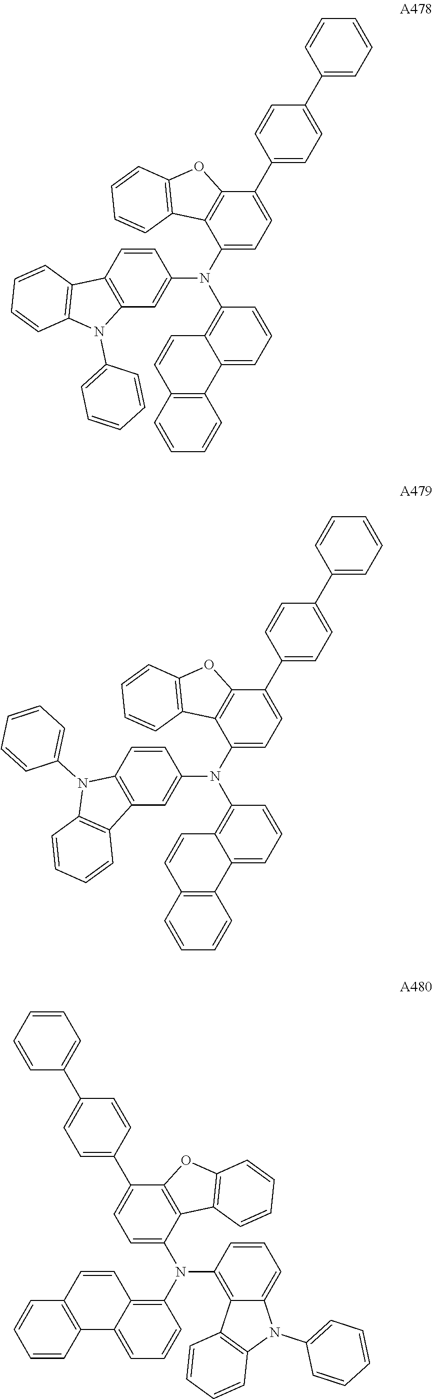 [SMILES: c1ccc(-c2ccc(-c3ccc(N(c4ccc5c6ccccc6n(-c6ccccc6)c5c4)c4cccc5c4ccc4ccccc45)c4c3oc3ccccc34)cc2)cc1 ; c1ccc(-c2ccc(-c3ccc(N(c4ccc5c(c4)c4ccccc4n5-c4ccccc4)c4cccc5c4ccc4ccccc45)c4c3oc3ccccc34)cc2)cc1 ; c1ccc(-c2ccc(-c3ccc(N(c4cccc5c4ccc4ccccc45)c4cccc5c4c4ccccc4n5-c4ccccc4)c4c3oc3ccccc34)cc2)cc1]

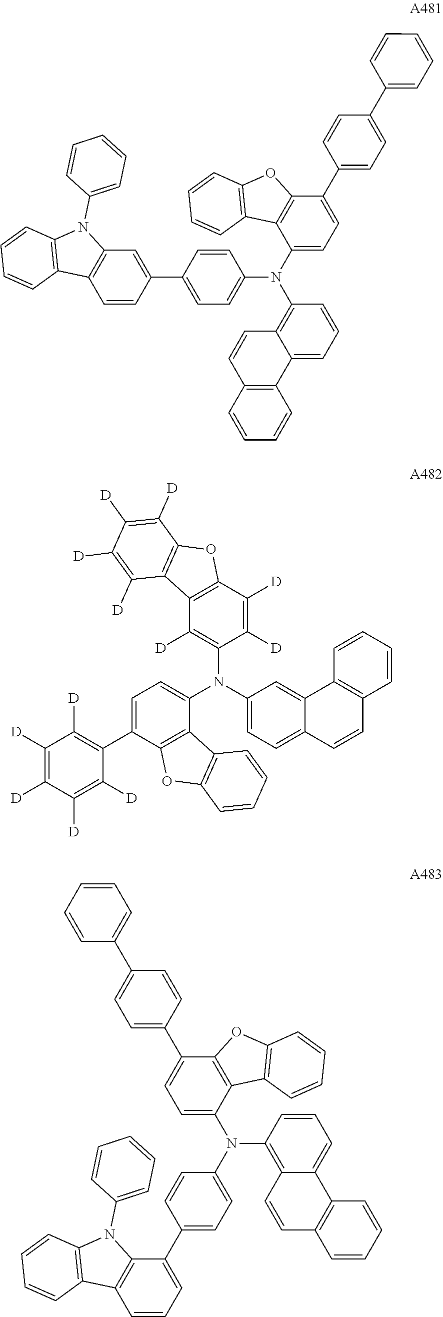 [SMILES: c1ccc(-c2ccc(-c3ccc(N(c4ccc(-c5ccc6c7ccccc7n(-c7ccccc7)c6c5)cc4)c4cccc5c4ccc4ccccc45)c4c3oc3ccccc34)cc2)cc1 ; [2H]c1c([2H])c([2H])c(-c2ccc(N(c3ccc4ccc5ccccc5c4c3)c3c([2H])c([2H])c4oc5c([2H])c([2H])c([2H])c([2H])c5c4c3[2H])c3c2oc2ccccc23)c([2H])c1[2H] ; c1ccc(-c2ccc(-c3ccc(N(c4ccc(-c5cccc6c7ccccc7n(-c7ccccc7)c56)cc4)c4cccc5c4ccc4ccccc45)c4c3oc3ccccc34)cc2)cc1]

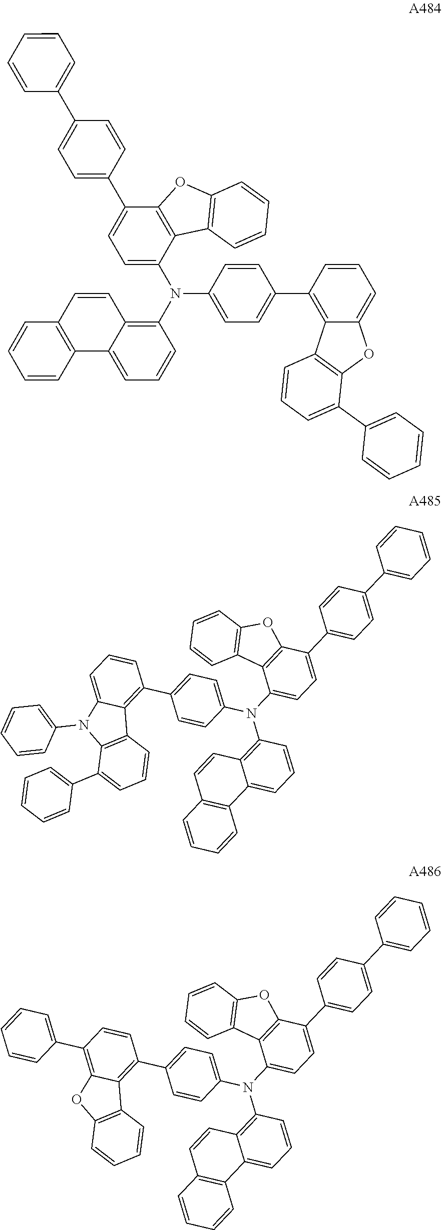 [SMILES: c1ccc(-c2ccc(-c3ccc(N(c4ccc(-c5cccc6oc7c(-c8ccccc8)cccc7c56)cc4)c4cccc5c4ccc4ccccc45)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4cccc5c4ccc4ccccc45)c4c3oc3ccccc34)cc2)cc1 ; c1ccc(-c2ccc(-c3ccc(N(c4ccc(-c5ccc(-c6ccccc6)c6oc7ccccc7c56)cc4)c4cccc5c4ccc4ccccc45)c4c3oc3ccccc34)cc2)cc1]

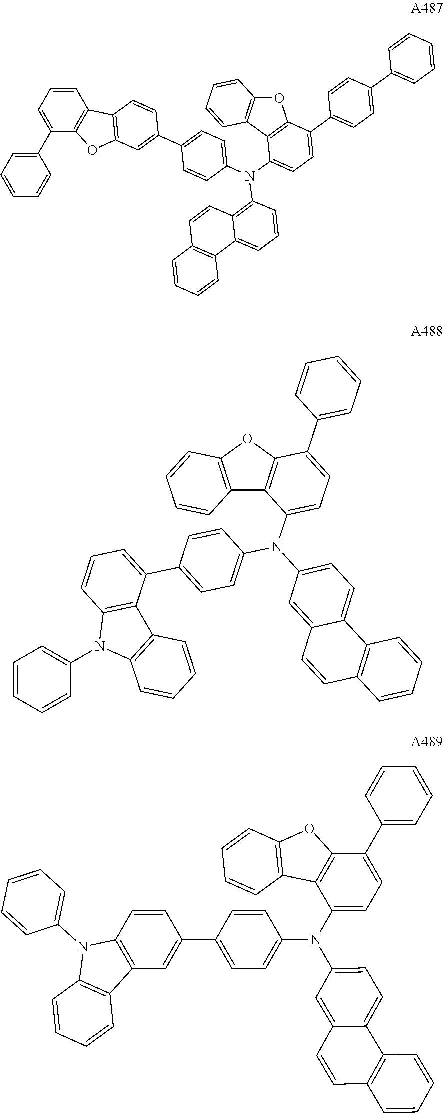 [SMILES: c1ccc(-c2ccc(-c3ccc(N(c4ccc(-c5ccc6c(c5)oc5c(-c7ccccc7)cccc56)cc4)c4cccc5c4ccc4ccccc45)c4c3oc3ccccc34)cc2)cc1 ; c1ccc(-c2ccc(N(c3ccc(-c4cccc5c4c4ccccc4n5-c4ccccc4)cc3)c3ccc4c(ccc5ccccc54)c3)c3c2oc2ccccc23)cc1 ; c1ccc(-c2ccc(N(c3ccc(-c4ccc5c(c4)c4ccccc4n5-c4ccccc4)cc3)c3ccc4c(ccc5ccccc54)c3)c3c2oc2ccccc23)cc1]

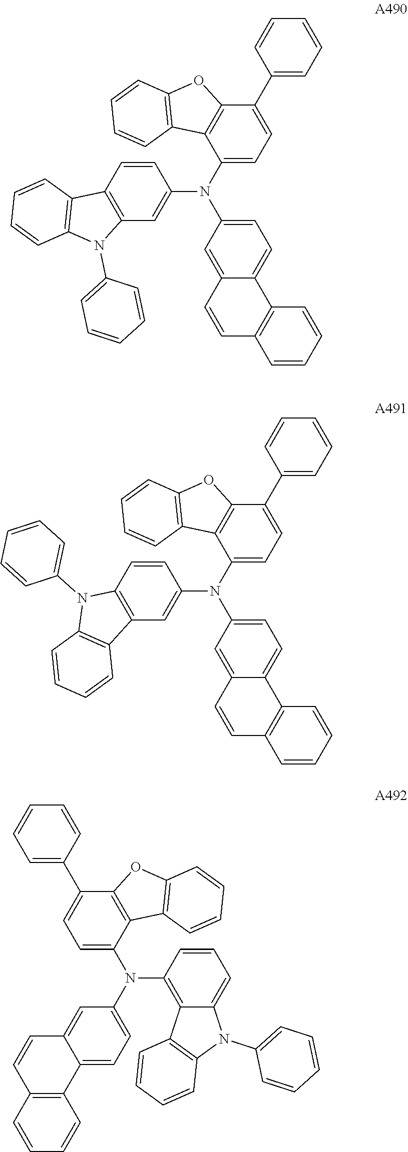 [SMILES: c1ccc(-c2ccc(N(c3ccc4c(ccc5ccccc54)c3)c3ccc4c5ccccc5n(-c5ccccc5)c4c3)c3c2oc2ccccc23)cc1 ; c1ccc(-c2ccc(N(c3ccc4c(ccc5ccccc54)c3)c3ccc4c(c3)c3ccccc3n4-c3ccccc3)c3c2oc2ccccc23)cc1 ; c1ccc(-c2ccc(N(c3ccc4c(ccc5ccccc54)c3)c3cccc4c3c3ccccc3n4-c3ccccc3)c3c2oc2ccccc23)cc1]

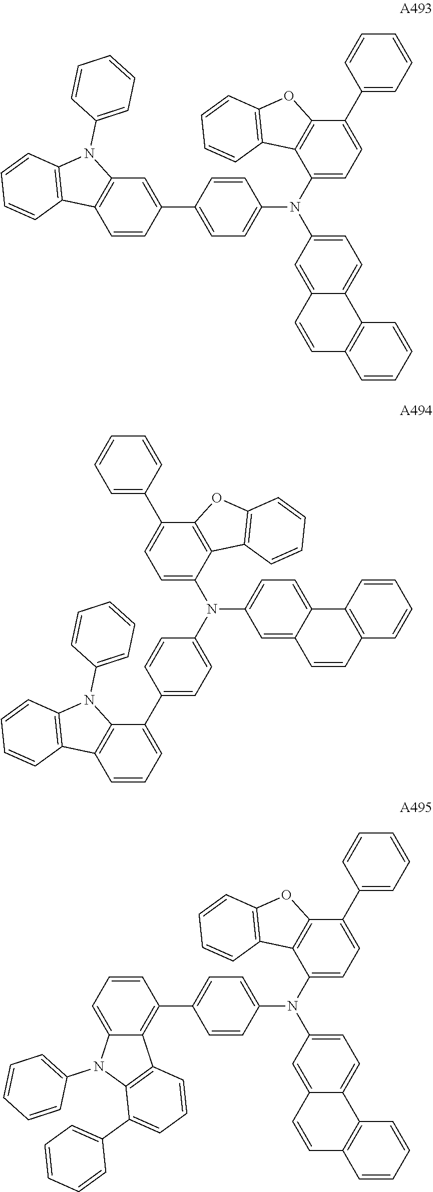 [SMILES: c1ccc(-c2ccc(N(c3ccc(-c4ccc5c6ccccc6n(-c6ccccc6)c5c4)cc3)c3ccc4c(ccc5ccccc54)c3)c3c2oc2ccccc23)cc1 ; c1ccc(-c2ccc(N(c3ccc(-c4cccc5c6ccccc6n(-c6ccccc6)c45)cc3)c3ccc4c(ccc5ccccc54)c3)c3c2oc2ccccc23)cc1 ; c1ccc(-c2ccc(N(c3ccc(-c4cccc5c4c4cccc(-c6ccccc6)c4n5-c4ccccc4)cc3)c3ccc4c(ccc5ccccc54)c3)c3c2oc2ccccc23)cc1]

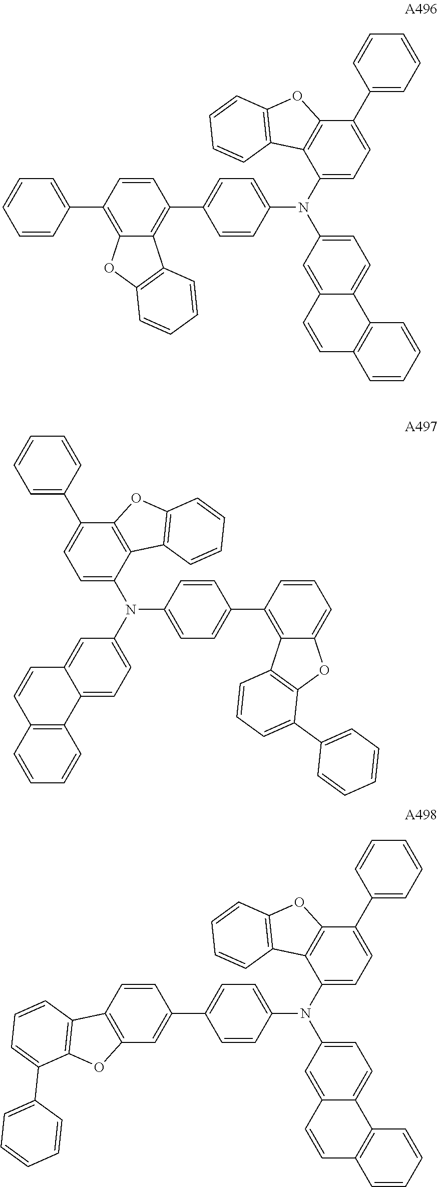 [SMILES: c1ccc(-c2ccc(-c3ccc(N(c4ccc5c(ccc6ccccc65)c4)c4ccc(-c5ccccc5)c5oc6ccccc6c45)cc3)c3c2oc2ccccc23)cc1 ; c1ccc(-c2cccc3c2oc2cccc(-c4ccc(N(c5ccc6c(ccc7ccccc76)c5)c5ccc(-c6ccccc6)c6oc7ccccc7c56)cc4)c23)cc1 ; c1ccc(-c2cccc3c2oc2cc(-c4ccc(N(c5ccc6c(ccc7ccccc76)c5)c5ccc(-c6ccccc6)c6oc7ccccc7c56)cc4)ccc23)cc1]

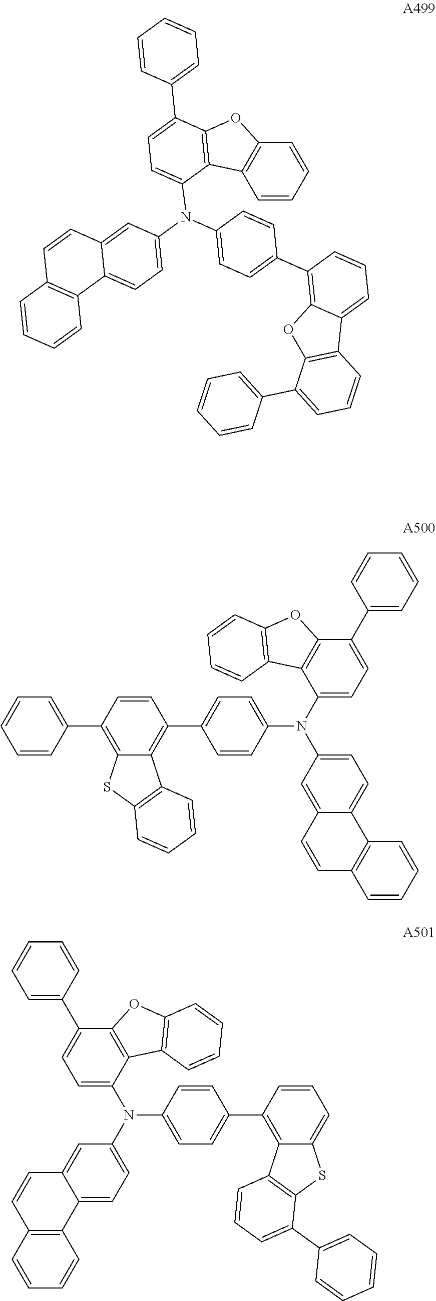 [SMILES: c1ccc(-c2cccc3c2oc2c(-c4ccc(N(c5ccc6c(ccc7ccccc76)c5)c5ccc(-c6ccccc6)c6oc7ccccc7c56)cc4)cccc23)cc1 ; c1ccc(-c2ccc(N(c3ccc(-c4ccc(-c5ccccc5)c5sc6ccccc6c45)cc3)c3ccc4c(ccc5ccccc54)c3)c3c2oc2ccccc23)cc1 ; c1ccc(-c2ccc(N(c3ccc(-c4cccc5sc6c(-c7ccccc7)cccc6c45)cc3)c3ccc4c(ccc5ccccc54)c3)c3c2oc2ccccc23)cc1]

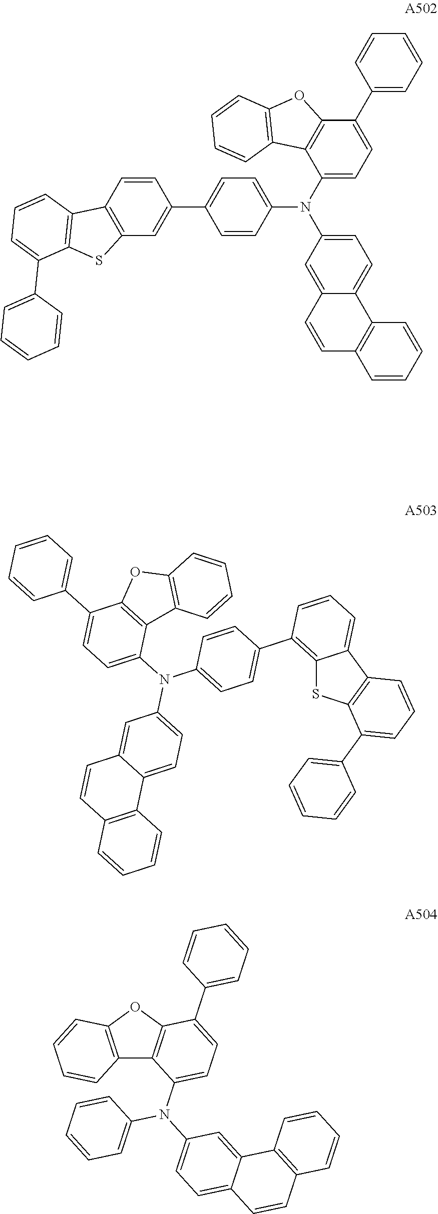 [SMILES: c1ccc(-c2ccc(N(c3ccc(-c4ccc5c(c4)sc4c(-c6ccccc6)cccc45)cc3)c3ccc4c(ccc5ccccc54)c3)c3c2oc2ccccc23)cc1 ; c1ccc(-c2ccc(N(c3ccc(-c4cccc5c4sc4c(-c6ccccc6)cccc45)cc3)c3ccc4c(ccc5ccccc54)c3)c3c2oc2ccccc23)cc1 ; c1ccc(-c2ccc(N(c3ccccc3)c3ccc4ccc5ccccc5c4c3)c3c2oc2ccccc23)cc1]

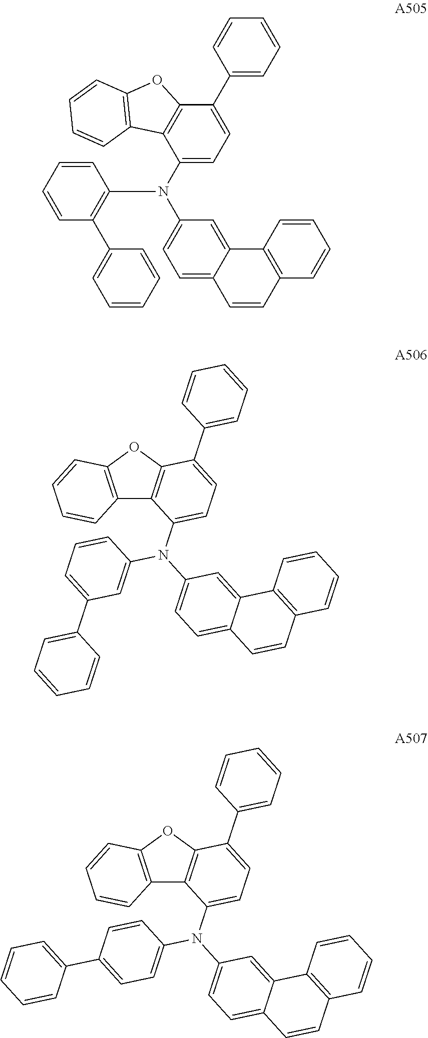 [SMILES: c1ccc(-c2ccccc2N(c2ccc3ccc4ccccc4c3c2)c2ccc(-c3ccccc3)c3oc4ccccc4c23)cc1 ; c1ccc(-c2cccc(N(c3ccc4ccc5ccccc5c4c3)c3ccc(-c4ccccc4)c4oc5ccccc5c34)c2)cc1 ; c1ccc(-c2ccc(N(c3ccc4ccc5ccccc5c4c3)c3ccc(-c4ccccc4)c4oc5ccccc5c34)cc2)cc1]

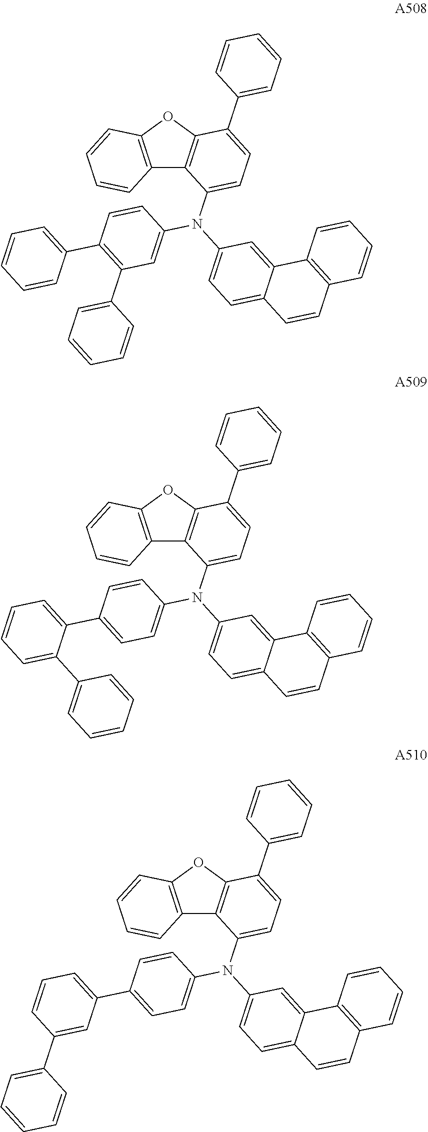 [SMILES: c1ccc(-c2ccc(N(c3ccc4ccc5ccccc5c4c3)c3ccc(-c4ccccc4)c4oc5ccccc5c34)cc2-c2ccccc2)cc1 ; c1ccc(-c2ccccc2-c2ccc(N(c3ccc4ccc5ccccc5c4c3)c3ccc(-c4ccccc4)c4oc5ccccc5c34)cc2)cc1 ; c1ccc(-c2cccc(-c3ccc(N(c4ccc5ccc6ccccc6c5c4)c4ccc(-c5ccccc5)c5oc6ccccc6c45)cc3)c2)cc1]

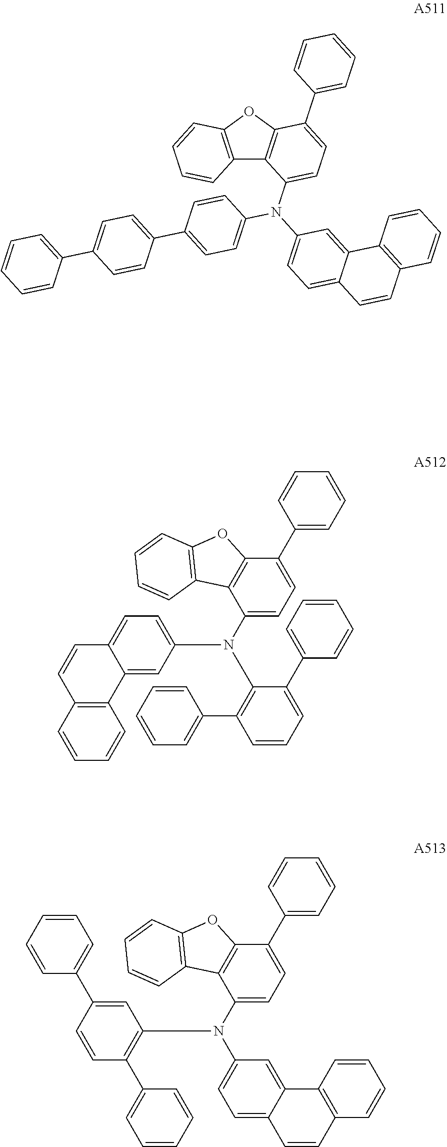 [SMILES: c1ccc(-c2ccc(-c3ccc(N(c4ccc5ccc6ccccc6c5c4)c4ccc(-c5ccccc5)c5oc6ccccc6c45)cc3)cc2)cc1 ; c1ccc(-c2cccc(-c3ccccc3)c2N(c2ccc3ccc4ccccc4c3c2)c2ccc(-c3ccccc3)c3oc4ccccc4c23)cc1 ; c1ccc(-c2ccc(-c3ccccc3)c(N(c3ccc4ccc5ccccc5c4c3)c3ccc(-c4ccccc4)c4oc5ccccc5c34)c2)cc1]

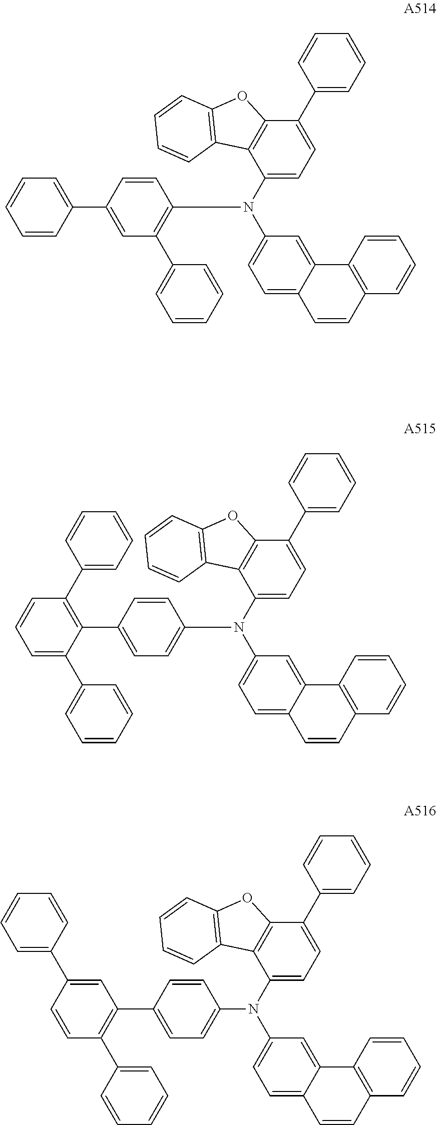 [SMILES: c1ccc(-c2ccc(N(c3ccc4ccc5ccccc5c4c3)c3ccc(-c4ccccc4)c4oc5ccccc5c34)c(-c3ccccc3)c2)cc1 ; c1ccc(-c2cccc(-c3ccccc3)c2-c2ccc(N(c3ccc4ccc5ccccc5c4c3)c3ccc(-c4ccccc4)c4oc5ccccc5c34)cc2)cc1 ; c1ccc(-c2ccc(-c3ccccc3)c(-c3ccc(N(c4ccc5ccc6ccccc6c5c4)c4ccc(-c5ccccc5)c5oc6ccccc6c45)cc3)c2)cc1]

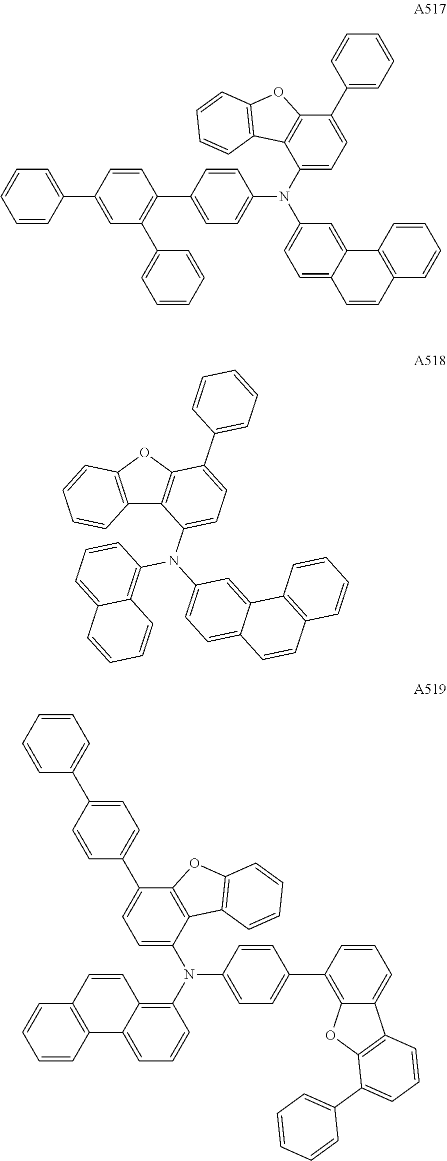 [SMILES: c1ccc(-c2ccc(-c3ccc(N(c4ccc5ccc6ccccc6c5c4)c4ccc(-c5ccccc5)c5oc6ccccc6c45)cc3)c(-c3ccccc3)c2)cc1 ; c1ccc(-c2ccc(N(c3ccc4ccc5ccccc5c4c3)c3cccc4ccccc34)c3c2oc2ccccc23)cc1 ; c1ccc(-c2ccc(-c3ccc(N(c4ccc(-c5cccc6c5oc5c(-c7ccccc7)cccc56)cc4)c4cccc5c4ccc4ccccc45)c4c3oc3ccccc34)cc2)cc1]

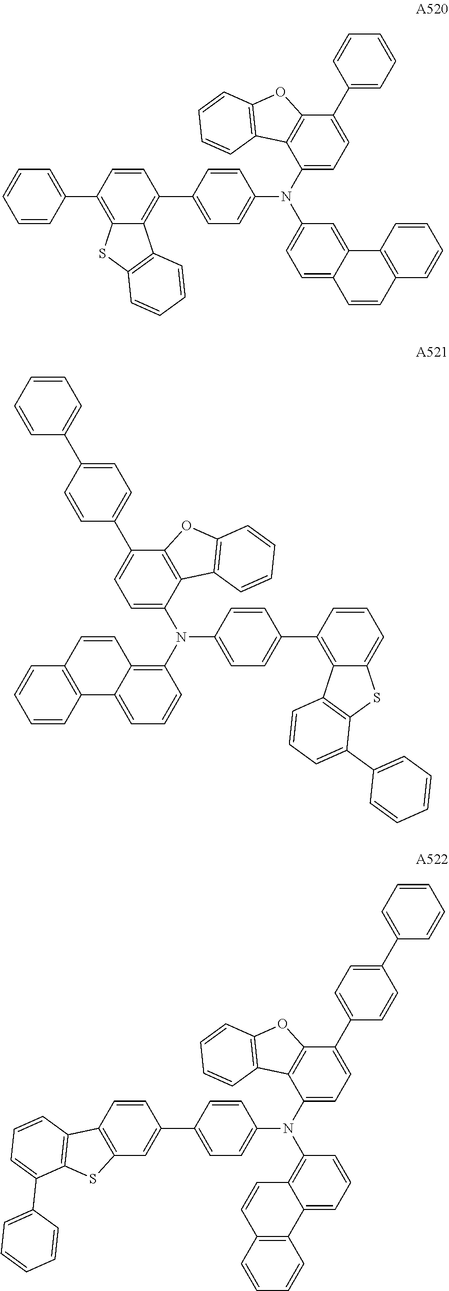 [SMILES: c1ccc(-c2ccc(N(c3ccc(-c4ccc(-c5ccccc5)c5sc6ccccc6c45)cc3)c3ccc4ccc5ccccc5c4c3)c3c2oc2ccccc23)cc1 ; c1ccc(-c2ccc(-c3ccc(N(c4ccc(-c5cccc6sc7c(-c8ccccc8)cccc7c56)cc4)c4cccc5c4ccc4ccccc45)c4c3oc3ccccc34)cc2)cc1 ; c1ccc(-c2ccc(-c3ccc(N(c4ccc(-c5ccc6c(c5)sc5c(-c7ccccc7)cccc56)cc4)c4cccc5c4ccc4ccccc45)c4c3oc3ccccc34)cc2)cc1]

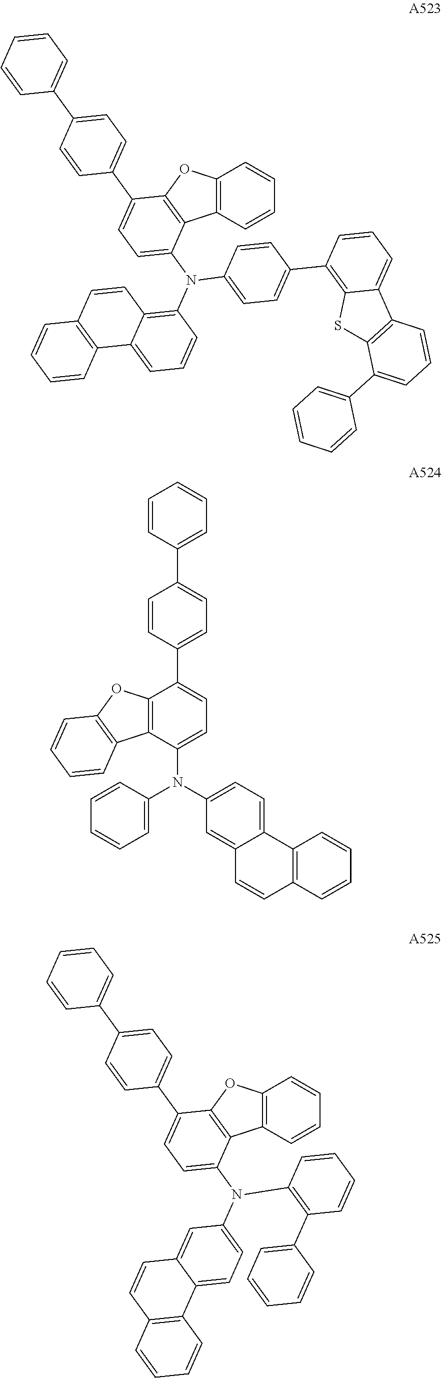 [SMILES: c1ccc(-c2ccc(-c3ccc(N(c4ccc(-c5cccc6c5sc5c(-c7ccccc7)cccc56)cc4)c4cccc5c4ccc4ccccc45)c4c3oc3ccccc34)cc2)cc1 ; c1ccc(-c2ccc(-c3ccc(N(c4ccccc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccccc4-c4ccccc4)c4c3oc3ccccc34)cc2)cc1]

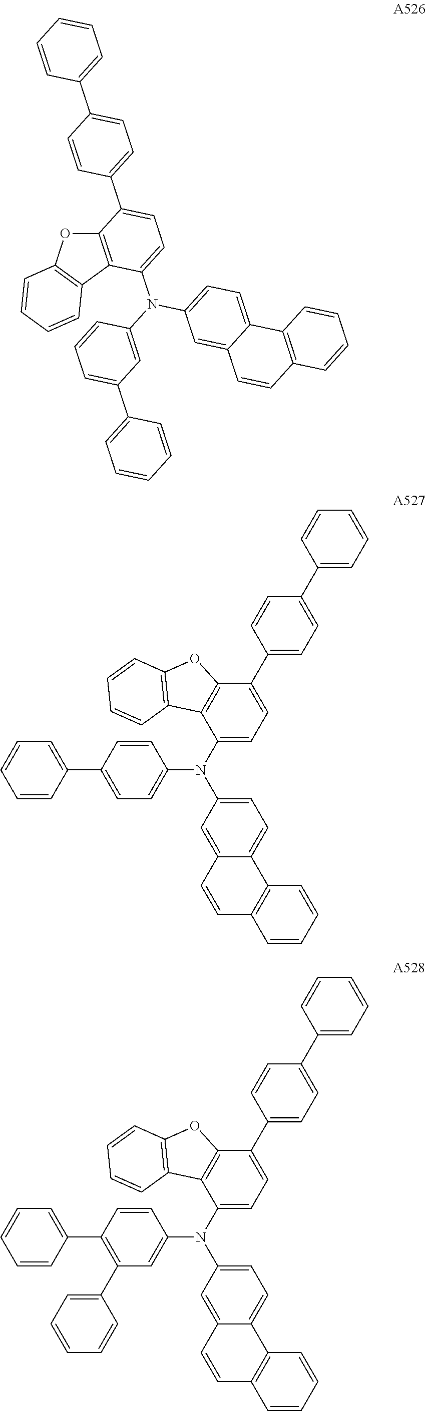

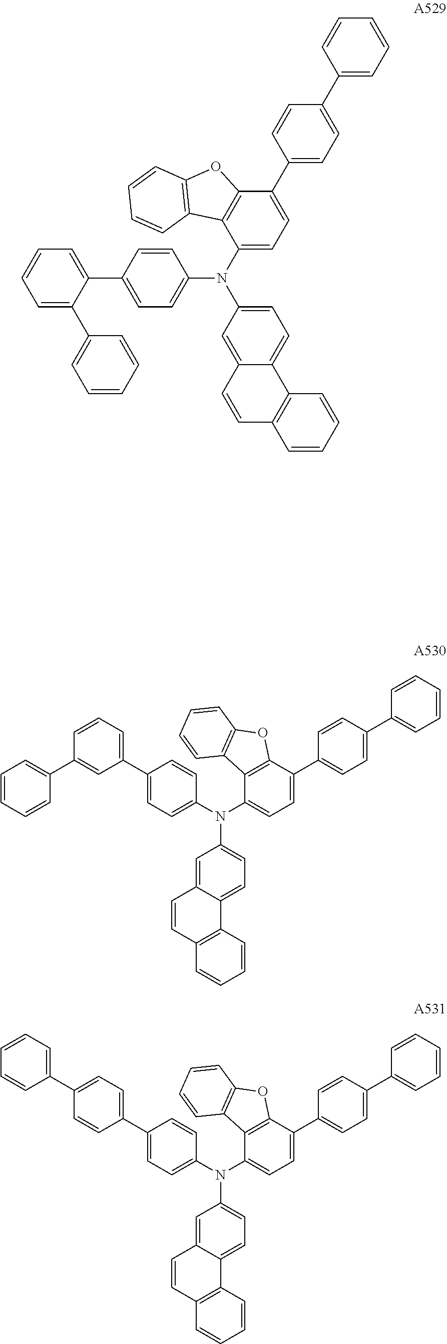 [SMILES: c1ccc(-c2ccc(-c3ccc(N(c4ccc(-c5ccccc5-c5ccccc5)cc4)c4ccc5c(ccc6ccccc65)c4)c4c3oc3ccccc34)cc2)cc1 ; c1ccc(-c2ccc(-c3ccc(N(c4ccc(-c5cccc(-c6ccccc6)c5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccc(-c6ccccc6)cc5)c5oc6ccccc6c45)cc3)cc2)cc1]

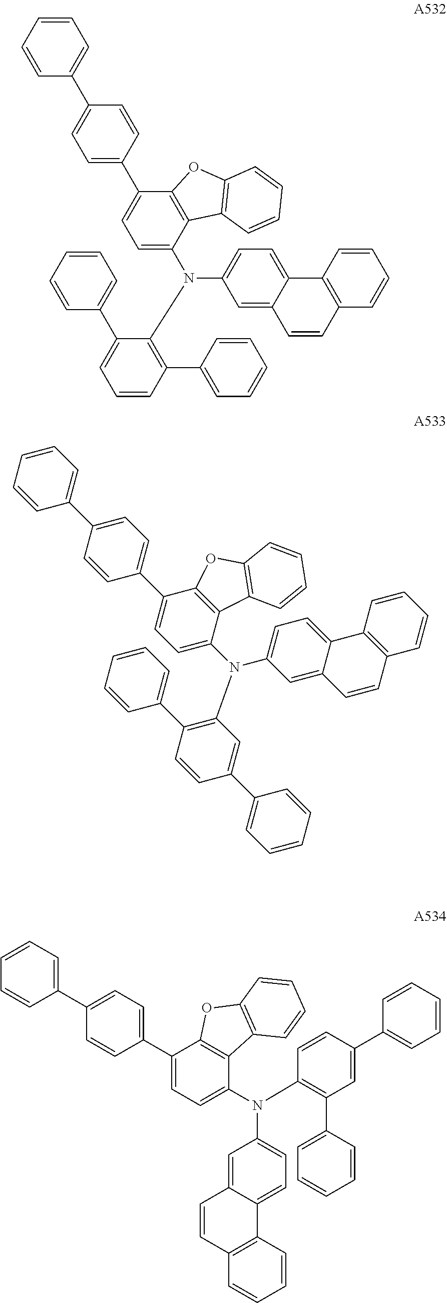 [SMILES: c1ccc(-c2ccc(-c3ccc(N(c4ccc5c(ccc6ccccc65)c4)c4c(-c5ccccc5)cccc4-c4ccccc4)c4c3oc3ccccc34)cc2)cc1 ; c1ccc(-c2ccc(-c3ccc(N(c4ccc5c(ccc6ccccc65)c4)c4cc(-c5ccccc5)ccc4-c4ccccc4)c4c3oc3ccccc34)cc2)cc1 ; c1ccc(-c2ccc(-c3ccc(N(c4ccc5c(ccc6ccccc65)c4)c4ccc(-c5ccccc5)cc4-c4ccccc4)c4c3oc3ccccc34)cc2)cc1]

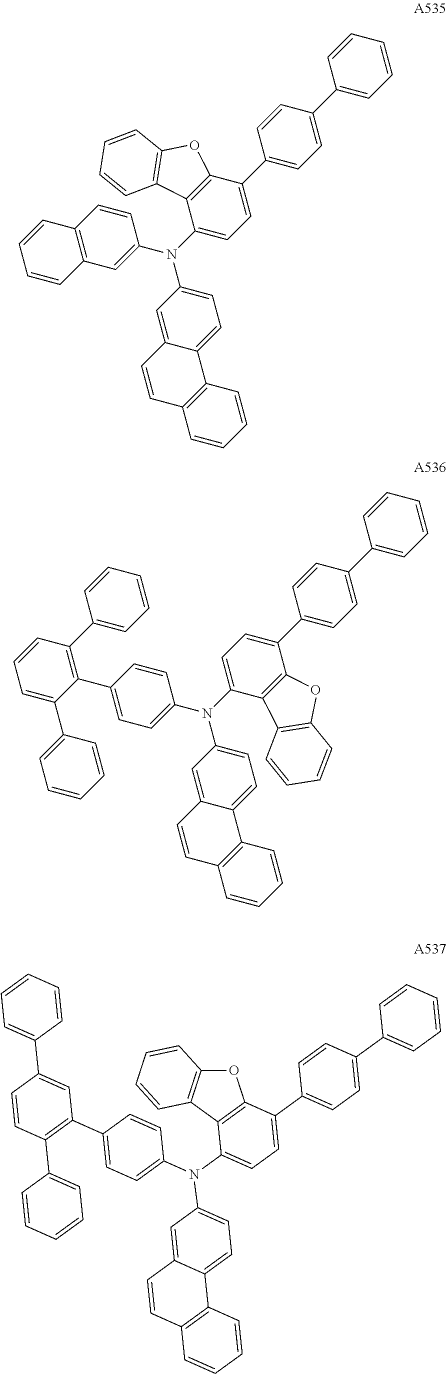 [SMILES: c1ccc(-c2ccc(-c3ccc(N(c4ccc5ccccc5c4)c4ccc5c(ccc6ccccc65)c4)c4c3oc3ccccc34)cc2)cc1 ; c1ccc(-c2ccc(-c3ccc(N(c4ccc(-c5c(-c6ccccc6)cccc5-c5ccccc5)cc4)c4ccc5c(ccc6ccccc65)c4)c4c3oc3ccccc34)cc2)cc1 ; c1ccc(-c2ccc(-c3ccc(N(c4ccc(-c5cc(-c6ccccc6)ccc5-c5ccccc5)cc4)c4ccc5c(ccc6ccccc65)c4)c4c3oc3ccccc34)cc2)cc1]

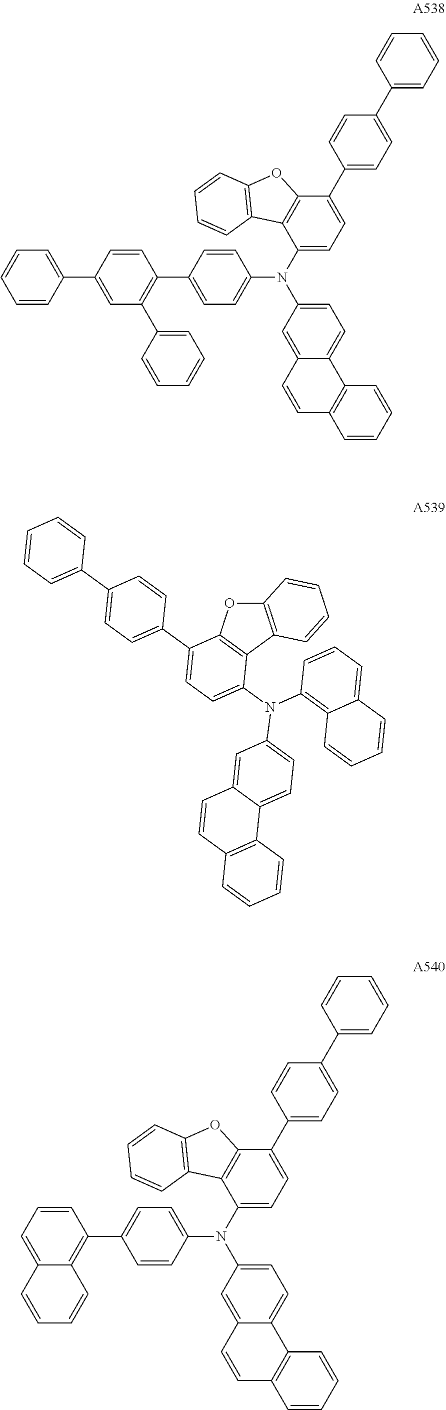 [SMILES: c1ccc(-c2ccc(-c3ccc(N(c4ccc(-c5ccc(-c6ccccc6)cc5-c5ccccc5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4cccc5ccccc45)c4c3oc3ccccc34)cc2)cc1 ; c1ccc(-c2ccc(-c3ccc(N(c4ccc(-c5cccc6ccccc56)cc4)c4ccc5c(ccc6ccccc65)c4)c4c3oc3ccccc34)cc2)cc1]

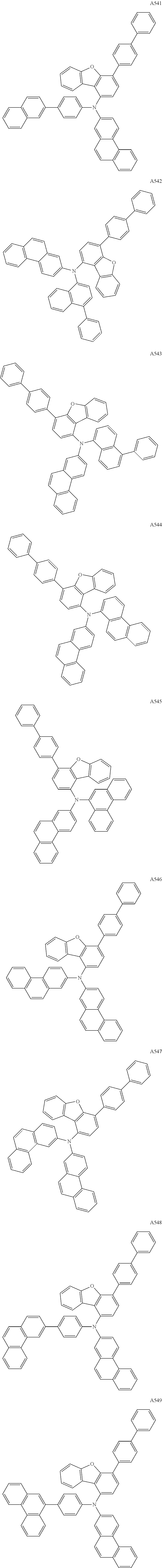

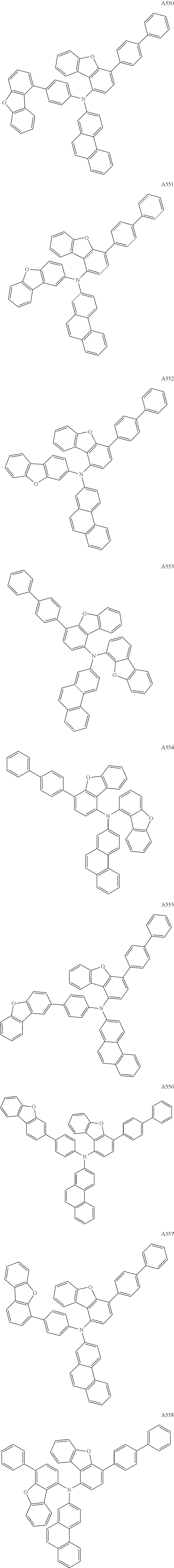

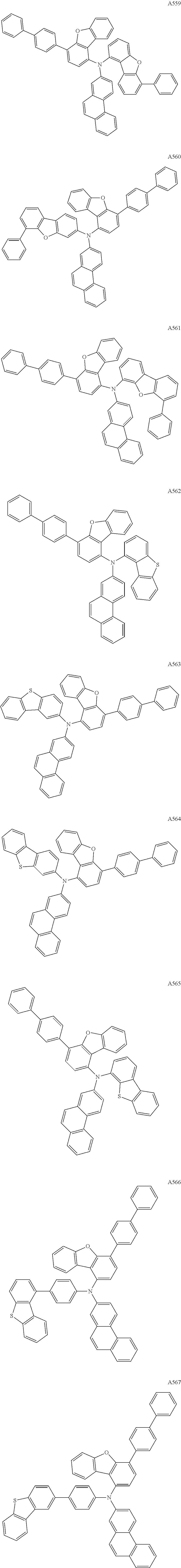

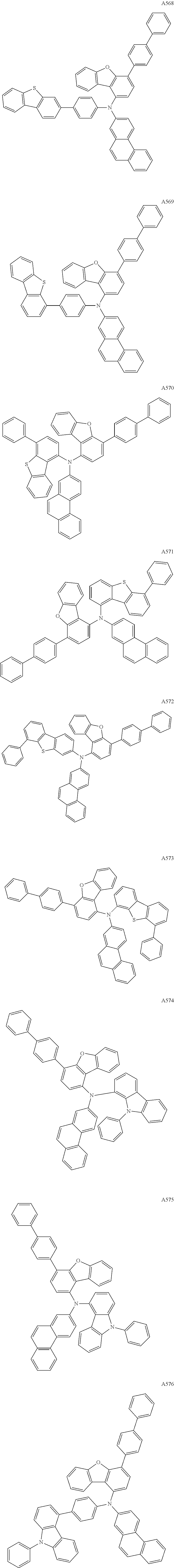

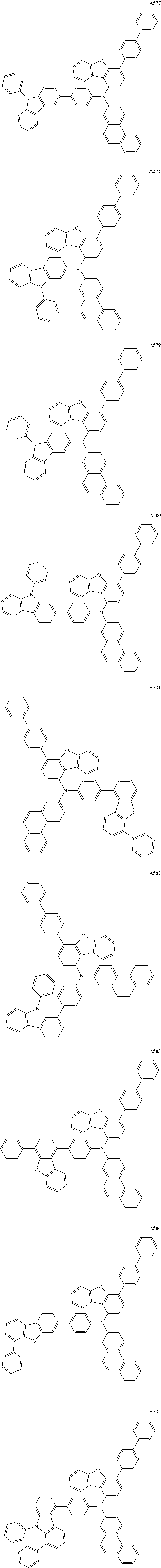

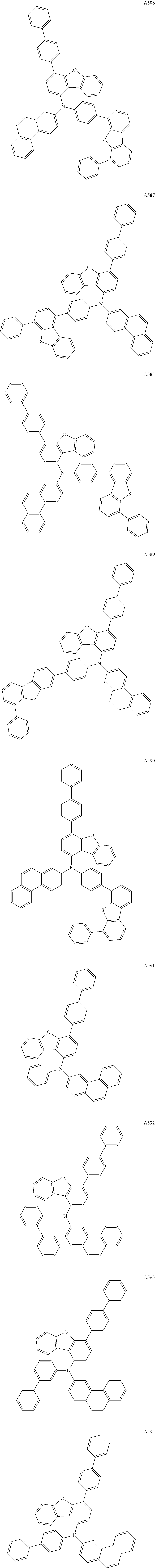

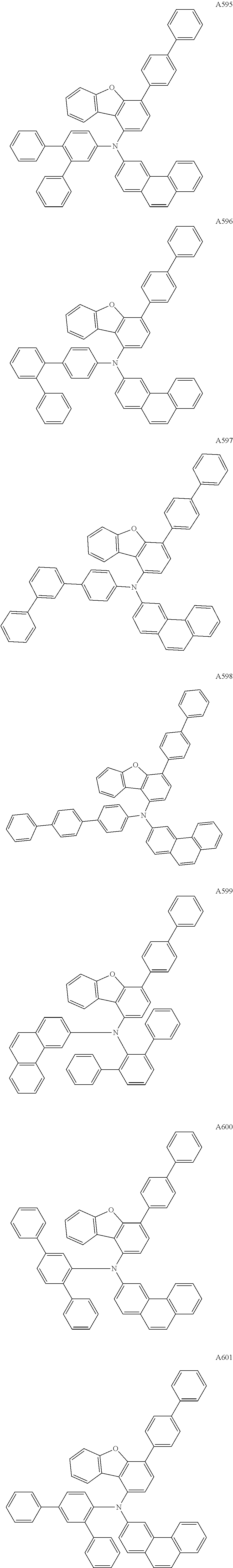

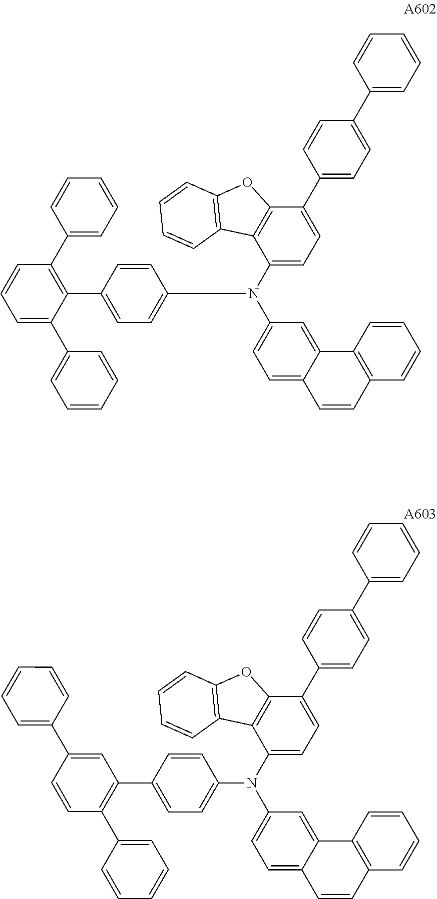 [SMILES: c1ccc(-c2ccc(-c3ccc(N(c4ccc(-c5c(-c6ccccc6)cccc5-c5ccccc5)cc4)c4ccc5ccc6ccccc6c5c4)c4c3oc3ccccc34)cc2)cc1 ; c1ccc(-c2ccc(-c3ccc(N(c4ccc(-c5cc(-c6ccccc6)ccc5-c5ccccc5)cc4)c4ccc5ccc6ccccc6c5c4)c4c3oc3ccccc34)cc2)cc1]

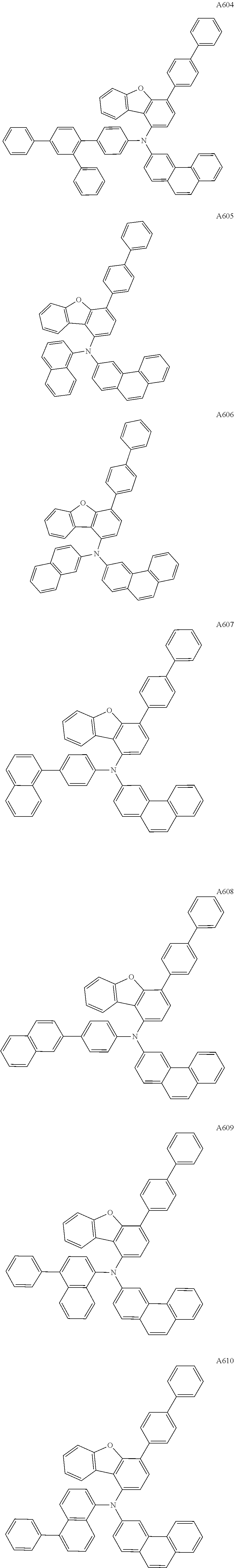

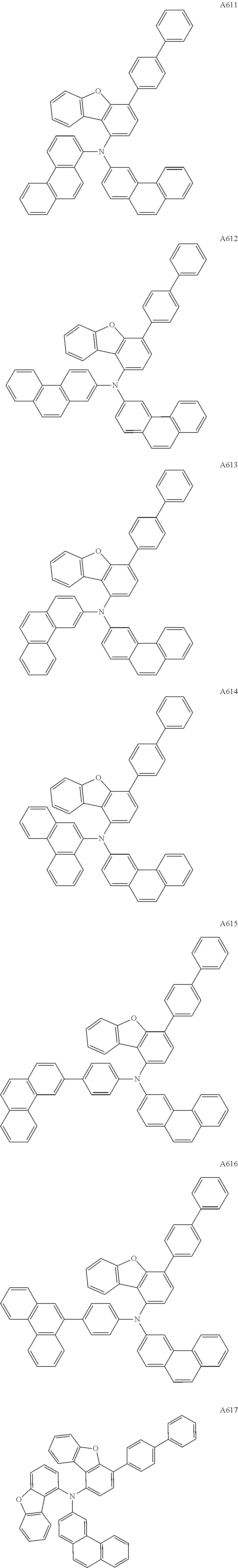

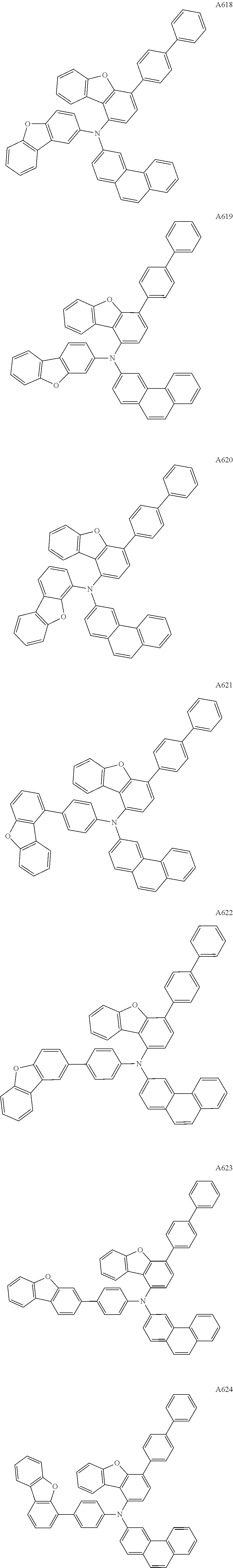

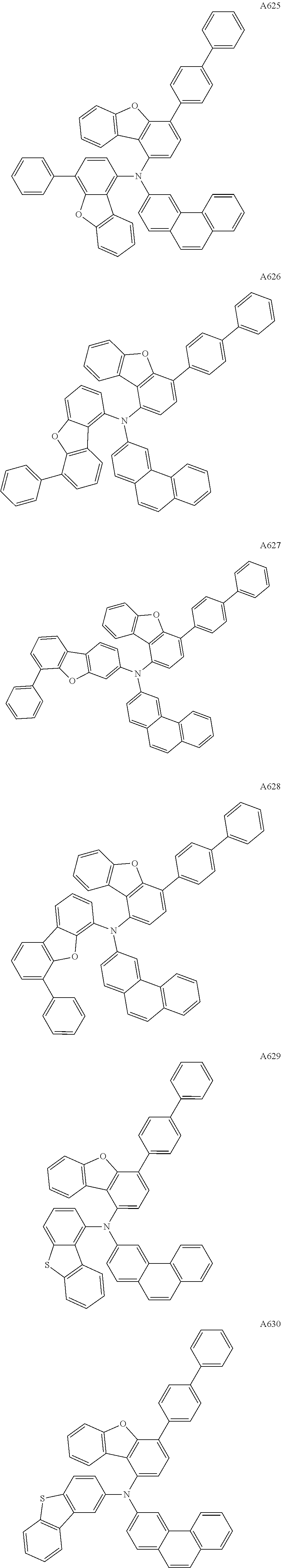

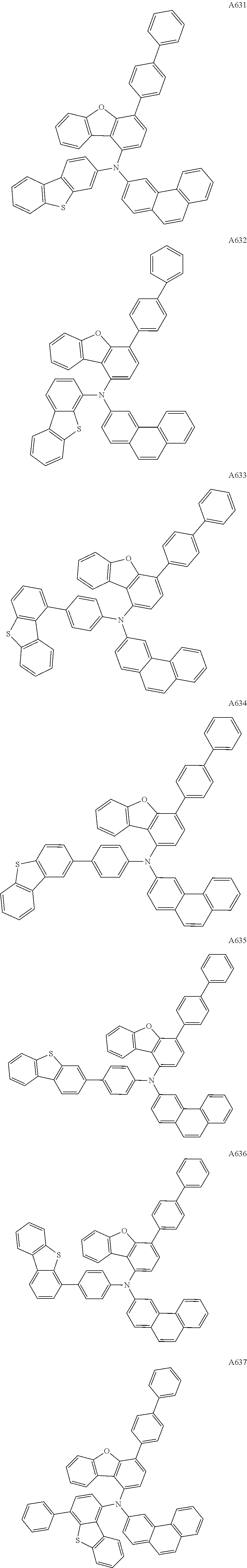

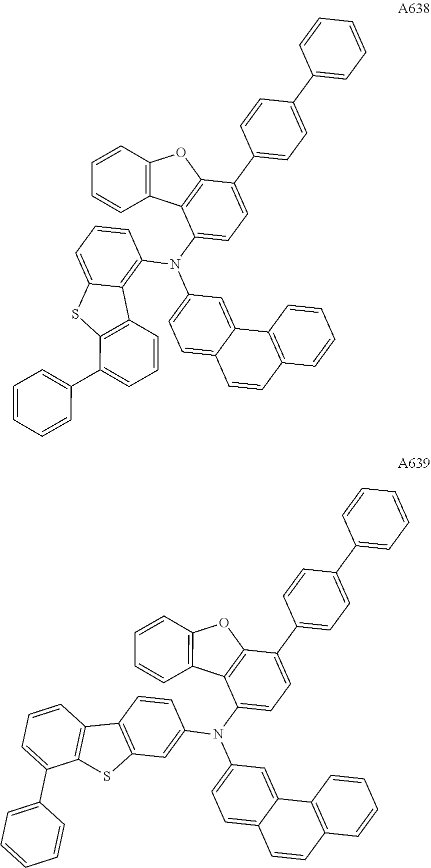 [SMILES: c1ccc(-c2ccc(-c3ccc(N(c4ccc5ccc6ccccc6c5c4)c4cccc5sc6c(-c7ccccc7)cccc6c45)c4c3oc3ccccc34)cc2)cc1 ; c1ccc(-c2ccc(-c3ccc(N(c4ccc5c(c4)sc4c(-c6ccccc6)cccc45)c4ccc5ccc6ccccc6c5c4)c4c3oc3ccccc34)cc2)cc1]

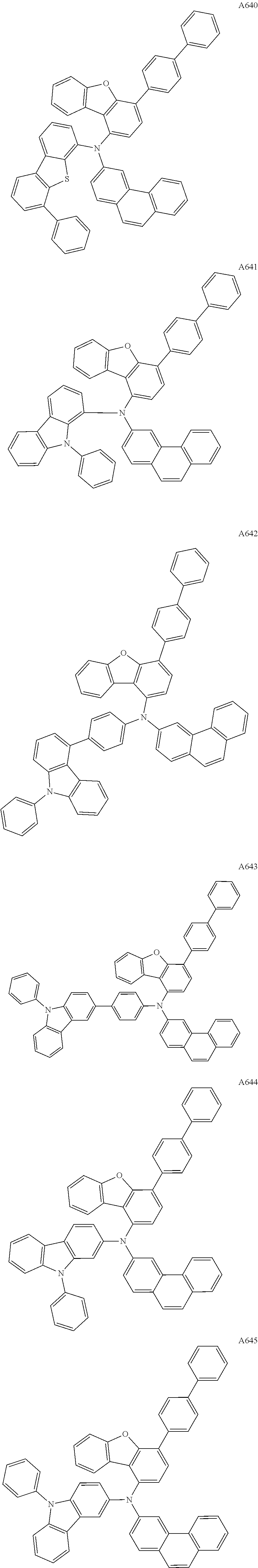

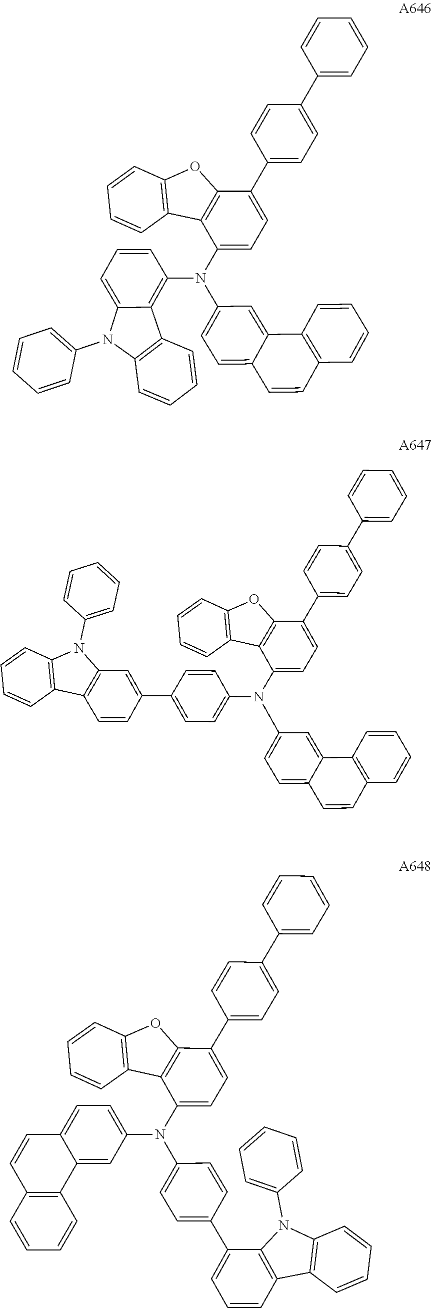 [SMILES: c1ccc(-c2ccc(-c3ccc(N(c4ccc5ccc6ccccc6c5c4)c4cccc5c4c4ccccc4n5-c4ccccc4)c4c3oc3ccccc34)cc2)cc1 ; c1ccc(-c2ccc(-c3ccc(N(c4ccc(-c5ccc6c7ccccc7n(-c7ccccc7)c6c5)cc4)c4ccc5ccc6ccccc6c5c4)c4c3oc3ccccc34)cc2)cc1 ; c1ccc(-c2ccc(-c3ccc(N(c4ccc(-c5cccc6c7ccccc7n(-c7ccccc7)c56)cc4)c4ccc5ccc6ccccc6c5c4)c4c3oc3ccccc34)cc2)cc1]

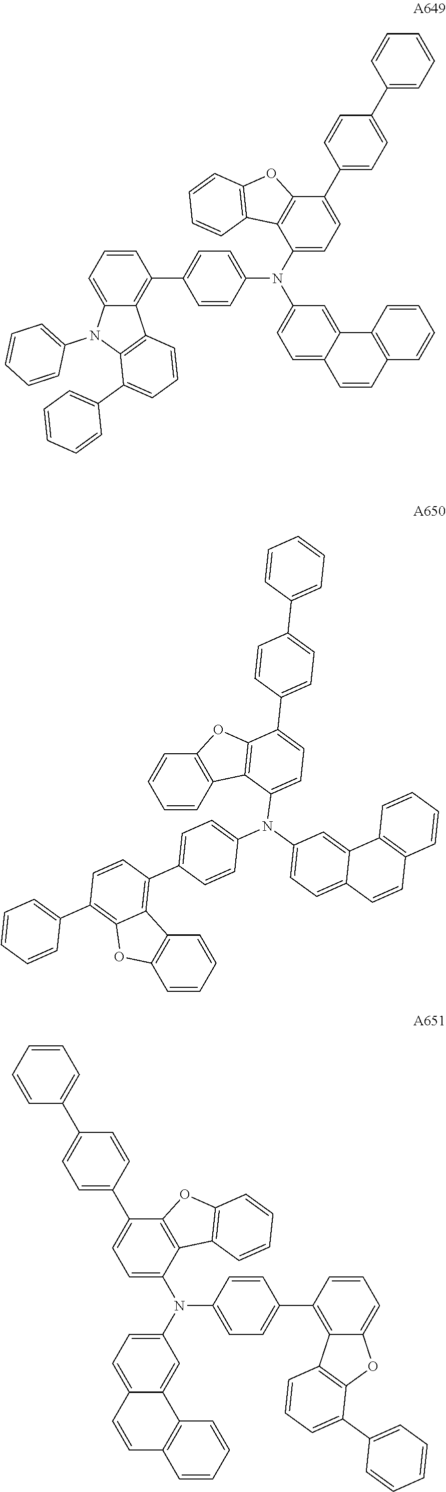 [SMILES: c1ccc(-c2ccc(-c3ccc(N(c4ccc(-c5cccc6c5c5cccc(-c7ccccc7)c5n6-c5ccccc5)cc4)c4ccc5ccc6ccccc6c5c4)c4c3oc3ccccc34)cc2)cc1 ; c1ccc(-c2ccc(-c3ccc(N(c4ccc(-c5ccc(-c6ccccc6)c6oc7ccccc7c56)cc4)c4ccc5ccc6ccccc6c5c4)c4c3oc3ccccc34)cc2)cc1 ; c1ccc(-c2ccc(-c3ccc(N(c4ccc(-c5cccc6oc7c(-c8ccccc8)cccc7c56)cc4)c4ccc5ccc6ccccc6c5c4)c4c3oc3ccccc34)cc2)cc1]

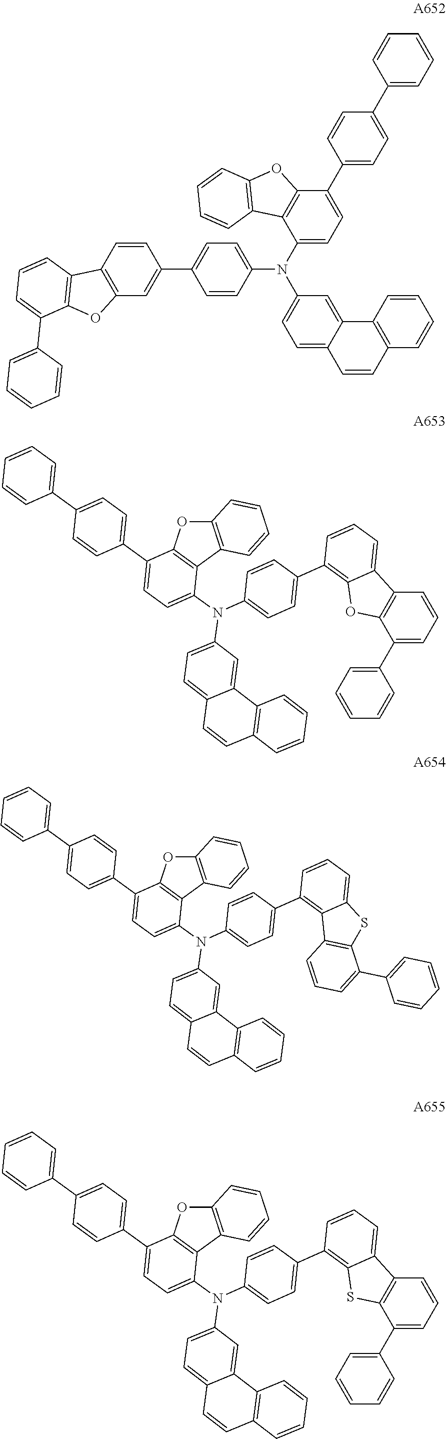

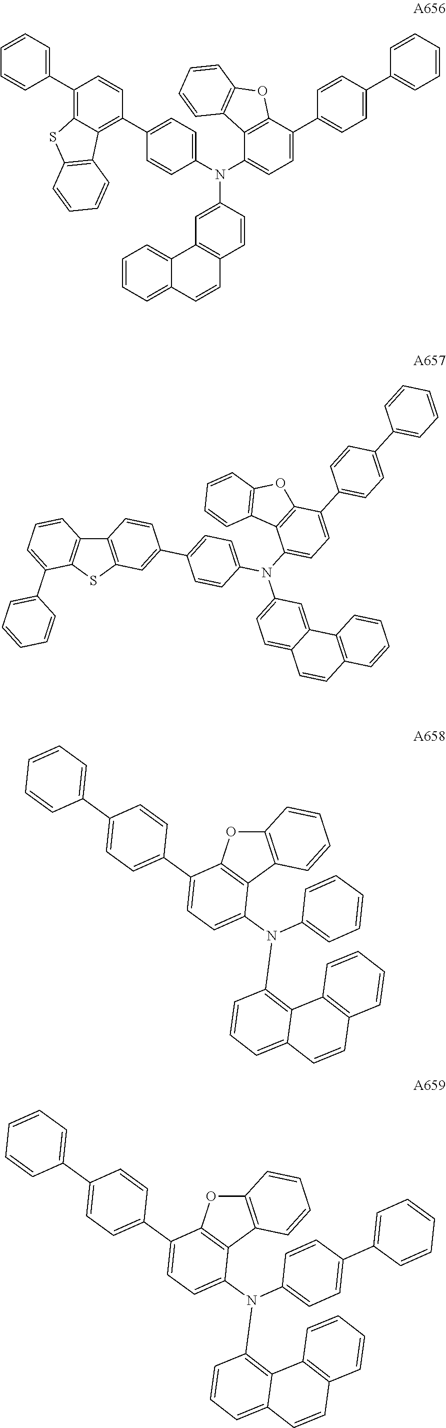 [SMILES: c1ccc(-c2ccc(-c3ccc(N(c4ccc(-c5ccc(-c6ccccc6)c6sc7ccccc7c56)cc4)c4ccc5ccc6ccccc6c5c4)c4c3oc3ccccc34)cc2)cc1 ; c1ccc(-c2ccc(-c3ccc(N(c4ccc(-c5ccc6c(c5)sc5c(-c7ccccc7)cccc56)cc4)c4ccc5ccc6ccccc6c5c4)c4c3oc3ccccc34)cc2)cc1 ; c1ccc(-c2ccc(-c3ccc(N(c4ccccc4)c4cccc5ccc6ccccc6c45)c4c3oc3ccccc34)cc2)cc1 ; c1ccc(-c2ccc(-c3ccc(N(c4ccc(-c5ccccc5)cc4)c4cccc5ccc6ccccc6c45)c4c3oc3ccccc34)cc2)cc1]

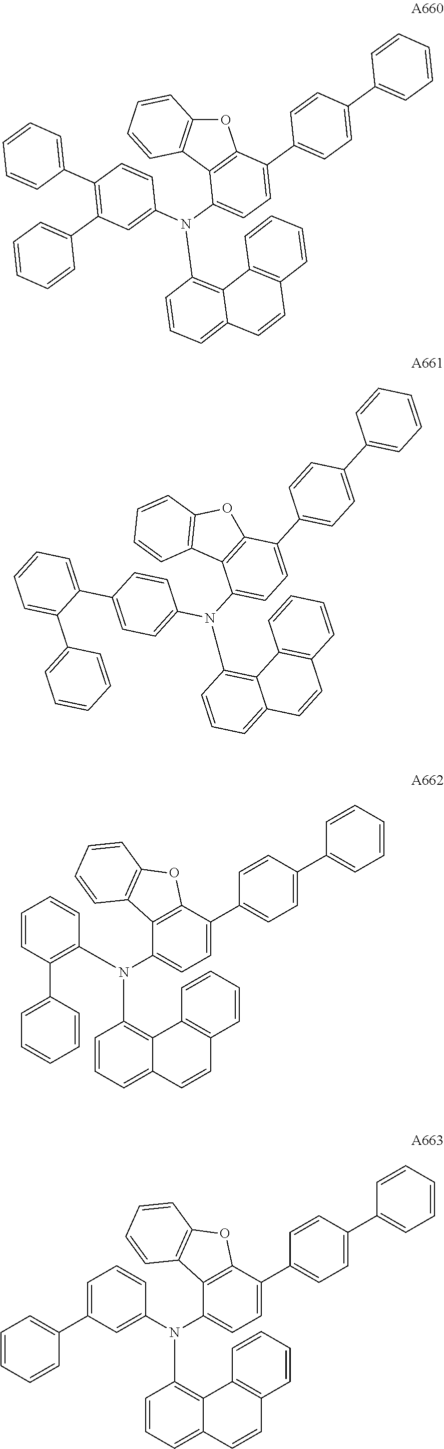 [SMILES: c1ccc(-c2ccc(-c3ccc(N(c4ccc(-c5ccccc5)c(-c5ccccc5)c4)c4cccc5ccc6ccccc6c45)c4c3oc3ccccc34)cc2)cc1 ; c1ccc(-c2ccc(-c3ccc(N(c4ccc(-c5ccccc5-c5ccccc5)cc4)c4cccc5ccc6ccccc6c45)c4c3oc3ccccc34)cc2)cc1 ; c1ccc(-c2ccc(-c3ccc(N(c4ccccc4-c4ccccc4)c4cccc5ccc6ccccc6c45)c4c3oc3ccccc34)cc2)cc1 ; c1ccc(-c2ccc(-c3ccc(N(c4cccc(-c5ccccc5)c4)c4cccc5ccc6ccccc6c45)c4c3oc3ccccc34)cc2)cc1]

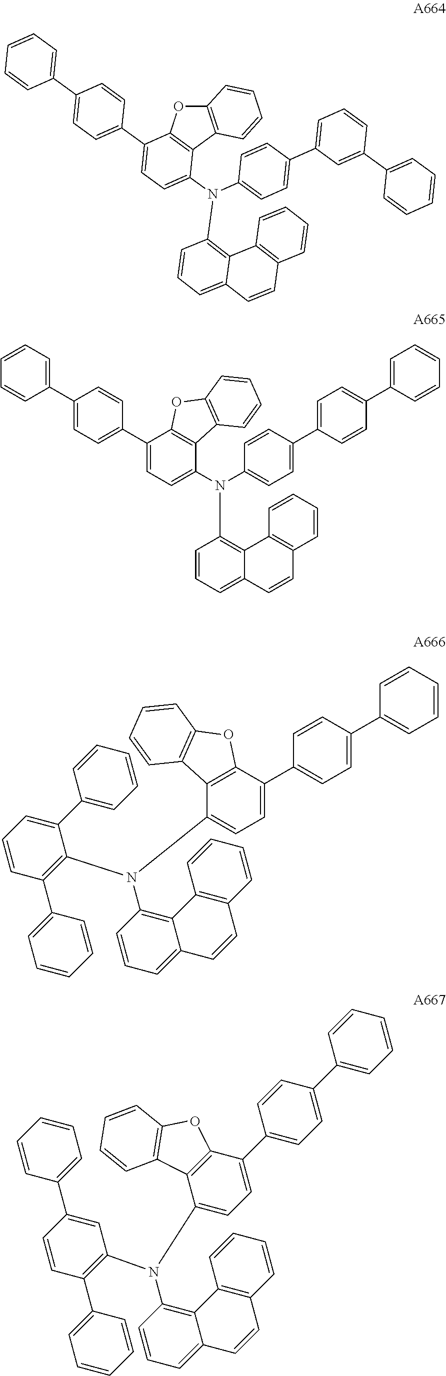 [SMILES: c1ccc(-c2ccc(-c3ccc(N(c4ccc(-c5cccc(-c6ccccc6)c5)cc4)c4cccc5ccc6ccccc6c45)c4c3oc3ccccc34)cc2)cc1 ; c1ccc(-c2ccc(-c3ccc(N(c4cccc5ccc6ccccc6c45)c4ccc(-c5ccc(-c6ccccc6)cc5)c5oc6ccccc6c45)cc3)cc2)cc1 ; c1ccc(-c2ccc(-c3ccc(N(c4c(-c5ccccc5)cccc4-c4ccccc4)c4cccc5ccc6ccccc6c45)c4c3oc3ccccc34)cc2)cc1 ; c1ccc(-c2ccc(-c3ccc(N(c4cc(-c5ccccc5)ccc4-c4ccccc4)c4cccc5ccc6ccccc6c45)c4c3oc3ccccc34)cc2)cc1]

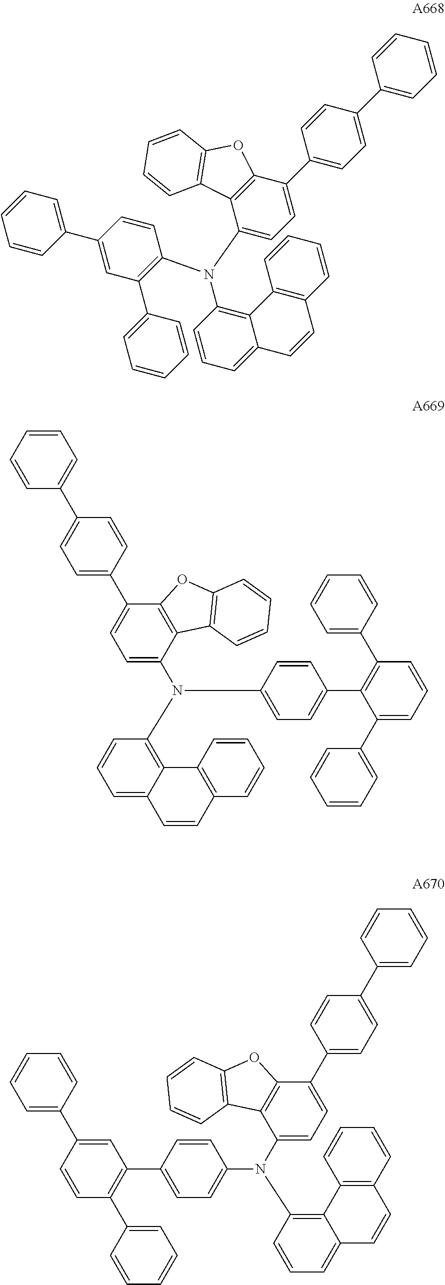 [SMILES: c1ccc(-c2ccc(-c3ccc(N(c4ccc(-c5ccccc5)cc4-c4ccccc4)c4cccc5ccc6ccccc6c45)c4c3oc3ccccc34)cc2)cc1 ; c1ccc(-c2ccc(-c3ccc(N(c4ccc(-c5c(-c6ccccc6)cccc5-c5ccccc5)cc4)c4cccc5ccc6ccccc6c45)c4c3oc3ccccc34)cc2)cc1 ; c1ccc(-c2ccc(-c3ccc(N(c4ccc(-c5cc(-c6ccccc6)ccc5-c5ccccc5)cc4)c4cccc5ccc6ccccc6c45)c4c3oc3ccccc34)cc2)cc1]

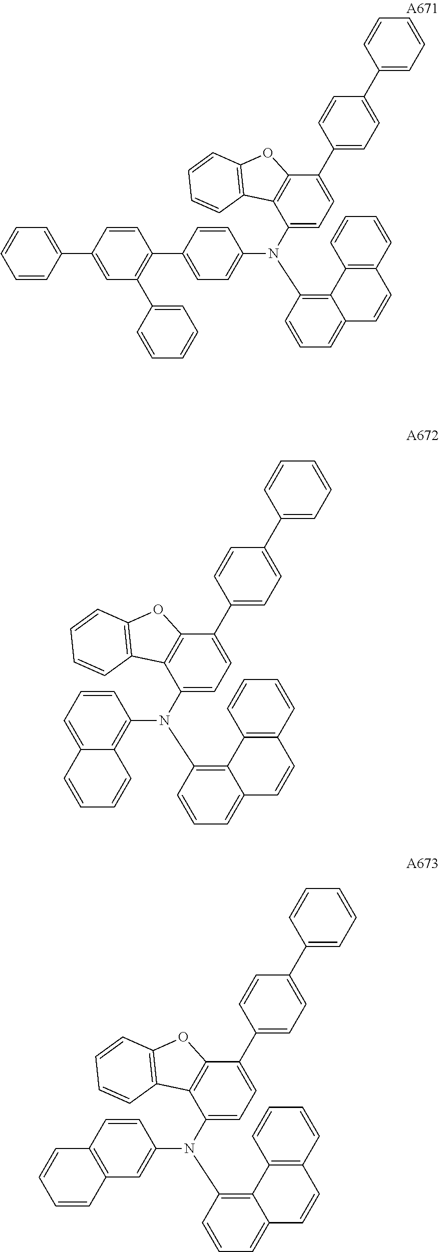 [SMILES: c1ccc(-c2ccc(-c3ccc(N(c4ccc(-c5ccc(-c6ccccc6)cc5-c5ccccc5)cc4)c4cccc5ccc6ccccc6c45)c4c3oc3ccccc34)cc2)cc1 ; c1ccc(-c2ccc(-c3ccc(N(c4cccc5ccccc45)c4cccc5ccc6ccccc6c45)c4c3oc3ccccc34)cc2)cc1 ; c1ccc(-c2ccc(-c3ccc(N(c4ccc5ccccc5c4)c4cccc5ccc6ccccc6c45)c4c3oc3ccccc34)cc2)cc1]

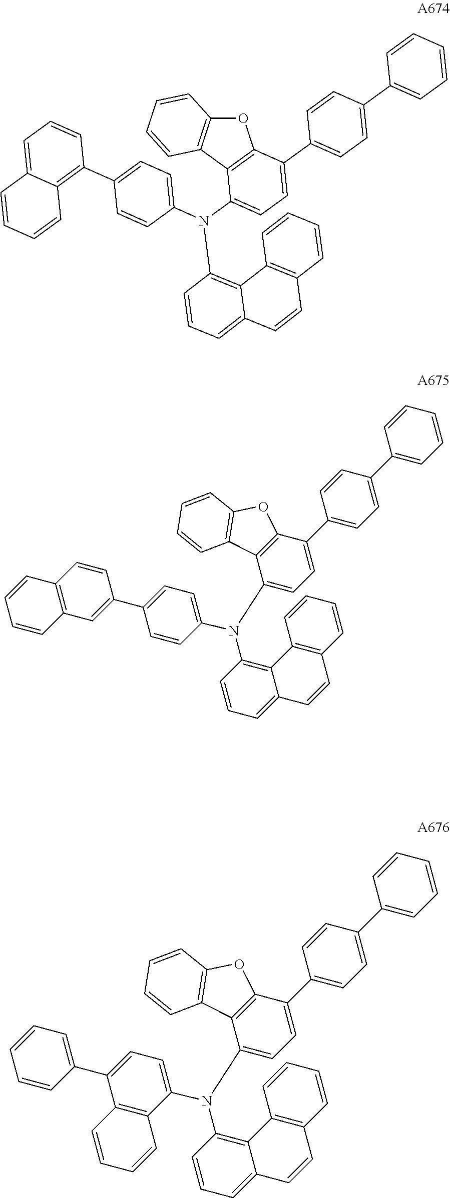 [SMILES: c1ccc(-c2ccc(-c3ccc(N(c4ccc(-c5cccc6ccccc56)cc4)c4cccc5ccc6ccccc6c45)c4c3oc3ccccc34)cc2)cc1 ; c1ccc(-c2ccc(-c3ccc(N(c4ccc(-c5ccc6ccccc6c5)cc4)c4cccc5ccc6ccccc6c45)c4c3oc3ccccc34)cc2)cc1 ; c1ccc(-c2ccc(-c3ccc(N(c4ccc(-c5ccccc5)c5ccccc45)c4cccc5ccc6ccccc6c45)c4c3oc3ccccc34)cc2)cc1]

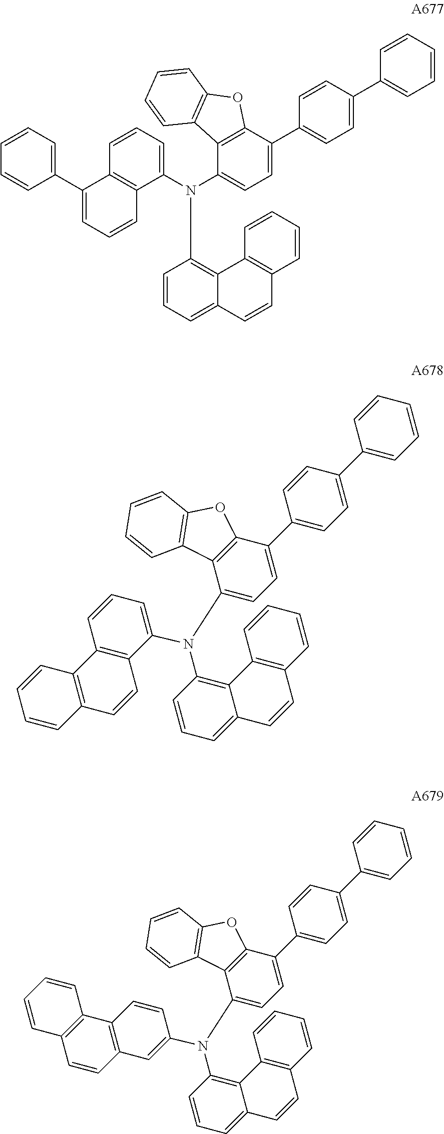 [SMILES: c1ccc(-c2ccc(-c3ccc(N(c4cccc5c(-c6ccccc6)cccc45)c4cccc5ccc6ccccc6c45)c4c3oc3ccccc34)cc2)cc1 ; c1ccc(-c2ccc(-c3ccc(N(c4cccc5c4ccc4ccccc45)c4cccc5ccc6ccccc6c45)c4c3oc3ccccc34)cc2)cc1 ; c1ccc(-c2ccc(-c3ccc(N(c4ccc5c(ccc6ccccc65)c4)c4cccc5ccc6ccccc6c45)c4c3oc3ccccc34)cc2)cc1]

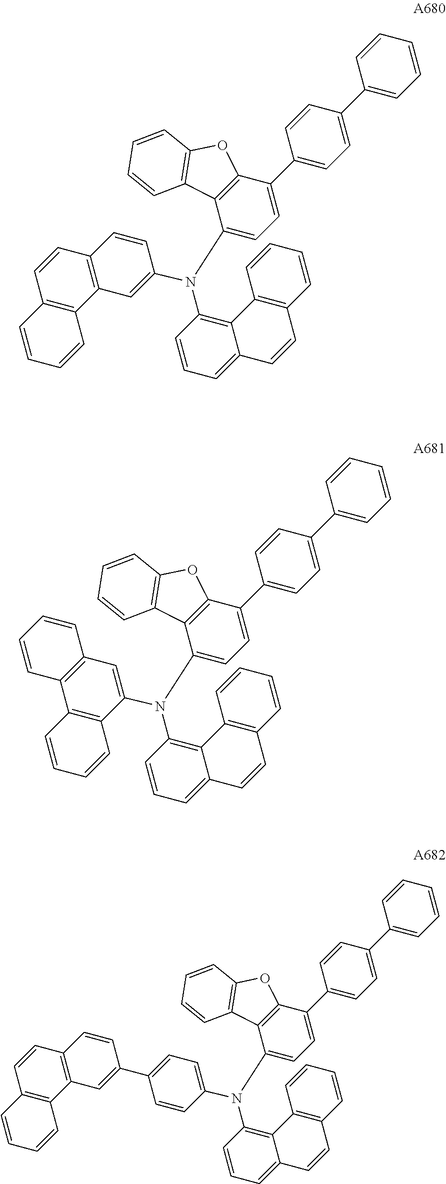 [SMILES: c1ccc(-c2ccc(-c3ccc(N(c4ccc5ccc6ccccc6c5c4)c4cccc5ccc6ccccc6c45)c4c3oc3ccccc34)cc2)cc1 ; c1ccc(-c2ccc(-c3ccc(N(c4cc5ccccc5c5ccccc45)c4cccc5ccc6ccccc6c45)c4c3oc3ccccc34)cc2)cc1 ; c1ccc(-c2ccc(-c3ccc(N(c4ccc(-c5ccc6ccc7ccccc7c6c5)cc4)c4cccc5ccc6ccccc6c45)c4c3oc3ccccc34)cc2)cc1]

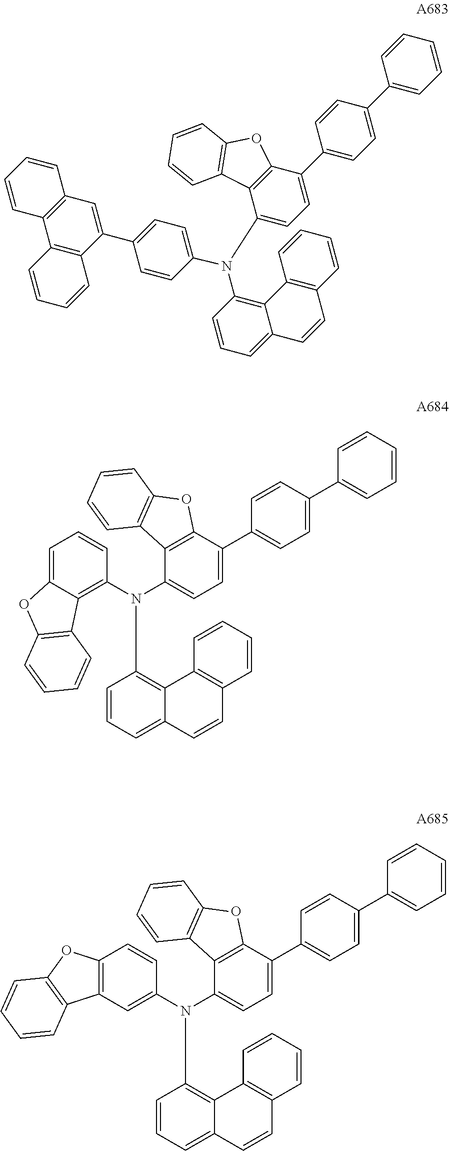 [SMILES: c1ccc(-c2ccc(-c3ccc(N(c4ccc(-c5cc6ccccc6c6ccccc56)cc4)c4cccc5ccc6ccccc6c45)c4c3oc3ccccc34)cc2)cc1 ; c1ccc(-c2ccc(-c3ccc(N(c4cccc5ccc6ccccc6c45)c4cccc5oc6ccccc6c45)c4c3oc3ccccc34)cc2)cc1 ; c1ccc(-c2ccc(-c3ccc(N(c4ccc5oc6ccccc6c5c4)c4cccc5ccc6ccccc6c45)c4c3oc3ccccc34)cc2)cc1]

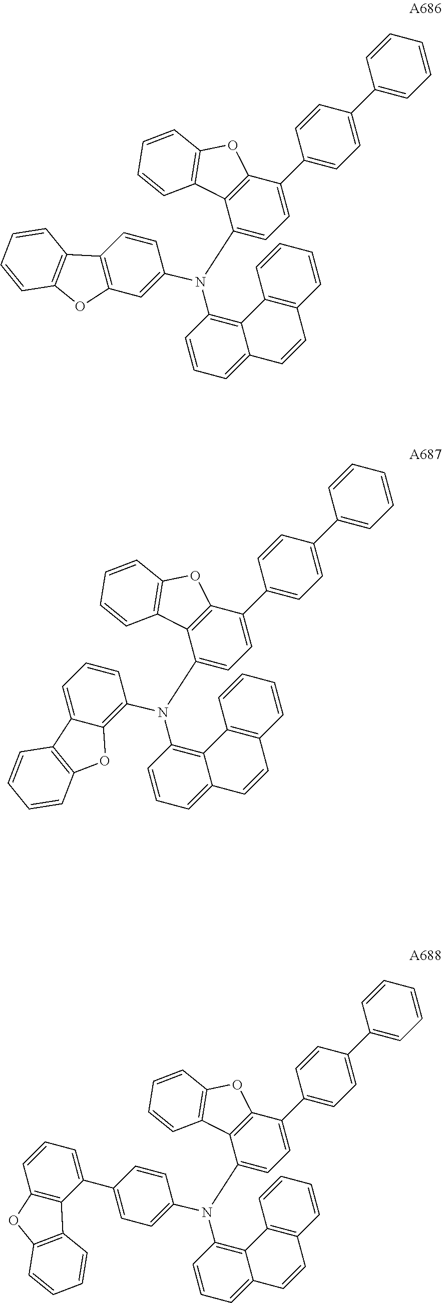 [SMILES: c1ccc(-c2ccc(-c3ccc(N(c4ccc5c(c4)oc4ccccc45)c4cccc5ccc6ccccc6c45)c4c3oc3ccccc34)cc2)cc1 ; c1ccc(-c2ccc(-c3ccc(N(c4cccc5c4oc4ccccc45)c4cccc5ccc6ccccc6c45)c4c3oc3ccccc34)cc2)cc1 ; c1ccc(-c2ccc(-c3ccc(N(c4ccc(-c5cccc6oc7ccccc7c56)cc4)c4cccc5ccc6ccccc6c45)c4c3oc3ccccc34)cc2)cc1]

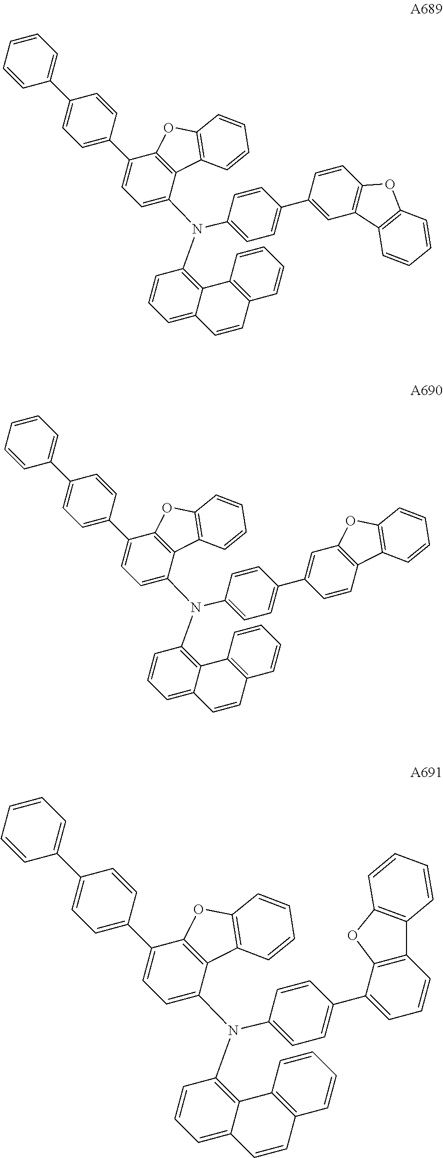 [SMILES: c1ccc(-c2ccc(-c3ccc(N(c4ccc(-c5ccc6oc7ccccc7c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)oc5ccccc56)cc4)c4cccc5ccc6ccccc6c45)c4c3oc3ccccc34)cc2)cc1 ; c1ccc(-c2ccc(-c3ccc(N(c4ccc(-c5cccc6c5oc5ccccc56)cc4)c4cccc5ccc6ccccc6c45)c4c3oc3ccccc34)cc2)cc1]

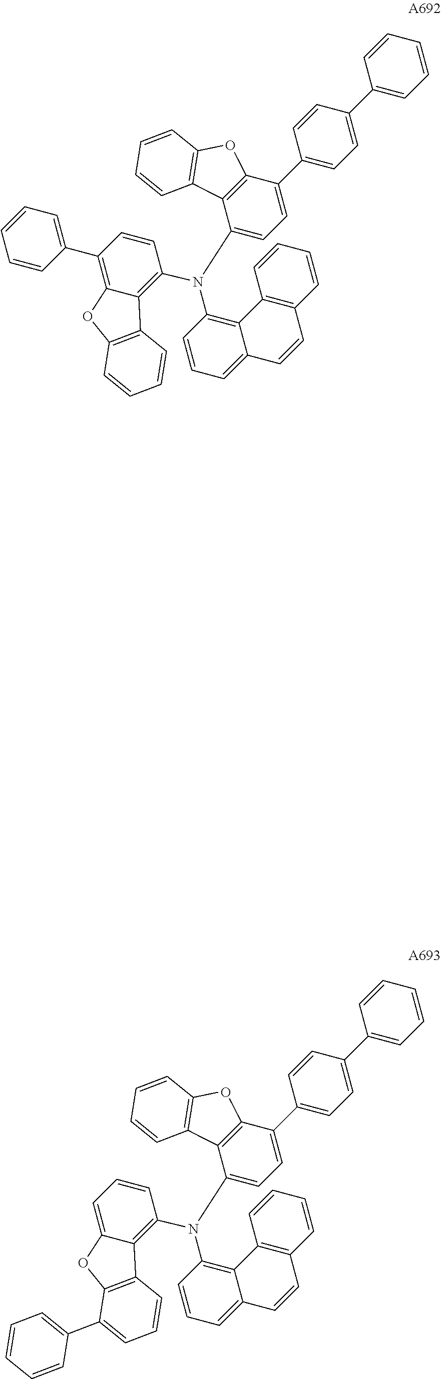 [SMILES: c1ccc(-c2ccc(-c3ccc(N(c4cccc5ccc6ccccc6c45)c4ccc(-c5ccccc5)c5oc6ccccc6c45)c4c3oc3ccccc34)cc2)cc1 ; c1ccc(-c2ccc(-c3ccc(N(c4cccc5ccc6ccccc6c45)c4cccc5oc6c(-c7ccccc7)cccc6c45)c4c3oc3ccccc34)cc2)cc1]

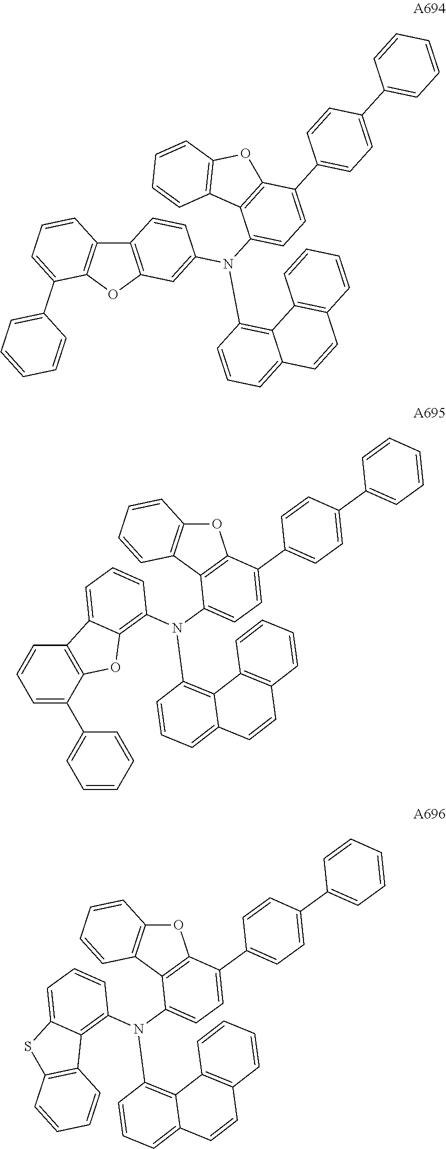 [SMILES: c1ccc(-c2ccc(-c3ccc(N(c4ccc5c(c4)oc4c(-c6ccccc6)cccc45)c4cccc5ccc6ccccc6c45)c4c3oc3ccccc34)cc2)cc1 ; c1ccc(-c2ccc(-c3ccc(N(c4cccc5c4oc4c(-c6ccccc6)cccc45)c4cccc5ccc6ccccc6c45)c4c3oc3ccccc34)cc2)cc1 ; c1ccc(-c2ccc(-c3ccc(N(c4cccc5ccc6ccccc6c45)c4cccc5sc6ccccc6c45)c4c3oc3ccccc34)cc2)cc1]

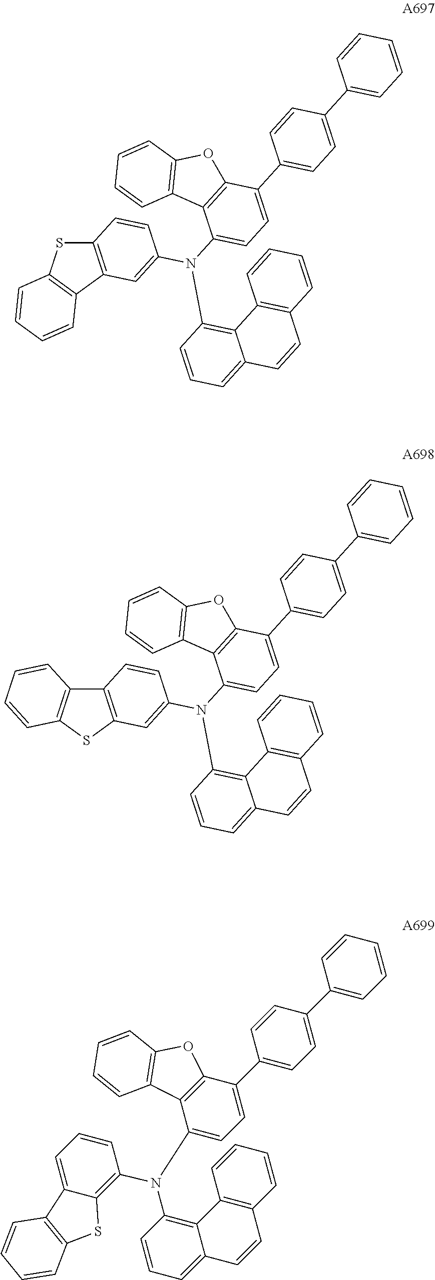 [SMILES: c1ccc(-c2ccc(-c3ccc(N(c4ccc5sc6ccccc6c5c4)c4cccc5ccc6ccccc6c45)c4c3oc3ccccc34)cc2)cc1 ; c1ccc(-c2ccc(-c3ccc(N(c4ccc5c(c4)sc4ccccc45)c4cccc5ccc6ccccc6c45)c4c3oc3ccccc34)cc2)cc1 ; c1ccc(-c2ccc(-c3ccc(N(c4cccc5c4sc4ccccc45)c4cccc5ccc6ccccc6c45)c4c3oc3ccccc34)cc2)cc1]

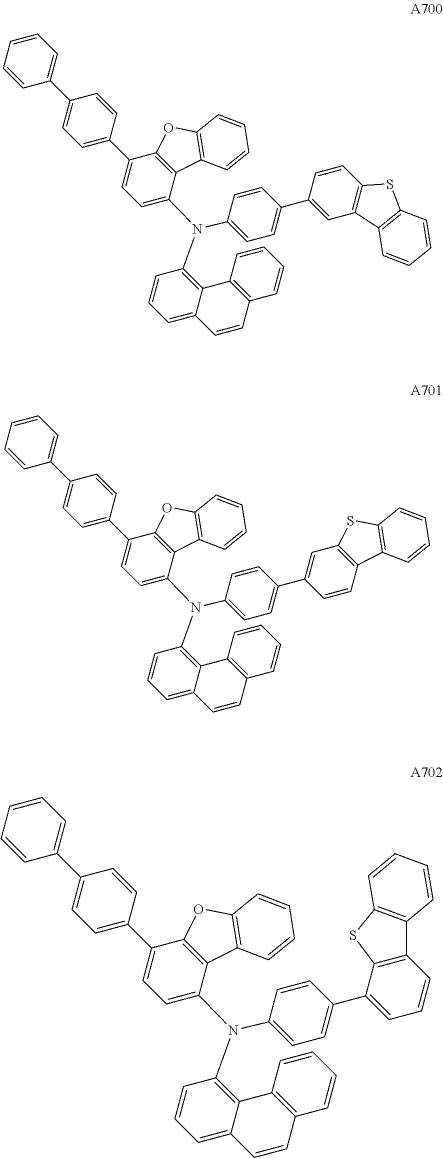 [SMILES: c1ccc(-c2ccc(-c3ccc(N(c4ccc(-c5ccc6sc7ccccc7c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)sc5ccccc56)cc4)c4cccc5ccc6ccccc6c45)c4c3oc3ccccc34)cc2)cc1 ; c1ccc(-c2ccc(-c3ccc(N(c4ccc(-c5cccc6c5sc5ccccc56)cc4)c4cccc5ccc6ccccc6c45)c4c3oc3ccccc34)cc2)cc1]

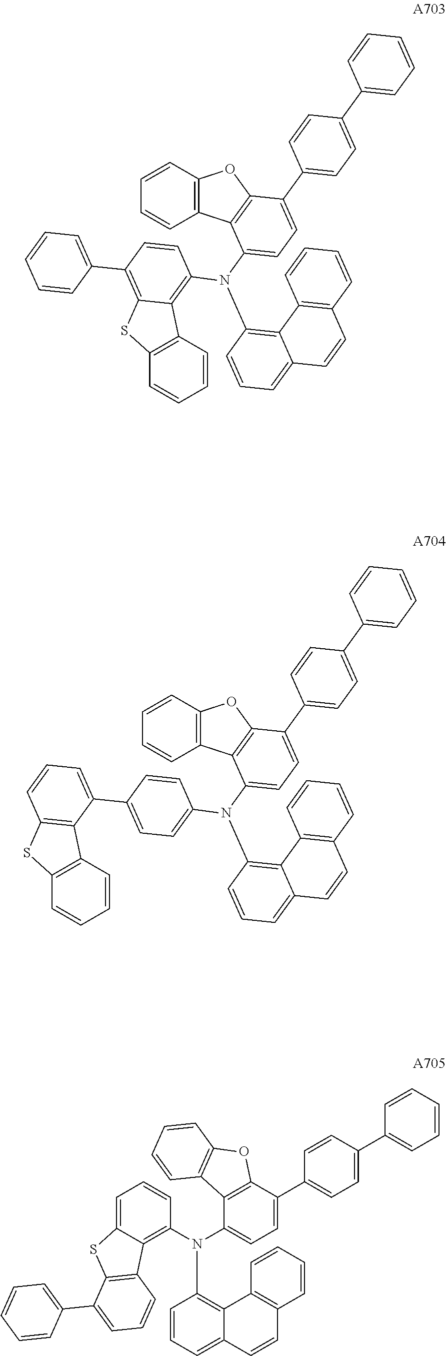 [SMILES: c1ccc(-c2ccc(-c3ccc(N(c4cccc5ccc6ccccc6c45)c4ccc(-c5ccccc5)c5sc6ccccc6c45)c4c3oc3ccccc34)cc2)cc1 ; c1ccc(-c2ccc(-c3ccc(N(c4ccc(-c5cccc6sc7ccccc7c56)cc4)c4cccc5ccc6ccccc6c45)c4c3oc3ccccc34)cc2)cc1 ; c1ccc(-c2ccc(-c3ccc(N(c4cccc5ccc6ccccc6c45)c4cccc5sc6c(-c7ccccc7)cccc6c45)c4c3oc3ccccc34)cc2)cc1]

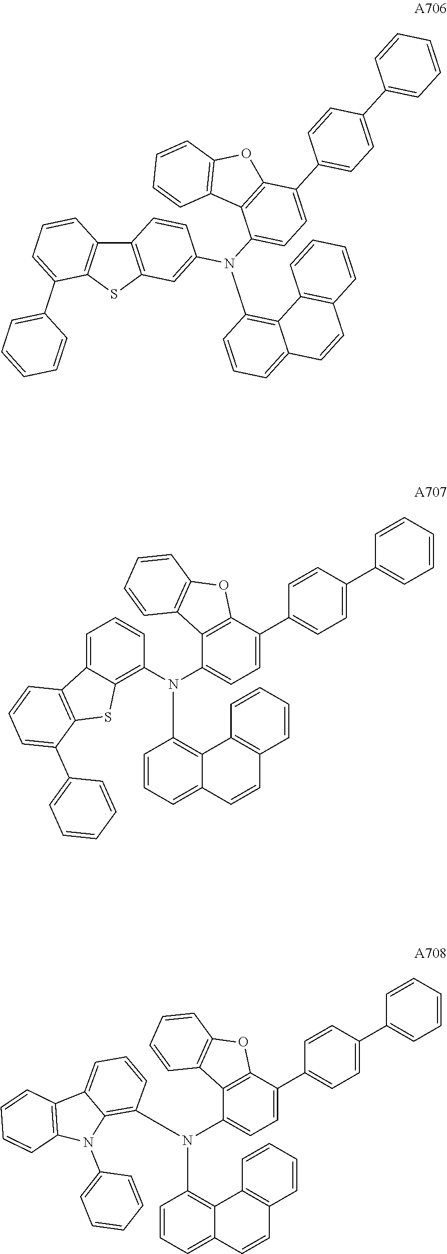 [SMILES: c1ccc(-c2ccc(-c3ccc(N(c4ccc5c(c4)sc4c(-c6ccccc6)cccc45)c4cccc5ccc6ccccc6c45)c4c3oc3ccccc34)cc2)cc1 ; c1ccc(-c2ccc(-c3ccc(N(c4cccc5c4sc4c(-c6ccccc6)cccc45)c4cccc5ccc6ccccc6c45)c4c3oc3ccccc34)cc2)cc1 ; c1ccc(-c2ccc(-c3ccc(N(c4cccc5ccc6ccccc6c45)c4cccc5c6ccccc6n(-c6ccccc6)c45)c4c3oc3ccccc34)cc2)cc1]

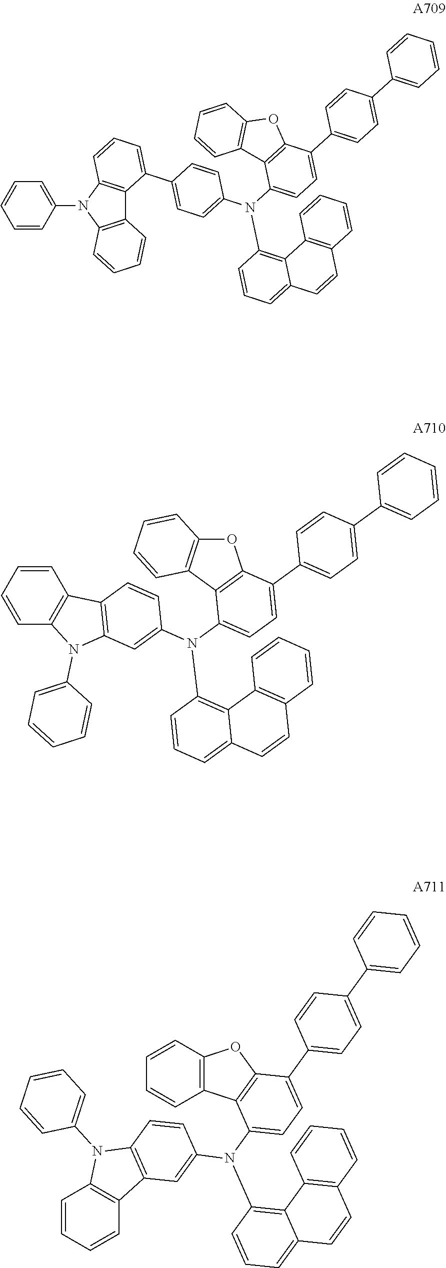 [SMILES: c1ccc(-c2ccc(-c3ccc(N(c4ccc(-c5cccc6c5c5ccccc5n6-c5ccccc5)cc4)c4cccc5ccc6ccccc6c45)c4c3oc3ccccc34)cc2)cc1 ; c1ccc(-c2ccc(-c3ccc(N(c4ccc5c6ccccc6n(-c6ccccc6)c5c4)c4cccc5ccc6ccccc6c45)c4c3oc3ccccc34)cc2)cc1 ; c1ccc(-c2ccc(-c3ccc(N(c4ccc5c(c4)c4ccccc4n5-c4ccccc4)c4cccc5ccc6ccccc6c45)c4c3oc3ccccc34)cc2)cc1]

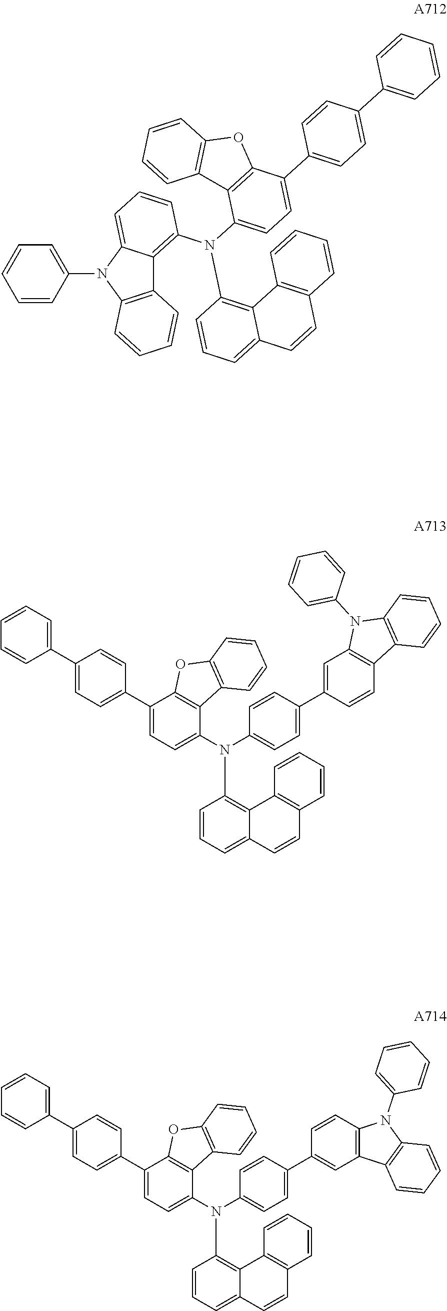 [SMILES: c1ccc(-c2ccc(-c3ccc(N(c4cccc5ccc6ccccc6c45)c4cccc5c4c4ccccc4n5-c4ccccc4)c4c3oc3ccccc34)cc2)cc1 ; c1ccc(-c2ccc(-c3ccc(N(c4ccc(-c5ccc6c7ccccc7n(-c7ccccc7)c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)c5ccccc5n6-c5ccccc5)cc4)c4cccc5ccc6ccccc6c45)c4c3oc3ccccc34)cc2)cc1]

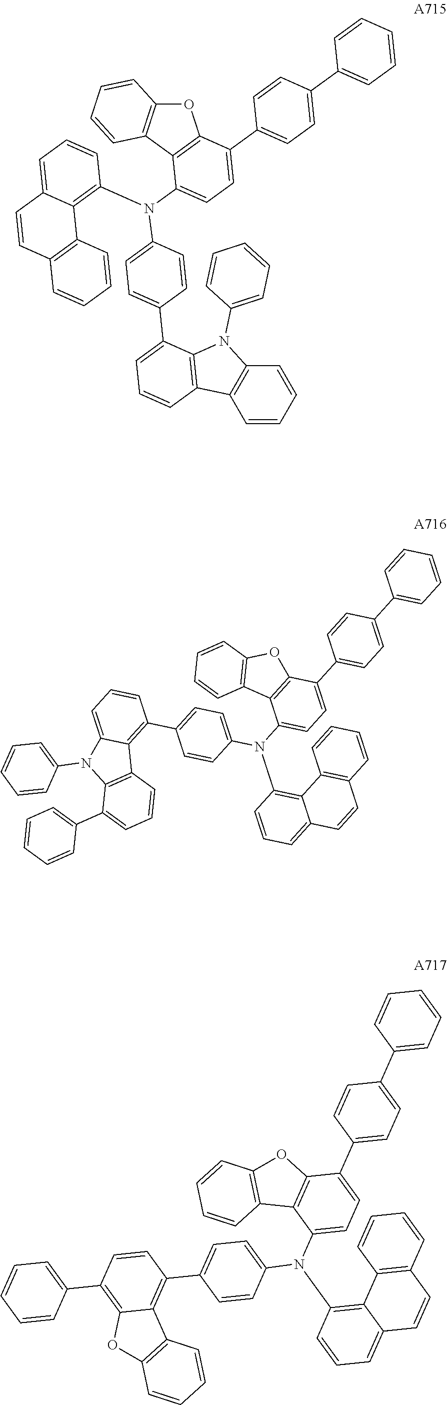 [SMILES: c1ccc(-c2ccc(-c3ccc(N(c4ccc(-c5cccc6c7ccccc7n(-c7ccccc7)c56)cc4)c4cccc5ccc6ccccc6c45)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4cccc5ccc6ccccc6c45)c4c3oc3ccccc34)cc2)cc1 ; c1ccc(-c2ccc(-c3ccc(N(c4ccc(-c5ccc(-c6ccccc6)c6oc7ccccc7c56)cc4)c4cccc5ccc6ccccc6c45)c4c3oc3ccccc34)cc2)cc1]

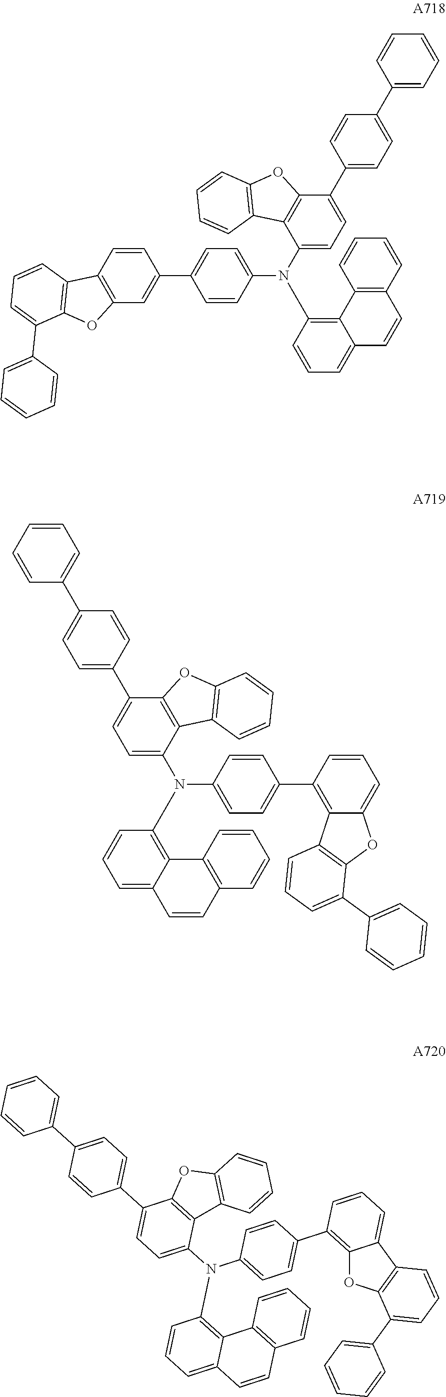 [SMILES: c1ccc(-c2ccc(-c3ccc(N(c4ccc(-c5ccc6c(c5)oc5c(-c7ccccc7)cccc56)cc4)c4cccc5ccc6ccccc6c45)c4c3oc3ccccc34)cc2)cc1 ; c1ccc(-c2ccc(-c3ccc(N(c4ccc(-c5cccc6oc7c(-c8ccccc8)cccc7c56)cc4)c4cccc5ccc6ccccc6c45)c4c3oc3ccccc34)cc2)cc1 ; c1ccc(-c2ccc(-c3ccc(N(c4ccc(-c5cccc6c5oc5c(-c7ccccc7)cccc56)cc4)c4cccc5ccc6ccccc6c45)c4c3oc3ccccc34)cc2)cc1]

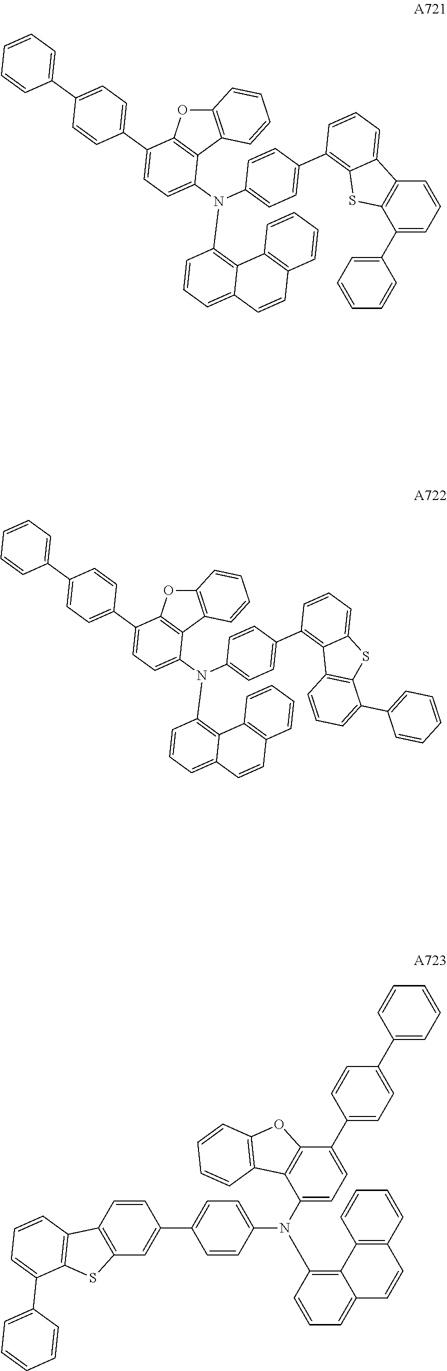 [SMILES: c1ccc(-c2ccc(-c3ccc(N(c4ccc(-c5cccc6c5sc5c(-c7ccccc7)cccc56)cc4)c4cccc5ccc6ccccc6c45)c4c3oc3ccccc34)cc2)cc1 ; c1ccc(-c2ccc(-c3ccc(N(c4ccc(-c5cccc6sc7c(-c8ccccc8)cccc7c56)cc4)c4cccc5ccc6ccccc6c45)c4c3oc3ccccc34)cc2)cc1 ; c1ccc(-c2ccc(-c3ccc(N(c4ccc(-c5ccc6c(c5)sc5c(-c7ccccc7)cccc56)cc4)c4cccc5ccc6ccccc6c45)c4c3oc3ccccc34)cc2)cc1]

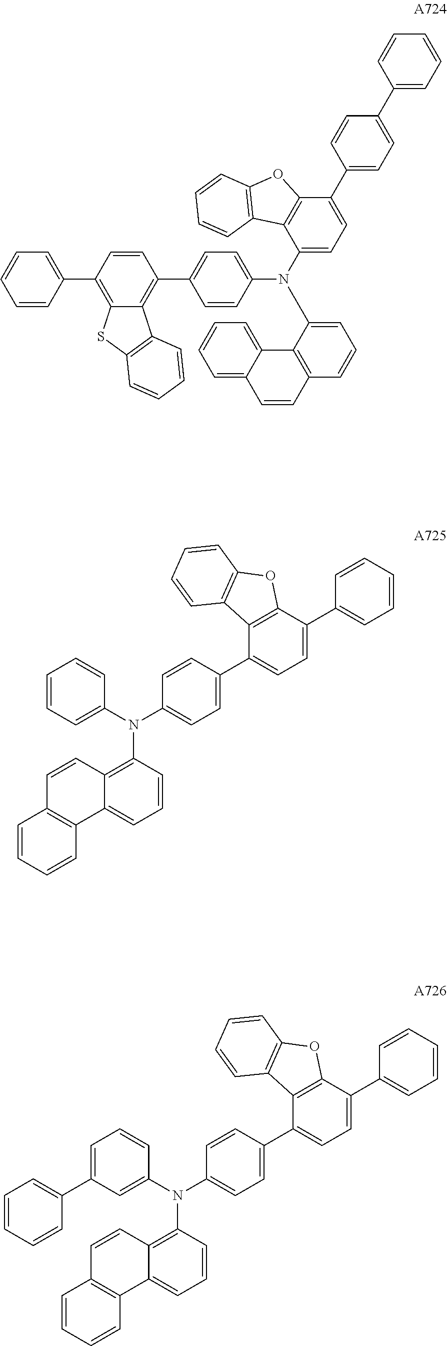 [SMILES: c1ccc(-c2ccc(-c3ccc(N(c4ccc(-c5ccc(-c6ccccc6)c6sc7ccccc7c56)cc4)c4cccc5ccc6ccccc6c45)c4c3oc3ccccc34)cc2)cc1 ; c1ccc(-c2ccc(-c3ccc(N(c4ccccc4)c4cccc5c4ccc4ccccc45)cc3)c3c2oc2ccccc23)cc1 ; c1ccc(-c2cccc(N(c3ccc(-c4ccc(-c5ccccc5)c5oc6ccccc6c45)cc3)c3cccc4c3ccc3ccccc34)c2)cc1]

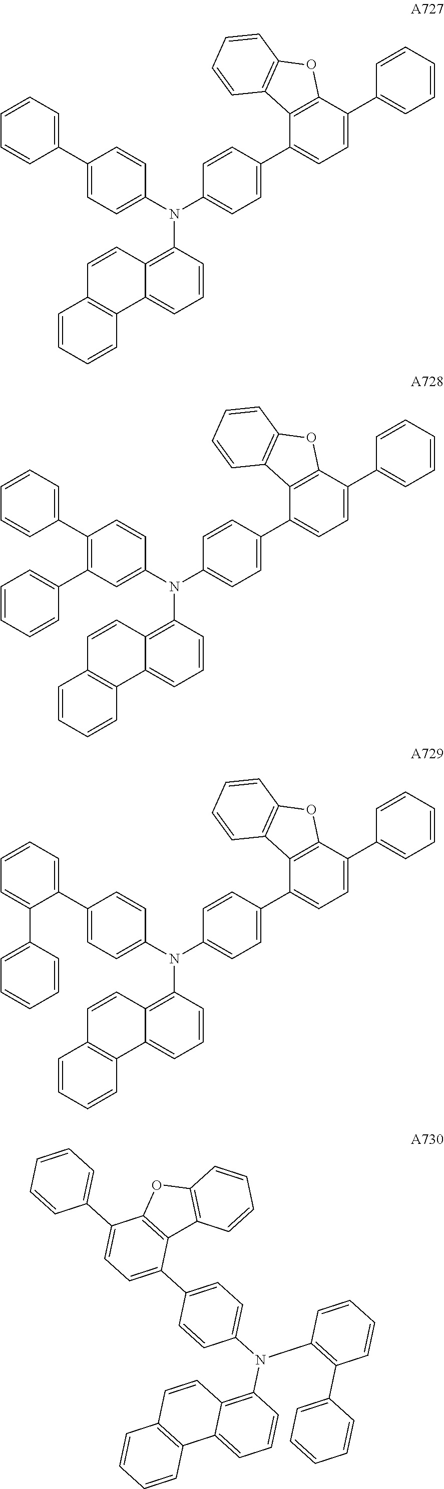

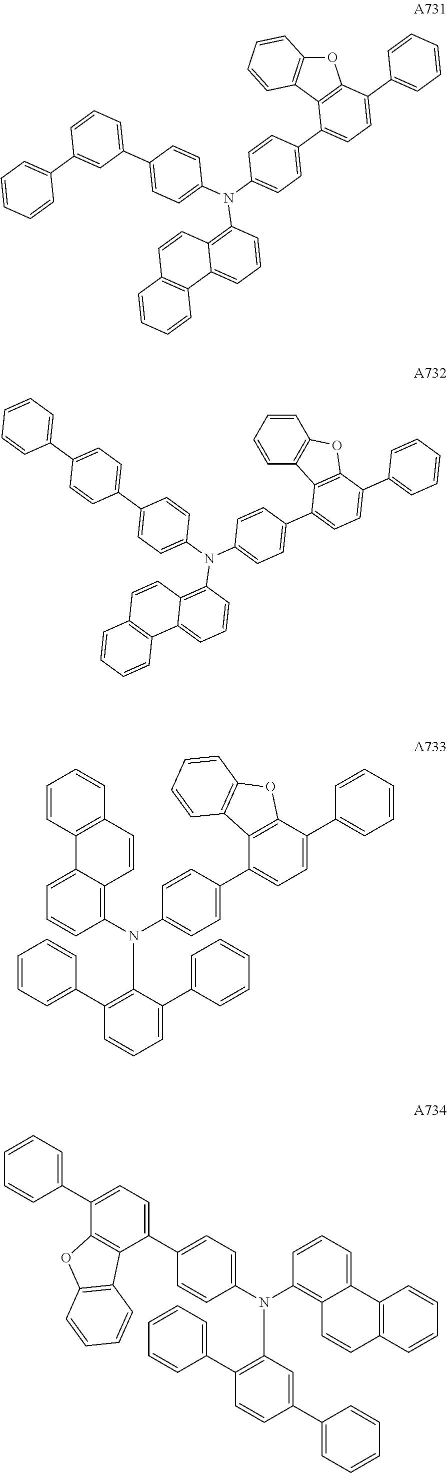 [SMILES: c1ccc(-c2cccc(-c3ccc(N(c4ccc(-c5ccc(-c6ccccc6)c6oc7ccccc7c56)cc4)c4cccc5c4ccc4ccccc45)cc3)c2)cc1 ; c1ccc(-c2ccc(-c3ccc(N(c4ccc(-c5ccc(-c6ccccc6)c6oc7ccccc7c56)cc4)c4cccc5c4ccc4ccccc45)cc3)cc2)cc1 ; c1ccc(-c2cccc(-c3ccccc3)c2N(c2ccc(-c3ccc(-c4ccccc4)c4oc5ccccc5c34)cc2)c2cccc3c2ccc2ccccc23)cc1 ; c1ccc(-c2ccc(-c3ccccc3)c(N(c3ccc(-c4ccc(-c5ccccc5)c5oc6ccccc6c45)cc3)c3cccc4c3ccc3ccccc34)c2)cc1]

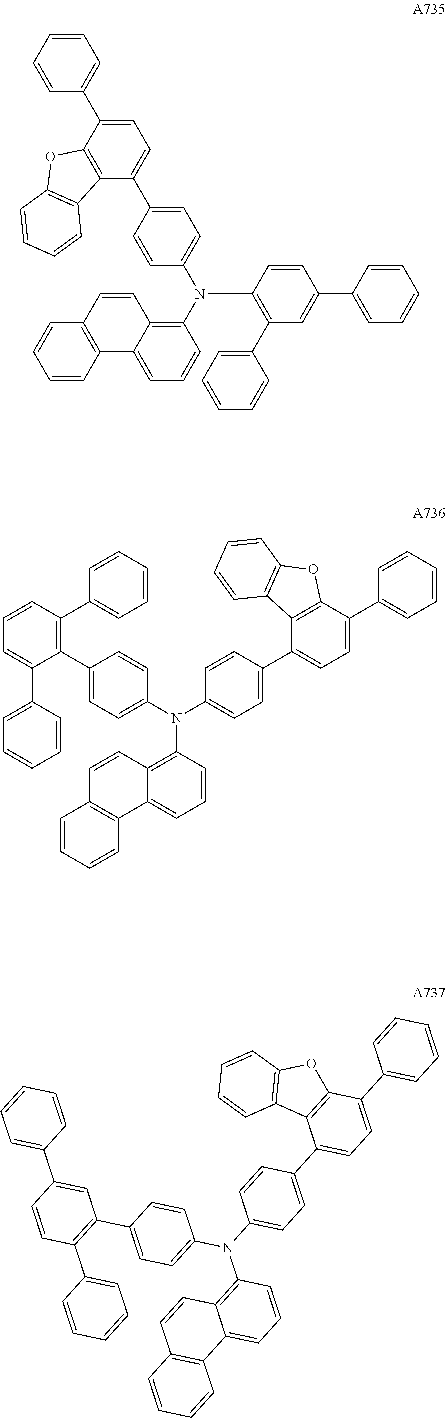 [SMILES: c1ccc(-c2ccc(N(c3ccc(-c4ccc(-c5ccccc5)c5oc6ccccc6c45)cc3)c3cccc4c3ccc3ccccc34)c(-c3ccccc3)c2)cc1 ; c1ccc(-c2cccc(-c3ccccc3)c2-c2ccc(N(c3ccc(-c4ccc(-c5ccccc5)c5oc6ccccc6c45)cc3)c3cccc4c3ccc3ccccc34)cc2)cc1 ; c1ccc(-c2ccc(-c3ccccc3)c(-c3ccc(N(c4ccc(-c5ccc(-c6ccccc6)c6oc7ccccc7c56)cc4)c4cccc5c4ccc4ccccc45)cc3)c2)cc1]

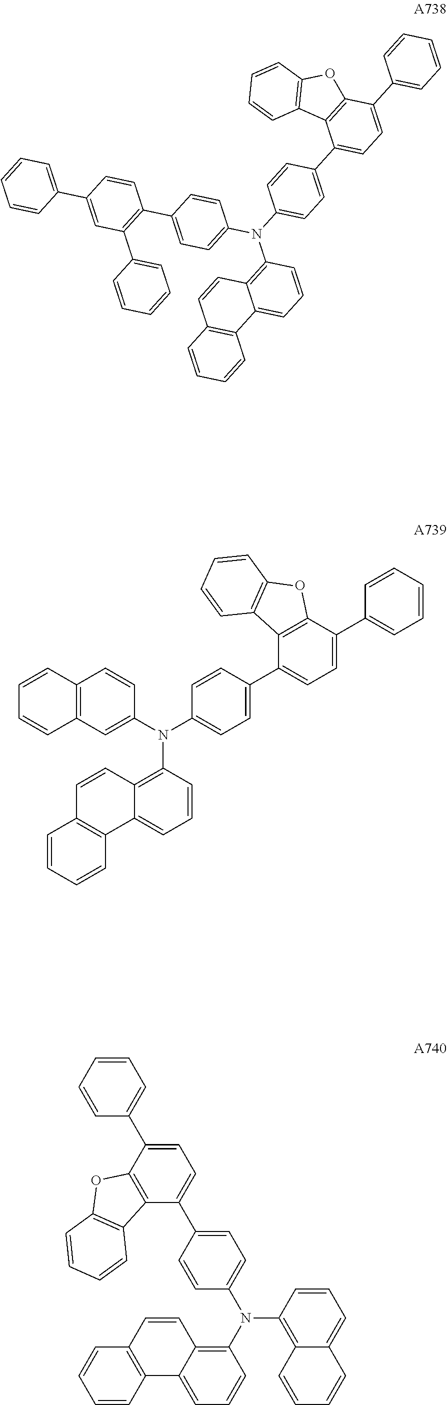 [SMILES: c1ccc(-c2ccc(-c3ccc(N(c4ccc(-c5ccc(-c6ccccc6)c6oc7ccccc7c56)cc4)c4cccc5c4ccc4ccccc45)cc3)c(-c3ccccc3)c2)cc1 ; c1ccc(-c2ccc(-c3ccc(N(c4ccc5ccccc5c4)c4cccc5c4ccc4ccccc45)cc3)c3c2oc2ccccc23)cc1 ; c1ccc(-c2ccc(-c3ccc(N(c4cccc5ccccc45)c4cccc5c4ccc4ccccc45)cc3)c3c2oc2ccccc23)cc1]

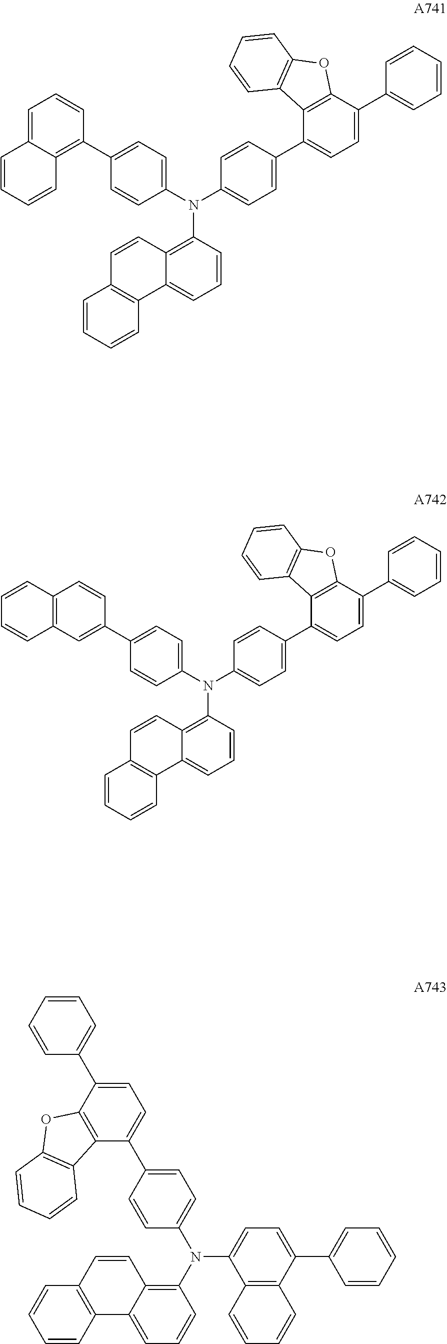 [SMILES: c1ccc(-c2ccc(-c3ccc(N(c4ccc(-c5cccc6ccccc56)cc4)c4cccc5c4ccc4ccccc45)cc3)c3c2oc2ccccc23)cc1 ; c1ccc(-c2ccc(-c3ccc(N(c4ccc(-c5ccc6ccccc6c5)cc4)c4cccc5c4ccc4ccccc45)cc3)c3c2oc2ccccc23)cc1 ; c1ccc(-c2ccc(N(c3ccc(-c4ccc(-c5ccccc5)c5oc6ccccc6c45)cc3)c3cccc4c3ccc3ccccc34)c3ccccc23)cc1]

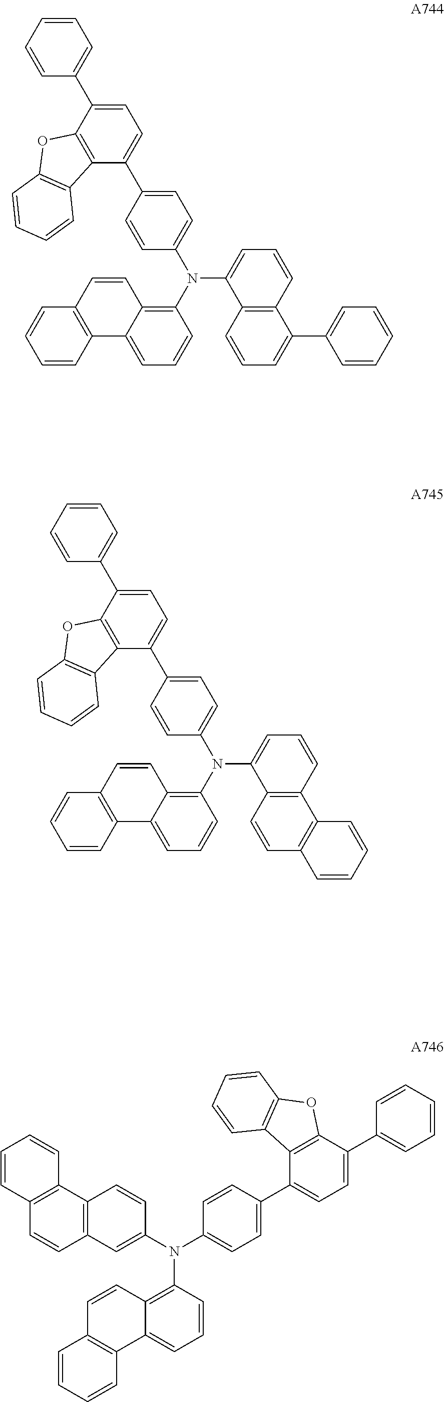 [SMILES: c1ccc(-c2cccc3c(N(c4ccc(-c5ccc(-c6ccccc6)c6oc7ccccc7c56)cc4)c4cccc5c4ccc4ccccc45)cccc23)cc1 ; c1ccc(-c2ccc(-c3ccc(N(c4cccc5c4ccc4ccccc45)c4cccc5c4ccc4ccccc45)cc3)c3c2oc2ccccc23)cc1 ; c1ccc(-c2ccc(-c3ccc(N(c4ccc5c(ccc6ccccc65)c4)c4cccc5c4ccc4ccccc45)cc3)c3c2oc2ccccc23)cc1]

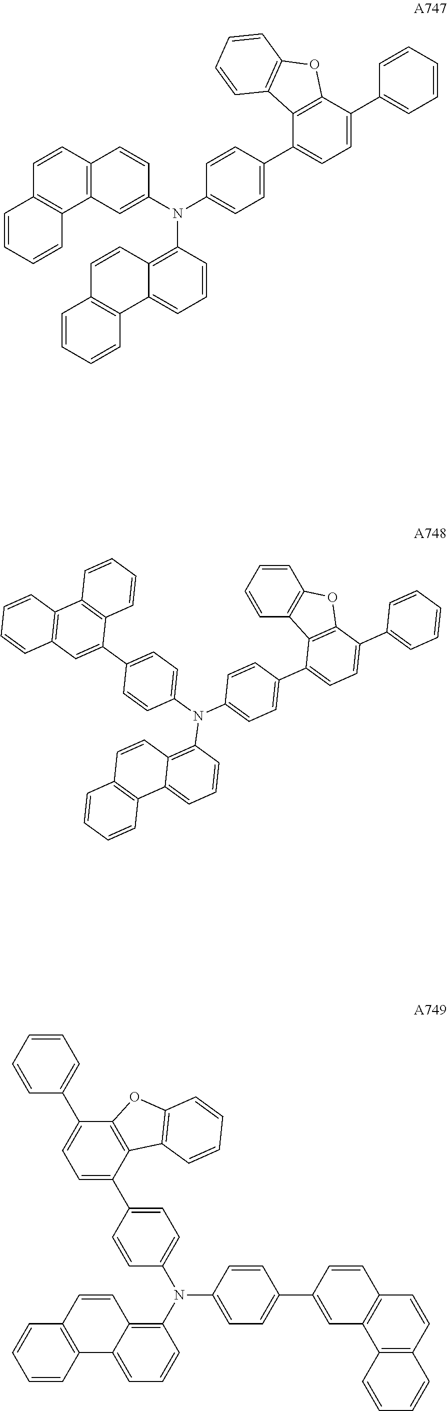 [SMILES: c1ccc(-c2ccc(-c3ccc(N(c4ccc5ccc6ccccc6c5c4)c4cccc5c4ccc4ccccc45)cc3)c3c2oc2ccccc23)cc1 ; c1ccc(-c2ccc(-c3ccc(N(c4ccc(-c5cc6ccccc6c6ccccc56)cc4)c4cccc5c4ccc4ccccc45)cc3)c3c2oc2ccccc23)cc1 ; c1ccc(-c2ccc(-c3ccc(N(c4ccc(-c5ccc6ccc7ccccc7c6c5)cc4)c4cccc5c4ccc4ccccc45)cc3)c3c2oc2ccccc23)cc1]

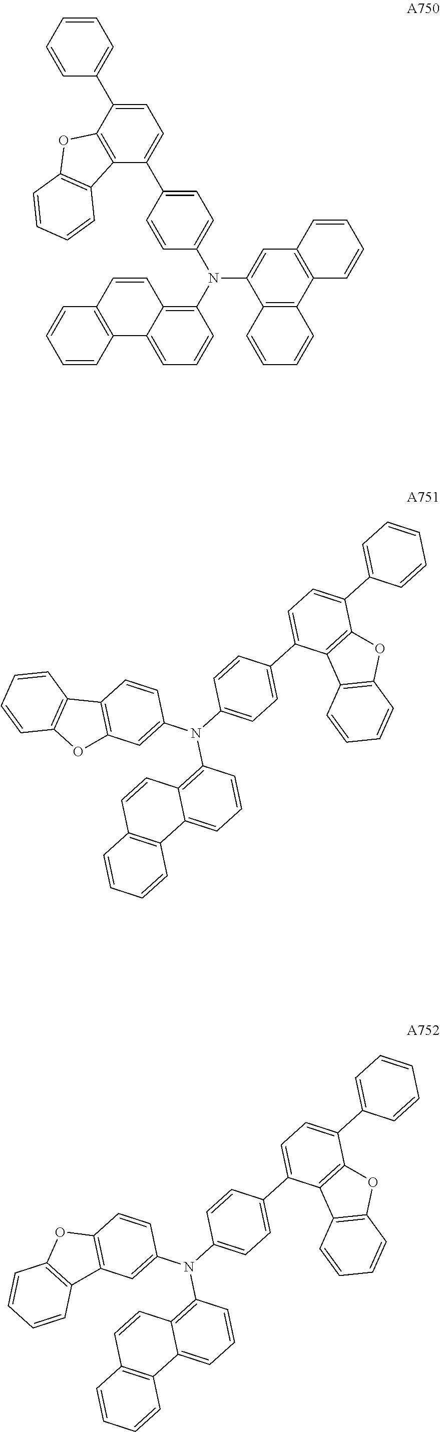 [SMILES: c1ccc(-c2ccc(-c3ccc(N(c4cccc5c4ccc4ccccc45)c4cc5ccccc5c5ccccc45)cc3)c3c2oc2ccccc23)cc1 ; c1ccc(-c2ccc(-c3ccc(N(c4ccc5c(c4)oc4ccccc45)c4cccc5c4ccc4ccccc45)cc3)c3c2oc2ccccc23)cc1 ; c1ccc(-c2ccc(-c3ccc(N(c4ccc5oc6ccccc6c5c4)c4cccc5c4ccc4ccccc45)cc3)c3c2oc2ccccc23)cc1]

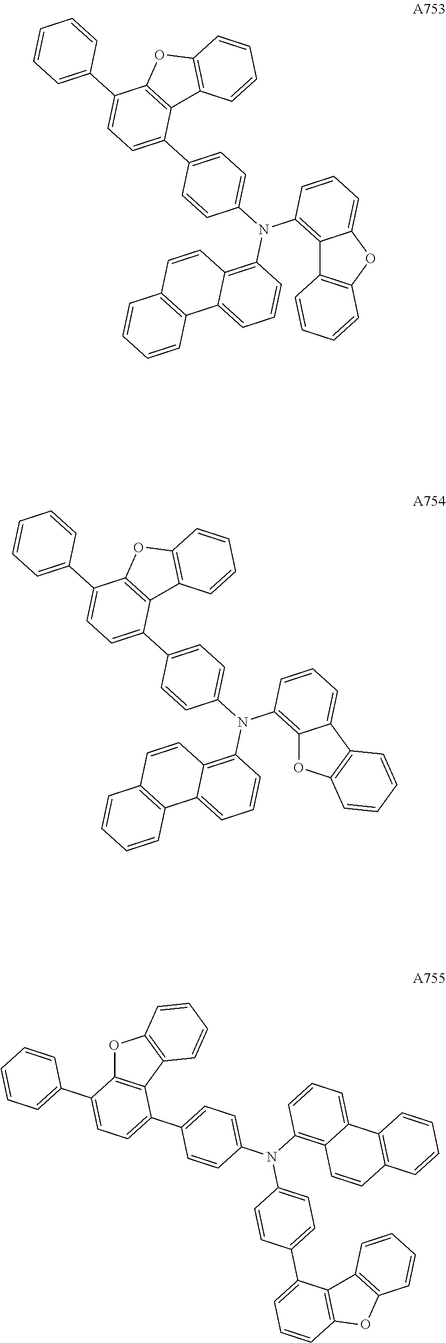 [SMILES: c1ccc(-c2ccc(-c3ccc(N(c4cccc5c4ccc4ccccc45)c4cccc5oc6ccccc6c45)cc3)c3c2oc2ccccc23)cc1 ; c1ccc(-c2ccc(-c3ccc(N(c4cccc5c4ccc4ccccc45)c4cccc5c4oc4ccccc45)cc3)c3c2oc2ccccc23)cc1 ; c1ccc(-c2ccc(-c3ccc(N(c4ccc(-c5cccc6oc7ccccc7c56)cc4)c4cccc5c4ccc4ccccc45)cc3)c3c2oc2ccccc23)cc1]

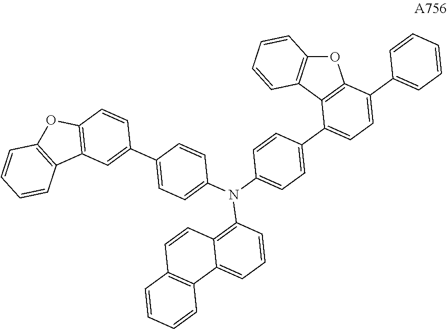 [SMILES: c1ccc(-c2ccc(-c3ccc(N(c4ccc(-c5ccc6oc7ccccc7c6c5)cc4)c4cccc5c4ccc4ccccc45)cc3)c3c2oc2ccccc23)cc1]

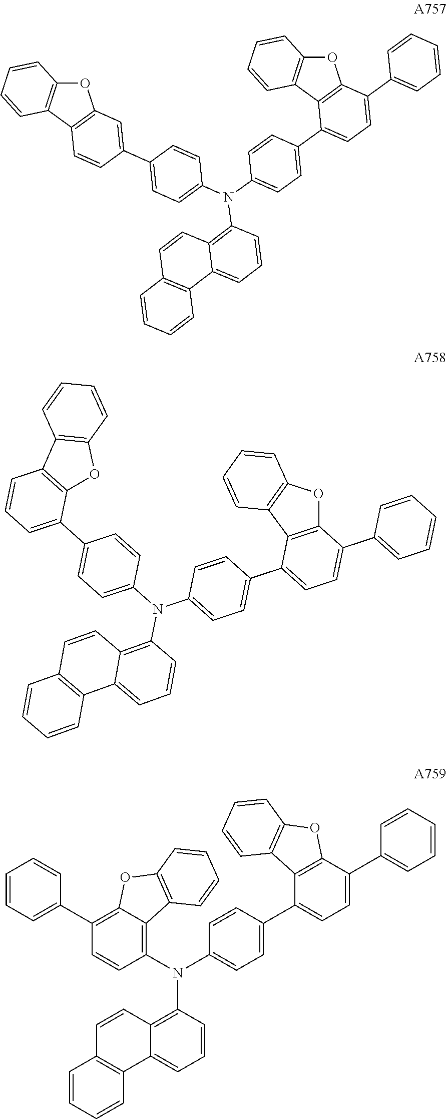 [SMILES: c1ccc(-c2ccc(-c3ccc(N(c4ccc(-c5ccc6c(c5)oc5ccccc56)cc4)c4cccc5c4ccc4ccccc45)cc3)c3c2oc2ccccc23)cc1 ; c1ccc(-c2ccc(-c3ccc(N(c4ccc(-c5cccc6c5oc5ccccc56)cc4)c4cccc5c4ccc4ccccc45)cc3)c3c2oc2ccccc23)cc1 ; c1ccc(-c2ccc(-c3ccc(N(c4cccc5c4ccc4ccccc45)c4ccc(-c5ccccc5)c5oc6ccccc6c45)cc3)c3c2oc2ccccc23)cc1]

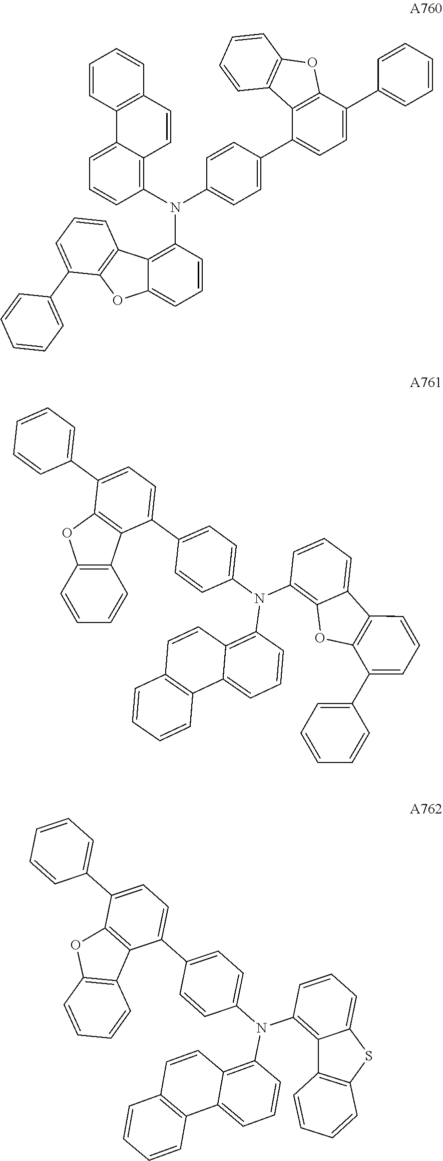 [SMILES: c1ccc(-c2cccc3c2oc2cccc(N(c4ccc(-c5ccc(-c6ccccc6)c6oc7ccccc7c56)cc4)c4cccc5c4ccc4ccccc45)c23)cc1 ; c1ccc(-c2cccc3c2oc2c(N(c4ccc(-c5ccc(-c6ccccc6)c6oc7ccccc7c56)cc4)c4cccc5c4ccc4ccccc45)cccc23)cc1 ; c1ccc(-c2ccc(-c3ccc(N(c4cccc5c4ccc4ccccc45)c4cccc5sc6ccccc6c45)cc3)c3c2oc2ccccc23)cc1]

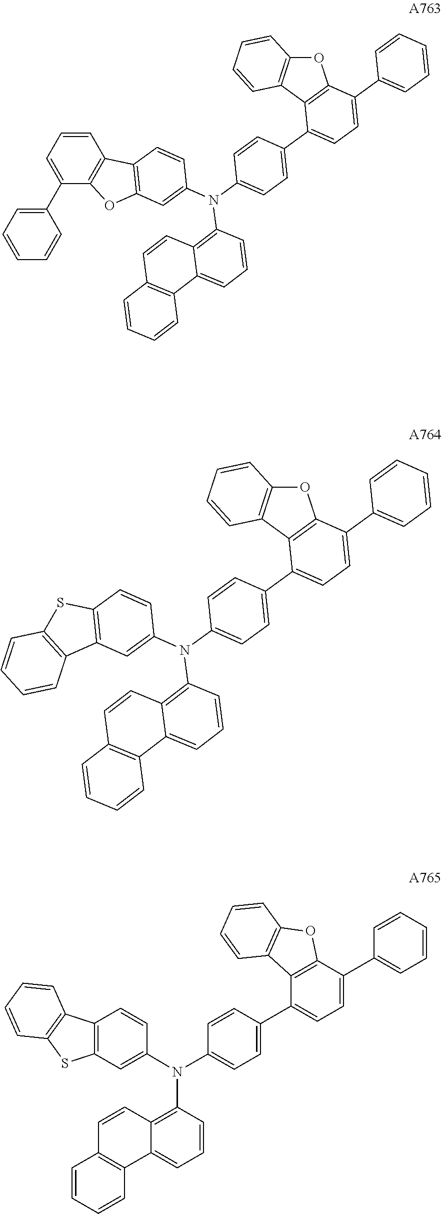 [SMILES: c1ccc(-c2cccc3c2oc2cc(N(c4ccc(-c5ccc(-c6ccccc6)c6oc7ccccc7c56)cc4)c4cccc5c4ccc4ccccc45)ccc23)cc1 ; c1ccc(-c2ccc(-c3ccc(N(c4ccc5sc6ccccc6c5c4)c4cccc5c4ccc4ccccc45)cc3)c3c2oc2ccccc23)cc1 ; c1ccc(-c2ccc(-c3ccc(N(c4ccc5c(c4)sc4ccccc45)c4cccc5c4ccc4ccccc45)cc3)c3c2oc2ccccc23)cc1]

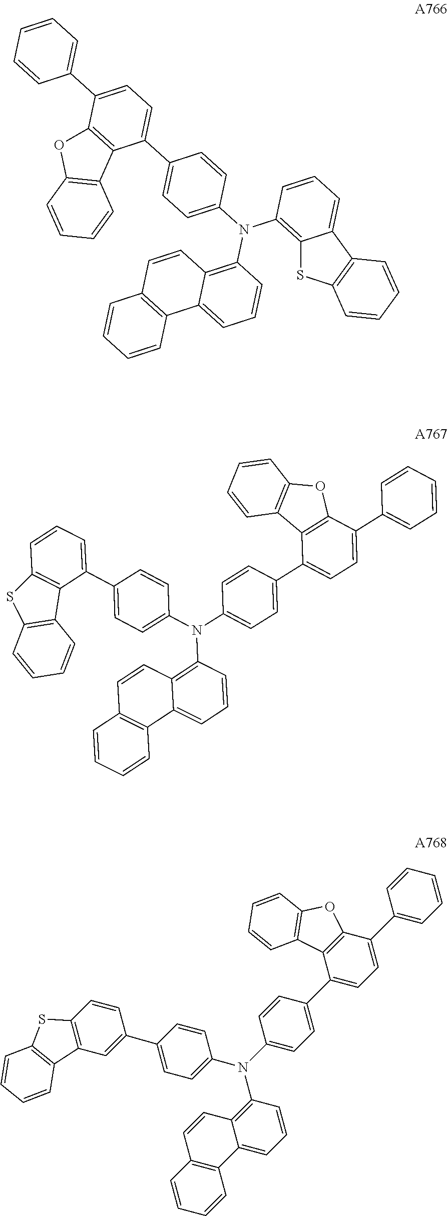 [SMILES: c1ccc(-c2ccc(-c3ccc(N(c4cccc5c4ccc4ccccc45)c4cccc5c4sc4ccccc45)cc3)c3c2oc2ccccc23)cc1 ; c1ccc(-c2ccc(-c3ccc(N(c4ccc(-c5cccc6sc7ccccc7c56)cc4)c4cccc5c4ccc4ccccc45)cc3)c3c2oc2ccccc23)cc1 ; c1ccc(-c2ccc(-c3ccc(N(c4ccc(-c5ccc6sc7ccccc7c6c5)cc4)c4cccc5c4ccc4ccccc45)cc3)c3c2oc2ccccc23)cc1]

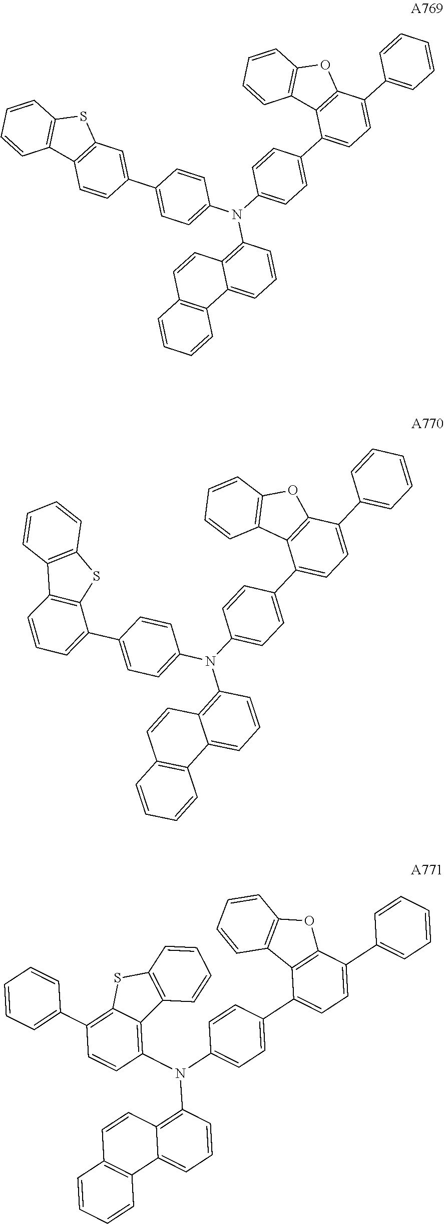 [SMILES: c1ccc(-c2ccc(-c3ccc(N(c4ccc(-c5ccc6c(c5)sc5ccccc56)cc4)c4cccc5c4ccc4ccccc45)cc3)c3c2oc2ccccc23)cc1 ; c1ccc(-c2ccc(-c3ccc(N(c4ccc(-c5cccc6c5sc5ccccc56)cc4)c4cccc5c4ccc4ccccc45)cc3)c3c2oc2ccccc23)cc1 ; c1ccc(-c2ccc(-c3ccc(N(c4cccc5c4ccc4ccccc45)c4ccc(-c5ccccc5)c5sc6ccccc6c45)cc3)c3c2oc2ccccc23)cc1]

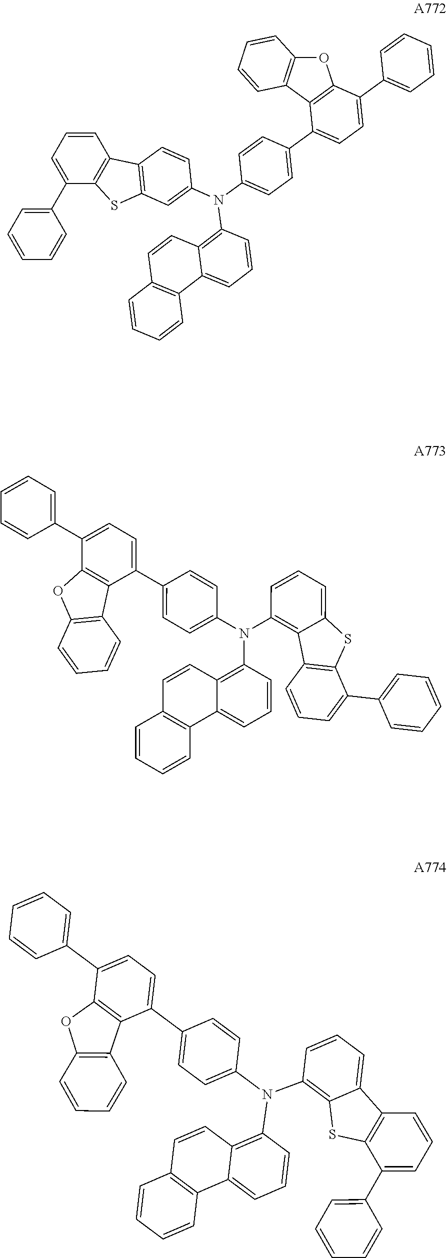 [SMILES: c1ccc(-c2ccc(-c3ccc(N(c4ccc5c(c4)sc4c(-c6ccccc6)cccc45)c4cccc5c4ccc4ccccc45)cc3)c3c2oc2ccccc23)cc1 ; c1ccc(-c2ccc(-c3ccc(N(c4cccc5c4ccc4ccccc45)c4cccc5sc6c(-c7ccccc7)cccc6c45)cc3)c3c2oc2ccccc23)cc1 ; c1ccc(-c2ccc(-c3ccc(N(c4cccc5c4ccc4ccccc45)c4cccc5c4sc4c(-c6ccccc6)cccc45)cc3)c3c2oc2ccccc23)cc1]

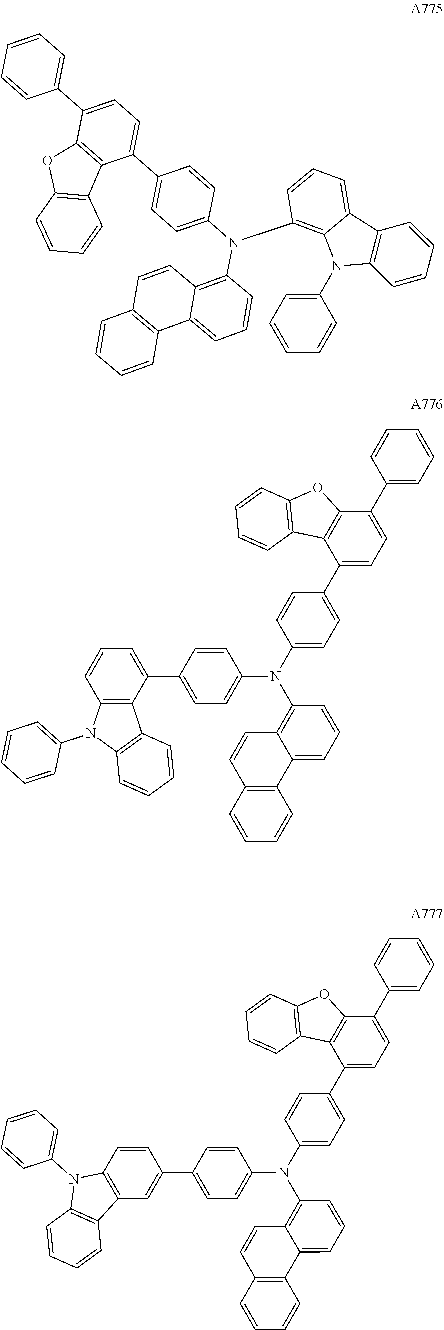 [SMILES: c1ccc(-c2ccc(-c3ccc(N(c4cccc5c4ccc4ccccc45)c4cccc5c6ccccc6n(-c6ccccc6)c45)cc3)c3c2oc2ccccc23)cc1 ; c1ccc(-c2ccc(-c3ccc(N(c4ccc(-c5cccc6c5c5ccccc5n6-c5ccccc5)cc4)c4cccc5c4ccc4ccccc45)cc3)c3c2oc2ccccc23)cc1 ; c1ccc(-c2ccc(-c3ccc(N(c4ccc(-c5ccc6c(c5)c5ccccc5n6-c5ccccc5)cc4)c4cccc5c4ccc4ccccc45)cc3)c3c2oc2ccccc23)cc1]

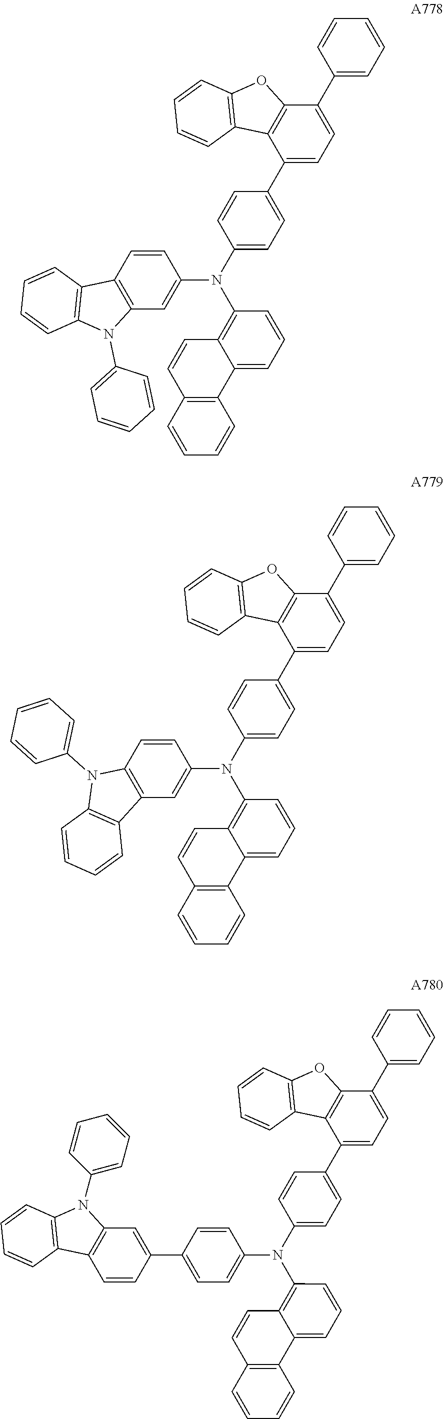 [SMILES: c1ccc(-c2ccc(-c3ccc(N(c4ccc5c6ccccc6n(-c6ccccc6)c5c4)c4cccc5c4ccc4ccccc45)cc3)c3c2oc2ccccc23)cc1 ; c1ccc(-c2ccc(-c3ccc(N(c4ccc5c(c4)c4ccccc4n5-c4ccccc4)c4cccc5c4ccc4ccccc45)cc3)c3c2oc2ccccc23)cc1 ; c1ccc(-c2ccc(-c3ccc(N(c4ccc(-c5ccc6c7ccccc7n(-c7ccccc7)c6c5)cc4)c4cccc5c4ccc4ccccc45)cc3)c3c2oc2ccccc23)cc1]

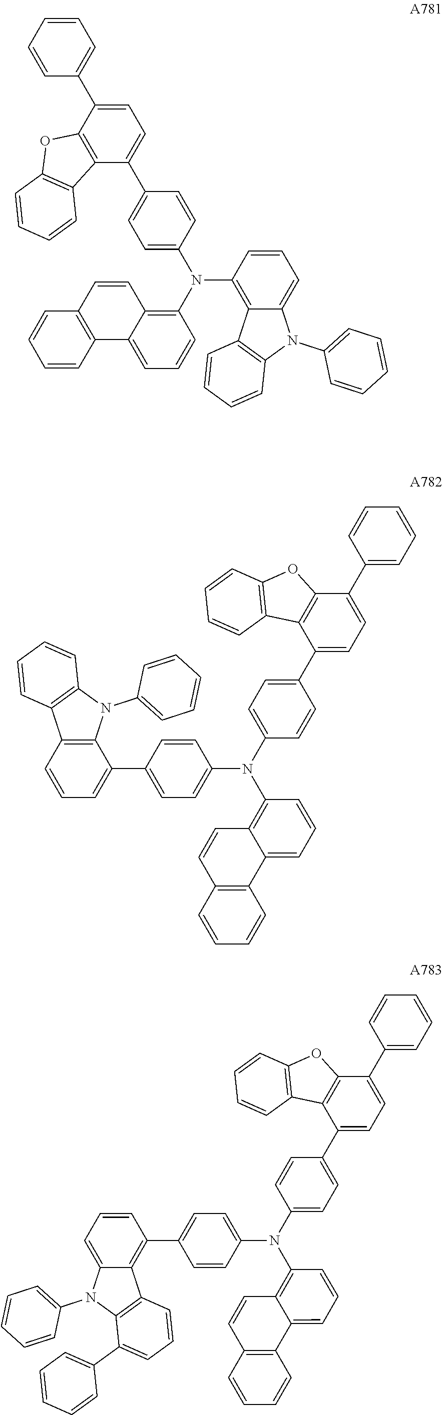 [SMILES: c1ccc(-c2ccc(-c3ccc(N(c4cccc5c4ccc4ccccc45)c4cccc5c4c4ccccc4n5-c4ccccc4)cc3)c3c2oc2ccccc23)cc1 ; c1ccc(-c2ccc(-c3ccc(N(c4ccc(-c5cccc6c7ccccc7n(-c7ccccc7)c56)cc4)c4cccc5c4ccc4ccccc45)cc3)c3c2oc2ccccc23)cc1 ; c1ccc(-c2ccc(-c3ccc(N(c4ccc(-c5cccc6c5c5cccc(-c7ccccc7)c5n6-c5ccccc5)cc4)c4cccc5c4ccc4ccccc45)cc3)c3c2oc2ccccc23)cc1]

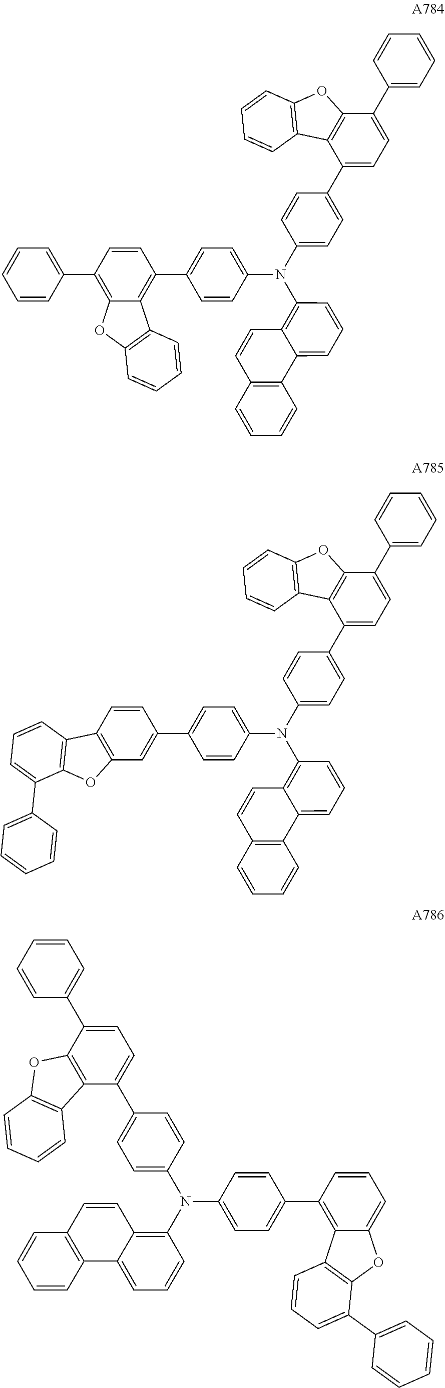 [SMILES: c1ccc(-c2ccc(-c3ccc(N(c4ccc(-c5ccc(-c6ccccc6)c6oc7ccccc7c56)cc4)c4cccc5c4ccc4ccccc45)cc3)c3c2oc2ccccc23)cc1 ; c1ccc(-c2cccc3c2oc2cc(-c4ccc(N(c5ccc(-c6ccc(-c7ccccc7)c7oc8ccccc8c67)cc5)c5cccc6c5ccc5ccccc56)cc4)ccc23)cc1 ; c1ccc(-c2cccc3c2oc2cccc(-c4ccc(N(c5ccc(-c6ccc(-c7ccccc7)c7oc8ccccc8c67)cc5)c5cccc6c5ccc5ccccc56)cc4)c23)cc1]

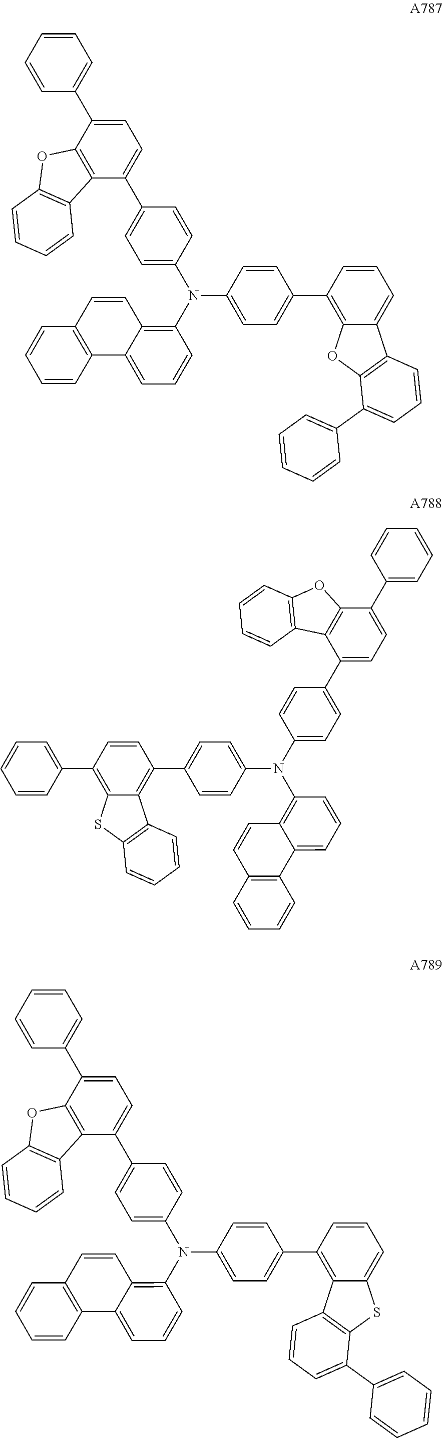 [SMILES: c1ccc(-c2cccc3c2oc2c(-c4ccc(N(c5ccc(-c6ccc(-c7ccccc7)c7oc8ccccc8c67)cc5)c5cccc6c5ccc5ccccc56)cc4)cccc23)cc1 ; c1ccc(-c2ccc(-c3ccc(N(c4ccc(-c5ccc(-c6ccccc6)c6sc7ccccc7c56)cc4)c4cccc5c4ccc4ccccc45)cc3)c3c2oc2ccccc23)cc1 ; c1ccc(-c2ccc(-c3ccc(N(c4ccc(-c5cccc6sc7c(-c8ccccc8)cccc7c56)cc4)c4cccc5c4ccc4ccccc45)cc3)c3c2oc2ccccc23)cc1]

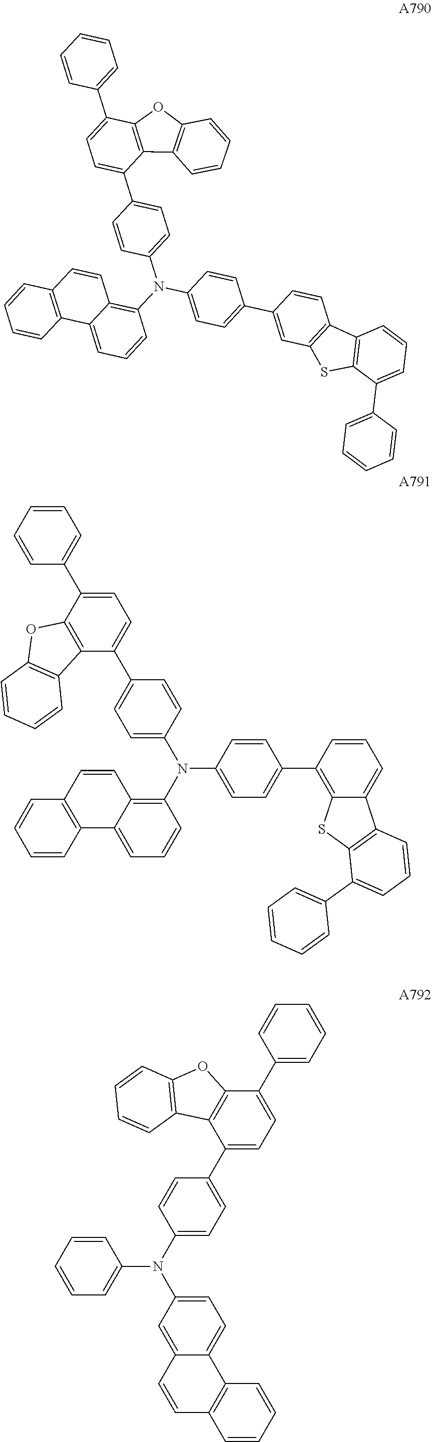 [SMILES: c1ccc(-c2ccc(-c3ccc(N(c4ccc(-c5ccc6c(c5)sc5c(-c7ccccc7)cccc56)cc4)c4cccc5c4ccc4ccccc45)cc3)c3c2oc2ccccc23)cc1 ; c1ccc(-c2ccc(-c3ccc(N(c4ccc(-c5cccc6c5sc5c(-c7ccccc7)cccc56)cc4)c4cccc5c4ccc4ccccc45)cc3)c3c2oc2ccccc23)cc1 ; c1ccc(-c2ccc(-c3ccc(N(c4ccccc4)c4ccc5c(ccc6ccccc65)c4)cc3)c3c2oc2ccccc23)cc1]

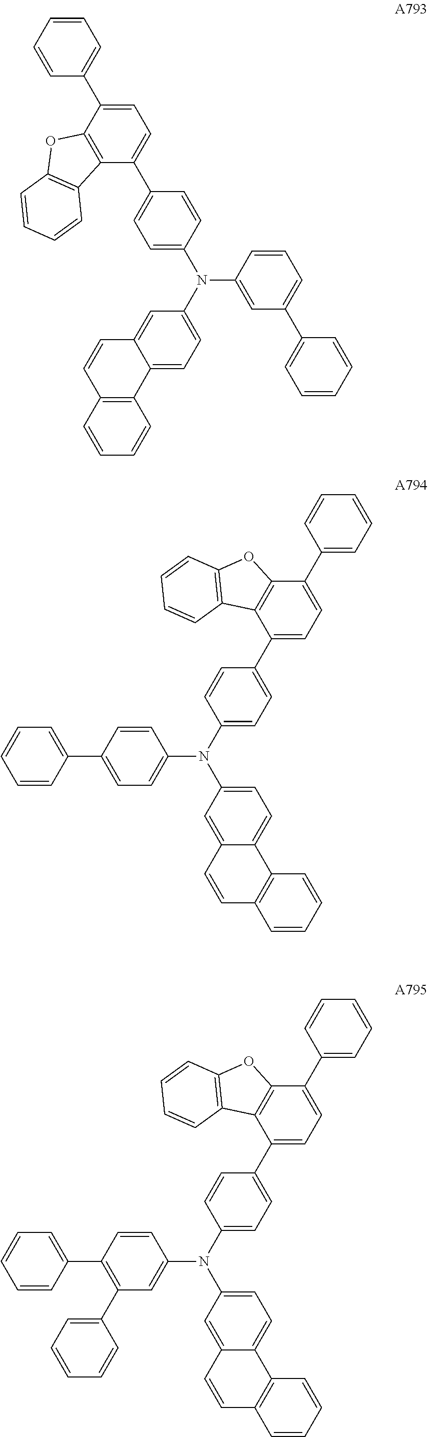 [SMILES: c1ccc(-c2cccc(N(c3ccc(-c4ccc(-c5ccccc5)c5oc6ccccc6c45)cc3)c3ccc4c(ccc5ccccc54)c3)c2)cc1 ; c1ccc(-c2ccc(N(c3ccc(-c4ccc(-c5ccccc5)c5oc6ccccc6c45)cc3)c3ccc4c(ccc5ccccc54)c3)cc2)cc1 ; c1ccc(-c2ccc(N(c3ccc(-c4ccc(-c5ccccc5)c5oc6ccccc6c45)cc3)c3ccc4c(ccc5ccccc54)c3)cc2-c2ccccc2)cc1]

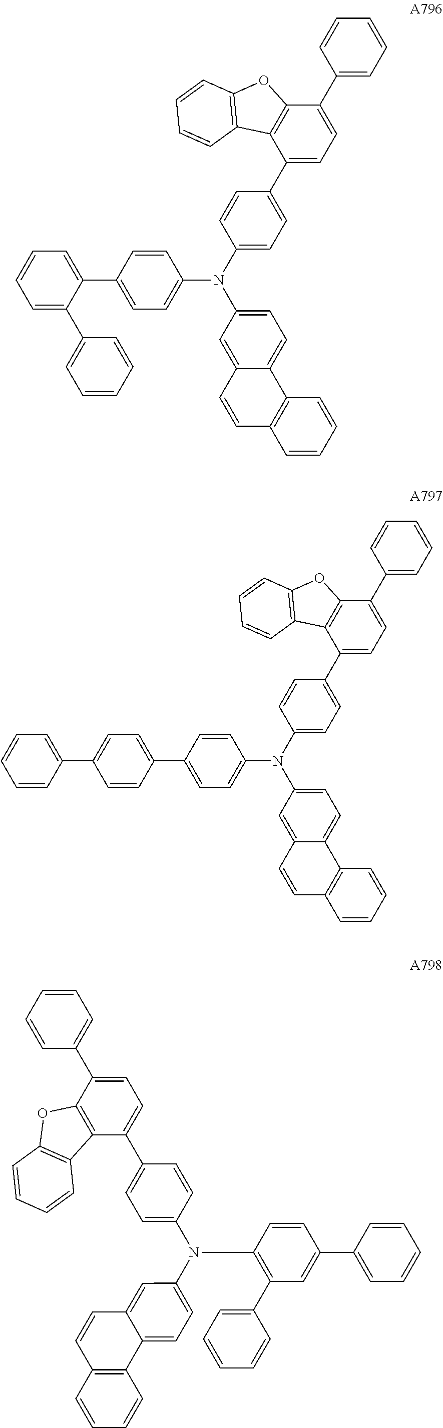 [SMILES: c1ccc(-c2ccccc2-c2ccc(N(c3ccc(-c4ccc(-c5ccccc5)c5oc6ccccc6c45)cc3)c3ccc4c(ccc5ccccc54)c3)cc2)cc1 ; c1ccc(-c2ccc(-c3ccc(N(c4ccc(-c5ccc(-c6ccccc6)c6oc7ccccc7c56)cc4)c4ccc5c(ccc6ccccc65)c4)cc3)cc2)cc1 ; c1ccc(-c2ccc(N(c3ccc(-c4ccc(-c5ccccc5)c5oc6ccccc6c45)cc3)c3ccc4c(ccc5ccccc54)c3)c(-c3ccccc3)c2)cc1]

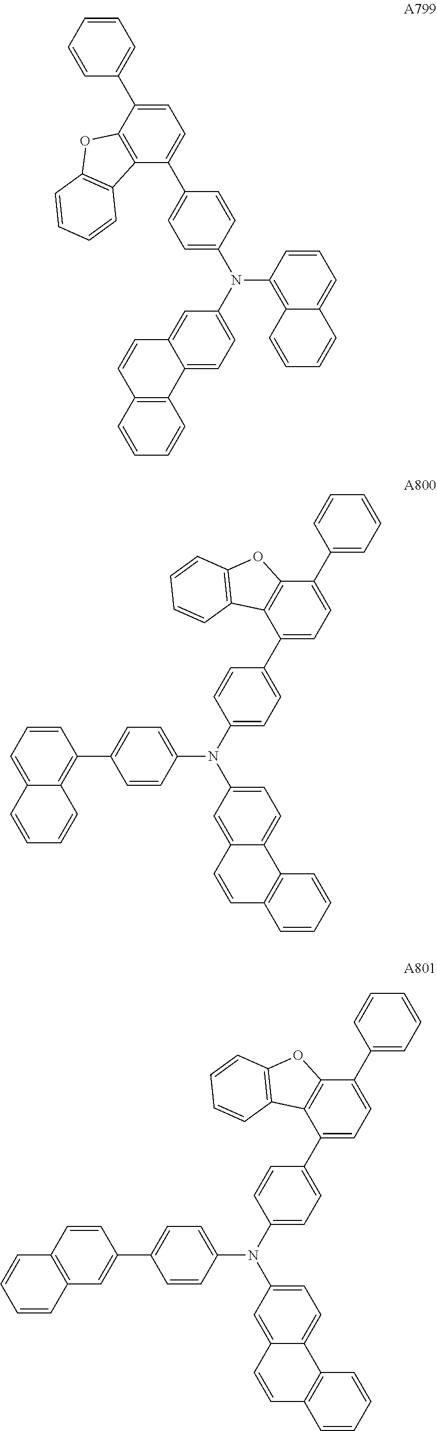 [SMILES: c1ccc(-c2ccc(-c3ccc(N(c4ccc5c(ccc6ccccc65)c4)c4cccc5ccccc45)cc3)c3c2oc2ccccc23)cc1 ; c1ccc(-c2ccc(-c3ccc(N(c4ccc(-c5cccc6ccccc56)cc4)c4ccc5c(ccc6ccccc65)c4)cc3)c3c2oc2ccccc23)cc1 ; c1ccc(-c2ccc(-c3ccc(N(c4ccc(-c5ccc6ccccc6c5)cc4)c4ccc5c(ccc6ccccc65)c4)cc3)c3c2oc2ccccc23)cc1]

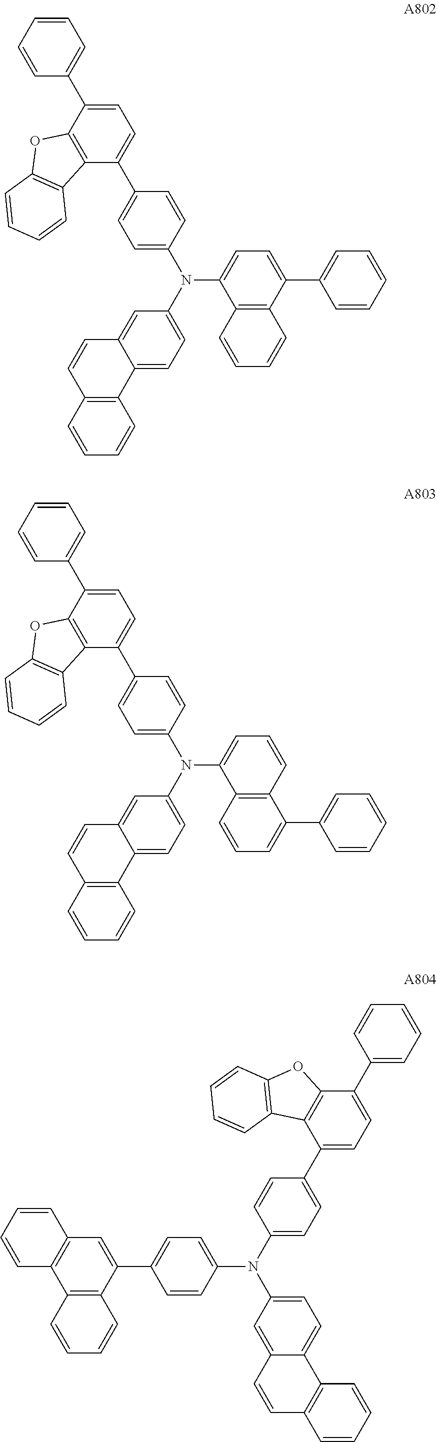 [SMILES: c1ccc(-c2ccc(N(c3ccc(-c4ccc(-c5ccccc5)c5oc6ccccc6c45)cc3)c3ccc4c(ccc5ccccc54)c3)c3ccccc23)cc1 ; c1ccc(-c2cccc3c(N(c4ccc(-c5ccc(-c6ccccc6)c6oc7ccccc7c56)cc4)c4ccc5c(ccc6ccccc65)c4)cccc23)cc1 ; c1ccc(-c2ccc(-c3ccc(N(c4ccc(-c5cc6ccccc6c6ccccc56)cc4)c4ccc5c(ccc6ccccc65)c4)cc3)c3c2oc2ccccc23)cc1]

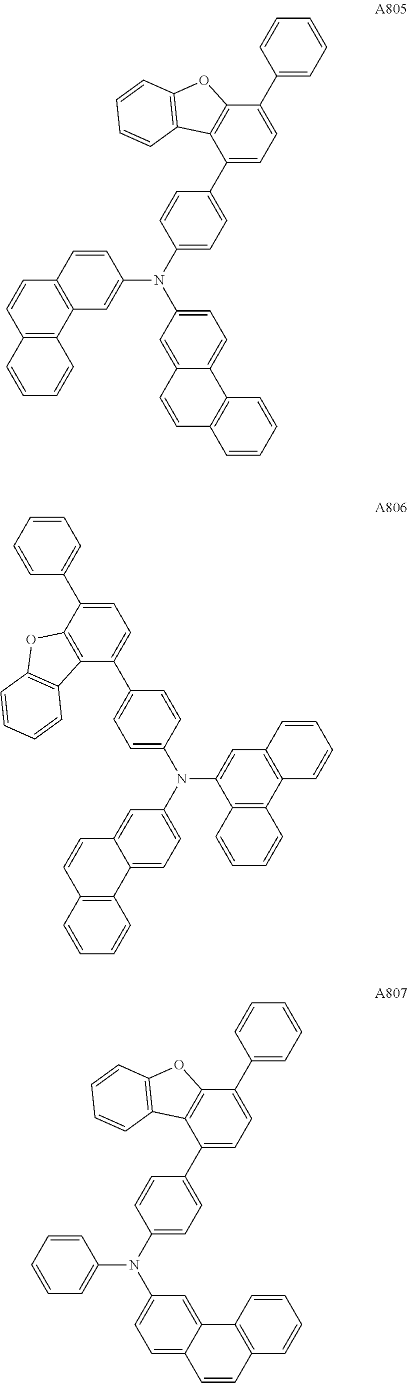 [SMILES: c1ccc(-c2ccc(-c3ccc(N(c4ccc5c(ccc6ccccc65)c4)c4ccc5ccc6ccccc6c5c4)cc3)c3c2oc2ccccc23)cc1 ; c1ccc(-c2ccc(-c3ccc(N(c4ccc5c(ccc6ccccc65)c4)c4cc5ccccc5c5ccccc45)cc3)c3c2oc2ccccc23)cc1 ; c1ccc(-c2ccc(-c3ccc(N(c4ccccc4)c4ccc5ccc6ccccc6c5c4)cc3)c3c2oc2ccccc23)cc1]

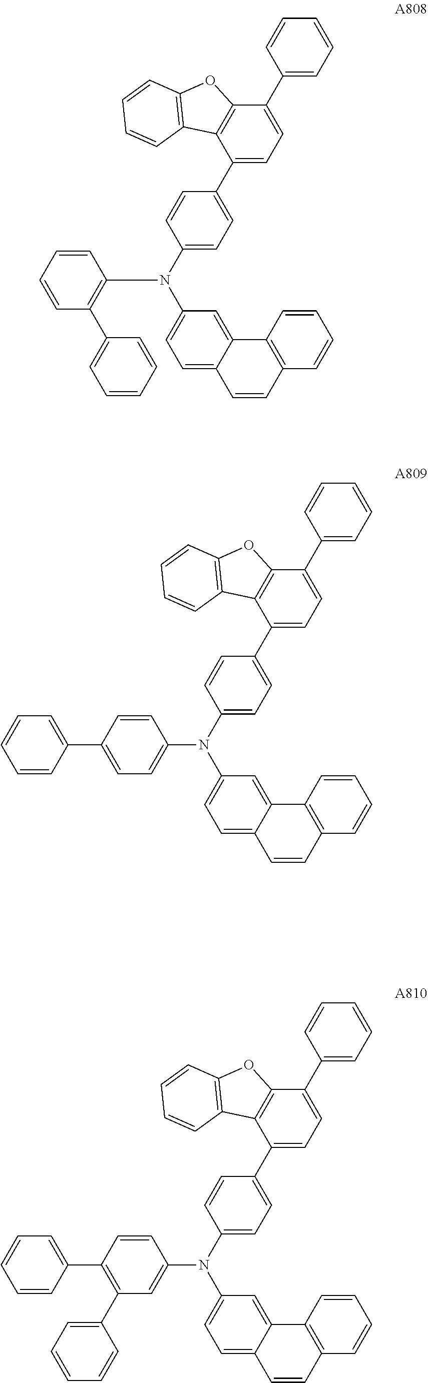 [SMILES: c1ccc(-c2ccccc2N(c2ccc(-c3ccc(-c4ccccc4)c4oc5ccccc5c34)cc2)c2ccc3ccc4ccccc4c3c2)cc1 ; c1ccc(-c2ccc(N(c3ccc(-c4ccc(-c5ccccc5)c5oc6ccccc6c45)cc3)c3ccc4ccc5ccccc5c4c3)cc2)cc1 ; c1ccc(-c2ccc(N(c3ccc(-c4ccc(-c5ccccc5)c5oc6ccccc6c45)cc3)c3ccc4ccc5ccccc5c4c3)cc2-c2ccccc2)cc1]

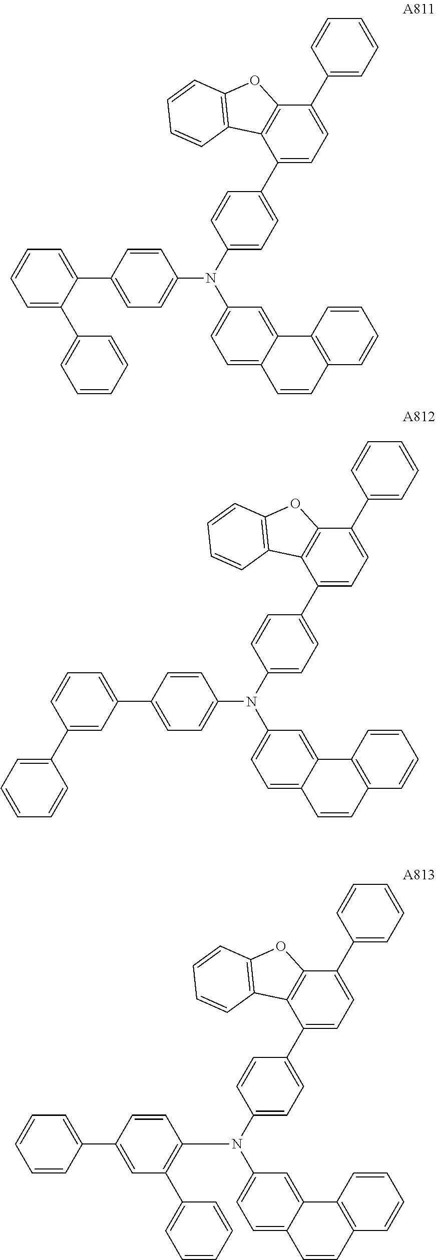 [SMILES: c1ccc(-c2ccccc2-c2ccc(N(c3ccc(-c4ccc(-c5ccccc5)c5oc6ccccc6c45)cc3)c3ccc4ccc5ccccc5c4c3)cc2)cc1 ; c1ccc(-c2cccc(-c3ccc(N(c4ccc(-c5ccc(-c6ccccc6)c6oc7ccccc7c56)cc4)c4ccc5ccc6ccccc6c5c4)cc3)c2)cc1 ; c1ccc(-c2ccc(N(c3ccc(-c4ccc(-c5ccccc5)c5oc6ccccc6c45)cc3)c3ccc4ccc5ccccc5c4c3)c(-c3ccccc3)c2)cc1]

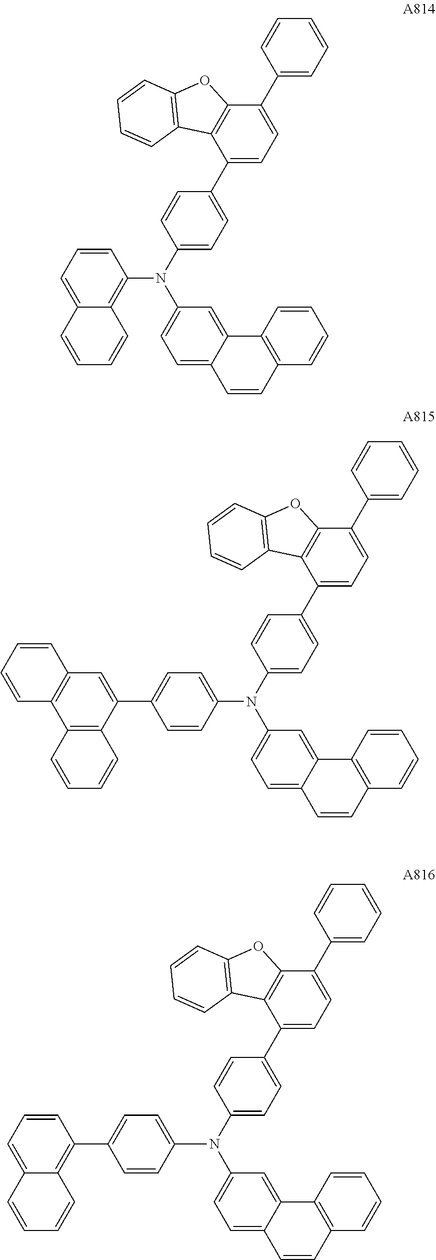 [SMILES: c1ccc(-c2ccc(-c3ccc(N(c4ccc5ccc6ccccc6c5c4)c4cccc5ccccc45)cc3)c3c2oc2ccccc23)cc1 ; c1ccc(-c2ccc(-c3ccc(N(c4ccc(-c5cc6ccccc6c6ccccc56)cc4)c4ccc5ccc6ccccc6c5c4)cc3)c3c2oc2ccccc23)cc1 ; c1ccc(-c2ccc(-c3ccc(N(c4ccc(-c5cccc6ccccc56)cc4)c4ccc5ccc6ccccc6c5c4)cc3)c3c2oc2ccccc23)cc1]

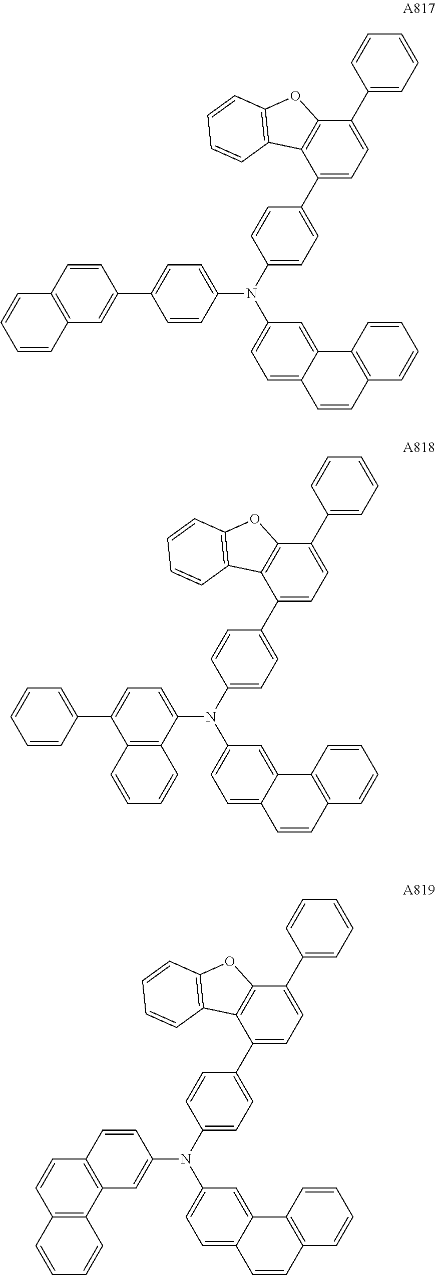 [SMILES: c1ccc(-c2ccc(-c3ccc(N(c4ccc(-c5ccc6ccccc6c5)cc4)c4ccc5ccc6ccccc6c5c4)cc3)c3c2oc2ccccc23)cc1 ; c1ccc(-c2ccc(N(c3ccc(-c4ccc(-c5ccccc5)c5oc6ccccc6c45)cc3)c3ccc4ccc5ccccc5c4c3)c3ccccc23)cc1 ; c1ccc(-c2ccc(-c3ccc(N(c4ccc5ccc6ccccc6c5c4)c4ccc5ccc6ccccc6c5c4)cc3)c3c2oc2ccccc23)cc1]

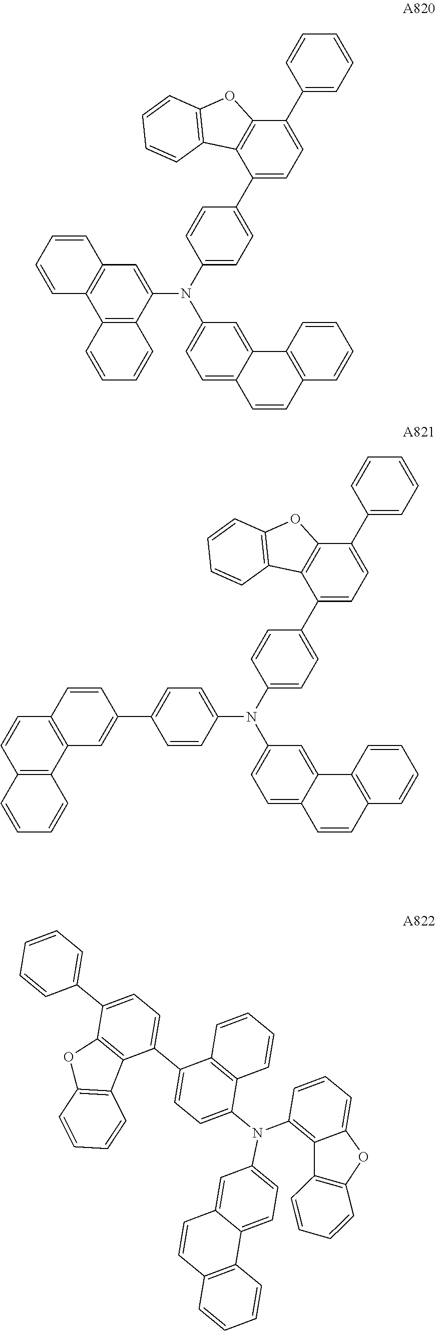 [SMILES: c1ccc(-c2ccc(-c3ccc(N(c4ccc5ccc6ccccc6c5c4)c4cc5ccccc5c5ccccc45)cc3)c3c2oc2ccccc23)cc1 ; c1ccc(-c2ccc(-c3ccc(N(c4ccc(-c5ccc6ccc7ccccc7c6c5)cc4)c4ccc5ccc6ccccc6c5c4)cc3)c3c2oc2ccccc23)cc1 ; c1ccc(-c2ccc(-c3ccc(N(c4ccc5c(ccc6ccccc65)c4)c4cccc5oc6ccccc6c45)c4ccccc34)c3c2oc2ccccc23)cc1]

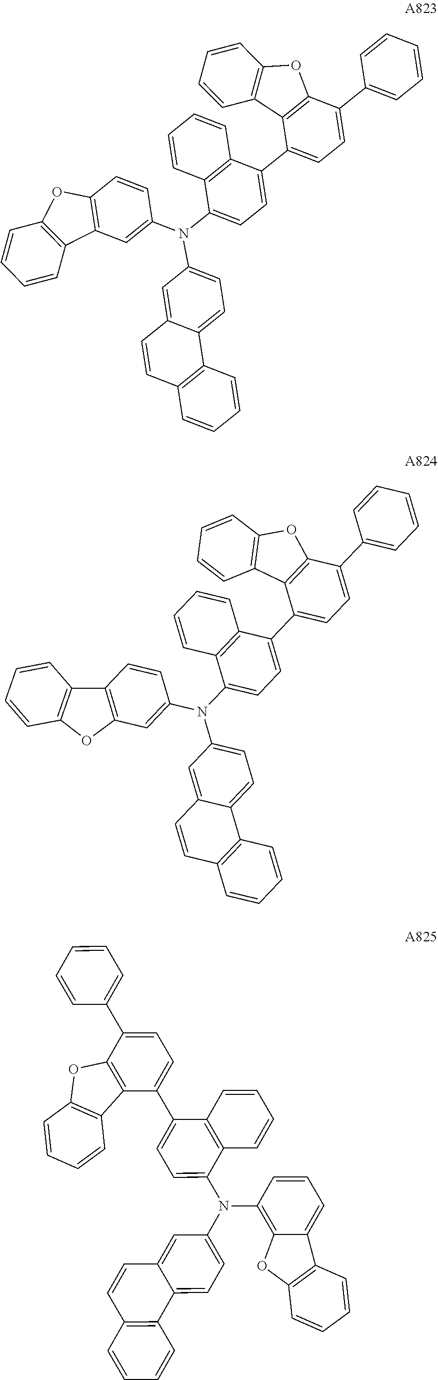 [SMILES: c1ccc(-c2ccc(-c3ccc(N(c4ccc5c(ccc6ccccc65)c4)c4ccc5oc6ccccc6c5c4)c4ccccc34)c3c2oc2ccccc23)cc1 ; c1ccc(-c2ccc(-c3ccc(N(c4ccc5c(ccc6ccccc65)c4)c4ccc5c(c4)oc4ccccc45)c4ccccc34)c3c2oc2ccccc23)cc1 ; c1ccc(-c2ccc(-c3ccc(N(c4ccc5c(ccc6ccccc65)c4)c4cccc5c4oc4ccccc45)c4ccccc34)c3c2oc2ccccc23)cc1]

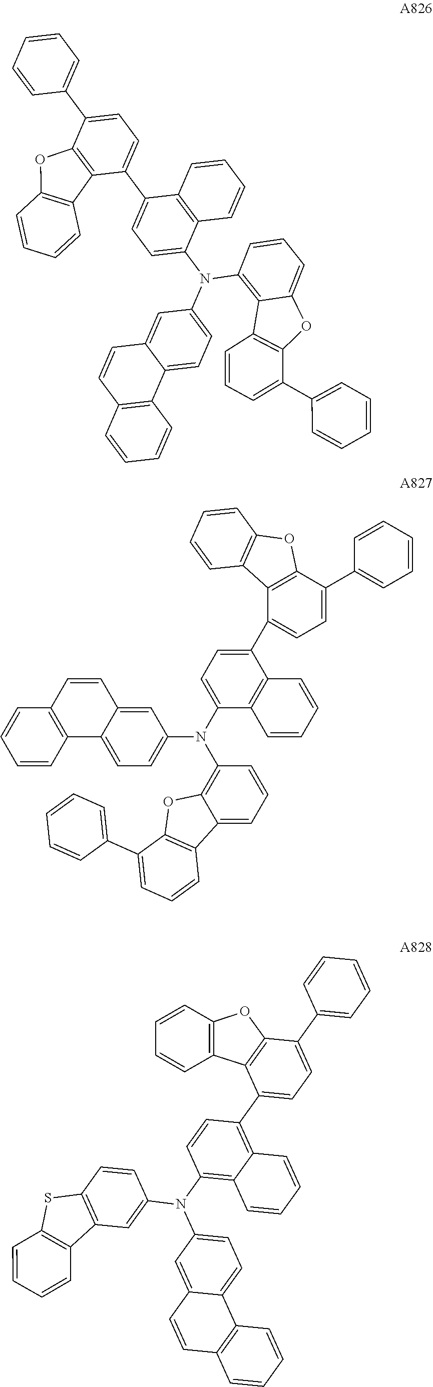 [SMILES: c1ccc(-c2cccc3c2oc2cccc(N(c4ccc5c(ccc6ccccc65)c4)c4ccc(-c5ccc(-c6ccccc6)c6oc7ccccc7c56)c5ccccc45)c23)cc1 ; c1ccc(-c2cccc3c2oc2c(N(c4ccc5c(ccc6ccccc65)c4)c4ccc(-c5ccc(-c6ccccc6)c6oc7ccccc7c56)c5ccccc45)cccc23)cc1 ; c1ccc(-c2ccc(-c3ccc(N(c4ccc5c(ccc6ccccc65)c4)c4ccc5sc6ccccc6c5c4)c4ccccc34)c3c2oc2ccccc23)cc1]

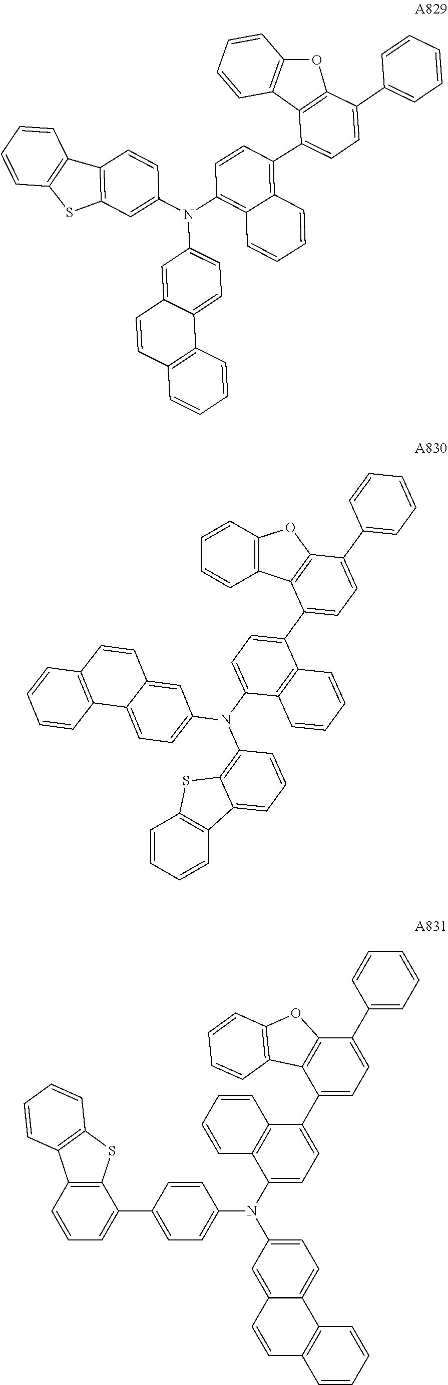 [SMILES: c1ccc(-c2ccc(-c3ccc(N(c4ccc5c(ccc6ccccc65)c4)c4ccc5c(c4)sc4ccccc45)c4ccccc34)c3c2oc2ccccc23)cc1 ; c1ccc(-c2ccc(-c3ccc(N(c4ccc5c(ccc6ccccc65)c4)c4cccc5c4sc4ccccc45)c4ccccc34)c3c2oc2ccccc23)cc1 ; c1ccc(-c2ccc(-c3ccc(N(c4ccc(-c5cccc6c5sc5ccccc56)cc4)c4ccc5c(ccc6ccccc65)c4)c4ccccc34)c3c2oc2ccccc23)cc1]

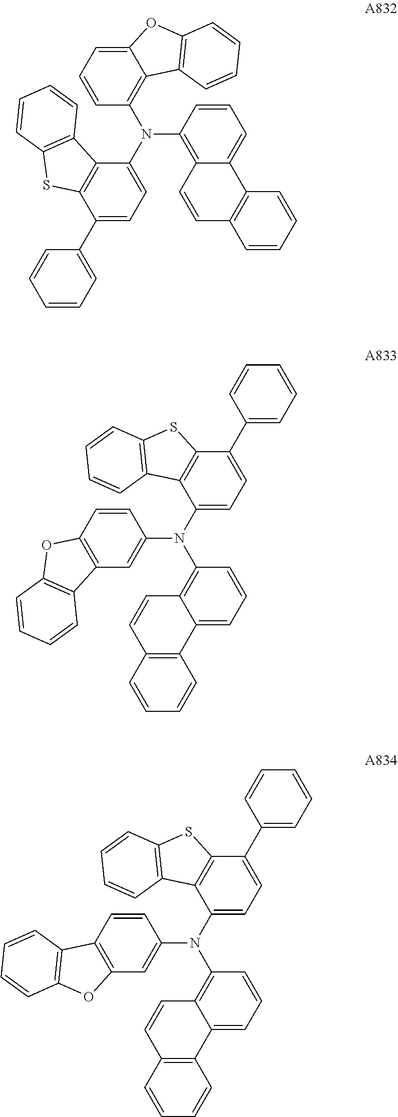 [SMILES: c1ccc(-c2ccc(N(c3cccc4c3ccc3ccccc34)c3cccc4oc5ccccc5c34)c3c2sc2ccccc23)cc1 ; c1ccc(-c2ccc(N(c3ccc4oc5ccccc5c4c3)c3cccc4c3ccc3ccccc34)c3c2sc2ccccc23)cc1 ; c1ccc(-c2ccc(N(c3ccc4c(c3)oc3ccccc34)c3cccc4c3ccc3ccccc34)c3c2sc2ccccc23)cc1]

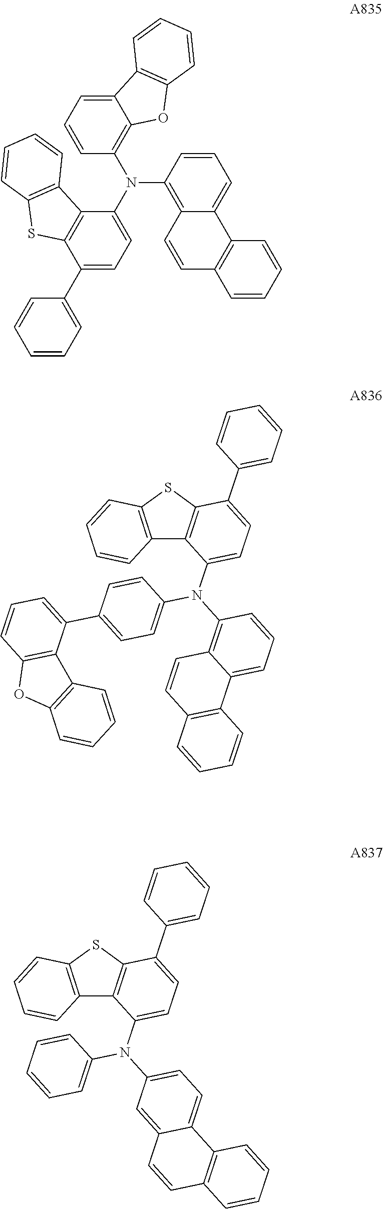 [SMILES: c1ccc(-c2ccc(N(c3cccc4c3ccc3ccccc34)c3cccc4c3oc3ccccc34)c3c2sc2ccccc23)cc1 ; c1ccc(-c2ccc(N(c3ccc(-c4cccc5oc6ccccc6c45)cc3)c3cccc4c3ccc3ccccc34)c3c2sc2ccccc23)cc1 ; c1ccc(-c2ccc(N(c3ccccc3)c3ccc4c(ccc5ccccc54)c3)c3c2sc2ccccc23)cc1]

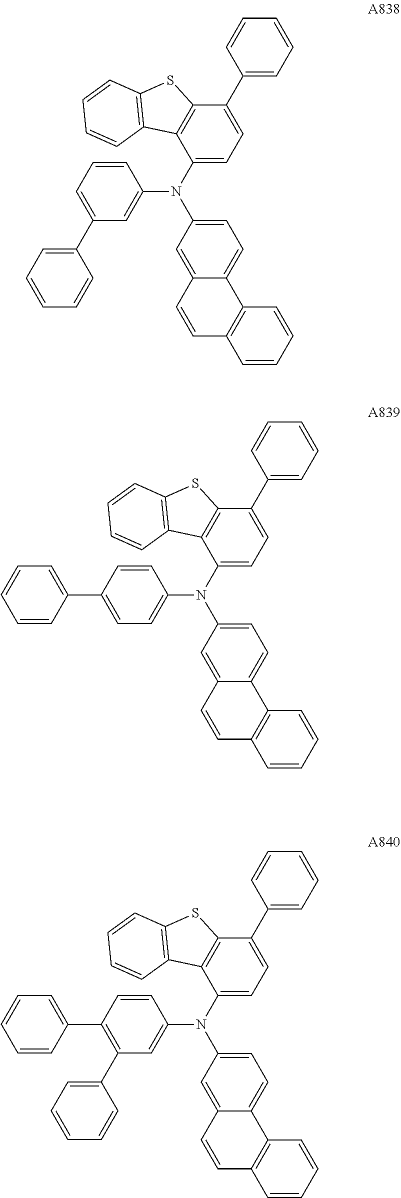 [SMILES: c1ccc(-c2cccc(N(c3ccc4c(ccc5ccccc54)c3)c3ccc(-c4ccccc4)c4sc5ccccc5c34)c2)cc1 ; c1ccc(-c2ccc(N(c3ccc4c(ccc5ccccc54)c3)c3ccc(-c4ccccc4)c4sc5ccccc5c34)cc2)cc1 ; c1ccc(-c2ccc(N(c3ccc4c(ccc5ccccc54)c3)c3ccc(-c4ccccc4)c4sc5ccccc5c34)cc2-c2ccccc2)cc1]

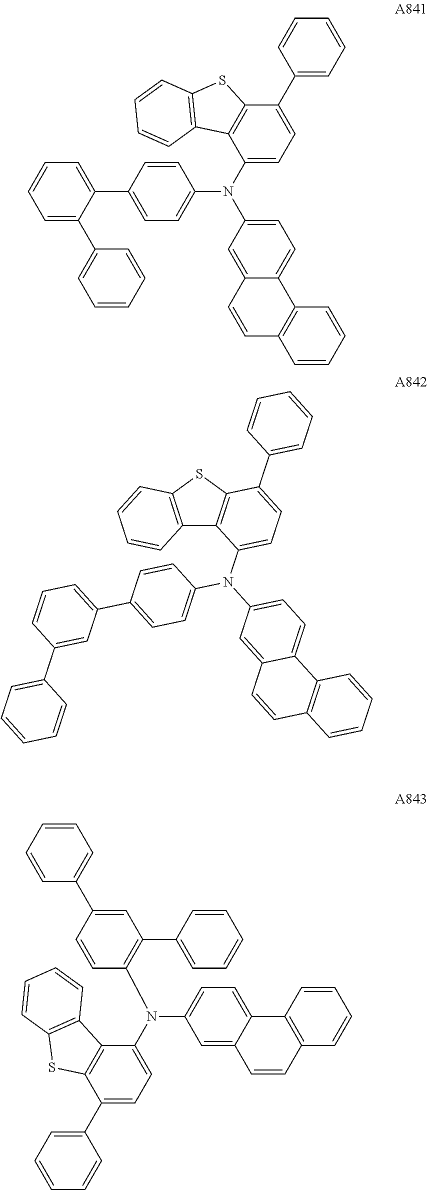

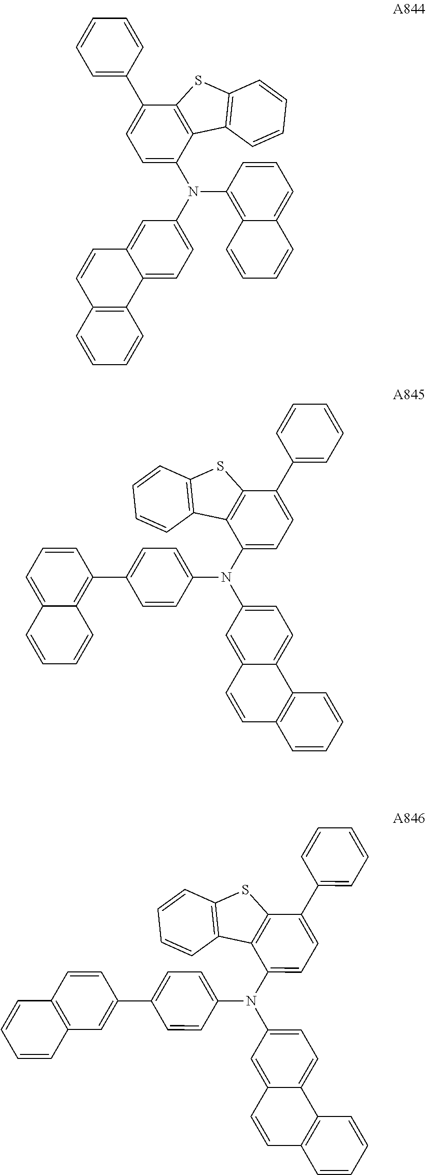 [SMILES: c1ccc(-c2ccc(N(c3ccc4c(ccc5ccccc54)c3)c3cccc4ccccc34)c3c2sc2ccccc23)cc1 ; c1ccc(-c2ccc(N(c3ccc(-c4cccc5ccccc45)cc3)c3ccc4c(ccc5ccccc54)c3)c3c2sc2ccccc23)cc1 ; c1ccc(-c2ccc(N(c3ccc(-c4ccc5ccccc5c4)cc3)c3ccc4c(ccc5ccccc54)c3)c3c2sc2ccccc23)cc1]

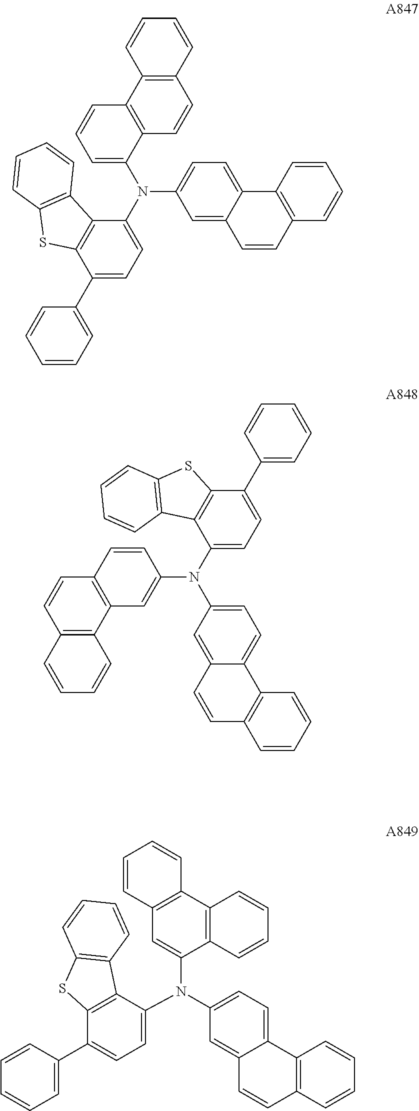 [SMILES: c1ccc(-c2ccc(N(c3ccc4c(ccc5ccccc54)c3)c3cccc4c3ccc3ccccc34)c3c2sc2ccccc23)cc1 ; c1ccc(-c2ccc(N(c3ccc4c(ccc5ccccc54)c3)c3ccc4ccc5ccccc5c4c3)c3c2sc2ccccc23)cc1 ; c1ccc(-c2ccc(N(c3ccc4c(ccc5ccccc54)c3)c3cc4ccccc4c4ccccc34)c3c2sc2ccccc23)cc1]

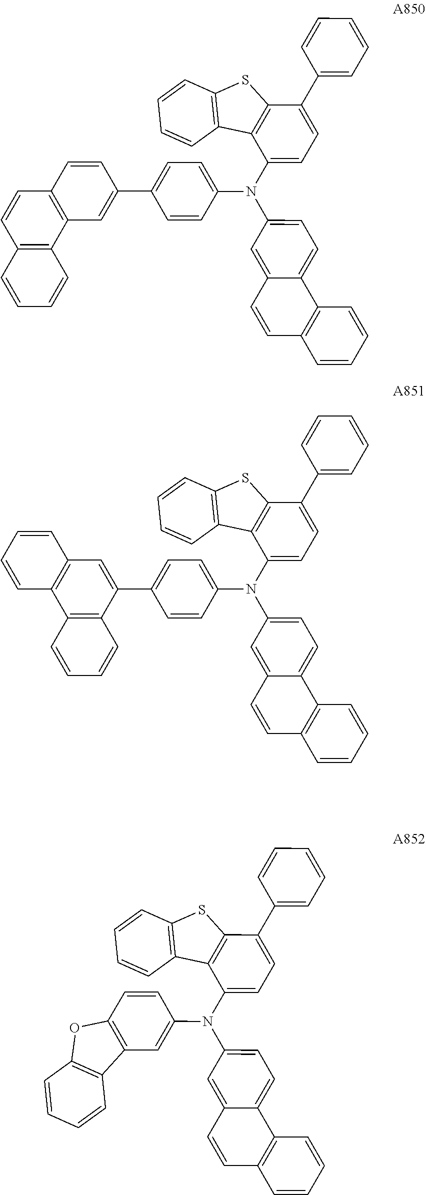 [SMILES: c1ccc(-c2ccc(N(c3ccc(-c4ccc5ccc6ccccc6c5c4)cc3)c3ccc4c(ccc5ccccc54)c3)c3c2sc2ccccc23)cc1 ; c1ccc(-c2ccc(N(c3ccc(-c4cc5ccccc5c5ccccc45)cc3)c3ccc4c(ccc5ccccc54)c3)c3c2sc2ccccc23)cc1 ; c1ccc(-c2ccc(N(c3ccc4c(ccc5ccccc54)c3)c3ccc4oc5ccccc5c4c3)c3c2sc2ccccc23)cc1]

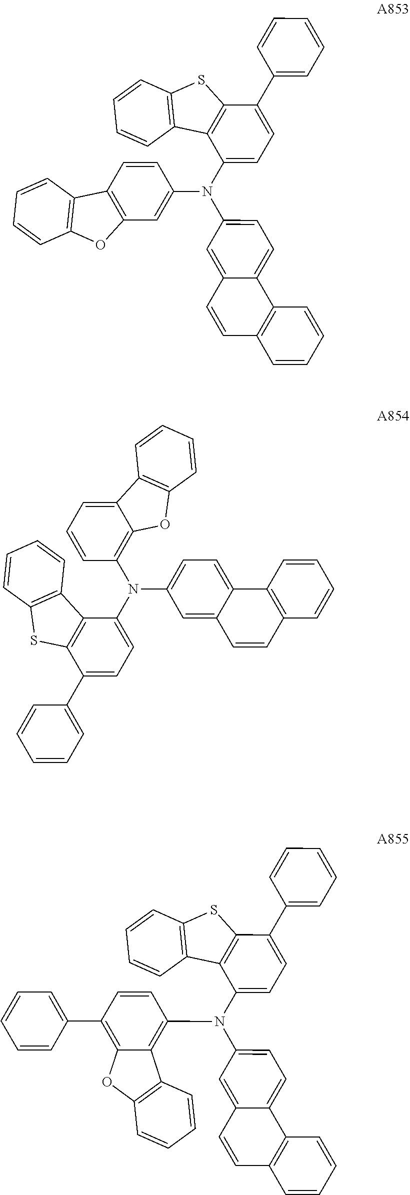 [SMILES: c1ccc(-c2ccc(N(c3ccc4c(ccc5ccccc54)c3)c3ccc4c(c3)oc3ccccc34)c3c2sc2ccccc23)cc1 ; c1ccc(-c2ccc(N(c3ccc4c(ccc5ccccc54)c3)c3cccc4c3oc3ccccc34)c3c2sc2ccccc23)cc1 ; c1ccc(-c2ccc(N(c3ccc4c(ccc5ccccc54)c3)c3ccc(-c4ccccc4)c4sc5ccccc5c34)c3c2oc2ccccc23)cc1]

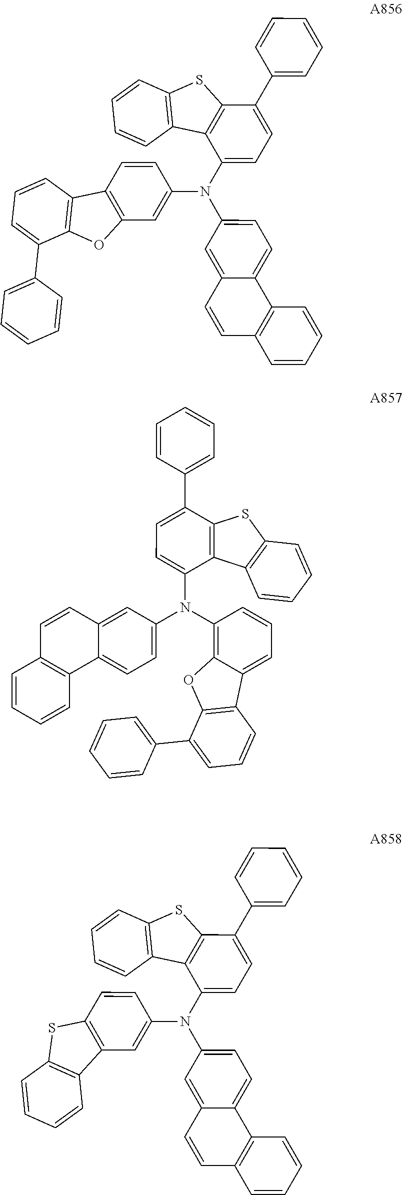 [SMILES: c1ccc(-c2cccc3c2oc2cc(N(c4ccc5c(ccc6ccccc65)c4)c4ccc(-c5ccccc5)c5sc6ccccc6c45)ccc23)cc1 ; c1ccc(-c2cccc3c2oc2c(N(c4ccc5c(ccc6ccccc65)c4)c4ccc(-c5ccccc5)c5sc6ccccc6c45)cccc23)cc1 ; c1ccc(-c2ccc(N(c3ccc4c(ccc5ccccc54)c3)c3ccc4sc5ccccc5c4c3)c3c2sc2ccccc23)cc1]

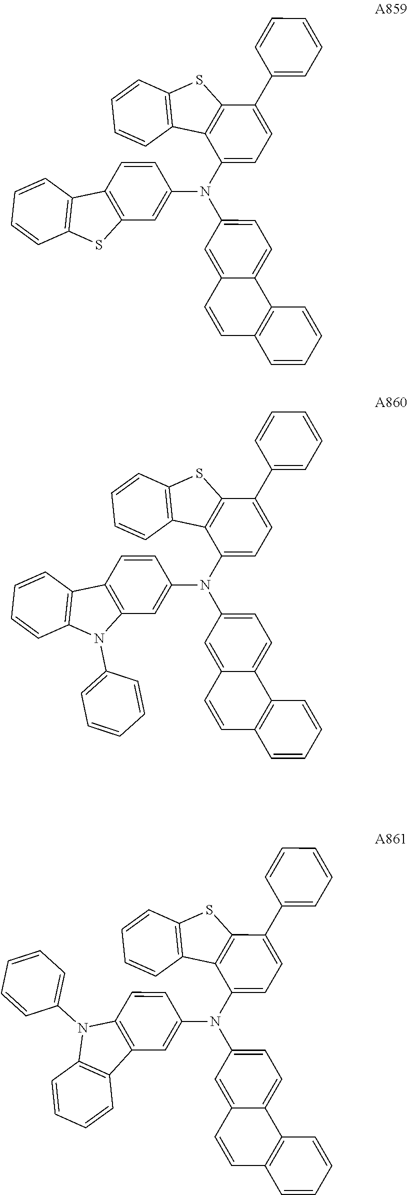 [SMILES: c1ccc(-c2ccc(N(c3ccc4c(ccc5ccccc54)c3)c3ccc4c(c3)sc3ccccc34)c3c2sc2ccccc23)cc1 ; c1ccc(-c2ccc(N(c3ccc4c(ccc5ccccc54)c3)c3ccc4c5ccccc5n(-c5ccccc5)c4c3)c3c2sc2ccccc23)cc1 ; c1ccc(-c2ccc(N(c3ccc4c(ccc5ccccc54)c3)c3ccc4c(c3)c3ccccc3n4-c3ccccc3)c3c2sc2ccccc23)cc1]

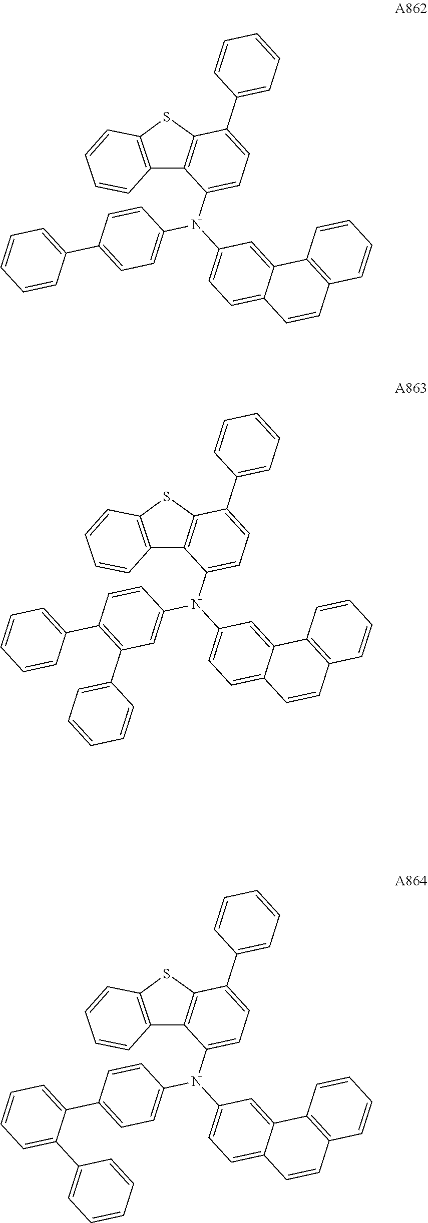 [SMILES: c1ccc(-c2ccc(N(c3ccc4ccc5ccccc5c4c3)c3ccc(-c4ccccc4)c4sc5ccccc5c34)cc2)cc1 ; c1ccc(-c2ccc(N(c3ccc4ccc5ccccc5c4c3)c3ccc(-c4ccccc4)c4sc5ccccc5c34)cc2-c2ccccc2)cc1 ; c1ccc(-c2ccccc2-c2ccc(N(c3ccc4ccc5ccccc5c4c3)c3ccc(-c4ccccc4)c4sc5ccccc5c34)cc2)cc1]

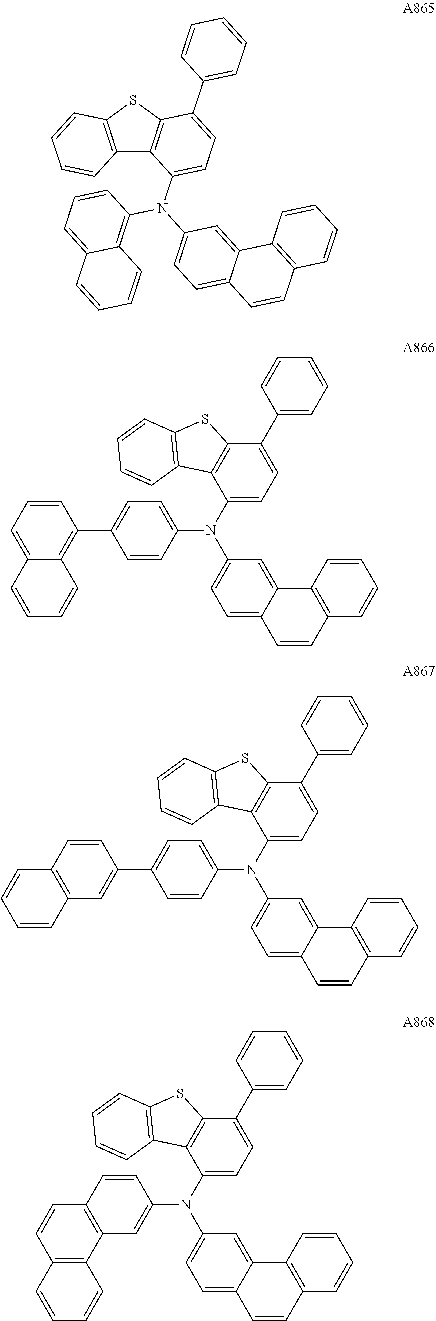 [SMILES: c1ccc(-c2ccc(N(c3ccc4ccc5ccccc5c4c3)c3cccc4ccccc34)c3c2sc2ccccc23)cc1 ; c1ccc(-c2ccc(N(c3ccc(-c4cccc5ccccc45)cc3)c3ccc4ccc5ccccc5c4c3)c3c2sc2ccccc23)cc1 ; c1ccc(-c2ccc(N(c3ccc(-c4ccc5ccccc5c4)cc3)c3ccc4ccc5ccccc5c4c3)c3c2sc2ccccc23)cc1 ; c1ccc(-c2ccc(N(c3ccc4ccc5ccccc5c4c3)c3ccc4ccc5ccccc5c4c3)c3c2sc2ccccc23)cc1]

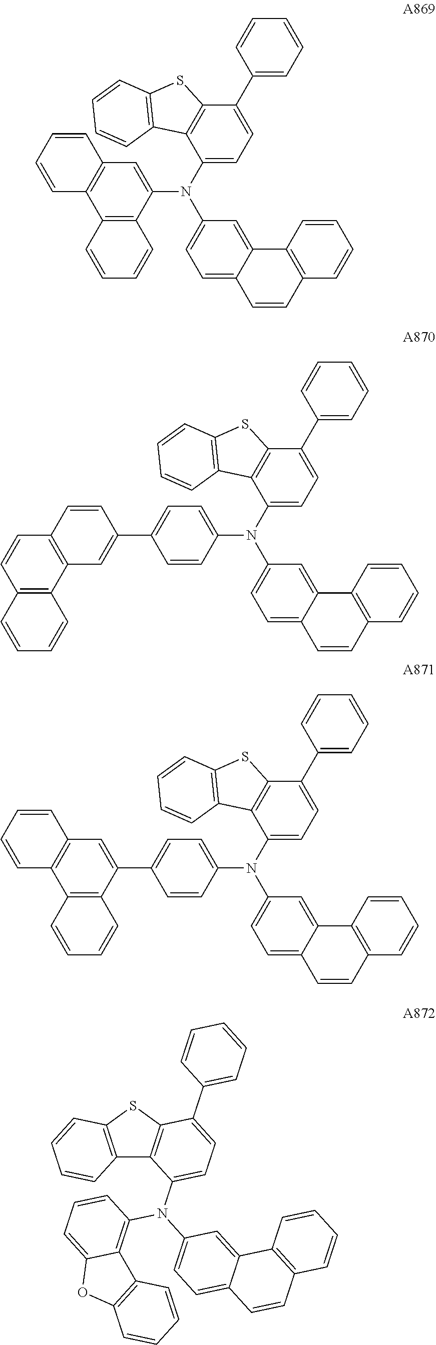

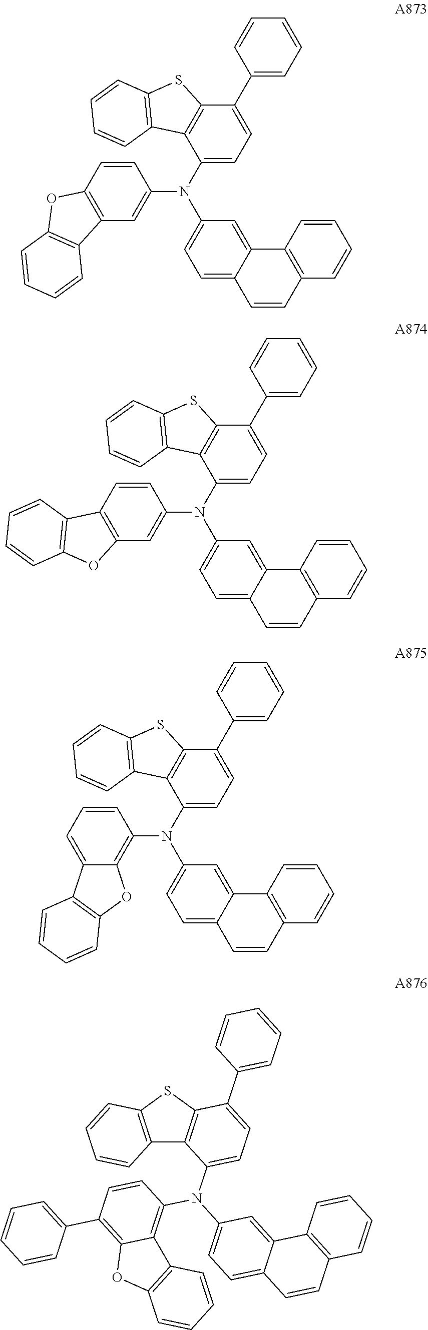

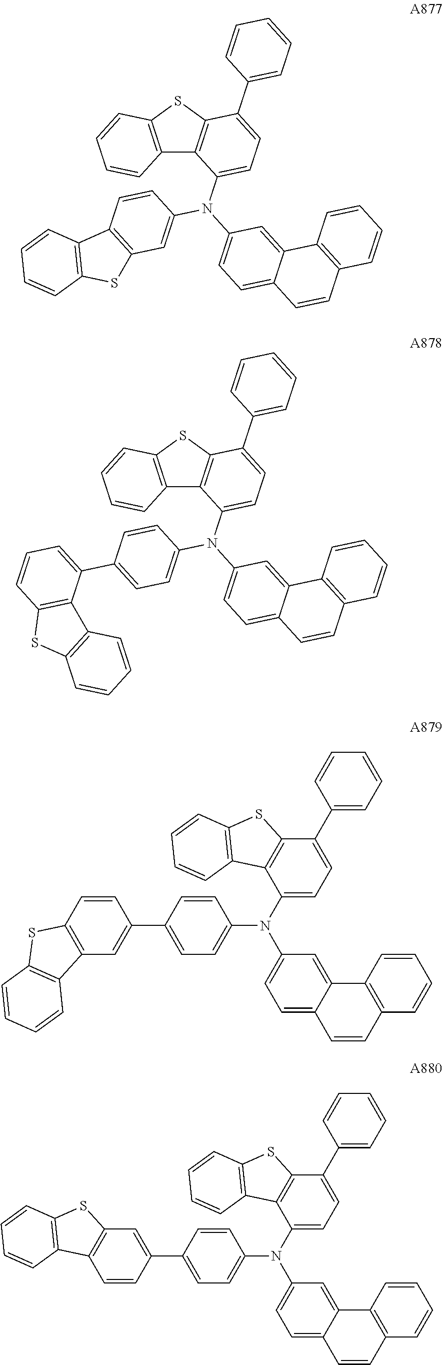 [SMILES: c1ccc(-c2ccc(N(c3ccc4c(c3)sc3ccccc34)c3ccc4ccc5ccccc5c4c3)c3c2sc2ccccc23)cc1 ; c1ccc(-c2ccc(N(c3ccc(-c4cccc5sc6ccccc6c45)cc3)c3ccc4ccc5ccccc5c4c3)c3c2sc2ccccc23)cc1 ; c1ccc(-c2ccc(N(c3ccc(-c4ccc5sc6ccccc6c5c4)cc3)c3ccc4ccc5ccccc5c4c3)c3c2sc2ccccc23)cc1 ; c1ccc(-c2ccc(N(c3ccc(-c4ccc5c(c4)sc4ccccc45)cc3)c3ccc4ccc5ccccc5c4c3)c3c2sc2ccccc23)cc1]

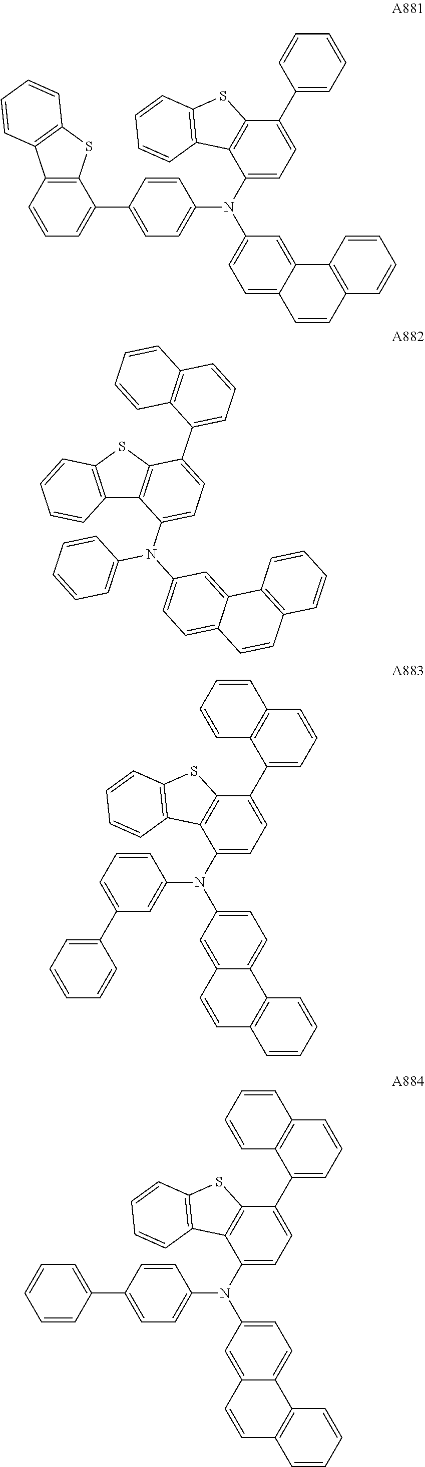

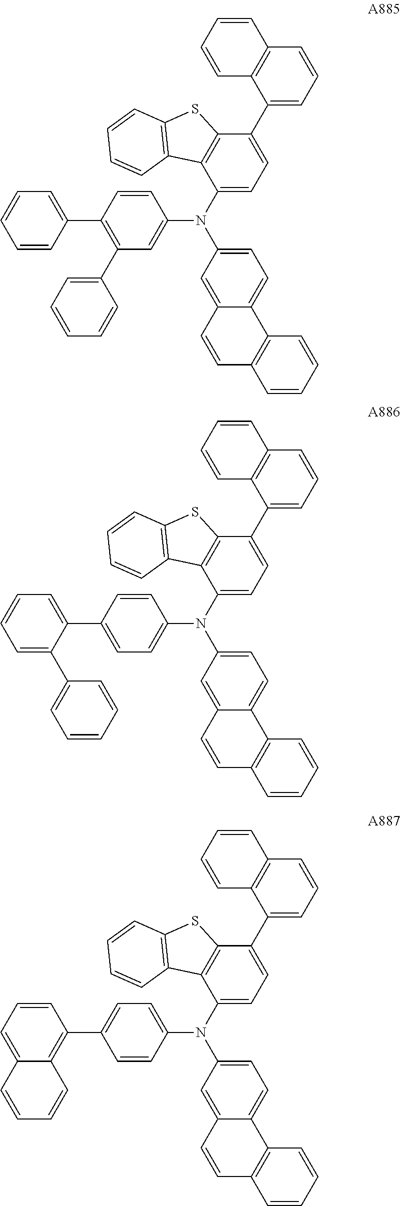

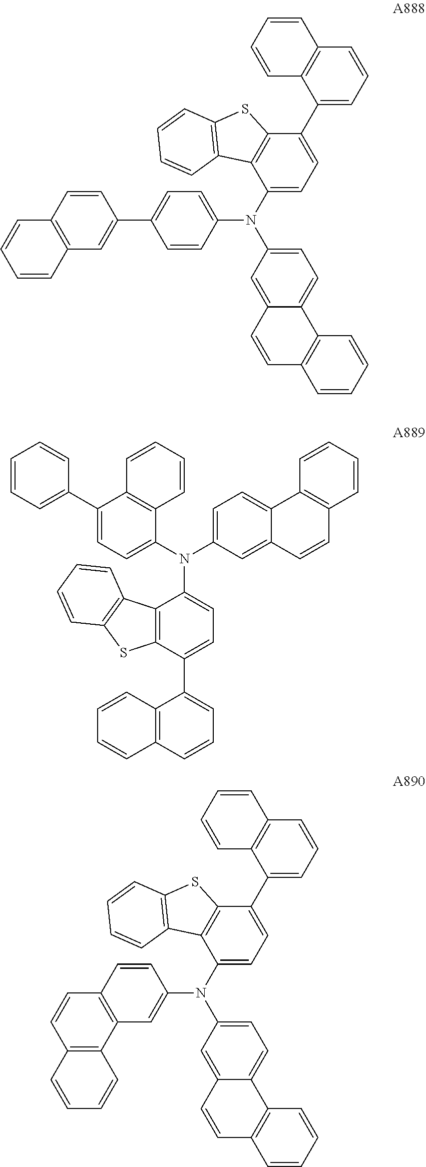

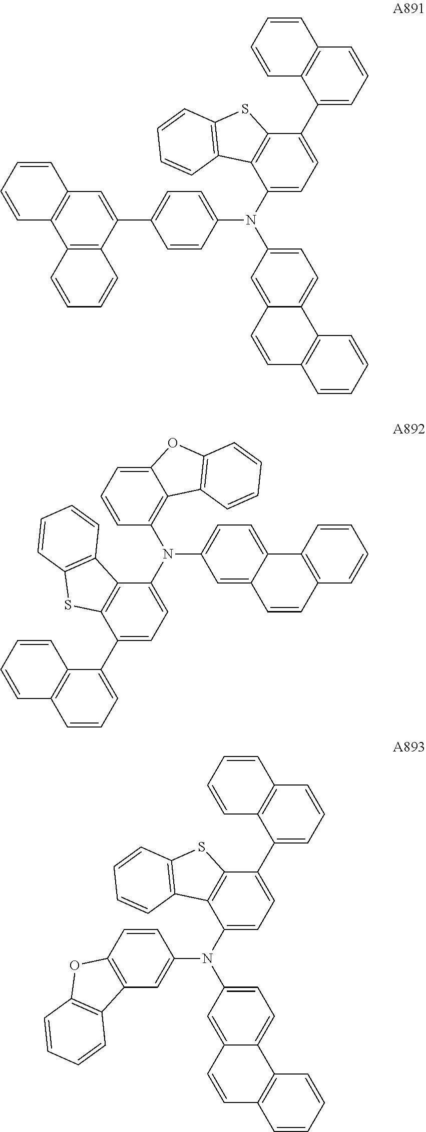 [SMILES: c1ccc2c(-c3ccc(N(c4ccc(-c5cc6ccccc6c6ccccc56)cc4)c4ccc5c(ccc6ccccc65)c4)c4c3sc3ccccc34)cccc2c1 ; c1ccc2c(-c3ccc(N(c4ccc5c(ccc6ccccc65)c4)c4cccc5oc6ccccc6c45)c4c3sc3ccccc34)cccc2c1 ; c1ccc2c(-c3ccc(N(c4ccc5c(ccc6ccccc65)c4)c4ccc5oc6ccccc6c5c4)c4c3sc3ccccc34)cccc2c1]

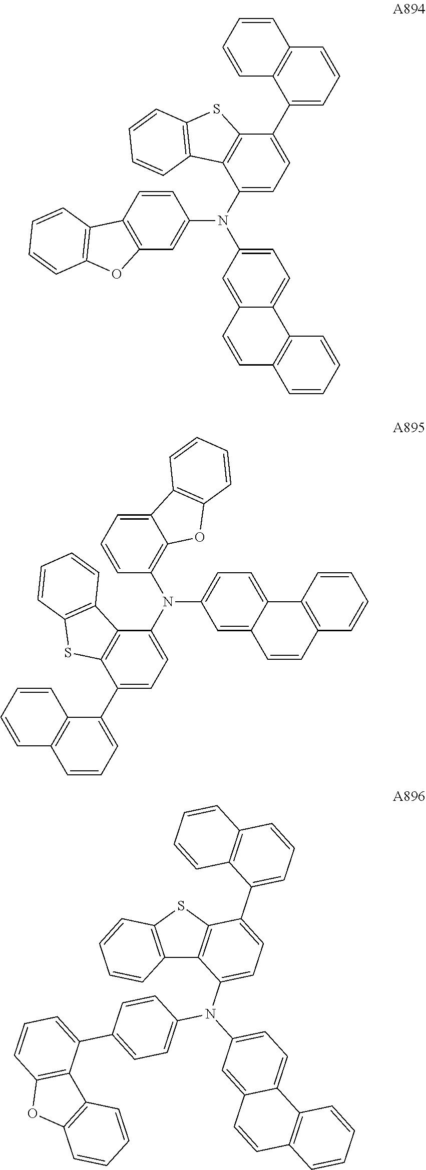 [SMILES: c1ccc2c(-c3ccc(N(c4ccc5c(ccc6ccccc65)c4)c4ccc5c(c4)oc4ccccc45)c4c3sc3ccccc34)cccc2c1 ; c1ccc2c(-c3ccc(N(c4ccc5c(ccc6ccccc65)c4)c4cccc5c4oc4ccccc45)c4c3sc3ccccc34)cccc2c1 ; c1ccc2c(-c3ccc(N(c4ccc(-c5cccc6oc7ccccc7c56)cc4)c4ccc5c(ccc6ccccc65)c4)c4c3sc3ccccc34)cccc2c1]

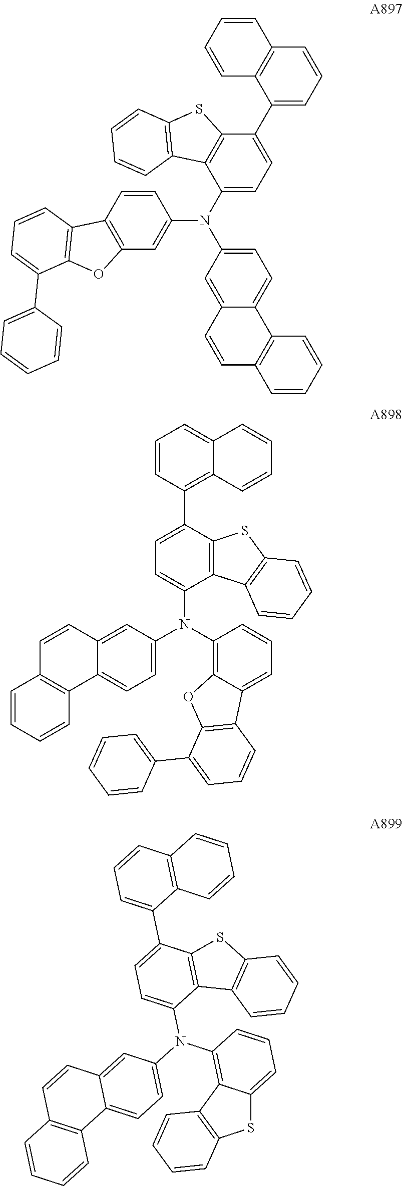

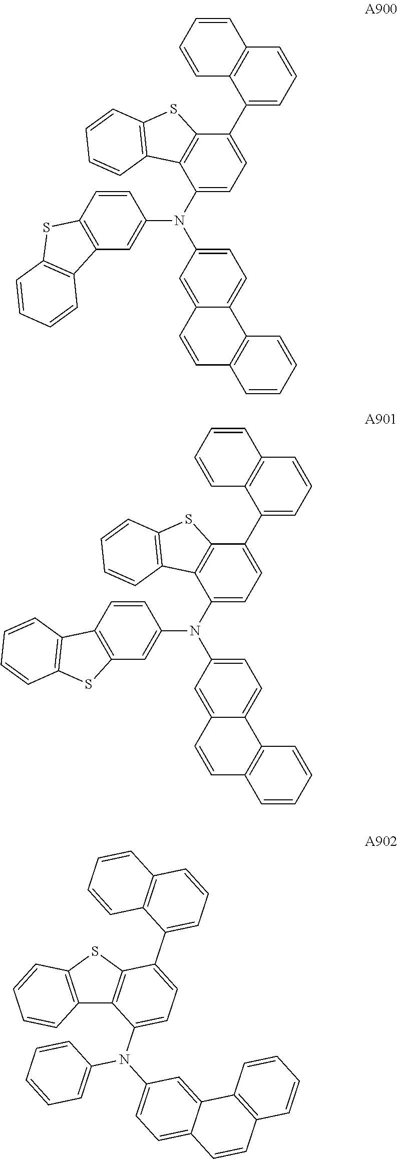

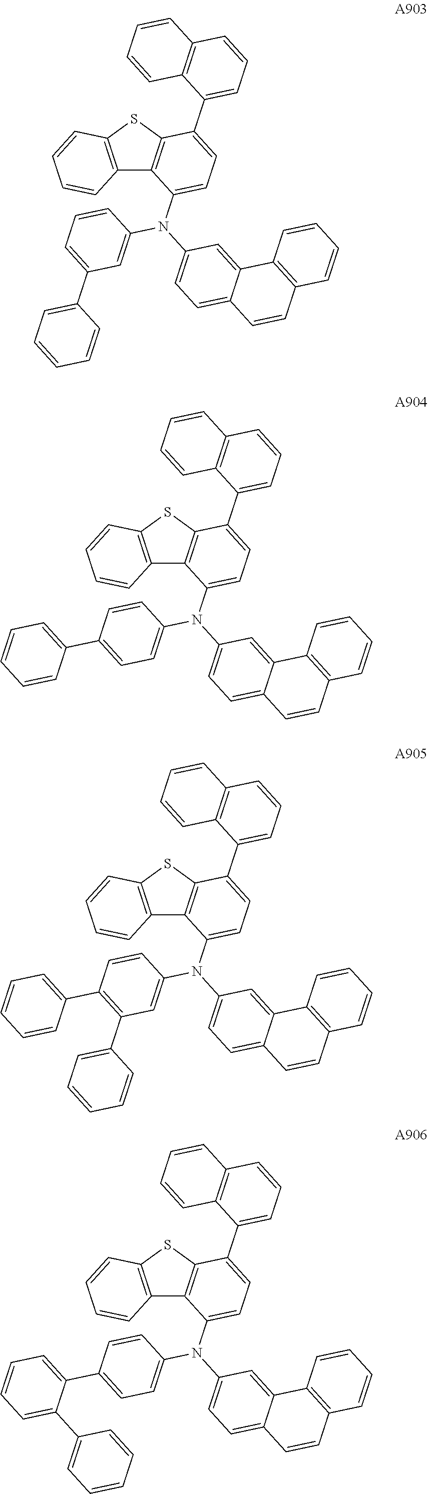 [SMILES: c1ccc(-c2cccc(N(c3ccc4ccc5ccccc5c4c3)c3ccc(-c4cccc5ccccc45)c4sc5ccccc5c34)c2)cc1 ; c1ccc(-c2ccc(N(c3ccc4ccc5ccccc5c4c3)c3ccc(-c4cccc5ccccc45)c4sc5ccccc5c34)cc2)cc1 ; c1ccc(-c2ccc(N(c3ccc4ccc5ccccc5c4c3)c3ccc(-c4cccc5ccccc45)c4sc5ccccc5c34)cc2-c2ccccc2)cc1 ; c1ccc(-c2ccccc2-c2ccc(N(c3ccc4ccc5ccccc5c4c3)c3ccc(-c4cccc5ccccc45)c4sc5ccccc5c34)cc2)cc1]

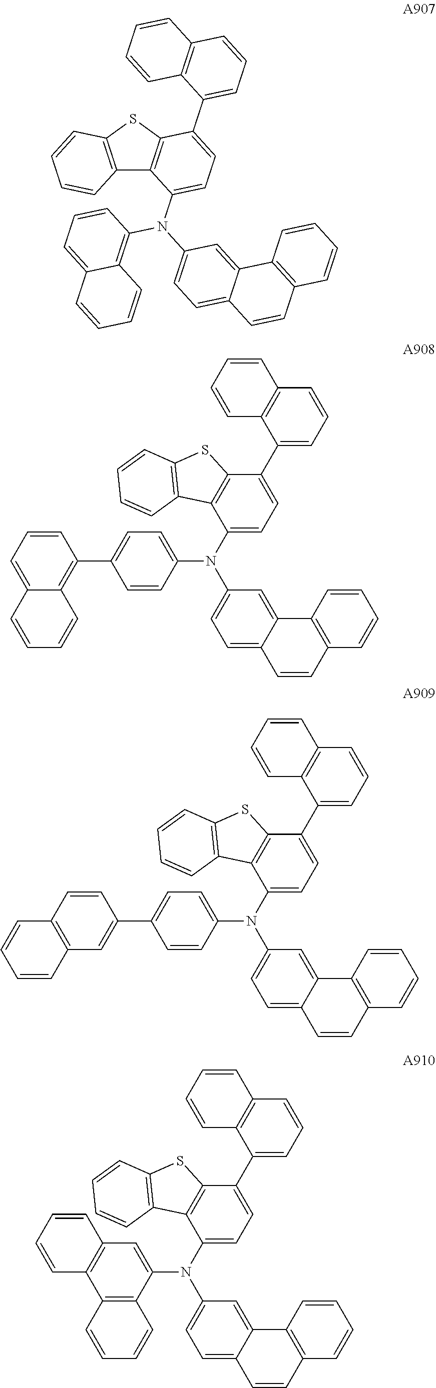

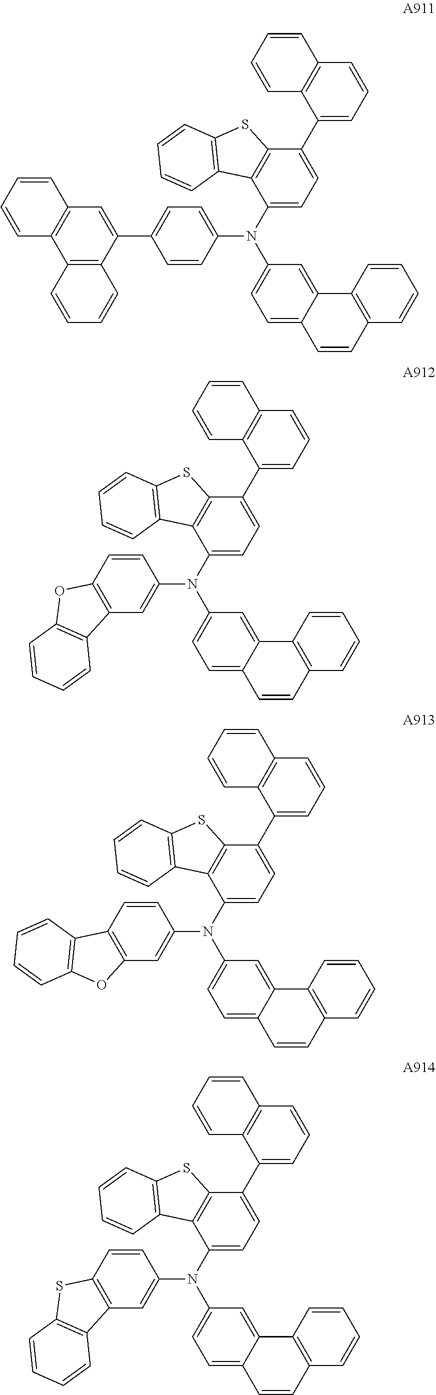

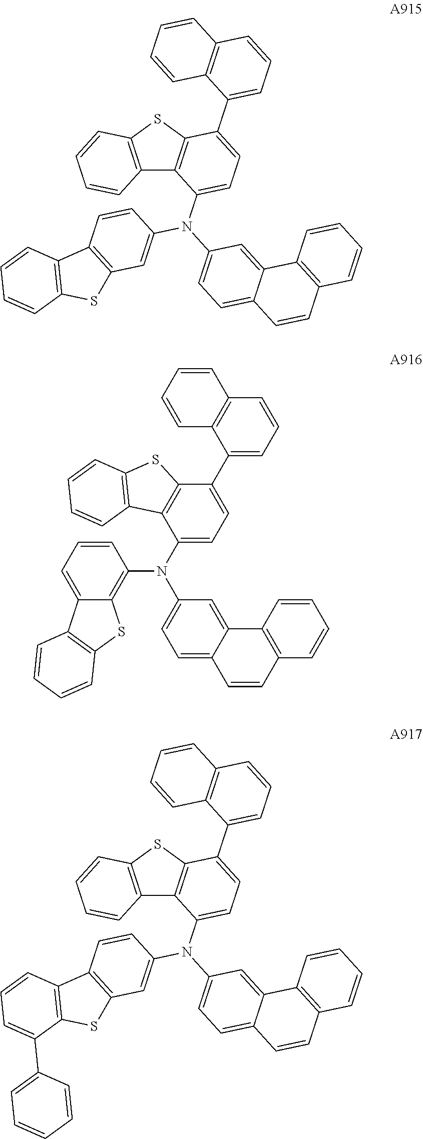 [SMILES: c1ccc2c(-c3ccc(N(c4ccc5c(c4)sc4ccccc45)c4ccc5ccc6ccccc6c5c4)c4c3sc3ccccc34)cccc2c1 ; c1ccc2c(-c3ccc(N(c4ccc5ccc6ccccc6c5c4)c4cccc5c4sc4ccccc45)c4c3sc3ccccc34)cccc2c1 ; c1ccc(-c2cccc3c2sc2cc(N(c4ccc5ccc6ccccc6c5c4)c4ccc(-c5cccc6ccccc56)c5sc6ccccc6c45)ccc23)cc1]

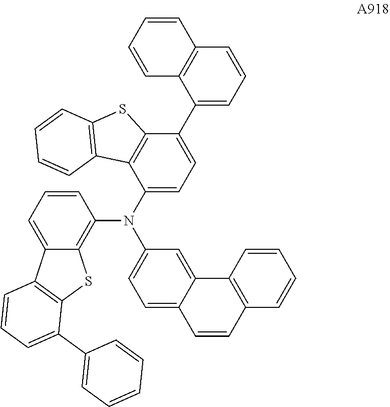 [SMILES: c1ccc(-c2cccc3c2sc2c(N(c4ccc5ccc6ccccc6c5c4)c4ccc(-c5cccc6ccccc56)c5sc6ccccc6c45)cccc23)cc1]

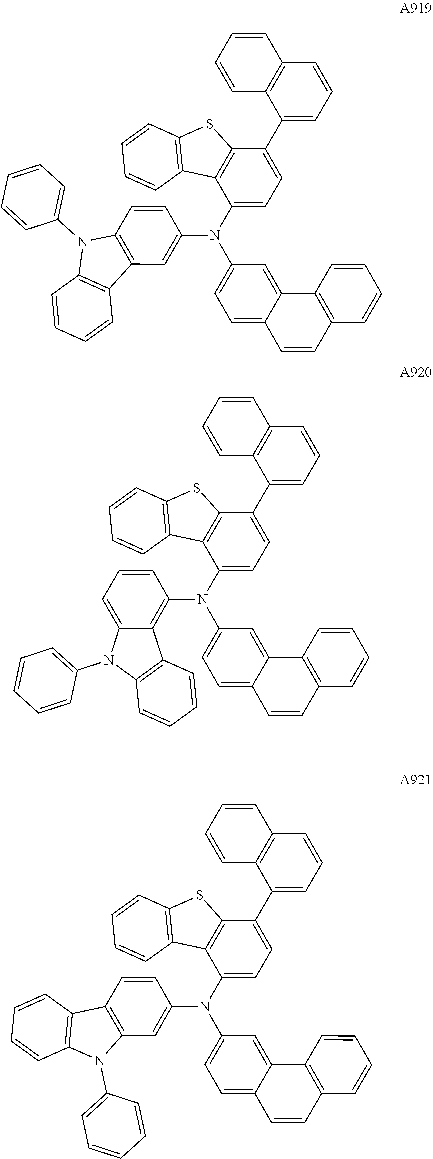 [SMILES: c1ccc(-n2c3ccccc3c3cc(N(c4ccc5ccc6ccccc6c5c4)c4ccc(-c5cccc6ccccc56)c5sc6ccccc6c45)ccc32)cc1 ; c1ccc(-n2c3ccccc3c3c(N(c4ccc5ccc6ccccc6c5c4)c4ccc(-c5cccc6ccccc56)c5sc6ccccc6c45)cccc32)cc1 ; c1ccc(-n2c3ccccc3c3ccc(N(c4ccc5ccc6ccccc6c5c4)c4ccc(-c5cccc6ccccc56)c5sc6ccccc6c45)cc32)cc1]

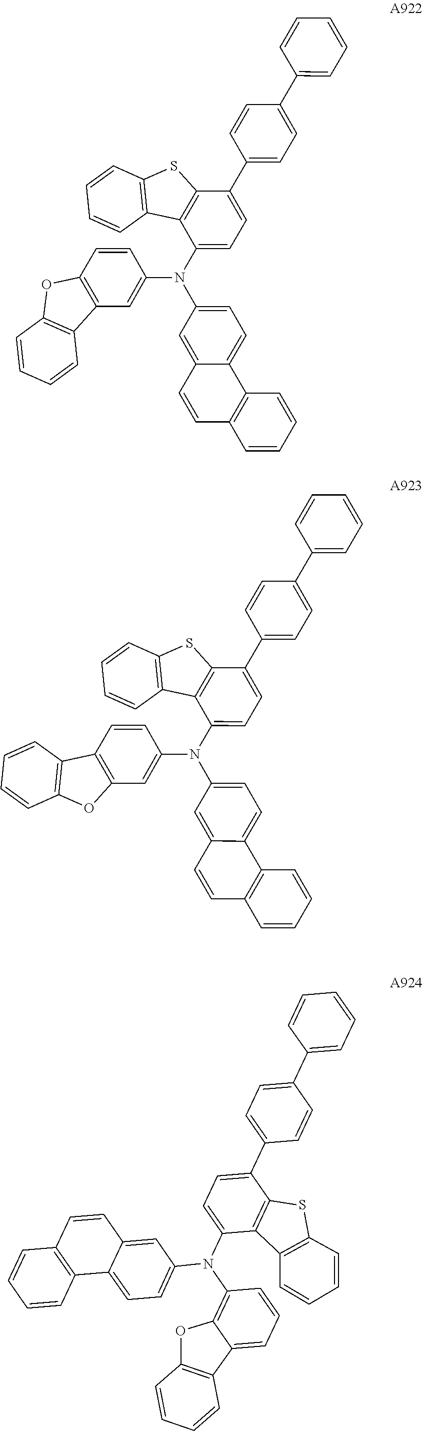 [SMILES: c1ccc(-c2ccc(-c3ccc(N(c4ccc5c(ccc6ccccc65)c4)c4ccc5oc6ccccc6c5c4)c4c3sc3ccccc34)cc2)cc1 ; c1ccc(-c2ccc(-c3ccc(N(c4ccc5c(ccc6ccccc65)c4)c4ccc5c(c4)oc4ccccc45)c4c3sc3ccccc34)cc2)cc1 ; c1ccc(-c2ccc(-c3ccc(N(c4ccc5c(ccc6ccccc65)c4)c4cccc5c4oc4ccccc45)c4c3sc3ccccc34)cc2)cc1]

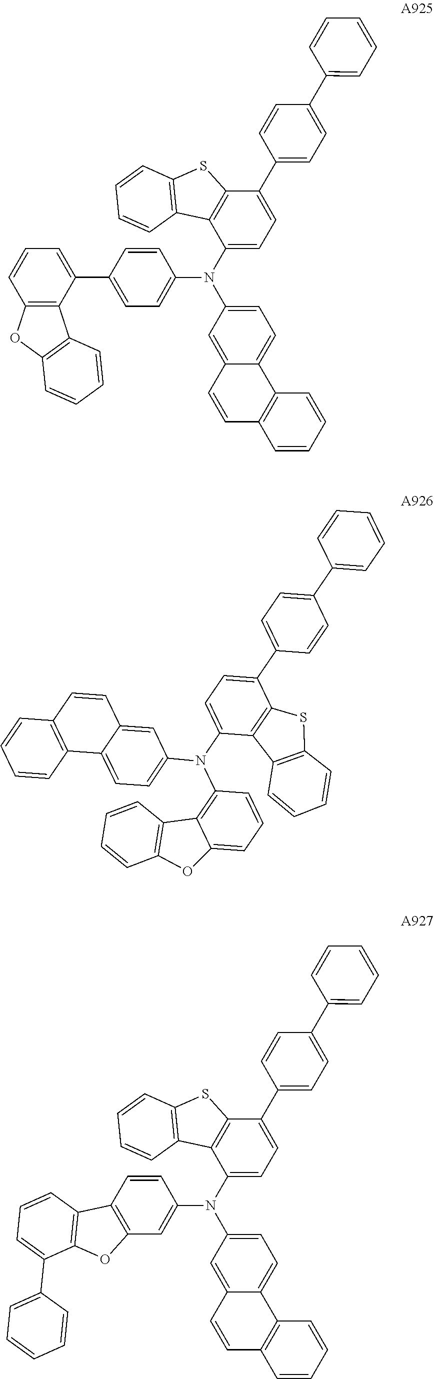 [SMILES: c1ccc(-c2ccc(-c3ccc(N(c4ccc(-c5cccc6oc7ccccc7c56)cc4)c4ccc5c(ccc6ccccc65)c4)c4c3sc3ccccc34)cc2)cc1 ; c1ccc(-c2ccc(-c3ccc(N(c4ccc5c(ccc6ccccc65)c4)c4cccc5oc6ccccc6c45)c4c3sc3ccccc34)cc2)cc1 ; c1ccc(-c2ccc(-c3ccc(N(c4ccc5c(ccc6ccccc65)c4)c4ccc5c(c4)oc4c(-c6ccccc6)cccc45)c4c3sc3ccccc34)cc2)cc1]

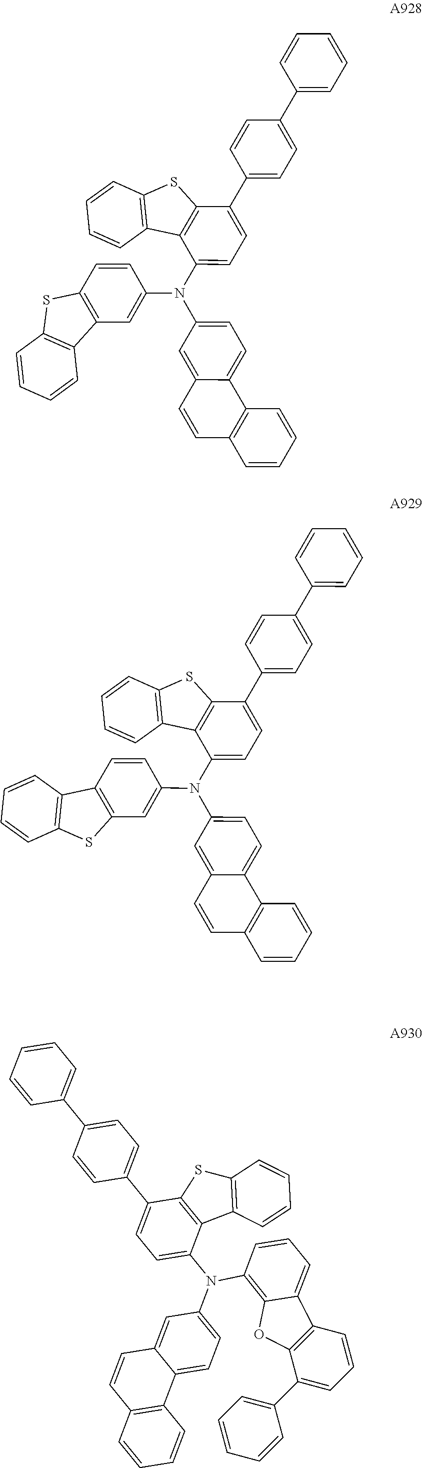 [SMILES: c1ccc(-c2ccc(-c3ccc(N(c4ccc5c(ccc6ccccc65)c4)c4ccc5sc6ccccc6c5c4)c4c3sc3ccccc34)cc2)cc1 ; c1ccc(-c2ccc(-c3ccc(N(c4ccc5c(ccc6ccccc65)c4)c4ccc5c(c4)sc4ccccc45)c4c3sc3ccccc34)cc2)cc1 ; c1ccc(-c2ccc(-c3ccc(N(c4ccc5c(ccc6ccccc65)c4)c4cccc5c4oc4c(-c6ccccc6)cccc45)c4c3sc3ccccc34)cc2)cc1]

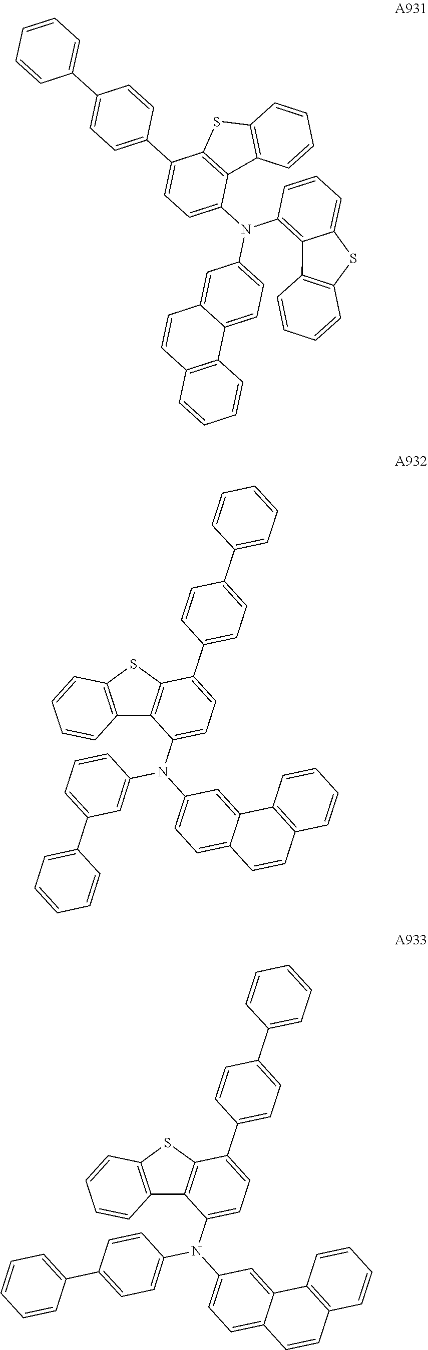 [SMILES: c1ccc(-c2ccc(-c3ccc(N(c4ccc5c(ccc6ccccc65)c4)c4cccc5sc6ccccc6c45)c4c3sc3ccccc34)cc2)cc1 ; c1ccc(-c2ccc(-c3ccc(N(c4cccc(-c5ccccc5)c4)c4ccc5ccc6ccccc6c5c4)c4c3sc3ccccc34)cc2)cc1 ; c1ccc(-c2ccc(-c3ccc(N(c4ccc(-c5ccccc5)cc4)c4ccc5ccc6ccccc6c5c4)c4c3sc3ccccc34)cc2)cc1]

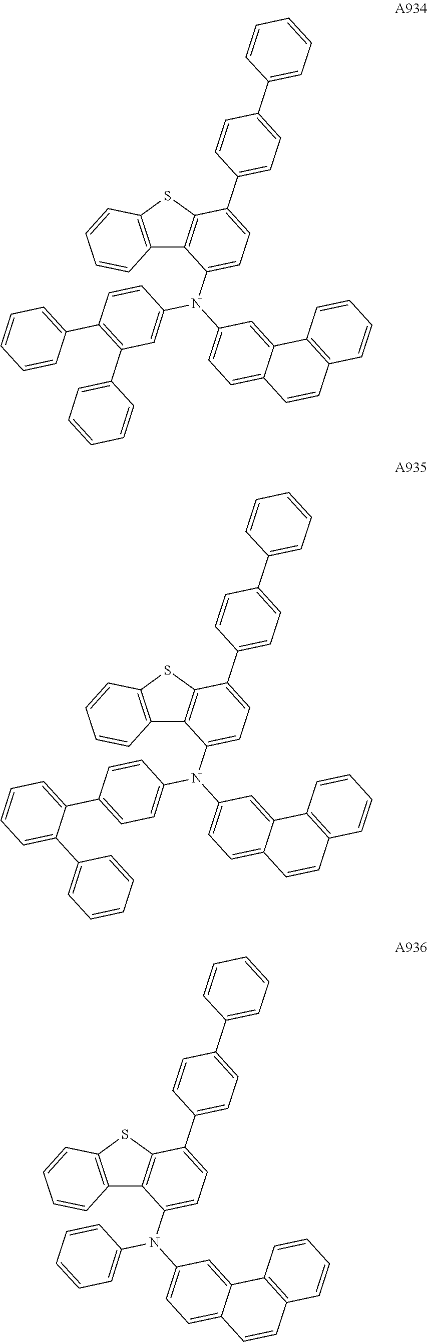 [SMILES: c1ccc(-c2ccc(-c3ccc(N(c4ccc(-c5ccccc5)c(-c5ccccc5)c4)c4ccc5ccc6ccccc6c5c4)c4c3sc3ccccc34)cc2)cc1 ; c1ccc(-c2ccc(-c3ccc(N(c4ccc(-c5ccccc5-c5ccccc5)cc4)c4ccc5ccc6ccccc6c5c4)c4c3sc3ccccc34)cc2)cc1 ; c1ccc(-c2ccc(-c3ccc(N(c4ccccc4)c4ccc5ccc6ccccc6c5c4)c4c3sc3ccccc34)cc2)cc1]

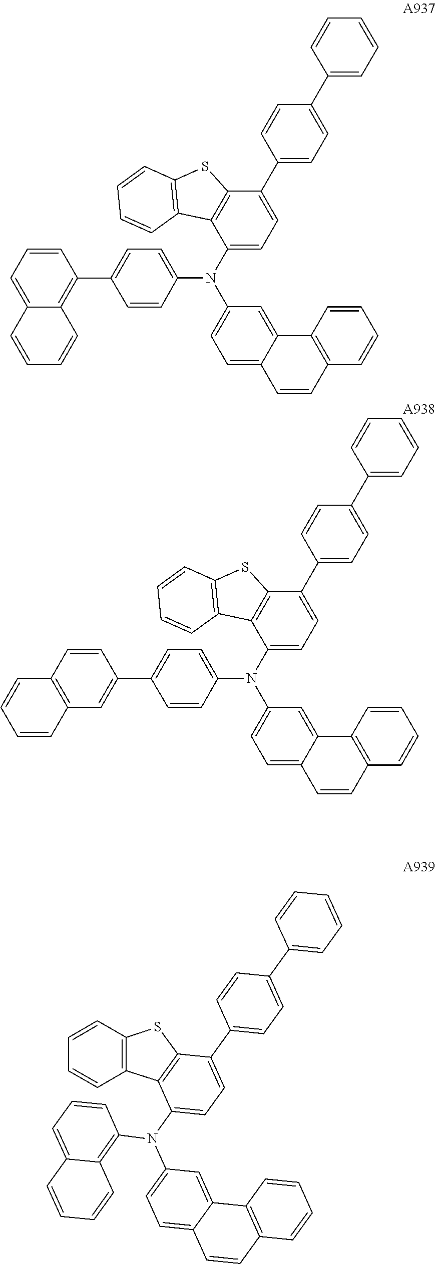 [SMILES: c1ccc(-c2ccc(-c3ccc(N(c4ccc(-c5cccc6ccccc56)cc4)c4ccc5ccc6ccccc6c5c4)c4c3sc3ccccc34)cc2)cc1 ; c1ccc(-c2ccc(-c3ccc(N(c4ccc(-c5ccc6ccccc6c5)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5ccccc45)c4c3sc3ccccc34)cc2)cc1]

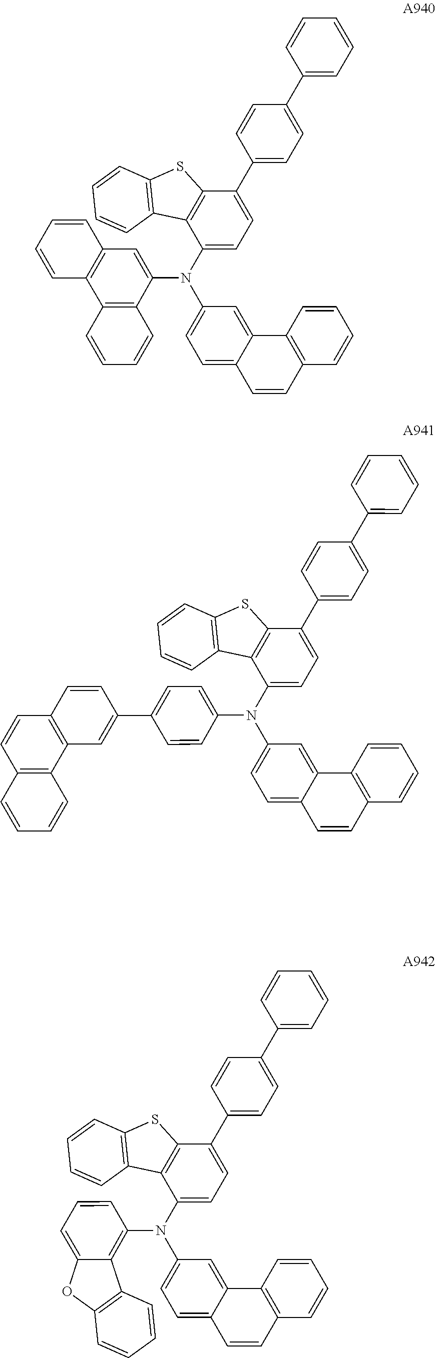 [SMILES: c1ccc(-c2ccc(-c3ccc(N(c4ccc5ccc6ccccc6c5c4)c4cc5ccccc5c5ccccc45)c4c3sc3ccccc34)cc2)cc1 ; c1ccc(-c2ccc(-c3ccc(N(c4ccc(-c5ccc6ccc7ccccc7c6c5)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5oc6ccccc6c45)c4c3sc3ccccc34)cc2)cc1]

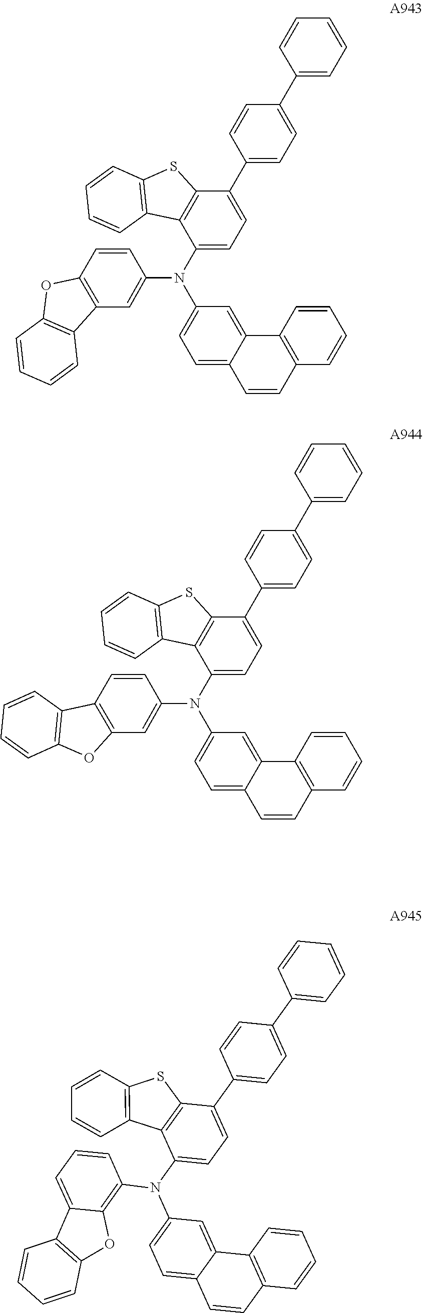 [SMILES: c1ccc(-c2ccc(-c3ccc(N(c4ccc5ccc6ccccc6c5c4)c4ccc5oc6ccccc6c5c4)c4c3sc3ccccc34)cc2)cc1 ; c1ccc(-c2ccc(-c3ccc(N(c4ccc5c(c4)oc4ccccc45)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5c4oc4ccccc45)c4c3sc3ccccc34)cc2)cc1]

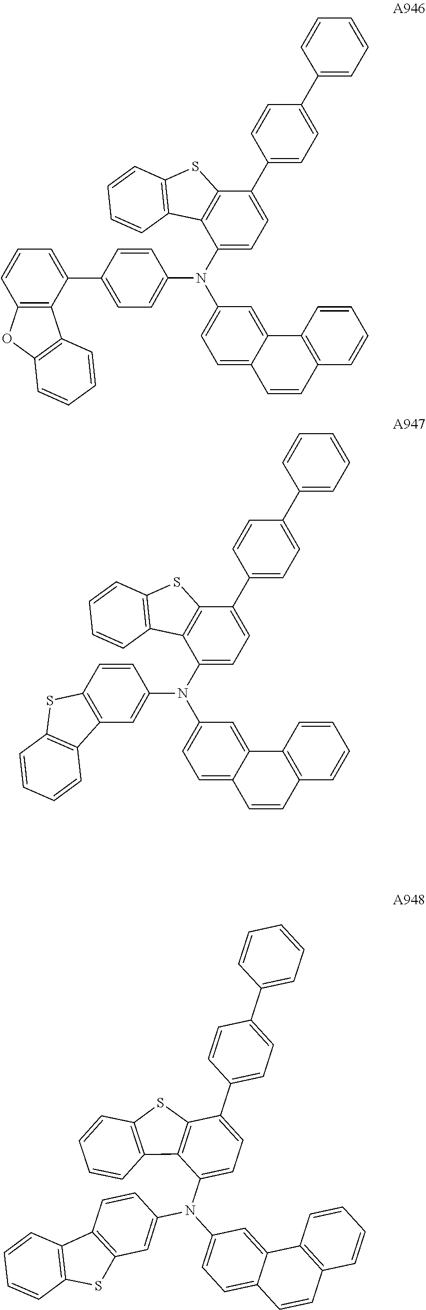

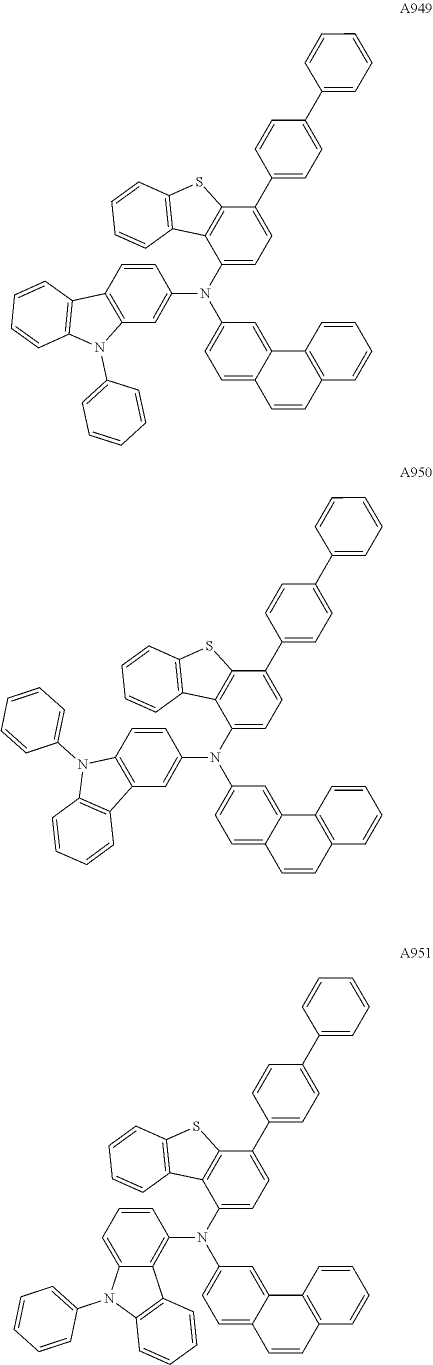 [SMILES: c1ccc(-c2ccc(-c3ccc(N(c4ccc5ccc6ccccc6c5c4)c4ccc5c6ccccc6n(-c6ccccc6)c5c4)c4c3sc3ccccc34)cc2)cc1 ; c1ccc(-c2ccc(-c3ccc(N(c4ccc5ccc6ccccc6c5c4)c4ccc5c(c4)c4ccccc4n5-c4ccccc4)c4c3sc3ccccc34)cc2)cc1 ; c1ccc(-c2ccc(-c3ccc(N(c4ccc5ccc6ccccc6c5c4)c4cccc5c4c4ccccc4n5-c4ccccc4)c4c3sc3ccccc34)cc2)cc1]

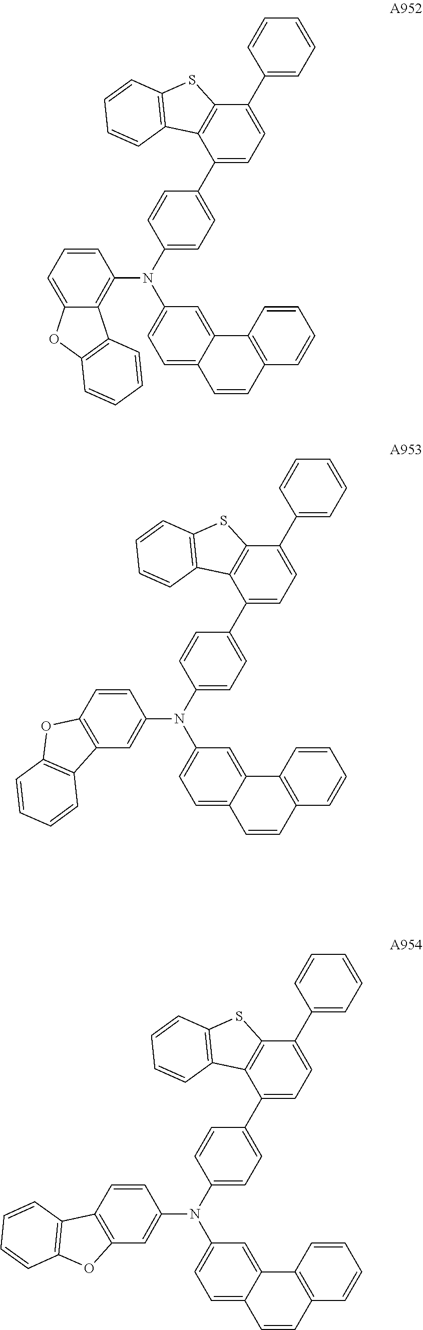 [SMILES: c1ccc(-c2ccc(-c3ccc(N(c4ccc5ccc6ccccc6c5c4)c4cccc5oc6ccccc6c45)cc3)c3c2sc2ccccc23)cc1 ; c1ccc(-c2ccc(-c3ccc(N(c4ccc5ccc6ccccc6c5c4)c4ccc5oc6ccccc6c5c4)cc3)c3c2sc2ccccc23)cc1 ; c1ccc(-c2ccc(-c3ccc(N(c4ccc5c(c4)oc4ccccc45)c4ccc5ccc6ccccc6c5c4)cc3)c3c2sc2ccccc23)cc1]

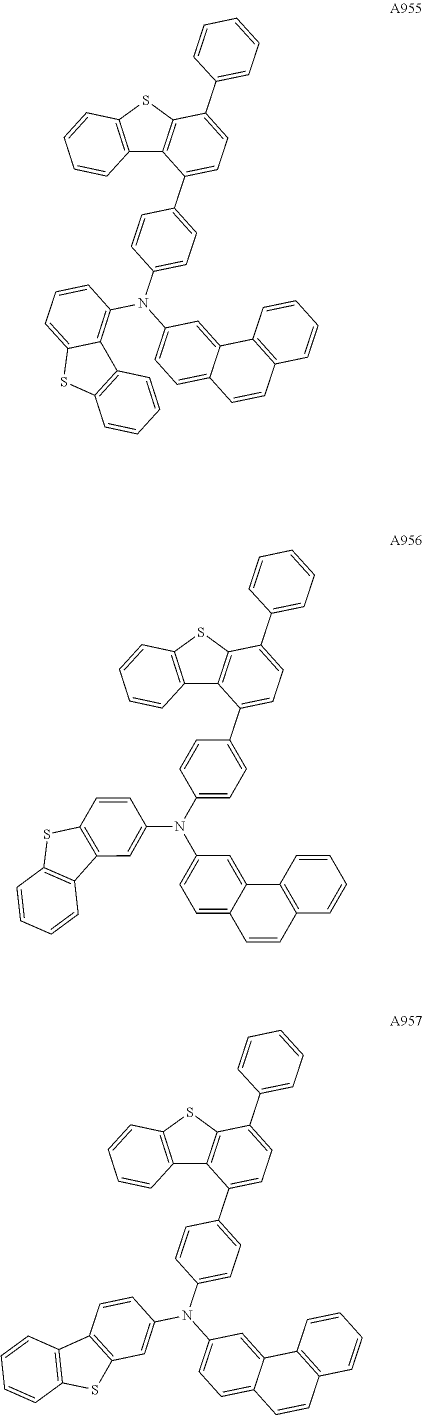 [SMILES: c1ccc(-c2ccc(-c3ccc(N(c4ccc5ccc6ccccc6c5c4)c4cccc5sc6ccccc6c45)cc3)c3c2sc2ccccc23)cc1 ; c1ccc(-c2ccc(-c3ccc(N(c4ccc5ccc6ccccc6c5c4)c4ccc5sc6ccccc6c5c4)cc3)c3c2sc2ccccc23)cc1 ; c1ccc(-c2ccc(-c3ccc(N(c4ccc5c(c4)sc4ccccc45)c4ccc5ccc6ccccc6c5c4)cc3)c3c2sc2ccccc23)cc1]

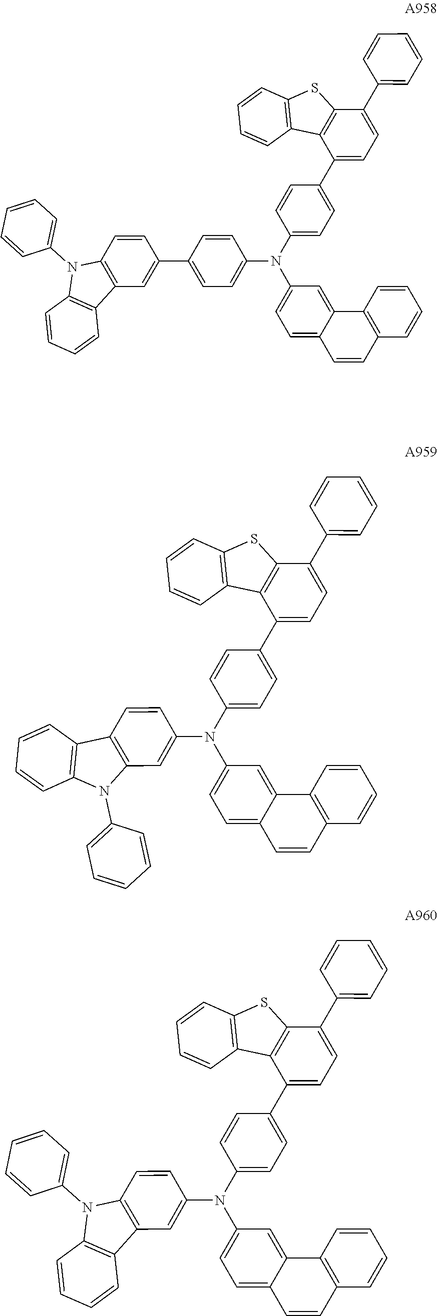 [SMILES: c1ccc(-c2ccc(-c3ccc(N(c4ccc(-c5ccc6c(c5)c5ccccc5n6-c5ccccc5)cc4)c4ccc5ccc6ccccc6c5c4)cc3)c3c2sc2ccccc23)cc1 ; c1ccc(-c2ccc(-c3ccc(N(c4ccc5ccc6ccccc6c5c4)c4ccc5c6ccccc6n(-c6ccccc6)c5c4)cc3)c3c2sc2ccccc23)cc1 ; c1ccc(-c2ccc(-c3ccc(N(c4ccc5ccc6ccccc6c5c4)c4ccc5c(c4)c4ccccc4n5-c4ccccc4)cc3)c3c2sc2ccccc23)cc1]

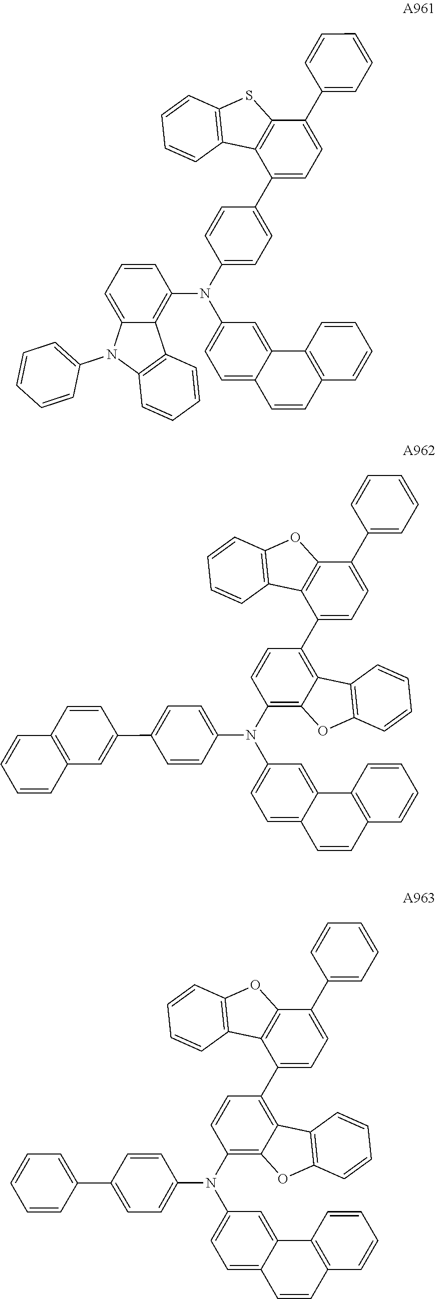 [SMILES: c1ccc(-c2ccc(-c3ccc(N(c4ccc5ccc6ccccc6c5c4)c4cccc5c4c4ccccc4n5-c4ccccc4)cc3)c3c2sc2ccccc23)cc1 ; c1ccc(-c2ccc(-c3ccc(N(c4ccc(-c5ccc6ccccc6c5)cc4)c4ccc5ccc6ccccc6c5c4)c4oc5ccccc5c34)c3c2oc2ccccc23)cc1 ; c1ccc(-c2ccc(N(c3ccc4ccc5ccccc5c4c3)c3ccc(-c4ccc(-c5ccccc5)c5oc6ccccc6c45)c4c3oc3ccccc34)cc2)cc1]

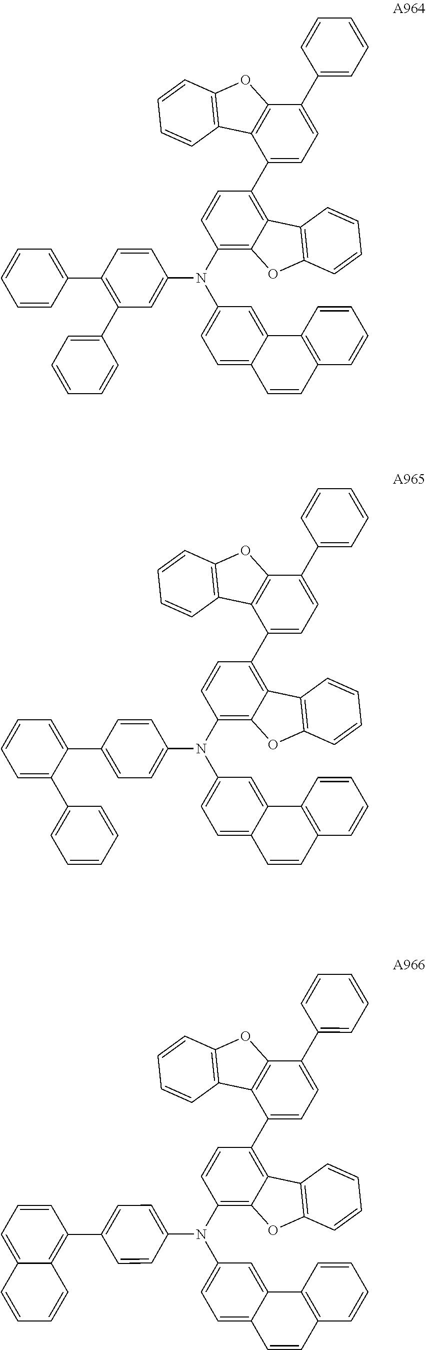 [SMILES: c1ccc(-c2ccc(N(c3ccc4ccc5ccccc5c4c3)c3ccc(-c4ccc(-c5ccccc5)c5oc6ccccc6c45)c4c3oc3ccccc34)cc2-c2ccccc2)cc1 ; c1ccc(-c2ccccc2-c2ccc(N(c3ccc4ccc5ccccc5c4c3)c3ccc(-c4ccc(-c5ccccc5)c5oc6ccccc6c45)c4c3oc3ccccc34)cc2)cc1 ; c1ccc(-c2ccc(-c3ccc(N(c4ccc(-c5cccc6ccccc56)cc4)c4ccc5ccc6ccccc6c5c4)c4oc5ccccc5c34)c3c2oc2ccccc23)cc1]

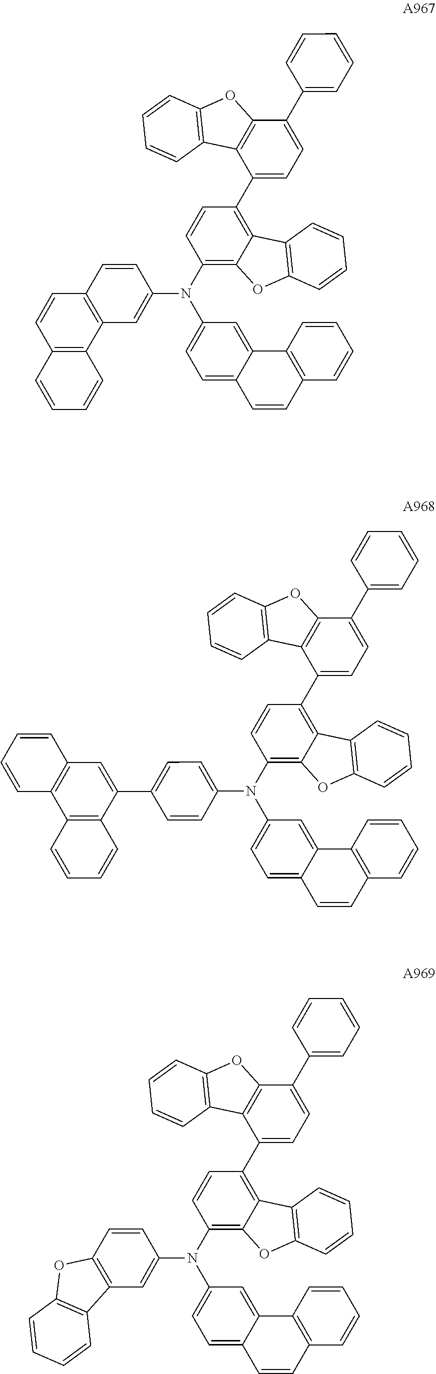 [SMILES: c1ccc(-c2ccc(-c3ccc(N(c4ccc5ccc6ccccc6c5c4)c4ccc5ccc6ccccc6c5c4)c4oc5ccccc5c34)c3c2oc2ccccc23)cc1 ; c1ccc(-c2ccc(-c3ccc(N(c4ccc(-c5cc6ccccc6c6ccccc56)cc4)c4ccc5ccc6ccccc6c5c4)c4oc5ccccc5c34)c3c2oc2ccccc23)cc1 ; c1ccc(-c2ccc(-c3ccc(N(c4ccc5ccc6ccccc6c5c4)c4ccc5oc6ccccc6c5c4)c4oc5ccccc5c34)c3c2oc2ccccc23)cc1]

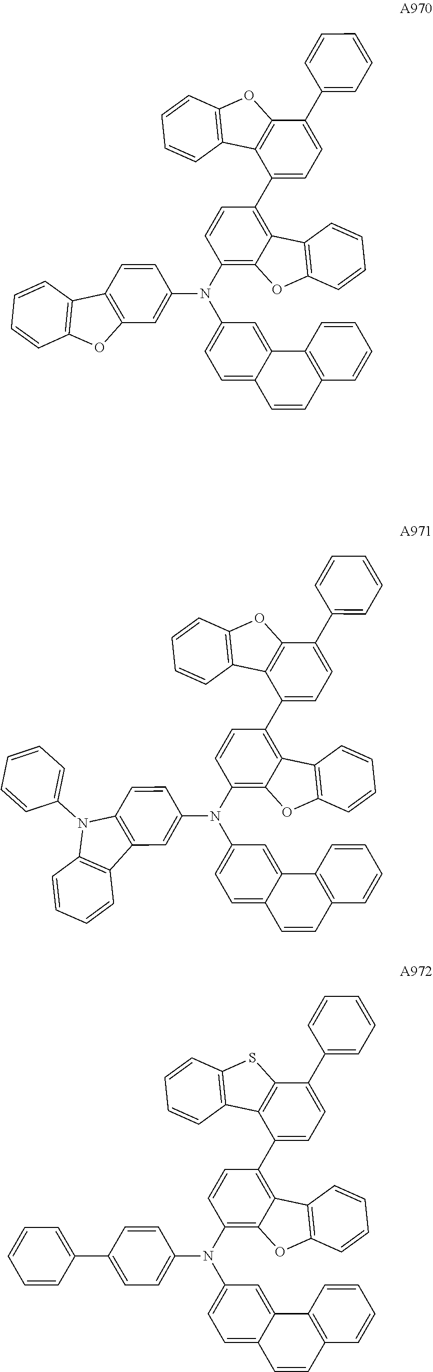 [SMILES: c1ccc(-c2ccc(-c3ccc(N(c4ccc5c(c4)oc4ccccc45)c4ccc5ccc6ccccc6c5c4)c4oc5ccccc5c34)c3c2oc2ccccc23)cc1 ; c1ccc(-c2ccc(-c3ccc(N(c4ccc5ccc6ccccc6c5c4)c4ccc5c(c4)c4ccccc4n5-c4ccccc4)c4oc5ccccc5c34)c3c2oc2ccccc23)cc1 ; c1ccc(-c2ccc(N(c3ccc4ccc5ccccc5c4c3)c3ccc(-c4ccc(-c5ccccc5)c5sc6ccccc6c45)c4c3oc3ccccc34)cc2)cc1]

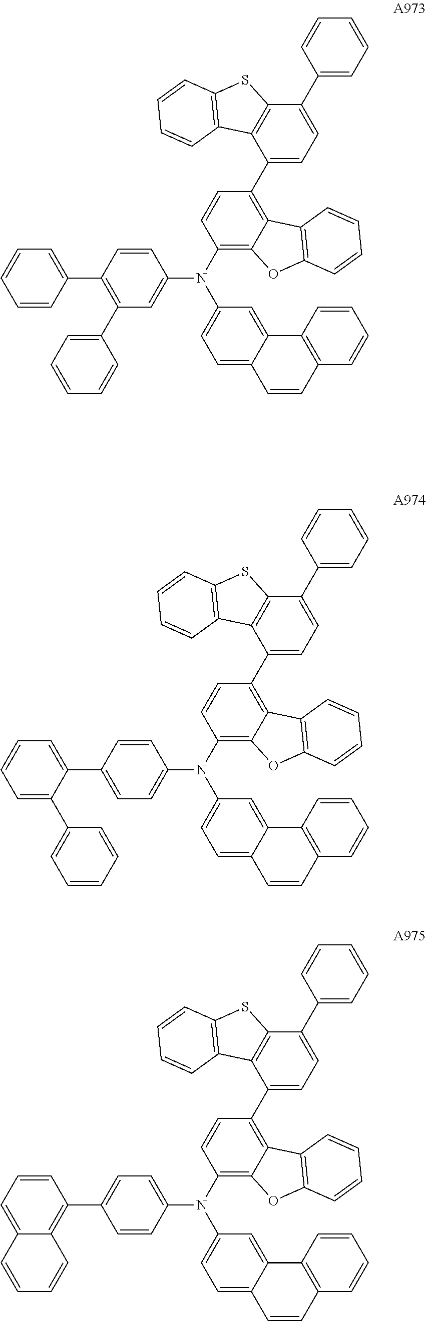 [SMILES: c1ccc(-c2ccc(N(c3ccc4ccc5ccccc5c4c3)c3ccc(-c4ccc(-c5ccccc5)c5sc6ccccc6c45)c4c3oc3ccccc34)cc2-c2ccccc2)cc1 ; c1ccc(-c2ccccc2-c2ccc(N(c3ccc4ccc5ccccc5c4c3)c3ccc(-c4ccc(-c5ccccc5)c5sc6ccccc6c45)c4c3oc3ccccc34)cc2)cc1 ; c1ccc(-c2ccc(-c3ccc(N(c4ccc(-c5cccc6ccccc56)cc4)c4ccc5ccc6ccccc6c5c4)c4oc5ccccc5c34)c3c2sc2ccccc23)cc1]

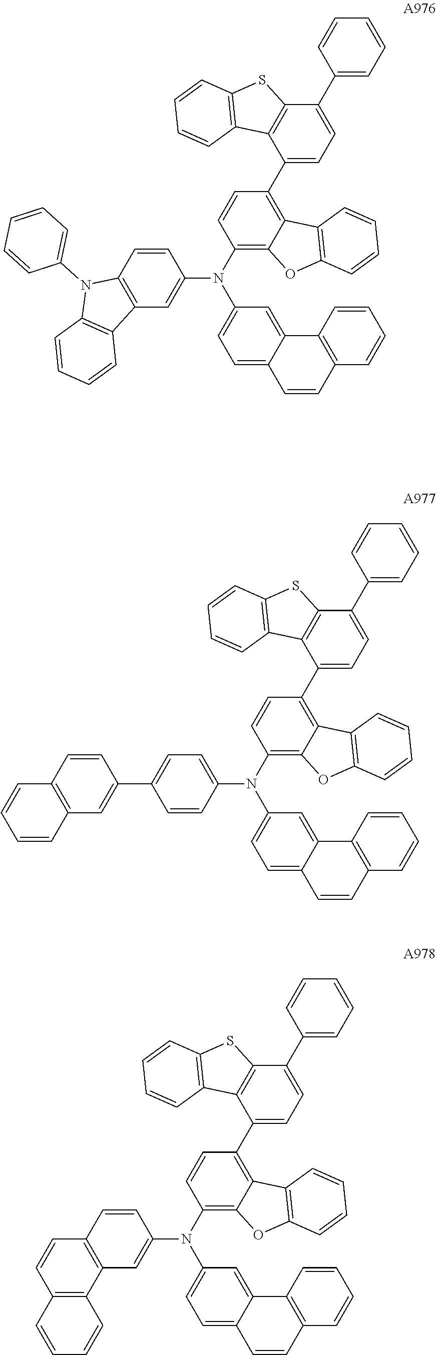 [SMILES: c1ccc(-c2ccc(-c3ccc(N(c4ccc5ccc6ccccc6c5c4)c4ccc5c(c4)c4ccccc4n5-c4ccccc4)c4oc5ccccc5c34)c3c2sc2ccccc23)cc1 ; c1ccc(-c2ccc(-c3ccc(N(c4ccc(-c5ccc6ccccc6c5)cc4)c4ccc5ccc6ccccc6c5c4)c4oc5ccccc5c34)c3c2sc2ccccc23)cc1 ; c1ccc(-c2ccc(-c3ccc(N(c4ccc5ccc6ccccc6c5c4)c4ccc5ccc6ccccc6c5c4)c4oc5ccccc5c34)c3c2sc2ccccc23)cc1]

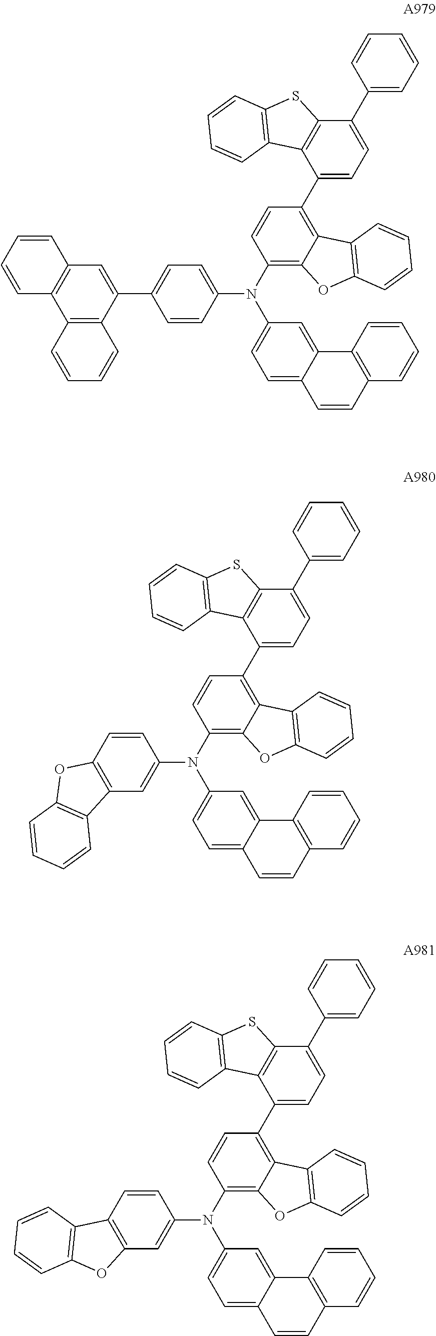 [SMILES: c1ccc(-c2ccc(-c3ccc(N(c4ccc(-c5cc6ccccc6c6ccccc56)cc4)c4ccc5ccc6ccccc6c5c4)c4oc5ccccc5c34)c3c2sc2ccccc23)cc1 ; c1ccc(-c2ccc(-c3ccc(N(c4ccc5ccc6ccccc6c5c4)c4ccc5oc6ccccc6c5c4)c4oc5ccccc5c34)c3c2sc2ccccc23)cc1 ; c1ccc(-c2ccc(-c3ccc(N(c4ccc5c(c4)oc4ccccc45)c4ccc5ccc6ccccc6c5c4)c4oc5ccccc5c34)c3c2sc2ccccc23)cc1]

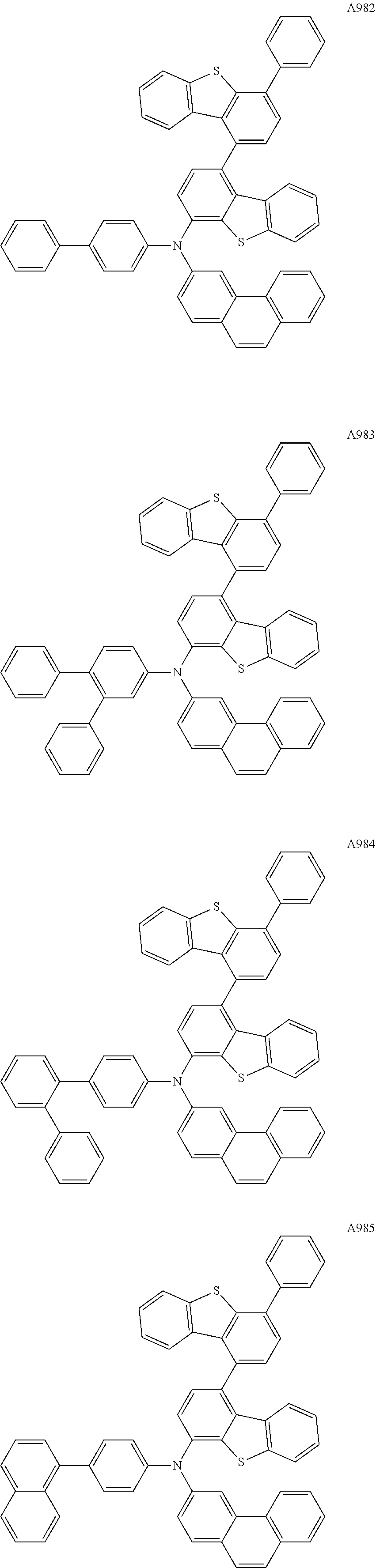 [SMILES: c1ccc(-c2ccc(N(c3ccc4ccc5ccccc5c4c3)c3ccc(-c4ccc(-c5ccccc5)c5sc6ccccc6c45)c4c3sc3ccccc34)cc2)cc1 ; c1ccc(-c2ccc(N(c3ccc4ccc5ccccc5c4c3)c3ccc(-c4ccc(-c5ccccc5)c5sc6ccccc6c45)c4c3sc3ccccc34)cc2-c2ccccc2)cc1 ; c1ccc(-c2ccccc2-c2ccc(N(c3ccc4ccc5ccccc5c4c3)c3ccc(-c4ccc(-c5ccccc5)c5sc6ccccc6c45)c4c3sc3ccccc34)cc2)cc1 ; c1ccc(-c2ccc(-c3ccc(N(c4ccc(-c5cccc6ccccc56)cc4)c4ccc5ccc6ccccc6c5c4)c4sc5ccccc5c34)c3c2sc2ccccc23)cc1]

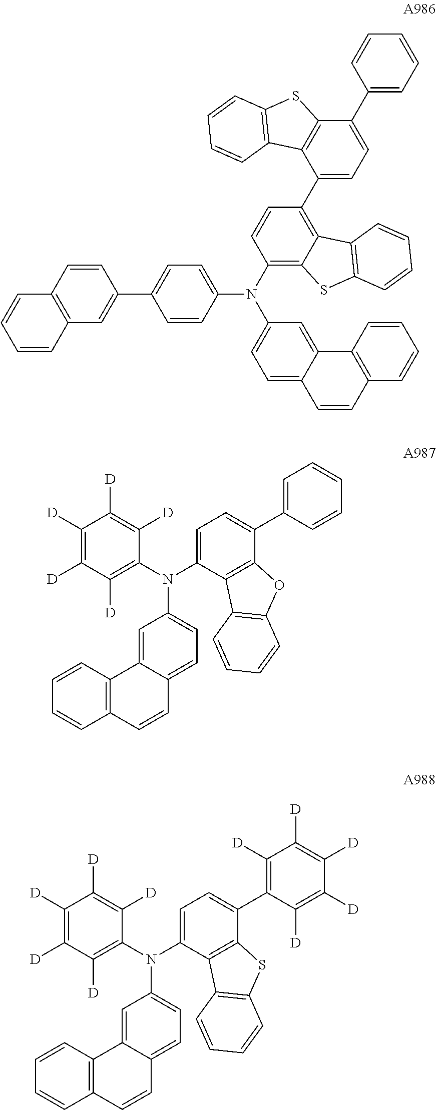 [SMILES: c1ccc(-c2ccc(-c3ccc(N(c4ccc(-c5ccc6ccccc6c5)cc4)c4ccc5ccc6ccccc6c5c4)c4sc5ccccc5c34)c3c2sc2ccccc23)cc1 ; [2H]c1c([2H])c([2H])c(N(c2ccc3ccc4ccccc4c3c2)c2ccc(-c3ccccc3)c3oc4ccccc4c23)c([2H])c1[2H] ; [2H]c1c([2H])c([2H])c(-c2ccc(N(c3ccc4ccc5ccccc5c4c3)c3c([2H])c([2H])c([2H])c([2H])c3[2H])c3c2sc2ccccc23)c([2H])c1[2H]]

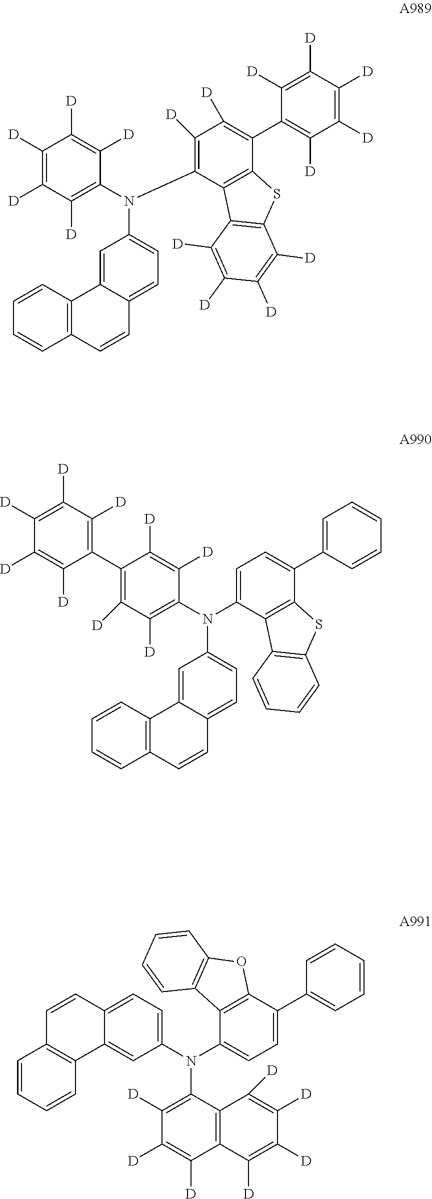 [SMILES: [2H]c1c([2H])c([2H])c(-c2c([2H])c([2H])c(N(c3ccc4ccc5ccccc5c4c3)c3c([2H])c([2H])c([2H])c([2H])c3[2H])c3c2sc2c([2H])c([2H])c([2H])c([2H])c23)c([2H])c1[2H] ; [2H]c1c([2H])c([2H])c(-c2c([2H])c([2H])c(N(c3ccc4ccc5ccccc5c4c3)c3ccc(-c4ccccc4)c4sc5ccccc5c34)c([2H])c2[2H])c([2H])c1[2H] ; [2H]c1c([2H])c([2H])c2c(N(c3ccc4ccc5ccccc5c4c3)c3ccc(-c4ccccc4)c4oc5ccccc5c34)c([2H])c([2H])c([2H])c2c1[2H]]

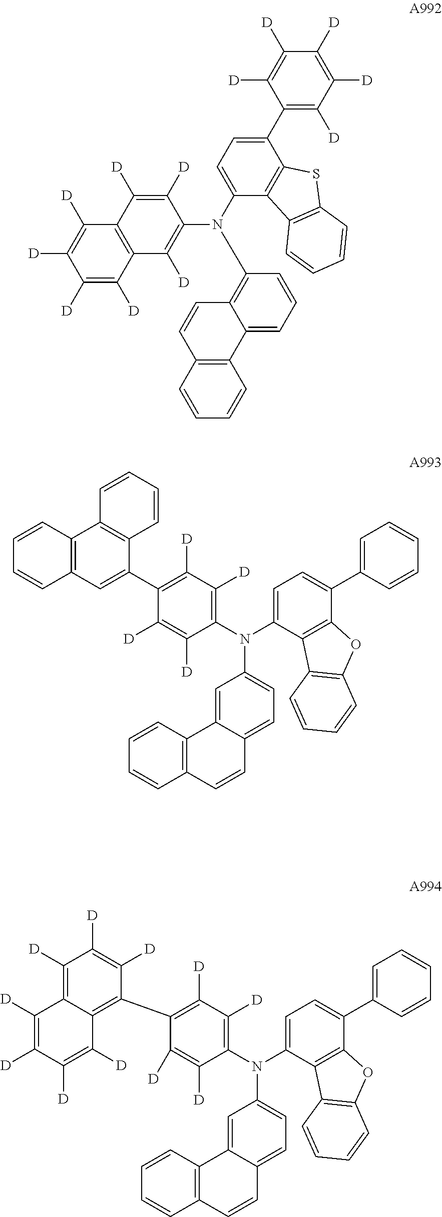 [SMILES: [2H]c1c([2H])c([2H])c(-c2ccc(N(c3c([2H])c([2H])c4c([2H])c([2H])c([2H])c([2H])c4c3[2H])c3cccc4c3ccc3ccccc34)c3c2sc2ccccc23)c([2H])c1[2H] ; [2H]c1c([2H])c(N(c2ccc3ccc4ccccc4c3c2)c2ccc(-c3ccccc3)c3oc4ccccc4c23)c([2H])c([2H])c1-c1cc2ccccc2c2ccccc12 ; [2H]c1c([2H])c(N(c2ccc3ccc4ccccc4c3c2)c2ccc(-c3ccccc3)c3oc4ccccc4c23)c([2H])c([2H])c1-c1c([2H])c([2H])c([2H])c2c([2H])c([2H])c([2H])c([2H])c12]

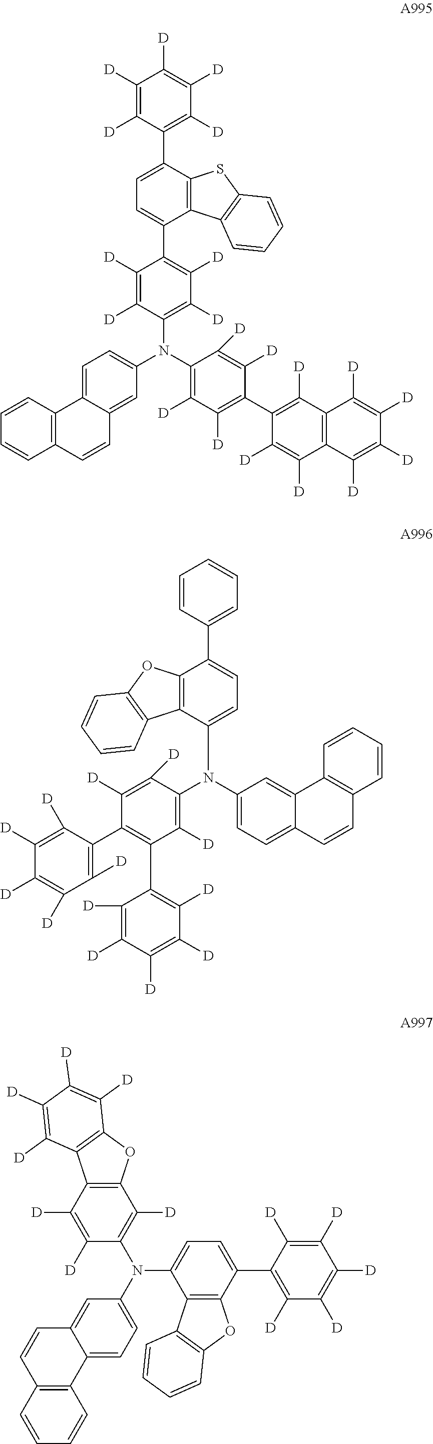

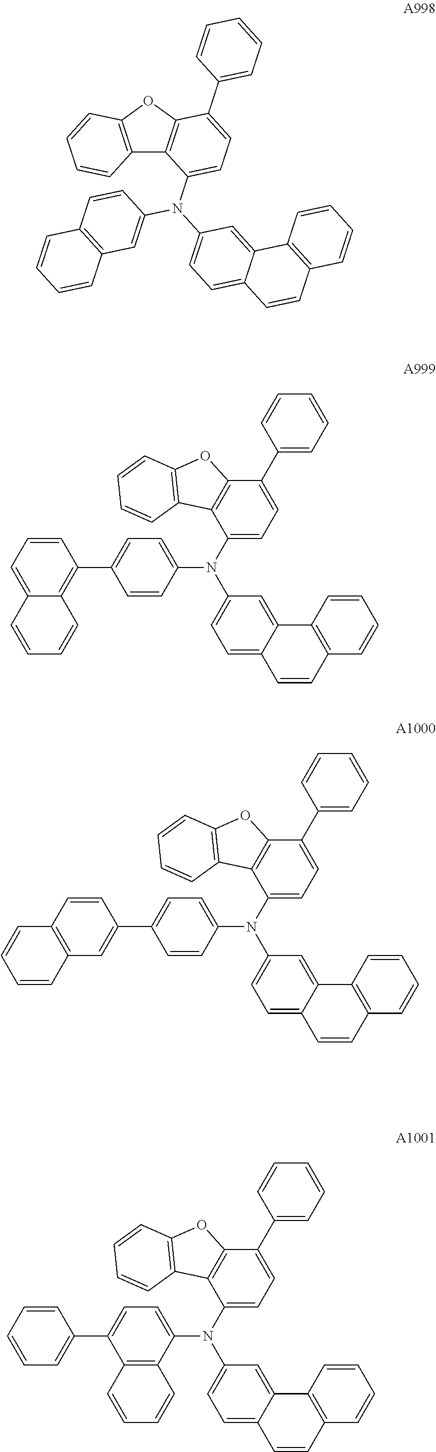 [SMILES: c1ccc(-c2ccc(N(c3ccc4ccccc4c3)c3ccc4ccc5ccccc5c4c3)c3c2oc2ccccc23)cc1 ; c1ccc(-c2ccc(N(c3ccc(-c4cccc5ccccc45)cc3)c3ccc4ccc5ccccc5c4c3)c3c2oc2ccccc23)cc1 ; c1ccc(-c2ccc(N(c3ccc(-c4ccc5ccccc5c4)cc3)c3ccc4ccc5ccccc5c4c3)c3c2oc2ccccc23)cc1 ; c1ccc(-c2ccc(N(c3ccc4ccc5ccccc5c4c3)c3ccc(-c4ccccc4)c4oc5ccccc5c34)c3ccccc23)cc1]

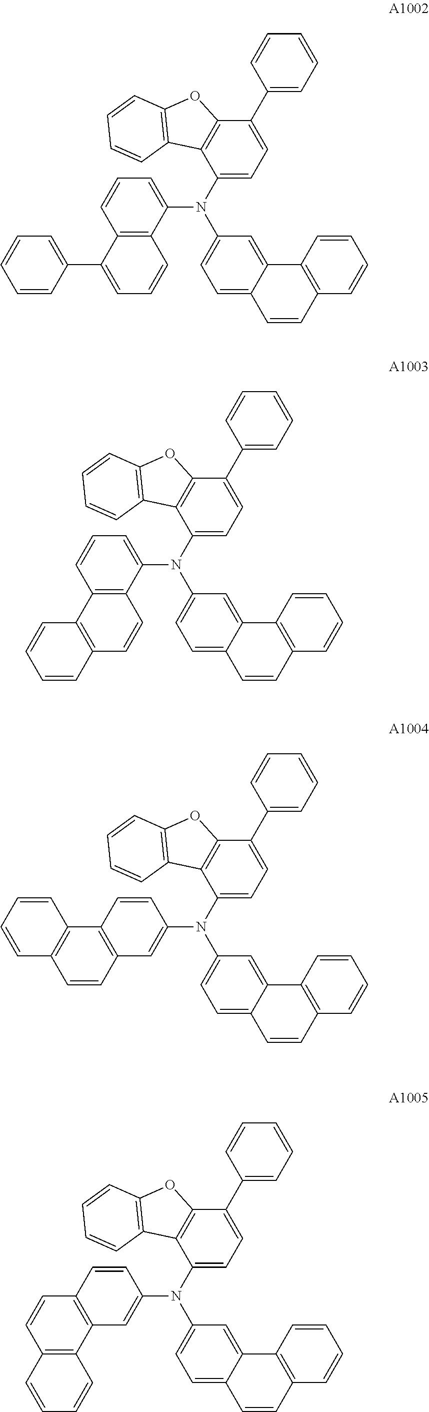 [SMILES: c1ccc(-c2cccc3c(N(c4ccc5ccc6ccccc6c5c4)c4ccc(-c5ccccc5)c5oc6ccccc6c45)cccc23)cc1 ; c1ccc(-c2ccc(N(c3ccc4ccc5ccccc5c4c3)c3cccc4c3ccc3ccccc34)c3c2oc2ccccc23)cc1 ; c1ccc(-c2ccc(N(c3ccc4c(ccc5ccccc54)c3)c3ccc4ccc5ccccc5c4c3)c3c2oc2ccccc23)cc1 ; c1ccc(-c2ccc(N(c3ccc4ccc5ccccc5c4c3)c3ccc4ccc5ccccc5c4c3)c3c2oc2ccccc23)cc1]

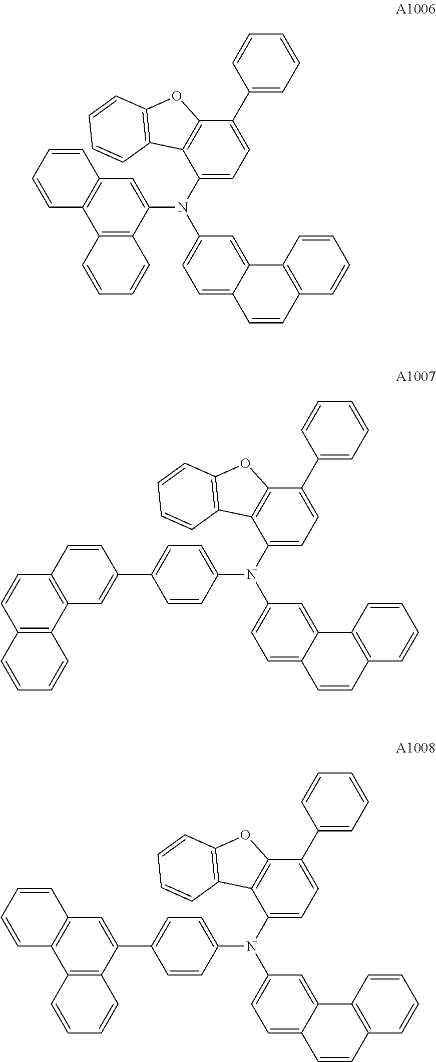 [SMILES: c1ccc(-c2ccc(N(c3ccc4ccc5ccccc5c4c3)c3cc4ccccc4c4ccccc34)c3c2oc2ccccc23)cc1 ; c1ccc(-c2ccc(N(c3ccc(-c4ccc5ccc6ccccc6c5c4)cc3)c3ccc4ccc5ccccc5c4c3)c3c2oc2ccccc23)cc1 ; c1ccc(-c2ccc(N(c3ccc(-c4cc5ccccc5c5ccccc45)cc3)c3ccc4ccc5ccccc5c4c3)c3c2oc2ccccc23)cc1]

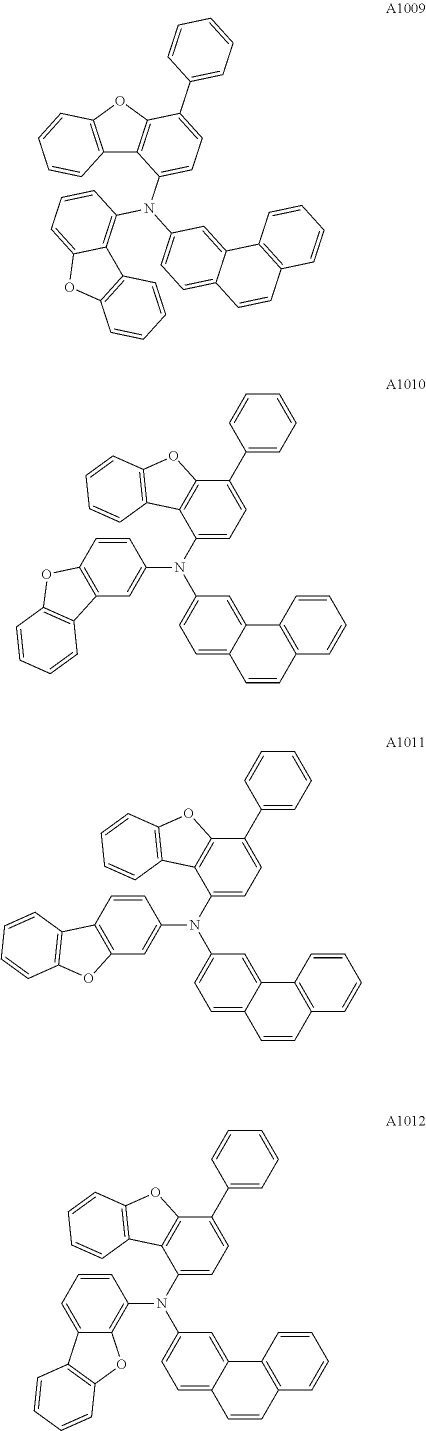 [SMILES: c1ccc(-c2ccc(N(c3ccc4ccc5ccccc5c4c3)c3cccc4oc5ccccc5c34)c3c2oc2ccccc23)cc1 ; c1ccc(-c2ccc(N(c3ccc4ccc5ccccc5c4c3)c3ccc4oc5ccccc5c4c3)c3c2oc2ccccc23)cc1 ; c1ccc(-c2ccc(N(c3ccc4c(c3)oc3ccccc34)c3ccc4ccc5ccccc5c4c3)c3c2oc2ccccc23)cc1 ; c1ccc(-c2ccc(N(c3ccc4ccc5ccccc5c4c3)c3cccc4c3oc3ccccc34)c3c2oc2ccccc23)cc1]

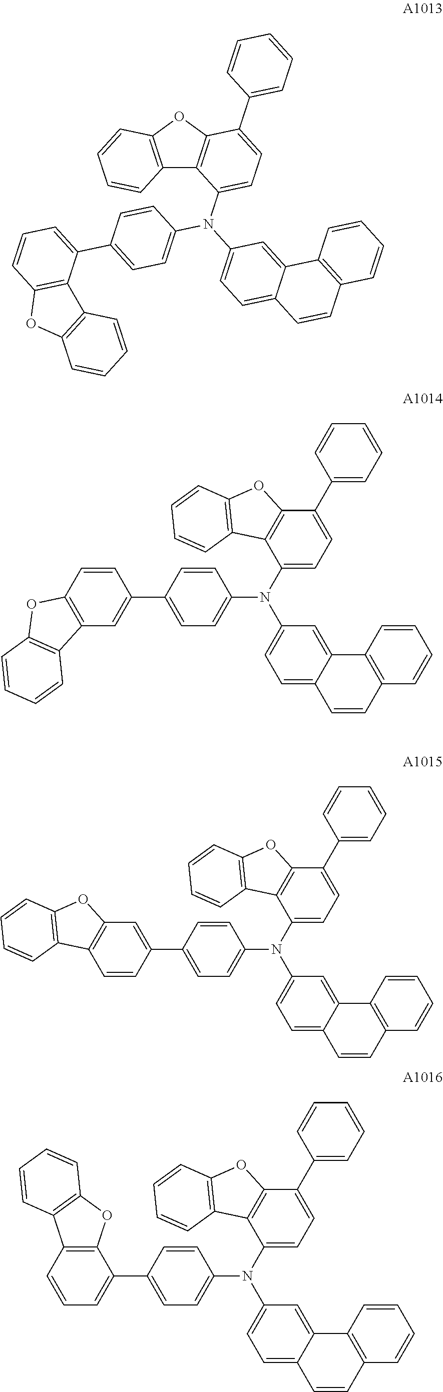

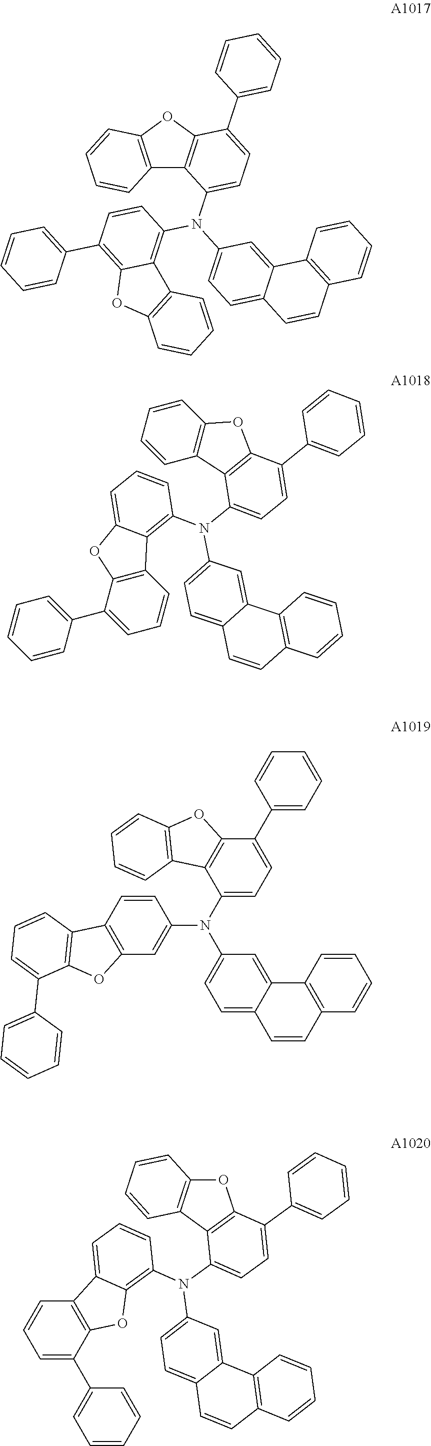

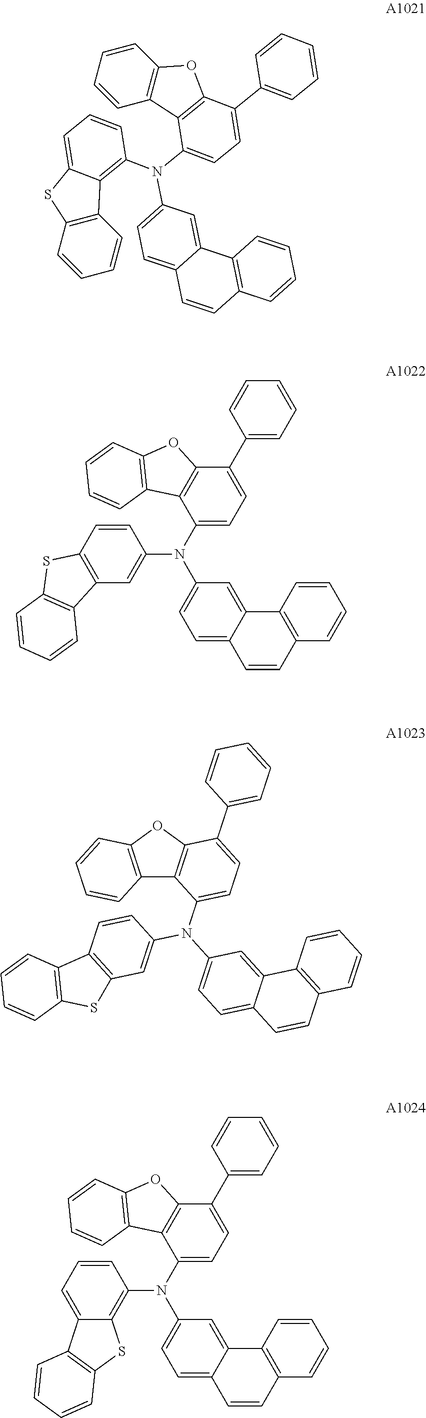 [SMILES: c1ccc(-c2ccc(N(c3ccc4ccc5ccccc5c4c3)c3cccc4sc5ccccc5c34)c3c2oc2ccccc23)cc1 ; c1ccc(-c2ccc(N(c3ccc4ccc5ccccc5c4c3)c3ccc4sc5ccccc5c4c3)c3c2oc2ccccc23)cc1 ; c1ccc(-c2ccc(N(c3ccc4c(c3)sc3ccccc34)c3ccc4ccc5ccccc5c4c3)c3c2oc2ccccc23)cc1 ; c1ccc(-c2ccc(N(c3ccc4ccc5ccccc5c4c3)c3cccc4c3sc3ccccc34)c3c2oc2ccccc23)cc1]

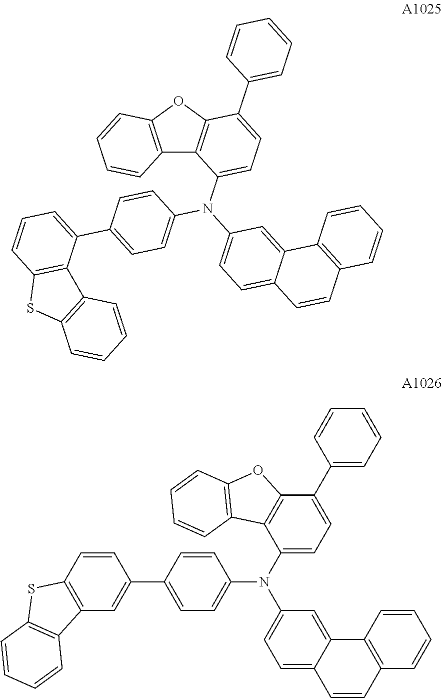 [SMILES: c1ccc(-c2ccc(N(c3ccc(-c4cccc5sc6ccccc6c45)cc3)c3ccc4ccc5ccccc5c4c3)c3c2oc2ccccc23)cc1 ; c1ccc(-c2ccc(N(c3ccc(-c4ccc5sc6ccccc6c5c4)cc3)c3ccc4ccc5ccccc5c4c3)c3c2oc2ccccc23)cc1]

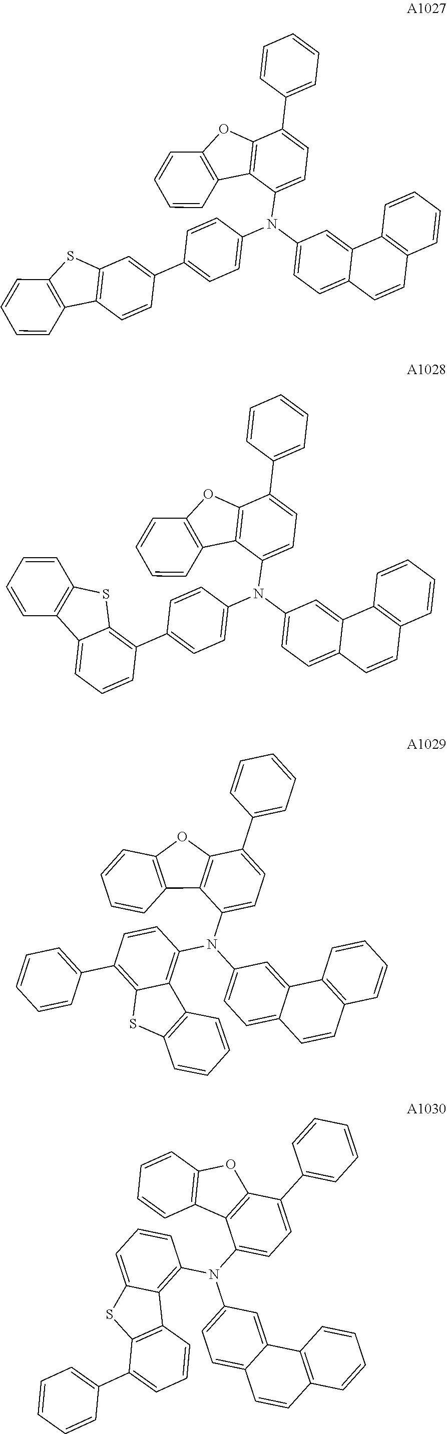 [SMILES: c1ccc(-c2ccc(N(c3ccc(-c4ccc5c(c4)sc4ccccc45)cc3)c3ccc4ccc5ccccc5c4c3)c3c2oc2ccccc23)cc1 ; c1ccc(-c2ccc(N(c3ccc(-c4cccc5c4sc4ccccc45)cc3)c3ccc4ccc5ccccc5c4c3)c3c2oc2ccccc23)cc1 ; c1ccc(-c2ccc(N(c3ccc4ccc5ccccc5c4c3)c3ccc(-c4ccccc4)c4sc5ccccc5c34)c3c2oc2ccccc23)cc1 ; c1ccc(-c2ccc(N(c3ccc4ccc5ccccc5c4c3)c3cccc4sc5c(-c6ccccc6)cccc5c34)c3c2oc2ccccc23)cc1]

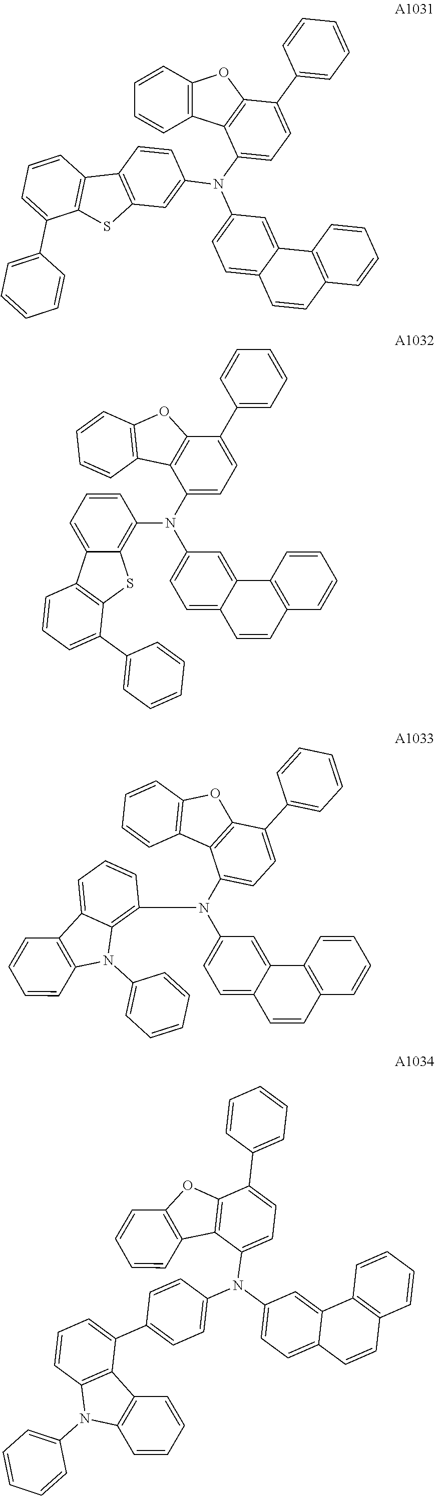

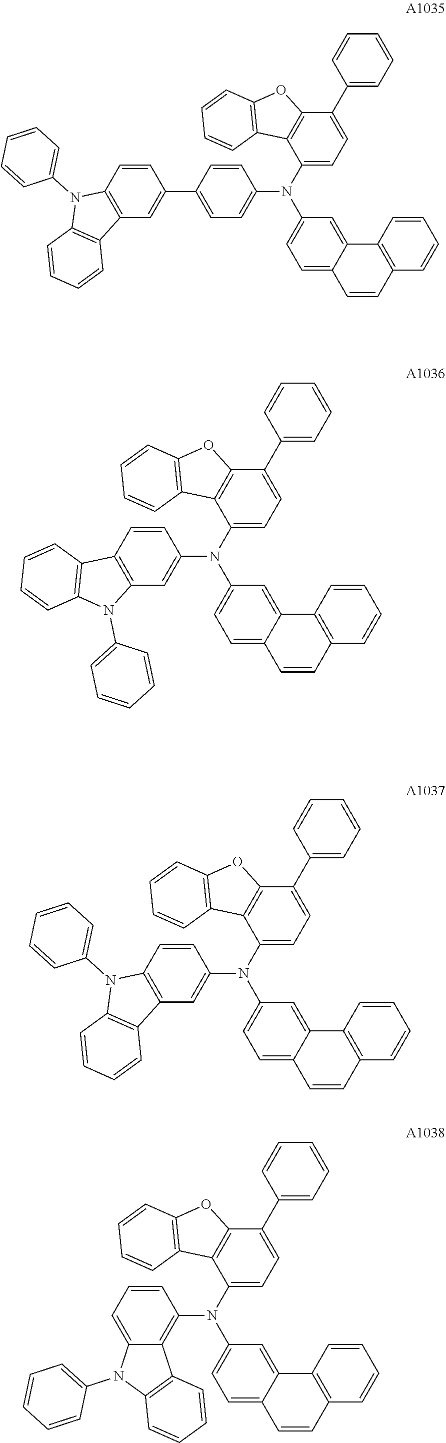 [SMILES: c1ccc(-c2ccc(N(c3ccc(-c4ccc5c(c4)c4ccccc4n5-c4ccccc4)cc3)c3ccc4ccc5ccccc5c4c3)c3c2oc2ccccc23)cc1 ; c1ccc(-c2ccc(N(c3ccc4ccc5ccccc5c4c3)c3ccc4c5ccccc5n(-c5ccccc5)c4c3)c3c2oc2ccccc23)cc1 ; c1ccc(-c2ccc(N(c3ccc4ccc5ccccc5c4c3)c3ccc4c(c3)c3ccccc3n4-c3ccccc3)c3c2oc2ccccc23)cc1 ; c1ccc(-c2ccc(N(c3ccc4ccc5ccccc5c4c3)c3cccc4c3c3ccccc3n4-c3ccccc3)c3c2oc2ccccc23)cc1]

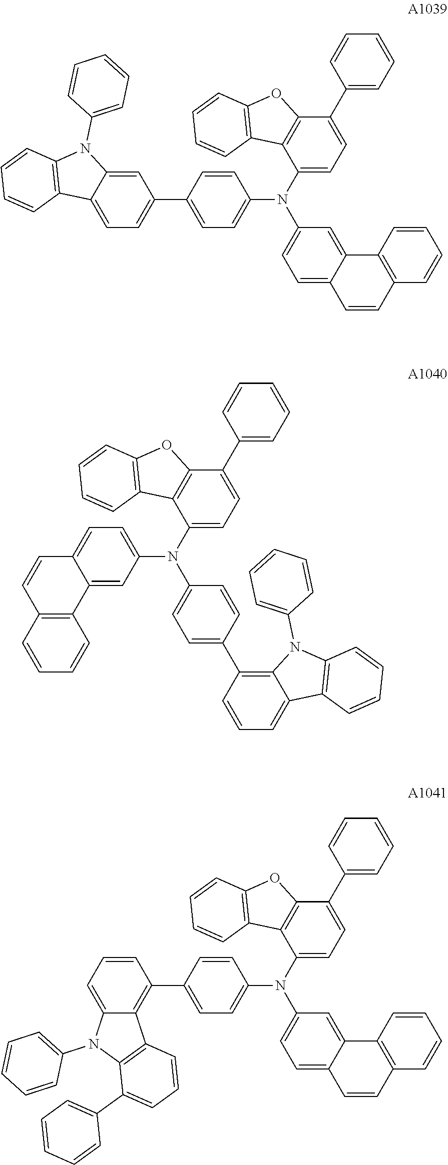 [SMILES: c1ccc(-c2ccc(N(c3ccc(-c4ccc5c6ccccc6n(-c6ccccc6)c5c4)cc3)c3ccc4ccc5ccccc5c4c3)c3c2oc2ccccc23)cc1 ; c1ccc(-c2ccc(N(c3ccc(-c4cccc5c6ccccc6n(-c6ccccc6)c45)cc3)c3ccc4ccc5ccccc5c4c3)c3c2oc2ccccc23)cc1 ; c1ccc(-c2ccc(N(c3ccc(-c4cccc5c4c4cccc(-c6ccccc6)c4n5-c4ccccc4)cc3)c3ccc4ccc5ccccc5c4c3)c3c2oc2ccccc23)cc1]

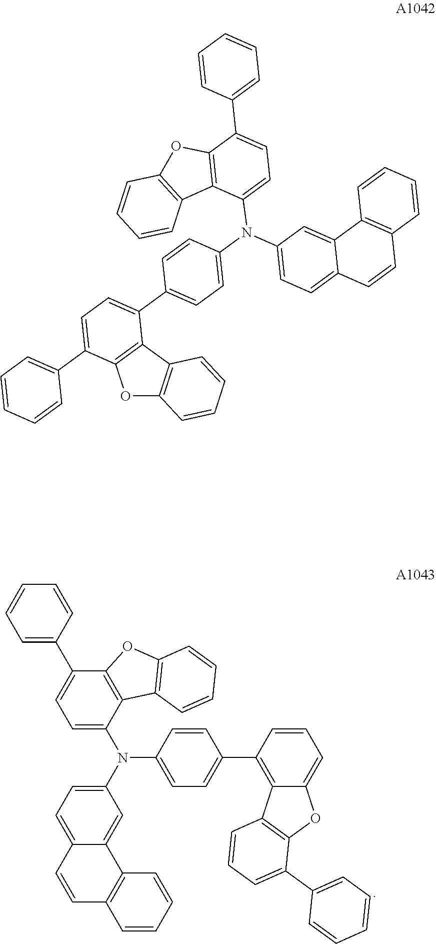 [SMILES: c1ccc(-c2ccc(-c3ccc(N(c4ccc5ccc6ccccc6c5c4)c4ccc(-c5ccccc5)c5oc6ccccc6c45)cc3)c3c2oc2ccccc23)cc1 ; c1ccc(-c2cccc3c2oc2cccc(-c4ccc(N(c5ccc6ccc7ccccc7c6c5)c5ccc(-c6ccccc6)c6oc7ccccc7c56)cc4)c23)cc1]

In Compound Group 1, D is a deuterium atom.

The amine compound according to an embodiment, represented by Formula 1, may include first, second, and third substituents bonded to an amine group. The first substituent may include a substituted dibenzofuran group or a substituted dibenzothiophene group. In Formula 1, a fused tricyclic ring containing X₁ may correspond to the first substituent. The second substituent may include a substituted or unsubstituted phenanthryl group. In Formula 1, Ar₂ may be a group represented by Formula 2 and may correspond to the second substituent. The third substituent may include a substituted or unsubstituted aryl group having 6 to 30 ring-forming carbon atoms or a substitute or unsubstituted heteroaryl group having 2 to 30 ring-forming carbon atoms. In Formula 1, Ar₃ may correspond to the third substituent.

In the specification, the carbon atoms of a dibenzofuran group and the carbon atoms of a dibenzothiophene group are numbered as follows:

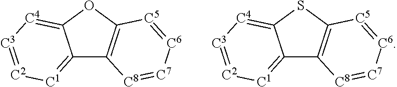

In the first substituent, the substituted dibenzofuran group (or substituted dibenzothiophene group), carbon atom number 1 (C¹) among the ring-forming atoms may be directly bonded to an amine group. A substituted or unsubstituted aryl group or a substituted or unsubstituted heteroaryl group (corresponding to Ar₁ in Formula 1) may be bonded to carbon atom number 4 (C⁴). Therefore, in the amine compound according to an embodiment, a conjugation system is expanded to the aryl group (or the heteroaryl group) and carbon atom number 3 (C³), which are bonded to the carbon atom number 4 (C⁴), and thus resonance energy may be stabilized, and excellent material stability may be exhibited.

A phenanthryl group, which is the second substituent, may include three-ring groups constituting the phenanthryl group, and the inwardly disposed ring group among the three-ring groups (the ring group including Rₐ₇ in Formula 2) may be directly bonded to an amine group. In Formula 2, the ring group disposed in the center among the three ring groups (the ring group including Rₐ₁ and Rₐ₂ in Formula 2) may not be bonded to an amine group. Compared to a compound in which the ring group disposed in the center is directly bonded to an amine group, the amine compound according to an embodiment, in which the inwardly disposed ring group is directly bonded to an amine group, may prevent steric distortion, and thus excellent thermal stability may be exhibited. Therefore, the amine compound according to an embodiment may contribute to a decrease in driving voltage, and improvements in efficiency and lifespan of the light-emitting element ED.

A hole transport region HTR may be provided on a first electrode EL1. The hole transport region HTR may include at least one of a hole injection layer HIL, a hole transport layer HTL, a buffer layer (not shown), an emission auxiliary layer (not shown), and an electron blocking layer EBL. A thickness of the hole transport region HTR may be, for example, in a range of about 50 Å to about 15,000 Å.

The hole transport region HTR may have a structure consisting of a layer consisting of a single material, a structure consisting of a layer including different materials, or a structure including multiple layers including different materials.

In embodiments, the hole transport region HTR may have a single-layered structure of a hole injection layer HIL or a hole transport layer HTL, or may have a structure of a single layer made of a hole injection material and a hole transport material. In embodiments, the hole transport region HTR may have a structure of a single layer made of different materials, or may have a structure of a hole injection layer HIL/hole transport layer HTL, a hole injection layer HIL/hole transport layer HTL/buffer layer (not shown), a hole injection layer HIL/buffer layer (not shown), a hole transport layer HTL/buffer layer (not shown), or a hole injection layer HIL/hole transport layer HTL/electron blocking layer EBL, in which the layers of each structure are stacked in its respective stated order from the first electrode EL1, but embodiments are not limited thereto.

The hole transport region HTR may be formed by using various methods such as a vacuum deposition method, a spin coating method, a casting method, the Langmuir-Blodgett (LB) method, an inkjet printing method, a laser printing method, and a laser induced thermal imaging (LITI) method.

The hole transport region HTR may further include, in addition to the amine compound according to an embodiment, additional hole transport compounds, which will be described below.

In the light-emitting element ED according to an embodiment, the hole transport region HTR may include a compound represented by Formula H-1:

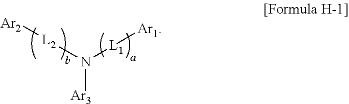

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. In Formula H-1, a and b may each independently be an integer from 0 to 10. When a or b is 2 or greater, multiple Li groups or multiple L₂ groups may each independently be a substituted or unsubstituted arylene group having 6 to 30 ring-forming carbon atoms or a substituted or unsubstituted heteroarylene group having 2 to 30 ring-forming carbon atoms.

In Formula H-1, An and Ar₂ may each independently be a substituted or unsubstituted aryl group having 6 to 30 ring-forming carbon atoms or a substituted or unsubstituted heteroaryl group having 2 to 30 ring-forming carbon atoms. In Formula H-1, Ar₃ may be a substituted or unsubstituted aryl group having 6 to 30 ring-forming carbon atoms or a substituted or unsubstituted heteroaryl group having 2 to 30 ring-forming carbon atoms.

In an embodiment, the compound represented by Formula H-1 may be a monoamine compound. In another embodiment, the compound represented by Formula H-1 may be a diamine compound in which at least one of Ar₁ to Ar₃ includes an amine group as a substituent.

In an embodiment, the compound represented by Formula H-1 may be a carbazole-based compound in which at least one of Ar₁ and Ar₂ includes a substituted or unsubstituted carbazole group, or may be a fluorene-based compound in which at least one of An and Ar₂ includes a substituted or unsubstituted fluorene group.

The compound represented by Formula H-1 may be any compound selected from Compound Group H. However, the compounds listed in Compound Group H are only examples, and a compound represented by Formula H-1 is not limited to Compound Group H:

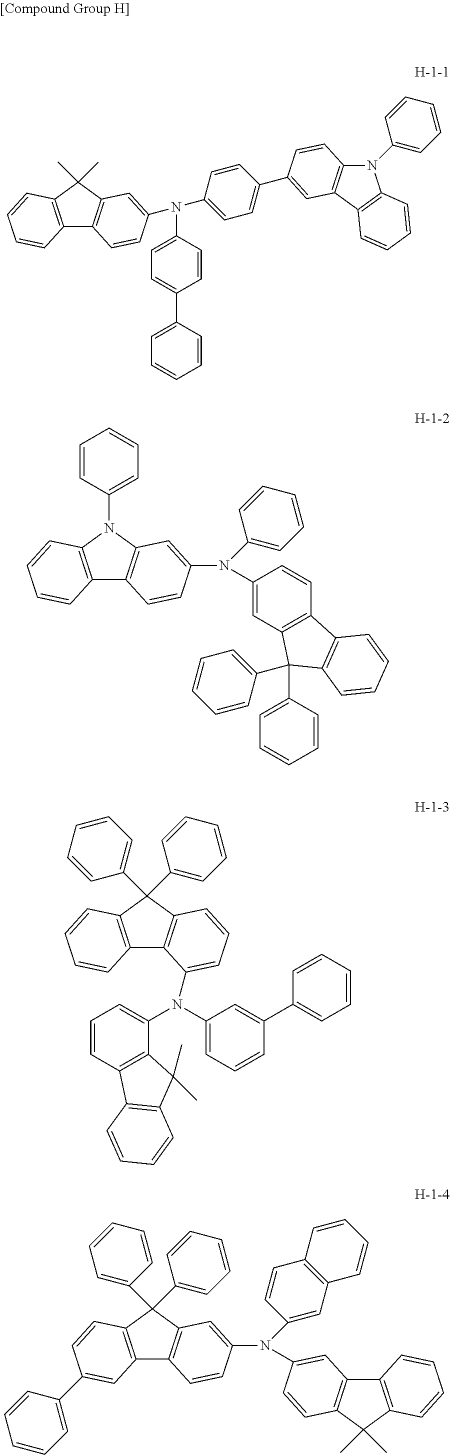 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

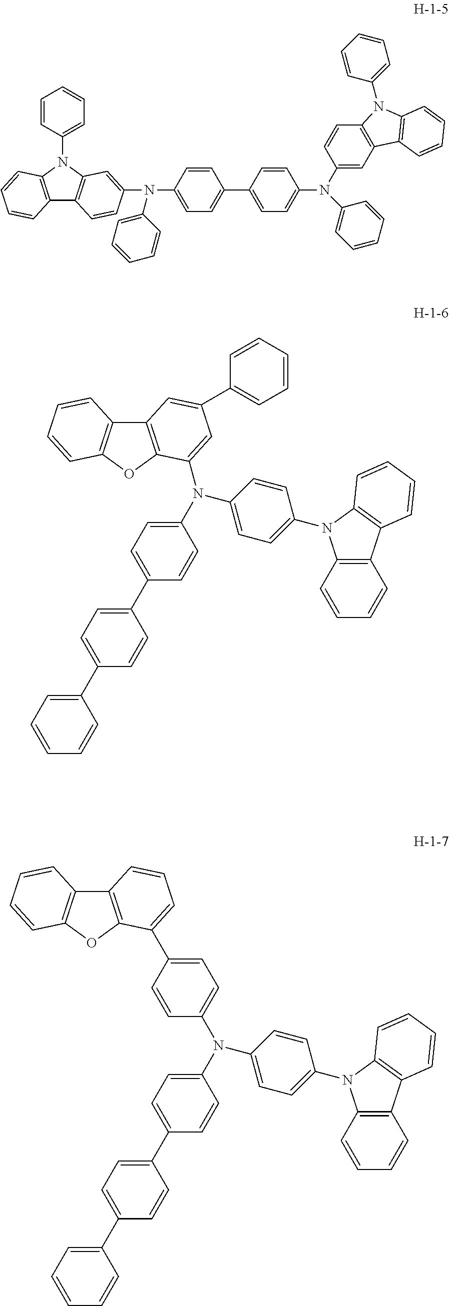 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

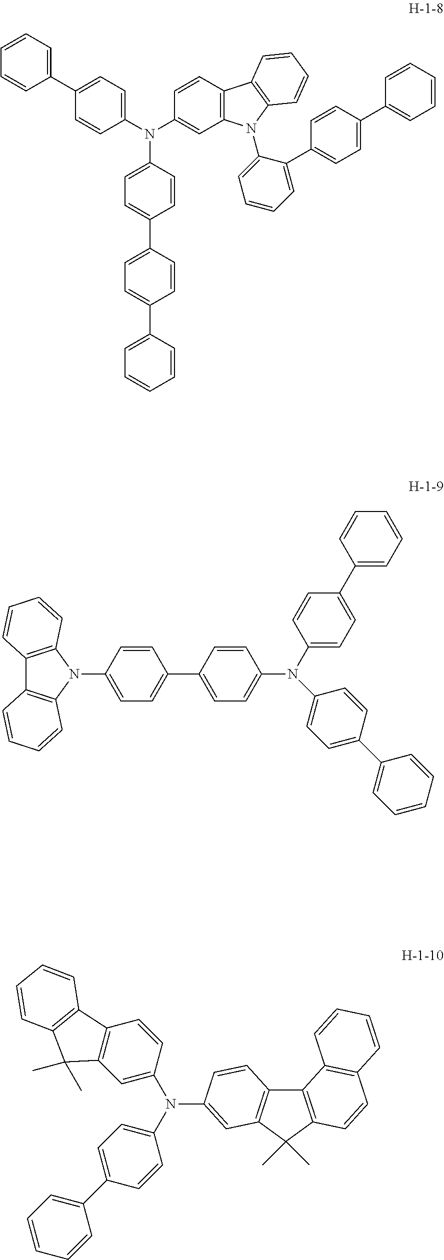 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

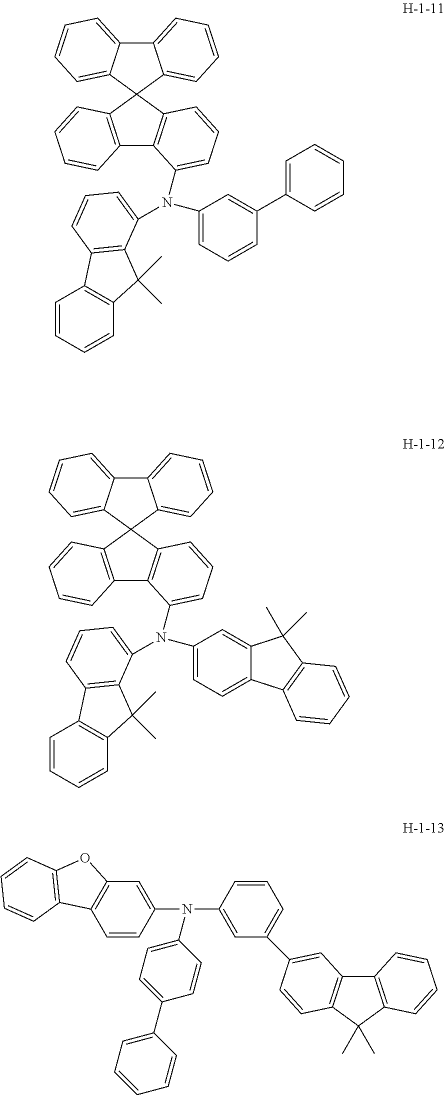 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

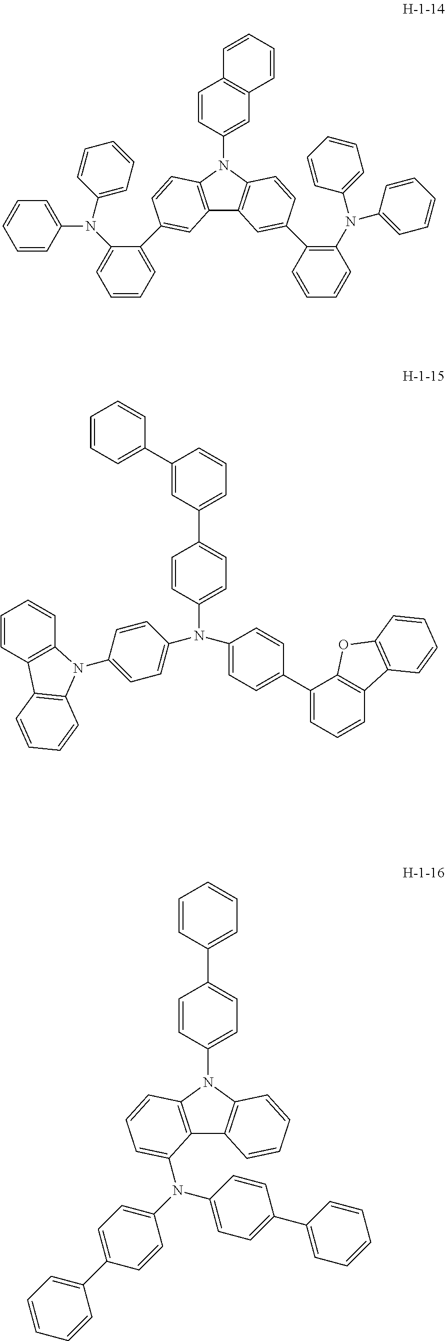 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

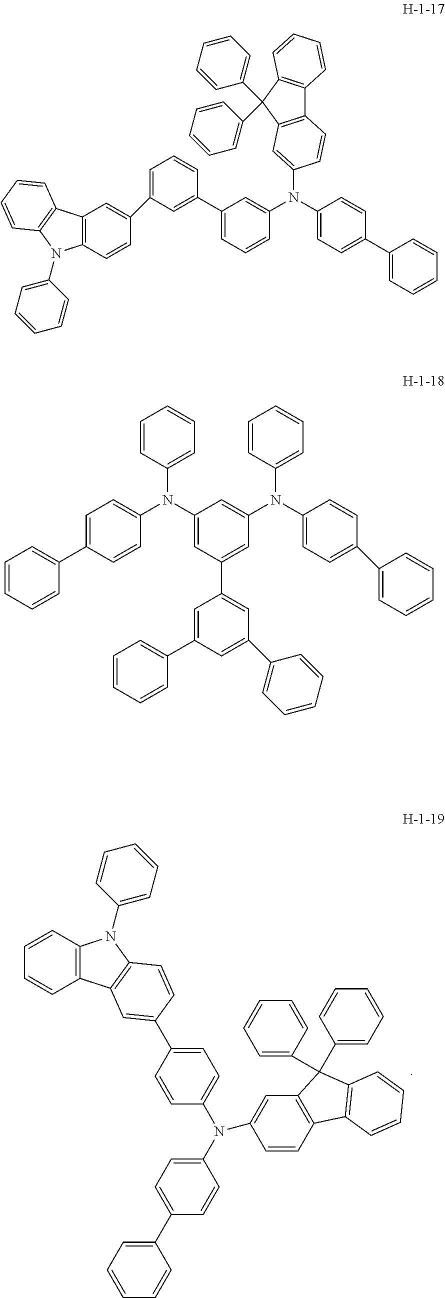 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

The hole transport region HTR may include a phthalocyanine compound such as copper phthalocyanine, N¹,N¹′-([1,1′-biphenyl]-4,4′-diyl)bis(N-phenyl-N4,N4-di-m-tolylbenzene-1,4-diamine)(DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl]benzenamine] (TAPC), 4,4′-bis[N,N′-(3-tolyl)amino]-3,3′-dimethylbiphenyl (HMTPD), 1,3-bis(N-carbazolyl)benzene (mCP), etc.

In an embodiment, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, and an electron blocking layer EBL.

A thickness of the hole transport region HTR may be in a range of about 100 Å to about 10,000 Å. For example, the thickness of the hole transport region HTR may be in a range of about 100 Å to about 5,000 Å. When the hole transport region HTR includes a hole injection layer HIL, the hole injection layer HIL may have, for example, a thickness in a range of about 30 Å to about 1,000 Å. When the hole transport region HTR includes a hole transport layer HTL, the hole transport layer HTL may have a thickness in a range of about 30 Å to about 1,000 Å. When the hole transport region HTR includes an electron blocking layer EBL, the electron blocking layer EBL may have a thickness in a range of about 10 Å to about 1,000 Å. If the thicknesses of the hole transport region HTR, the hole injection layer HIL, the hole transport layer HTL, and the electron blocking layer EBL satisfy the above-described ranges, satisfactory hole transport properties may be achieved without a substantial increase in driving voltage.

The hole transport region HTR may further include a charge generating material to increase conductivity, in addition to the above-described materials. The charge generating material may be dispersed uniformly or non-uniformly in the hole transport region HTR. The charge generating material may be, for example, a p-dopant. The p-dopant may include at least one of a metal halide, a quinone derivative, a metal oxide, or a cyano group-containing compound, but embodiments are not limited thereto. For example, the p-dopant may include a metal halide such as CuI or RbI, a quinone derivative such as tetracyanoquinodimethane (TCNQ) or 2,3,5,6-tetrafluoro-7,7′8,8-tetracyanoquinodimethane (F4-TCNQ), a metal oxide such as tungsten oxide or molybdenum oxide, a cyano group-containing compound such as dipyrazino[2,3-f: 2′,3′-h]quinoxaline-2,3,6,7,10,11-hexacarbonitrile (HATCN) or 4-[[2,3-bis [cyano-(4-cyano-2,3,5,6-tetrafluorophenyl)methylidene]cyclopropylidene]-cyanomethyl]-2,3,5,6-tetrafluorobenzonitrile (NDP9), etc., but embodiments are not limited thereto.

As described above, the hole transport region HTR may further include at least one of a buffer layer (not shown) and an electron blocking layer EBL, in addition to a hole injection layer HIL and a hole transport layer HTL. The buffer layer (not shown) may compensate for a resonance distance according to a wavelength of light emitted from an emission layer EML and may thus increase light emission efficiency. A material that may be included in the hole transport region HTR may be used as a material in the buffer layer (not shown). The electron blocking layer EBL may prevent the injection of electrons from an electron transport region ETR to the hole transport region HTR.

The emission layer EML may be provided on the hole transport region HTR. The emission layer EML may have a thickness in a range of about 100 Å to about 1,000 Å. For example, the emission layer EML may have a thickness in a range of about 100 Å to about 300 Å. The emission layer EML may have a structure consisting of a layer consisting of a single material, a structure consisting of a layer including different materials, or a structure including multiple layers including different materials.

In the light-emitting element ED, the emission layer EML may include an anthracene derivative, a pyrene derivative, a fluoranthene derivative, a chrysene derivative, a dihydrobenzanthracene derivative, or a triphenylene derivative. For example, the emission layer EML may include an anthracene derivative or a pyrene derivative.

In the light-emitting elements ED according to embodiments as shown in each of FIGS. 3 to 6, the emission layer EML may further include a host and a dopant of the related art.

In an embodiment, the emission layer EML may include a compound represented by Formula E-1. The compound represented by Formula E-1 may be used as a fluorescent host material:

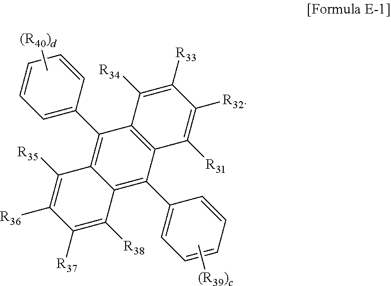

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. For example, R₃₁ to R₄₀ may be bonded to an adjacent group to form a saturated hydrocarbon ring, an unsaturated hydrocarbon ring, a saturated heterocycle, or an unsaturated heterocycle.

In Formula E-1, c and d may each independently be an integer from 0 to 5.

In an embodiment, the compound represented by Formula E-1 may be any compound selected from Compound El to Compound E19:

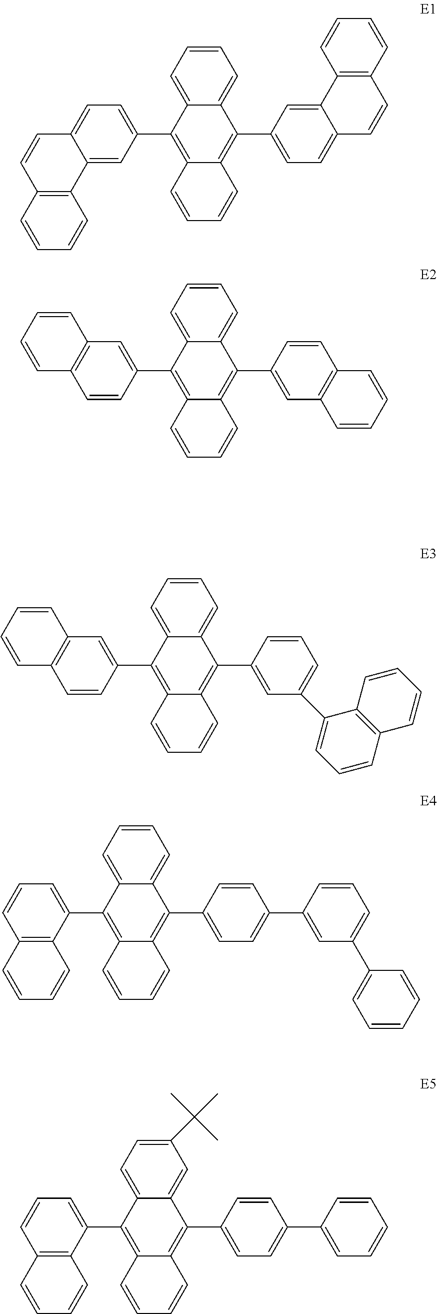

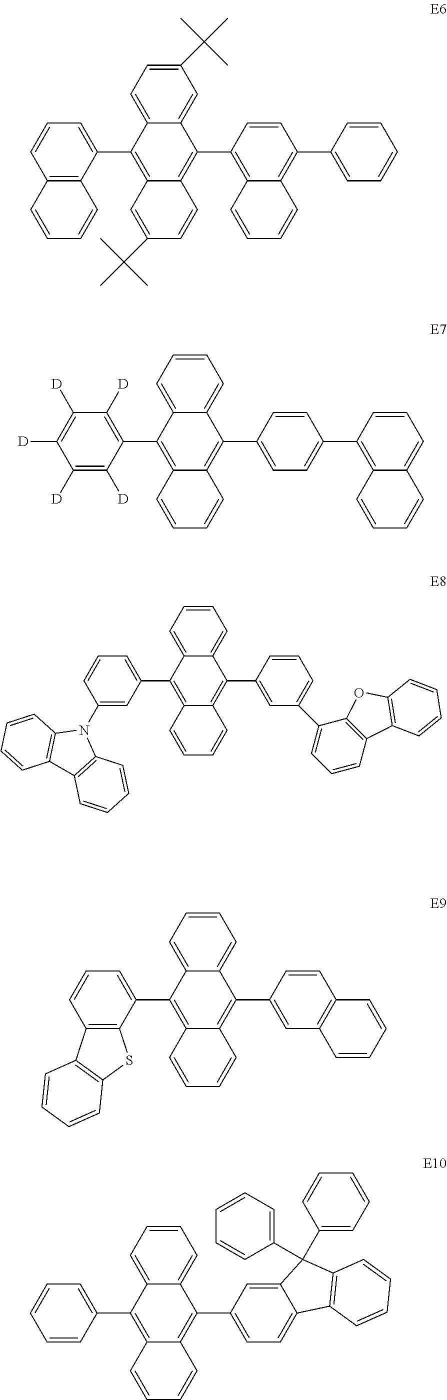

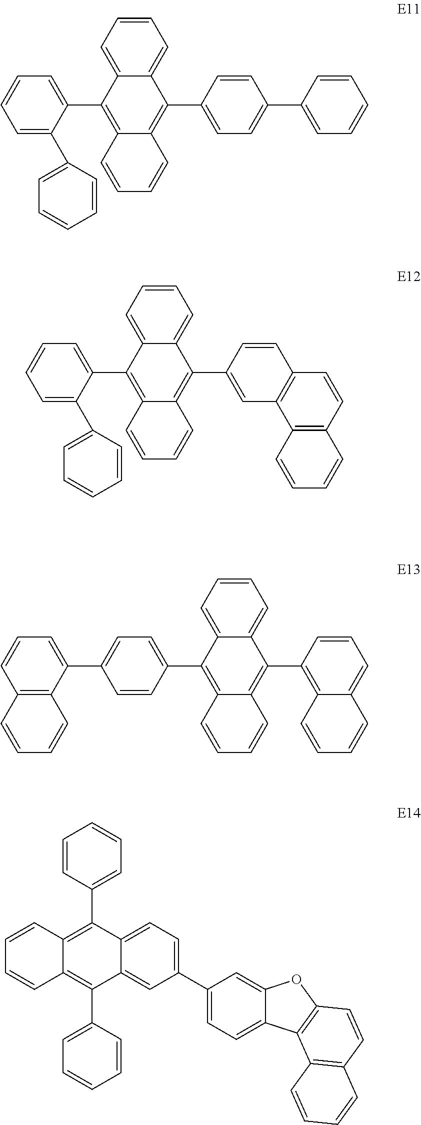

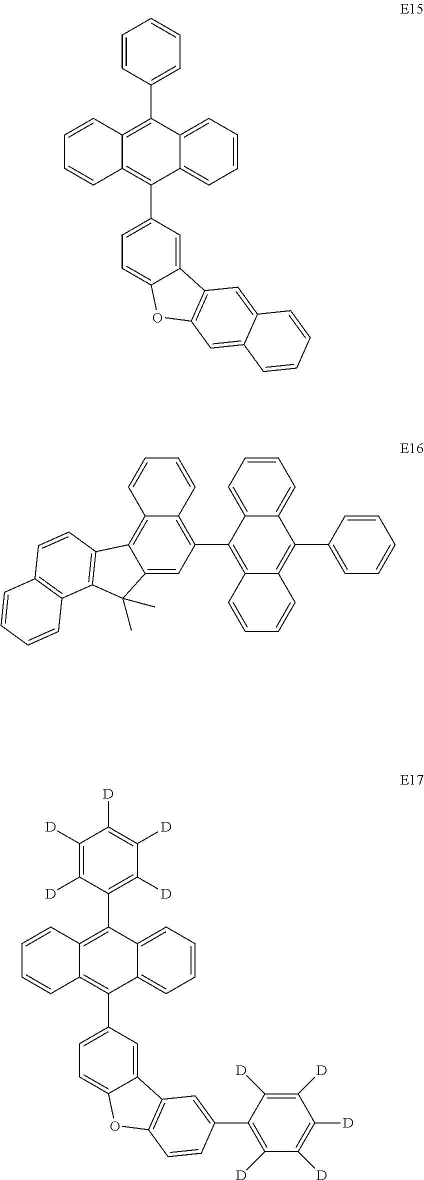

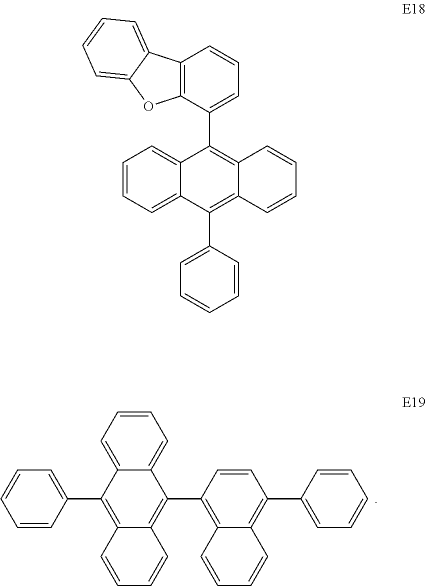

In an embodiment, the emission layer EML may include a compound represented by Formula E-2a or Formula E-2b. The compound represented by Formula E-2a or Formula E-2b may be used as a host material for a phosphorescent element.

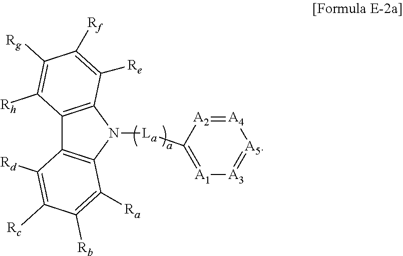

In Formula E-2a, a may be an integer from 0 to 10; and Lₐ may be a direct linkage, a substituted or unsubstituted arylene group having 6 to 30 ring-forming carbon atoms, or a substituted or unsubstituted heteroarylene group having 2 to 30 ring-forming carbon atoms. When a is 2 or greater, multiple Lₐ groups may each independently be a substituted or unsubstituted arylene group having 6 to 30 ring-forming carbon atoms or a substituted or unsubstituted heteroarylene group having 2 to 30 ring-forming carbon atoms.

In Formula E-2a, A₁ to A₅ may each independently be N or C(Rᵢ). In Formula E-2a, Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. For example, Rₐ to Rᵢ may be bonded to an adjacent group to form a hydrocarbon ring or a heterocycle containing N, O, S, etc., as a ring-forming atom.

In Formula E-2a, two or three of A₁ to A₅ may each be N, and the remainder of A₁ to A₅ may each independently be C(Rᵢ).

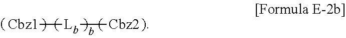

In Formula E-2b, Cbz1 and Cbz2 may each independently be an unsubstituted carbazole group or a carbazole group substituted with an aryl group having 6 to 30 ring-forming carbon atoms. In Formula E-2b, L_{b} may be a direct linkage, a substituted or unsubstituted arylene group having 6 to 30 ring-forming carbon atoms, or a substituted or unsubstituted heteroarylene group having 2 to 30 ring-forming carbon atoms. In Formula E-2b, b may be an integer from 0 to 10. When b is 2 or more, multiple L_{b} groups may each independently be a substituted or unsubstituted arylene group having 6 to 30 ring-forming carbon atoms or a substituted or unsubstituted heteroarylene group having 2 to 30 ring-forming carbon atoms.

In an embodiment, the compound represented by Formula E-2a or Formula E-2b may be any compound selected from Compound Group E-2. However, the compounds listed in Compound Group E-2 are only examples, and the compound represented by Formula E-2a or Formula E-2b is not limited to Compound Group E-2:

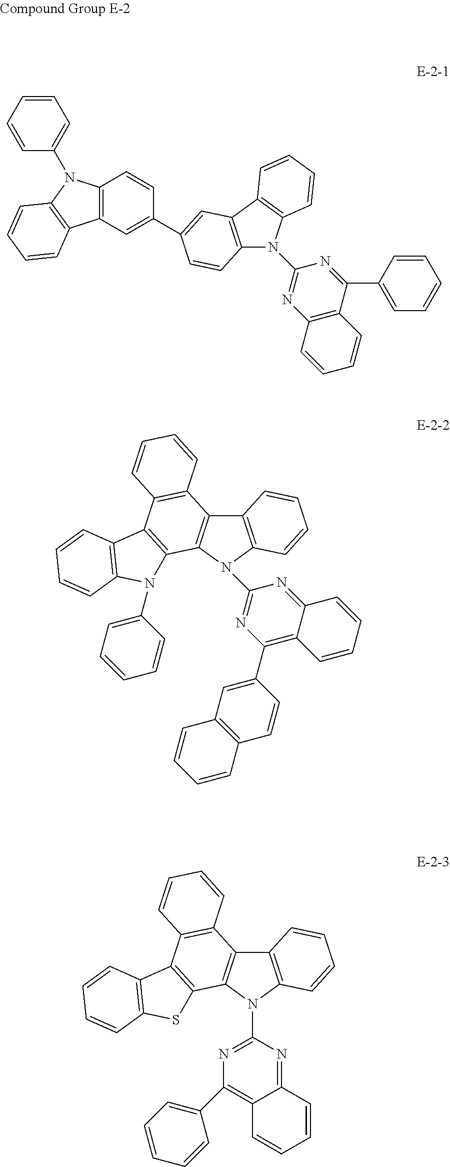 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

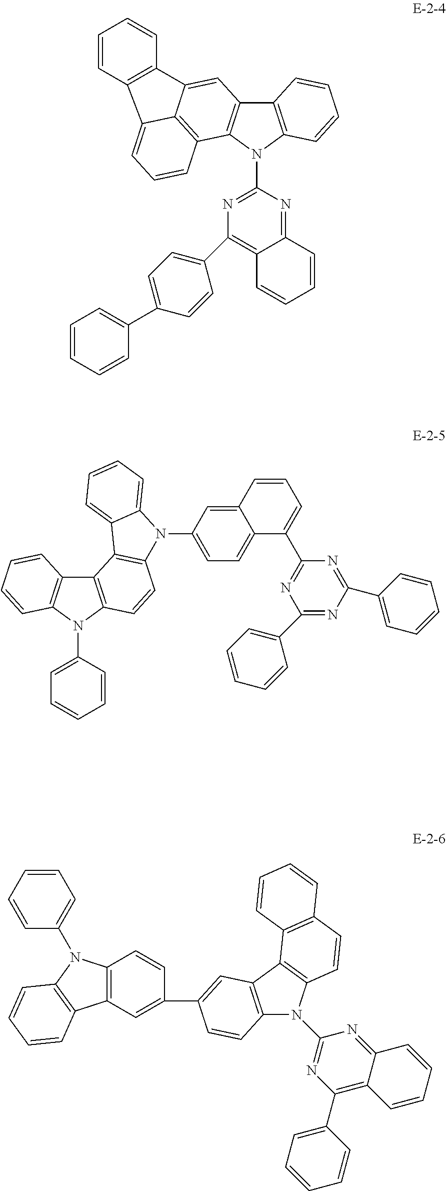 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

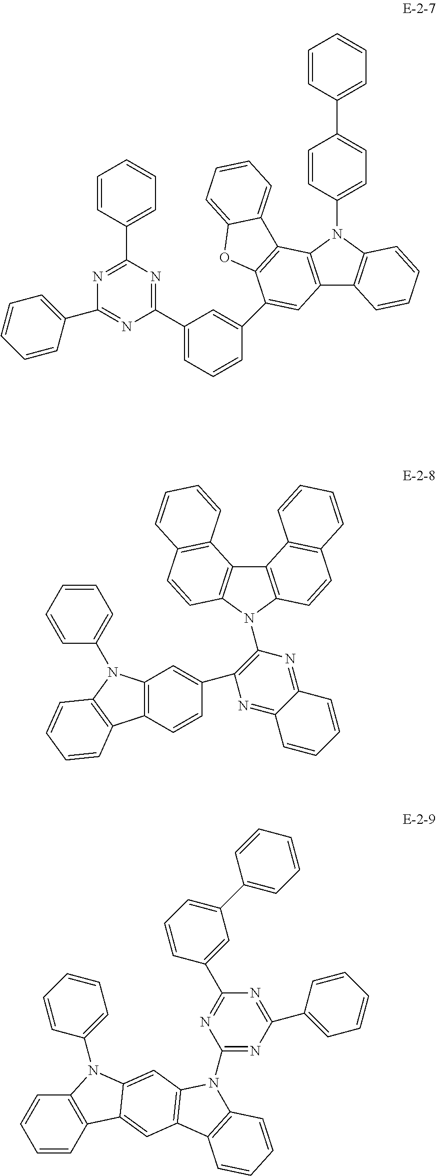 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

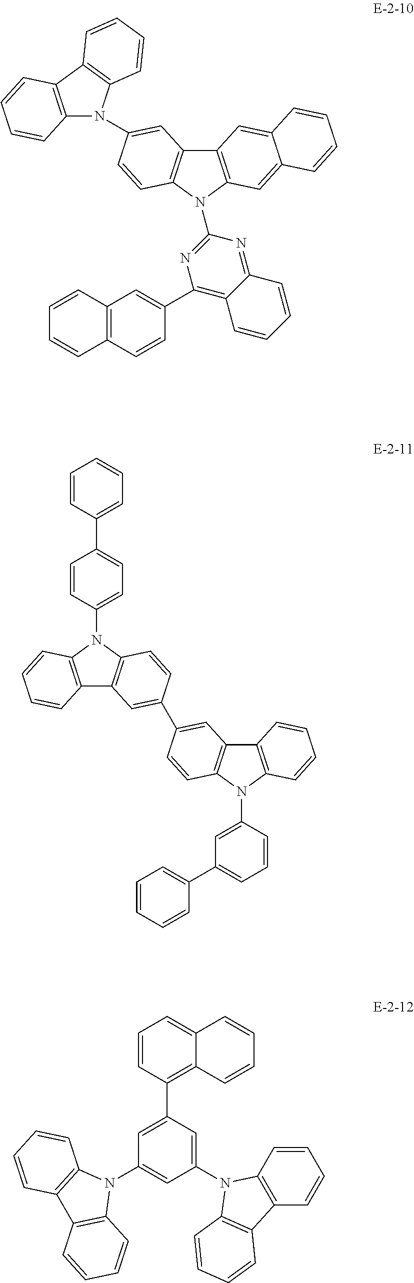 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

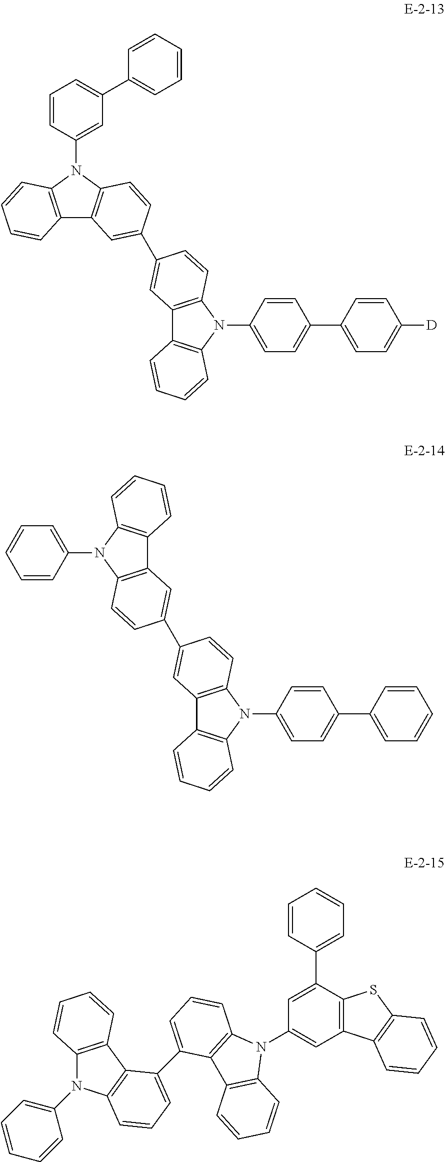 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

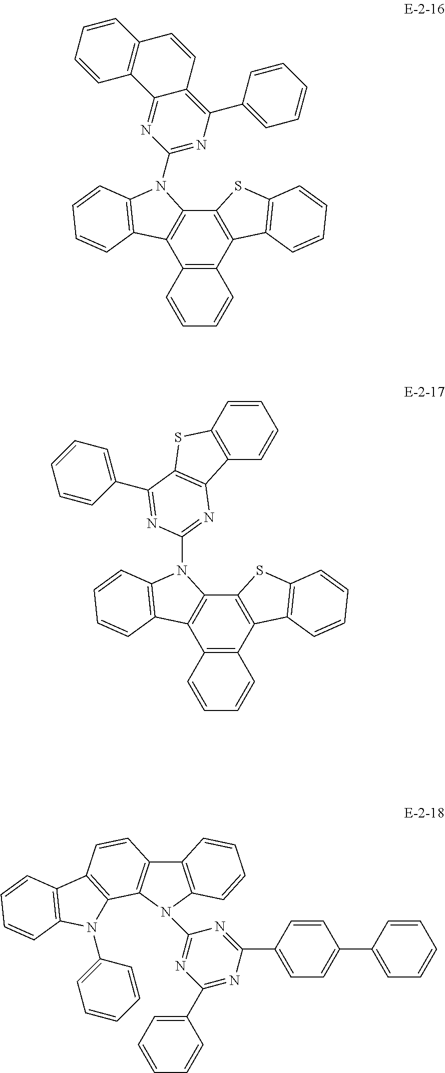 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

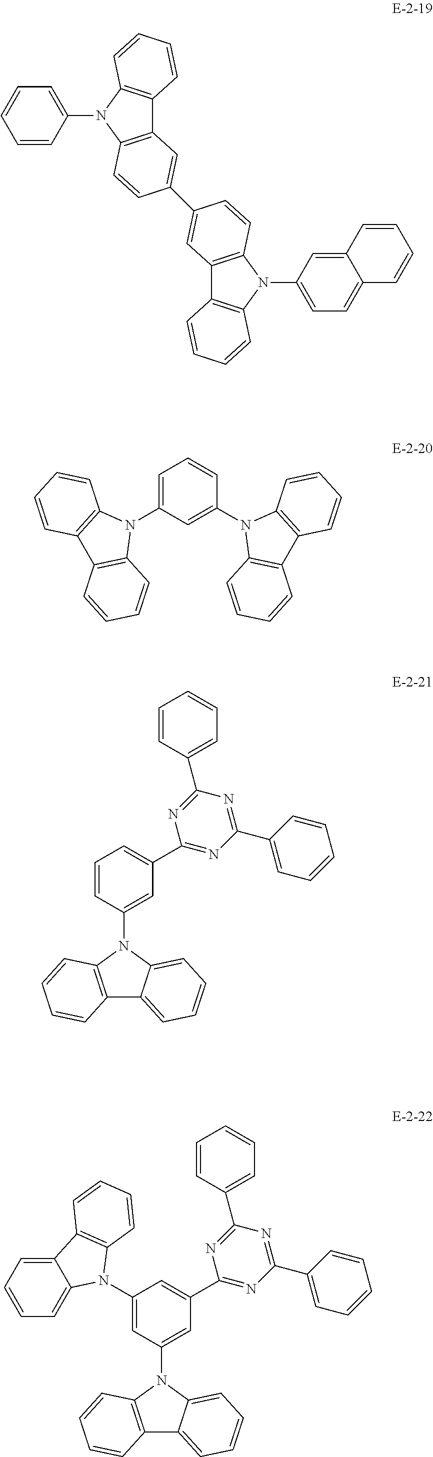 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

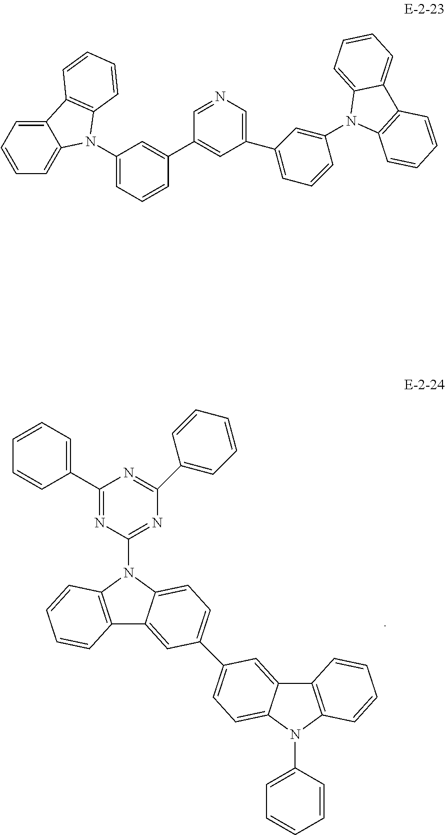 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a material of the related art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), and 1,3,5-tris(1-phenyl-1H-benzo[d]imidazole-2-yl)benzene (TPBi). However, embodiments are not limited thereto. For example, tris(8-hydroxyquinolino)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), etc. may be used as a host material.

In an embodiment, the emission layer EML may include a compound represented by Formula M-a or Formula M-b. The compound represented by Formula M-a or Formula M-b may be used as a phosphorescent dopant material:

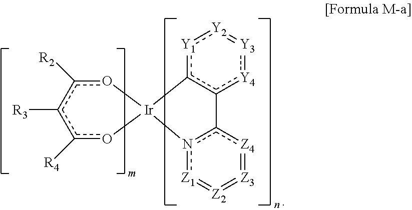

In Formula M-a, Y₁ to Y₄ and Z₁ to Z₄ may each independently be C(R₁) or N; and R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. In Formula M-a, m may be 0 or 1, and n may be 2 or 3. In Formula M-a, when m is 0, n may be 3, and when m is 1, n may be 2.

In an embodiment, the compound represented by Formula M-a may be any compound selected from Compound M-a1 to Compound M-a25. However, Compounds M-a1 to M-a25 are only examples, and the compound represented by Formula M-a is not limited to Compounds M-a1 to M-a25:

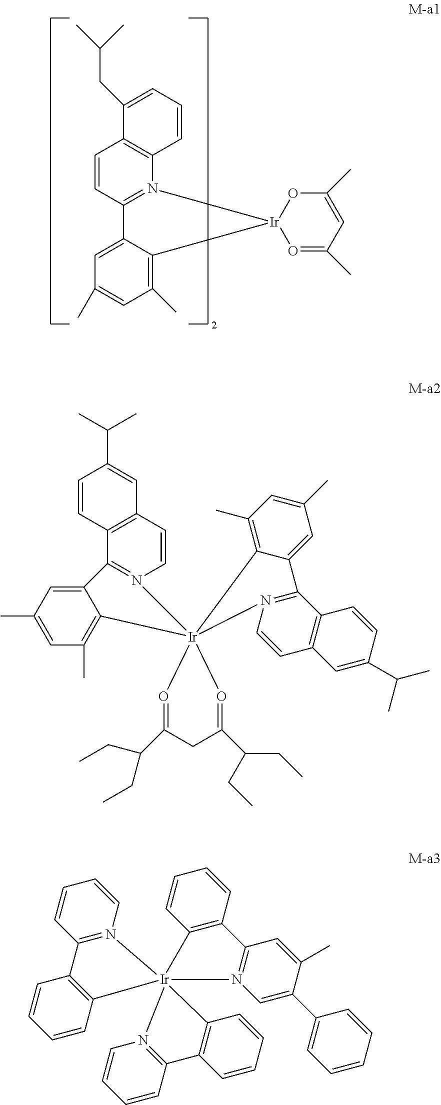

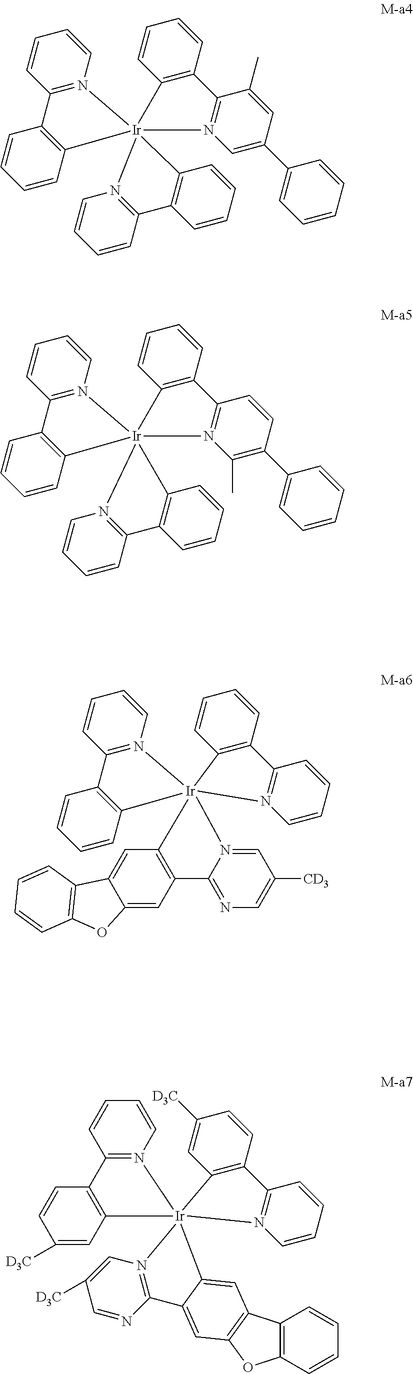

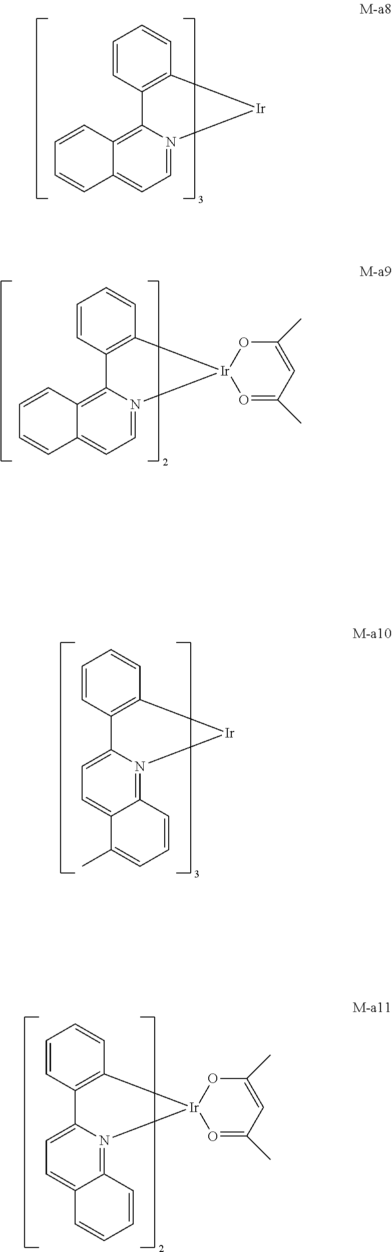

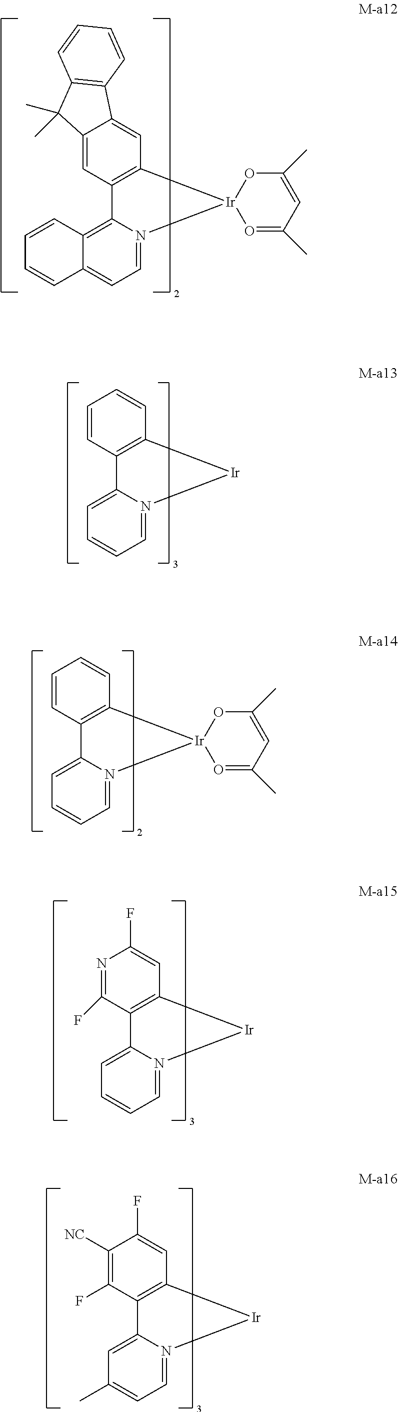

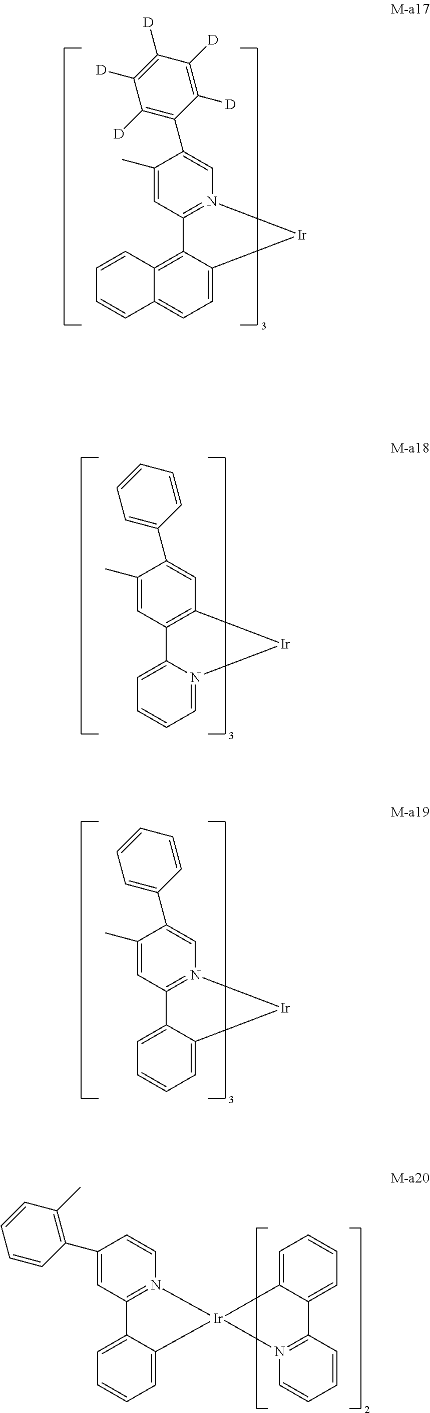

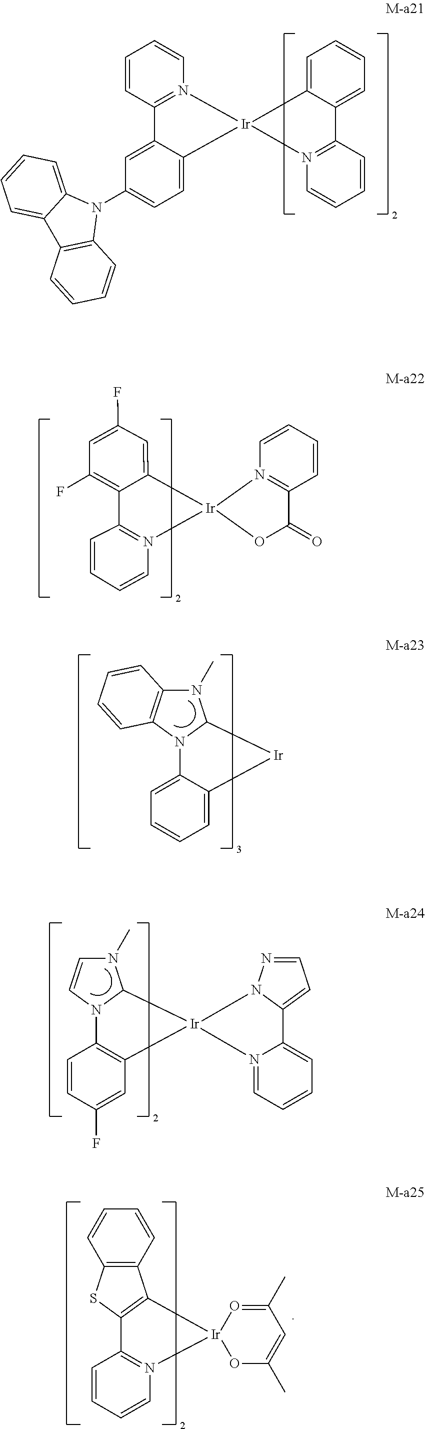

In embodiments, Compound M-a1 and Compound M-a2 may each be used as a red dopant material, and Compounds M-a3 to M-a5 may each be used as a green dopant material.

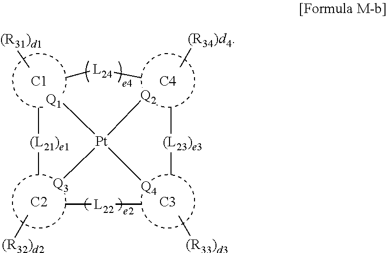

In Formula M-b, Q₁ to Q₄ may each independently be C or N. In Formula M-b, C₁ to C₄ may each independently be a substituted or unsubstituted hydrocarbon ring having 5 to 30 ring-forming carbon atoms, a substituted or unsubstituted aryl group having 6 to 30 ring-forming carbon atoms, or a substituted or unsubstituted heterocyclic group having 2 to 30 ring-forming carbon atoms.

In Formula M-b, L₂₁ to L₂₄ may each independently be a direct linkage,

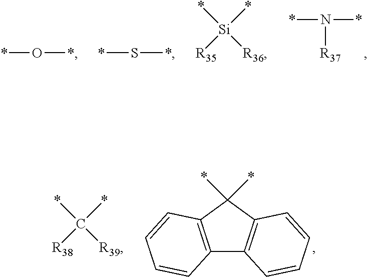

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms. In Formula M-b, el to e4 may each independently be 0 or 1.

In Formula M-b, R₃₁ to R₃₉ may each independently be a hydrogen atom, a deuterium atom, a halogen atom, a cyano group, a substituted or unsubstituted amine group, a substituted or unsubstituted alkyl group having 1 t to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. In Formula M-b, d1 to d4 may each independently be an integer from 0 to 4. If d1 is 2 or more, multiple R₃₁ groups may be the same as each other, or at least one thereof may be different from the remainder. If d2 is or more, multiple R₃₂ groups may be the same as each other, or at least one thereof may be different from the remainder. If d3 is 2 or more, multiple R₃₃ groups may be the same as each other, or at least one thereof may be different from the remainder. If d4 is 2 or more, multiple R₃₄ groups may be the same as each other, or at least one thereof may be different from the remainder.

A compound represented by Formula M-b may be used as a blue phosphorescent dopant or as a green phosphorescent dopant. In an embodiment, the compound represented by Formula M-b may be any compound selected from Compounds AD-01 to AD-40. However, Compounds AD-01 to AD-40 are only examples, and the compound represented by Formula M-b is not limited to Compounds AD-01 to AD-40. In Compounds AD-01 to AD-40, D represents a deuterium atom.

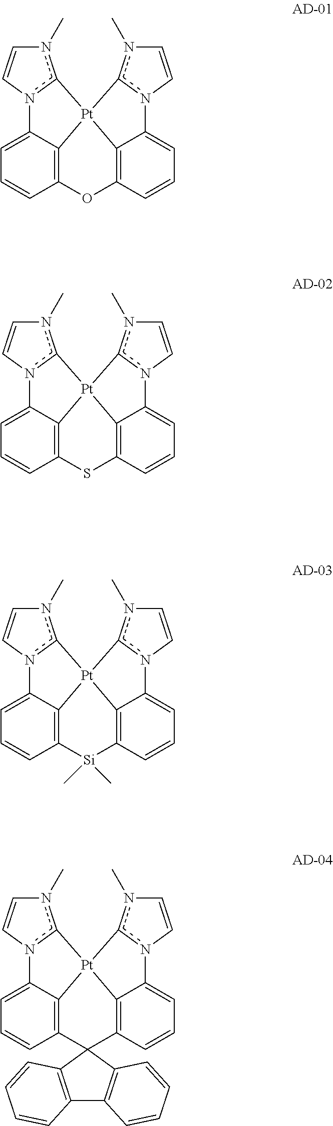

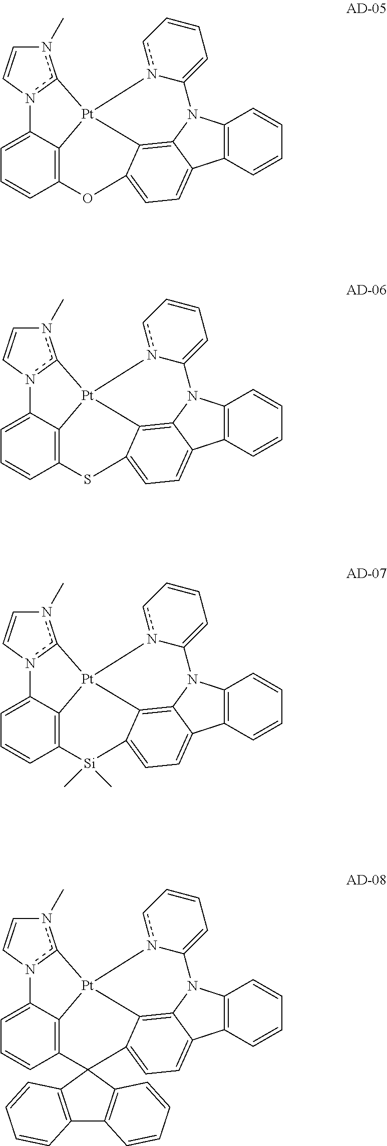

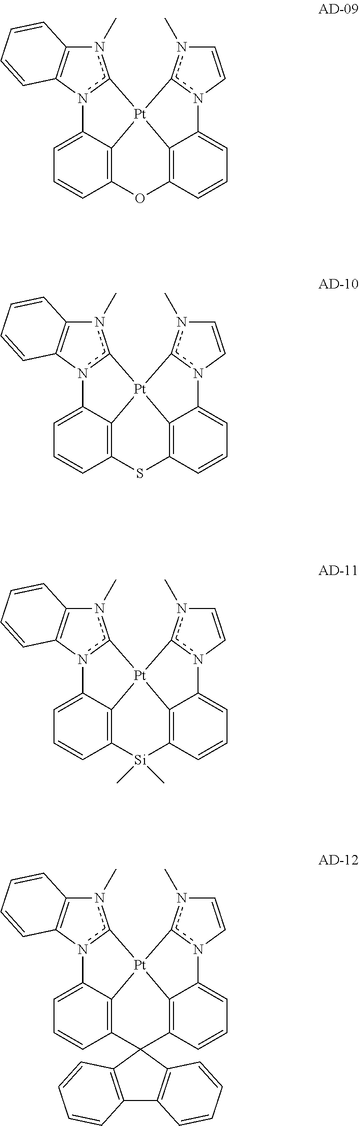

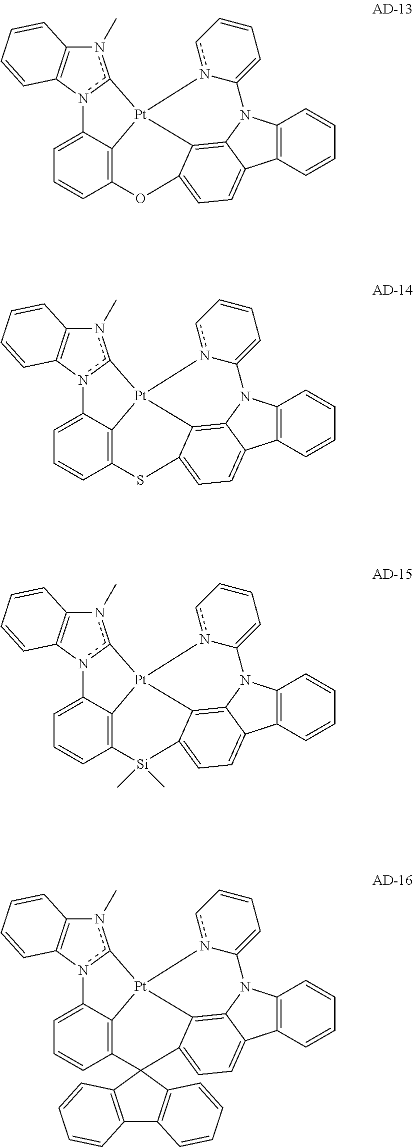

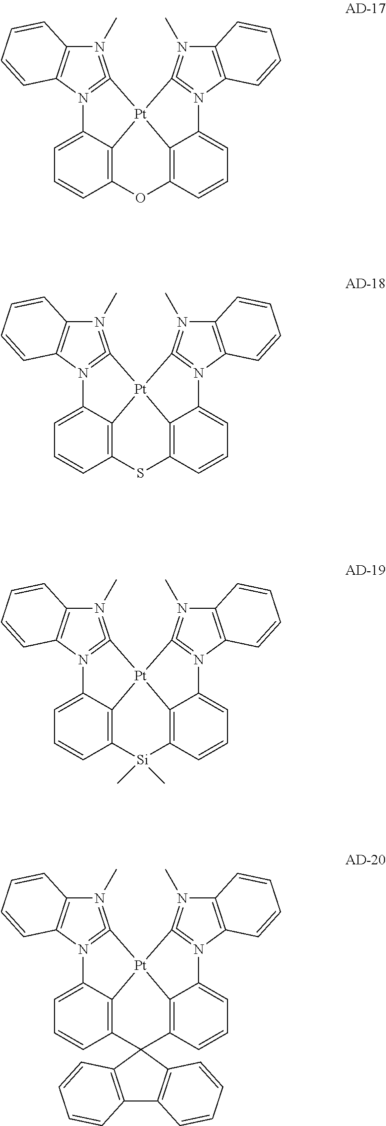

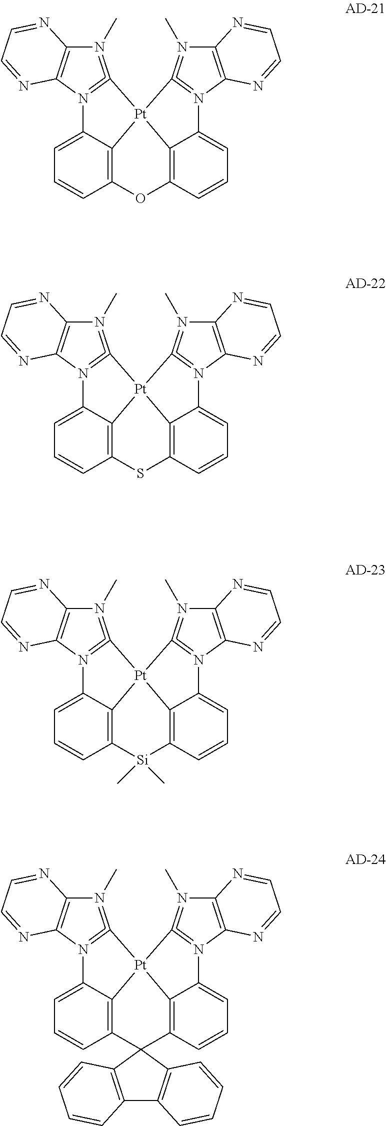

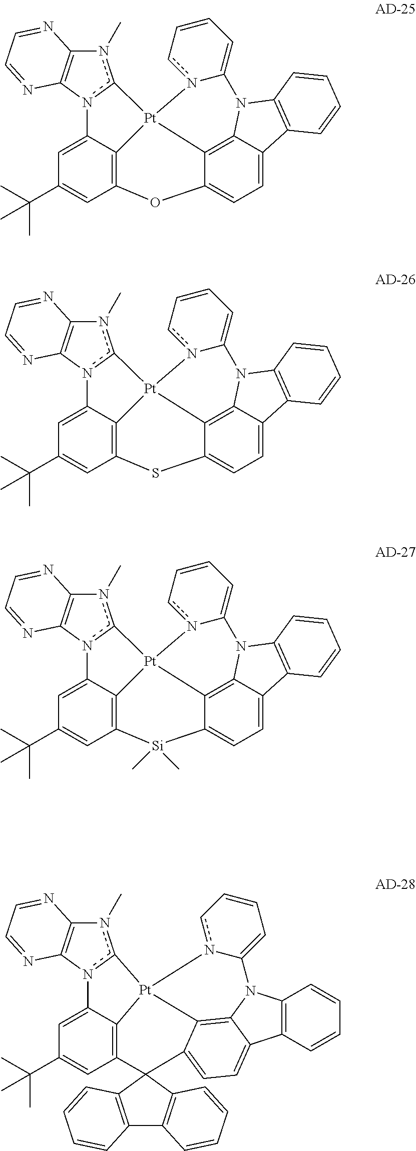

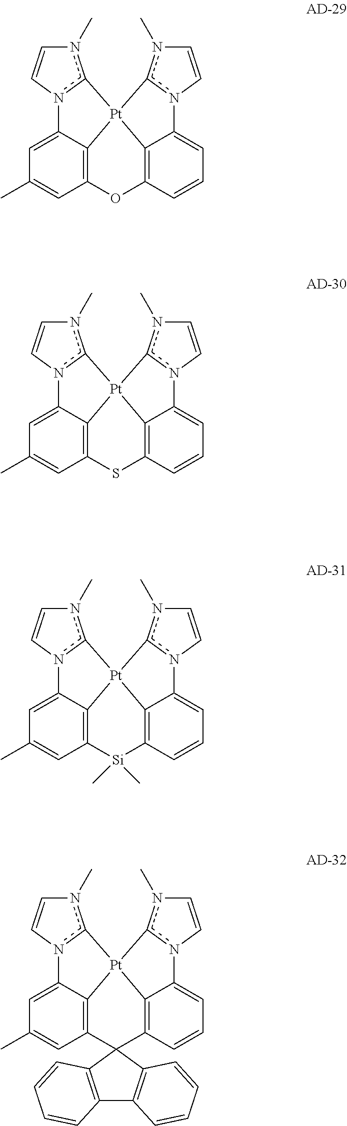

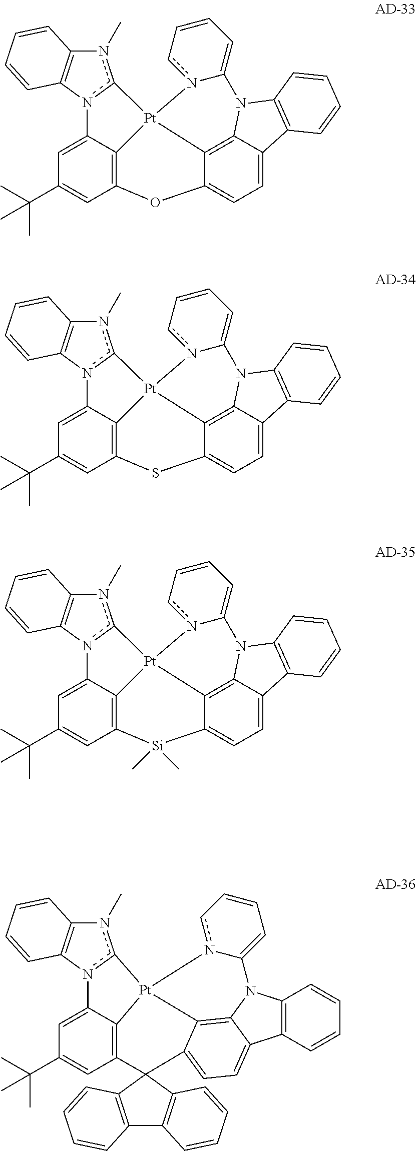

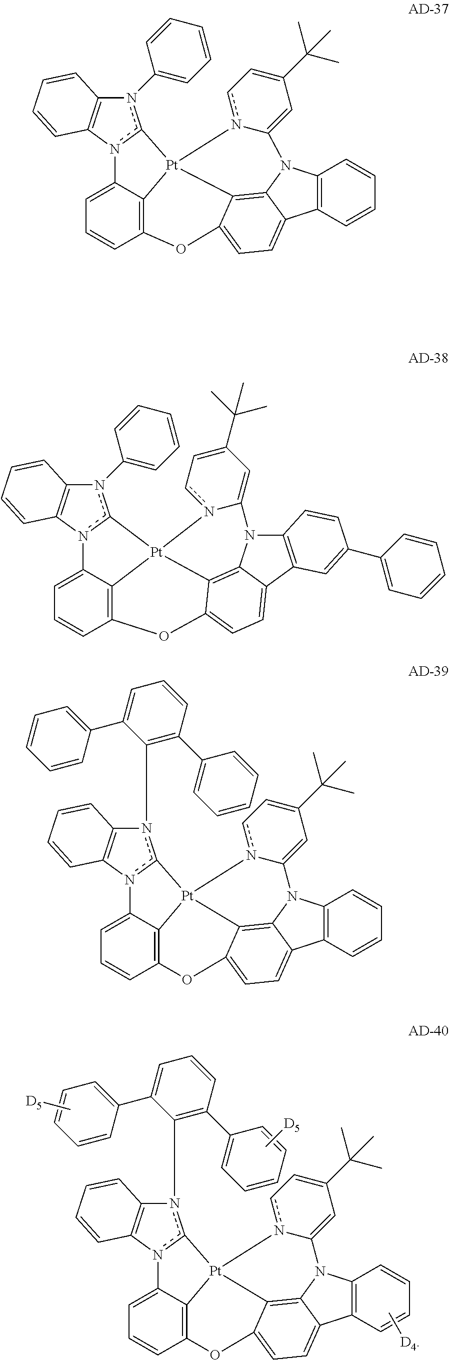

In an embodiment, the emission layer EML may include a compound represented by one of Formula F-a to Formula F-c. The compound represented by one of Formula F-a to Formula F-c may be used as a fluorescence dopant material.

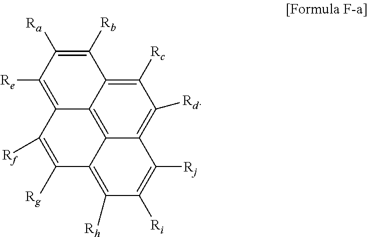

In Formula F-a, two of Rₐ to Rⱼ may each independently be substituted with a group represented by *—N—Ar₁Ar₂. The remainder of Rₐ to Rⱼ that are not substituted with the group represented by *—NAr₁Ar₂, may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In the group represented by *—NAr₁Ar₂, Ar₁ and Ar₂ may each independently be a substituted or unsubstituted aryl group having 6 to 30 ring-forming carbon atoms or a substituted or unsubstituted heteroaryl group having 2 to 30 ring-forming carbon atoms. For example, at least one of Ar₁ and Ar₂ may each independently be a heteroaryl group containing O or S as a ring-forming atom.

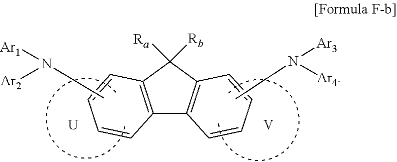

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring. In Formula F-b, Ar₁ to Ar₄ may each independently be a substituted or unsubstituted aryl group having 6 to 30 ring-forming carbon atoms or a substituted or unsubstituted heteroaryl group having 2 to 30 ring-forming carbon atoms. For example, at least one of Ar₁ to Ar₄ may each independently be a heteroaryl group containing O or S as a ring-forming atom.

In Formula F-b, U and V may each independently be a substituted or unsubstituted hydrocarbon ring having 5 to 30 ring-forming carbon atoms or a substituted or unsubstituted heterocycle having 2 to 30 ring-forming carbon atoms.

In Formula F-b, the number of rings represented by U and V may each independently be 0 or 1. In Formula F-b, when the number of U or V is 1, a fused ring may be present at a portion indicated by U or V, and when the number of U or V is 0, a fused ring may not be present at the portion indicated by U or V. When the number of U is 0 and the number of V is 1, or when the number of U is 1 and the number of V is 0, a fused ring having a fluorene core of Formula F-b may be a cyclic compound having four rings. When the number of U and V is each 0, a fused ring having a fluorene core of Formula F-b may be a cyclic compound having three rings. When the number of U and V is each 1, a fused ring having a fluorene core of Formula F-b may be a cyclic compound having five rings.

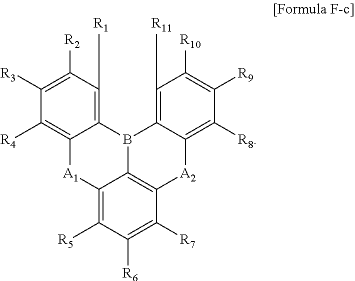

In Formula F-c, A₁ and A₂ may each independently be O, S, Se, or N(Rₘ); and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula F-c, R₁ to R₁₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or bonded to an adjacent group to form a ring.

In Formula F-c, A₁ and A₂ may each independently be bonded to a substituent of an adjacent ring to form a fused ring. For example, when A₁ and A₂ are each independently N(Rₘ), A₁ may be bonded to R₄ or R₅ to form a fused ring. For example, A₂ may be bonded to R₇ or R₈ to form a fused ring.

In an embodiment, the emission layer EML may further include, as a dopant material of the related art, a styryl derivative (e.g., 1,4-bis [2-(3-N-ethylcarbazolyl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N′-diphenylamino)phenyl)vinyl]biphenyl (DPAVBi)), perylene or a derivative thereof (e.g., 2,5,8,11-tetra-t-butylperylene (TBP)), pyrene or a derivative thereof (e.g., 1,1′-dipyrene, 1,4-dipyrenylbenzene, and 1,4-bis(N,N′-diphenylamino)pyrene), etc.

The emission layer EML may further include a phosphorescence dopant material of the related art. For example, a metal complex containing iridium (Ir), platinum (Pt), osmium (Os), gold (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2′)picolinate (FIrpic), bis(2,4-difluorophenylpyridinato)-tetrakis(1-pyrazolyl)borate iridium(III) (Fir6), or platinum octaethyl porphyrin (PtOEP) may be used as a phosphorescent dopant. However, embodiments are not limited thereto.

In an embodiment, the emission layer EML may include a quantum dot material. The quantum dot may be a Group II-VI compound, a Group I-II-VI compound, a Group II-IV-VI compound, a Group I-II-IV-VI compound, a Group II-IV-V compound, a Group III-VI compound, a Group I-III-VI compound, a Group III-V compound, a Group III-II-V compound, a Group IV-VI compound, a Group IV element, a Group IV compound, or any combination thereof.

Examples of a Group II-VI compound may include: binary compounds such as CdSe, CdTe, CdS, ZnS, ZnSe, ZnTe, ZnO, HgS, HgSe, HgTe, MgSe, MgS, and a mixture thereof; ternary compounds such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof; and quaternary compounds such as HgZnTeS, CdZnSeS, CdZnSeTe, CdZnSTe, CdHgSeS, CdHgSeTe, CdHgSTe, HgZnSeS, HgZnSeTe, and a mixture thereof.

In an embodiment, a Group II-VI compound may further include a Group I metal and/or a Group IV element. Examples of a Group I-II-VI compound may include CuSnS and CuZnS.

Examples of a Group II-IV-VI compound may include ZnSnS or the like. Examples of a Group I-II-IV-VI compound may include quaternary compounds such as Cu₂ZnSnS₂, Cu₂ZnSnS₄, Cu₂ZnSnSe₄, Ag₂ZnSnS₂, and a mixture thereof.

Examples of a Group II-IV-V compound may include ternary compounds such as ZnSnP, ZnSnP₂, ZnSnAs₂, ZnGeP₂, ZnGeAs₂, CdSnP₂, CdGeP₂, and a mixture thereof.

Examples of a Group III-VI compound may include: binary compounds such as GaS, Ga₂S₃, GaSe, Ga₂Se₃, GaTe, InTe, InS, InSe, In₂S₃, and In₂Se₃; ternary compounds such as InGaS₃, and InGaSe₃; and any combination thereof.

Examples of a Group I-III-VI compound may include: ternary compounds such as AgInS₂, CuInS, CuInS₂, AgGaS₂, CuGaS₂ CuGaO₂, AgGaO₂, AgAlO₂, and a mixture thereof; and quaternary compounds such as AgInGaS₂ and CuInGaS₂.

Examples of a Group III-V compound may include: binary compounds such as GaN, GaP, GaAs, GaSb, AlN, AlP, AlAs, AlSb, InN, InP, InAs, InSb, and a mixture thereof; ternary compounds such as GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and a mixture thereof; and quaternary compounds such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In an embodiment, a Group III-V compound may further include a Group II metal. Examples of a Group III-II-V compound may include InZnP or the like.

Examples of a Group IV-VI compound may include: binary compounds such as SnS, SnSe, SnTe, PbS, PbSe, PbTe, and a mixture thereof; ternary compounds such as SnSeS, SnSeTe, SnSTe, PbSeS, PbSeTe, PbSTe, SnPbS, SnPbSe, SnPbTe, and a mixture thereof; and quaternary compounds such as SnPbSSe, SnPbSeTe, SnPbSTe, and a mixture thereof.

Examples of a Group IV element may include Si, Ge, and a mixture thereof. Examples of a Group IV compound may include a binary compound such as SiC, SiGe, and a mixture thereof.

Each element included in a compound, such as a binary compound, a ternary compound, or a quaternary compound, may be present in a particle at a uniform concentration distribution or at a non-uniform concentration distribution. For example, a formula may indicate the elements that are included in a compound, and an elemental ratio of the compound may vary.

For example, AgInGaS₂ may indicate AgInₓGa₁₋ₓS₂ (where x is a real number between 0 to 1).

In an embodiment, a quantum dot may have a single structure or a quantum dot may have a core-shell structure. For example, a material included in the core may be different from a material included in the shell.

The shell of a quantum dot may serve as a protection layer that prevent chemical deformation of the core to maintain semiconductor properties, and/or may serve as a charging layer that imparts electrophoretic properties to the quantum dot. The shell may be single-layered or multilayered. An interface between the core and the shell may have a concentration gradient in which the concentration of an element that is present in the shell decreases towards the core.

In embodiments, the quantum dot may have a core-shell structure that includes a core and a shell surrounding the core. Examples of a shell of a quantum dot may include a metal oxide, a non-metal oxide, a semiconductor compound, and a combination thereof.

Examples of a metal oxide or a non-metal oxide may include binary compounds such as SiO₂, Al₂O₃, TiO₂, ZnO, MnO, Mn₂O₃, Mn₃O₄, CuO, FeO, Fe₂O₃, Fe₃O₄, CoO, Co₃O₄, and NiO; and ternary compounds such as MgAl₂O₄, CoFe₂O₄, NiFe₂O₄, and CoMn₂O₄, but embodiments are not limited thereto.

Examples of a semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, and the like, but embodiments are not limited thereto.

The quantum dot may have a full width at half maximum (FWHM) of an emission spectrum equal to or less than about 45 nm. For example, the quantum dot may have a FWHM of an emission spectrum equal to or less than about 40 nm. For example, the quantum dot may have a FWHM of an emission spectrum equal to or less than about 30 nm. Within any of the above ranges, color purity or color reproducibility may be improved. Light emitted through a quantum dot may be emitted in all directions, so that a wide viewing angle may be improved.

The form of a quantum dot is not particularly limited, and may be any form used in the related art. For example, a quantum dot may have a spherical shape, a pyramidal shape, a multi-arm shape, or a cubic shape, or a quantum dot may be in the form of nanoparticles, nanotubes, nanowires, nanofibers, nanoplate particles, etc.

As a size of a quantum dot is adjusted or an elemental ratio of a quantum dot compound is adjusted, an energy band gap may be adjusted accordingly, so that light in various wavelength ranges may be obtained from a quantum dot emission layer. Therefore, by utilizing quantum dots as described above (for example, by using different sizes of quantum dots or different elemental ratios in a quantum dot compound), a light-emitting element that emits light in various wavelengths may be implemented. For example, a size of a quantum dot or an elemental ratio of a quantum dot compound may be adjusted to emit red light, green light, and/or blue light. In an embodiment, quantum dots may be configured to emit white light by combining various colors of light.

In the light-emitting elements ED according to embodiments, as shown in each of FIGS. 3 to 6, the electron transport region ETR may be provided on the emission layer EML. The electron transport region ETR may include at least one of a hole blocking layer HBL, an electron transport layer ETL, and an electron injection layer EIL, but embodiments are not limited thereto.

The electron transport region ETR may have a structure consisting of a layer consisting of a single material, a structure consisting of a layer including different materials, or a structure including multiple layers including different materials.

For example, the electron transport region ETR may have a single layer structure of an electron injection layer EIL or an electron transport layer ETL, or may have a single layer structure formed of an electron injection material and an electron transport material. In embodiments, the electron transport region ETR may have a single layer structure formed of different materials, or may have a structure in which an electron transport layer ETL/electron injection layer EIL, or a hole blocking layer HBL/electron transport layer ETL/electron injection layer EIL are stacked in its respective stated order from the emission layer EML, but embodiments are not limited thereto. The electron transport region ETR may have a thickness, for example, in a range of about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

In the light-emitting element ED according to an embodiment, the electron transport region ETR may include a compound represented by Formula ET-2:

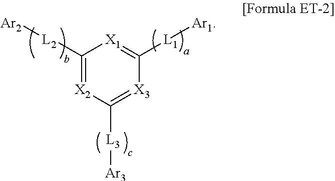

In Formula ET-2, at least one of X₁ to X₃ may each be N; and the remainder of X₁ to X₃ may each independently be C(Rₐ). In Formula ET-2, Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. In Formula ET-2, Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer from 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. When a to c are each 2 or more, multiple groups of each of L₁ to L₃ may each independently be a substituted or unsubstituted arylene group having 6 to 30 ring-forming carbon atoms or a substituted or unsubstituted heteroarylene group having 2 to 30 ring-forming carbon atoms.

The electron transport region ETR may include an anthracene-based compound. However, embodiments are not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,08)-(1,1′-biphenyl-4-olato)aluminum (BAlq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), or a mixture thereof.

In an embodiment, the electron transport region ETR may include at least one compound selected from Compound ET1 to Compound ET36:

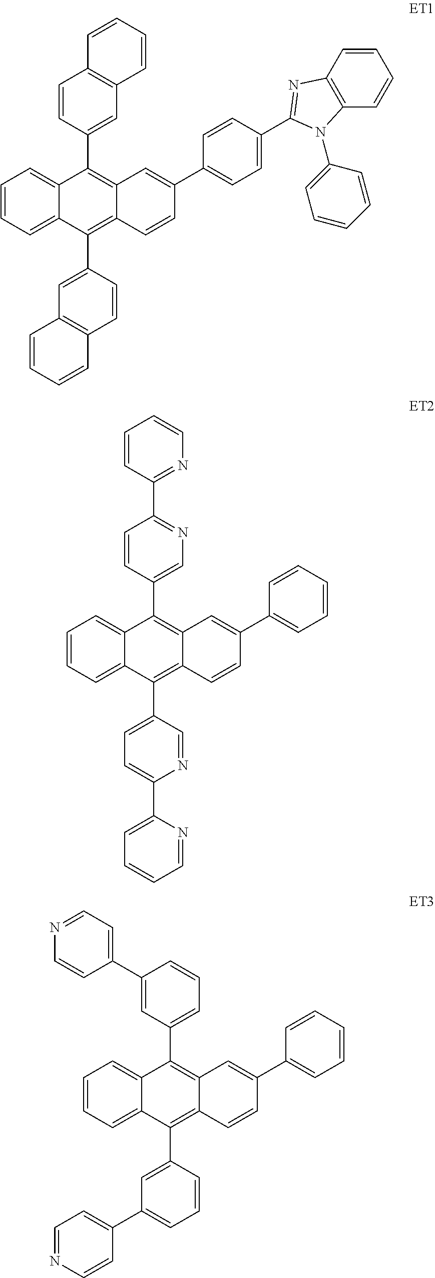 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

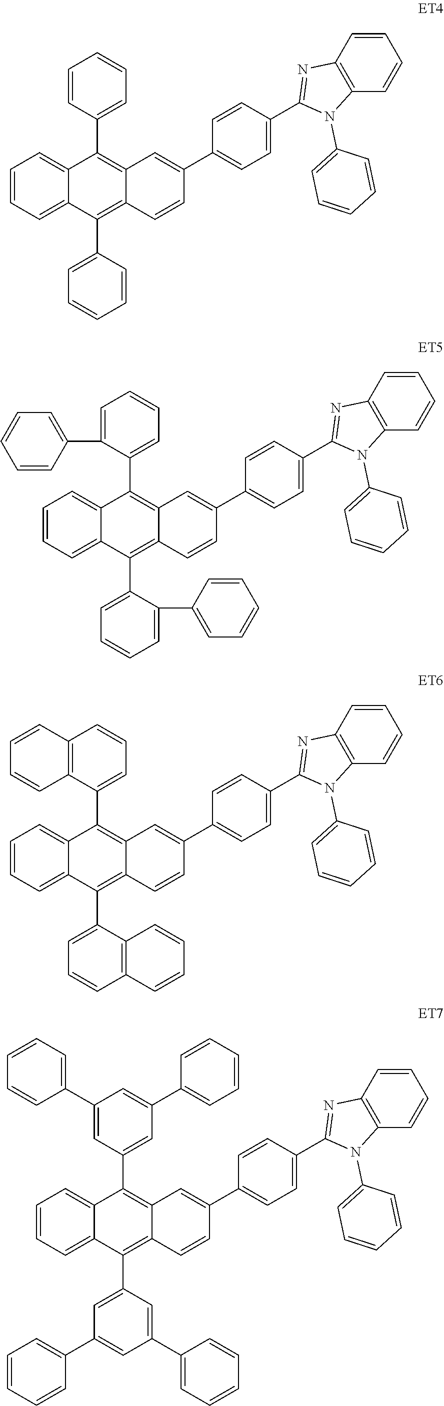 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

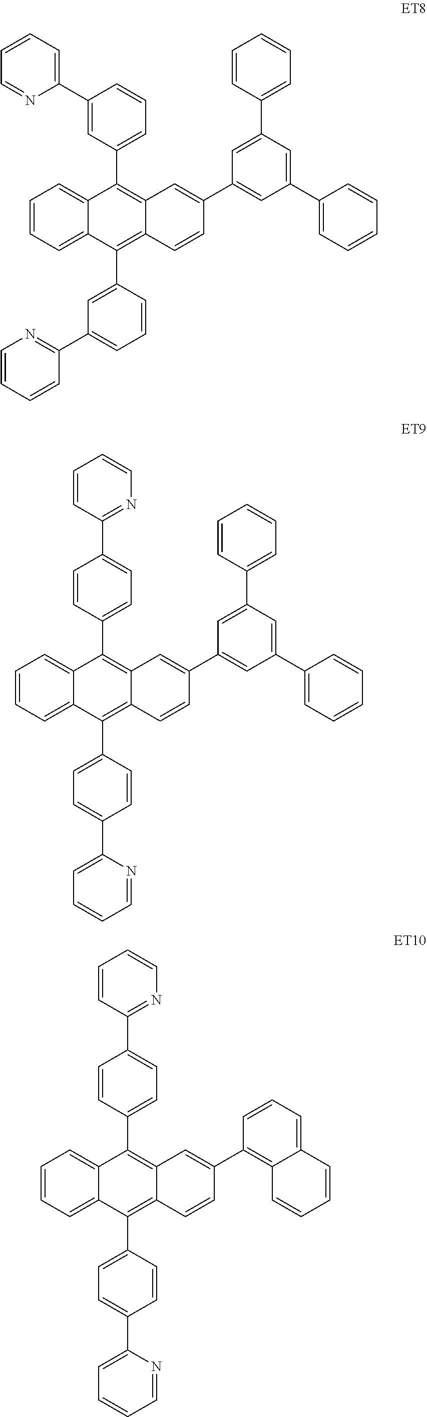

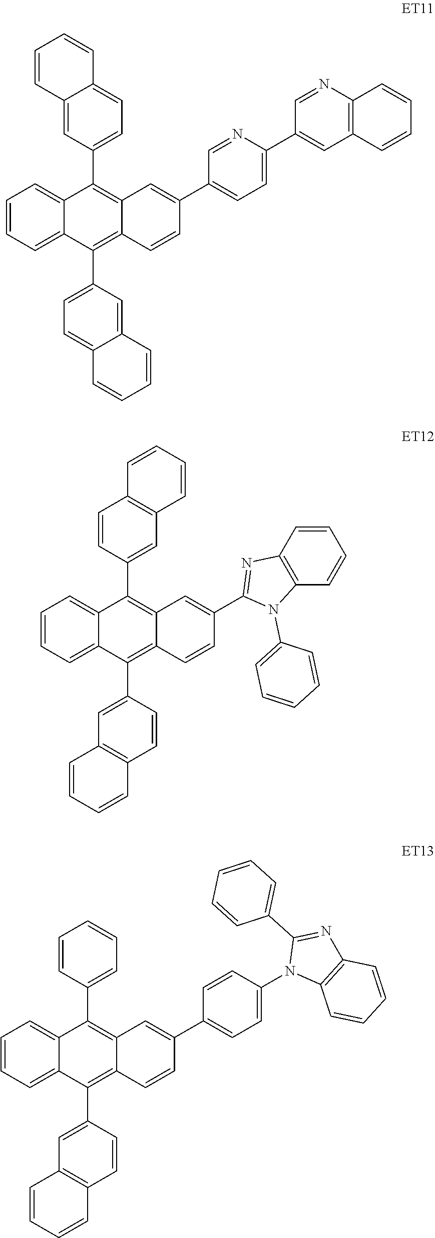 [SMILES: c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1 ; c1ccc(-c2c3ccccc3c(-c3ccc4ccccc4c3)c3ccc(-c4ccc(-n5c(-c6ccccc6)nc6ccccc65)cc4)cc23)cc1]

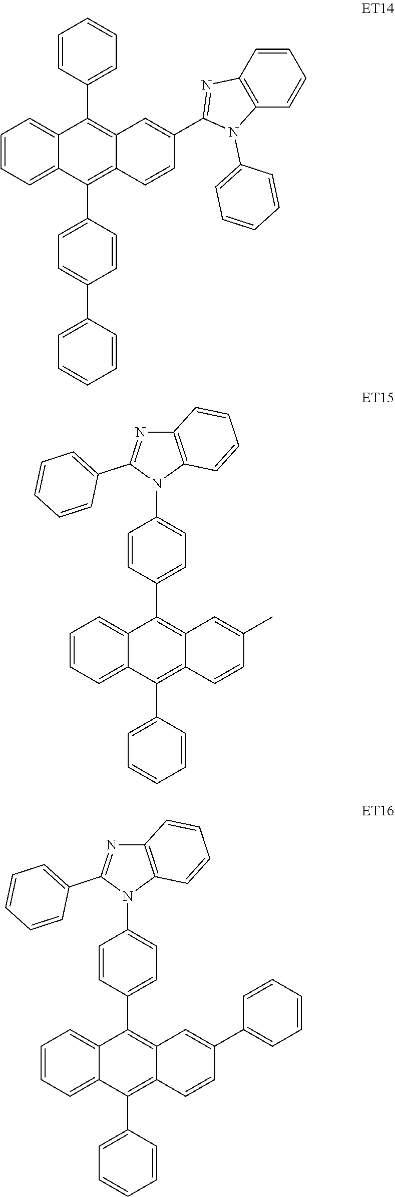

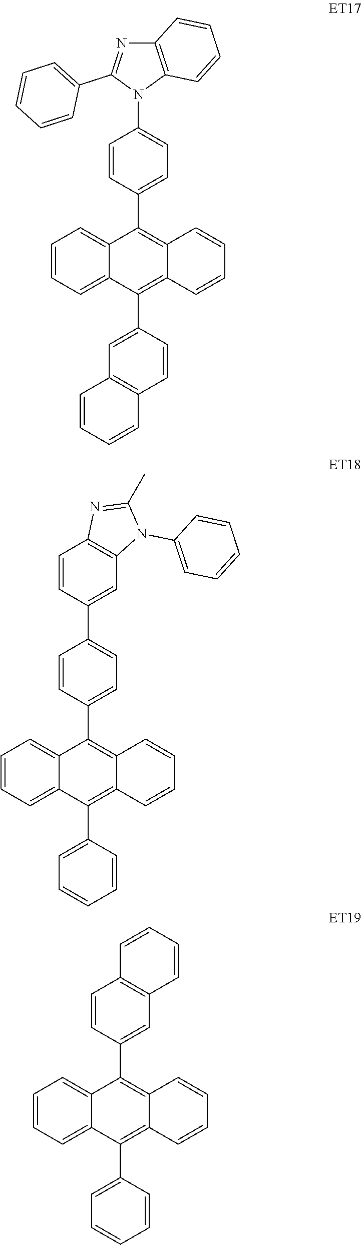

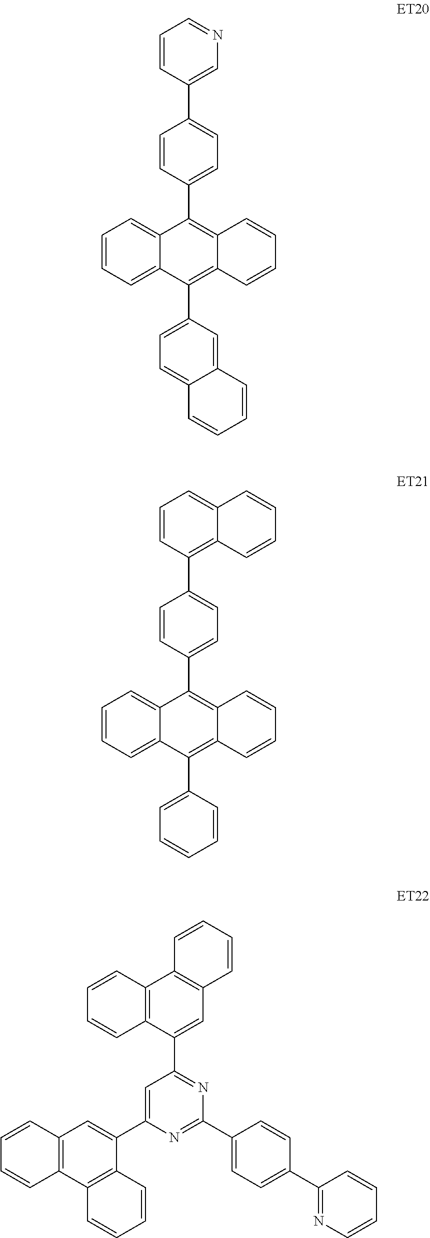

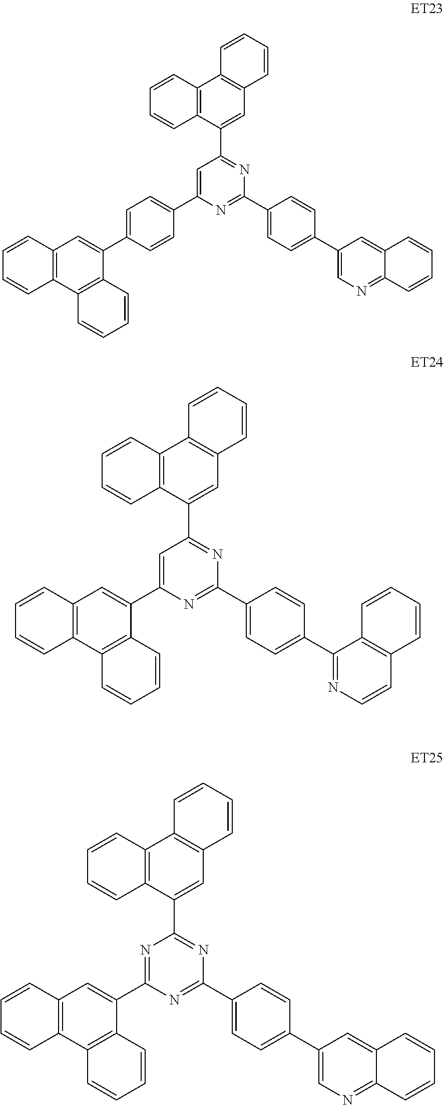 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

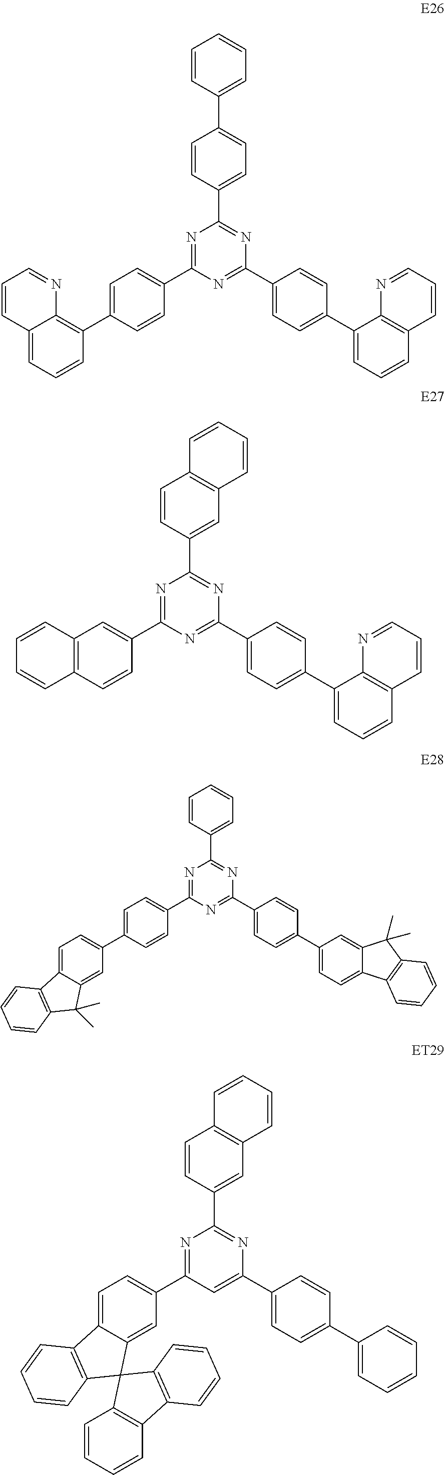 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1]

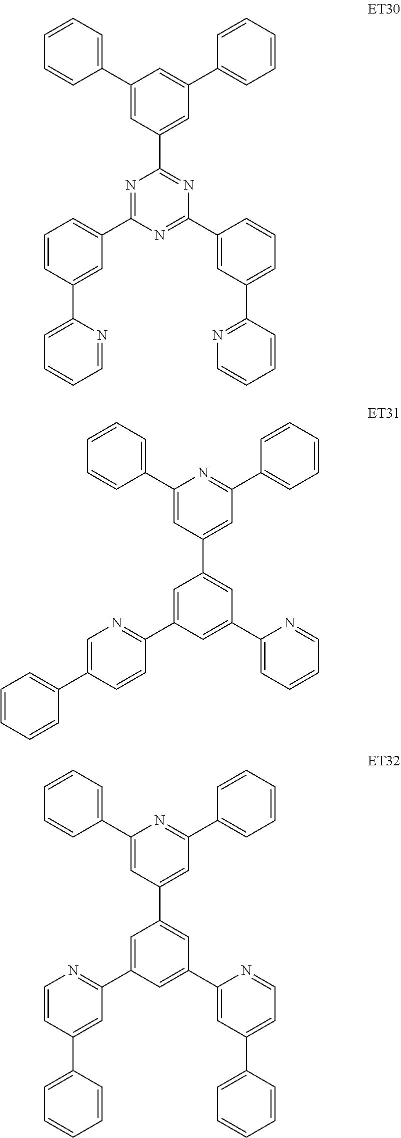 [SMILES: c1ccc(-c2cc(-c3ccccc3)cc(-c3nc(-c4cccc(-c5ccccn5)c4)nc(-c4cccc(-c5ccccn5)c4)n3)c2)cc1 ; c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1]

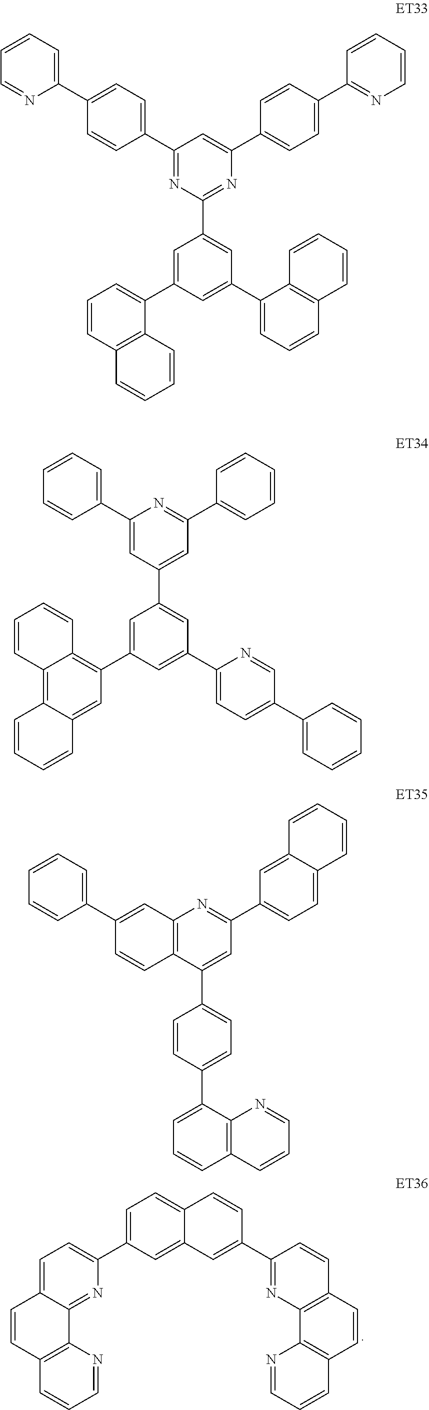

In an embodiment, the electron transport region ETR may include: a metal halide such as LiF, NaCl, CsF, RbCl, RbI, CuI, and KI; a lanthanide metal such as Yb; or a co-deposited material of a metal halide and a lanthanide metal. For example, the electron transport region ETR may include KI:Yb, RbI:Yb, LiF:Yb, etc., as a co-deposited material. The electron transport region ETR may include a metal oxide such as Li₂O or BaO, or 8-hydroxyl-lithium quinolate (Liq), etc., but embodiments are not limited thereto. The electron transport region ETR may also be formed of a mixture material of an electron transport material and an insulating organometallic salt. The organometallic salt may be a material having an energy band gap equal to or greater than about 4 eV. For example, the organometallic salt may include a metal acetate, a metal benzoate, a metal acetoacetate, a metal acetylacetonate, or a metal stearate.

The electron transport region ETR may further include at least one of 2,9-dimethyl-4,7-diphenyl-1,10-phenanthroline (BCP), diphenyl(4-(triphenylsilyl)phenyl)phosphine oxide (TSPO1), and 4,7-diphenyl-1,10-phenanthroline (Bphen), in addition to the above-described materials, but embodiments are not limited thereto.

The electron transport region ETR may include the above-described compounds of the electron transport region ETR in at least one of an electron injection layer EIL, an electron transport layer ETL, and a hole blocking layer HBL.

When the electron transport region ETR includes an electron transport layer ETL, the electron transport layer ETL may have a thickness in a range of about 100 Å to about 1,000 Å. For example, the electron transport layer ETL may have a thickness in a range of about 150 Å to about 500 Å. If the thickness of the electron transport layer ETL satisfies any of the aforementioned ranges, satisfactory electron transport characteristics may be obtained without a substantial increase in driving voltage. When the electron transport region ETR includes an electron injection layer EIL, the electron injection layer EIL may have a thickness in a range of about 1 Å to about 100 Å. For example, the electron injection layer EIL may have a thickness in a range of about 3 Å to about 90 Å. If the thickness of the electron injection layer EIL satisfies any of the above-described ranges, satisfactory electron injection characteristics may be obtained without a substantial increase in driving voltage.

The second electrode EL2 may be provided on the electron transport region ETR. The second electrode EL2 may be a common electrode. The second electrode EL2 may be a cathode or an anode, but embodiments are not limited thereto. For example, when the first electrode EL1 is an anode, the second electrode EL2 may be a cathode, and when the first electrode EL1 is a cathode, the second electrode EL2 may be an anode.

The second electrode EL2 may be a transmissive electrode, a transflective electrode, or a reflective electrode. When the second electrode EL2 is a transmissive electrode, the second electrode EL2 may be formed of a transparent metal oxide, for example, indium tin oxide (ITO), indium zinc oxide (IZO), zinc oxide (ZnO), indium tin zinc oxide (ITZO), etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, a compound thereof, or a mixture thereof (e.g., AgMg, AgYb, or MgYb). In an embodiment, the second electrode EL2 may have a multilayer structure including a reflective layer or a transflective layer formed of the above-described materials, and a transparent conductive layer formed of ITO, IZO, ZnO, ITZO, etc. For example, the second electrode EL2 may include the above-described metal materials, combinations of at least two of the above-described metal materials, oxides of the above-described metal materials, or the like.

Although not shown in the drawings, the second electrode EL2 may be electrically connected to an auxiliary electrode. If the second electrode EL2 is electrically connected to an auxiliary electrode, resistance of the second electrode EL2 may decrease.

In an embodiment, the light-emitting element ED may further include a capping layer CPL disposed on the second electrode EL2. The capping layer CPL may have a multilayered structure or a single-layered structure.

In an embodiment, the capping layer CPL may include an organic layer or an inorganic layer. For example, when the capping layer CPL includes an inorganic material, the inorganic material may include an alkaline metal compound (e.g., LiF), an alkaline earth metal compound (e.g., MgF₂), SiON, SiNₓ, SiO_{y}, etc.

For example, when the capping layer CPL includes an organic material, the organic material may include a-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), etc., or the organic material may include an epoxy resin, or an acrylate such as methacrylate. However, embodiments are not limited thereto. In an embodiment, the capping layer CPL may include at least one of Compounds P1 to P5:

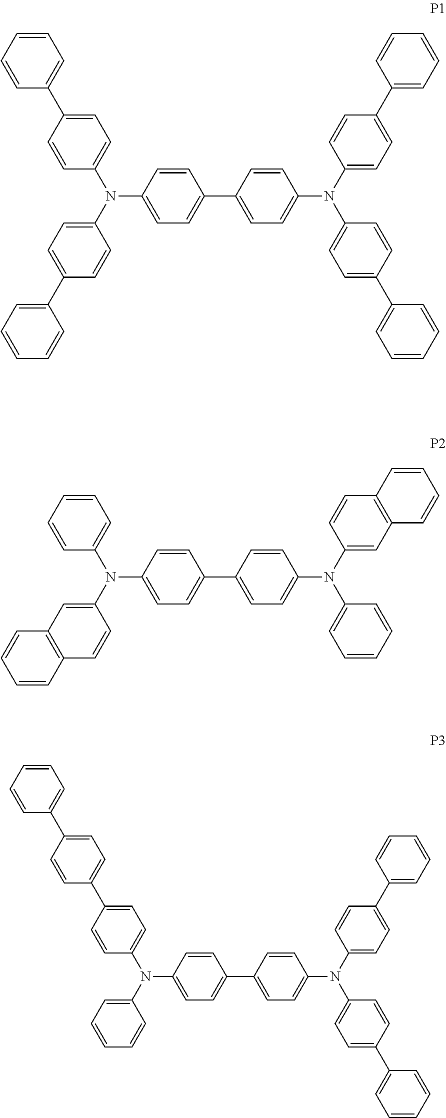 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

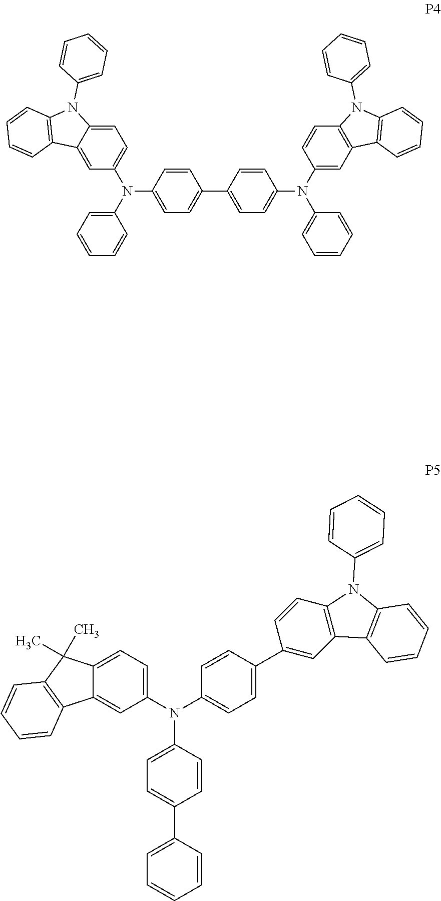 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

A refractive index of the capping layer CPL may be equal to or greater than about 1.6. For example, the refractive index of the capping layer CPL may be equal to or greater than about 1.6 with respect to light in a wavelength range of about 550 nm to about 660 nm.

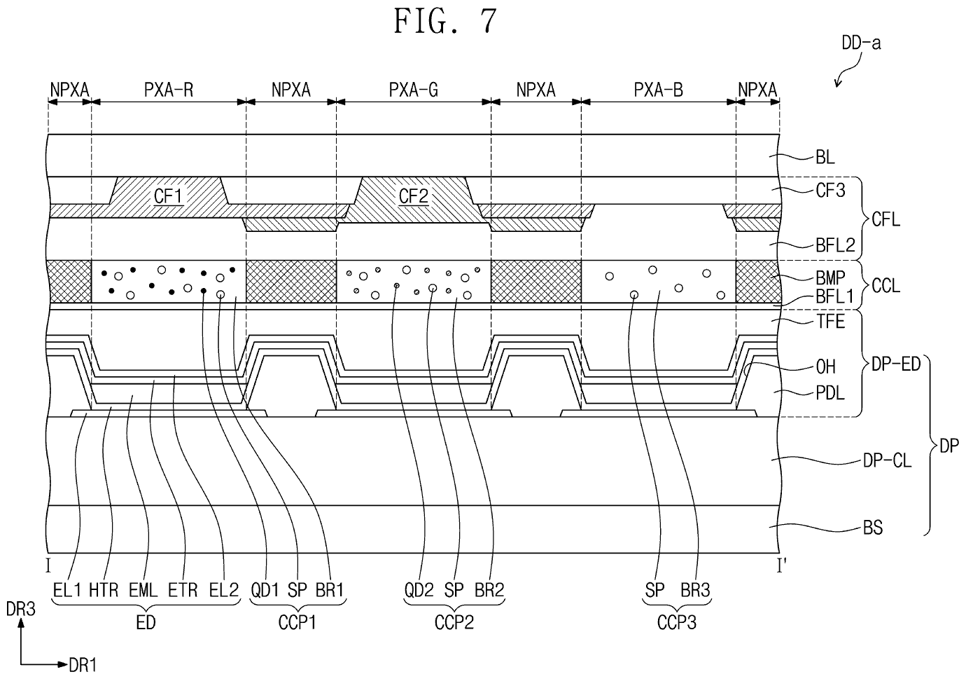

FIGS. 7 to 10 are each a schematic cross-sectional view of a display device according to an embodiment. Hereinafter, in describing the display devices according to embodiments as shown in FIGS. 7 to 10, the features which have been described above with respect to FIGS. 1 to 6 will not be described again, and the differing features will be described.

Referring to FIG. 7, the display device DD-a according to an embodiment may include a display panel DP including a display element layer DP-ED, a light control layer CCL disposed on the display panel DP, and a color filter layer CFL.

In an embodiment shown in FIG. 7, the display panel DP may include a base layer BS, a circuit layer DP-CL provided on the base layer BS, and the display element layer DP-ED, and the display element layer DP-ED may include a light-emitting element ED.

The light-emitting element ED may include a first electrode EL1, a hole transport region HTR disposed on the first electrode EL1, an emission layer disposed on the hole transport region HTR, an electron transport region ETR disposed on the emission layer EML, and a second electrode EL2 disposed on the electron transport region ETR. In embodiments, a structure of the light-emitting element ED shown in FIG. 7 may be the same as a structure of a light-emitting element ED according to one of FIG. 3 to FIG. 6 as described above. The light-emitting element ED illustrated in FIG. 7 may include the amine compound according to an embodiment. The light-emitting element ED that includes the amine compound according to an embodiment may exhibit characteristics of low driving voltage, high efficiency, and long lifespan.

Referring to FIG. 7, the emission layer EML may be disposed in an opening OH defined in a pixel defining film PDL. For example, the emission layer EML, which is separated by the pixel defining film PDL and provided to correspond to each of the light emitting regions PXA-R, PXA-G, and PXA-B, may emit light in a same wavelength range. In the display device DD-a, the emission layer EML may emit blue light. Although not shown in the drawings, in an embodiment, the emission layer EML may be provided as a common layer for each of the light emitting regions PXA-R, PXA-G, and PXA-B.

The light control layer CCL may be disposed on the display panel DP. The light control layer CCL may include a light conversion body. The light conversion body may be a quantum dot, a phosphor, or the like. The light conversion body may convert the wavelength of a provided light and emit the resulting light. For example, the light control layer CCL may be a layer that includes a quantum dot or a layer that includes a phosphor.

The light control layer CCL may include light control parts CCP1, CCP2, and CCP3. The light control parts CCP1, CCP2, and CCP3 may be spaced apart from each other.

Referring to FIG. 7, division patterns BMP may be disposed between the light control parts CCP1, CCP2, and CCP3, which are spaced apart from each other, but embodiments are not limited thereto. FIG. 7 shows that the division patterns BMP do not overlap the light control parts CCP1, CCP2, and CCP3, but the edges of the light control parts CCP1, CCP2, and CCP3 may overlap at least a portion of the division patterns BMP.

The light control layer CCL may include a first light control part CCP1 including a first quantum dot QD1 that converts first color light provided from the light-emitting element ED into second color light, a second light control part CCP2 including a second quantum dot QD2 that converts first color light into third color light, and a third light control part CCP3 that transmits first color light.

In an embodiment, the first light control part CCP1 may provide red light, which is the second color light, and the second light control part CCP2 may provide green light, which is the third color light. The third light control part CCP3 may provide blue light by transmitting the blue light that is the first color light provided from the light-emitting element ED. For example, the first quantum dot QD1 may be a red quantum dot, and the second quantum dot QD2 may be a green quantum dot. The quantum dots QD1 and QD2 may each be a quantum dot as described above.

The light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include a quantum dot but may include the scatterer SP.

The scatterer SP may be inorganic particles. For example, the scatterer SP may include at least one of TiO₂, ZnO, Al₂O₃, SiO₂, and hollow sphere silica. The scatterer SP may include any one of TiO₂, ZnO, Al₂O₃, SiO₂, and hollow sphere silica, or may be a mixture of at least two materials selected from TiO₂, ZnO, Al₂O₃, SiO₂, and hollow sphere silica.

The first light control part CCP1, the second light control part CCP2, and the third light control part CCP3 may include base resins BR1, BR2, and BR3, in which the quantum dots QD1 and QD2 and the scatterer SP are dispersed. In an embodiment, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of various resin compositions, which may be referred to as a binder. For example, the base resins BR1, BR2, and BR3 may be acrylic-based resins, urethane-based resins, silicone-based resins, epoxy-based resins, etc.

The base resins BR1, BR2, and BR3 may each be a transparent resin. In an embodiment, the first base resin BR1, the second base resin BR2, and the third base resin BR3 may be the same as or different from each other.

The light control layer CCL may include a barrier layer BFL1. The barrier layer BFL1 may prevent the penetration of moisture and/or oxygen (hereinafter, referred to as ‘moisture/oxygen’). The barrier layer BFL1 may block the light control parts CCP1, CCP2, and CCP3 from exposure to moisture/oxygen. The barrier layer BFL1 may cover the light control parts CCP1, CCP2, and CCP3. In an embodiment, the barrier layer BFL2 may be provided between the light control parts CCP1, CCP2, and CCP3 and the color filter layer CFL.

The barrier layers BFL1 and BFL2 may each independently include at least one inorganic layer. For example, the barrier layers BFL1 and BFL2 may each independently include an inorganic material. For example, the barrier layers BFL1 and BFL2 may each independently include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film that secures transmittance, etc. The barrier layers BFL1 and BFL2 may each independently further include an organic film. The barrier layers BFL1 and BFL2 may be formed of a single layer or of multiple layers.

In the display device DD-a, the color filter layer CFL may be disposed on the light control layer CCL. In an embodiment, the color filter layer CFL may be directly disposed on the light control layer CCL. For example, the barrier layer BFL2 may be omitted.

The color filter layer CFL may include filters CF1, CF2, and CF3. The color filter layer CFL may include a first filter CF1 that transmits second color light, a second filter CF2 that transmits third color light, and a third filter CF3 that transmits first color light. For example, the first filter CF1 may be a red filter, the second filter CF2 may be a green filter, and the third filter CF3 may be a blue filter. The filters CF1, CF2, and CF3 may each include a polymeric photosensitive resin and a pigment or dye. The first filter CF1 may include a red pigment or dye, the second filter CF2 may include a green pigment or dye, and the third filter CF3 may include a blue pigment or dye.

However, embodiments are not limited thereto, and the third filter CF3 may not include a pigment or dye. The third filter CF3 may include a polymeric photosensitive resin and may not include a pigment or dye. The third filter CF3 may be transparent. The third filter CF3 may be formed of a transparent photosensitive resin.

In an embodiment, the first filter CF1 and the second filter CF2 may each be a yellow filter. The first filter CF1 and the second filter CF2 may not be provided as distinct filters and may be provided as a unitary filter.

Although not shown in the drawings, the color filter layer CFL may further include a light shielding part (not shown). The light shielding part (not shown) may be a black matrix. The light shielding part (not shown) may include an organic light shielding material or an inorganic light shielding material, each including a black pigment or a black dye. The light shielding part (not shown) may prevent light leakage, and may separate boundaries between adjacent filters CF1, CF2, and CF3. In an embodiment, the light shielding part (not shown) may be formed of a blue filter.

The first filter CF1, the second filter CF2, and the third filter CF3 may be disposed to respectively correspond to the red light emitting region PXA-R, the green light emitting region PXA-G, and the blue light emitting region PXA-B.

A base substrate BL may be disposed on the color filter layer CFL. The base substrate BL may provide a base surface on which the color filter layer CFL, the light control layer CCL, and the like are disposed. The base substrate BL may be a glass substrate, a metal substrate, a plastic substrate, etc. However, embodiments are not limited thereto, and the base substrate BL may include an inorganic layer, an organic layer, or a composite material layer. Although not shown in the drawings, in an embodiment, the base substrate BL may be omitted.

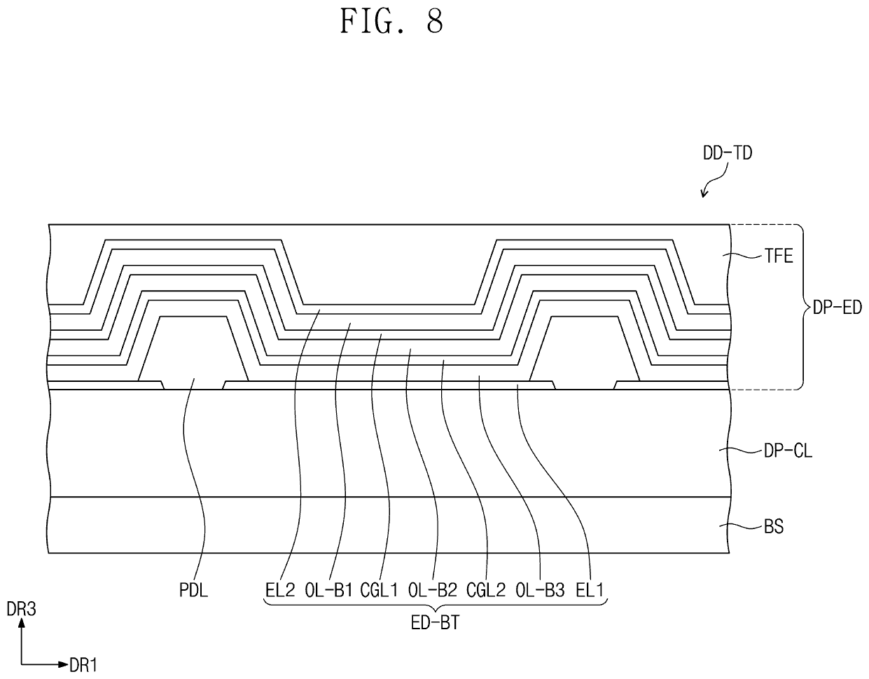

FIG. 8 is a schematic cross-sectional view of a portion of a display device according to an embodiment. In a display device DD-TD according to an embodiment, a light-emitting element ED-BT may include emission structures OL-B1, OL-B2, and OL-B3. The light-emitting element ED-BT may include a first electrode EL1 and a second electrode EL2 that face each other, and emission structures OL-B1, OL-B2, and OL-B3 stacked in a thickness direction between the first electrode EL1 and the second electrode EL2. The emission structures OL-B1, OL-B2, and OL-B3 may each include a hole transport region HTR, an emission layer EML (FIG. 7), and an electron transport region ETR, which may be disposed in that order between the first electrode EL1 and the second electrode EL2. For example, the light-emitting element ED-BT included in the display device DD-TD according to an embodiment may be a light-emitting element having a tandem structure that includes multiple emission layers.

The light-emitting element ED-BT illustrated in FIG. 8 may include an amine compound. The light-emitting element ED-BT that includes the amine compound according to an embodiment may exhibit characteristics of low driving voltage, high efficiency, and long lifespan.

In an embodiment shown in FIG. 8, light emitted from the emission structures OL-B1, OL-B2, and OL-B3 may each be blue light. However, embodiments are not limited thereto, and the light emitted from each of the emission structures OL-B1, OL-B2, and OL-B3 may have wavelength ranges that are different from each other. For example, the light-emitting element ED-BT that includes the emission structures OL-B1, OL-B2, and OL-B3, which emit light in different wavelength ranges from each other, may emit white light.

Charge generation layers CGL1 and CGL2 may each be disposed between two adjacent emission structures among the emission structures OL-B1, OL-B2, and OL-B3. The charge generation layers CGL1 and CGL2 may each independently include a p-type charge generation layer and/or an n-type charge generation layer.

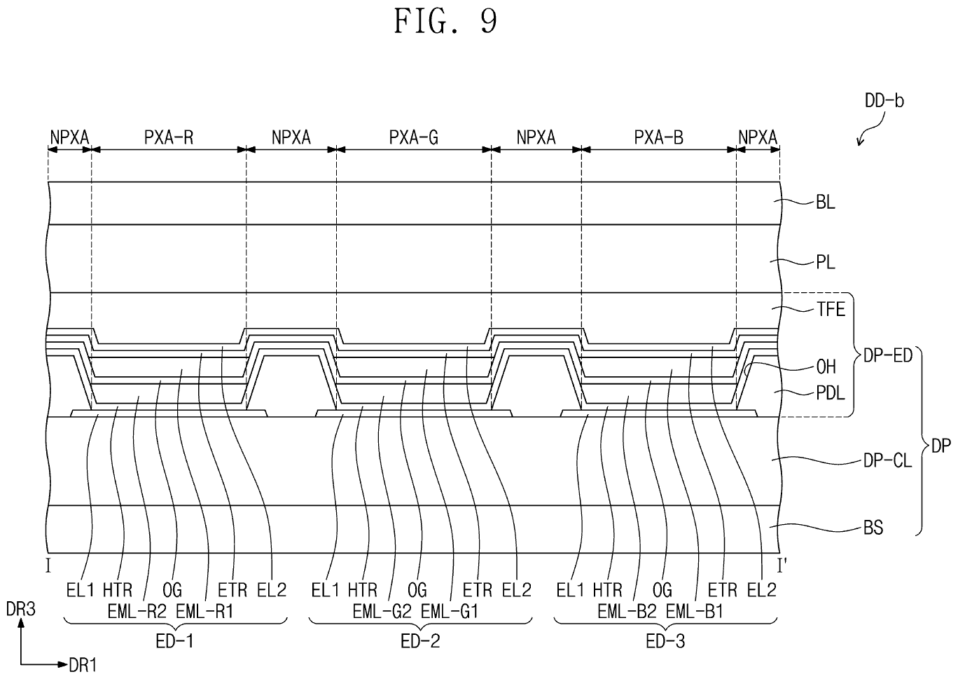

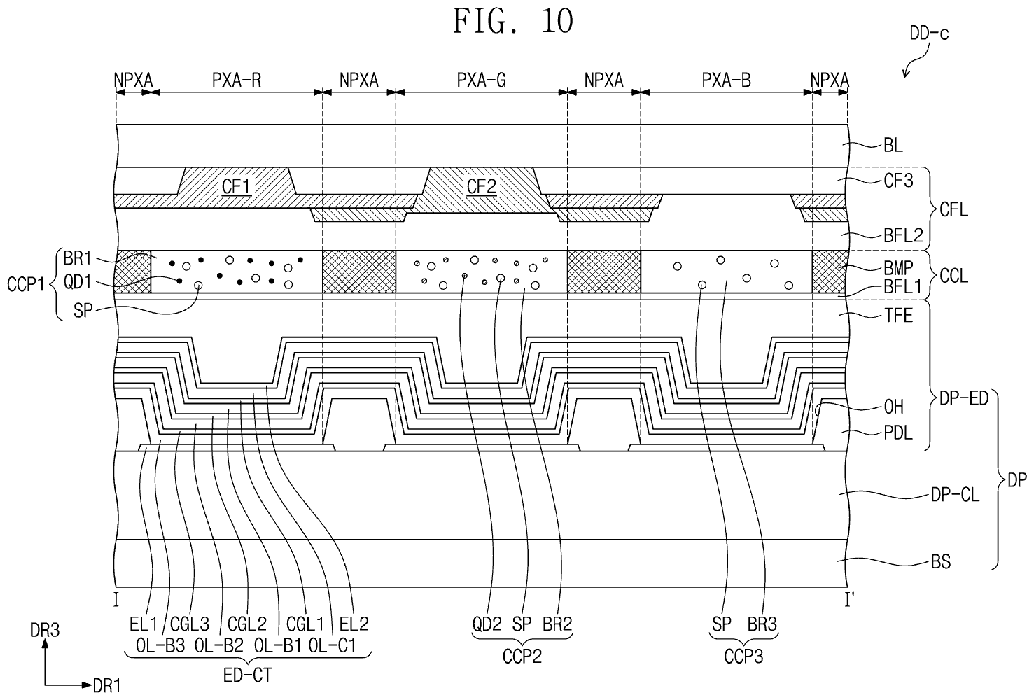

FIG. 9 is a schematic cross-sectional view of a display device DD-b according to an embodiment. FIG. 10 is a schematic cross-sectional view of a display device DD-c according to an embodiment

Referring to FIG. 9, a display device DD-b according to an embodiment may include light-emitting elements ED-1, ED-2, and ED-3 in which two emission layers are stacked. In comparison to the display device DD shown in FIG. 2, the embodiment shown in FIG. 9 is different at least in that the first to third light-emitting elements ED-1, ED-2, and ED-3 each include two emission layers that are stacked in a thickness direction. In each of the first to third light-emitting elements ED-1, ED-2, and ED-3, the two emission layers may emit light in a same wavelength range. At least one of the first to third light-emitting elements ED-1, ED-2, and ED-3 may include the amine compound according to an embodiment. The at least one light-emitting element (at least one of ED-1, ED-2, and ED-3) that includes the amine compound according to an embodiment may exhibit characteristics of low driving voltage, high efficiency, and long lifespan.

The first light-emitting element ED-1 may include a first red emission layer EML-R1 and a second red emission layer EML-R2. The second light-emitting element ED-2 may include a first green emission layer EML-G1 and a second green emission layer EML-G2. The third light-emitting element ED-3 may include a first blue emission layer EML-B1 and a second blue emission layer EML-B2. An emission auxiliary part OG may be disposed between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may have a single-layered structure or a multilayered structure. The emission auxiliary part OG may include a charge generation layer. For example, the emission auxiliary part OG may include an electron transport region, a charge generation layer, and a hole transport region, which may be stacked in that order. The emission auxiliary part OG may be provided as a common layer for each of the first to third light-emitting elements ED-1, ED-2, and ED-3. However, embodiments are not limited thereto, and the emission auxiliary part OG may be provided by being patterned in the openings OH defined in the pixel defining film PDL.

The first red emission layer EML-R1, the first green emission layer EML-G1, and the first blue emission layer EML-B1 may each be disposed between the emission auxiliary part OG and the electron transport region ETR. The second red emission layer EML-R2, the second green emission layer EML-G2, and the second blue emission layer EML-B2 may each be disposed between the hole transport region HTR and the emission auxiliary part OG.

For example, the first light-emitting element ED-1 may include the first electrode EL1, the hole transport region HTR, the second red emission layer EML-R2, the emission auxiliary part OG, the first red emission layer EML-R1, the electron transport region ETR, and the second electrode EL2, which are stacked in that order. The second light-emitting element ED-2 may include the first electrode EL1, the hole transport region HTR, the second green emission layer EML-G2, the emission auxiliary part OG, the first green emission layer EML-G1, the electron transport region ETR, and the second electrode EL2, which are stacked in that order. The third light-emitting element ED-3 may include the first electrode EL1, the hole transport region HTR, the second blue emission layer EML-B2, the emission auxiliary part OG, the first blue emission layer EML-B1, the electron transport region ETR, and the second electrode EL2, which are stacked in that order.

An optical auxiliary layer PL may be disposed on the display element layer DP-ED. The optical auxiliary layer PL may include a polarizing layer. The optical auxiliary layer PL may be disposed on the display panel DP and may control light that is reflected at the display panel DP from an external light. Although not shown in the drawings, in an embodiment, the optical auxiliary layer PL may be omitted from the display device DD-b.

In contrast to FIG. 8 and FIG. 9, FIG. 10 shows a display device DD-c that is different at least in that it includes four emission structures OL-B1, OL-B2, OL-B3, and OL-C1. The light-emitting element ED-CT may include a first electrode EL1 and a second electrode EL2 that face each other, and first to fourth emission structures OL-B1, OL-B2, OL-B3, and OL-C1, which are stacked in a thickness direction between the first electrode EL1 and the second electrode EL2.

Charge generation layers CGL1, CGL2, and CGL3 may each be disposed between two adjacent emission structures among the first to fourth emission structures OL-B1, OL-B2, OL-B3, and OL-C1. The light-emitting element ED-CT illustrated in FIG. 10 may include the amine compound according to an embodiment. The light-emitting element ED-CT that includes the amine compound according to an embodiment may exhibit characteristics of low driving voltage, high efficiency, and long lifespan.

Among the four emission structures, the first to third emission structures OL-B1, OL-B2, and OL-B3 may each emit blue light, and the fourth emission structure OL-C1 may emit green light. However, embodiments are not limited thereto, and the first to fourth emission structures OL-B1, OL-B2, OL-B3, and OL-C1 may each emit light having wavelength ranges that are different from each other.

The charge generation layers CGL1, CGL2, and CGL3 that are disposed between adjacent emission structures among the first to fourth emission structures OL-B1, OL-B2, OL-B3, and OL-C1 may each independently include a p-type charge generation layer and/or an n-type charge generation layer.

In an embodiment, an electronic apparatus may include a display device that includes light-emitting elements, and a control part that controls the display device. The electronic apparatus may be a device that is activated by an electrical signal. The electronic apparatus may include display devices according to various embodiments. Examples of an electronic apparatus may include large electronic apparatuses, such as a television set, a monitor, and an outdoor billboard, and may also include small and medium-sized electronic apparatuses, such as a personal computer, a laptop computer, a personal digital terminal, a display device for a vehicle, a game console, a portable electronic device, and a camera.

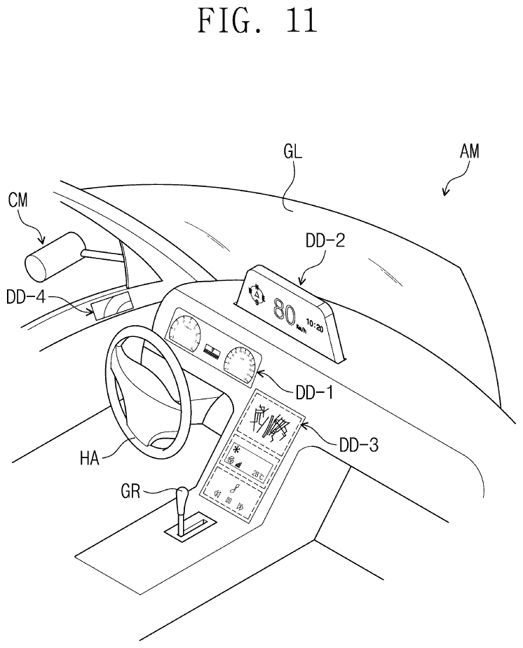

FIG. 11 is a schematic perspective view of a vehicle AM that includes first to fourth display devices DD-1, DD-2, DD-3, and DD-4. At least one of the first to fourth display devices DD-1, DD-2, DD-3, and DD-4 may each have a structure according to one of display devices DD, DD-TD, DD-a, DD-b, and DD-c, as described with reference to FIGS. 1, 2, and 7 to 10.

In FIG. 11, a vehicle AM is shown as an automobile, but this is only an example, and the first to fourth display devices DD-1, DD-2, DD-3, and DD-4 may be disposed in various transportation means, such as bicycles, motorcycles, trains, ships, and aircraft. In an embodiment, at least one of the first to fourth display devices DD-1, DD-2, DD-3, and DD-4, each having a structure according to one of display devices DD, DD-TD, DD-a, DD-b, and DD-c, may be included in a personal computer, a laptop computer, a personal digital terminal, a game console, a portable electronic device, a television, a monitor, a billboard, or the like. However, these are merely listed as examples, and thus may be included in other electronic apparatuses.

At least one of the first to fourth display devices DD-1, DD-2, DD-3, and DD-4 may each independently include a light-emitting element ED according to an embodiment as described with reference to any of FIG. 3 to FIG. 6. At least one of the first to fourth display devices DD-1, DD-2, DD-3, and DD-4 may each independently include the amine compound according to an embodiment. The display device (at least one of DD-1, DD-2, DD-3, and DD-4) that includes the amine compound according to an embodiment may exhibit excellent display efficiency and display lifespan.

Referring to FIG. 11, the vehicle AM may include a steering wheel HA and a gearshift GR for operating the vehicle AM. The vehicle AM may include a front window GL that is disposed so as to face the driver.

The first display device DD-1 may be disposed in a first region that overlaps the steering wheel HA. For example, the first display device DD-1 may be a digital cluster that displays first information of the vehicle AM. The first information may include a first scale that indicates a driving speed of the vehicle AM, a second scale that indicates an engine speed (for example, as revolutions per minute (RPM)), an image that represents a fuel gauge, etc. The first scale and the second scale may each be represented by a digital image.

The second display device DD-2 may be disposed in a second region facing the driver's seat that overlaps the front window GL. The driver's seat may be a seat in which the steering wheel HA is disposed. For example, the second display device DD-2 may be a head up display (HUD) that displays second information of the vehicle AM. The second display device DD-2 may be optically transparent. The second information may include digital numbers that indicate a driving speed of the vehicle AM, and may further include information such as the current time. Although not shown in the drawings, in an embodiment, the second information of the second display device DD-2 may be displayed by being projected onto the front window GL.

The third display device DD-3 may be disposed in a third region that is adjacent to the gearshift GR. For example, the third display device DD-3 may be disposed between the driver's seat and a passenger seat, and may be a center information display (CID) for the vehicle that displays third information. The passenger seat may be a seat that is spaced apart from the driver's seat, and the gear GR may be disposed between the driver's seat and the passenger seat. The third information may include information about traffic or road conditions (for example, navigation information), information about music or radio that is playing, a video (or an image) that is being displayed, information about temperatures inside the vehicle AM, etc.

The fourth display device DD-4 and may be disposed in a fourth region that is spaced apart from the steering wheel HA and the gearshift GR and adjacent to a side of the vehicle AM. For example, the fourth display device DD-4 may be a digital side-view mirror that displays fourth information. The fourth display device DD-4 may display an image that is external to the vehicle AM, which may be taken by a camera module CM disposed at an exterior of the vehicle AM. The fourth information may include an exterior image of the vehicle AM.

The first to fourth information as described above are only provided as examples, and the first to fourth display devices DD-1, DD-2, DD-3, and DD-4 may further display information about the interior and exterior of the vehicle AM. The first to fourth information may include information that is different from each other. However, embodiments are not limited thereto, and a part of the first to fourth information may include a same information as one another.

Hereinafter, an amine compound according to an embodiment and a light-emitting element according to an embodiment will be described with reference to the Examples and the Comparative Examples. The Examples described below are only provided to assist in understanding the disclosure, and the scope thereof is not limited thereto.

### EXAMPLES

### 1. Synthesis of Amine Compounds According to Examples

A synthesis method of an amine compound according to an embodiment will be explained by describing synthesis methods for Compounds A70, A71, A508, A1005, A862, A866, A128, and A178. The synthesis methods of the amine compounds according to the following descriptions are provided only as examples, and the synthesis methods of the amine compound according to embodiments are not limited to the Examples below.

In the synthesis methods of the amine compounds, which will be described below, a molecular weight of each compound was measured by FAB-MS using a JMS-700V (JEOL Ltd.). ¹H-NMR of each compound was measured using an AVAVCE300M spectrometer (Bruker Biospin K.K. Co.), as NMR of the compound.

### (1) Synthesis of Compound A70

Compound A70 according to an embodiment may be synthesized by, for example, Reaction Scheme 1.

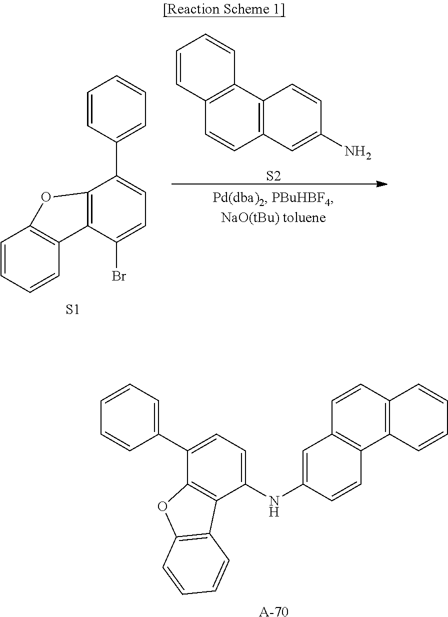

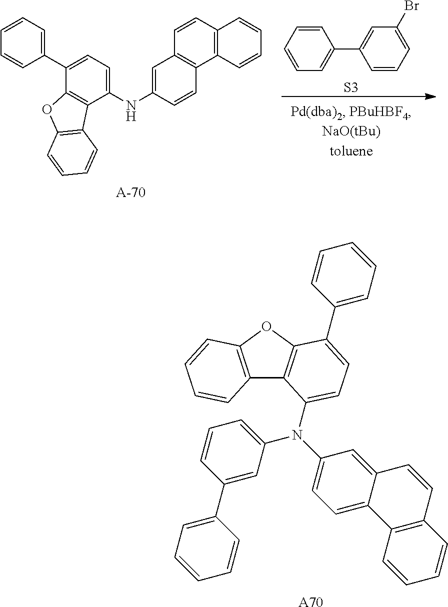

### <Synthesis of Compound A-70>

Under an Ar atmosphere, Compound S1 (10.18 g, 46.41 mmol), Pd(dba)₂ of 1.33 g (0.05 equiv., 2.32 mmol), NaO(tBu) of 4.46 g (1 equiv., 46.41 mmol), toluene of 464 mL, Compound S2 of 13.61 g (1.0 equiv., 46.41 mmol), and PBuHBF₄ of 2.69 g (0.2 equiv., 9.28 mmol) were sequentially added in a 1,000 mL three-necked flask, and the mixture was heated, refluxed, and stirred for about 6 hours. After cooling the reactant to room temperature, water was added to a reaction solvent and an organic layer was separated. Toluene was added to an aqueous layer to additionally separate the organic layer, and the organic layer was collected, washed with saline solution, and dried over MgSO₄. Filtration of MgSO₄ and concentration of the organic layer were conducted, and the obtained crude product was purified by silica gel column chromatography to obtain white solid compound A-70 (17.6 g, yield of 87%). Through FAB-MS, the mass number m/z=435 was measured from the molecular ion peak, confirming that the obtained compound is Compound A-70.

### <Synthesis of Compound A70>

Under an Ar atmosphere, Compound A-70 (9.5 g, 21.67 mmol), Pd(dba)₂ of 0.62 g (0.05 equiv., 1.08 mmol), NaO(tBu) of 2.08 g (1 equiv., 21.67 mmol), toluene of 216 mL, Compound S3 of 5.05 g (1.0 equiv., 21.67 mmol) and PBuHBF₄ of 1.261 g (0.2 equiv., 4.33 mmol) were sequentially added in a 500 mL three-necked flask, and the mixture was heated, refluxed, and stirred for about 6 hours. After cooling the reactant to room temperature, water was added to a reaction solvent and an organic layer was separated. Toluene was added to an aqueous layer to additionally separate the organic layer, and the organic layer was collected, washed with saline solution, and dried over MgSO₄. Filtration of MgSO₄ and concentration of the organic layer were conducted, and the obtained crude product was purified by silica gel column chromatography to obtain white solid compound A70 (8.9 g, yield of 70%). Through FAB-MS, the mass number m/z=587 was measured from the molecular ion peak, confirming that the obtained compound is Compound A70.

### (2) Synthesis of Compound A71

Compound A71 according to an embodiment may be synthesized by, for example, Reaction Scheme 2.

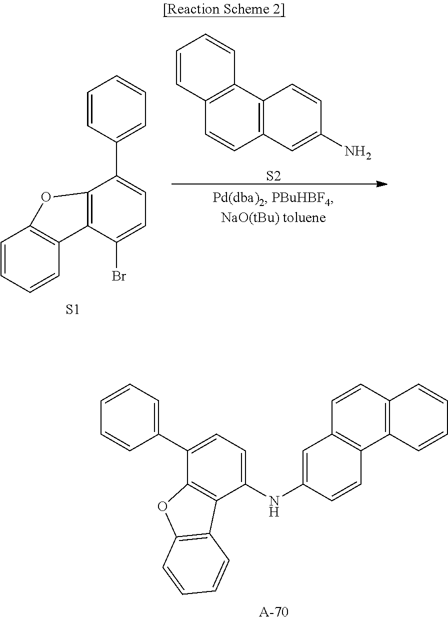

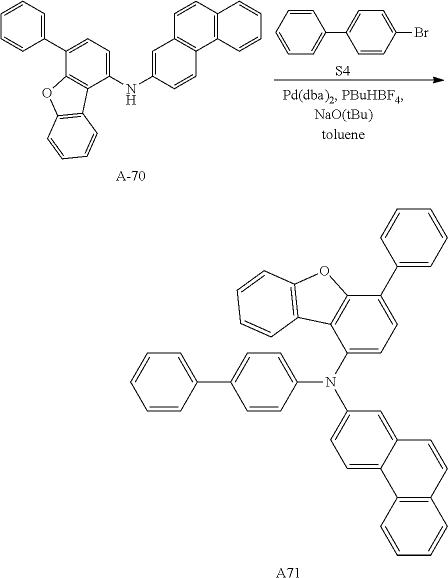

### <Synthesis of Compound A71>

Compound A71 was synthesized in the same manner as the synthesis of Compound A70, except that Compound S4 was used in place of Compound S3. Through FAB-MS, the mass number m/z=587 was measured from the molecular ion peak, confirming that the obtained compound is Compound A71.

### (3) Synthesis of Compound A508

Compound A508 according to an embodiment may be synthesized by, for example, Reaction Scheme 3.

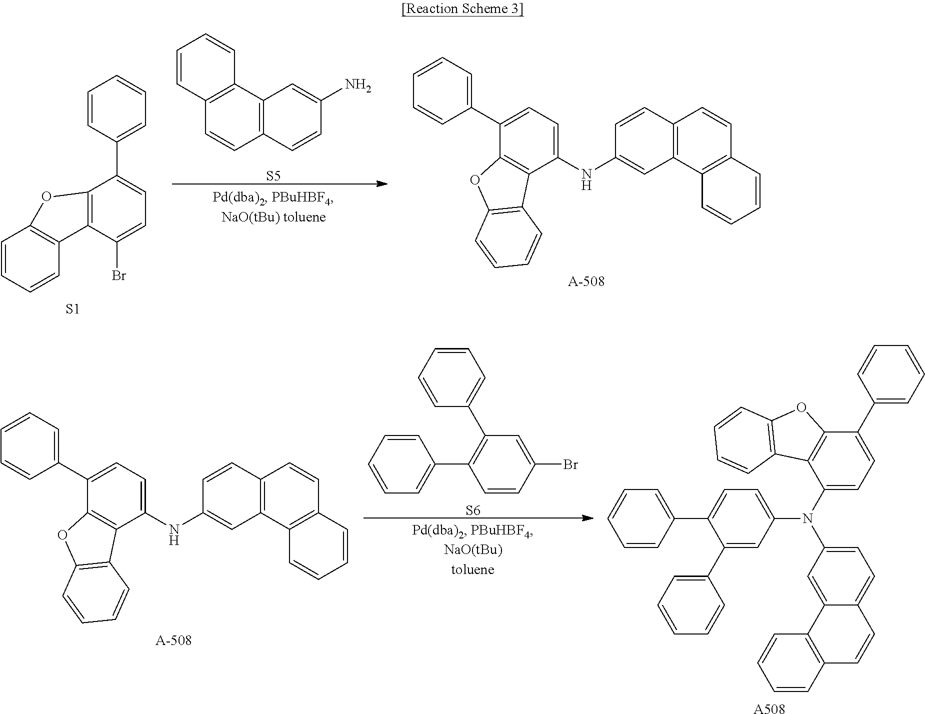

### <Synthesis of Compound A-508>

Compound A-508 was synthesized in the same manner as the synthesis of Compound A-70, except that Compound S5 was used in place of Compound S2.

### <Synthesis of Compound A508>

Compound A508 was synthesized in the same manner as the synthesis of Compound A70, except that Compound A-508 was used in place of Compound A-70 and Compound S6 was used in place of Compound S3. Through FAB-MS, the mass number m/z=663 was measured from the molecular ion peak, confirming that the obtained compound is Compound A508.

### (4) Synthesis of Compound A1005

Compound A1005 according to an embodiment may be synthesized by, for example, Reaction Scheme 4.

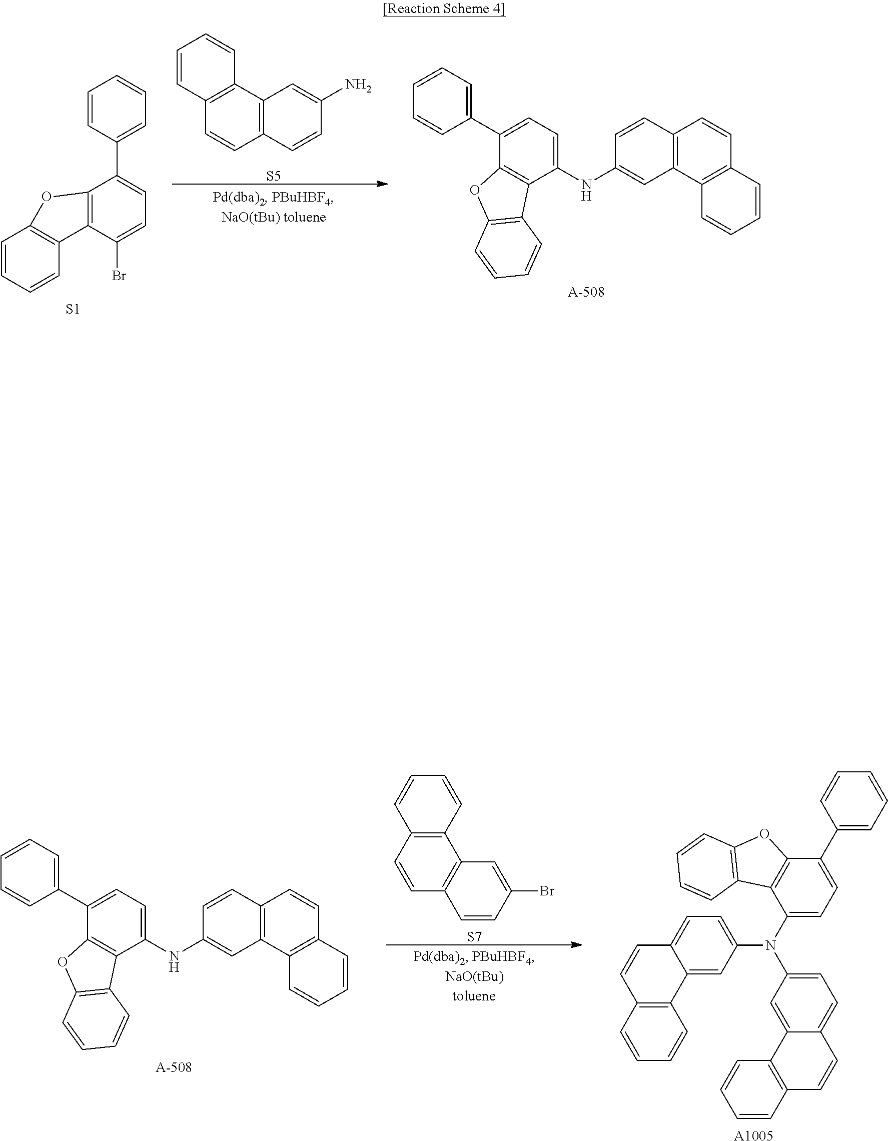

### <Synthesis of Compound A1005>

Compound A1005 was synthesized in the same manner as the synthesis of Compound A70, except that Compound A-508 was used in place of Compound A-70 and Compound S7 was used in place of Compound S3. Through FAB-MS, the mass number m/z=611 was measured from the molecular ion peak, confirming that the obtained compound is Compound A1005.

### (5) Synthesis of Compound A862

Compound A862 according to an embodiment may be synthesized by, for example, Reaction Scheme 5.

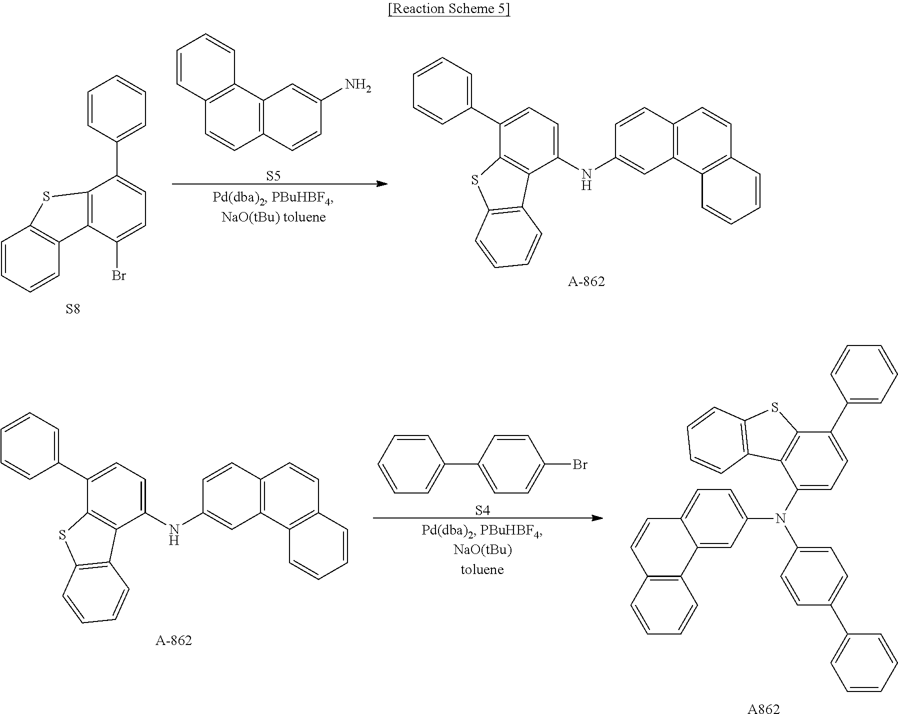

### <Synthesis of Compound A-862>

Compound A-862 was synthesized in the same manner as the synthesis of Compound A-70, except that Compound S5 was used in place of Compound S2.

### <Synthesis of Compound A862>

Compound A862 was synthesized in the same manner as the synthesis of Compound A70, except that Compound A-862 was used in place of Compound A-70 and Compound S4 was used in place of Compound S3. Through FAB-MS, the mass number m/z=603 was measured from the molecular ion peak, confirming that the obtained compound is Compound A862.

### (6) Synthesis of Compound A866

Compound A866 according to an embodiment may be synthesized by, for example, Reaction Scheme 6.

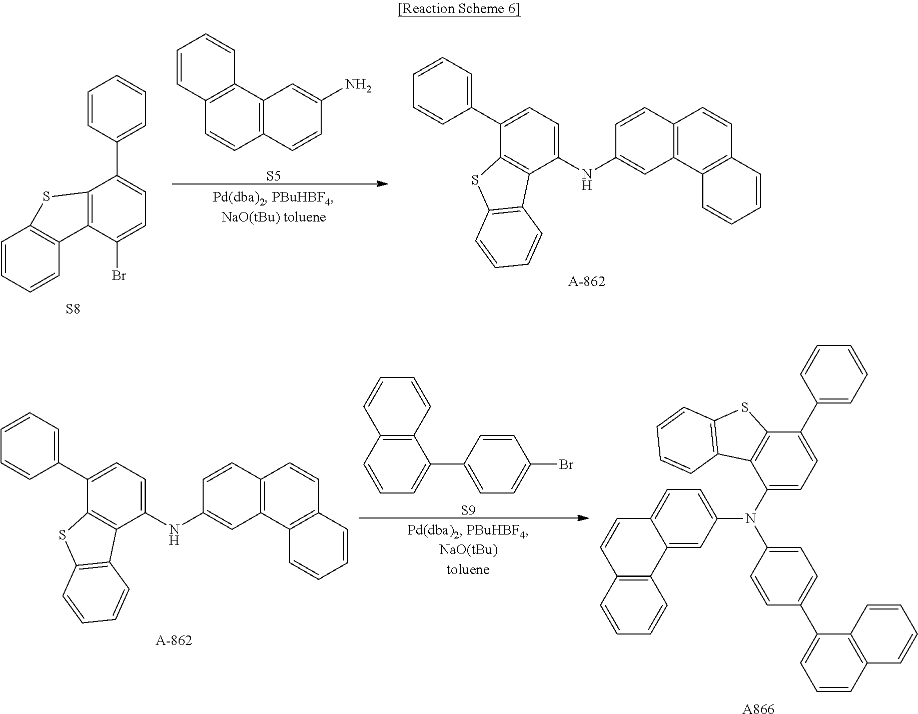

### <Synthesis of Compound A866>

Compound A866 was synthesized in the same manner as the synthesis of Compound A70, except that Compound A-862 was used in place of Compound A-70 and Compound S9 was used in place of Compound S3. Through FAB-MS, the mass number m/z=653 was measured from the molecular ion peak, confirming that the obtained compound is Compound A866.

### (7) Synthesis of Compound A128

Compound A128 according to an embodiment may be synthesized by, for example, Reaction Scheme 7.

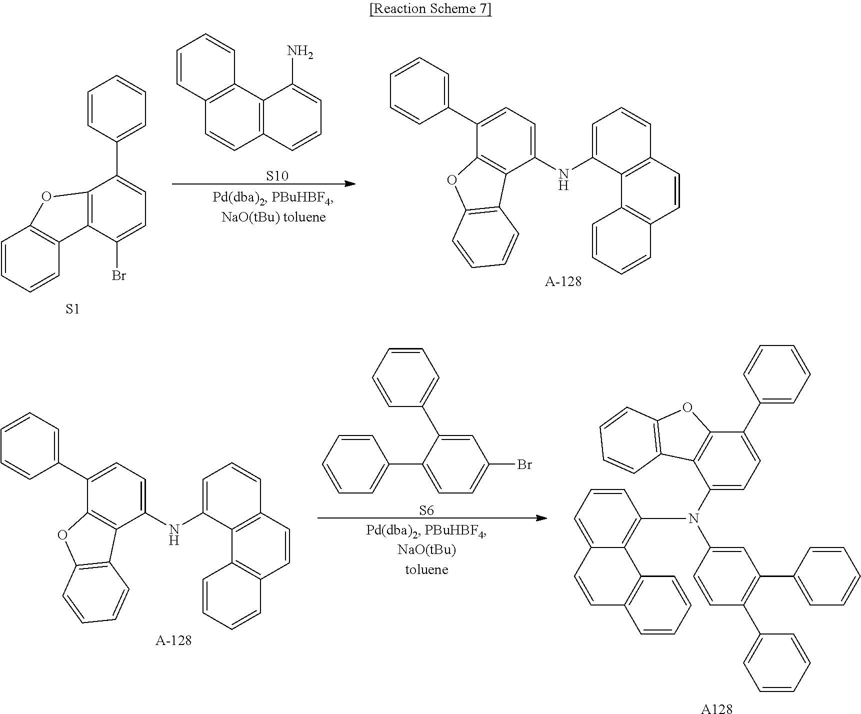

### <Synthesis of Compound A-128>

Compound A-128 was synthesized in the same manner as the synthesis of Compound A-70, except that Compound S10 was used in place of Compound S2.

### <Synthesis of Compound A-128>

Compound A128 was synthesized in the same manner as the synthesis of Compound A70, except that Compound A-128 was used in place of Compound A-70 and Compound S6 was used in place of Compound S3. Through FAB-MS, the mass number m/z=663 was measured from the molecular ion peak, confirming that the obtained compound is Compound A128.

### (8) Synthesis of Compound A178

Compound A178 according to an embodiment may be synthesized by, for example, Reaction Scheme 8.

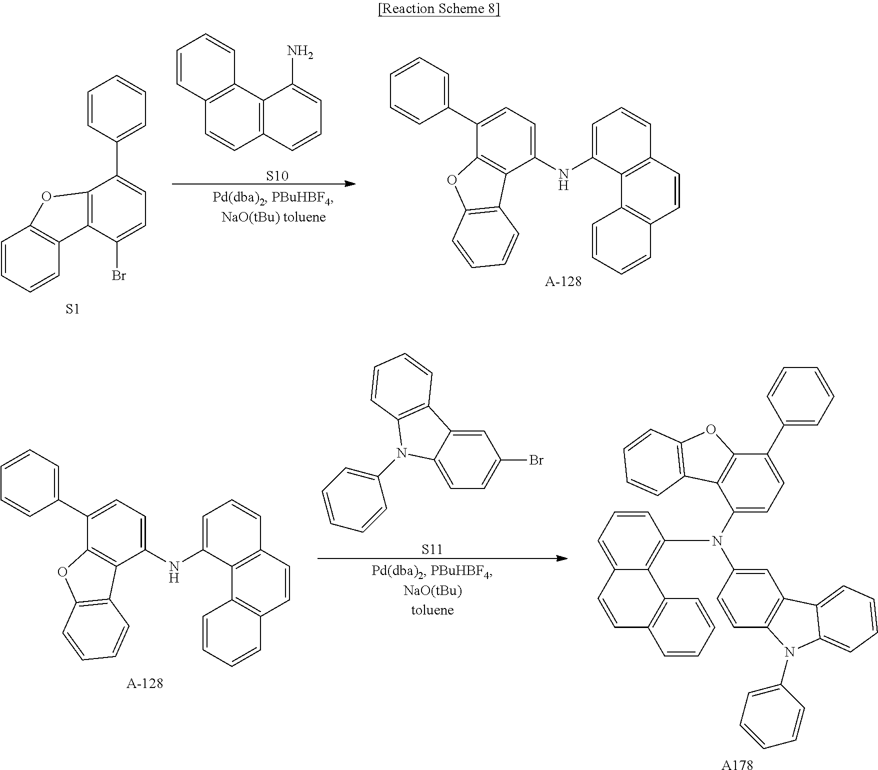

### <Synthesis of Compound A178>

Compound A178 was synthesized in the same manner as the synthesis of Compound A70, except that Compound A-128 was used in place of Compound A-70 and Compound S11 was used in place of Compound S3. Through FAB-MS, the mass number m/z=676 was measured from the molecular ion peak, confirming that the obtained compound is Compound A178.

### 2. Manufacture and Evaluation of Light-Emitting Element

### (1) Manufacture of Light-Emitting Element

Light-emitting elements including the amine compounds according to embodiments or Comparative Example Compounds in the hole transport layer were manufactured in the following manner. Light-emitting elements according to Examples 1 to 8 were manufactured using Compounds A70, A71, A508, A1005, A862, A866, A128, and A178, which are the amine compounds according to embodiments, as materials for the hole transport layer. Light-emitting elements according to Comparative Examples 1 to 11 were manufactured using Comparative Example Compounds CX1 to CX11 as materials for the hole transport layer.

A glass substrate, on which a 150 nm-thick layer of ITO was patterned as a first electrode, was cleansed by ultrasonic waves using isopropyl alcohol and pure water for about five minutes each. After ultrasonic cleansing, the glass substrate was irradiated with UV for about 30 minutes and was subjected to ozone exposure. 1-TNATA was deposited at a thickness of about 60 nm to form a hole injection layer. An Example Compound or a Comparative Example Compound was deposited on the hole injection layer at a thickness of about 30 nm to form a hole transport layer.

TBP and ADN were co-deposited on the hole transport layer at a thickness of about 25 nm to form an emission layer. TBP and ADN were co-deposited at a weight ratio of about 3:97. Alq₃ at a thickness of about 25 nm, and LiF at a thickness of about 1 nm were sequentially deposited to form an electron transport region. Al was deposited at a thickness of 100 nm to form a second electrode. The hole injection layer, the hole transport layer, the emission layer, the electron transport region, and the second electrode were each formed by using a vacuum deposition device.

### <Materials Used in Manufacture of Light-Emitting Elements>

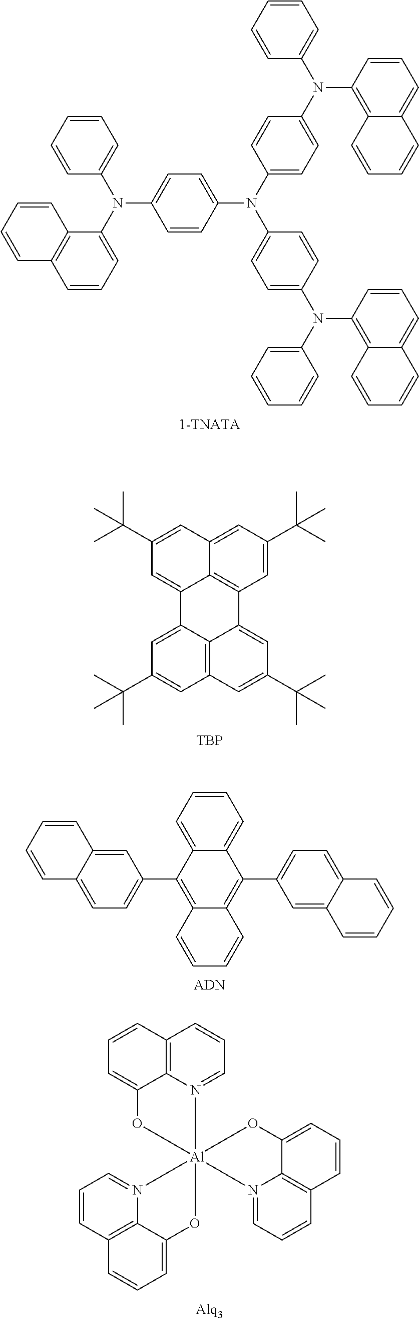

### Example Compounds

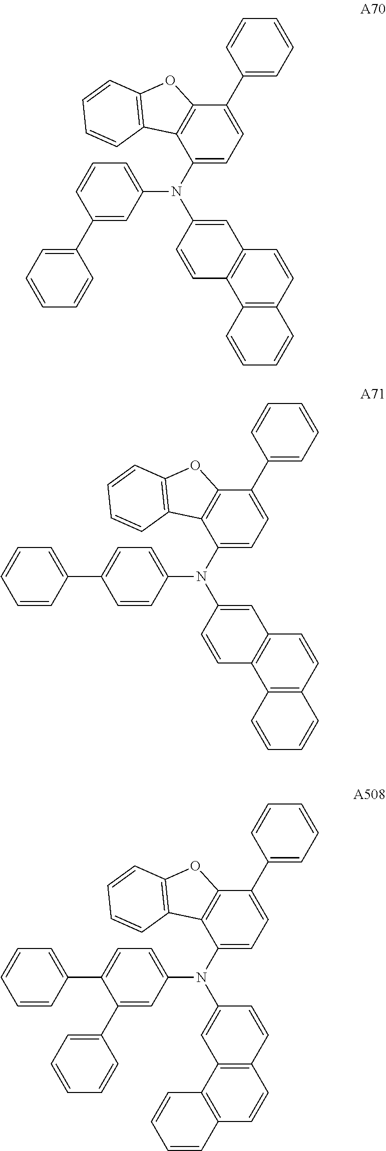

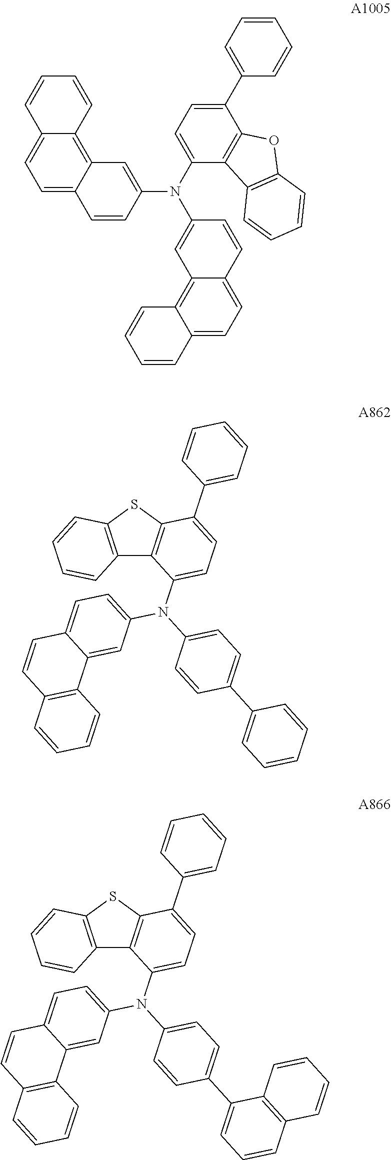 [SMILES: c1ccc(-c2ccc(N(c3ccc4ccc5ccccc5c4c3)c3ccc4ccc5ccccc5c4c3)c3c2oc2ccccc23)cc1 ; c1ccc(-c2ccc(N(c3ccc4ccc5ccccc5c4c3)c3ccc(-c4ccccc4)c4sc5ccccc5c34)cc2)cc1 ; c1ccc(-c2ccc(N(c3ccc(-c4cccc5ccccc45)cc3)c3ccc4ccc5ccccc5c4c3)c3c2sc2ccccc23)cc1]

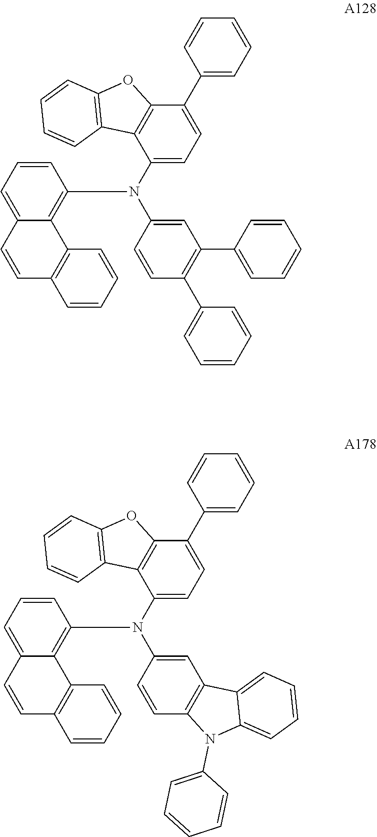 [SMILES: c1ccc(-c2ccc(N(Cc3cccc4ccc5ccccc5c34)c3ccc(-c4ccccc4)c4oc5ccccc5c34)cc2-c2ccccc2)cc1 ; c1ccc(-c2ccc(N(Cc3cccc4ccc5ccccc5c34)c3ccc4c(c3)c3ccccc3n4-c3ccccc3)c3c2oc2ccccc23)cc1]

### Comparative Example Compounds

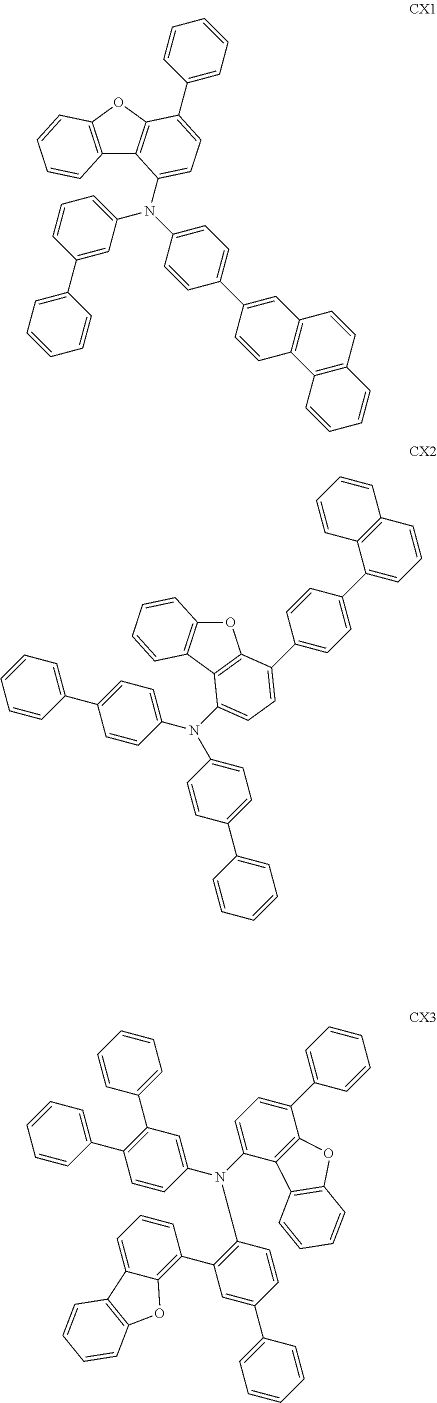

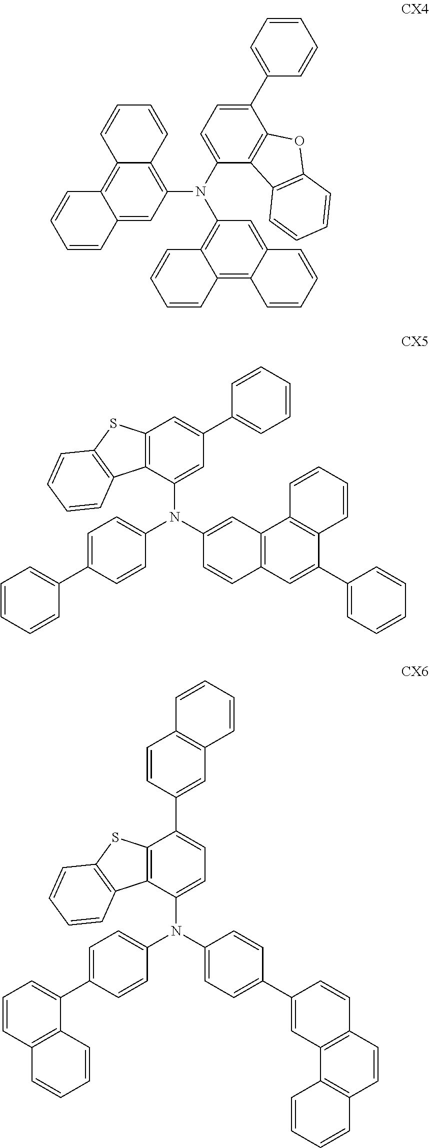 [SMILES: c1ccc(-c2ccc(N(c3cc4ccccc4c4ccccc34)c3cc4ccccc4c4ccccc34)c3c2oc2ccccc23)cc1 ; c1ccc(-c2ccc(N(c3ccc4cc(-c5ccccc5)c5ccccc5c4c3)c3cc(-c4ccccc4)cc4sc5ccccc5c34)cc2)cc1 ; c1ccc2cc(-c3ccc(N(c4ccc(-c5ccc6ccc7ccccc7c6c5)cc4)c4ccc(-c5cccc6ccccc56)cc4)c4c3sc3ccccc34)ccc2c1]

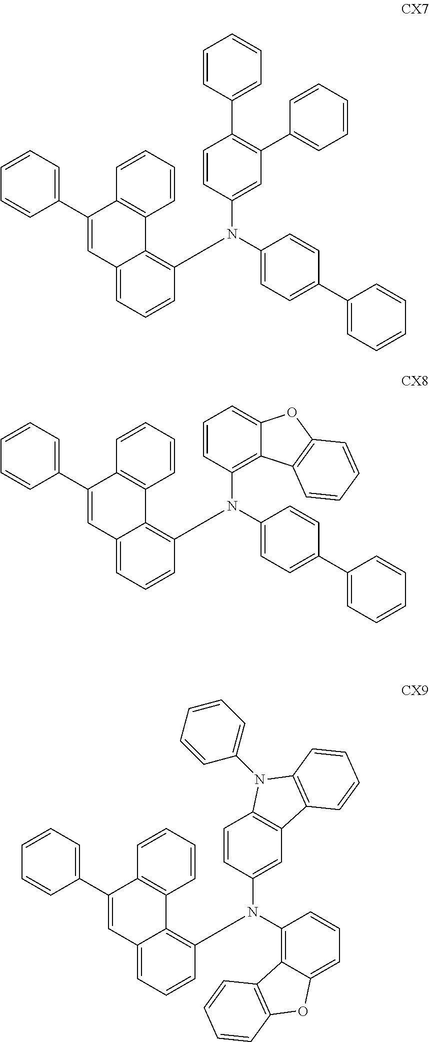 [SMILES: c1ccc(-c2ccc(N(Cc3cccc4cc(-c5ccccc5)c5ccccc5c34)c3ccc(-c4ccccc4)c(-c4ccccc4)c3)cc2)cc1 ; c1ccc(-c2ccc(N(Cc3cccc4cc(-c5ccccc5)c5ccccc5c34)c3cccc4oc5ccccc5c34)cc2)cc1 ; c1ccc(-c2cc3cccc(CN(c4ccc5c(c4)c4ccccc4n5-c4ccccc4)c4cccc5oc6ccccc6c45)c3c3ccccc23)cc1]

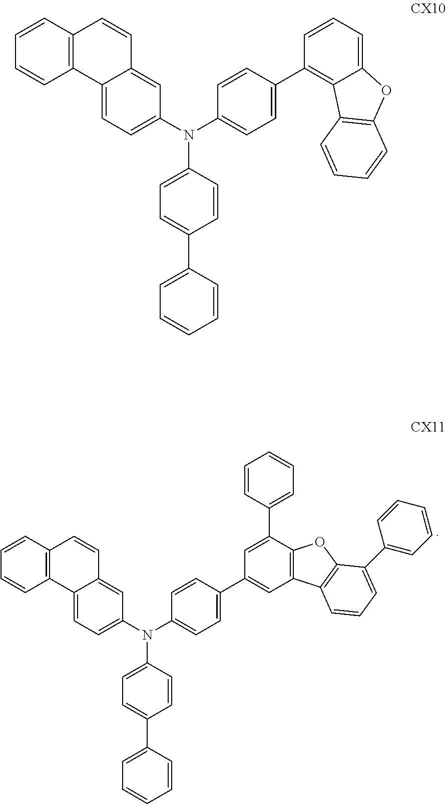 [SMILES: c1ccc(-c2ccc(N(c3ccc(-c4cccc5oc6ccccc6c45)cc3)c3ccc4c(ccc5ccccc54)c3)cc2)cc1 ; c1ccc(-c2ccc(N(c3ccc(-c4cc(-c5ccccc5)c5oc6c(-c7ccccc7)cccc6c5c4)cc3)c3ccc4c(ccc5ccccc54)c3)cc2)cc1]

### (2) Evaluation of Light-Emitting Element

The light-emitting elements according to the Examples and the Comparative Examples were evaluated, and the results are listed in Table 1. Driving voltage, luminous efficiency, and lifespan were measured at a current density of about 10 mA/cm² in a dark room by using a source meter of 2400 series made by Keithley Instruments LLC., a color luminance meter CS-200 made by Konica Minolta, Inc., and LabVIEW 8.2, which is a PC software for measurement made by Japan National Instruments Corporation. Time taken for initial luminance to decrease from 100% to 50% was measured as a lifespan (LT50).

TABLE 1

| | | Driving | Luminous | |
|---|---|---|---|---|
| | | Voltage | efficiency | Lifespan |
| | Hole transport layer | (V) | (cd/A) | (LT50, hr) |
| Example 1 | Compound A70 | 5.4 | 9.5 | 2380 |
| Example 2 | Compound A71 | 5.3 | 9.2 | 2460 |
| Example 3 | Compound A508 | 5.3 | 9.8 | 2550 |
| Example 4 | Compound A1005 | 5.4 | 8.9 | 2500 |
| Example 5 | Compound A862 | 5.5 | 8.7 | 2470 |
| Example 6 | Compound A866 | 5.5 | 9.0 | 2430 |
| Example 7 | Compound A128 | 5.6 | 9.3 | 2450 |
| Example 8 | Compound A178 | 5.2 | 9.1 | 2400 |
| Comparative | Comparative Example | 6.0 | 7.2 | 1300 |
| Example 1 | Compound CX1 | | | |
| Comparative | Comparative Example | 6.0 | 6.2 | 1600 |
| Example 2 | Compound CX2 | | | |
| Comparative | Comparative Example | 6.2 | 7.5 | 1400 |
| Example 3 | Compound CX3 | | | |
| Comparative | Comparative Example | 6.1 | 7.4 | 1500 |
| Example 4 | Compound CX4 | | | |
| Comparative | Comparative Example | 6.2 | 7.2 | 1500 |
| Example 5 | Compound CX5 | | | |
| Comparative | Comparative Example | 6.4 | 6.0 | 1800 |
| Example 6 | Compound CX6 | | | |
| Comparative | Comparative Example | 6.3 | 6.5 | 1700 |
| Example 7 | Compound CX7 | | | |
| Comparative | Comparative Example | 6.2 | 7.1 | 1300 |
| Example 8 | Compound CX8 | | | |
| Comparative | Comparative Example | 5.8 | 7.4 | 1000 |
| Example 9 | Compound CX9 | | | |
| Comparative | Comparative Example | 6.4 | 6.4 | 1200 |
| Example 10 | Compound CX10 | | | |
| Comparative | Comparative Example | 6.7 | 6.8 | 1300 |
| Example 11 | Compound CX11 | | | |

Referring to Table 1, it can be seen that the light-emitting elements according to Examples 1 to 8 each have high luminous efficiency and long lifespan, as compared to the light-emitting elements according to Comparative Examples 1 to 11. It can be seen that the light-emitting elements according to Examples 1 to 8 each have a lower driving voltage than the light-emitting elements according to Comparative Examples 1 to 11. The light-emitting elements according to Examples 1 to 8 include Compounds A70, A71, A508, A1005, A862, A866, A128, and A178, respectively, wherein Compounds A70, A71, A508, A1005, A862, A866, A128, and A178 are amine compounds according to embodiments.

Compounds A70, A71, A508, A1005, A862, A866, A128, and A178 each include dibenzofuran (or dibenzothiophene) and phenanthrene, which are each directly bonded to an amine group.

The dibenzofuran (or dibenzothiophene) corresponds to the above-described first substituent, and the phenanthrene corresponds to the above-described second substituent. Compounds A70, A71, A508, A1005, A862, A866, A128, and A178 each have expanded conjugation systems due to a substitution of a substituent such as a phenyl group at carbon atom number 4 of the first substituent. Therefore, it can be seen that Compounds A70, A71, A508, A1005, A862, A866, A128, and A178 exhibit excellent material stability and hole transporting properties, and the light-emitting elements according to Examples 1 to 8 exhibit characteristics of low driving voltage, high efficiency, and long lifespan.

The light-emitting element according to Comparative Example 1 includes Comparative Example Compound CX1, and the light-emitting element according to Comparative Example 6 includes Comparative Example Compound CX6. Comparative Example Compound CX1 includes dibenzofuran and phenanthrene, and Comparative Example Compound CX6 includes dibenzothiophene and phenanthrene. However, in Comparative Example Compounds CX1 and CX6, phenanthrene is not directly bonded to an amine group and is bonded to the amine group via a phenylene group (corresponding to a linker), which differs from the amine compounds according to embodiments. Thus, for Comparative Example Compounds CX1 and CX6, a deposition temperature of the compound increases, and thermal decomposition becomes more likely to occur during deposition. Therefore, the light-emitting elements according to Comparative Examples 1 and 6 show high driving voltage, low efficiency, and short lifespan.

The light-emitting element according to Comparative Example 2 includes Comparative Example Compound CX2, and the light-emitting element according to Comparative Example 3 includes Comparative Example Compound CX3. Comparative Example Compound CX2 and Comparative Example Compound CX3 each include dibenzofuran but not phenanthrene, which differs from the amine compounds according to embodiments. Therefore, the light-emitting elements according to Comparative Examples 2 and 3 show high driving voltage, low efficiency, and short lifespan.

The light-emitting element according to Comparative Example 4 includes Comparative Example Compound CX4, wherein Comparative Example Compound CX4 includes dibenzofuran and phenanthrene. However, in Comparative Example Compound CX4, a center-disposed ring of phenanthrene is directly bonded to the amine group (a ring group including Rₐ₁ and Rₐ₂ in Formula 2), which differs from the amine compounds according to embodiments. Comparative Example Compound CX4, in which the center-disposed ring of phenanthrene is directly bonded to the amine group, has a large steric distortion around the amine group. Therefore, the light-emitting element according to Comparative Example 4 shows high driving voltage, low efficiency, and short lifespan.

The light-emitting element according to Comparative Example 5 includes Comparative Example Compound CX5, wherein Comparative Example Compound CX5 includes dibenzothiophene and phenanthrene. However, in Comparative Example Compound CX5, a phenyl group that is a substituent of dibenzothiophene is bonded at carbon atom number 3, which differs from the amine compounds according to embodiments. Comparative Example Compound CX5, in which the phenyl group is bonded to the carbon atom number 3 of dibenzothiophene, lacks resonance stabilization. Therefore, the light-emitting element according to Comparative Example 5 shows high driving voltage, low efficiency, and short lifespan.

The light-emitting element according to Comparative Example 7 includes Comparative Example Compound CX7, wherein Comparative Example Compound CX7 includes phenanthrene but not dibenzofuran which differs from the amine compounds according to embodiments. Therefore, the light-emitting element according to Comparative Example 7 shows high driving voltage, low efficiency, and short lifespan.

The light-emitting elements according to Comparative Example 8, Comparative Example 9, and Comparative Example 10 include Comparative Example Compound CX8, CX9, and CX10, respectively. Comparative Example Compounds CX8, CX9, and CX10 each include dibenzofuran and phenanthrene. However, in Comparative Example Compounds CX8, CX9, and CX10, dibenzofuran is unsubstituted, which differs from the amine compounds according to embodiments. Therefore, the light-emitting elements according to Comparative Examples 8, 9, and 10 show high driving voltage, low efficiency, and short lifespan.

The light-emitting element according to Comparative Example 11 includes Comparative Example Compound CX11, wherein Comparative Example Compound CX11 includes dibenzofuran and phenanthrene. However, in Comparative Example Compound CX11, dibenzofuran is bonded to the amine group at carbon atom number 3, which differs from the amine compounds according to embodiments. Therefore, the light-emitting element according to Comparative Example 11 shows high driving voltage, low efficiency, and short lifespan.

The light-emitting element according to an embodiment and the display device according to an embodiment each include the amine compound according to an embodiment, and thus characteristics of high efficiency and long lifespan may be exhibited.

The amine compound according to an embodiment may contribute to improvements in high efficiency and long lifespan.

Embodiments have been disclosed herein, and although terms are employed, they are used and are to be interpreted in a generic and descriptive sense only and not for the purposes of limitation. In some instances, as would be apparent to one of ordinary skill in the art, features, characteristics, and/or elements described in connection with an embodiment may be used singly or in combination with features, characteristics, and/or elements described in connection with other embodiments unless otherwise specifically indicated. Accordingly, it will be understood by those of ordinary skill in the art that various changes in form and details may be made without departing from the spirit and scope of the disclosure as set forth in the claims.

What is claimed is:

1. A light-emitting element comprising:
a first electrode;
a hole transport region disposed on the first electrode;
an emission layer disposed on the hole transport region;
an electron transport region disposed on the emission layer; and
a second electrode disposed on the electron transport region, wherein
the hole transport region includes an amine compound represented by Formula 1: 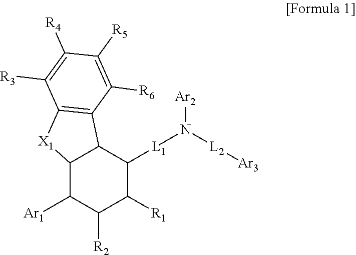
wherein in Formula 1,
X₁ is O or S,
L₁ and L₂ are each independently a direct linkage, a substituted or unsubstituted arylene group having 6 to 15 ring-forming carbon atoms, or a substituted or unsubstituted heteroarylene group having 2 to 15 ring-forming carbon atoms,
Ar₁ is a substituted or unsubstituted aryl group having 6 to 15 ring-forming carbon atoms or a substituted or unsubstituted heteroaryl group having 2 to 15 ring-forming carbon atoms,
Ar₂ is a group represented by Formula 2,
Ar₃ is a substituted or unsubstituted aryl group having 6 to 30 ring-forming carbon atoms or a substituted or unsubstituted heteroaryl group having 2 to 30 ring-forming carbon atoms, and R₁ to R₆ are each independently a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms; 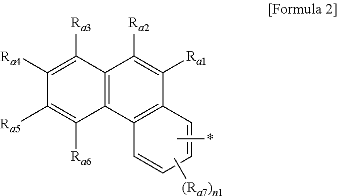
wherein in Formula 2,
n1 is an integer from 0 to 3,
Rₐ₁ to Rₐ₇ are each independently a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms, and [symbol]
represents a bond to a neighboring atom.

2. The light-emitting element of claim 1, wherein the amine compound is represented by one of Formulas 1-B1 to 1-B4: 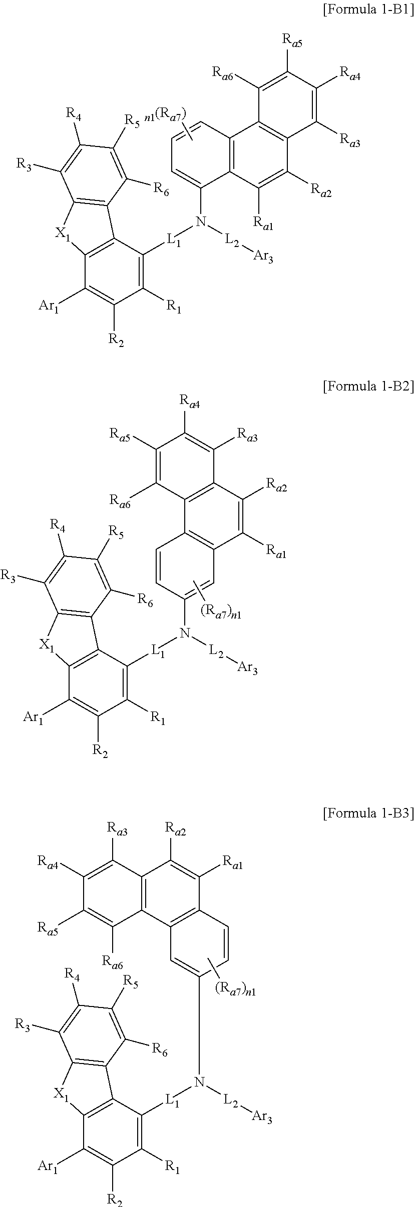 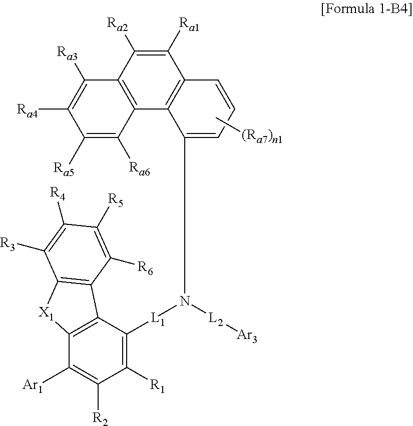
wherein in Formula 1-B1 to Formula 1-B4,
L₁, L₂, Aᵣ₁, Ar₂, Ar₃, R₁ to R₆, Rai to Ray, and n1 are the same as defined in Formulas 1 and 2.

3. The light-emitting element of claim 2, wherein in Formulas 1-B1 to 1-B4, Rai to Rₐ₇ are each independently a hydrogen atom or a deuterium atom.

4. The light-emitting element of claim 1, wherein in Formula 1, An is a group represented by one Formulas AR-1 to AR-4: 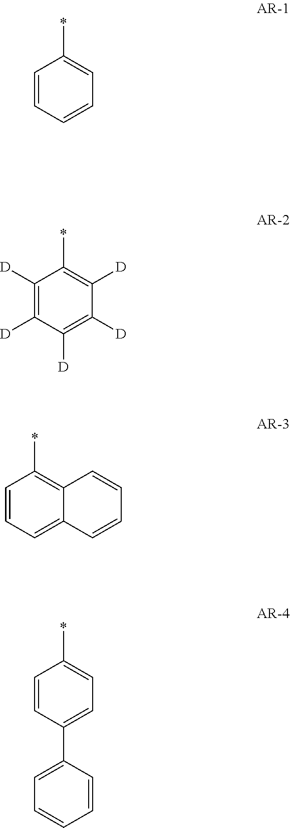 [SMILES: *c1ccccc1 ; *c1c([2H])c([2H])c([2H])c([2H])c1[2H] ; *c1cccc2ccccc12 ; *c1ccc(-c2ccccc2)cc1]
wherein in Formula AR-2,
D is a deuterium atom.

5. The light-emitting element of claim 1, wherein in Formula 1, L₁ is a direct linkage, a substituted or unsubstituted phenylene group, a substituted or unsubstituted naphthylene group, a substituted or unsubstituted divalent dibenzofuran group, or a substituted or unsubstituted divalent dibenzothiophene group.

6. The light-emitting element of claim 1, wherein in Formula 1, Li is a direct linkage or a group represented by one of Formulas L1-1 to Li-5: 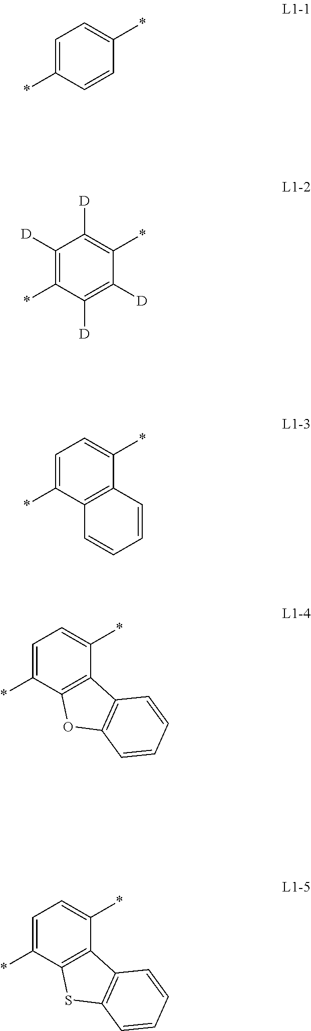 [SMILES: *c1ccc(*)cc1 ; *c1c([2H])c([2H])c(*)c([2H])c1[2H] ; *c1ccc(*)c2ccccc12 ; *c1ccc(*)c2c1oc1ccccc12 ; *c1ccc(*)c2c1sc1ccccc12]
wherein in Formula L1-2,
D is a deuterium atom.

7. The light-emitting element of claim 1, wherein in Formula 1, L₂ is a direct linkage, a substituted or unsubstituted phenylene group, a substituted or unsubstituted biphenylene group, a substituted or unsubstituted naphthylene group, a substituted or unsubstituted divalent carbazole group, a substituted or unsubstituted divalent dibenzofuran group, or a substituted or unsubstituted divalent dibenzothiophene group.

8. The light-emitting element of claim 1, wherein in Formula 1, Ar₃ is a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted carbazole group, a substituted or unsubstituted dibenzofuran group, or substituted or unsubstituted dibenzothiophene group.

9. The light-emitting element of claim 1, wherein in Formula 1, *-L₂-Ar₃ is a moiety represented by one of Formulas LA-1 to LA-79: 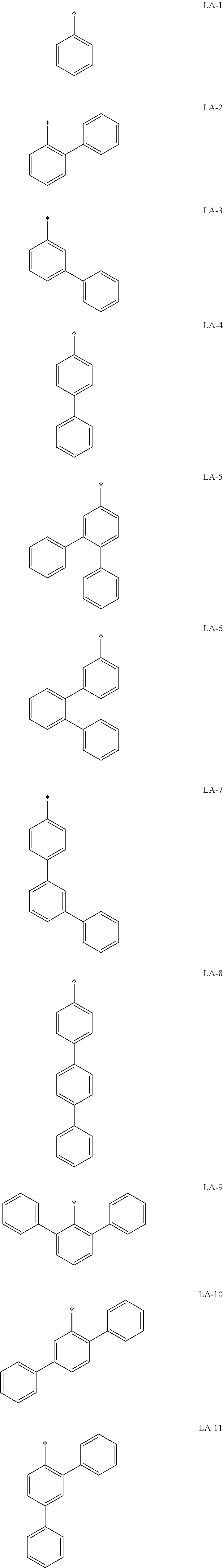 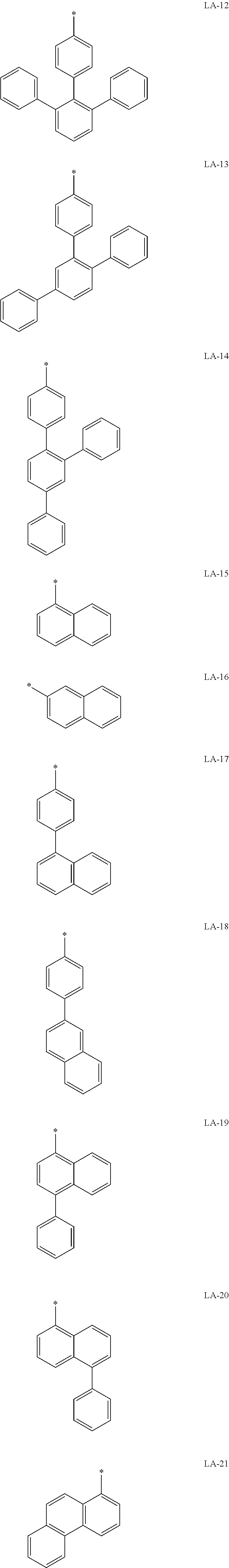 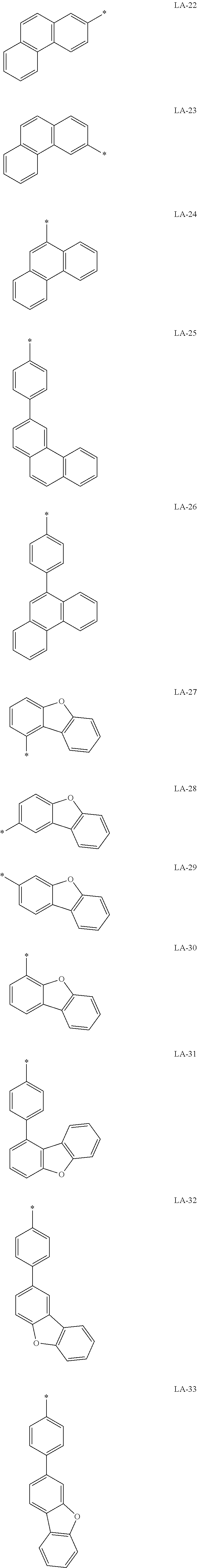 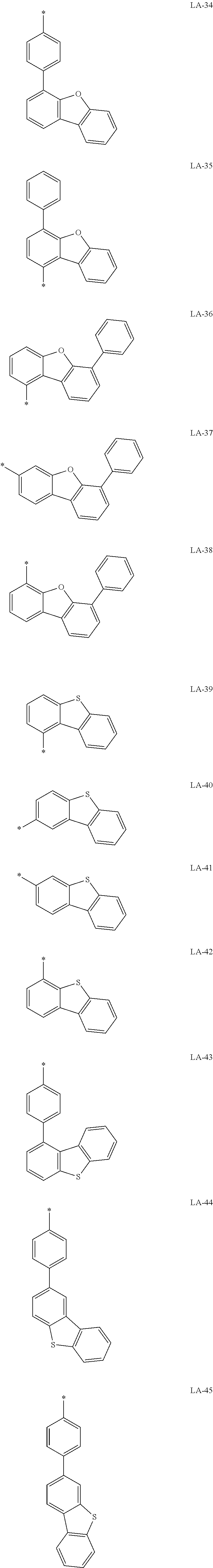 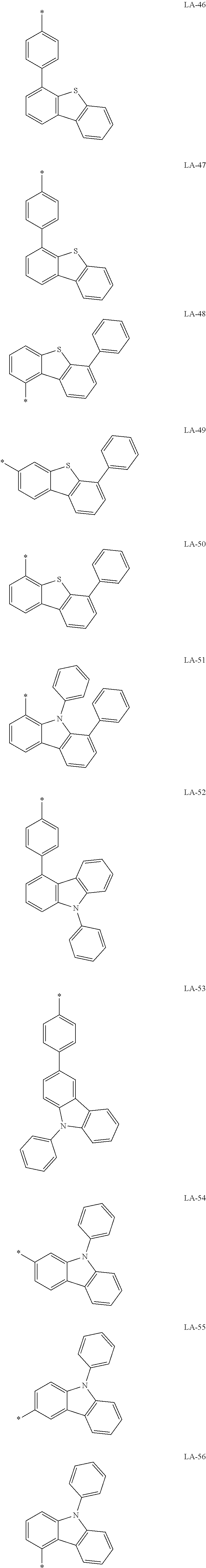 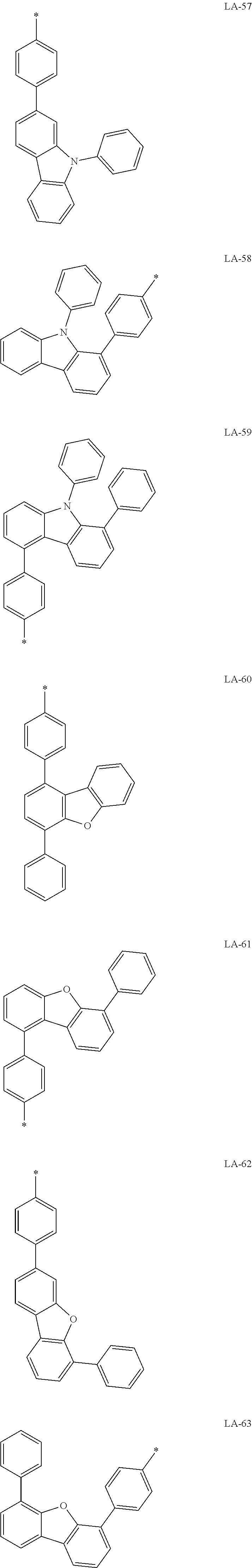 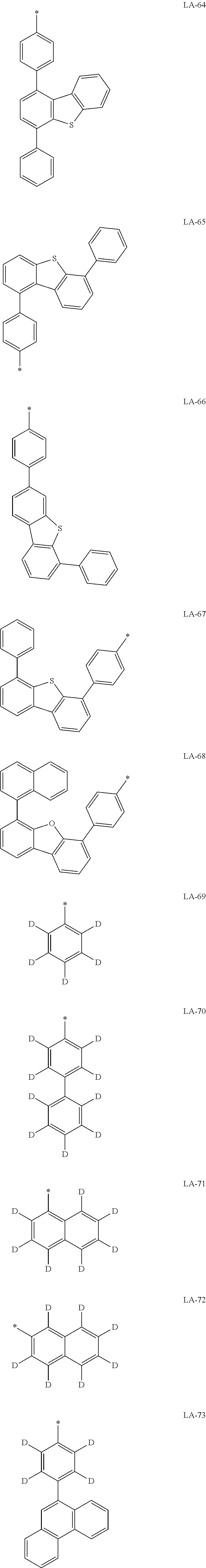 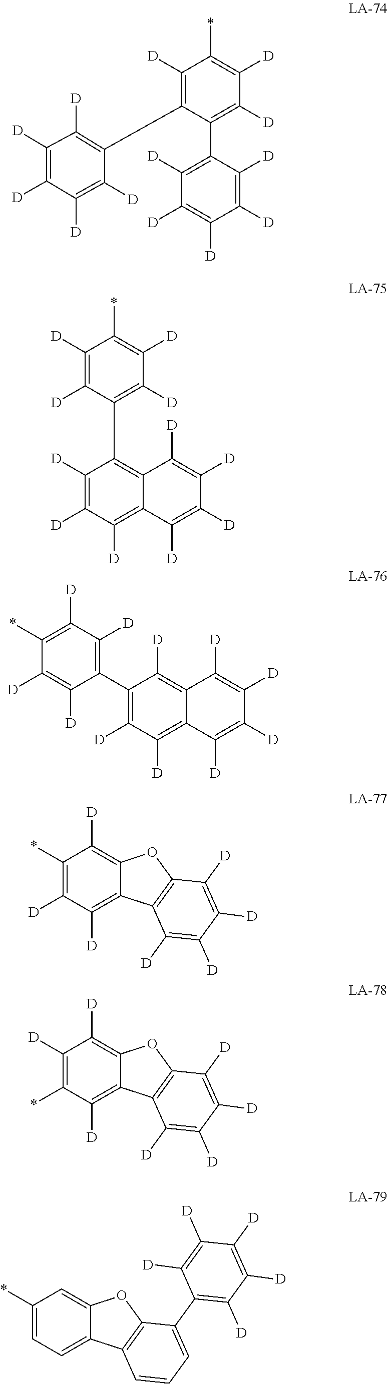 [SMILES: *c1c([2H])c([2H])c(-c2c([2H])c([2H])c([2H])c([2H])c2[2H])c(-c2c([2H])c([2H])c([2H])c([2H])c2[2H])c1[2H] ; *c1c([2H])c([2H])c(-c2c([2H])c([2H])c([2H])c3c([2H])c([2H])c([2H])c([2H])c23)c([2H])c1[2H] ; *c1c([2H])c([2H])c(-c2c([2H])c([2H])c3c([2H])c([2H])c([2H])c([2H])c3c2[2H])c([2H])c1[2H] ; *c1c([2H])c([2H])c2c(oc3c([2H])c([2H])c([2H])c([2H])c32)c1[2H] ; *c1c([2H])c([2H])c2oc3c([2H])c([2H])c([2H])c([2H])c3c2c1[2H] ; *c1ccc2c(c1)oc1c(-c3c([2H])c([2H])c([2H])c([2H])c3[2H])cccc12]
wherein in Formulas LA-69 to LA-79,
D is a deuterium atom.

10. The light-emitting element of claim 1, wherein the amine compound includes at least one compound selected from Compound Group 1: 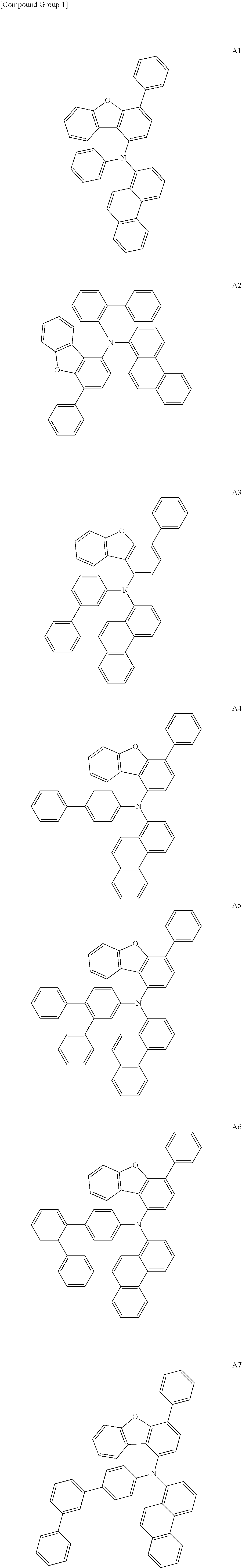 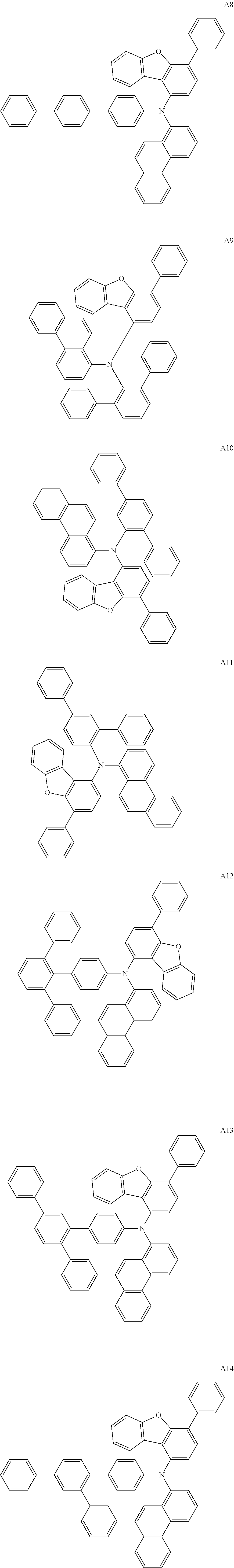 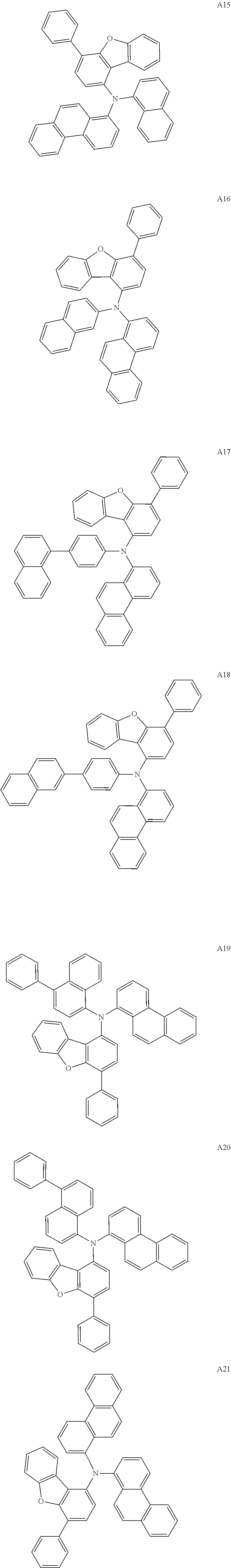 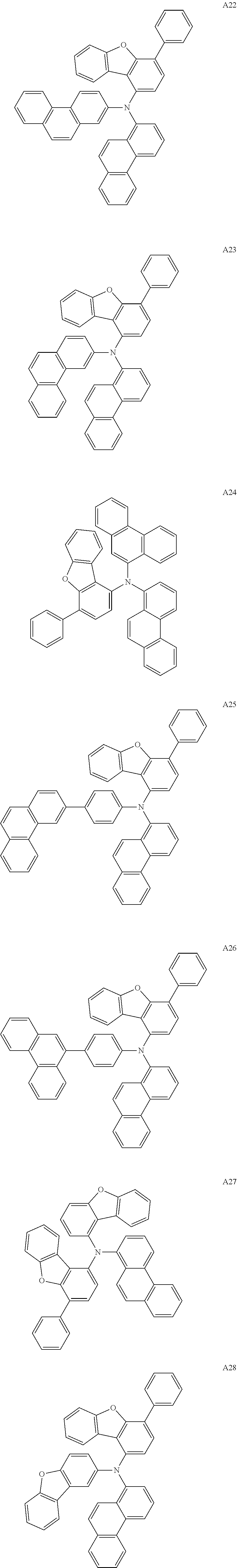 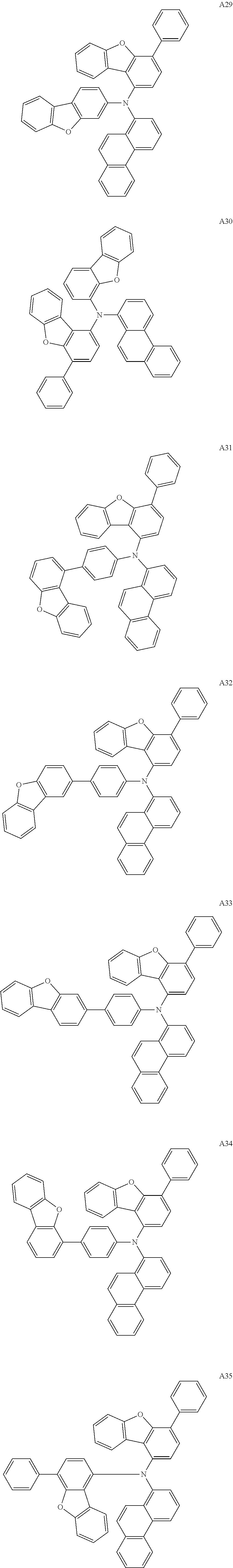 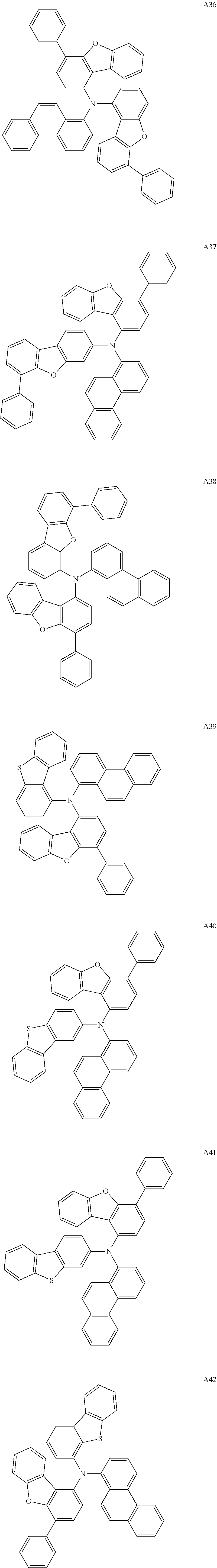 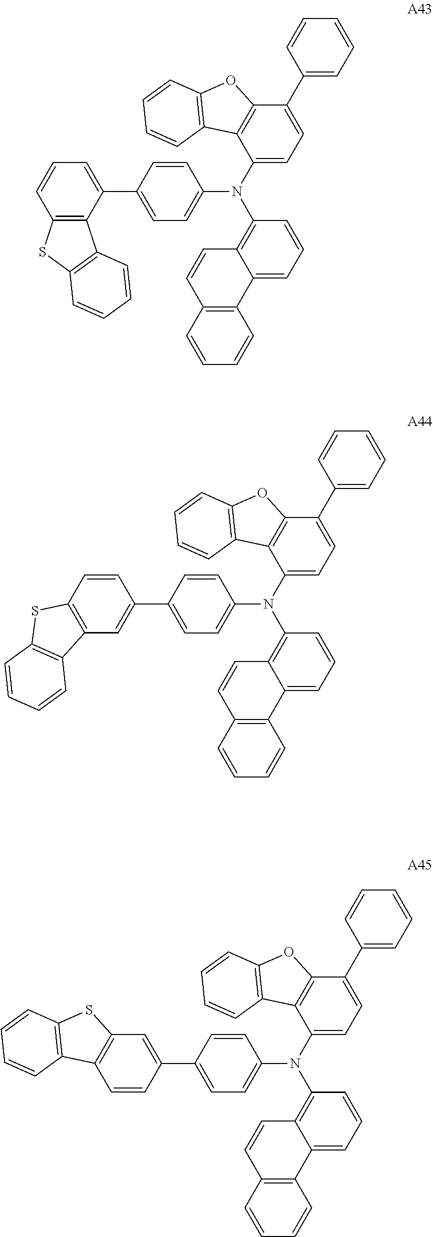 [SMILES: c1ccc(-c2ccc(N(c3ccc(-c4cccc5sc6ccccc6c45)cc3)c3cccc4c3ccc3ccccc34)c3c2oc2ccccc23)cc1 ; c1ccc(-c2ccc(N(c3ccc(-c4ccc5sc6ccccc6c5c4)cc3)c3cccc4c3ccc3ccccc34)c3c2oc2ccccc23)cc1 ; c1ccc(-c2ccc(N(c3ccc(-c4ccc5c(c4)sc4ccccc45)cc3)c3cccc4c3ccc3ccccc34)c3c2oc2ccccc23)cc1] 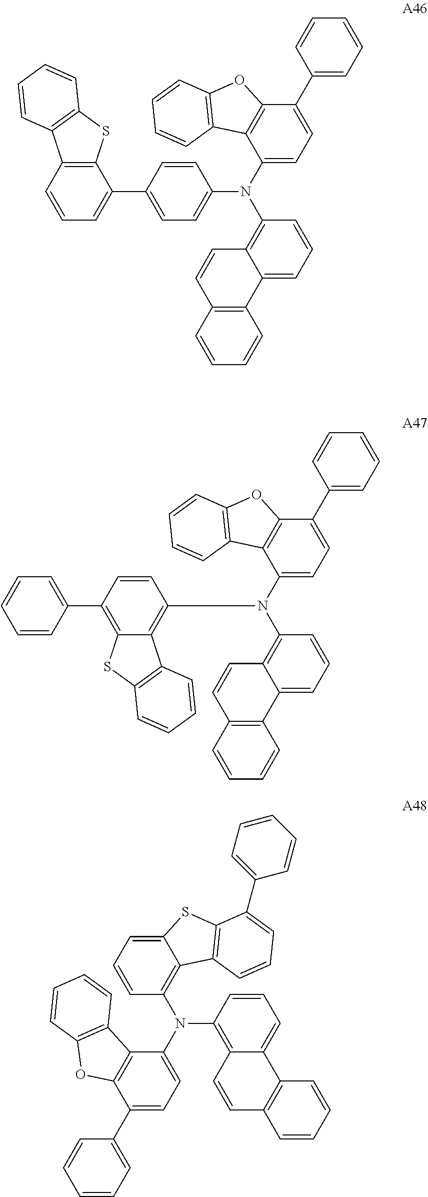 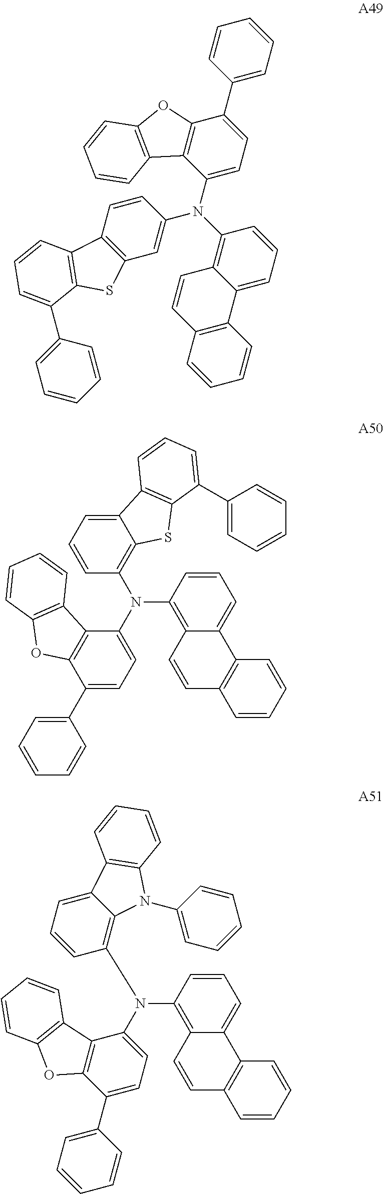 [SMILES: c1ccc(-c2ccc(N(c3ccc4c(c3)sc3c(-c5ccccc5)cccc34)c3cccc4c3ccc3ccccc34)c3c2oc2ccccc23)cc1 ; c1ccc(-c2ccc(N(c3cccc4c3ccc3ccccc34)c3cccc4c3sc3c(-c5ccccc5)cccc34)c3c2oc2ccccc23)cc1 ; c1ccc(-c2ccc(N(c3cccc4c3ccc3ccccc34)c3cccc4c5ccccc5n(-c5ccccc5)c34)c3c2oc2ccccc23)cc1] 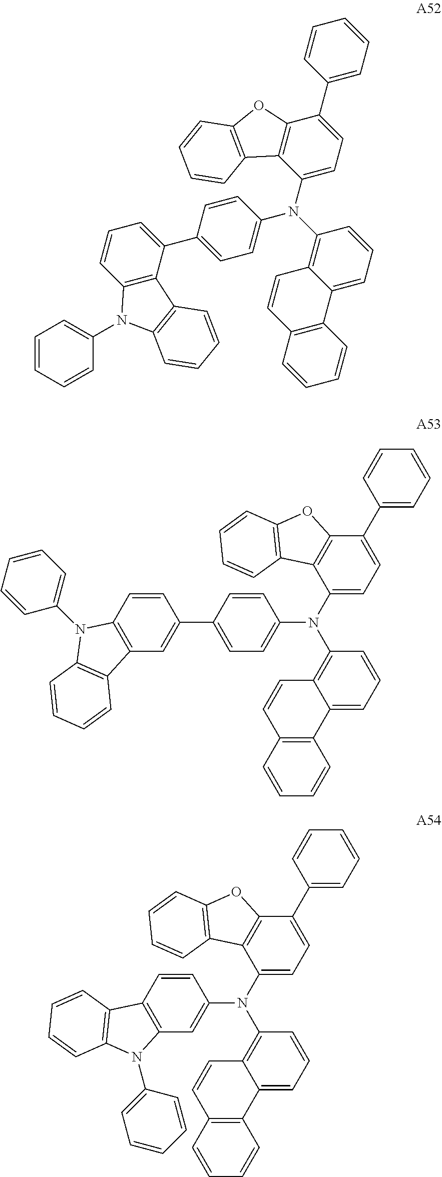 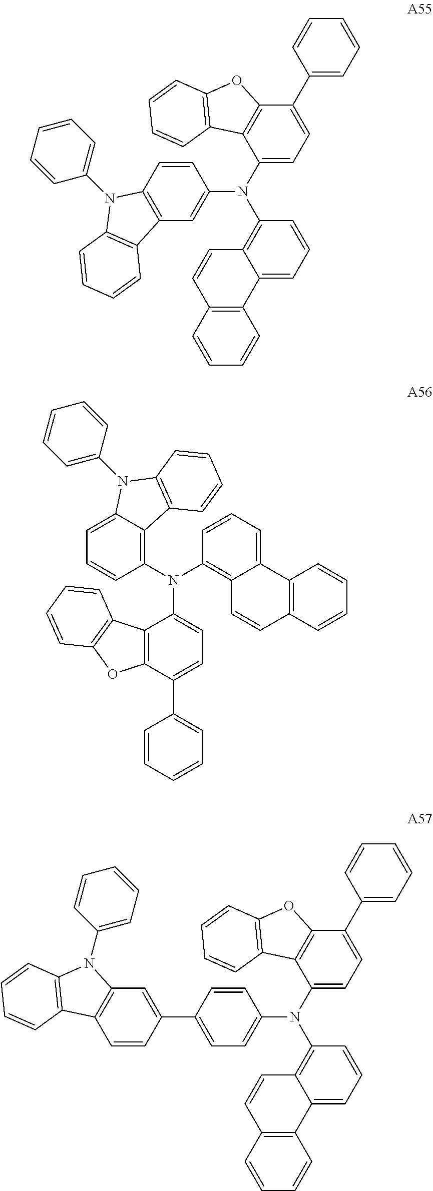 [SMILES: c1ccc(-c2ccc(N(c3ccc4c(c3)c3ccccc3n4-c3ccccc3)c3cccc4c3ccc3ccccc34)c3c2oc2ccccc23)cc1 ; c1ccc(-c2ccc(N(c3cccc4c3ccc3ccccc34)c3cccc4c3c3ccccc3n4-c3ccccc3)c3c2oc2ccccc23)cc1 ; c1ccc(-c2ccc(N(c3ccc(-c4ccc5c6ccccc6n(-c6ccccc6)c5c4)cc3)c3cccc4c3ccc3ccccc34)c3c2oc2ccccc23)cc1] 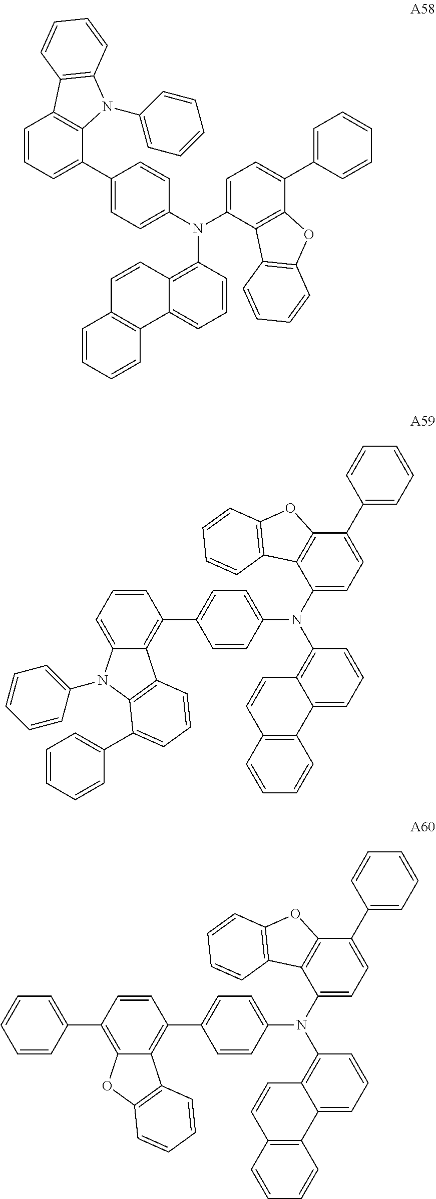 [SMILES: c1ccc(-c2ccc(N(c3ccc(-c4cccc5c6ccccc6n(-c6ccccc6)c45)cc3)c3cccc4c3ccc3ccccc34)c3c2oc2ccccc23)cc1 ; c1ccc(-c2ccc(N(c3ccc(-c4cccc5c4c4cccc(-c6ccccc6)c4n5-c4ccccc4)cc3)c3cccc4c3ccc3ccccc34)c3c2oc2ccccc23)cc1 ; c1ccc(-c2ccc(-c3ccc(N(c4cccc5c4ccc4ccccc45)c4ccc(-c5ccccc5)c5oc6ccccc6c45)cc3)c3c2oc2ccccc23)cc1] 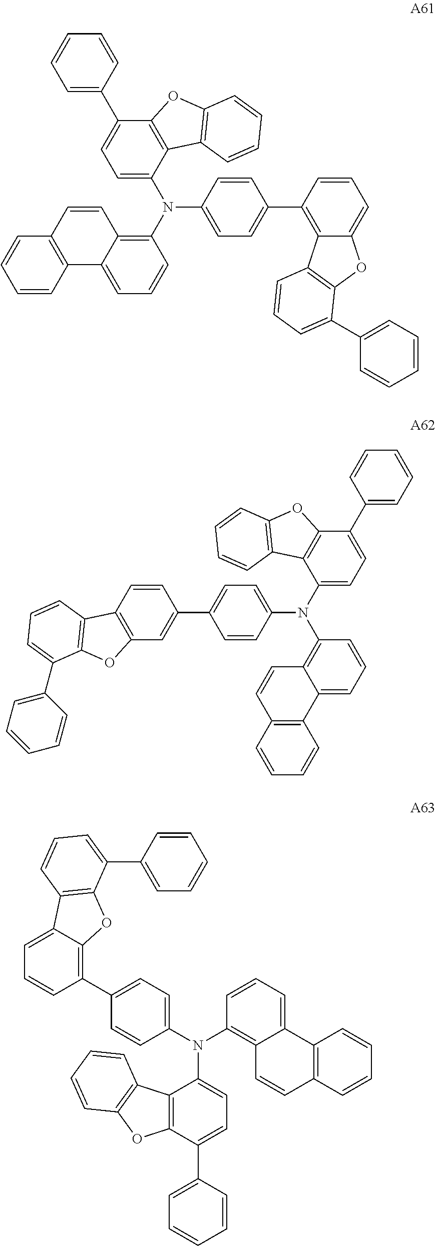 [SMILES: c1ccc(-c2cccc3c2oc2cccc(-c4ccc(N(c5cccc6c5ccc5ccccc56)c5ccc(-c6ccccc6)c6oc7ccccc7c56)cc4)c23)cc1 ; c1ccc(-c2cccc3c2oc2cc(-c4ccc(N(c5cccc6c5ccc5ccccc56)c5ccc(-c6ccccc6)c6oc7ccccc7c56)cc4)ccc23)cc1 ; c1ccc(-c2cccc3c2oc2c(-c4ccc(N(c5cccc6c5ccc5ccccc56)c5ccc(-c6ccccc6)c6oc7ccccc7c56)cc4)cccc23)cc1] 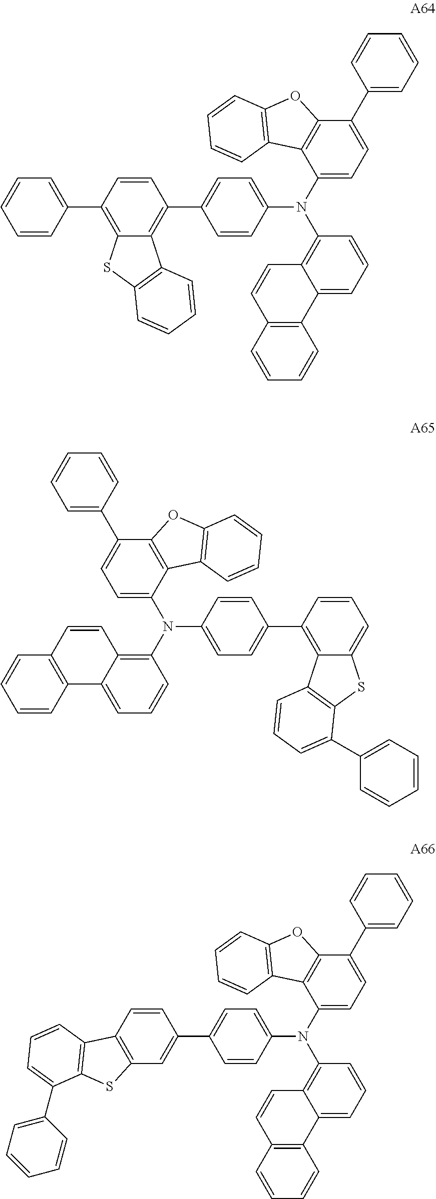 [SMILES: c1ccc(-c2ccc(N(c3ccc(-c4ccc(-c5ccccc5)c5sc6ccccc6c45)cc3)c3cccc4c3ccc3ccccc34)c3c2oc2ccccc23)cc1 ; c1ccc(-c2ccc(N(c3ccc(-c4cccc5sc6c(-c7ccccc7)cccc6c45)cc3)c3cccc4c3ccc3ccccc34)c3c2oc2ccccc23)cc1 ; c1ccc(-c2ccc(N(c3ccc(-c4ccc5c(c4)sc4c(-c6ccccc6)cccc45)cc3)c3cccc4c3ccc3ccccc34)c3c2oc2ccccc23)cc1] 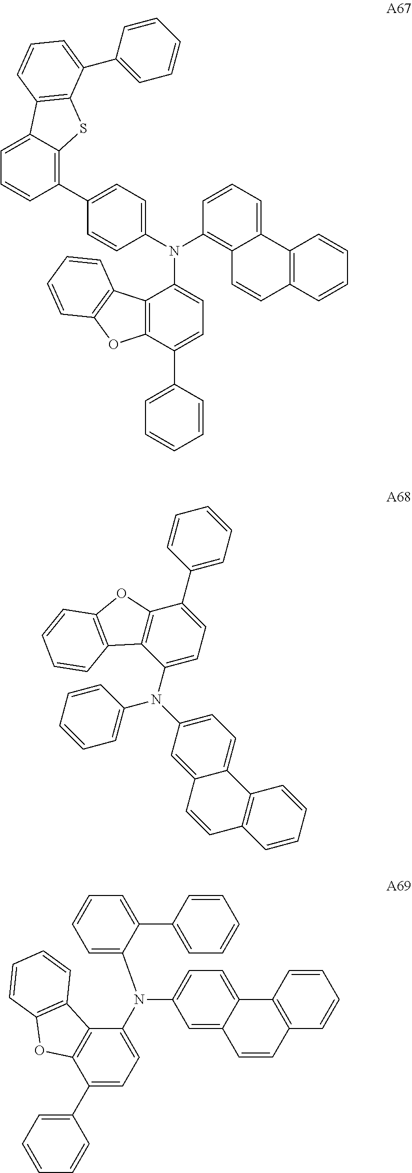 [SMILES: c1ccc(-c2ccc(N(c3ccc(-c4cccc5c4sc4c(-c6ccccc6)cccc45)cc3)c3cccc4c3ccc3ccccc34)c3c2oc2ccccc23)cc1 ; c1ccc(-c2ccc(N(c3ccccc3)c3ccc4c(ccc5ccccc54)c3)c3c2oc2ccccc23)cc1 ; c1ccc(-c2ccccc2N(c2ccc3c(ccc4ccccc43)c2)c2ccc(-c3ccccc3)c3oc4ccccc4c23)cc1] 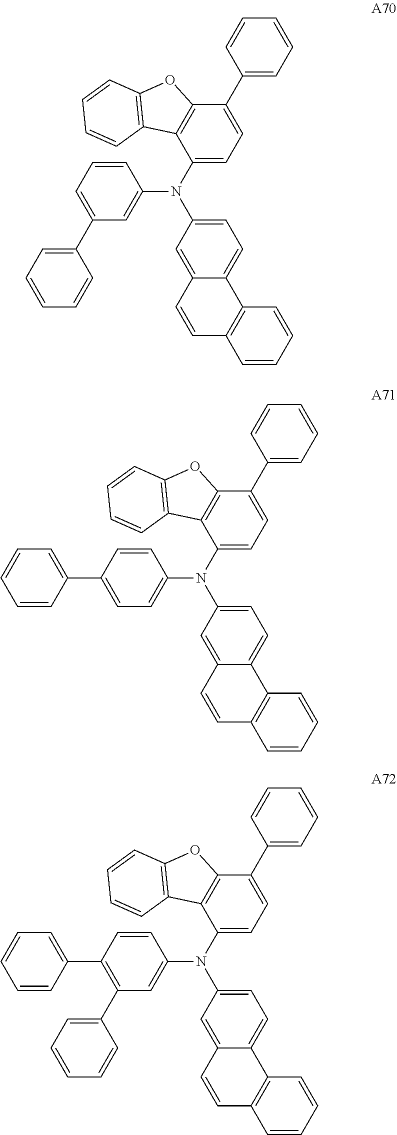 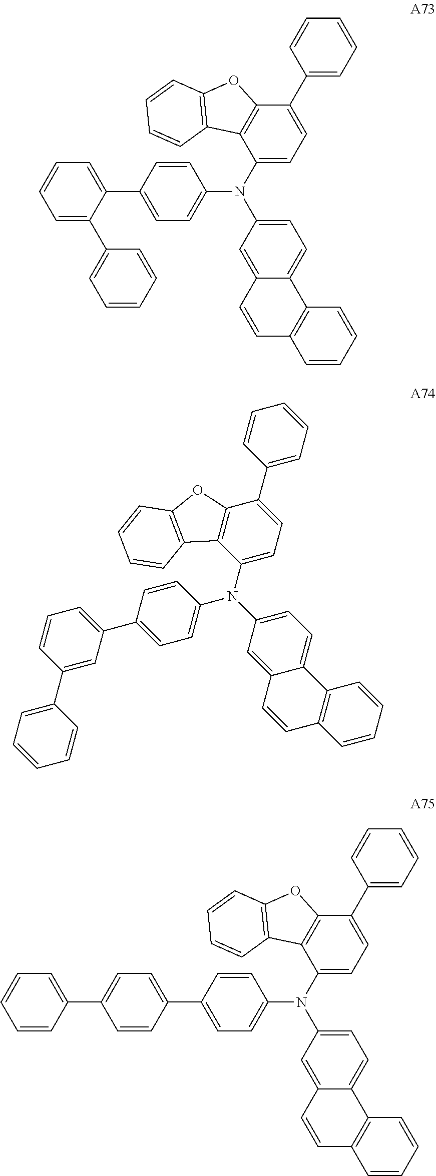 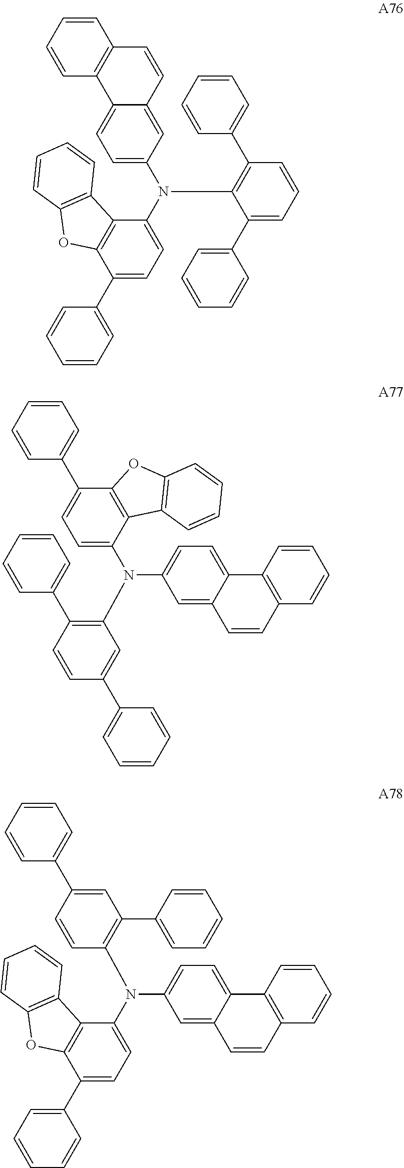 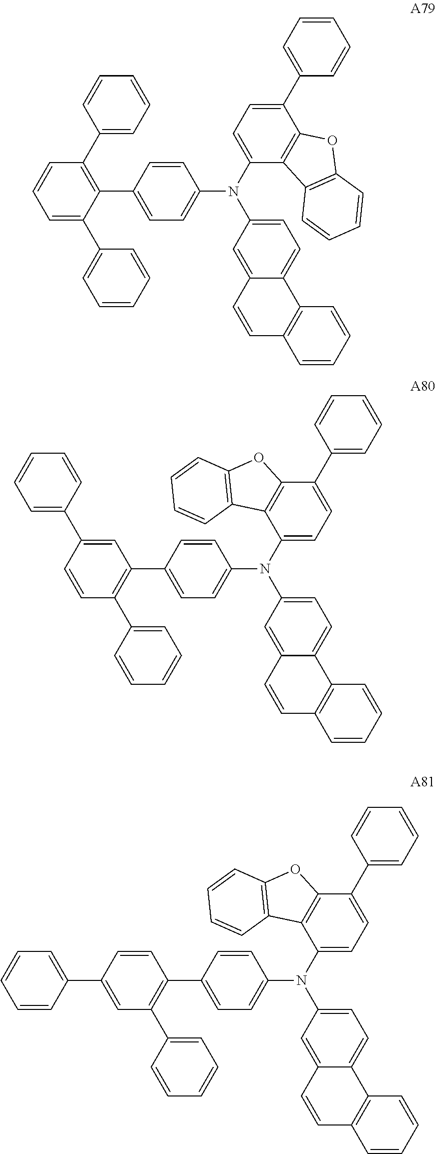 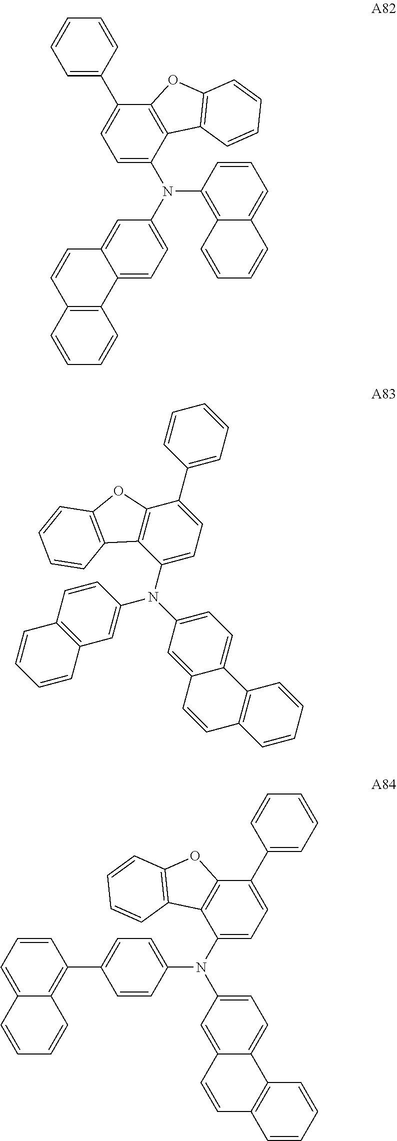 [SMILES: c1ccc(-c2ccc(N(c3ccc4c(ccc5ccccc54)c3)c3cccc4ccccc34)c3c2oc2ccccc23)cc1 ; c1ccc(-c2ccc(N(c3ccc4ccccc4c3)c3ccc4c(ccc5ccccc54)c3)c3c2oc2ccccc23)cc1 ; c1ccc(-c2ccc(N(c3ccc(-c4cccc5ccccc45)cc3)c3ccc4c(ccc5ccccc54)c3)c3c2oc2ccccc23)cc1] 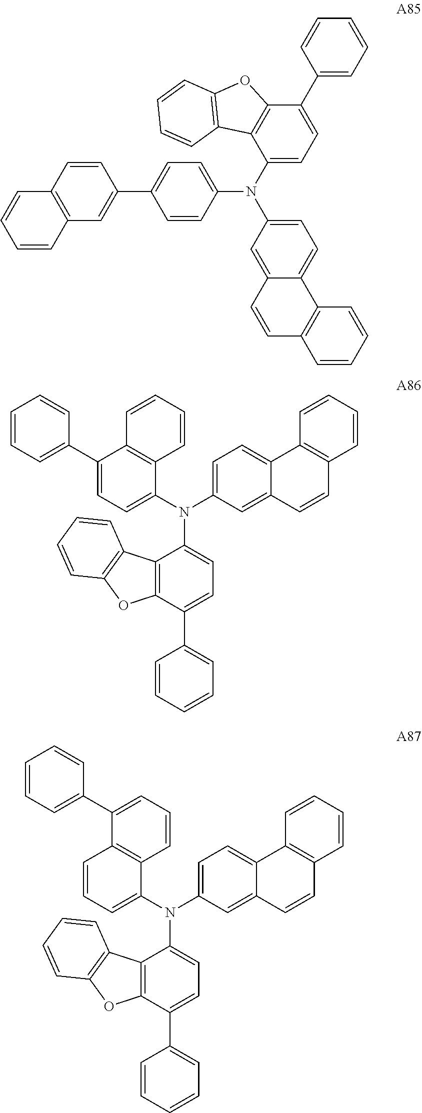 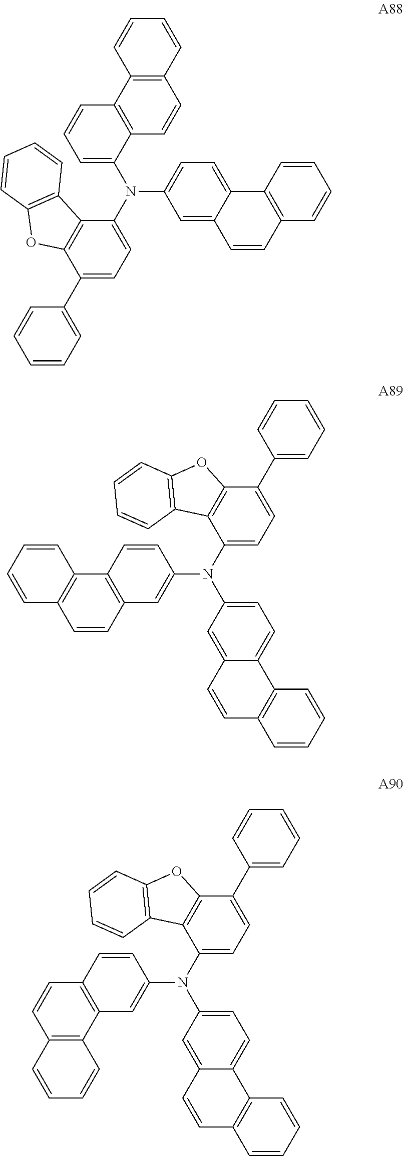 [SMILES: c1ccc(-c2ccc(N(c3ccc4c(ccc5ccccc54)c3)c3cccc4c3ccc3ccccc34)c3c2oc2ccccc23)cc1 ; c1ccc(-c2ccc(N(c3ccc4c(ccc5ccccc54)c3)c3ccc4c(ccc5ccccc54)c3)c3c2oc2ccccc23)cc1 ; c1ccc(-c2ccc(N(c3ccc4c(ccc5ccccc54)c3)c3ccc4ccc5ccccc5c4c3)c3c2oc2ccccc23)cc1] 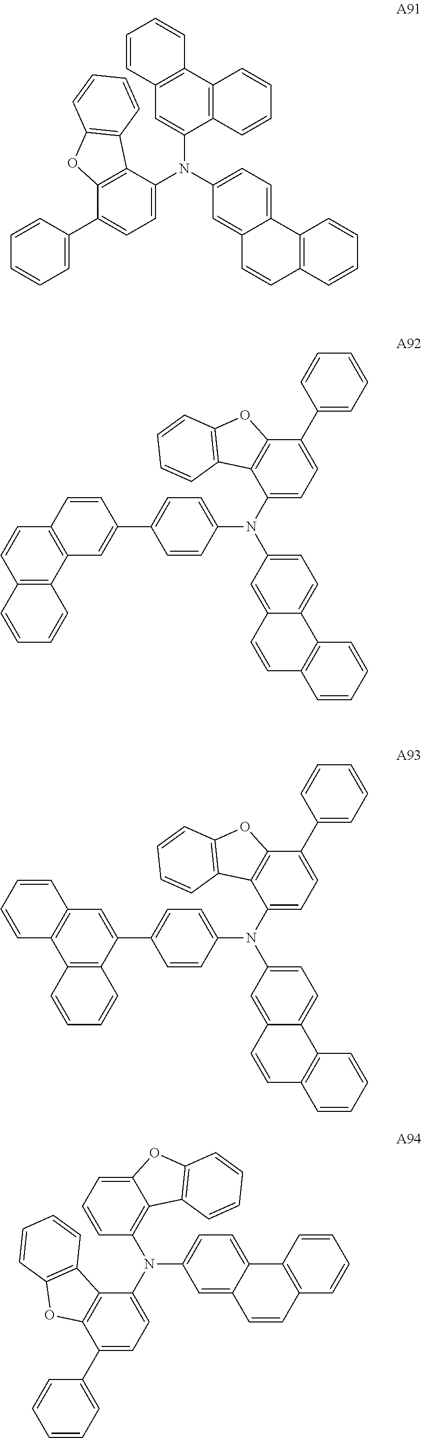 [SMILES: c1ccc(-c2ccc(N(c3ccc4c(ccc5ccccc54)c3)c3cc4ccccc4c4ccccc34)c3c2oc2ccccc23)cc1 ; c1ccc(-c2ccc(N(c3ccc(-c4ccc5ccc6ccccc6c5c4)cc3)c3ccc4c(ccc5ccccc54)c3)c3c2oc2ccccc23)cc1 ; c1ccc(-c2ccc(N(c3ccc(-c4cc5ccccc5c5ccccc45)cc3)c3ccc4c(ccc5ccccc54)c3)c3c2oc2ccccc23)cc1 ; c1ccc(-c2ccc(N(c3ccc4c(ccc5ccccc54)c3)c3cccc4oc5ccccc5c34)c3c2oc2ccccc23)cc1] 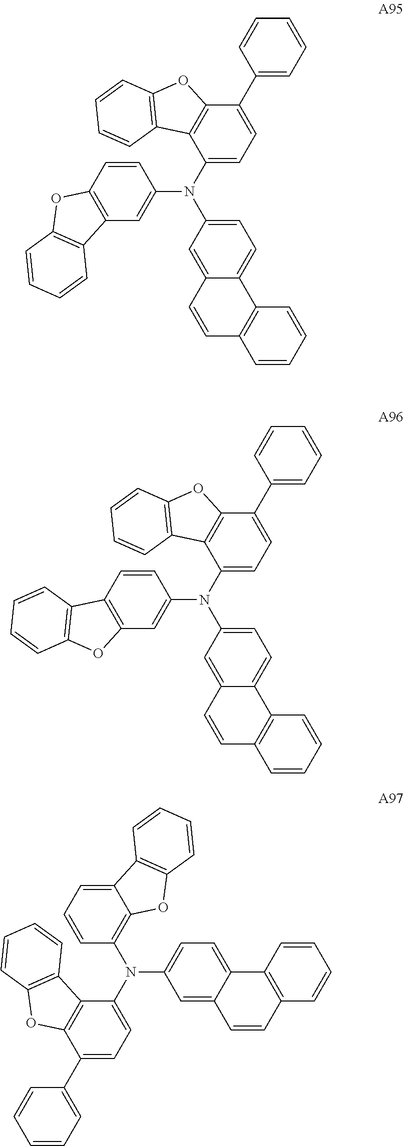 [SMILES: c1ccc(-c2ccc(N(c3ccc4c(ccc5ccccc54)c3)c3ccc4oc5ccccc5c4c3)c3c2oc2ccccc23)cc1 ; c1ccc(-c2ccc(N(c3ccc4c(ccc5ccccc54)c3)c3ccc4c(c3)oc3ccccc34)c3c2oc2ccccc23)cc1 ; c1ccc(-c2ccc(N(c3ccc4c(ccc5ccccc54)c3)c3cccc4c3oc3ccccc34)c3c2oc2ccccc23)cc1] 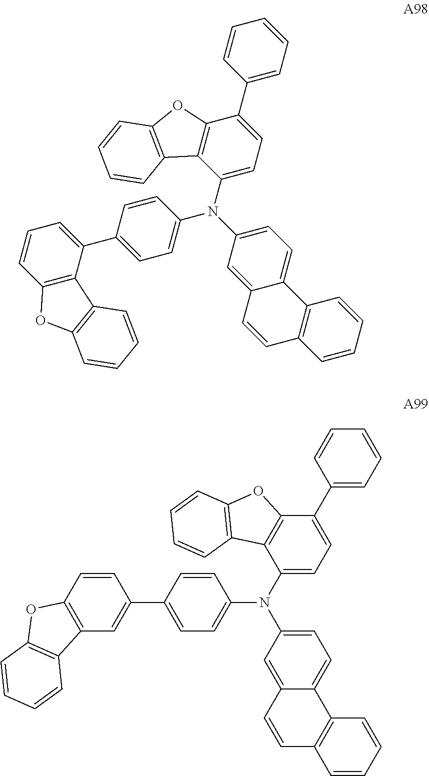 [SMILES: c1ccc(-c2ccc(N(c3ccc(-c4cccc5oc6ccccc6c45)cc3)c3ccc4c(ccc5ccccc54)c3)c3c2oc2ccccc23)cc1 ; c1ccc(-c2ccc(N(c3ccc(-c4ccc5oc6ccccc6c5c4)cc3)c3ccc4c(ccc5ccccc54)c3)c3c2oc2ccccc23)cc1] 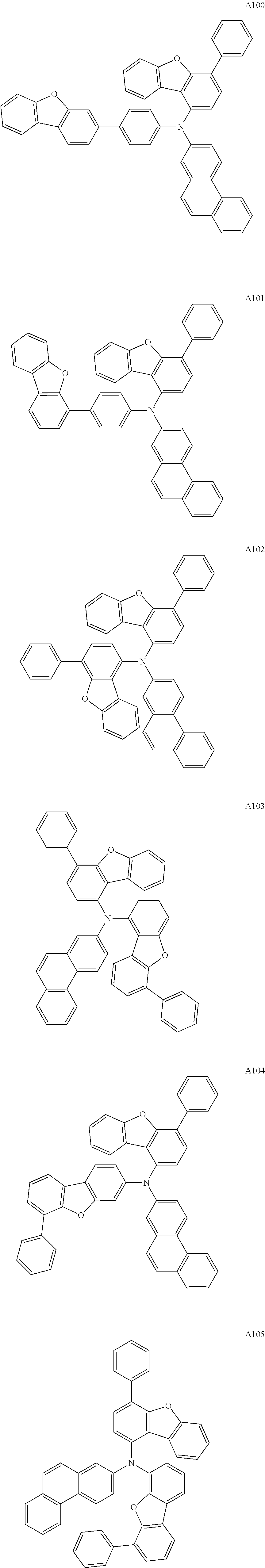 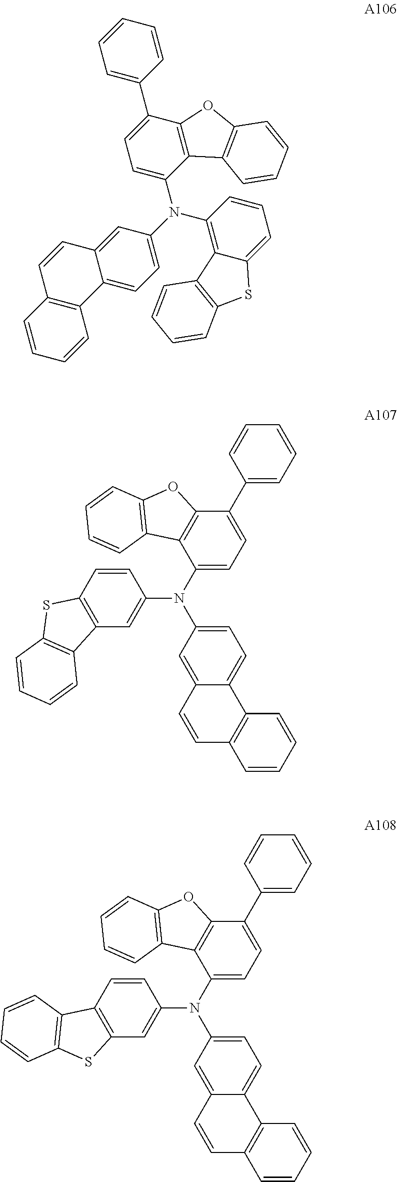 [SMILES: c1ccc(-c2ccc(N(c3ccc4c(ccc5ccccc54)c3)c3cccc4sc5ccccc5c34)c3c2oc2ccccc23)cc1 ; c1ccc(-c2ccc(N(c3ccc4c(ccc5ccccc54)c3)c3ccc4sc5ccccc5c4c3)c3c2oc2ccccc23)cc1 ; c1ccc(-c2ccc(N(c3ccc4c(ccc5ccccc54)c3)c3ccc4c(c3)sc3ccccc34)c3c2oc2ccccc23)cc1] 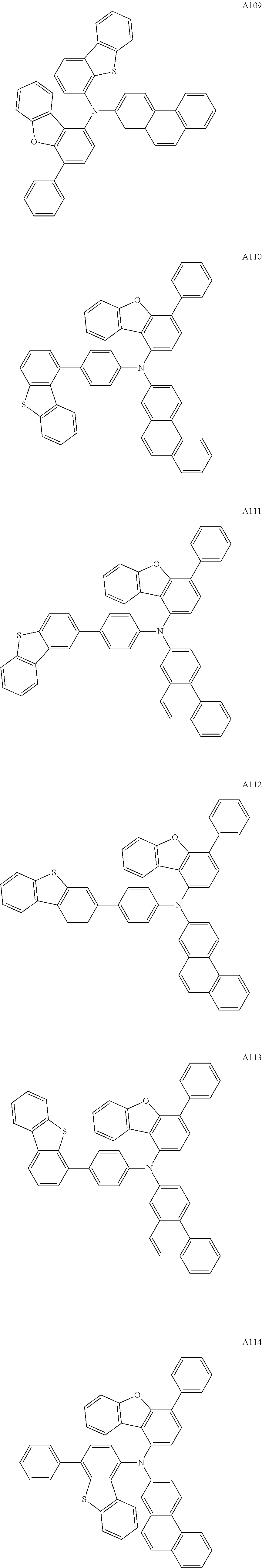 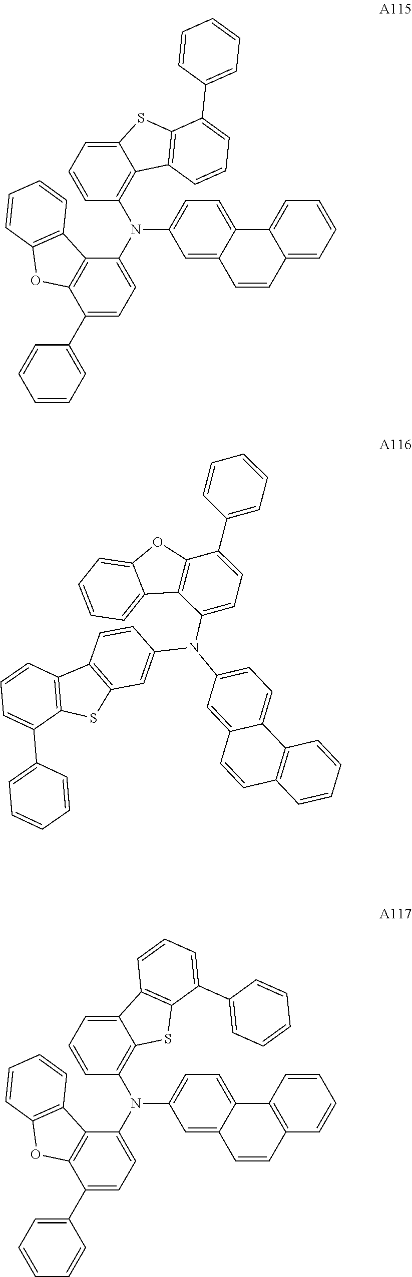 [SMILES: c1ccc(-c2ccc(N(c3ccc4c(ccc5ccccc54)c3)c3cccc4sc5c(-c6ccccc6)cccc5c34)c3c2oc2ccccc23)cc1 ; c1ccc(-c2ccc(N(c3ccc4c(ccc5ccccc54)c3)c3ccc4c(c3)sc3c(-c5ccccc5)cccc34)c3c2oc2ccccc23)cc1 ; c1ccc(-c2ccc(N(c3ccc4c(ccc5ccccc54)c3)c3cccc4c3sc3c(-c5ccccc5)cccc34)c3c2oc2ccccc23)cc1] 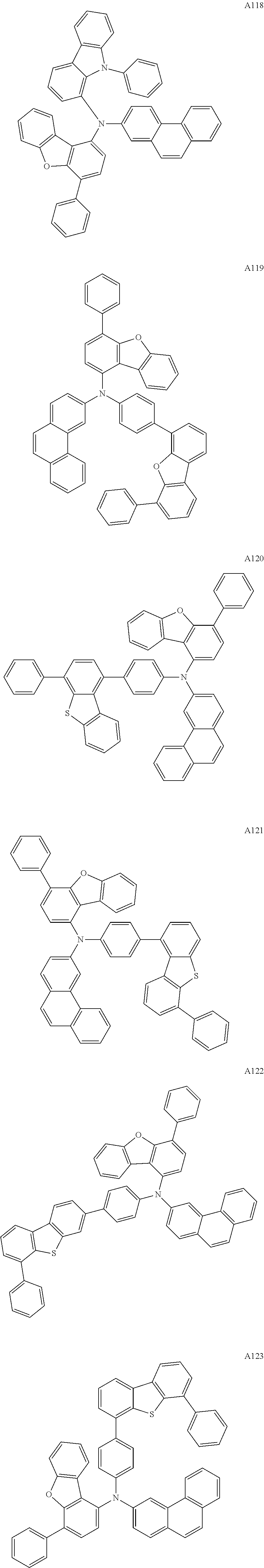 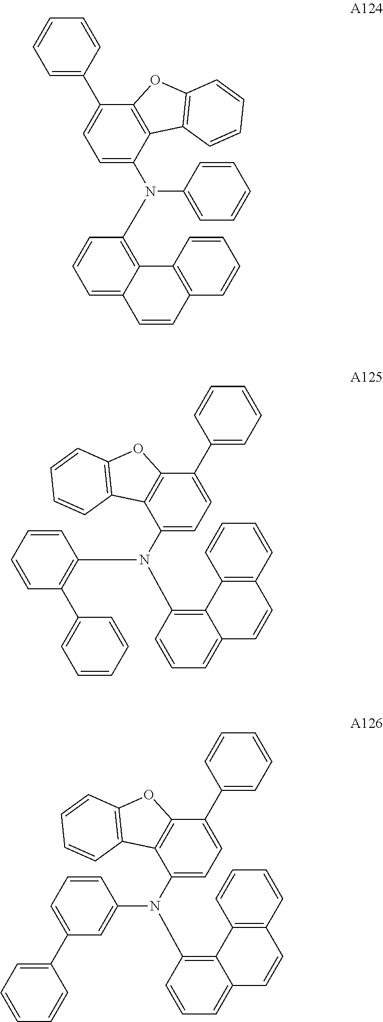 [SMILES: c1ccc(-c2ccc(N(c3ccccc3)c3cccc4ccc5ccccc5c34)c3c2oc2ccccc23)cc1 ; c1ccc(-c2ccccc2N(c2cccc3ccc4ccccc4c23)c2ccc(-c3ccccc3)c3oc4ccccc4c23)cc1 ; c1ccc(-c2cccc(N(c3cccc4ccc5ccccc5c34)c3ccc(-c4ccccc4)c4oc5ccccc5c34)c2)cc1] 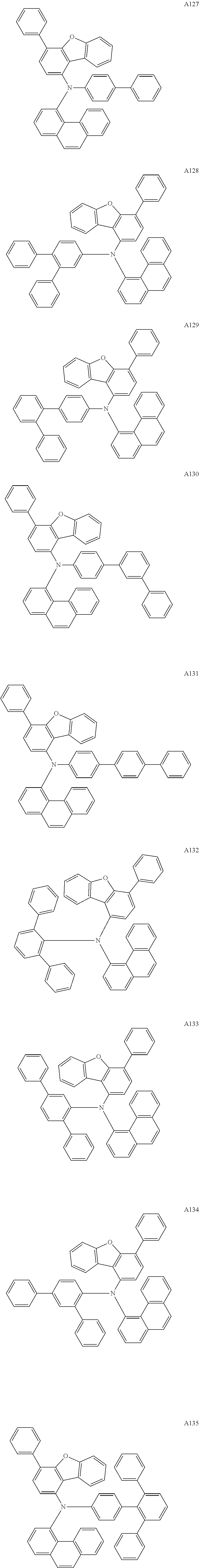 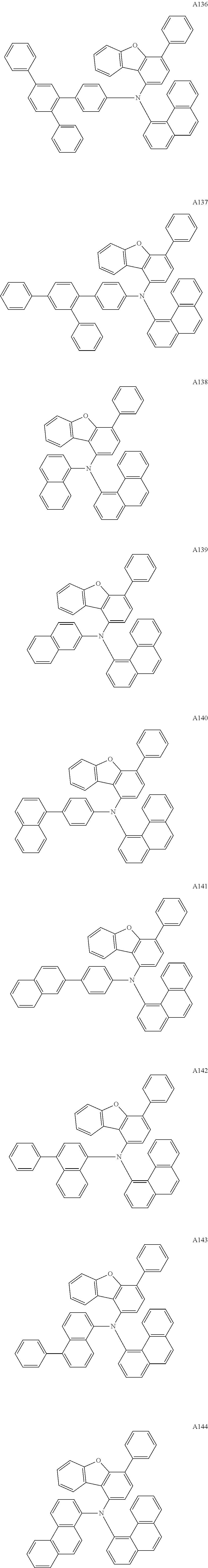 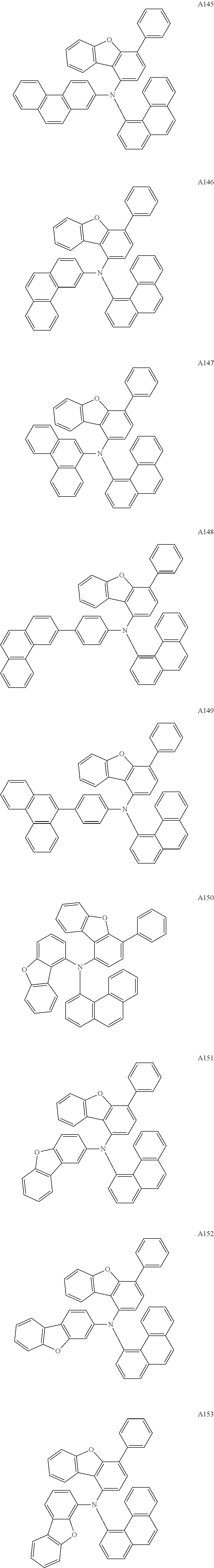 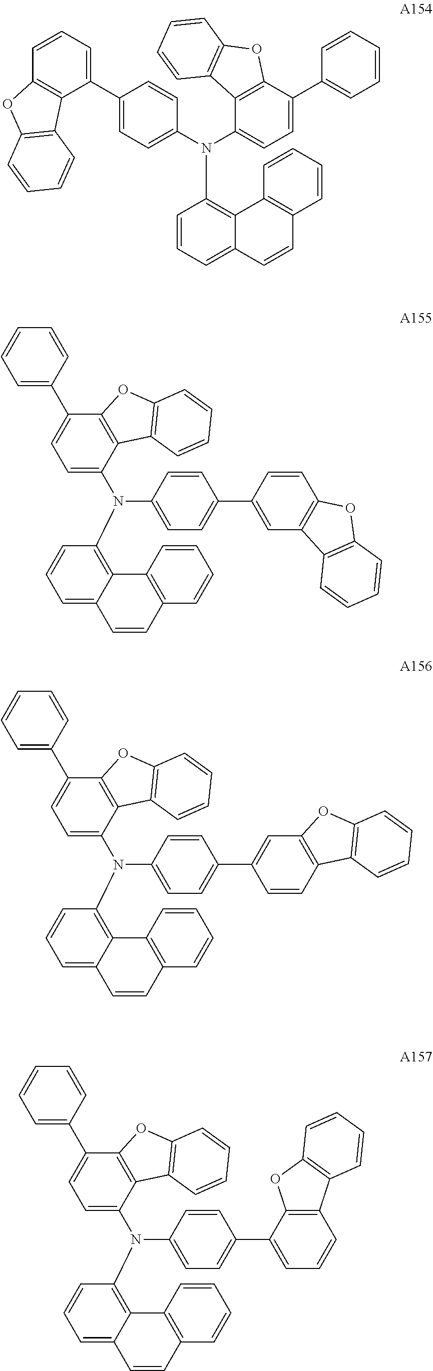 [SMILES: c1ccc(-c2ccc(N(c3ccc(-c4cccc5oc6ccccc6c45)cc3)c3cccc4ccc5ccccc5c34)c3c2oc2ccccc23)cc1 ; c1ccc(-c2ccc(N(c3ccc(-c4ccc5oc6ccccc6c5c4)cc3)c3cccc4ccc5ccccc5c34)c3c2oc2ccccc23)cc1 ; c1ccc(-c2ccc(N(c3ccc(-c4ccc5c(c4)oc4ccccc45)cc3)c3cccc4ccc5ccccc5c34)c3c2oc2ccccc23)cc1 ; c1ccc(-c2ccc(N(c3ccc(-c4cccc5c4oc4ccccc45)cc3)c3cccc4ccc5ccccc5c34)c3c2oc2ccccc23)cc1] 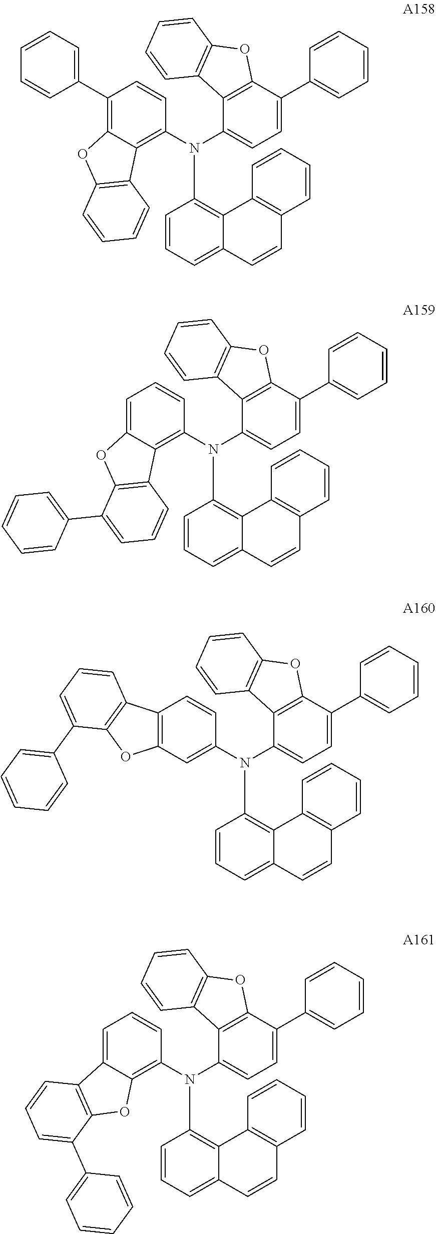 [SMILES: c1ccc(-c2ccc(N(c3cccc4ccc5ccccc5c34)c3ccc(-c4ccccc4)c4oc5ccccc5c34)c3c2oc2ccccc23)cc1 ; c1ccc(-c2cccc3c2oc2cccc(N(c4cccc5ccc6ccccc6c45)c4ccc(-c5ccccc5)c5oc6ccccc6c45)c23)cc1 ; c1ccc(-c2cccc3c2oc2cc(N(c4cccc5ccc6ccccc6c45)c4ccc(-c5ccccc5)c5oc6ccccc6c45)ccc23)cc1 ; c1ccc(-c2cccc3c2oc2c(N(c4cccc5ccc6ccccc6c45)c4ccc(-c5ccccc5)c5oc6ccccc6c45)cccc23)cc1] 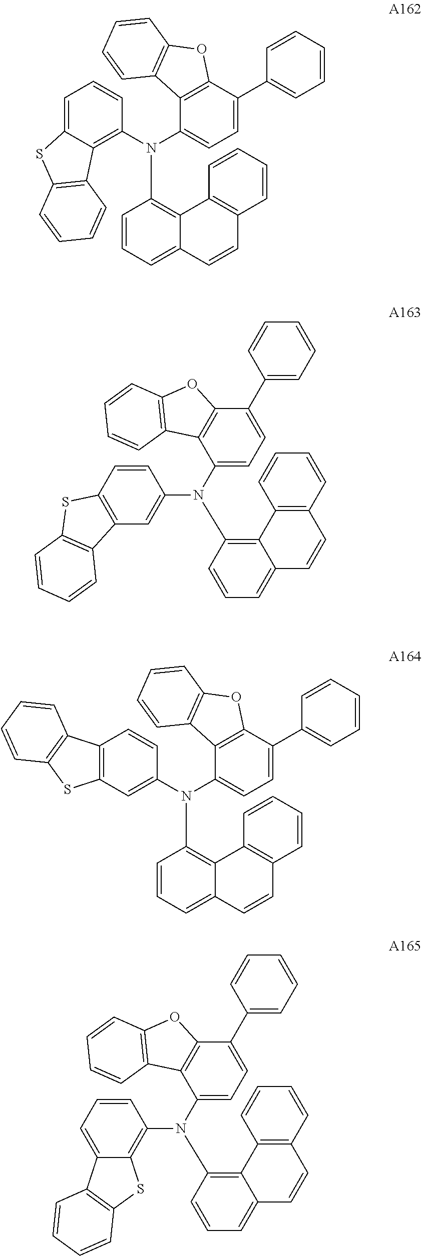 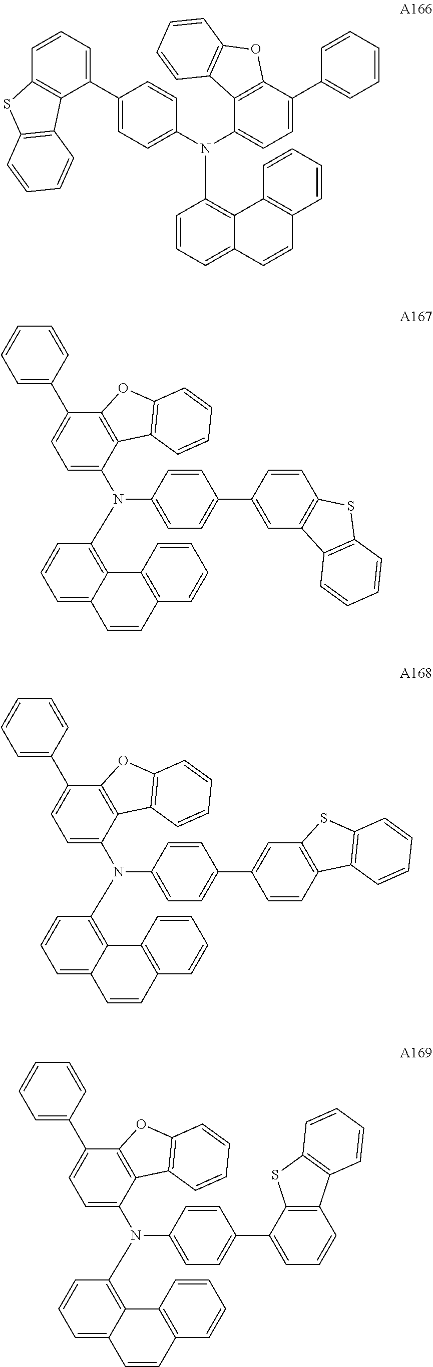 [SMILES: c1ccc(-c2ccc(N(c3ccc(-c4cccc5sc6ccccc6c45)cc3)c3cccc4ccc5ccccc5c34)c3c2oc2ccccc23)cc1 ; c1ccc(-c2ccc(N(c3ccc(-c4ccc5sc6ccccc6c5c4)cc3)c3cccc4ccc5ccccc5c34)c3c2oc2ccccc23)cc1 ; c1ccc(-c2ccc(N(c3ccc(-c4ccc5c(c4)sc4ccccc45)cc3)c3cccc4ccc5ccccc5c34)c3c2oc2ccccc23)cc1 ; c1ccc(-c2ccc(N(c3ccc(-c4cccc5c4sc4ccccc45)cc3)c3cccc4ccc5ccccc5c34)c3c2oc2ccccc23)cc1] 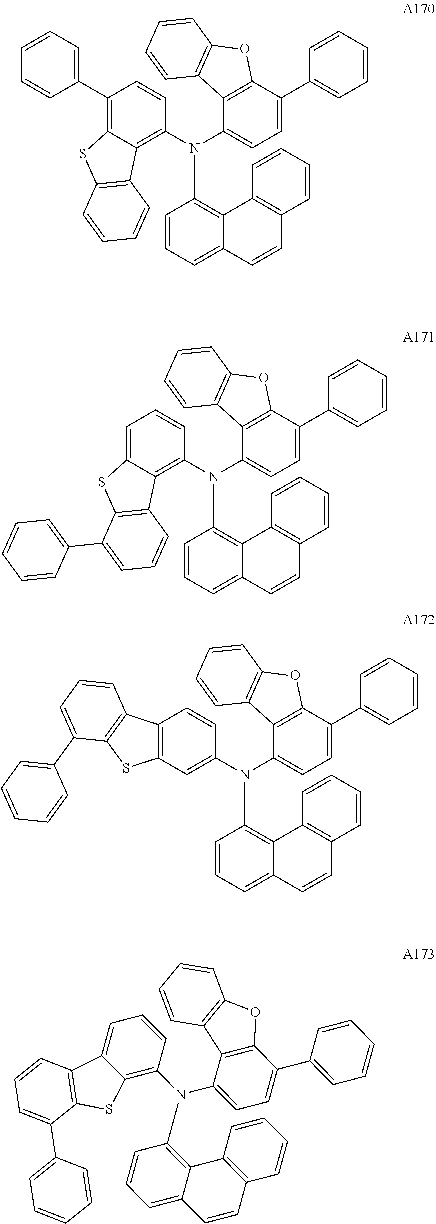 [SMILES: c1ccc(-c2ccc(N(c3cccc4ccc5ccccc5c34)c3ccc(-c4ccccc4)c4sc5ccccc5c34)c3c2oc2ccccc23)cc1 ; c1ccc(-c2ccc(N(c3cccc4ccc5ccccc5c34)c3cccc4sc5c(-c6ccccc6)cccc5c34)c3c2oc2ccccc23)cc1 ; c1ccc(-c2ccc(N(c3ccc4c(c3)sc3c(-c5ccccc5)cccc34)c3cccc4ccc5ccccc5c34)c3c2oc2ccccc23)cc1 ; c1ccc(-c2ccc(N(c3cccc4c3sc3c(-c5ccccc5)cccc34)c3cccc4ccc5ccccc5c34)c3c2oc2ccccc23)cc1] 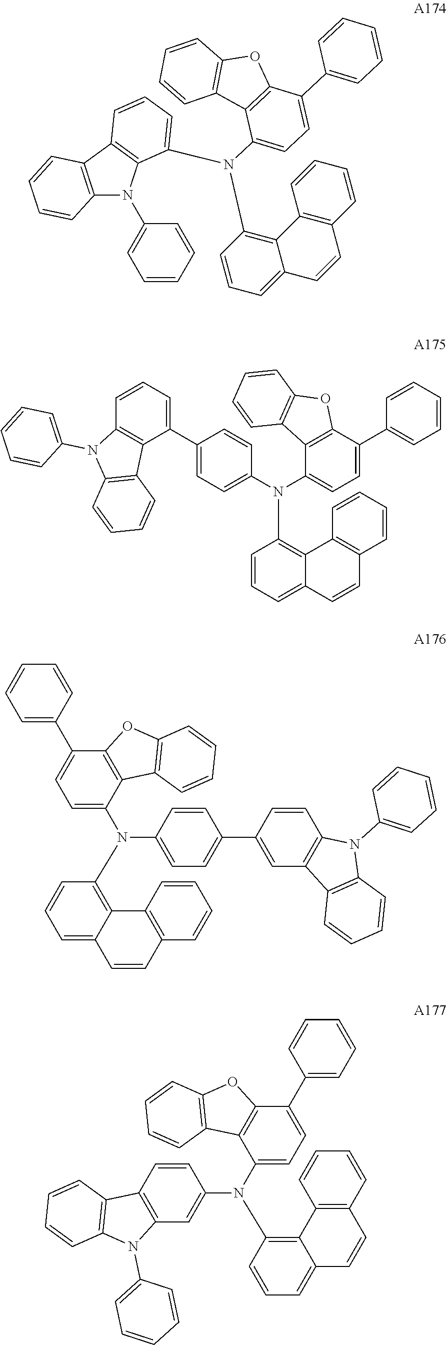 [SMILES: c1ccc(-c2ccc(N(c3cccc4ccc5ccccc5c34)c3cccc4c5ccccc5n(-c5ccccc5)c34)c3c2oc2ccccc23)cc1 ; c1ccc(-c2ccc(N(c3ccc(-c4cccc5c4c4ccccc4n5-c4ccccc4)cc3)c3cccc4ccc5ccccc5c34)c3c2oc2ccccc23)cc1 ; c1ccc(-c2ccc(N(c3ccc(-c4ccc5c(c4)c4ccccc4n5-c4ccccc4)cc3)c3cccc4ccc5ccccc5c34)c3c2oc2ccccc23)cc1 ; c1ccc(-c2ccc(N(c3ccc4c5ccccc5n(-c5ccccc5)c4c3)c3cccc4ccc5ccccc5c34)c3c2oc2ccccc23)cc1] 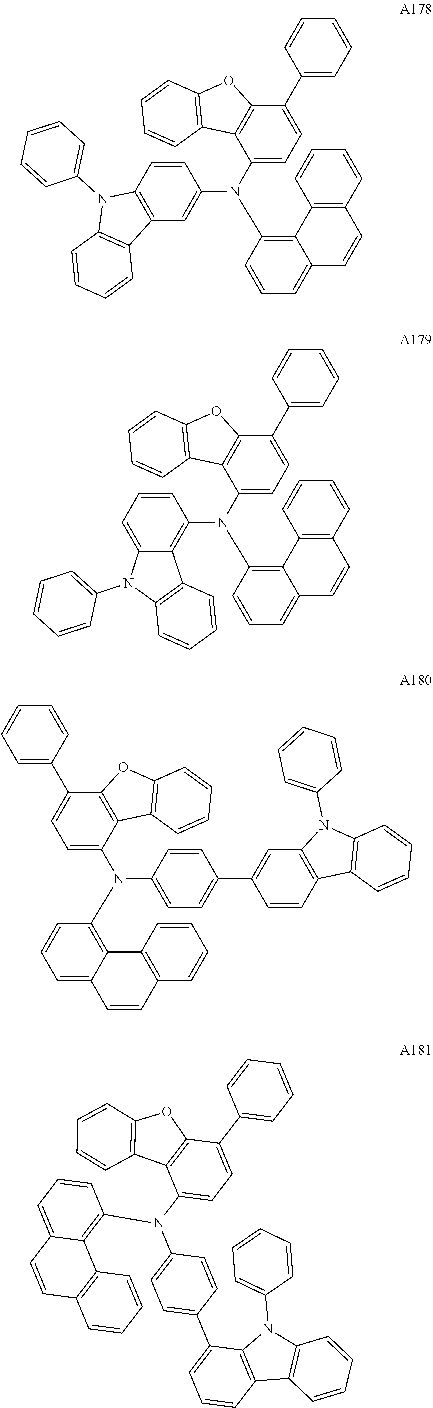 [SMILES: c1ccc(-c2ccc(N(c3ccc4c(c3)c3ccccc3n4-c3ccccc3)c3cccc4ccc5ccccc5c34)c3c2oc2ccccc23)cc1 ; c1ccc(-c2ccc(N(c3cccc4ccc5ccccc5c34)c3cccc4c3c3ccccc3n4-c3ccccc3)c3c2oc2ccccc23)cc1 ; c1ccc(-c2ccc(N(c3ccc(-c4ccc5c6ccccc6n(-c6ccccc6)c5c4)cc3)c3cccc4ccc5ccccc5c34)c3c2oc2ccccc23)cc1 ; c1ccc(-c2ccc(N(c3ccc(-c4cccc5c6ccccc6n(-c6ccccc6)c45)cc3)c3cccc4ccc5ccccc5c34)c3c2oc2ccccc23)cc1] 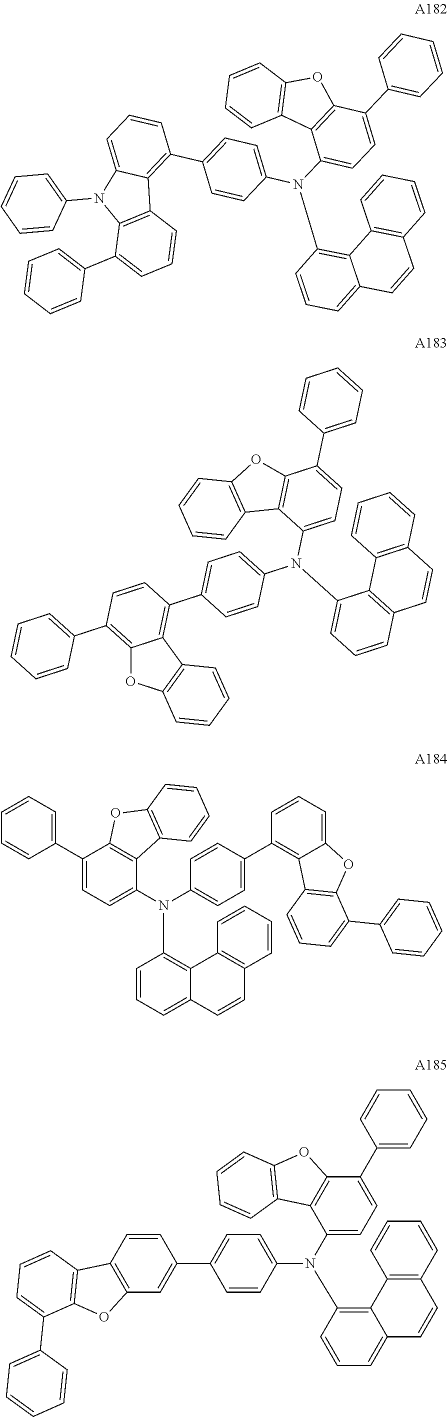 [SMILES: c1ccc(-c2ccc(N(c3ccc(-c4cccc5c4c4cccc(-c6ccccc6)c4n5-c4ccccc4)cc3)c3cccc4ccc5ccccc5c34)c3c2oc2ccccc23)cc1 ; c1ccc(-c2ccc(-c3ccc(N(c4cccc5ccc6ccccc6c45)c4ccc(-c5ccccc5)c5oc6ccccc6c45)cc3)c3c2oc2ccccc23)cc1 ; c1ccc(-c2cccc3c2oc2cccc(-c4ccc(N(c5cccc6ccc7ccccc7c56)c5ccc(-c6ccccc6)c6oc7ccccc7c56)cc4)c23)cc1 ; c1ccc(-c2cccc3c2oc2cc(-c4ccc(N(c5cccc6ccc7ccccc7c56)c5ccc(-c6ccccc6)c6oc7ccccc7c56)cc4)ccc23)cc1] 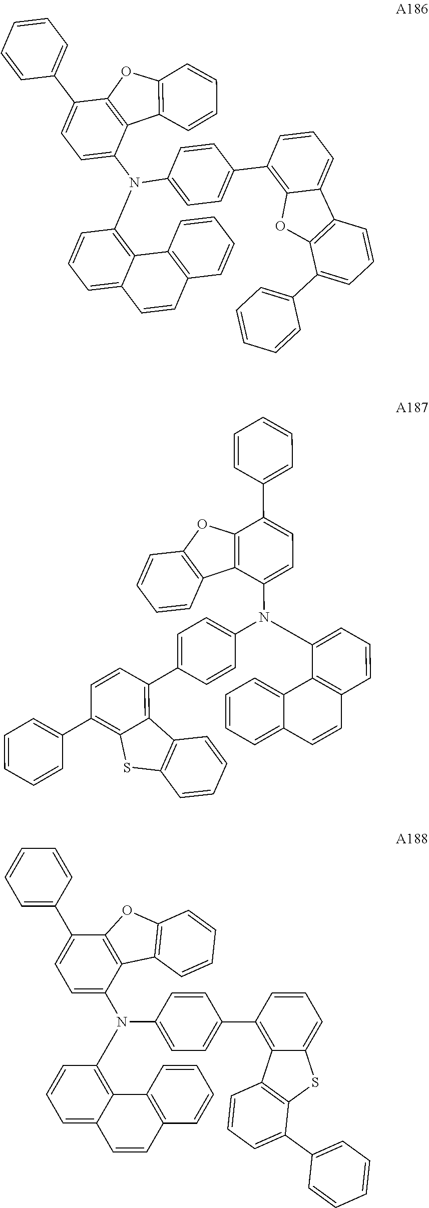 [SMILES: c1ccc(-c2cccc3c2oc2c(-c4ccc(N(c5cccc6ccc7ccccc7c56)c5ccc(-c6ccccc6)c6oc7ccccc7c56)cc4)cccc23)cc1 ; c1ccc(-c2ccc(N(c3ccc(-c4ccc(-c5ccccc5)c5sc6ccccc6c45)cc3)c3cccc4ccc5ccccc5c34)c3c2oc2ccccc23)cc1 ; c1ccc(-c2ccc(N(c3ccc(-c4cccc5sc6c(-c7ccccc7)cccc6c45)cc3)c3cccc4ccc5ccccc5c34)c3c2oc2ccccc23)cc1] 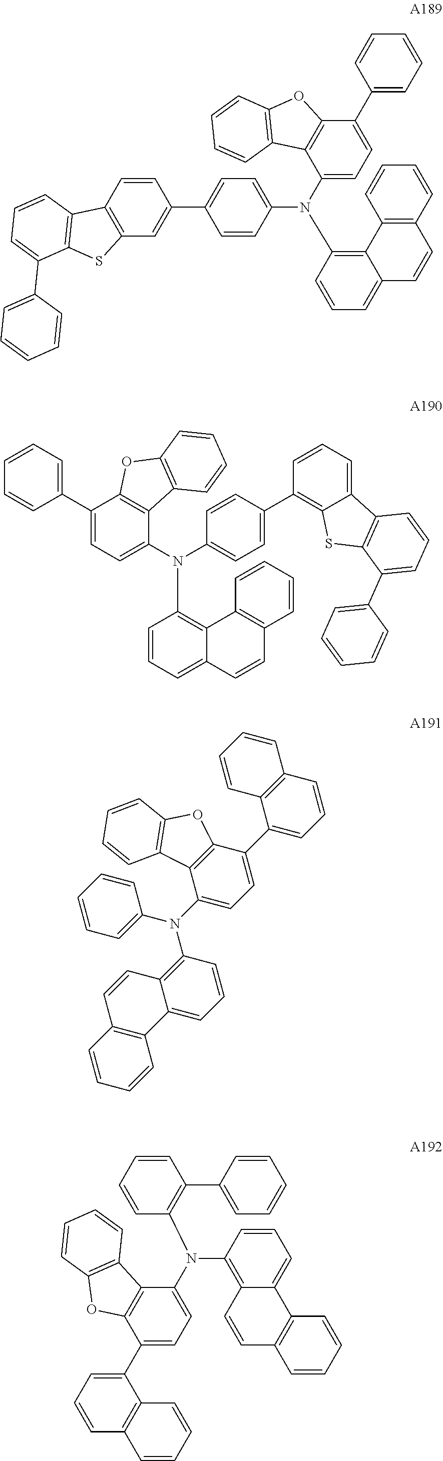 [SMILES: c1ccc(-c2ccc(N(c3ccc(-c4ccc5c(c4)sc4c(-c6ccccc6)cccc45)cc3)c3cccc4ccc5ccccc5c34)c3c2oc2ccccc23)cc1 ; c1ccc(-c2ccc(N(c3ccc(-c4cccc5c4sc4c(-c6ccccc6)cccc45)cc3)c3cccc4ccc5ccccc5c34)c3c2oc2ccccc23)cc1 ; c1ccc(N(c2cccc3c2ccc2ccccc23)c2ccc(-c3cccc4ccccc34)c3oc4ccccc4c23)cc1 ; c1ccc(-c2ccccc2N(c2cccc3c2ccc2ccccc23)c2ccc(-c3cccc4ccccc34)c3oc4ccccc4c23)cc1] 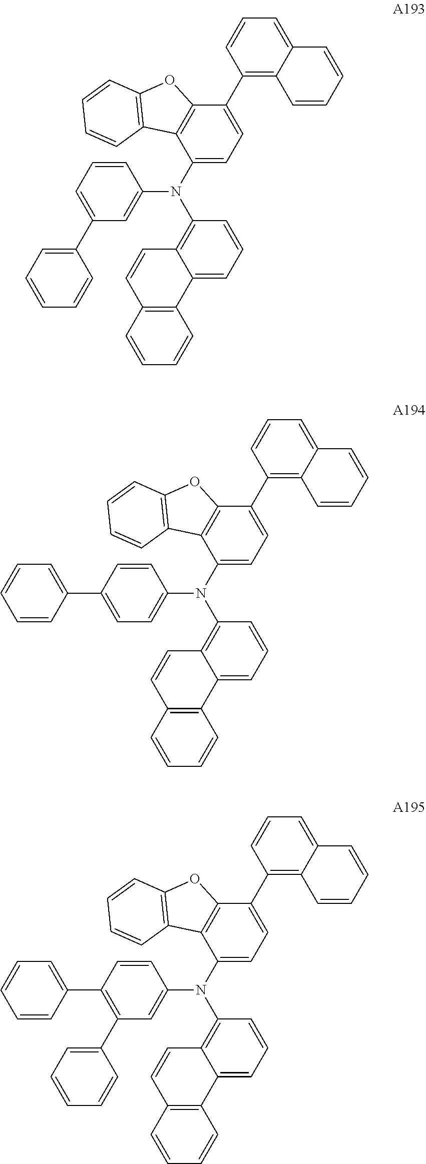 [SMILES: c1ccc(-c2cccc(N(c3cccc4c3ccc3ccccc34)c3ccc(-c4cccc5ccccc45)c4oc5ccccc5c34)c2)cc1 ; c1ccc(-c2ccc(N(c3cccc4c3ccc3ccccc34)c3ccc(-c4cccc5ccccc45)c4oc5ccccc5c34)cc2)cc1 ; c1ccc(-c2ccc(N(c3cccc4c3ccc3ccccc34)c3ccc(-c4cccc5ccccc45)c4oc5ccccc5c34)cc2-c2ccccc2)cc1] 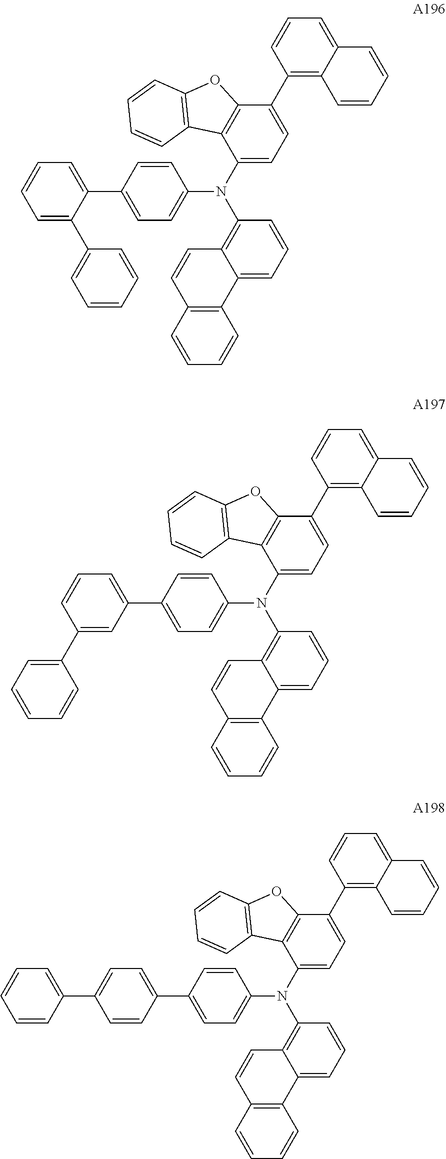 [SMILES: c1ccc(-c2ccccc2-c2ccc(N(c3cccc4c3ccc3ccccc34)c3ccc(-c4cccc5ccccc45)c4oc5ccccc5c34)cc2)cc1 ; c1ccc(-c2cccc(-c3ccc(N(c4cccc5c4ccc4ccccc45)c4ccc(-c5cccc6ccccc56)c5oc6ccccc6c45)cc3)c2)cc1 ; c1ccc(-c2ccc(-c3ccc(N(c4cccc5c4ccc4ccccc45)c4ccc(-c5cccc6ccccc56)c5oc6ccccc6c45)cc3)cc2)cc1] 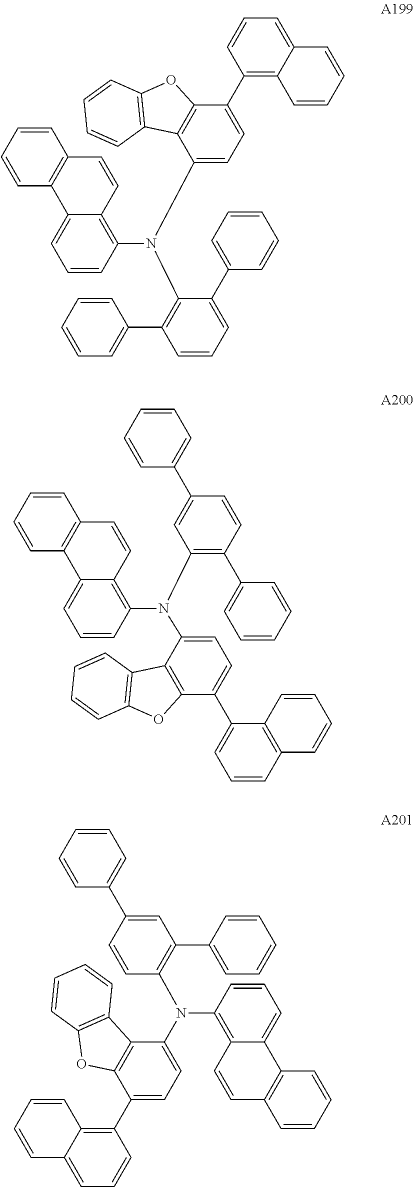 [SMILES: c1ccc(-c2cccc(-c3ccccc3)c2N(c2cccc3c2ccc2ccccc23)c2ccc(-c3cccc4ccccc34)c3oc4ccccc4c23)cc1 ; c1ccc(-c2ccc(-c3ccccc3)c(N(c3cccc4c3ccc3ccccc34)c3ccc(-c4cccc5ccccc45)c4oc5ccccc5c34)c2)cc1 ; c1ccc(-c2ccc(N(c3cccc4c3ccc3ccccc34)c3ccc(-c4cccc5ccccc45)c4oc5ccccc5c34)c(-c3ccccc3)c2)cc1] 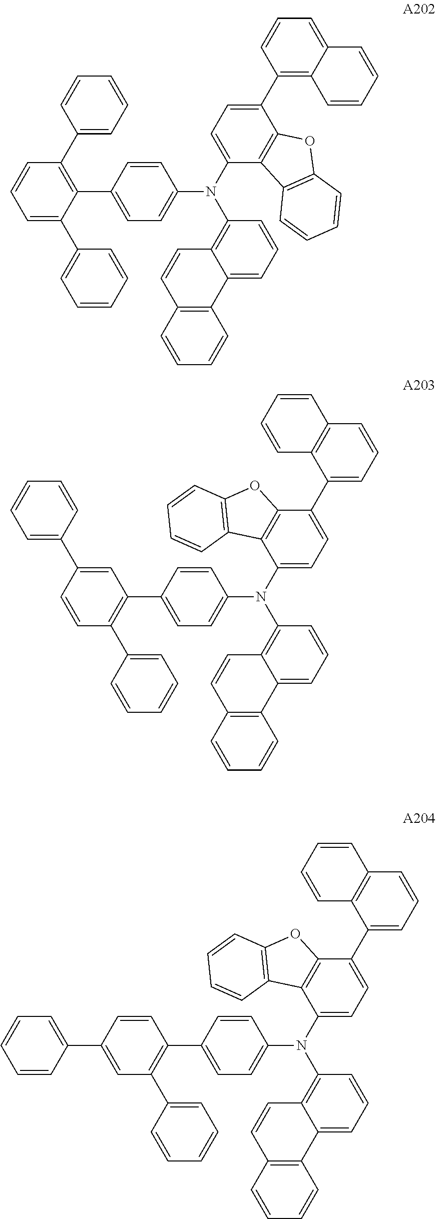 [SMILES: c1ccc(-c2cccc(-c3ccccc3)c2-c2ccc(N(c3cccc4c3ccc3ccccc34)c3ccc(-c4cccc5ccccc45)c4oc5ccccc5c34)cc2)cc1 ; c1ccc(-c2ccc(-c3ccccc3)c(-c3ccc(N(c4cccc5c4ccc4ccccc45)c4ccc(-c5cccc6ccccc56)c5oc6ccccc6c45)cc3)c2)cc1 ; c1ccc(-c2ccc(-c3ccc(N(c4cccc5c4ccc4ccccc45)c4ccc(-c5cccc6ccccc56)c5oc6ccccc6c45)cc3)c(-c3ccccc3)c2)cc1] 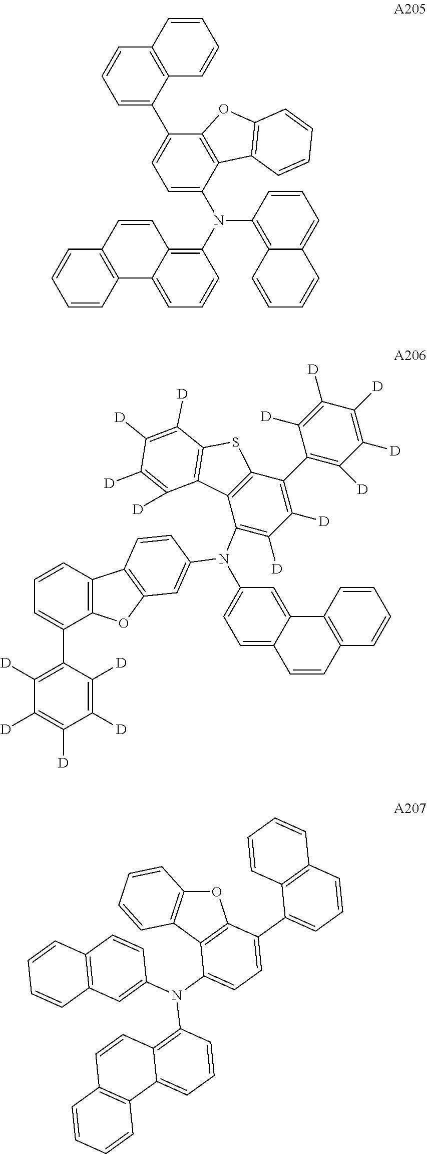 [SMILES: c1ccc2c(-c3ccc(N(c4cccc5ccccc45)c4cccc5c4ccc4ccccc45)c4c3oc3ccccc34)cccc2c1 ; [2H]c1c([2H])c([2H])c(-c2cccc3c2oc2cc(N(c4ccc5ccc6ccccc6c5c4)c4c([2H])c([2H])c(-c5c([2H])c([2H])c([2H])c([2H])c5[2H])c5sc6c([2H])c([2H])c([2H])c([2H])c6c45)ccc23)c([2H])c1[2H] ; c1ccc2cc(N(c3cccc4c3ccc3ccccc34)c3ccc(-c4cccc5ccccc45)c4oc5ccccc5c34)ccc2c1] 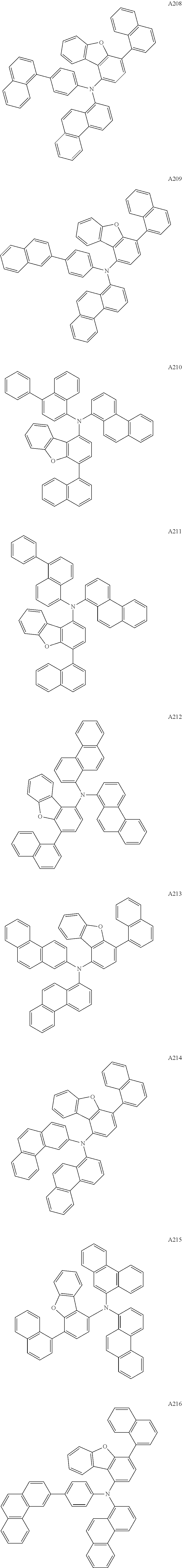 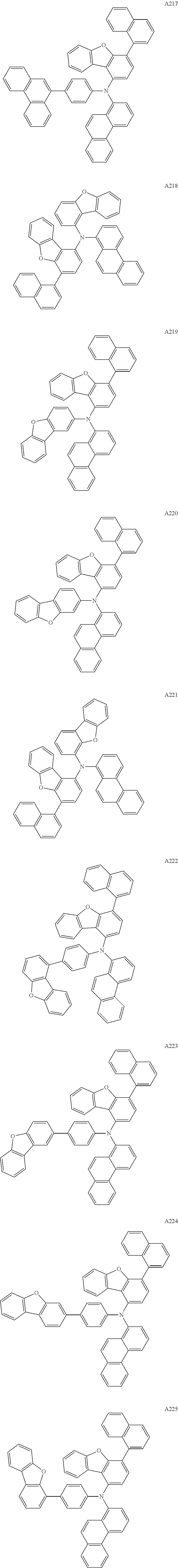 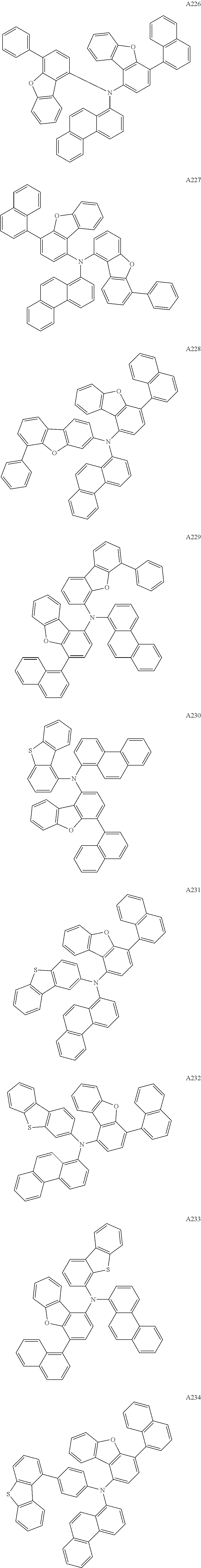 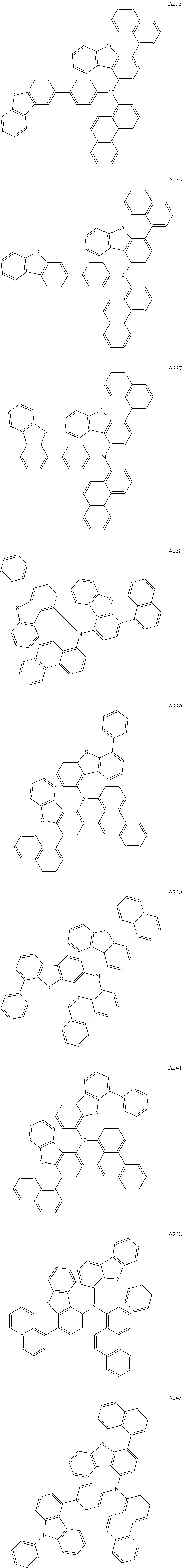 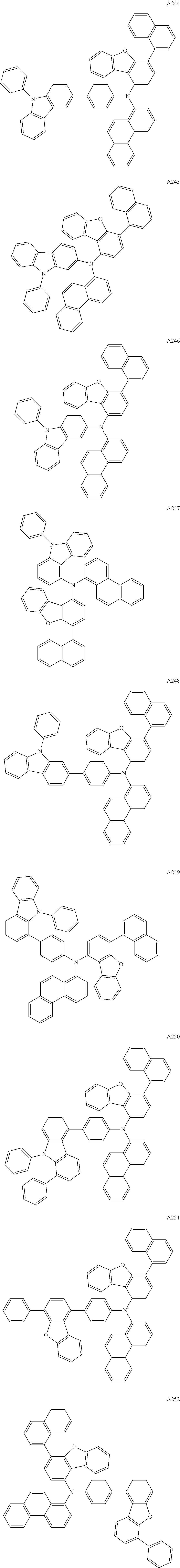 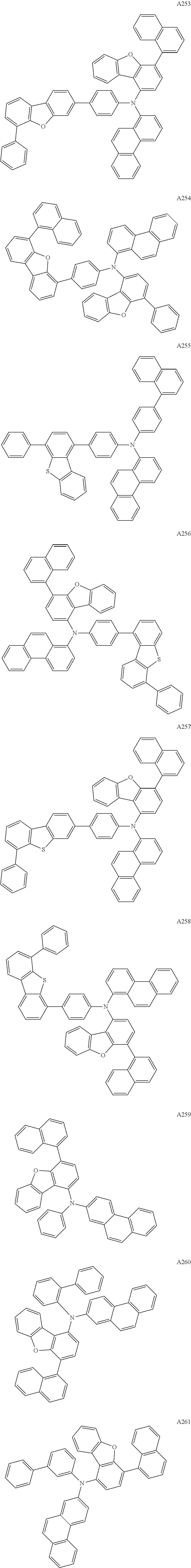 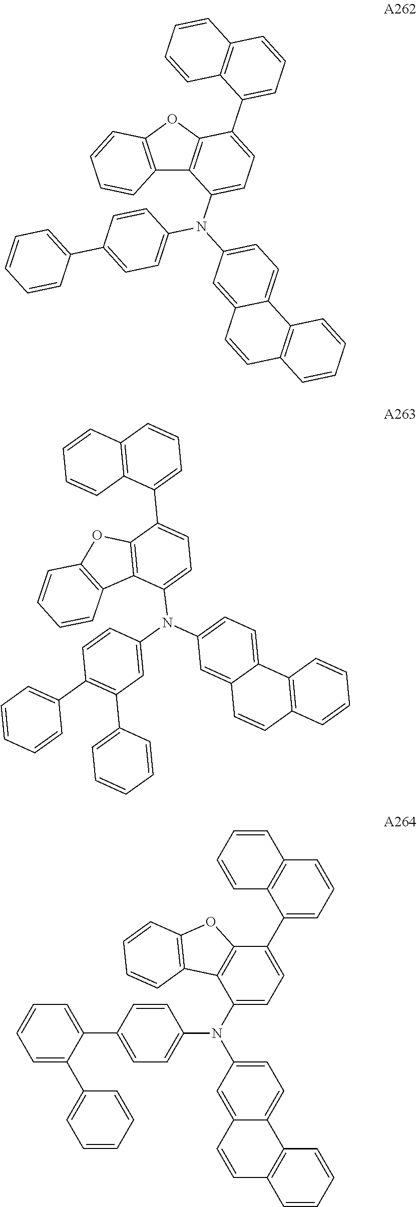 [SMILES: c1ccc(-c2ccc(N(c3ccc4c(ccc5ccccc54)c3)c3ccc(-c4cccc5ccccc45)c4oc5ccccc5c34)cc2)cc1 ; c1ccc(-c2ccc(N(c3ccc4c(ccc5ccccc54)c3)c3ccc(-c4cccc5ccccc45)c4oc5ccccc5c34)cc2-c2ccccc2)cc1 ; c1ccc(-c2ccccc2-c2ccc(N(c3ccc4c(ccc5ccccc54)c3)c3ccc(-c4cccc5ccccc45)c4oc5ccccc5c34)cc2)cc1] 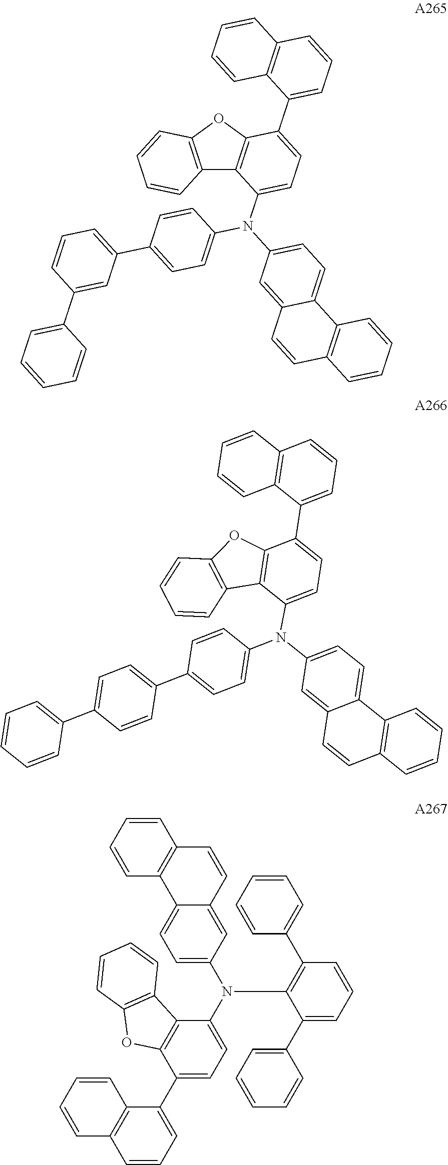 [SMILES: c1ccc(-c2cccc(-c3ccc(N(c4ccc5c(ccc6ccccc65)c4)c4ccc(-c5cccc6ccccc56)c5oc6ccccc6c45)cc3)c2)cc1 ; c1ccc(-c2ccc(-c3ccc(N(c4ccc5c(ccc6ccccc65)c4)c4ccc(-c5cccc6ccccc56)c5oc6ccccc6c45)cc3)cc2)cc1 ; c1ccc(-c2cccc(-c3ccccc3)c2N(c2ccc3c(ccc4ccccc43)c2)c2ccc(-c3cccc4ccccc34)c3oc4ccccc4c23)cc1] 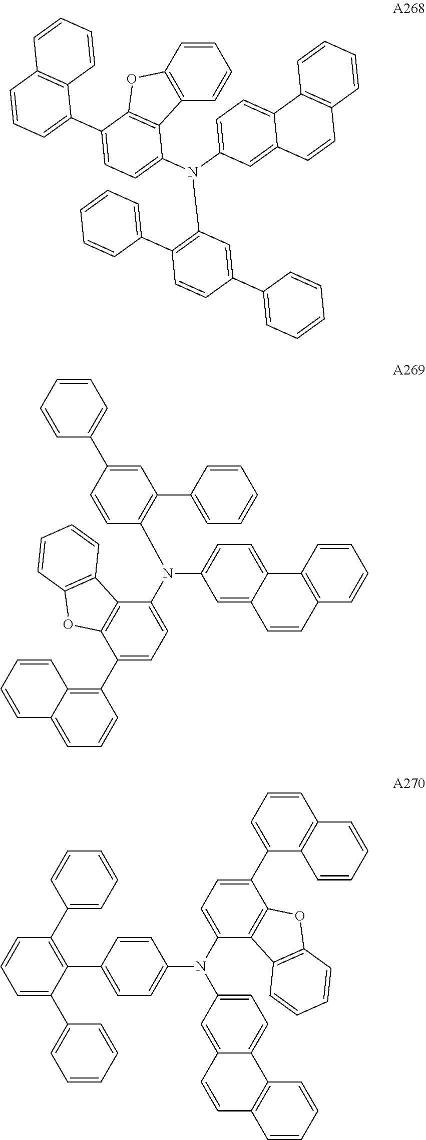 [SMILES: c1ccc(-c2ccc(-c3ccccc3)c(N(c3ccc4c(ccc5ccccc54)c3)c3ccc(-c4cccc5ccccc45)c4oc5ccccc5c34)c2)cc1 ; c1ccc(-c2ccc(N(c3ccc4c(ccc5ccccc54)c3)c3ccc(-c4cccc5ccccc45)c4oc5ccccc5c34)c(-c3ccccc3)c2)cc1 ; c1ccc(-c2cccc(-c3ccccc3)c2-c2ccc(N(c3ccc4c(ccc5ccccc54)c3)c3ccc(-c4cccc5ccccc45)c4oc5ccccc5c34)cc2)cc1] 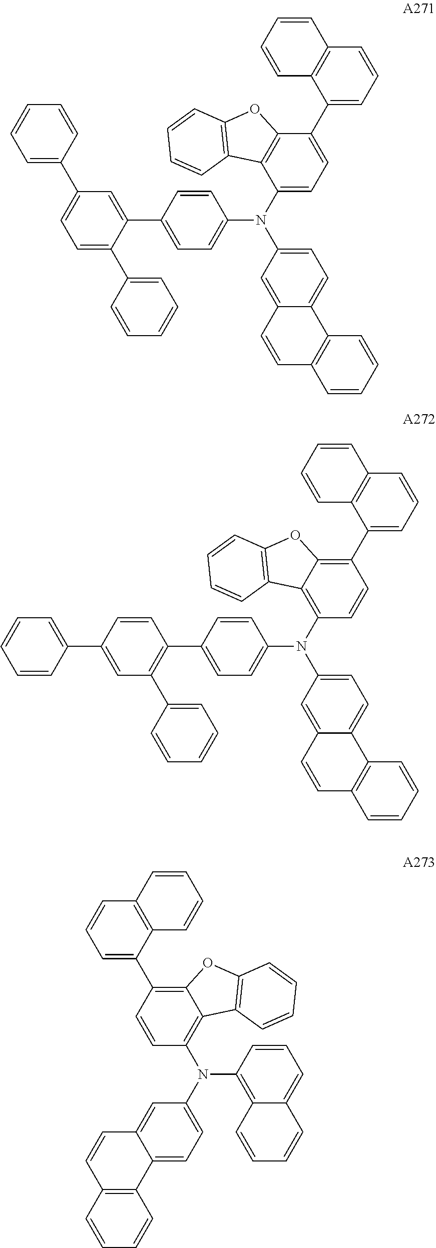 [SMILES: c1ccc(-c2ccc(-c3ccccc3)c(-c3ccc(N(c4ccc5c(ccc6ccccc65)c4)c4ccc(-c5cccc6ccccc56)c5oc6ccccc6c45)cc3)c2)cc1 ; c1ccc(-c2ccc(-c3ccc(N(c4ccc5c(ccc6ccccc65)c4)c4ccc(-c5cccc6ccccc56)c5oc6ccccc6c45)cc3)c(-c3ccccc3)c2)cc1 ; c1ccc2c(-c3ccc(N(c4ccc5c(ccc6ccccc65)c4)c4cccc5ccccc45)c4c3oc3ccccc34)cccc2c1] 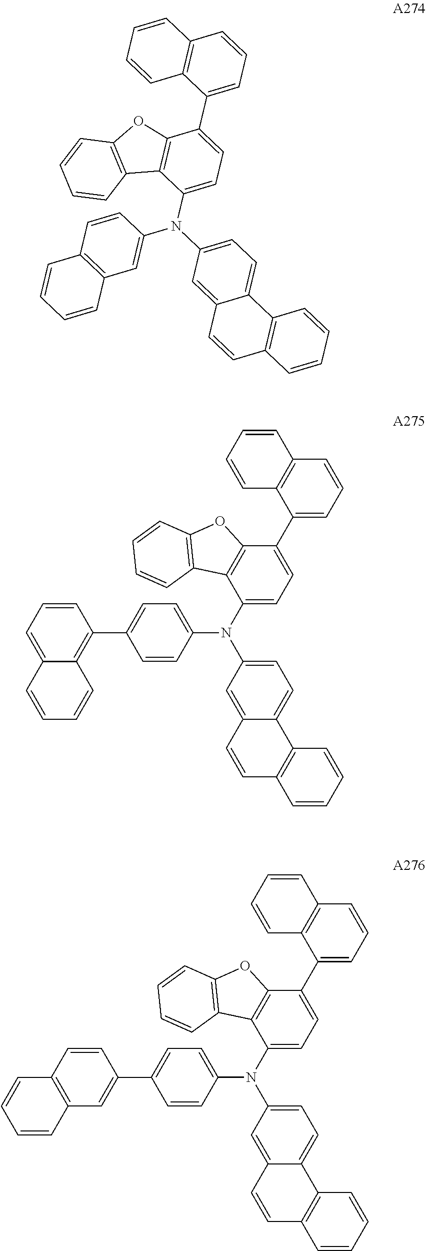 [SMILES: c1ccc2cc(N(c3ccc4c(ccc5ccccc54)c3)c3ccc(-c4cccc5ccccc45)c4oc5ccccc5c34)ccc2c1 ; c1ccc2c(-c3ccc(N(c4ccc5c(ccc6ccccc65)c4)c4ccc(-c5cccc6ccccc56)c5oc6ccccc6c45)cc3)cccc2c1 ; c1ccc2cc(-c3ccc(N(c4ccc5c(ccc6ccccc65)c4)c4ccc(-c5cccc6ccccc56)c5oc6ccccc6c45)cc3)ccc2c1] 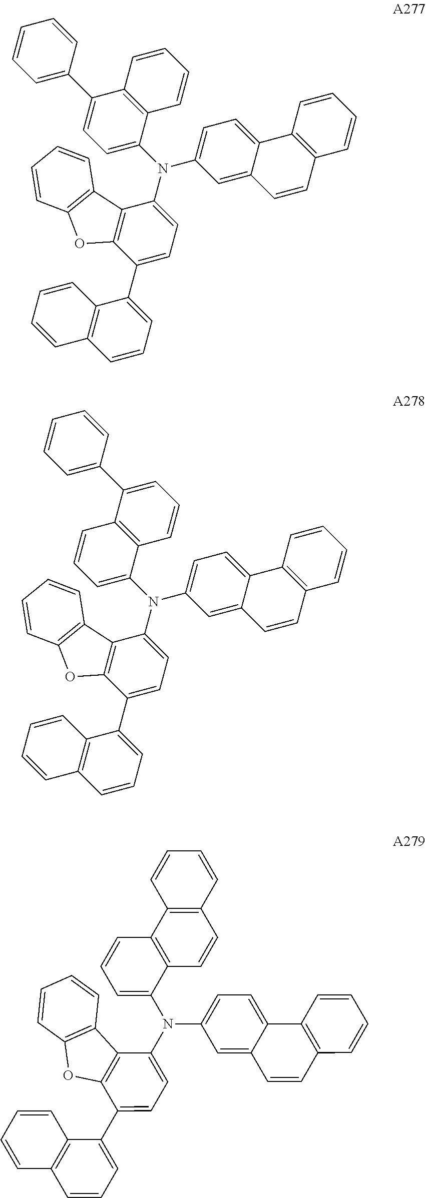 [SMILES: c1ccc(-c2ccc(N(c3ccc4c(ccc5ccccc54)c3)c3ccc(-c4cccc5ccccc45)c4oc5ccccc5c34)c3ccccc23)cc1 ; c1ccc(-c2cccc3c(N(c4ccc5c(ccc6ccccc65)c4)c4ccc(-c5cccc6ccccc56)c5oc6ccccc6c45)cccc23)cc1 ; c1ccc2c(-c3ccc(N(c4ccc5c(ccc6ccccc65)c4)c4cccc5c4ccc4ccccc45)c4c3oc3ccccc34)cccc2c1] 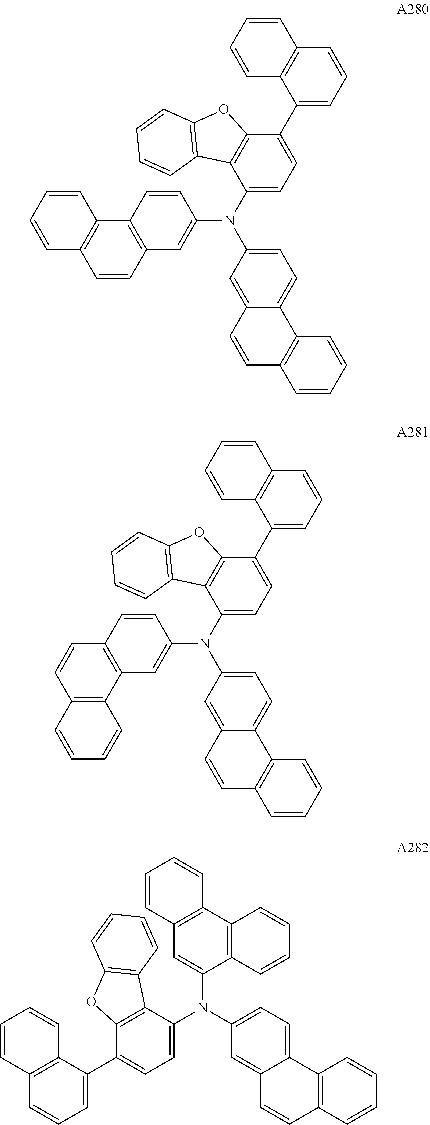 [SMILES: c1ccc2c(-c3ccc(N(c4ccc5c(ccc6ccccc65)c4)c4ccc5c(ccc6ccccc65)c4)c4c3oc3ccccc34)cccc2c1 ; c1ccc2c(-c3ccc(N(c4ccc5c(ccc6ccccc65)c4)c4ccc5ccc6ccccc6c5c4)c4c3oc3ccccc34)cccc2c1 ; c1ccc2c(-c3ccc(N(c4ccc5c(ccc6ccccc65)c4)c4cc5ccccc5c5ccccc45)c4c3oc3ccccc34)cccc2c1] 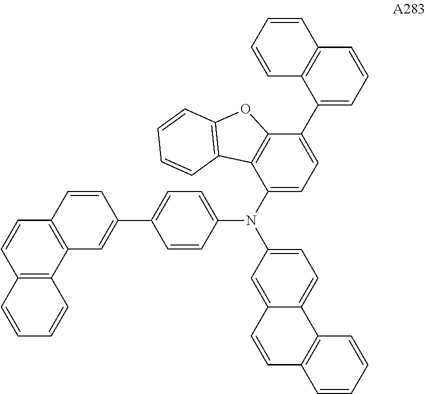 [SMILES: c1ccc2c(-c3ccc(N(c4ccc(-c5ccc6ccc7ccccc7c6c5)cc4)c4ccc5c(ccc6ccccc65)c4)c4c3oc3ccccc34)cccc2c1] 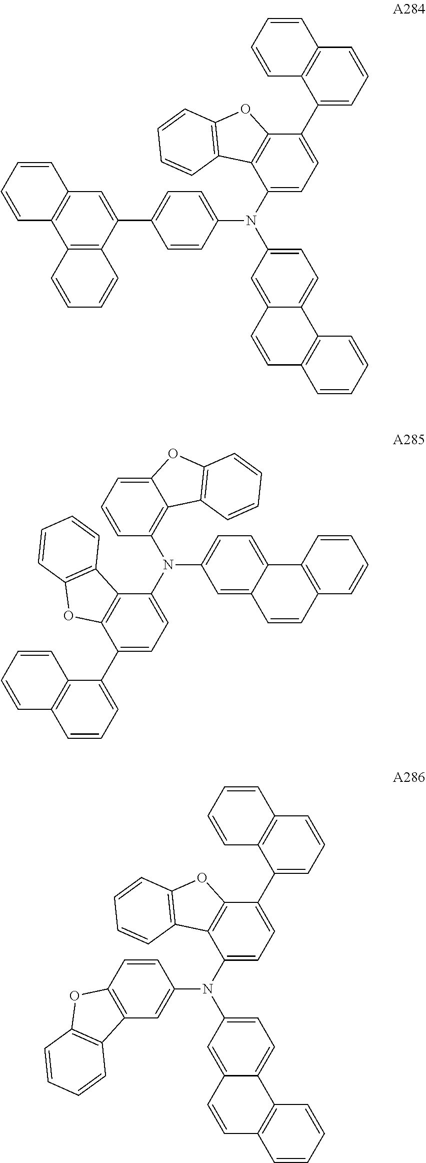 [SMILES: c1ccc2c(-c3ccc(N(c4ccc(-c5cc6ccccc6c6ccccc56)cc4)c4ccc5c(ccc6ccccc65)c4)c4c3oc3ccccc34)cccc2c1 ; c1ccc2c(-c3ccc(N(c4ccc5c(ccc6ccccc65)c4)c4cccc5oc6ccccc6c45)c4c3oc3ccccc34)cccc2c1 ; c1ccc2c(-c3ccc(N(c4ccc5c(ccc6ccccc65)c4)c4ccc5oc6ccccc6c5c4)c4c3oc3ccccc34)cccc2c1] 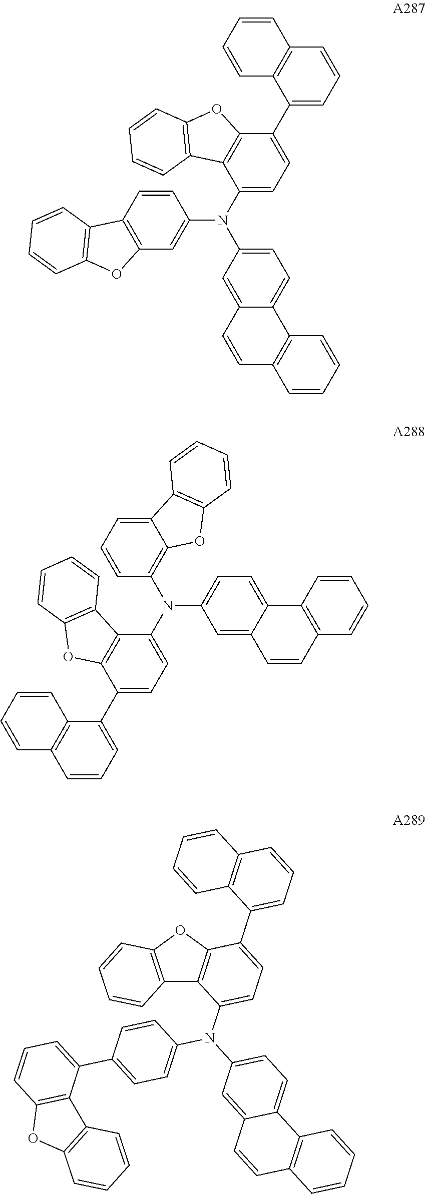 [SMILES: c1ccc2c(-c3ccc(N(c4ccc5c(ccc6ccccc65)c4)c4ccc5c(c4)oc4ccccc45)c4c3oc3ccccc34)cccc2c1 ; c1ccc2c(-c3ccc(N(c4ccc5c(ccc6ccccc65)c4)c4cccc5c4oc4ccccc45)c4c3oc3ccccc34)cccc2c1 ; c1ccc2c(-c3ccc(N(c4ccc(-c5cccc6oc7ccccc7c56)cc4)c4ccc5c(ccc6ccccc65)c4)c4c3oc3ccccc34)cccc2c1] 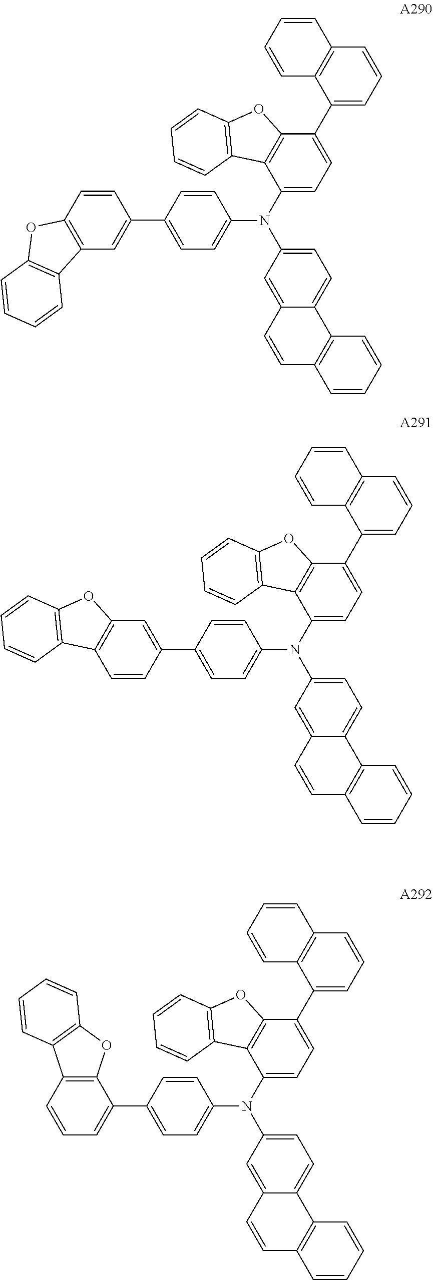 [SMILES: c1ccc2c(-c3ccc(N(c4ccc(-c5ccc6oc7ccccc7c6c5)cc4)c4ccc5c(ccc6ccccc65)c4)c4c3oc3ccccc34)cccc2c1 ; c1ccc2c(-c3ccc(N(c4ccc(-c5ccc6c(c5)oc5ccccc56)cc4)c4ccc5c(ccc6ccccc65)c4)c4c3oc3ccccc34)cccc2c1 ; c1ccc2c(-c3ccc(N(c4ccc(-c5cccc6c5oc5ccccc56)cc4)c4ccc5c(ccc6ccccc65)c4)c4c3oc3ccccc34)cccc2c1] 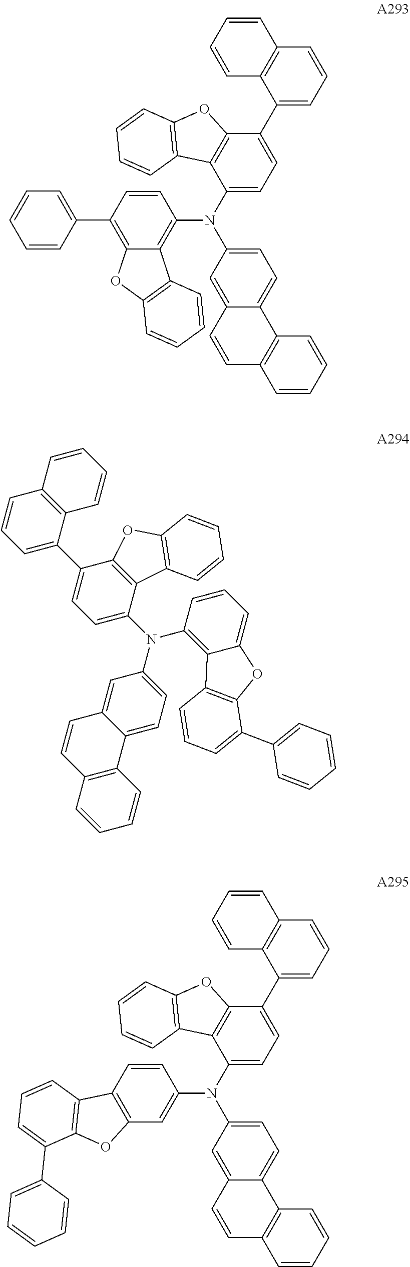 [SMILES: c1ccc(-c2ccc(N(c3ccc4c(ccc5ccccc54)c3)c3ccc(-c4cccc5ccccc45)c4oc5ccccc5c34)c3c2oc2ccccc23)cc1 ; c1ccc(-c2cccc3c2oc2cccc(N(c4ccc5c(ccc6ccccc65)c4)c4ccc(-c5cccc6ccccc56)c5oc6ccccc6c45)c23)cc1 ; c1ccc(-c2cccc3c2oc2cc(N(c4ccc5c(ccc6ccccc65)c4)c4ccc(-c5cccc6ccccc56)c5oc6ccccc6c45)ccc23)cc1] 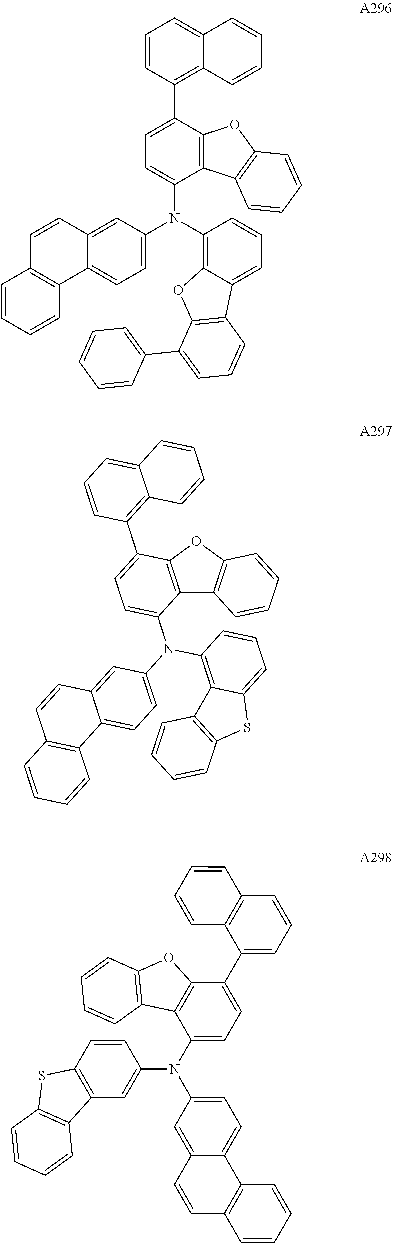 [SMILES: c1ccc(-c2cccc3c2oc2c(N(c4ccc5c(ccc6ccccc65)c4)c4ccc(-c5cccc6ccccc56)c5oc6ccccc6c45)cccc23)cc1 ; c1ccc2c(-c3ccc(N(c4ccc5c(ccc6ccccc65)c4)c4cccc5sc6ccccc6c45)c4c3oc3ccccc34)cccc2c1 ; c1ccc2c(-c3ccc(N(c4ccc5c(ccc6ccccc65)c4)c4ccc5sc6ccccc6c5c4)c4c3oc3ccccc34)cccc2c1] 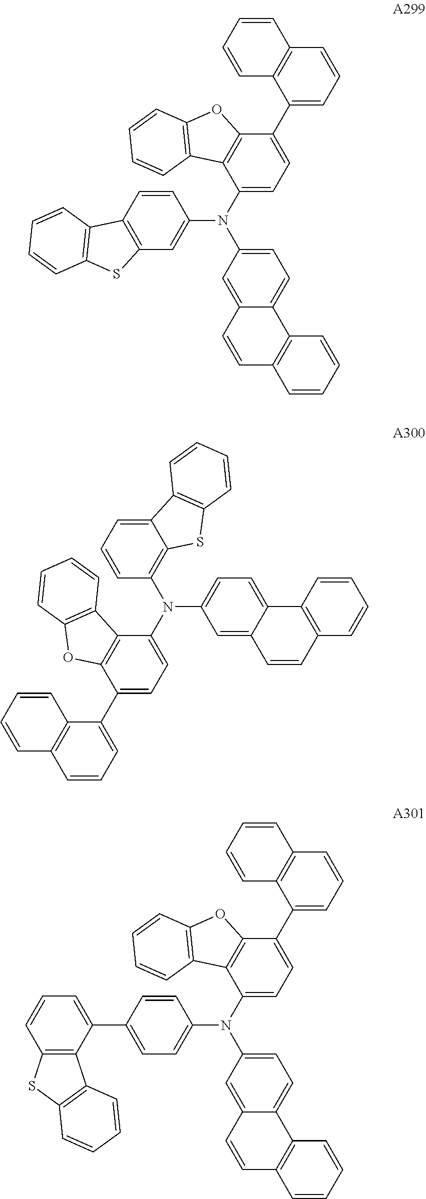 [SMILES: c1ccc2c(-c3ccc(N(c4ccc5c(ccc6ccccc65)c4)c4ccc5c(c4)sc4ccccc45)c4c3oc3ccccc34)cccc2c1 ; c1ccc2c(-c3ccc(N(c4ccc5c(ccc6ccccc65)c4)c4cccc5c4sc4ccccc45)c4c3oc3ccccc34)cccc2c1 ; c1ccc2c(-c3ccc(N(c4ccc(-c5cccc6sc7ccccc7c56)cc4)c4ccc5c(ccc6ccccc65)c4)c4c3oc3ccccc34)cccc2c1] 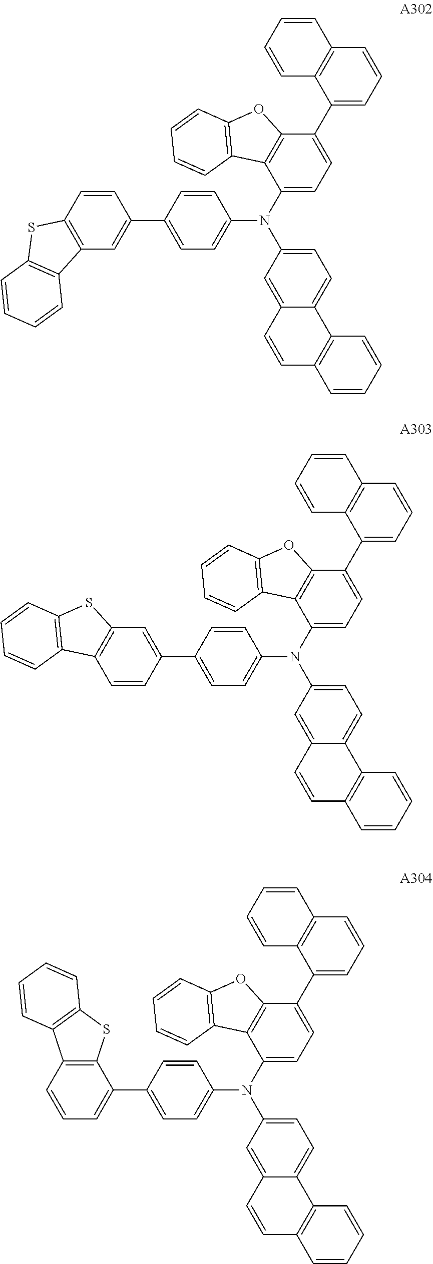 [SMILES: c1ccc2c(-c3ccc(N(c4ccc(-c5ccc6sc7ccccc7c6c5)cc4)c4ccc5c(ccc6ccccc65)c4)c4c3oc3ccccc34)cccc2c1 ; c1ccc2c(-c3ccc(N(c4ccc(-c5ccc6c(c5)sc5ccccc56)cc4)c4ccc5c(ccc6ccccc65)c4)c4c3oc3ccccc34)cccc2c1 ; c1ccc2c(-c3ccc(N(c4ccc(-c5cccc6c5sc5ccccc56)cc4)c4ccc5c(ccc6ccccc65)c4)c4c3oc3ccccc34)cccc2c1] 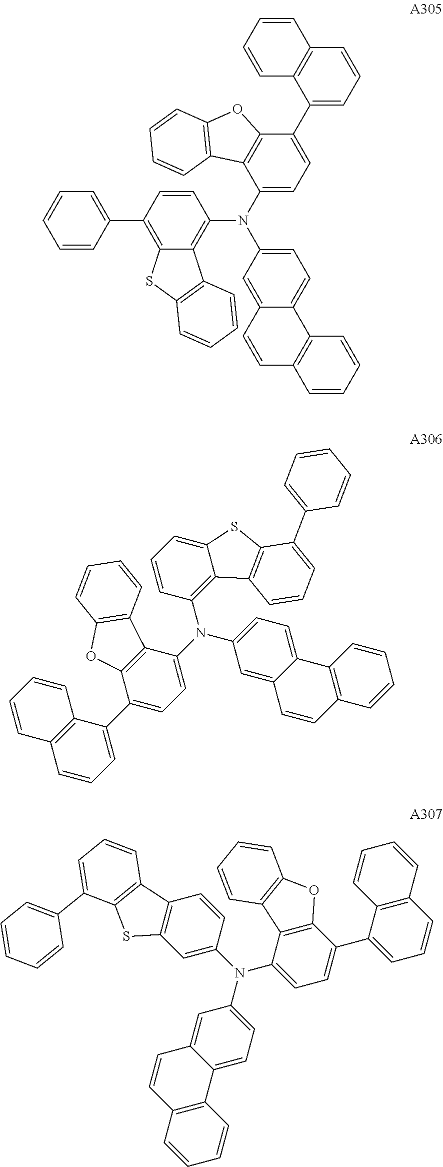 [SMILES: c1ccc(-c2ccc(N(c3ccc4c(ccc5ccccc54)c3)c3ccc(-c4cccc5ccccc45)c4oc5ccccc5c34)c3c2sc2ccccc23)cc1 ; c1ccc(-c2cccc3c2sc2cccc(N(c4ccc5c(ccc6ccccc65)c4)c4ccc(-c5cccc6ccccc56)c5oc6ccccc6c45)c23)cc1 ; c1ccc(-c2cccc3c2sc2cc(N(c4ccc5c(ccc6ccccc65)c4)c4ccc(-c5cccc6ccccc56)c5oc6ccccc6c45)ccc23)cc1] 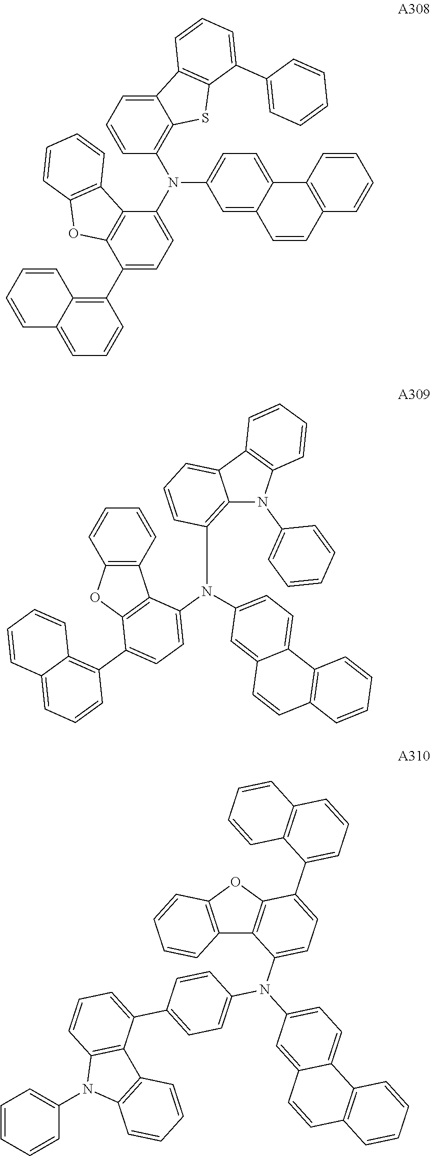 [SMILES: c1ccc(-c2cccc3c2sc2c(N(c4ccc5c(ccc6ccccc65)c4)c4ccc(-c5cccc6ccccc56)c5oc6ccccc6c45)cccc23)cc1 ; c1ccc(-n2c3ccccc3c3cccc(N(c4ccc5c(ccc6ccccc65)c4)c4ccc(-c5cccc6ccccc56)c5oc6ccccc6c45)c32)cc1 ; c1ccc(-n2c3ccccc3c3c(-c4ccc(N(c5ccc6c(ccc7ccccc76)c5)c5ccc(-c6cccc7ccccc67)c6oc7ccccc7c56)cc4)cccc32)cc1] 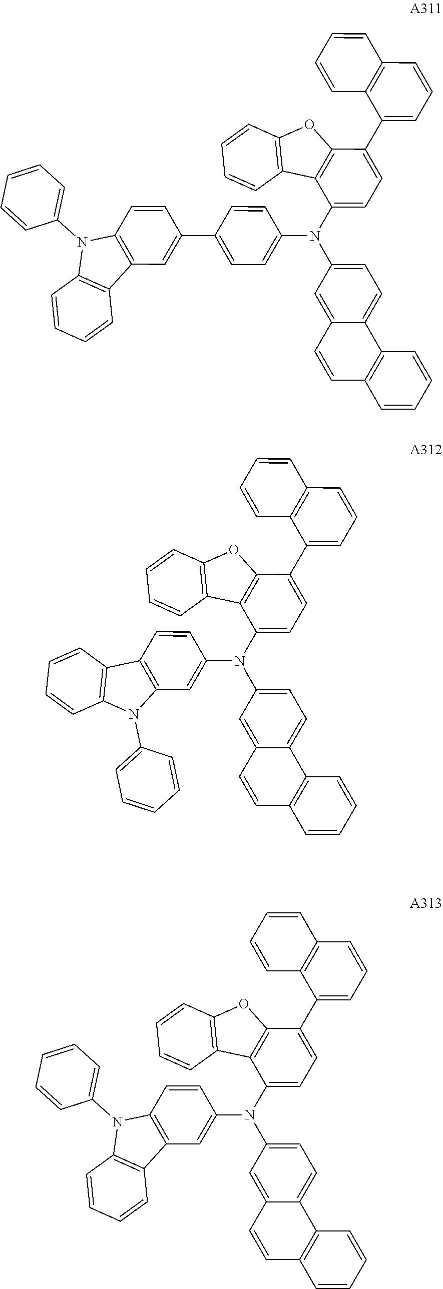 [SMILES: c1ccc(-n2c3ccccc3c3cc(-c4ccc(N(c5ccc6c(ccc7ccccc76)c5)c5ccc(-c6cccc7ccccc67)c6oc7ccccc7c56)cc4)ccc32)cc1 ; c1ccc(-n2c3ccccc3c3ccc(N(c4ccc5c(ccc6ccccc65)c4)c4ccc(-c5cccc6ccccc56)c5oc6ccccc6c45)cc32)cc1 ; c1ccc(-n2c3ccccc3c3cc(N(c4ccc5c(ccc6ccccc65)c4)c4ccc(-c5cccc6ccccc56)c5oc6ccccc6c45)ccc32)cc1] 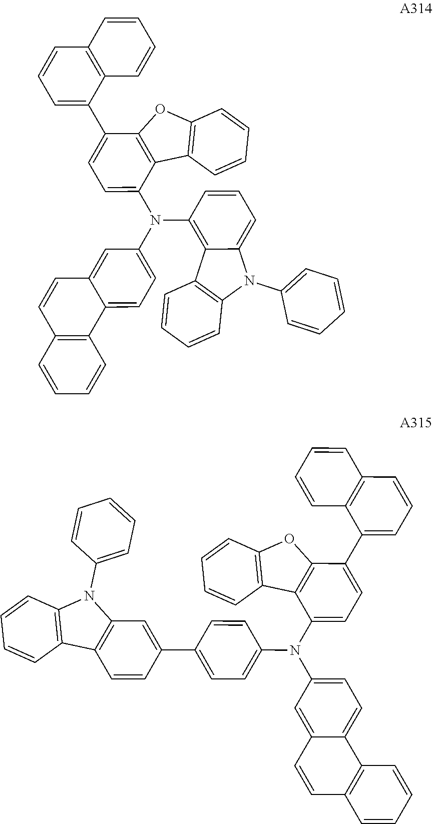 [SMILES: c1ccc(-n2c3ccccc3c3c(N(c4ccc5c(ccc6ccccc65)c4)c4ccc(-c5cccc6ccccc56)c5oc6ccccc6c45)cccc32)cc1 ; c1ccc(-n2c3ccccc3c3ccc(-c4ccc(N(c5ccc6c(ccc7ccccc76)c5)c5ccc(-c6cccc7ccccc67)c6oc7ccccc7c56)cc4)cc32)cc1] 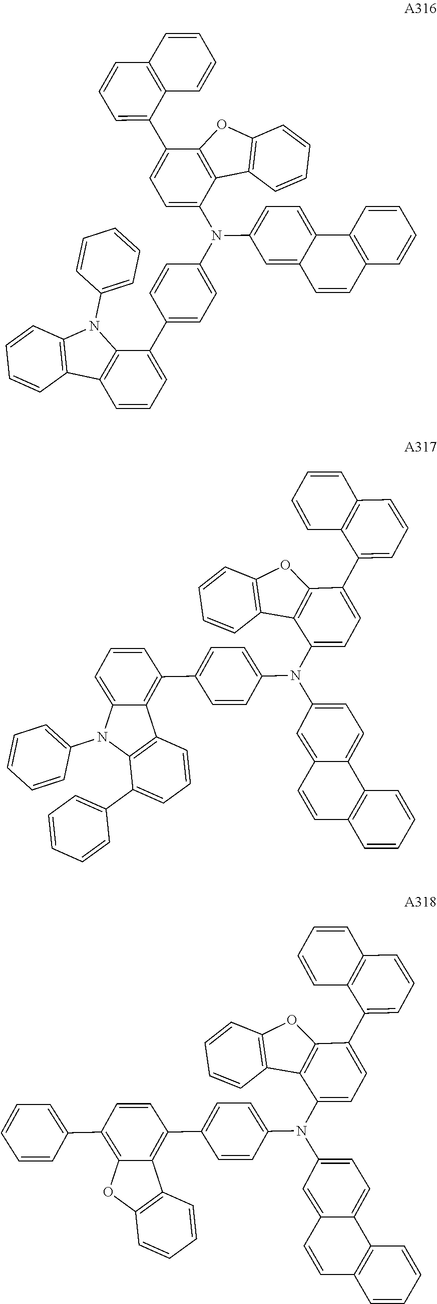 [SMILES: c1ccc(-n2c3ccccc3c3cccc(-c4ccc(N(c5ccc6c(ccc7ccccc76)c5)c5ccc(-c6cccc7ccccc67)c6oc7ccccc7c56)cc4)c32)cc1 ; c1ccc(-c2cccc3c4c(-c5ccc(N(c6ccc7c(ccc8ccccc87)c6)c6ccc(-c7cccc8ccccc78)c7oc8ccccc8c67)cc5)cccc4n(-c4ccccc4)c23)cc1 ; c1ccc(-c2ccc(-c3ccc(N(c4ccc5c(ccc6ccccc65)c4)c4ccc(-c5cccc6ccccc56)c5oc6ccccc6c45)cc3)c3c2oc2ccccc23)cc1] 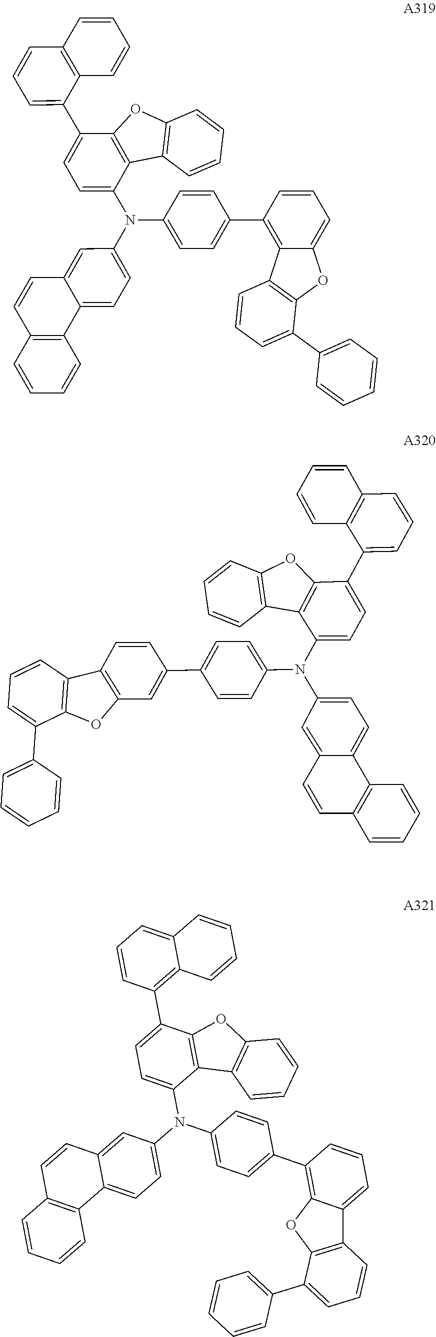 [SMILES: c1ccc(-c2cccc3c2oc2cccc(-c4ccc(N(c5ccc6c(ccc7ccccc76)c5)c5ccc(-c6cccc7ccccc67)c6oc7ccccc7c56)cc4)c23)cc1 ; c1ccc(-c2cccc3c2oc2cc(-c4ccc(N(c5ccc6c(ccc7ccccc76)c5)c5ccc(-c6cccc7ccccc67)c6oc7ccccc7c56)cc4)ccc23)cc1 ; c1ccc(-c2cccc3c2oc2c(-c4ccc(N(c5ccc6c(ccc7ccccc76)c5)c5ccc(-c6cccc7ccccc67)c6oc7ccccc7c56)cc4)cccc23)cc1] 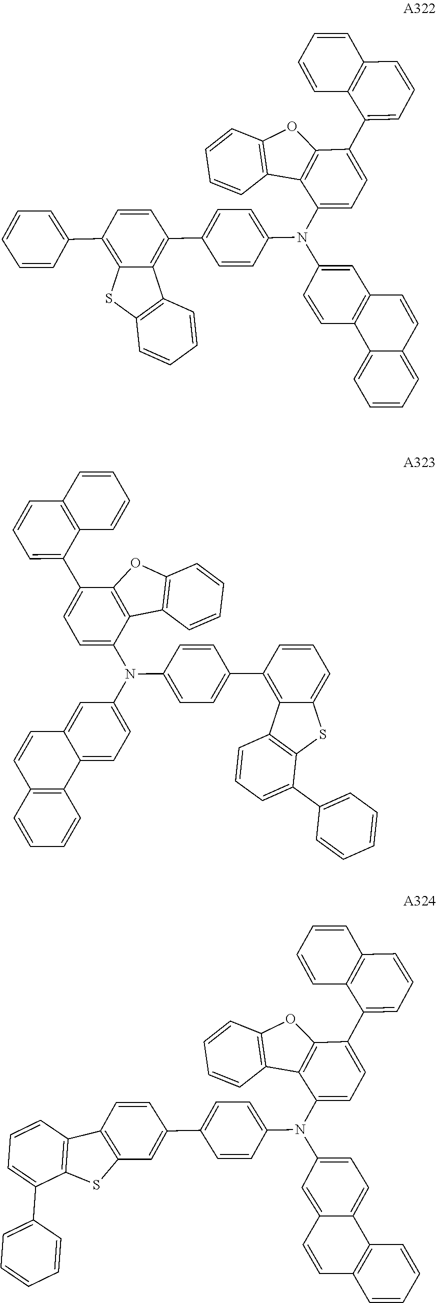 [SMILES: c1ccc(-c2ccc(-c3ccc(N(c4ccc5c(ccc6ccccc65)c4)c4ccc(-c5cccc6ccccc56)c5oc6ccccc6c45)cc3)c3c2sc2ccccc23)cc1 ; c1ccc(-c2cccc3c2sc2cccc(-c4ccc(N(c5ccc6c(ccc7ccccc76)c5)c5ccc(-c6cccc7ccccc67)c6oc7ccccc7c56)cc4)c23)cc1 ; c1ccc(-c2cccc3c2sc2cc(-c4ccc(N(c5ccc6c(ccc7ccccc76)c5)c5ccc(-c6cccc7ccccc67)c6oc7ccccc7c56)cc4)ccc23)cc1] 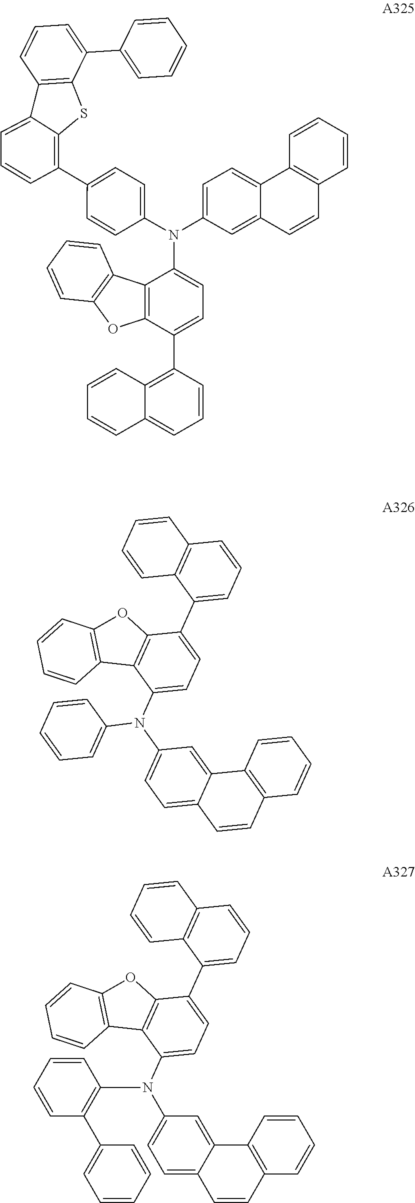 [SMILES: c1ccc(-c2cccc3c2sc2c(-c4ccc(N(c5ccc6c(ccc7ccccc76)c5)c5ccc(-c6cccc7ccccc67)c6oc7ccccc7c56)cc4)cccc23)cc1 ; c1ccc(N(c2ccc3ccc4ccccc4c3c2)c2ccc(-c3cccc4ccccc34)c3oc4ccccc4c23)cc1 ; c1ccc(-c2ccccc2N(c2ccc3ccc4ccccc4c3c2)c2ccc(-c3cccc4ccccc34)c3oc4ccccc4c23)cc1] 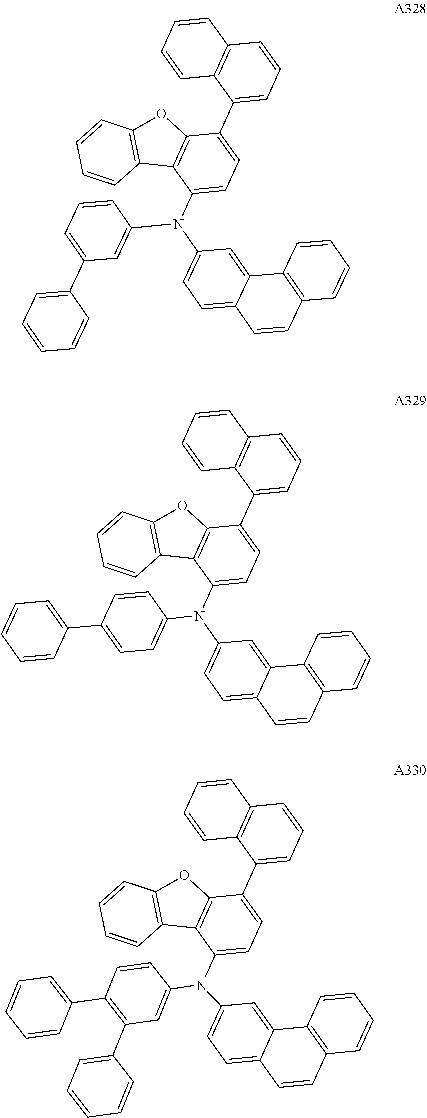 [SMILES: c1ccc(-c2cccc(N(c3ccc4ccc5ccccc5c4c3)c3ccc(-c4cccc5ccccc45)c4oc5ccccc5c34)c2)cc1 ; c1ccc(-c2ccc(N(c3ccc4ccc5ccccc5c4c3)c3ccc(-c4cccc5ccccc45)c4oc5ccccc5c34)cc2)cc1 ; c1ccc(-c2ccc(N(c3ccc4ccc5ccccc5c4c3)c3ccc(-c4cccc5ccccc45)c4oc5ccccc5c34)cc2-c2ccccc2)cc1] 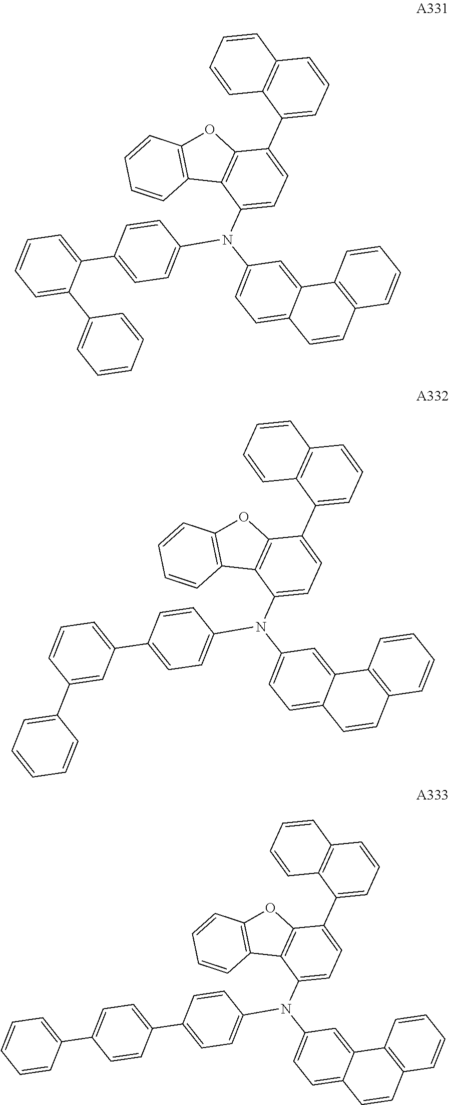 [SMILES: c1ccc(-c2ccccc2-c2ccc(N(c3ccc4ccc5ccccc5c4c3)c3ccc(-c4cccc5ccccc45)c4oc5ccccc5c34)cc2)cc1 ; c1ccc(-c2cccc(-c3ccc(N(c4ccc5ccc6ccccc6c5c4)c4ccc(-c5cccc6ccccc56)c5oc6ccccc6c45)cc3)c2)cc1 ; c1ccc(-c2ccc(-c3ccc(N(c4ccc5ccc6ccccc6c5c4)c4ccc(-c5cccc6ccccc56)c5oc6ccccc6c45)cc3)cc2)cc1] 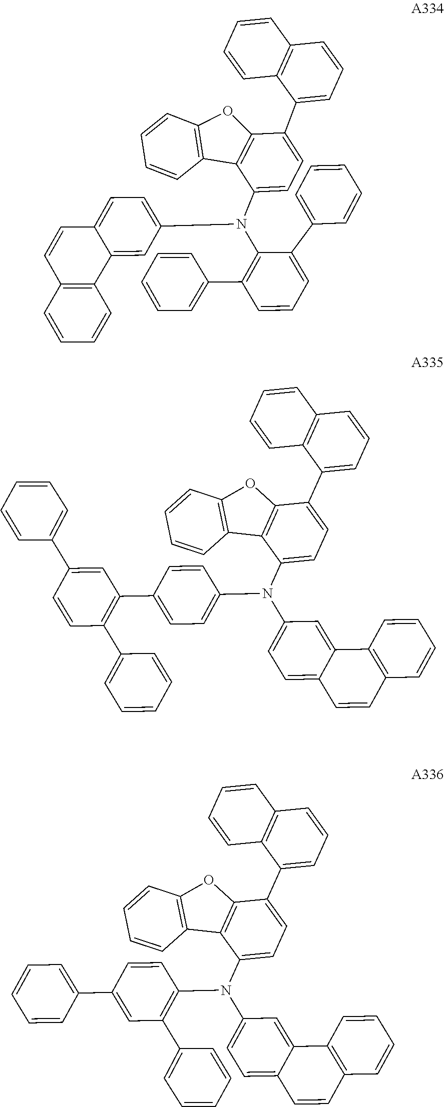 [SMILES: c1ccc(-c2cccc(-c3ccccc3)c2N(c2ccc3ccc4ccccc4c3c2)c2ccc(-c3cccc4ccccc34)c3oc4ccccc4c23)cc1 ; c1ccc(-c2ccc(-c3ccccc3)c(-c3ccc(N(c4ccc5ccc6ccccc6c5c4)c4ccc(-c5cccc6ccccc56)c5oc6ccccc6c45)cc3)c2)cc1 ; c1ccc(-c2ccc(N(c3ccc4ccc5ccccc5c4c3)c3ccc(-c4cccc5ccccc45)c4oc5ccccc5c34)c(-c3ccccc3)c2)cc1] 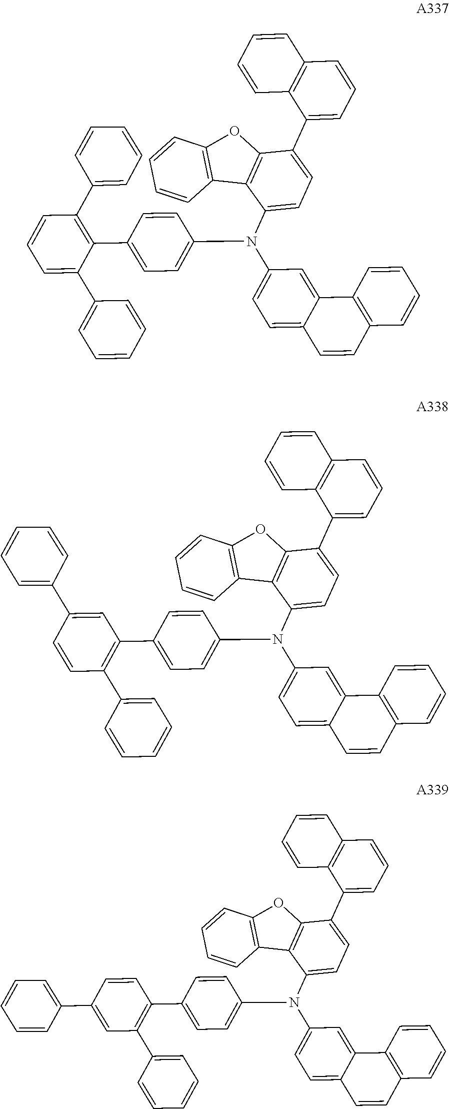 [SMILES: c1ccc(-c2cccc(-c3ccccc3)c2-c2ccc(N(c3ccc4ccc5ccccc5c4c3)c3ccc(-c4cccc5ccccc45)c4oc5ccccc5c34)cc2)cc1 ; c1ccc(-c2ccc(-c3ccccc3)c(-c3ccc(N(c4ccc5ccc6ccccc6c5c4)c4ccc(-c5cccc6ccccc56)c5oc6ccccc6c45)cc3)c2)cc1 ; c1ccc(-c2ccc(-c3ccc(N(c4ccc5ccc6ccccc6c5c4)c4ccc(-c5cccc6ccccc56)c5oc6ccccc6c45)cc3)c(-c3ccccc3)c2)cc1] 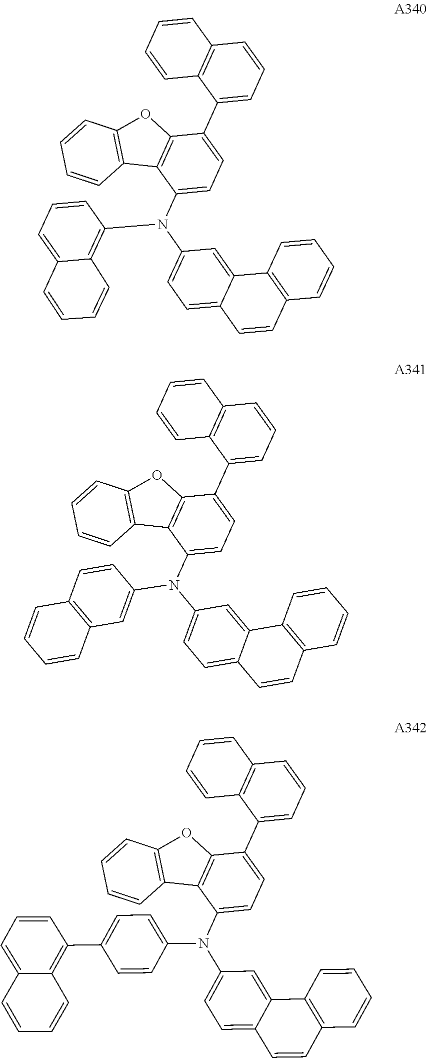 [SMILES: c1ccc2c(-c3ccc(N(c4ccc5ccc6ccccc6c5c4)c4cccc5ccccc45)c4c3oc3ccccc34)cccc2c1 ; c1ccc2cc(N(c3ccc4ccc5ccccc5c4c3)c3ccc(-c4cccc5ccccc45)c4oc5ccccc5c34)ccc2c1 ; c1ccc2c(-c3ccc(N(c4ccc5ccc6ccccc6c5c4)c4ccc(-c5cccc6ccccc56)c5oc6ccccc6c45)cc3)cccc2c1] 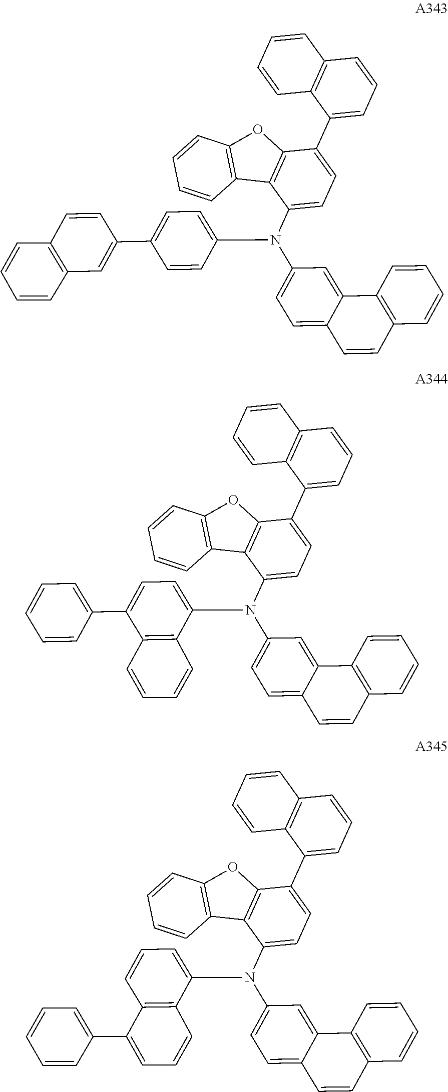 [SMILES: c1ccc2cc(-c3ccc(N(c4ccc5ccc6ccccc6c5c4)c4ccc(-c5cccc6ccccc56)c5oc6ccccc6c45)cc3)ccc2c1 ; c1ccc(-c2ccc(N(c3ccc4ccc5ccccc5c4c3)c3ccc(-c4cccc5ccccc45)c4oc5ccccc5c34)c3ccccc23)cc1 ; c1ccc(-c2cccc3c(N(c4ccc5ccc6ccccc6c5c4)c4ccc(-c5cccc6ccccc56)c5oc6ccccc6c45)cccc23)cc1] 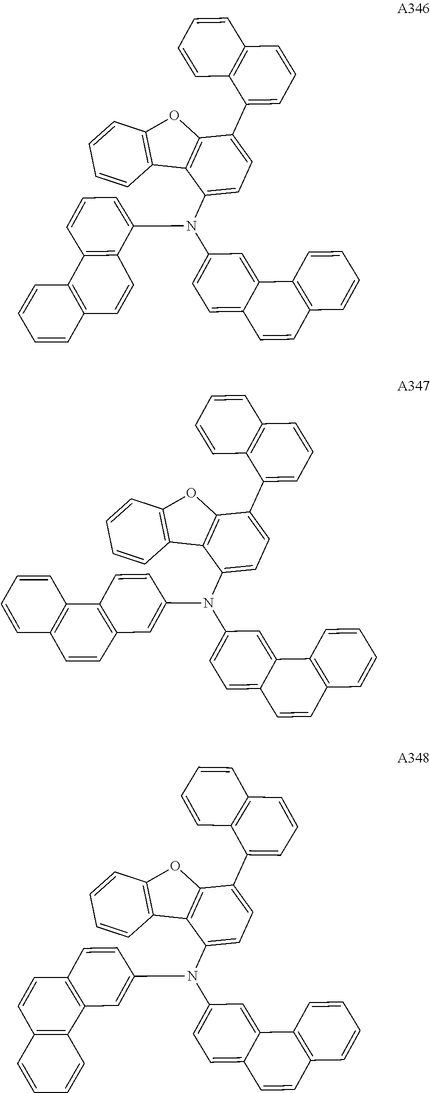 [SMILES: c1ccc2c(-c3ccc(N(c4ccc5ccc6ccccc6c5c4)c4cccc5c4ccc4ccccc45)c4c3oc3ccccc34)cccc2c1 ; c1ccc2c(-c3ccc(N(c4ccc5c(ccc6ccccc65)c4)c4ccc5ccc6ccccc6c5c4)c4c3oc3ccccc34)cccc2c1 ; c1ccc2c(-c3ccc(N(c4ccc5ccc6ccccc6c5c4)c4ccc5ccc6ccccc6c5c4)c4c3oc3ccccc34)cccc2c1] 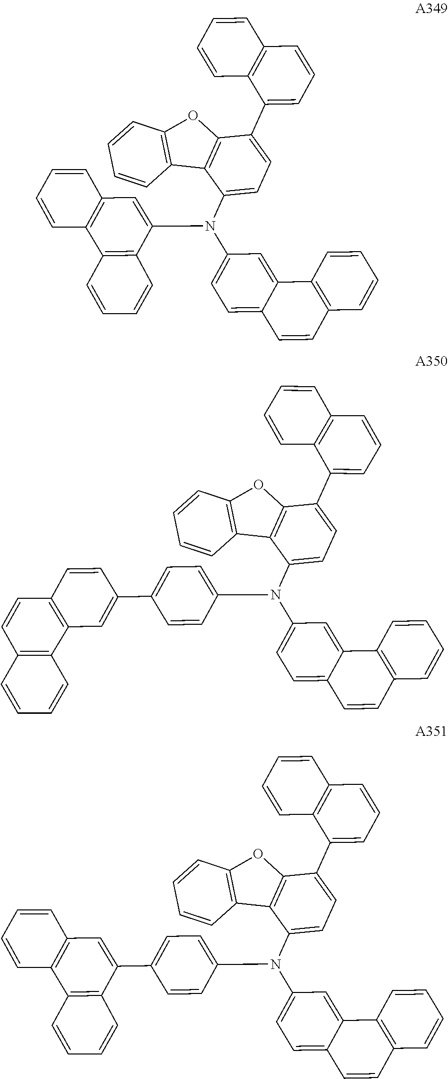 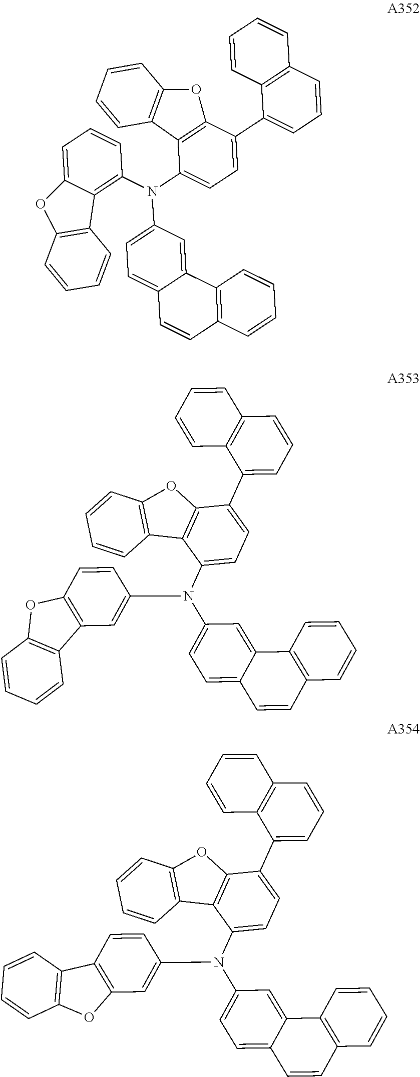 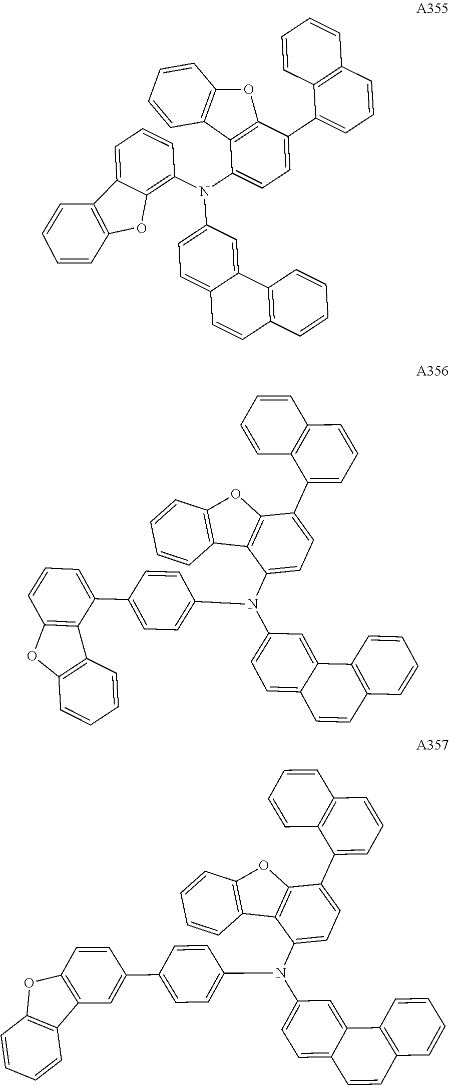 [SMILES: c1ccc2c(-c3ccc(N(c4ccc5ccc6ccccc6c5c4)c4cccc5c4oc4ccccc45)c4c3oc3ccccc34)cccc2c1 ; c1ccc2c(-c3ccc(N(c4ccc(-c5cccc6oc7ccccc7c56)cc4)c4ccc5ccc6ccccc6c5c4)c4c3oc3ccccc34)cccc2c1 ; c1ccc2c(-c3ccc(N(c4ccc(-c5ccc6oc7ccccc7c6c5)cc4)c4ccc5ccc6ccccc6c5c4)c4c3oc3ccccc34)cccc2c1] 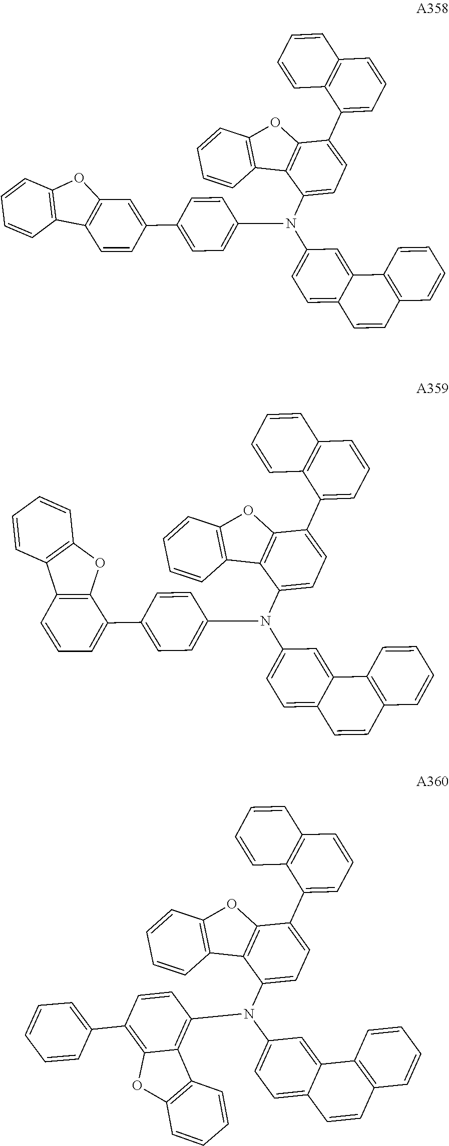 [SMILES: c1ccc2c(-c3ccc(N(c4ccc(-c5ccc6c(c5)oc5ccccc56)cc4)c4ccc5ccc6ccccc6c5c4)c4c3oc3ccccc34)cccc2c1 ; c1ccc2c(-c3ccc(N(c4ccc(-c5cccc6c5oc5ccccc56)cc4)c4ccc5ccc6ccccc6c5c4)c4c3oc3ccccc34)cccc2c1 ; c1ccc(-c2ccc(N(c3ccc4ccc5ccccc5c4c3)c3ccc(-c4cccc5ccccc45)c4oc5ccccc5c34)c3c2oc2ccccc23)cc1] 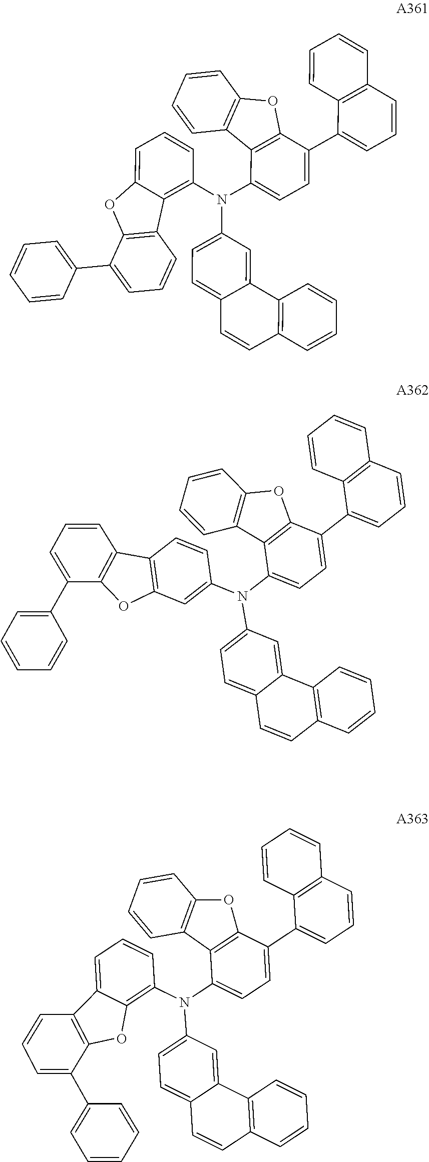 [SMILES: c1ccc(-c2cccc3c2oc2cccc(N(c4ccc5ccc6ccccc6c5c4)c4ccc(-c5cccc6ccccc56)c5oc6ccccc6c45)c23)cc1 ; c1ccc(-c2cccc3c2oc2cc(N(c4ccc5ccc6ccccc6c5c4)c4ccc(-c5cccc6ccccc56)c5oc6ccccc6c45)ccc23)cc1 ; c1ccc(-c2cccc3c2oc2c(N(c4ccc5ccc6ccccc6c5c4)c4ccc(-c5cccc6ccccc56)c5oc6ccccc6c45)cccc23)cc1] 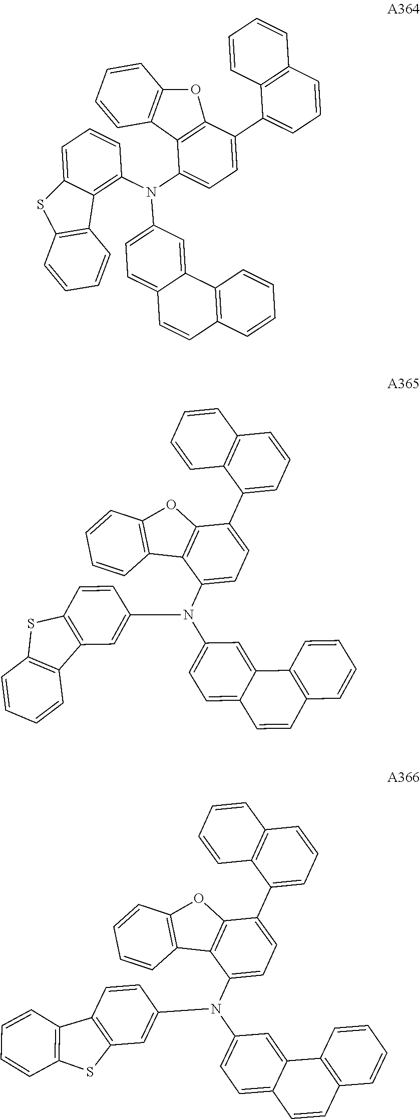 [SMILES: c1ccc2c(-c3ccc(N(c4ccc5ccc6ccccc6c5c4)c4cccc5sc6ccccc6c45)c4c3oc3ccccc34)cccc2c1 ; c1ccc2c(-c3ccc(N(c4ccc5ccc6ccccc6c5c4)c4ccc5sc6ccccc6c5c4)c4c3oc3ccccc34)cccc2c1 ; c1ccc2c(-c3ccc(N(c4ccc5c(c4)sc4ccccc45)c4ccc5ccc6ccccc6c5c4)c4c3oc3ccccc34)cccc2c1] 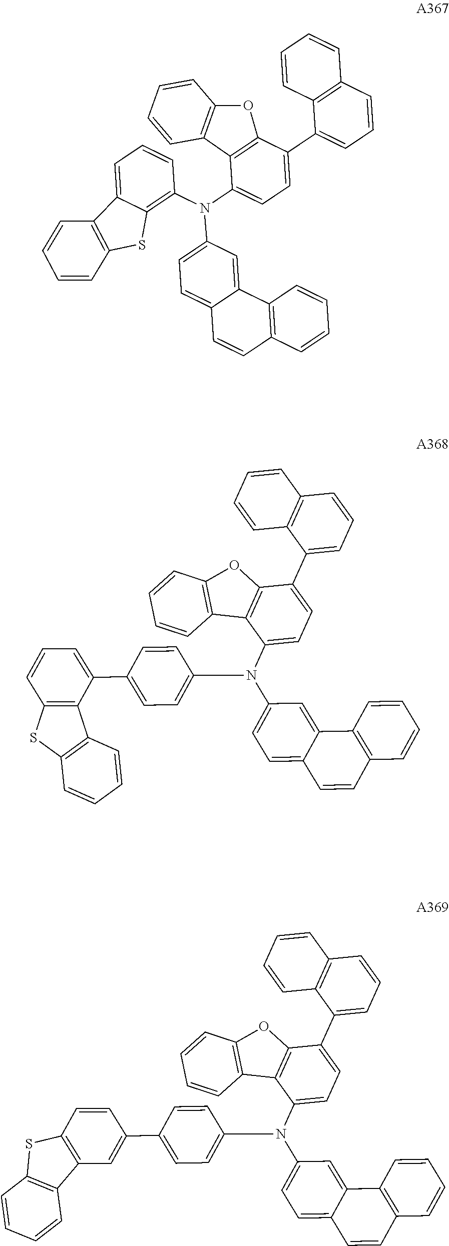 [SMILES: c1ccc2c(-c3ccc(N(c4ccc5ccc6ccccc6c5c4)c4cccc5c4sc4ccccc45)c4c3oc3ccccc34)cccc2c1 ; c1ccc2c(-c3ccc(N(c4ccc(-c5cccc6sc7ccccc7c56)cc4)c4ccc5ccc6ccccc6c5c4)c4c3oc3ccccc34)cccc2c1 ; c1ccc2c(-c3ccc(N(c4ccc(-c5ccc6sc7ccccc7c6c5)cc4)c4ccc5ccc6ccccc6c5c4)c4c3oc3ccccc34)cccc2c1] 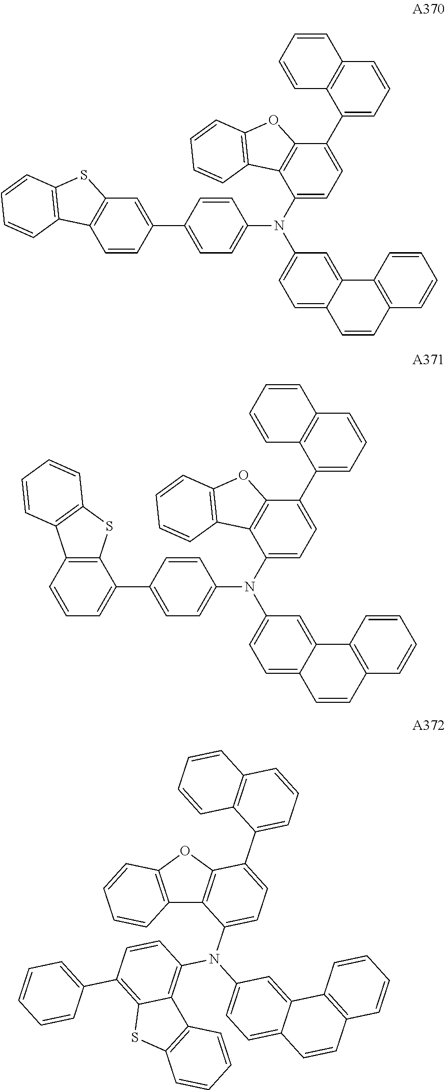 [SMILES: c1ccc2c(-c3ccc(N(c4ccc(-c5ccc6c(c5)sc5ccccc56)cc4)c4ccc5ccc6ccccc6c5c4)c4c3oc3ccccc34)cccc2c1 ; c1ccc2c(-c3ccc(N(c4ccc(-c5cccc6c5sc5ccccc56)cc4)c4ccc5ccc6ccccc6c5c4)c4c3oc3ccccc34)cccc2c1 ; c1ccc(-c2ccc(N(c3ccc4ccc5ccccc5c4c3)c3ccc(-c4cccc5ccccc45)c4oc5ccccc5c34)c3c2sc2ccccc23)cc1] 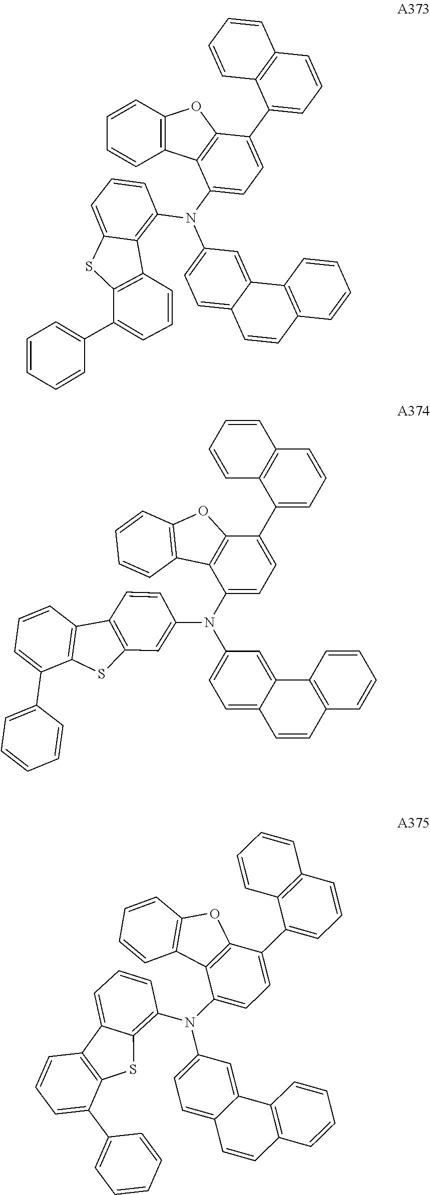 [SMILES: c1ccc(-c2cccc3c2sc2cccc(N(c4ccc5ccc6ccccc6c5c4)c4ccc(-c5cccc6ccccc56)c5oc6ccccc6c45)c23)cc1 ; c1ccc(-c2cccc3c2sc2cc(N(c4ccc5ccc6ccccc6c5c4)c4ccc(-c5cccc6ccccc56)c5oc6ccccc6c45)ccc23)cc1 ; c1ccc(-c2cccc3c2sc2c(N(c4ccc5ccc6ccccc6c5c4)c4ccc(-c5cccc6ccccc56)c5oc6ccccc6c45)cccc23)cc1] 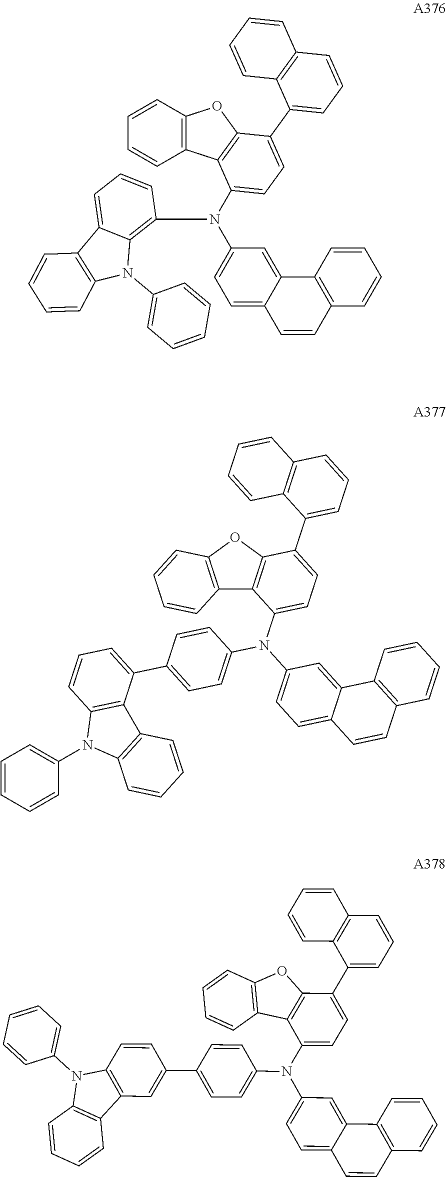 [SMILES: c1ccc(-n2c3ccccc3c3cccc(N(c4ccc5ccc6ccccc6c5c4)c4ccc(-c5cccc6ccccc56)c5oc6ccccc6c45)c32)cc1 ; c1ccc(-n2c3ccccc3c3c(-c4ccc(N(c5ccc6ccc7ccccc7c6c5)c5ccc(-c6cccc7ccccc67)c6oc7ccccc7c56)cc4)cccc32)cc1 ; c1ccc(-n2c3ccccc3c3cc(-c4ccc(N(c5ccc6ccc7ccccc7c6c5)c5ccc(-c6cccc7ccccc67)c6oc7ccccc7c56)cc4)ccc32)cc1] 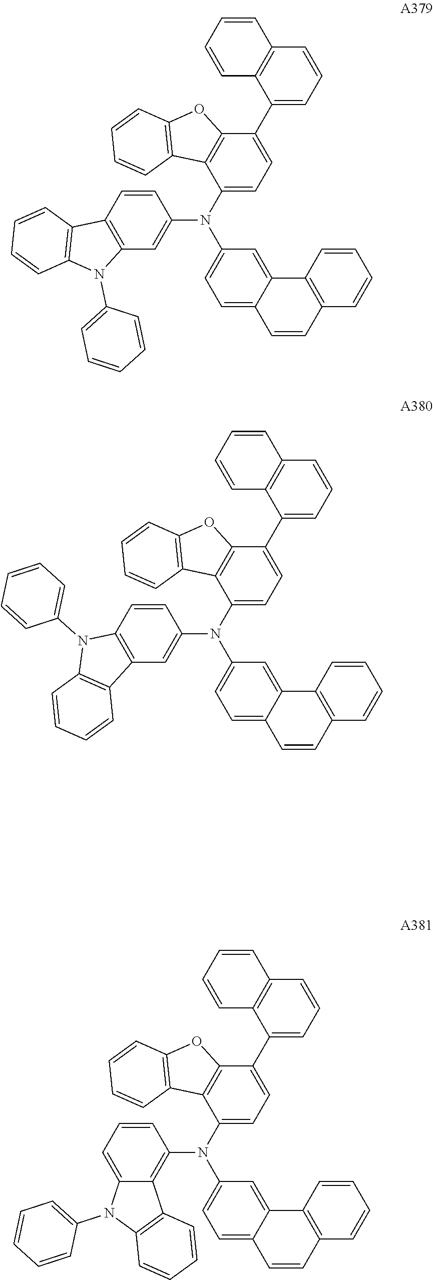 [SMILES: c1ccc(-n2c3ccccc3c3ccc(N(c4ccc5ccc6ccccc6c5c4)c4ccc(-c5cccc6ccccc56)c5oc6ccccc6c45)cc32)cc1 ; c1ccc(-n2c3ccccc3c3cc(N(c4ccc5ccc6ccccc6c5c4)c4ccc(-c5cccc6ccccc56)c5oc6ccccc6c45)ccc32)cc1 ; c1ccc(-n2c3ccccc3c3c(N(c4ccc5ccc6ccccc6c5c4)c4ccc(-c5cccc6ccccc56)c5oc6ccccc6c45)cccc32)cc1] 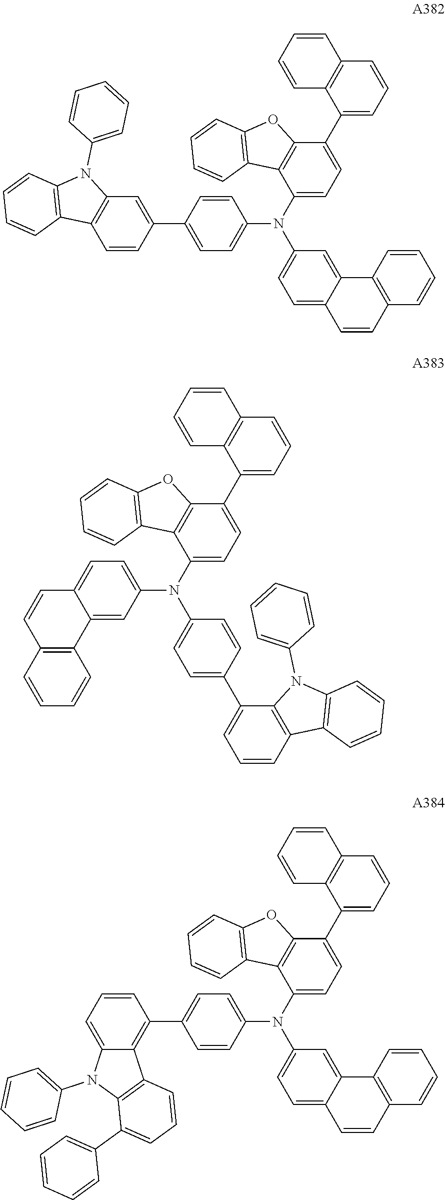 [SMILES: c1ccc(-n2c3ccccc3c3ccc(-c4ccc(N(c5ccc6ccc7ccccc7c6c5)c5ccc(-c6cccc7ccccc67)c6oc7ccccc7c56)cc4)cc32)cc1 ; c1ccc(-n2c3ccccc3c3cccc(-c4ccc(N(c5ccc6ccc7ccccc7c6c5)c5ccc(-c6cccc7ccccc67)c6oc7ccccc7c56)cc4)c32)cc1 ; c1ccc(-c2cccc3c4c(-c5ccc(N(c6ccc7ccc8ccccc8c7c6)c6ccc(-c7cccc8ccccc78)c7oc8ccccc8c67)cc5)cccc4n(-c4ccccc4)c23)cc1] 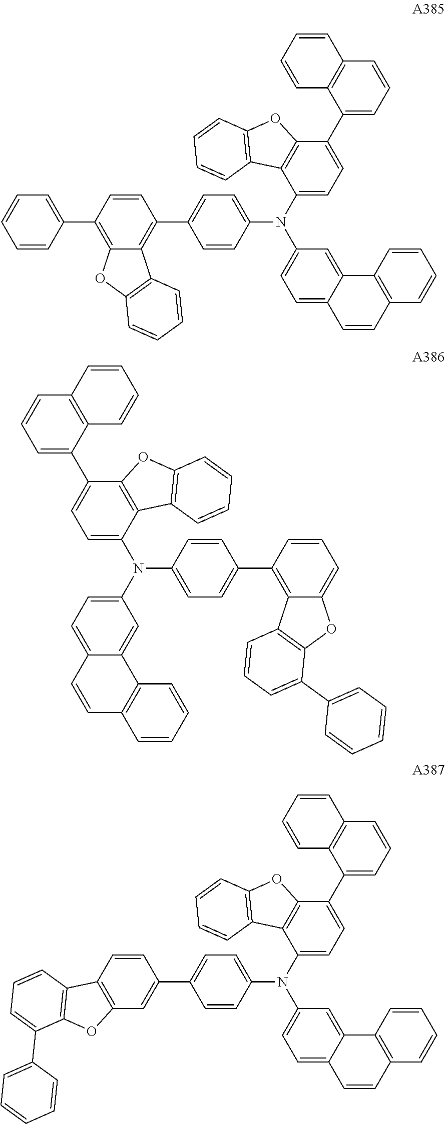 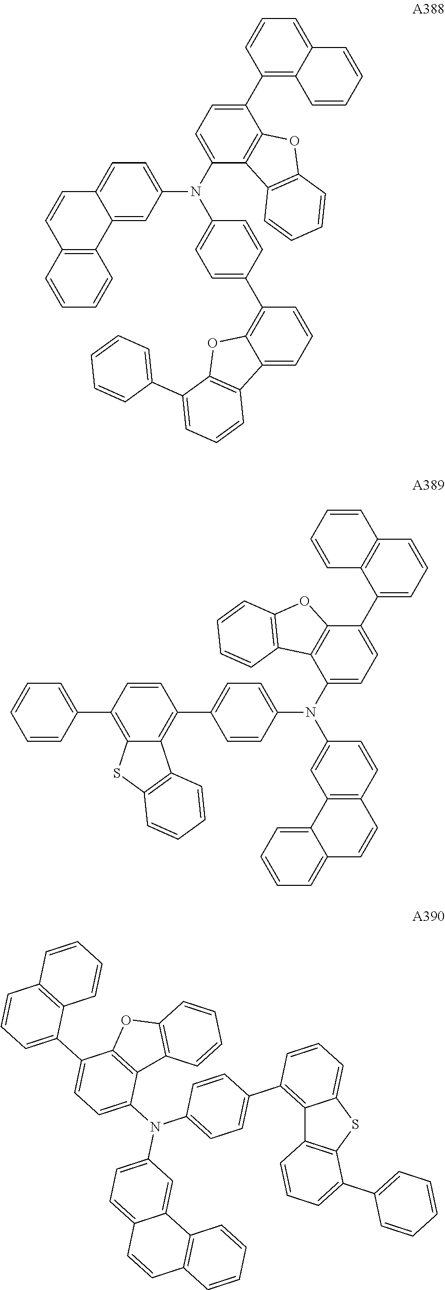 [SMILES: c1ccc(-c2cccc3c2oc2c(-c4ccc(N(c5ccc6ccc7ccccc7c6c5)c5ccc(-c6cccc7ccccc67)c6oc7ccccc7c56)cc4)cccc23)cc1 ; c1ccc(-c2ccc(-c3ccc(N(c4ccc5ccc6ccccc6c5c4)c4ccc(-c5cccc6ccccc56)c5oc6ccccc6c45)cc3)c3c2sc2ccccc23)cc1 ; c1ccc(-c2cccc3c2sc2cccc(-c4ccc(N(c5ccc6ccc7ccccc7c6c5)c5ccc(-c6cccc7ccccc67)c6oc7ccccc7c56)cc4)c23)cc1] 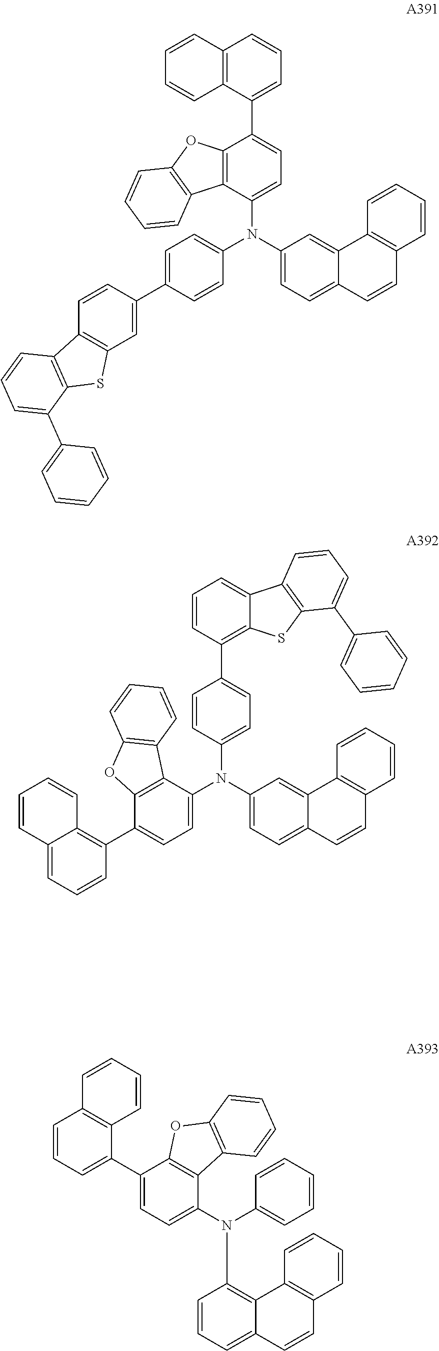 [SMILES: c1ccc(-c2cccc3c2sc2cc(-c4ccc(N(c5ccc6ccc7ccccc7c6c5)c5ccc(-c6cccc7ccccc67)c6oc7ccccc7c56)cc4)ccc23)cc1 ; c1ccc(-c2cccc3c2sc2c(-c4ccc(N(c5ccc6ccc7ccccc7c6c5)c5ccc(-c6cccc7ccccc67)c6oc7ccccc7c56)cc4)cccc23)cc1 ; c1ccc(N(c2cccc3ccc4ccccc4c23)c2ccc(-c3cccc4ccccc34)c3oc4ccccc4c23)cc1] 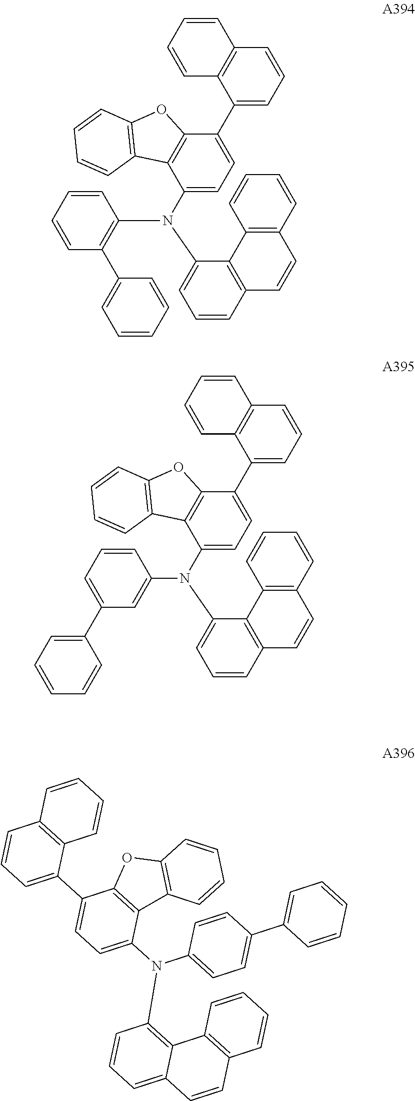 [SMILES: c1ccc(-c2ccccc2N(c2cccc3ccc4ccccc4c23)c2ccc(-c3cccc4ccccc34)c3oc4ccccc4c23)cc1 ; c1ccc(-c2cccc(N(c3cccc4ccc5ccccc5c34)c3ccc(-c4cccc5ccccc45)c4oc5ccccc5c34)c2)cc1 ; c1ccc(-c2ccc(N(c3cccc4ccc5ccccc5c34)c3ccc(-c4cccc5ccccc45)c4oc5ccccc5c34)cc2)cc1] 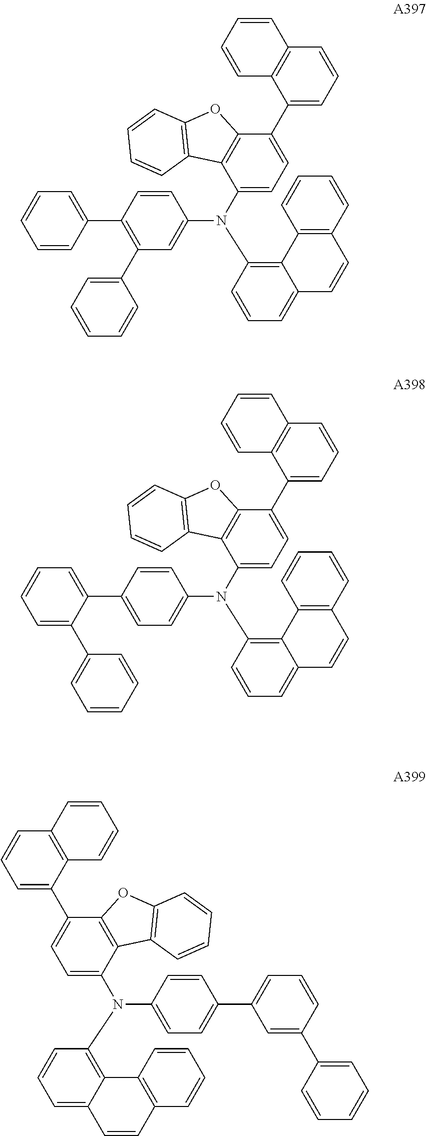 [SMILES: c1ccc(-c2ccc(N(c3cccc4ccc5ccccc5c34)c3ccc(-c4cccc5ccccc45)c4oc5ccccc5c34)cc2-c2ccccc2)cc1 ; c1ccc(-c2ccccc2-c2ccc(N(c3cccc4ccc5ccccc5c34)c3ccc(-c4cccc5ccccc45)c4oc5ccccc5c34)cc2)cc1 ; c1ccc(-c2cccc(-c3ccc(N(c4cccc5ccc6ccccc6c45)c4ccc(-c5cccc6ccccc56)c5oc6ccccc6c45)cc3)c2)cc1] 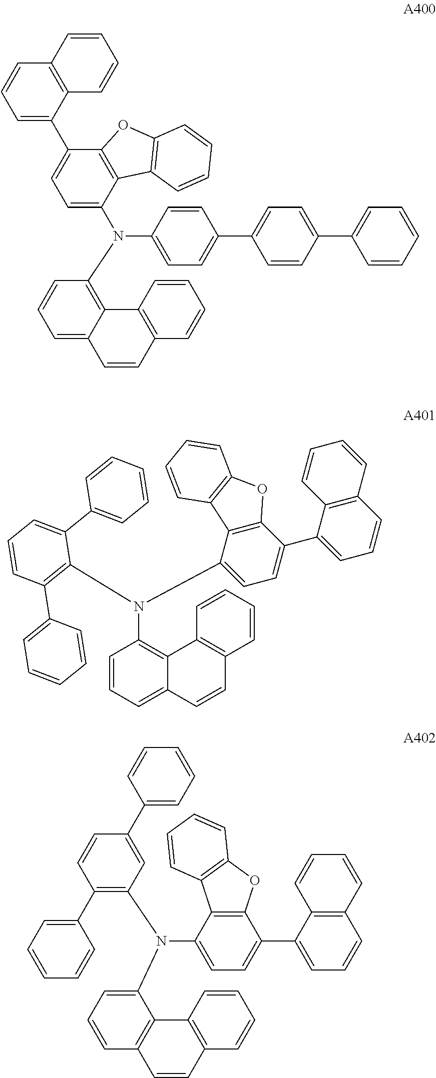 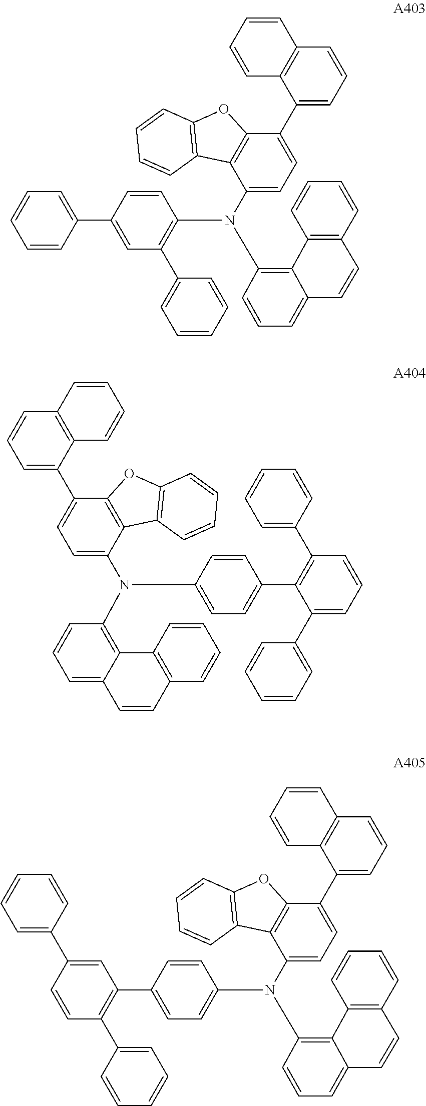 [SMILES: c1ccc(-c2ccc(N(c3cccc4ccc5ccccc5c34)c3ccc(-c4cccc5ccccc45)c4oc5ccccc5c34)c(-c3ccccc3)c2)cc1 ; c1ccc(-c2cccc(-c3ccccc3)c2-c2ccc(N(c3cccc4ccc5ccccc5c34)c3ccc(-c4cccc5ccccc45)c4oc5ccccc5c34)cc2)cc1 ; c1ccc(-c2ccc(-c3ccccc3)c(-c3ccc(N(c4cccc5ccc6ccccc6c45)c4ccc(-c5cccc6ccccc56)c5oc6ccccc6c45)cc3)c2)cc1] 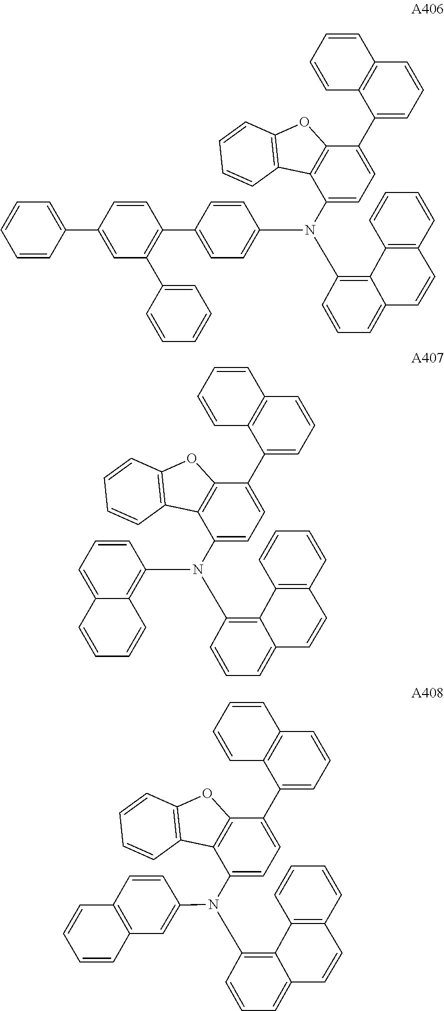 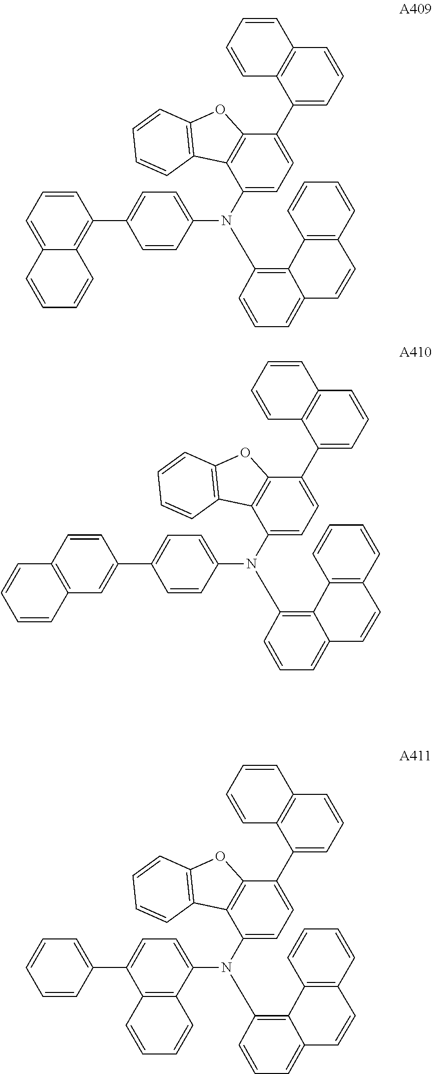 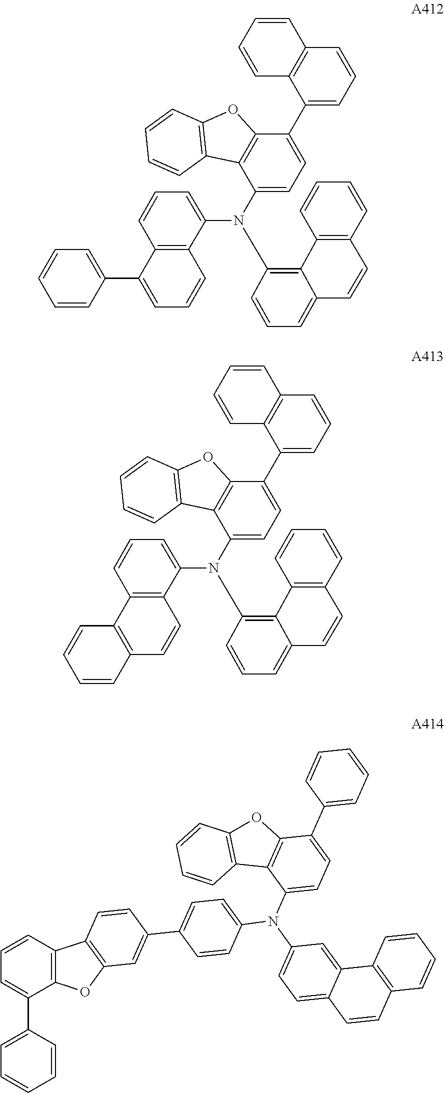 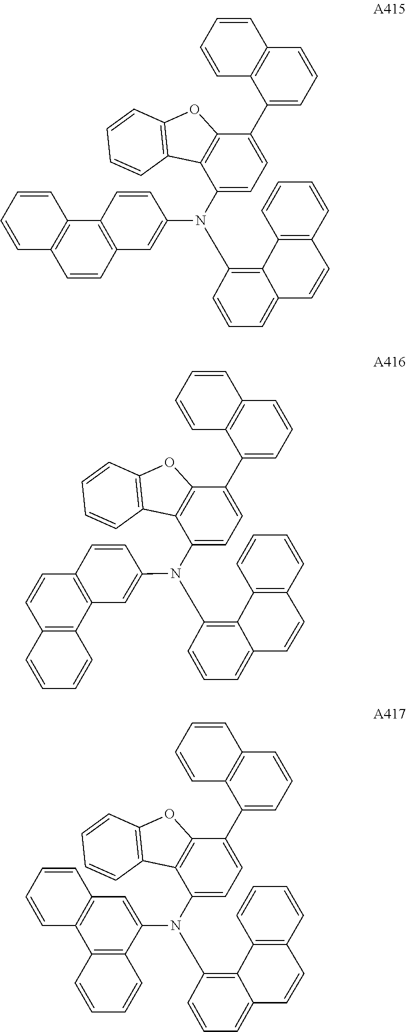 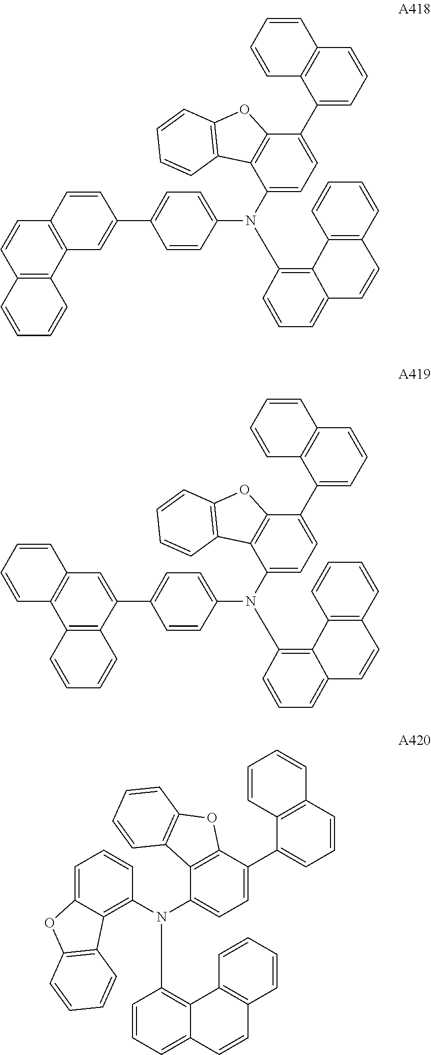 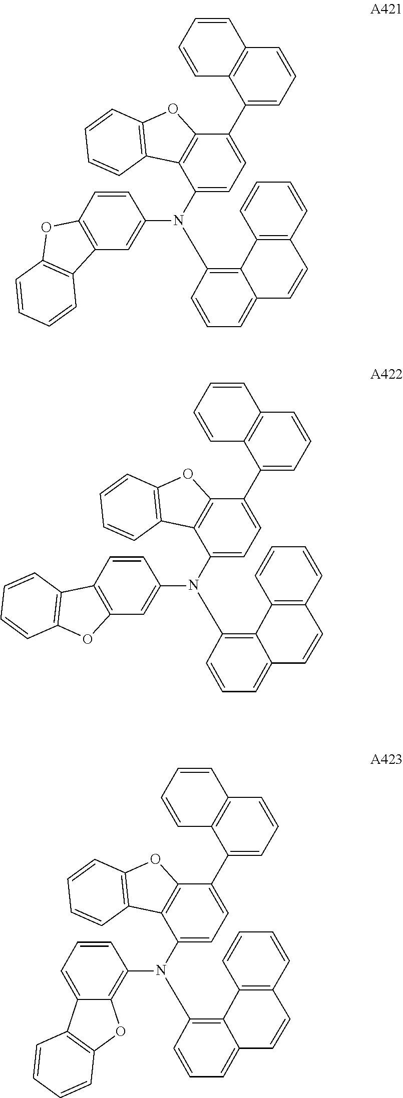 [SMILES: c1ccc2c(-c3ccc(N(c4ccc5oc6ccccc6c5c4)c4cccc5ccc6ccccc6c45)c4c3oc3ccccc34)cccc2c1 ; c1ccc2c(-c3ccc(N(c4ccc5c(c4)oc4ccccc45)c4cccc5ccc6ccccc6c45)c4c3oc3ccccc34)cccc2c1 ; c1ccc2c(-c3ccc(N(c4cccc5c4oc4ccccc45)c4cccc5ccc6ccccc6c45)c4c3oc3ccccc34)cccc2c1] 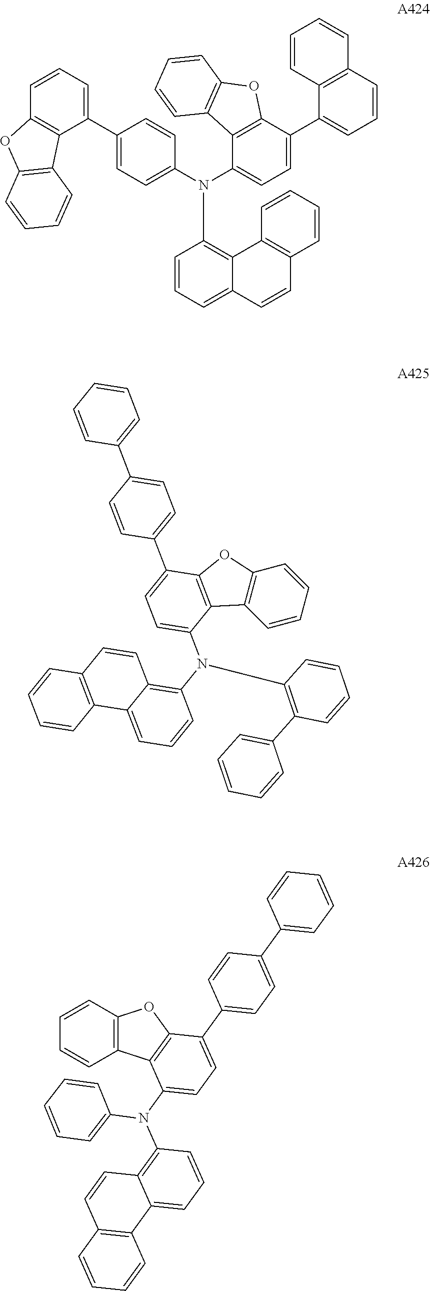 [SMILES: c1ccc2c(-c3ccc(N(c4ccc(-c5cccc6oc7ccccc7c56)cc4)c4cccc5ccc6ccccc6c45)c4c3oc3ccccc34)cccc2c1 ; c1ccc(-c2ccc(-c3ccc(N(c4ccccc4-c4ccccc4)c4cccc5c4ccc4ccccc45)c4c3oc3ccccc34)cc2)cc1 ; c1ccc(-c2ccc(-c3ccc(N(c4ccccc4)c4cccc5c4ccc4ccccc45)c4c3oc3ccccc34)cc2)cc1] 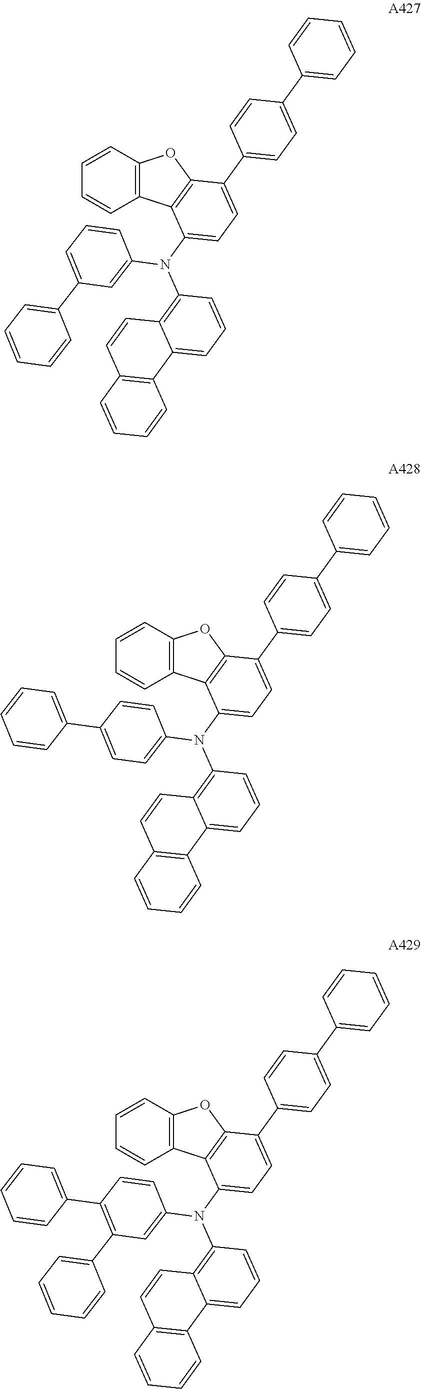 [SMILES: c1ccc(-c2ccc(-c3ccc(N(c4cccc(-c5ccccc5)c4)c4cccc5c4ccc4ccccc45)c4c3oc3ccccc34)cc2)cc1 ; c1ccc(-c2ccc(-c3ccc(N(c4ccc(-c5ccccc5)cc4)c4cccc5c4ccc4ccccc45)c4c3oc3ccccc34)cc2)cc1 ; c1ccc(-c2ccc(-c3ccc(N(c4ccc(-c5ccccc5)c(-c5ccccc5)c4)c4cccc5c4ccc4ccccc45)c4c3oc3ccccc34)cc2)cc1] 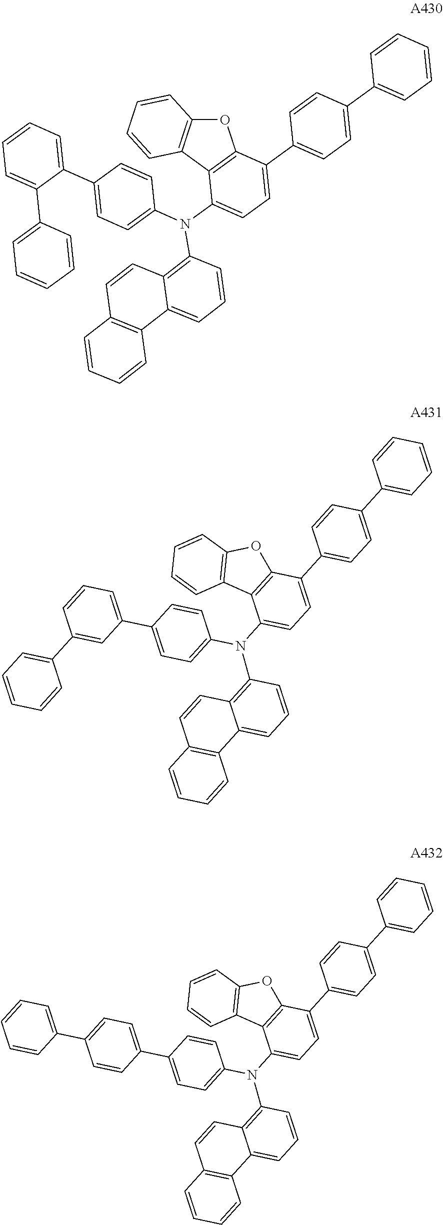 [SMILES: c1ccc(-c2ccc(-c3ccc(N(c4ccc(-c5ccccc5-c5ccccc5)cc4)c4cccc5c4ccc4ccccc45)c4c3oc3ccccc34)cc2)cc1 ; c1ccc(-c2ccc(-c3ccc(N(c4ccc(-c5cccc(-c6ccccc6)c5)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccc(-c6ccccc6)cc5)c5oc6ccccc6c45)cc3)cc2)cc1] 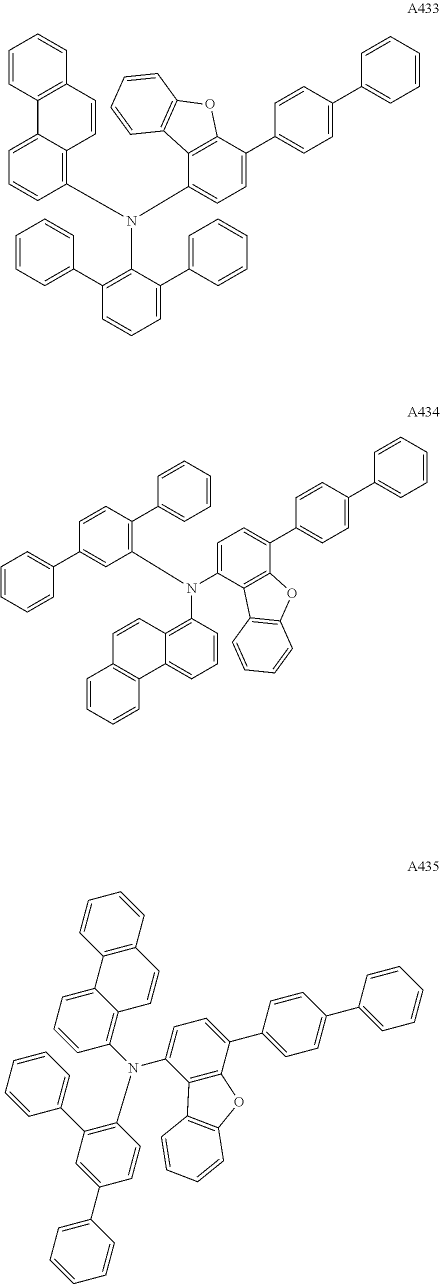 [SMILES: c1ccc(-c2ccc(-c3ccc(N(c4c(-c5ccccc5)cccc4-c4ccccc4)c4cccc5c4ccc4ccccc45)c4c3oc3ccccc34)cc2)cc1 ; c1ccc(-c2ccc(-c3ccc(N(c4cc(-c5ccccc5)ccc4-c4ccccc4)c4cccc5c4ccc4ccccc45)c4c3oc3ccccc34)cc2)cc1 ; c1ccc(-c2ccc(-c3ccc(N(c4ccc(-c5ccccc5)cc4-c4ccccc4)c4cccc5c4ccc4ccccc45)c4c3oc3ccccc34)cc2)cc1] 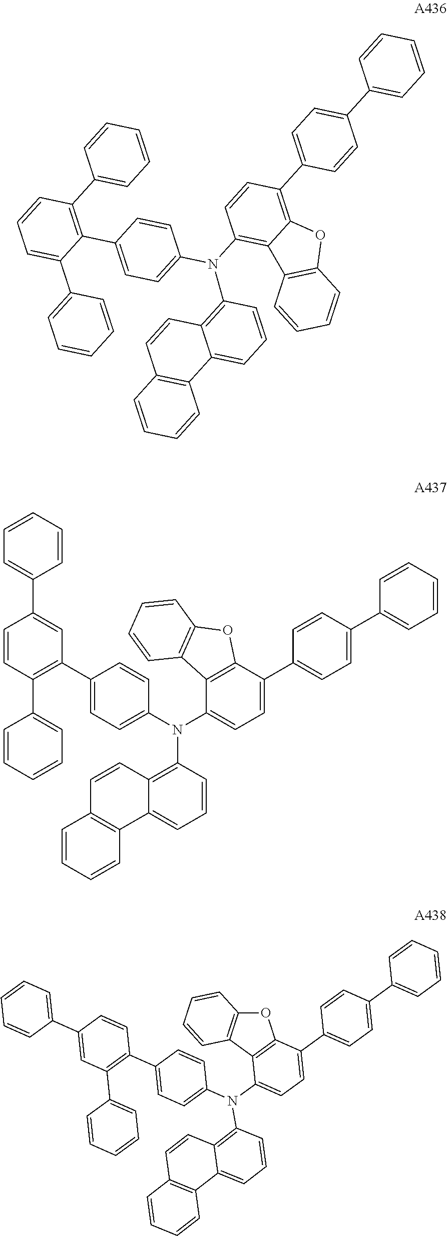 [SMILES: c1ccc(-c2ccc(-c3ccc(N(c4ccc(-c5c(-c6ccccc6)cccc5-c5ccccc5)cc4)c4cccc5c4ccc4ccccc45)c4c3oc3ccccc34)cc2)cc1 ; c1ccc(-c2ccc(-c3ccc(N(c4ccc(-c5cc(-c6ccccc6)ccc5-c5ccccc5)cc4)c4cccc5c4ccc4ccccc45)c4c3oc3ccccc34)cc2)cc1 ; c1ccc(-c2ccc(-c3ccc(N(c4ccc(-c5ccc(-c6ccccc6)cc5-c5ccccc5)cc4)c4cccc5c4ccc4ccccc45)c4c3oc3ccccc34)cc2)cc1] 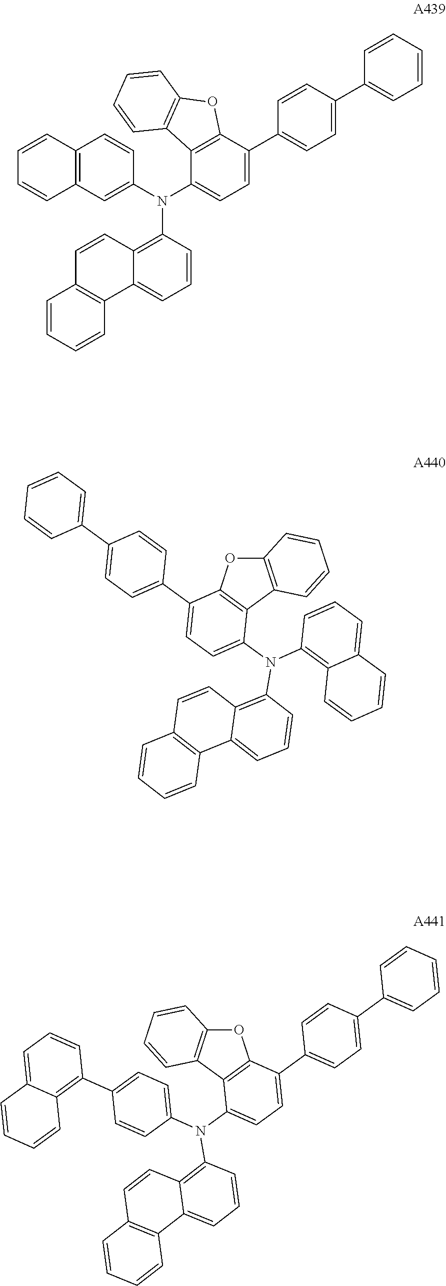 [SMILES: c1ccc(-c2ccc(-c3ccc(N(c4ccc5ccccc5c4)c4cccc5c4ccc4ccccc45)c4c3oc3ccccc34)cc2)cc1 ; c1ccc(-c2ccc(-c3ccc(N(c4cccc5ccccc45)c4cccc5c4ccc4ccccc45)c4c3oc3ccccc34)cc2)cc1 ; c1ccc(-c2ccc(-c3ccc(N(c4ccc(-c5cccc6ccccc56)cc4)c4cccc5c4ccc4ccccc45)c4c3oc3ccccc34)cc2)cc1] 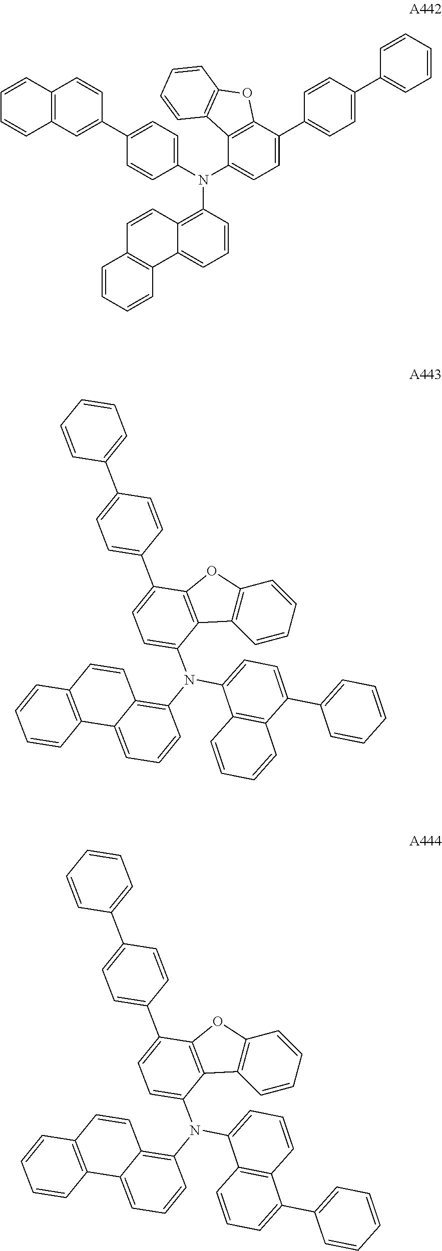 [SMILES: c1ccc(-c2ccc(-c3ccc(N(c4ccc(-c5ccc6ccccc6c5)cc4)c4cccc5c4ccc4ccccc45)c4c3oc3ccccc34)cc2)cc1 ; c1ccc(-c2ccc(-c3ccc(N(c4ccc(-c5ccccc5)c5ccccc45)c4cccc5c4ccc4ccccc45)c4c3oc3ccccc34)cc2)cc1 ; c1ccc(-c2ccc(-c3ccc(N(c4cccc5c(-c6ccccc6)cccc45)c4cccc5c4ccc4ccccc45)c4c3oc3ccccc34)cc2)cc1] 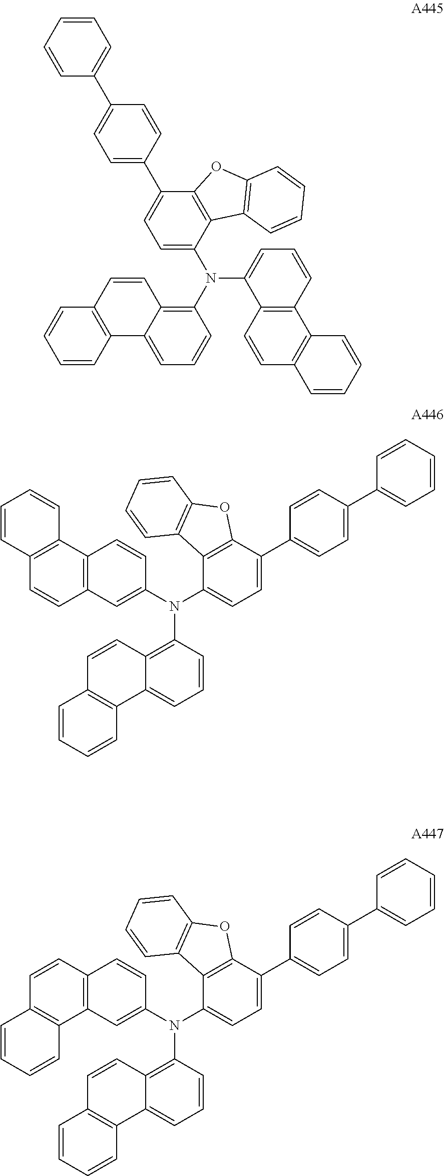 [SMILES: c1ccc(-c2ccc(-c3ccc(N(c4cccc5c4ccc4ccccc45)c4cccc5c4ccc4ccccc45)c4c3oc3ccccc34)cc2)cc1 ; c1ccc(-c2ccc(-c3ccc(N(c4ccc5c(ccc6ccccc65)c4)c4cccc5c4ccc4ccccc45)c4c3oc3ccccc34)cc2)cc1 ; c1ccc(-c2ccc(-c3ccc(N(c4ccc5ccc6ccccc6c5c4)c4cccc5c4ccc4ccccc45)c4c3oc3ccccc34)cc2)cc1] 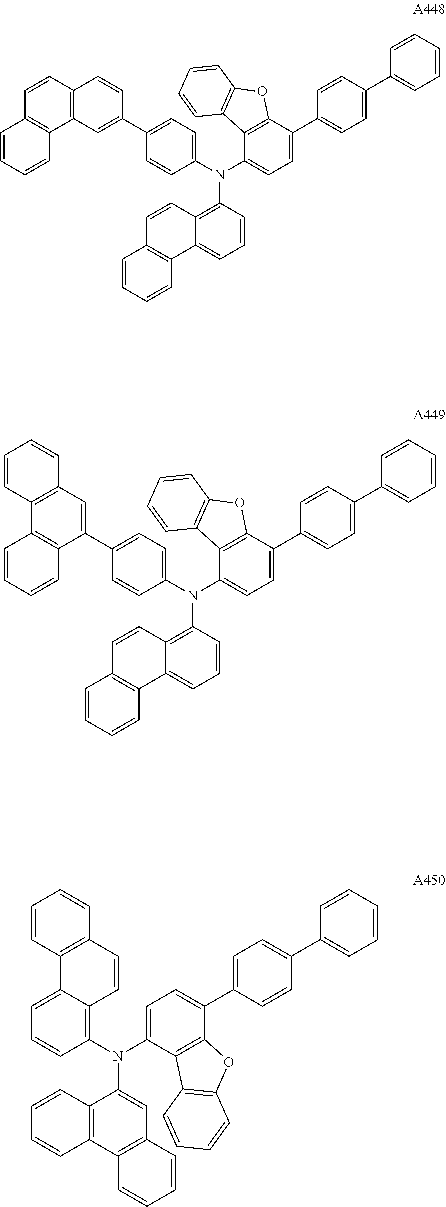 [SMILES: c1ccc(-c2ccc(-c3ccc(N(c4ccc(-c5ccc6ccc7ccccc7c6c5)cc4)c4cccc5c4ccc4ccccc45)c4c3oc3ccccc34)cc2)cc1 ; c1ccc(-c2ccc(-c3ccc(N(c4ccc(-c5cc6ccccc6c6ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4cc5ccccc5c5ccccc45)c4c3oc3ccccc34)cc2)cc1] 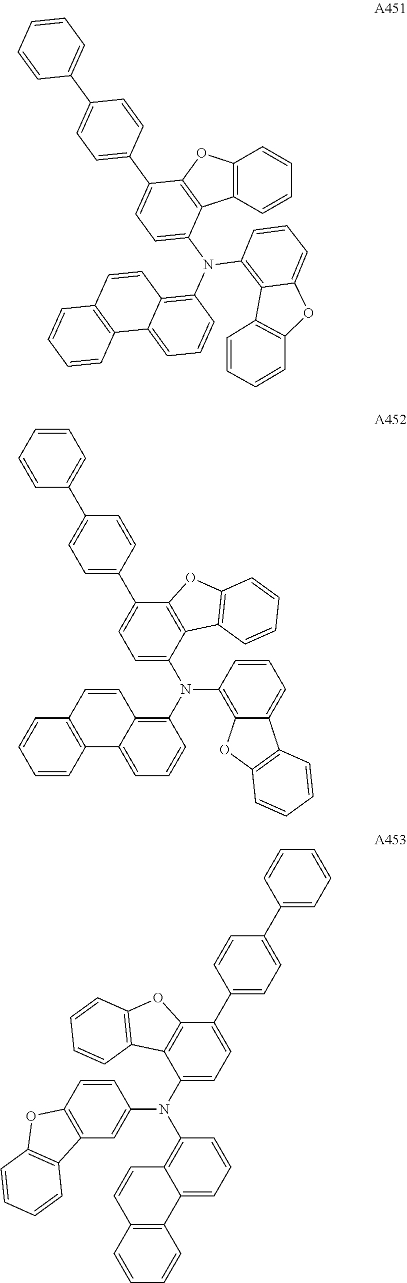 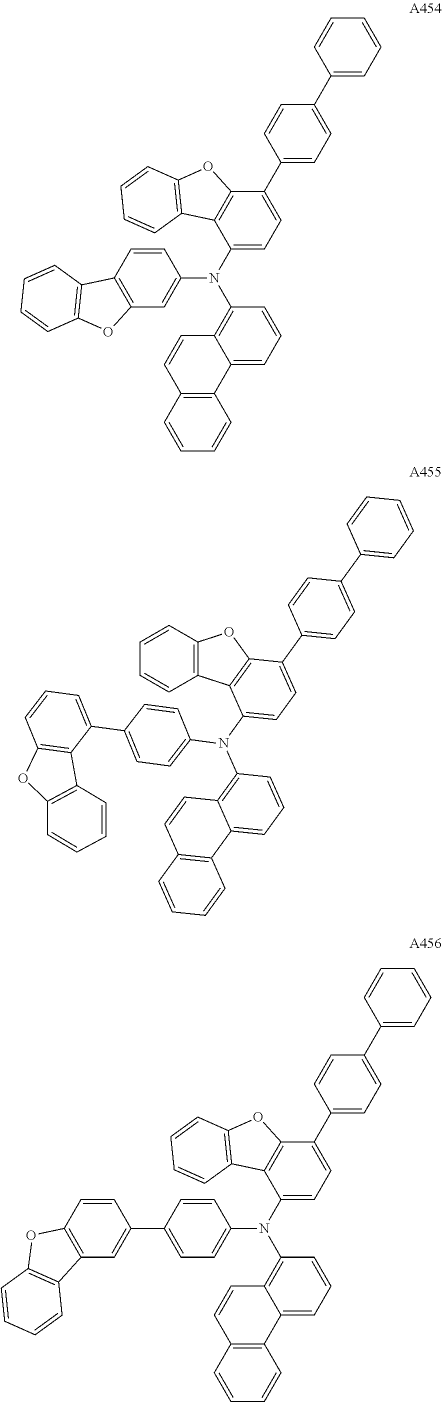 [SMILES: c1ccc(-c2ccc(-c3ccc(N(c4ccc5c(c4)oc4ccccc45)c4cccc5c4ccc4ccccc45)c4c3oc3ccccc34)cc2)cc1 ; c1ccc(-c2ccc(-c3ccc(N(c4ccc(-c5cccc6oc7ccccc7c56)cc4)c4cccc5c4ccc4ccccc45)c4c3oc3ccccc34)cc2)cc1 ; c1ccc(-c2ccc(-c3ccc(N(c4ccc(-c5ccc6oc7ccccc7c6c5)cc4)c4cccc5c4ccc4ccccc45)c4c3oc3ccccc34)cc2)cc1] 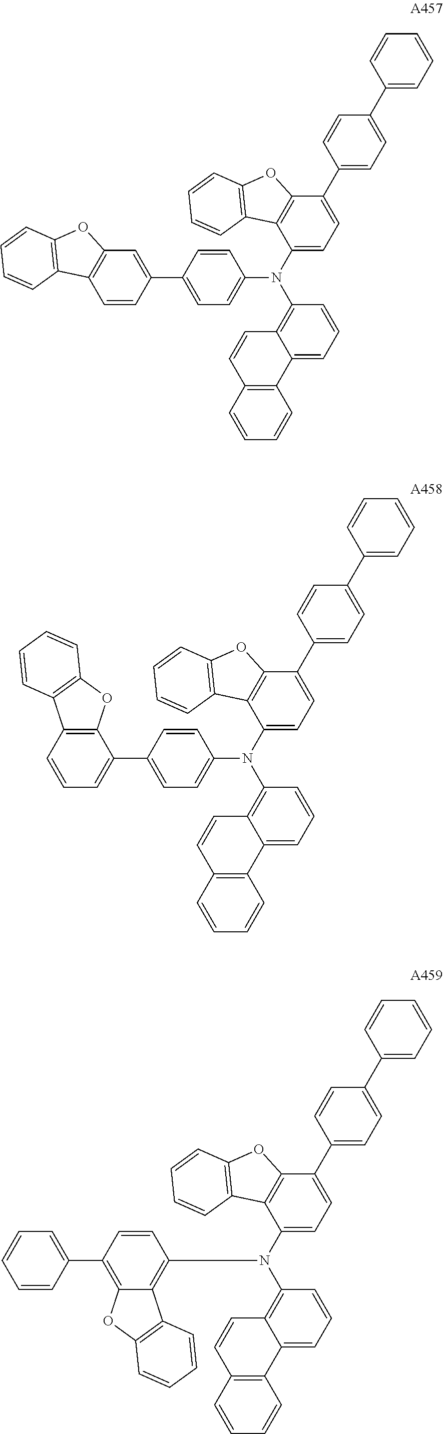 [SMILES: c1ccc(-c2ccc(-c3ccc(N(c4ccc(-c5ccc6c(c5)oc5ccccc56)cc4)c4cccc5c4ccc4ccccc45)c4c3oc3ccccc34)cc2)cc1 ; c1ccc(-c2ccc(-c3ccc(N(c4ccc(-c5cccc6c5oc5ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccccc5)c5oc6ccccc6c45)c4c3oc3ccccc34)cc2)cc1] 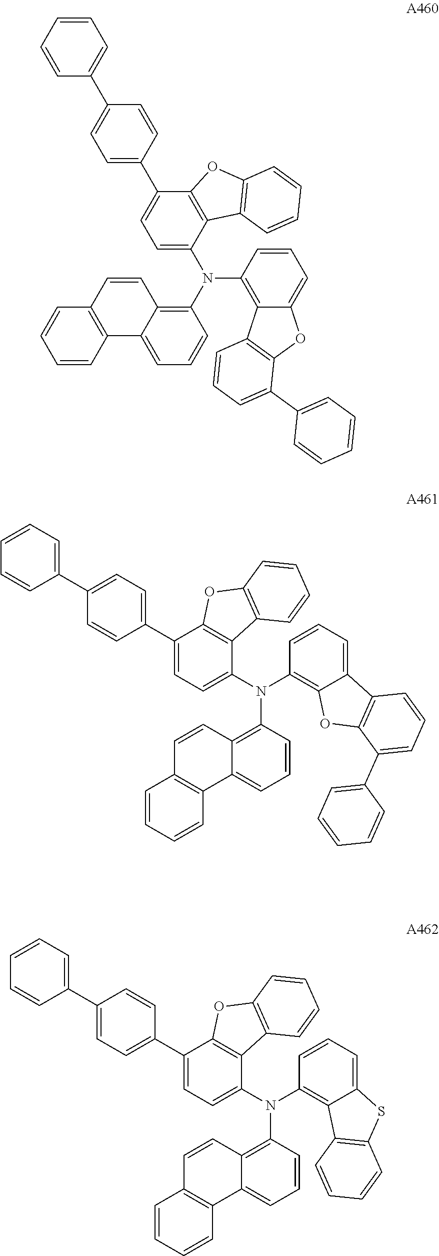 [SMILES: c1ccc(-c2ccc(-c3ccc(N(c4cccc5c4ccc4ccccc45)c4cccc5oc6c(-c7ccccc7)cccc6c45)c4c3oc3ccccc34)cc2)cc1 ; c1ccc(-c2ccc(-c3ccc(N(c4cccc5c4ccc4ccccc45)c4cccc5c4oc4c(-c6ccccc6)cccc45)c4c3oc3ccccc34)cc2)cc1 ; c1ccc(-c2ccc(-c3ccc(N(c4cccc5c4ccc4ccccc45)c4cccc5sc6ccccc6c45)c4c3oc3ccccc34)cc2)cc1] 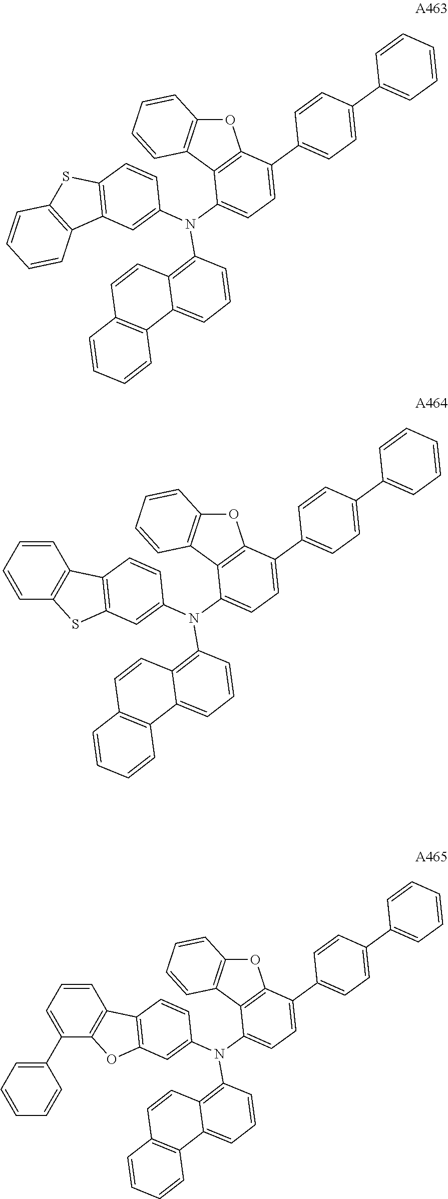 [SMILES: c1ccc(-c2ccc(-c3ccc(N(c4ccc5sc6ccccc6c5c4)c4cccc5c4ccc4ccccc45)c4c3oc3ccccc34)cc2)cc1 ; c1ccc(-c2ccc(-c3ccc(N(c4ccc5c(c4)sc4ccccc45)c4cccc5c4ccc4ccccc45)c4c3oc3ccccc34)cc2)cc1 ; c1ccc(-c2ccc(-c3ccc(N(c4ccc5c(c4)oc4c(-c6ccccc6)cccc45)c4cccc5c4ccc4ccccc45)c4c3oc3ccccc34)cc2)cc1] 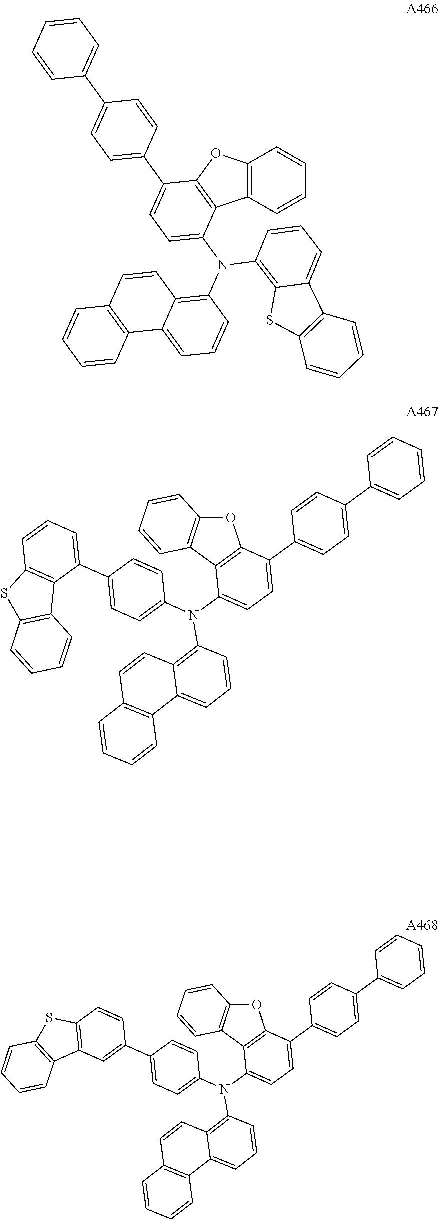 [SMILES: c1ccc(-c2ccc(-c3ccc(N(c4cccc5c4ccc4ccccc45)c4cccc5c4sc4ccccc45)c4c3oc3ccccc34)cc2)cc1 ; c1ccc(-c2ccc(-c3ccc(N(c4ccc(-c5cccc6sc7ccccc7c56)cc4)c4cccc5c4ccc4ccccc45)c4c3oc3ccccc34)cc2)cc1 ; c1ccc(-c2ccc(-c3ccc(N(c4ccc(-c5ccc6sc7ccccc7c6c5)cc4)c4cccc5c4ccc4ccccc45)c4c3oc3ccccc34)cc2)cc1] 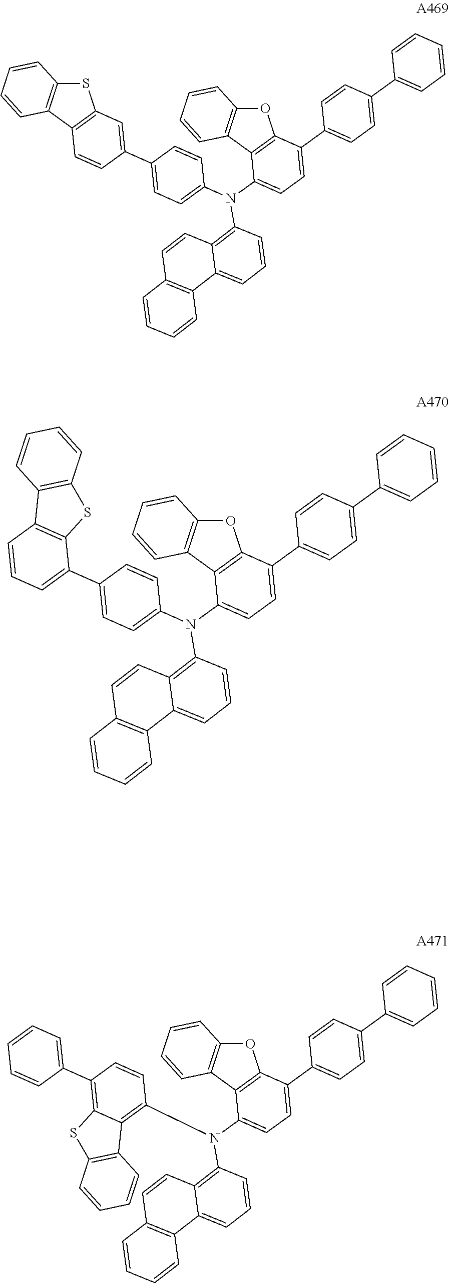 [SMILES: c1ccc(-c2ccc(-c3ccc(N(c4ccc(-c5ccc6c(c5)sc5ccccc56)cc4)c4cccc5c4ccc4ccccc45)c4c3oc3ccccc34)cc2)cc1 ; c1ccc(-c2ccc(-c3ccc(N(c4ccc(-c5cccc6c5sc5ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccccc5)c5sc6ccccc6c45)c4c3oc3ccccc34)cc2)cc1] 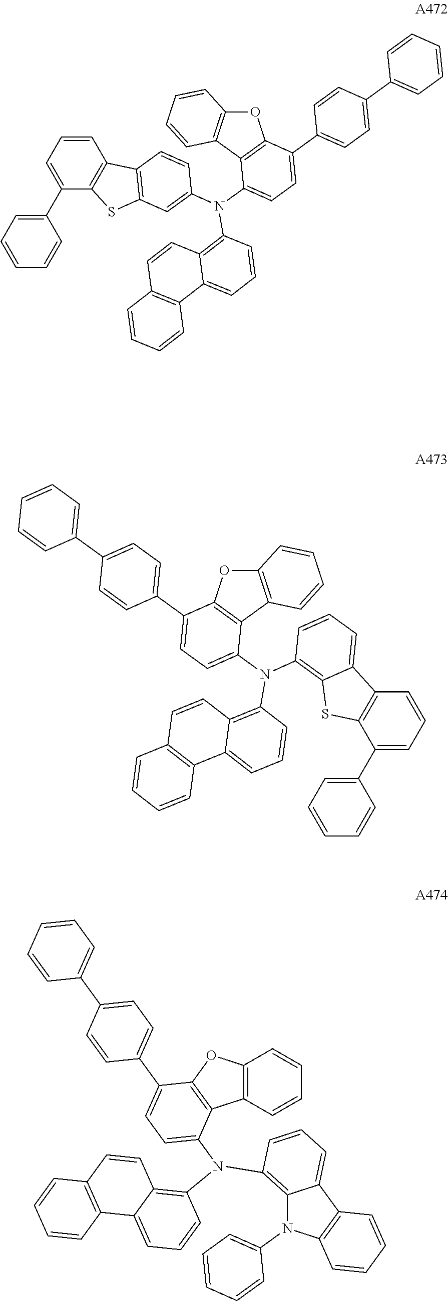 [SMILES: c1ccc(-c2ccc(-c3ccc(N(c4ccc5c(c4)sc4c(-c6ccccc6)cccc45)c4cccc5c4ccc4ccccc45)c4c3oc3ccccc34)cc2)cc1 ; c1ccc(-c2ccc(-c3ccc(N(c4cccc5c4ccc4ccccc45)c4cccc5c4sc4c(-c6ccccc6)cccc45)c4c3oc3ccccc34)cc2)cc1 ; c1ccc(-c2ccc(-c3ccc(N(c4cccc5c4ccc4ccccc45)c4cccc5c6ccccc6n(-c6ccccc6)c45)c4c3oc3ccccc34)cc2)cc1] 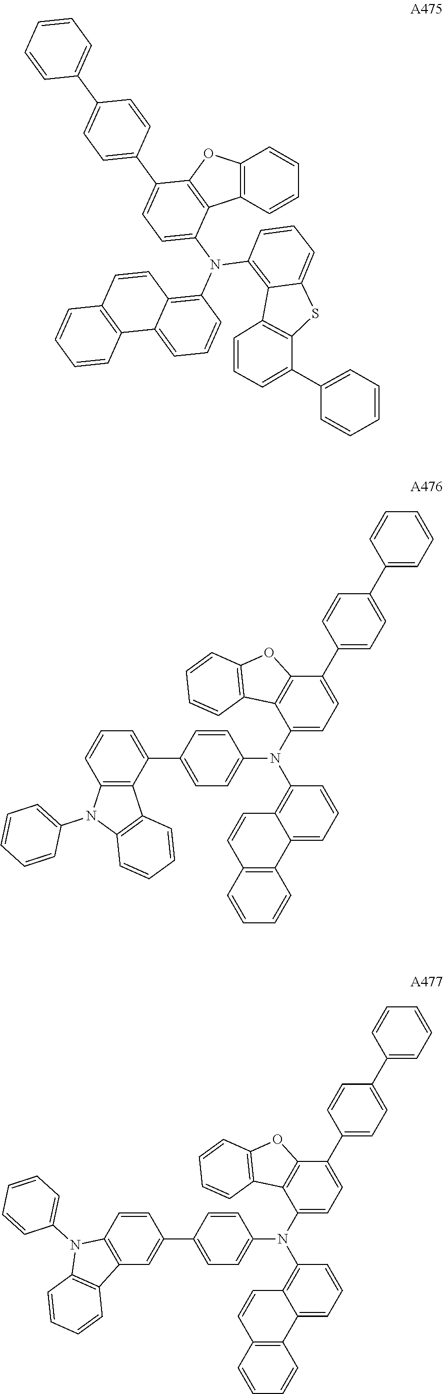 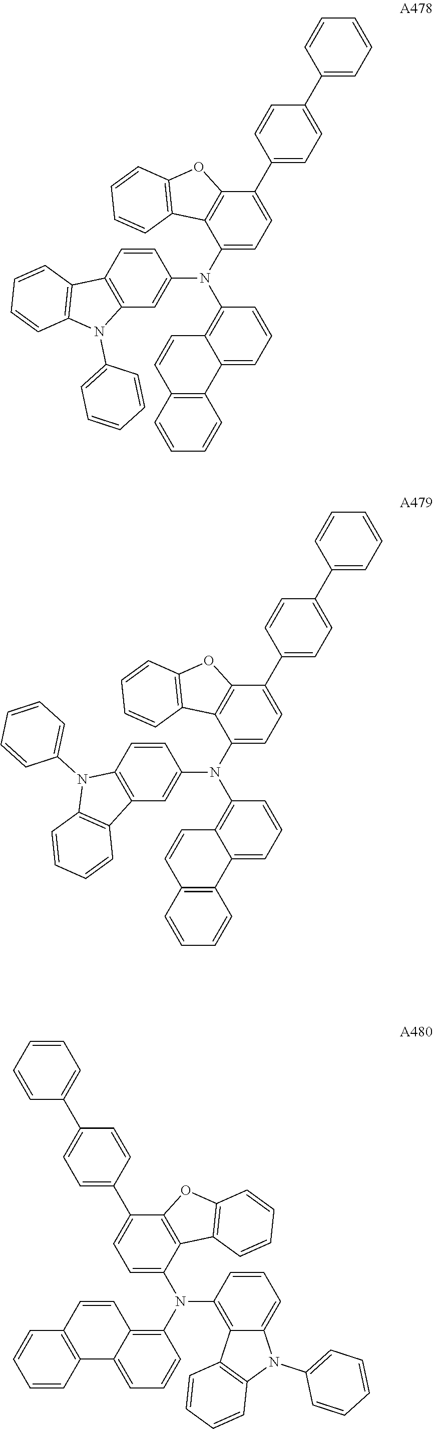 [SMILES: c1ccc(-c2ccc(-c3ccc(N(c4ccc5c6ccccc6n(-c6ccccc6)c5c4)c4cccc5c4ccc4ccccc45)c4c3oc3ccccc34)cc2)cc1 ; c1ccc(-c2ccc(-c3ccc(N(c4ccc5c(c4)c4ccccc4n5-c4ccccc4)c4cccc5c4ccc4ccccc45)c4c3oc3ccccc34)cc2)cc1 ; c1ccc(-c2ccc(-c3ccc(N(c4cccc5c4ccc4ccccc45)c4cccc5c4c4ccccc4n5-c4ccccc4)c4c3oc3ccccc34)cc2)cc1] 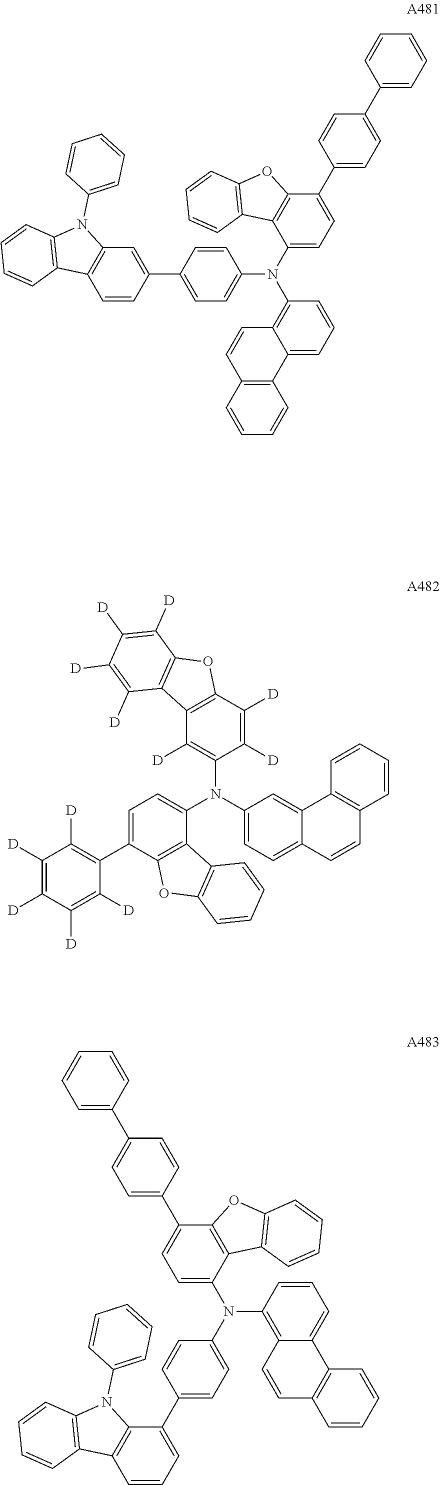 [SMILES: c1ccc(-c2ccc(-c3ccc(N(c4ccc(-c5ccc6c7ccccc7n(-c7ccccc7)c6c5)cc4)c4cccc5c4ccc4ccccc45)c4c3oc3ccccc34)cc2)cc1 ; [2H]c1c([2H])c([2H])c(-c2ccc(N(c3ccc4ccc5ccccc5c4c3)c3c([2H])c([2H])c4oc5c([2H])c([2H])c([2H])c([2H])c5c4c3[2H])c3c2oc2ccccc23)c([2H])c1[2H] ; c1ccc(-c2ccc(-c3ccc(N(c4ccc(-c5cccc6c7ccccc7n(-c7ccccc7)c56)cc4)c4cccc5c4ccc4ccccc45)c4c3oc3ccccc34)cc2)cc1] 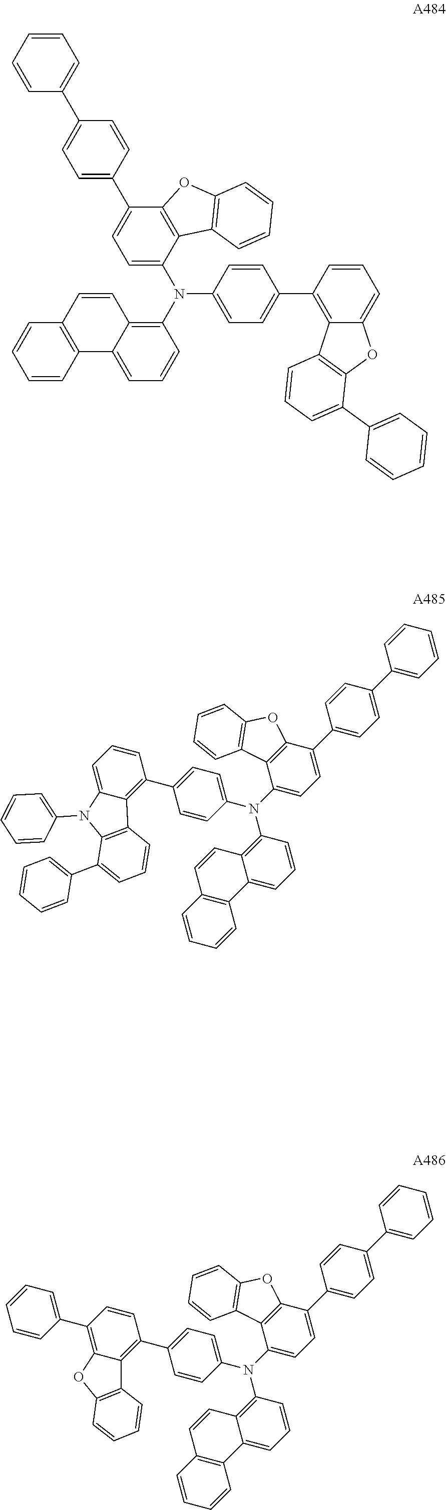 [SMILES: c1ccc(-c2ccc(-c3ccc(N(c4ccc(-c5cccc6oc7c(-c8ccccc8)cccc7c56)cc4)c4cccc5c4ccc4ccccc45)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4cccc5c4ccc4ccccc45)c4c3oc3ccccc34)cc2)cc1 ; c1ccc(-c2ccc(-c3ccc(N(c4ccc(-c5ccc(-c6ccccc6)c6oc7ccccc7c56)cc4)c4cccc5c4ccc4ccccc45)c4c3oc3ccccc34)cc2)cc1] 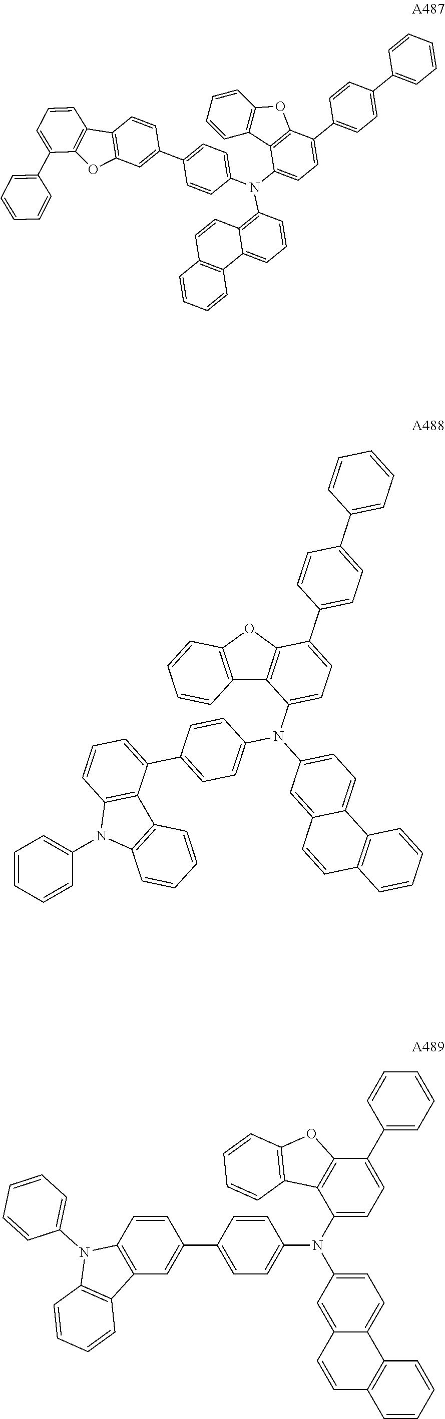 [SMILES: c1ccc(-c2ccc(-c3ccc(N(c4ccc(-c5ccc6c(c5)oc5c(-c7ccccc7)cccc56)cc4)c4cccc5c4ccc4ccccc45)c4c3oc3ccccc34)cc2)cc1 ; c1ccc(-c2ccc(-c3ccc(N(c4ccc(-c5cccc6c5c5ccccc5n6-c5ccccc5)cc4)c4ccc5c(ccc6ccccc65)c4)c4c3oc3ccccc34)cc2)cc1 ; c1ccc(-c2ccc(N(c3ccc(-c4ccc5c(c4)c4ccccc4n5-c4ccccc4)cc3)c3ccc4c(ccc5ccccc54)c3)c3c2oc2ccccc23)cc1] 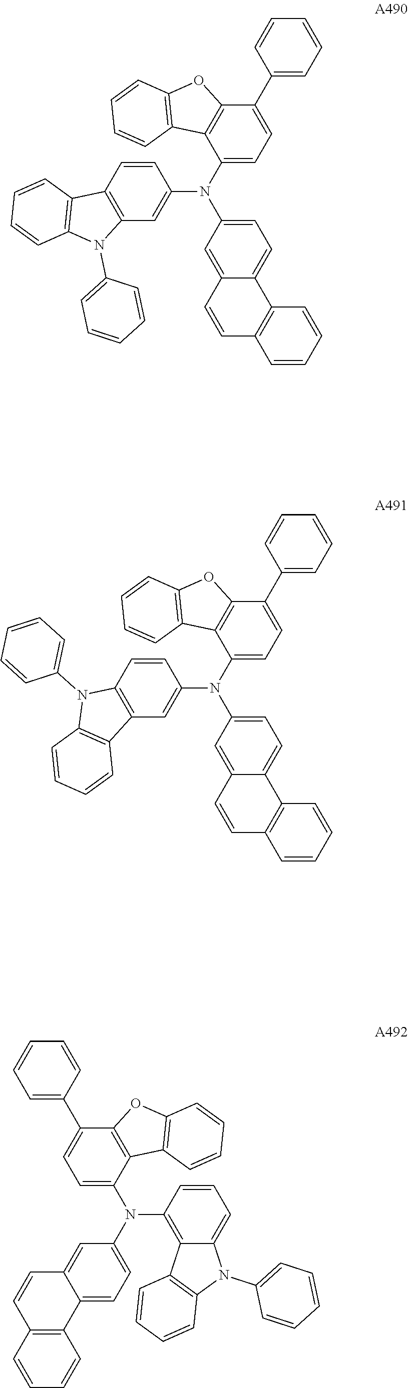 [SMILES: c1ccc(-c2ccc(N(c3ccc4c(ccc5ccccc54)c3)c3ccc4c5ccccc5n(-c5ccccc5)c4c3)c3c2oc2ccccc23)cc1 ; c1ccc(-c2ccc(N(c3ccc4c(ccc5ccccc54)c3)c3ccc4c(c3)c3ccccc3n4-c3ccccc3)c3c2oc2ccccc23)cc1 ; c1ccc(-c2ccc(N(c3ccc4c(ccc5ccccc54)c3)c3cccc4c3c3ccccc3n4-c3ccccc3)c3c2oc2ccccc23)cc1] 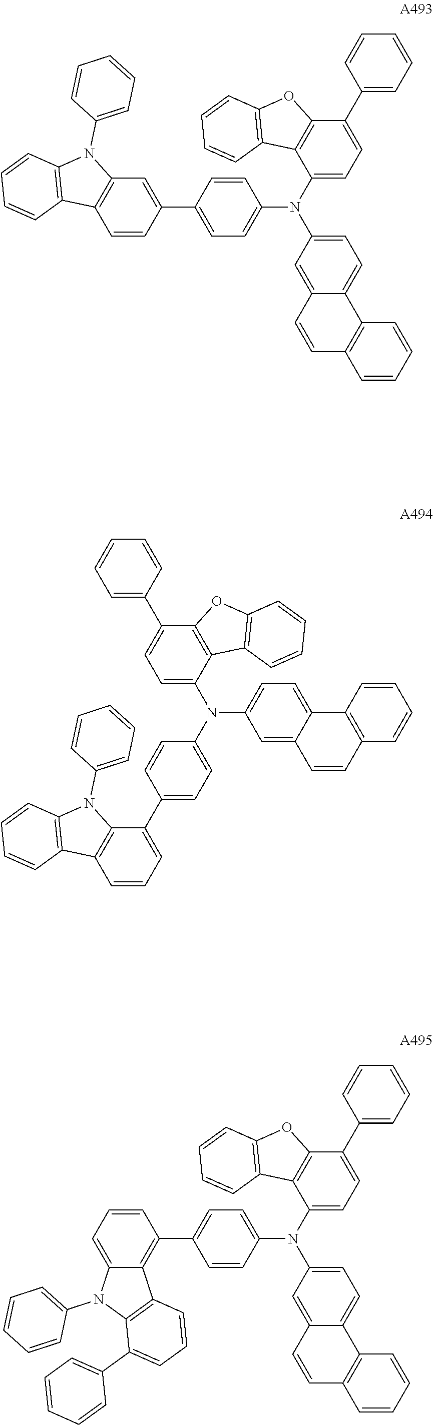 [SMILES: c1ccc(-c2ccc(N(c3ccc(-c4ccc5c6ccccc6n(-c6ccccc6)c5c4)cc3)c3ccc4c(ccc5ccccc54)c3)c3c2oc2ccccc23)cc1 ; c1ccc(-c2ccc(N(c3ccc(-c4cccc5c6ccccc6n(-c6ccccc6)c45)cc3)c3ccc4c(ccc5ccccc54)c3)c3c2oc2ccccc23)cc1 ; c1ccc(-c2ccc(N(c3ccc(-c4cccc5c4c4cccc(-c6ccccc6)c4n5-c4ccccc4)cc3)c3ccc4c(ccc5ccccc54)c3)c3c2oc2ccccc23)cc1] 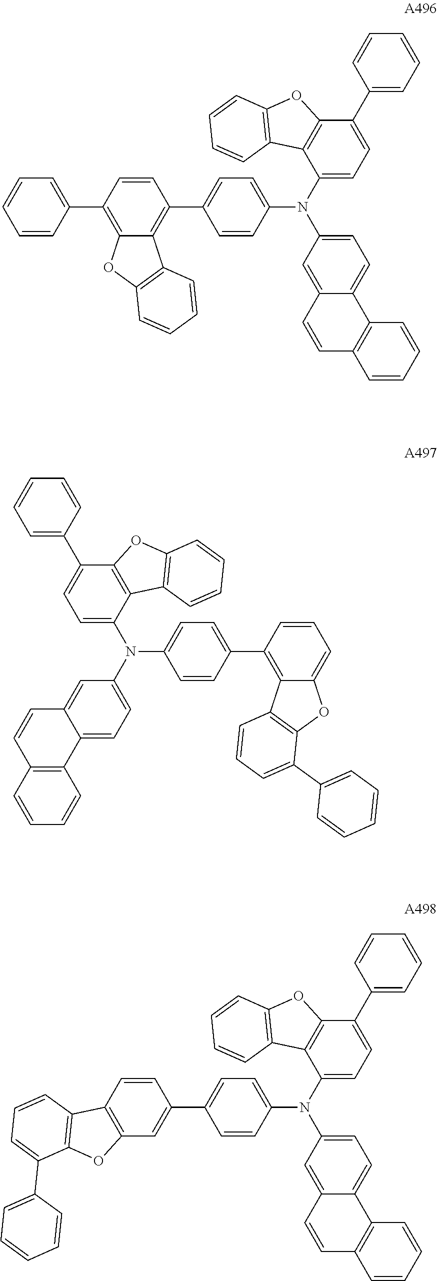 [SMILES: c1ccc(-c2ccc(-c3ccc(N(c4ccc5c(ccc6ccccc65)c4)c4ccc(-c5ccccc5)c5oc6ccccc6c45)cc3)c3c2oc2ccccc23)cc1 ; c1ccc(-c2cccc3c2oc2cccc(-c4ccc(N(c5ccc6c(ccc7ccccc76)c5)c5ccc(-c6ccccc6)c6oc7ccccc7c56)cc4)c23)cc1 ; c1ccc(-c2cccc3c2oc2cc(-c4ccc(N(c5ccc6c(ccc7ccccc76)c5)c5ccc(-c6ccccc6)c6oc7ccccc7c56)cc4)ccc23)cc1] 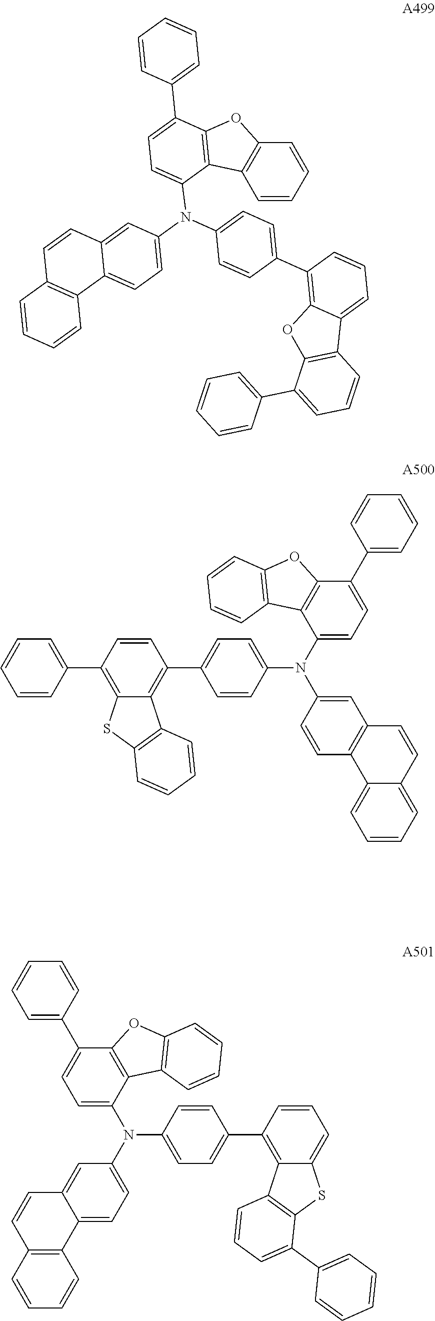 [SMILES: c1ccc(-c2cccc3c2oc2c(-c4ccc(N(c5ccc6c(ccc7ccccc76)c5)c5ccc(-c6ccccc6)c6oc7ccccc7c56)cc4)cccc23)cc1 ; c1ccc(-c2ccc(N(c3ccc(-c4ccc(-c5ccccc5)c5sc6ccccc6c45)cc3)c3ccc4c(ccc5ccccc54)c3)c3c2oc2ccccc23)cc1 ; c1ccc(-c2ccc(N(c3ccc(-c4cccc5sc6c(-c7ccccc7)cccc6c45)cc3)c3ccc4c(ccc5ccccc54)c3)c3c2oc2ccccc23)cc1] 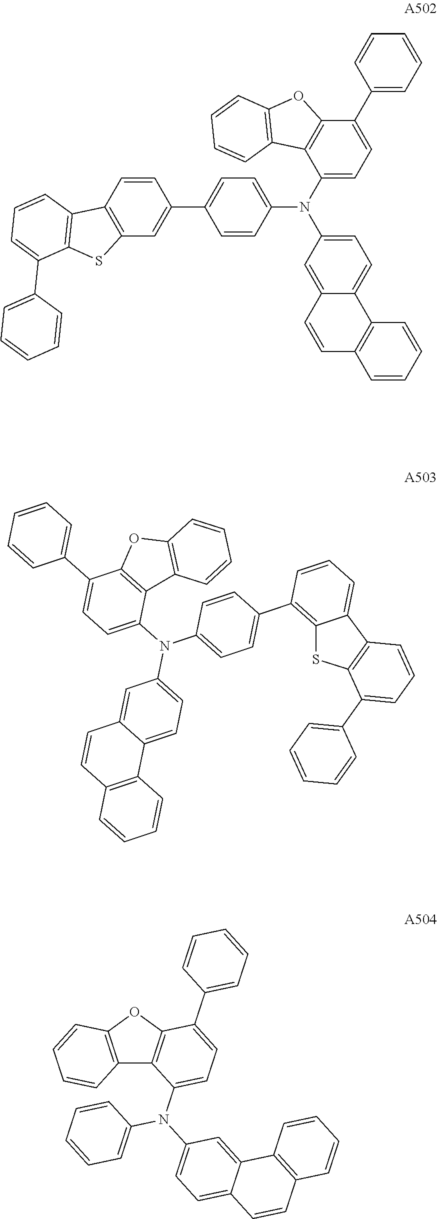 [SMILES: c1ccc(-c2ccc(N(c3ccc(-c4ccc5c(c4)sc4c(-c6ccccc6)cccc45)cc3)c3ccc4c(ccc5ccccc54)c3)c3c2oc2ccccc23)cc1 ; c1ccc(-c2ccc(N(c3ccc(-c4cccc5c4sc4c(-c6ccccc6)cccc45)cc3)c3ccc4c(ccc5ccccc54)c3)c3c2oc2ccccc23)cc1 ; c1ccc(-c2ccc(N(c3ccccc3)c3ccc4ccc5ccccc5c4c3)c3c2oc2ccccc23)cc1] 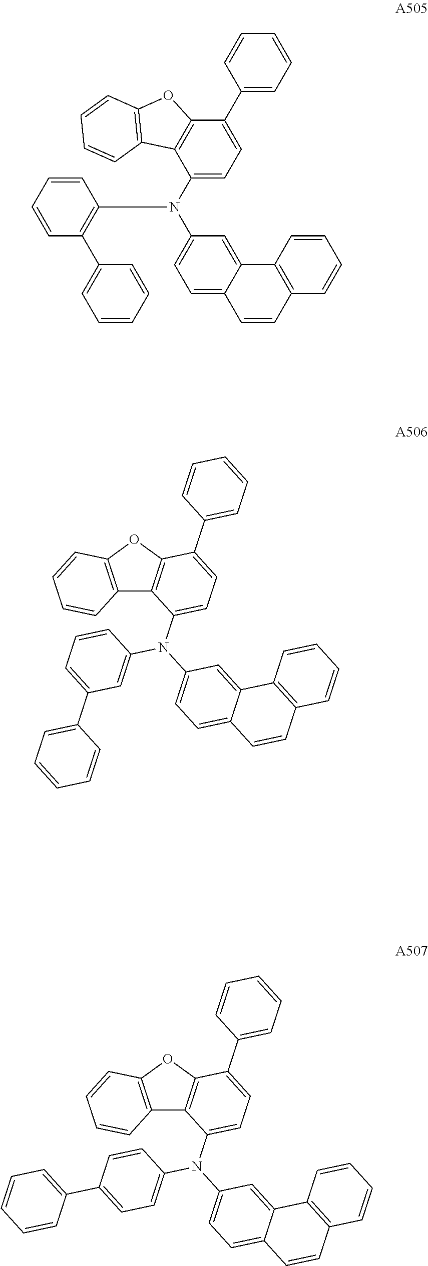 [SMILES: c1ccc(-c2ccccc2N(c2ccc3ccc4ccccc4c3c2)c2ccc(-c3ccccc3)c3oc4ccccc4c23)cc1 ; c1ccc(-c2cccc(N(c3ccc4ccc5ccccc5c4c3)c3ccc(-c4ccccc4)c4oc5ccccc5c34)c2)cc1 ; c1ccc(-c2ccc(N(c3ccc4ccc5ccccc5c4c3)c3ccc(-c4ccccc4)c4oc5ccccc5c34)cc2)cc1] 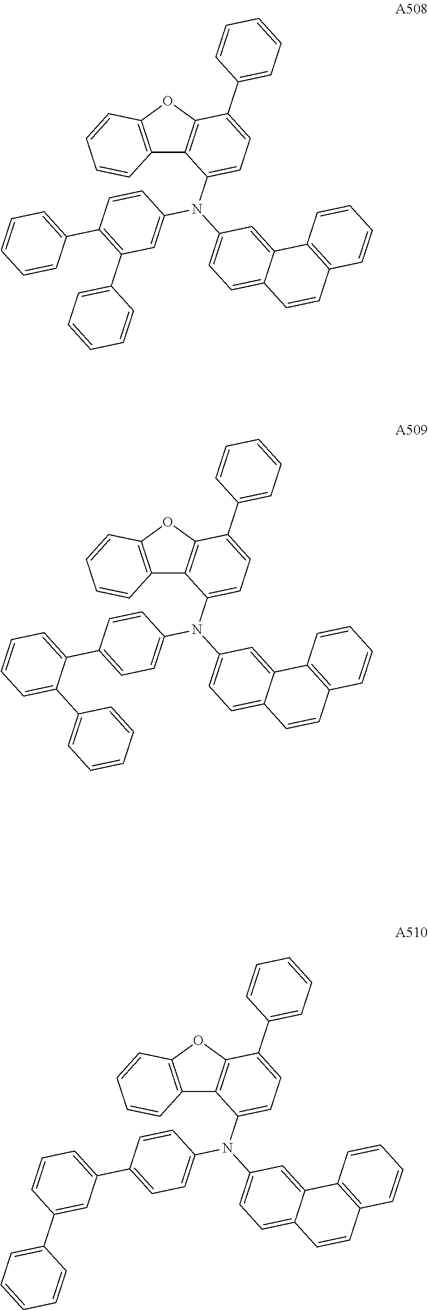 [SMILES: c1ccc(-c2ccc(N(c3ccc4ccc5ccccc5c4c3)c3ccc(-c4ccccc4)c4oc5ccccc5c34)cc2-c2ccccc2)cc1 ; c1ccc(-c2ccccc2-c2ccc(N(c3ccc4ccc5ccccc5c4c3)c3ccc(-c4ccccc4)c4oc5ccccc5c34)cc2)cc1 ; c1ccc(-c2cccc(-c3ccc(N(c4ccc5ccc6ccccc6c5c4)c4ccc(-c5ccccc5)c5oc6ccccc6c45)cc3)c2)cc1] 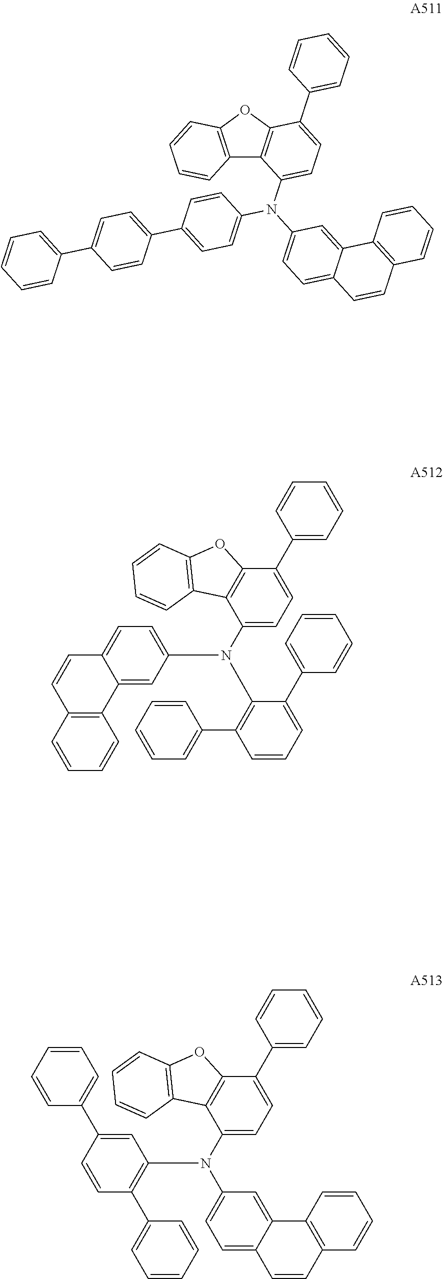 [SMILES: c1ccc(-c2ccc(-c3ccc(N(c4ccc5ccc6ccccc6c5c4)c4ccc(-c5ccccc5)c5oc6ccccc6c45)cc3)cc2)cc1 ; c1ccc(-c2cccc(-c3ccccc3)c2N(c2ccc3ccc4ccccc4c3c2)c2ccc(-c3ccccc3)c3oc4ccccc4c23)cc1 ; c1ccc(-c2ccc(-c3ccccc3)c(N(c3ccc4ccc5ccccc5c4c3)c3ccc(-c4ccccc4)c4oc5ccccc5c34)c2)cc1] 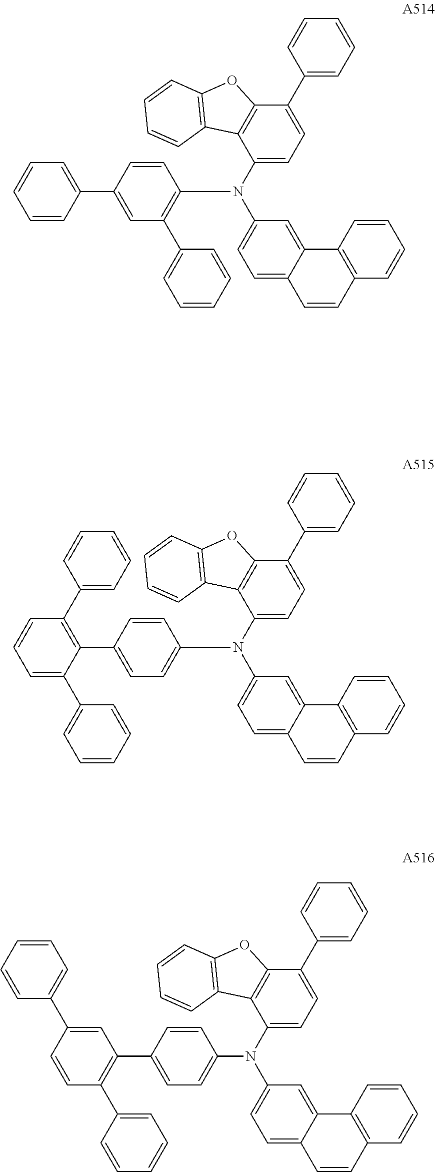 [SMILES: c1ccc(-c2ccc(N(c3ccc4ccc5ccccc5c4c3)c3ccc(-c4ccccc4)c4oc5ccccc5c34)c(-c3ccccc3)c2)cc1 ; c1ccc(-c2cccc(-c3ccccc3)c2-c2ccc(N(c3ccc4ccc5ccccc5c4c3)c3ccc(-c4ccccc4)c4oc5ccccc5c34)cc2)cc1 ; c1ccc(-c2ccc(-c3ccccc3)c(-c3ccc(N(c4ccc5ccc6ccccc6c5c4)c4ccc(-c5ccccc5)c5oc6ccccc6c45)cc3)c2)cc1] 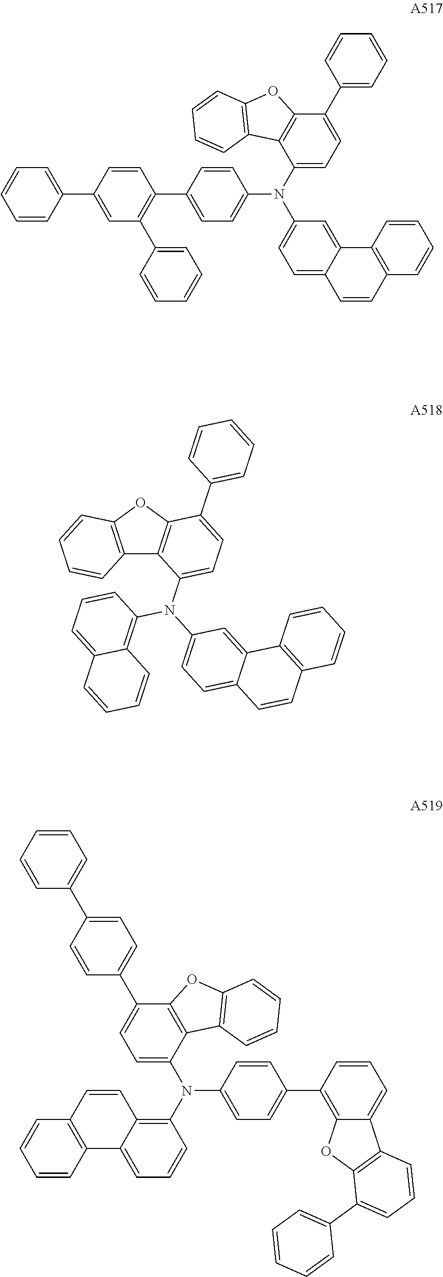 [SMILES: c1ccc(-c2ccc(-c3ccc(N(c4ccc5ccc6ccccc6c5c4)c4ccc(-c5ccccc5)c5oc6ccccc6c45)cc3)c(-c3ccccc3)c2)cc1 ; c1ccc(-c2ccc(N(c3ccc4ccc5ccccc5c4c3)c3cccc4ccccc34)c3c2oc2ccccc23)cc1 ; c1ccc(-c2ccc(-c3ccc(N(c4ccc(-c5cccc6c5oc5c(-c7ccccc7)cccc56)cc4)c4cccc5c4ccc4ccccc45)c4c3oc3ccccc34)cc2)cc1] 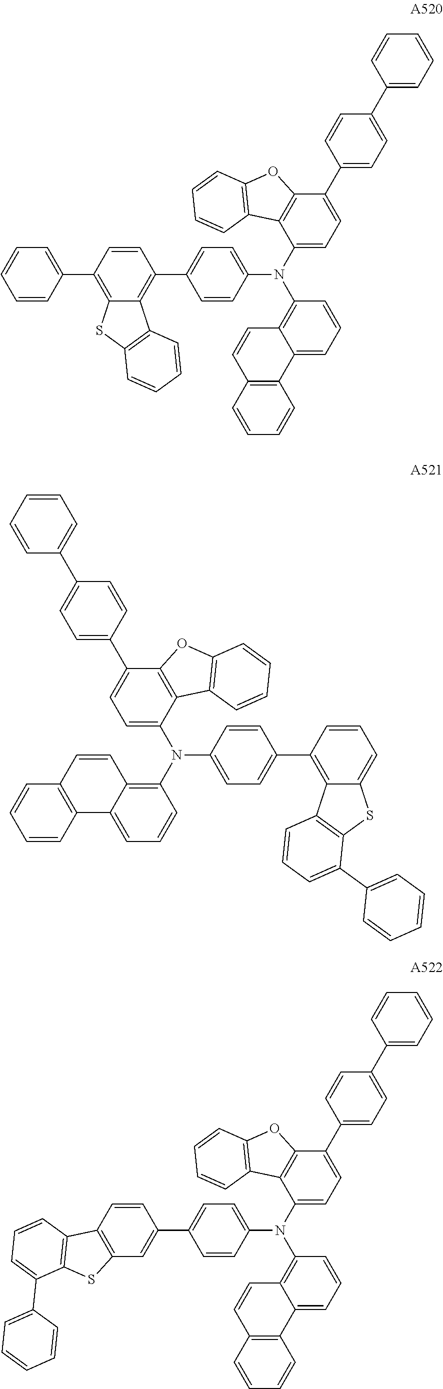 [SMILES: c1ccc(-c2ccc(-c3ccc(N(c4ccc(-c5ccc(-c6ccccc6)c6sc7ccccc7c56)cc4)c4cccc5c4ccc4ccccc45)c4c3oc3ccccc34)cc2)cc1 ; c1ccc(-c2ccc(-c3ccc(N(c4ccc(-c5cccc6sc7c(-c8ccccc8)cccc7c56)cc4)c4cccc5c4ccc4ccccc45)c4c3oc3ccccc34)cc2)cc1 ; c1ccc(-c2ccc(-c3ccc(N(c4ccc(-c5ccc6c(c5)sc5c(-c7ccccc7)cccc56)cc4)c4cccc5c4ccc4ccccc45)c4c3oc3ccccc34)cc2)cc1] 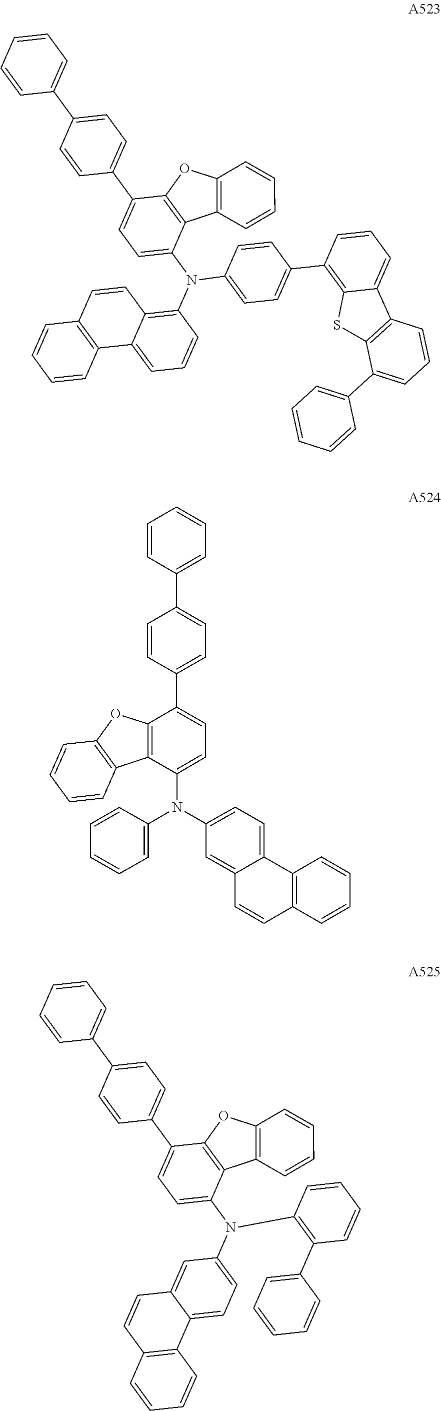 [SMILES: c1ccc(-c2ccc(-c3ccc(N(c4ccc(-c5cccc6c5sc5c(-c7ccccc7)cccc56)cc4)c4cccc5c4ccc4ccccc45)c4c3oc3ccccc34)cc2)cc1 ; c1ccc(-c2ccc(-c3ccc(N(c4ccccc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccccc4-c4ccccc4)c4c3oc3ccccc34)cc2)cc1] 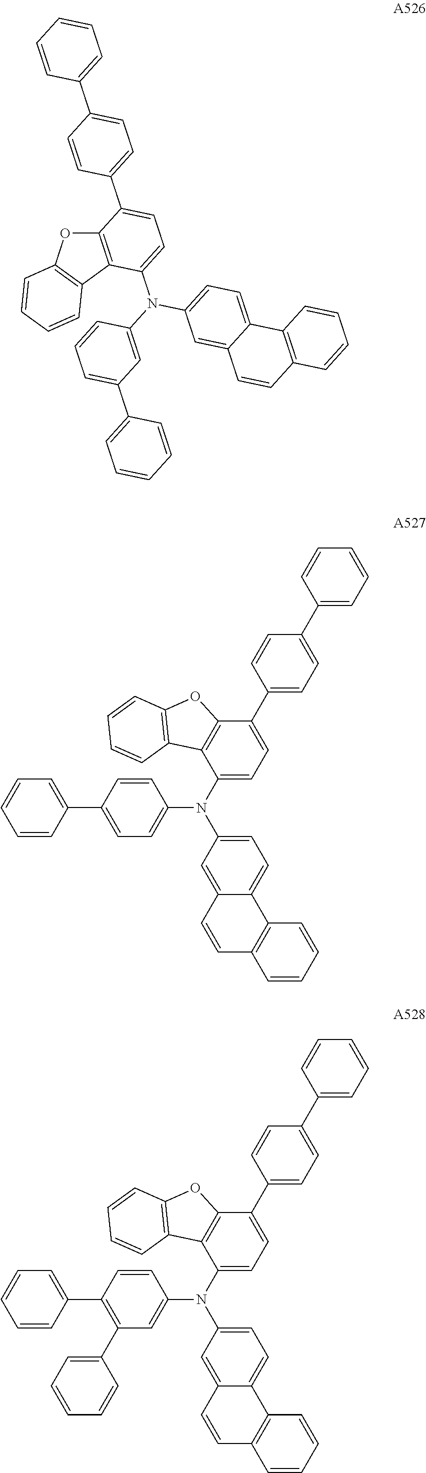 [SMILES: c1ccc(-c2ccc(-c3ccc(N(c4cccc(-c5ccccc5)c4)c4ccc5c(ccc6ccccc65)c4)c4c3oc3ccccc34)cc2)cc1 ; c1ccc(-c2ccc(-c3ccc(N(c4ccc(-c5ccccc5)cc4)c4ccc5c(ccc6ccccc65)c4)c4c3oc3ccccc34)cc2)cc1 ; c1ccc(-c2ccc(-c3ccc(N(c4ccc(-c5ccccc5)c(-c5ccccc5)c4)c4ccc5c(ccc6ccccc65)c4)c4c3oc3ccccc34)cc2)cc1] 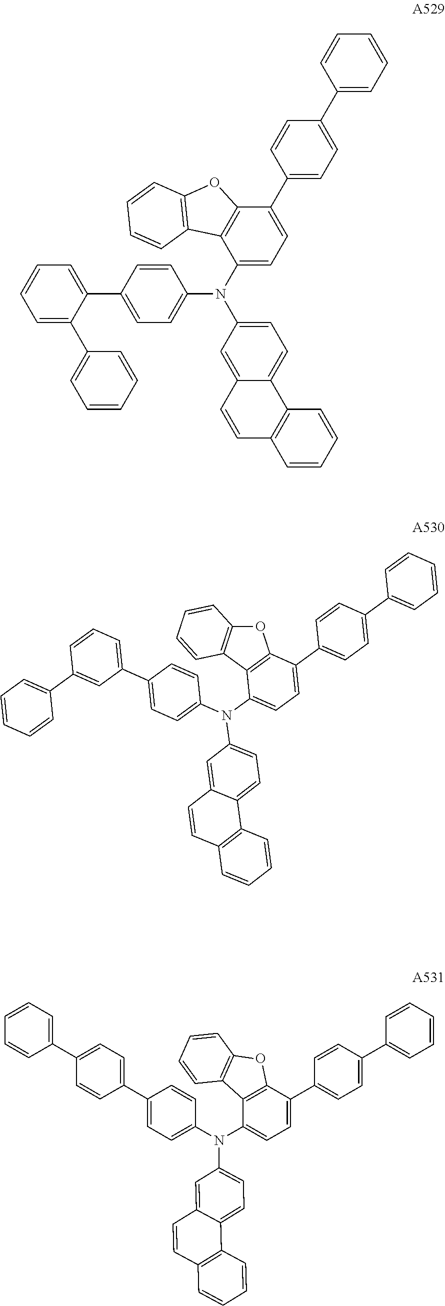 [SMILES: c1ccc(-c2ccc(-c3ccc(N(c4ccc(-c5ccccc5-c5ccccc5)cc4)c4ccc5c(ccc6ccccc65)c4)c4c3oc3ccccc34)cc2)cc1 ; c1ccc(-c2ccc(-c3ccc(N(c4ccc(-c5cccc(-c6ccccc6)c5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccc(-c6ccccc6)cc5)c5oc6ccccc6c45)cc3)cc2)cc1] 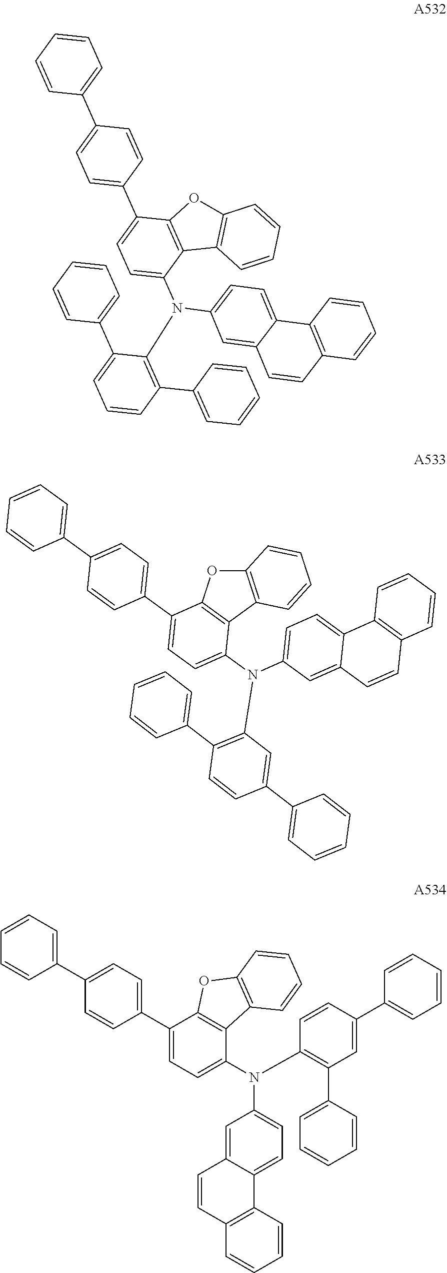 [SMILES: c1ccc(-c2ccc(-c3ccc(N(c4ccc5c(ccc6ccccc65)c4)c4c(-c5ccccc5)cccc4-c4ccccc4)c4c3oc3ccccc34)cc2)cc1 ; c1ccc(-c2ccc(-c3ccc(N(c4ccc5c(ccc6ccccc65)c4)c4cc(-c5ccccc5)ccc4-c4ccccc4)c4c3oc3ccccc34)cc2)cc1 ; c1ccc(-c2ccc(-c3ccc(N(c4ccc5c(ccc6ccccc65)c4)c4ccc(-c5ccccc5)cc4-c4ccccc4)c4c3oc3ccccc34)cc2)cc1] 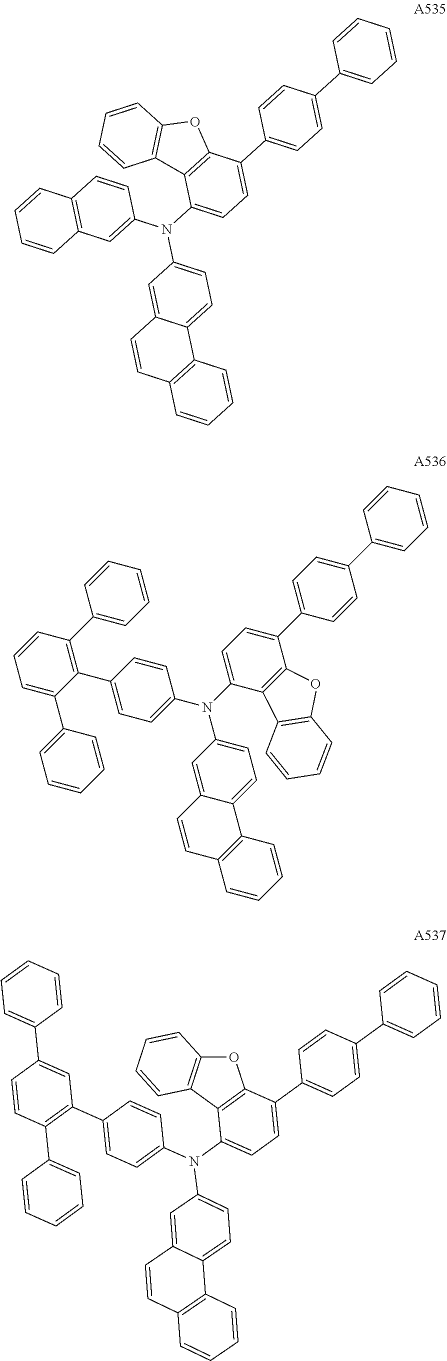 [SMILES: c1ccc(-c2ccc(-c3ccc(N(c4ccc5ccccc5c4)c4ccc5c(ccc6ccccc65)c4)c4c3oc3ccccc34)cc2)cc1 ; c1ccc(-c2ccc(-c3ccc(N(c4ccc(-c5c(-c6ccccc6)cccc5-c5ccccc5)cc4)c4ccc5c(ccc6ccccc65)c4)c4c3oc3ccccc34)cc2)cc1 ; c1ccc(-c2ccc(-c3ccc(N(c4ccc(-c5cc(-c6ccccc6)ccc5-c5ccccc5)cc4)c4ccc5c(ccc6ccccc65)c4)c4c3oc3ccccc34)cc2)cc1] 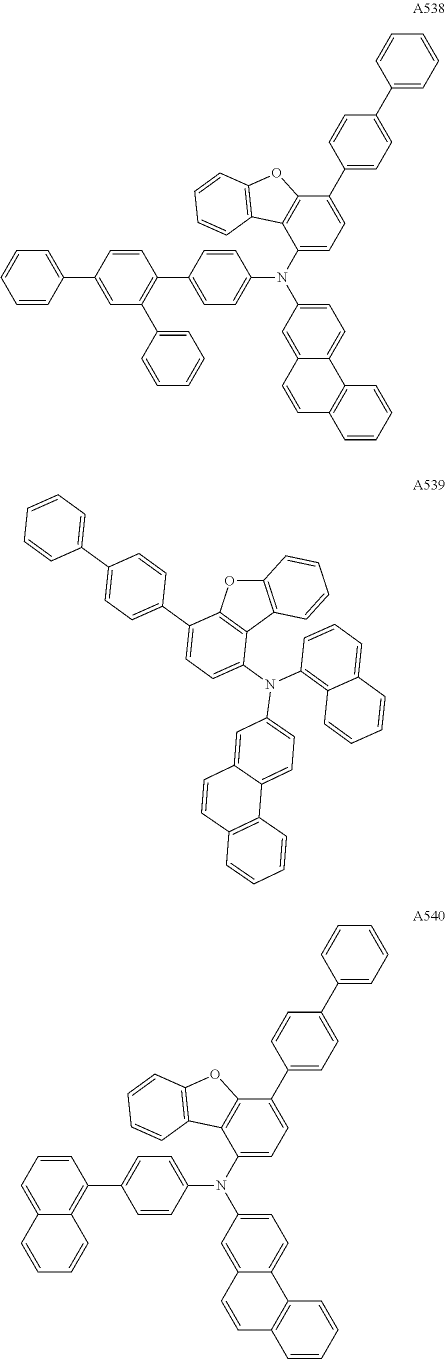 [SMILES: c1ccc(-c2ccc(-c3ccc(N(c4ccc(-c5ccc(-c6ccccc6)cc5-c5ccccc5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4cccc5ccccc45)c4c3oc3ccccc34)cc2)cc1 ; c1ccc(-c2ccc(-c3ccc(N(c4ccc(-c5cccc6ccccc56)cc4)c4ccc5c(ccc6ccccc65)c4)c4c3oc3ccccc34)cc2)cc1] 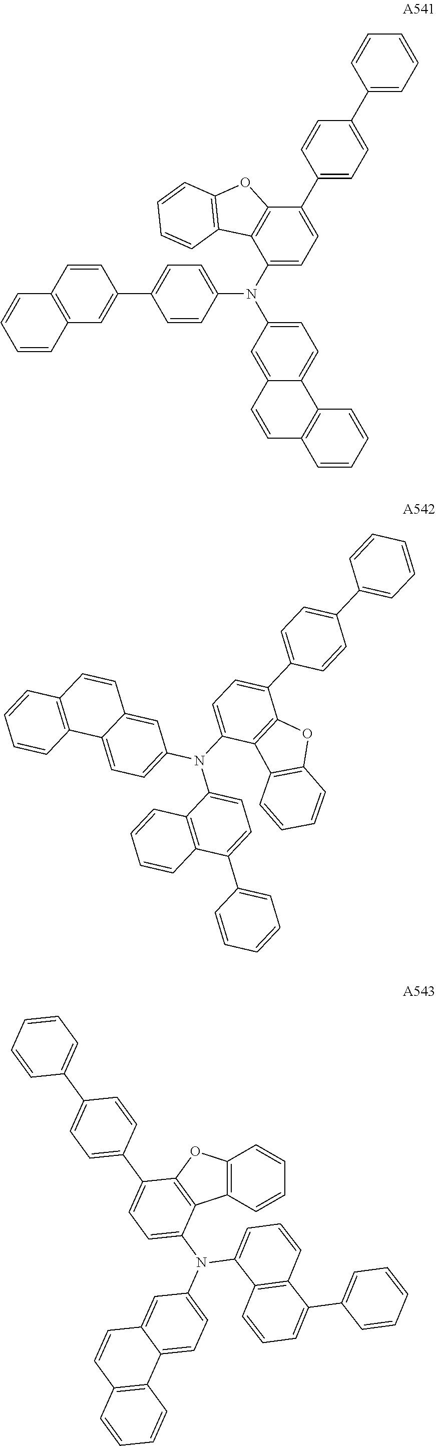 [SMILES: c1ccc(-c2ccc(-c3ccc(N(c4ccc(-c5ccc6ccccc6c5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccccc5)c5ccccc45)c4c3oc3ccccc34)cc2)cc1 ; c1ccc(-c2ccc(-c3ccc(N(c4ccc5c(ccc6ccccc65)c4)c4cccc5c(-c6ccccc6)cccc45)c4c3oc3ccccc34)cc2)cc1] 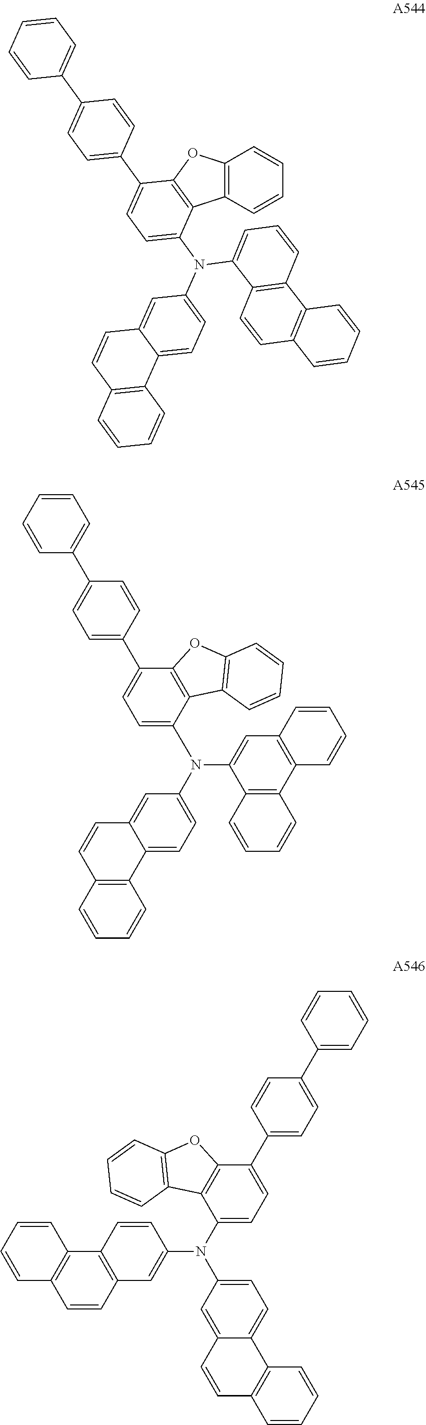 [SMILES: c1ccc(-c2ccc(-c3ccc(N(c4ccc5c(ccc6ccccc65)c4)c4cccc5c4ccc4ccccc45)c4c3oc3ccccc34)cc2)cc1 ; c1ccc(-c2ccc(-c3ccc(N(c4ccc5c(ccc6ccccc65)c4)c4cc5ccccc5c5ccccc45)c4c3oc3ccccc34)cc2)cc1 ; c1ccc(-c2ccc(-c3ccc(N(c4ccc5c(ccc6ccccc65)c4)c4ccc5c(ccc6ccccc65)c4)c4c3oc3ccccc34)cc2)cc1] 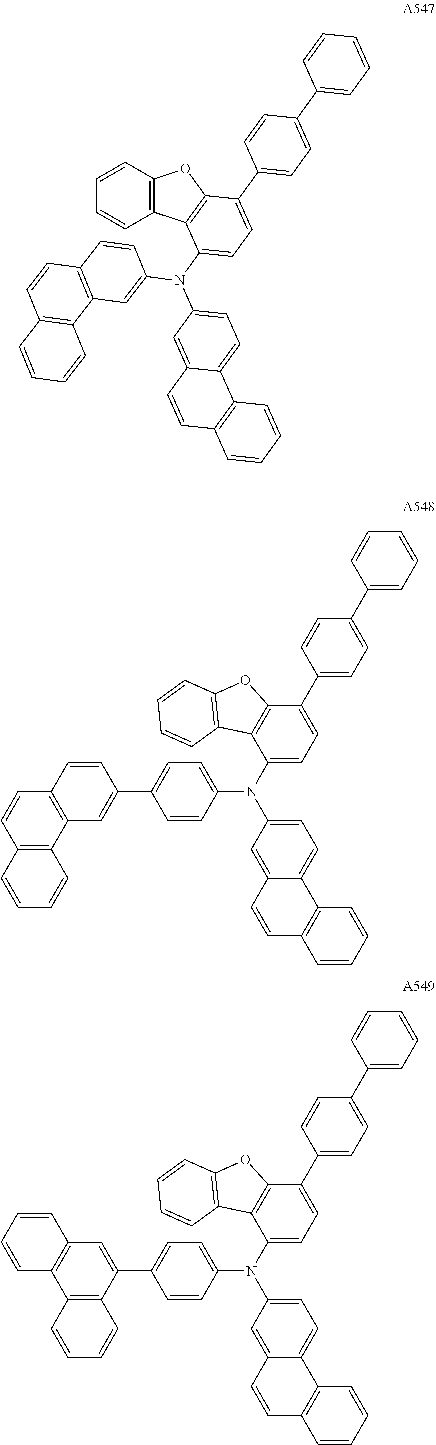 [SMILES: c1ccc(-c2ccc(-c3ccc(N(c4ccc5c(ccc6ccccc65)c4)c4ccc5ccc6ccccc6c5c4)c4c3oc3ccccc34)cc2)cc1 ; c1ccc(-c2ccc(-c3ccc(N(c4ccc(-c5ccc6ccc7ccccc7c6c5)cc4)c4ccc5c(ccc6ccccc65)c4)c4c3oc3ccccc34)cc2)cc1 ; c1ccc(-c2ccc(-c3ccc(N(c4ccc(-c5cc6ccccc6c6ccccc56)cc4)c4ccc5c(ccc6ccccc65)c4)c4c3oc3ccccc34)cc2)cc1] 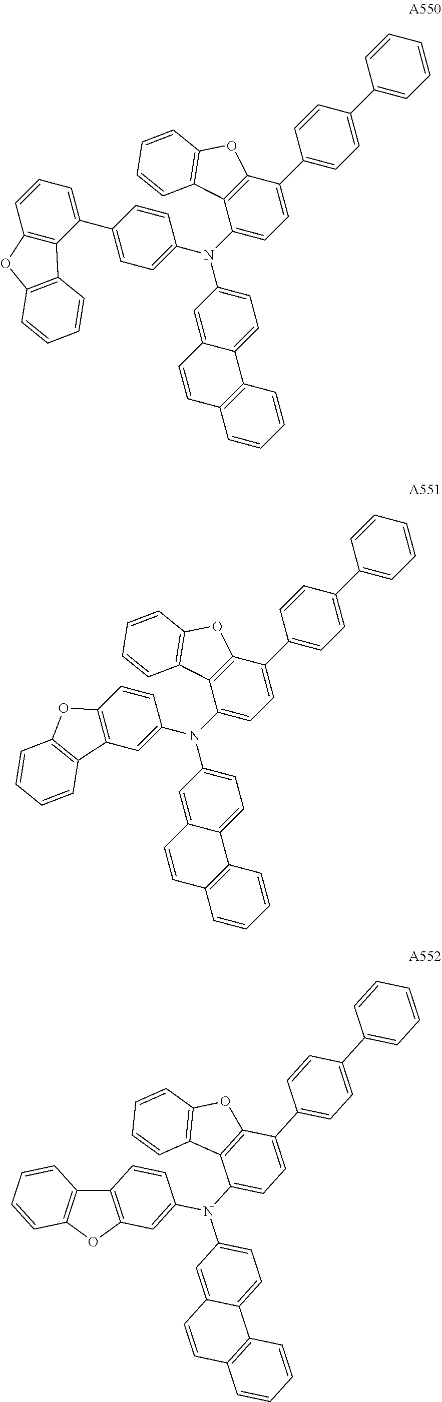 [SMILES: c1ccc(-c2ccc(-c3ccc(N(c4ccc(-c5cccc6oc7ccccc7c56)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc5oc6ccccc6c5c4)c4c3oc3ccccc34)cc2)cc1 ; c1ccc(-c2ccc(-c3ccc(N(c4ccc5c(ccc6ccccc65)c4)c4ccc5c(c4)oc4ccccc45)c4c3oc3ccccc34)cc2)cc1] 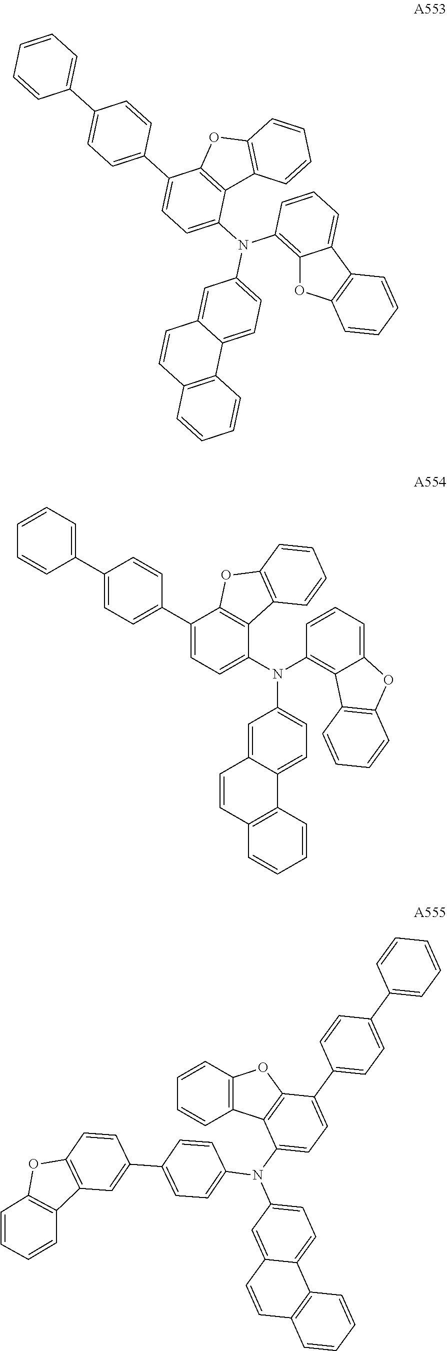 [SMILES: c1ccc(-c2ccc(-c3ccc(N(c4ccc5c(ccc6ccccc65)c4)c4cccc5c4oc4ccccc45)c4c3oc3ccccc34)cc2)cc1 ; c1ccc(-c2ccc(-c3ccc(N(c4ccc5c(ccc6ccccc65)c4)c4cccc5oc6ccccc6c45)c4c3oc3ccccc34)cc2)cc1 ; c1ccc(-c2ccc(-c3ccc(N(c4ccc(-c5ccc6oc7ccccc7c6c5)cc4)c4ccc5c(ccc6ccccc65)c4)c4c3oc3ccccc34)cc2)cc1] 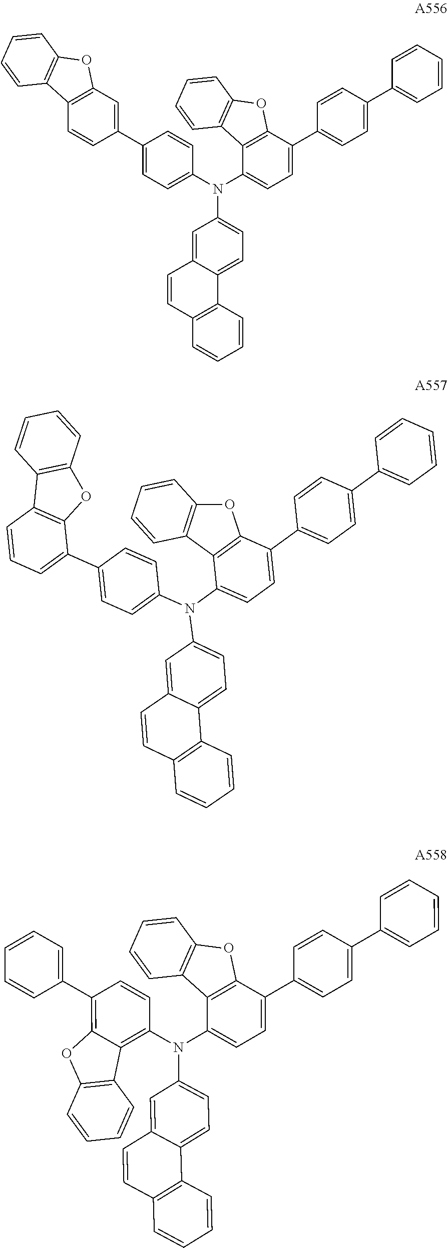 [SMILES: c1ccc(-c2ccc(-c3ccc(N(c4ccc(-c5ccc6c(c5)oc5ccccc56)cc4)c4ccc5c(ccc6ccccc65)c4)c4c3oc3ccccc34)cc2)cc1 ; c1ccc(-c2ccc(-c3ccc(N(c4ccc(-c5cccc6c5oc5ccccc56)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccccc5)c5oc6ccccc6c45)c4c3oc3ccccc34)cc2)cc1] 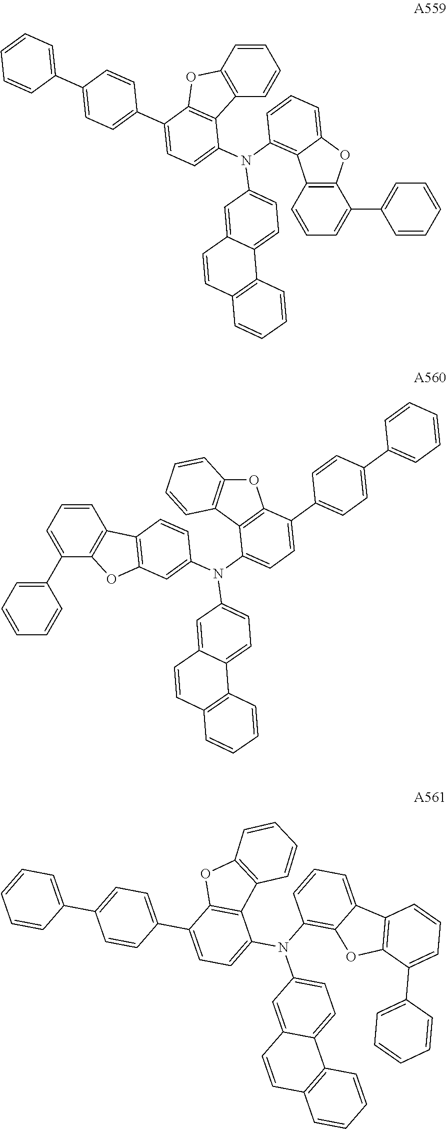 [SMILES: c1ccc(-c2ccc(-c3ccc(N(c4ccc5c(ccc6ccccc65)c4)c4cccc5oc6c(-c7ccccc7)cccc6c45)c4c3oc3ccccc34)cc2)cc1 ; c1ccc(-c2ccc(-c3ccc(N(c4ccc5c(ccc6ccccc65)c4)c4ccc5c(c4)oc4c(-c6ccccc6)cccc45)c4c3oc3ccccc34)cc2)cc1 ; c1ccc(-c2ccc(-c3ccc(N(c4ccc5c(ccc6ccccc65)c4)c4cccc5c4oc4c(-c6ccccc6)cccc45)c4c3oc3ccccc34)cc2)cc1] 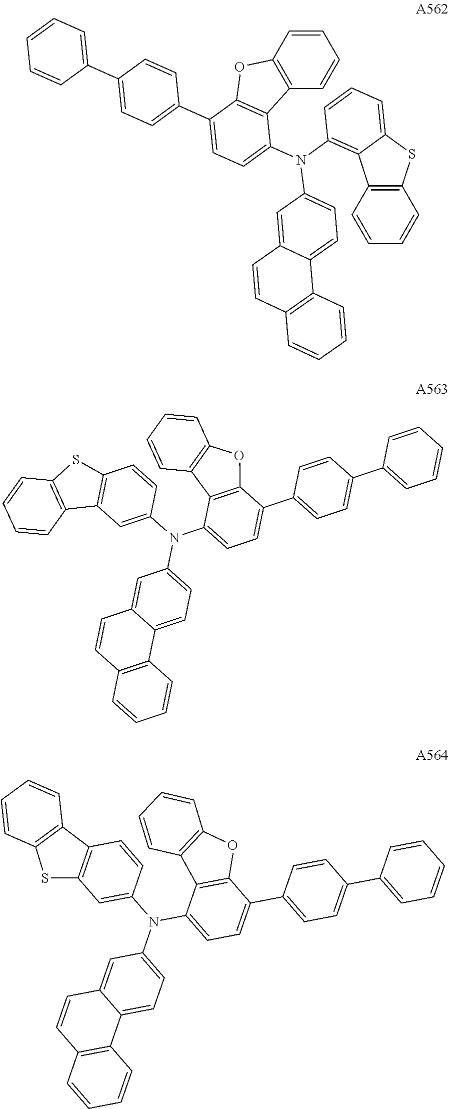 [SMILES: c1ccc(-c2ccc(-c3ccc(N(c4ccc5c(ccc6ccccc65)c4)c4cccc5sc6ccccc6c45)c4c3oc3ccccc34)cc2)cc1 ; c1ccc(-c2ccc(-c3ccc(N(c4ccc5c(ccc6ccccc65)c4)c4ccc5sc6ccccc6c5c4)c4c3oc3ccccc34)cc2)cc1 ; c1ccc(-c2ccc(-c3ccc(N(c4ccc5c(ccc6ccccc65)c4)c4ccc5c(c4)sc4ccccc45)c4c3oc3ccccc34)cc2)cc1] 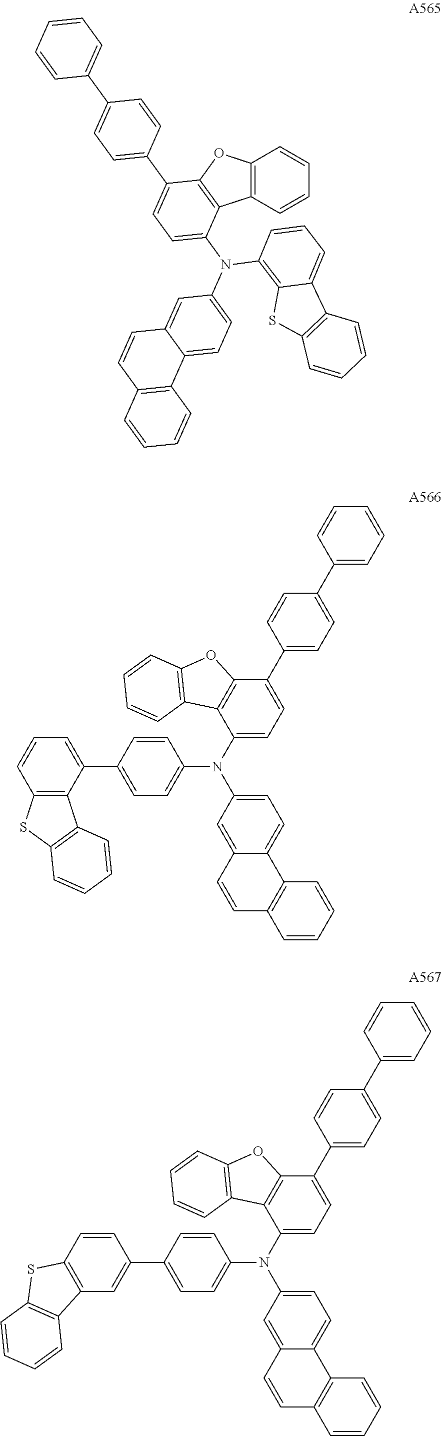 [SMILES: c1ccc(-c2ccc(-c3ccc(N(c4ccc5c(ccc6ccccc65)c4)c4cccc5c4sc4ccccc45)c4c3oc3ccccc34)cc2)cc1 ; c1ccc(-c2ccc(-c3ccc(N(c4ccc(-c5cccc6sc7ccccc7c56)cc4)c4ccc5c(ccc6ccccc65)c4)c4c3oc3ccccc34)cc2)cc1 ; c1ccc(-c2ccc(-c3ccc(N(c4ccc(-c5ccc6sc7ccccc7c6c5)cc4)c4ccc5c(ccc6ccccc65)c4)c4c3oc3ccccc34)cc2)cc1] 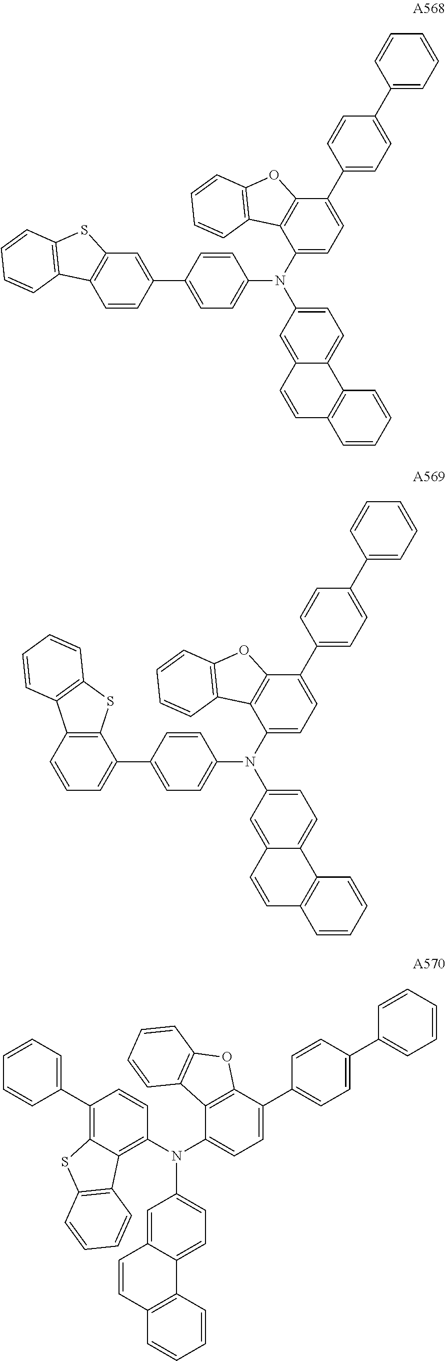 [SMILES: c1ccc(-c2ccc(-c3ccc(N(c4ccc(-c5ccc6c(c5)sc5ccccc56)cc4)c4ccc5c(ccc6ccccc65)c4)c4c3oc3ccccc34)cc2)cc1 ; c1ccc(-c2ccc(-c3ccc(N(c4ccc(-c5cccc6c5sc5ccccc56)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccccc5)c5sc6ccccc6c45)c4c3oc3ccccc34)cc2)cc1] 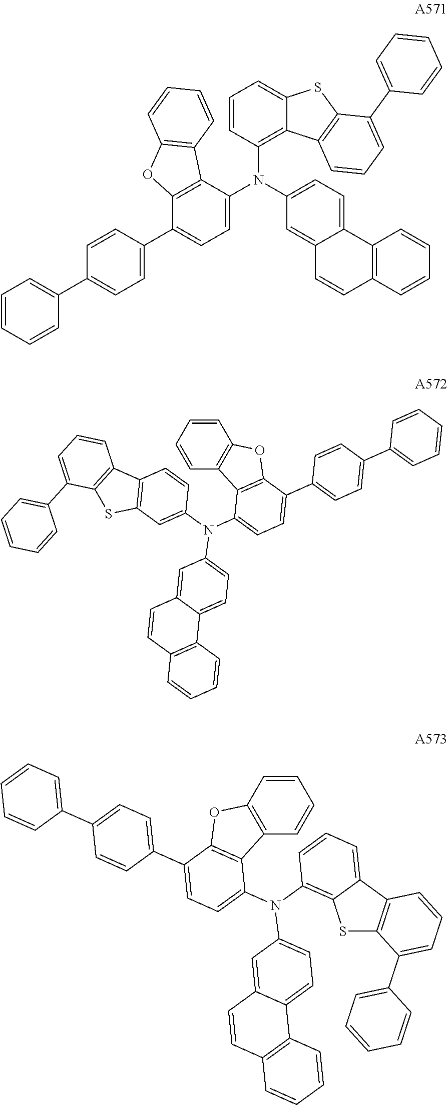 [SMILES: c1ccc(-c2ccc(-c3ccc(N(c4ccc5c(ccc6ccccc65)c4)c4cccc5sc6c(-c7ccccc7)cccc6c45)c4c3oc3ccccc34)cc2)cc1 ; c1ccc(-c2ccc(-c3ccc(N(c4ccc5c(ccc6ccccc65)c4)c4ccc5c(c4)sc4c(-c6ccccc6)cccc45)c4c3oc3ccccc34)cc2)cc1 ; c1ccc(-c2ccc(-c3ccc(N(c4ccc5c(ccc6ccccc65)c4)c4cccc5c4sc4c(-c6ccccc6)cccc45)c4c3oc3ccccc34)cc2)cc1] 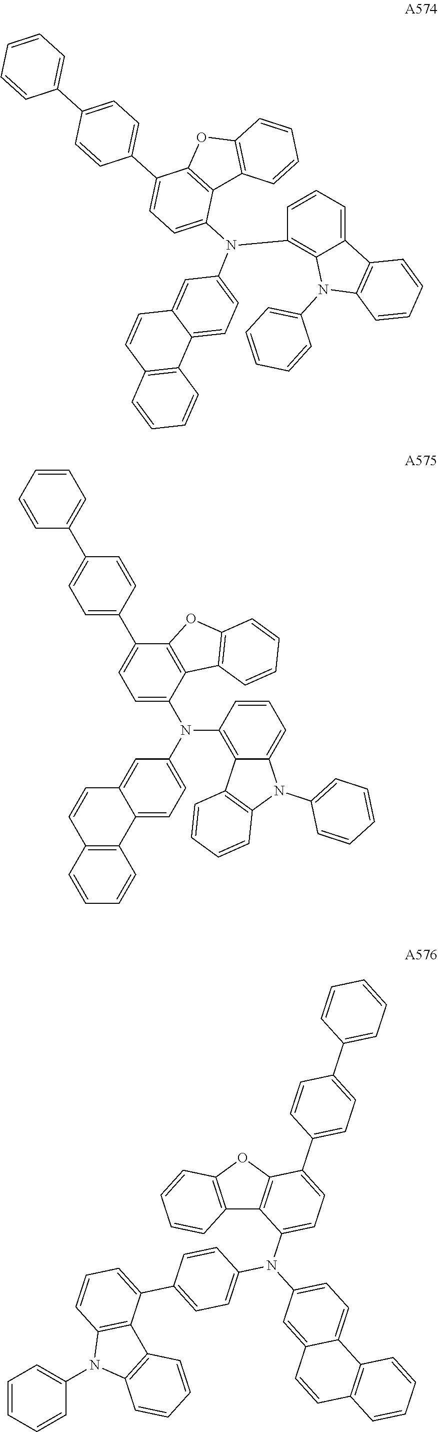 [SMILES: c1ccc(-c2ccc(-c3ccc(N(c4ccc5c(ccc6ccccc65)c4)c4cccc5c6ccccc6n(-c6ccccc6)c45)c4c3oc3ccccc34)cc2)cc1 ; c1ccc(-c2ccc(-c3ccc(N(c4ccc5c(ccc6ccccc65)c4)c4cccc5c4c4ccccc4n5-c4ccccc4)c4c3oc3ccccc34)cc2)cc1 ; c1ccc(-c2ccc(-c3ccc(N(c4ccc(-c5cccc6c5c5ccccc5n6-c5ccccc5)cc4)c4ccc5c(ccc6ccccc65)c4)c4c3oc3ccccc34)cc2)cc1] 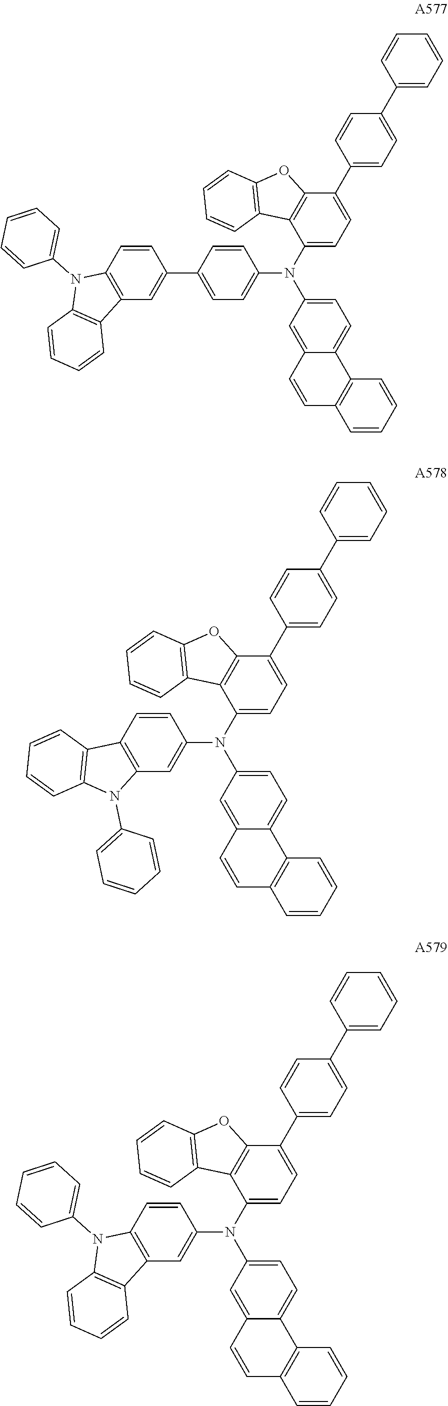 [SMILES: c1ccc(-c2ccc(-c3ccc(N(c4ccc(-c5ccc6c(c5)c5ccccc5n6-c5ccccc5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc5c6ccccc6n(-c6ccccc6)c5c4)c4c3oc3ccccc34)cc2)cc1 ; c1ccc(-c2ccc(-c3ccc(N(c4ccc5c(ccc6ccccc65)c4)c4ccc5c(c4)c4ccccc4n5-c4ccccc4)c4c3oc3ccccc34)cc2)cc1] 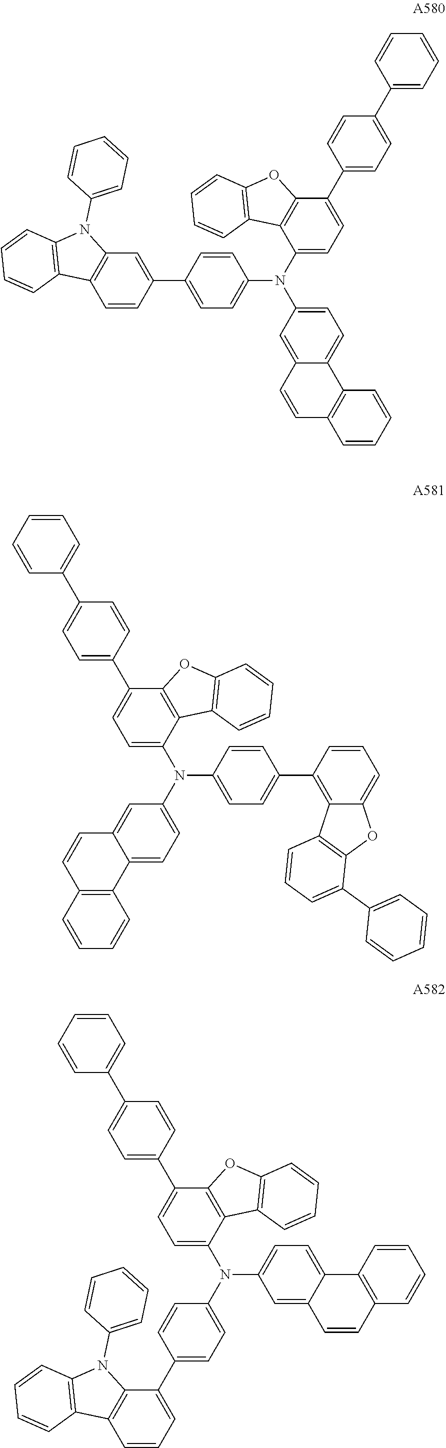 [SMILES: c1ccc(-c2ccc(-c3ccc(N(c4ccc(-c5ccc6c7ccccc7n(-c7ccccc7)c6c5)cc4)c4ccc5c(ccc6ccccc65)c4)c4c3oc3ccccc34)cc2)cc1 ; c1ccc(-c2ccc(-c3ccc(N(c4ccc(-c5cccc6oc7c(-c8ccccc8)cccc7c56)cc4)c4ccc5c(ccc6ccccc65)c4)c4c3oc3ccccc34)cc2)cc1 ; c1ccc(-c2ccc(-c3ccc(N(c4ccc(-c5cccc6c7ccccc7n(-c7ccccc7)c56)cc4)c4ccc5c(ccc6ccccc65)c4)c4c3oc3ccccc34)cc2)cc1] 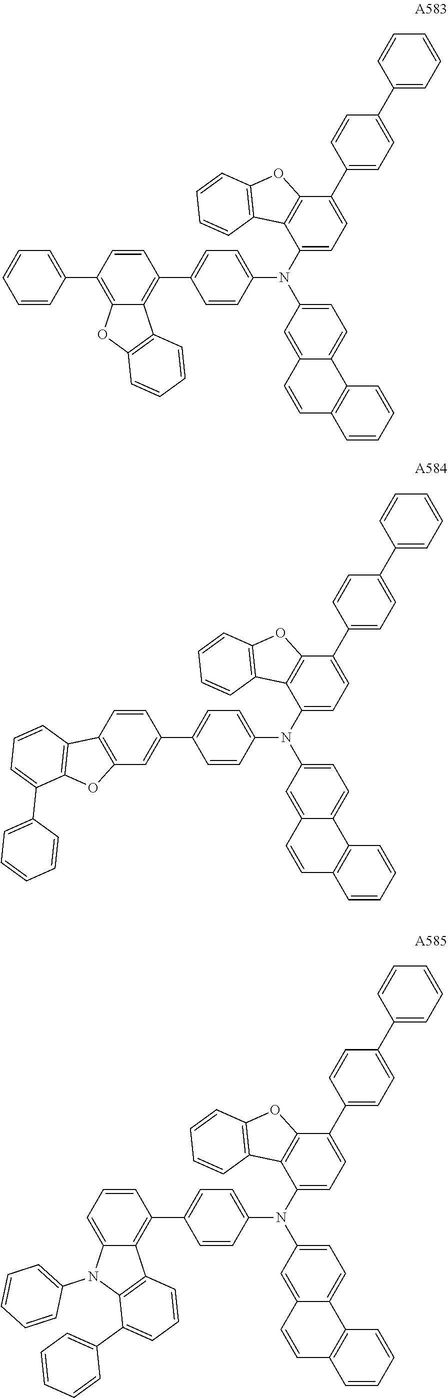 [SMILES: c1ccc(-c2ccc(-c3ccc(N(c4ccc(-c5ccc(-c6ccccc6)c6oc7ccccc7c56)cc4)c4ccc5c(ccc6ccccc65)c4)c4c3oc3ccccc34)cc2)cc1 ; c1ccc(-c2ccc(-c3ccc(N(c4ccc(-c5ccc6c(c5)oc5c(-c7ccccc7)cccc56)cc4)c4ccc5c(ccc6ccccc65)c4)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4ccc5c(ccc6ccccc65)c4)c4c3oc3ccccc34)cc2)cc1] 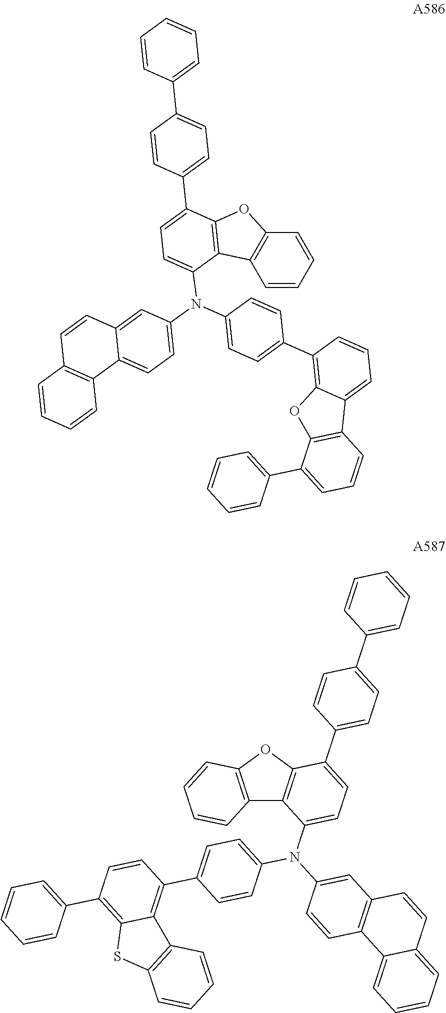 [SMILES: c1ccc(-c2ccc(-c3ccc(N(c4ccc(-c5cccc6c5oc5c(-c7ccccc7)cccc56)cc4)c4ccc5c(ccc6ccccc65)c4)c4c3oc3ccccc34)cc2)cc1 ; c1ccc(-c2ccc(-c3ccc(N(c4ccc(-c5ccc(-c6ccccc6)c6sc7ccccc7c56)cc4)c4ccc5c(ccc6ccccc65)c4)c4c3oc3ccccc34)cc2)cc1] 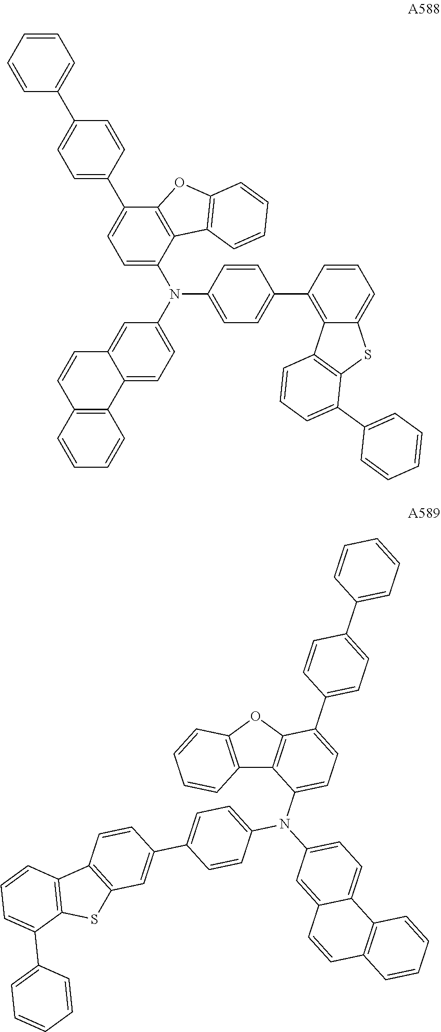 [SMILES: c1ccc(-c2ccc(-c3ccc(N(c4ccc(-c5cccc6sc7c(-c8ccccc8)cccc7c56)cc4)c4ccc5c(ccc6ccccc65)c4)c4c3oc3ccccc34)cc2)cc1 ; c1ccc(-c2ccc(-c3ccc(N(c4ccc(-c5ccc6c(c5)sc5c(-c7ccccc7)cccc56)cc4)c4ccc5c(ccc6ccccc65)c4)c4c3oc3ccccc34)cc2)cc1] 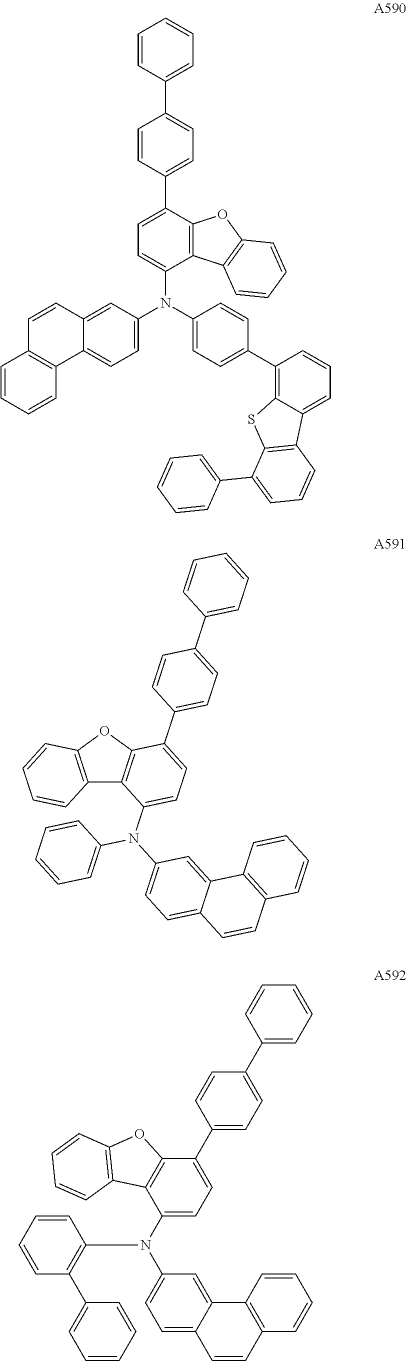 [SMILES: c1ccc(-c2ccc(-c3ccc(N(c4ccc(-c5cccc6c5sc5c(-c7ccccc7)cccc56)cc4)c4ccc5c(ccc6ccccc65)c4)c4c3oc3ccccc34)cc2)cc1 ; c1ccc(-c2ccc(-c3ccc(N(c4ccccc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccccc4-c4ccccc4)c4c3oc3ccccc34)cc2)cc1] 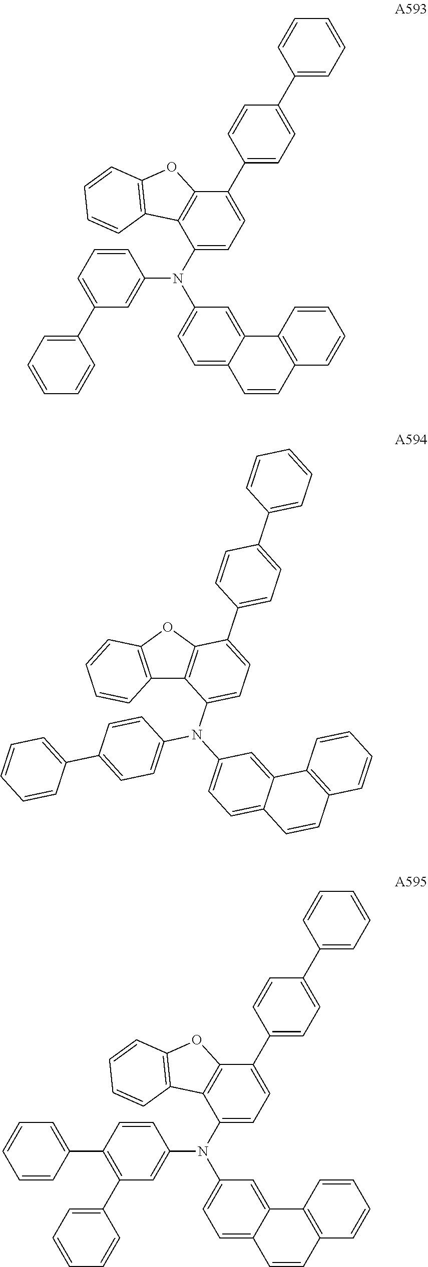 [SMILES: c1ccc(-c2ccc(-c3ccc(N(c4cccc(-c5ccccc5)c4)c4ccc5ccc6ccccc6c5c4)c4c3oc3ccccc34)cc2)cc1 ; c1ccc(-c2ccc(-c3ccc(N(c4ccc(-c5ccccc5)cc4)c4ccc5ccc6ccccc6c5c4)c4c3oc3ccccc34)cc2)cc1 ; c1ccc(-c2ccc(-c3ccc(N(c4ccc(-c5ccccc5)c(-c5ccccc5)c4)c4ccc5ccc6ccccc6c5c4)c4c3oc3ccccc34)cc2)cc1] 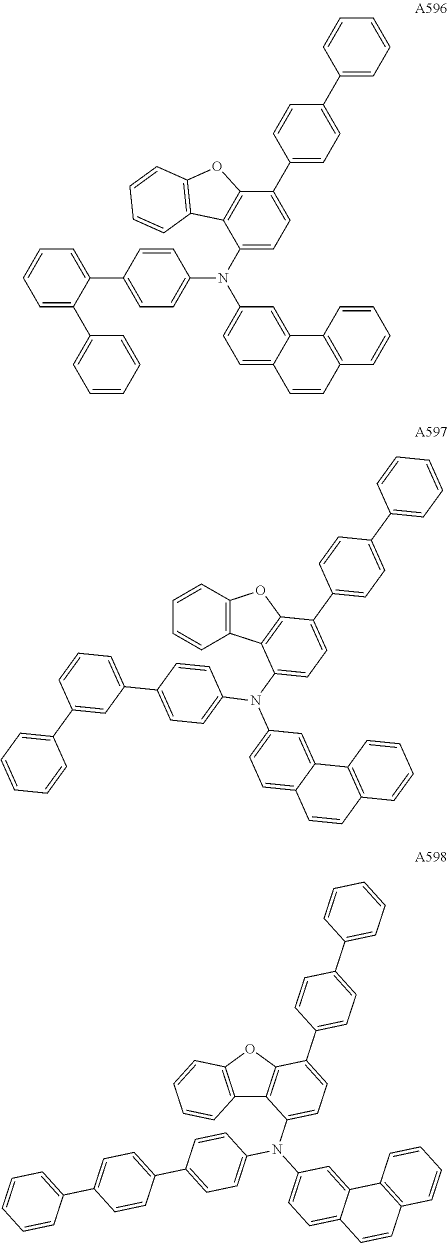 [SMILES: c1ccc(-c2ccc(-c3ccc(N(c4ccc(-c5ccccc5-c5ccccc5)cc4)c4ccc5ccc6ccccc6c5c4)c4c3oc3ccccc34)cc2)cc1 ; c1ccc(-c2ccc(-c3ccc(N(c4ccc(-c5cccc(-c6ccccc6)c5)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccc(-c6ccccc6)cc5)c5oc6ccccc6c45)cc3)cc2)cc1] 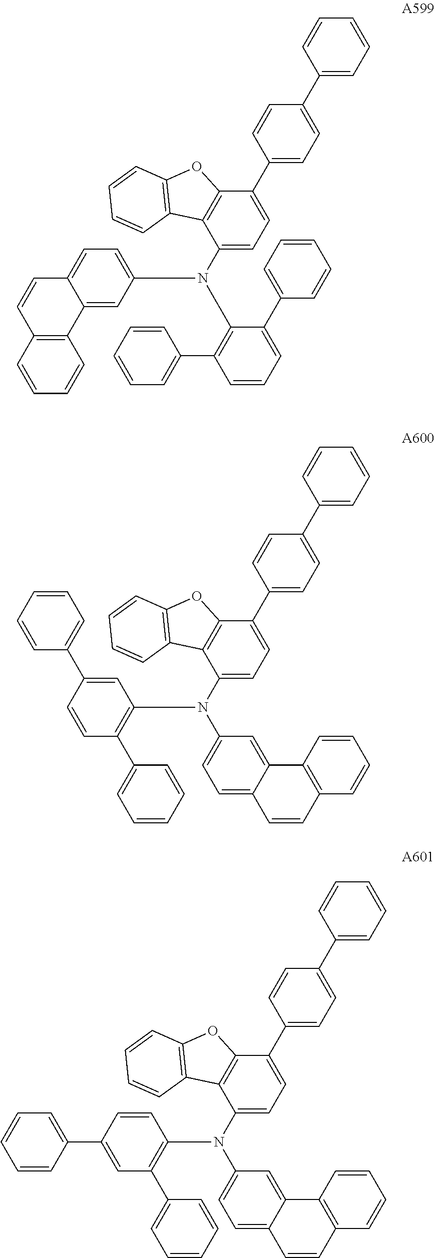 [SMILES: c1ccc(-c2ccc(-c3ccc(N(c4ccc5ccc6ccccc6c5c4)c4c(-c5ccccc5)cccc4-c4ccccc4)c4c3oc3ccccc34)cc2)cc1 ; c1ccc(-c2ccc(-c3ccc(N(c4ccc5ccc6ccccc6c5c4)c4cc(-c5ccccc5)ccc4-c4ccccc4)c4c3oc3ccccc34)cc2)cc1 ; c1ccc(-c2ccc(-c3ccc(N(c4ccc5ccc6ccccc6c5c4)c4ccc(-c5ccccc5)cc4-c4ccccc4)c4c3oc3ccccc34)cc2)cc1] 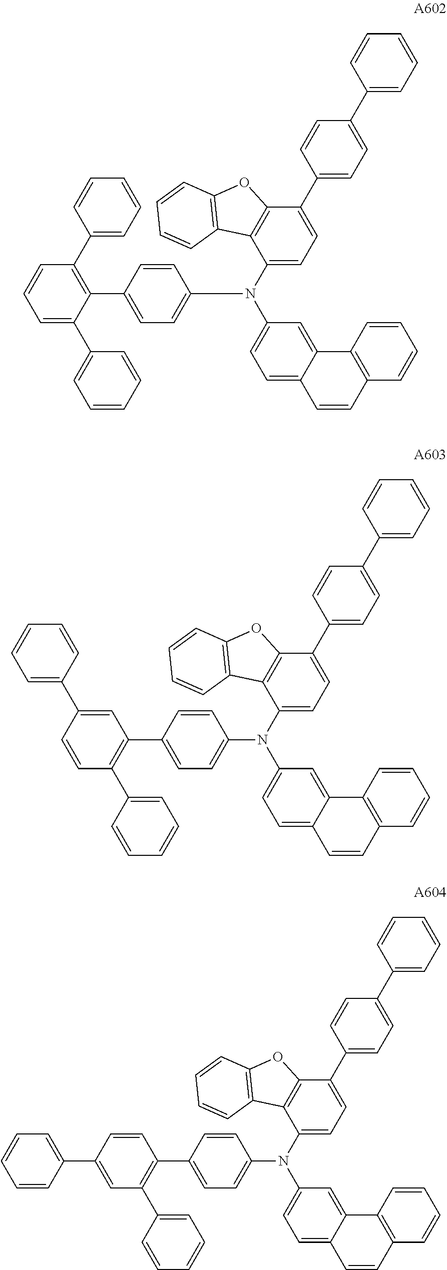 [SMILES: c1ccc(-c2ccc(-c3ccc(N(c4ccc(-c5c(-c6ccccc6)cccc5-c5ccccc5)cc4)c4ccc5ccc6ccccc6c5c4)c4c3oc3ccccc34)cc2)cc1 ; c1ccc(-c2ccc(-c3ccc(N(c4ccc(-c5cc(-c6ccccc6)ccc5-c5ccccc5)cc4)c4ccc5ccc6ccccc6c5c4)c4c3oc3ccccc34)cc2)cc1 ; c1ccc(-c2ccc(-c3ccc(N(c4ccc(-c5ccc(-c6ccccc6)cc5-c5ccccc5)cc4)c4ccc5ccc6ccccc6c5c4)c4c3oc3ccccc34)cc2)cc1] 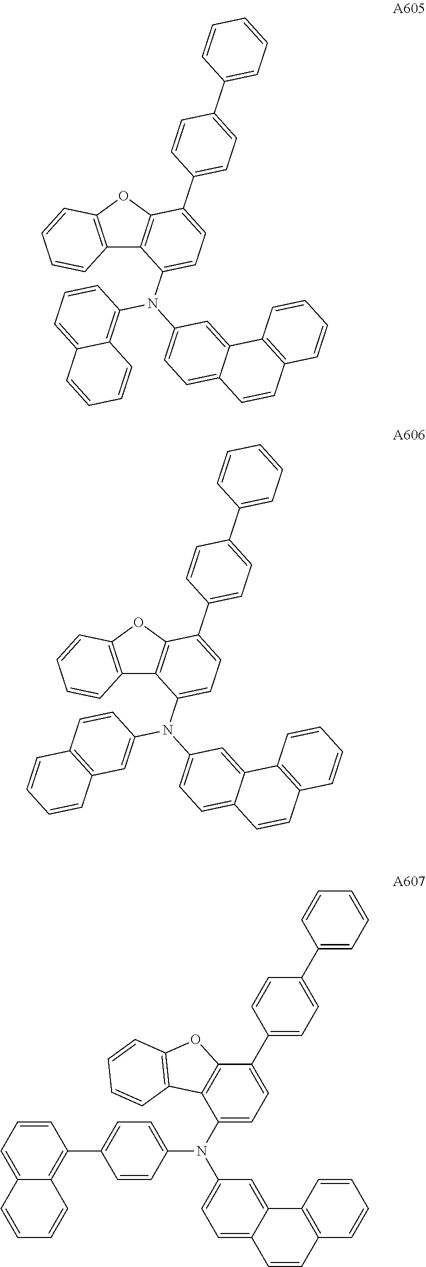 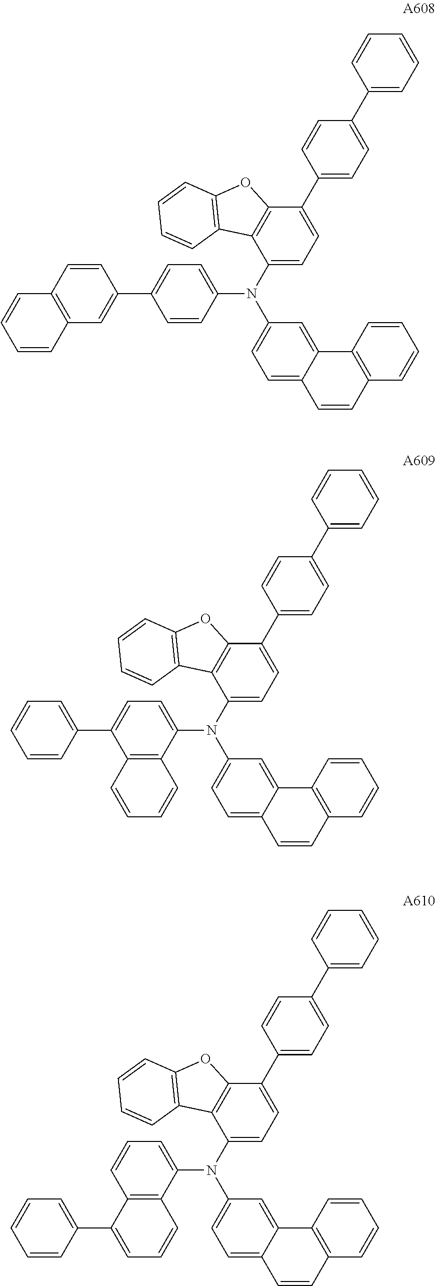 [SMILES: c1ccc(-c2ccc(-c3ccc(N(c4ccc(-c5ccc6ccccc6c5)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccccc5)c5ccccc45)c4c3oc3ccccc34)cc2)cc1 ; c1ccc(-c2ccc(-c3ccc(N(c4ccc5ccc6ccccc6c5c4)c4cccc5c(-c6ccccc6)cccc45)c4c3oc3ccccc34)cc2)cc1] 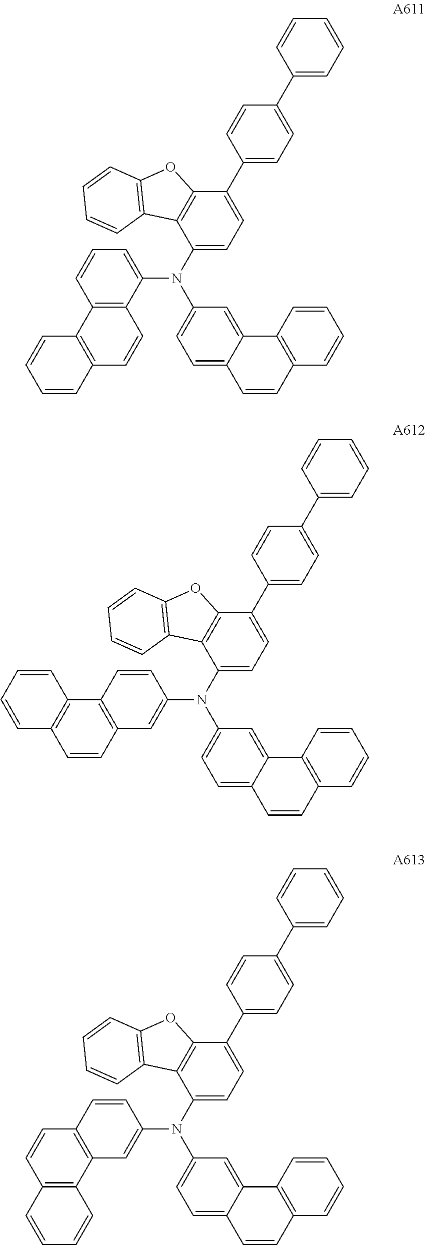 [SMILES: c1ccc(-c2ccc(-c3ccc(N(c4ccc5ccc6ccccc6c5c4)c4cccc5c4ccc4ccccc45)c4c3oc3ccccc34)cc2)cc1 ; c1ccc(-c2ccc(-c3ccc(N(c4ccc5c(ccc6ccccc65)c4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc5ccc6ccccc6c5c4)c4c3oc3ccccc34)cc2)cc1] 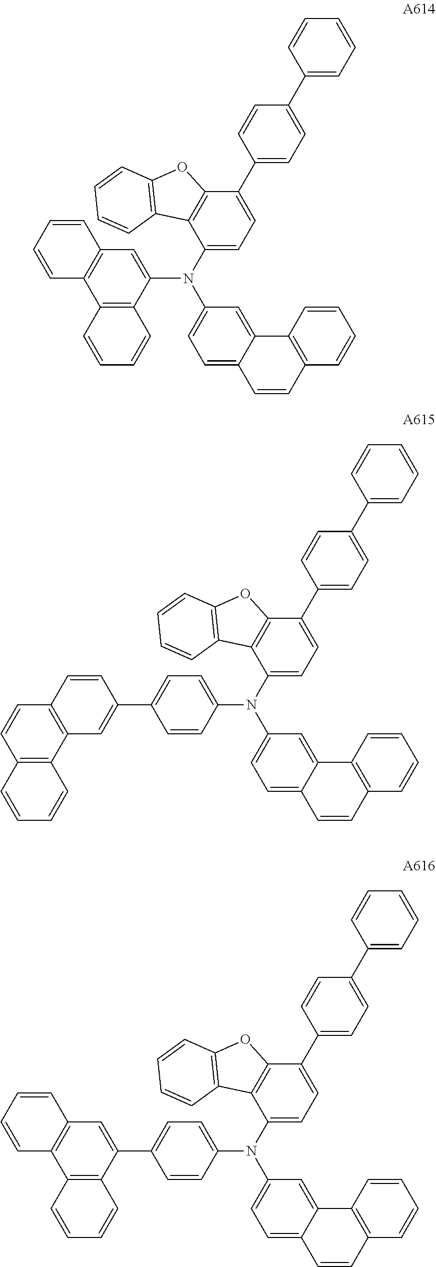 [SMILES: c1ccc(-c2ccc(-c3ccc(N(c4ccc5ccc6ccccc6c5c4)c4cc5ccccc5c5ccccc45)c4c3oc3ccccc34)cc2)cc1 ; c1ccc(-c2ccc(-c3ccc(N(c4ccc(-c5ccc6ccc7ccccc7c6c5)cc4)c4ccc5ccc6ccccc6c5c4)c4c3oc3ccccc34)cc2)cc1 ; c1ccc(-c2ccc(-c3ccc(N(c4ccc(-c5cc6ccccc6c6ccccc56)cc4)c4ccc5ccc6ccccc6c5c4)c4c3oc3ccccc34)cc2)cc1] 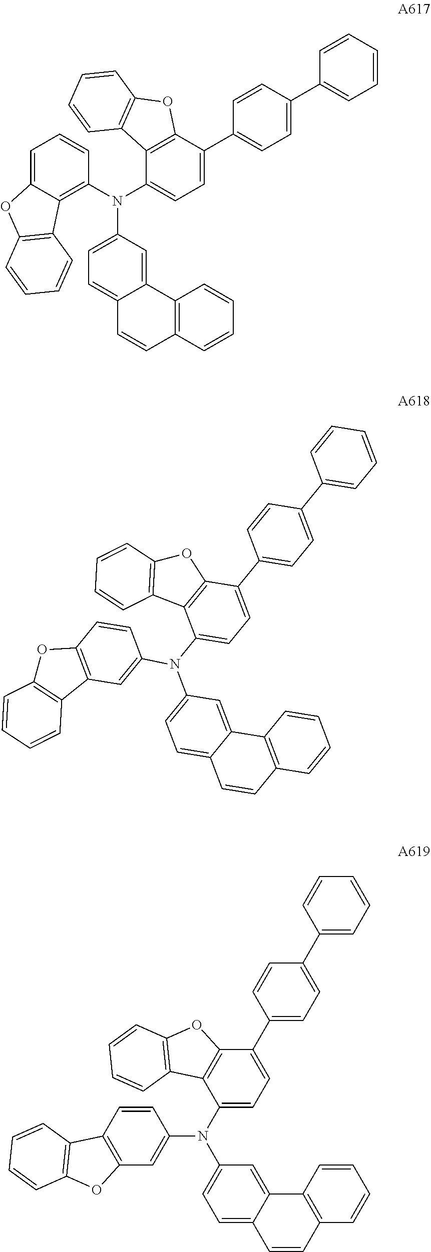 [SMILES: c1ccc(-c2ccc(-c3ccc(N(c4ccc5ccc6ccccc6c5c4)c4cccc5oc6ccccc6c45)c4c3oc3ccccc34)cc2)cc1 ; c1ccc(-c2ccc(-c3ccc(N(c4ccc5ccc6ccccc6c5c4)c4ccc5oc6ccccc6c5c4)c4c3oc3ccccc34)cc2)cc1 ; c1ccc(-c2ccc(-c3ccc(N(c4ccc5c(c4)oc4ccccc45)c4ccc5ccc6ccccc6c5c4)c4c3oc3ccccc34)cc2)cc1] 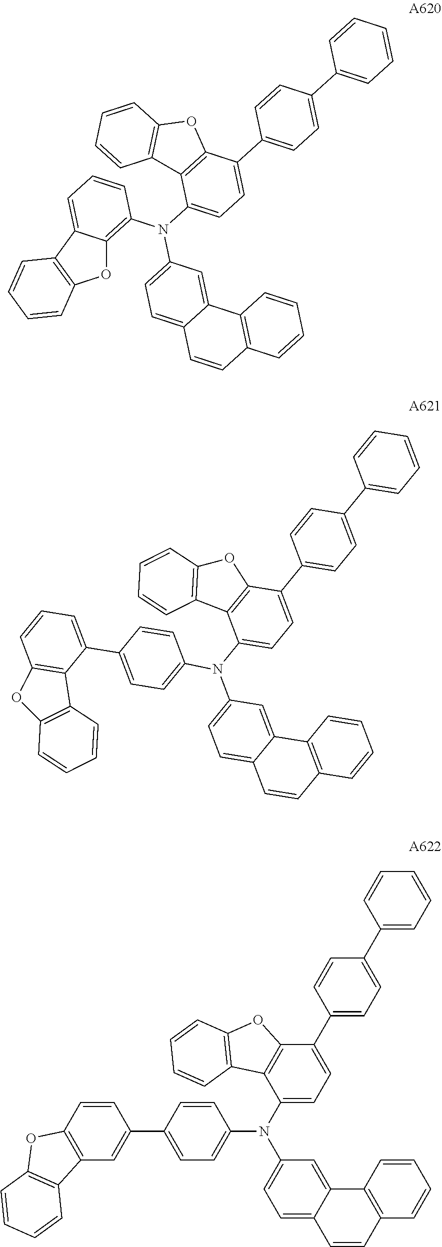 [SMILES: c1ccc(-c2ccc(-c3ccc(N(c4ccc5ccc6ccccc6c5c4)c4cccc5c4oc4ccccc45)c4c3oc3ccccc34)cc2)cc1 ; c1ccc(-c2ccc(-c3ccc(N(c4ccc(-c5cccc6oc7ccccc7c56)cc4)c4ccc5ccc6ccccc6c5c4)c4c3oc3ccccc34)cc2)cc1 ; c1ccc(-c2ccc(-c3ccc(N(c4ccc(-c5ccc6oc7ccccc7c6c5)cc4)c4ccc5ccc6ccccc6c5c4)c4c3oc3ccccc34)cc2)cc1] 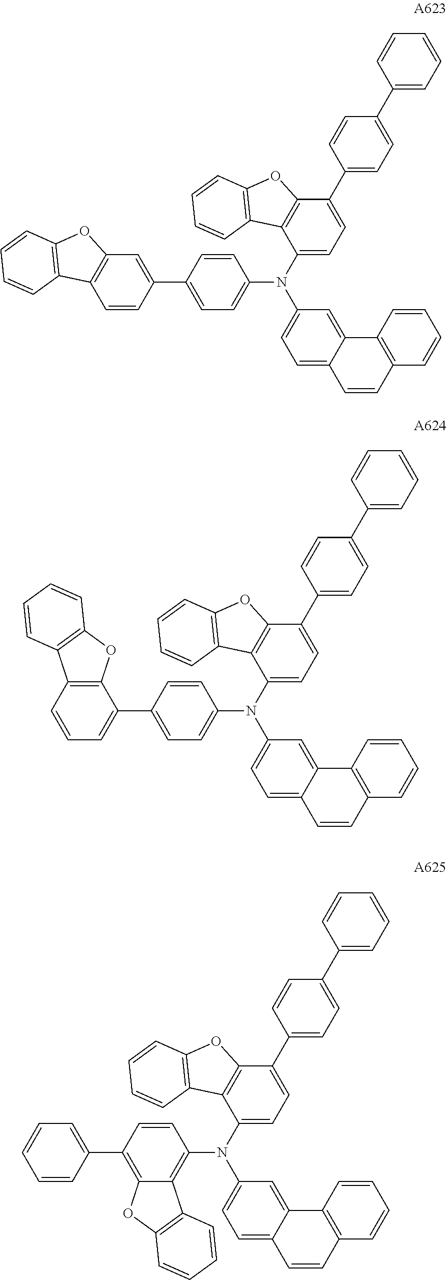 [SMILES: c1ccc(-c2ccc(-c3ccc(N(c4ccc(-c5ccc6c(c5)oc5ccccc56)cc4)c4ccc5ccc6ccccc6c5c4)c4c3oc3ccccc34)cc2)cc1 ; c1ccc(-c2ccc(-c3ccc(N(c4ccc(-c5cccc6c5oc5ccccc56)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccccc5)c5oc6ccccc6c45)c4c3oc3ccccc34)cc2)cc1] 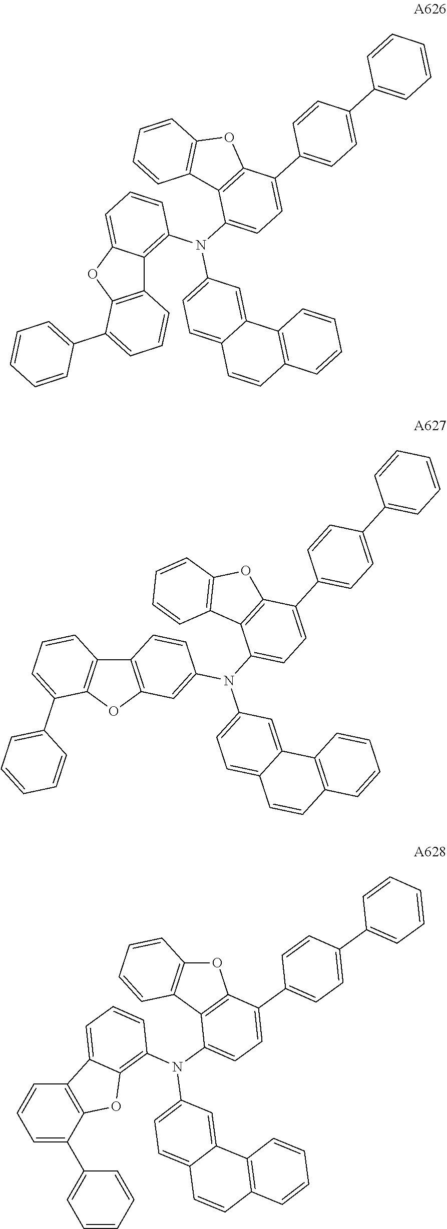 [SMILES: c1ccc(-c2ccc(-c3ccc(N(c4ccc5ccc6ccccc6c5c4)c4cccc5oc6c(-c7ccccc7)cccc6c45)c4c3oc3ccccc34)cc2)cc1 ; c1ccc(-c2ccc(-c3ccc(N(c4ccc5c(c4)oc4c(-c6ccccc6)cccc45)c4ccc5ccc6ccccc6c5c4)c4c3oc3ccccc34)cc2)cc1 ; c1ccc(-c2ccc(-c3ccc(N(c4ccc5ccc6ccccc6c5c4)c4cccc5c4oc4c(-c6ccccc6)cccc45)c4c3oc3ccccc34)cc2)cc1] 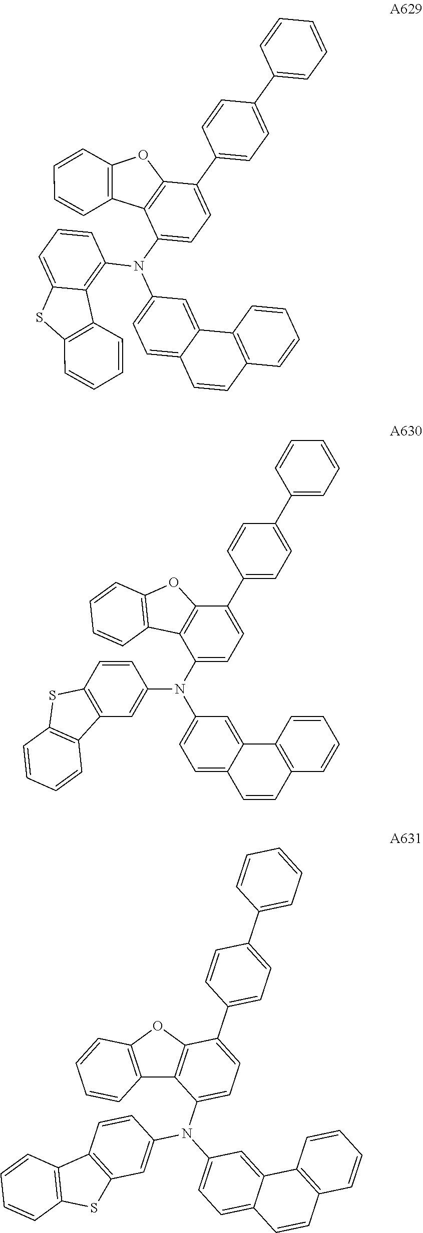 [SMILES: c1ccc(-c2ccc(-c3ccc(N(c4ccc5ccc6ccccc6c5c4)c4cccc5sc6ccccc6c45)c4c3oc3ccccc34)cc2)cc1 ; c1ccc(-c2ccc(-c3ccc(N(c4ccc5ccc6ccccc6c5c4)c4ccc5sc6ccccc6c5c4)c4c3oc3ccccc34)cc2)cc1 ; c1ccc(-c2ccc(-c3ccc(N(c4ccc5c(c4)sc4ccccc45)c4ccc5ccc6ccccc6c5c4)c4c3oc3ccccc34)cc2)cc1] 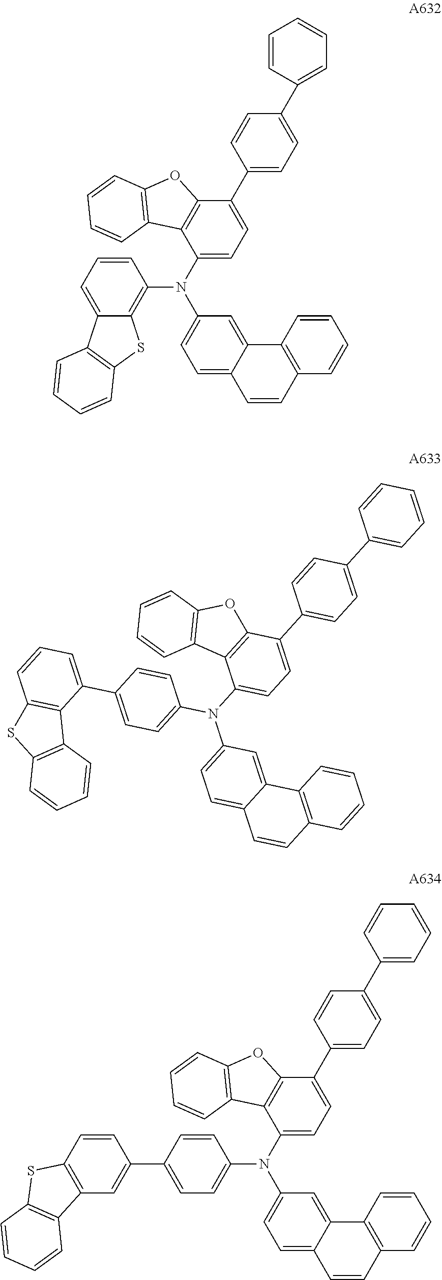 [SMILES: c1ccc(-c2ccc(-c3ccc(N(c4ccc5ccc6ccccc6c5c4)c4cccc5c4sc4ccccc45)c4c3oc3ccccc34)cc2)cc1 ; c1ccc(-c2ccc(-c3ccc(N(c4ccc(-c5cccc6sc7ccccc7c56)cc4)c4ccc5ccc6ccccc6c5c4)c4c3oc3ccccc34)cc2)cc1 ; c1ccc(-c2ccc(-c3ccc(N(c4ccc(-c5ccc6sc7ccccc7c6c5)cc4)c4ccc5ccc6ccccc6c5c4)c4c3oc3ccccc34)cc2)cc1] 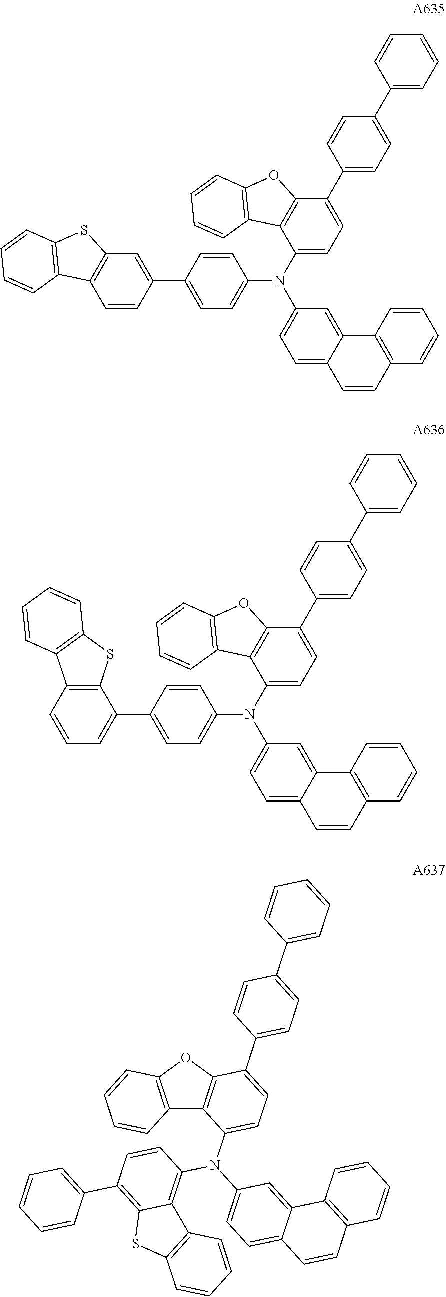 [SMILES: c1ccc(-c2ccc(-c3ccc(N(c4ccc(-c5ccc6c(c5)sc5ccccc56)cc4)c4ccc5ccc6ccccc6c5c4)c4c3oc3ccccc34)cc2)cc1 ; c1ccc(-c2ccc(-c3ccc(N(c4ccc(-c5cccc6c5sc5ccccc56)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccccc5)c5sc6ccccc6c45)c4c3oc3ccccc34)cc2)cc1] 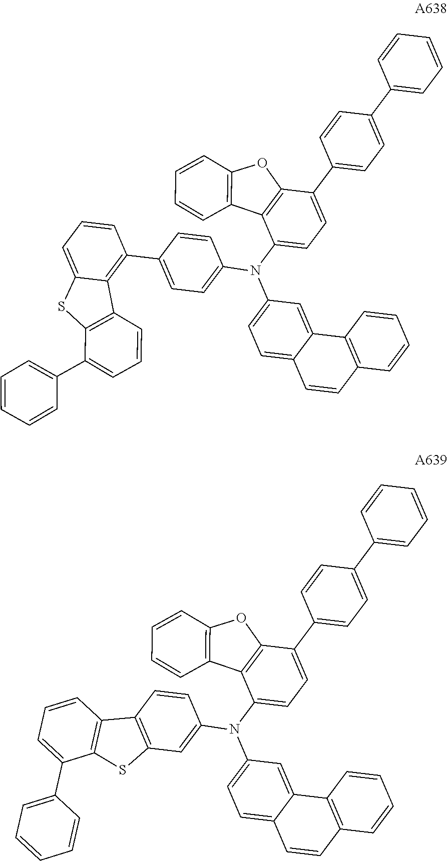 [SMILES: c1ccc(-c2ccc(-c3ccc(N(c4ccc(-c5cccc6sc7c(-c8ccccc8)cccc7c56)cc4)c4ccc5ccc6ccccc6c5c4)c4c3oc3ccccc34)cc2)cc1 ; c1ccc(-c2ccc(-c3ccc(N(c4ccc5c(c4)sc4c(-c6ccccc6)cccc45)c4ccc5ccc6ccccc6c5c4)c4c3oc3ccccc34)cc2)cc1] 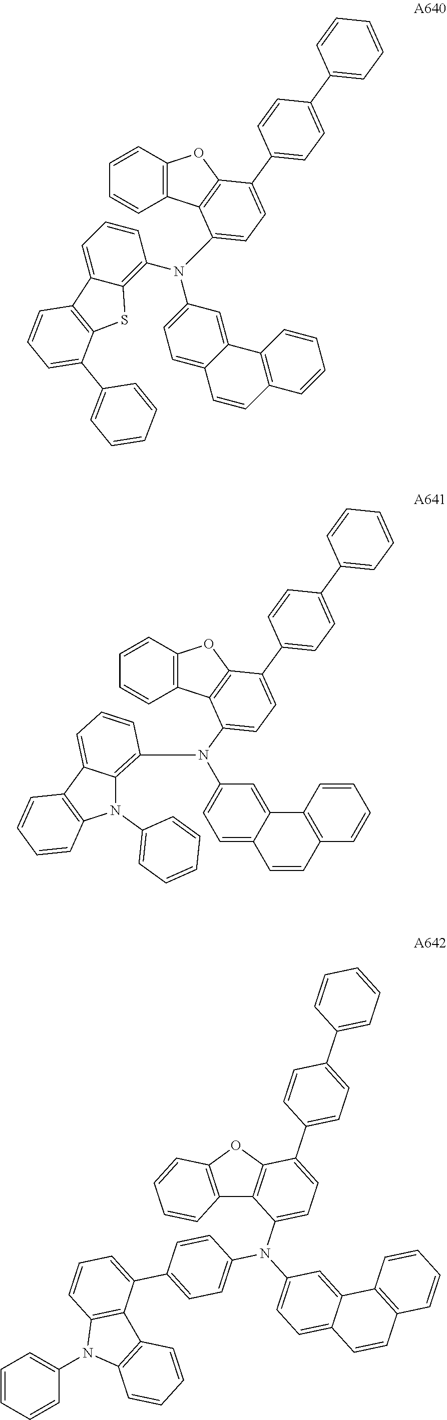 [SMILES: c1ccc(-c2ccc(-c3ccc(N(c4ccc5ccc6ccccc6c5c4)c4cccc5c4sc4c(-c6ccccc6)cccc45)c4c3oc3ccccc34)cc2)cc1 ; c1ccc(-c2ccc(-c3ccc(N(c4ccc5ccc6ccccc6c5c4)c4cccc5c6ccccc6n(-c6ccccc6)c45)c4c3oc3ccccc34)cc2)cc1 ; c1ccc(-c2ccc(-c3ccc(N(c4ccc(-c5cccc6c5c5ccccc5n6-c5ccccc5)cc4)c4ccc5ccc6ccccc6c5c4)c4c3oc3ccccc34)cc2)cc1] 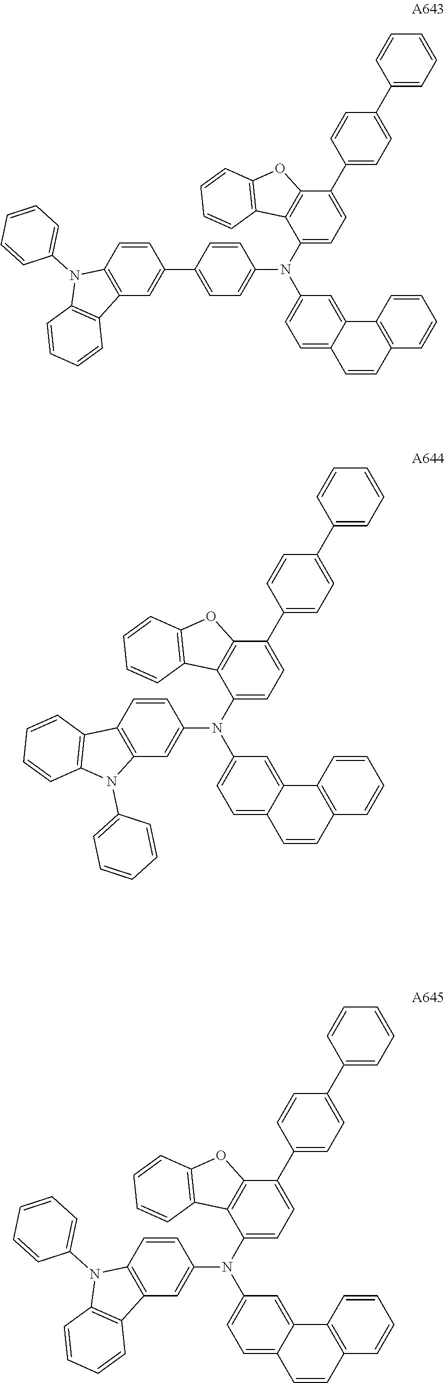 [SMILES: c1ccc(-c2ccc(-c3ccc(N(c4ccc(-c5ccc6c(c5)c5ccccc5n6-c5ccccc5)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc5c6ccccc6n(-c6ccccc6)c5c4)c4c3oc3ccccc34)cc2)cc1 ; c1ccc(-c2ccc(-c3ccc(N(c4ccc5ccc6ccccc6c5c4)c4ccc5c(c4)c4ccccc4n5-c4ccccc4)c4c3oc3ccccc34)cc2)cc1] 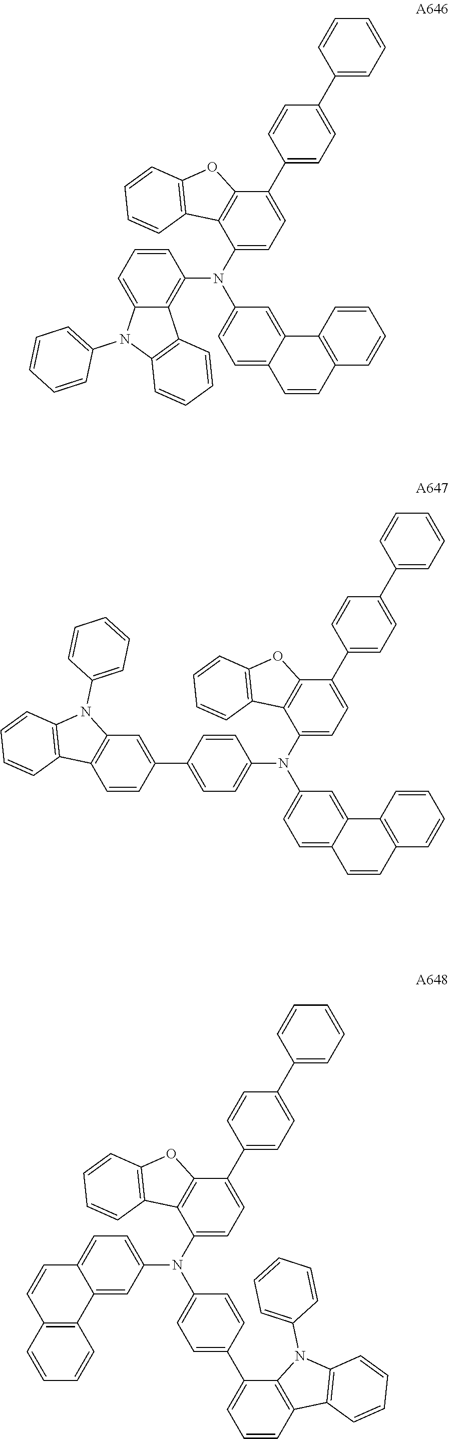 [SMILES: c1ccc(-c2ccc(-c3ccc(N(c4ccc5ccc6ccccc6c5c4)c4cccc5c4c4ccccc4n5-c4ccccc4)c4c3oc3ccccc34)cc2)cc1 ; c1ccc(-c2ccc(-c3ccc(N(c4ccc(-c5ccc6c7ccccc7n(-c7ccccc7)c6c5)cc4)c4ccc5ccc6ccccc6c5c4)c4c3oc3ccccc34)cc2)cc1 ; c1ccc(-c2ccc(-c3ccc(N(c4ccc(-c5cccc6c7ccccc7n(-c7ccccc7)c56)cc4)c4ccc5ccc6ccccc6c5c4)c4c3oc3ccccc34)cc2)cc1] 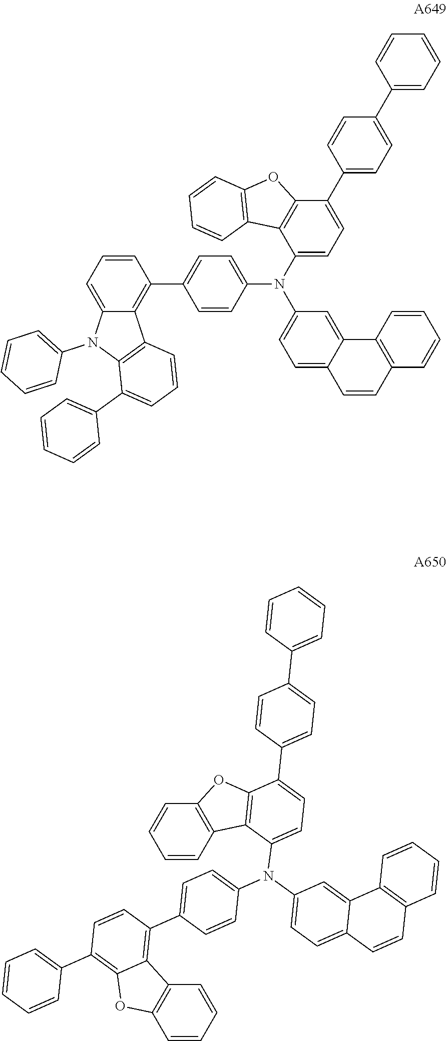 [SMILES: c1ccc(-c2ccc(-c3ccc(N(c4ccc(-c5cccc6c5c5cccc(-c7ccccc7)c5n6-c5ccccc5)cc4)c4ccc5ccc6ccccc6c5c4)c4c3oc3ccccc34)cc2)cc1 ; c1ccc(-c2ccc(-c3ccc(N(c4ccc(-c5ccc(-c6ccccc6)c6oc7ccccc7c56)cc4)c4ccc5ccc6ccccc6c5c4)c4c3oc3ccccc34)cc2)cc1] 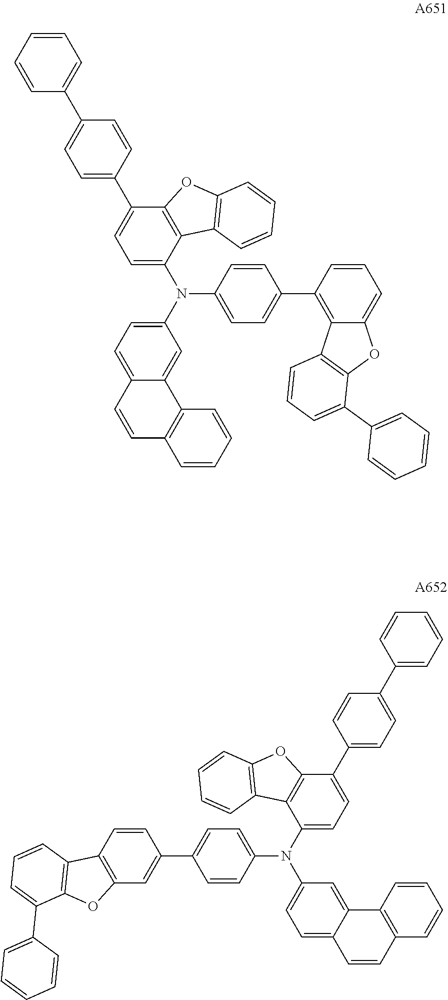 [SMILES: c1ccc(-c2ccc(-c3ccc(N(c4ccc(-c5cccc6oc7c(-c8ccccc8)cccc7c56)cc4)c4ccc5ccc6ccccc6c5c4)c4c3oc3ccccc34)cc2)cc1 ; c1ccc(-c2ccc(-c3ccc(N(c4ccc(-c5ccc6c(c5)oc5c(-c7ccccc7)cccc56)cc4)c4ccc5ccc6ccccc6c5c4)c4c3oc3ccccc34)cc2)cc1] 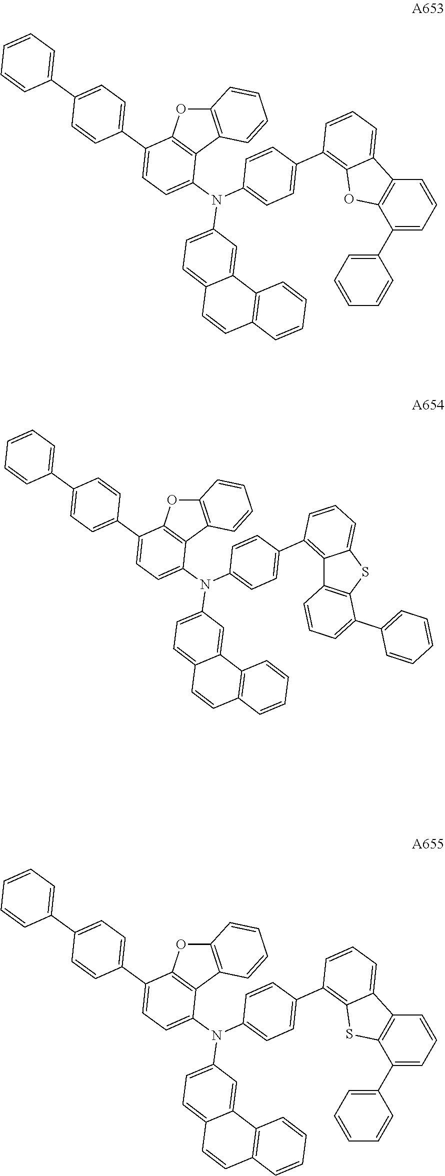 [SMILES: c1ccc(-c2ccc(-c3ccc(N(c4ccc(-c5cccc6c5oc5c(-c7ccccc7)cccc56)cc4)c4ccc5ccc6ccccc6c5c4)c4c3oc3ccccc34)cc2)cc1 ; c1ccc(-c2ccc(-c3ccc(N(c4ccc(-c5cccc6sc7c(-c8ccccc8)cccc7c56)cc4)c4ccc5ccc6ccccc6c5c4)c4c3oc3ccccc34)cc2)cc1 ; c1ccc(-c2ccc(-c3ccc(N(c4ccc(-c5cccc6c5sc5c(-c7ccccc7)cccc56)cc4)c4ccc5ccc6ccccc6c5c4)c4c3oc3ccccc34)cc2)cc1] 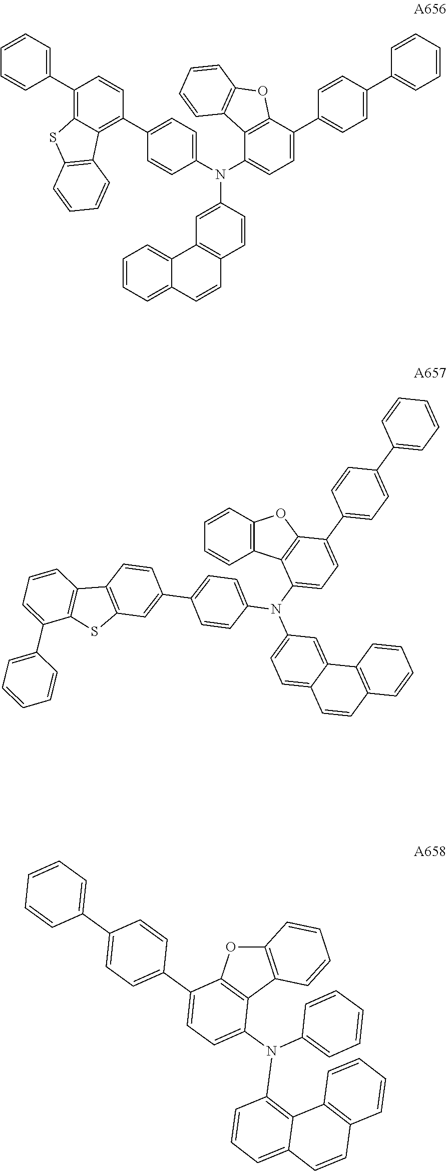 [SMILES: c1ccc(-c2ccc(-c3ccc(N(c4ccc(-c5ccc(-c6ccccc6)c6sc7ccccc7c56)cc4)c4ccc5ccc6ccccc6c5c4)c4c3oc3ccccc34)cc2)cc1 ; c1ccc(-c2ccc(-c3ccc(N(c4ccc(-c5ccc6c(c5)sc5c(-c7ccccc7)cccc56)cc4)c4ccc5ccc6ccccc6c5c4)c4c3oc3ccccc34)cc2)cc1 ; c1ccc(-c2ccc(-c3ccc(N(c4ccccc4)c4cccc5ccc6ccccc6c45)c4c3oc3ccccc34)cc2)cc1] 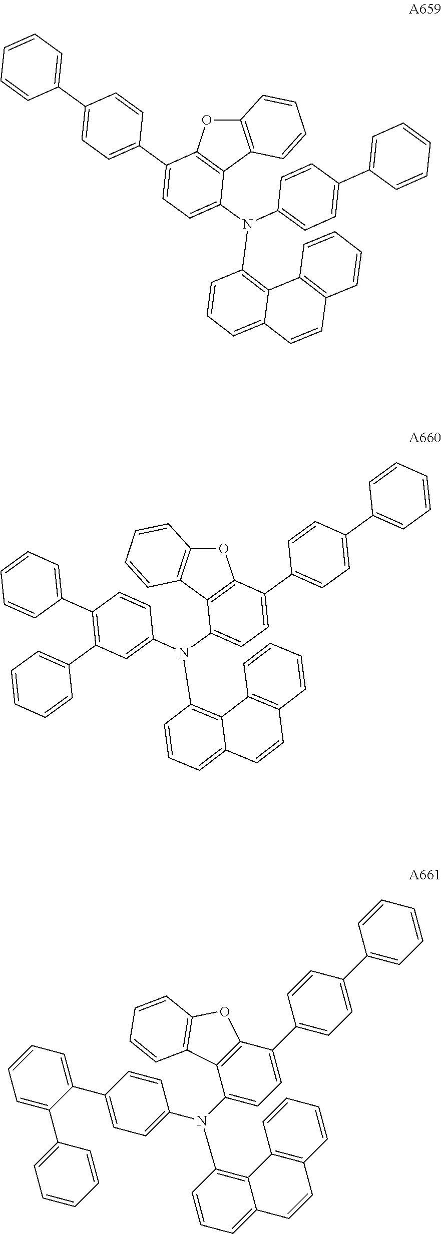 [SMILES: c1ccc(-c2ccc(-c3ccc(N(c4ccc(-c5ccccc5)cc4)c4cccc5ccc6ccccc6c45)c4c3oc3ccccc34)cc2)cc1 ; c1ccc(-c2ccc(-c3ccc(N(c4ccc(-c5ccccc5)c(-c5ccccc5)c4)c4cccc5ccc6ccccc6c45)c4c3oc3ccccc34)cc2)cc1 ; c1ccc(-c2ccc(-c3ccc(N(c4ccc(-c5ccccc5-c5ccccc5)cc4)c4cccc5ccc6ccccc6c45)c4c3oc3ccccc34)cc2)cc1] 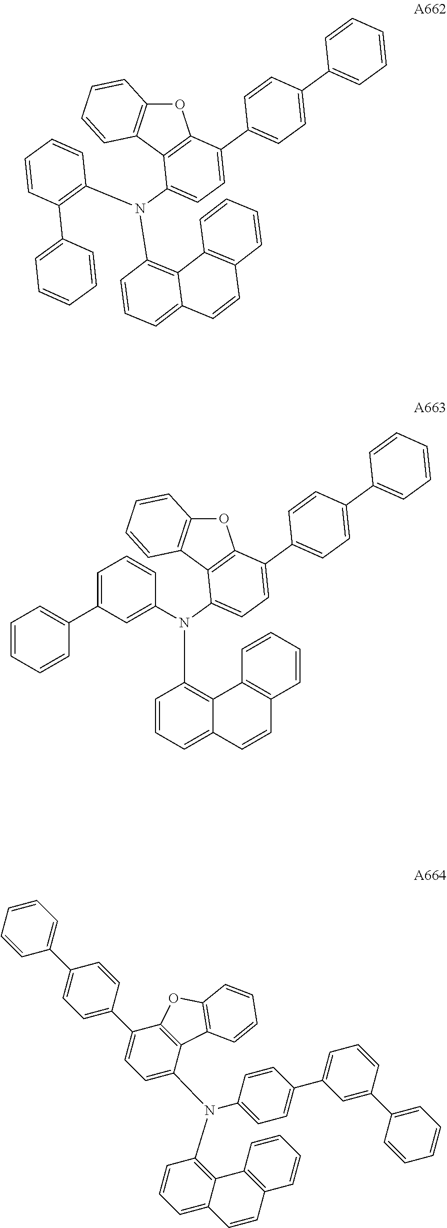 [SMILES: c1ccc(-c2ccc(-c3ccc(N(c4ccccc4-c4ccccc4)c4cccc5ccc6ccccc6c45)c4c3oc3ccccc34)cc2)cc1 ; c1ccc(-c2ccc(-c3ccc(N(c4cccc(-c5ccccc5)c4)c4cccc5ccc6ccccc6c45)c4c3oc3ccccc34)cc2)cc1 ; c1ccc(-c2ccc(-c3ccc(N(c4ccc(-c5cccc(-c6ccccc6)c5)cc4)c4cccc5ccc6ccccc6c45)c4c3oc3ccccc34)cc2)cc1] 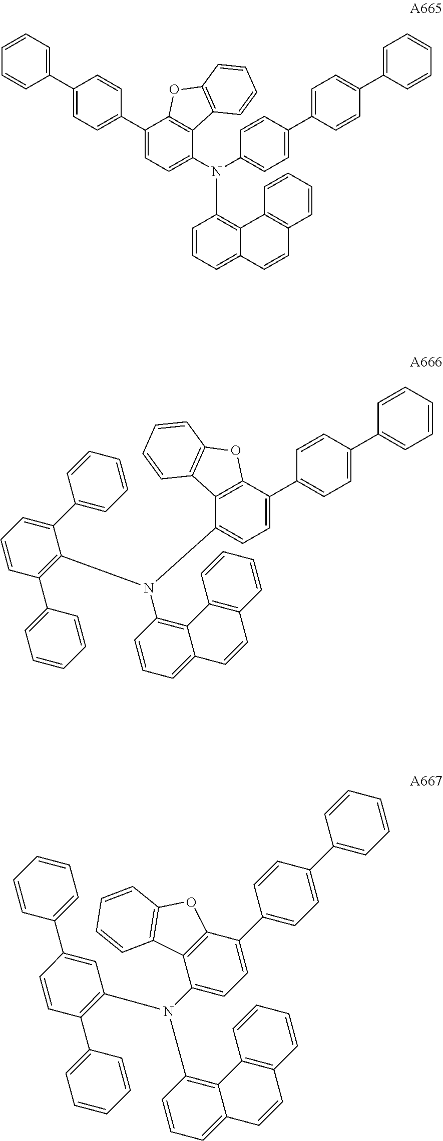 [SMILES: c1ccc(-c2ccc(-c3ccc(N(c4cccc5ccc6ccccc6c45)c4ccc(-c5ccc(-c6ccccc6)cc5)c5oc6ccccc6c45)cc3)cc2)cc1 ; c1ccc(-c2ccc(-c3ccc(N(c4c(-c5ccccc5)cccc4-c4ccccc4)c4cccc5ccc6ccccc6c45)c4c3oc3ccccc34)cc2)cc1 ; c1ccc(-c2ccc(-c3ccc(N(c4cc(-c5ccccc5)ccc4-c4ccccc4)c4cccc5ccc6ccccc6c45)c4c3oc3ccccc34)cc2)cc1] 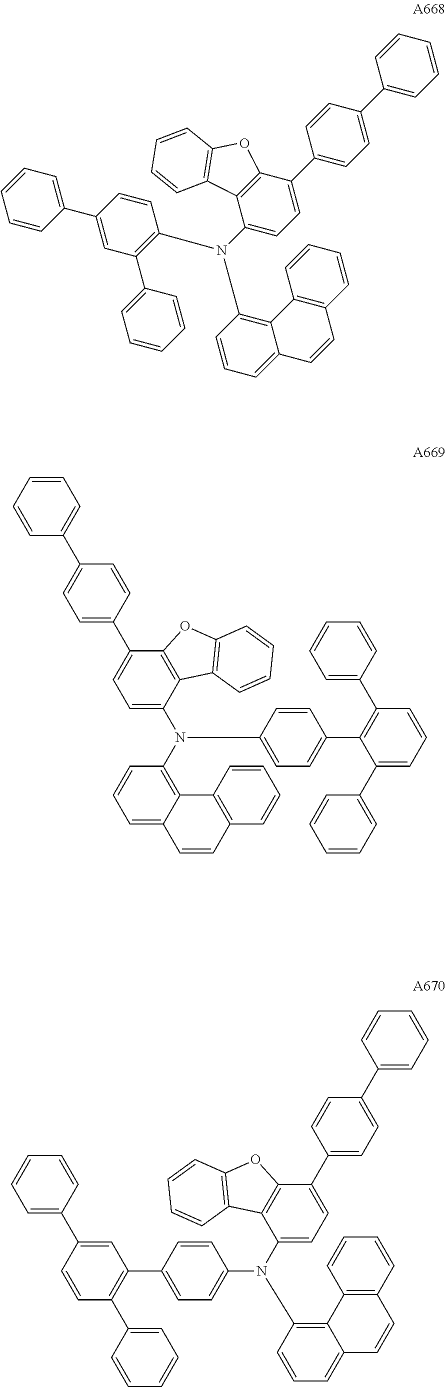 [SMILES: c1ccc(-c2ccc(-c3ccc(N(c4ccc(-c5ccccc5)cc4-c4ccccc4)c4cccc5ccc6ccccc6c45)c4c3oc3ccccc34)cc2)cc1 ; c1ccc(-c2ccc(-c3ccc(N(c4ccc(-c5c(-c6ccccc6)cccc5-c5ccccc5)cc4)c4cccc5ccc6ccccc6c45)c4c3oc3ccccc34)cc2)cc1 ; c1ccc(-c2ccc(-c3ccc(N(c4ccc(-c5cc(-c6ccccc6)ccc5-c5ccccc5)cc4)c4cccc5ccc6ccccc6c45)c4c3oc3ccccc34)cc2)cc1] 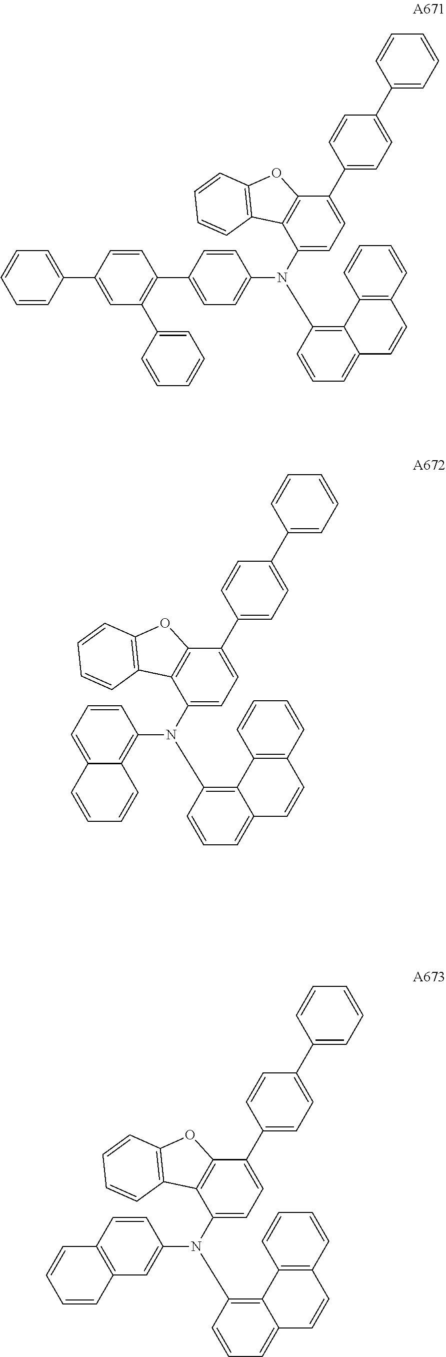 [SMILES: c1ccc(-c2ccc(-c3ccc(N(c4ccc(-c5ccc(-c6ccccc6)cc5-c5ccccc5)cc4)c4cccc5ccc6ccccc6c45)c4c3oc3ccccc34)cc2)cc1 ; c1ccc(-c2ccc(-c3ccc(N(c4cccc5ccccc45)c4cccc5ccc6ccccc6c45)c4c3oc3ccccc34)cc2)cc1 ; c1ccc(-c2ccc(-c3ccc(N(c4ccc5ccccc5c4)c4cccc5ccc6ccccc6c45)c4c3oc3ccccc34)cc2)cc1] 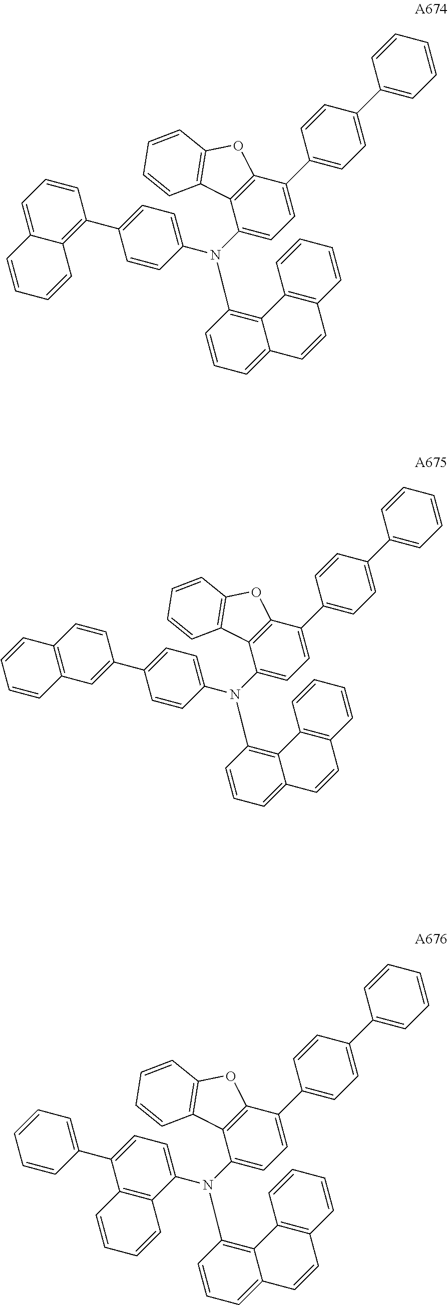 [SMILES: c1ccc(-c2ccc(-c3ccc(N(c4ccc(-c5cccc6ccccc56)cc4)c4cccc5ccc6ccccc6c45)c4c3oc3ccccc34)cc2)cc1 ; c1ccc(-c2ccc(-c3ccc(N(c4ccc(-c5ccc6ccccc6c5)cc4)c4cccc5ccc6ccccc6c45)c4c3oc3ccccc34)cc2)cc1 ; c1ccc(-c2ccc(-c3ccc(N(c4ccc(-c5ccccc5)c5ccccc45)c4cccc5ccc6ccccc6c45)c4c3oc3ccccc34)cc2)cc1] 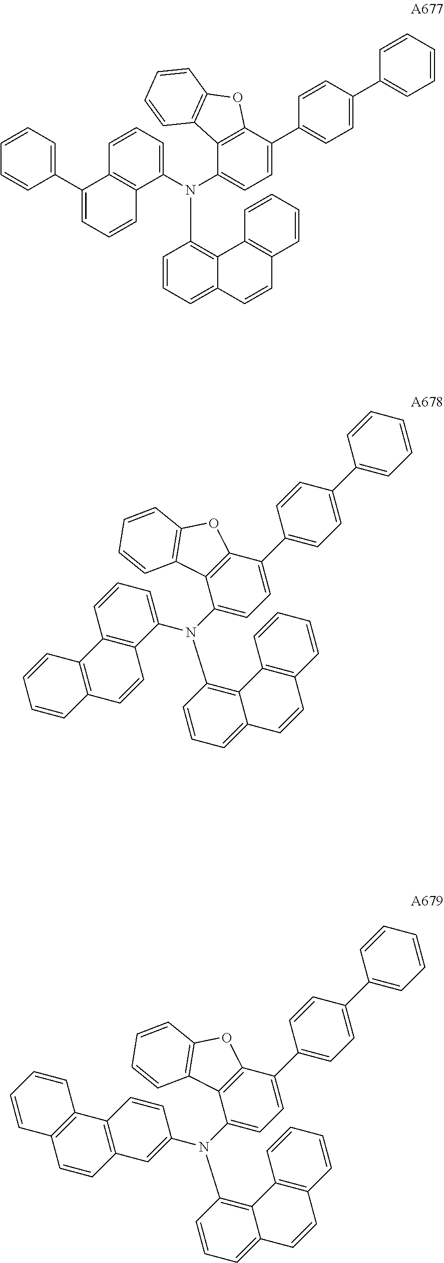 [SMILES: c1ccc(-c2ccc(-c3ccc(N(c4cccc5c(-c6ccccc6)cccc45)c4cccc5ccc6ccccc6c45)c4c3oc3ccccc34)cc2)cc1 ; c1ccc(-c2ccc(-c3ccc(N(c4cccc5c4ccc4ccccc45)c4cccc5ccc6ccccc6c45)c4c3oc3ccccc34)cc2)cc1 ; c1ccc(-c2ccc(-c3ccc(N(c4ccc5c(ccc6ccccc65)c4)c4cccc5ccc6ccccc6c45)c4c3oc3ccccc34)cc2)cc1] 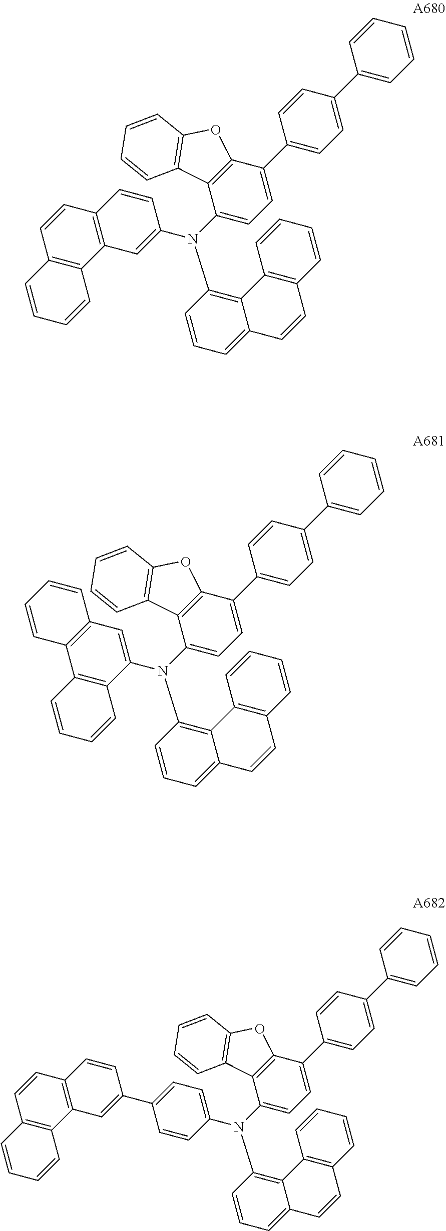 [SMILES: c1ccc(-c2ccc(-c3ccc(N(c4ccc5ccc6ccccc6c5c4)c4cccc5ccc6ccccc6c45)c4c3oc3ccccc34)cc2)cc1 ; c1ccc(-c2ccc(-c3ccc(N(c4cc5ccccc5c5ccccc45)c4cccc5ccc6ccccc6c45)c4c3oc3ccccc34)cc2)cc1 ; c1ccc(-c2ccc(-c3ccc(N(c4ccc(-c5ccc6ccc7ccccc7c6c5)cc4)c4cccc5ccc6ccccc6c45)c4c3oc3ccccc34)cc2)cc1] 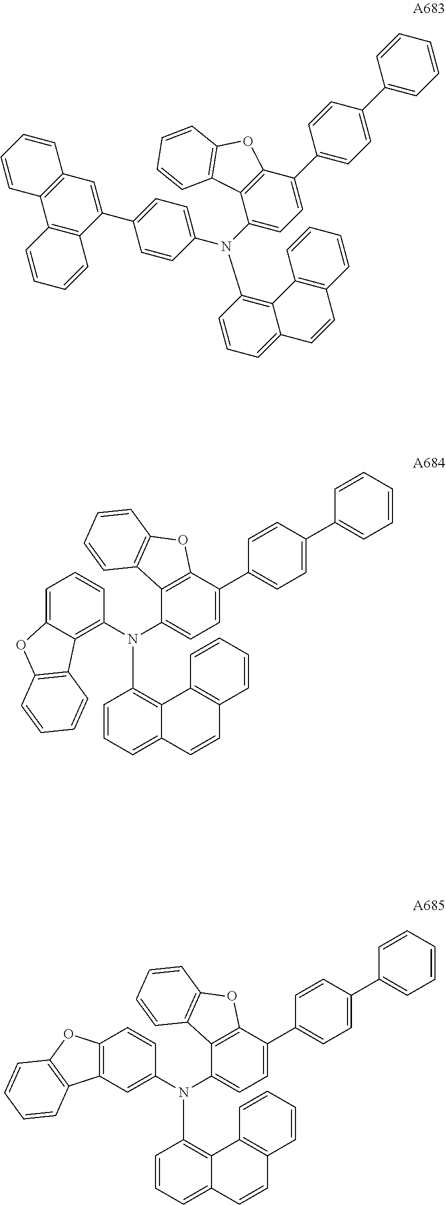 [SMILES: c1ccc(-c2ccc(-c3ccc(N(c4ccc(-c5cc6ccccc6c6ccccc56)cc4)c4cccc5ccc6ccccc6c45)c4c3oc3ccccc34)cc2)cc1 ; c1ccc(-c2ccc(-c3ccc(N(c4cccc5ccc6ccccc6c45)c4cccc5oc6ccccc6c45)c4c3oc3ccccc34)cc2)cc1 ; c1ccc(-c2ccc(-c3ccc(N(c4ccc5oc6ccccc6c5c4)c4cccc5ccc6ccccc6c45)c4c3oc3ccccc34)cc2)cc1] 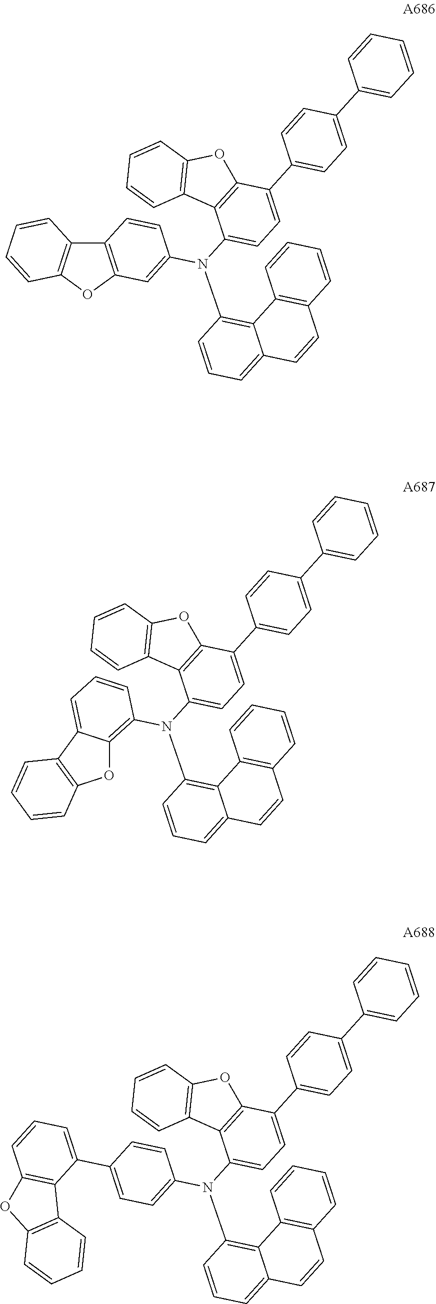 [SMILES: c1ccc(-c2ccc(-c3ccc(N(c4ccc5c(c4)oc4ccccc45)c4cccc5ccc6ccccc6c45)c4c3oc3ccccc34)cc2)cc1 ; c1ccc(-c2ccc(-c3ccc(N(c4cccc5c4oc4ccccc45)c4cccc5ccc6ccccc6c45)c4c3oc3ccccc34)cc2)cc1 ; c1ccc(-c2ccc(-c3ccc(N(c4ccc(-c5cccc6oc7ccccc7c56)cc4)c4cccc5ccc6ccccc6c45)c4c3oc3ccccc34)cc2)cc1] 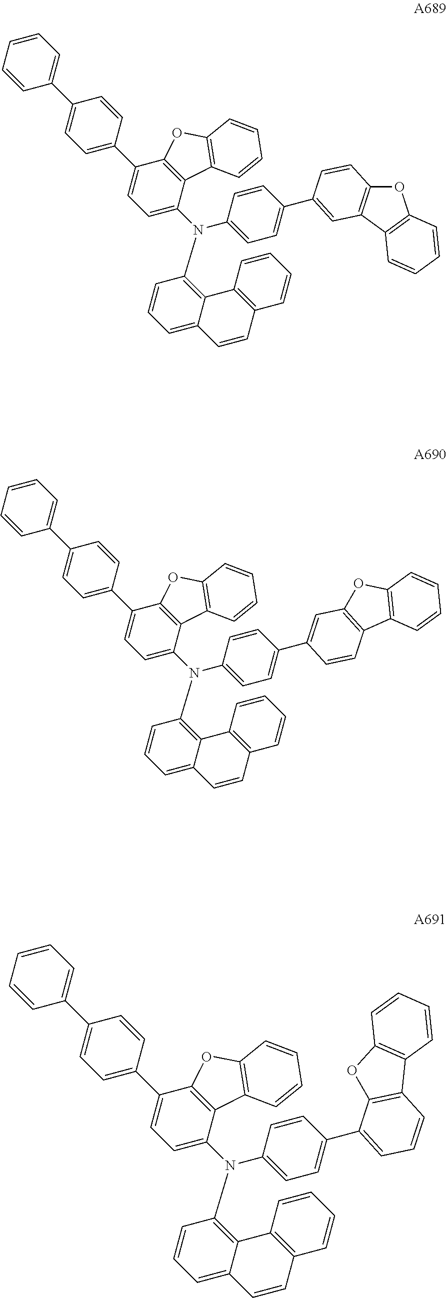 [SMILES: c1ccc(-c2ccc(-c3ccc(N(c4ccc(-c5ccc6oc7ccccc7c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)oc5ccccc56)cc4)c4cccc5ccc6ccccc6c45)c4c3oc3ccccc34)cc2)cc1 ; c1ccc(-c2ccc(-c3ccc(N(c4ccc(-c5cccc6c5oc5ccccc56)cc4)c4cccc5ccc6ccccc6c45)c4c3oc3ccccc34)cc2)cc1] 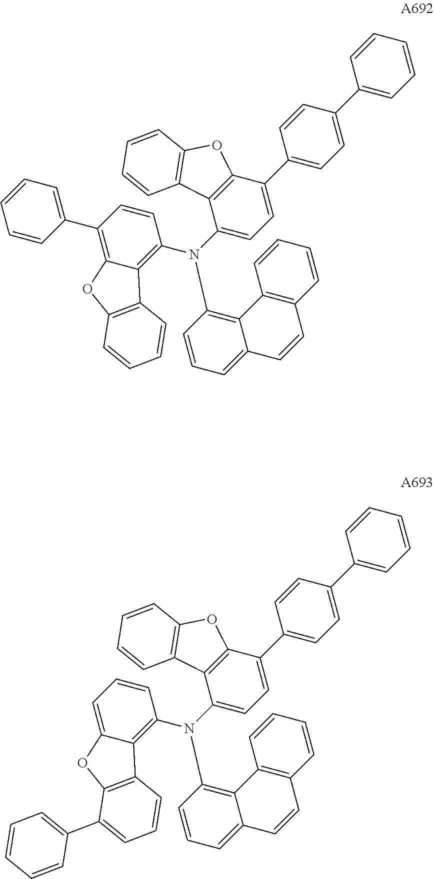 [SMILES: c1ccc(-c2ccc(-c3ccc(N(c4cccc5ccc6ccccc6c45)c4ccc(-c5ccccc5)c5oc6ccccc6c45)c4c3oc3ccccc34)cc2)cc1 ; c1ccc(-c2ccc(-c3ccc(N(c4cccc5ccc6ccccc6c45)c4cccc5oc6c(-c7ccccc7)cccc6c45)c4c3oc3ccccc34)cc2)cc1] 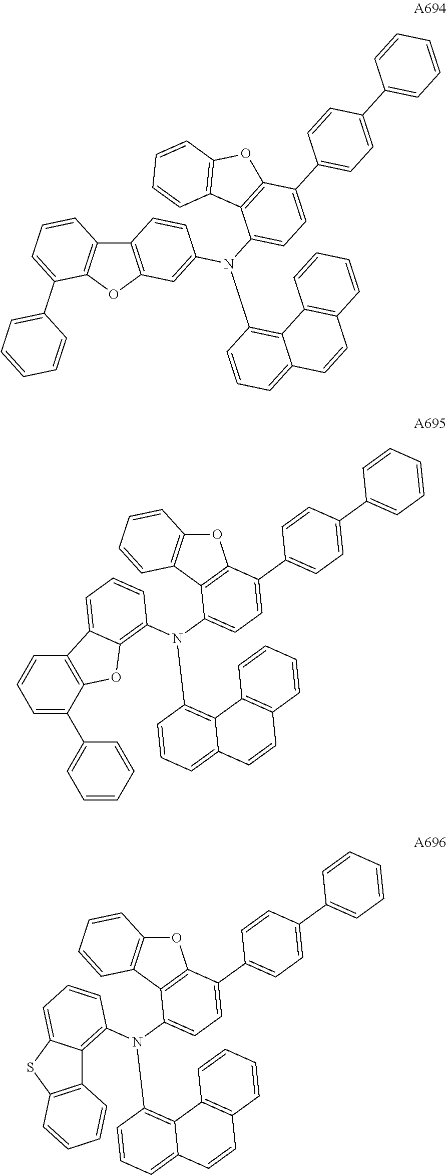 [SMILES: c1ccc(-c2ccc(-c3ccc(N(c4ccc5c(c4)oc4c(-c6ccccc6)cccc45)c4cccc5ccc6ccccc6c45)c4c3oc3ccccc34)cc2)cc1 ; c1ccc(-c2ccc(-c3ccc(N(c4cccc5c4oc4c(-c6ccccc6)cccc45)c4cccc5ccc6ccccc6c45)c4c3oc3ccccc34)cc2)cc1 ; c1ccc(-c2ccc(-c3ccc(N(c4cccc5ccc6ccccc6c45)c4cccc5sc6ccccc6c45)c4c3oc3ccccc34)cc2)cc1] 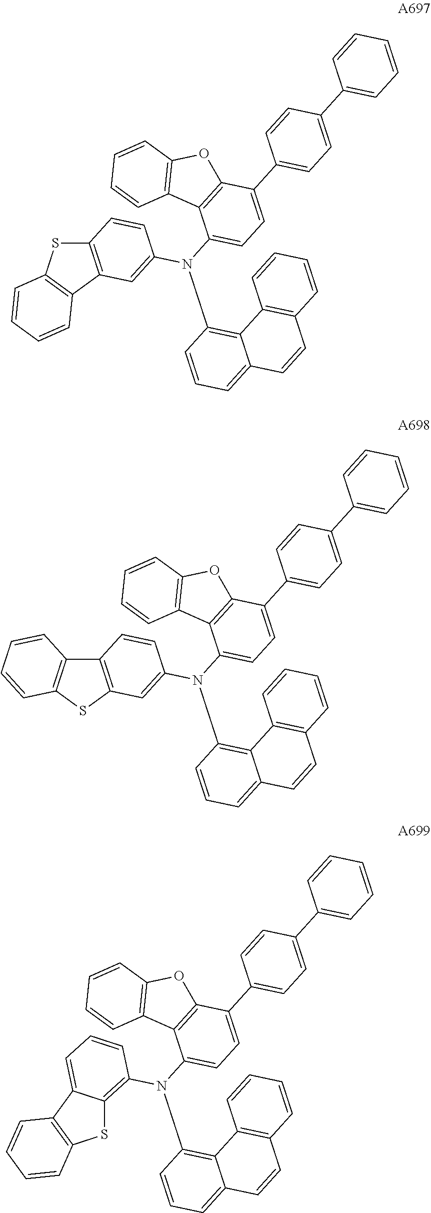 [SMILES: c1ccc(-c2ccc(-c3ccc(N(c4ccc5sc6ccccc6c5c4)c4cccc5ccc6ccccc6c45)c4c3oc3ccccc34)cc2)cc1 ; c1ccc(-c2ccc(-c3ccc(N(c4ccc5c(c4)sc4ccccc45)c4cccc5ccc6ccccc6c45)c4c3oc3ccccc34)cc2)cc1 ; c1ccc(-c2ccc(-c3ccc(N(c4cccc5c4sc4ccccc45)c4cccc5ccc6ccccc6c45)c4c3oc3ccccc34)cc2)cc1] 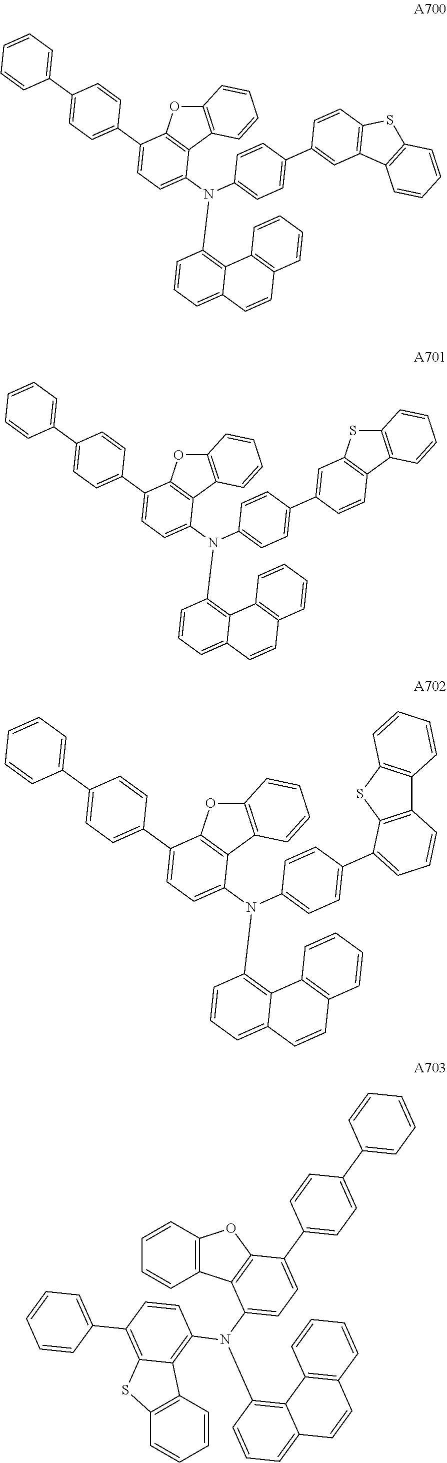 [SMILES: c1ccc(-c2ccc(-c3ccc(N(c4ccc(-c5ccc6sc7ccccc7c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)sc5ccccc56)cc4)c4cccc5ccc6ccccc6c45)c4c3oc3ccccc34)cc2)cc1 ; c1ccc(-c2ccc(-c3ccc(N(c4ccc(-c5cccc6c5sc5ccccc56)cc4)c4cccc5ccc6ccccc6c45)c4c3oc3ccccc34)cc2)cc1 ; c1ccc(-c2ccc(-c3ccc(N(c4cccc5ccc6ccccc6c45)c4ccc(-c5ccccc5)c5sc6ccccc6c45)c4c3oc3ccccc34)cc2)cc1] 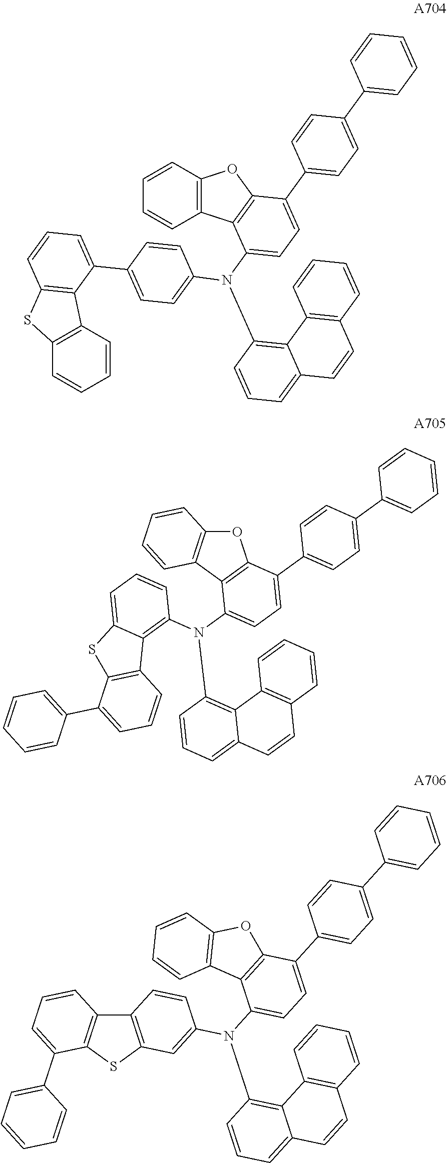 [SMILES: c1ccc(-c2ccc(-c3ccc(N(c4ccc(-c5cccc6sc7ccccc7c56)cc4)c4cccc5ccc6ccccc6c45)c4c3oc3ccccc34)cc2)cc1 ; c1ccc(-c2ccc(-c3ccc(N(c4cccc5ccc6ccccc6c45)c4cccc5sc6c(-c7ccccc7)cccc6c45)c4c3oc3ccccc34)cc2)cc1 ; c1ccc(-c2ccc(-c3ccc(N(c4ccc5c(c4)sc4c(-c6ccccc6)cccc45)c4cccc5ccc6ccccc6c45)c4c3oc3ccccc34)cc2)cc1] 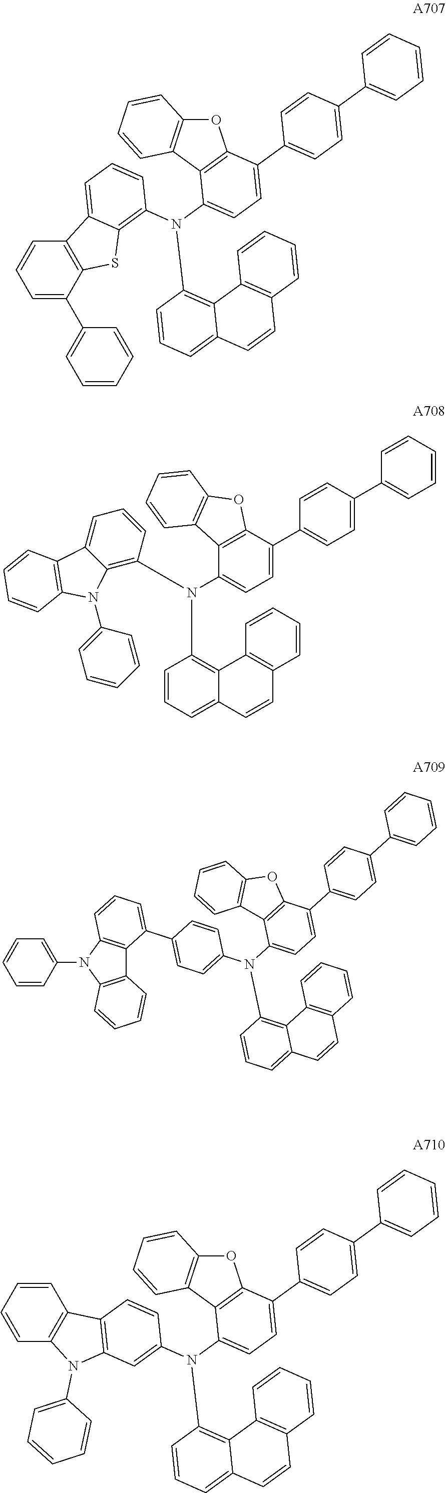 [SMILES: c1ccc(-c2ccc(-c3ccc(N(c4cccc5c4sc4c(-c6ccccc6)cccc45)c4cccc5ccc6ccccc6c45)c4c3oc3ccccc34)cc2)cc1 ; c1ccc(-c2ccc(-c3ccc(N(c4cccc5ccc6ccccc6c45)c4cccc5c6ccccc6n(-c6ccccc6)c45)c4c3oc3ccccc34)cc2)cc1 ; c1ccc(-c2ccc(-c3ccc(N(c4ccc(-c5cccc6c5c5ccccc5n6-c5ccccc5)cc4)c4cccc5ccc6ccccc6c45)c4c3oc3ccccc34)cc2)cc1 ; c1ccc(-c2ccc(-c3ccc(N(c4ccc5c6ccccc6n(-c6ccccc6)c5c4)c4cccc5ccc6ccccc6c45)c4c3oc3ccccc34)cc2)cc1] 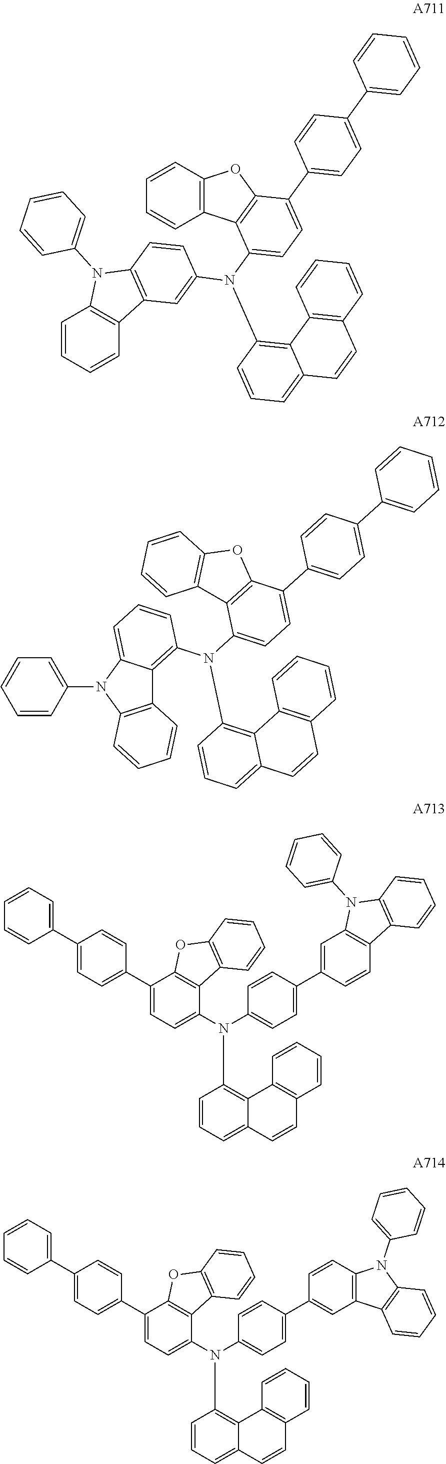 [SMILES: c1ccc(-c2ccc(-c3ccc(N(c4ccc5c(c4)c4ccccc4n5-c4ccccc4)c4cccc5ccc6ccccc6c45)c4c3oc3ccccc34)cc2)cc1 ; c1ccc(-c2ccc(-c3ccc(N(c4cccc5ccc6ccccc6c45)c4cccc5c4c4ccccc4n5-c4ccccc4)c4c3oc3ccccc34)cc2)cc1 ; c1ccc(-c2ccc(-c3ccc(N(c4ccc(-c5ccc6c7ccccc7n(-c7ccccc7)c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)c5ccccc5n6-c5ccccc5)cc4)c4cccc5ccc6ccccc6c45)c4c3oc3ccccc34)cc2)cc1] 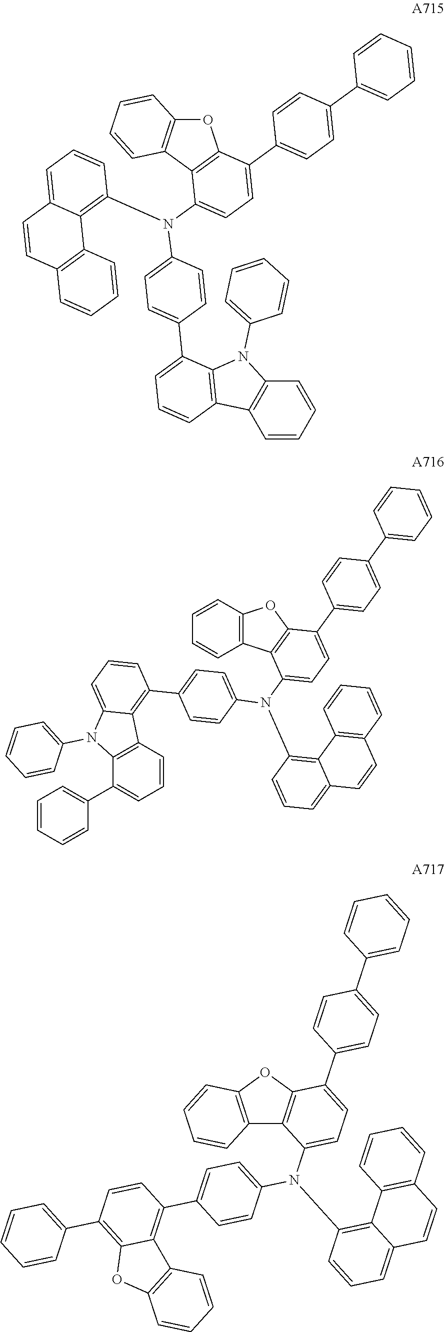 [SMILES: c1ccc(-c2ccc(-c3ccc(N(c4ccc(-c5cccc6c7ccccc7n(-c7ccccc7)c56)cc4)c4cccc5ccc6ccccc6c45)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4cccc5ccc6ccccc6c45)c4c3oc3ccccc34)cc2)cc1 ; c1ccc(-c2ccc(-c3ccc(N(c4ccc(-c5ccc(-c6ccccc6)c6oc7ccccc7c56)cc4)c4cccc5ccc6ccccc6c45)c4c3oc3ccccc34)cc2)cc1] 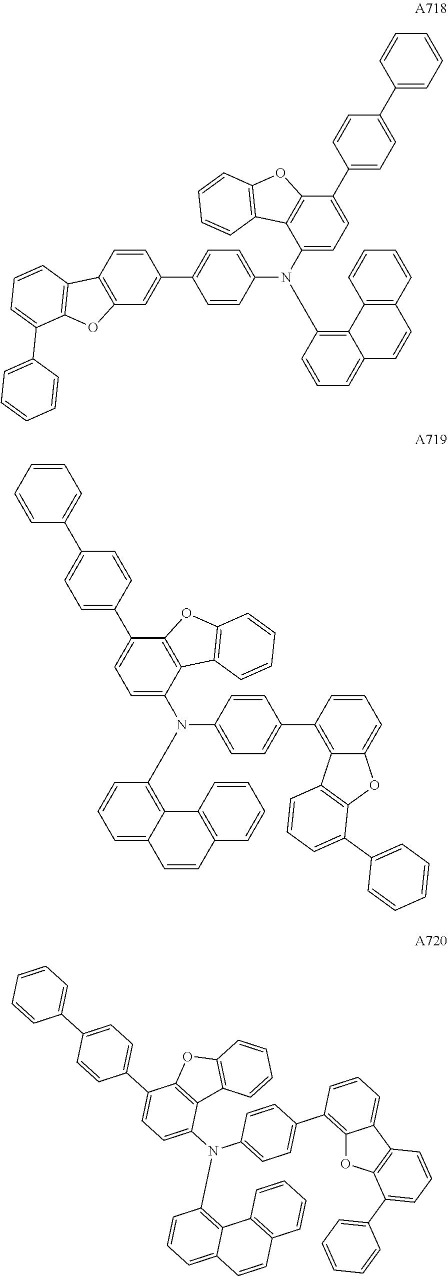 [SMILES: c1ccc(-c2ccc(-c3ccc(N(c4ccc(-c5ccc6c(c5)oc5c(-c7ccccc7)cccc56)cc4)c4cccc5ccc6ccccc6c45)c4c3oc3ccccc34)cc2)cc1 ; c1ccc(-c2ccc(-c3ccc(N(c4ccc(-c5cccc6oc7c(-c8ccccc8)cccc7c56)cc4)c4cccc5ccc6ccccc6c45)c4c3oc3ccccc34)cc2)cc1 ; c1ccc(-c2ccc(-c3ccc(N(c4ccc(-c5cccc6c5oc5c(-c7ccccc7)cccc56)cc4)c4cccc5ccc6ccccc6c45)c4c3oc3ccccc34)cc2)cc1] 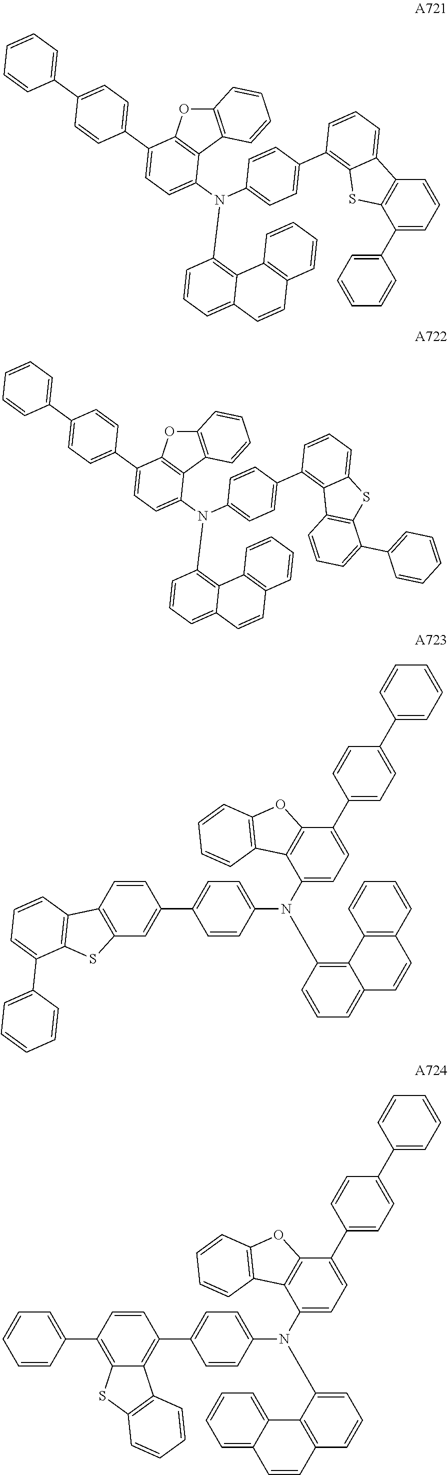 [SMILES: c1ccc(-c2ccc(-c3ccc(N(c4ccc(-c5cccc6c5sc5c(-c7ccccc7)cccc56)cc4)c4cccc5ccc6ccccc6c45)c4c3oc3ccccc34)cc2)cc1 ; c1ccc(-c2ccc(-c3ccc(N(c4ccc(-c5cccc6sc7c(-c8ccccc8)cccc7c56)cc4)c4cccc5ccc6ccccc6c45)c4c3oc3ccccc34)cc2)cc1 ; c1ccc(-c2ccc(-c3ccc(N(c4ccc(-c5ccc6c(c5)sc5c(-c7ccccc7)cccc56)cc4)c4cccc5ccc6ccccc6c45)c4c3oc3ccccc34)cc2)cc1 ; c1ccc(-c2ccc(-c3ccc(N(c4ccc(-c5ccc(-c6ccccc6)c6sc7ccccc7c56)cc4)c4cccc5ccc6ccccc6c45)c4c3oc3ccccc34)cc2)cc1] 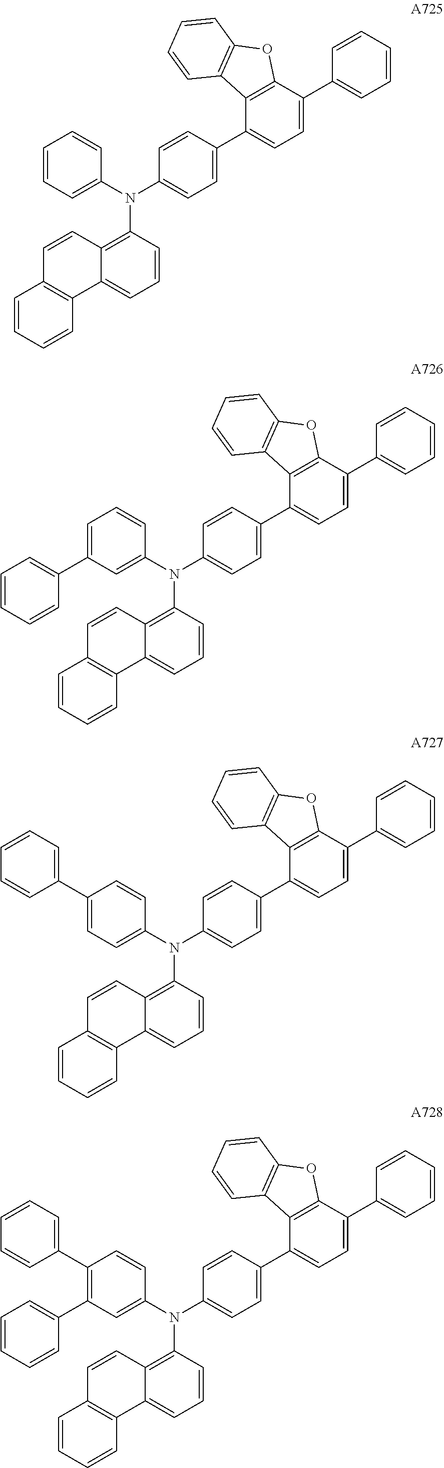 [SMILES: c1ccc(-c2ccc(-c3ccc(N(c4ccccc4)c4cccc5c4ccc4ccccc45)cc3)c3c2oc2ccccc23)cc1 ; c1ccc(-c2cccc(N(c3ccc(-c4ccc(-c5ccccc5)c5oc6ccccc6c45)cc3)c3cccc4c3ccc3ccccc34)c2)cc1 ; c1ccc(-c2ccc(N(c3ccc(-c4ccc(-c5ccccc5)c5oc6ccccc6c45)cc3)c3cccc4c3ccc3ccccc34)cc2)cc1 ; c1ccc(-c2ccc(N(c3ccc(-c4ccc(-c5ccccc5)c5oc6ccccc6c45)cc3)c3cccc4c3ccc3ccccc34)cc2-c2ccccc2)cc1] 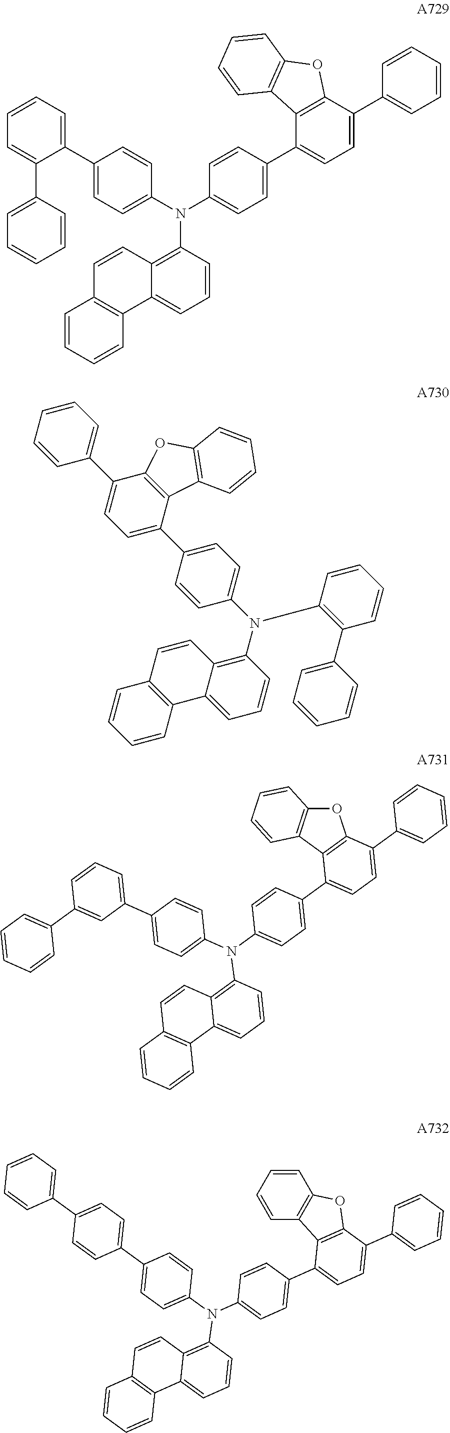 [SMILES: c1ccc(-c2ccccc2-c2ccc(N(c3ccc(-c4ccc(-c5ccccc5)c5oc6ccccc6c45)cc3)c3cccc4c3ccc3ccccc34)cc2)cc1 ; c1ccc(-c2ccccc2N(c2ccc(-c3ccc(-c4ccccc4)c4oc5ccccc5c34)cc2)c2cccc3c2ccc2ccccc23)cc1 ; c1ccc(-c2cccc(-c3ccc(N(c4ccc(-c5ccc(-c6ccccc6)c6oc7ccccc7c56)cc4)c4cccc5c4ccc4ccccc45)cc3)c2)cc1 ; c1ccc(-c2ccc(-c3ccc(N(c4ccc(-c5ccc(-c6ccccc6)c6oc7ccccc7c56)cc4)c4cccc5c4ccc4ccccc45)cc3)cc2)cc1] 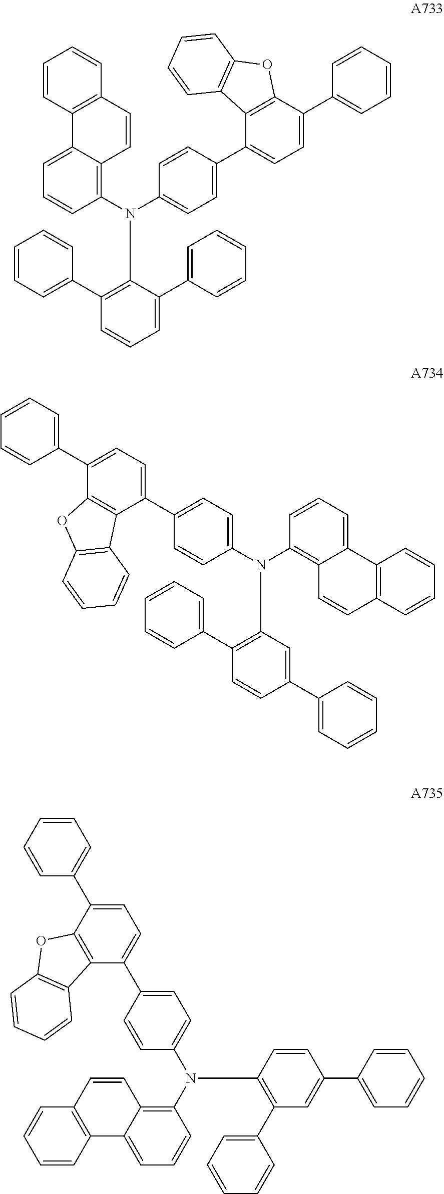 [SMILES: c1ccc(-c2cccc(-c3ccccc3)c2N(c2ccc(-c3ccc(-c4ccccc4)c4oc5ccccc5c34)cc2)c2cccc3c2ccc2ccccc23)cc1 ; c1ccc(-c2ccc(-c3ccccc3)c(N(c3ccc(-c4ccc(-c5ccccc5)c5oc6ccccc6c45)cc3)c3cccc4c3ccc3ccccc34)c2)cc1 ; c1ccc(-c2ccc(N(c3ccc(-c4ccc(-c5ccccc5)c5oc6ccccc6c45)cc3)c3cccc4c3ccc3ccccc34)c(-c3ccccc3)c2)cc1] 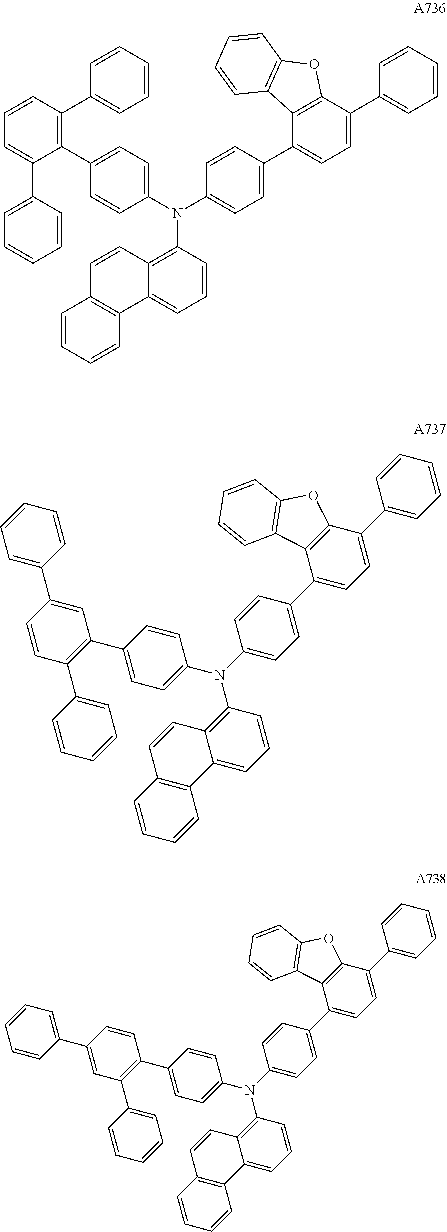 [SMILES: c1ccc(-c2cccc(-c3ccccc3)c2-c2ccc(N(c3ccc(-c4ccc(-c5ccccc5)c5oc6ccccc6c45)cc3)c3cccc4c3ccc3ccccc34)cc2)cc1 ; c1ccc(-c2ccc(-c3ccccc3)c(-c3ccc(N(c4ccc(-c5ccc(-c6ccccc6)c6oc7ccccc7c56)cc4)c4cccc5c4ccc4ccccc45)cc3)c2)cc1 ; c1ccc(-c2ccc(-c3ccc(N(c4ccc(-c5ccc(-c6ccccc6)c6oc7ccccc7c56)cc4)c4cccc5c4ccc4ccccc45)cc3)c(-c3ccccc3)c2)cc1] 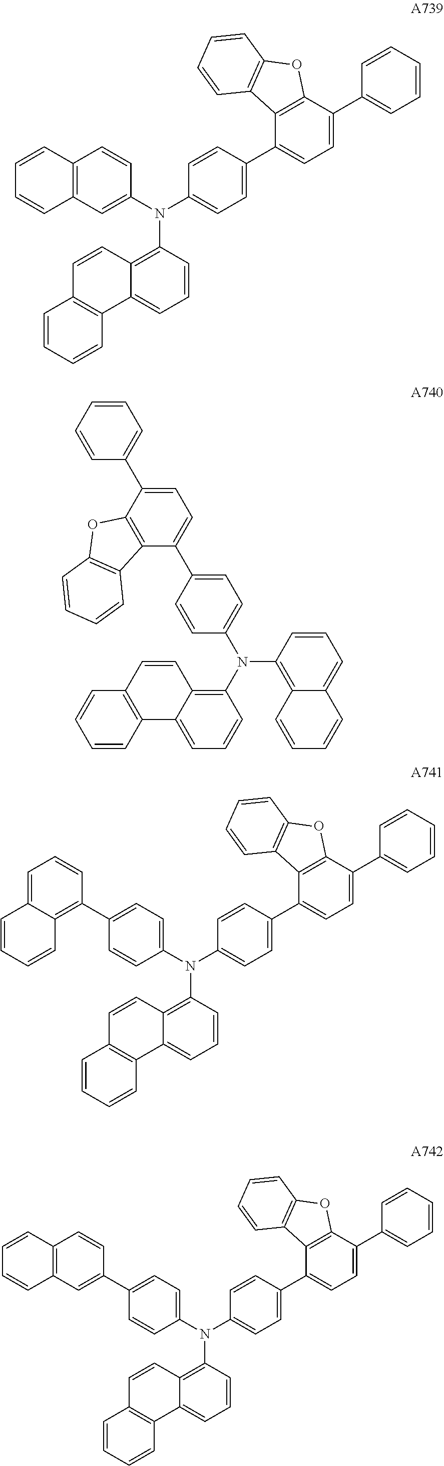 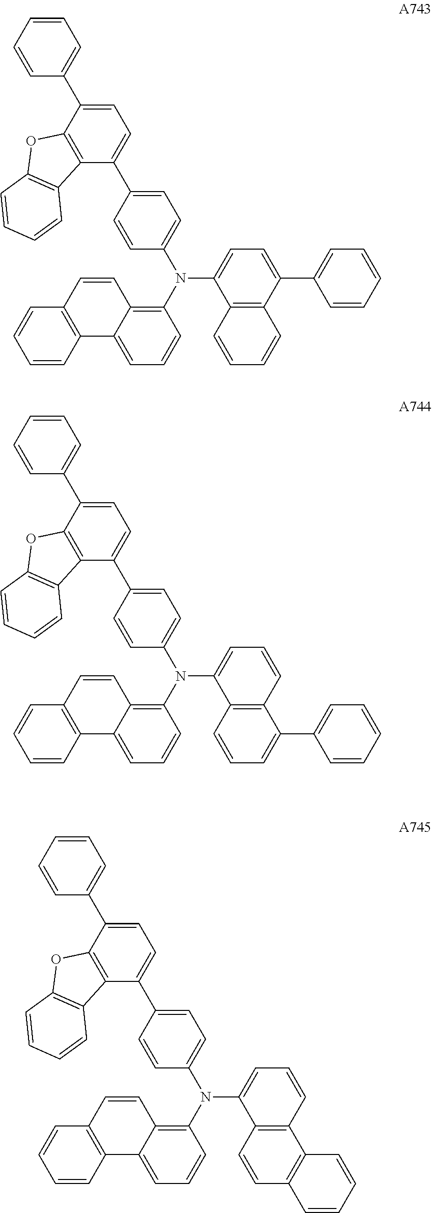 [SMILES: c1ccc(-c2ccc(N(c3ccc(-c4ccc(-c5ccccc5)c5oc6ccccc6c45)cc3)c3cccc4c3ccc3ccccc34)c3ccccc23)cc1 ; c1ccc(-c2cccc3c(N(c4ccc(-c5ccc(-c6ccccc6)c6oc7ccccc7c56)cc4)c4cccc5c4ccc4ccccc45)cccc23)cc1 ; c1ccc(-c2ccc(-c3ccc(N(c4cccc5c4ccc4ccccc45)c4cccc5c4ccc4ccccc45)cc3)c3c2oc2ccccc23)cc1] 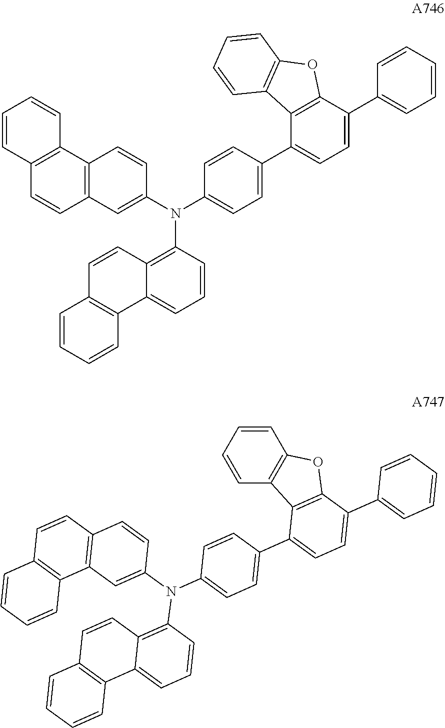 [SMILES: c1ccc(-c2ccc(-c3ccc(N(c4ccc5c(ccc6ccccc65)c4)c4cccc5c4ccc4ccccc45)cc3)c3c2oc2ccccc23)cc1 ; c1ccc(-c2ccc(-c3ccc(N(c4ccc5ccc6ccccc6c5c4)c4cccc5c4ccc4ccccc45)cc3)c3c2oc2ccccc23)cc1] 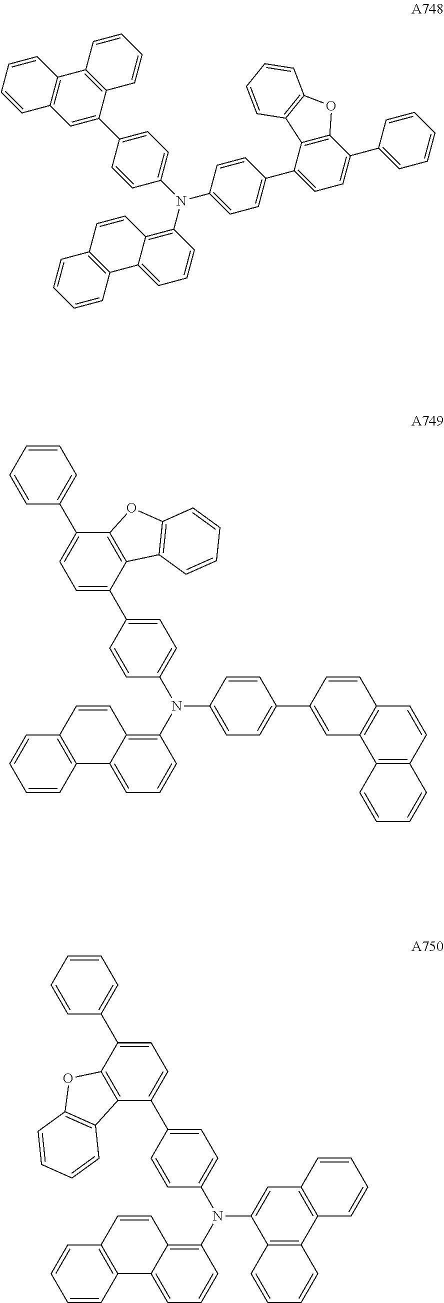 [SMILES: c1ccc(-c2ccc(-c3ccc(N(c4ccc(-c5cc6ccccc6c6ccccc56)cc4)c4cccc5c4ccc4ccccc45)cc3)c3c2oc2ccccc23)cc1 ; c1ccc(-c2ccc(-c3ccc(N(c4ccc(-c5ccc6ccc7ccccc7c6c5)cc4)c4cccc5c4ccc4ccccc45)cc3)c3c2oc2ccccc23)cc1 ; c1ccc(-c2ccc(-c3ccc(N(c4cccc5c4ccc4ccccc45)c4cc5ccccc5c5ccccc45)cc3)c3c2oc2ccccc23)cc1] 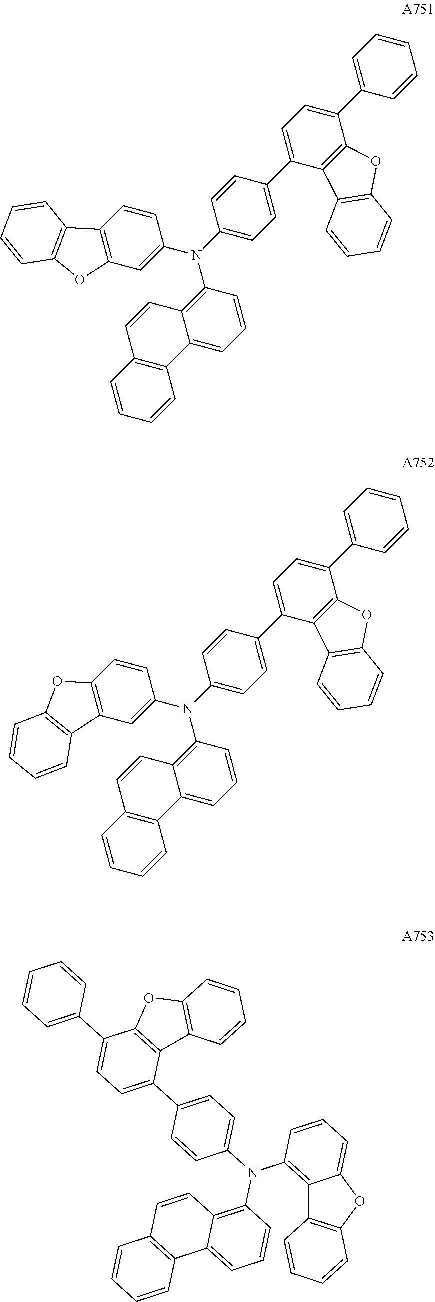 [SMILES: c1ccc(-c2ccc(-c3ccc(N(c4ccc5c(c4)oc4ccccc45)c4cccc5c4ccc4ccccc45)cc3)c3c2oc2ccccc23)cc1 ; c1ccc(-c2ccc(-c3ccc(N(c4ccc5oc6ccccc6c5c4)c4cccc5c4ccc4ccccc45)cc3)c3c2oc2ccccc23)cc1 ; c1ccc(-c2ccc(-c3ccc(N(c4cccc5c4ccc4ccccc45)c4cccc5oc6ccccc6c45)cc3)c3c2oc2ccccc23)cc1] 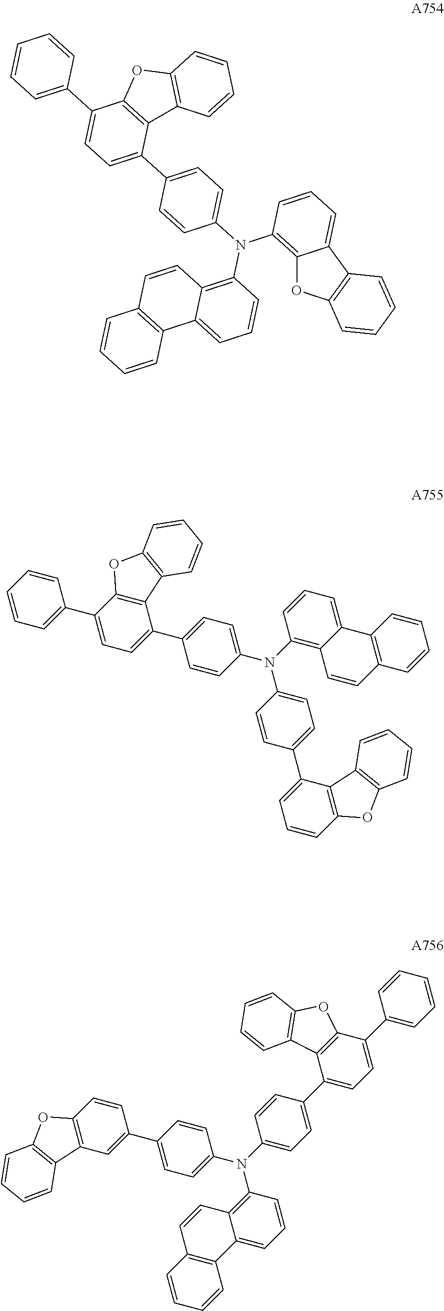 [SMILES: c1ccc(-c2ccc(-c3ccc(N(c4cccc5c4ccc4ccccc45)c4cccc5c4oc4ccccc45)cc3)c3c2oc2ccccc23)cc1 ; c1ccc(-c2ccc(-c3ccc(N(c4ccc(-c5cccc6oc7ccccc7c56)cc4)c4cccc5c4ccc4ccccc45)cc3)c3c2oc2ccccc23)cc1 ; c1ccc(-c2ccc(-c3ccc(N(c4ccc(-c5ccc6oc7ccccc7c6c5)cc4)c4cccc5c4ccc4ccccc45)cc3)c3c2oc2ccccc23)cc1] 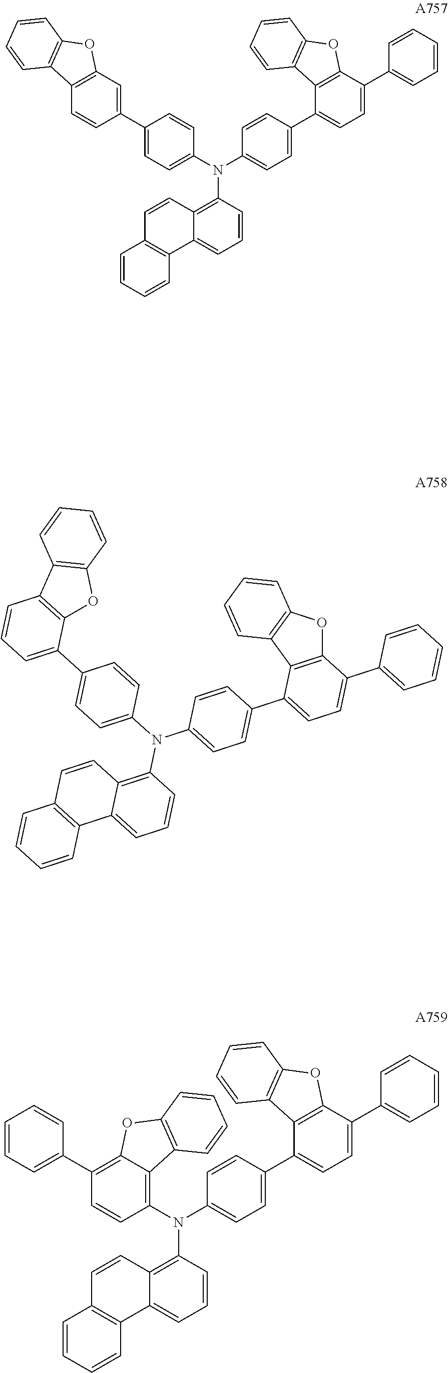 [SMILES: c1ccc(-c2ccc(-c3ccc(N(c4ccc(-c5ccc6c(c5)oc5ccccc56)cc4)c4cccc5c4ccc4ccccc45)cc3)c3c2oc2ccccc23)cc1 ; c1ccc(-c2ccc(-c3ccc(N(c4ccc(-c5cccc6c5oc5ccccc56)cc4)c4cccc5c4ccc4ccccc45)cc3)c3c2oc2ccccc23)cc1 ; c1ccc(-c2ccc(-c3ccc(N(c4cccc5c4ccc4ccccc45)c4ccc(-c5ccccc5)c5oc6ccccc6c45)cc3)c3c2oc2ccccc23)cc1] 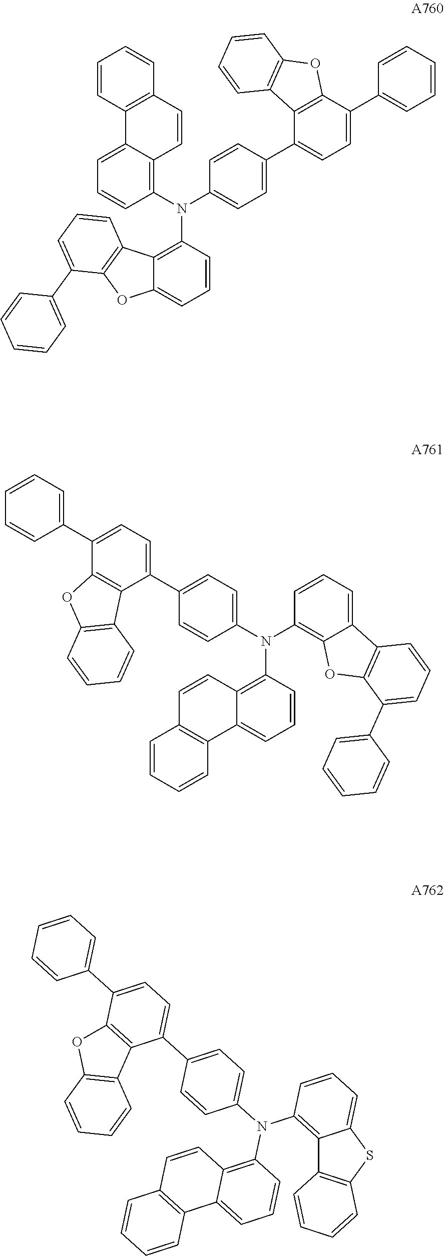 [SMILES: c1ccc(-c2cccc3c2oc2cccc(N(c4ccc(-c5ccc(-c6ccccc6)c6oc7ccccc7c56)cc4)c4cccc5c4ccc4ccccc45)c23)cc1 ; c1ccc(-c2cccc3c2oc2c(N(c4ccc(-c5ccc(-c6ccccc6)c6oc7ccccc7c56)cc4)c4cccc5c4ccc4ccccc45)cccc23)cc1 ; c1ccc(-c2ccc(-c3ccc(N(c4cccc5c4ccc4ccccc45)c4cccc5sc6ccccc6c45)cc3)c3c2oc2ccccc23)cc1] 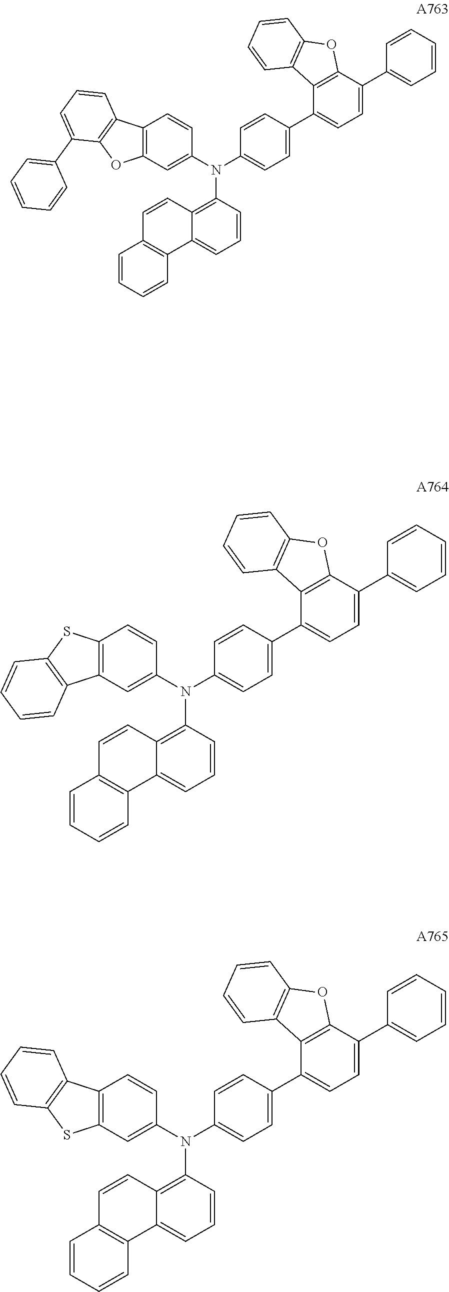 [SMILES: c1ccc(-c2cccc3c2oc2cc(N(c4ccc(-c5ccc(-c6ccccc6)c6oc7ccccc7c56)cc4)c4cccc5c4ccc4ccccc45)ccc23)cc1 ; c1ccc(-c2ccc(-c3ccc(N(c4ccc5sc6ccccc6c5c4)c4cccc5c4ccc4ccccc45)cc3)c3c2oc2ccccc23)cc1 ; c1ccc(-c2ccc(-c3ccc(N(c4ccc5c(c4)sc4ccccc45)c4cccc5c4ccc4ccccc45)cc3)c3c2oc2ccccc23)cc1] 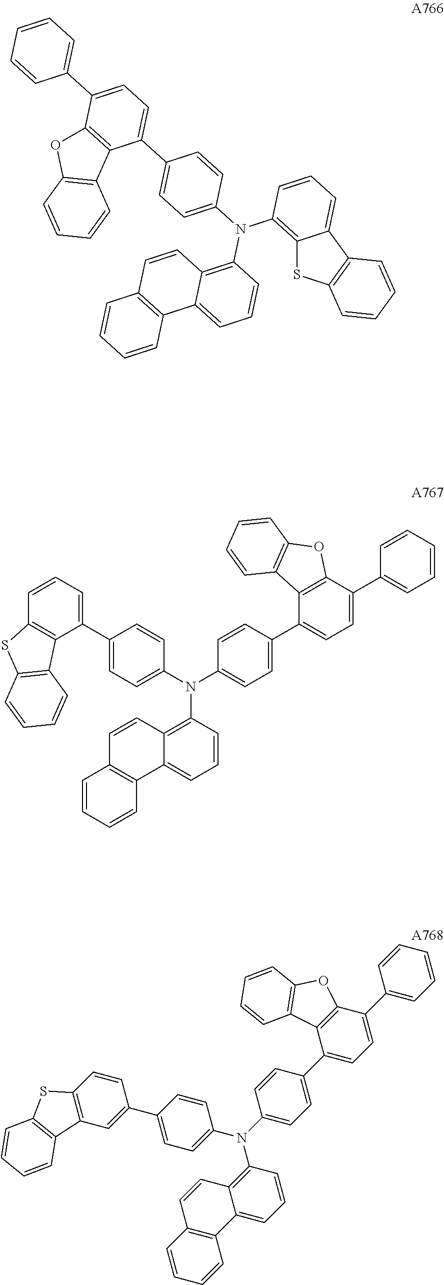 [SMILES: c1ccc(-c2ccc(-c3ccc(N(c4cccc5c4ccc4ccccc45)c4cccc5c4sc4ccccc45)cc3)c3c2oc2ccccc23)cc1 ; c1ccc(-c2ccc(-c3ccc(N(c4ccc(-c5cccc6sc7ccccc7c56)cc4)c4cccc5c4ccc4ccccc45)cc3)c3c2oc2ccccc23)cc1 ; c1ccc(-c2ccc(-c3ccc(N(c4ccc(-c5ccc6sc7ccccc7c6c5)cc4)c4cccc5c4ccc4ccccc45)cc3)c3c2oc2ccccc23)cc1] 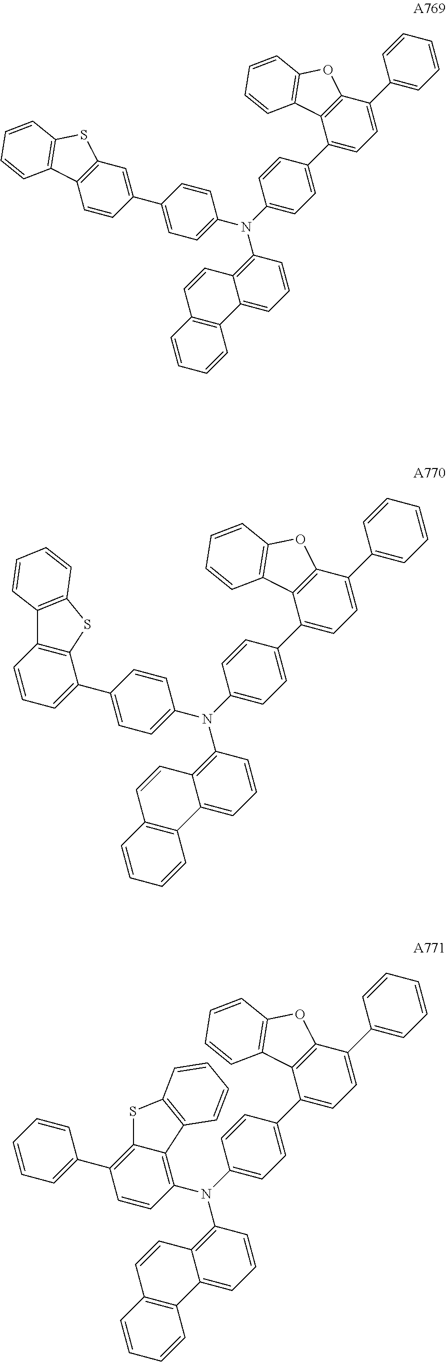 [SMILES: c1ccc(-c2ccc(-c3ccc(N(c4ccc(-c5ccc6c(c5)sc5ccccc56)cc4)c4cccc5c4ccc4ccccc45)cc3)c3c2oc2ccccc23)cc1 ; c1ccc(-c2ccc(-c3ccc(N(c4ccc(-c5cccc6c5sc5ccccc56)cc4)c4cccc5c4ccc4ccccc45)cc3)c3c2oc2ccccc23)cc1 ; c1ccc(-c2ccc(-c3ccc(N(c4cccc5c4ccc4ccccc45)c4ccc(-c5ccccc5)c5sc6ccccc6c45)cc3)c3c2oc2ccccc23)cc1] 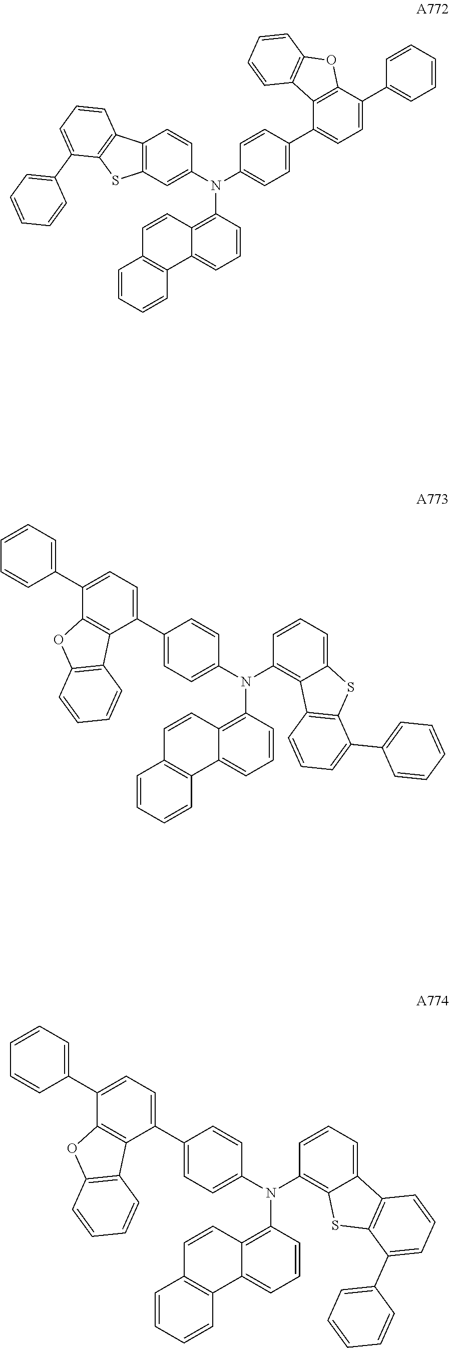 [SMILES: c1ccc(-c2ccc(-c3ccc(N(c4ccc5c(c4)sc4c(-c6ccccc6)cccc45)c4cccc5c4ccc4ccccc45)cc3)c3c2oc2ccccc23)cc1 ; c1ccc(-c2ccc(-c3ccc(N(c4cccc5c4ccc4ccccc45)c4cccc5sc6c(-c7ccccc7)cccc6c45)cc3)c3c2oc2ccccc23)cc1 ; c1ccc(-c2ccc(-c3ccc(N(c4cccc5c4ccc4ccccc45)c4cccc5c4sc4c(-c6ccccc6)cccc45)cc3)c3c2oc2ccccc23)cc1] 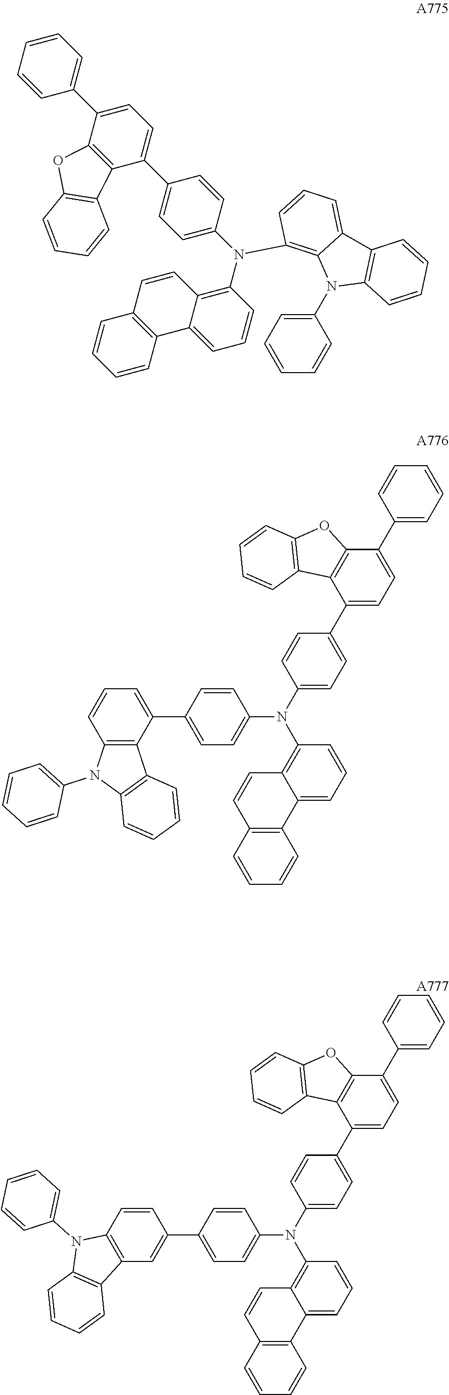 [SMILES: c1ccc(-c2ccc(-c3ccc(N(c4cccc5c4ccc4ccccc45)c4cccc5c6ccccc6n(-c6ccccc6)c45)cc3)c3c2oc2ccccc23)cc1 ; c1ccc(-c2ccc(-c3ccc(N(c4ccc(-c5cccc6c5c5ccccc5n6-c5ccccc5)cc4)c4cccc5c4ccc4ccccc45)cc3)c3c2oc2ccccc23)cc1 ; c1ccc(-c2ccc(-c3ccc(N(c4ccc(-c5ccc6c(c5)c5ccccc5n6-c5ccccc5)cc4)c4cccc5c4ccc4ccccc45)cc3)c3c2oc2ccccc23)cc1] 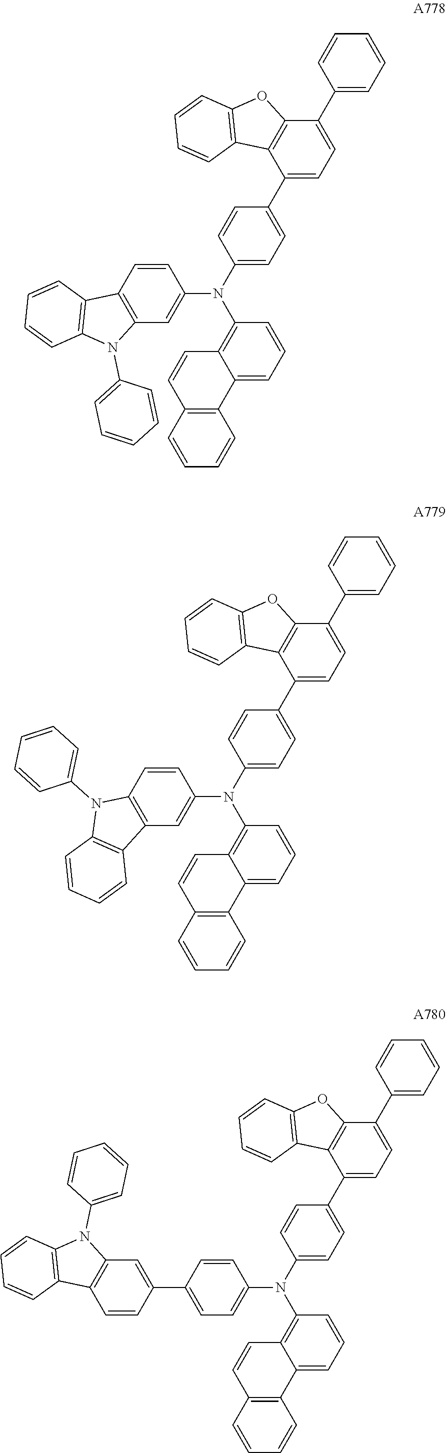 [SMILES: c1ccc(-c2ccc(-c3ccc(N(c4ccc5c6ccccc6n(-c6ccccc6)c5c4)c4cccc5c4ccc4ccccc45)cc3)c3c2oc2ccccc23)cc1 ; c1ccc(-c2ccc(-c3ccc(N(c4ccc5c(c4)c4ccccc4n5-c4ccccc4)c4cccc5c4ccc4ccccc45)cc3)c3c2oc2ccccc23)cc1 ; c1ccc(-c2ccc(-c3ccc(N(c4ccc(-c5ccc6c7ccccc7n(-c7ccccc7)c6c5)cc4)c4cccc5c4ccc4ccccc45)cc3)c3c2oc2ccccc23)cc1] 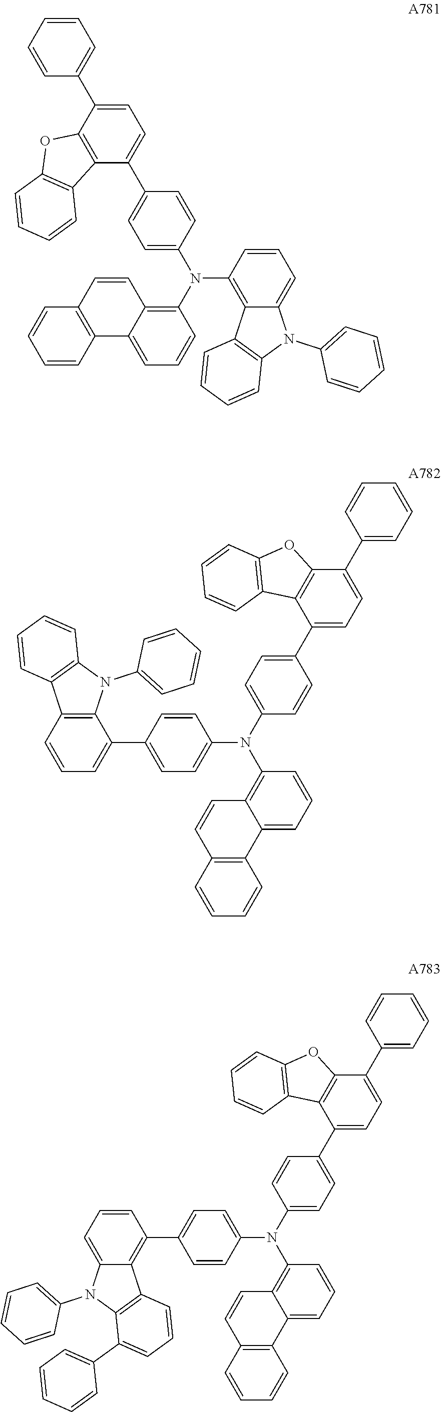 [SMILES: c1ccc(-c2ccc(-c3ccc(N(c4cccc5c4ccc4ccccc45)c4cccc5c4c4ccccc4n5-c4ccccc4)cc3)c3c2oc2ccccc23)cc1 ; c1ccc(-c2ccc(-c3ccc(N(c4ccc(-c5cccc6c7ccccc7n(-c7ccccc7)c56)cc4)c4cccc5c4ccc4ccccc45)cc3)c3c2oc2ccccc23)cc1 ; c1ccc(-c2ccc(-c3ccc(N(c4ccc(-c5cccc6c5c5cccc(-c7ccccc7)c5n6-c5ccccc5)cc4)c4cccc5c4ccc4ccccc45)cc3)c3c2oc2ccccc23)cc1] 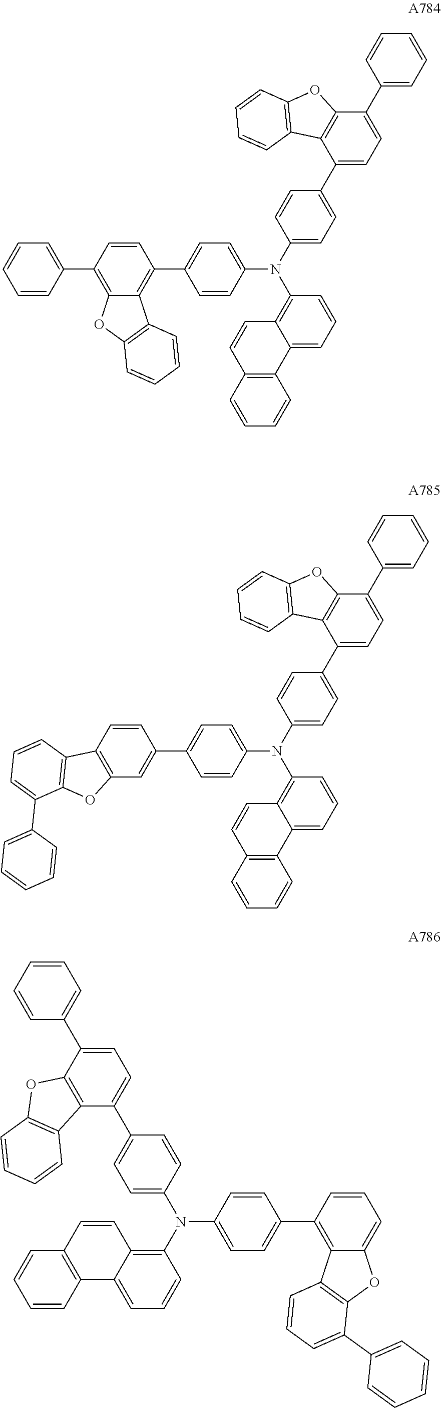 [SMILES: c1ccc(-c2ccc(-c3ccc(N(c4ccc(-c5ccc(-c6ccccc6)c6oc7ccccc7c56)cc4)c4cccc5c4ccc4ccccc45)cc3)c3c2oc2ccccc23)cc1 ; c1ccc(-c2cccc3c2oc2cc(-c4ccc(N(c5ccc(-c6ccc(-c7ccccc7)c7oc8ccccc8c67)cc5)c5cccc6c5ccc5ccccc56)cc4)ccc23)cc1 ; c1ccc(-c2cccc3c2oc2cccc(-c4ccc(N(c5ccc(-c6ccc(-c7ccccc7)c7oc8ccccc8c67)cc5)c5cccc6c5ccc5ccccc56)cc4)c23)cc1] 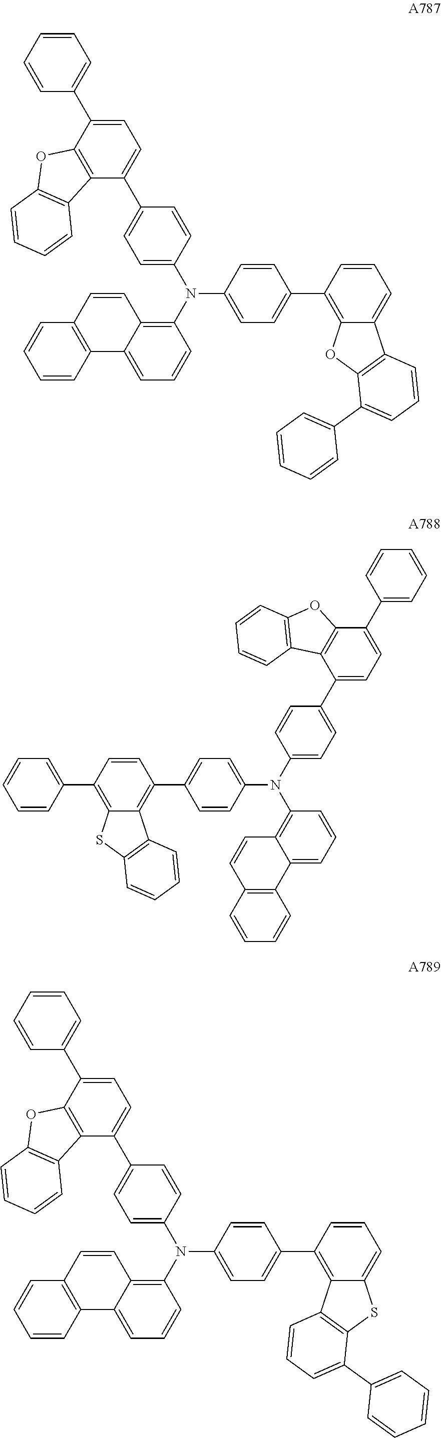 [SMILES: c1ccc(-c2cccc3c2oc2c(-c4ccc(N(c5ccc(-c6ccc(-c7ccccc7)c7oc8ccccc8c67)cc5)c5cccc6c5ccc5ccccc56)cc4)cccc23)cc1 ; c1ccc(-c2ccc(-c3ccc(N(c4ccc(-c5ccc(-c6ccccc6)c6sc7ccccc7c56)cc4)c4cccc5c4ccc4ccccc45)cc3)c3c2oc2ccccc23)cc1 ; c1ccc(-c2ccc(-c3ccc(N(c4ccc(-c5cccc6sc7c(-c8ccccc8)cccc7c56)cc4)c4cccc5c4ccc4ccccc45)cc3)c3c2oc2ccccc23)cc1] 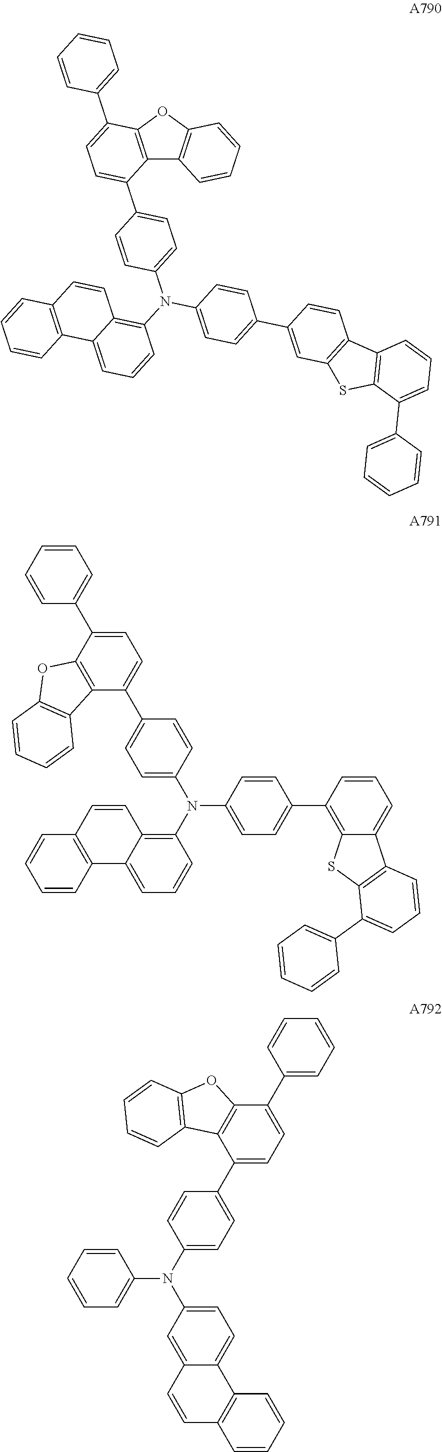 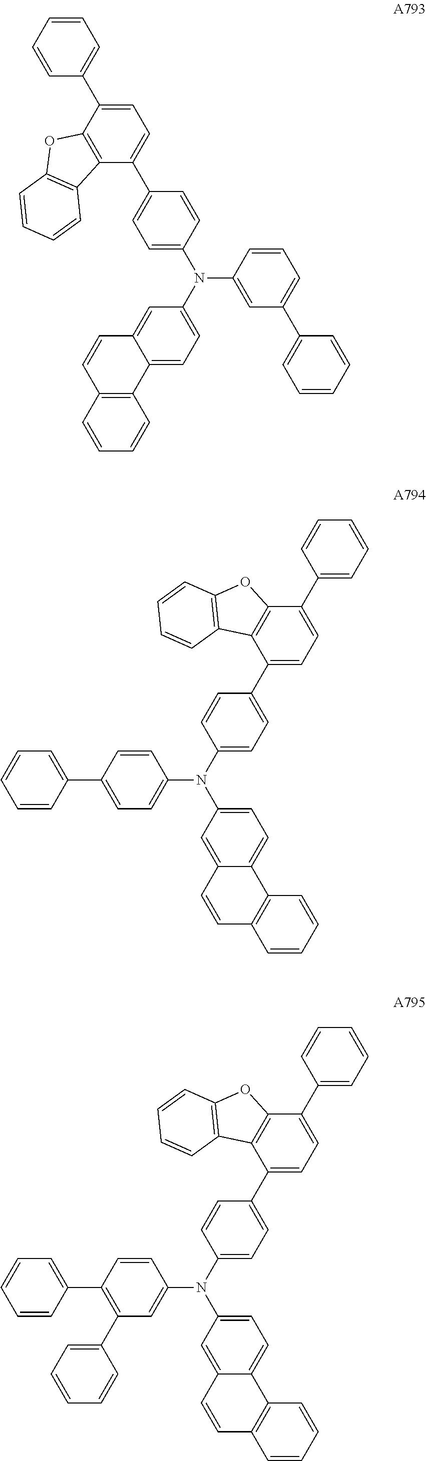 [SMILES: c1ccc(-c2cccc(N(c3ccc(-c4ccc(-c5ccccc5)c5oc6ccccc6c45)cc3)c3ccc4c(ccc5ccccc54)c3)c2)cc1 ; c1ccc(-c2ccc(N(c3ccc(-c4ccc(-c5ccccc5)c5oc6ccccc6c45)cc3)c3ccc4c(ccc5ccccc54)c3)cc2)cc1 ; c1ccc(-c2ccc(N(c3ccc(-c4ccc(-c5ccccc5)c5oc6ccccc6c45)cc3)c3ccc4c(ccc5ccccc54)c3)cc2-c2ccccc2)cc1] 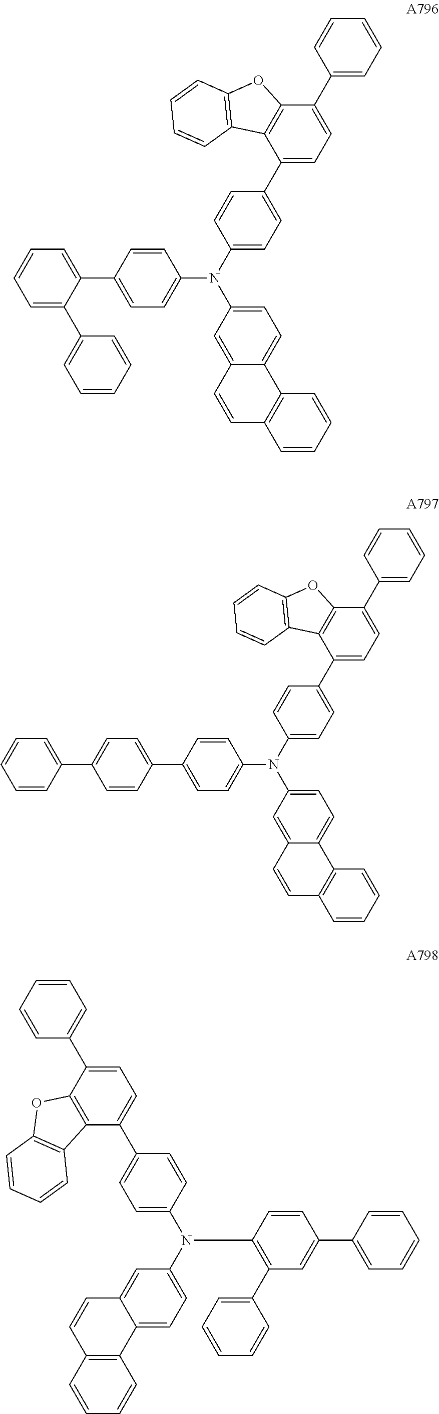 [SMILES: c1ccc(-c2ccccc2-c2ccc(N(c3ccc(-c4ccc(-c5ccccc5)c5oc6ccccc6c45)cc3)c3ccc4c(ccc5ccccc54)c3)cc2)cc1 ; c1ccc(-c2ccc(-c3ccc(N(c4ccc(-c5ccc(-c6ccccc6)c6oc7ccccc7c56)cc4)c4ccc5c(ccc6ccccc65)c4)cc3)cc2)cc1 ; c1ccc(-c2ccc(N(c3ccc(-c4ccc(-c5ccccc5)c5oc6ccccc6c45)cc3)c3ccc4c(ccc5ccccc54)c3)c(-c3ccccc3)c2)cc1] 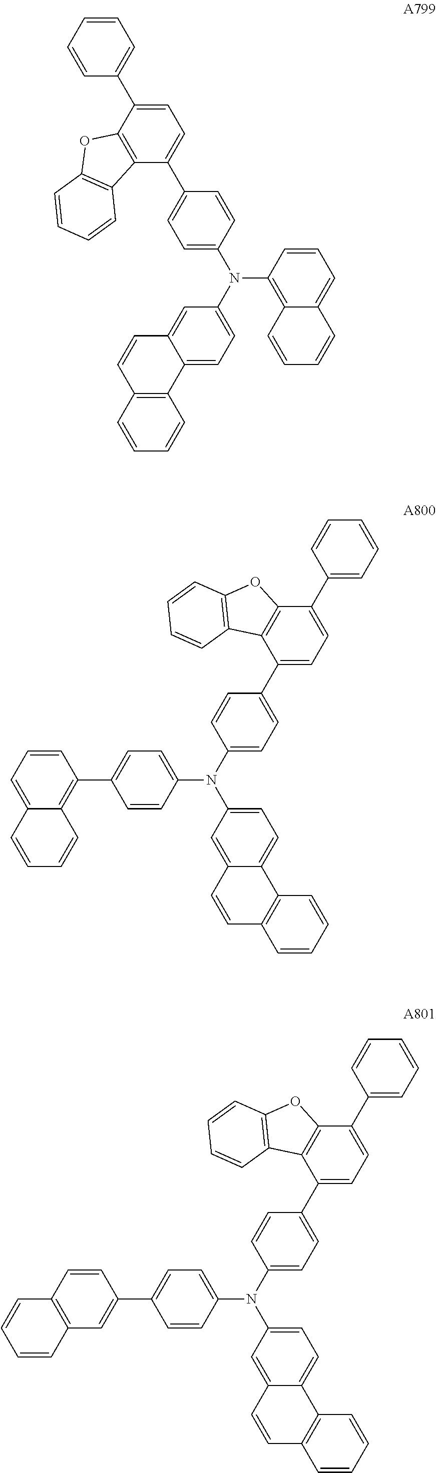 [SMILES: c1ccc(-c2ccc(-c3ccc(N(c4ccc5c(ccc6ccccc65)c4)c4cccc5ccccc45)cc3)c3c2oc2ccccc23)cc1 ; c1ccc(-c2ccc(-c3ccc(N(c4ccc(-c5cccc6ccccc56)cc4)c4ccc5c(ccc6ccccc65)c4)cc3)c3c2oc2ccccc23)cc1 ; c1ccc(-c2ccc(-c3ccc(N(c4ccc(-c5ccc6ccccc6c5)cc4)c4ccc5c(ccc6ccccc65)c4)cc3)c3c2oc2ccccc23)cc1] 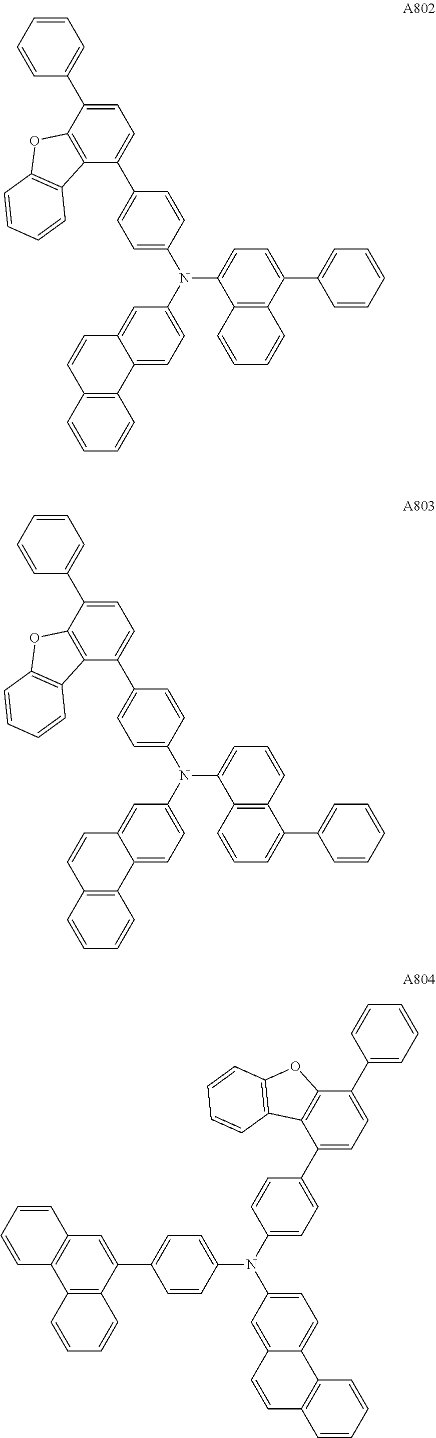 [SMILES: c1ccc(-c2ccc(N(c3ccc(-c4ccc(-c5ccccc5)c5oc6ccccc6c45)cc3)c3ccc4c(ccc5ccccc54)c3)c3ccccc23)cc1 ; c1ccc(-c2cccc3c(N(c4ccc(-c5ccc(-c6ccccc6)c6oc7ccccc7c56)cc4)c4ccc5c(ccc6ccccc65)c4)cccc23)cc1 ; c1ccc(-c2ccc(-c3ccc(N(c4ccc(-c5cc6ccccc6c6ccccc56)cc4)c4ccc5c(ccc6ccccc65)c4)cc3)c3c2oc2ccccc23)cc1] 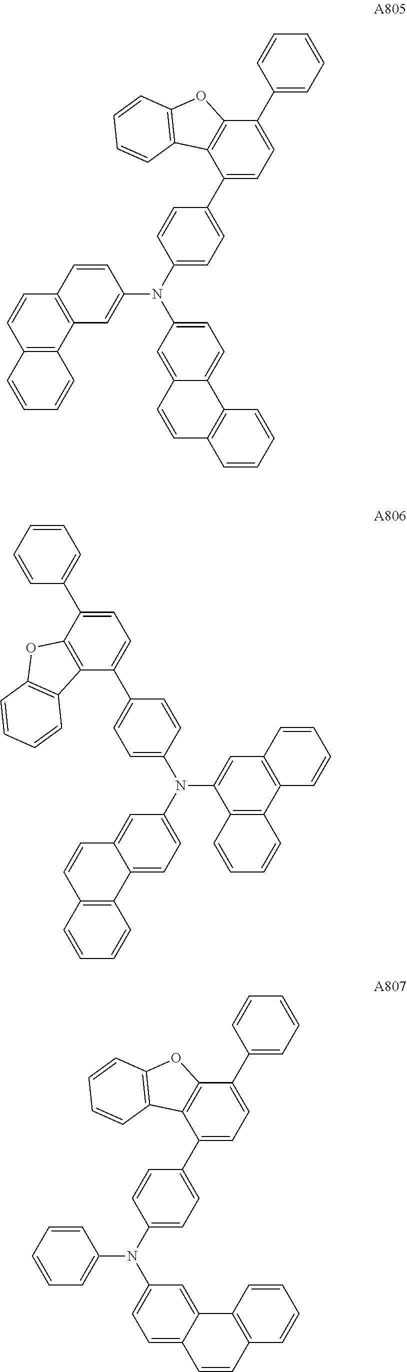 [SMILES: c1ccc(-c2ccc(-c3ccc(N(c4ccc5c(ccc6ccccc65)c4)c4ccc5ccc6ccccc6c5c4)cc3)c3c2oc2ccccc23)cc1 ; c1ccc(-c2ccc(-c3ccc(N(c4ccc5c(ccc6ccccc65)c4)c4cc5ccccc5c5ccccc45)cc3)c3c2oc2ccccc23)cc1 ; c1ccc(-c2ccc(-c3ccc(N(c4ccccc4)c4ccc5ccc6ccccc6c5c4)cc3)c3c2oc2ccccc23)cc1] 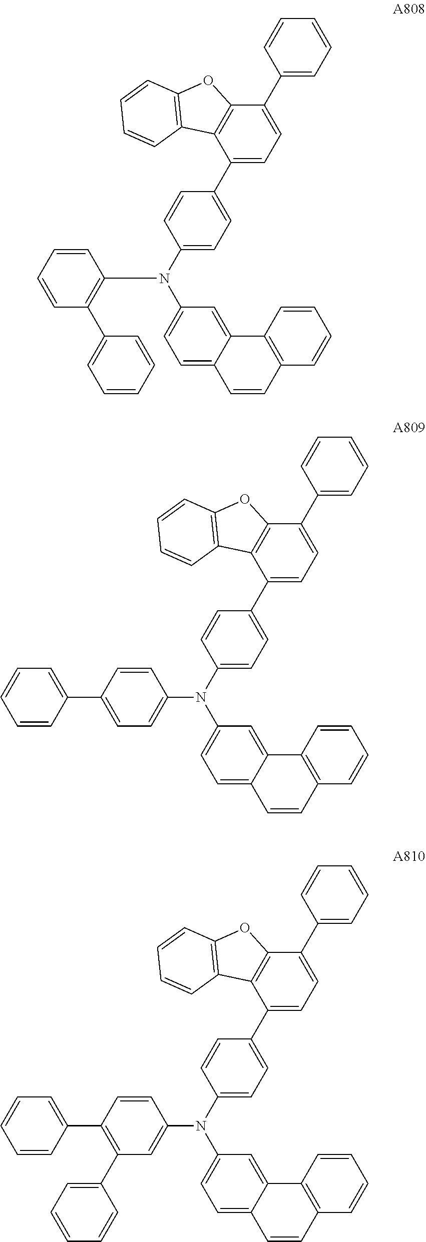 [SMILES: c1ccc(-c2ccccc2N(c2ccc(-c3ccc(-c4ccccc4)c4oc5ccccc5c34)cc2)c2ccc3ccc4ccccc4c3c2)cc1 ; c1ccc(-c2ccc(N(c3ccc(-c4ccc(-c5ccccc5)c5oc6ccccc6c45)cc3)c3ccc4ccc5ccccc5c4c3)cc2)cc1 ; c1ccc(-c2ccc(N(c3ccc(-c4ccc(-c5ccccc5)c5oc6ccccc6c45)cc3)c3ccc4ccc5ccccc5c4c3)cc2-c2ccccc2)cc1] 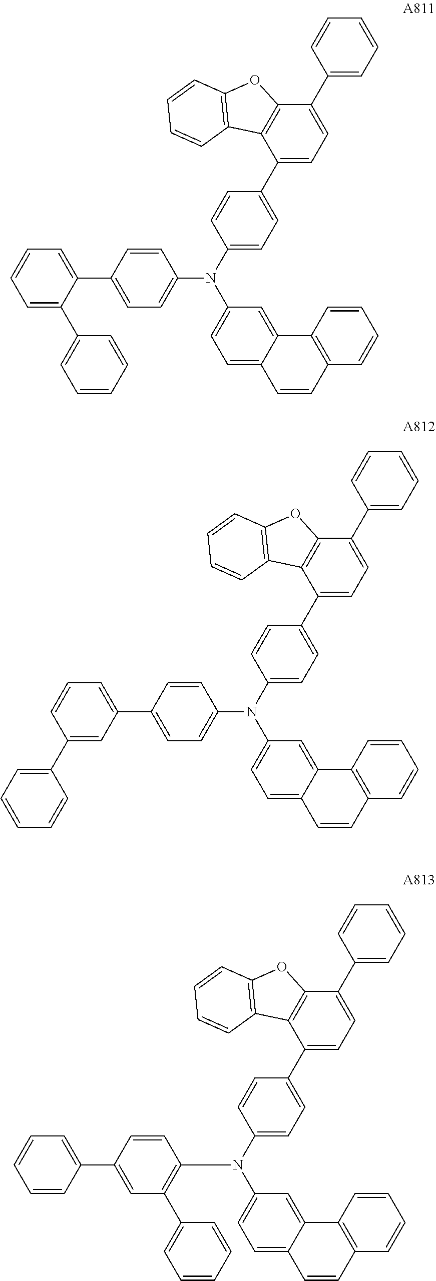 [SMILES: c1ccc(-c2ccccc2-c2ccc(N(c3ccc(-c4ccc(-c5ccccc5)c5oc6ccccc6c45)cc3)c3ccc4ccc5ccccc5c4c3)cc2)cc1 ; c1ccc(-c2cccc(-c3ccc(N(c4ccc(-c5ccc(-c6ccccc6)c6oc7ccccc7c56)cc4)c4ccc5ccc6ccccc6c5c4)cc3)c2)cc1 ; c1ccc(-c2ccc(N(c3ccc(-c4ccc(-c5ccccc5)c5oc6ccccc6c45)cc3)c3ccc4ccc5ccccc5c4c3)c(-c3ccccc3)c2)cc1] 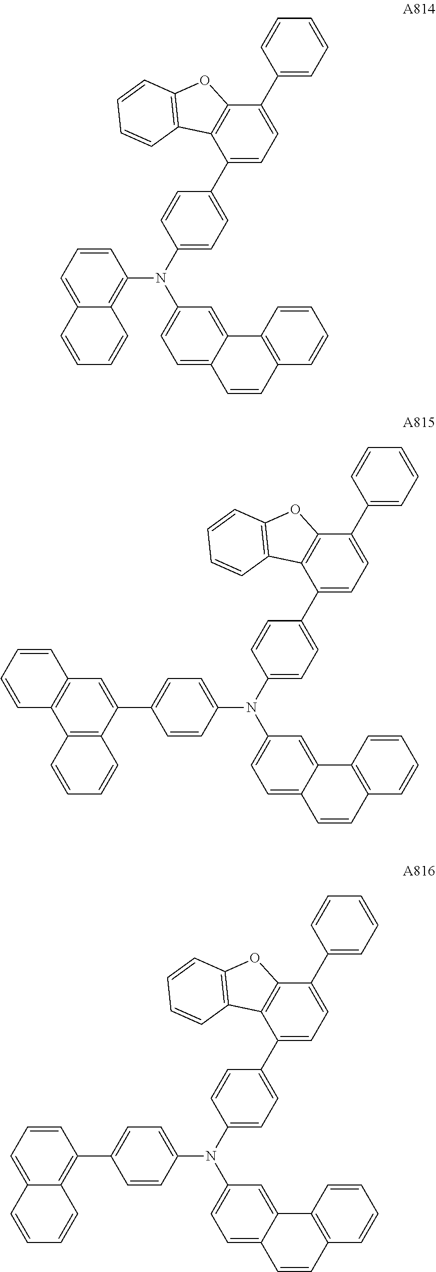 [SMILES: c1ccc(-c2ccc(-c3ccc(N(c4ccc5ccc6ccccc6c5c4)c4cccc5ccccc45)cc3)c3c2oc2ccccc23)cc1 ; c1ccc(-c2ccc(-c3ccc(N(c4ccc(-c5cc6ccccc6c6ccccc56)cc4)c4ccc5ccc6ccccc6c5c4)cc3)c3c2oc2ccccc23)cc1 ; c1ccc(-c2ccc(-c3ccc(N(c4ccc(-c5cccc6ccccc56)cc4)c4ccc5ccc6ccccc6c5c4)cc3)c3c2oc2ccccc23)cc1] 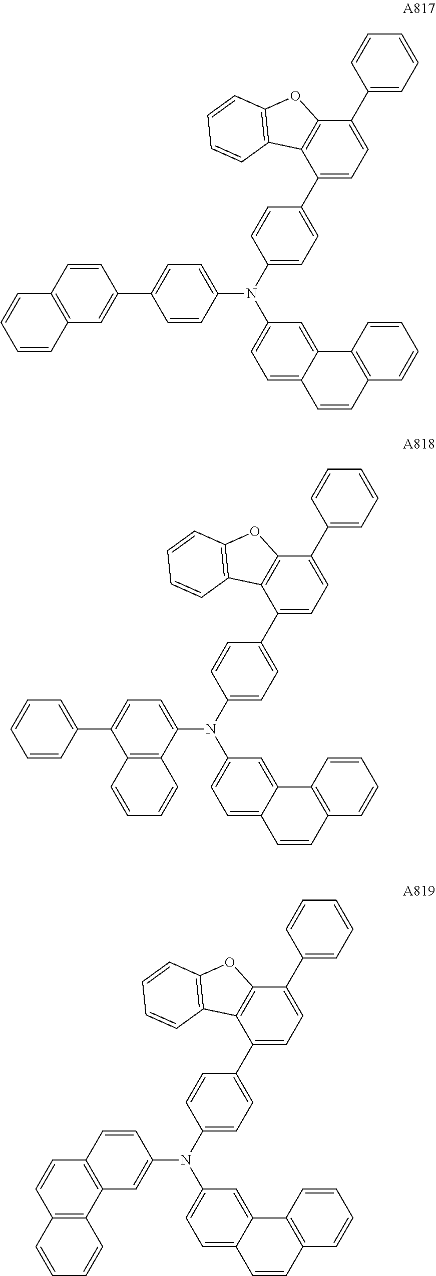 [SMILES: c1ccc(-c2ccc(-c3ccc(N(c4ccc(-c5ccc6ccccc6c5)cc4)c4ccc5ccc6ccccc6c5c4)cc3)c3c2oc2ccccc23)cc1 ; c1ccc(-c2ccc(N(c3ccc(-c4ccc(-c5ccccc5)c5oc6ccccc6c45)cc3)c3ccc4ccc5ccccc5c4c3)c3ccccc23)cc1 ; c1ccc(-c2ccc(-c3ccc(N(c4ccc5ccc6ccccc6c5c4)c4ccc5ccc6ccccc6c5c4)cc3)c3c2oc2ccccc23)cc1] 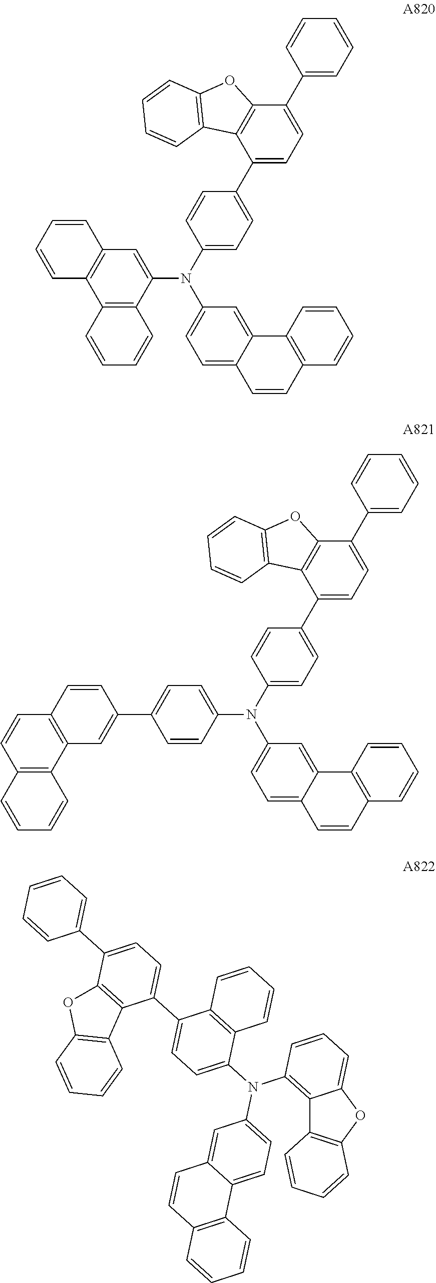 [SMILES: c1ccc(-c2ccc(-c3ccc(N(c4ccc5ccc6ccccc6c5c4)c4cc5ccccc5c5ccccc45)cc3)c3c2oc2ccccc23)cc1 ; c1ccc(-c2ccc(-c3ccc(N(c4ccc(-c5ccc6ccc7ccccc7c6c5)cc4)c4ccc5ccc6ccccc6c5c4)cc3)c3c2oc2ccccc23)cc1 ; c1ccc(-c2ccc(-c3ccc(N(c4ccc5c(ccc6ccccc65)c4)c4cccc5oc6ccccc6c45)c4ccccc34)c3c2oc2ccccc23)cc1] 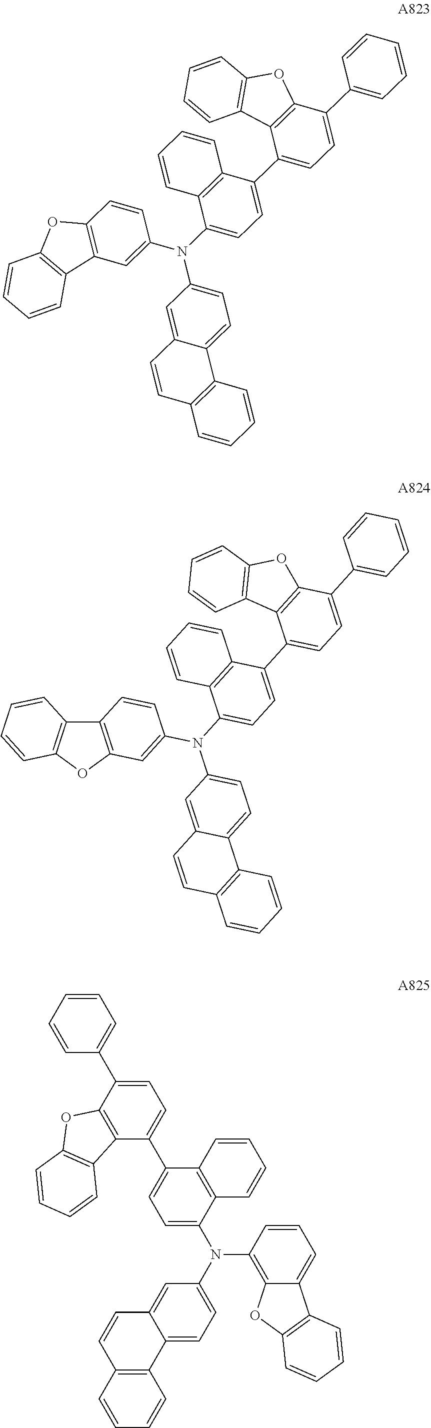 [SMILES: c1ccc(-c2ccc(-c3ccc(N(c4ccc5c(ccc6ccccc65)c4)c4ccc5oc6ccccc6c5c4)c4ccccc34)c3c2oc2ccccc23)cc1 ; c1ccc(-c2ccc(-c3ccc(N(c4ccc5c(ccc6ccccc65)c4)c4ccc5c(c4)oc4ccccc45)c4ccccc34)c3c2oc2ccccc23)cc1 ; c1ccc(-c2ccc(-c3ccc(N(c4ccc5c(ccc6ccccc65)c4)c4cccc5c4oc4ccccc45)c4ccccc34)c3c2oc2ccccc23)cc1] 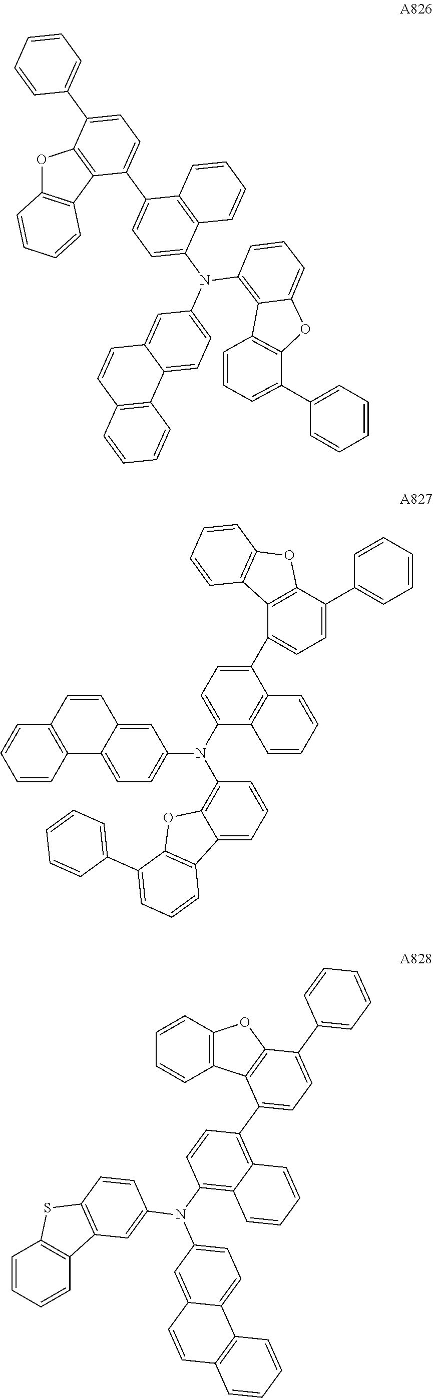 [SMILES: c1ccc(-c2cccc3c2oc2cccc(N(c4ccc5c(ccc6ccccc65)c4)c4ccc(-c5ccc(-c6ccccc6)c6oc7ccccc7c56)c5ccccc45)c23)cc1 ; c1ccc(-c2cccc3c2oc2c(N(c4ccc5c(ccc6ccccc65)c4)c4ccc(-c5ccc(-c6ccccc6)c6oc7ccccc7c56)c5ccccc45)cccc23)cc1 ; c1ccc(-c2ccc(-c3ccc(N(c4ccc5c(ccc6ccccc65)c4)c4ccc5sc6ccccc6c5c4)c4ccccc34)c3c2oc2ccccc23)cc1] 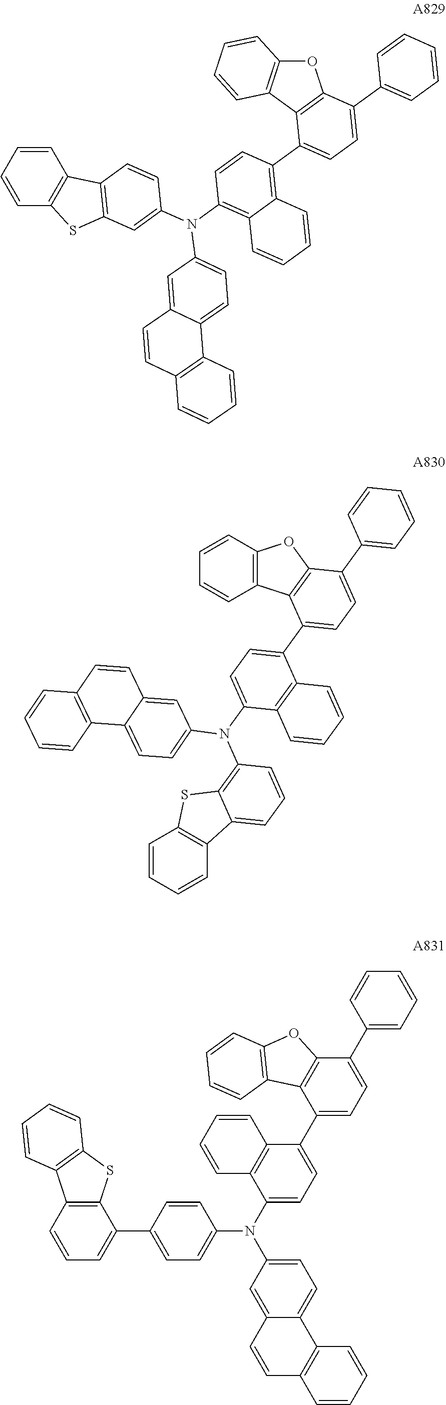 [SMILES: c1ccc(-c2ccc(-c3ccc(N(c4ccc5c(ccc6ccccc65)c4)c4ccc5c(c4)sc4ccccc45)c4ccccc34)c3c2oc2ccccc23)cc1 ; c1ccc(-c2ccc(-c3ccc(N(c4ccc5c(ccc6ccccc65)c4)c4cccc5c4sc4ccccc45)c4ccccc34)c3c2oc2ccccc23)cc1 ; c1ccc(-c2ccc(-c3ccc(N(c4ccc(-c5cccc6c5sc5ccccc56)cc4)c4ccc5c(ccc6ccccc65)c4)c4ccccc34)c3c2oc2ccccc23)cc1] 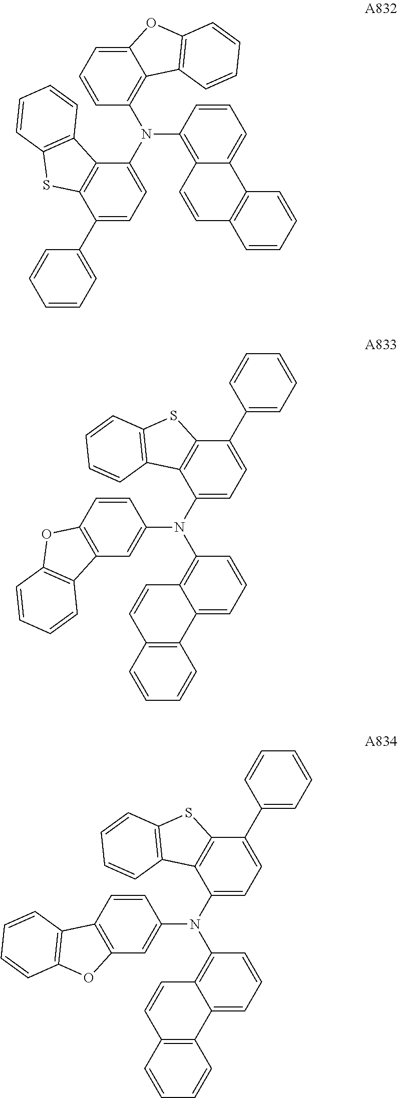 [SMILES: c1ccc(-c2ccc(N(c3cccc4c3ccc3ccccc34)c3cccc4oc5ccccc5c34)c3c2sc2ccccc23)cc1 ; c1ccc(-c2ccc(N(c3ccc4oc5ccccc5c4c3)c3cccc4c3ccc3ccccc34)c3c2sc2ccccc23)cc1 ; c1ccc(-c2ccc(N(c3ccc4c(c3)oc3ccccc34)c3cccc4c3ccc3ccccc34)c3c2sc2ccccc23)cc1] 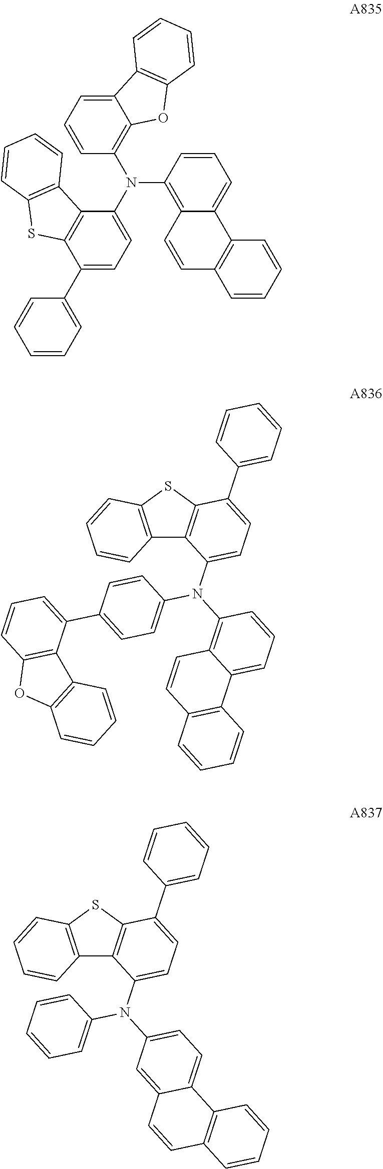 [SMILES: c1ccc(-c2ccc(N(c3cccc4c3ccc3ccccc34)c3cccc4c3oc3ccccc34)c3c2sc2ccccc23)cc1 ; c1ccc(-c2ccc(N(c3ccc(-c4cccc5oc6ccccc6c45)cc3)c3cccc4c3ccc3ccccc34)c3c2sc2ccccc23)cc1 ; c1ccc(-c2ccc(N(c3ccccc3)c3ccc4c(ccc5ccccc54)c3)c3c2sc2ccccc23)cc1] 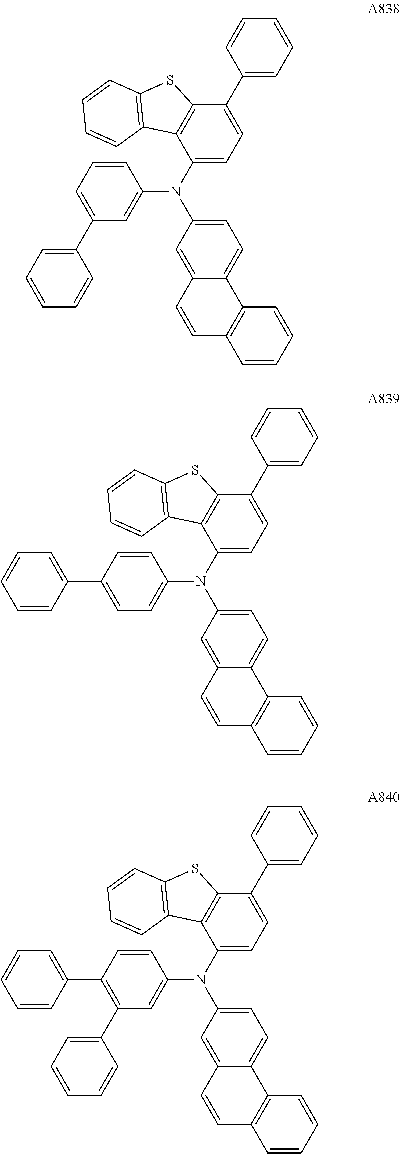 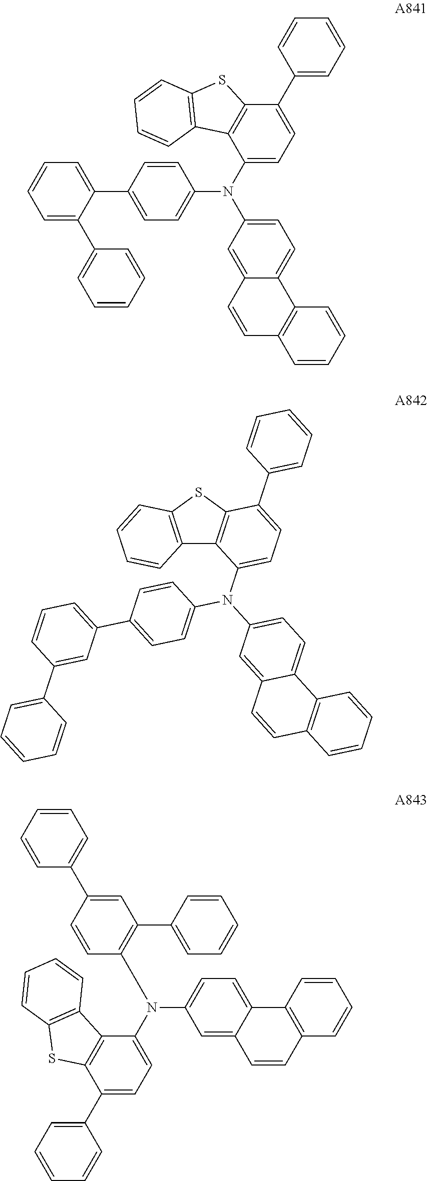 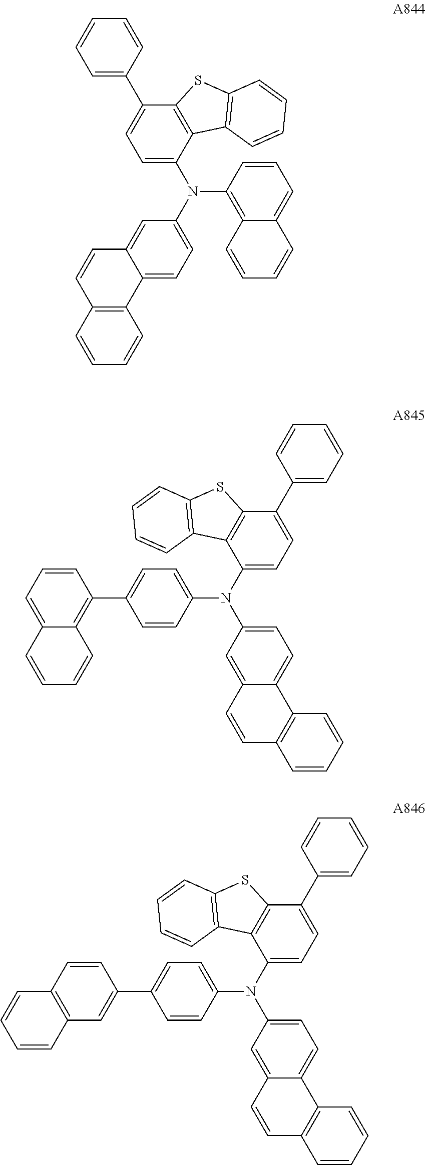 [SMILES: c1ccc(-c2ccc(N(c3ccc4c(ccc5ccccc54)c3)c3cccc4ccccc34)c3c2sc2ccccc23)cc1 ; c1ccc(-c2ccc(N(c3ccc(-c4cccc5ccccc45)cc3)c3ccc4c(ccc5ccccc54)c3)c3c2sc2ccccc23)cc1 ; c1ccc(-c2ccc(N(c3ccc(-c4ccc5ccccc5c4)cc3)c3ccc4c(ccc5ccccc54)c3)c3c2sc2ccccc23)cc1] 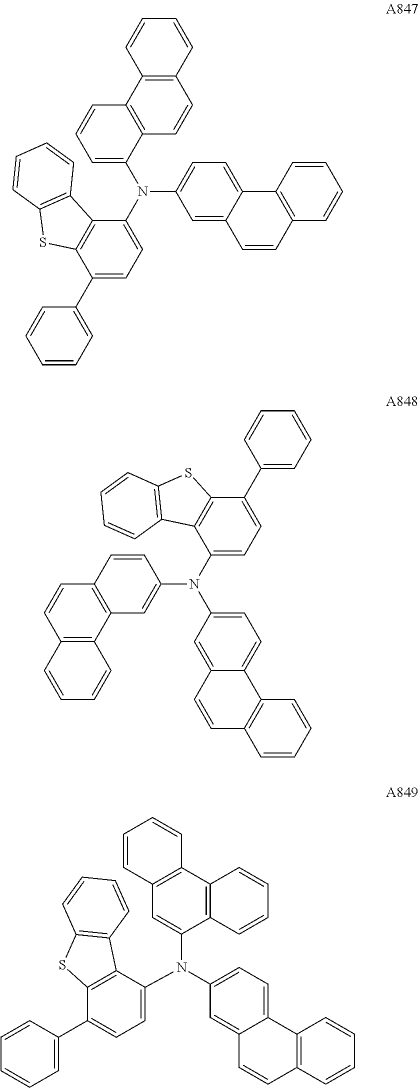 [SMILES: c1ccc(-c2ccc(N(c3ccc4c(ccc5ccccc54)c3)c3cccc4c3ccc3ccccc34)c3c2sc2ccccc23)cc1 ; c1ccc(-c2ccc(N(c3ccc4c(ccc5ccccc54)c3)c3ccc4ccc5ccccc5c4c3)c3c2sc2ccccc23)cc1 ; c1ccc(-c2ccc(N(c3ccc4c(ccc5ccccc54)c3)c3cc4ccccc4c4ccccc34)c3c2sc2ccccc23)cc1] 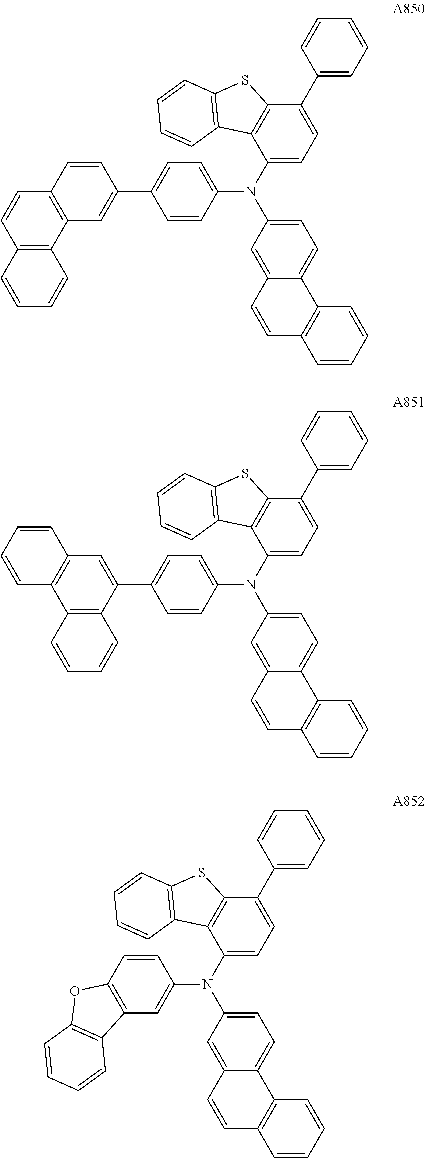 [SMILES: c1ccc(-c2ccc(N(c3ccc(-c4ccc5ccc6ccccc6c5c4)cc3)c3ccc4c(ccc5ccccc54)c3)c3c2sc2ccccc23)cc1 ; c1ccc(-c2ccc(N(c3ccc(-c4cc5ccccc5c5ccccc45)cc3)c3ccc4c(ccc5ccccc54)c3)c3c2sc2ccccc23)cc1 ; c1ccc(-c2ccc(N(c3ccc4c(ccc5ccccc54)c3)c3ccc4oc5ccccc5c4c3)c3c2sc2ccccc23)cc1] 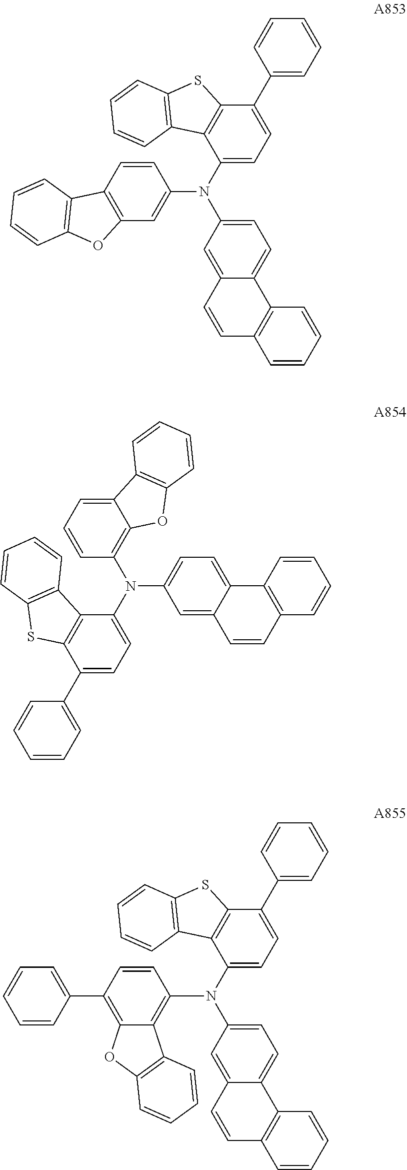 [SMILES: c1ccc(-c2ccc(N(c3ccc4c(ccc5ccccc54)c3)c3ccc4c(c3)oc3ccccc34)c3c2sc2ccccc23)cc1 ; c1ccc(-c2ccc(N(c3ccc4c(ccc5ccccc54)c3)c3cccc4c3oc3ccccc34)c3c2sc2ccccc23)cc1 ; c1ccc(-c2ccc(N(c3ccc4c(ccc5ccccc54)c3)c3ccc(-c4ccccc4)c4sc5ccccc5c34)c3c2oc2ccccc23)cc1] 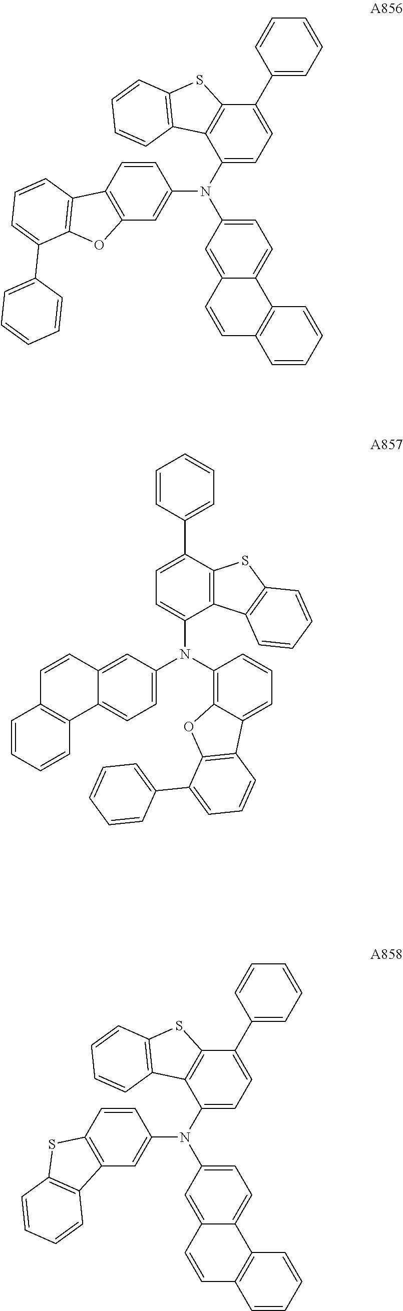 [SMILES: c1ccc(-c2cccc3c2oc2cc(N(c4ccc5c(ccc6ccccc65)c4)c4ccc(-c5ccccc5)c5sc6ccccc6c45)ccc23)cc1 ; c1ccc(-c2cccc3c2oc2c(N(c4ccc5c(ccc6ccccc65)c4)c4ccc(-c5ccccc5)c5sc6ccccc6c45)cccc23)cc1 ; c1ccc(-c2ccc(N(c3ccc4c(ccc5ccccc54)c3)c3ccc4sc5ccccc5c4c3)c3c2sc2ccccc23)cc1] 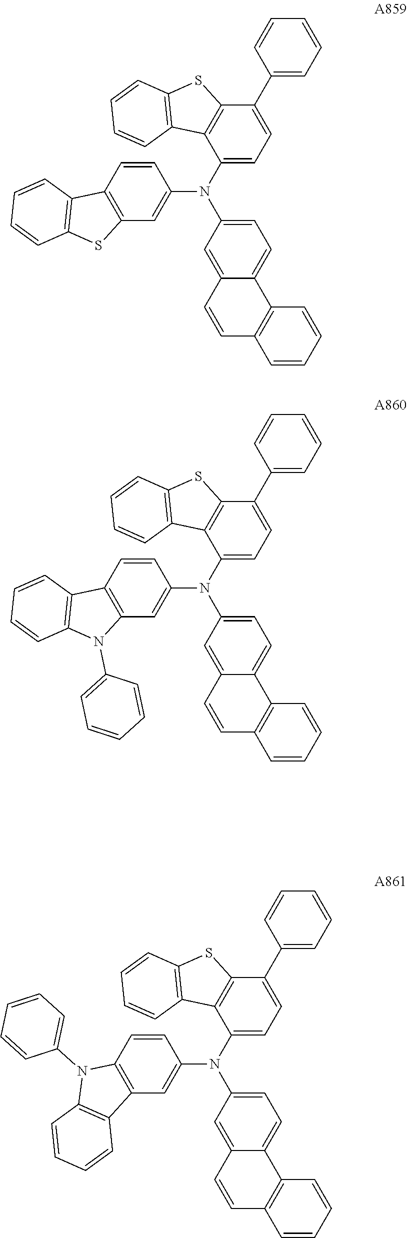 [SMILES: c1ccc(-c2ccc(N(c3ccc4c(ccc5ccccc54)c3)c3ccc4c(c3)sc3ccccc34)c3c2sc2ccccc23)cc1 ; c1ccc(-c2ccc(N(c3ccc4c(ccc5ccccc54)c3)c3ccc4c5ccccc5n(-c5ccccc5)c4c3)c3c2sc2ccccc23)cc1 ; c1ccc(-c2ccc(N(c3ccc4c(ccc5ccccc54)c3)c3ccc4c(c3)c3ccccc3n4-c3ccccc3)c3c2sc2ccccc23)cc1] 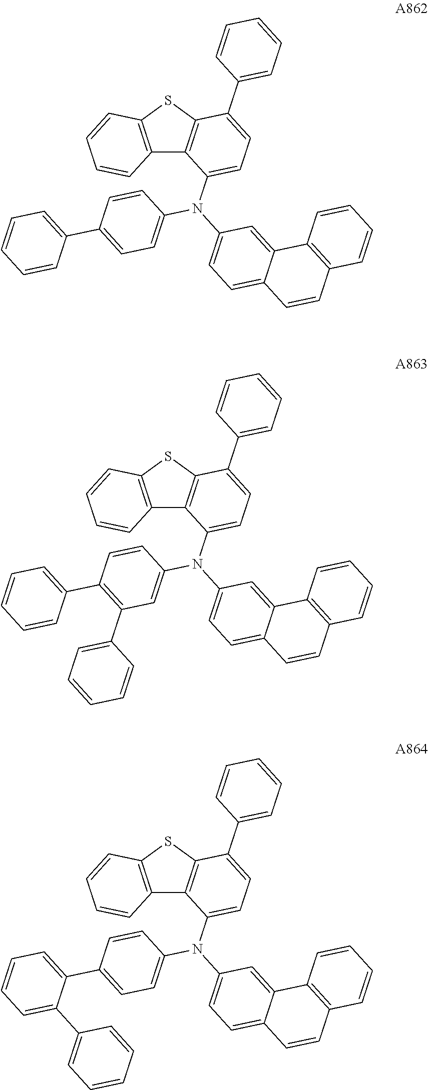 [SMILES: c1ccc(-c2ccc(N(c3ccc4ccc5ccccc5c4c3)c3ccc(-c4ccccc4)c4sc5ccccc5c34)cc2)cc1 ; c1ccc(-c2ccc(N(c3ccc4ccc5ccccc5c4c3)c3ccc(-c4ccccc4)c4sc5ccccc5c34)cc2-c2ccccc2)cc1 ; c1ccc(-c2ccccc2-c2ccc(N(c3ccc4ccc5ccccc5c4c3)c3ccc(-c4ccccc4)c4sc5ccccc5c34)cc2)cc1] 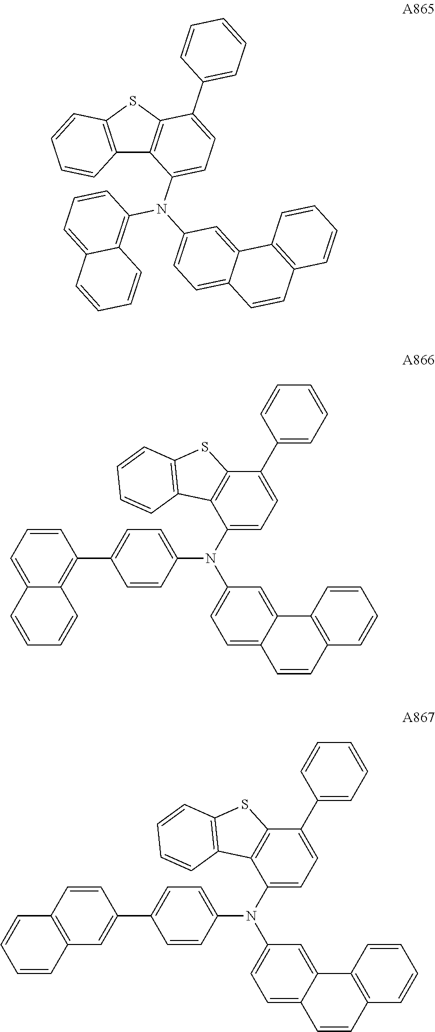 [SMILES: c1ccc(-c2ccc(N(c3ccc4ccc5ccccc5c4c3)c3cccc4ccccc34)c3c2sc2ccccc23)cc1 ; c1ccc(-c2ccc(N(c3ccc(-c4cccc5ccccc45)cc3)c3ccc4ccc5ccccc5c4c3)c3c2sc2ccccc23)cc1 ; c1ccc(-c2ccc(N(c3ccc(-c4ccc5ccccc5c4)cc3)c3ccc4ccc5ccccc5c4c3)c3c2sc2ccccc23)cc1] 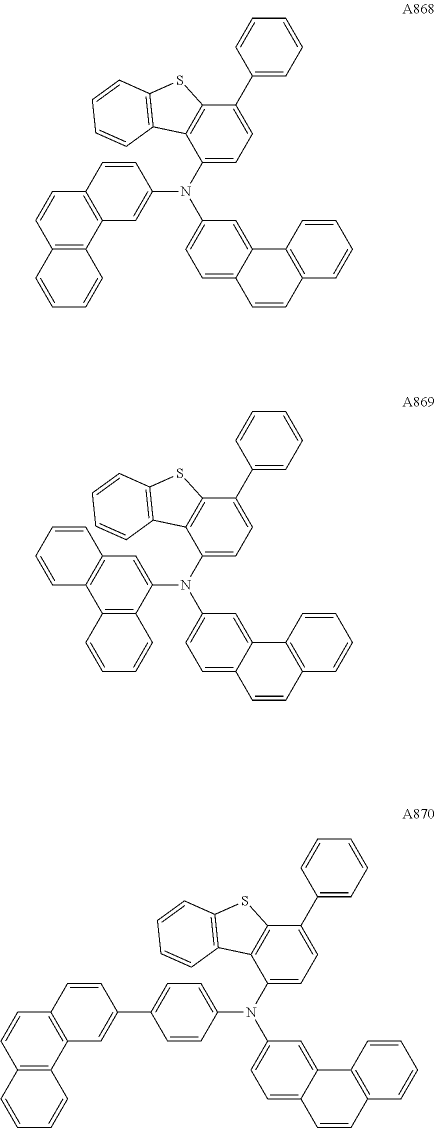 [SMILES: c1ccc(-c2ccc(N(c3ccc4ccc5ccccc5c4c3)c3ccc4ccc5ccccc5c4c3)c3c2sc2ccccc23)cc1 ; c1ccc(-c2ccc(N(c3ccc4ccc5ccccc5c4c3)c3cc4ccccc4c4ccccc34)c3c2sc2ccccc23)cc1 ; c1ccc(-c2ccc(N(c3ccc(-c4ccc5ccc6ccccc6c5c4)cc3)c3ccc4ccc5ccccc5c4c3)c3c2sc2ccccc23)cc1] 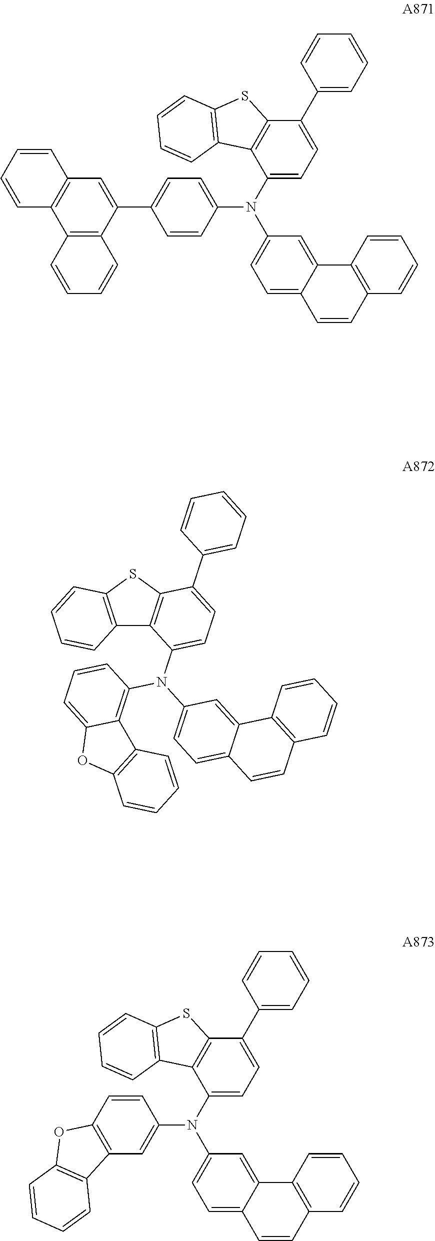 [SMILES: c1ccc(-c2ccc(N(c3ccc(-c4cc5ccccc5c5ccccc45)cc3)c3ccc4ccc5ccccc5c4c3)c3c2sc2ccccc23)cc1 ; c1ccc(-c2ccc(N(c3ccc4ccc5ccccc5c4c3)c3cccc4oc5ccccc5c34)c3c2sc2ccccc23)cc1 ; c1ccc(-c2ccc(N(c3ccc4ccc5ccccc5c4c3)c3ccc4oc5ccccc5c4c3)c3c2sc2ccccc23)cc1] 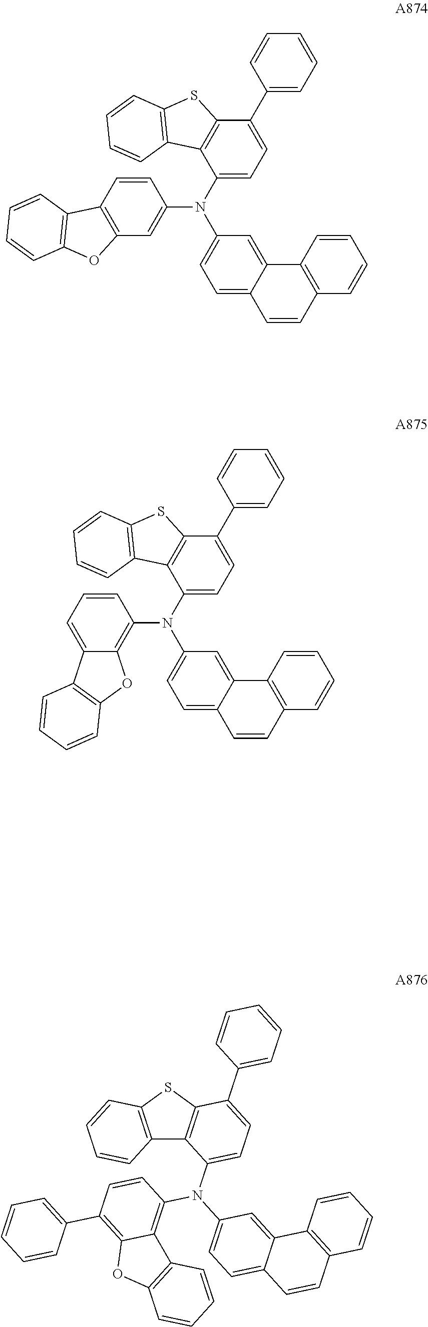 [SMILES: c1ccc(-c2ccc(N(c3ccc4c(c3)oc3ccccc34)c3ccc4ccc5ccccc5c4c3)c3c2sc2ccccc23)cc1 ; c1ccc(-c2ccc(N(c3ccc4ccc5ccccc5c4c3)c3cccc4c3oc3ccccc34)c3c2sc2ccccc23)cc1 ; c1ccc(-c2ccc(N(c3ccc4ccc5ccccc5c4c3)c3ccc(-c4ccccc4)c4sc5ccccc5c34)c3c2oc2ccccc23)cc1] 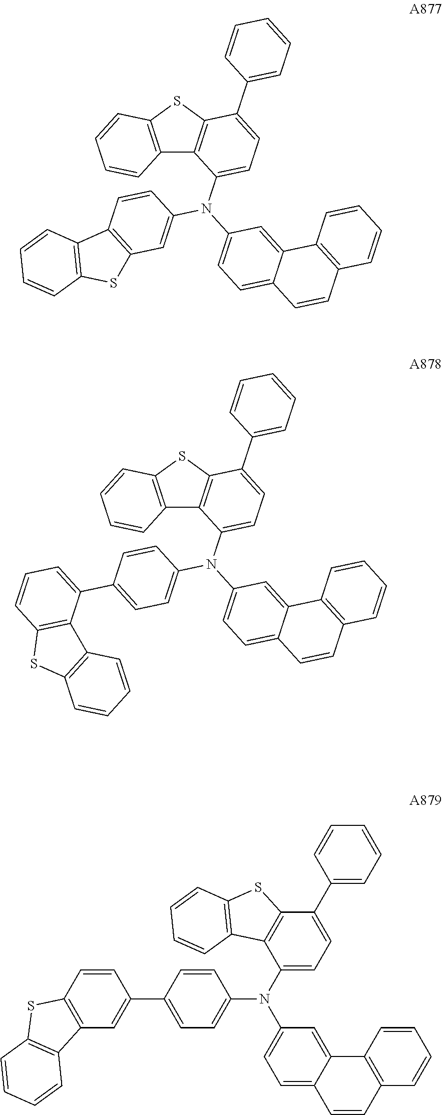 [SMILES: c1ccc(-c2ccc(N(c3ccc4c(c3)sc3ccccc34)c3ccc4ccc5ccccc5c4c3)c3c2sc2ccccc23)cc1 ; c1ccc(-c2ccc(N(c3ccc(-c4cccc5sc6ccccc6c45)cc3)c3ccc4ccc5ccccc5c4c3)c3c2sc2ccccc23)cc1 ; c1ccc(-c2ccc(N(c3ccc(-c4ccc5sc6ccccc6c5c4)cc3)c3ccc4ccc5ccccc5c4c3)c3c2sc2ccccc23)cc1] 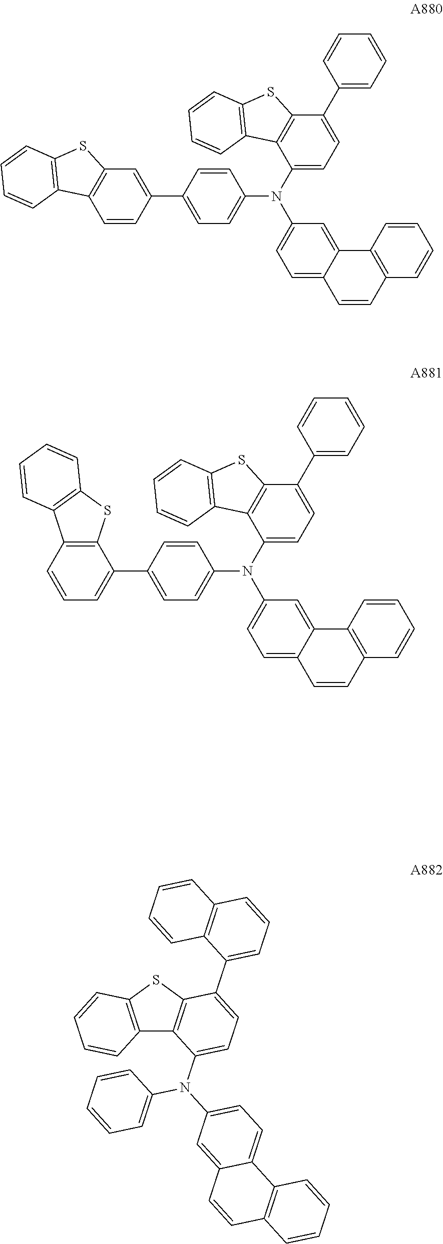 [SMILES: c1ccc(-c2ccc(N(c3ccc(-c4ccc5c(c4)sc4ccccc45)cc3)c3ccc4ccc5ccccc5c4c3)c3c2sc2ccccc23)cc1 ; c1ccc(-c2ccc(N(c3ccc(-c4cccc5c4sc4ccccc45)cc3)c3ccc4ccc5ccccc5c4c3)c3c2sc2ccccc23)cc1 ; c1ccc(N(c2ccc3c(ccc4ccccc43)c2)c2ccc(-c3cccc4ccccc34)c3sc4ccccc4c23)cc1] 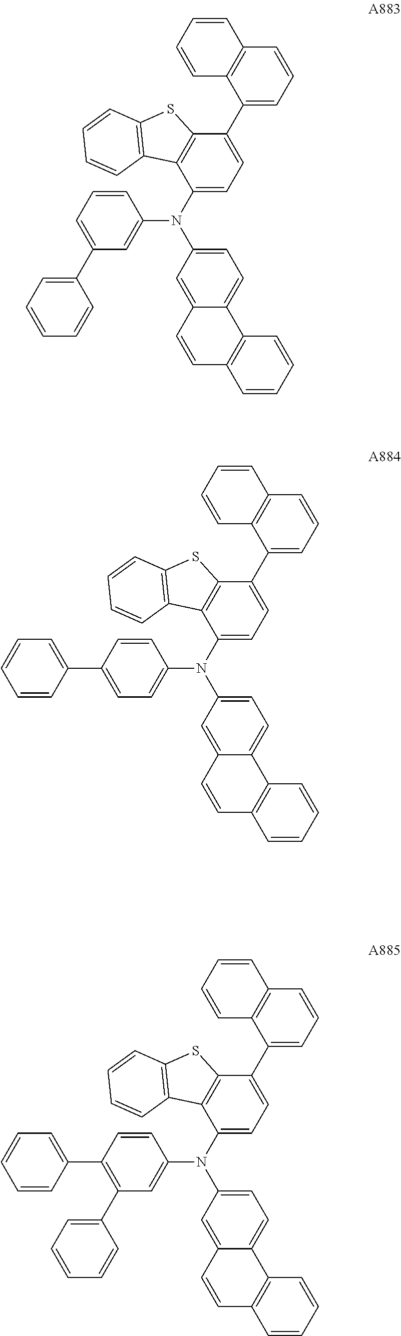 [SMILES: c1ccc(-c2cccc(N(c3ccc4c(ccc5ccccc54)c3)c3ccc(-c4cccc5ccccc45)c4sc5ccccc5c34)c2)cc1 ; c1ccc(-c2ccc(N(c3ccc4c(ccc5ccccc54)c3)c3ccc(-c4cccc5ccccc45)c4sc5ccccc5c34)cc2)cc1 ; c1ccc(-c2ccc(N(c3ccc4c(ccc5ccccc54)c3)c3ccc(-c4cccc5ccccc45)c4sc5ccccc5c34)cc2-c2ccccc2)cc1] 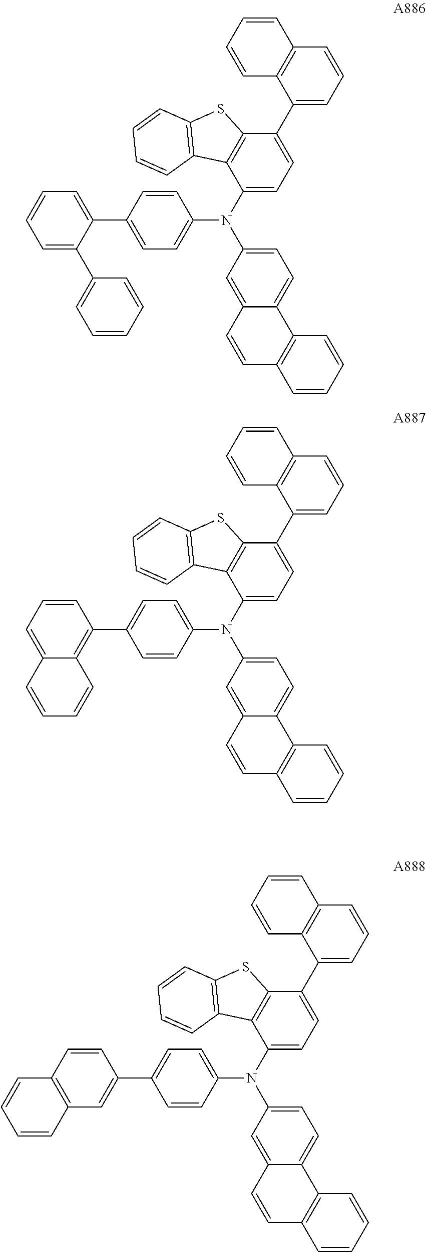 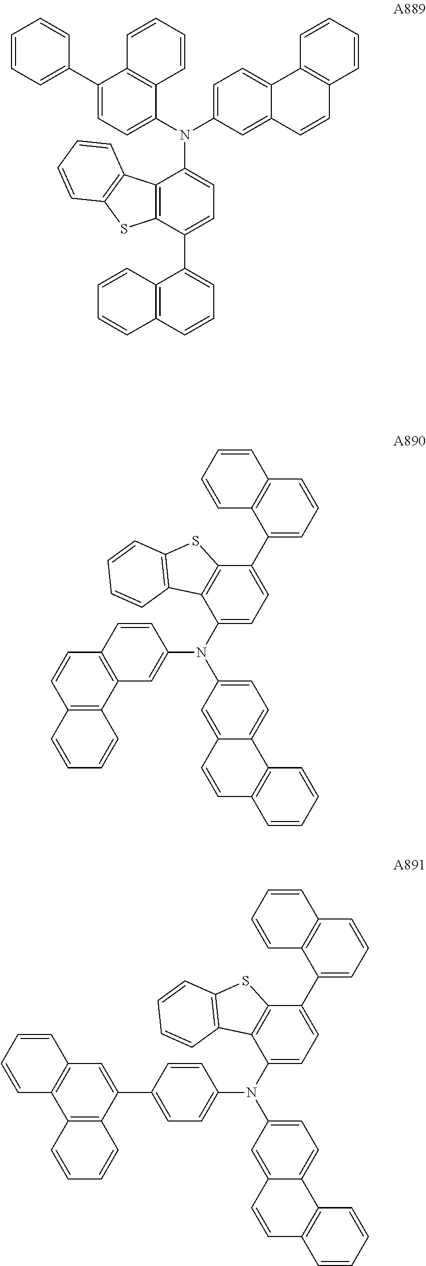 [SMILES: c1ccc(-c2ccc(N(c3ccc4c(ccc5ccccc54)c3)c3ccc(-c4cccc5ccccc45)c4sc5ccccc5c34)c3ccccc23)cc1 ; c1ccc2c(-c3ccc(N(c4ccc5c(ccc6ccccc65)c4)c4ccc5ccc6ccccc6c5c4)c4c3sc3ccccc34)cccc2c1 ; c1ccc2c(-c3ccc(N(c4ccc(-c5cc6ccccc6c6ccccc56)cc4)c4ccc5c(ccc6ccccc65)c4)c4c3sc3ccccc34)cccc2c1] 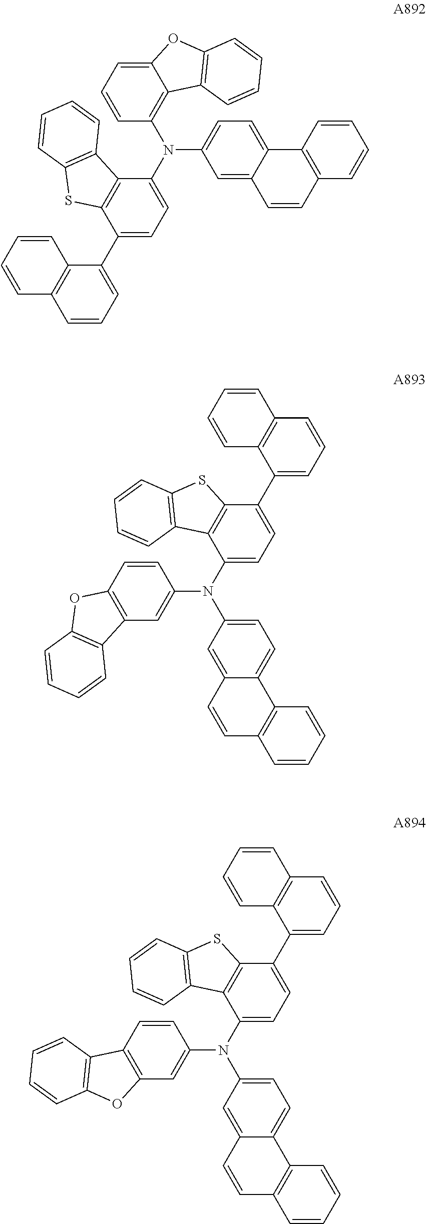 [SMILES: c1ccc2c(-c3ccc(N(c4ccc5c(ccc6ccccc65)c4)c4cccc5oc6ccccc6c45)c4c3sc3ccccc34)cccc2c1 ; c1ccc2c(-c3ccc(N(c4ccc5c(ccc6ccccc65)c4)c4ccc5oc6ccccc6c5c4)c4c3sc3ccccc34)cccc2c1 ; c1ccc2c(-c3ccc(N(c4ccc5c(ccc6ccccc65)c4)c4ccc5c(c4)oc4ccccc45)c4c3sc3ccccc34)cccc2c1] 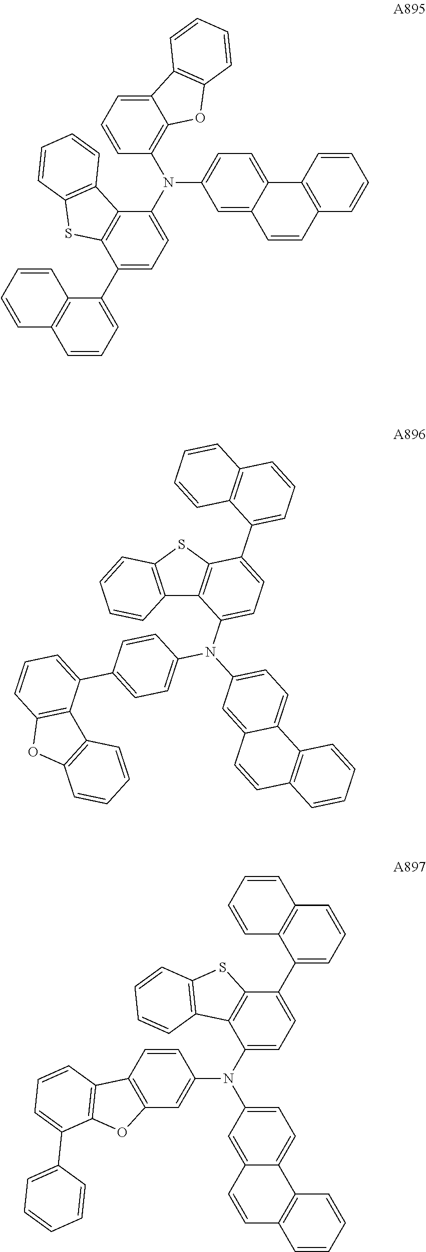 [SMILES: c1ccc2c(-c3ccc(N(c4ccc5c(ccc6ccccc65)c4)c4cccc5c4oc4ccccc45)c4c3sc3ccccc34)cccc2c1 ; c1ccc2c(-c3ccc(N(c4ccc(-c5cccc6oc7ccccc7c56)cc4)c4ccc5c(ccc6ccccc65)c4)c4c3sc3ccccc34)cccc2c1 ; c1ccc(-c2cccc3c2oc2cc(N(c4ccc5c(ccc6ccccc65)c4)c4ccc(-c5cccc6ccccc56)c5sc6ccccc6c45)ccc23)cc1] 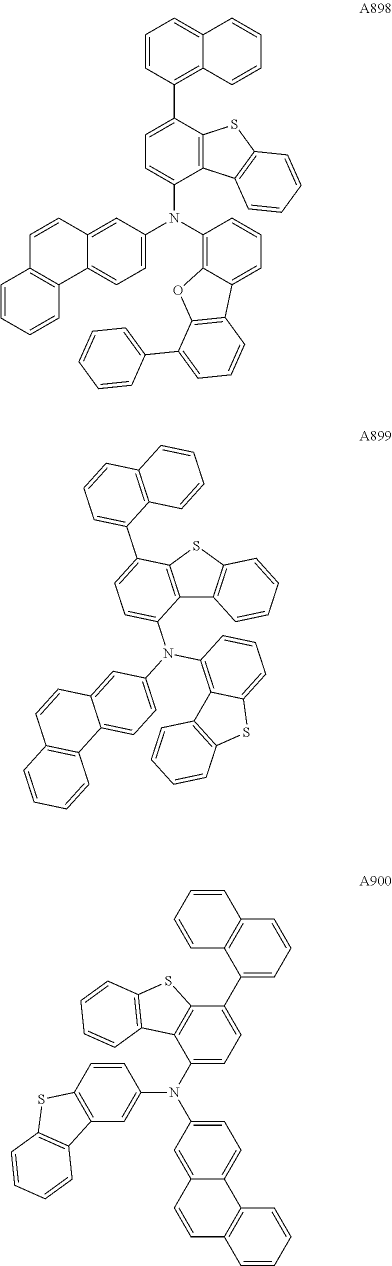 [SMILES: c1ccc(-c2cccc3c2oc2c(N(c4ccc5c(ccc6ccccc65)c4)c4ccc(-c5cccc6ccccc56)c5sc6ccccc6c45)cccc23)cc1 ; c1ccc2c(-c3ccc(N(c4ccc5c(ccc6ccccc65)c4)c4cccc5sc6ccccc6c45)c4c3sc3ccccc34)cccc2c1 ; c1ccc2c(-c3ccc(N(c4ccc5c(ccc6ccccc65)c4)c4ccc5sc6ccccc6c5c4)c4c3sc3ccccc34)cccc2c1] 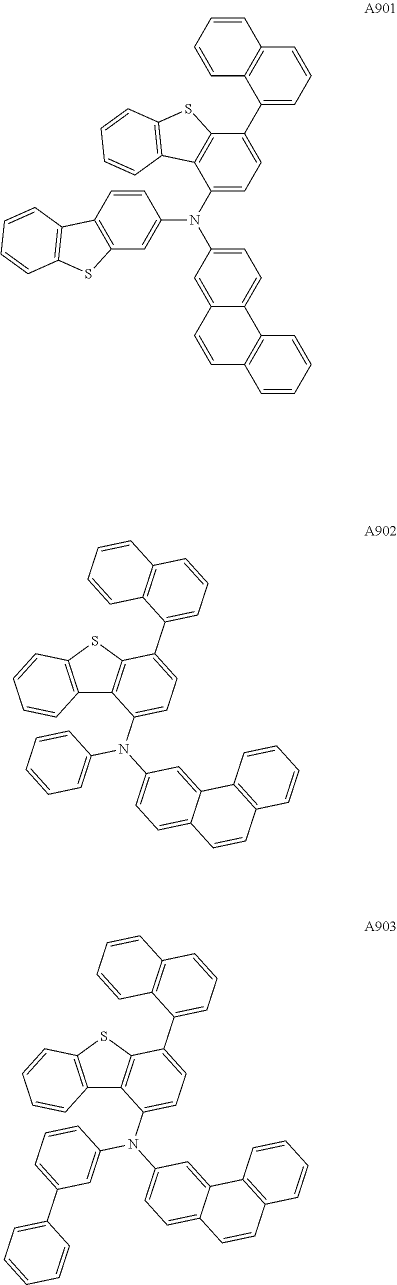 [SMILES: c1ccc2c(-c3ccc(N(c4ccc5c(ccc6ccccc65)c4)c4ccc5c(c4)sc4ccccc45)c4c3sc3ccccc34)cccc2c1 ; c1ccc(N(c2ccc3ccc4ccccc4c3c2)c2ccc(-c3cccc4ccccc34)c3sc4ccccc4c23)cc1 ; c1ccc(-c2cccc(N(c3ccc4ccc5ccccc5c4c3)c3ccc(-c4cccc5ccccc45)c4sc5ccccc5c34)c2)cc1] 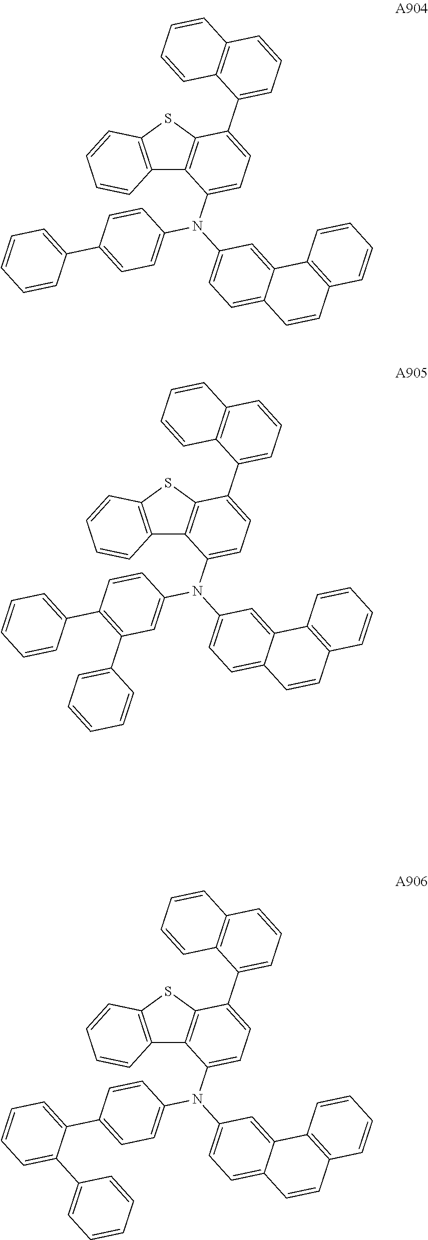 [SMILES: c1ccc(-c2ccc(N(c3ccc4ccc5ccccc5c4c3)c3ccc(-c4cccc5ccccc45)c4sc5ccccc5c34)cc2)cc1 ; c1ccc(-c2ccc(N(c3ccc4ccc5ccccc5c4c3)c3ccc(-c4cccc5ccccc45)c4sc5ccccc5c34)cc2-c2ccccc2)cc1 ; c1ccc(-c2ccccc2-c2ccc(N(c3ccc4ccc5ccccc5c4c3)c3ccc(-c4cccc5ccccc45)c4sc5ccccc5c34)cc2)cc1] 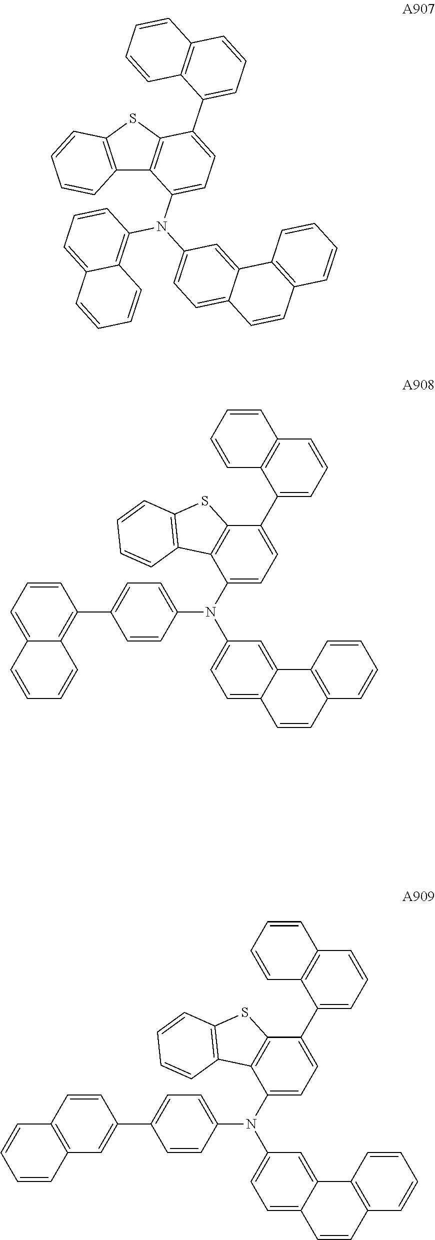 [SMILES: c1ccc2c(-c3ccc(N(c4ccc5ccc6ccccc6c5c4)c4cccc5ccccc45)c4c3sc3ccccc34)cccc2c1 ; c1ccc2c(-c3ccc(N(c4ccc5ccc6ccccc6c5c4)c4ccc(-c5cccc6ccccc56)c5sc6ccccc6c45)cc3)cccc2c1 ; c1ccc2cc(-c3ccc(N(c4ccc5ccc6ccccc6c5c4)c4ccc(-c5cccc6ccccc56)c5sc6ccccc6c45)cc3)ccc2c1] 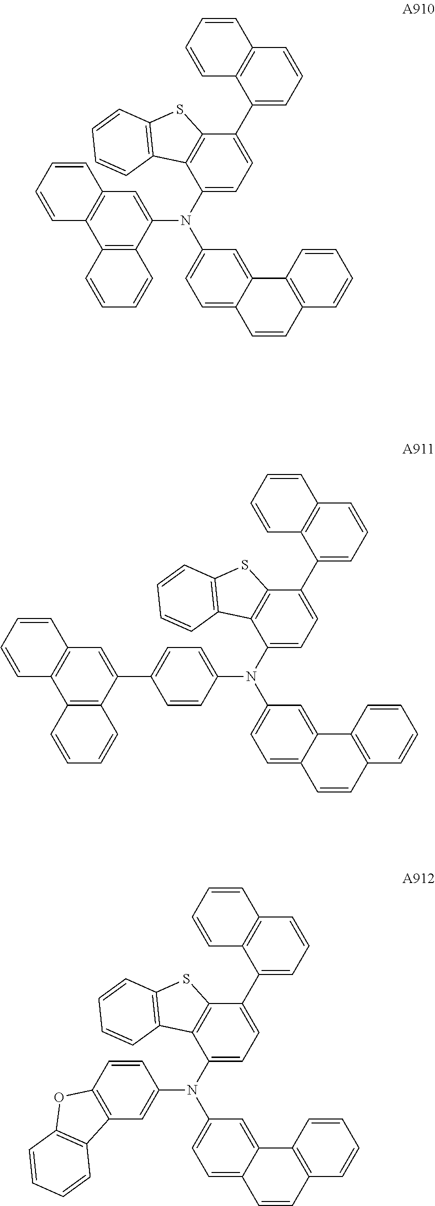 [SMILES: c1ccc2c(-c3ccc(N(c4ccc5ccc6ccccc6c5c4)c4cc5ccccc5c5ccccc45)c4c3sc3ccccc34)cccc2c1 ; c1ccc2c(-c3ccc(N(c4ccc(-c5cc6ccccc6c6ccccc56)cc4)c4ccc5ccc6ccccc6c5c4)c4c3sc3ccccc34)cccc2c1 ; c1ccc2c(-c3ccc(N(c4ccc5ccc6ccccc6c5c4)c4ccc5oc6ccccc6c5c4)c4c3sc3ccccc34)cccc2c1] 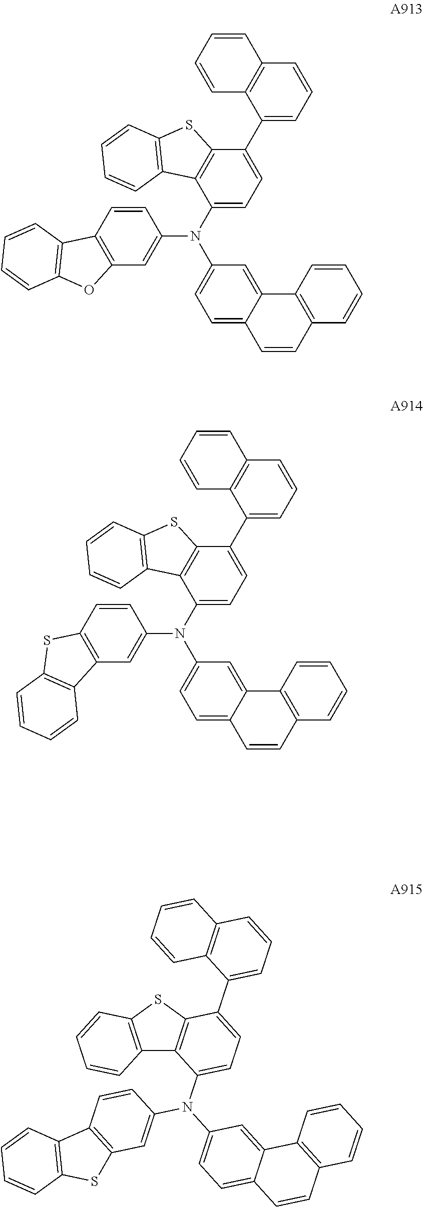 [SMILES: c1ccc2c(-c3ccc(N(c4ccc5c(c4)oc4ccccc45)c4ccc5ccc6ccccc6c5c4)c4c3sc3ccccc34)cccc2c1 ; c1ccc2c(-c3ccc(N(c4ccc5ccc6ccccc6c5c4)c4ccc5sc6ccccc6c5c4)c4c3sc3ccccc34)cccc2c1 ; c1ccc2c(-c3ccc(N(c4ccc5c(c4)sc4ccccc45)c4ccc5ccc6ccccc6c5c4)c4c3sc3ccccc34)cccc2c1] 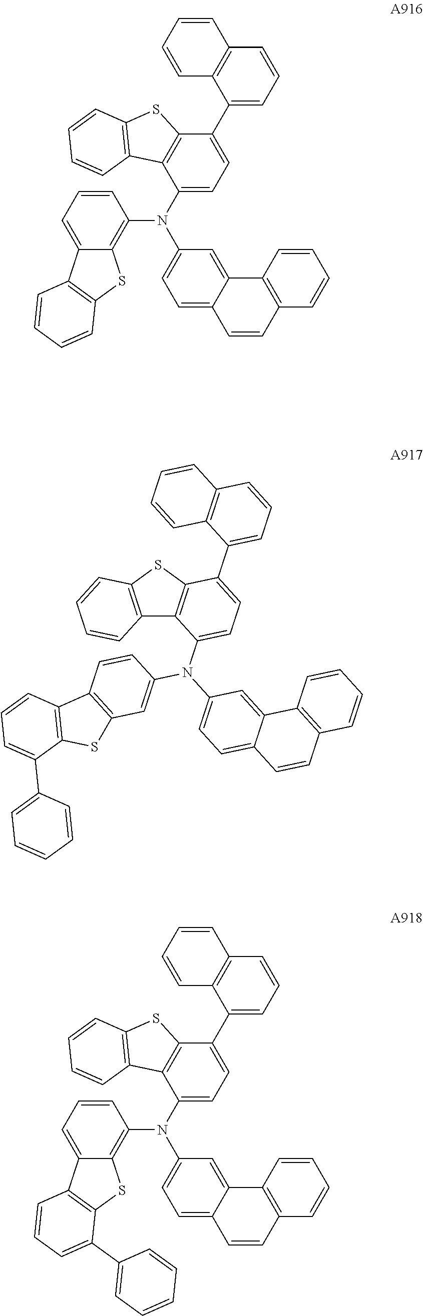 [SMILES: c1ccc2c(-c3ccc(N(c4ccc5ccc6ccccc6c5c4)c4cccc5c4sc4ccccc45)c4c3sc3ccccc34)cccc2c1 ; c1ccc(-c2cccc3c2sc2cc(N(c4ccc5ccc6ccccc6c5c4)c4ccc(-c5cccc6ccccc56)c5sc6ccccc6c45)ccc23)cc1 ; c1ccc(-c2cccc3c2sc2c(N(c4ccc5ccc6ccccc6c5c4)c4ccc(-c5cccc6ccccc56)c5sc6ccccc6c45)cccc23)cc1] 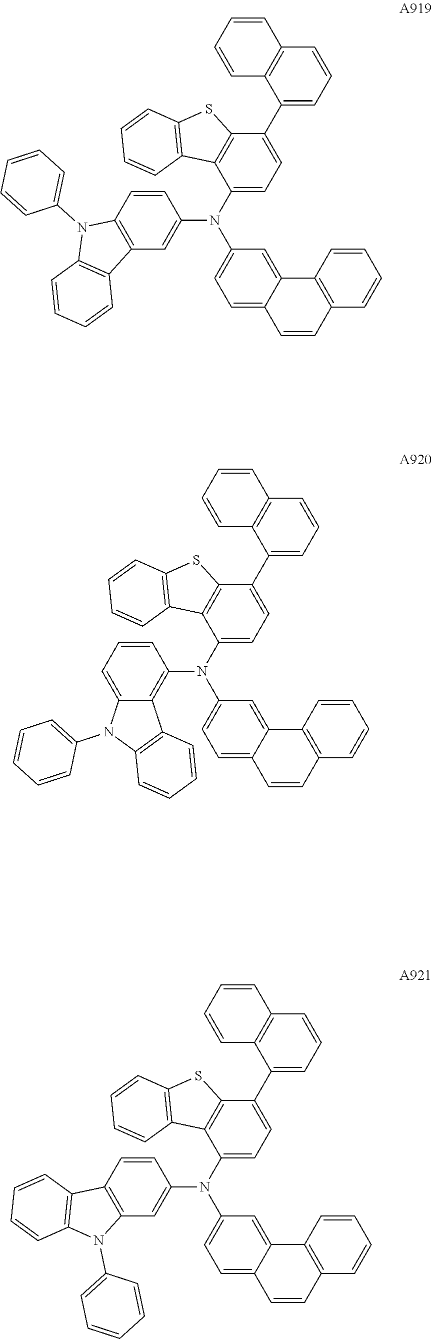 [SMILES: c1ccc(-n2c3ccccc3c3cc(N(c4ccc5ccc6ccccc6c5c4)c4ccc(-c5cccc6ccccc56)c5sc6ccccc6c45)ccc32)cc1 ; c1ccc(-n2c3ccccc3c3c(N(c4ccc5ccc6ccccc6c5c4)c4ccc(-c5cccc6ccccc56)c5sc6ccccc6c45)cccc32)cc1 ; c1ccc(-n2c3ccccc3c3ccc(N(c4ccc5ccc6ccccc6c5c4)c4ccc(-c5cccc6ccccc56)c5sc6ccccc6c45)cc32)cc1] 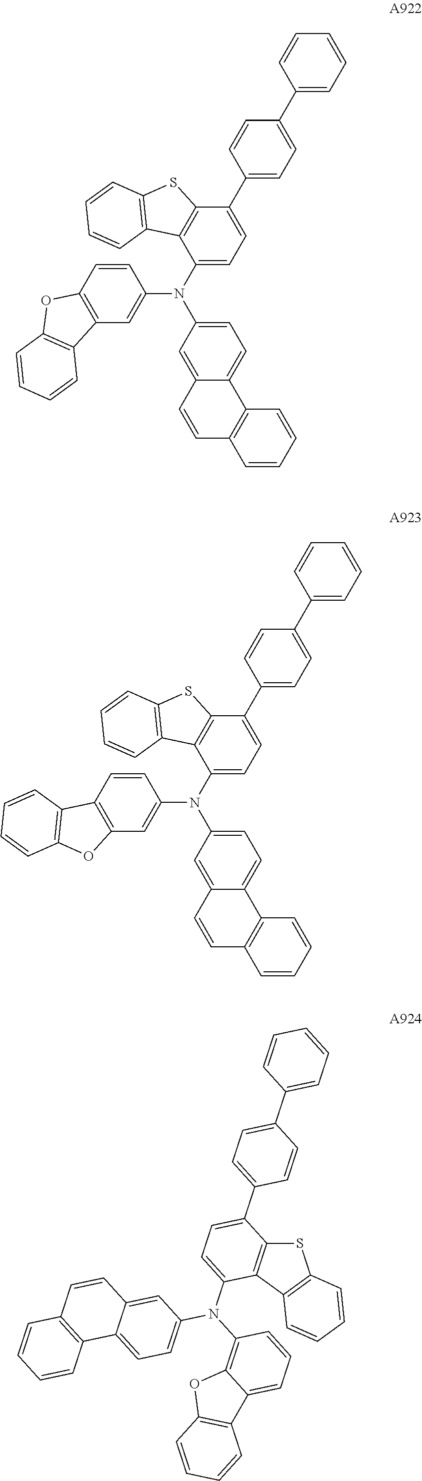 [SMILES: c1ccc(-c2ccc(-c3ccc(N(c4ccc5c(ccc6ccccc65)c4)c4ccc5oc6ccccc6c5c4)c4c3sc3ccccc34)cc2)cc1 ; c1ccc(-c2ccc(-c3ccc(N(c4ccc5c(ccc6ccccc65)c4)c4ccc5c(c4)oc4ccccc45)c4c3sc3ccccc34)cc2)cc1 ; c1ccc(-c2ccc(-c3ccc(N(c4ccc5c(ccc6ccccc65)c4)c4cccc5c4oc4ccccc45)c4c3sc3ccccc34)cc2)cc1] 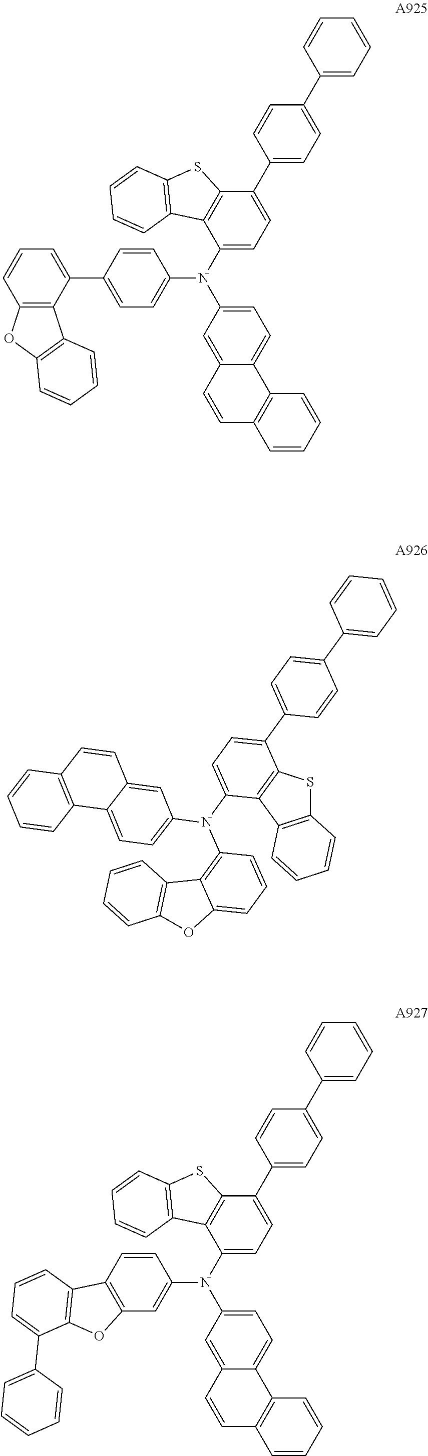 [SMILES: c1ccc(-c2ccc(-c3ccc(N(c4ccc(-c5cccc6oc7ccccc7c56)cc4)c4ccc5c(ccc6ccccc65)c4)c4c3sc3ccccc34)cc2)cc1 ; c1ccc(-c2ccc(-c3ccc(N(c4ccc5c(ccc6ccccc65)c4)c4cccc5oc6ccccc6c45)c4c3sc3ccccc34)cc2)cc1 ; c1ccc(-c2ccc(-c3ccc(N(c4ccc5c(ccc6ccccc65)c4)c4ccc5c(c4)oc4c(-c6ccccc6)cccc45)c4c3sc3ccccc34)cc2)cc1] 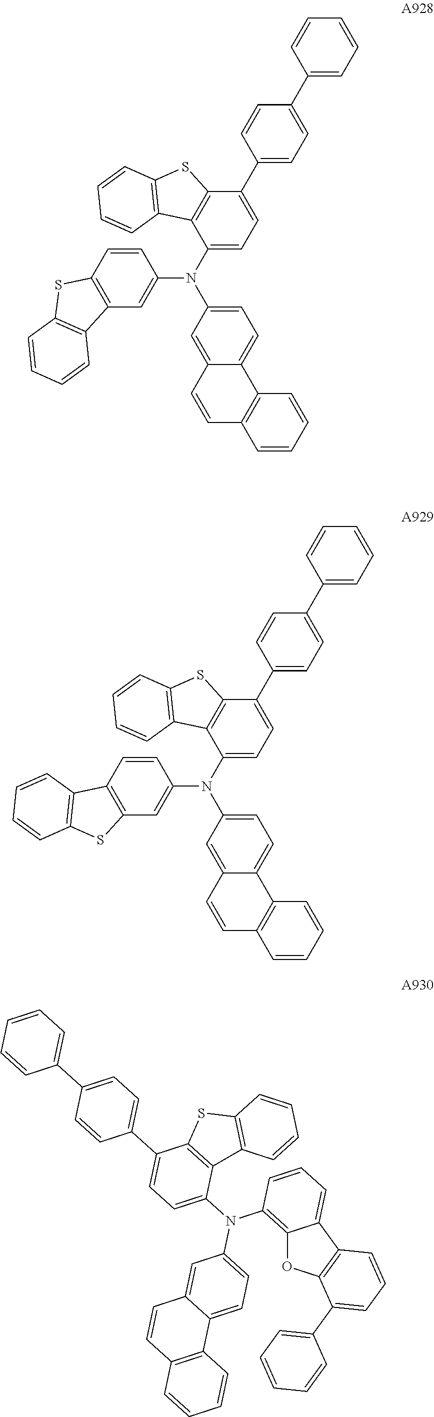 [SMILES: c1ccc(-c2ccc(-c3ccc(N(c4ccc5c(ccc6ccccc65)c4)c4ccc5sc6ccccc6c5c4)c4c3sc3ccccc34)cc2)cc1 ; c1ccc(-c2ccc(-c3ccc(N(c4ccc5c(ccc6ccccc65)c4)c4ccc5c(c4)sc4ccccc45)c4c3sc3ccccc34)cc2)cc1 ; c1ccc(-c2ccc(-c3ccc(N(c4ccc5c(ccc6ccccc65)c4)c4cccc5c4oc4c(-c6ccccc6)cccc45)c4c3sc3ccccc34)cc2)cc1] 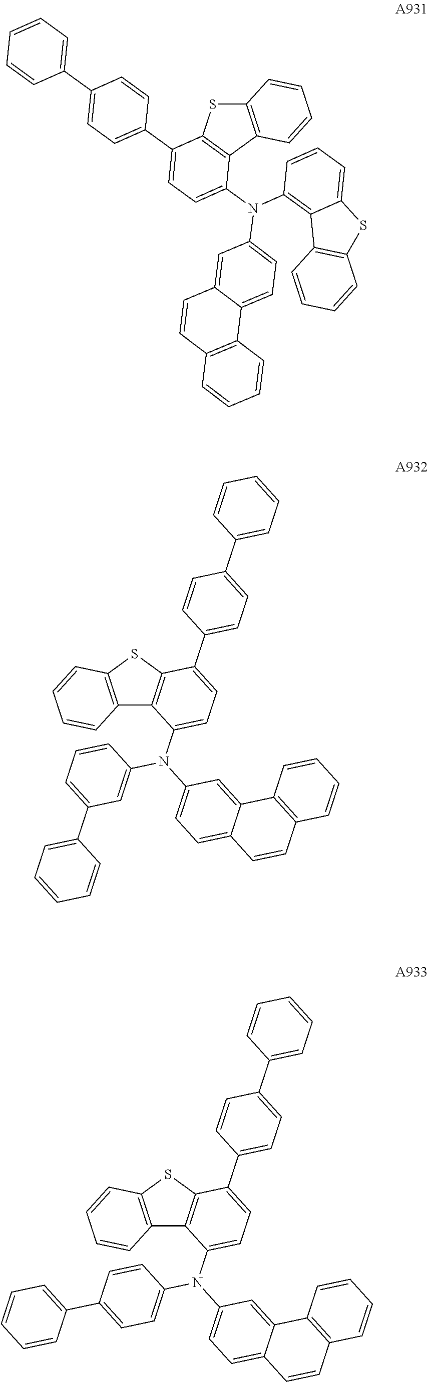 [SMILES: c1ccc(-c2ccc(-c3ccc(N(c4ccc5c(ccc6ccccc65)c4)c4cccc5sc6ccccc6c45)c4c3sc3ccccc34)cc2)cc1 ; c1ccc(-c2ccc(-c3ccc(N(c4cccc(-c5ccccc5)c4)c4ccc5ccc6ccccc6c5c4)c4c3sc3ccccc34)cc2)cc1 ; c1ccc(-c2ccc(-c3ccc(N(c4ccc(-c5ccccc5)cc4)c4ccc5ccc6ccccc6c5c4)c4c3sc3ccccc34)cc2)cc1] 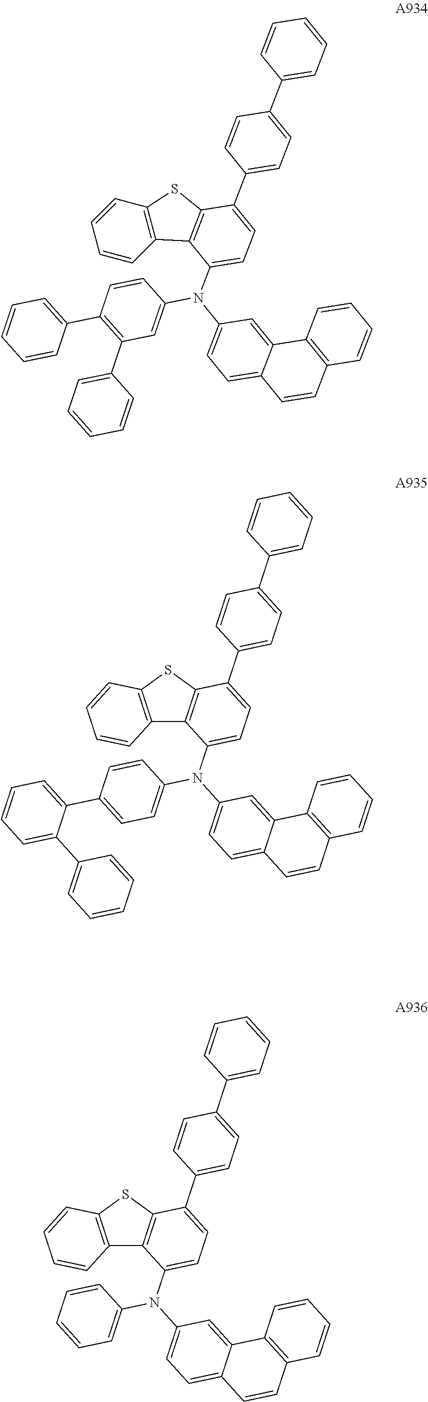 [SMILES: c1ccc(-c2ccc(-c3ccc(N(c4ccc(-c5ccccc5)c(-c5ccccc5)c4)c4ccc5ccc6ccccc6c5c4)c4c3sc3ccccc34)cc2)cc1 ; c1ccc(-c2ccc(-c3ccc(N(c4ccc(-c5ccccc5-c5ccccc5)cc4)c4ccc5ccc6ccccc6c5c4)c4c3sc3ccccc34)cc2)cc1 ; c1ccc(-c2ccc(-c3ccc(N(c4ccccc4)c4ccc5ccc6ccccc6c5c4)c4c3sc3ccccc34)cc2)cc1] 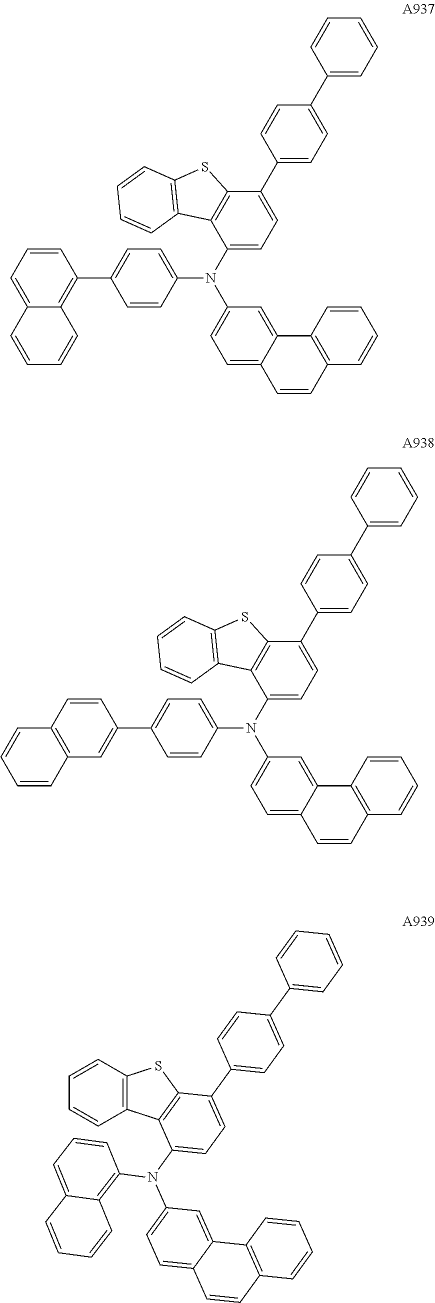 [SMILES: c1ccc(-c2ccc(-c3ccc(N(c4ccc(-c5cccc6ccccc56)cc4)c4ccc5ccc6ccccc6c5c4)c4c3sc3ccccc34)cc2)cc1 ; c1ccc(-c2ccc(-c3ccc(N(c4ccc(-c5ccc6ccccc6c5)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5ccccc45)c4c3sc3ccccc34)cc2)cc1] 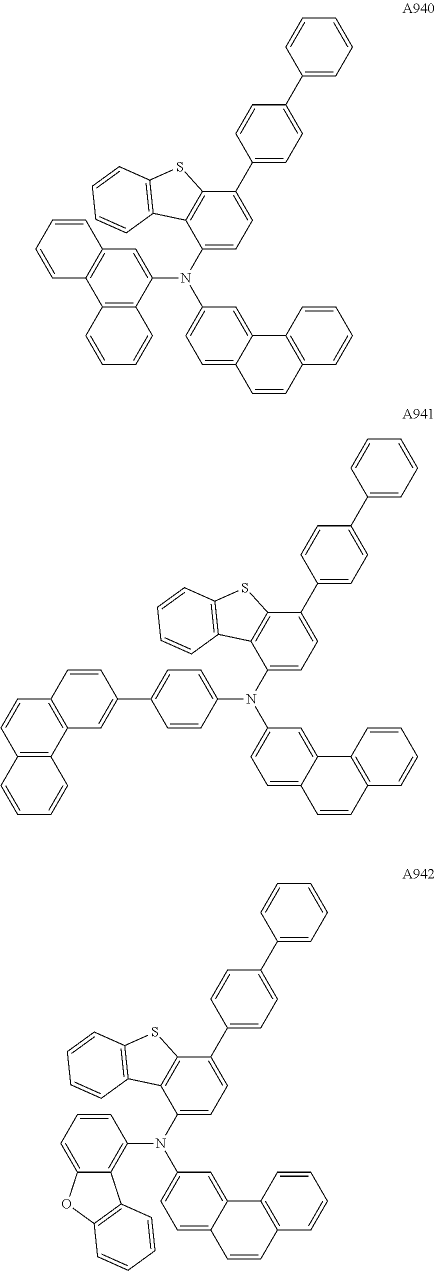 [SMILES: c1ccc(-c2ccc(-c3ccc(N(c4ccc5ccc6ccccc6c5c4)c4cc5ccccc5c5ccccc45)c4c3sc3ccccc34)cc2)cc1 ; c1ccc(-c2ccc(-c3ccc(N(c4ccc(-c5ccc6ccc7ccccc7c6c5)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5oc6ccccc6c45)c4c3sc3ccccc34)cc2)cc1] 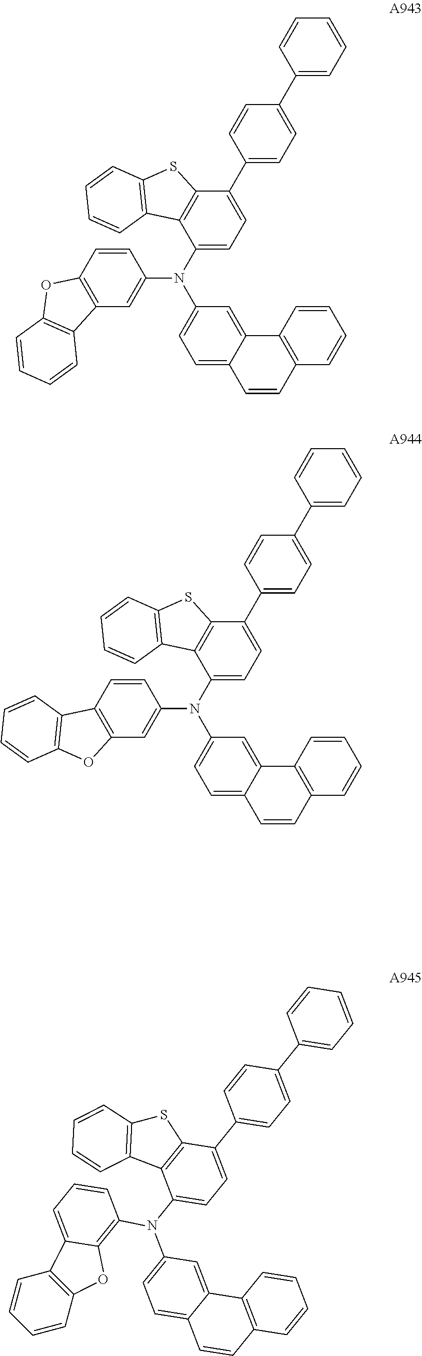 [SMILES: c1ccc(-c2ccc(-c3ccc(N(c4ccc5ccc6ccccc6c5c4)c4ccc5oc6ccccc6c5c4)c4c3sc3ccccc34)cc2)cc1 ; c1ccc(-c2ccc(-c3ccc(N(c4ccc5c(c4)oc4ccccc45)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5c4oc4ccccc45)c4c3sc3ccccc34)cc2)cc1] 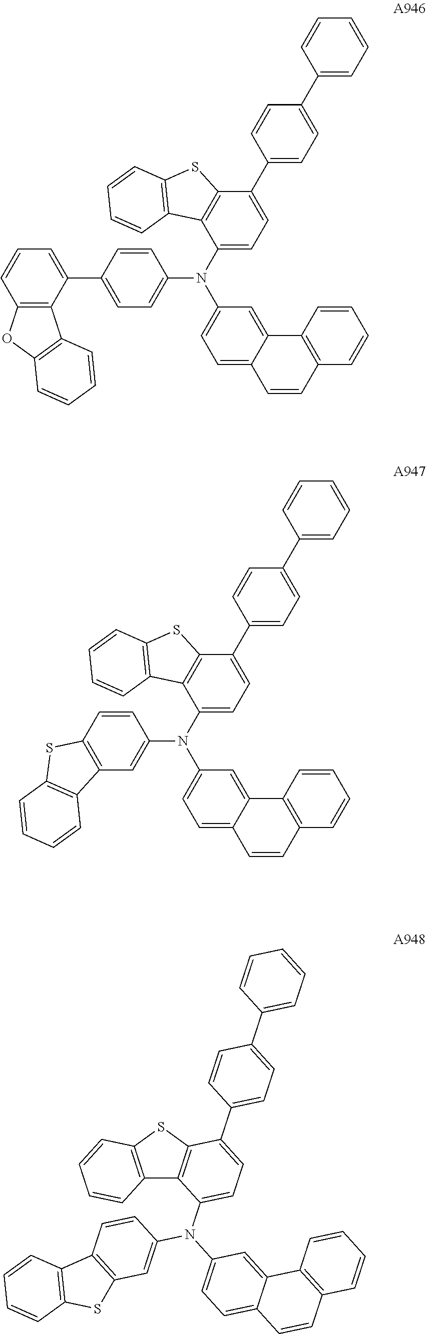 [SMILES: c1ccc(-c2ccc(-c3ccc(N(c4ccc(-c5cccc6oc7ccccc7c56)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4ccc5sc6ccccc6c5c4)c4c3sc3ccccc34)cc2)cc1 ; c1ccc(-c2ccc(-c3ccc(N(c4ccc5c(c4)sc4ccccc45)c4ccc5ccc6ccccc6c5c4)c4c3sc3ccccc34)cc2)cc1] 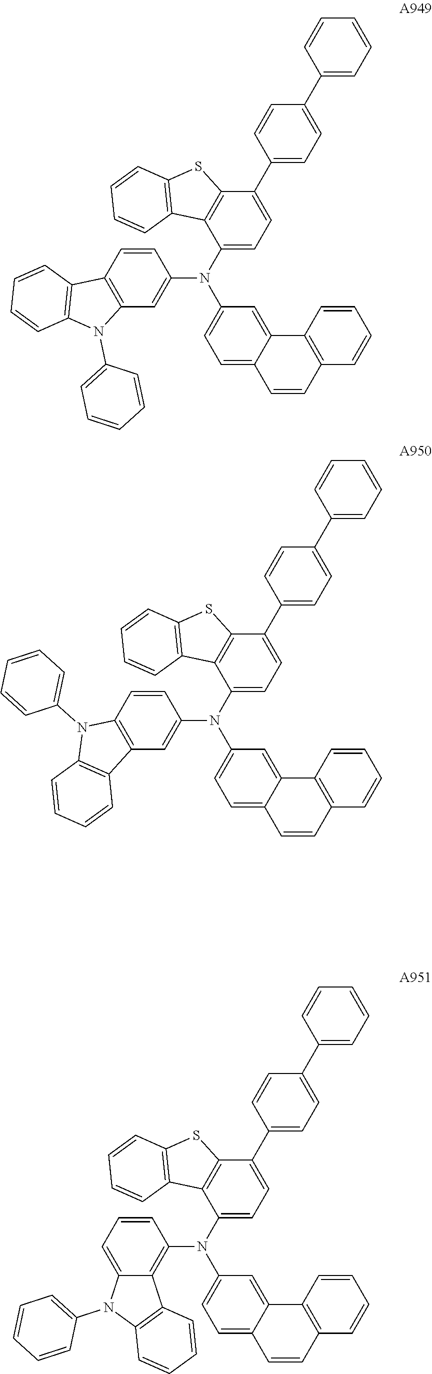 [SMILES: c1ccc(-c2ccc(-c3ccc(N(c4ccc5ccc6ccccc6c5c4)c4ccc5c6ccccc6n(-c6ccccc6)c5c4)c4c3sc3ccccc34)cc2)cc1 ; c1ccc(-c2ccc(-c3ccc(N(c4ccc5ccc6ccccc6c5c4)c4ccc5c(c4)c4ccccc4n5-c4ccccc4)c4c3sc3ccccc34)cc2)cc1 ; c1ccc(-c2ccc(-c3ccc(N(c4ccc5ccc6ccccc6c5c4)c4cccc5c4c4ccccc4n5-c4ccccc4)c4c3sc3ccccc34)cc2)cc1] 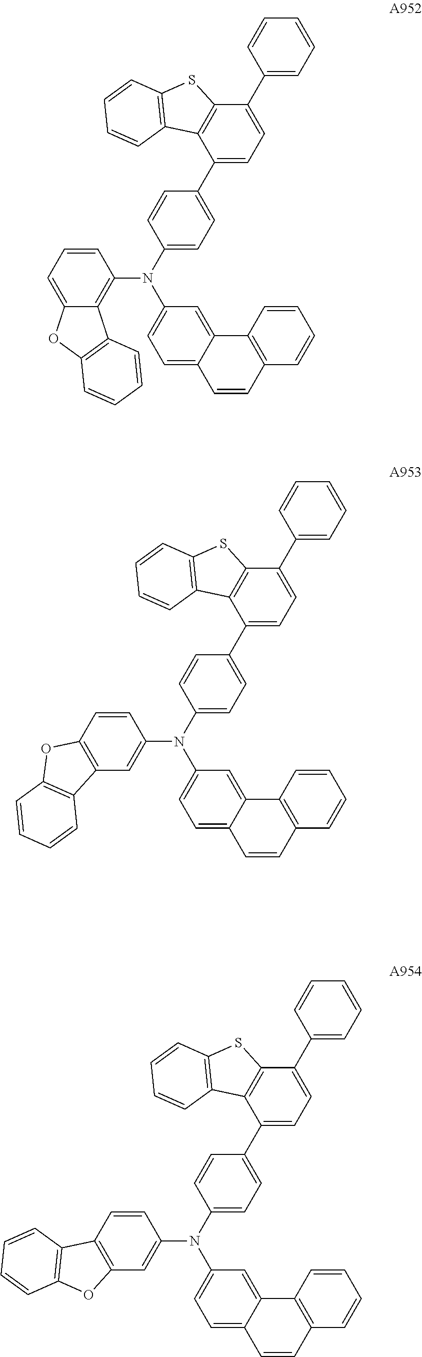 [SMILES: c1ccc(-c2ccc(-c3ccc(N(c4ccc5ccc6ccccc6c5c4)c4cccc5oc6ccccc6c45)cc3)c3c2sc2ccccc23)cc1 ; c1ccc(-c2ccc(-c3ccc(N(c4ccc5ccc6ccccc6c5c4)c4ccc5oc6ccccc6c5c4)cc3)c3c2sc2ccccc23)cc1 ; c1ccc(-c2ccc(-c3ccc(N(c4ccc5c(c4)oc4ccccc45)c4ccc5ccc6ccccc6c5c4)cc3)c3c2sc2ccccc23)cc1] 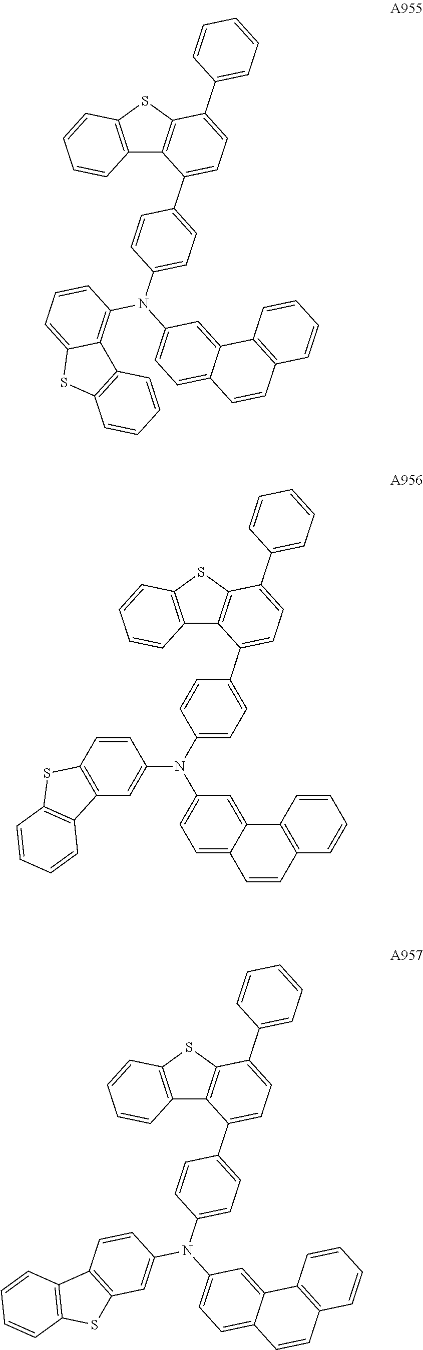 [SMILES: c1ccc(-c2ccc(-c3ccc(N(c4ccc5ccc6ccccc6c5c4)c4cccc5sc6ccccc6c45)cc3)c3c2sc2ccccc23)cc1 ; c1ccc(-c2ccc(-c3ccc(N(c4ccc5ccc6ccccc6c5c4)c4ccc5sc6ccccc6c5c4)cc3)c3c2sc2ccccc23)cc1 ; c1ccc(-c2ccc(-c3ccc(N(c4ccc5c(c4)sc4ccccc45)c4ccc5ccc6ccccc6c5c4)cc3)c3c2sc2ccccc23)cc1] 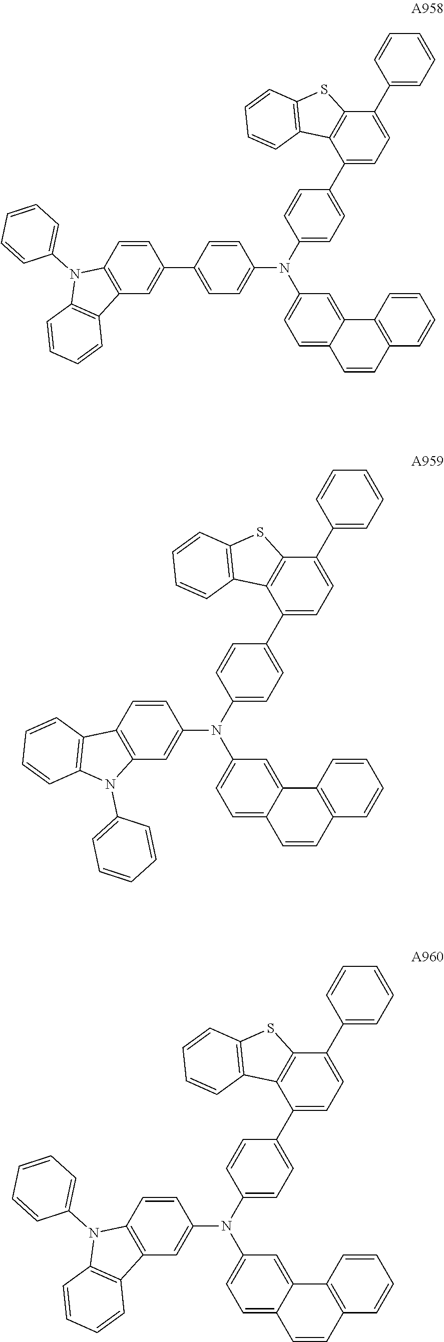 [SMILES: c1ccc(-c2ccc(-c3ccc(N(c4ccc(-c5ccc6c(c5)c5ccccc5n6-c5ccccc5)cc4)c4ccc5ccc6ccccc6c5c4)cc3)c3c2sc2ccccc23)cc1 ; c1ccc(-c2ccc(-c3ccc(N(c4ccc5ccc6ccccc6c5c4)c4ccc5c6ccccc6n(-c6ccccc6)c5c4)cc3)c3c2sc2ccccc23)cc1 ; c1ccc(-c2ccc(-c3ccc(N(c4ccc5ccc6ccccc6c5c4)c4ccc5c(c4)c4ccccc4n5-c4ccccc4)cc3)c3c2sc2ccccc23)cc1] 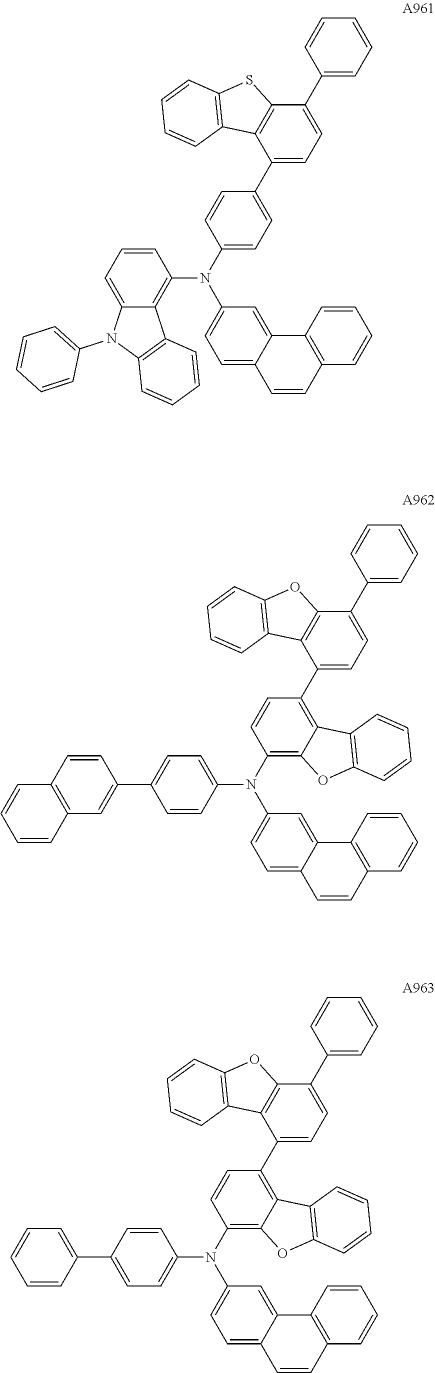 [SMILES: c1ccc(-c2ccc(-c3ccc(N(c4ccc5ccc6ccccc6c5c4)c4cccc5c4c4ccccc4n5-c4ccccc4)cc3)c3c2sc2ccccc23)cc1 ; c1ccc(-c2ccc(-c3ccc(N(c4ccc(-c5ccc6ccccc6c5)cc4)c4ccc5ccc6ccccc6c5c4)c4oc5ccccc5c34)c3c2oc2ccccc23)cc1 ; c1ccc(-c2ccc(N(c3ccc4ccc5ccccc5c4c3)c3ccc(-c4ccc(-c5ccccc5)c5oc6ccccc6c45)c4c3oc3ccccc34)cc2)cc1] 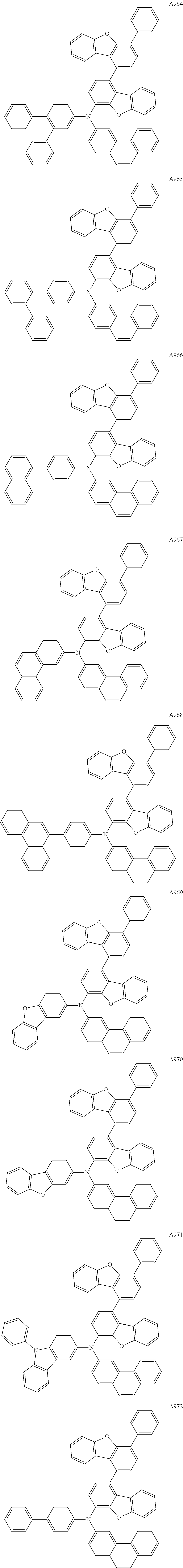 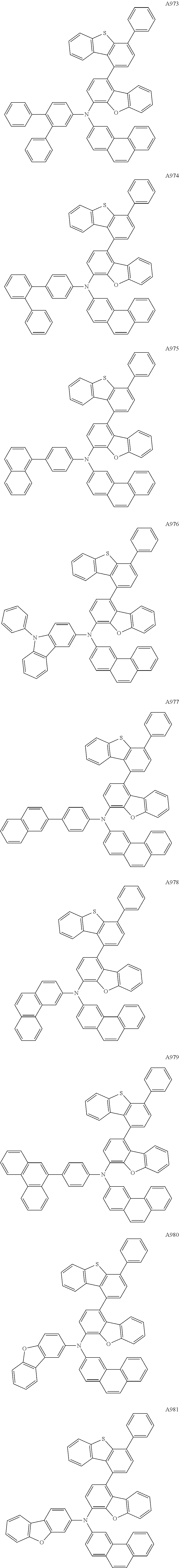 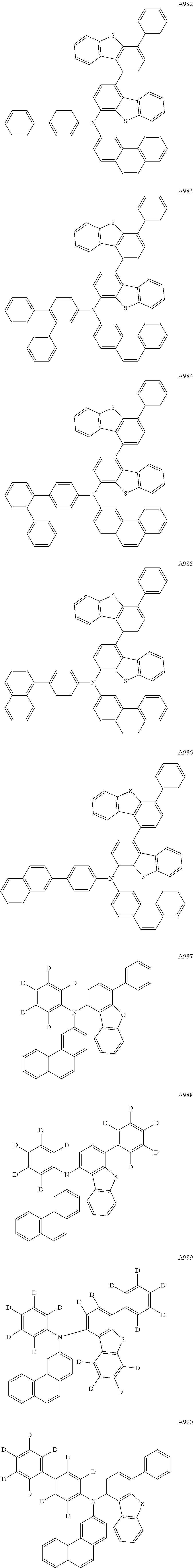 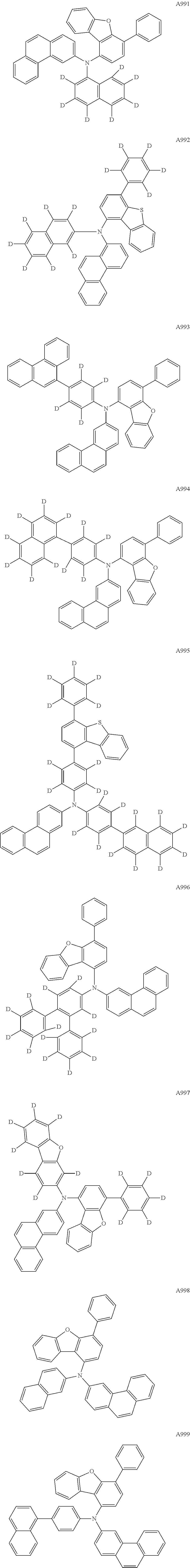 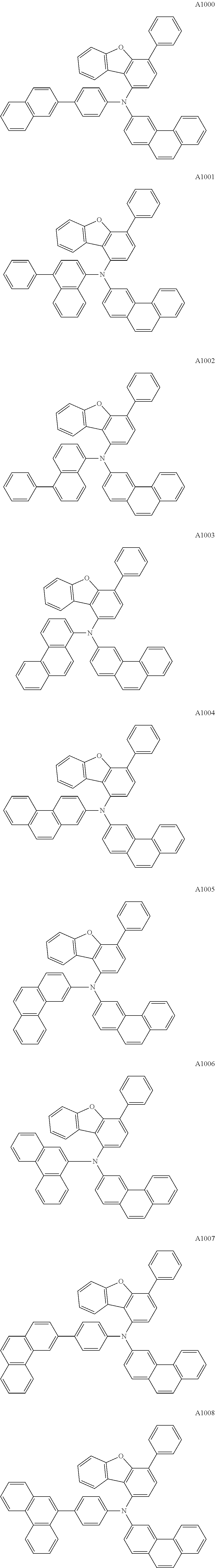 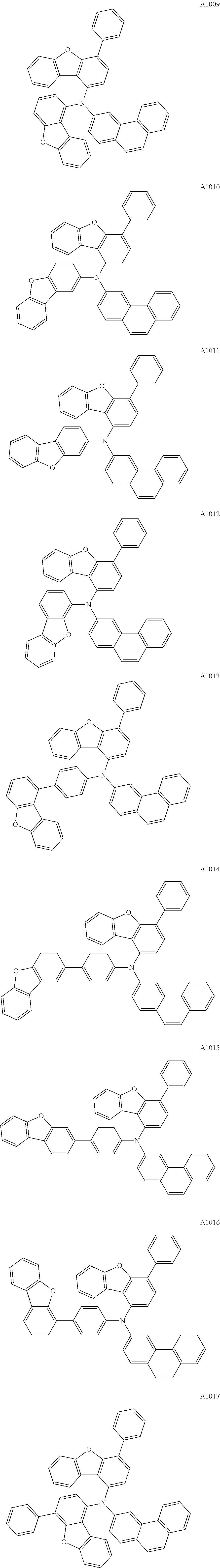 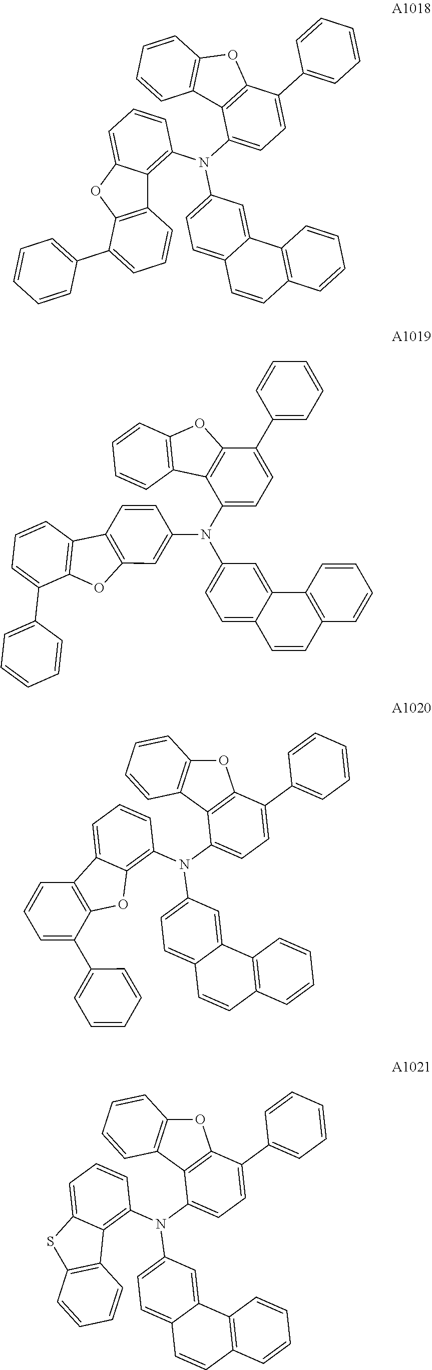 [SMILES: c1ccc(-c2cccc3c2oc2cccc(N(c4ccc5ccc6ccccc6c5c4)c4ccc(-c5ccccc5)c5oc6ccccc6c45)c23)cc1 ; c1ccc(-c2cccc3c2oc2cc(N(c4ccc5ccc6ccccc6c5c4)c4ccc(-c5ccccc5)c5oc6ccccc6c45)ccc23)cc1 ; c1ccc(-c2cccc3c2oc2c(N(c4ccc5ccc6ccccc6c5c4)c4ccc(-c5ccccc5)c5oc6ccccc6c45)cccc23)cc1 ; c1ccc(-c2ccc(N(c3ccc4ccc5ccccc5c4c3)c3cccc4sc5ccccc5c34)c3c2oc2ccccc23)cc1] 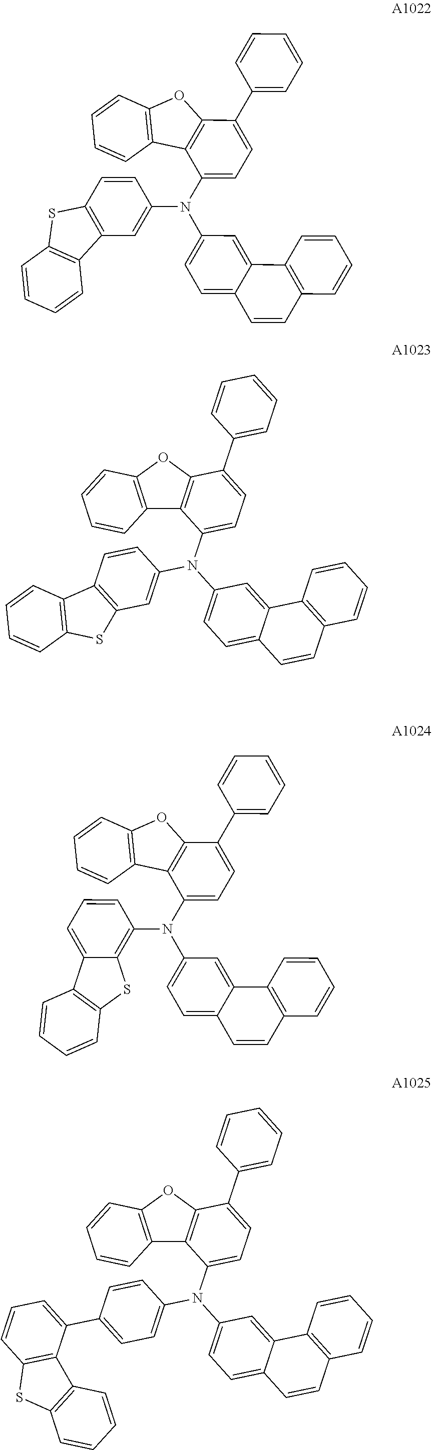 [SMILES: c1ccc(-c2ccc(N(c3ccc4ccc5ccccc5c4c3)c3ccc4sc5ccccc5c4c3)c3c2oc2ccccc23)cc1 ; c1ccc(-c2ccc(N(c3ccc4c(c3)sc3ccccc34)c3ccc4ccc5ccccc5c4c3)c3c2oc2ccccc23)cc1 ; c1ccc(-c2ccc(N(c3ccc4ccc5ccccc5c4c3)c3cccc4c3sc3ccccc34)c3c2oc2ccccc23)cc1 ; c1ccc(-c2ccc(N(c3ccc(-c4cccc5sc6ccccc6c45)cc3)c3ccc4ccc5ccccc5c4c3)c3c2oc2ccccc23)cc1] 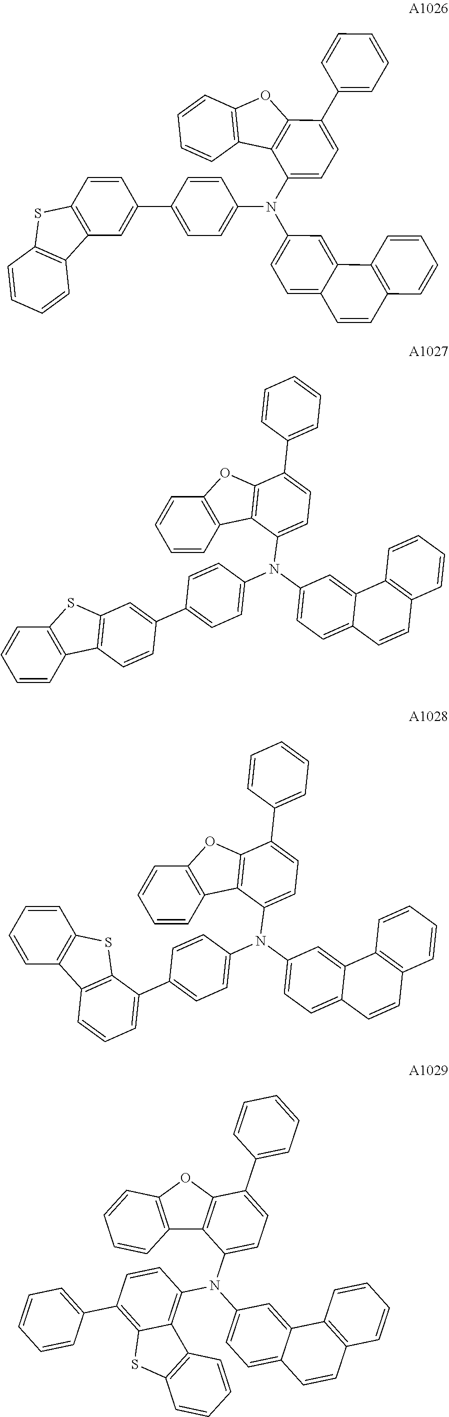 [SMILES: c1ccc(-c2ccc(N(c3ccc(-c4ccc5sc6ccccc6c5c4)cc3)c3ccc4ccc5ccccc5c4c3)c3c2oc2ccccc23)cc1 ; c1ccc(-c2ccc(N(c3ccc(-c4ccc5c(c4)sc4ccccc45)cc3)c3ccc4ccc5ccccc5c4c3)c3c2oc2ccccc23)cc1 ; c1ccc(-c2ccc(N(c3ccc(-c4cccc5c4sc4ccccc45)cc3)c3ccc4ccc5ccccc5c4c3)c3c2oc2ccccc23)cc1 ; c1ccc(-c2ccc(N(c3ccc4ccc5ccccc5c4c3)c3ccc(-c4ccccc4)c4sc5ccccc5c34)c3c2oc2ccccc23)cc1] 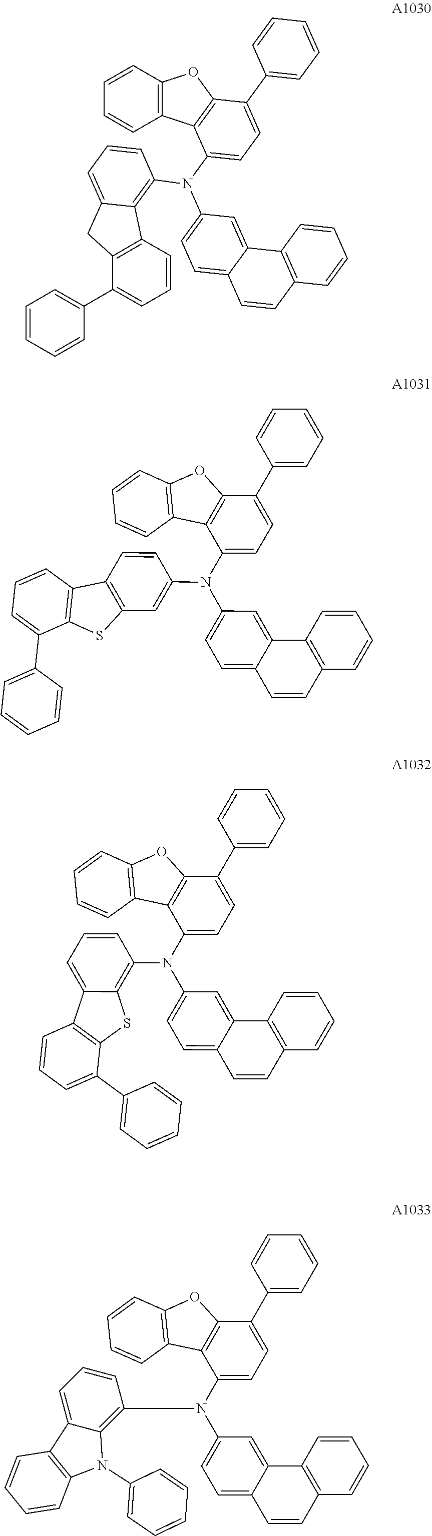 [SMILES: c1ccc(-c2cccc3c2Cc2cccc(N(c4ccc5ccc6ccccc6c5c4)c4ccc(-c5ccccc5)c5oc6ccccc6c45)c2-3)cc1 ; c1ccc(-c2ccc(N(c3ccc4c(c3)sc3c(-c5ccccc5)cccc34)c3ccc4ccc5ccccc5c4c3)c3c2oc2ccccc23)cc1 ; c1ccc(-c2ccc(N(c3ccc4ccc5ccccc5c4c3)c3cccc4c3sc3c(-c5ccccc5)cccc34)c3c2oc2ccccc23)cc1 ; c1ccc(-c2ccc(N(c3ccc4ccc5ccccc5c4c3)c3cccc4c5ccccc5n(-c5ccccc5)c34)c3c2oc2ccccc23)cc1] 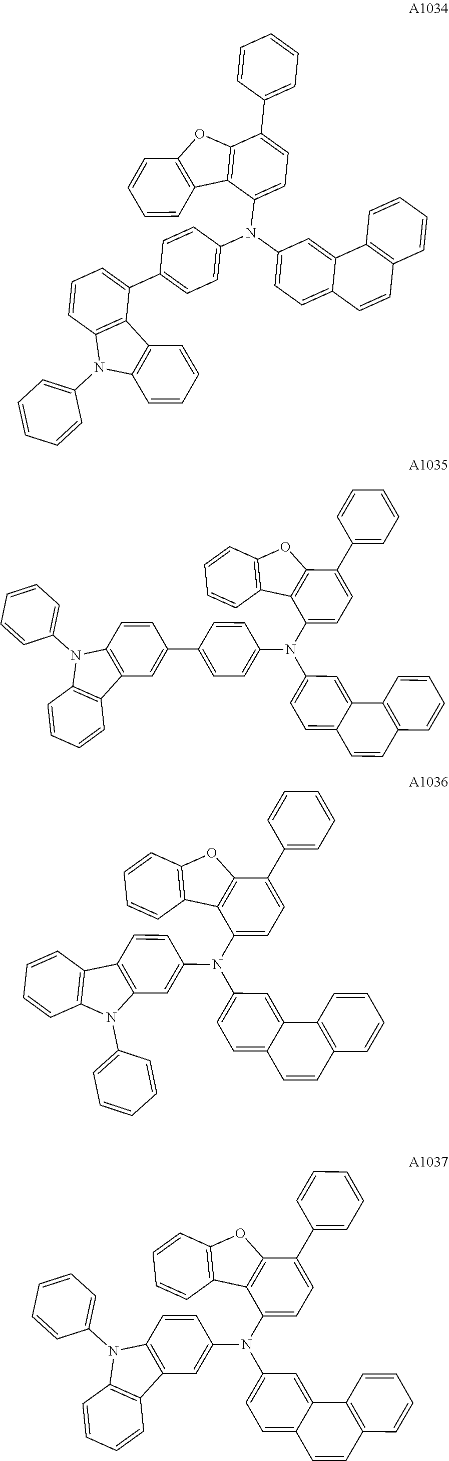 [SMILES: c1ccc(-c2ccc(N(c3ccc(-c4cccc5c4c4ccccc4n5-c4ccccc4)cc3)c3ccc4ccc5ccccc5c4c3)c3c2oc2ccccc23)cc1 ; c1ccc(-c2ccc(N(c3ccc(-c4ccc5c(c4)c4ccccc4n5-c4ccccc4)cc3)c3ccc4ccc5ccccc5c4c3)c3c2oc2ccccc23)cc1 ; c1ccc(-c2ccc(N(c3ccc4ccc5ccccc5c4c3)c3ccc4c5ccccc5n(-c5ccccc5)c4c3)c3c2oc2ccccc23)cc1 ; c1ccc(-c2ccc(N(c3ccc4ccc5ccccc5c4c3)c3ccc4c(c3)c3ccccc3n4-c3ccccc3)c3c2oc2ccccc23)cc1] 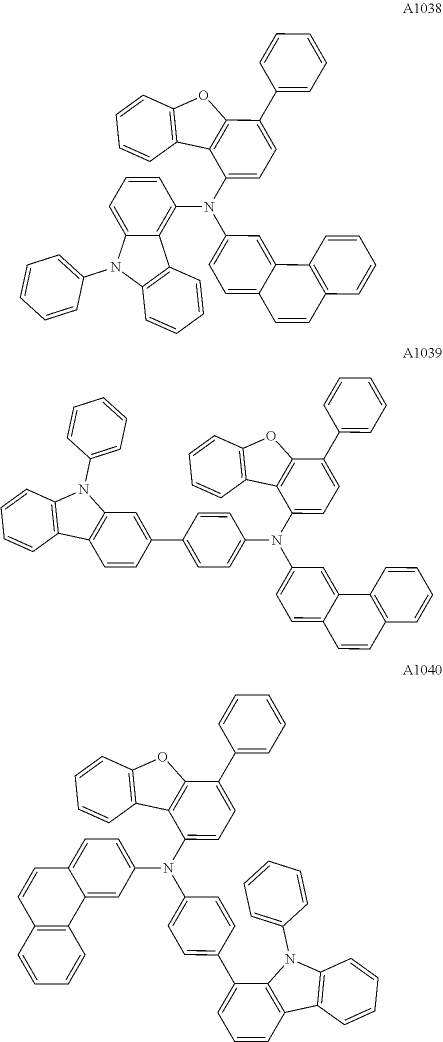 [SMILES: c1ccc(-c2ccc(N(c3ccc4ccc5ccccc5c4c3)c3cccc4c3c3ccccc3n4-c3ccccc3)c3c2oc2ccccc23)cc1 ; c1ccc(-c2ccc(N(c3ccc(-c4ccc5c6ccccc6n(-c6ccccc6)c5c4)cc3)c3ccc4ccc5ccccc5c4c3)c3c2oc2ccccc23)cc1 ; c1ccc(-c2ccc(N(c3ccc(-c4cccc5c6ccccc6n(-c6ccccc6)c45)cc3)c3ccc4ccc5ccccc5c4c3)c3c2oc2ccccc23)cc1] 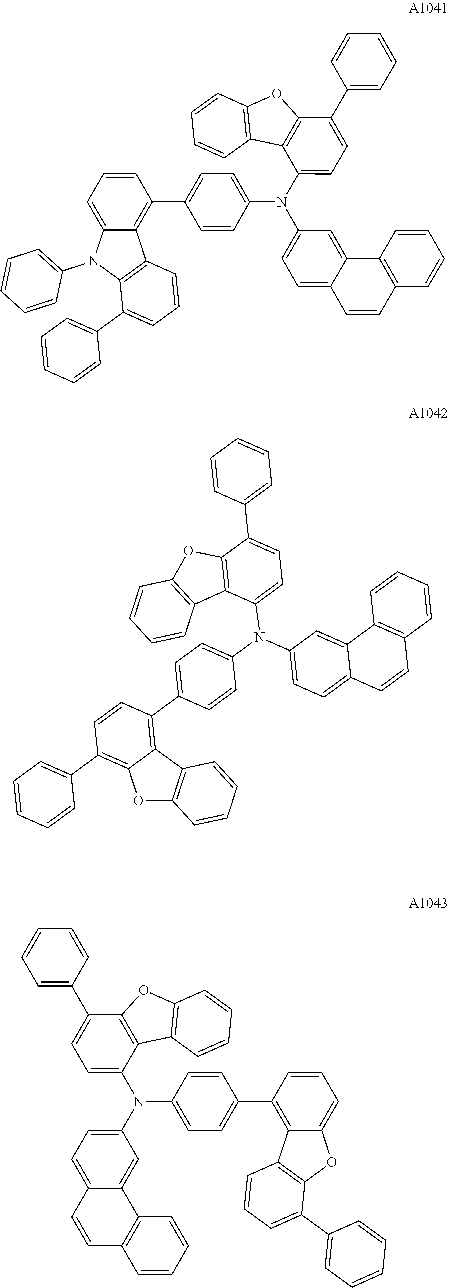 [SMILES: c1ccc(-c2ccc(N(c3ccc(-c4cccc5c4c4cccc(-c6ccccc6)c4n5-c4ccccc4)cc3)c3ccc4ccc5ccccc5c4c3)c3c2oc2ccccc23)cc1 ; c1ccc(-c2ccc(-c3ccc(N(c4ccc5ccc6ccccc6c5c4)c4ccc(-c5ccccc5)c5oc6ccccc6c45)cc3)c3c2oc2ccccc23)cc1 ; c1ccc(-c2cccc3c2oc2cccc(-c4ccc(N(c5ccc6ccc7ccccc7c6c5)c5ccc(-c6ccccc6)c6oc7ccccc7c56)cc4)c23)cc1]
wherein in Compound Group 1,

11. An amine compound represented by Formula 1: 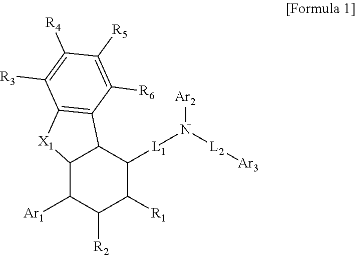
wherein in Formula 1,
X₁ is O or S,
L₁ and L₂ are each independently a direct linkage, a substituted or unsubstituted arylene group having 6 to 15 ring-forming carbon atoms, or a substituted or unsubstituted heteroarylene group having 2 to 15 ring-forming carbon atoms,
Ar₁ is a substituted or unsubstituted aryl group having 6 to 15 ring-forming carbon atoms or a substituted or unsubstituted heteroaryl group having 2 to 15 ring-forming carbon atoms,
Ar₂ is a group represented by Formula 2,
Ar₃ is a substituted or unsubstituted aryl group having 6 to 30 ring-forming carbon atoms or a substituted or unsubstituted heteroaryl group having 2 to 30 ring-forming carbon atoms,
R₁ to R₆ are each independently a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms; 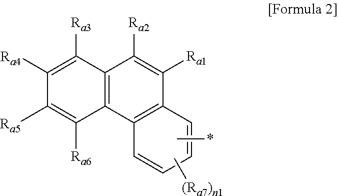
wherein in Formula 2,
n1 is an integer from 0 to 3,
Rₐ₁ to Rₐ₇ are each independently a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms, and [symbol]
represents a bond to a neighboring atom.

12. The amine compound of claim 11, wherein the amine compound represented by Formula 1 is represented by one of Formulas 1-B1 to 1-B4: 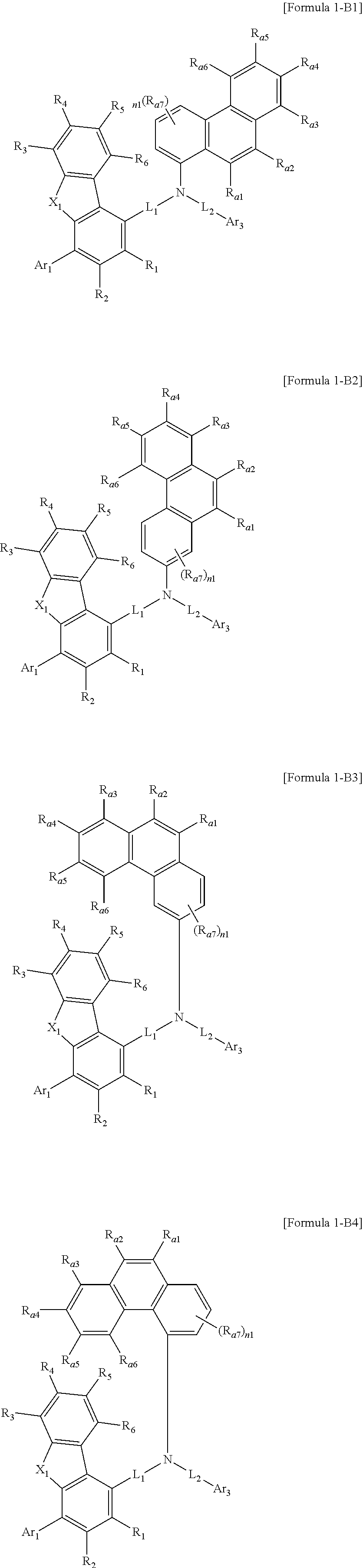
wherein in Formulas 1-B1 to 1-B4,
L₁, L₂, Aᵣ₁, Ar₂, Ar₃, R₁ to R₆, Rai to Rₐ7, and n1 are the same as defined in Formulas 1 and 2.

13. The amine compound of claim 12, wherein in Formulas 1-B1 to 1-B4, Rₐ₁ to Rₐ₇ are each independently a hydrogen atom or a deuterium atom.

14. The amine compound of claim 11, wherein in Formula 1, An is a group represented by one of Formulas AR-1 to AR-4: 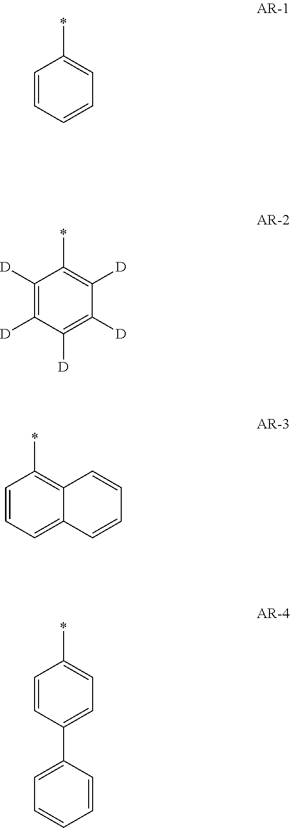 [SMILES: *c1ccccc1 ; *c1c([2H])c([2H])c([2H])c([2H])c1[2H] ; *c1cccc2ccccc12 ; *c1ccc(-c2ccccc2)cc1]
wherein in Formula AR-2,
D is a deuterium atom.

15. The amine compound of claim 11, wherein in Formula 1, Li is a direct linkage, a substituted or unsubstituted phenylene group, a substituted or unsubstituted naphthylene group, a substituted or unsubstituted divalent dibenzofuran group, or a substituted or unsubstituted divalent dibenzothiophene group.

16. The amine compound of claim 11, wherein in Formula 1, Li is a direct linkage or a group represented by one of Formulas L1-1 to L1-5: 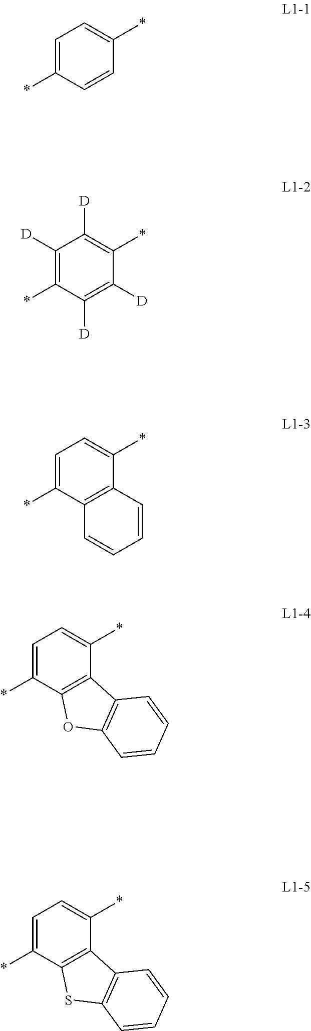 [SMILES: *c1ccc(*)cc1 ; *c1c([2H])c([2H])c(*)c([2H])c1[2H] ; *c1ccc(*)c2ccccc12 ; *c1ccc(*)c2c1oc1ccccc12 ; *c1ccc(*)c2c1sc1ccccc12]
wherein in Formula L1-2,
D is a deuterium atom.

17. The amine compound of claim 11, wherein in Formula 1,
L₂ is a direct linkage, a substituted or unsubstituted phenylene group, a substituted or unsubstituted biphenylene group, a substituted or unsubstituted naphthylene group, a substituted or unsubstituted divalent carbazole group, a substituted or unsubstituted divalent dibenzofuran group, or a substituted or unsubstituted divalent dibenzothiophene group, and
Ar₃ is a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, a substituted or unsubstituted carbazole group, a substituted or unsubstituted dibenzofuran group, or a substituted or unsubstituted dibenzothiophene group.

18. The amine compound of claim 11, wherein in Formula 1, *-L₂-Ar₃ is a moiety represented by one of Formulas LA-1 to LA-79: 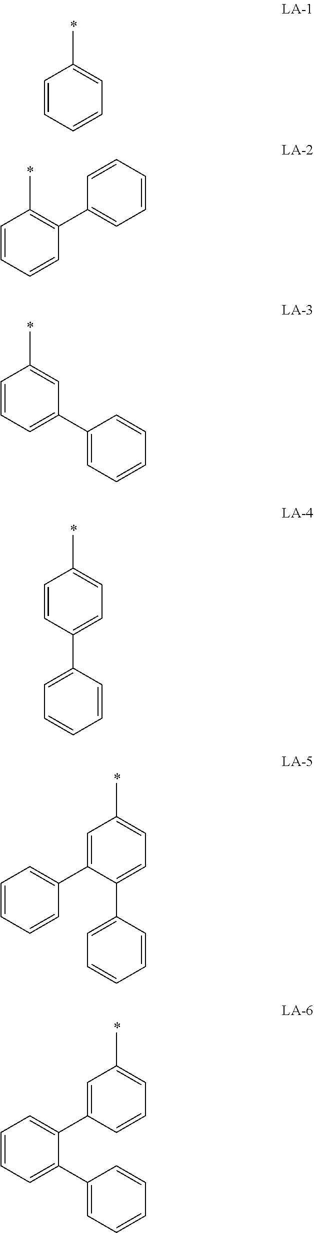 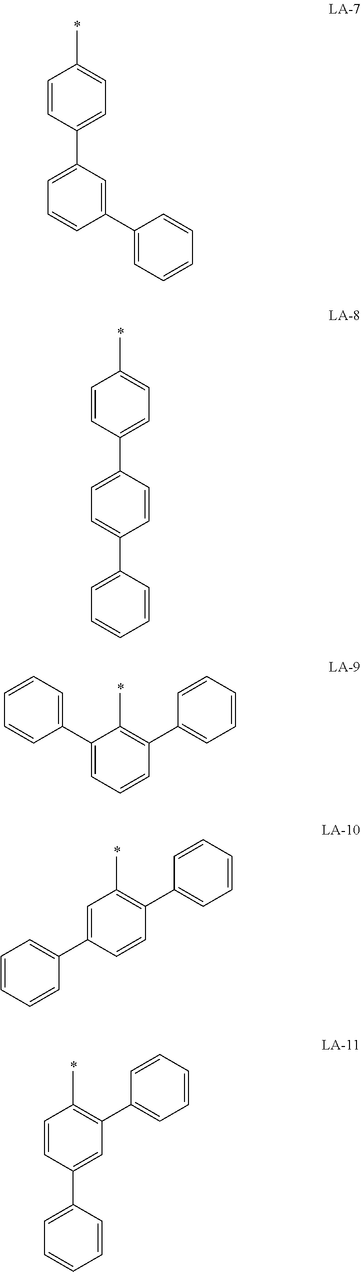 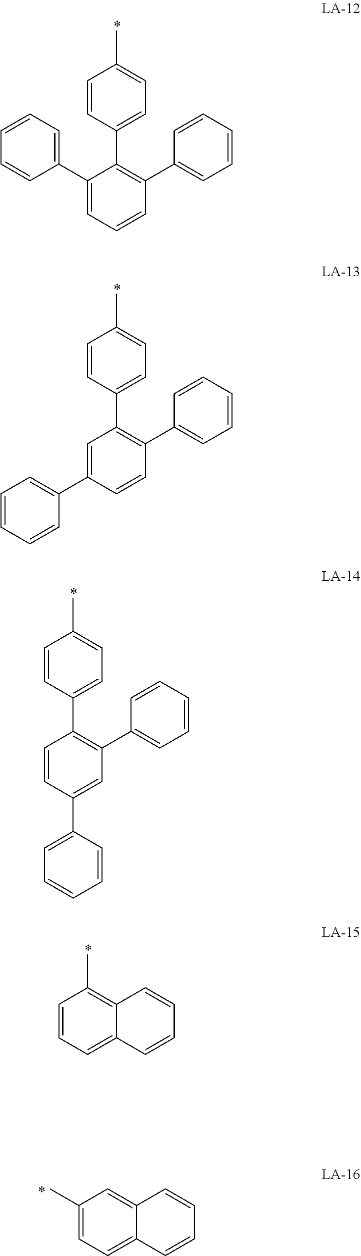 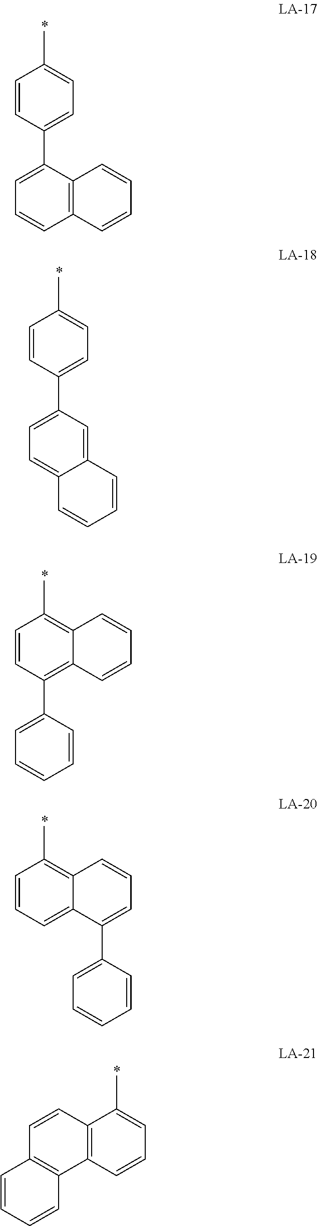 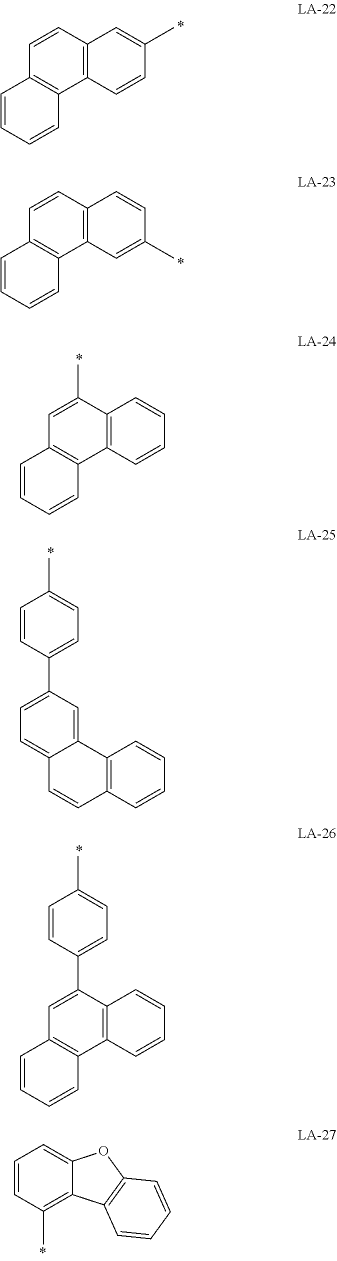 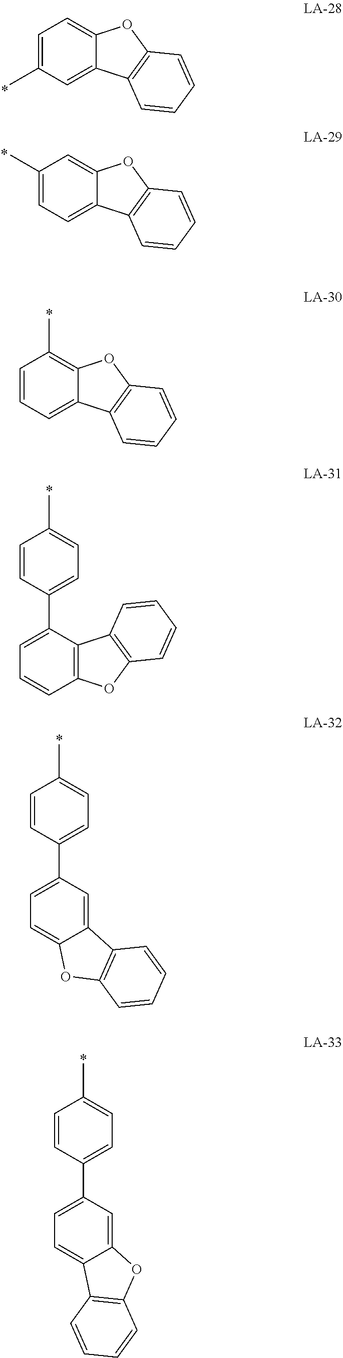 [SMILES: Cc1ccc2oc3ccccc3c2c1 ; Cc1ccc2c(c1)oc1ccccc12 ; Cc1cccc2c1oc1ccccc12 ; Cc1ccc(-c2cccc3oc4ccccc4c23)cc1 ; Cc1ccc(-c2ccc3oc4ccccc4c3c2)cc1 ; Cc1ccc(-c2ccc3c(c2)oc2ccccc23)cc1] 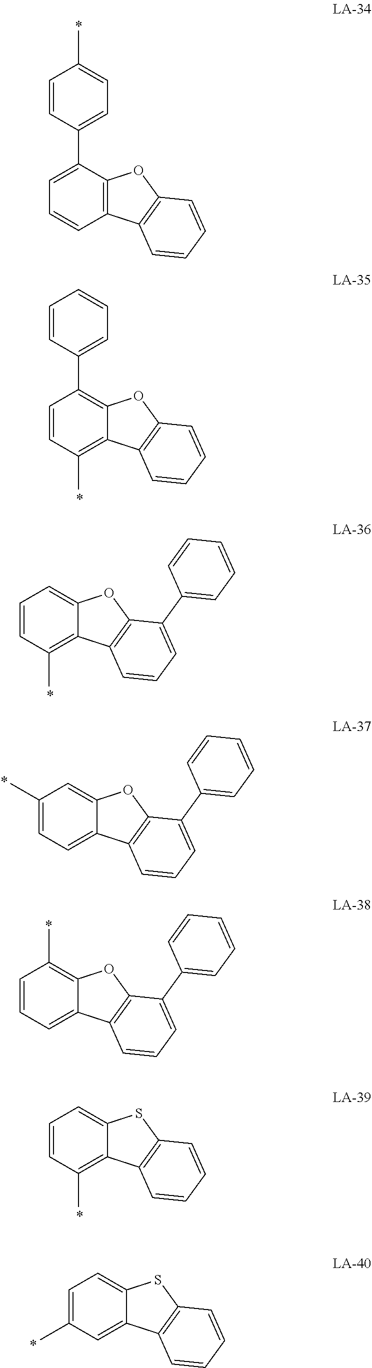 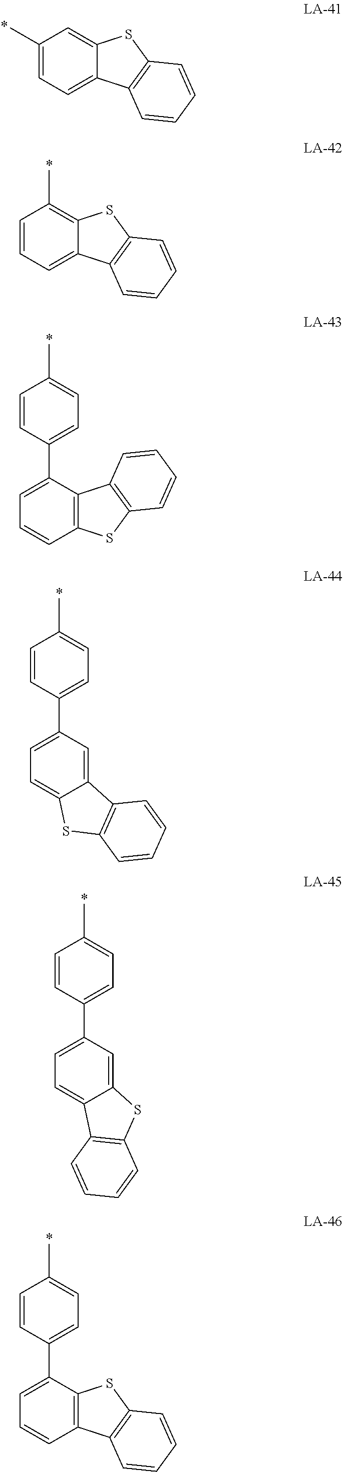 [SMILES: Cc1ccc2c(c1)sc1ccccc12 ; Cc1cccc2c1sc1ccccc12 ; Cc1ccc(-c2cccc3sc4ccccc4c23)cc1 ; Cc1ccc(-c2ccc3sc4ccccc4c3c2)cc1 ; Cc1ccc(-c2ccc3c(c2)sc2ccccc23)cc1 ; Cc1ccc(-c2cccc3c2sc2ccccc23)cc1] 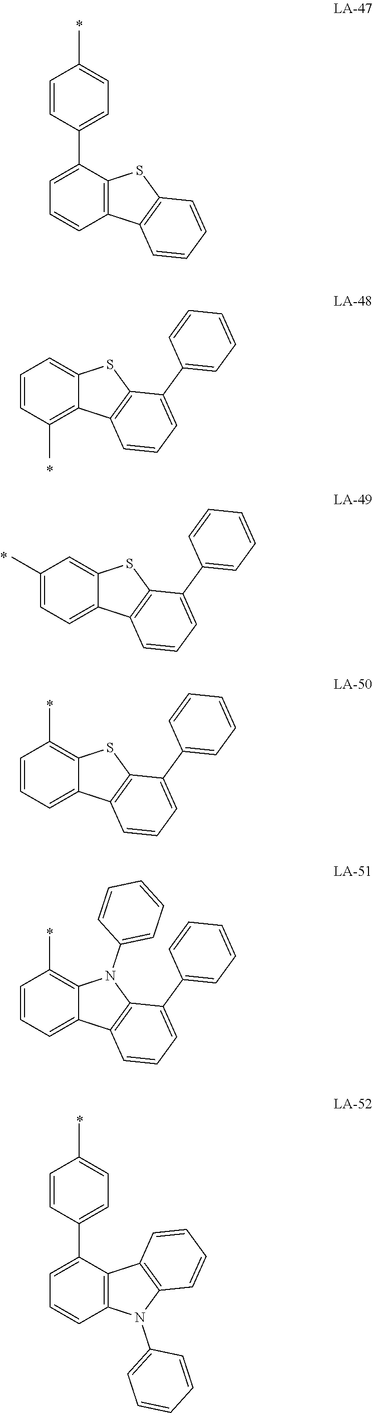 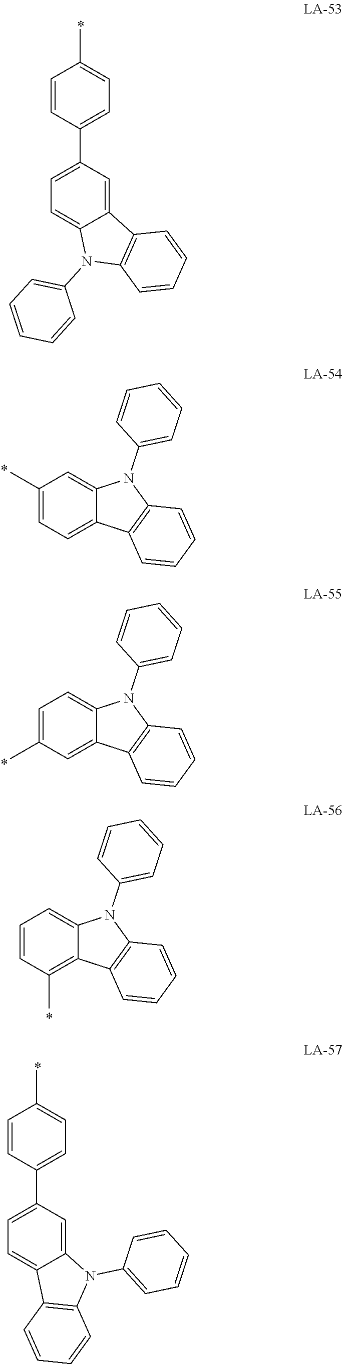 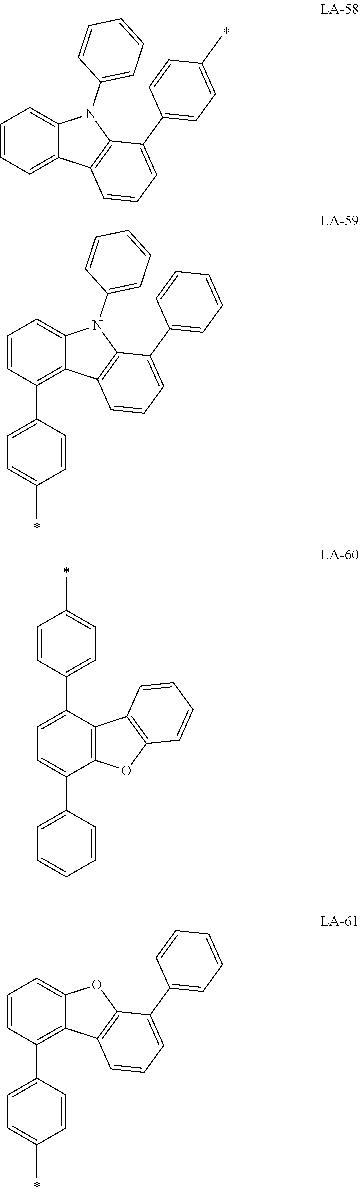 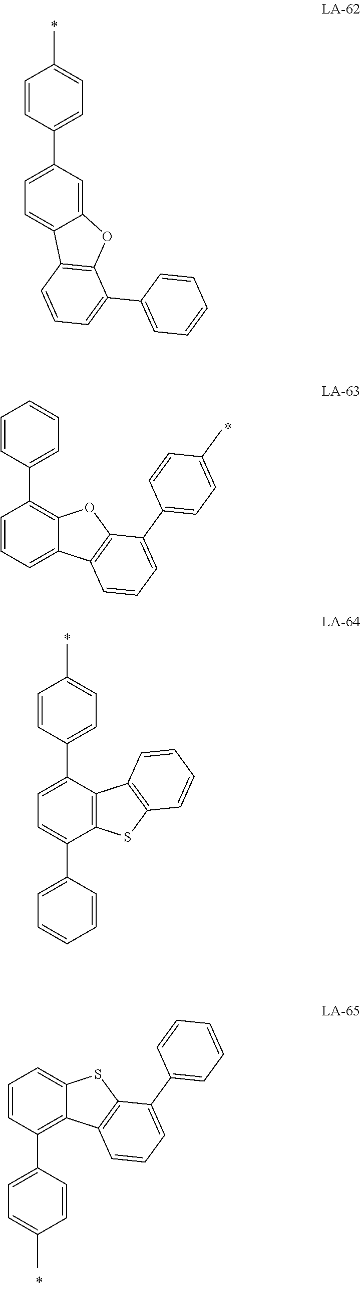 [SMILES: Cc1ccc(-c2ccc3c(c2)oc2c(-c4ccccc4)cccc23)cc1 ; Cc1ccc(-c2cccc3c2oc2c(-c4ccccc4)cccc23)cc1 ; Cc1ccc(-c2ccc(-c3ccccc3)c3sc4ccccc4c23)cc1 ; Cc1ccc(-c2cccc3sc4c(-c5ccccc5)cccc4c23)cc1] 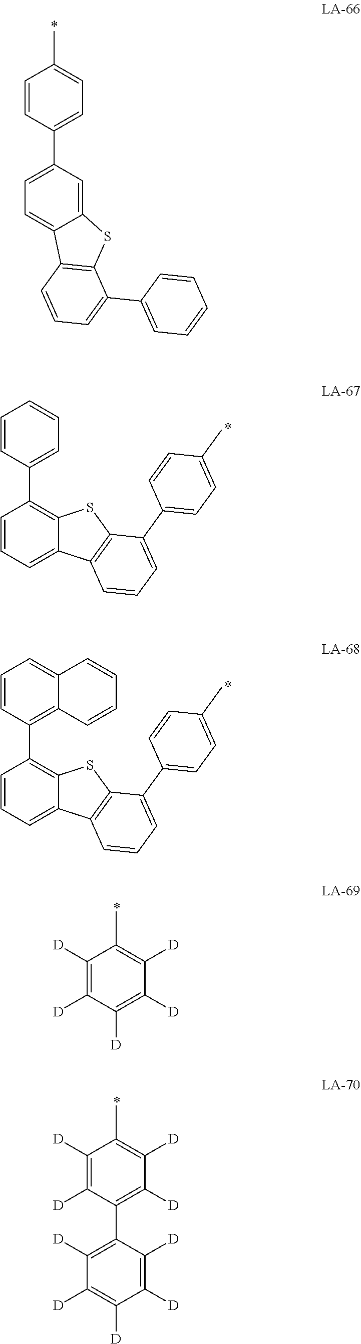 [SMILES: Cc1ccc(-c2ccc3c(c2)sc2c(-c4ccccc4)cccc23)cc1 ; Cc1ccc(-c2cccc3c2sc2c(-c4ccccc4)cccc23)cc1 ; Cc1ccc(-c2cccc3c2sc2c(-c4cccc5ccccc45)cccc23)cc1 ; [2H]c1c([2H])c([2H])c(C)c([2H])c1[2H] ; [2H]c1c([2H])c([2H])c(-c2c([2H])c([2H])c(C)c([2H])c2[2H])c([2H])c1[2H]] 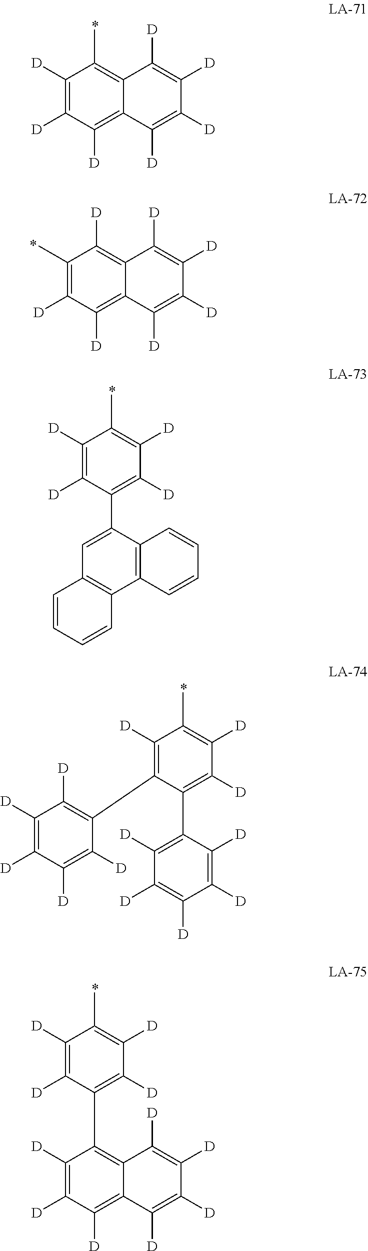 [SMILES: [2H]c1c([2H])c([2H])c2c(C)c([2H])c([2H])c([2H])c2c1[2H] ; [2H]c1c([2H])c([2H])c2c([2H])c(C)c([2H])c([2H])c2c1[2H] ; [2H]c1c([2H])c(-c2cc3ccccc3c3ccccc23)c([2H])c([2H])c1C ; [2H]c1c([2H])c([2H])c(-c2c([2H])c([2H])c(C)c([2H])c2-c2c([2H])c([2H])c([2H])c([2H])c2[2H])c([2H])c1[2H] ; [2H]c1c([2H])c(-c2c([2H])c([2H])c([2H])c3c([2H])c([2H])c([2H])c([2H])c23)c([2H])c([2H])c1C] 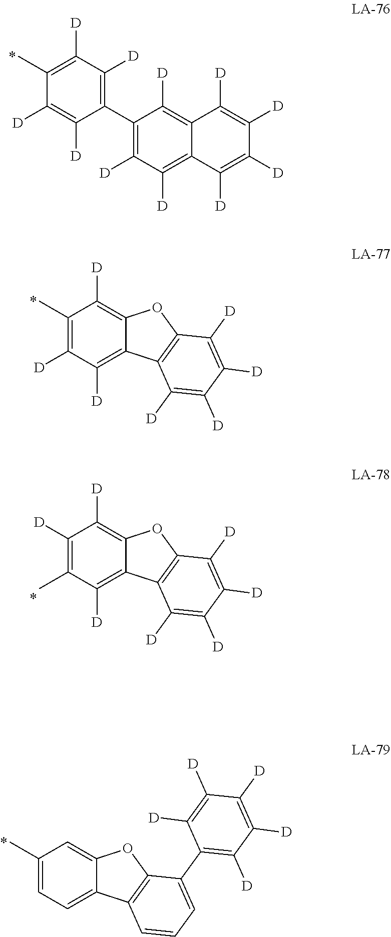 [SMILES: [2H]c1c([2H])c(-c2c([2H])c([2H])c3c([2H])c([2H])c([2H])c([2H])c3c2[2H])c([2H])c([2H])c1C ; [2H]c1c([2H])c([2H])c2c(oc3c([2H])c(C)c([2H])c([2H])c32)c1[2H] ; [2H]c1c([2H])c([2H])c2c(oc3c([2H])c([2H])c(C)c([2H])c32)c1[2H] ; [2H]c1c([2H])c([2H])c(-c2cccc3c2oc2cc(C)ccc23)c([2H])c1[2H]]
wherein in Formulas LA-69 to LA-79,
D is a deuterium atom.

19. The amine compound of claim 11, wherein the amine compound represented by Formula 1 is selected from Compound Group 1: 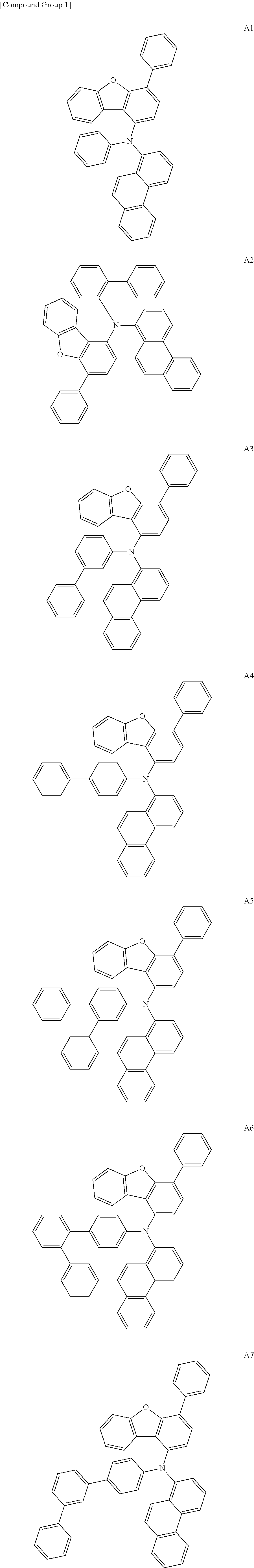 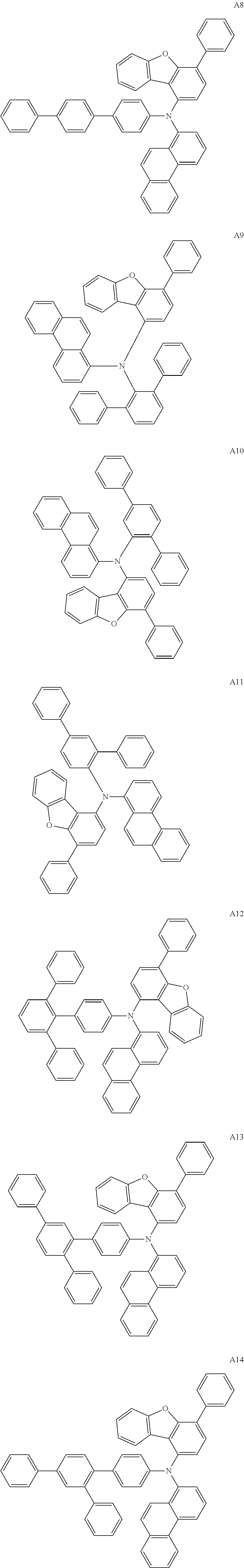 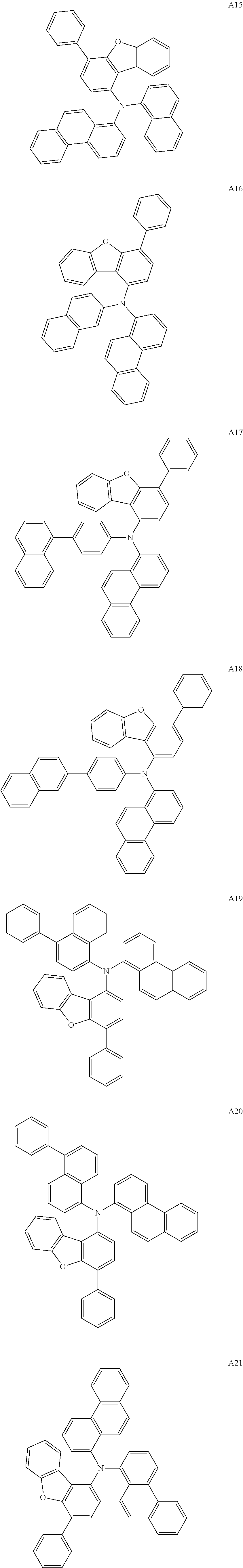 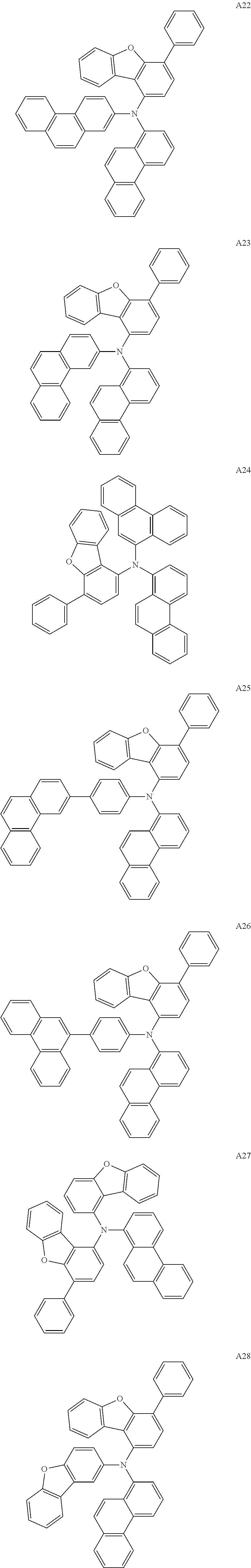 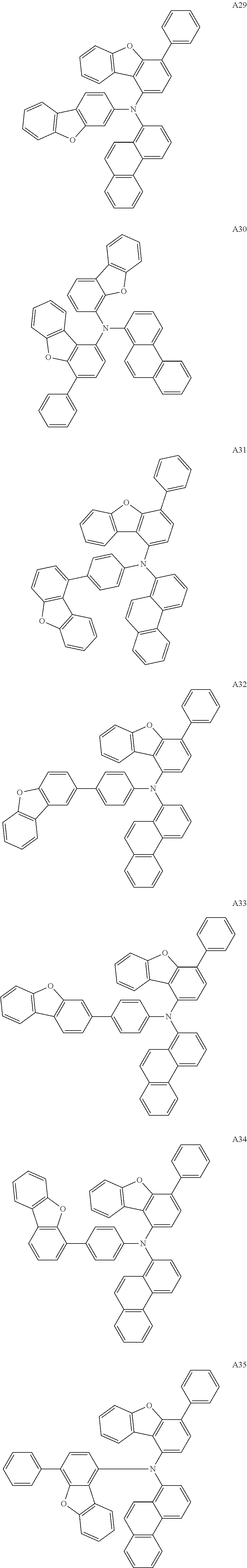 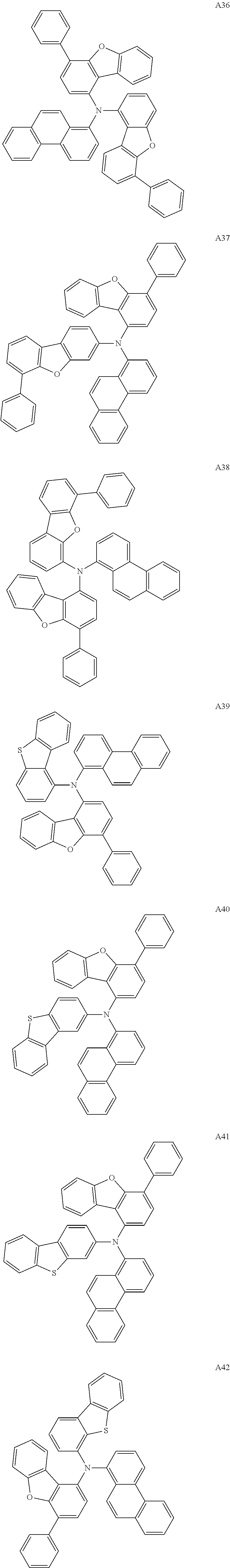 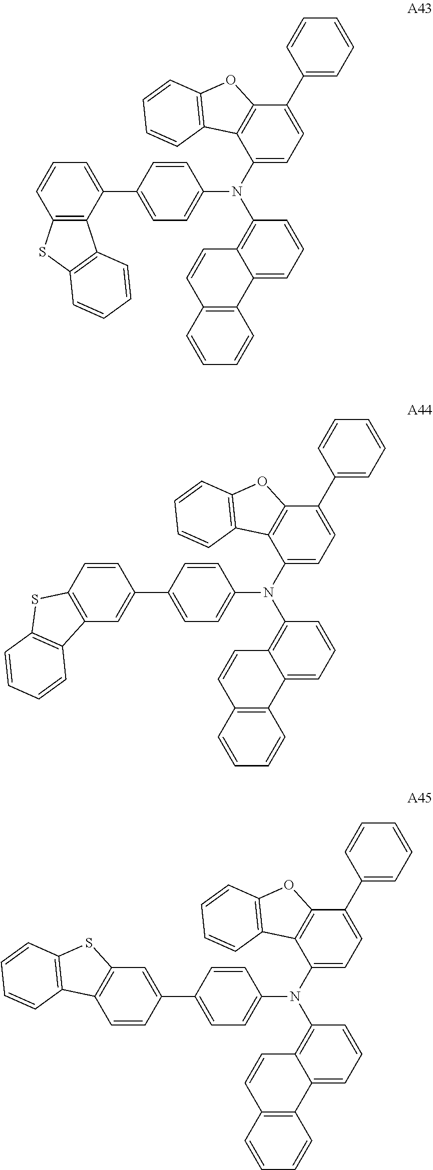 [SMILES: c1ccc(-c2ccc(N(c3ccc(-c4cccc5sc6ccccc6c45)cc3)c3cccc4c3ccc3ccccc34)c3c2oc2ccccc23)cc1 ; c1ccc(-c2ccc(N(c3ccc(-c4ccc5sc6ccccc6c5c4)cc3)c3cccc4c3ccc3ccccc34)c3c2oc2ccccc23)cc1 ; c1ccc(-c2ccc(N(c3ccc(-c4ccc5c(c4)sc4ccccc45)cc3)c3cccc4c3ccc3ccccc34)c3c2oc2ccccc23)cc1] 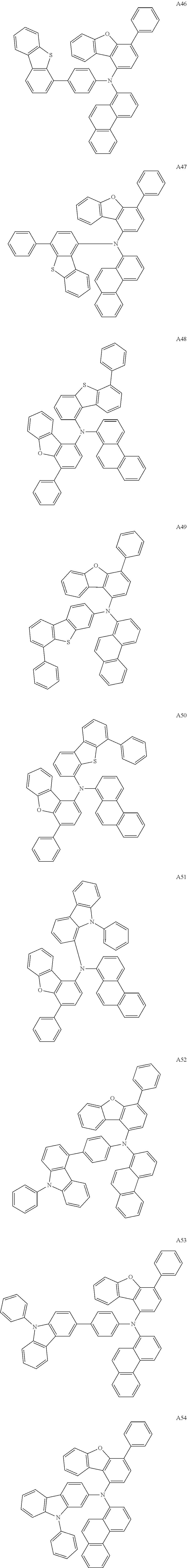 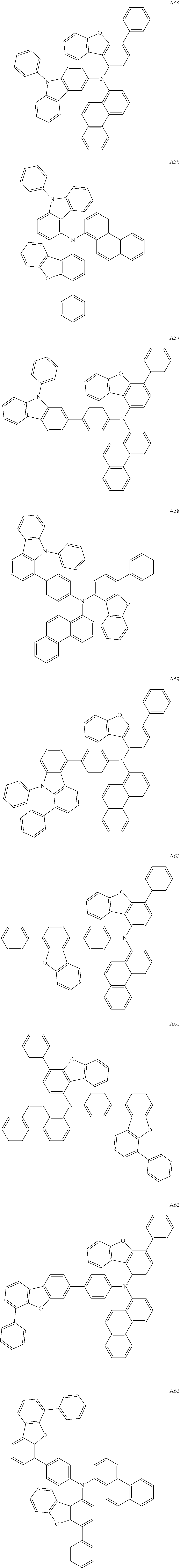 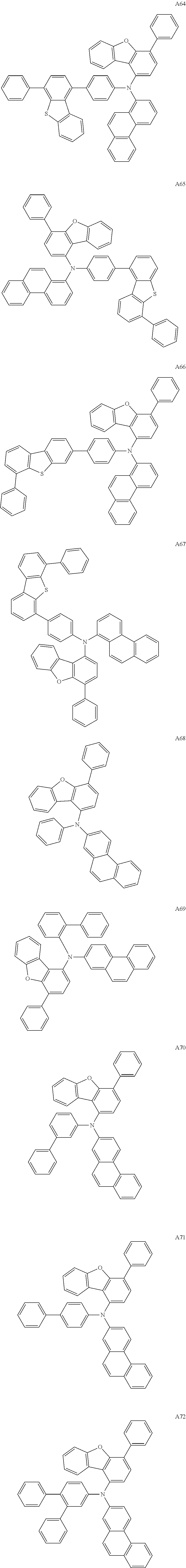 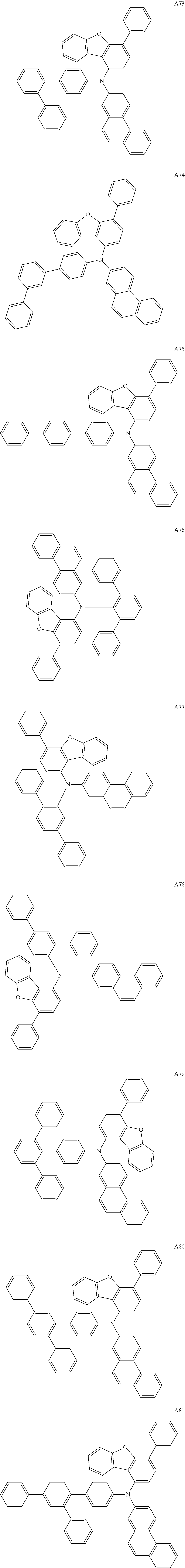 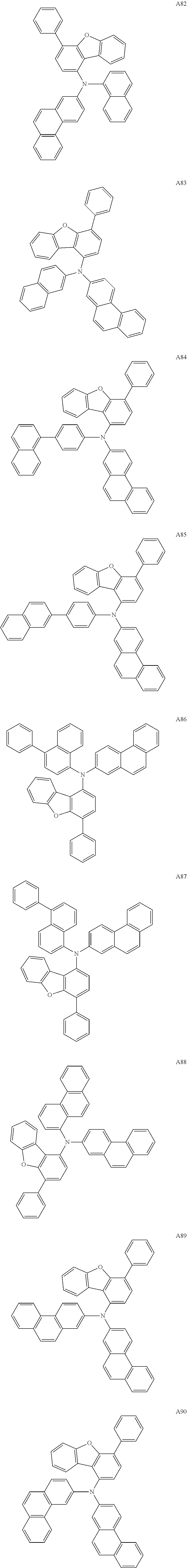 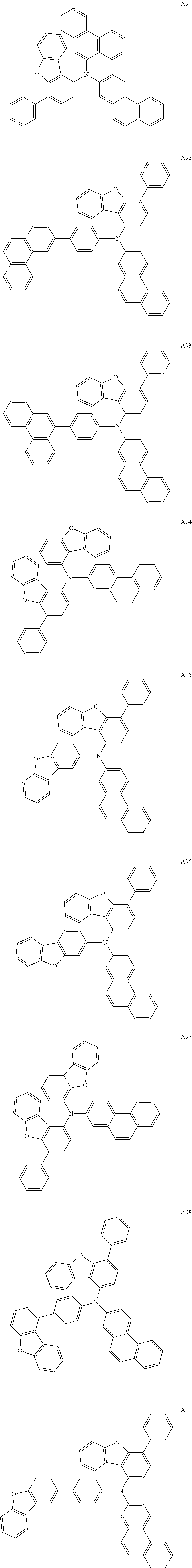 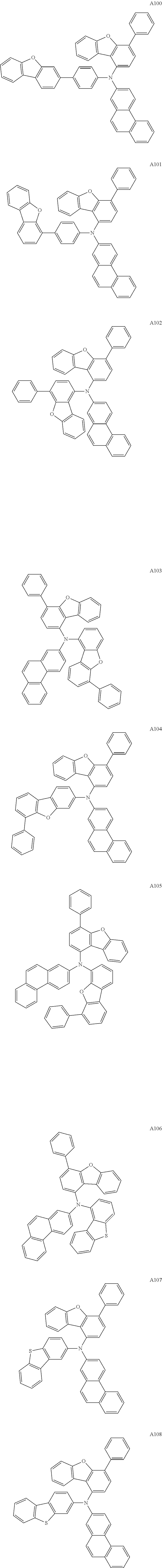 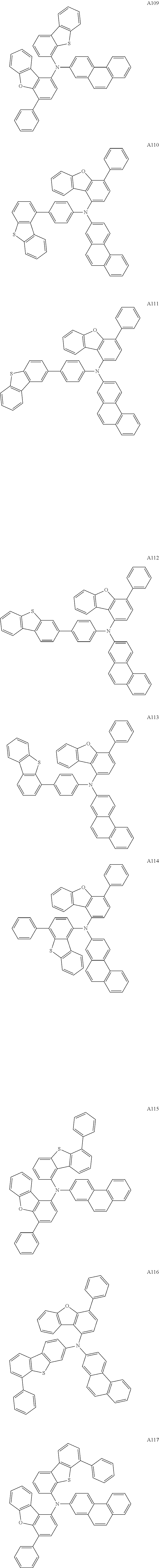 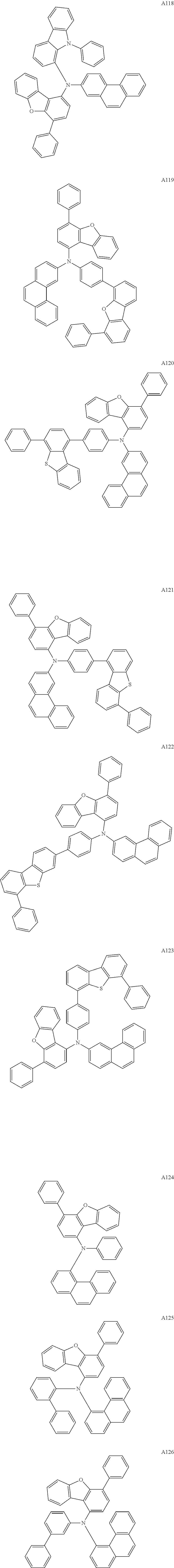 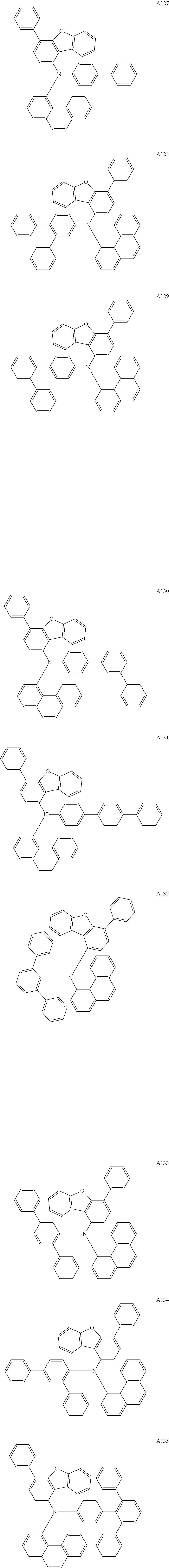 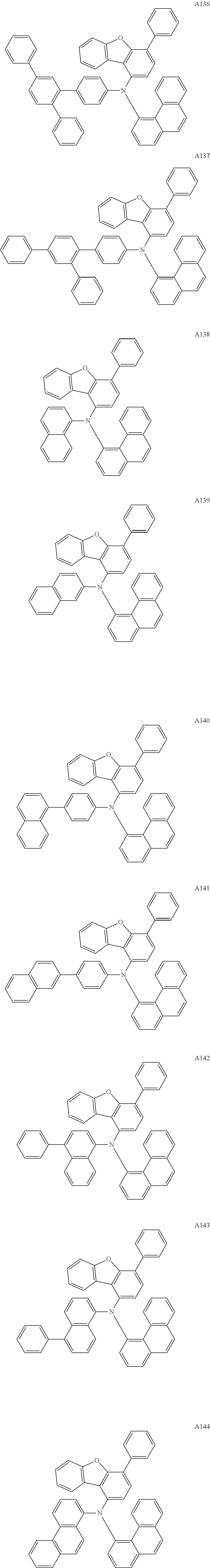 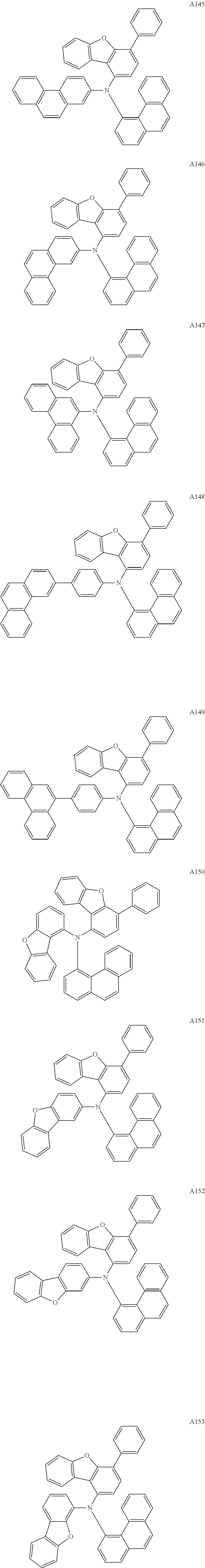 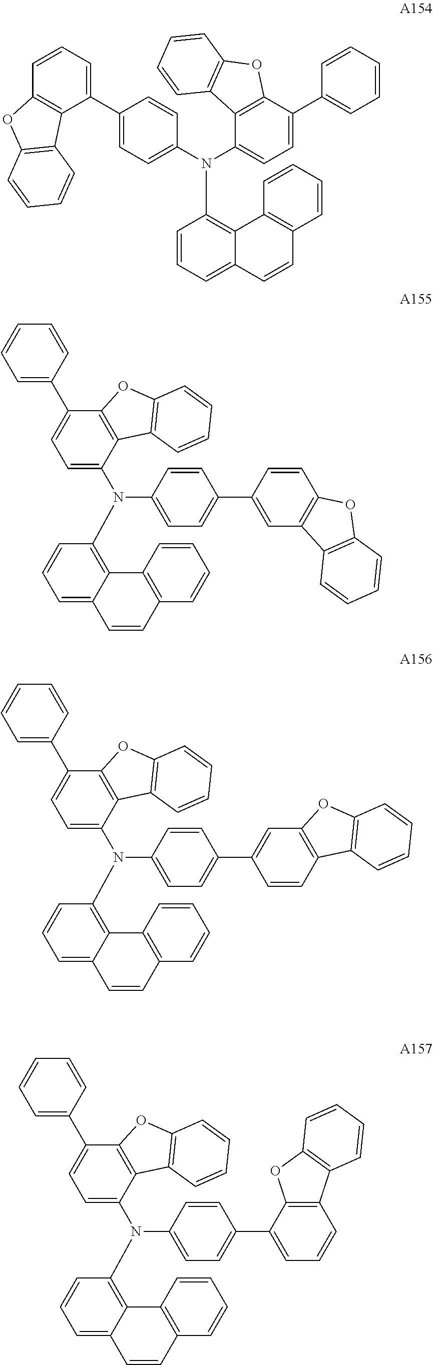 [SMILES: c1ccc(-c2ccc(N(c3ccc(-c4cccc5oc6ccccc6c45)cc3)c3cccc4ccc5ccccc5c34)c3c2oc2ccccc23)cc1 ; c1ccc(-c2ccc(N(c3ccc(-c4ccc5oc6ccccc6c5c4)cc3)c3cccc4ccc5ccccc5c34)c3c2oc2ccccc23)cc1 ; c1ccc(-c2ccc(N(c3ccc(-c4ccc5c(c4)oc4ccccc45)cc3)c3cccc4ccc5ccccc5c34)c3c2oc2ccccc23)cc1 ; c1ccc(-c2ccc(N(c3ccc(-c4cccc5c4oc4ccccc45)cc3)c3cccc4ccc5ccccc5c34)c3c2oc2ccccc23)cc1] 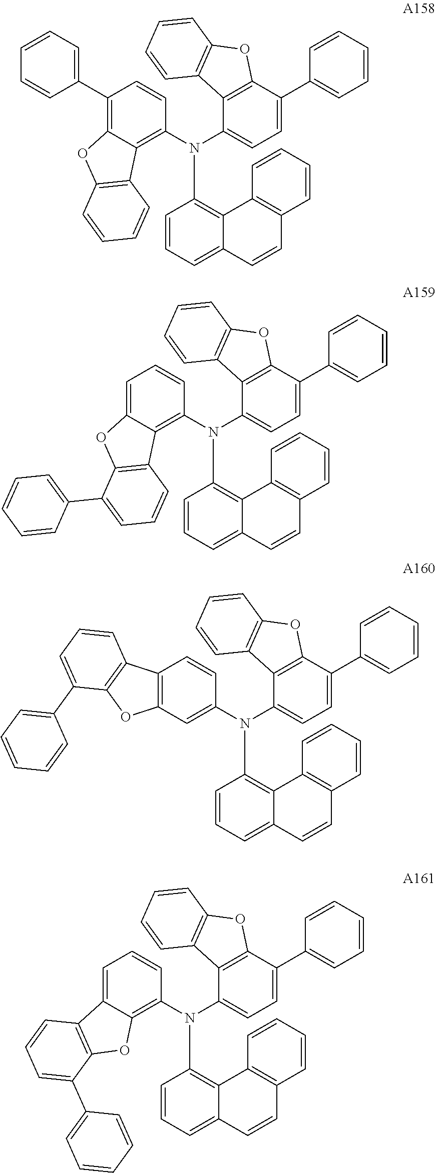 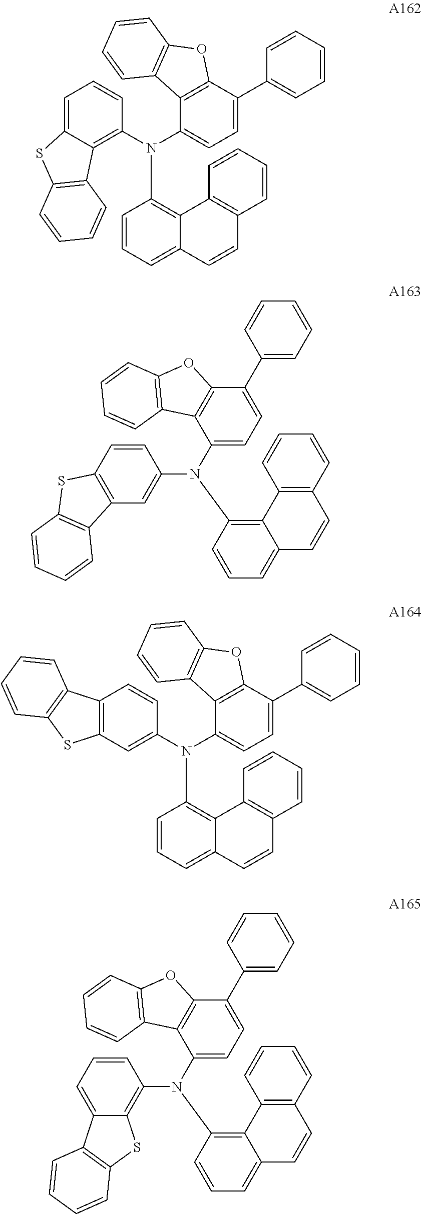 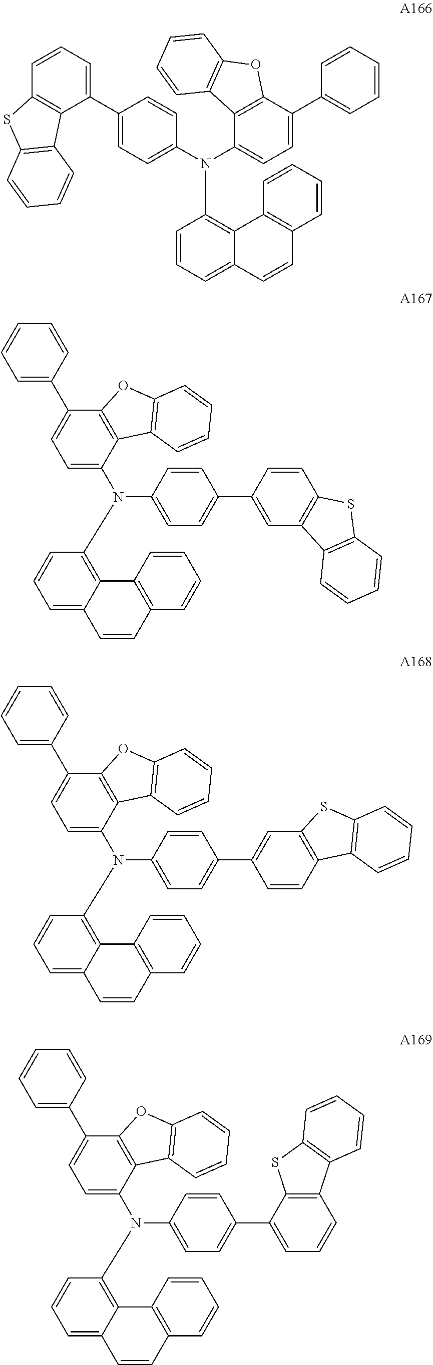 [SMILES: c1ccc(-c2ccc(N(c3ccc(-c4cccc5sc6ccccc6c45)cc3)c3cccc4ccc5ccccc5c34)c3c2oc2ccccc23)cc1 ; c1ccc(-c2ccc(N(c3ccc(-c4ccc5sc6ccccc6c5c4)cc3)c3cccc4ccc5ccccc5c34)c3c2oc2ccccc23)cc1 ; c1ccc(-c2ccc(N(c3ccc(-c4ccc5c(c4)sc4ccccc45)cc3)c3cccc4ccc5ccccc5c34)c3c2oc2ccccc23)cc1 ; c1ccc(-c2ccc(N(c3ccc(-c4cccc5c4sc4ccccc45)cc3)c3cccc4ccc5ccccc5c34)c3c2oc2ccccc23)cc1] 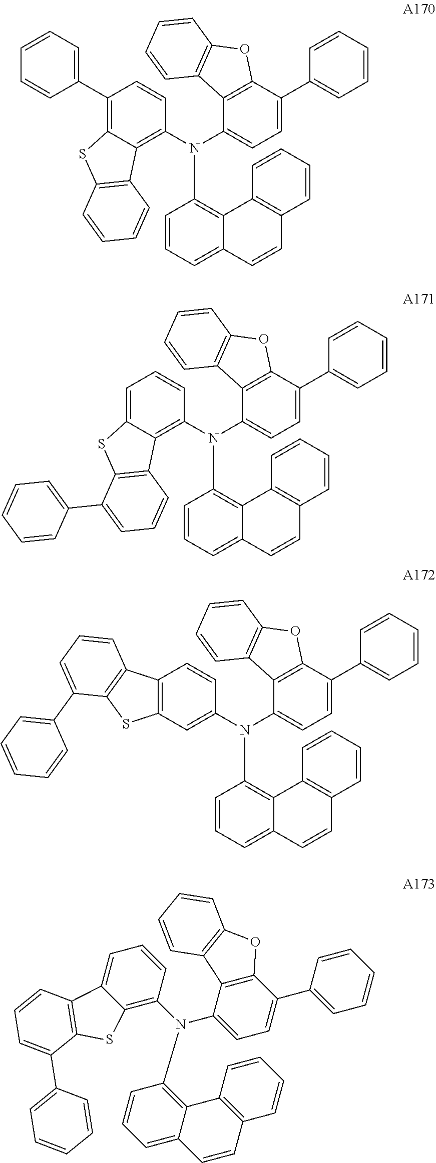 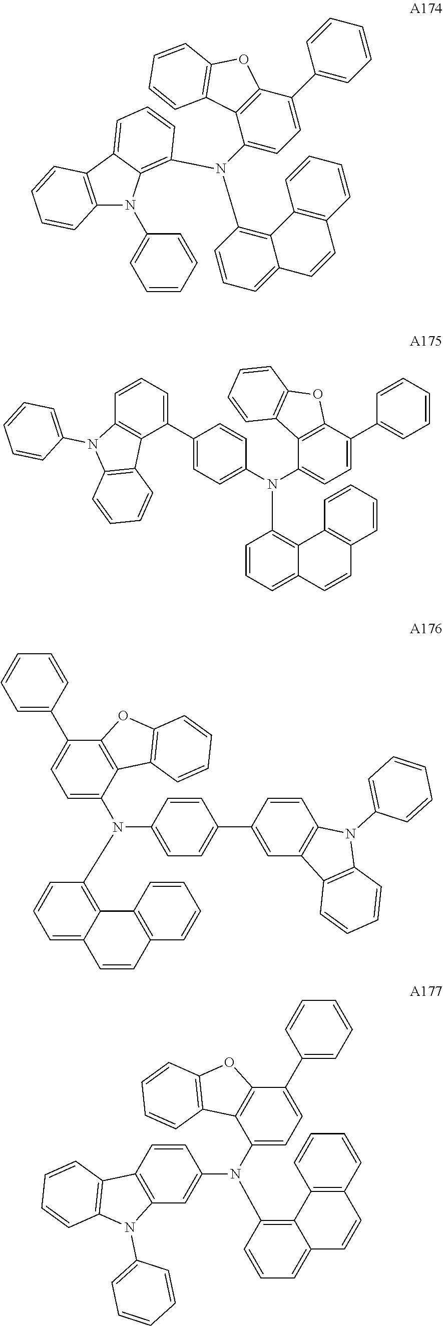 [SMILES: c1ccc(-c2ccc(N(c3cccc4ccc5ccccc5c34)c3cccc4c5ccccc5n(-c5ccccc5)c34)c3c2oc2ccccc23)cc1 ; c1ccc(-c2ccc(N(c3ccc(-c4cccc5c4c4ccccc4n5-c4ccccc4)cc3)c3cccc4ccc5ccccc5c34)c3c2oc2ccccc23)cc1 ; c1ccc(-c2ccc(N(c3ccc(-c4ccc5c(c4)c4ccccc4n5-c4ccccc4)cc3)c3cccc4ccc5ccccc5c34)c3c2oc2ccccc23)cc1 ; c1ccc(-c2ccc(N(c3ccc4c5ccccc5n(-c5ccccc5)c4c3)c3cccc4ccc5ccccc5c34)c3c2oc2ccccc23)cc1] 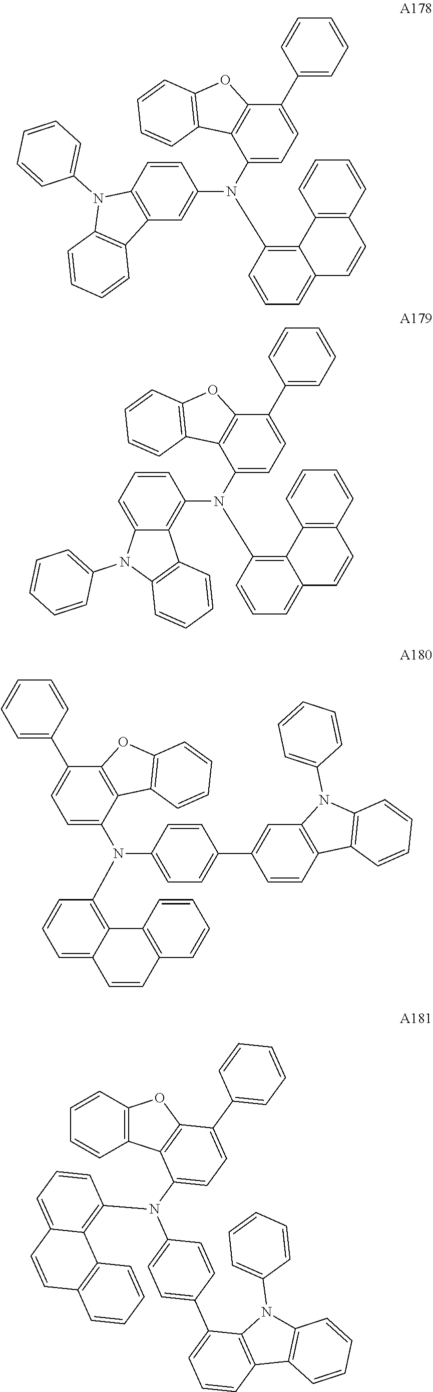 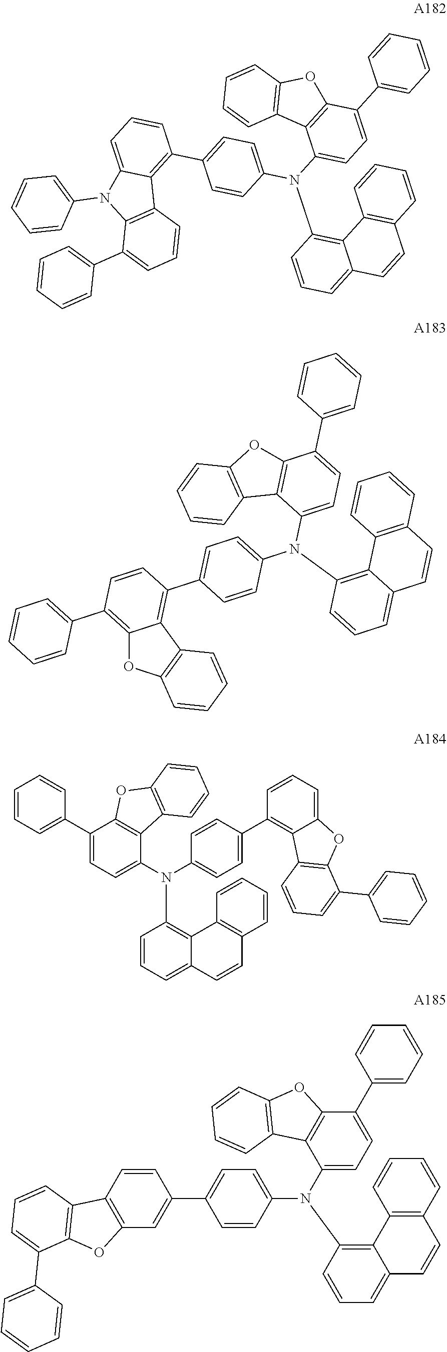 [SMILES: c1ccc(-c2ccc(N(c3ccc(-c4cccc5c4c4cccc(-c6ccccc6)c4n5-c4ccccc4)cc3)c3cccc4ccc5ccccc5c34)c3c2oc2ccccc23)cc1 ; c1ccc(-c2ccc(-c3ccc(N(c4cccc5ccc6ccccc6c45)c4ccc(-c5ccccc5)c5oc6ccccc6c45)cc3)c3c2oc2ccccc23)cc1 ; c1ccc(-c2cccc3c2oc2cccc(-c4ccc(N(c5cccc6ccc7ccccc7c56)c5ccc(-c6ccccc6)c6oc7ccccc7c56)cc4)c23)cc1 ; c1ccc(-c2cccc3c2oc2cc(-c4ccc(N(c5cccc6ccc7ccccc7c56)c5ccc(-c6ccccc6)c6oc7ccccc7c56)cc4)ccc23)cc1] 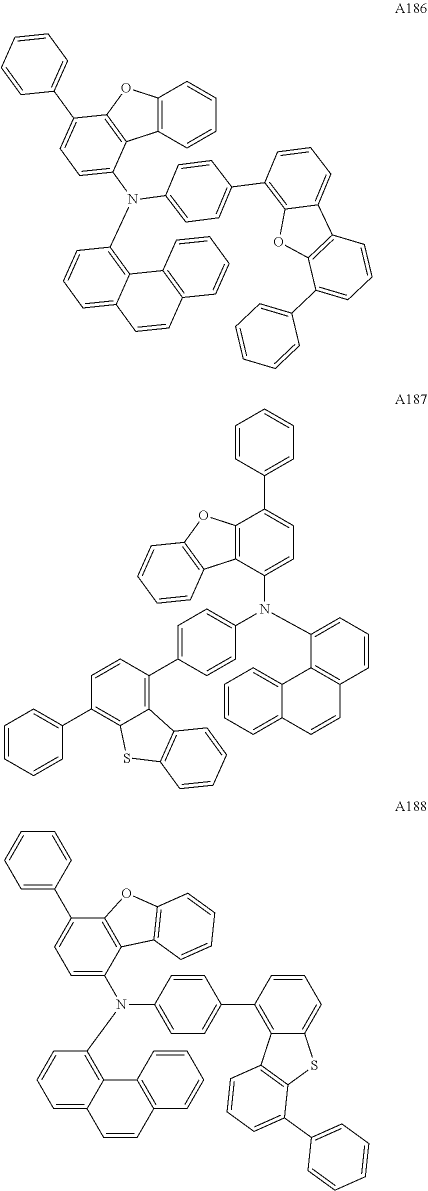 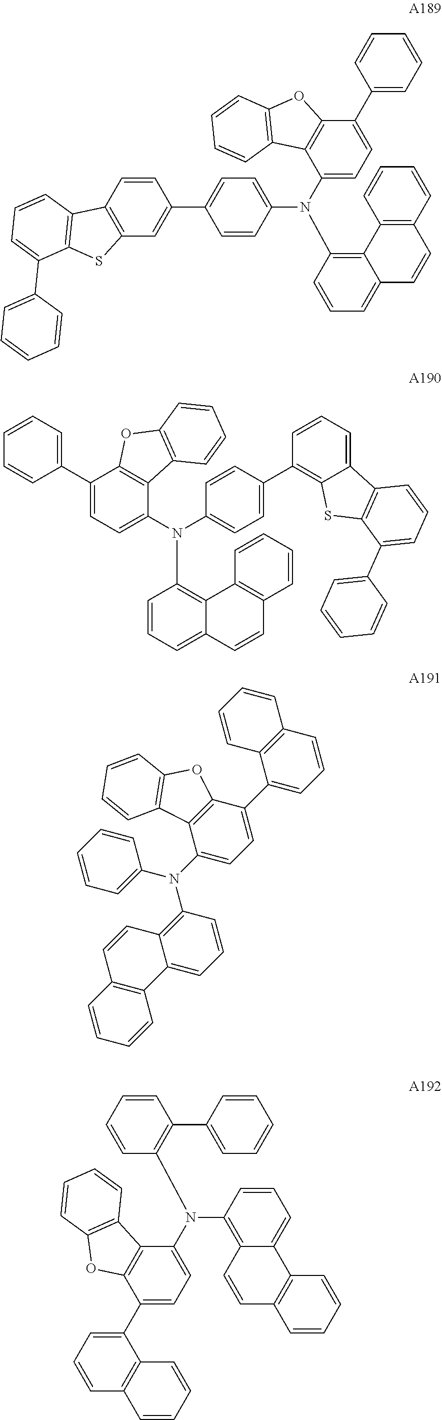 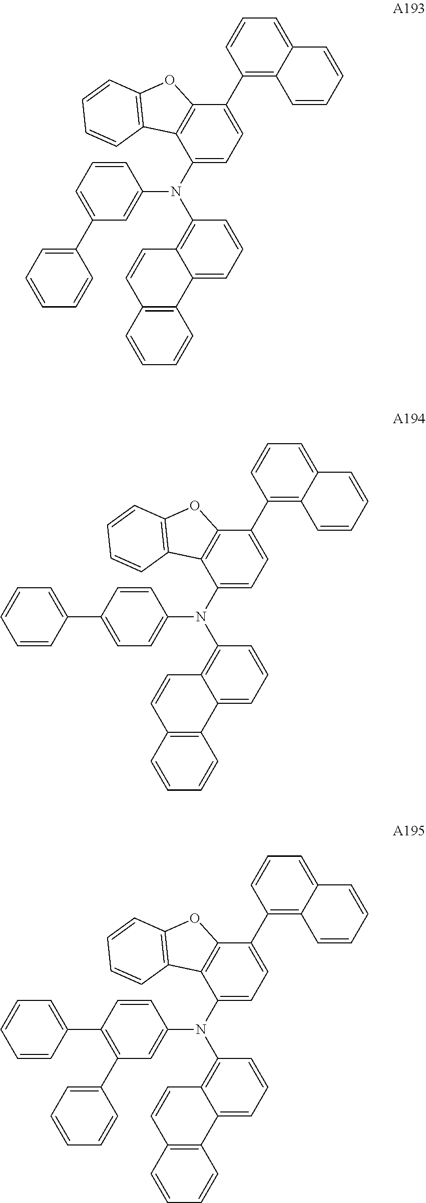 [SMILES: c1ccc(-c2cccc(N(c3cccc4c3ccc3ccccc34)c3ccc(-c4cccc5ccccc45)c4oc5ccccc5c34)c2)cc1 ; c1ccc(-c2ccc(N(c3cccc4c3ccc3ccccc34)c3ccc(-c4cccc5ccccc45)c4oc5ccccc5c34)cc2)cc1 ; c1ccc(-c2ccc(N(c3cccc4c3ccc3ccccc34)c3ccc(-c4cccc5ccccc45)c4oc5ccccc5c34)cc2-c2ccccc2)cc1] 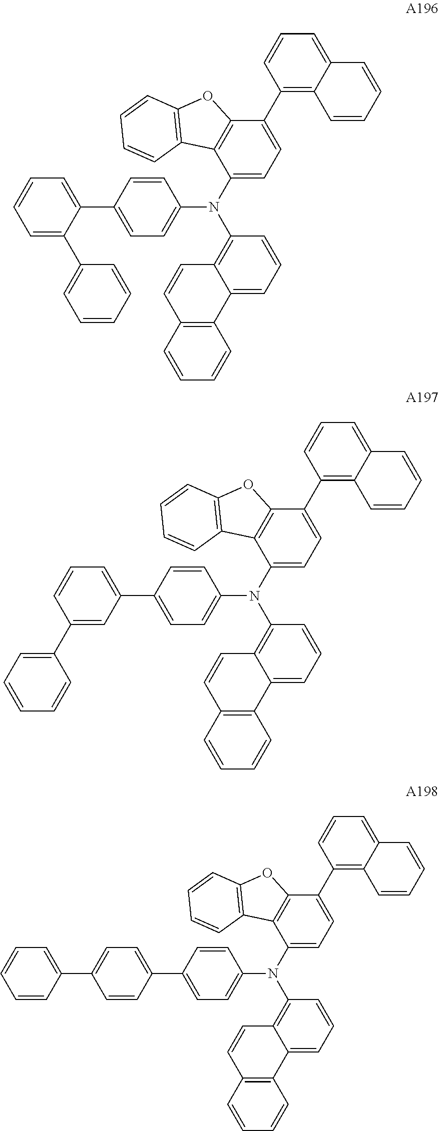 [SMILES: c1ccc(-c2ccccc2-c2ccc(N(c3cccc4c3ccc3ccccc34)c3ccc(-c4cccc5ccccc45)c4oc5ccccc5c34)cc2)cc1 ; c1ccc(-c2cccc(-c3ccc(N(c4cccc5c4ccc4ccccc45)c4ccc(-c5cccc6ccccc56)c5oc6ccccc6c45)cc3)c2)cc1 ; c1ccc(-c2ccc(-c3ccc(N(c4cccc5c4ccc4ccccc45)c4ccc(-c5cccc6ccccc56)c5oc6ccccc6c45)cc3)cc2)cc1] 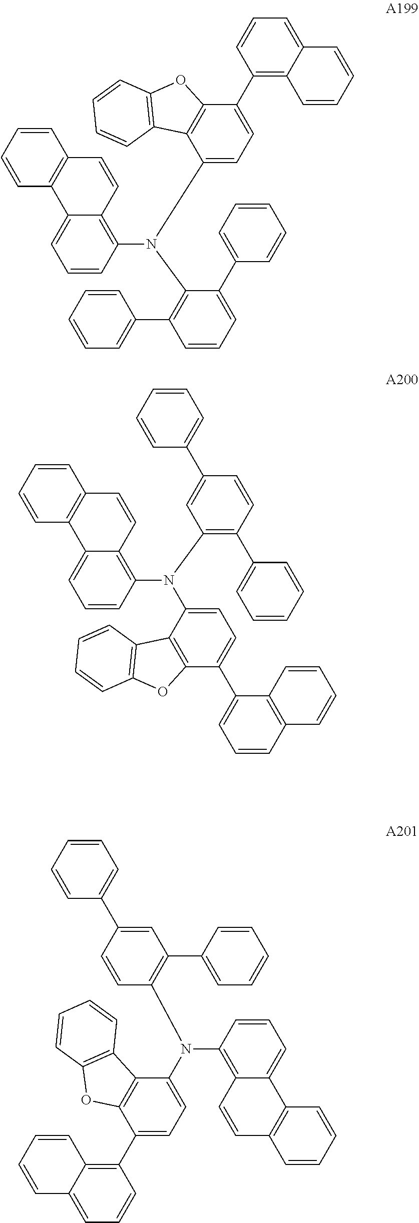 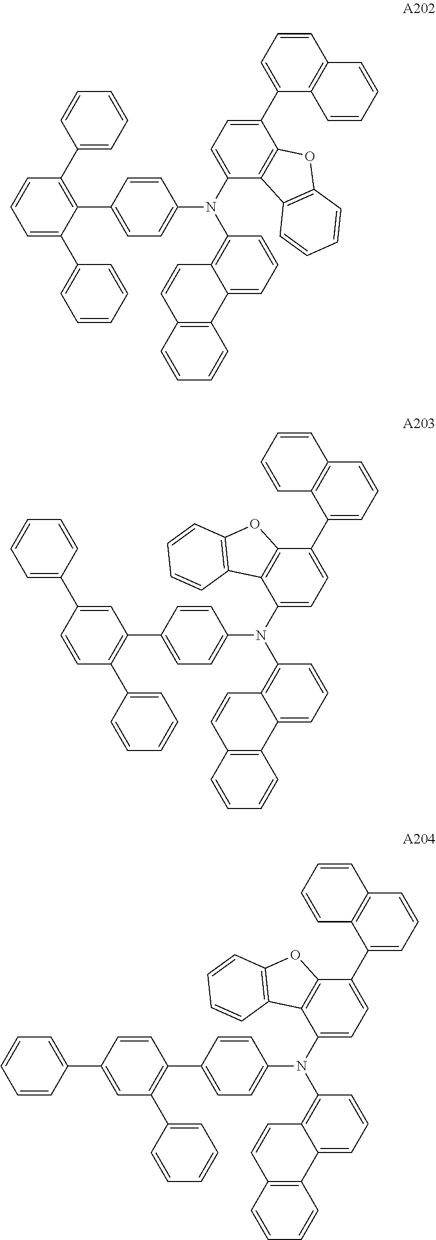 [SMILES: c1ccc(-c2cccc(-c3ccccc3)c2-c2ccc(N(c3cccc4c3ccc3ccccc34)c3ccc(-c4cccc5ccccc45)c4oc5ccccc5c34)cc2)cc1 ; c1ccc(-c2ccc(-c3ccccc3)c(-c3ccc(N(c4cccc5c4ccc4ccccc45)c4ccc(-c5cccc6ccccc56)c5oc6ccccc6c45)cc3)c2)cc1 ; c1ccc(-c2ccc(-c3ccc(N(c4cccc5c4ccc4ccccc45)c4ccc(-c5cccc6ccccc56)c5oc6ccccc6c45)cc3)c(-c3ccccc3)c2)cc1] 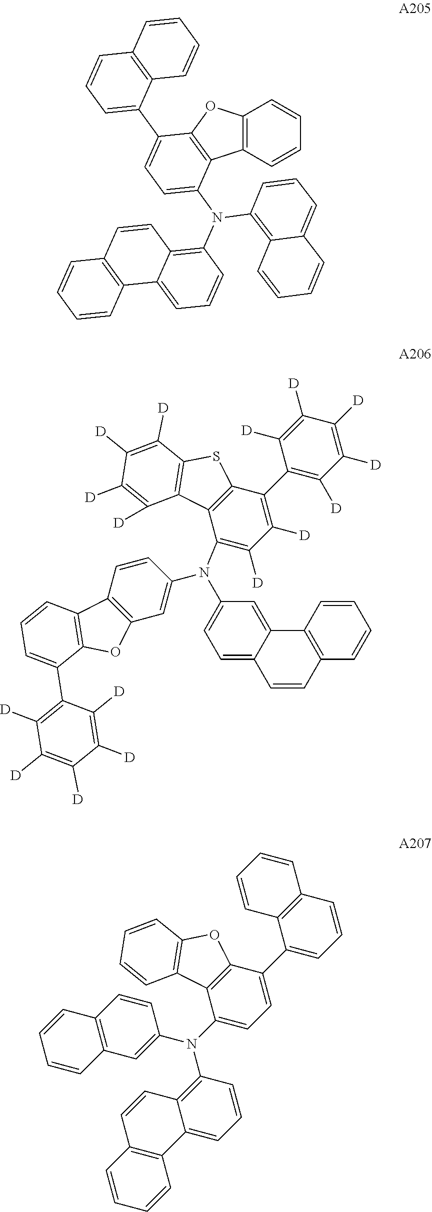 [SMILES: c1ccc2c(-c3ccc(N(c4cccc5ccccc45)c4cccc5c4ccc4ccccc45)c4c3oc3ccccc34)cccc2c1 ; [2H]c1c([2H])c([2H])c(-c2cccc3c2oc2cc(N(c4ccc5ccc6ccccc6c5c4)c4c([2H])c([2H])c(-c5c([2H])c([2H])c([2H])c([2H])c5[2H])c5sc6c([2H])c([2H])c([2H])c([2H])c6c45)ccc23)c([2H])c1[2H] ; c1ccc2cc(N(c3cccc4c3ccc3ccccc34)c3ccc(-c4cccc5ccccc45)c4oc5ccccc5c34)ccc2c1] 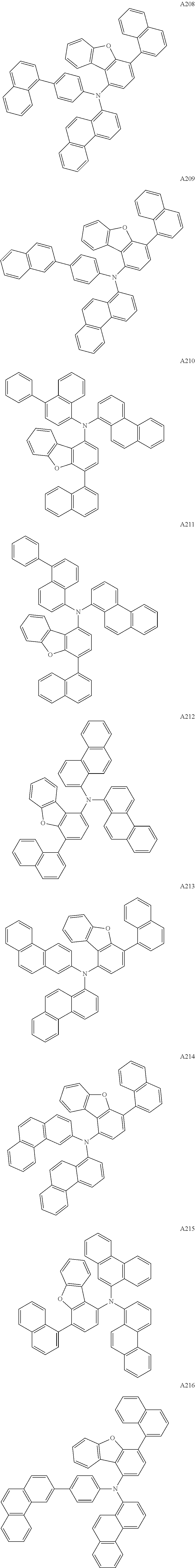 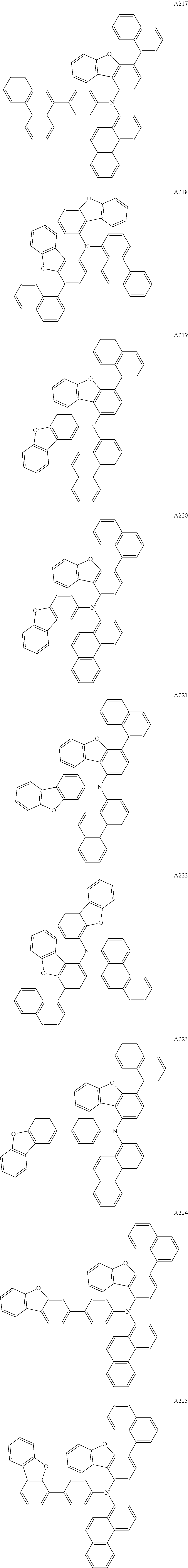 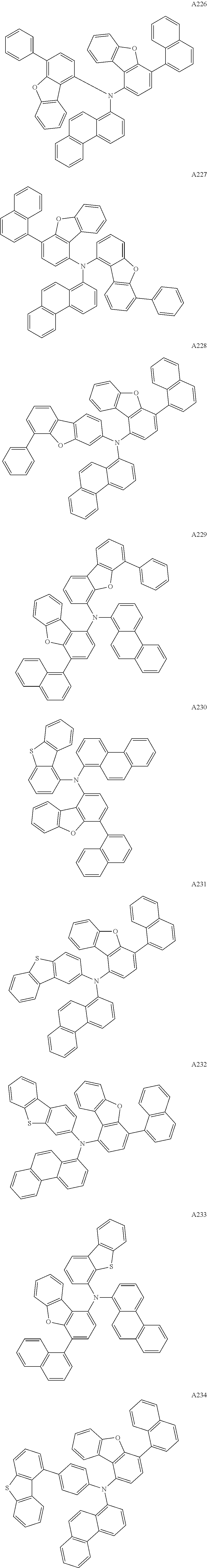 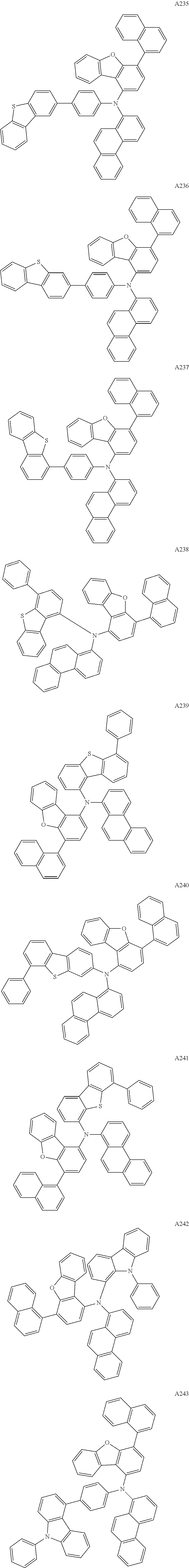 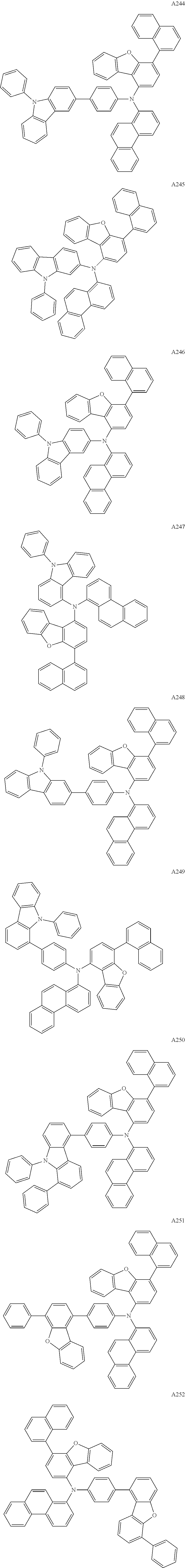 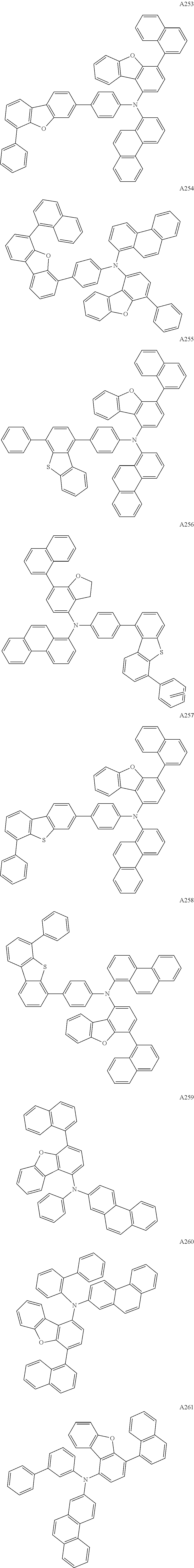 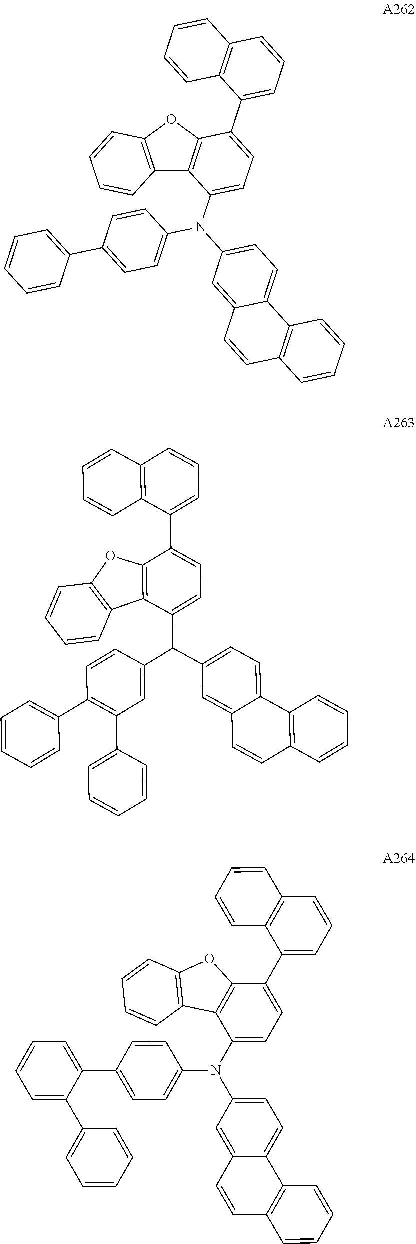 [SMILES: c1ccc(-c2ccc(N(c3ccc4c(ccc5ccccc54)c3)c3ccc(-c4cccc5ccccc45)c4oc5ccccc5c34)cc2)cc1 ; c1ccc(-c2ccc(C(c3ccc4c(ccc5ccccc54)c3)c3ccc(-c4cccc5ccccc45)c4oc5ccccc5c34)cc2-c2ccccc2)cc1 ; c1ccc(-c2ccccc2-c2ccc(N(c3ccc4c(ccc5ccccc54)c3)c3ccc(-c4cccc5ccccc45)c4oc5ccccc5c34)cc2)cc1] 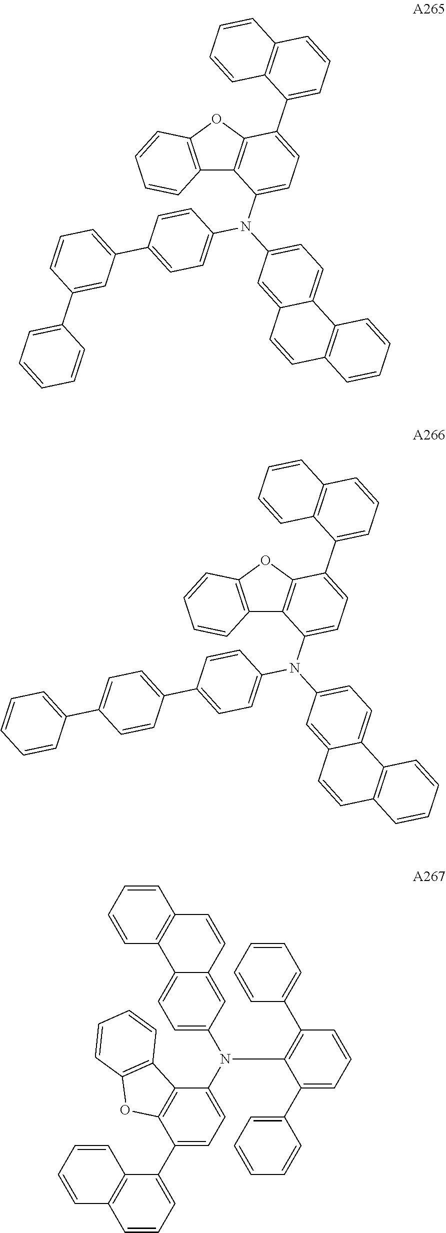 [SMILES: c1ccc(-c2cccc(-c3ccc(N(c4ccc5c(ccc6ccccc65)c4)c4ccc(-c5cccc6ccccc56)c5oc6ccccc6c45)cc3)c2)cc1 ; c1ccc(-c2ccc(-c3ccc(N(c4ccc5c(ccc6ccccc65)c4)c4ccc(-c5cccc6ccccc56)c5oc6ccccc6c45)cc3)cc2)cc1 ; c1ccc(-c2cccc(-c3ccccc3)c2N(c2ccc3c(ccc4ccccc43)c2)c2ccc(-c3cccc4ccccc34)c3oc4ccccc4c23)cc1] 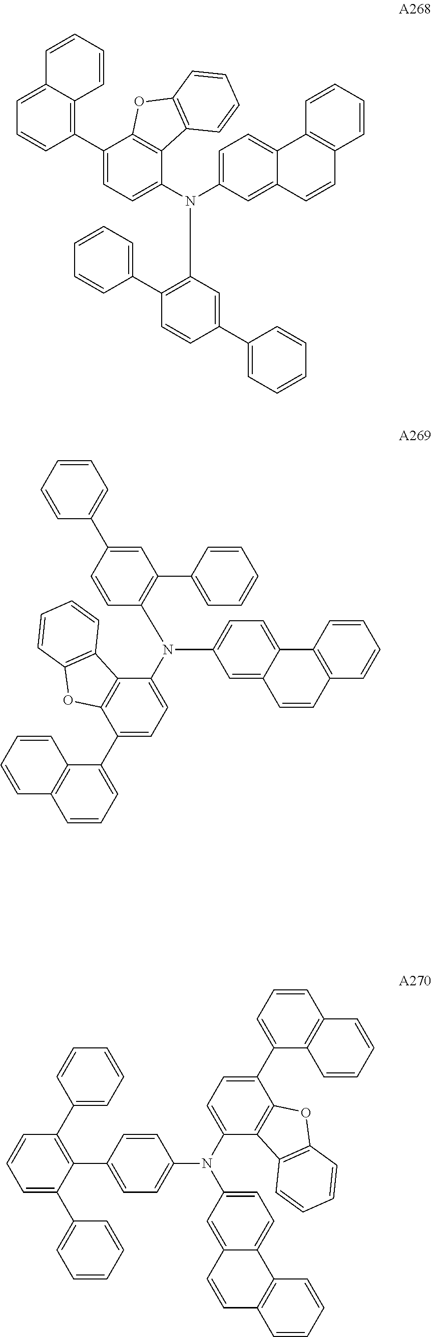 [SMILES: c1ccc(-c2ccc(-c3ccccc3)c(N(c3ccc4c(ccc5ccccc54)c3)c3ccc(-c4cccc5ccccc45)c4oc5ccccc5c34)c2)cc1 ; c1ccc(-c2ccc(N(c3ccc4c(ccc5ccccc54)c3)c3ccc(-c4cccc5ccccc45)c4oc5ccccc5c34)c(-c3ccccc3)c2)cc1 ; c1ccc(-c2cccc(-c3ccccc3)c2-c2ccc(N(c3ccc4c(ccc5ccccc54)c3)c3ccc(-c4cccc5ccccc45)c4oc5ccccc5c34)cc2)cc1] 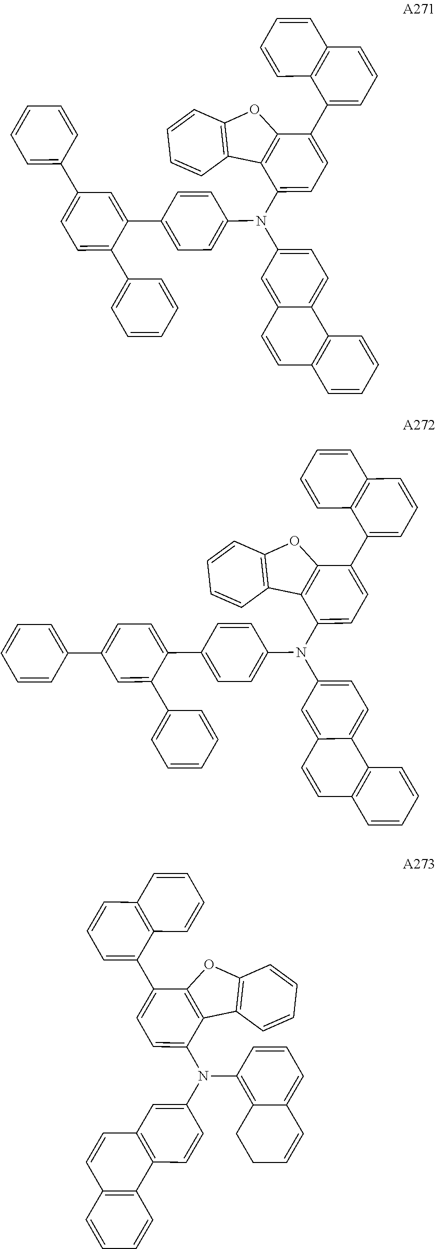 [SMILES: c1ccc(-c2ccc(-c3ccccc3)c(-c3ccc(N(c4ccc5c(ccc6ccccc65)c4)c4ccc(-c5cccc6ccccc56)c5oc6ccccc6c45)cc3)c2)cc1 ; c1ccc(-c2ccc(-c3ccc(N(c4ccc5c(ccc6ccccc65)c4)c4ccc(-c5cccc6ccccc56)c5oc6ccccc6c45)cc3)c(-c3ccccc3)c2)cc1 ; C1=Cc2cccc(N(c3ccc4c(ccc5ccccc54)c3)c3ccc(-c4cccc5ccccc45)c4oc5ccccc5c34)c2CC1] 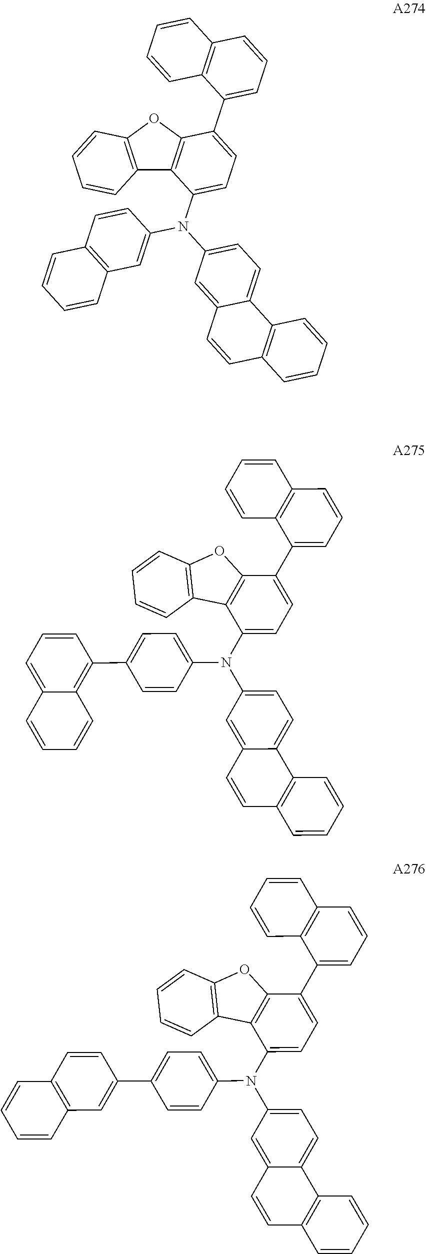 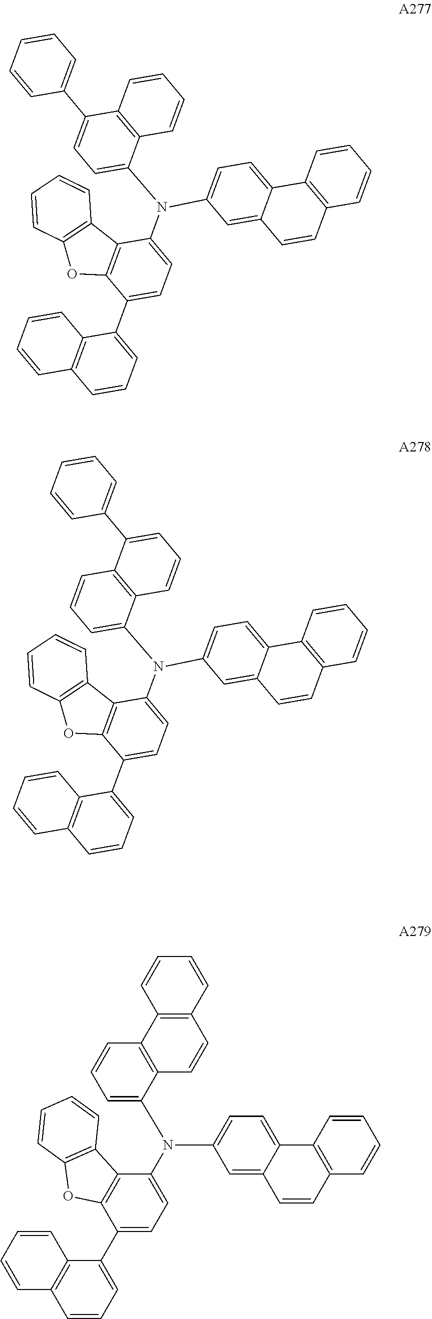 [SMILES: c1ccc(-c2ccc(N(c3ccc4c(ccc5ccccc54)c3)c3ccc(-c4cccc5ccccc45)c4oc5ccccc5c34)c3ccccc23)cc1 ; c1ccc(-c2cccc3c(N(c4ccc5c(ccc6ccccc65)c4)c4ccc(-c5cccc6ccccc56)c5oc6ccccc6c45)cccc23)cc1 ; c1ccc2c(-c3ccc(N(c4ccc5c(ccc6ccccc65)c4)c4cccc5c4ccc4ccccc45)c4c3oc3ccccc34)cccc2c1] 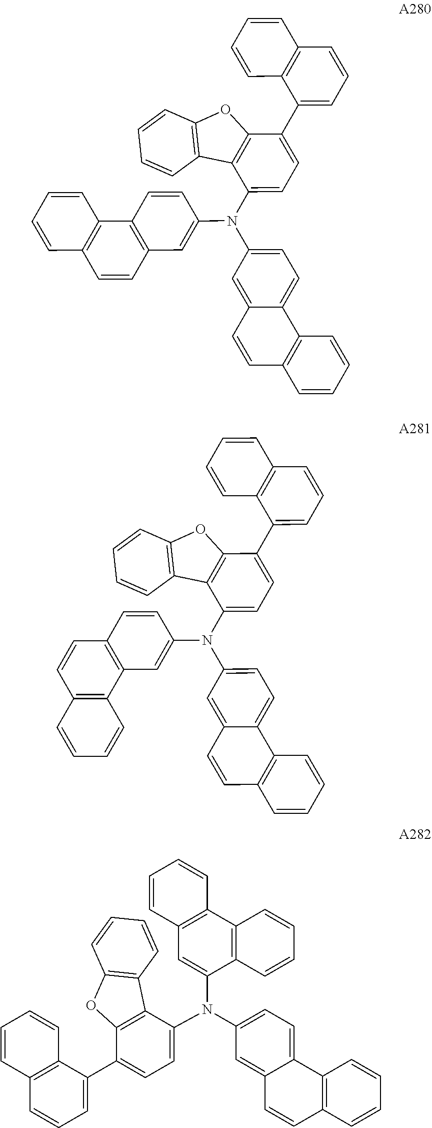 [SMILES: c1ccc2c(-c3ccc(N(c4ccc5c(ccc6ccccc65)c4)c4ccc5c(ccc6ccccc65)c4)c4c3oc3ccccc34)cccc2c1 ; c1ccc2c(-c3ccc(N(c4ccc5c(ccc6ccccc65)c4)c4ccc5ccc6ccccc6c5c4)c4c3oc3ccccc34)cccc2c1 ; c1ccc2c(-c3ccc(N(c4ccc5c(ccc6ccccc65)c4)c4cc5ccccc5c5ccccc45)c4c3oc3ccccc34)cccc2c1] 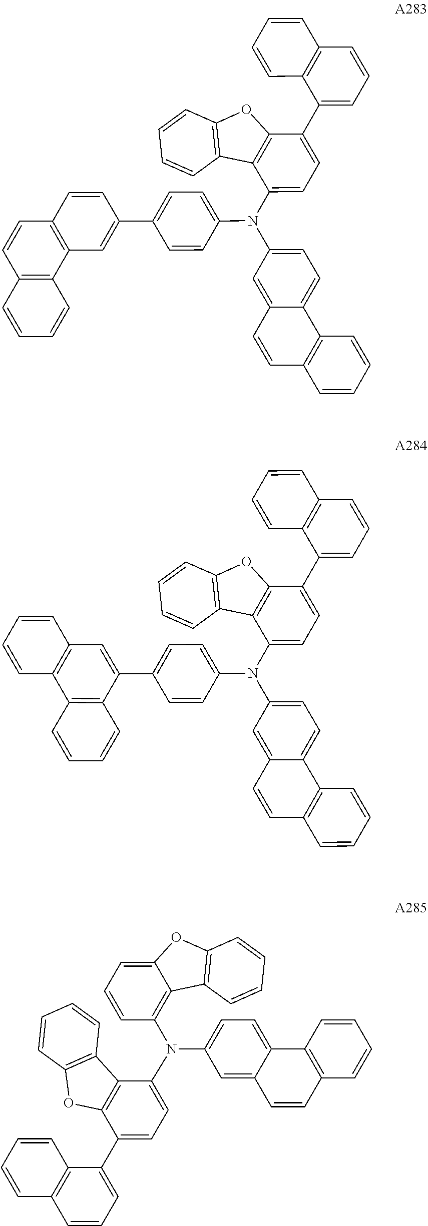 [SMILES: c1ccc2c(-c3ccc(N(c4ccc(-c5ccc6ccc7ccccc7c6c5)cc4)c4ccc5c(ccc6ccccc65)c4)c4c3oc3ccccc34)cccc2c1 ; c1ccc2c(-c3ccc(N(c4ccc(-c5cc6ccccc6c6ccccc56)cc4)c4ccc5c(ccc6ccccc65)c4)c4c3oc3ccccc34)cccc2c1 ; c1ccc2c(-c3ccc(N(c4ccc5c(ccc6ccccc65)c4)c4cccc5oc6ccccc6c45)c4c3oc3ccccc34)cccc2c1] 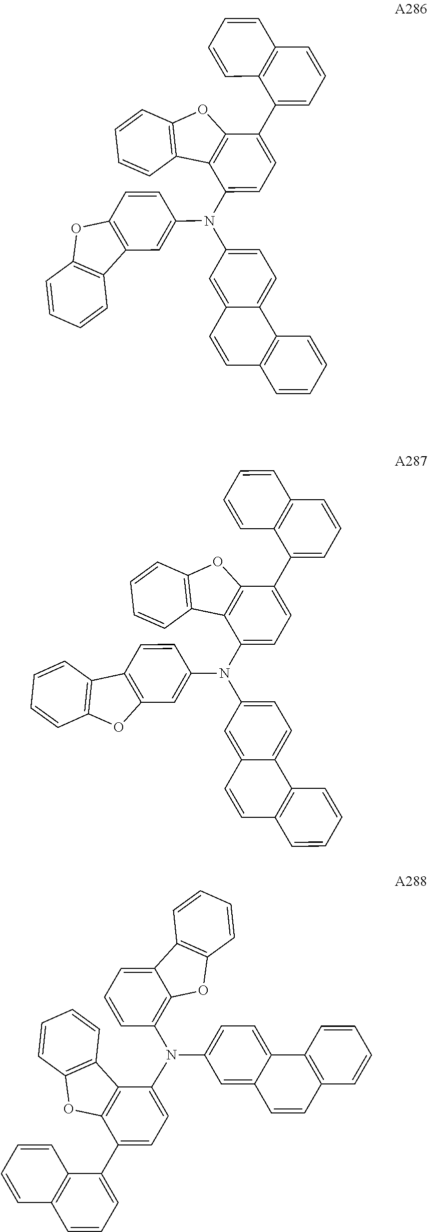 [SMILES: c1ccc2c(-c3ccc(N(c4ccc5c(ccc6ccccc65)c4)c4ccc5oc6ccccc6c5c4)c4c3oc3ccccc34)cccc2c1 ; c1ccc2c(-c3ccc(N(c4ccc5c(ccc6ccccc65)c4)c4ccc5c(c4)oc4ccccc45)c4c3oc3ccccc34)cccc2c1 ; c1ccc2c(-c3ccc(N(c4ccc5c(ccc6ccccc65)c4)c4cccc5c4oc4ccccc45)c4c3oc3ccccc34)cccc2c1] 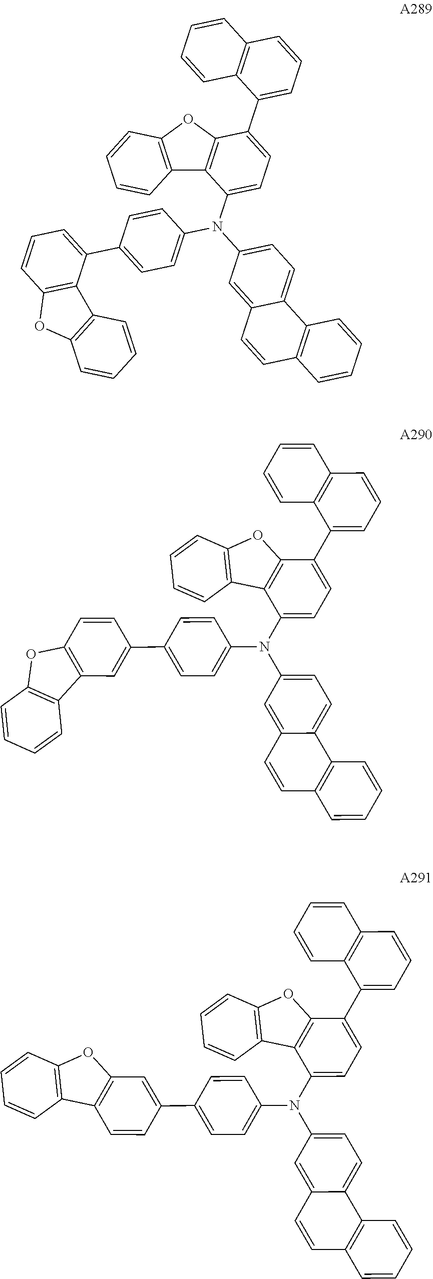 [SMILES: c1ccc2c(-c3ccc(N(c4ccc(-c5cccc6oc7ccccc7c56)cc4)c4ccc5c(ccc6ccccc65)c4)c4c3oc3ccccc34)cccc2c1 ; c1ccc2c(-c3ccc(N(c4ccc(-c5ccc6oc7ccccc7c6c5)cc4)c4ccc5c(ccc6ccccc65)c4)c4c3oc3ccccc34)cccc2c1 ; c1ccc2c(-c3ccc(N(c4ccc(-c5ccc6c(c5)oc5ccccc56)cc4)c4ccc5c(ccc6ccccc65)c4)c4c3oc3ccccc34)cccc2c1] 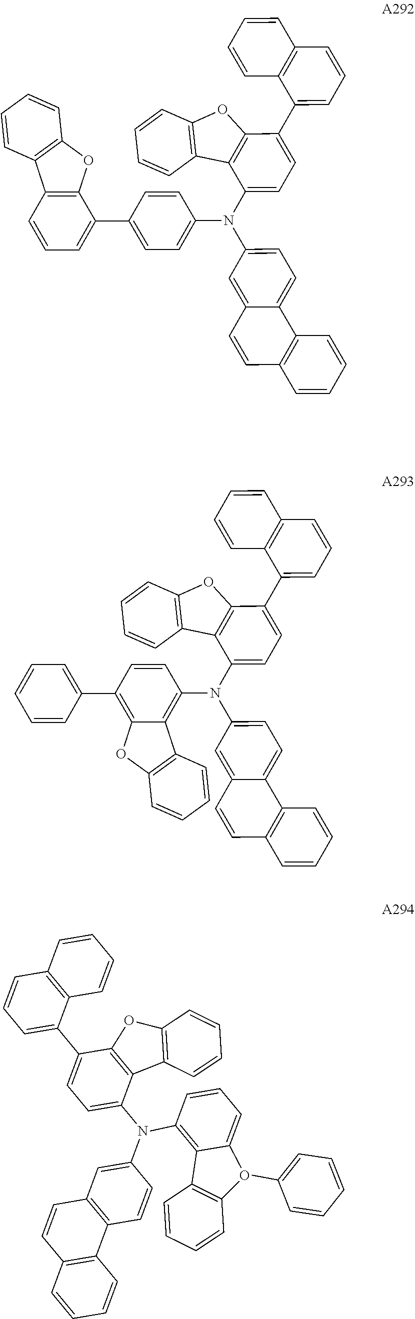 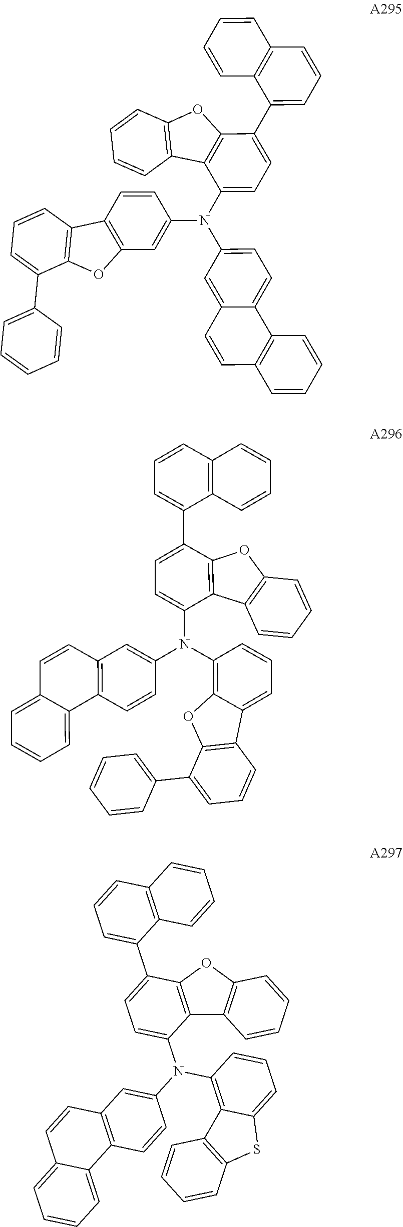 [SMILES: c1ccc(-c2cccc3c2oc2cc(N(c4ccc5c(ccc6ccccc65)c4)c4ccc(-c5cccc6ccccc56)c5oc6ccccc6c45)ccc23)cc1 ; c1ccc(-c2cccc3c2oc2c(N(c4ccc5c(ccc6ccccc65)c4)c4ccc(-c5cccc6ccccc56)c5oc6ccccc6c45)cccc23)cc1 ; c1ccc2c(-c3ccc(N(c4ccc5c(ccc6ccccc65)c4)c4cccc5sc6ccccc6c45)c4c3oc3ccccc34)cccc2c1] 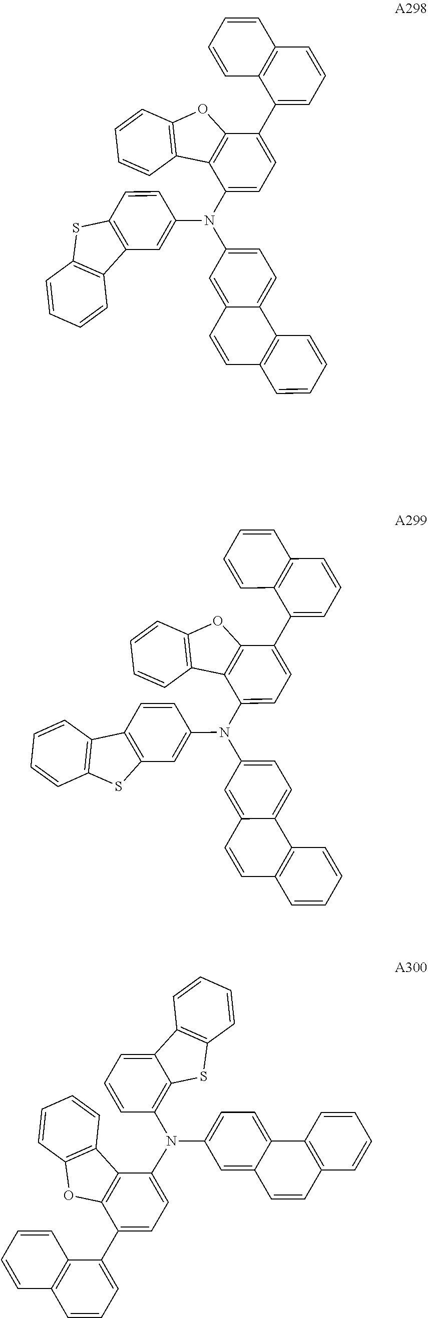 [SMILES: c1ccc2c(-c3ccc(N(c4ccc5c(ccc6ccccc65)c4)c4ccc5sc6ccccc6c5c4)c4c3oc3ccccc34)cccc2c1 ; c1ccc2c(-c3ccc(N(c4ccc5c(ccc6ccccc65)c4)c4ccc5c(c4)sc4ccccc45)c4c3oc3ccccc34)cccc2c1 ; c1ccc2c(-c3ccc(N(c4ccc5c(ccc6ccccc65)c4)c4cccc5c4sc4ccccc45)c4c3oc3ccccc34)cccc2c1] 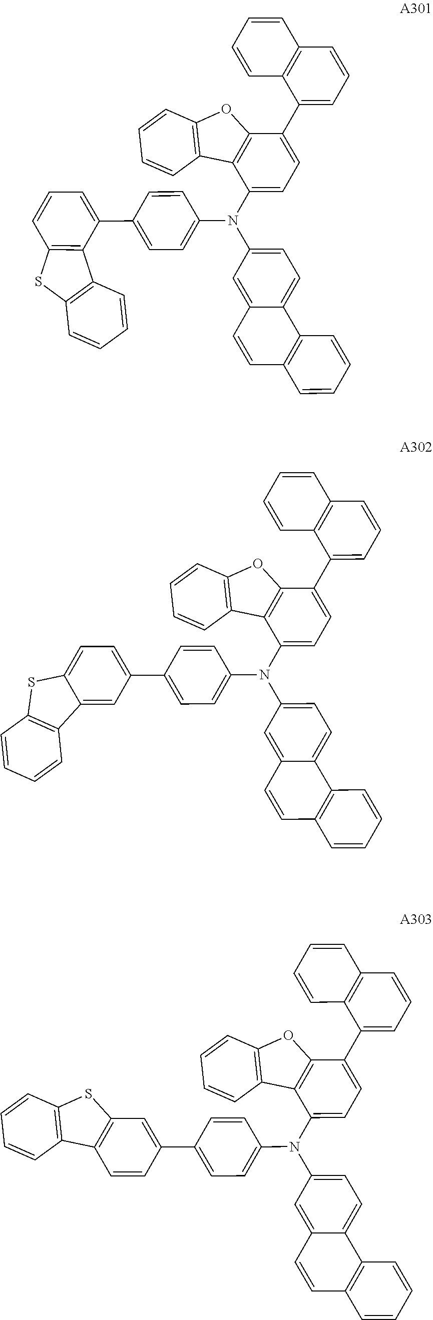 [SMILES: c1ccc2c(-c3ccc(N(c4ccc(-c5cccc6sc7ccccc7c56)cc4)c4ccc5c(ccc6ccccc65)c4)c4c3oc3ccccc34)cccc2c1 ; c1ccc2c(-c3ccc(N(c4ccc(-c5ccc6sc7ccccc7c6c5)cc4)c4ccc5c(ccc6ccccc65)c4)c4c3oc3ccccc34)cccc2c1 ; c1ccc2c(-c3ccc(N(c4ccc(-c5ccc6c(c5)sc5ccccc56)cc4)c4ccc5c(ccc6ccccc65)c4)c4c3oc3ccccc34)cccc2c1] 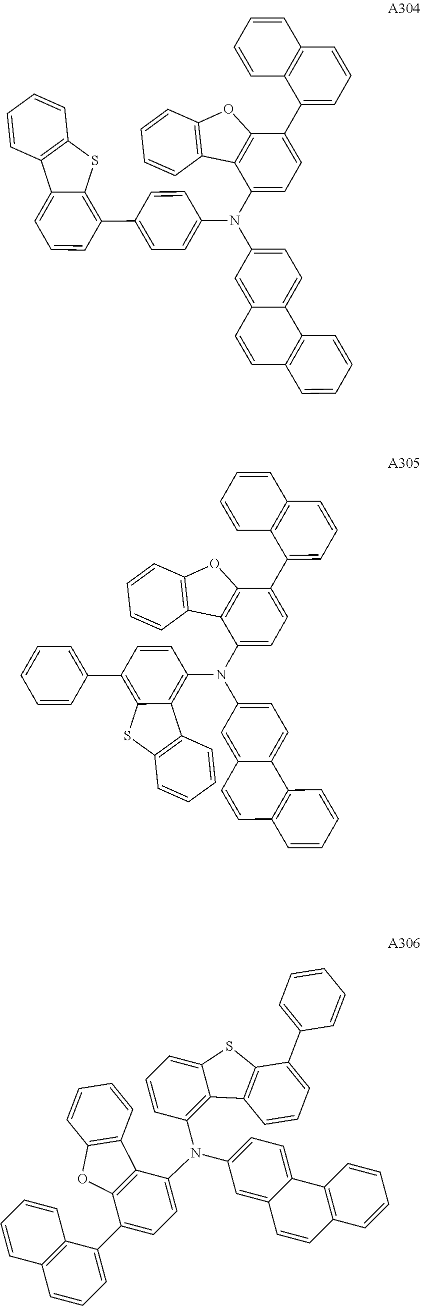 [SMILES: c1ccc2c(-c3ccc(N(c4ccc(-c5cccc6c5sc5ccccc56)cc4)c4ccc5c(ccc6ccccc65)c4)c4c3oc3ccccc34)cccc2c1 ; c1ccc(-c2ccc(N(c3ccc4c(ccc5ccccc54)c3)c3ccc(-c4cccc5ccccc45)c4oc5ccccc5c34)c3c2sc2ccccc23)cc1 ; c1ccc(-c2cccc3c2sc2cccc(N(c4ccc5c(ccc6ccccc65)c4)c4ccc(-c5cccc6ccccc56)c5oc6ccccc6c45)c23)cc1] 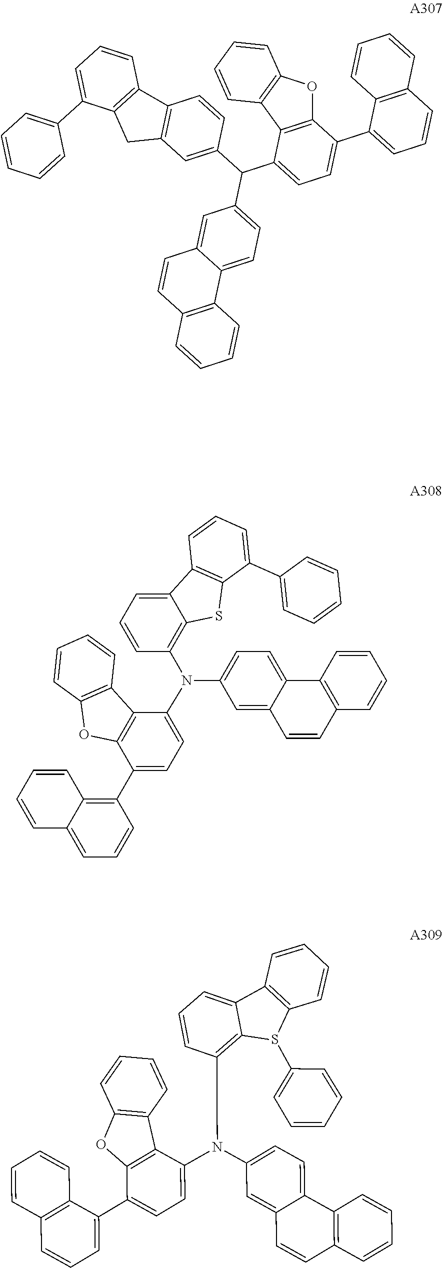 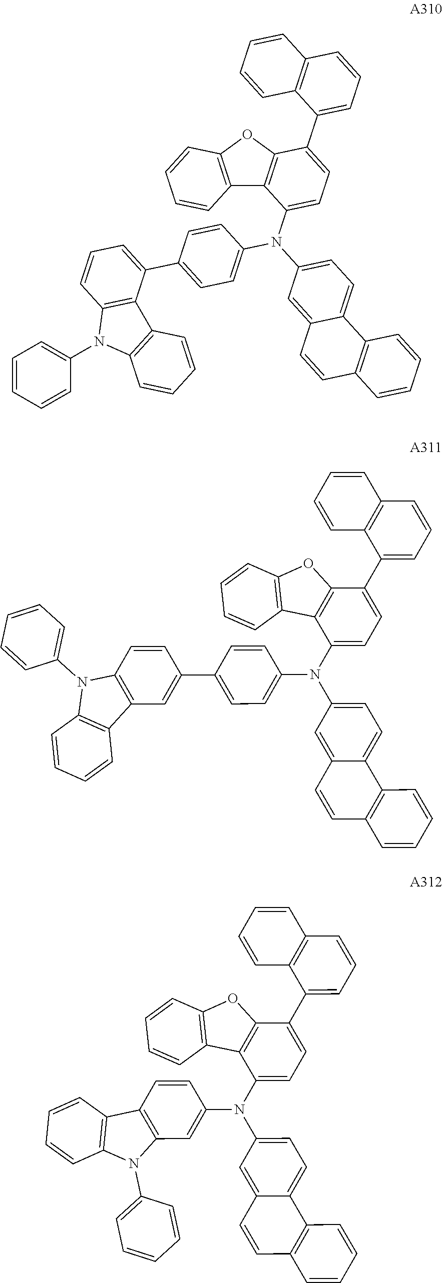 [SMILES: c1ccc(-n2c3ccccc3c3c(-c4ccc(N(c5ccc6c(ccc7ccccc76)c5)c5ccc(-c6cccc7ccccc67)c6oc7ccccc7c56)cc4)cccc32)cc1 ; c1ccc(-n2c3ccccc3c3cc(-c4ccc(N(c5ccc6c(ccc7ccccc76)c5)c5ccc(-c6cccc7ccccc67)c6oc7ccccc7c56)cc4)ccc32)cc1 ; c1ccc(-n2c3ccccc3c3ccc(N(c4ccc5c(ccc6ccccc65)c4)c4ccc(-c5cccc6ccccc56)c5oc6ccccc6c45)cc32)cc1] 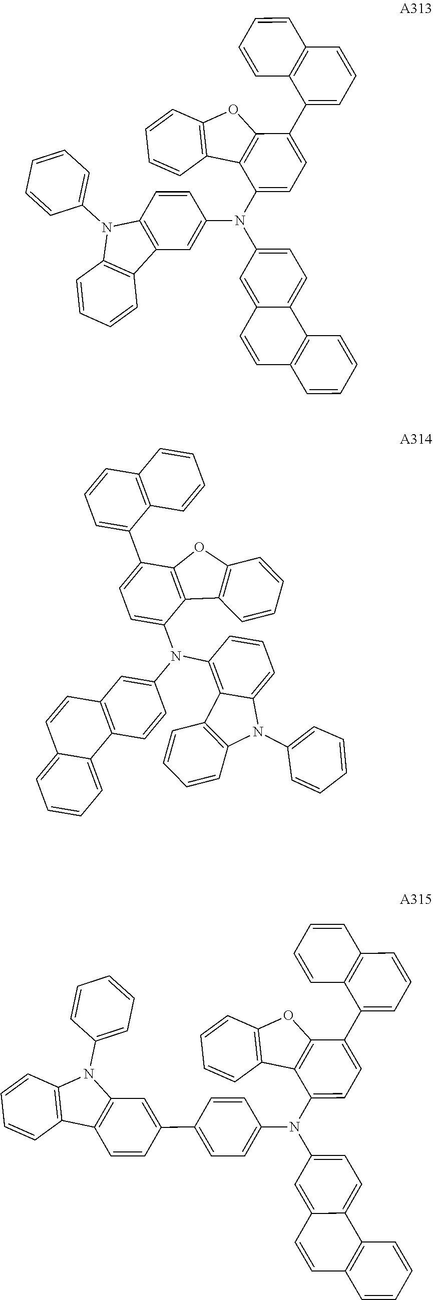 [SMILES: c1ccc(-n2c3ccccc3c3cc(N(c4ccc5c(ccc6ccccc65)c4)c4ccc(-c5cccc6ccccc56)c5oc6ccccc6c45)ccc32)cc1 ; c1ccc(-n2c3ccccc3c3c(N(c4ccc5c(ccc6ccccc65)c4)c4ccc(-c5cccc6ccccc56)c5oc6ccccc6c45)cccc32)cc1 ; c1ccc(-n2c3ccccc3c3ccc(-c4ccc(N(c5ccc6c(ccc7ccccc76)c5)c5ccc(-c6cccc7ccccc67)c6oc7ccccc7c56)cc4)cc32)cc1] 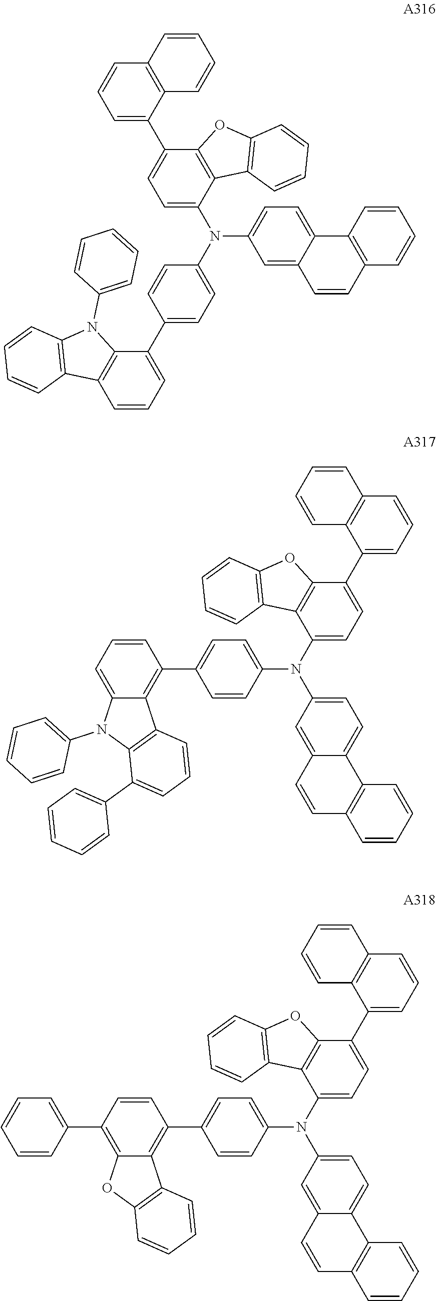 [SMILES: c1ccc(-n2c3ccccc3c3cccc(-c4ccc(N(c5ccc6c(ccc7ccccc76)c5)c5ccc(-c6cccc7ccccc67)c6oc7ccccc7c56)cc4)c32)cc1 ; c1ccc(-c2cccc3c4c(-c5ccc(N(c6ccc7c(ccc8ccccc87)c6)c6ccc(-c7cccc8ccccc78)c7oc8ccccc8c67)cc5)cccc4n(-c4ccccc4)c23)cc1 ; c1ccc(-c2ccc(-c3ccc(N(c4ccc5c(ccc6ccccc65)c4)c4ccc(-c5cccc6ccccc56)c5oc6ccccc6c45)cc3)c3c2oc2ccccc23)cc1] 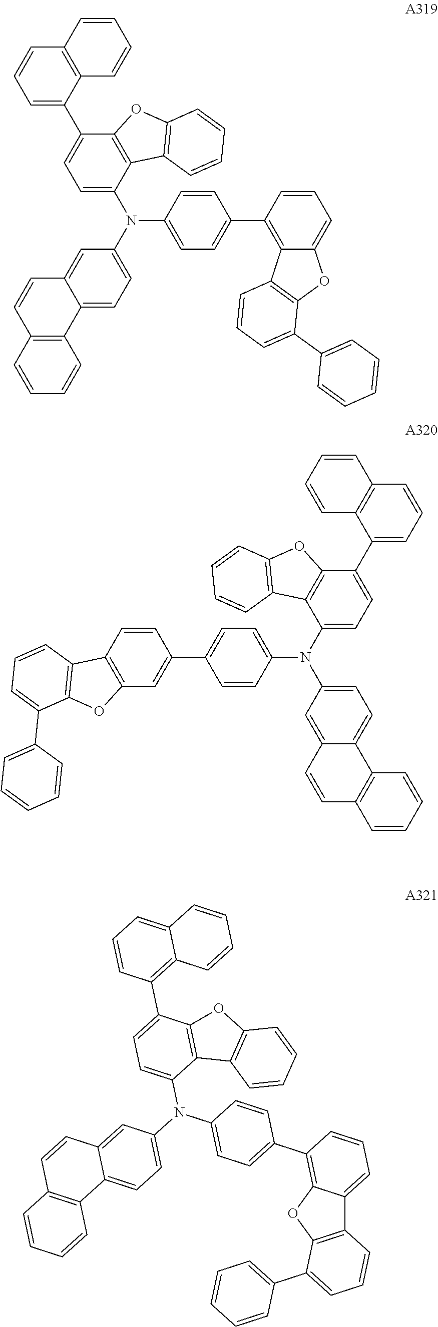 [SMILES: c1ccc(-c2cccc3c2oc2cccc(-c4ccc(N(c5ccc6c(ccc7ccccc76)c5)c5ccc(-c6cccc7ccccc67)c6oc7ccccc7c56)cc4)c23)cc1 ; c1ccc(-c2cccc3c2oc2cc(-c4ccc(N(c5ccc6c(ccc7ccccc76)c5)c5ccc(-c6cccc7ccccc67)c6oc7ccccc7c56)cc4)ccc23)cc1 ; c1ccc(-c2cccc3c2oc2c(-c4ccc(N(c5ccc6c(ccc7ccccc76)c5)c5ccc(-c6cccc7ccccc67)c6oc7ccccc7c56)cc4)cccc23)cc1] 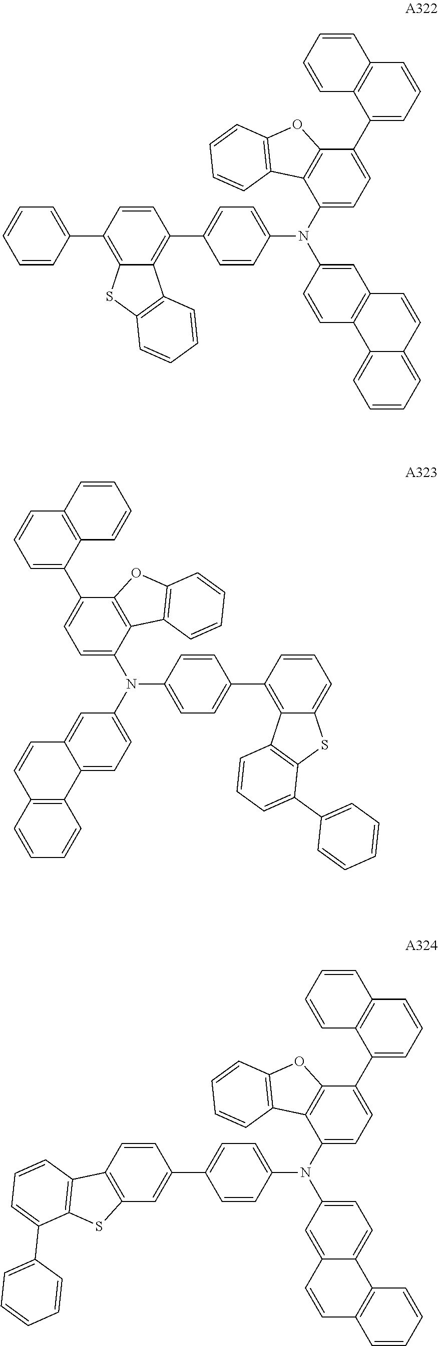 [SMILES: c1ccc(-c2ccc(-c3ccc(N(c4ccc5c(ccc6ccccc65)c4)c4ccc(-c5cccc6ccccc56)c5oc6ccccc6c45)cc3)c3c2sc2ccccc23)cc1 ; c1ccc(-c2cccc3c2sc2cccc(-c4ccc(N(c5ccc6c(ccc7ccccc76)c5)c5ccc(-c6cccc7ccccc67)c6oc7ccccc7c56)cc4)c23)cc1 ; c1ccc(-c2cccc3c2sc2cc(-c4ccc(N(c5ccc6c(ccc7ccccc76)c5)c5ccc(-c6cccc7ccccc67)c6oc7ccccc7c56)cc4)ccc23)cc1] 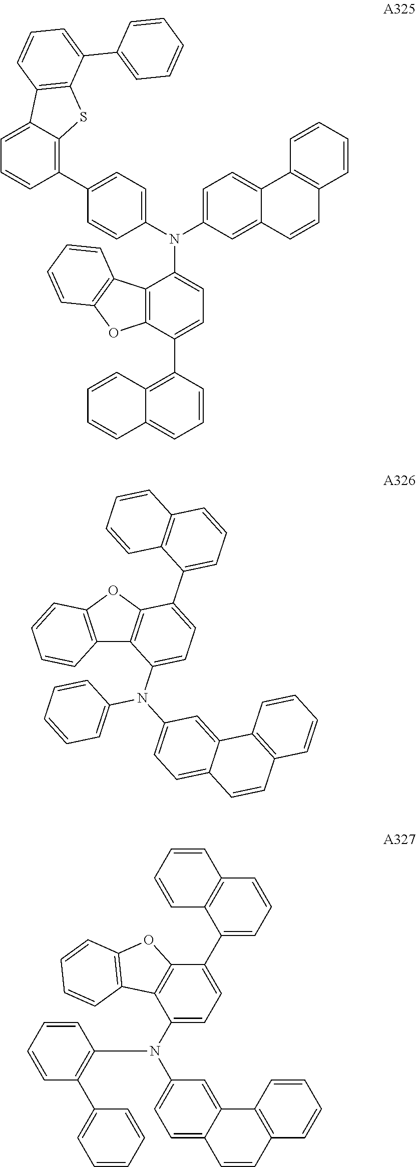 [SMILES: c1ccc(-c2cccc3c2sc2c(-c4ccc(N(c5ccc6c(ccc7ccccc76)c5)c5ccc(-c6cccc7ccccc67)c6oc7ccccc7c56)cc4)cccc23)cc1 ; c1ccc(N(c2ccc3ccc4ccccc4c3c2)c2ccc(-c3cccc4ccccc34)c3oc4ccccc4c23)cc1 ; c1ccc(-c2ccccc2N(c2ccc3ccc4ccccc4c3c2)c2ccc(-c3cccc4ccccc34)c3oc4ccccc4c23)cc1] 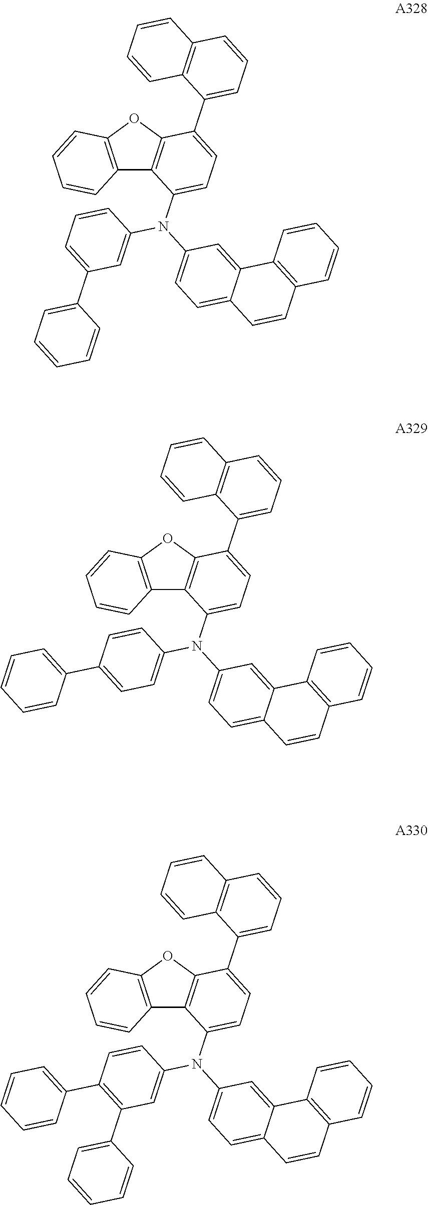 [SMILES: c1ccc(-c2cccc(N(c3ccc4ccc5ccccc5c4c3)c3ccc(-c4cccc5ccccc45)c4oc5ccccc5c34)c2)cc1 ; c1ccc(-c2ccc(N(c3ccc4ccc5ccccc5c4c3)c3ccc(-c4cccc5ccccc45)c4oc5ccccc5c34)cc2)cc1 ; c1ccc(-c2ccc(N(c3ccc4ccc5ccccc5c4c3)c3ccc(-c4cccc5ccccc45)c4oc5ccccc5c34)cc2-c2ccccc2)cc1] 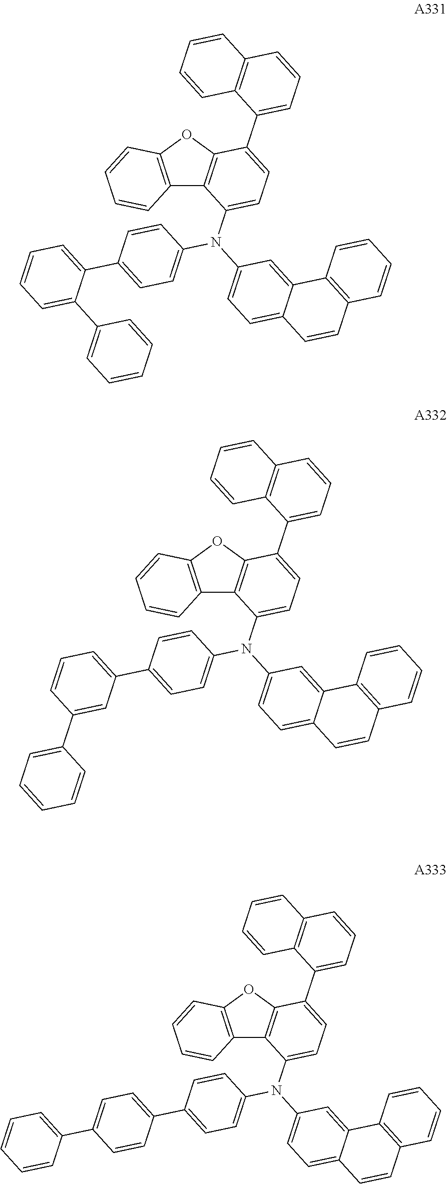 [SMILES: c1ccc(-c2ccccc2-c2ccc(N(c3ccc4ccc5ccccc5c4c3)c3ccc(-c4cccc5ccccc45)c4oc5ccccc5c34)cc2)cc1 ; c1ccc(-c2cccc(-c3ccc(N(c4ccc5ccc6ccccc6c5c4)c4ccc(-c5cccc6ccccc56)c5oc6ccccc6c45)cc3)c2)cc1 ; c1ccc(-c2ccc(-c3ccc(N(c4ccc5ccc6ccccc6c5c4)c4ccc(-c5cccc6ccccc56)c5oc6ccccc6c45)cc3)cc2)cc1] 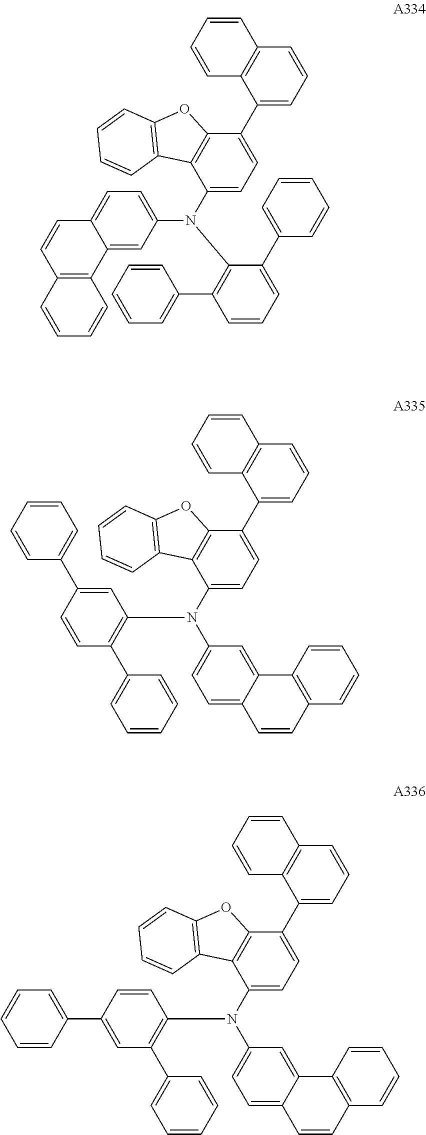 [SMILES: c1ccc(-c2cccc(-c3ccccc3)c2N(c2ccc3ccc4ccccc4c3c2)c2ccc(-c3cccc4ccccc34)c3oc4ccccc4c23)cc1 ; c1ccc(-c2ccc(-c3ccccc3)c(N(c3ccc4ccc5ccccc5c4c3)c3ccc(-c4cccc5ccccc45)c4oc5ccccc5c34)c2)cc1 ; c1ccc(-c2ccc(N(c3ccc4ccc5ccccc5c4c3)c3ccc(-c4cccc5ccccc45)c4oc5ccccc5c34)c(-c3ccccc3)c2)cc1] 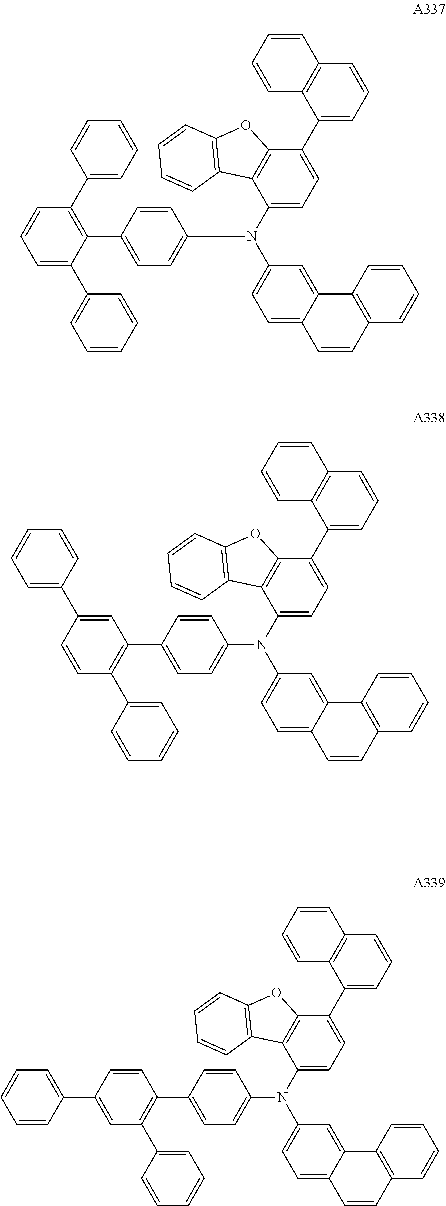 [SMILES: c1ccc(-c2cccc(-c3ccccc3)c2-c2ccc(N(c3ccc4ccc5ccccc5c4c3)c3ccc(-c4cccc5ccccc45)c4oc5ccccc5c34)cc2)cc1 ; c1ccc(-c2ccc(-c3ccccc3)c(-c3ccc(N(c4ccc5ccc6ccccc6c5c4)c4ccc(-c5cccc6ccccc56)c5oc6ccccc6c45)cc3)c2)cc1 ; c1ccc(-c2ccc(-c3ccc(N(c4ccc5ccc6ccccc6c5c4)c4ccc(-c5cccc6ccccc56)c5oc6ccccc6c45)cc3)c(-c3ccccc3)c2)cc1] 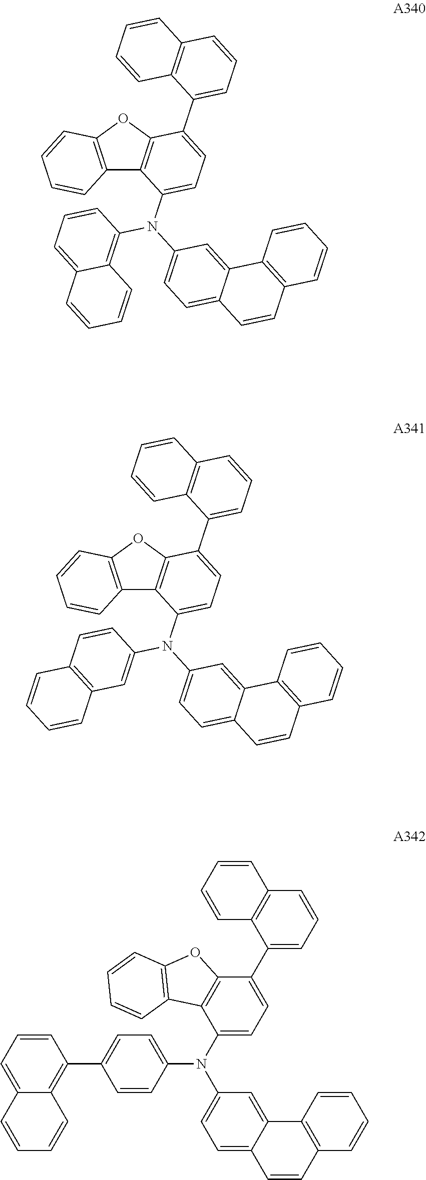 [SMILES: c1ccc2c(-c3ccc(N(c4ccc5ccc6ccccc6c5c4)c4cccc5ccccc45)c4c3oc3ccccc34)cccc2c1 ; c1ccc2cc(N(c3ccc4ccc5ccccc5c4c3)c3ccc(-c4cccc5ccccc45)c4oc5ccccc5c34)ccc2c1 ; c1ccc2c(-c3ccc(N(c4ccc5ccc6ccccc6c5c4)c4ccc(-c5cccc6ccccc56)c5oc6ccccc6c45)cc3)cccc2c1] 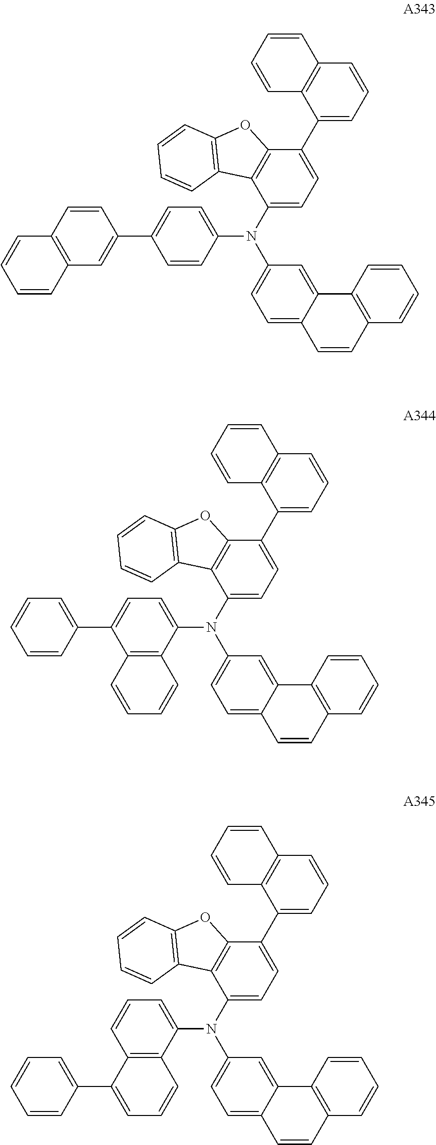 [SMILES: c1ccc2cc(-c3ccc(N(c4ccc5ccc6ccccc6c5c4)c4ccc(-c5cccc6ccccc56)c5oc6ccccc6c45)cc3)ccc2c1 ; c1ccc(-c2ccc(N(c3ccc4ccc5ccccc5c4c3)c3ccc(-c4cccc5ccccc45)c4oc5ccccc5c34)c3ccccc23)cc1 ; c1ccc(-c2cccc3c(N(c4ccc5ccc6ccccc6c5c4)c4ccc(-c5cccc6ccccc56)c5oc6ccccc6c45)cccc23)cc1] 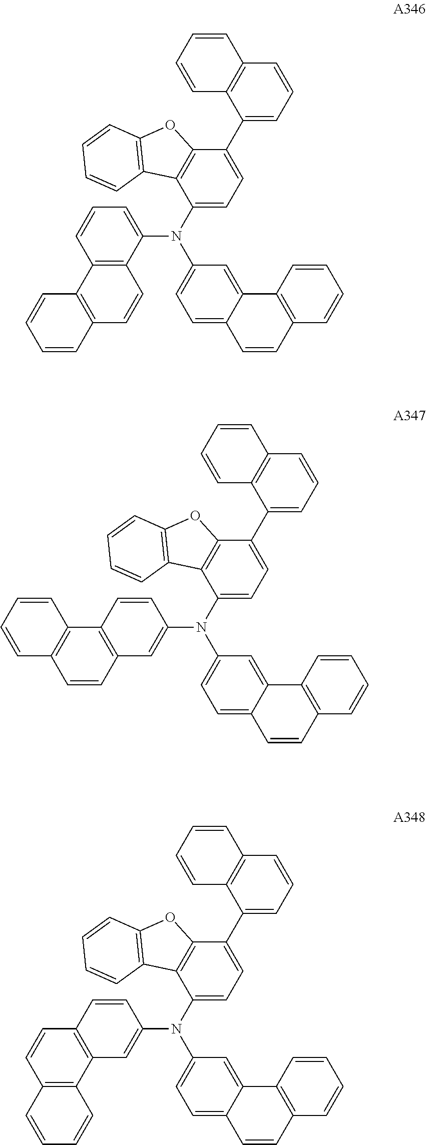 [SMILES: c1ccc2c(-c3ccc(N(c4ccc5ccc6ccccc6c5c4)c4cccc5c4ccc4ccccc45)c4c3oc3ccccc34)cccc2c1 ; c1ccc2c(-c3ccc(N(c4ccc5c(ccc6ccccc65)c4)c4ccc5ccc6ccccc6c5c4)c4c3oc3ccccc34)cccc2c1 ; c1ccc2c(-c3ccc(N(c4ccc5ccc6ccccc6c5c4)c4ccc5ccc6ccccc6c5c4)c4c3oc3ccccc34)cccc2c1] 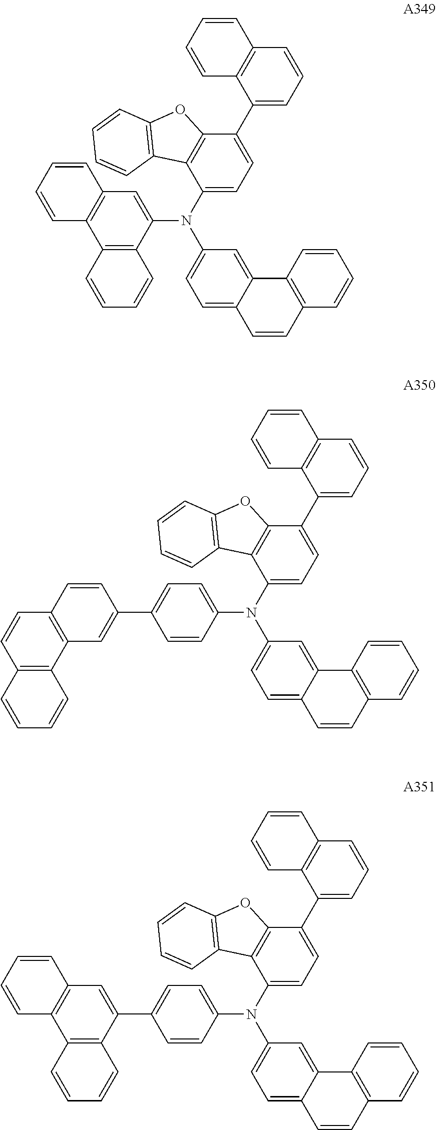 [SMILES: c1ccc2c(-c3ccc(N(c4ccc5ccc6ccccc6c5c4)c4cc5ccccc5c5ccccc45)c4c3oc3ccccc34)cccc2c1 ; c1ccc2c(-c3ccc(N(c4ccc(-c5ccc6ccc7ccccc7c6c5)cc4)c4ccc5ccc6ccccc6c5c4)c4c3oc3ccccc34)cccc2c1 ; c1ccc2c(-c3ccc(N(c4ccc(-c5cc6ccccc6c6ccccc56)cc4)c4ccc5ccc6ccccc6c5c4)c4c3oc3ccccc34)cccc2c1] 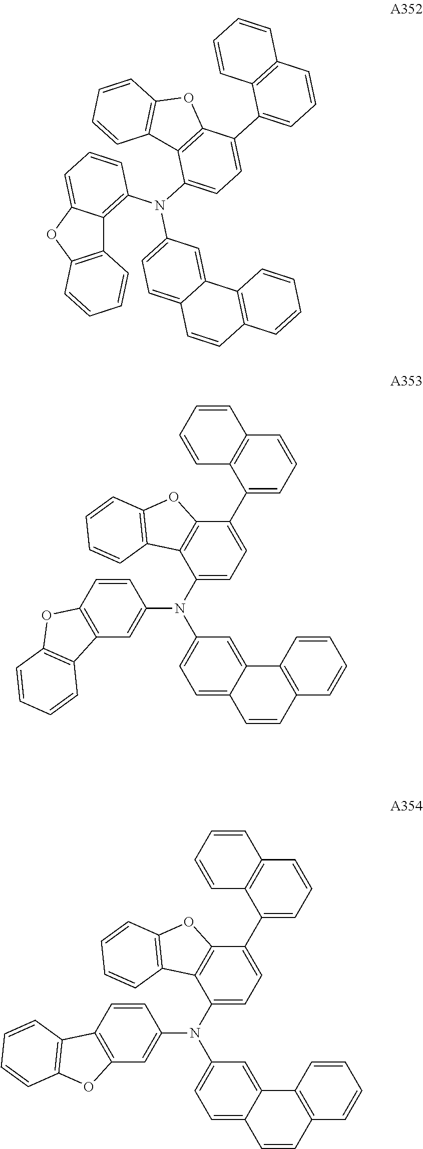 [SMILES: c1ccc2c(-c3ccc(N(c4ccc5ccc6ccccc6c5c4)c4cccc5oc6ccccc6c45)c4c3oc3ccccc34)cccc2c1 ; c1ccc2c(-c3ccc(N(c4ccc5ccc6ccccc6c5c4)c4ccc5oc6ccccc6c5c4)c4c3oc3ccccc34)cccc2c1 ; c1ccc2c(-c3ccc(N(c4ccc5c(c4)oc4ccccc45)c4ccc5ccc6ccccc6c5c4)c4c3oc3ccccc34)cccc2c1] 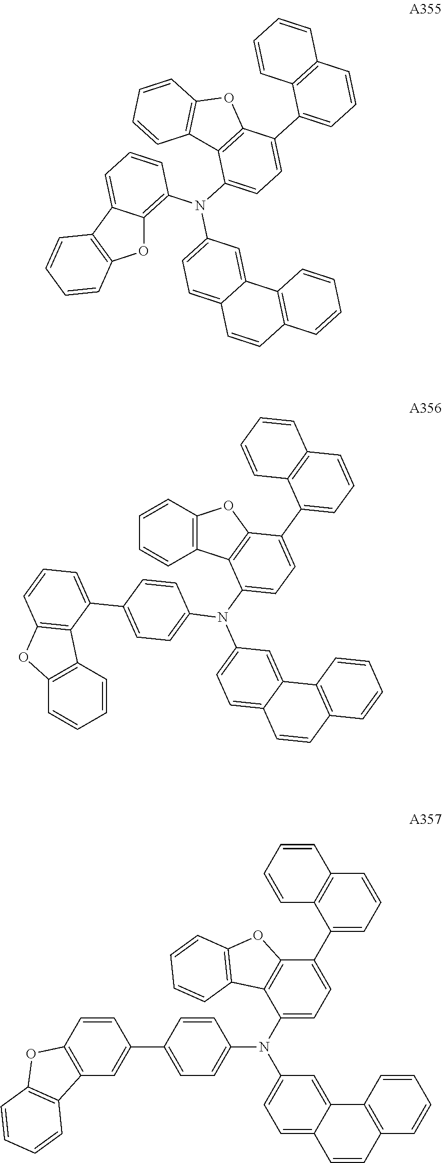 [SMILES: c1ccc2c(-c3ccc(N(c4ccc5ccc6ccccc6c5c4)c4cccc5c4oc4ccccc45)c4c3oc3ccccc34)cccc2c1 ; c1ccc2c(-c3ccc(N(c4ccc(-c5cccc6oc7ccccc7c56)cc4)c4ccc5ccc6ccccc6c5c4)c4c3oc3ccccc34)cccc2c1 ; c1ccc2c(-c3ccc(N(c4ccc(-c5ccc6oc7ccccc7c6c5)cc4)c4ccc5ccc6ccccc6c5c4)c4c3oc3ccccc34)cccc2c1] 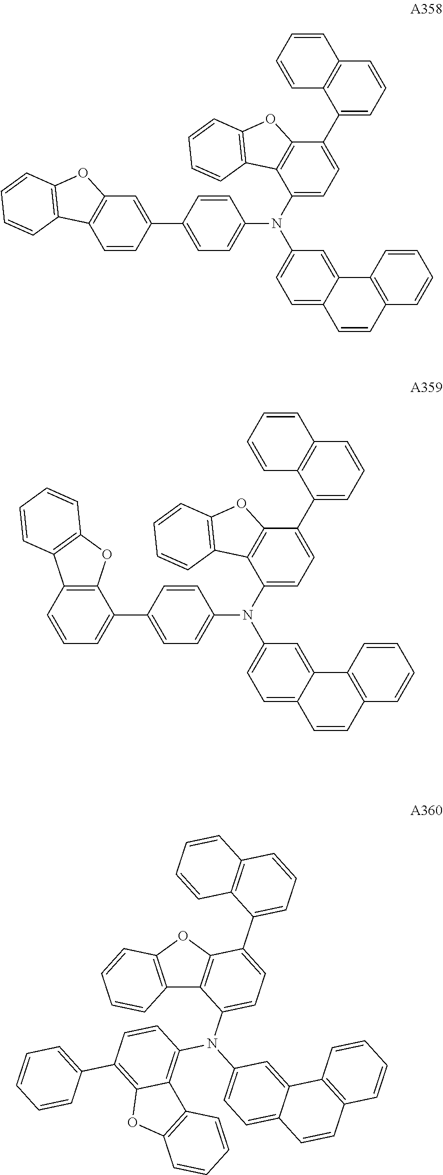 [SMILES: c1ccc2c(-c3ccc(N(c4ccc(-c5ccc6c(c5)oc5ccccc56)cc4)c4ccc5ccc6ccccc6c5c4)c4c3oc3ccccc34)cccc2c1 ; c1ccc2c(-c3ccc(N(c4ccc(-c5cccc6c5oc5ccccc56)cc4)c4ccc5ccc6ccccc6c5c4)c4c3oc3ccccc34)cccc2c1 ; c1ccc(-c2ccc(N(c3ccc4ccc5ccccc5c4c3)c3ccc(-c4cccc5ccccc45)c4oc5ccccc5c34)c3c2oc2ccccc23)cc1] 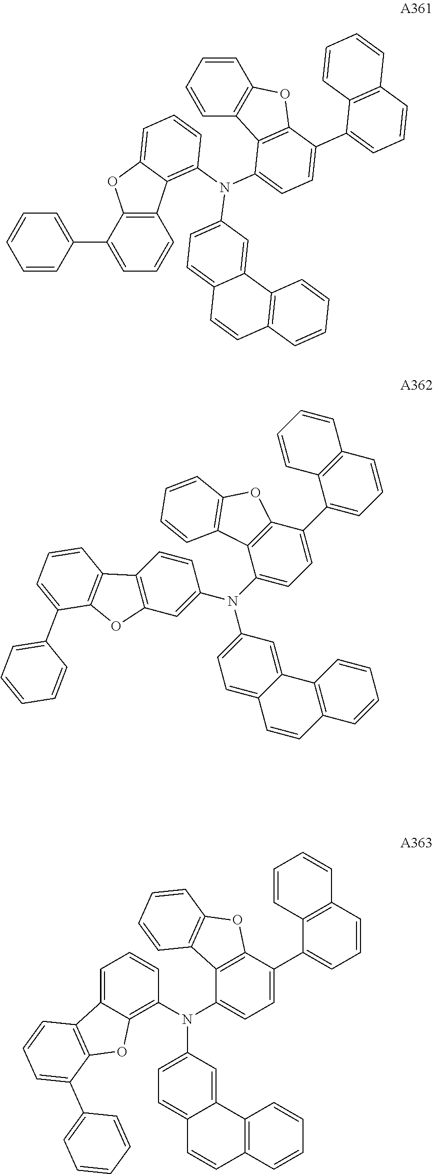 [SMILES: c1ccc(-c2cccc3c2oc2cccc(N(c4ccc5ccc6ccccc6c5c4)c4ccc(-c5cccc6ccccc56)c5oc6ccccc6c45)c23)cc1 ; c1ccc(-c2cccc3c2oc2cc(N(c4ccc5ccc6ccccc6c5c4)c4ccc(-c5cccc6ccccc56)c5oc6ccccc6c45)ccc23)cc1 ; c1ccc(-c2cccc3c2oc2c(N(c4ccc5ccc6ccccc6c5c4)c4ccc(-c5cccc6ccccc56)c5oc6ccccc6c45)cccc23)cc1] 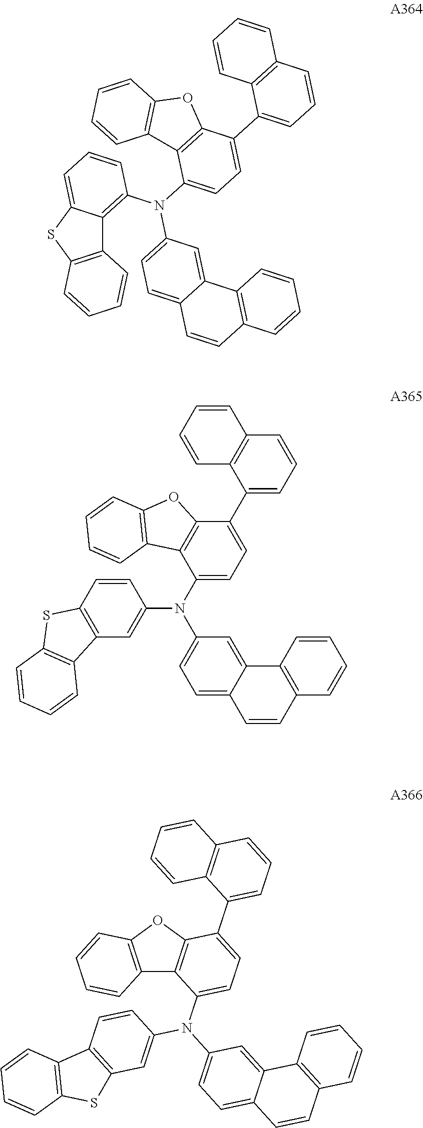 [SMILES: c1ccc2c(-c3ccc(N(c4ccc5ccc6ccccc6c5c4)c4cccc5sc6ccccc6c45)c4c3oc3ccccc34)cccc2c1 ; c1ccc2c(-c3ccc(N(c4ccc5ccc6ccccc6c5c4)c4ccc5sc6ccccc6c5c4)c4c3oc3ccccc34)cccc2c1 ; c1ccc2c(-c3ccc(N(c4ccc5c(c4)sc4ccccc45)c4ccc5ccc6ccccc6c5c4)c4c3oc3ccccc34)cccc2c1] 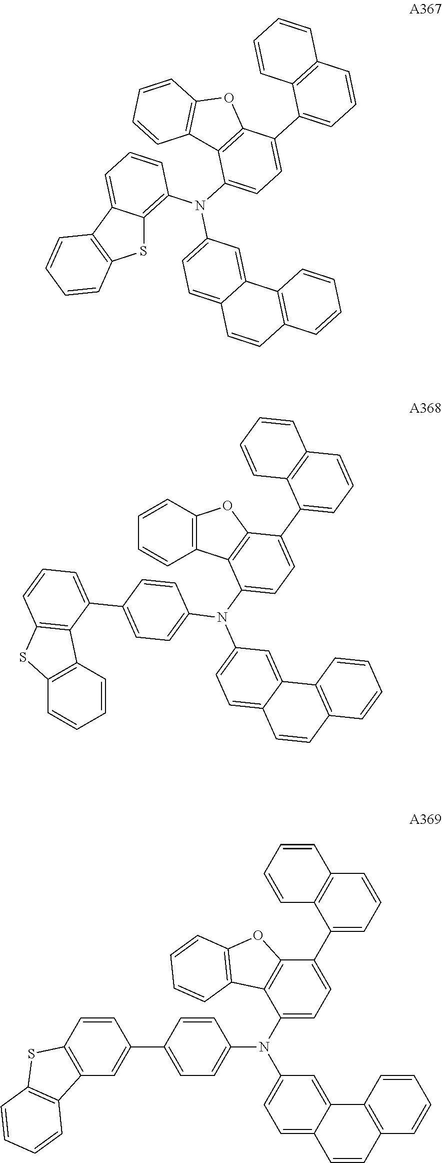 [SMILES: c1ccc2c(-c3ccc(N(c4ccc5ccc6ccccc6c5c4)c4cccc5c4sc4ccccc45)c4c3oc3ccccc34)cccc2c1 ; c1ccc2c(-c3ccc(N(c4ccc(-c5cccc6sc7ccccc7c56)cc4)c4ccc5ccc6ccccc6c5c4)c4c3oc3ccccc34)cccc2c1 ; c1ccc2c(-c3ccc(N(c4ccc(-c5ccc6sc7ccccc7c6c5)cc4)c4ccc5ccc6ccccc6c5c4)c4c3oc3ccccc34)cccc2c1] 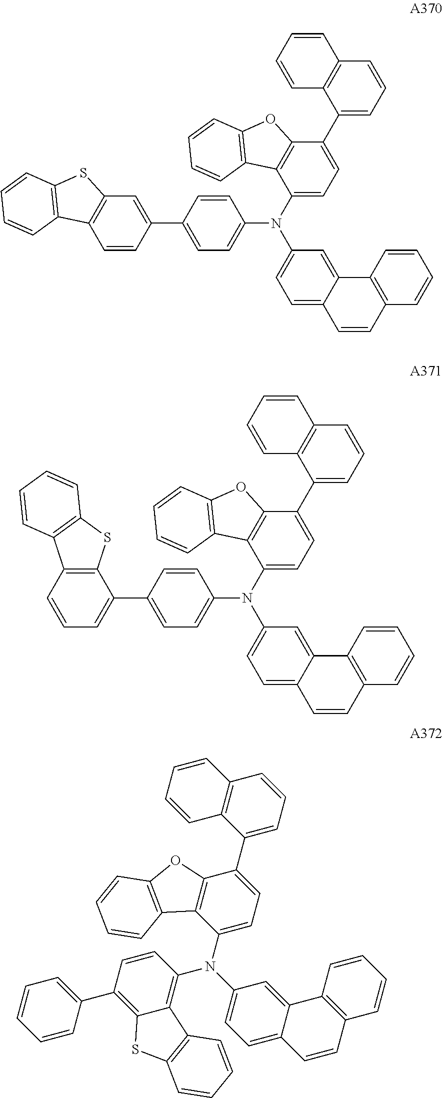 [SMILES: c1ccc2c(-c3ccc(N(c4ccc(-c5ccc6c(c5)sc5ccccc56)cc4)c4ccc5ccc6ccccc6c5c4)c4c3oc3ccccc34)cccc2c1 ; c1ccc2c(-c3ccc(N(c4ccc(-c5cccc6c5sc5ccccc56)cc4)c4ccc5ccc6ccccc6c5c4)c4c3oc3ccccc34)cccc2c1 ; c1ccc(-c2ccc(N(c3ccc4ccc5ccccc5c4c3)c3ccc(-c4cccc5ccccc45)c4oc5ccccc5c34)c3c2sc2ccccc23)cc1] 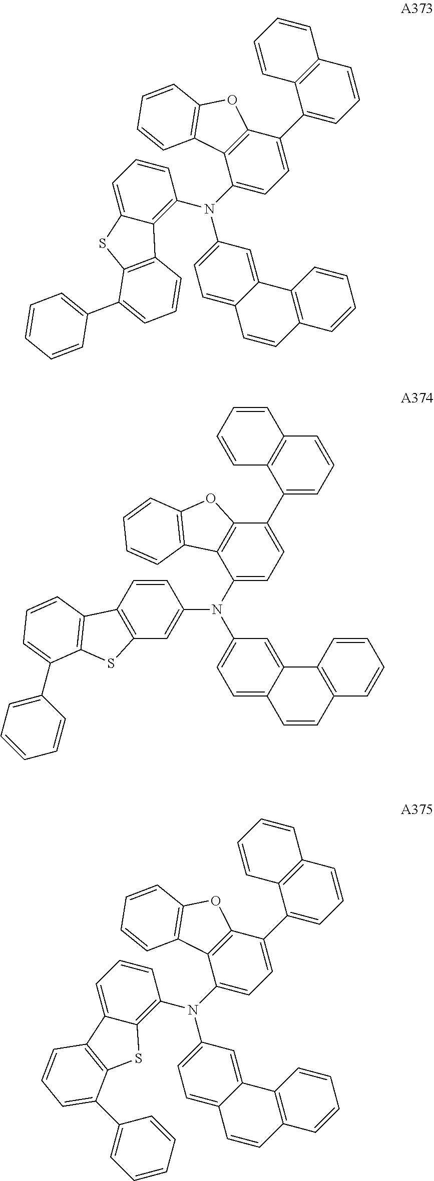 [SMILES: c1ccc(-c2cccc3c2sc2cccc(N(c4ccc5ccc6ccccc6c5c4)c4ccc(-c5cccc6ccccc56)c5oc6ccccc6c45)c23)cc1 ; c1ccc(-c2cccc3c2sc2cc(N(c4ccc5ccc6ccccc6c5c4)c4ccc(-c5cccc6ccccc56)c5oc6ccccc6c45)ccc23)cc1 ; c1ccc(-c2cccc3c2sc2c(N(c4ccc5ccc6ccccc6c5c4)c4ccc(-c5cccc6ccccc56)c5oc6ccccc6c45)cccc23)cc1] 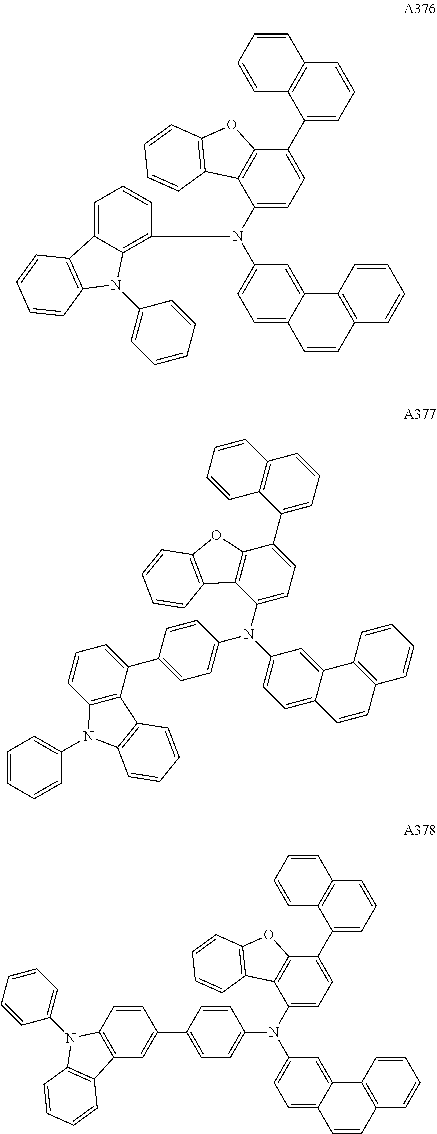 [SMILES: c1ccc(-n2c3ccccc3c3cccc(N(c4ccc5ccc6ccccc6c5c4)c4ccc(-c5cccc6ccccc56)c5oc6ccccc6c45)c32)cc1 ; c1ccc(-n2c3ccccc3c3c(-c4ccc(N(c5ccc6ccc7ccccc7c6c5)c5ccc(-c6cccc7ccccc67)c6oc7ccccc7c56)cc4)cccc32)cc1 ; c1ccc(-n2c3ccccc3c3cc(-c4ccc(N(c5ccc6ccc7ccccc7c6c5)c5ccc(-c6cccc7ccccc67)c6oc7ccccc7c56)cc4)ccc32)cc1] 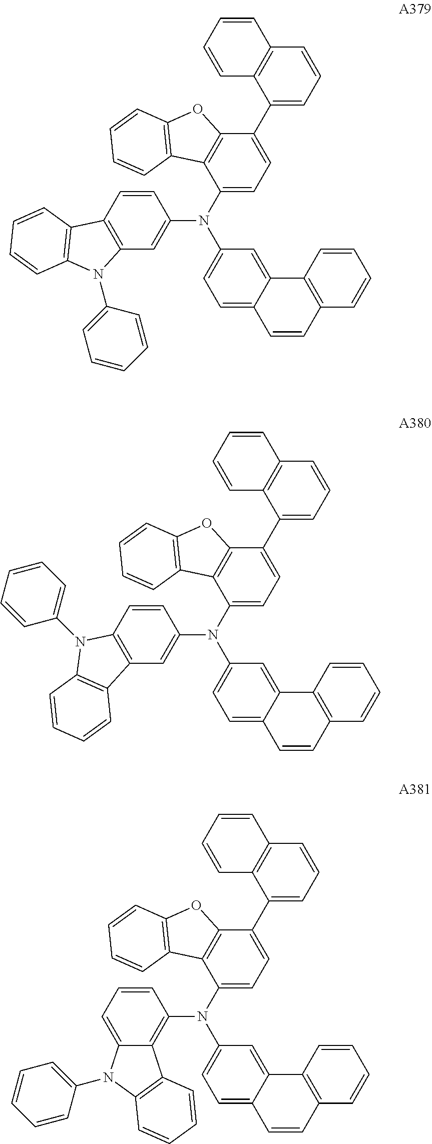 [SMILES: c1ccc(-n2c3ccccc3c3ccc(N(c4ccc5ccc6ccccc6c5c4)c4ccc(-c5cccc6ccccc56)c5oc6ccccc6c45)cc32)cc1 ; c1ccc(-n2c3ccccc3c3cc(N(c4ccc5ccc6ccccc6c5c4)c4ccc(-c5cccc6ccccc56)c5oc6ccccc6c45)ccc32)cc1 ; c1ccc(-n2c3ccccc3c3c(N(c4ccc5ccc6ccccc6c5c4)c4ccc(-c5cccc6ccccc56)c5oc6ccccc6c45)cccc32)cc1] 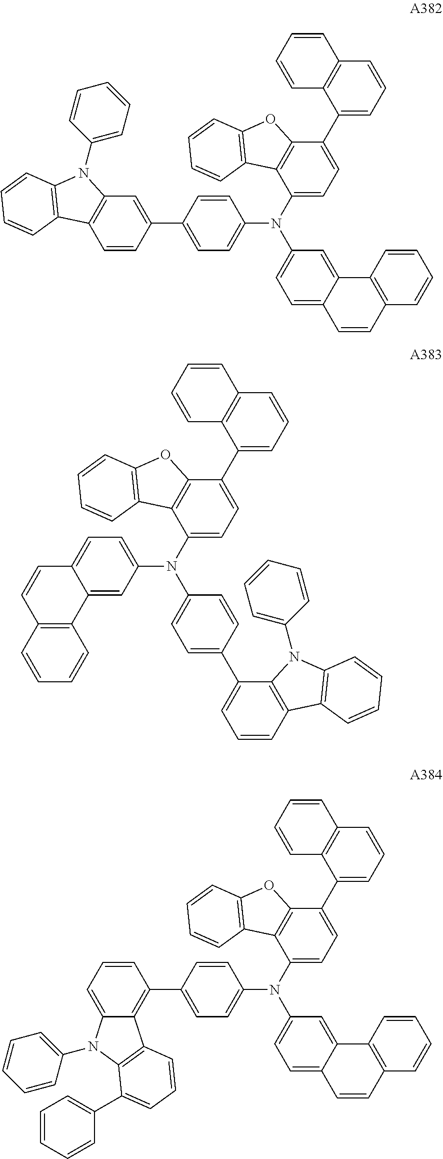 [SMILES: c1ccc(-n2c3ccccc3c3ccc(-c4ccc(N(c5ccc6ccc7ccccc7c6c5)c5ccc(-c6cccc7ccccc67)c6oc7ccccc7c56)cc4)cc32)cc1 ; c1ccc(-n2c3ccccc3c3cccc(-c4ccc(N(c5ccc6ccc7ccccc7c6c5)c5ccc(-c6cccc7ccccc67)c6oc7ccccc7c56)cc4)c32)cc1 ; c1ccc(-c2cccc3c4c(-c5ccc(N(c6ccc7ccc8ccccc8c7c6)c6ccc(-c7cccc8ccccc78)c7oc8ccccc8c67)cc5)cccc4n(-c4ccccc4)c23)cc1] 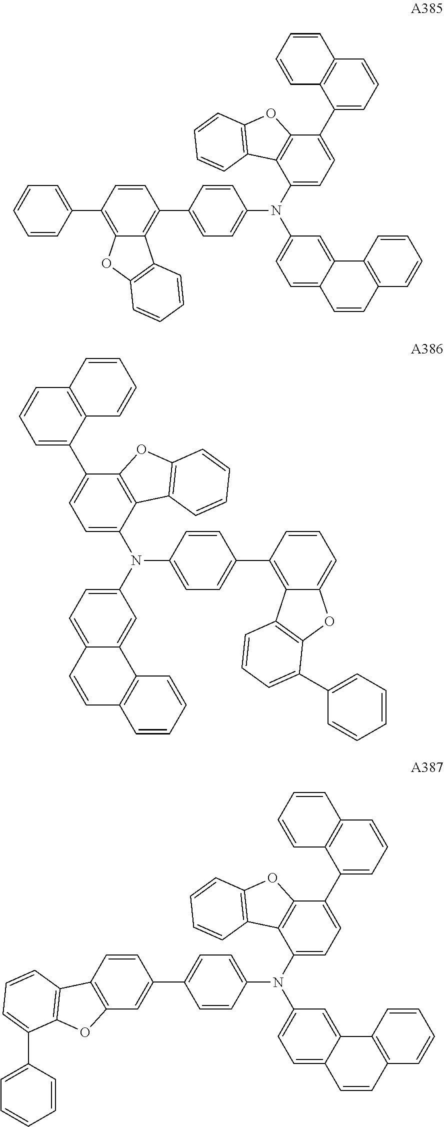 [SMILES: c1ccc(-c2ccc(-c3ccc(N(c4ccc5ccc6ccccc6c5c4)c4ccc(-c5cccc6ccccc56)c5oc6ccccc6c45)cc3)c3c2oc2ccccc23)cc1 ; c1ccc(-c2cccc3c2oc2cccc(-c4ccc(N(c5ccc6ccc7ccccc7c6c5)c5ccc(-c6cccc7ccccc67)c6oc7ccccc7c56)cc4)c23)cc1 ; c1ccc(-c2cccc3c2oc2cc(-c4ccc(N(c5ccc6ccc7ccccc7c6c5)c5ccc(-c6cccc7ccccc67)c6oc7ccccc7c56)cc4)ccc23)cc1] 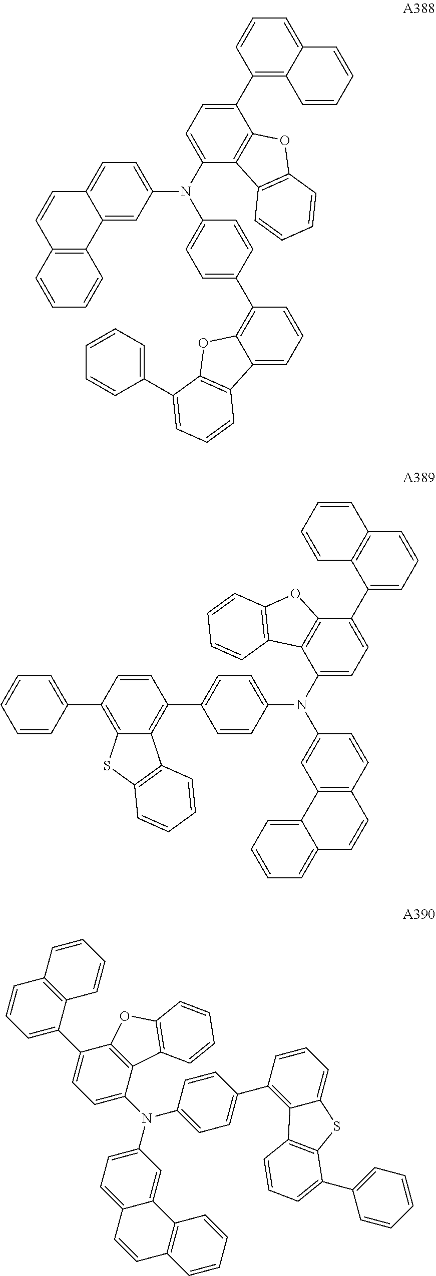 [SMILES: c1ccc(-c2cccc3c2oc2c(-c4ccc(N(c5ccc6ccc7ccccc7c6c5)c5ccc(-c6cccc7ccccc67)c6oc7ccccc7c56)cc4)cccc23)cc1 ; c1ccc(-c2ccc(-c3ccc(N(c4ccc5ccc6ccccc6c5c4)c4ccc(-c5cccc6ccccc56)c5oc6ccccc6c45)cc3)c3c2sc2ccccc23)cc1 ; c1ccc(-c2cccc3c2sc2cccc(-c4ccc(N(c5ccc6ccc7ccccc7c6c5)c5ccc(-c6cccc7ccccc67)c6oc7ccccc7c56)cc4)c23)cc1] 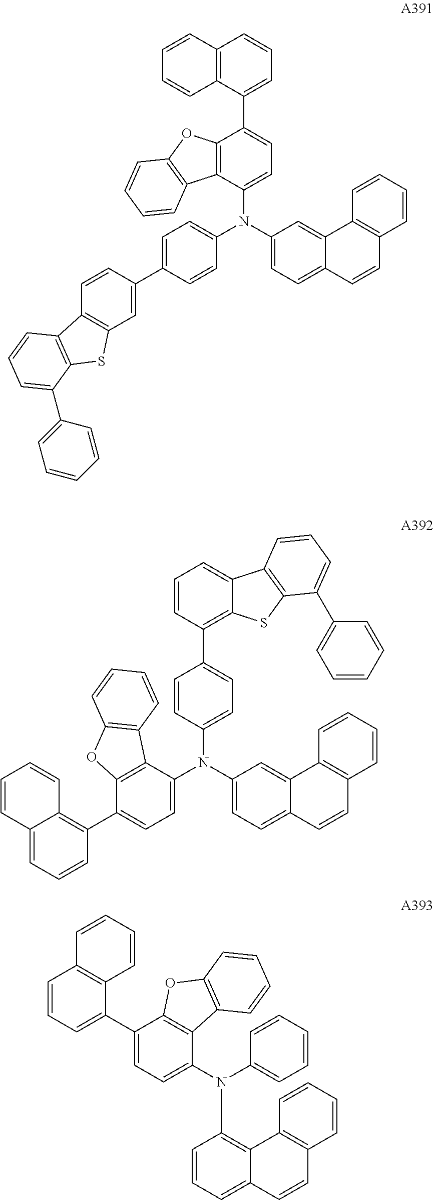 [SMILES: c1ccc(-c2cccc3c2sc2cc(-c4ccc(N(c5ccc6ccc7ccccc7c6c5)c5ccc(-c6cccc7ccccc67)c6oc7ccccc7c56)cc4)ccc23)cc1 ; c1ccc(-c2cccc3c2sc2c(-c4ccc(N(c5ccc6ccc7ccccc7c6c5)c5ccc(-c6cccc7ccccc67)c6oc7ccccc7c56)cc4)cccc23)cc1 ; c1ccc(N(c2cccc3ccc4ccccc4c23)c2ccc(-c3cccc4ccccc34)c3oc4ccccc4c23)cc1] 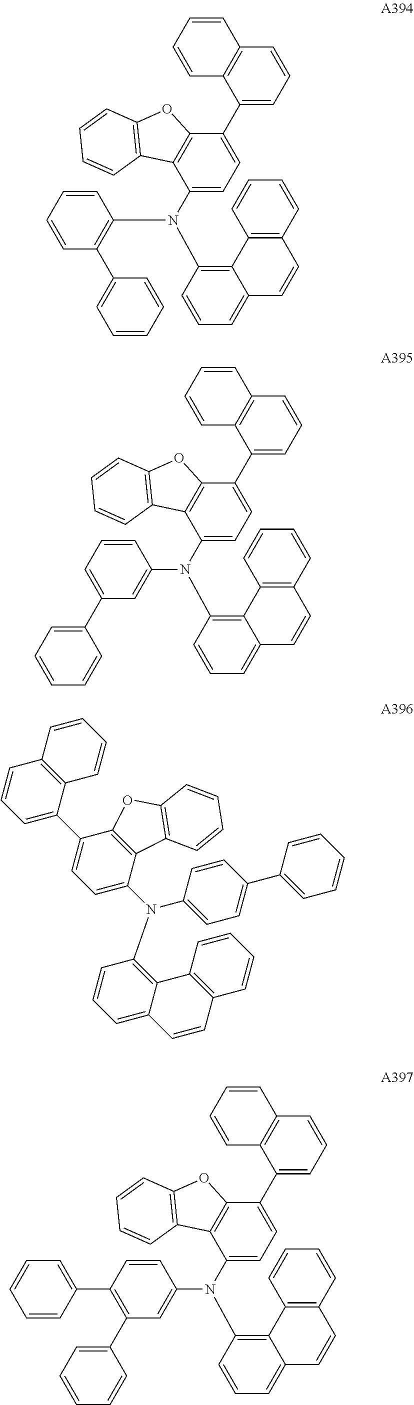 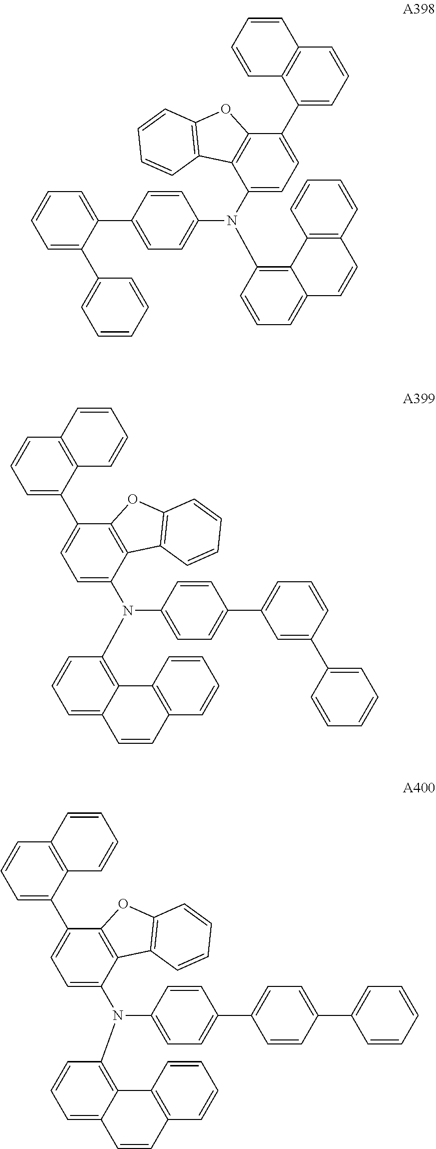 [SMILES: c1ccc(-c2ccccc2-c2ccc(N(c3cccc4ccc5ccccc5c34)c3ccc(-c4cccc5ccccc45)c4oc5ccccc5c34)cc2)cc1 ; c1ccc(-c2cccc(-c3ccc(N(c4cccc5ccc6ccccc6c45)c4ccc(-c5cccc6ccccc56)c5oc6ccccc6c45)cc3)c2)cc1 ; c1ccc(-c2ccc(-c3ccc(N(c4cccc5ccc6ccccc6c45)c4ccc(-c5cccc6ccccc56)c5oc6ccccc6c45)cc3)cc2)cc1] 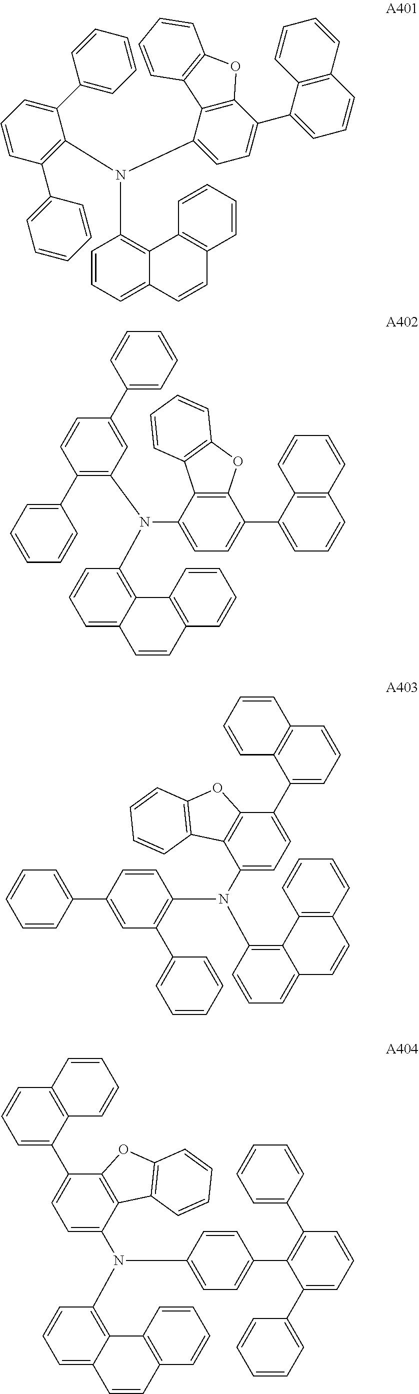 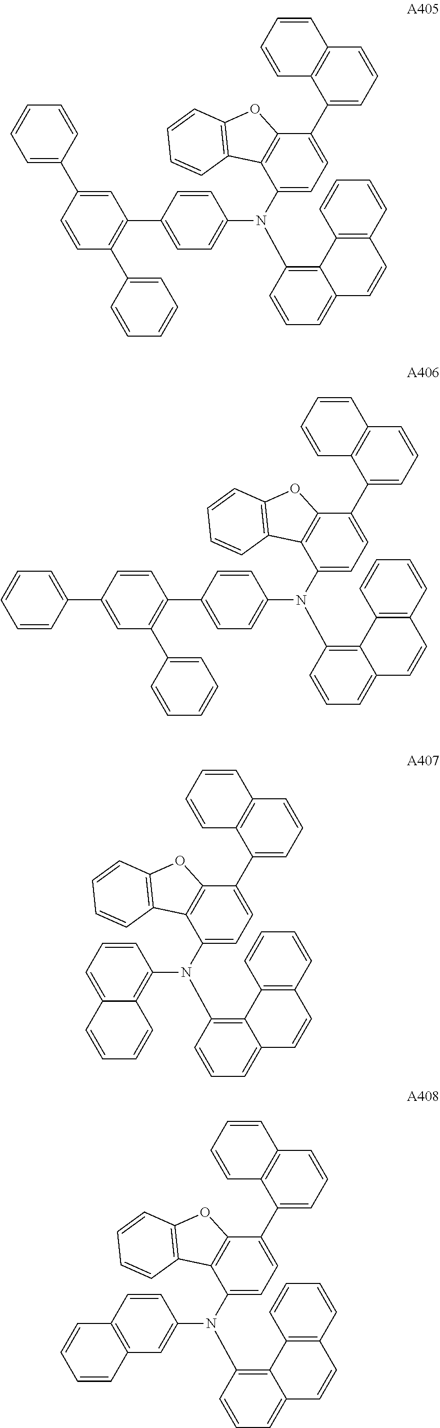 [SMILES: c1ccc(-c2ccc(-c3ccccc3)c(-c3ccc(N(c4cccc5ccc6ccccc6c45)c4ccc(-c5cccc6ccccc56)c5oc6ccccc6c45)cc3)c2)cc1 ; c1ccc(-c2ccc(-c3ccc(N(c4cccc5ccc6ccccc6c45)c4ccc(-c5cccc6ccccc56)c5oc6ccccc6c45)cc3)c(-c3ccccc3)c2)cc1 ; c1ccc2c(-c3ccc(N(c4cccc5ccccc45)c4cccc5ccc6ccccc6c45)c4c3oc3ccccc34)cccc2c1 ; c1ccc2cc(N(c3cccc4ccc5ccccc5c34)c3ccc(-c4cccc5ccccc45)c4oc5ccccc5c34)ccc2c1] 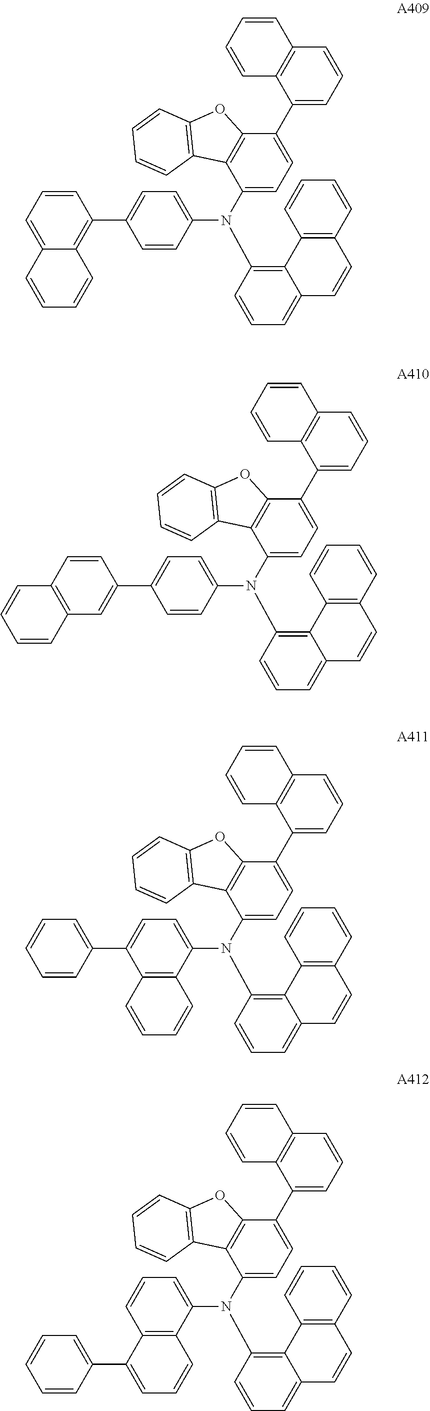 [SMILES: c1ccc2c(-c3ccc(N(c4cccc5ccc6ccccc6c45)c4ccc(-c5cccc6ccccc56)c5oc6ccccc6c45)cc3)cccc2c1 ; c1ccc2cc(-c3ccc(N(c4cccc5ccc6ccccc6c45)c4ccc(-c5cccc6ccccc56)c5oc6ccccc6c45)cc3)ccc2c1 ; c1ccc(-c2ccc(N(c3cccc4ccc5ccccc5c34)c3ccc(-c4cccc5ccccc45)c4oc5ccccc5c34)c3ccccc23)cc1 ; c1ccc(-c2cccc3c(N(c4cccc5ccc6ccccc6c45)c4ccc(-c5cccc6ccccc56)c5oc6ccccc6c45)cccc23)cc1] 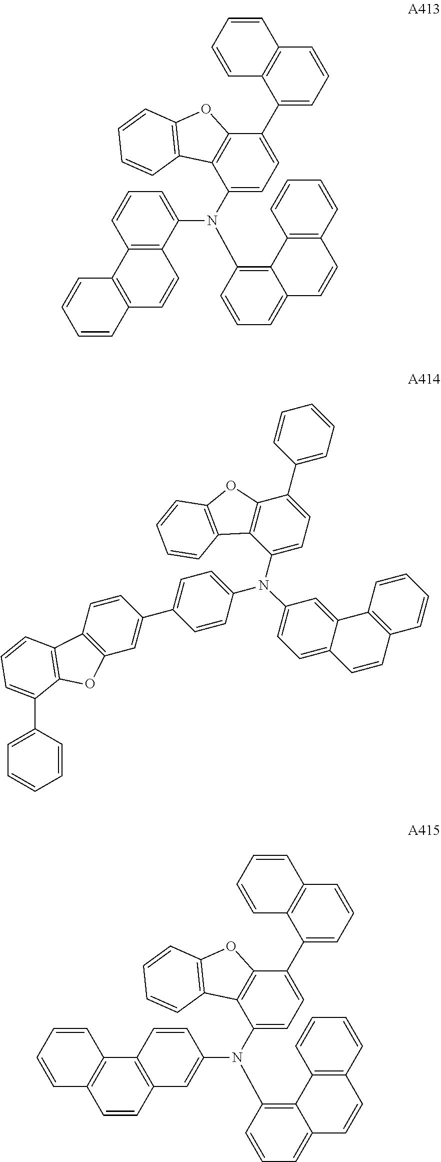 [SMILES: c1ccc2c(-c3ccc(N(c4cccc5c4ccc4ccccc45)c4cccc5ccc6ccccc6c45)c4c3oc3ccccc34)cccc2c1 ; c1ccc(-c2cccc3c2oc2cc(-c4ccc(N(c5ccc6ccc7ccccc7c6c5)c5ccc(-c6ccccc6)c6oc7ccccc7c56)cc4)ccc23)cc1 ; c1ccc2c(-c3ccc(N(c4ccc5c(ccc6ccccc65)c4)c4cccc5ccc6ccccc6c45)c4c3oc3ccccc34)cccc2c1] 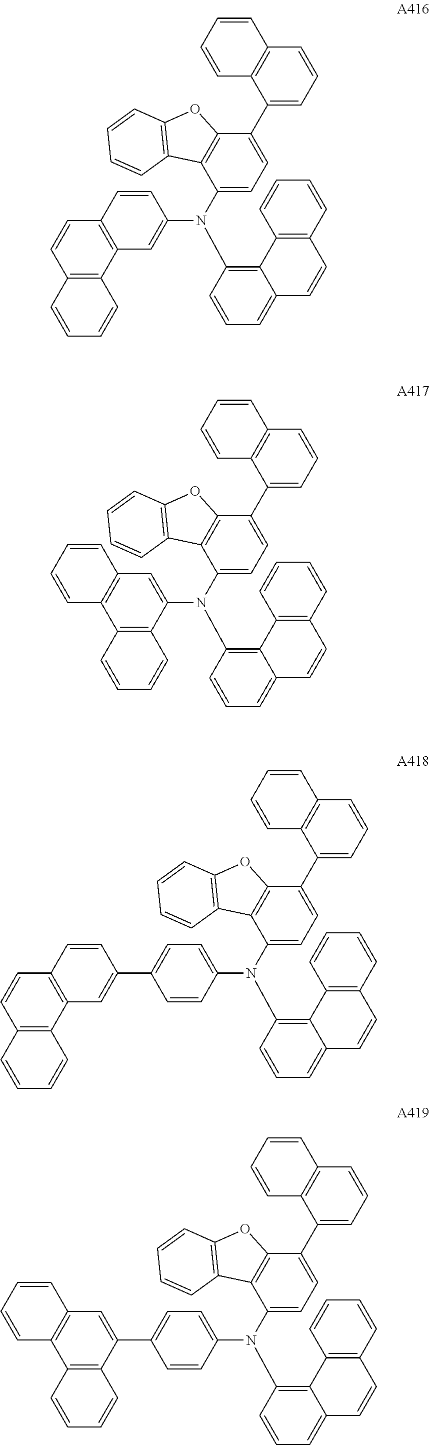 [SMILES: c1ccc2c(-c3ccc(N(c4ccc5ccc6ccccc6c5c4)c4cccc5ccc6ccccc6c45)c4c3oc3ccccc34)cccc2c1 ; c1ccc2c(-c3ccc(N(c4cc5ccccc5c5ccccc45)c4cccc5ccc6ccccc6c45)c4c3oc3ccccc34)cccc2c1 ; c1ccc2c(-c3ccc(N(c4ccc(-c5ccc6ccc7ccccc7c6c5)cc4)c4cccc5ccc6ccccc6c45)c4c3oc3ccccc34)cccc2c1 ; c1ccc2c(-c3ccc(N(c4ccc(-c5cc6ccccc6c6ccccc56)cc4)c4cccc5ccc6ccccc6c45)c4c3oc3ccccc34)cccc2c1] 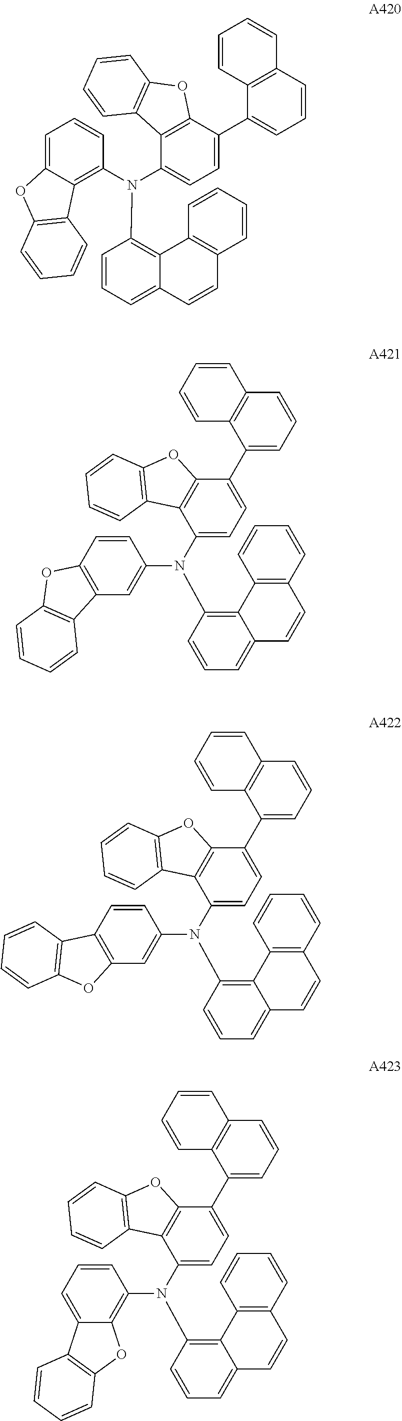 [SMILES: c1ccc2c(-c3ccc(N(c4cccc5ccc6ccccc6c45)c4cccc5oc6ccccc6c45)c4c3oc3ccccc34)cccc2c1 ; c1ccc2c(-c3ccc(N(c4ccc5oc6ccccc6c5c4)c4cccc5ccc6ccccc6c45)c4c3oc3ccccc34)cccc2c1 ; c1ccc2c(-c3ccc(N(c4ccc5c(c4)oc4ccccc45)c4cccc5ccc6ccccc6c45)c4c3oc3ccccc34)cccc2c1 ; c1ccc2c(-c3ccc(N(c4cccc5c4oc4ccccc45)c4cccc5ccc6ccccc6c45)c4c3oc3ccccc34)cccc2c1] 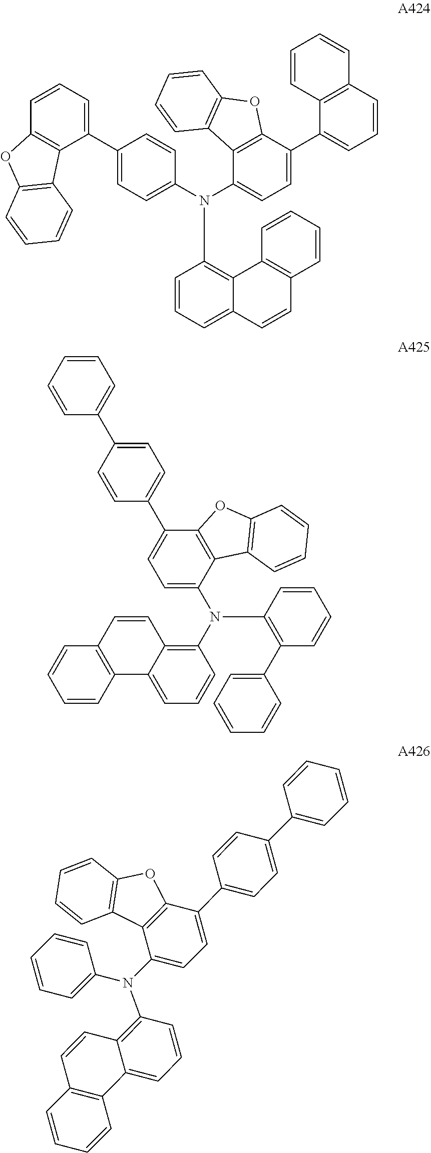 [SMILES: c1ccc2c(-c3ccc(N(c4ccc(-c5cccc6oc7ccccc7c56)cc4)c4cccc5ccc6ccccc6c45)c4c3oc3ccccc34)cccc2c1 ; c1ccc(-c2ccc(-c3ccc(N(c4ccccc4-c4ccccc4)c4cccc5c4ccc4ccccc45)c4c3oc3ccccc34)cc2)cc1 ; c1ccc(-c2ccc(-c3ccc(N(c4ccccc4)c4cccc5c4ccc4ccccc45)c4c3oc3ccccc34)cc2)cc1] 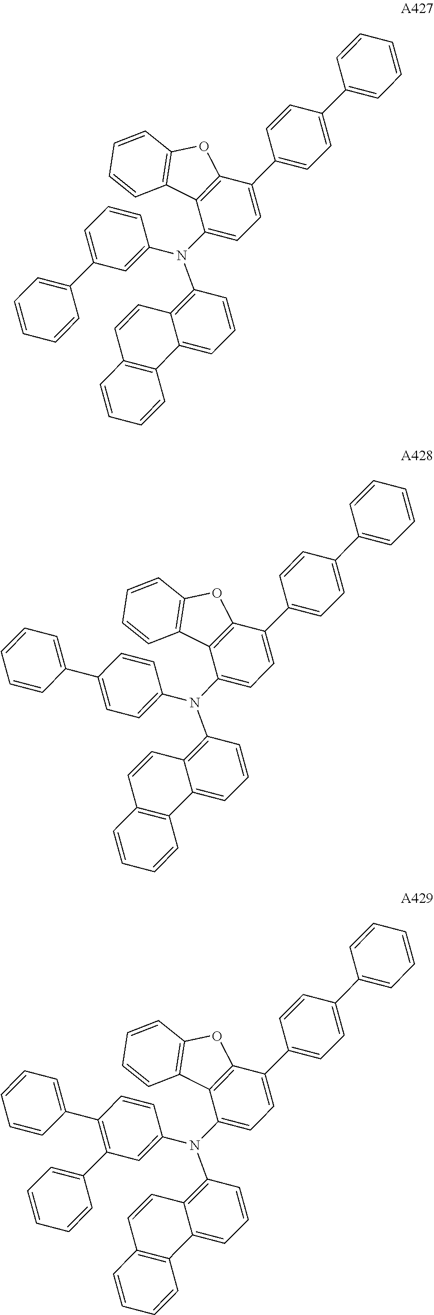 [SMILES: c1ccc(-c2ccc(-c3ccc(N(c4cccc(-c5ccccc5)c4)c4cccc5c4ccc4ccccc45)c4c3oc3ccccc34)cc2)cc1 ; c1ccc(-c2ccc(-c3ccc(N(c4ccc(-c5ccccc5)cc4)c4cccc5c4ccc4ccccc45)c4c3oc3ccccc34)cc2)cc1 ; c1ccc(-c2ccc(-c3ccc(N(c4ccc(-c5ccccc5)c(-c5ccccc5)c4)c4cccc5c4ccc4ccccc45)c4c3oc3ccccc34)cc2)cc1] 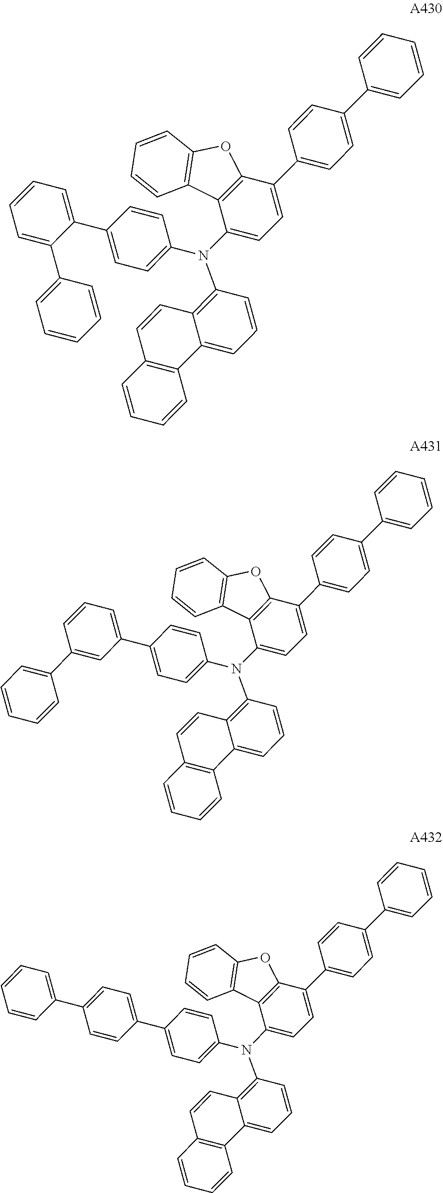 [SMILES: c1ccc(-c2ccc(-c3ccc(N(c4ccc(-c5ccccc5-c5ccccc5)cc4)c4cccc5c4ccc4ccccc45)c4c3oc3ccccc34)cc2)cc1 ; c1ccc(-c2ccc(-c3ccc(N(c4ccc(-c5cccc(-c6ccccc6)c5)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccc(-c6ccccc6)cc5)c5oc6ccccc6c45)cc3)cc2)cc1] 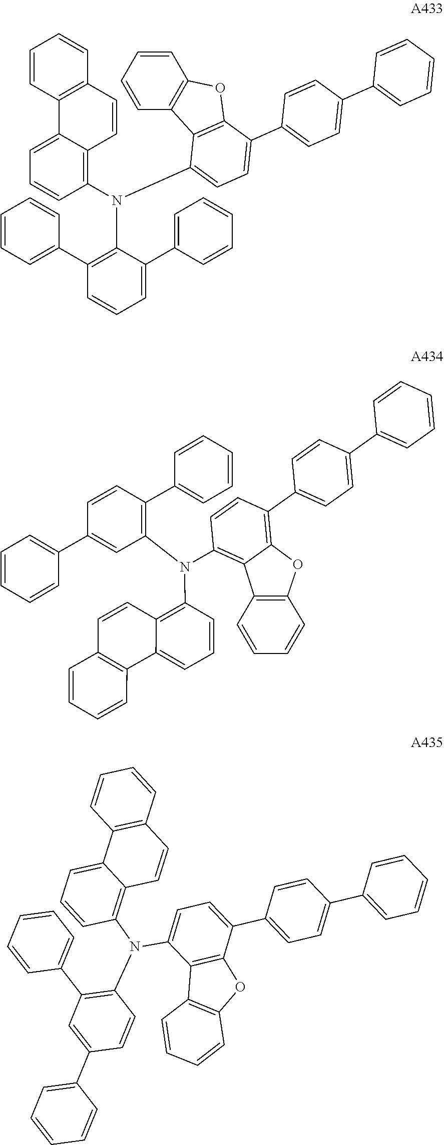 [SMILES: c1ccc(-c2ccc(-c3ccc(N(c4c(-c5ccccc5)cccc4-c4ccccc4)c4cccc5c4ccc4ccccc45)c4c3oc3ccccc34)cc2)cc1 ; c1ccc(-c2ccc(-c3ccc(N(c4cc(-c5ccccc5)ccc4-c4ccccc4)c4cccc5c4ccc4ccccc45)c4c3oc3ccccc34)cc2)cc1 ; c1ccc(-c2ccc(-c3ccc(N(c4ccc(-c5ccccc5)cc4-c4ccccc4)c4cccc5c4ccc4ccccc45)c4c3oc3ccccc34)cc2)cc1] 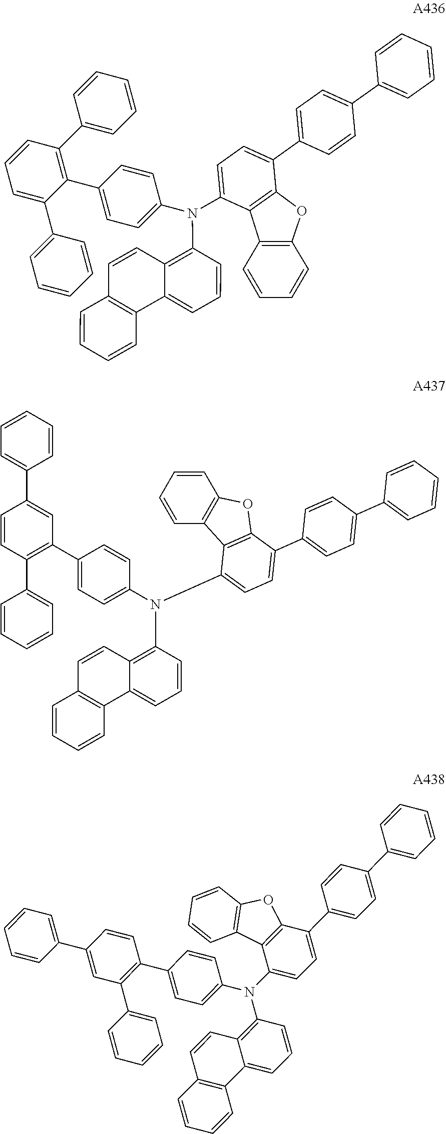 [SMILES: c1ccc(-c2ccc(-c3ccc(N(c4ccc(-c5c(-c6ccccc6)cccc5-c5ccccc5)cc4)c4cccc5c4ccc4ccccc45)c4c3oc3ccccc34)cc2)cc1 ; c1ccc(-c2ccc(-c3ccc(N(c4ccc(-c5cc(-c6ccccc6)ccc5-c5ccccc5)cc4)c4cccc5c4ccc4ccccc45)c4c3oc3ccccc34)cc2)cc1 ; c1ccc(-c2ccc(-c3ccc(N(c4ccc(-c5ccc(-c6ccccc6)cc5-c5ccccc5)cc4)c4cccc5c4ccc4ccccc45)c4c3oc3ccccc34)cc2)cc1] 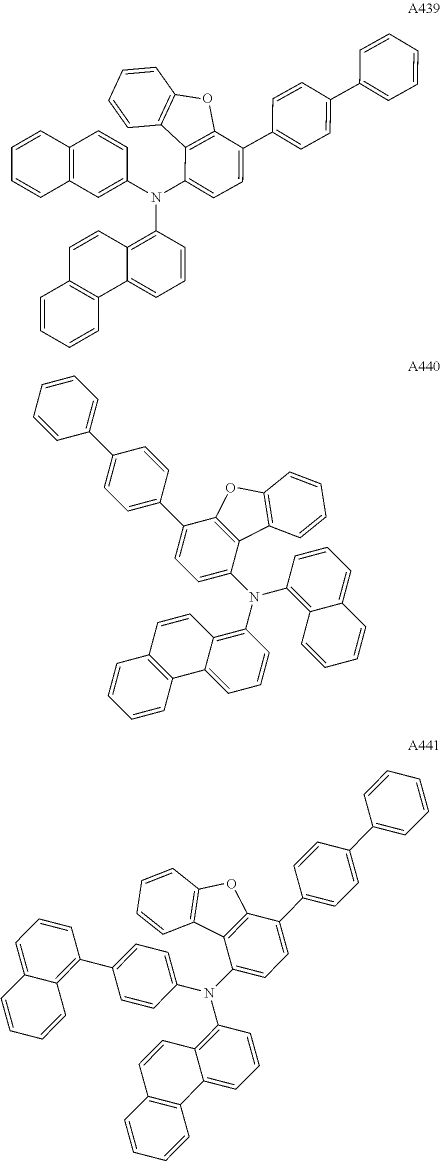 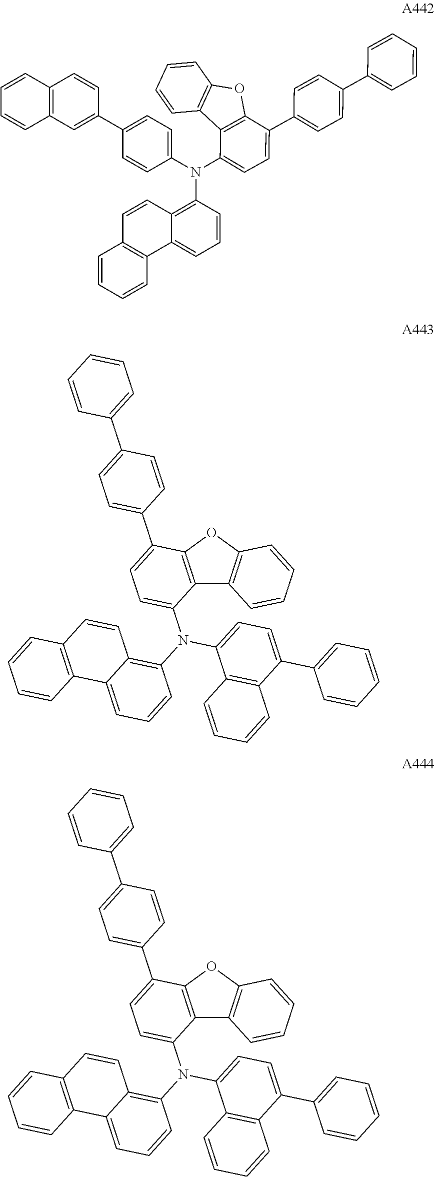 [SMILES: c1ccc(-c2ccc(-c3ccc(N(c4ccc(-c5ccc6ccccc6c5)cc4)c4cccc5c4ccc4ccccc45)c4c3oc3ccccc34)cc2)cc1 ; c1ccc(-c2ccc(-c3ccc(N(c4ccc(-c5ccccc5)c5ccccc45)c4cccc5c4ccc4ccccc45)c4c3oc3ccccc34)cc2)cc1 ; c1ccc(-c2ccc(-c3ccc(N(c4ccc(-c5ccccc5)c5ccccc45)c4cccc5c4ccc4ccccc45)c4c3oc3ccccc34)cc2)cc1] 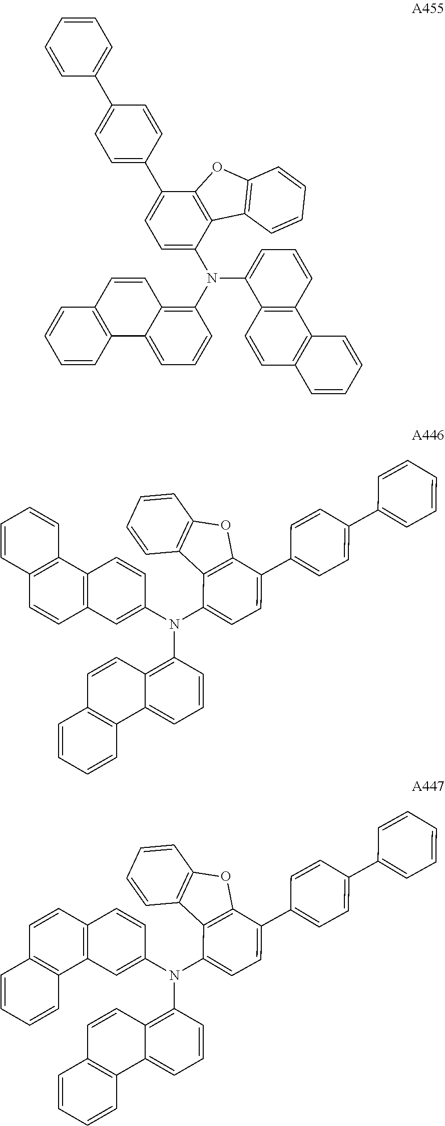 [SMILES: c1ccc(-c2ccc(-c3ccc(N(c4cccc5c4ccc4ccccc45)c4cccc5c4ccc4ccccc45)c4c3oc3ccccc34)cc2)cc1 ; c1ccc(-c2ccc(-c3ccc(N(c4ccc5c(ccc6ccccc65)c4)c4cccc5c4ccc4ccccc45)c4c3oc3ccccc34)cc2)cc1 ; c1ccc(-c2ccc(-c3ccc(N(c4ccc5ccc6ccccc6c5c4)c4cccc5c4ccc4ccccc45)c4c3oc3ccccc34)cc2)cc1] 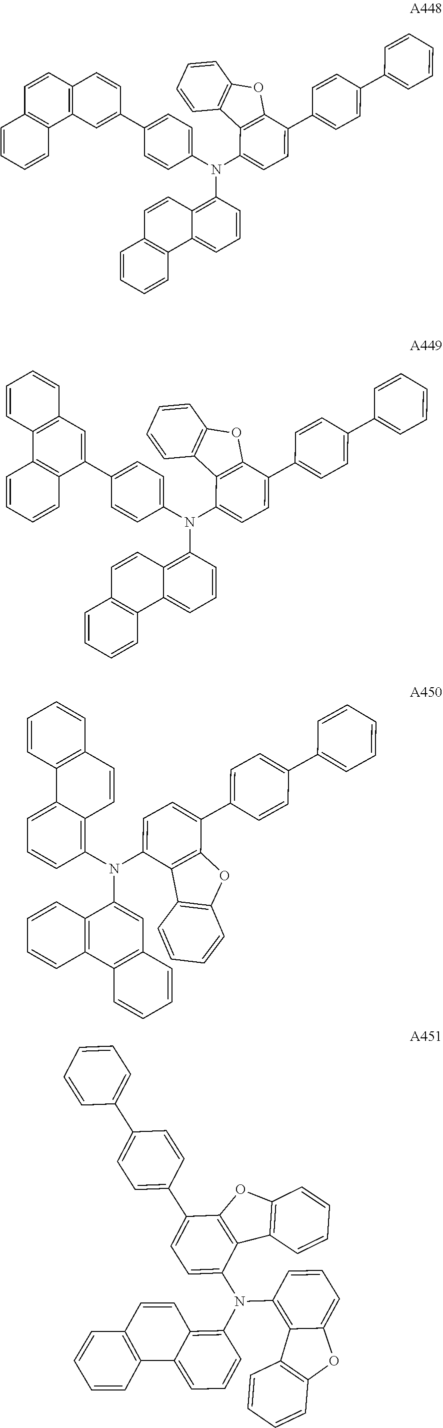 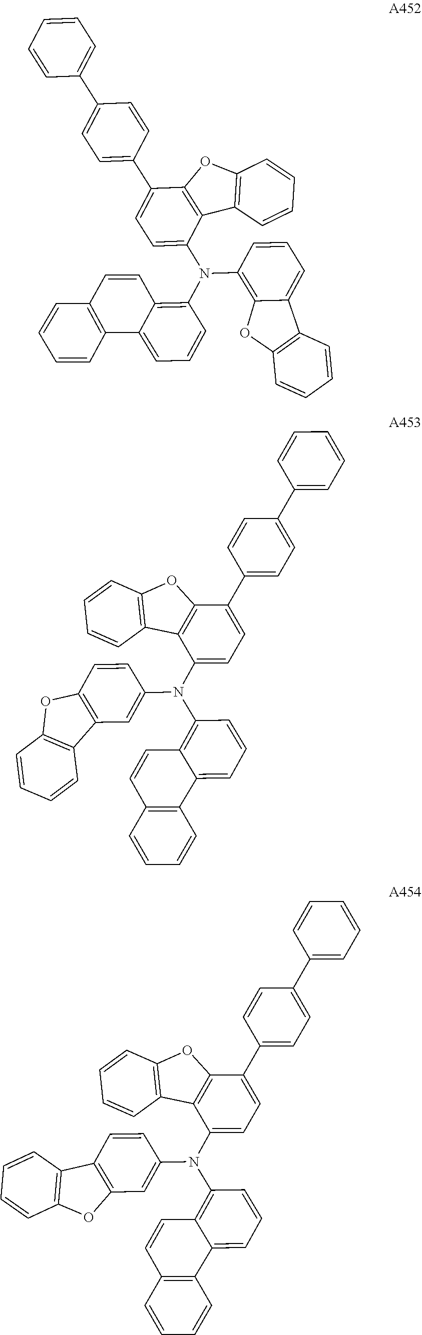 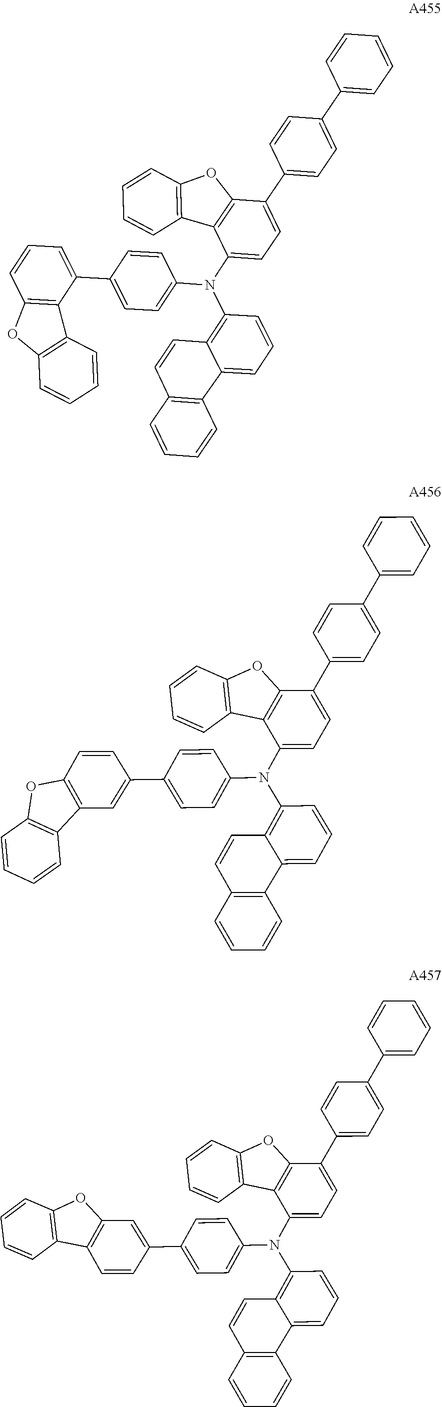 [SMILES: c1ccc(-c2ccc(-c3ccc(N(c4ccc(-c5cccc6oc7ccccc7c56)cc4)c4cccc5c4ccc4ccccc45)c4c3oc3ccccc34)cc2)cc1 ; c1ccc(-c2ccc(-c3ccc(N(c4ccc(-c5ccc6oc7ccccc7c6c5)cc4)c4cccc5c4ccc4ccccc45)c4c3oc3ccccc34)cc2)cc1 ; c1ccc(-c2ccc(-c3ccc(N(c4ccc(-c5ccc6c(c5)oc5ccccc56)cc4)c4cccc5c4ccc4ccccc45)c4c3oc3ccccc34)cc2)cc1] 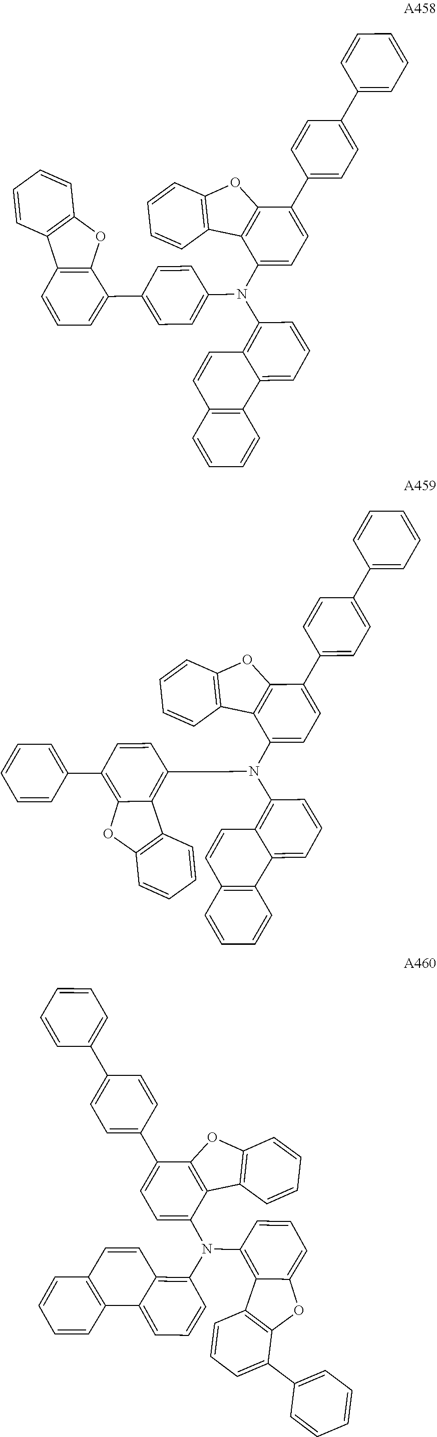 [SMILES: c1ccc(-c2ccc(-c3ccc(N(c4ccc(-c5cccc6c5oc5ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccccc5)c5oc6ccccc6c45)c4c3oc3ccccc34)cc2)cc1 ; c1ccc(-c2ccc(-c3ccc(N(c4cccc5c4ccc4ccccc45)c4cccc5oc6c(-c7ccccc7)cccc6c45)c4c3oc3ccccc34)cc2)cc1] 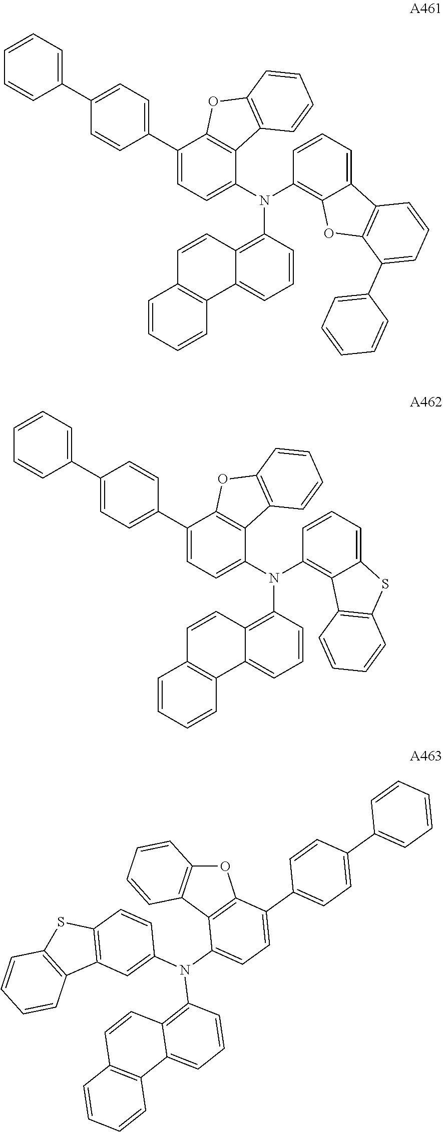 [SMILES: c1ccc(-c2ccc(-c3ccc(N(c4cccc5c4ccc4ccccc45)c4cccc5c4oc4c(-c6ccccc6)cccc45)c4c3oc3ccccc34)cc2)cc1 ; c1ccc(-c2ccc(-c3ccc(N(c4cccc5c4ccc4ccccc45)c4cccc5sc6ccccc6c45)c4c3oc3ccccc34)cc2)cc1 ; c1ccc(-c2ccc(-c3ccc(N(c4ccc5sc6ccccc6c5c4)c4cccc5c4ccc4ccccc45)c4c3oc3ccccc34)cc2)cc1] 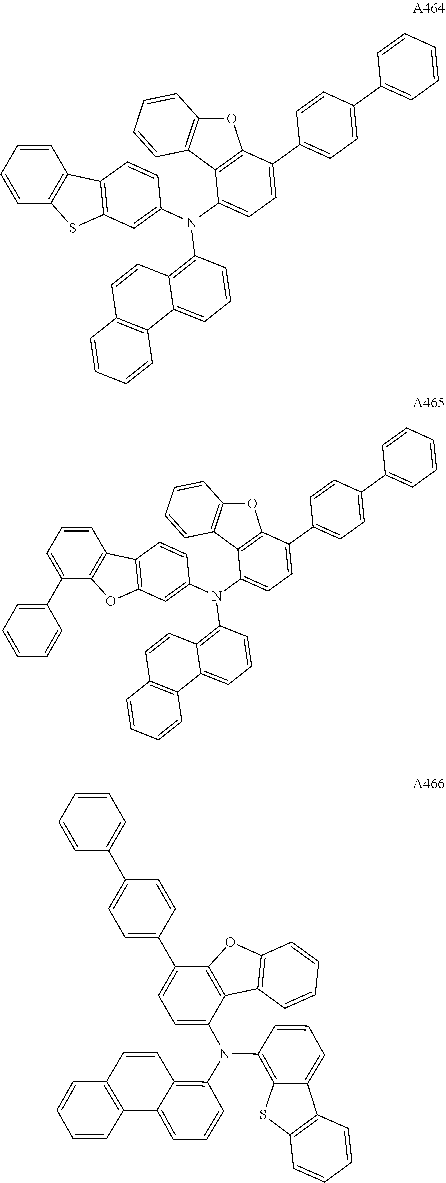 [SMILES: c1ccc(-c2ccc(-c3ccc(N(c4ccc5c(c4)sc4ccccc45)c4cccc5c4ccc4ccccc45)c4c3oc3ccccc34)cc2)cc1 ; c1ccc(-c2ccc(-c3ccc(N(c4ccc5c(c4)oc4c(-c6ccccc6)cccc45)c4cccc5c4ccc4ccccc45)c4c3oc3ccccc34)cc2)cc1 ; c1ccc(-c2ccc(-c3ccc(N(c4cccc5c4ccc4ccccc45)c4cccc5c4sc4ccccc45)c4c3oc3ccccc34)cc2)cc1] 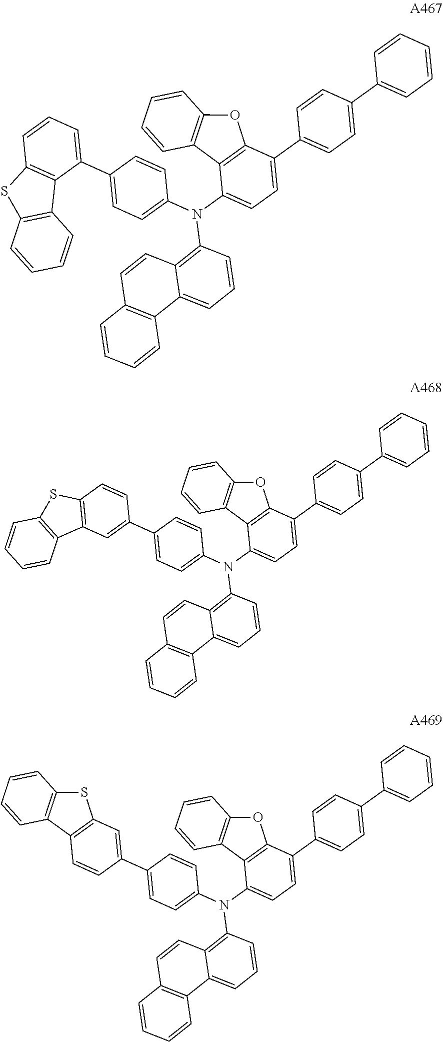 [SMILES: c1ccc(-c2ccc(-c3ccc(N(c4ccc(-c5cccc6sc7ccccc7c56)cc4)c4cccc5c4ccc4ccccc45)c4c3oc3ccccc34)cc2)cc1 ; c1ccc(-c2ccc(-c3ccc(N(c4ccc(-c5ccc6sc7ccccc7c6c5)cc4)c4cccc5c4ccc4ccccc45)c4c3oc3ccccc34)cc2)cc1 ; c1ccc(-c2ccc(-c3ccc(N(c4ccc(-c5ccc6c(c5)sc5ccccc56)cc4)c4cccc5c4ccc4ccccc45)c4c3oc3ccccc34)cc2)cc1] 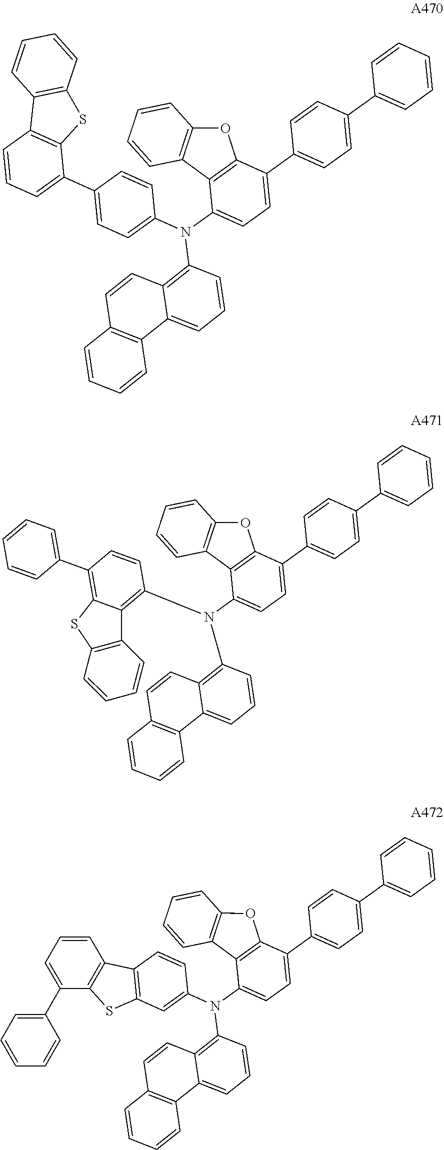 [SMILES: c1ccc(-c2ccc(-c3ccc(N(c4ccc(-c5cccc6c5sc5ccccc56)cc4)c4cccc5c4ccc4ccccc45)c4c3oc3ccccc34)cc2)cc1 ; c1ccc(-c2ccc(-c3ccc(N(c4cccc5c4ccc4ccccc45)c4ccc(-c5ccccc5)c5sc6ccccc6c45)c4c3oc3ccccc34)cc2)cc1 ; c1ccc(-c2ccc(-c3ccc(N(c4ccc5c(c4)sc4c(-c6ccccc6)cccc45)c4cccc5c4ccc4ccccc45)c4c3oc3ccccc34)cc2)cc1] 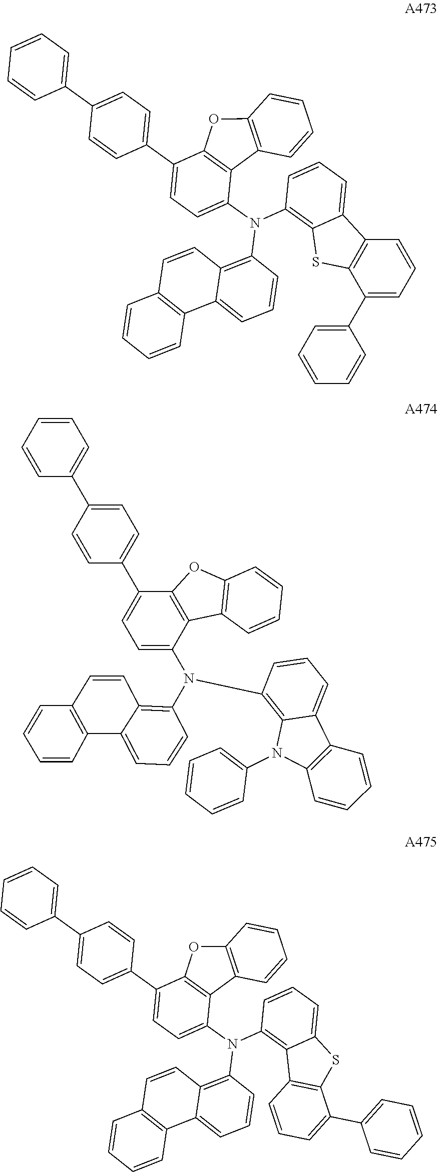 [SMILES: c1ccc(-c2ccc(-c3ccc(N(c4cccc5c4ccc4ccccc45)c4cccc5c4sc4c(-c6ccccc6)cccc45)c4c3oc3ccccc34)cc2)cc1 ; c1ccc(-c2ccc(-c3ccc(N(c4cccc5c4ccc4ccccc45)c4cccc5c6ccccc6n(-c6ccccc6)c45)c4c3oc3ccccc34)cc2)cc1 ; c1ccc(-c2ccc(-c3ccc(N(c4cccc5c4ccc4ccccc45)c4cccc5sc6c(-c7ccccc7)cccc6c45)c4c3oc3ccccc34)cc2)cc1] 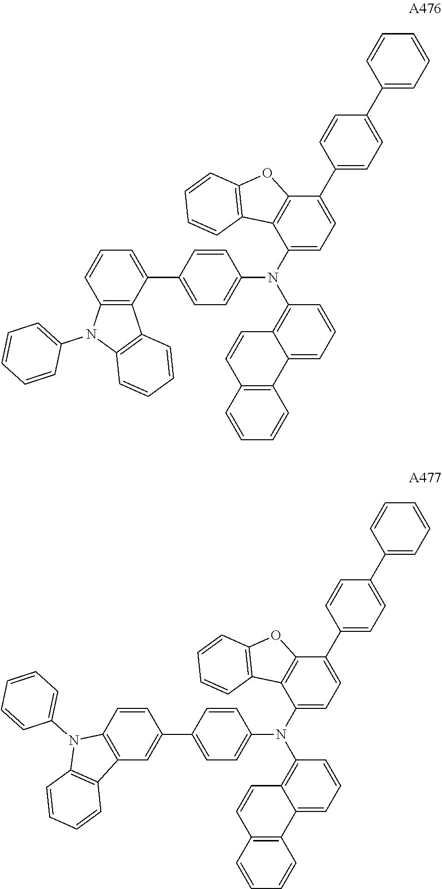 [SMILES: c1ccc(-c2ccc(-c3ccc(N(c4ccc(-c5cccc6c5c5ccccc5n6-c5ccccc5)cc4)c4cccc5c4ccc4ccccc45)c4c3oc3ccccc34)cc2)cc1 ; c1ccc(-c2ccc(-c3ccc(N(c4ccc(-c5ccc6c(c5)c5ccccc5n6-c5ccccc5)cc4)c4cccc5c4ccc4ccccc45)c4c3oc3ccccc34)cc2)cc1] 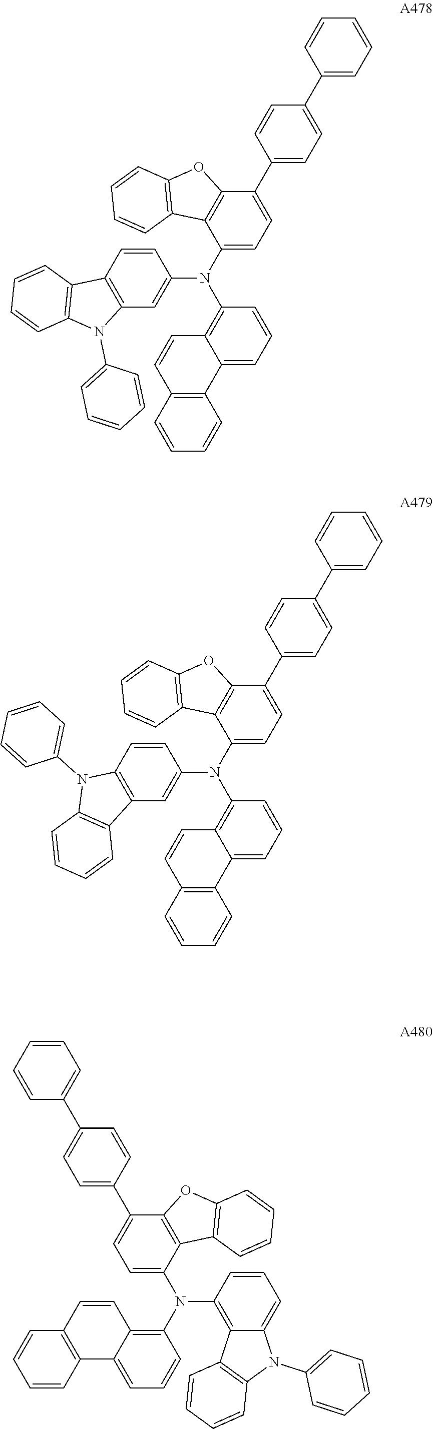 [SMILES: c1ccc(-c2ccc(-c3ccc(N(c4ccc5c6ccccc6n(-c6ccccc6)c5c4)c4cccc5c4ccc4ccccc45)c4c3oc3ccccc34)cc2)cc1 ; c1ccc(-c2ccc(-c3ccc(N(c4ccc5c(c4)c4ccccc4n5-c4ccccc4)c4cccc5c4ccc4ccccc45)c4c3oc3ccccc34)cc2)cc1 ; c1ccc(-c2ccc(-c3ccc(N(c4cccc5c4ccc4ccccc45)c4cccc5c4c4ccccc4n5-c4ccccc4)c4c3oc3ccccc34)cc2)cc1] 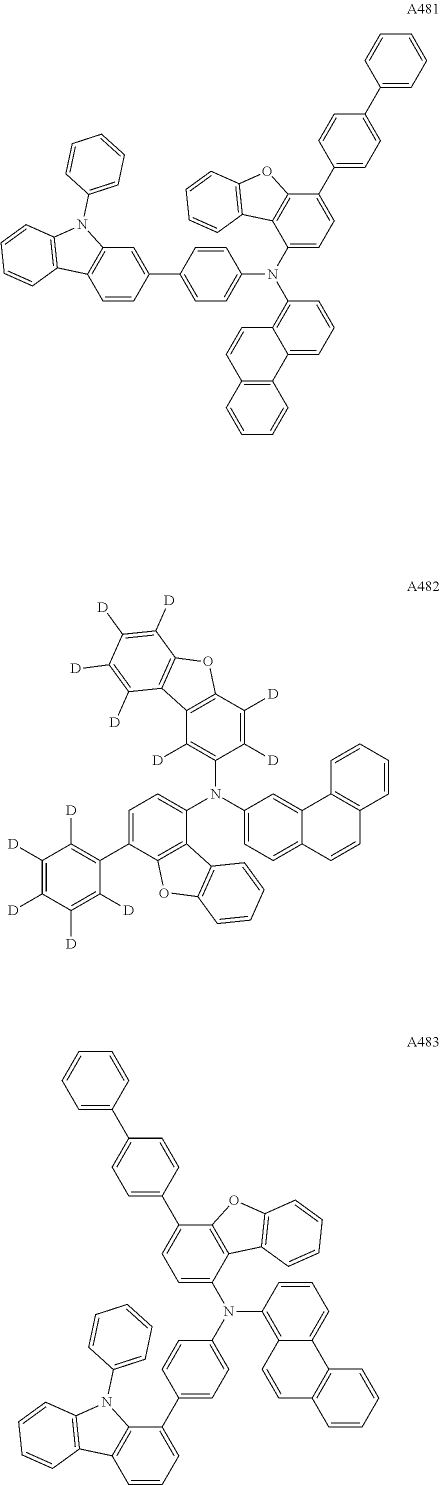 [SMILES: c1ccc(-c2ccc(-c3ccc(N(c4ccc(-c5ccc6c7ccccc7n(-c7ccccc7)c6c5)cc4)c4cccc5c4ccc4ccccc45)c4c3oc3ccccc34)cc2)cc1 ; [2H]c1c([2H])c([2H])c(-c2ccc(N(c3ccc4ccc5ccccc5c4c3)c3c([2H])c([2H])c4oc5c([2H])c([2H])c([2H])c([2H])c5c4c3[2H])c3c2oc2ccccc23)c([2H])c1[2H] ; c1ccc(-c2ccc(-c3ccc(N(c4ccc(-c5cccc6c7ccccc7n(-c7ccccc7)c56)cc4)c4cccc5c4ccc4ccccc45)c4c3oc3ccccc34)cc2)cc1] 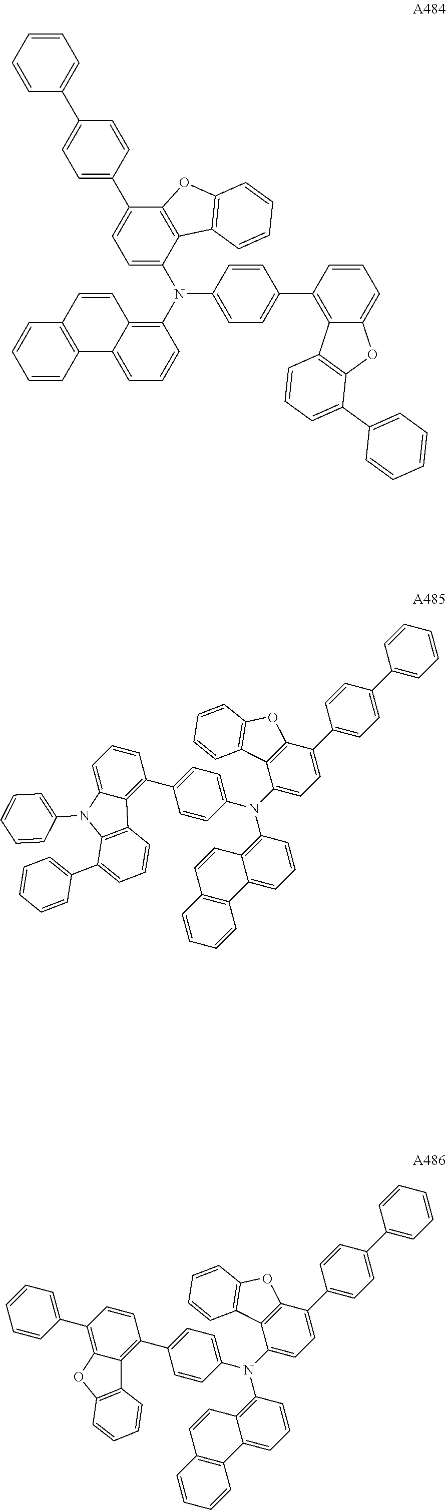 [SMILES: c1ccc(-c2ccc(-c3ccc(N(c4ccc(-c5cccc6oc7c(-c8ccccc8)cccc7c56)cc4)c4cccc5c4ccc4ccccc45)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4cccc5c4ccc4ccccc45)c4c3oc3ccccc34)cc2)cc1 ; c1ccc(-c2ccc(-c3ccc(N(c4ccc(-c5ccc(-c6ccccc6)c6oc7ccccc7c56)cc4)c4cccc5c4ccc4ccccc45)c4c3oc3ccccc34)cc2)cc1] 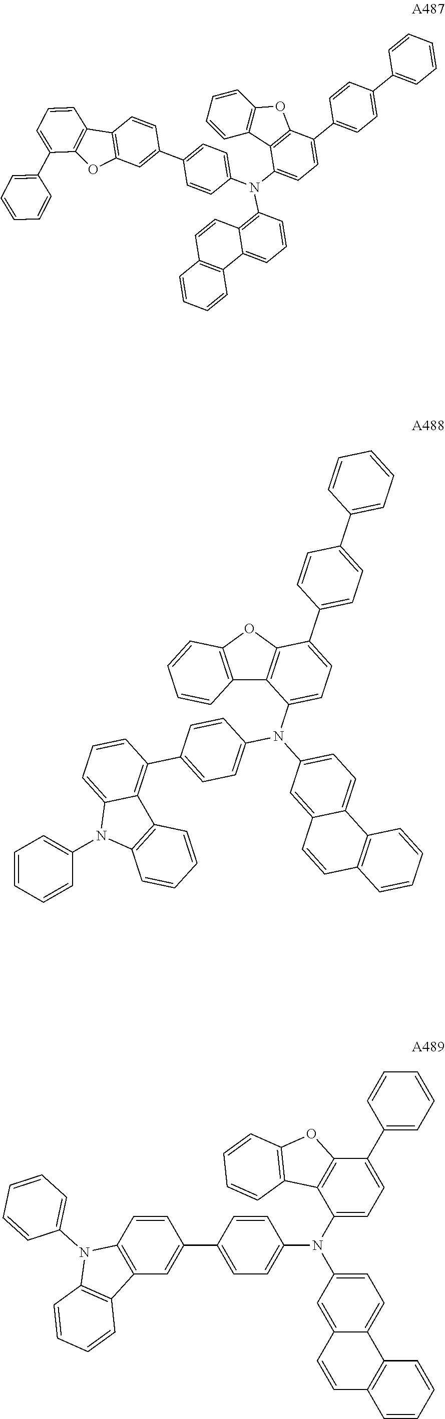 [SMILES: c1ccc(-c2ccc(-c3ccc(N(c4ccc(-c5ccc6c(c5)oc5c(-c7ccccc7)cccc56)cc4)c4cccc5c4ccc4ccccc45)c4c3oc3ccccc34)cc2)cc1 ; c1ccc(-c2ccc(-c3ccc(N(c4ccc(-c5cccc6c5c5ccccc5n6-c5ccccc5)cc4)c4ccc5c(ccc6ccccc65)c4)c4c3oc3ccccc34)cc2)cc1 ; c1ccc(-c2ccc(N(c3ccc(-c4ccc5c(c4)c4ccccc4n5-c4ccccc4)cc3)c3ccc4c(ccc5ccccc54)c3)c3c2oc2ccccc23)cc1] 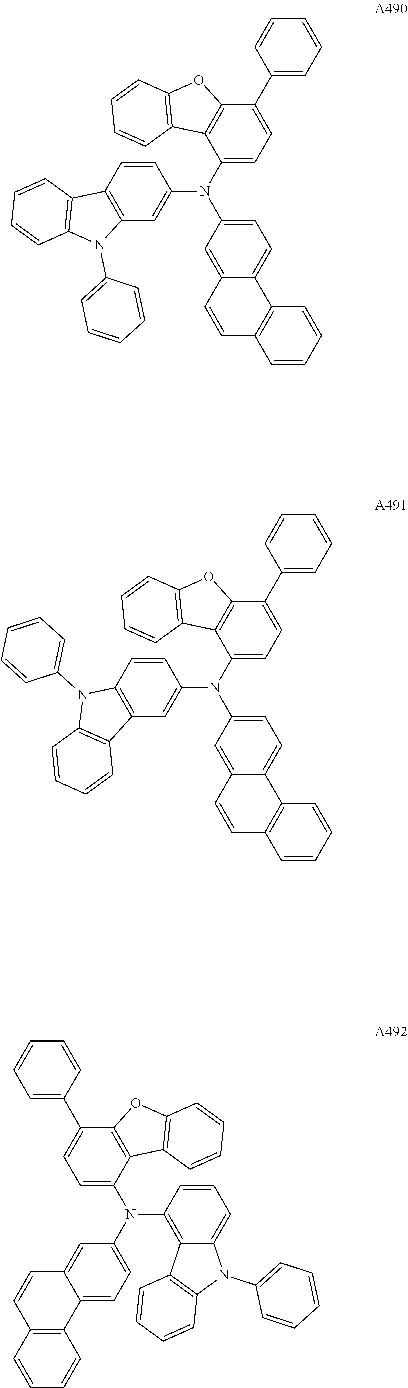 [SMILES: c1ccc(-c2ccc(N(c3ccc4c(ccc5ccccc54)c3)c3ccc4c5ccccc5n(-c5ccccc5)c4c3)c3c2oc2ccccc23)cc1 ; c1ccc(-c2ccc(N(c3ccc4c(ccc5ccccc54)c3)c3ccc4c(c3)c3ccccc3n4-c3ccccc3)c3c2oc2ccccc23)cc1 ; c1ccc(-c2ccc(N(c3ccc4c(ccc5ccccc54)c3)c3cccc4c3c3ccccc3n4-c3ccccc3)c3c2oc2ccccc23)cc1] 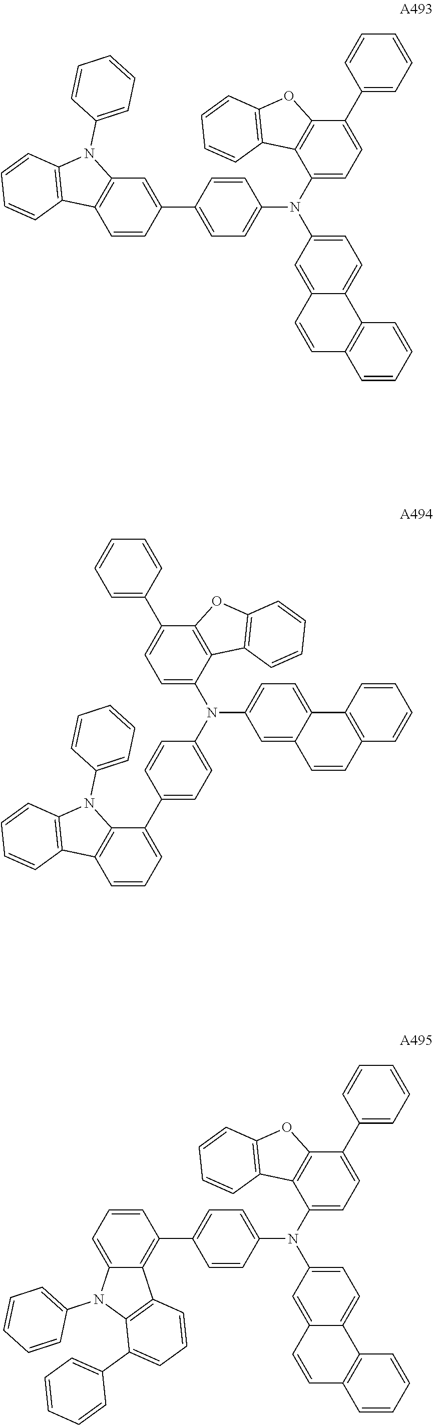 [SMILES: c1ccc(-c2ccc(N(c3ccc(-c4ccc5c6ccccc6n(-c6ccccc6)c5c4)cc3)c3ccc4c(ccc5ccccc54)c3)c3c2oc2ccccc23)cc1 ; c1ccc(-c2ccc(N(c3ccc(-c4cccc5c6ccccc6n(-c6ccccc6)c45)cc3)c3ccc4c(ccc5ccccc54)c3)c3c2oc2ccccc23)cc1 ; c1ccc(-c2ccc(N(c3ccc(-c4cccc5c4c4cccc(-c6ccccc6)c4n5-c4ccccc4)cc3)c3ccc4c(ccc5ccccc54)c3)c3c2oc2ccccc23)cc1] 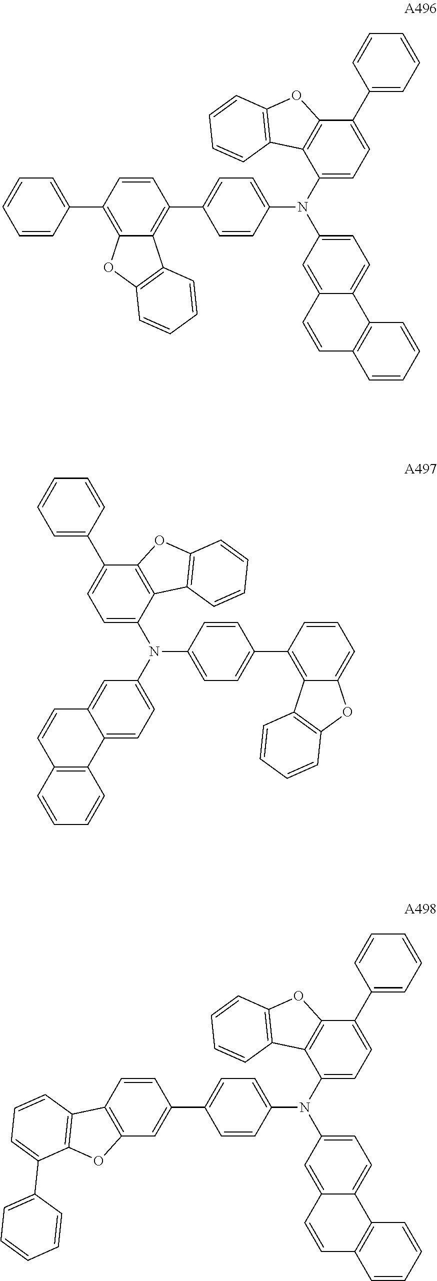 [SMILES: c1ccc(-c2ccc(-c3ccc(N(c4ccc5c(ccc6ccccc65)c4)c4ccc(-c5ccccc5)c5oc6ccccc6c45)cc3)c3c2oc2ccccc23)cc1 ; c1ccc(-c2ccc(N(c3ccc(-c4cccc5oc6ccccc6c45)cc3)c3ccc4c(ccc5ccccc54)c3)c3c2oc2ccccc23)cc1 ; c1ccc(-c2cccc3c2oc2cc(-c4ccc(N(c5ccc6c(ccc7ccccc76)c5)c5ccc(-c6ccccc6)c6oc7ccccc7c56)cc4)ccc23)cc1] 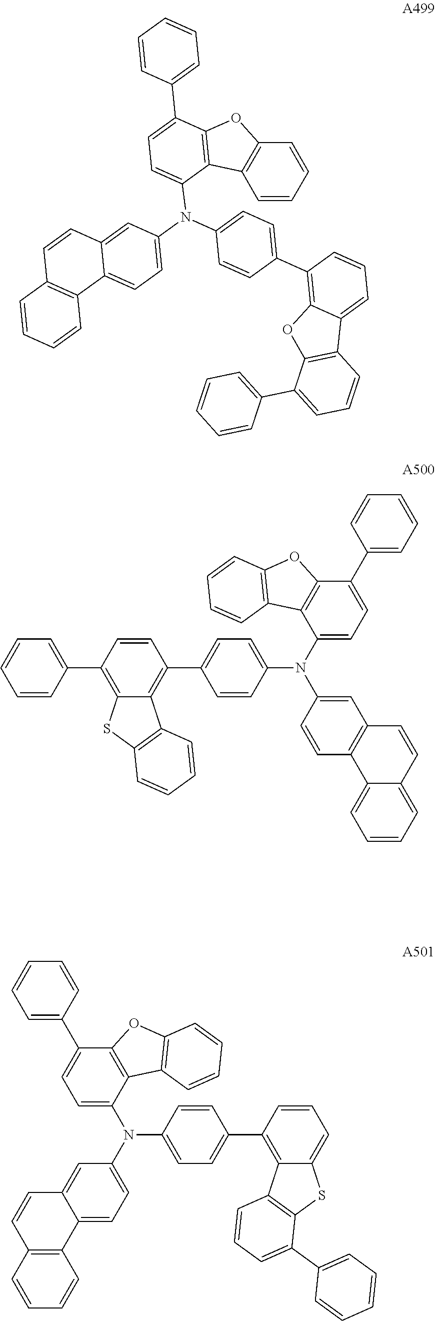 [SMILES: c1ccc(-c2cccc3c2oc2c(-c4ccc(N(c5ccc6c(ccc7ccccc76)c5)c5ccc(-c6ccccc6)c6oc7ccccc7c56)cc4)cccc23)cc1 ; c1ccc(-c2ccc(N(c3ccc(-c4ccc(-c5ccccc5)c5sc6ccccc6c45)cc3)c3ccc4c(ccc5ccccc54)c3)c3c2oc2ccccc23)cc1 ; c1ccc(-c2ccc(N(c3ccc(-c4cccc5sc6c(-c7ccccc7)cccc6c45)cc3)c3ccc4c(ccc5ccccc54)c3)c3c2oc2ccccc23)cc1] 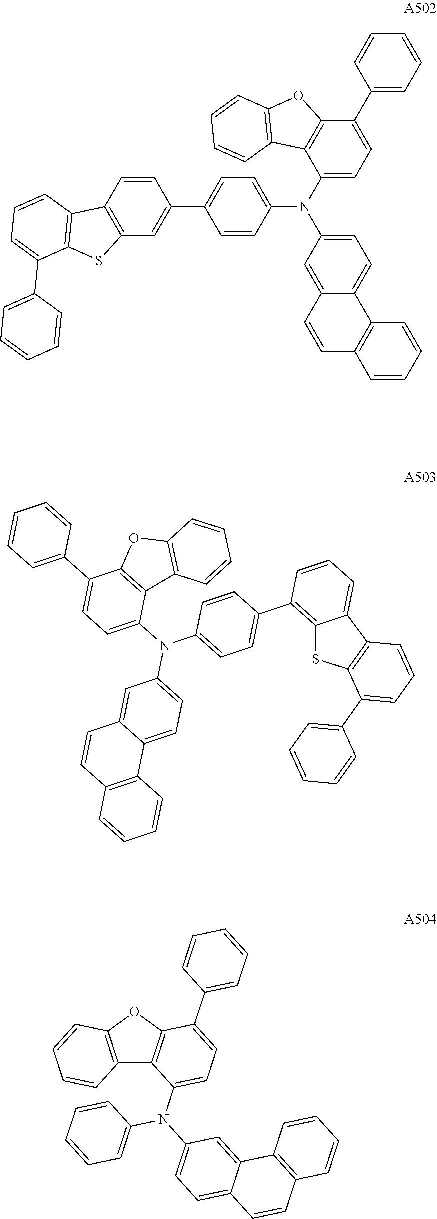 [SMILES: c1ccc(-c2ccc(N(c3ccc(-c4ccc5c(c4)sc4c(-c6ccccc6)cccc45)cc3)c3ccc4c(ccc5ccccc54)c3)c3c2oc2ccccc23)cc1 ; c1ccc(-c2ccc(N(c3ccc(-c4cccc5c4sc4c(-c6ccccc6)cccc45)cc3)c3ccc4c(ccc5ccccc54)c3)c3c2oc2ccccc23)cc1 ; c1ccc(-c2ccc(N(c3ccccc3)c3ccc4ccc5ccccc5c4c3)c3c2oc2ccccc23)cc1] 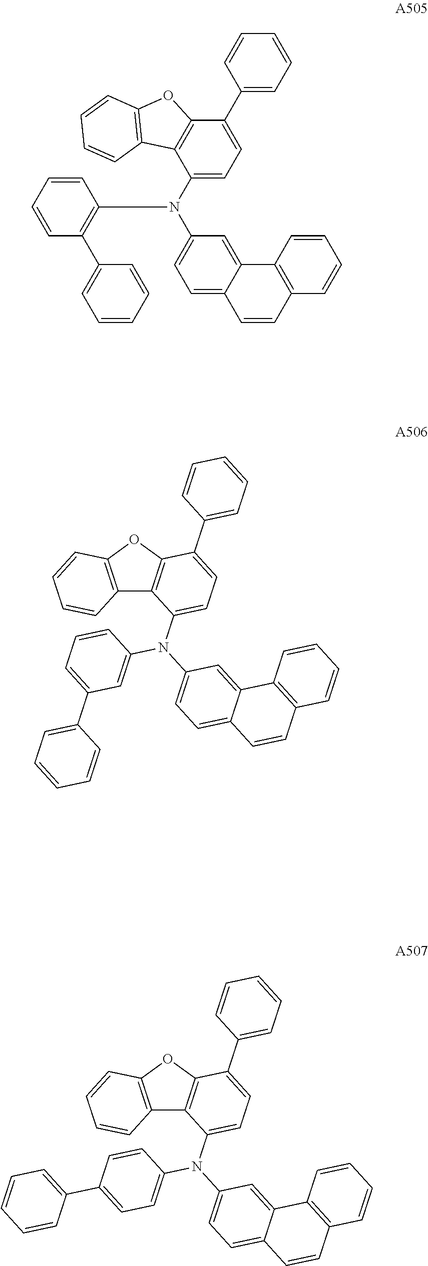 [SMILES: c1ccc(-c2ccccc2N(c2ccc3ccc4ccccc4c3c2)c2ccc(-c3ccccc3)c3oc4ccccc4c23)cc1 ; c1ccc(-c2cccc(N(c3ccc4ccc5ccccc5c4c3)c3ccc(-c4ccccc4)c4oc5ccccc5c34)c2)cc1 ; c1ccc(-c2ccc(N(c3ccc4ccc5ccccc5c4c3)c3ccc(-c4ccccc4)c4oc5ccccc5c34)cc2)cc1] 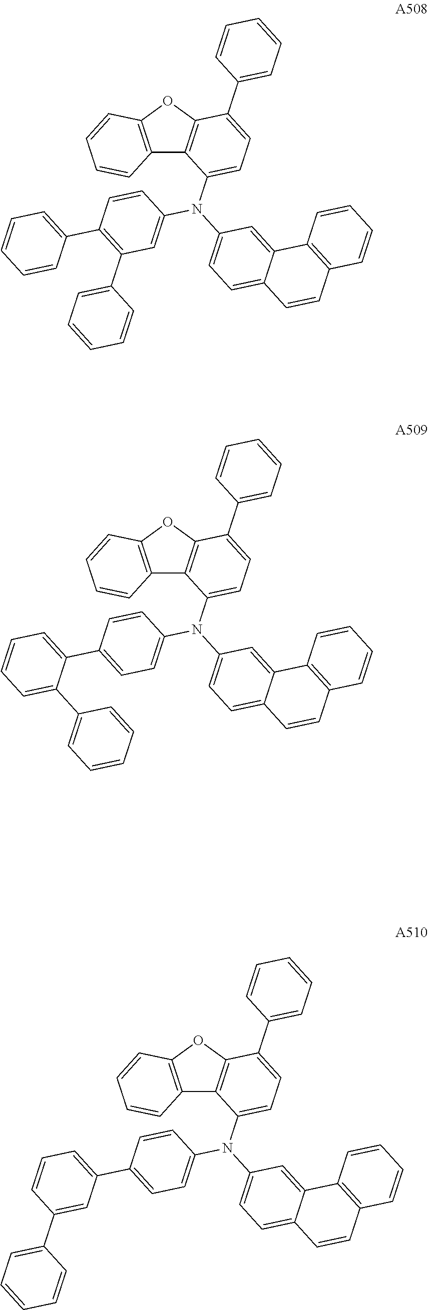 [SMILES: c1ccc(-c2ccc(N(c3ccc4ccc5ccccc5c4c3)c3ccc(-c4ccccc4)c4oc5ccccc5c34)cc2-c2ccccc2)cc1 ; c1ccc(-c2ccccc2-c2ccc(N(c3ccc4ccc5ccccc5c4c3)c3ccc(-c4ccccc4)c4oc5ccccc5c34)cc2)cc1 ; c1ccc(-c2cccc(-c3ccc(N(c4ccc5ccc6ccccc6c5c4)c4ccc(-c5ccccc5)c5oc6ccccc6c45)cc3)c2)cc1] 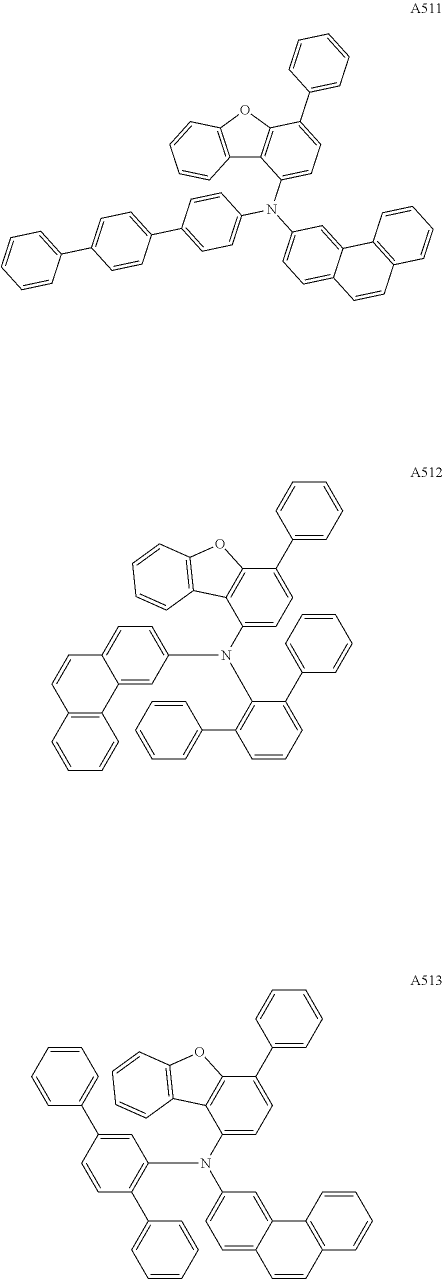 [SMILES: c1ccc(-c2ccc(-c3ccc(N(c4ccc5ccc6ccccc6c5c4)c4ccc(-c5ccccc5)c5oc6ccccc6c45)cc3)cc2)cc1 ; c1ccc(-c2cccc(-c3ccccc3)c2N(c2ccc3ccc4ccccc4c3c2)c2ccc(-c3ccccc3)c3oc4ccccc4c23)cc1 ; c1ccc(-c2ccc(-c3ccccc3)c(N(c3ccc4ccc5ccccc5c4c3)c3ccc(-c4ccccc4)c4oc5ccccc5c34)c2)cc1] 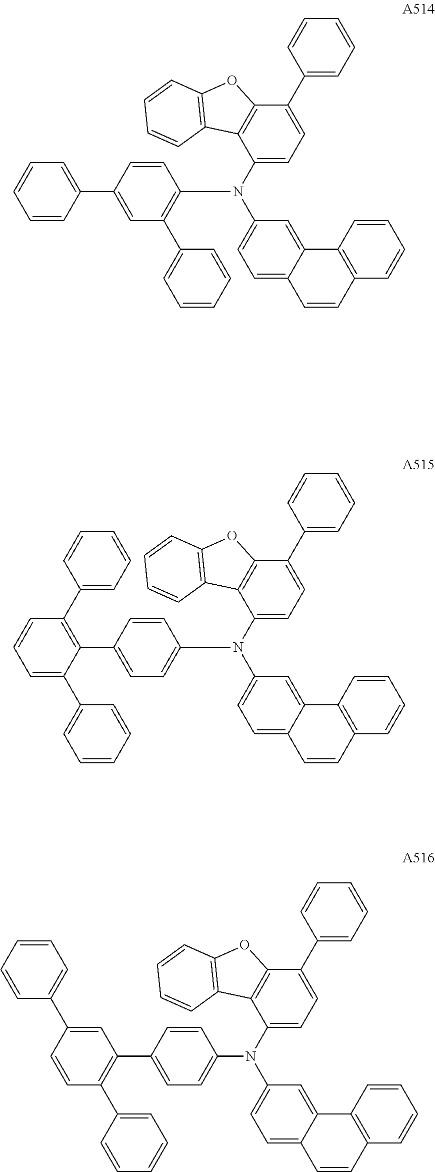 [SMILES: c1ccc(-c2ccc(N(c3ccc4ccc5ccccc5c4c3)c3ccc(-c4ccccc4)c4oc5ccccc5c34)c(-c3ccccc3)c2)cc1 ; c1ccc(-c2cccc(-c3ccccc3)c2-c2ccc(N(c3ccc4ccc5ccccc5c4c3)c3ccc(-c4ccccc4)c4oc5ccccc5c34)cc2)cc1 ; c1ccc(-c2ccc(-c3ccccc3)c(-c3ccc(N(c4ccc5ccc6ccccc6c5c4)c4ccc(-c5ccccc5)c5oc6ccccc6c45)cc3)c2)cc1] 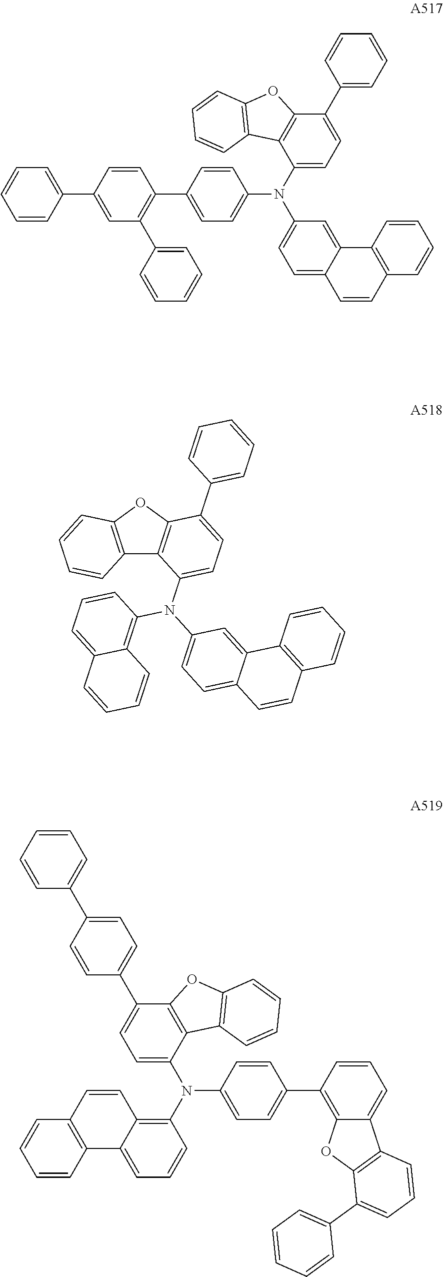 [SMILES: c1ccc(-c2ccc(-c3ccc(N(c4ccc5ccc6ccccc6c5c4)c4ccc(-c5ccccc5)c5oc6ccccc6c45)cc3)c(-c3ccccc3)c2)cc1 ; c1ccc(-c2ccc(N(c3ccc4ccc5ccccc5c4c3)c3cccc4ccccc34)c3c2oc2ccccc23)cc1 ; c1ccc(-c2ccc(-c3ccc(N(c4ccc(-c5cccc6c5oc5c(-c7ccccc7)cccc56)cc4)c4cccc5c4ccc4ccccc45)c4c3oc3ccccc34)cc2)cc1] 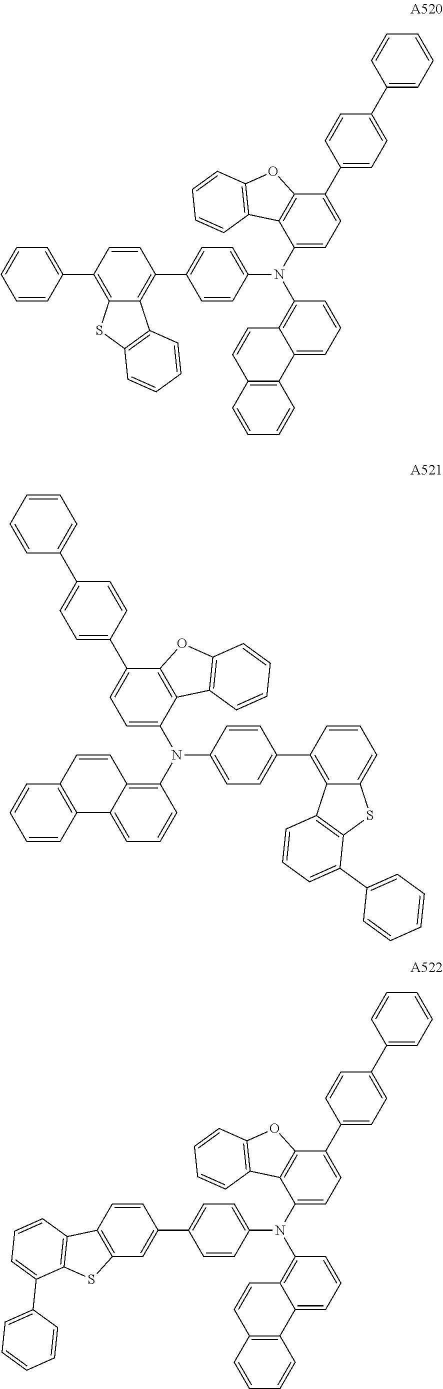 [SMILES: c1ccc(-c2ccc(-c3ccc(N(c4ccc(-c5ccc(-c6ccccc6)c6sc7ccccc7c56)cc4)c4cccc5c4ccc4ccccc45)c4c3oc3ccccc34)cc2)cc1 ; c1ccc(-c2ccc(-c3ccc(N(c4ccc(-c5cccc6sc7c(-c8ccccc8)cccc7c56)cc4)c4cccc5c4ccc4ccccc45)c4c3oc3ccccc34)cc2)cc1 ; c1ccc(-c2ccc(-c3ccc(N(c4ccc(-c5ccc6c(c5)sc5c(-c7ccccc7)cccc56)cc4)c4cccc5c4ccc4ccccc45)c4c3oc3ccccc34)cc2)cc1] 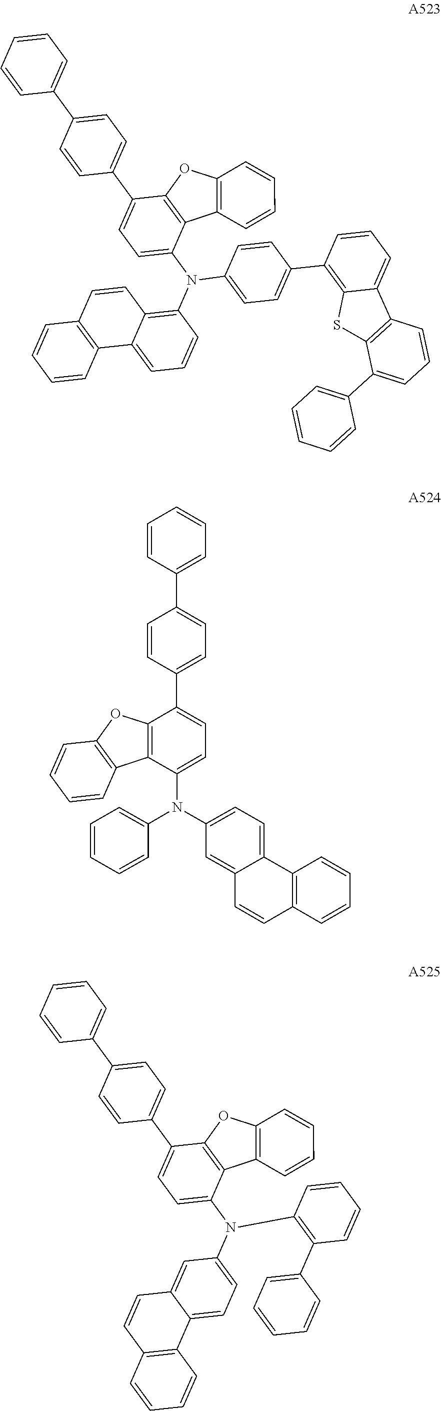 [SMILES: c1ccc(-c2ccc(-c3ccc(N(c4ccc(-c5cccc6c5sc5c(-c7ccccc7)cccc56)cc4)c4cccc5c4ccc4ccccc45)c4c3oc3ccccc34)cc2)cc1 ; c1ccc(-c2ccc(-c3ccc(N(c4ccccc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccccc4-c4ccccc4)c4c3oc3ccccc34)cc2)cc1] 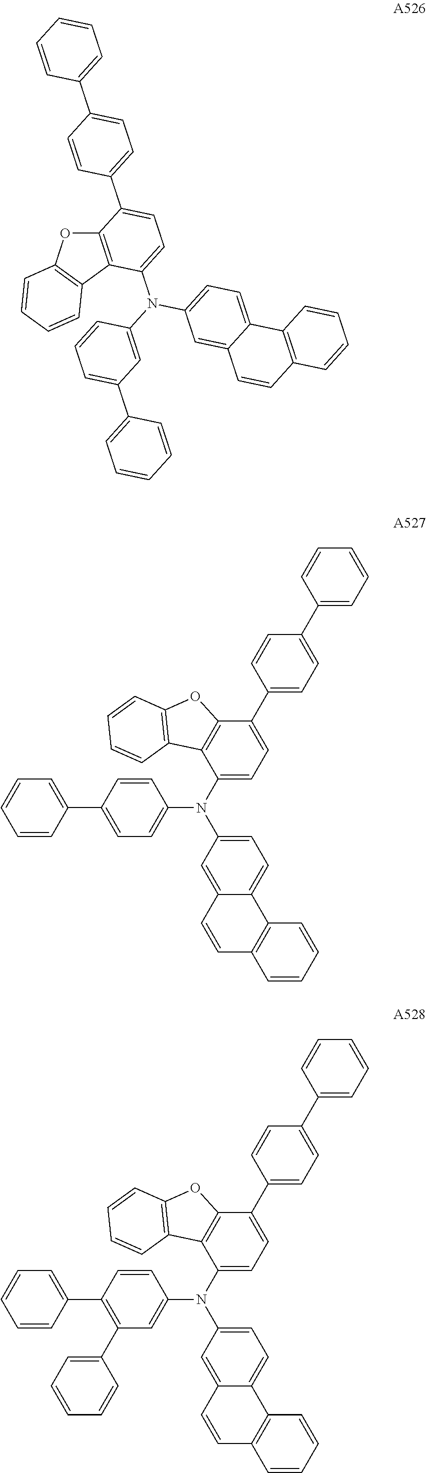 [SMILES: c1ccc(-c2ccc(-c3ccc(N(c4cccc(-c5ccccc5)c4)c4ccc5c(ccc6ccccc65)c4)c4c3oc3ccccc34)cc2)cc1 ; c1ccc(-c2ccc(-c3ccc(N(c4ccc(-c5ccccc5)cc4)c4ccc5c(ccc6ccccc65)c4)c4c3oc3ccccc34)cc2)cc1 ; c1ccc(-c2ccc(-c3ccc(N(c4ccc(-c5ccccc5)c(-c5ccccc5)c4)c4ccc5c(ccc6ccccc65)c4)c4c3oc3ccccc34)cc2)cc1] 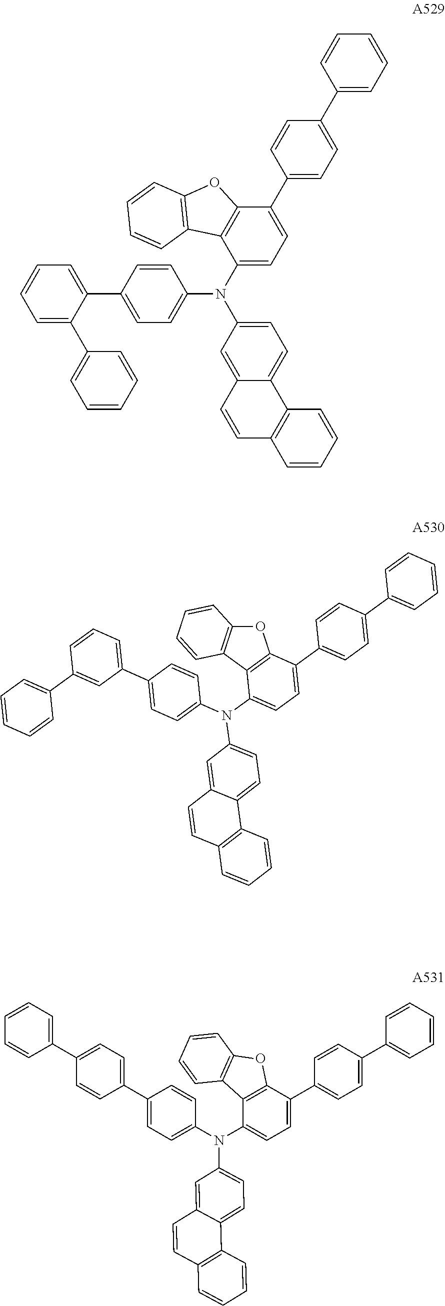 [SMILES: c1ccc(-c2ccc(-c3ccc(N(c4ccc(-c5ccccc5-c5ccccc5)cc4)c4ccc5c(ccc6ccccc65)c4)c4c3oc3ccccc34)cc2)cc1 ; c1ccc(-c2ccc(-c3ccc(N(c4ccc(-c5cccc(-c6ccccc6)c5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccc(-c6ccccc6)cc5)c5oc6ccccc6c45)cc3)cc2)cc1] 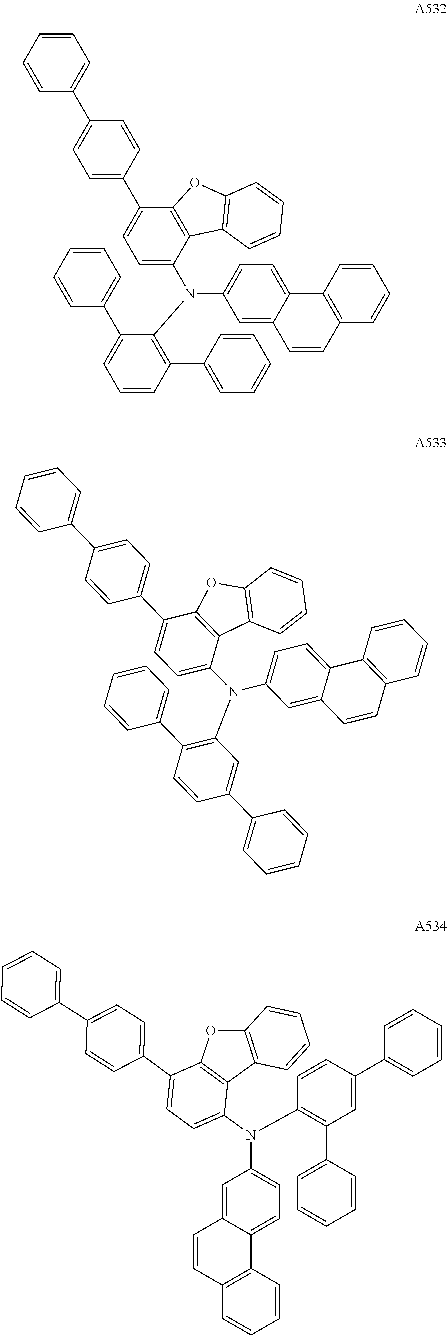 [SMILES: c1ccc(-c2ccc(-c3ccc(N(c4ccc5c(ccc6ccccc65)c4)c4c(-c5ccccc5)cccc4-c4ccccc4)c4c3oc3ccccc34)cc2)cc1 ; c1ccc(-c2ccc(-c3ccc(N(c4ccc5c(ccc6ccccc65)c4)c4cc(-c5ccccc5)ccc4-c4ccccc4)c4c3oc3ccccc34)cc2)cc1 ; c1ccc(-c2ccc(-c3ccc(N(c4ccc5c(ccc6ccccc65)c4)c4ccc(-c5ccccc5)cc4-c4ccccc4)c4c3oc3ccccc34)cc2)cc1] 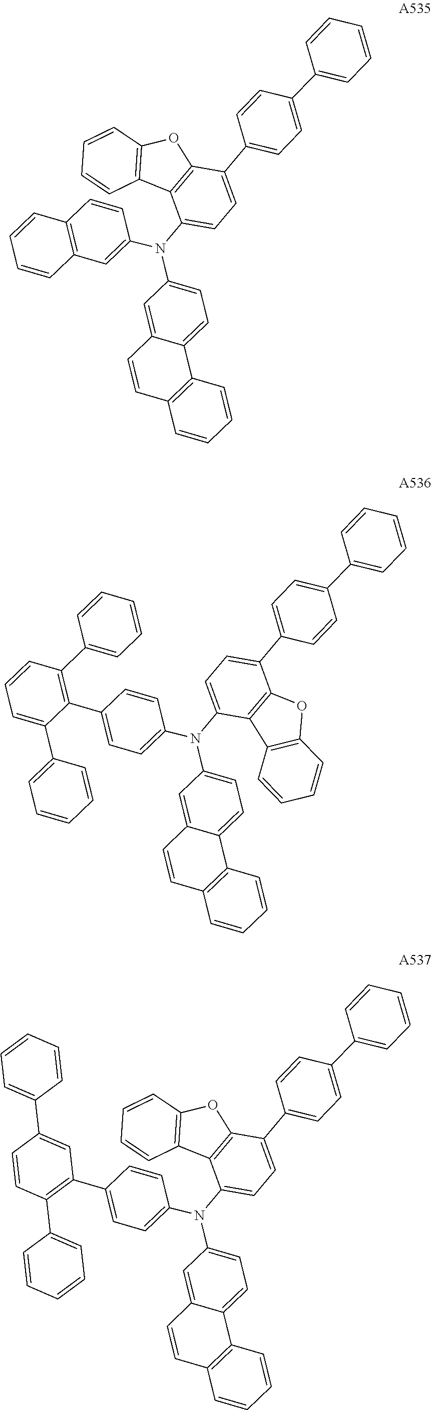 [SMILES: c1ccc(-c2ccc(-c3ccc(N(c4ccc5ccccc5c4)c4ccc5c(ccc6ccccc65)c4)c4c3oc3ccccc34)cc2)cc1 ; c1ccc(-c2ccc(-c3ccc(N(c4ccc(-c5c(-c6ccccc6)cccc5-c5ccccc5)cc4)c4ccc5c(ccc6ccccc65)c4)c4c3oc3ccccc34)cc2)cc1 ; c1ccc(-c2ccc(-c3ccc(N(c4ccc(-c5cc(-c6ccccc6)ccc5-c5ccccc5)cc4)c4ccc5c(ccc6ccccc65)c4)c4c3oc3ccccc34)cc2)cc1] 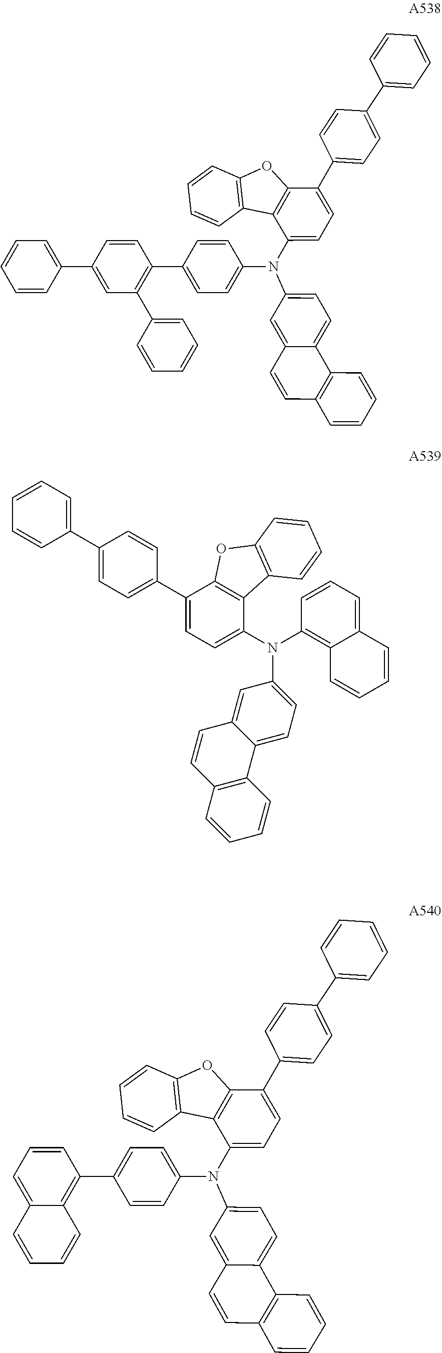 [SMILES: c1ccc(-c2ccc(-c3ccc(N(c4ccc(-c5ccc(-c6ccccc6)cc5-c5ccccc5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4cccc5ccccc45)c4c3oc3ccccc34)cc2)cc1 ; c1ccc(-c2ccc(-c3ccc(N(c4ccc(-c5cccc6ccccc56)cc4)c4ccc5c(ccc6ccccc65)c4)c4c3oc3ccccc34)cc2)cc1] 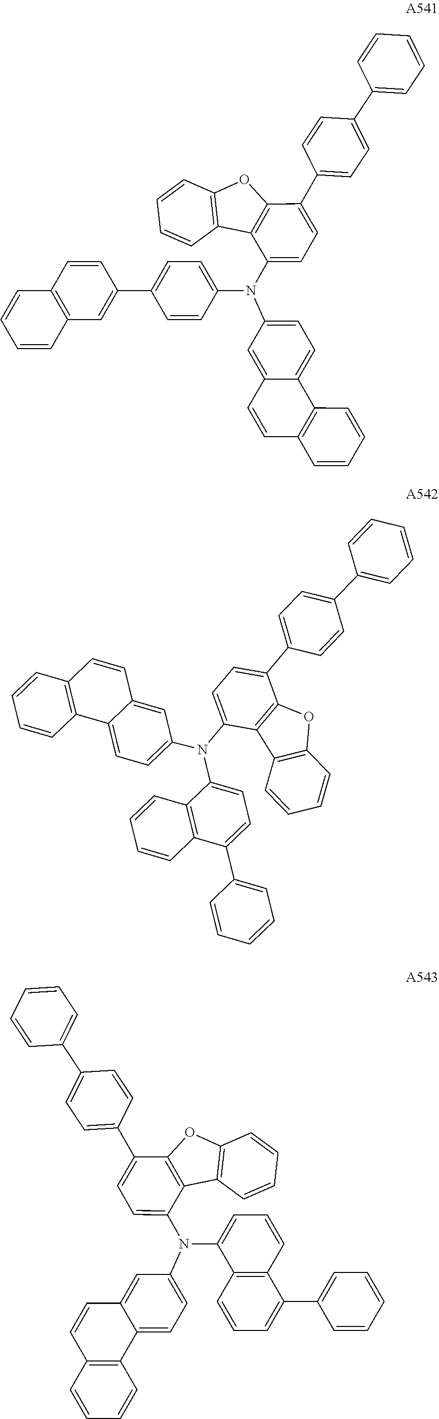 [SMILES: c1ccc(-c2ccc(-c3ccc(N(c4ccc(-c5ccc6ccccc6c5)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccccc5)c5ccccc45)c4c3oc3ccccc34)cc2)cc1 ; c1ccc(-c2ccc(-c3ccc(N(c4ccc5c(ccc6ccccc65)c4)c4cccc5c(-c6ccccc6)cccc45)c4c3oc3ccccc34)cc2)cc1] 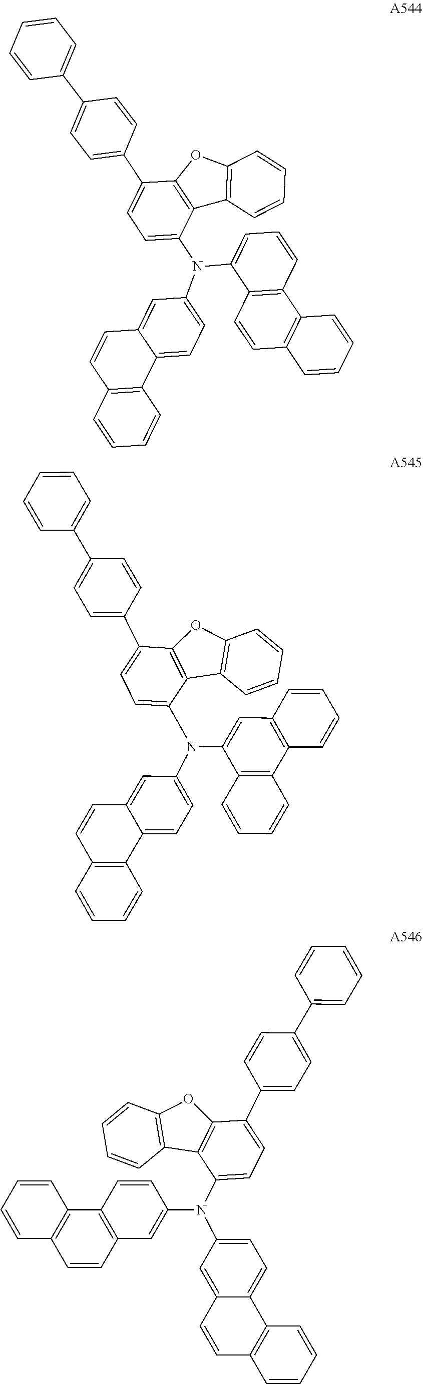 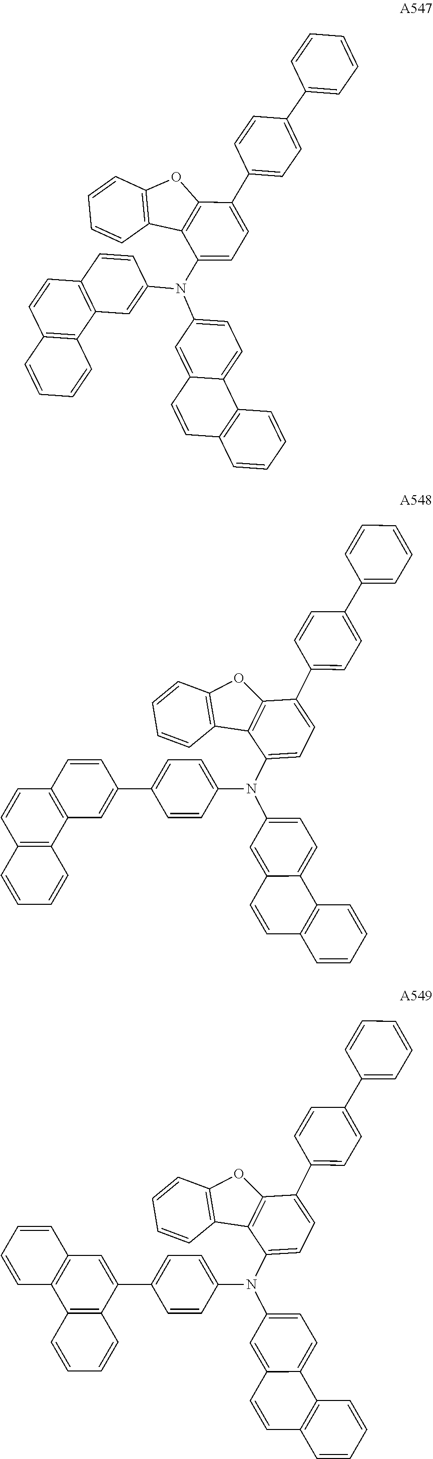 [SMILES: c1ccc(-c2ccc(-c3ccc(N(c4ccc5c(ccc6ccccc65)c4)c4ccc5ccc6ccccc6c5c4)c4c3oc3ccccc34)cc2)cc1 ; c1ccc(-c2ccc(-c3ccc(N(c4ccc(-c5ccc6ccc7ccccc7c6c5)cc4)c4ccc5c(ccc6ccccc65)c4)c4c3oc3ccccc34)cc2)cc1 ; c1ccc(-c2ccc(-c3ccc(N(c4ccc(-c5cc6ccccc6c6ccccc56)cc4)c4ccc5c(ccc6ccccc65)c4)c4c3oc3ccccc34)cc2)cc1] 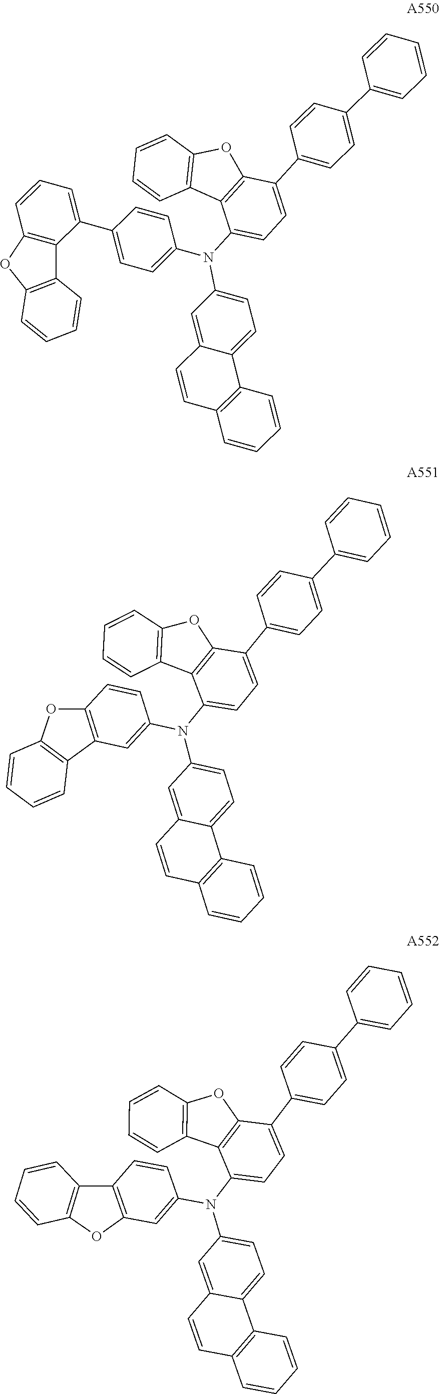 [SMILES: c1ccc(-c2ccc(-c3ccc(N(c4ccc(-c5cccc6oc7ccccc7c56)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc5oc6ccccc6c5c4)c4c3oc3ccccc34)cc2)cc1 ; c1ccc(-c2ccc(-c3ccc(N(c4ccc5c(ccc6ccccc65)c4)c4ccc5c(c4)oc4ccccc45)c4c3oc3ccccc34)cc2)cc1] 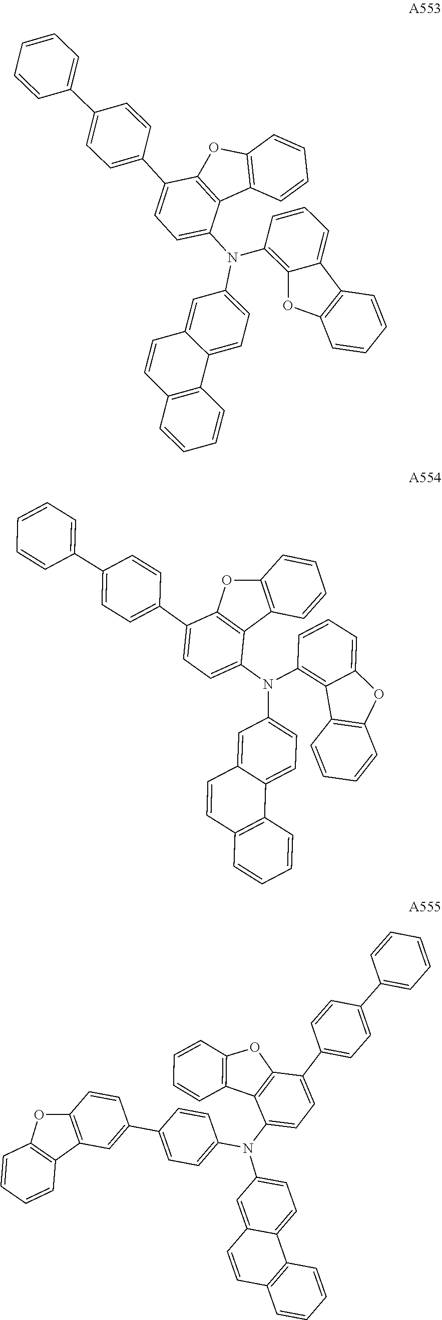 [SMILES: c1ccc(-c2ccc(-c3ccc(N(c4ccc5c(ccc6ccccc65)c4)c4cccc5c4oc4ccccc45)c4c3oc3ccccc34)cc2)cc1 ; c1ccc(-c2ccc(-c3ccc(N(c4ccc5c(ccc6ccccc65)c4)c4cccc5oc6ccccc6c45)c4c3oc3ccccc34)cc2)cc1 ; c1ccc(-c2ccc(-c3ccc(N(c4ccc(-c5ccc6oc7ccccc7c6c5)cc4)c4ccc5c(ccc6ccccc65)c4)c4c3oc3ccccc34)cc2)cc1] 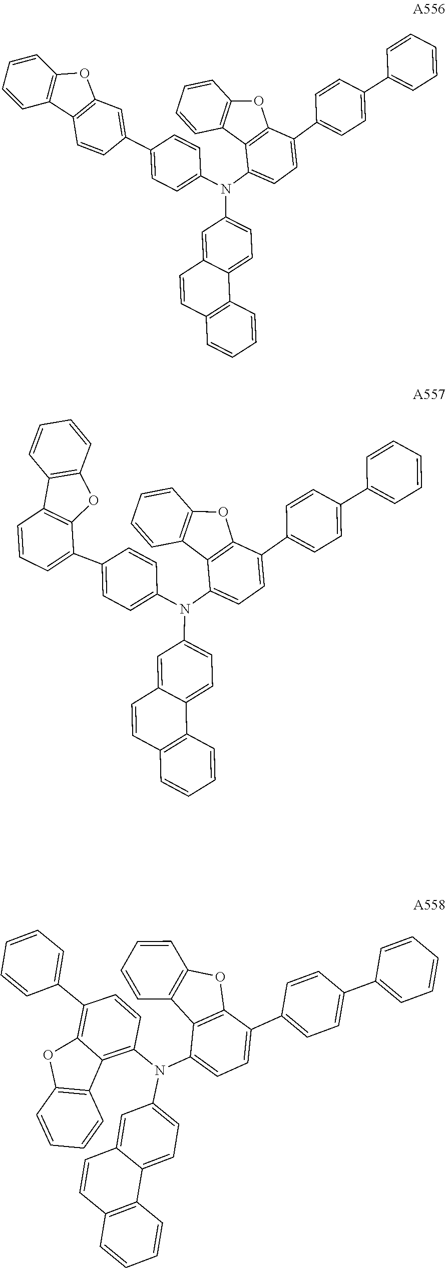 [SMILES: c1ccc(-c2ccc(-c3ccc(N(c4ccc(-c5ccc6c(c5)oc5ccccc56)cc4)c4ccc5c(ccc6ccccc65)c4)c4c3oc3ccccc34)cc2)cc1 ; c1ccc(-c2ccc(-c3ccc(N(c4ccc(-c5cccc6c5oc5ccccc56)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccccc5)c5oc6ccccc6c45)c4c3oc3ccccc34)cc2)cc1] 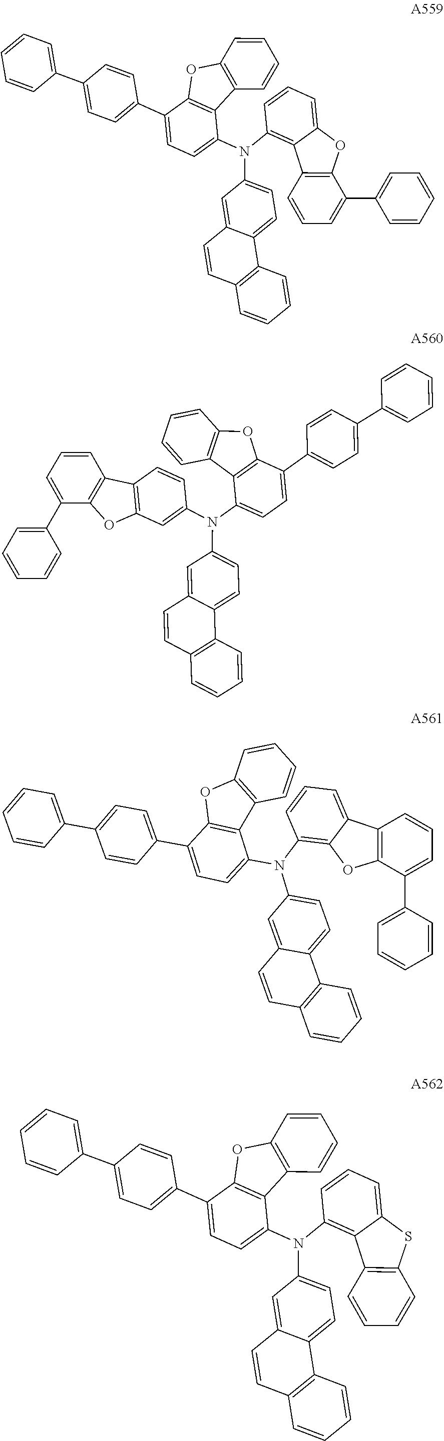 [SMILES: c1ccc(-c2ccc(-c3ccc(N(c4ccc5c(ccc6ccccc65)c4)c4cccc5oc6c(-c7ccccc7)cccc6c45)c4c3oc3ccccc34)cc2)cc1 ; c1ccc(-c2ccc(-c3ccc(N(c4ccc5c(ccc6ccccc65)c4)c4ccc5c(c4)oc4c(-c6ccccc6)cccc45)c4c3oc3ccccc34)cc2)cc1 ; c1ccc(-c2ccc(-c3ccc(N(c4ccc5c(ccc6ccccc65)c4)c4cccc5c4oc4c(-c6ccccc6)cccc45)c4c3oc3ccccc34)cc2)cc1 ; c1ccc(-c2ccc(-c3ccc(N(c4ccc5c(ccc6ccccc65)c4)c4cccc5sc6ccccc6c45)c4c3oc3ccccc34)cc2)cc1] 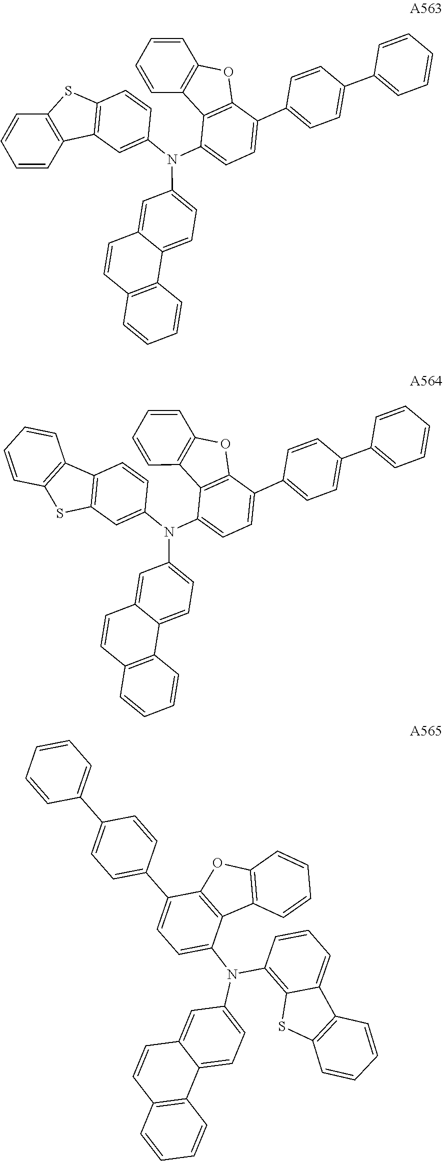 [SMILES: c1ccc(-c2ccc(-c3ccc(N(c4ccc5c(ccc6ccccc65)c4)c4ccc5sc6ccccc6c5c4)c4c3oc3ccccc34)cc2)cc1 ; c1ccc(-c2ccc(-c3ccc(N(c4ccc5c(ccc6ccccc65)c4)c4ccc5c(c4)sc4ccccc45)c4c3oc3ccccc34)cc2)cc1 ; c1ccc(-c2ccc(-c3ccc(N(c4ccc5c(ccc6ccccc65)c4)c4cccc5c4sc4ccccc45)c4c3oc3ccccc34)cc2)cc1] 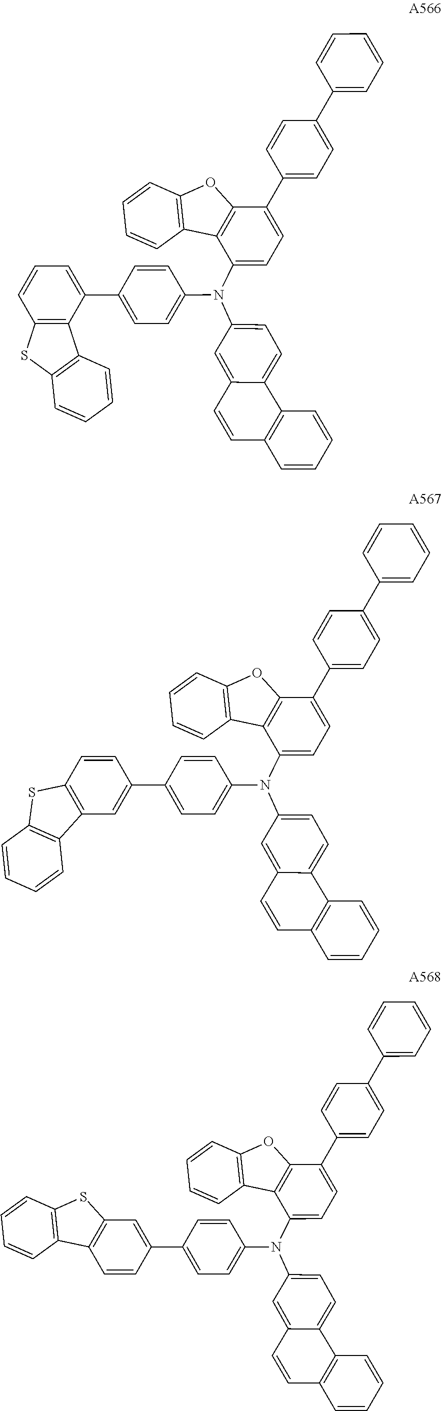 [SMILES: c1ccc(-c2ccc(-c3ccc(N(c4ccc(-c5cccc6sc7ccccc7c56)cc4)c4ccc5c(ccc6ccccc65)c4)c4c3oc3ccccc34)cc2)cc1 ; c1ccc(-c2ccc(-c3ccc(N(c4ccc(-c5ccc6sc7ccccc7c6c5)cc4)c4ccc5c(ccc6ccccc65)c4)c4c3oc3ccccc34)cc2)cc1 ; c1ccc(-c2ccc(-c3ccc(N(c4ccc(-c5ccc6c(c5)sc5ccccc56)cc4)c4ccc5c(ccc6ccccc65)c4)c4c3oc3ccccc34)cc2)cc1] 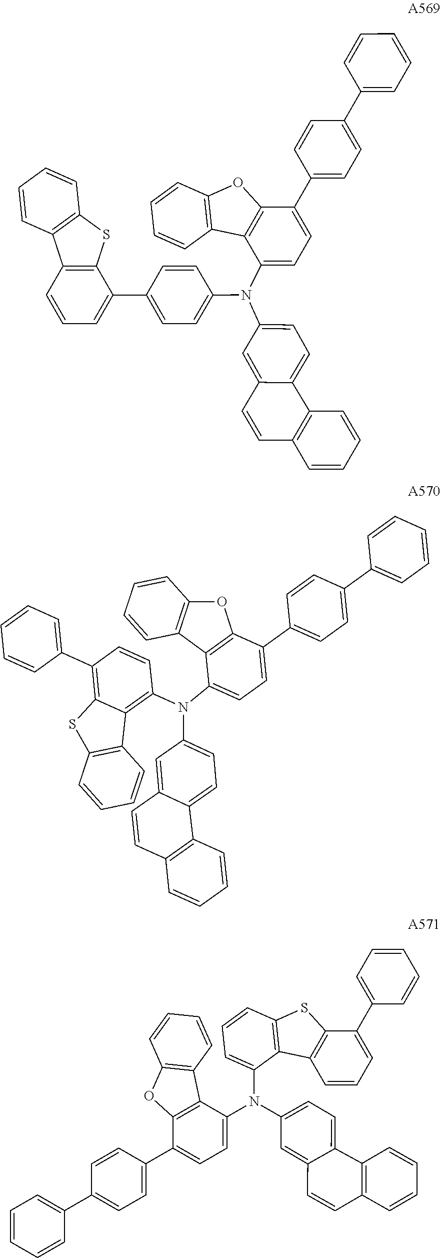 [SMILES: c1ccc(-c2ccc(-c3ccc(N(c4ccc(-c5cccc6c5sc5ccccc56)cc4)c4ccc5c(ccc6ccccc65)c4)c4c3oc3ccccc34)cc2)cc1 ; c1ccc(-c2ccc(-c3ccc(N(c4ccc5c(ccc6ccccc65)c4)c4ccc(-c5ccccc5)c5sc6ccccc6c45)c4c3oc3ccccc34)cc2)cc1 ; c1ccc(-c2ccc(-c3ccc(N(c4ccc5c(ccc6ccccc65)c4)c4cccc5sc6c(-c7ccccc7)cccc6c45)c4c3oc3ccccc34)cc2)cc1] 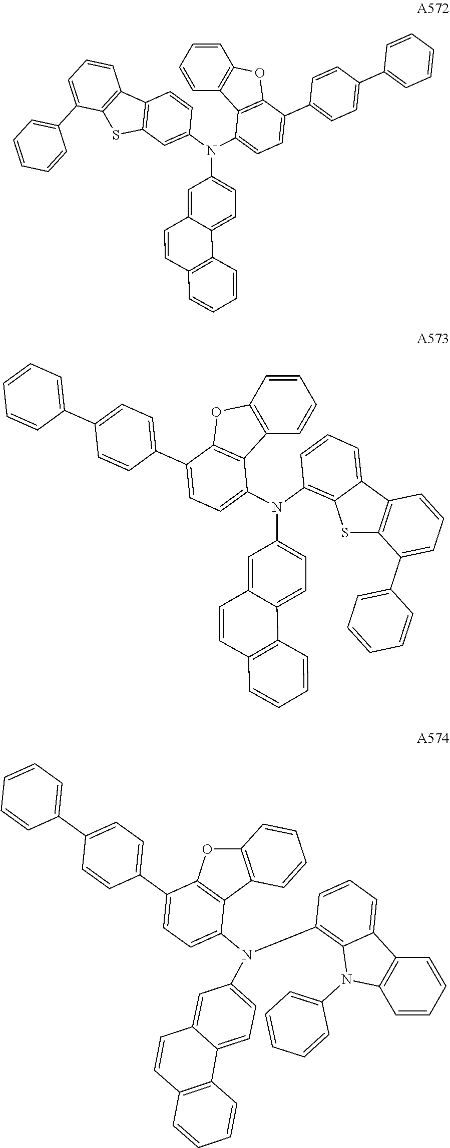 [SMILES: c1ccc(-c2ccc(-c3ccc(N(c4ccc5c(ccc6ccccc65)c4)c4ccc5c(c4)sc4c(-c6ccccc6)cccc45)c4c3oc3ccccc34)cc2)cc1 ; c1ccc(-c2ccc(-c3ccc(N(c4ccc5c(ccc6ccccc65)c4)c4cccc5c4sc4c(-c6ccccc6)cccc45)c4c3oc3ccccc34)cc2)cc1 ; c1ccc(-c2ccc(-c3ccc(N(c4ccc5c(ccc6ccccc65)c4)c4cccc5c6ccccc6n(-c6ccccc6)c45)c4c3oc3ccccc34)cc2)cc1] 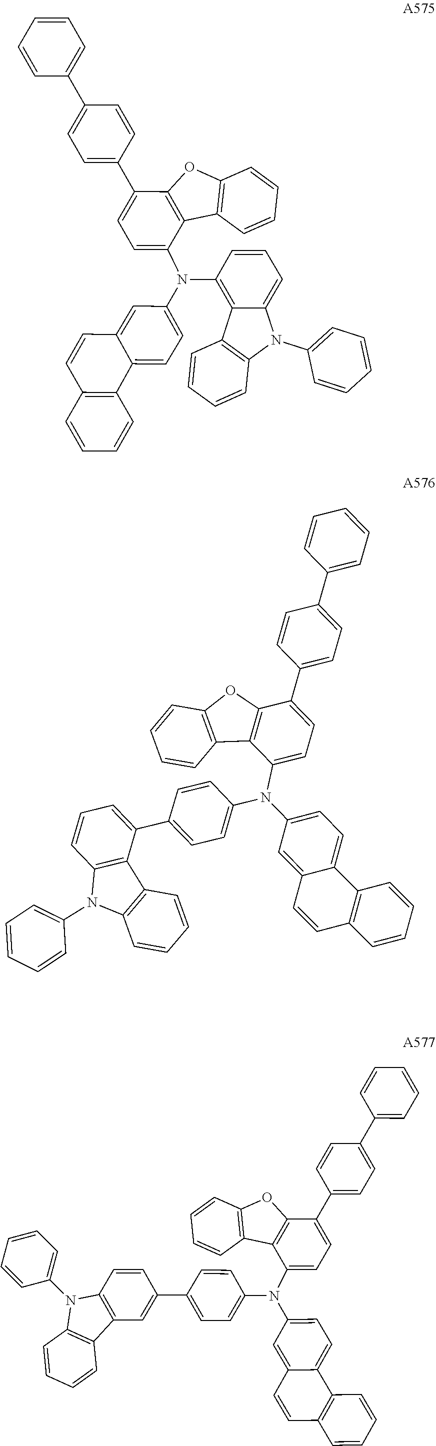 [SMILES: c1ccc(-c2ccc(-c3ccc(N(c4ccc5c(ccc6ccccc65)c4)c4cccc5c4c4ccccc4n5-c4ccccc4)c4c3oc3ccccc34)cc2)cc1 ; c1ccc(-c2ccc(-c3ccc(N(c4ccc(-c5cccc6c5c5ccccc5n6-c5ccccc5)cc4)c4ccc5c(ccc6ccccc65)c4)c4c3oc3ccccc34)cc2)cc1 ; c1ccc(-c2ccc(-c3ccc(N(c4ccc(-c5ccc6c(c5)c5ccccc5n6-c5ccccc5)cc4)c4ccc5c(ccc6ccccc65)c4)c4c3oc3ccccc34)cc2)cc1] 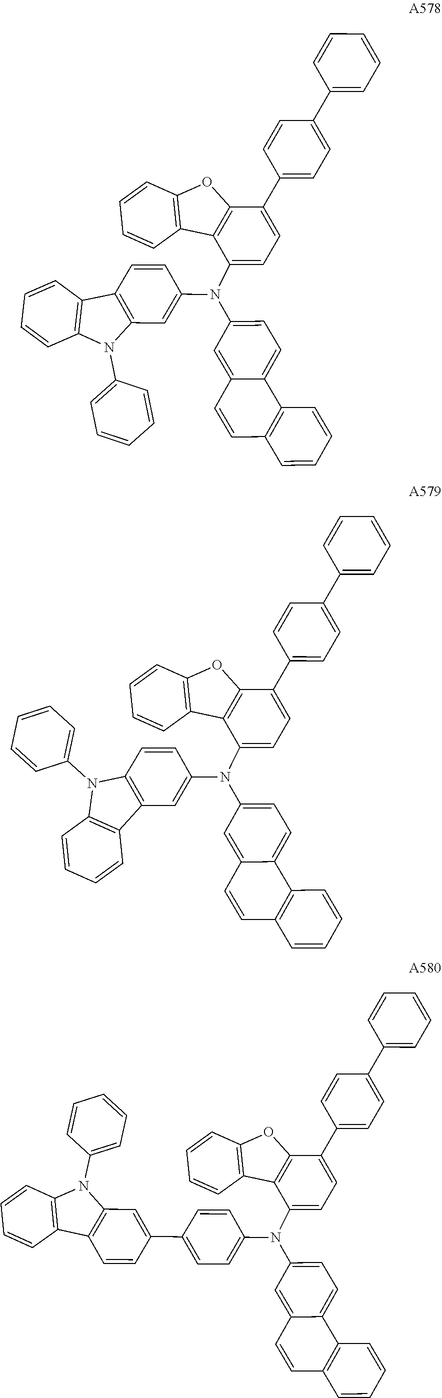 [SMILES: c1ccc(-c2ccc(-c3ccc(N(c4ccc5c(ccc6ccccc65)c4)c4ccc5c6ccccc6n(-c6ccccc6)c5c4)c4c3oc3ccccc34)cc2)cc1 ; c1ccc(-c2ccc(-c3ccc(N(c4ccc5c(ccc6ccccc65)c4)c4ccc5c(c4)c4ccccc4n5-c4ccccc4)c4c3oc3ccccc34)cc2)cc1 ; c1ccc(-c2ccc(-c3ccc(N(c4ccc(-c5ccc6c7ccccc7n(-c7ccccc7)c6c5)cc4)c4ccc5c(ccc6ccccc65)c4)c4c3oc3ccccc34)cc2)cc1] 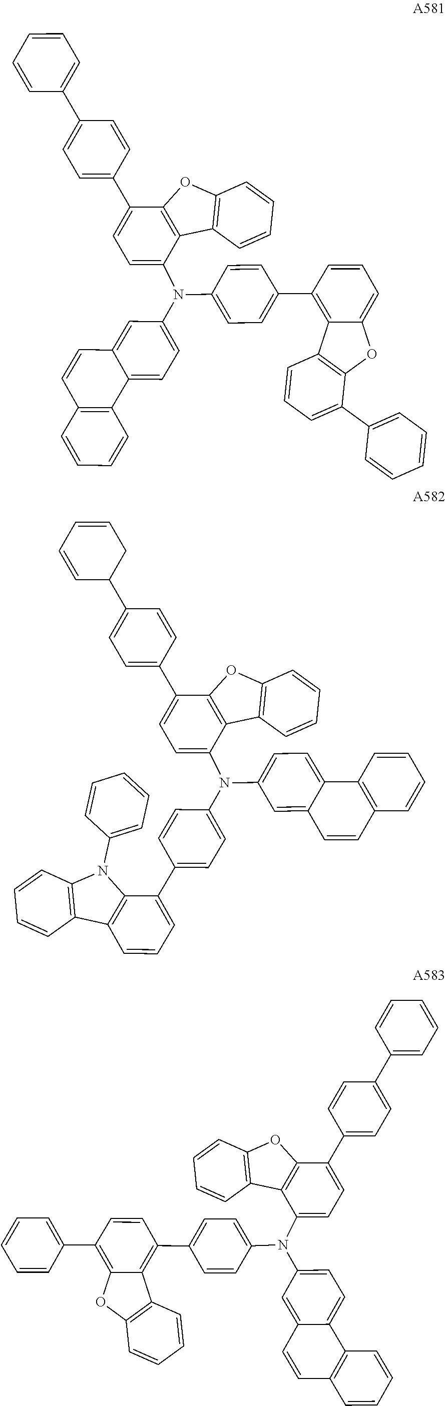 [SMILES: c1ccc(-c2ccc(-c3ccc(N(c4ccc(-c5cccc6oc7c(-c8ccccc8)cccc7c56)cc4)c4ccc5c(ccc6ccccc65)c4)c4c3oc3ccccc34)cc2)cc1 ; C1=CCC(c2ccc(-c3ccc(N(c4ccc(-c5cccc6c7ccccc7n(-c7ccccc7)c56)cc4)c4ccc5c(ccc6ccccc65)c4)c4c3oc3ccccc34)cc2)C=C1 ; c1ccc(-c2ccc(-c3ccc(N(c4ccc(-c5ccc(-c6ccccc6)c6oc7ccccc7c56)cc4)c4ccc5c(ccc6ccccc65)c4)c4c3oc3ccccc34)cc2)cc1] 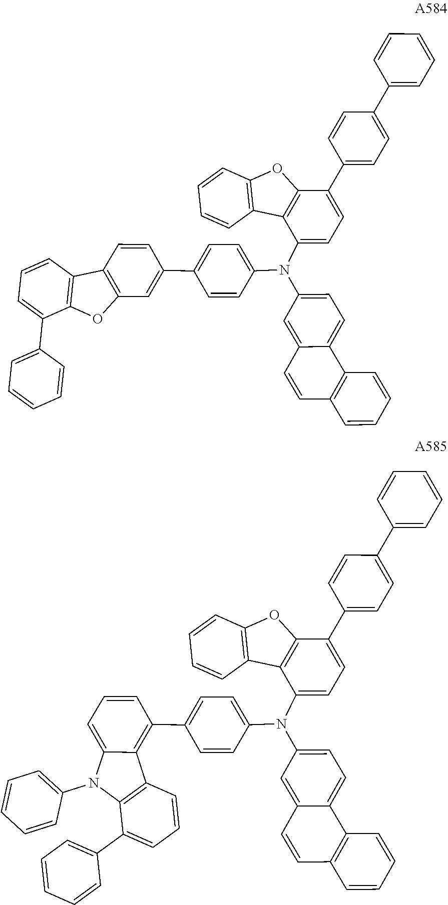 [SMILES: c1ccc(-c2ccc(-c3ccc(N(c4ccc(-c5ccc6c(c5)oc5c(-c7ccccc7)cccc56)cc4)c4ccc5c(ccc6ccccc65)c4)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4ccc5c(ccc6ccccc65)c4)c4c3oc3ccccc34)cc2)cc1] 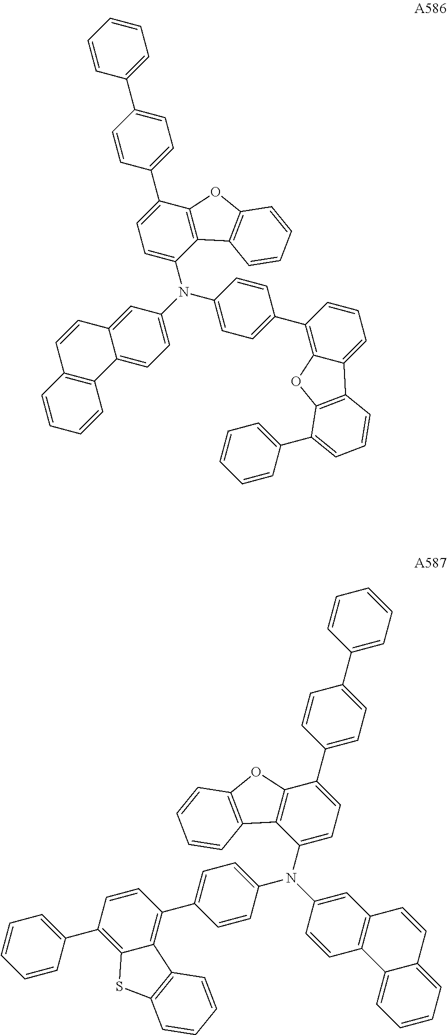 [SMILES: c1ccc(-c2ccc(-c3ccc(N(c4ccc(-c5cccc6c5oc5c(-c7ccccc7)cccc56)cc4)c4ccc5c(ccc6ccccc65)c4)c4c3oc3ccccc34)cc2)cc1 ; c1ccc(-c2ccc(-c3ccc(N(c4ccc(-c5ccc(-c6ccccc6)c6sc7ccccc7c56)cc4)c4ccc5c(ccc6ccccc65)c4)c4c3oc3ccccc34)cc2)cc1] 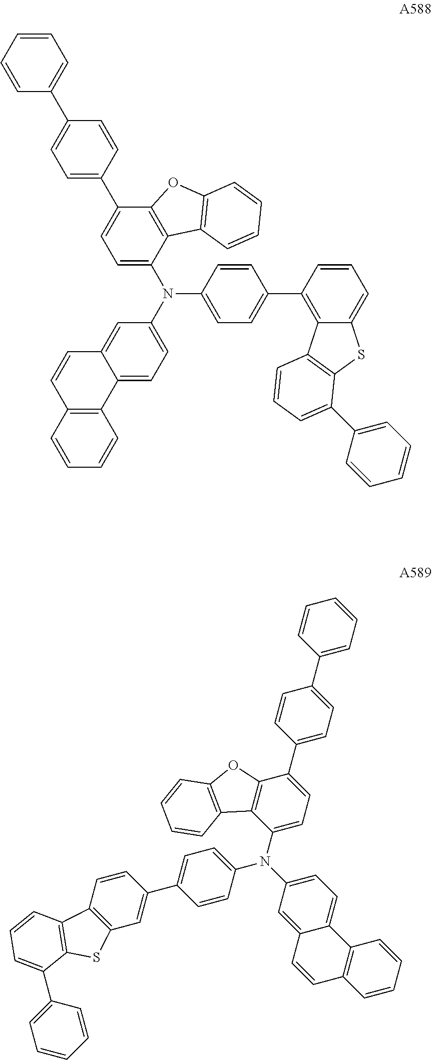 [SMILES: c1ccc(-c2ccc(-c3ccc(N(c4ccc(-c5cccc6sc7c(-c8ccccc8)cccc7c56)cc4)c4ccc5c(ccc6ccccc65)c4)c4c3oc3ccccc34)cc2)cc1 ; c1ccc(-c2ccc(-c3ccc(N(c4ccc(-c5ccc6c(c5)sc5c(-c7ccccc7)cccc56)cc4)c4ccc5c(ccc6ccccc65)c4)c4c3oc3ccccc34)cc2)cc1] 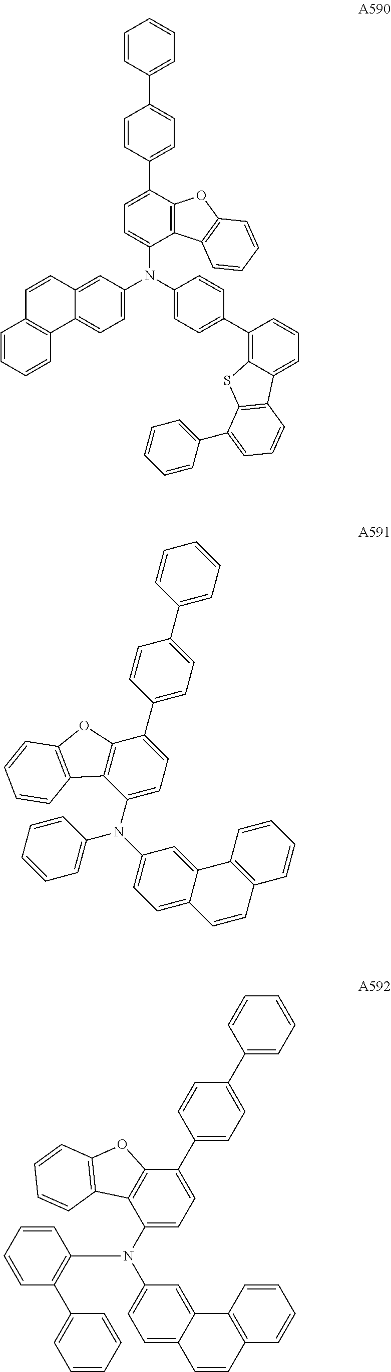 [SMILES: c1ccc(-c2ccc(-c3ccc(N(c4ccc(-c5cccc6c5sc5c(-c7ccccc7)cccc56)cc4)c4ccc5c(ccc6ccccc65)c4)c4c3oc3ccccc34)cc2)cc1 ; c1ccc(-c2ccc(-c3ccc(N(c4ccccc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccccc4-c4ccccc4)c4c3oc3ccccc34)cc2)cc1] 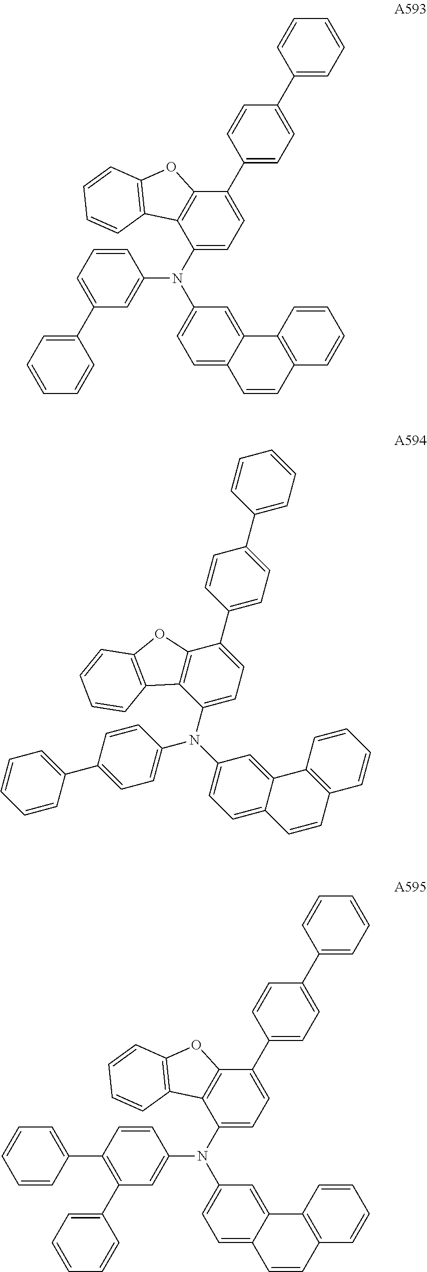 [SMILES: c1ccc(-c2ccc(-c3ccc(N(c4cccc(-c5ccccc5)c4)c4ccc5ccc6ccccc6c5c4)c4c3oc3ccccc34)cc2)cc1 ; c1ccc(-c2ccc(-c3ccc(N(c4ccc(-c5ccccc5)cc4)c4ccc5ccc6ccccc6c5c4)c4c3oc3ccccc34)cc2)cc1 ; c1ccc(-c2ccc(-c3ccc(N(c4ccc(-c5ccccc5)c(-c5ccccc5)c4)c4ccc5ccc6ccccc6c5c4)c4c3oc3ccccc34)cc2)cc1] 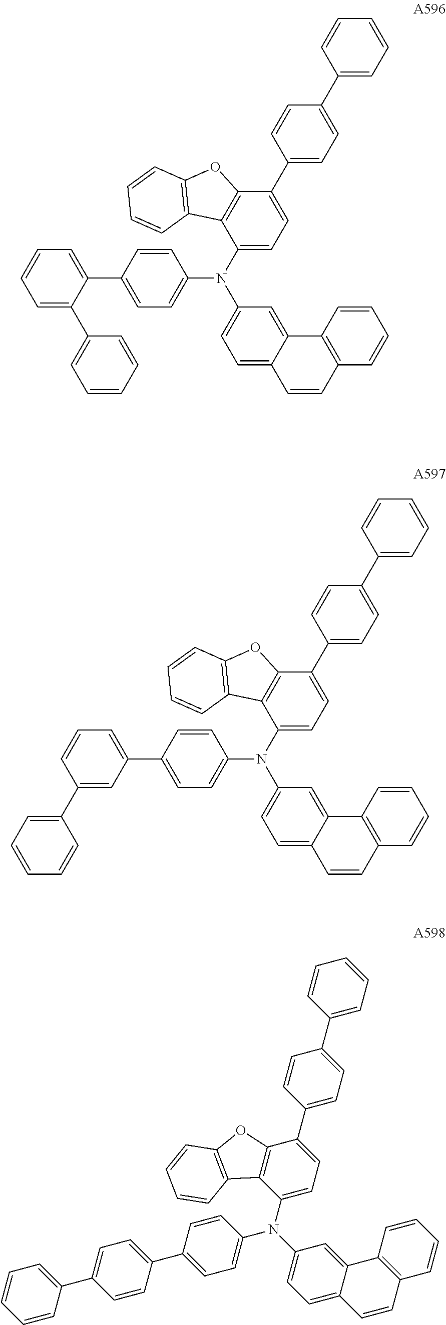 [SMILES: c1ccc(-c2ccc(-c3ccc(N(c4ccc(-c5ccccc5-c5ccccc5)cc4)c4ccc5ccc6ccccc6c5c4)c4c3oc3ccccc34)cc2)cc1 ; c1ccc(-c2ccc(-c3ccc(N(c4ccc(-c5cccc(-c6ccccc6)c5)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccc(-c6ccccc6)cc5)c5oc6ccccc6c45)cc3)cc2)cc1] 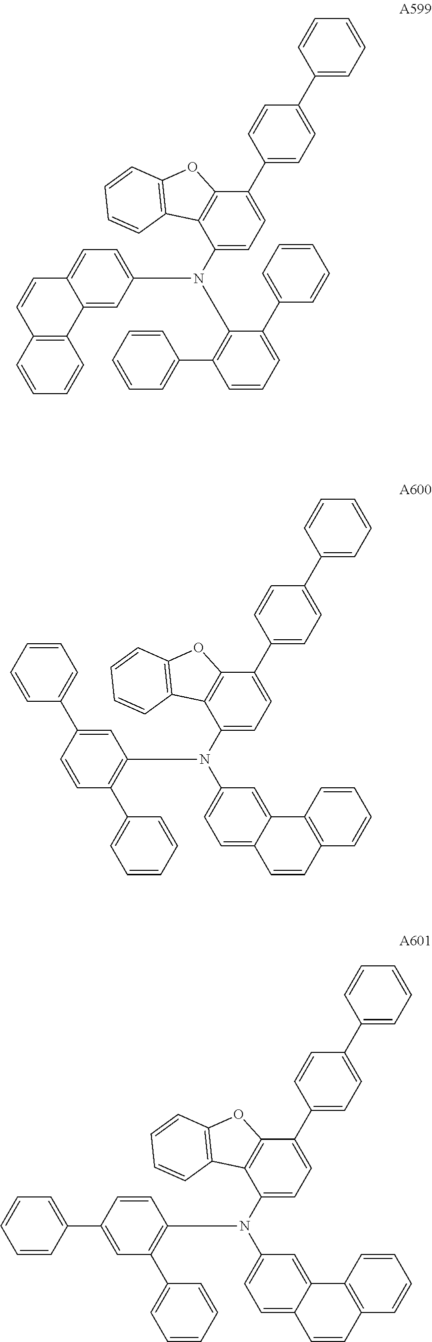 [SMILES: c1ccc(-c2ccc(-c3ccc(N(c4ccc5ccc6ccccc6c5c4)c4c(-c5ccccc5)cccc4-c4ccccc4)c4c3oc3ccccc34)cc2)cc1 ; c1ccc(-c2ccc(-c3ccc(N(c4ccc5ccc6ccccc6c5c4)c4cc(-c5ccccc5)ccc4-c4ccccc4)c4c3oc3ccccc34)cc2)cc1 ; c1ccc(-c2ccc(-c3ccc(N(c4ccc5ccc6ccccc6c5c4)c4ccc(-c5ccccc5)cc4-c4ccccc4)c4c3oc3ccccc34)cc2)cc1] 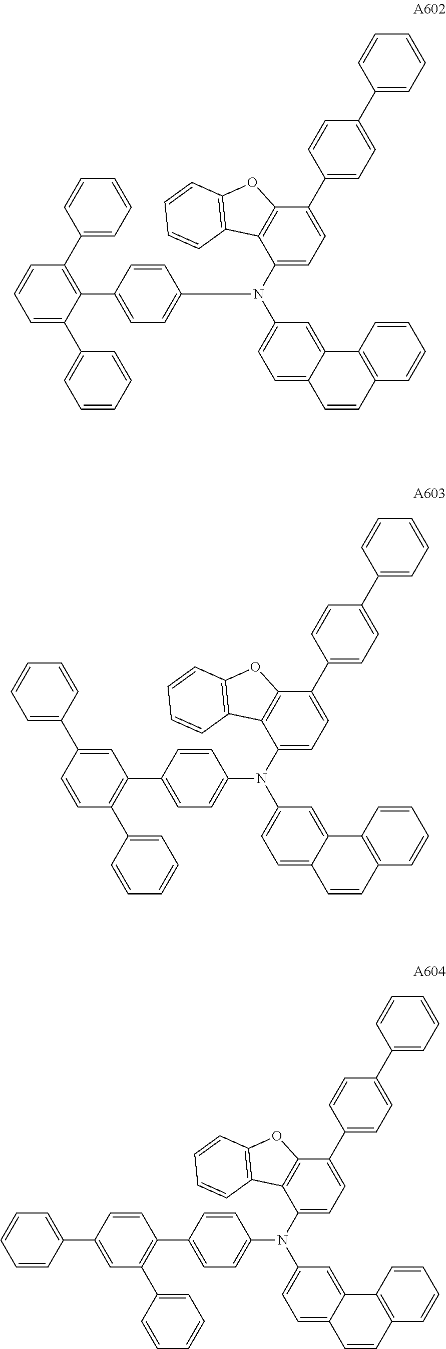 [SMILES: c1ccc(-c2ccc(-c3ccc(N(c4ccc(-c5c(-c6ccccc6)cccc5-c5ccccc5)cc4)c4ccc5ccc6ccccc6c5c4)c4c3oc3ccccc34)cc2)cc1 ; c1ccc(-c2ccc(-c3ccc(N(c4ccc(-c5cc(-c6ccccc6)ccc5-c5ccccc5)cc4)c4ccc5ccc6ccccc6c5c4)c4c3oc3ccccc34)cc2)cc1 ; c1ccc(-c2ccc(-c3ccc(N(c4ccc(-c5ccc(-c6ccccc6)cc5-c5ccccc5)cc4)c4ccc5ccc6ccccc6c5c4)c4c3oc3ccccc34)cc2)cc1] 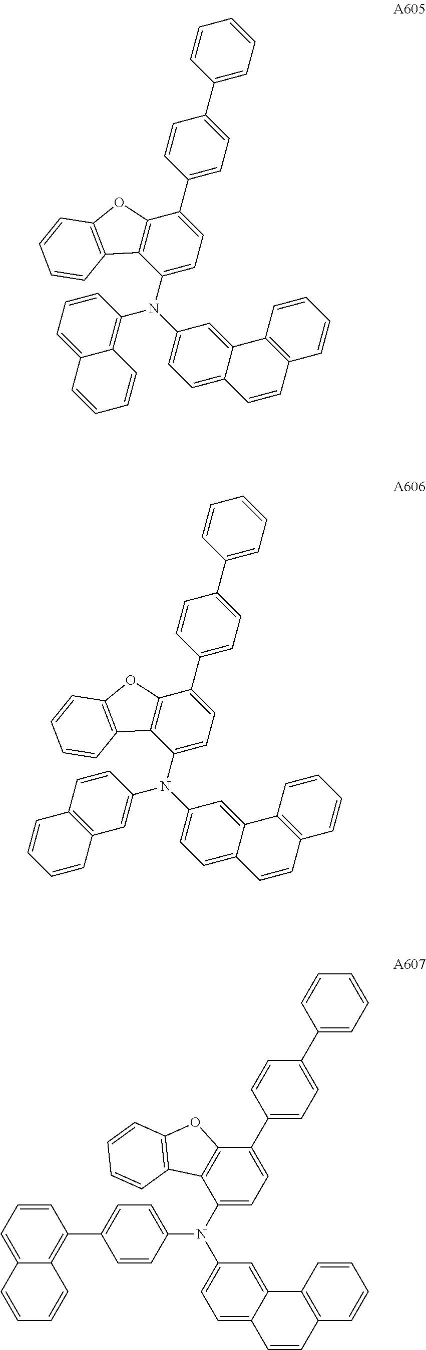 [SMILES: c1ccc(-c2ccc(-c3ccc(N(c4ccc5ccc6ccccc6c5c4)c4cccc5ccccc45)c4c3oc3ccccc34)cc2)cc1 ; c1ccc(-c2ccc(-c3ccc(N(c4ccc5ccccc5c4)c4ccc5ccc6ccccc6c5c4)c4c3oc3ccccc34)cc2)cc1 ; c1ccc(-c2ccc(-c3ccc(N(c4ccc(-c5cccc6ccccc56)cc4)c4ccc5ccc6ccccc6c5c4)c4c3oc3ccccc34)cc2)cc1] 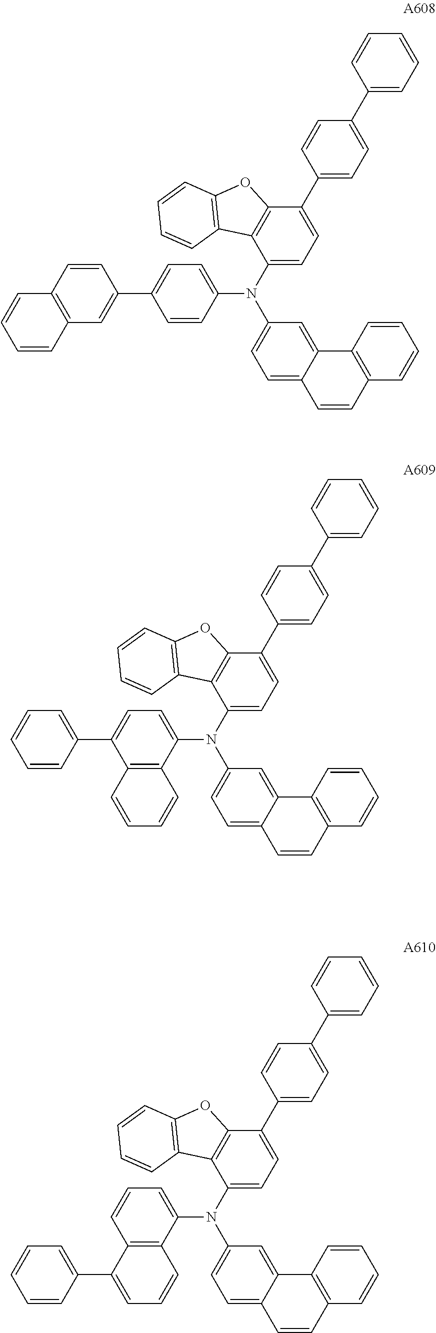 [SMILES: c1ccc(-c2ccc(-c3ccc(N(c4ccc(-c5ccc6ccccc6c5)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccccc5)c5ccccc45)c4c3oc3ccccc34)cc2)cc1 ; c1ccc(-c2ccc(-c3ccc(N(c4ccc5ccc6ccccc6c5c4)c4cccc5c(-c6ccccc6)cccc45)c4c3oc3ccccc34)cc2)cc1] 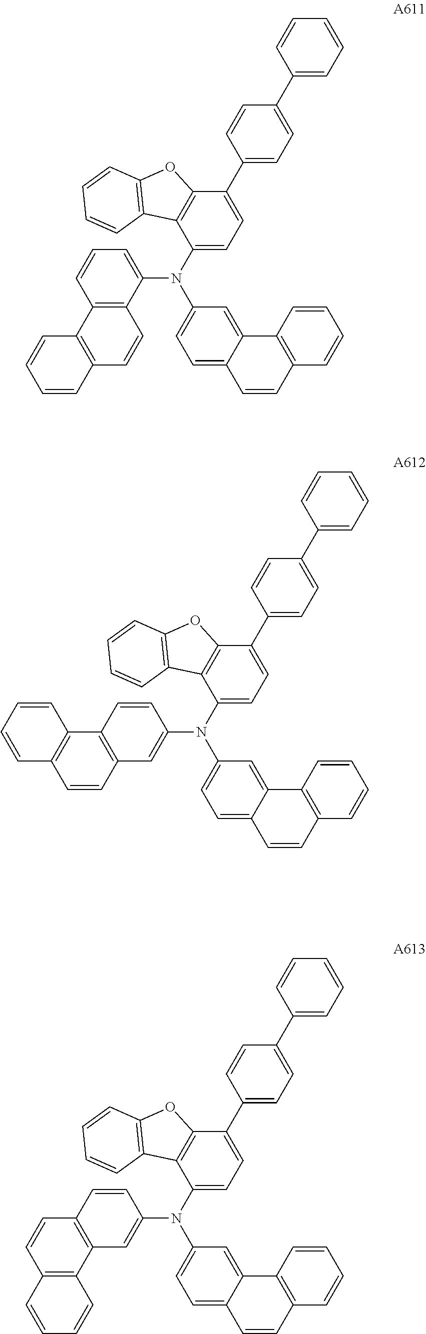 [SMILES: c1ccc(-c2ccc(-c3ccc(N(c4ccc5ccc6ccccc6c5c4)c4cccc5c4ccc4ccccc45)c4c3oc3ccccc34)cc2)cc1 ; c1ccc(-c2ccc(-c3ccc(N(c4ccc5c(ccc6ccccc65)c4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc5ccc6ccccc6c5c4)c4c3oc3ccccc34)cc2)cc1] 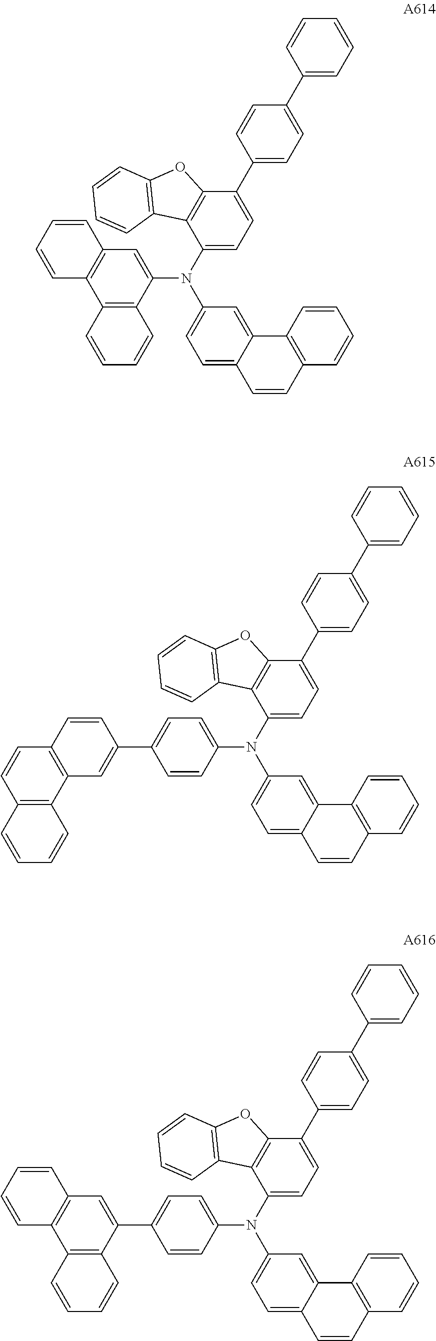 [SMILES: c1ccc(-c2ccc(-c3ccc(N(c4ccc5ccc6ccccc6c5c4)c4cc5ccccc5c5ccccc45)c4c3oc3ccccc34)cc2)cc1 ; c1ccc(-c2ccc(-c3ccc(N(c4ccc(-c5ccc6ccc7ccccc7c6c5)cc4)c4ccc5ccc6ccccc6c5c4)c4c3oc3ccccc34)cc2)cc1 ; c1ccc(-c2ccc(-c3ccc(N(c4ccc(-c5cc6ccccc6c6ccccc56)cc4)c4ccc5ccc6ccccc6c5c4)c4c3oc3ccccc34)cc2)cc1] 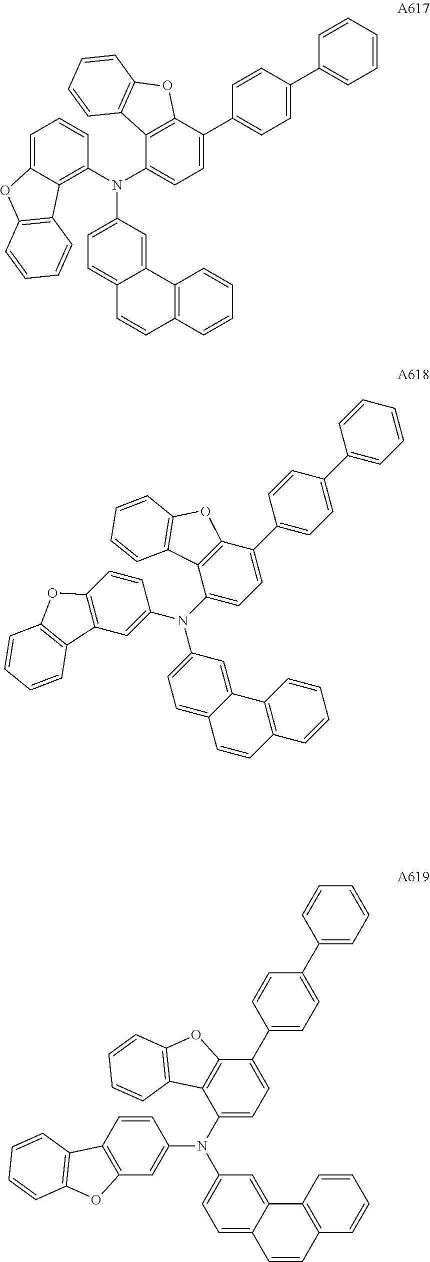 [SMILES: c1ccc(-c2ccc(-c3ccc(N(c4ccc5ccc6ccccc6c5c4)c4cccc5oc6ccccc6c45)c4c3oc3ccccc34)cc2)cc1 ; c1ccc(-c2ccc(-c3ccc(N(c4ccc5ccc6ccccc6c5c4)c4ccc5oc6ccccc6c5c4)c4c3oc3ccccc34)cc2)cc1 ; c1ccc(-c2ccc(-c3ccc(N(c4ccc5c(c4)oc4ccccc45)c4ccc5ccc6ccccc6c5c4)c4c3oc3ccccc34)cc2)cc1] 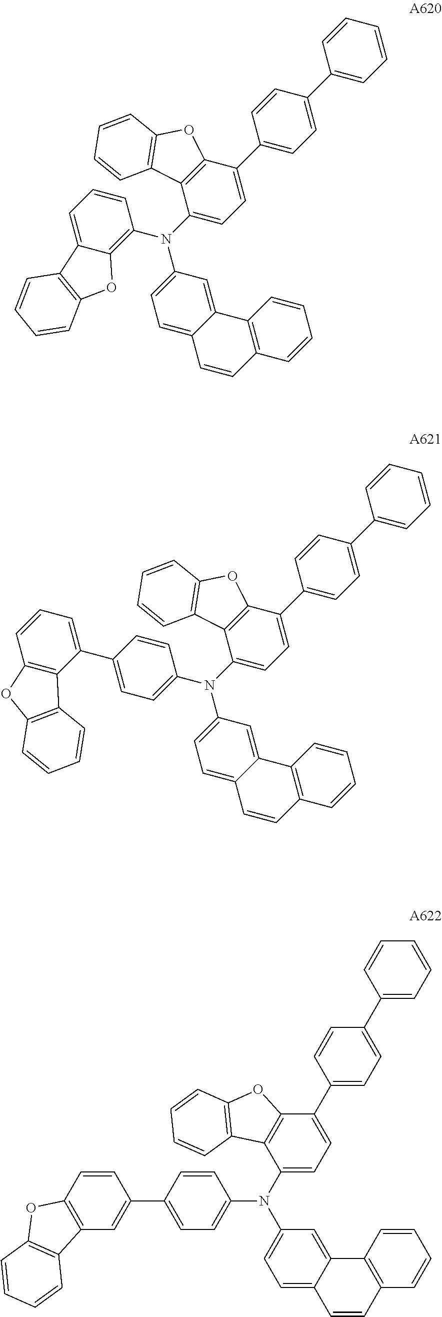 [SMILES: c1ccc(-c2ccc(-c3ccc(N(c4ccc5ccc6ccccc6c5c4)c4cccc5c4oc4ccccc45)c4c3oc3ccccc34)cc2)cc1 ; c1ccc(-c2ccc(-c3ccc(N(c4ccc(-c5cccc6oc7ccccc7c56)cc4)c4ccc5ccc6ccccc6c5c4)c4c3oc3ccccc34)cc2)cc1 ; c1ccc(-c2ccc(-c3ccc(N(c4ccc(-c5ccc6oc7ccccc7c6c5)cc4)c4ccc5ccc6ccccc6c5c4)c4c3oc3ccccc34)cc2)cc1] 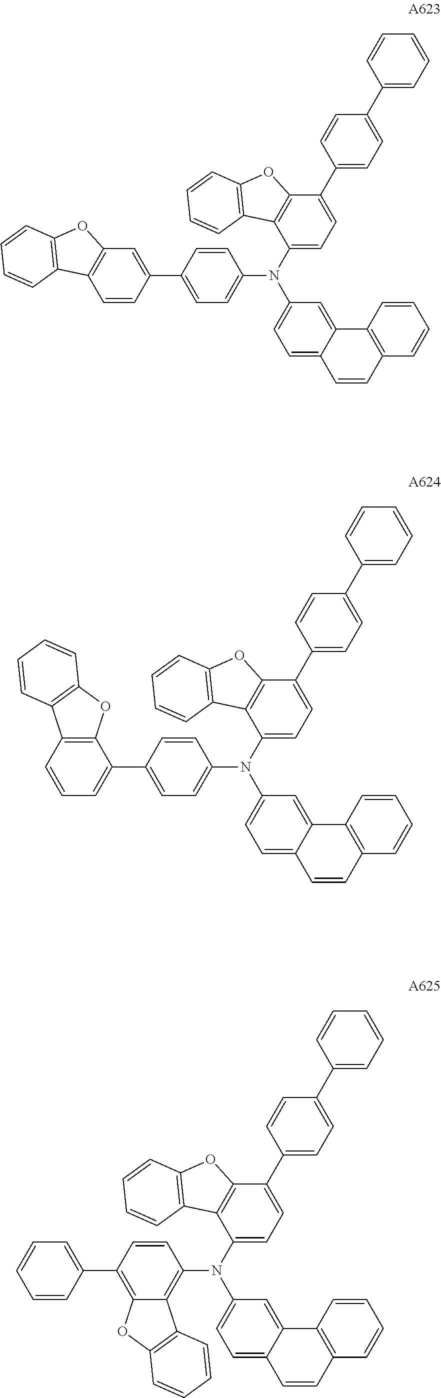 [SMILES: c1ccc(-c2ccc(-c3ccc(N(c4ccc(-c5ccc6c(c5)oc5ccccc56)cc4)c4ccc5ccc6ccccc6c5c4)c4c3oc3ccccc34)cc2)cc1 ; c1ccc(-c2ccc(-c3ccc(N(c4ccc(-c5cccc6c5oc5ccccc56)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccccc5)c5oc6ccccc6c45)c4c3oc3ccccc34)cc2)cc1] 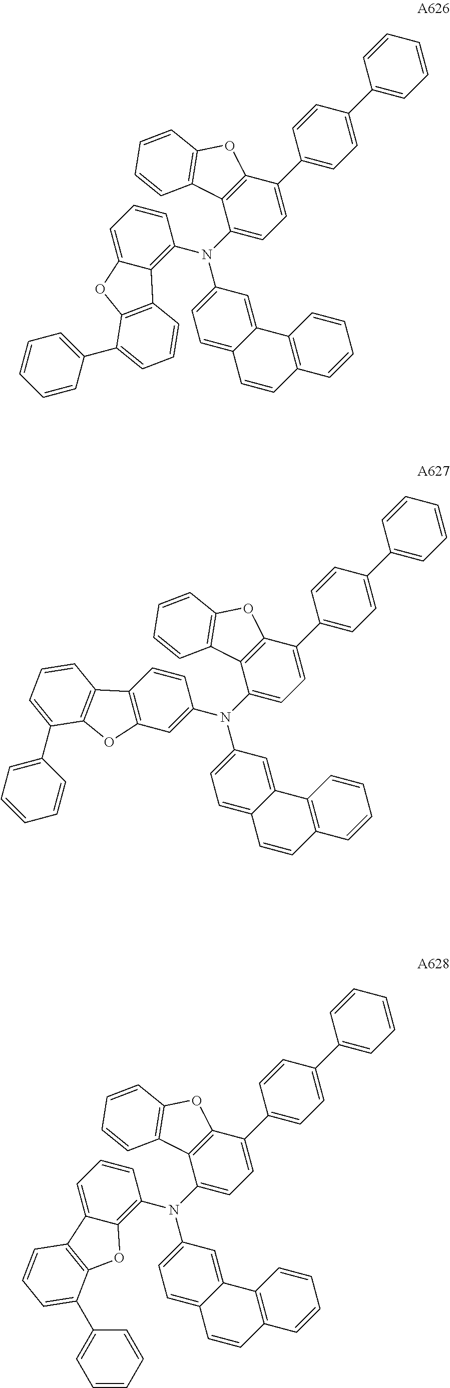 [SMILES: c1ccc(-c2ccc(-c3ccc(N(c4ccc5ccc6ccccc6c5c4)c4cccc5oc6c(-c7ccccc7)cccc6c45)c4c3oc3ccccc34)cc2)cc1 ; c1ccc(-c2ccc(-c3ccc(N(c4ccc5c(c4)oc4c(-c6ccccc6)cccc45)c4ccc5ccc6ccccc6c5c4)c4c3oc3ccccc34)cc2)cc1 ; c1ccc(-c2ccc(-c3ccc(N(c4ccc5ccc6ccccc6c5c4)c4cccc5c4oc4c(-c6ccccc6)cccc45)c4c3oc3ccccc34)cc2)cc1] 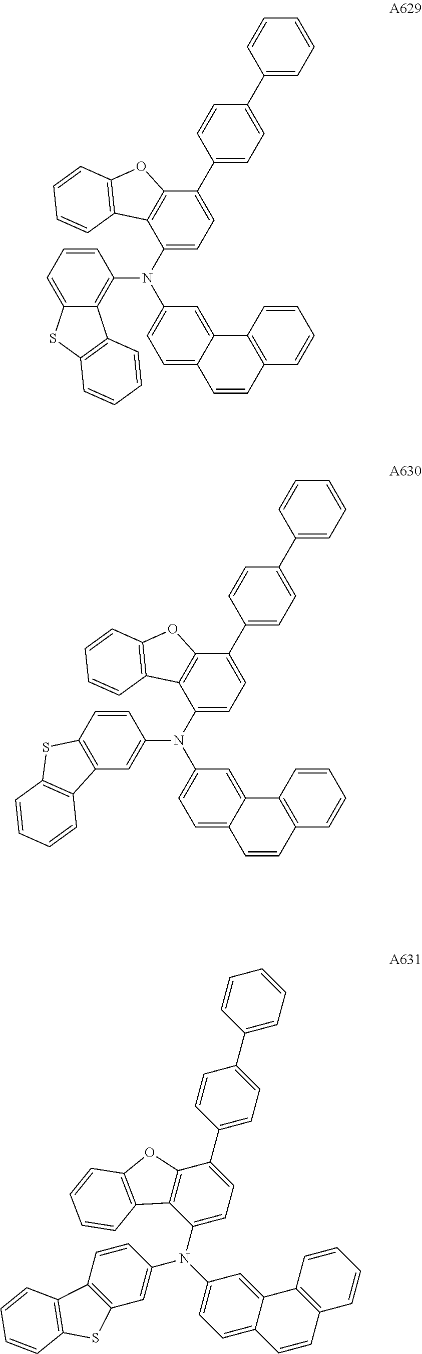 [SMILES: c1ccc(-c2ccc(-c3ccc(N(c4ccc5ccc6ccccc6c5c4)c4cccc5sc6ccccc6c45)c4c3oc3ccccc34)cc2)cc1 ; c1ccc(-c2ccc(-c3ccc(N(c4ccc5ccc6ccccc6c5c4)c4ccc5sc6ccccc6c5c4)c4c3oc3ccccc34)cc2)cc1 ; c1ccc(-c2ccc(-c3ccc(N(c4ccc5c(c4)sc4ccccc45)c4ccc5ccc6ccccc6c5c4)c4c3oc3ccccc34)cc2)cc1] 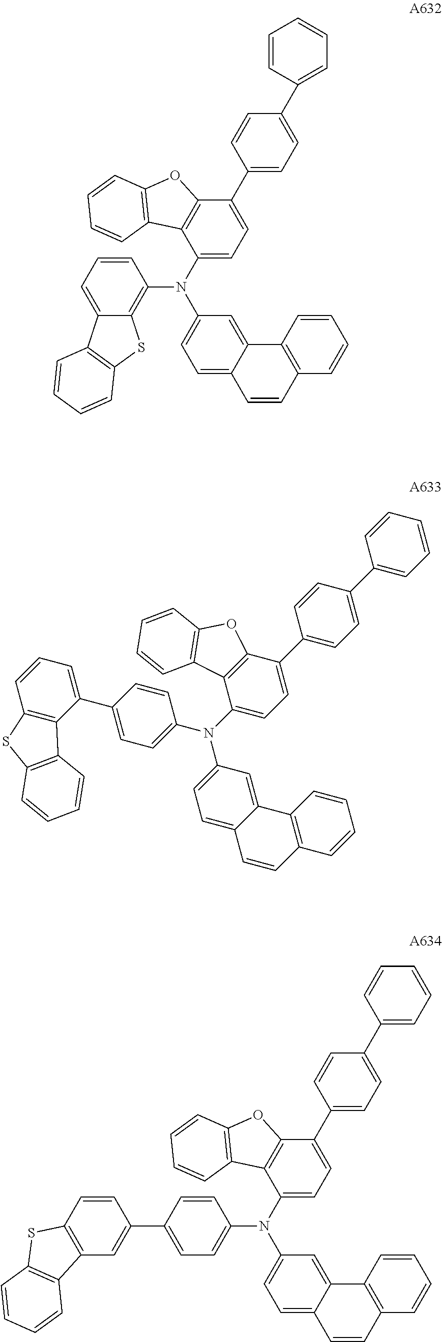 [SMILES: c1ccc(-c2ccc(-c3ccc(N(c4ccc5ccc6ccccc6c5c4)c4cccc5c4sc4ccccc45)c4c3oc3ccccc34)cc2)cc1 ; c1ccc(-c2ccc(-c3ccc(N(c4ccc(-c5cccc6sc7ccccc7c56)cc4)c4ccc5ccc6ccccc6c5c4)c4c3oc3ccccc34)cc2)cc1 ; c1ccc(-c2ccc(-c3ccc(N(c4ccc(-c5ccc6sc7ccccc7c6c5)cc4)c4ccc5ccc6ccccc6c5c4)c4c3oc3ccccc34)cc2)cc1] 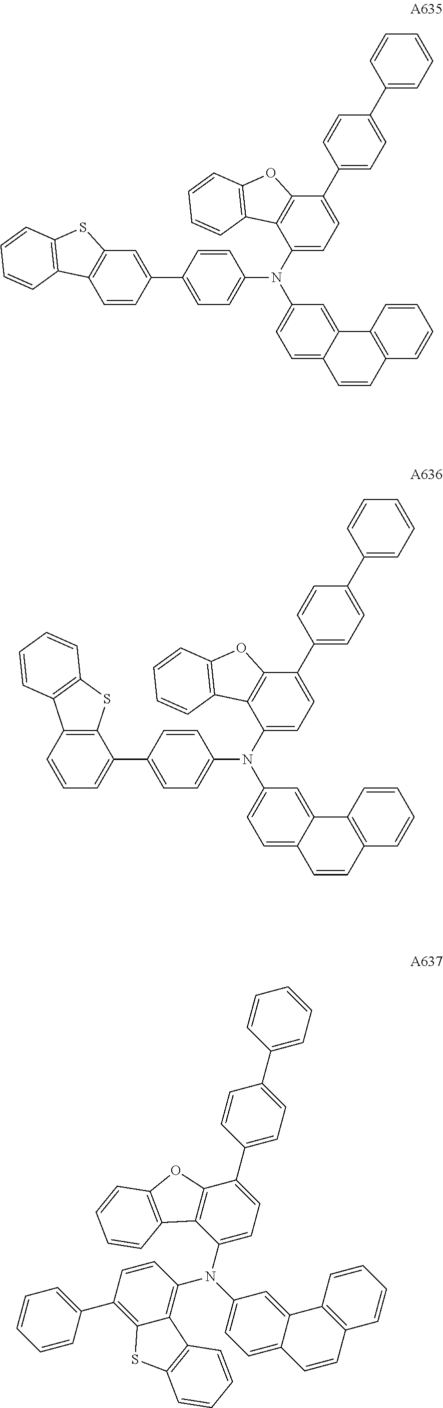 [SMILES: c1ccc(-c2ccc(-c3ccc(N(c4ccc(-c5ccc6c(c5)sc5ccccc56)cc4)c4ccc5ccc6ccccc6c5c4)c4c3oc3ccccc34)cc2)cc1 ; c1ccc(-c2ccc(-c3ccc(N(c4ccc(-c5cccc6c5sc5ccccc56)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc(-c5ccccc5)c5sc6ccccc6c45)c4c3oc3ccccc34)cc2)cc1] 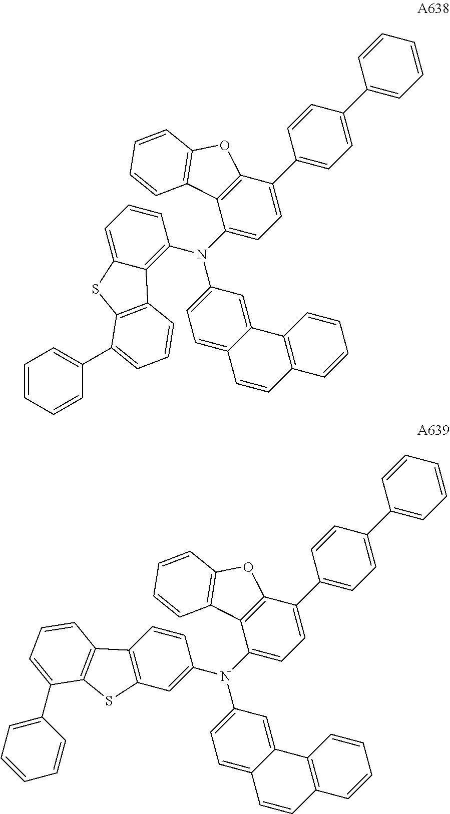 [SMILES: c1ccc(-c2ccc(-c3ccc(N(c4ccc5ccc6ccccc6c5c4)c4cccc5sc6c(-c7ccccc7)cccc6c45)c4c3oc3ccccc34)cc2)cc1 ; c1ccc(-c2ccc(-c3ccc(N(c4ccc5c(c4)sc4c(-c6ccccc6)cccc45)c4ccc5ccc6ccccc6c5c4)c4c3oc3ccccc34)cc2)cc1] 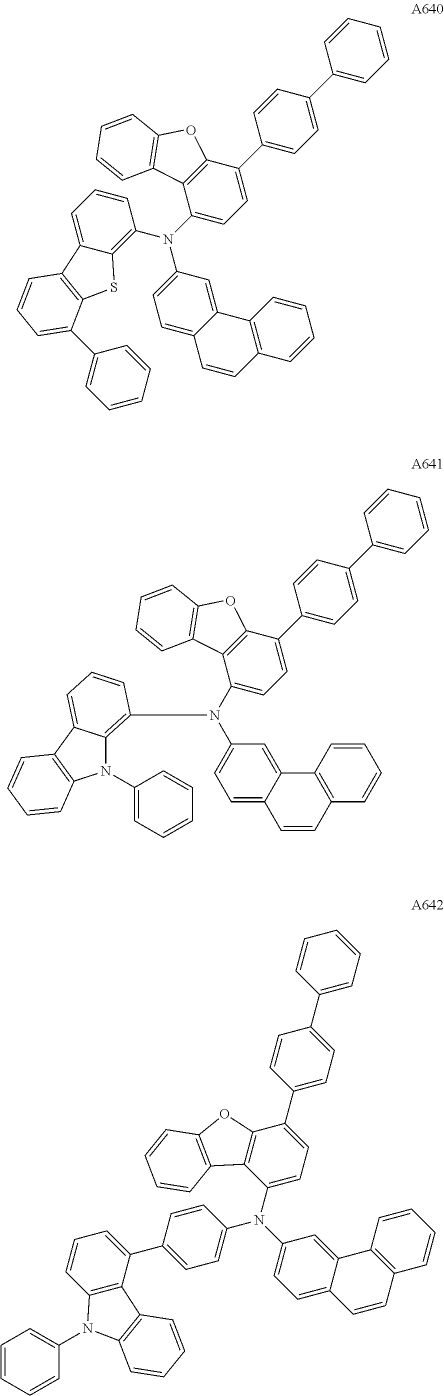 [SMILES: c1ccc(-c2ccc(-c3ccc(N(c4ccc5ccc6ccccc6c5c4)c4cccc5c4sc4c(-c6ccccc6)cccc45)c4c3oc3ccccc34)cc2)cc1 ; c1ccc(-c2ccc(-c3ccc(N(c4ccc5ccc6ccccc6c5c4)c4cccc5c6ccccc6n(-c6ccccc6)c45)c4c3oc3ccccc34)cc2)cc1 ; c1ccc(-c2ccc(-c3ccc(N(c4ccc(-c5cccc6c5c5ccccc5n6-c5ccccc5)cc4)c4ccc5ccc6ccccc6c5c4)c4c3oc3ccccc34)cc2)cc1] 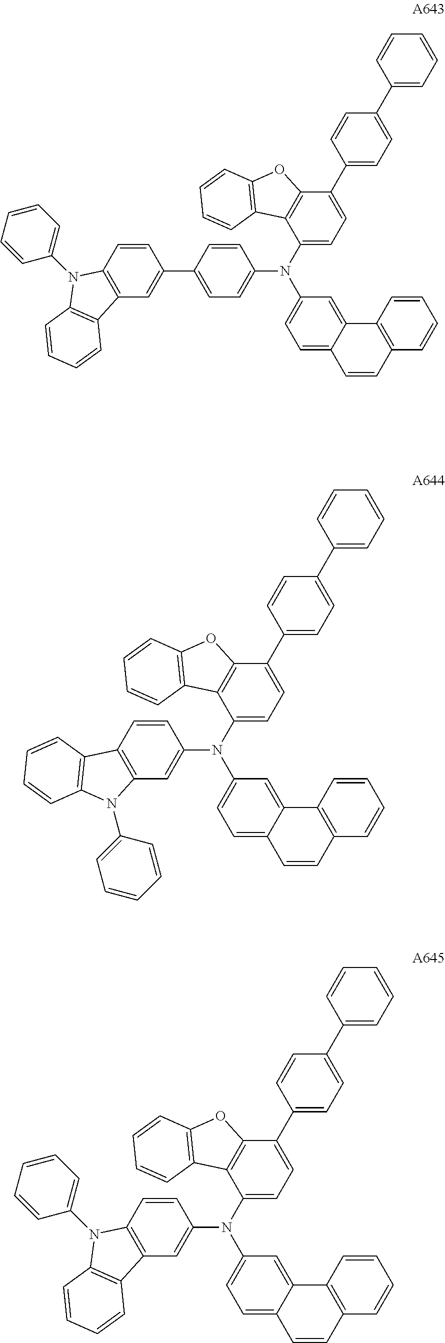 [SMILES: c1ccc(-c2ccc(-c3ccc(N(c4ccc(-c5ccc6c(c5)c5ccccc5n6-c5ccccc5)cc4)c4ccc5ccc6ccccc6c5c4)c4c3oc3ccccc34)cc2)cc1 ; c1ccc(-c2ccc(-c3ccc(N(c4ccc5ccc6ccccc6c5c4)c4ccc5c6ccccc6n(-c6ccccc6)c5c4)c4c3oc3ccccc34)cc2)cc1 ; c1ccc(-c2ccc(-c3ccc(N(c4ccc5ccc6ccccc6c5c4)c4ccc5c(c4)c4ccccc4n5-c4ccccc4)c4c3oc3ccccc34)cc2)cc1] 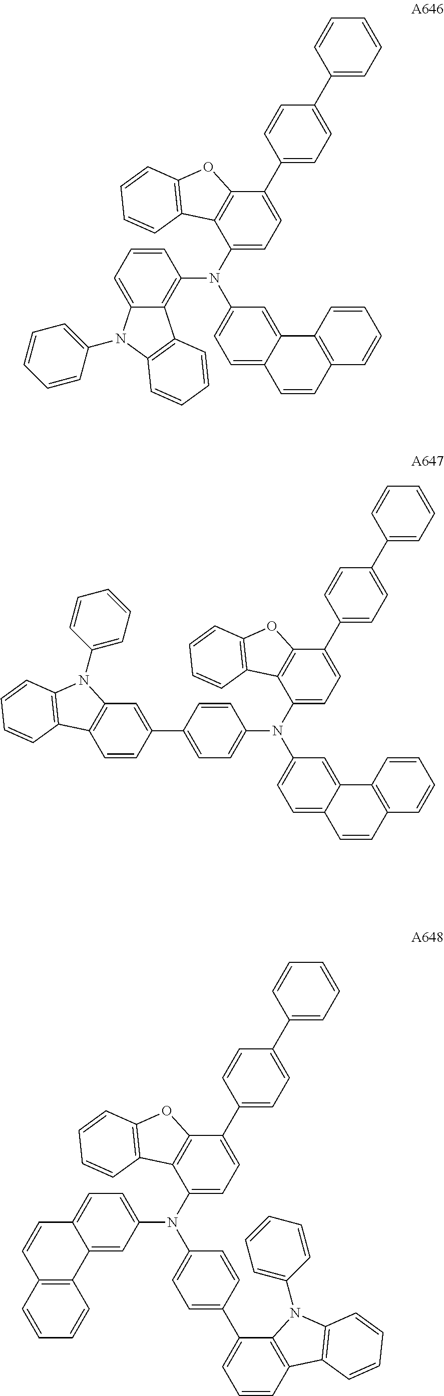 [SMILES: c1ccc(-c2ccc(-c3ccc(N(c4ccc5ccc6ccccc6c5c4)c4cccc5c4c4ccccc4n5-c4ccccc4)c4c3oc3ccccc34)cc2)cc1 ; c1ccc(-c2ccc(-c3ccc(N(c4ccc(-c5ccc6c7ccccc7n(-c7ccccc7)c6c5)cc4)c4ccc5ccc6ccccc6c5c4)c4c3oc3ccccc34)cc2)cc1 ; c1ccc(-c2ccc(-c3ccc(N(c4ccc(-c5cccc6c7ccccc7n(-c7ccccc7)c56)cc4)c4ccc5ccc6ccccc6c5c4)c4c3oc3ccccc34)cc2)cc1] 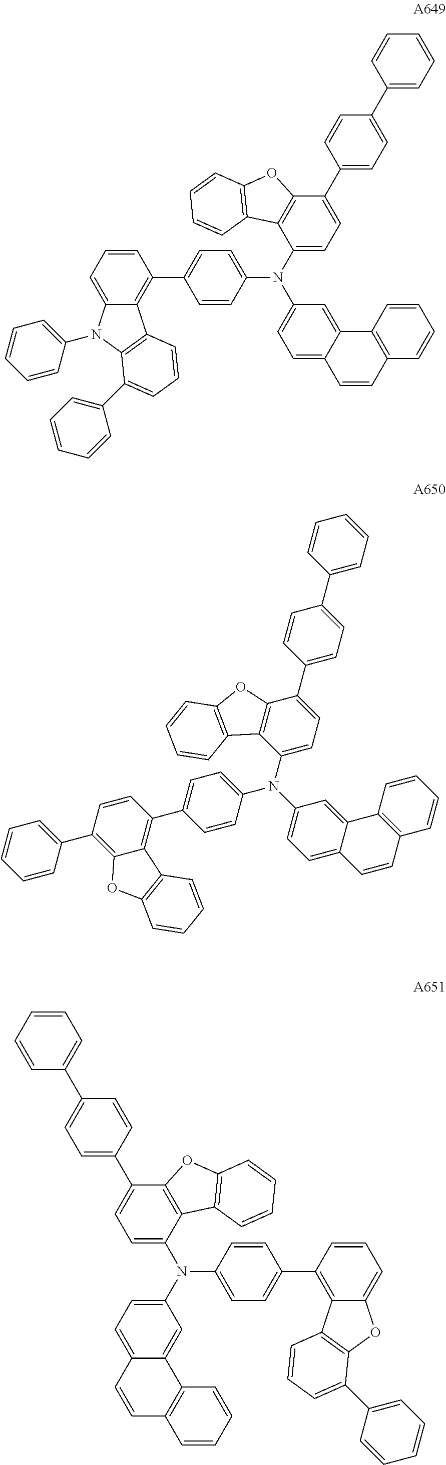 [SMILES: c1ccc(-c2ccc(-c3ccc(N(c4ccc(-c5cccc6c5c5cccc(-c7ccccc7)c5n6-c5ccccc5)cc4)c4ccc5ccc6ccccc6c5c4)c4c3oc3ccccc34)cc2)cc1 ; c1ccc(-c2ccc(-c3ccc(N(c4ccc(-c5ccc(-c6ccccc6)c6oc7ccccc7c56)cc4)c4ccc5ccc6ccccc6c5c4)c4c3oc3ccccc34)cc2)cc1 ; c1ccc(-c2ccc(-c3ccc(N(c4ccc(-c5cccc6oc7c(-c8ccccc8)cccc7c56)cc4)c4ccc5ccc6ccccc6c5c4)c4c3oc3ccccc34)cc2)cc1] 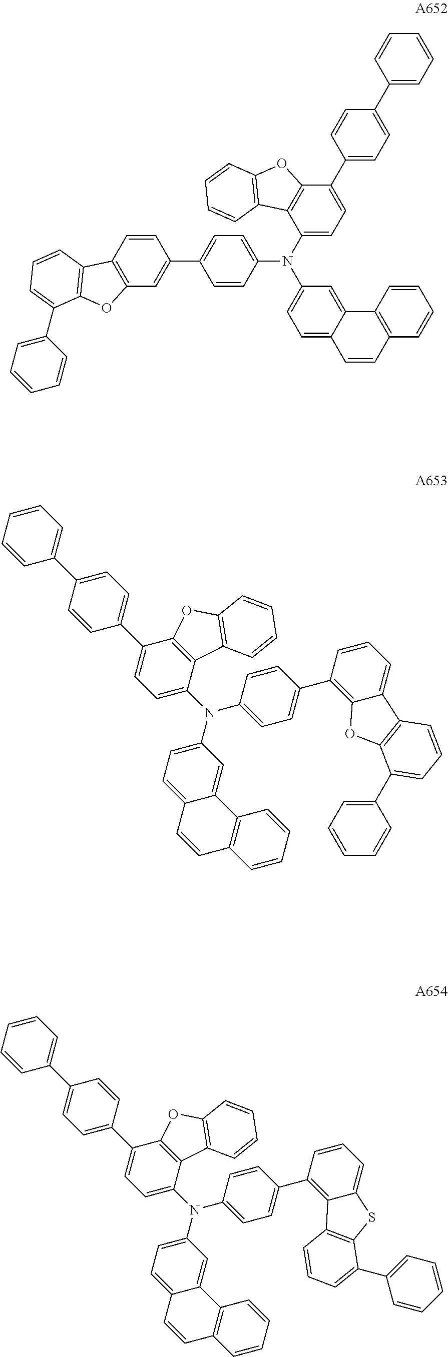 [SMILES: c1ccc(-c2ccc(-c3ccc(N(c4ccc(-c5ccc6c(c5)oc5c(-c7ccccc7)cccc56)cc4)c4ccc5ccc6ccccc6c5c4)c4c3oc3ccccc34)cc2)cc1 ; c1ccc(-c2ccc(-c3ccc(N(c4ccc(-c5cccc6c5oc5c(-c7ccccc7)cccc56)cc4)c4ccc5ccc6ccccc6c5c4)c4c3oc3ccccc34)cc2)cc1 ; c1ccc(-c2ccc(-c3ccc(N(c4ccc(-c5cccc6sc7c(-c8ccccc8)cccc7c56)cc4)c4ccc5ccc6ccccc6c5c4)c4c3oc3ccccc34)cc2)cc1] 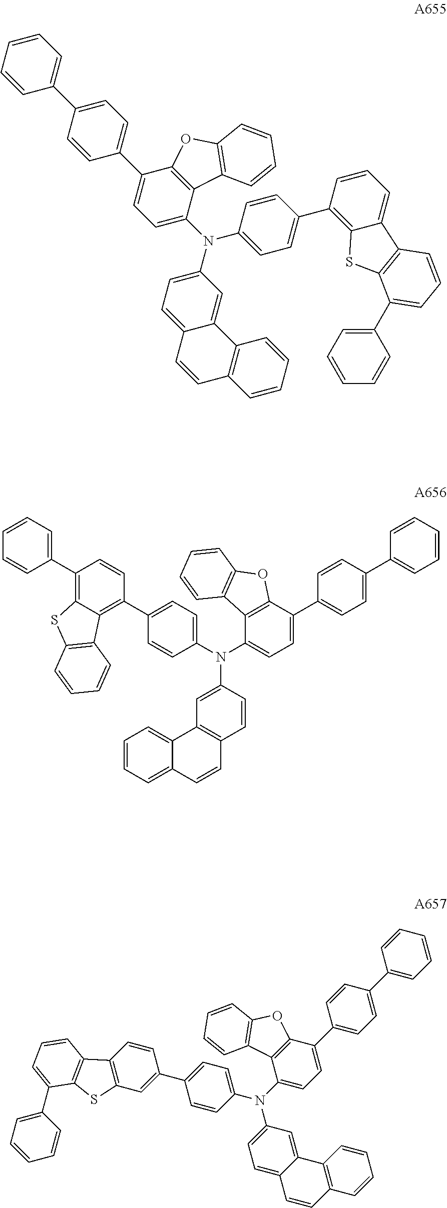 [SMILES: c1ccc(-c2ccc(-c3ccc(N(c4ccc(-c5cccc6c5sc5c(-c7ccccc7)cccc56)cc4)c4ccc5ccc6ccccc6c5c4)c4c3oc3ccccc34)cc2)cc1 ; c1ccc(-c2ccc(-c3ccc(N(c4ccc(-c5ccc(-c6ccccc6)c6sc7ccccc7c56)cc4)c4ccc5ccc6ccccc6c5c4)c4c3oc3ccccc34)cc2)cc1 ; c1ccc(-c2ccc(-c3ccc(N(c4ccc(-c5ccc6c(c5)sc5c(-c7ccccc7)cccc56)cc4)c4ccc5ccc6ccccc6c5c4)c4c3oc3ccccc34)cc2)cc1] 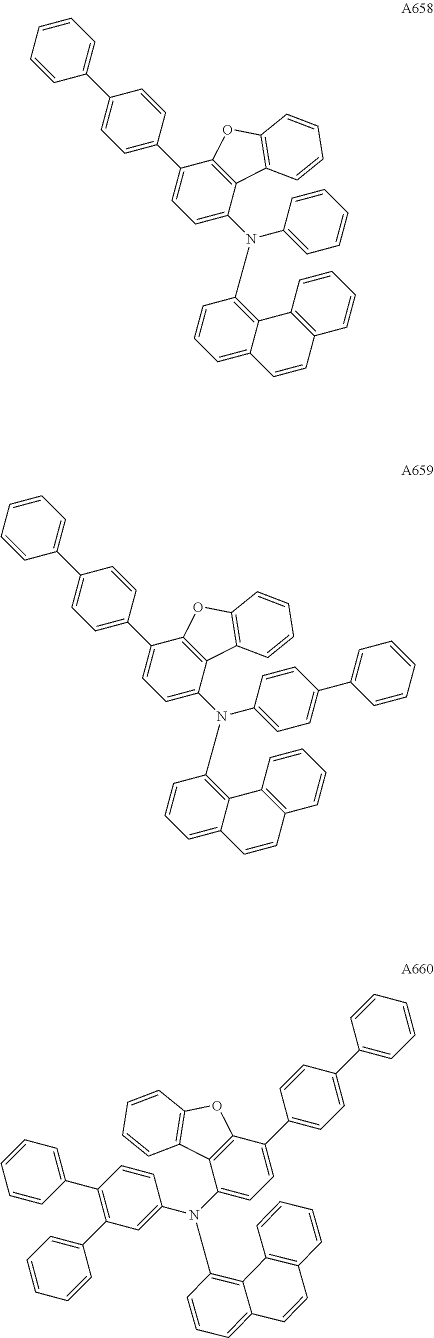 [SMILES: c1ccc(-c2ccc(-c3ccc(N(c4ccccc4)c4cccc5ccc6ccccc6c45)c4c3oc3ccccc34)cc2)cc1 ; c1ccc(-c2ccc(-c3ccc(N(c4ccc(-c5ccccc5)cc4)c4cccc5ccc6ccccc6c45)c4c3oc3ccccc34)cc2)cc1 ; c1ccc(-c2ccc(-c3ccc(N(c4ccc(-c5ccccc5)c(-c5ccccc5)c4)c4cccc5ccc6ccccc6c45)c4c3oc3ccccc34)cc2)cc1] 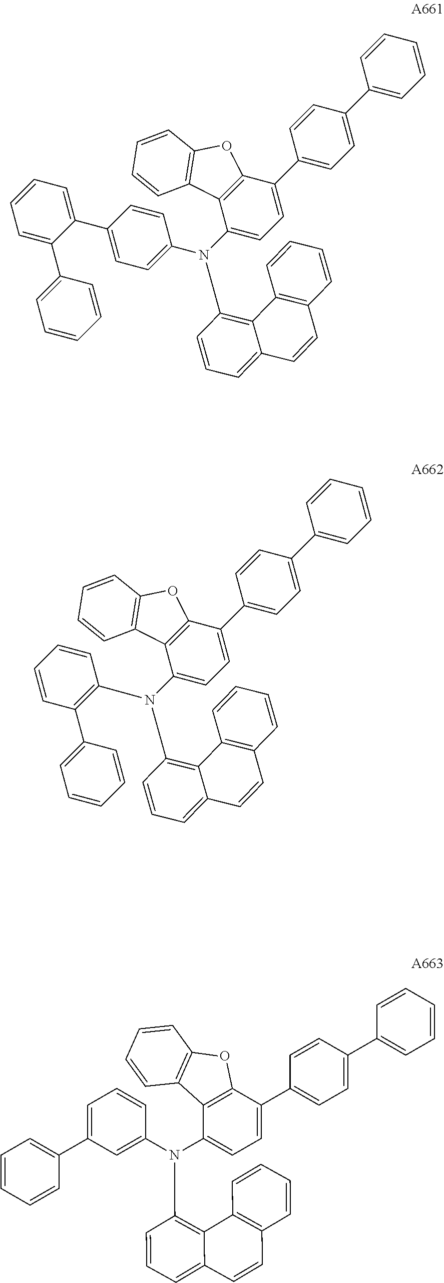 [SMILES: c1ccc(-c2ccc(-c3ccc(N(c4ccc(-c5ccccc5-c5ccccc5)cc4)c4cccc5ccc6ccccc6c45)c4c3oc3ccccc34)cc2)cc1 ; c1ccc(-c2ccc(-c3ccc(N(c4ccccc4-c4ccccc4)c4cccc5ccc6ccccc6c45)c4c3oc3ccccc34)cc2)cc1 ; c1ccc(-c2ccc(-c3ccc(N(c4cccc(-c5ccccc5)c4)c4cccc5ccc6ccccc6c45)c4c3oc3ccccc34)cc2)cc1] 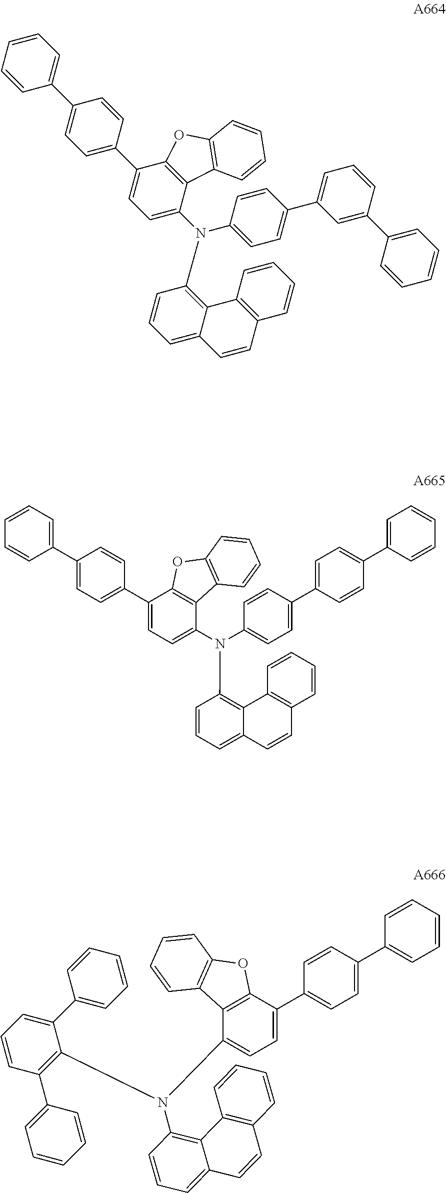 [SMILES: c1ccc(-c2ccc(-c3ccc(N(c4ccc(-c5cccc(-c6ccccc6)c5)cc4)c4cccc5ccc6ccccc6c45)c4c3oc3ccccc34)cc2)cc1 ; c1ccc(-c2ccc(-c3ccc(N(c4cccc5ccc6ccccc6c45)c4ccc(-c5ccc(-c6ccccc6)cc5)c5oc6ccccc6c45)cc3)cc2)cc1 ; c1ccc(-c2ccc(-c3ccc(N(c4c(-c5ccccc5)cccc4-c4ccccc4)c4cccc5ccc6ccccc6c45)c4c3oc3ccccc34)cc2)cc1] 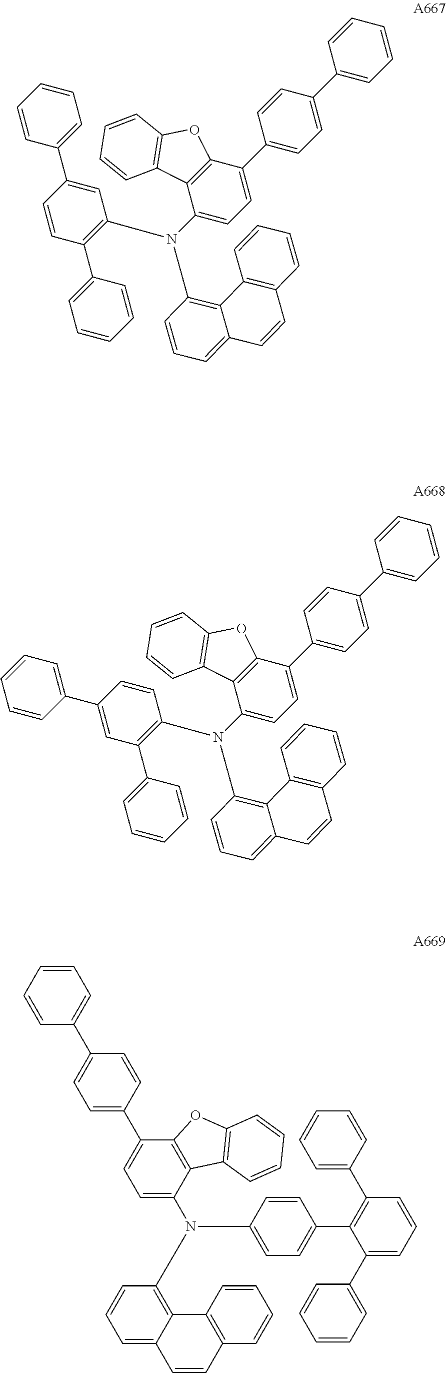 [SMILES: c1ccc(-c2ccc(-c3ccc(N(c4cc(-c5ccccc5)ccc4-c4ccccc4)c4cccc5ccc6ccccc6c45)c4c3oc3ccccc34)cc2)cc1 ; c1ccc(-c2ccc(-c3ccc(N(c4ccc(-c5ccccc5)cc4-c4ccccc4)c4cccc5ccc6ccccc6c45)c4c3oc3ccccc34)cc2)cc1 ; c1ccc(-c2ccc(-c3ccc(N(c4ccc(-c5c(-c6ccccc6)cccc5-c5ccccc5)cc4)c4cccc5ccc6ccccc6c45)c4c3oc3ccccc34)cc2)cc1] 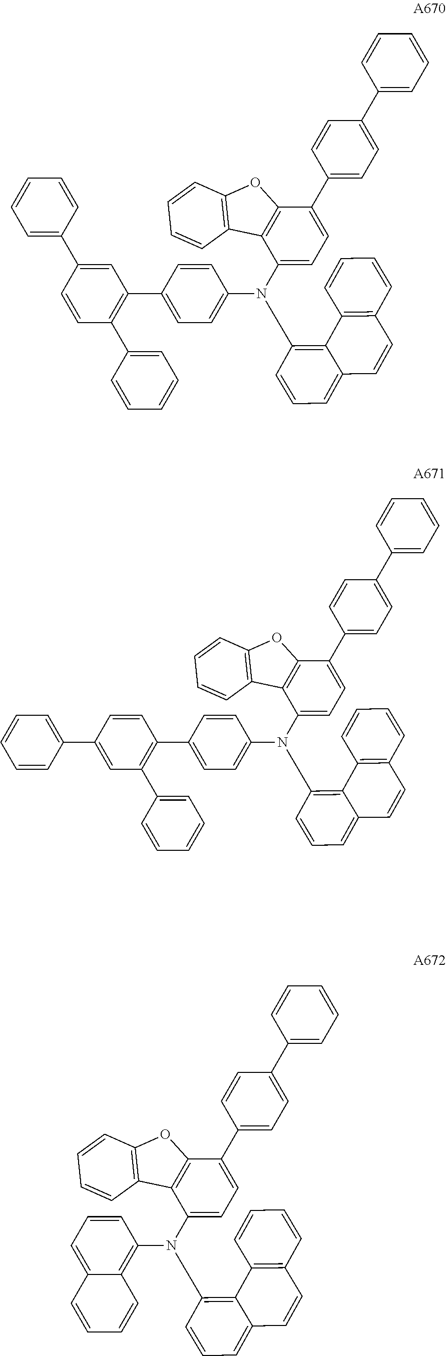 [SMILES: c1ccc(-c2ccc(-c3ccc(N(c4ccc(-c5cc(-c6ccccc6)ccc5-c5ccccc5)cc4)c4cccc5ccc6ccccc6c45)c4c3oc3ccccc34)cc2)cc1 ; c1ccc(-c2ccc(-c3ccc(N(c4ccc(-c5ccc(-c6ccccc6)cc5-c5ccccc5)cc4)c4cccc5ccc6ccccc6c45)c4c3oc3ccccc34)cc2)cc1 ; c1ccc(-c2ccc(-c3ccc(N(c4cccc5ccccc45)c4cccc5ccc6ccccc6c45)c4c3oc3ccccc34)cc2)cc1] 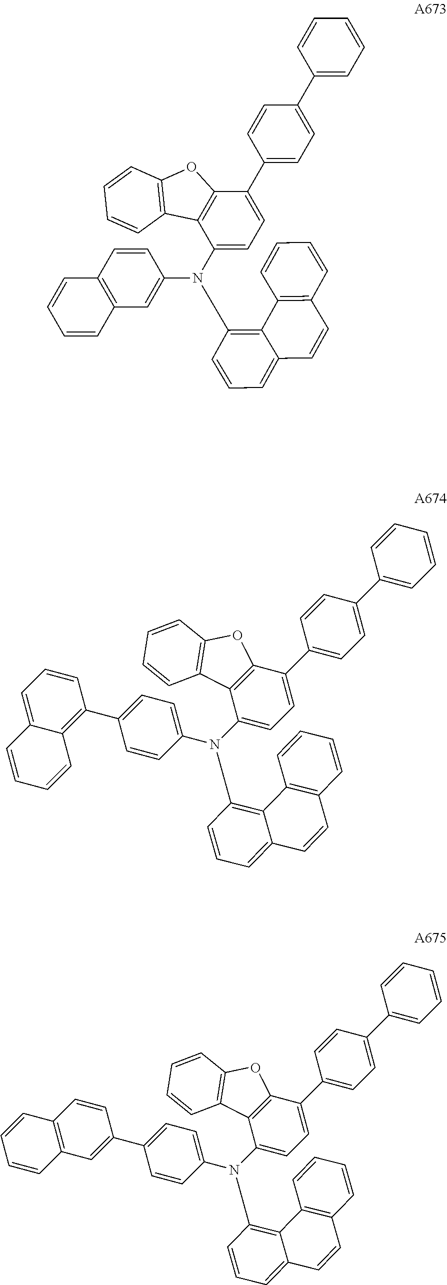 [SMILES: c1ccc(-c2ccc(-c3ccc(N(c4ccc5ccccc5c4)c4cccc5ccc6ccccc6c45)c4c3oc3ccccc34)cc2)cc1 ; c1ccc(-c2ccc(-c3ccc(N(c4ccc(-c5cccc6ccccc56)cc4)c4cccc5ccc6ccccc6c45)c4c3oc3ccccc34)cc2)cc1 ; c1ccc(-c2ccc(-c3ccc(N(c4ccc(-c5ccc6ccccc6c5)cc4)c4cccc5ccc6ccccc6c45)c4c3oc3ccccc34)cc2)cc1] 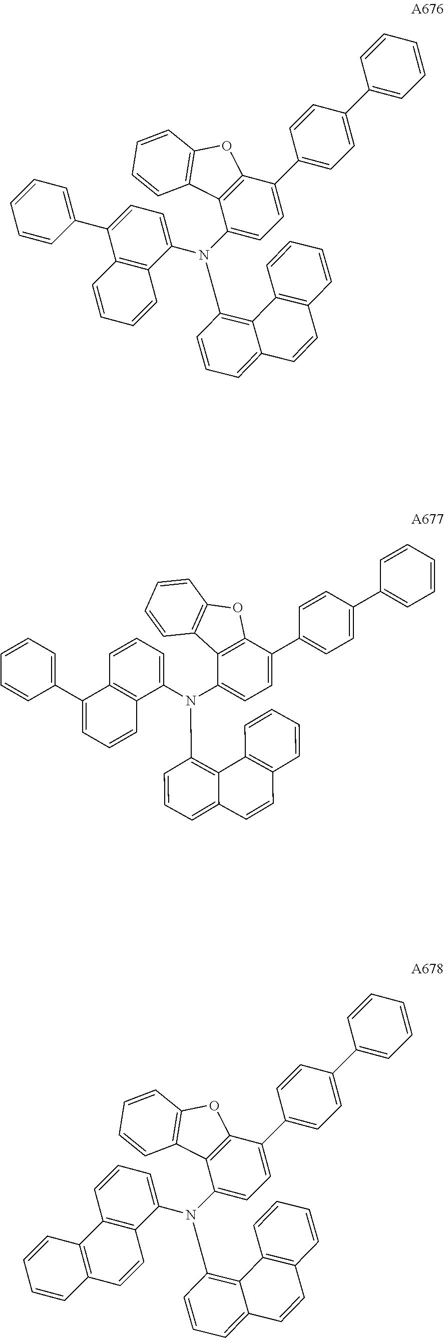 [SMILES: c1ccc(-c2ccc(-c3ccc(N(c4ccc(-c5ccccc5)c5ccccc45)c4cccc5ccc6ccccc6c45)c4c3oc3ccccc34)cc2)cc1 ; c1ccc(-c2ccc(-c3ccc(N(c4cccc5c(-c6ccccc6)cccc45)c4cccc5ccc6ccccc6c45)c4c3oc3ccccc34)cc2)cc1 ; c1ccc(-c2ccc(-c3ccc(N(c4cccc5c4ccc4ccccc45)c4cccc5ccc6ccccc6c45)c4c3oc3ccccc34)cc2)cc1] 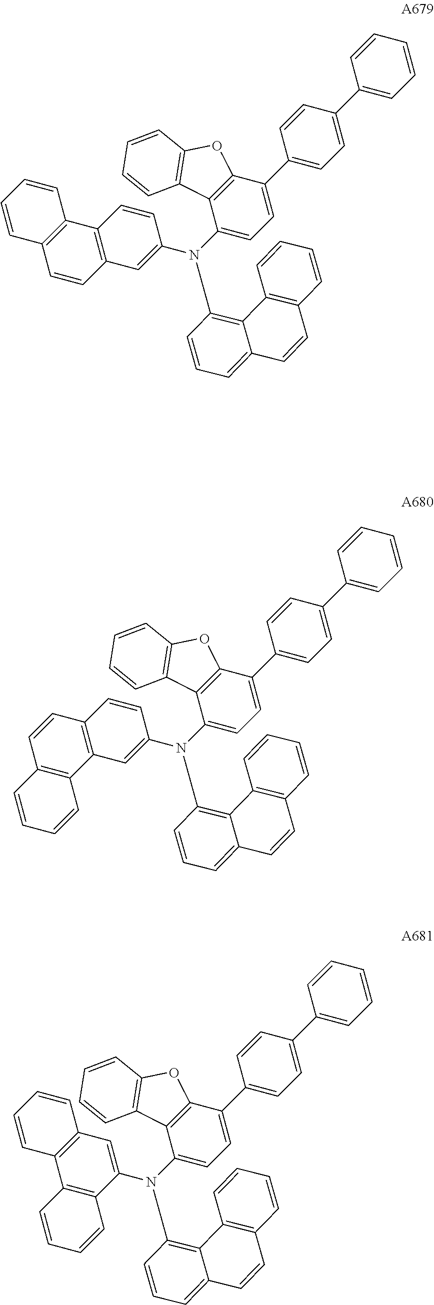 [SMILES: c1ccc(-c2ccc(-c3ccc(N(c4ccc5c(ccc6ccccc65)c4)c4cccc5ccc6ccccc6c45)c4c3oc3ccccc34)cc2)cc1 ; c1ccc(-c2ccc(-c3ccc(N(c4ccc5ccc6ccccc6c5c4)c4cccc5ccc6ccccc6c45)c4c3oc3ccccc34)cc2)cc1 ; c1ccc(-c2ccc(-c3ccc(N(c4cc5ccccc5c5ccccc45)c4cccc5ccc6ccccc6c45)c4c3oc3ccccc34)cc2)cc1] 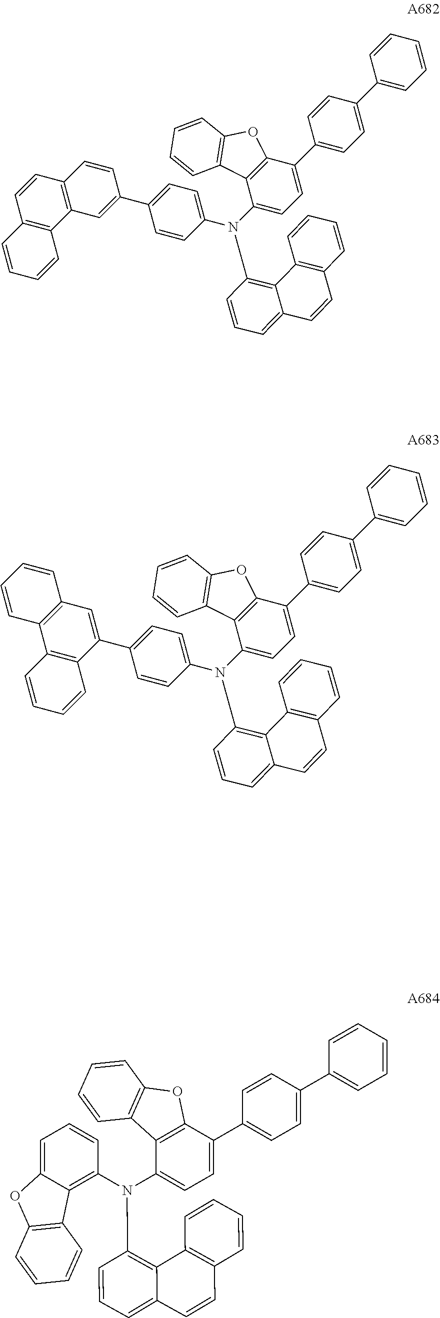 [SMILES: c1ccc(-c2ccc(-c3ccc(N(c4ccc(-c5ccc6ccc7ccccc7c6c5)cc4)c4cccc5ccc6ccccc6c45)c4c3oc3ccccc34)cc2)cc1 ; c1ccc(-c2ccc(-c3ccc(N(c4ccc(-c5cc6ccccc6c6ccccc56)cc4)c4cccc5ccc6ccccc6c45)c4c3oc3ccccc34)cc2)cc1 ; c1ccc(-c2ccc(-c3ccc(N(c4cccc5ccc6ccccc6c45)c4cccc5oc6ccccc6c45)c4c3oc3ccccc34)cc2)cc1] 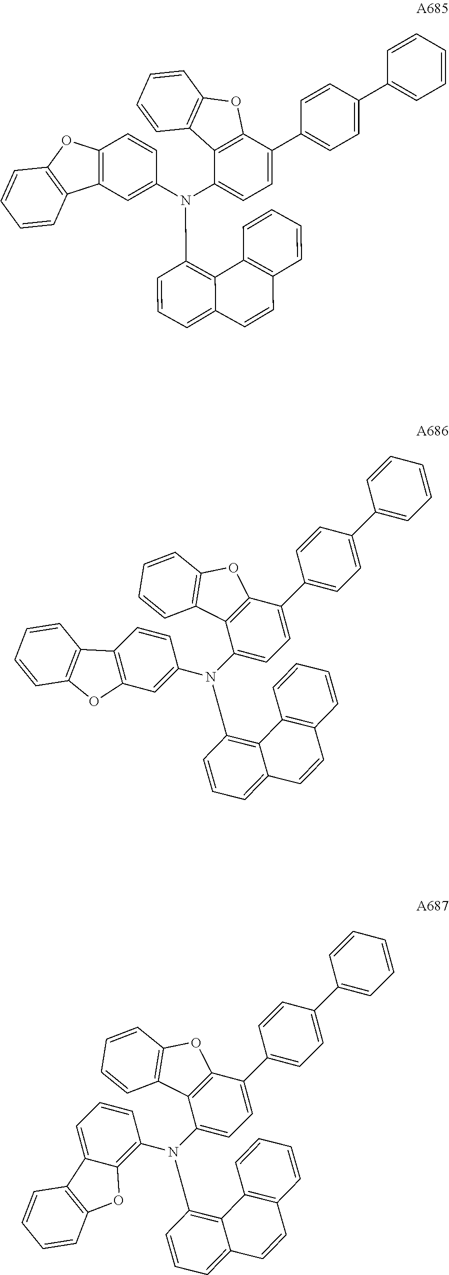 [SMILES: c1ccc(-c2ccc(-c3ccc(N(c4ccc5oc6ccccc6c5c4)c4cccc5ccc6ccccc6c45)c4c3oc3ccccc34)cc2)cc1 ; c1ccc(-c2ccc(-c3ccc(N(c4ccc5c(c4)oc4ccccc45)c4cccc5ccc6ccccc6c45)c4c3oc3ccccc34)cc2)cc1 ; c1ccc(-c2ccc(-c3ccc(N(c4cccc5c4oc4ccccc45)c4cccc5ccc6ccccc6c45)c4c3oc3ccccc34)cc2)cc1] 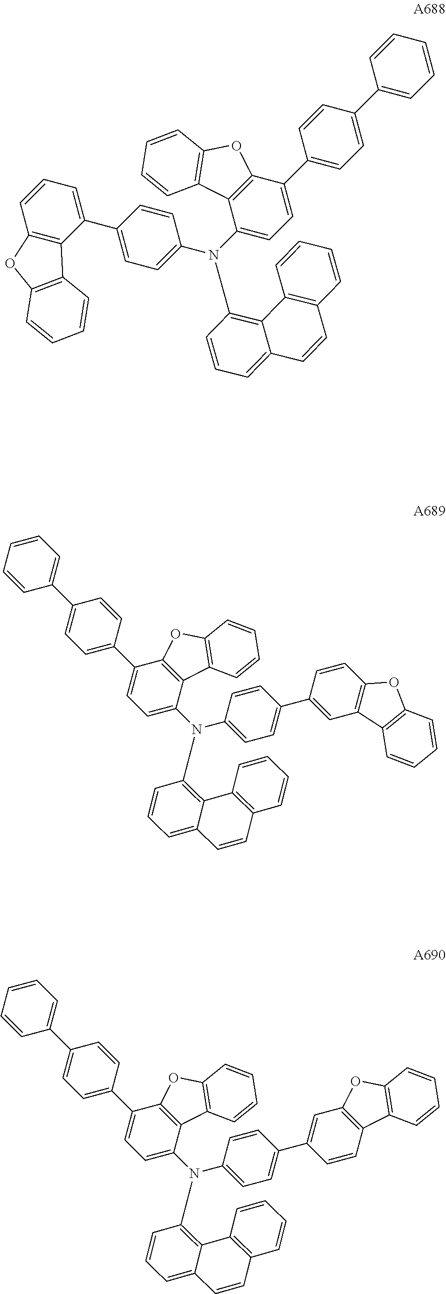 [SMILES: c1ccc(-c2ccc(-c3ccc(N(c4ccc(-c5cccc6oc7ccccc7c56)cc4)c4cccc5ccc6ccccc6c45)c4c3oc3ccccc34)cc2)cc1 ; c1ccc(-c2ccc(-c3ccc(N(c4ccc(-c5ccc6oc7ccccc7c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)oc5ccccc56)cc4)c4cccc5ccc6ccccc6c45)c4c3oc3ccccc34)cc2)cc1] 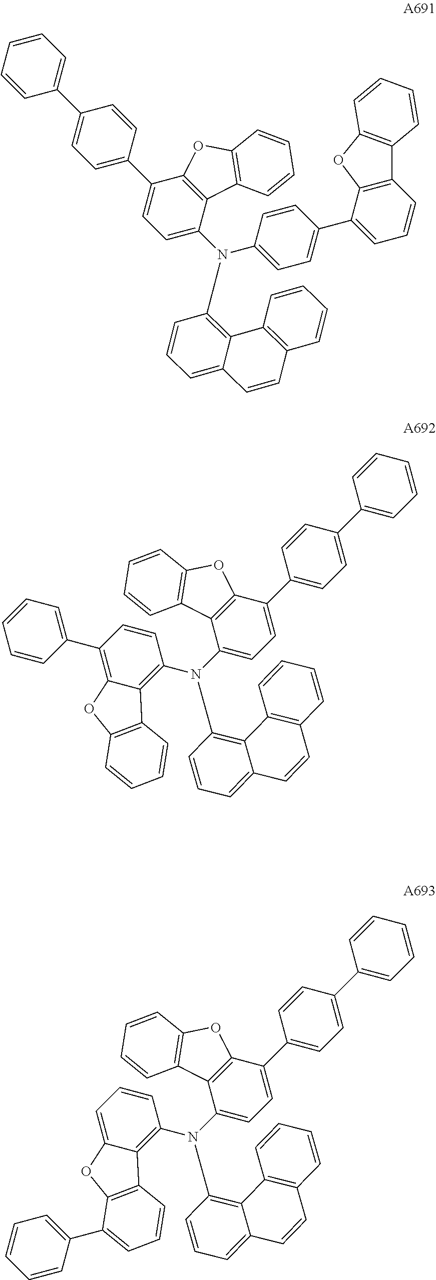 [SMILES: c1ccc(-c2ccc(-c3ccc(N(c4ccc(-c5cccc6c5oc5ccccc56)cc4)c4cccc5ccc6ccccc6c45)c4c3oc3ccccc34)cc2)cc1 ; c1ccc(-c2ccc(-c3ccc(N(c4cccc5ccc6ccccc6c45)c4ccc(-c5ccccc5)c5oc6ccccc6c45)c4c3oc3ccccc34)cc2)cc1 ; c1ccc(-c2ccc(-c3ccc(N(c4cccc5ccc6ccccc6c45)c4cccc5oc6c(-c7ccccc7)cccc6c45)c4c3oc3ccccc34)cc2)cc1] 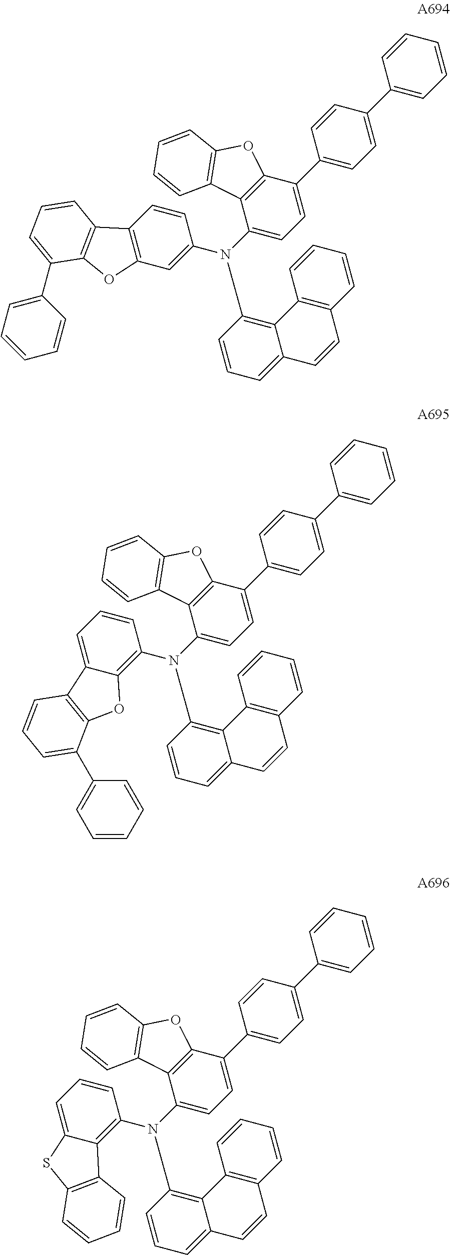 [SMILES: c1ccc(-c2ccc(-c3ccc(N(c4ccc5c(c4)oc4c(-c6ccccc6)cccc45)c4cccc5ccc6ccccc6c45)c4c3oc3ccccc34)cc2)cc1 ; c1ccc(-c2ccc(-c3ccc(N(c4cccc5c4oc4c(-c6ccccc6)cccc45)c4cccc5ccc6ccccc6c45)c4c3oc3ccccc34)cc2)cc1 ; c1ccc(-c2ccc(-c3ccc(N(c4cccc5ccc6ccccc6c45)c4cccc5sc6ccccc6c45)c4c3oc3ccccc34)cc2)cc1] 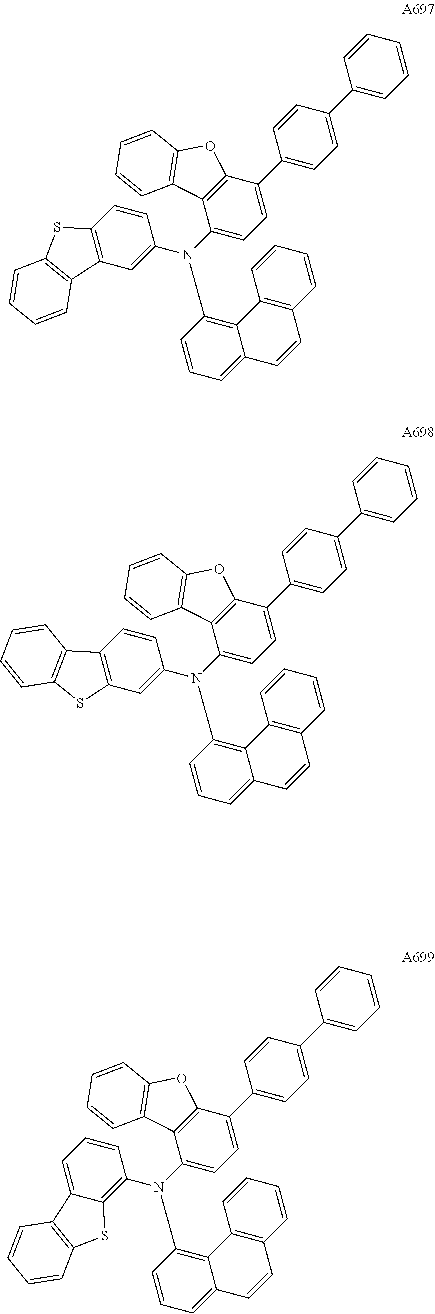 [SMILES: c1ccc(-c2ccc(-c3ccc(N(c4ccc5sc6ccccc6c5c4)c4cccc5ccc6ccccc6c45)c4c3oc3ccccc34)cc2)cc1 ; c1ccc(-c2ccc(-c3ccc(N(c4ccc5c(c4)sc4ccccc45)c4cccc5ccc6ccccc6c45)c4c3oc3ccccc34)cc2)cc1 ; c1ccc(-c2ccc(-c3ccc(N(c4cccc5c4sc4ccccc45)c4cccc5ccc6ccccc6c45)c4c3oc3ccccc34)cc2)cc1] 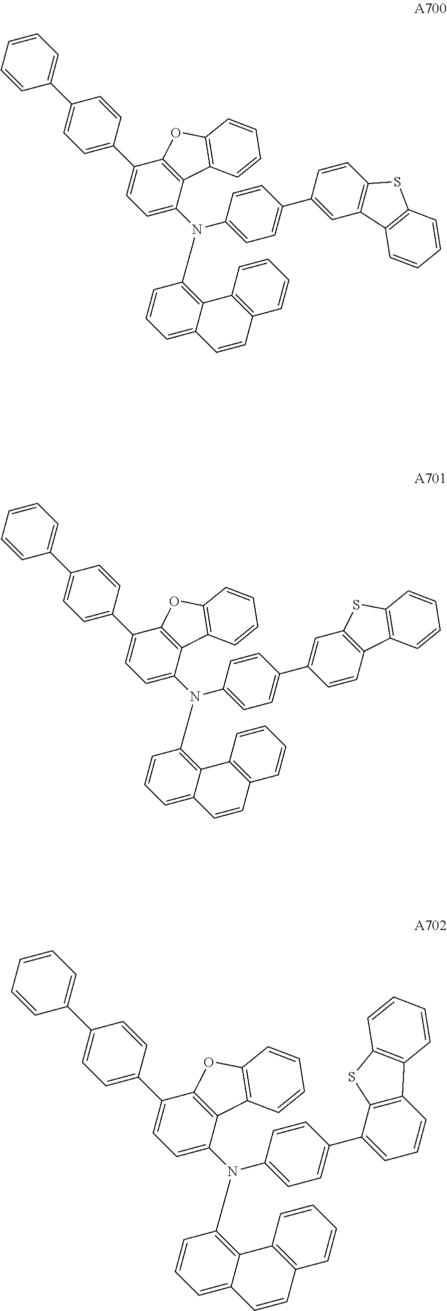 [SMILES: c1ccc(-c2ccc(-c3ccc(N(c4ccc(-c5ccc6sc7ccccc7c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)sc5ccccc56)cc4)c4cccc5ccc6ccccc6c45)c4c3oc3ccccc34)cc2)cc1 ; c1ccc(-c2ccc(-c3ccc(N(c4ccc(-c5cccc6c5sc5ccccc56)cc4)c4cccc5ccc6ccccc6c45)c4c3oc3ccccc34)cc2)cc1] 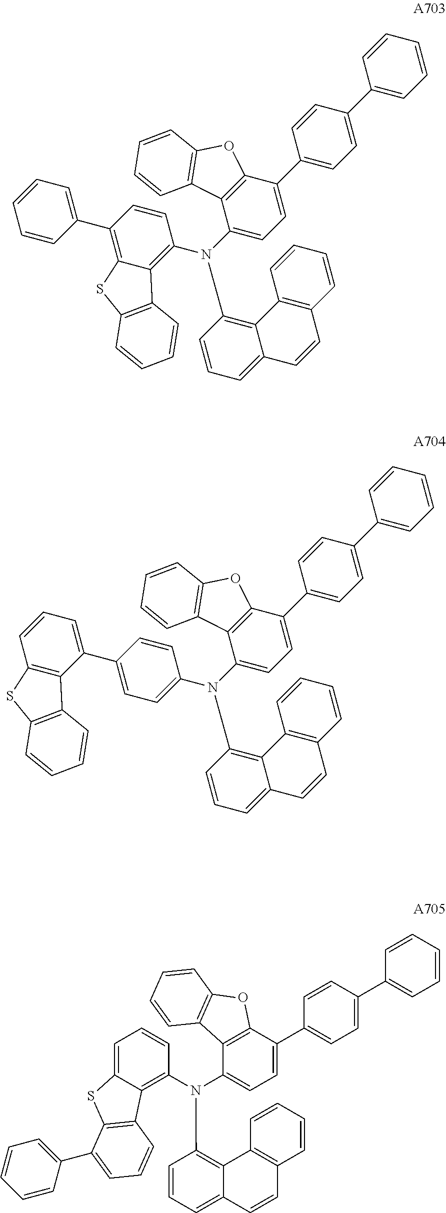 [SMILES: c1ccc(-c2ccc(-c3ccc(N(c4cccc5ccc6ccccc6c45)c4ccc(-c5ccccc5)c5sc6ccccc6c45)c4c3oc3ccccc34)cc2)cc1 ; c1ccc(-c2ccc(-c3ccc(N(c4ccc(-c5cccc6sc7ccccc7c56)cc4)c4cccc5ccc6ccccc6c45)c4c3oc3ccccc34)cc2)cc1 ; c1ccc(-c2ccc(-c3ccc(N(c4cccc5ccc6ccccc6c45)c4cccc5sc6c(-c7ccccc7)cccc6c45)c4c3oc3ccccc34)cc2)cc1] 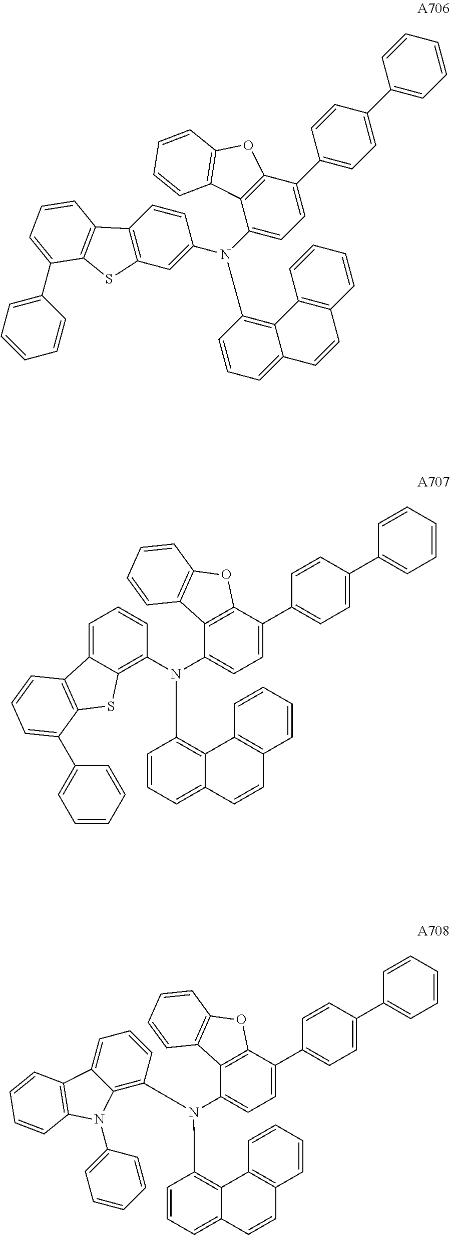 [SMILES: c1ccc(-c2ccc(-c3ccc(N(c4ccc5c(c4)sc4c(-c6ccccc6)cccc45)c4cccc5ccc6ccccc6c45)c4c3oc3ccccc34)cc2)cc1 ; c1ccc(-c2ccc(-c3ccc(N(c4cccc5c4sc4c(-c6ccccc6)cccc45)c4cccc5ccc6ccccc6c45)c4c3oc3ccccc34)cc2)cc1 ; c1ccc(-c2ccc(-c3ccc(N(c4cccc5ccc6ccccc6c45)c4cccc5c6ccccc6n(-c6ccccc6)c45)c4c3oc3ccccc34)cc2)cc1] 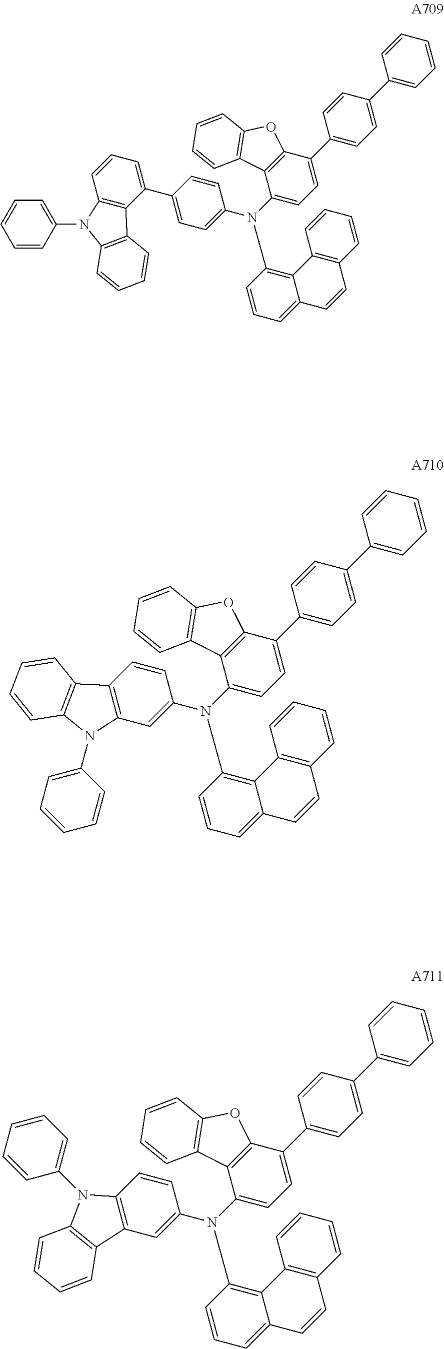 [SMILES: c1ccc(-c2ccc(-c3ccc(N(c4ccc(-c5cccc6c5c5ccccc5n6-c5ccccc5)cc4)c4cccc5ccc6ccccc6c45)c4c3oc3ccccc34)cc2)cc1 ; c1ccc(-c2ccc(-c3ccc(N(c4ccc5c6ccccc6n(-c6ccccc6)c5c4)c4cccc5ccc6ccccc6c45)c4c3oc3ccccc34)cc2)cc1 ; c1ccc(-c2ccc(-c3ccc(N(c4ccc5c(c4)c4ccccc4n5-c4ccccc4)c4cccc5ccc6ccccc6c45)c4c3oc3ccccc34)cc2)cc1] 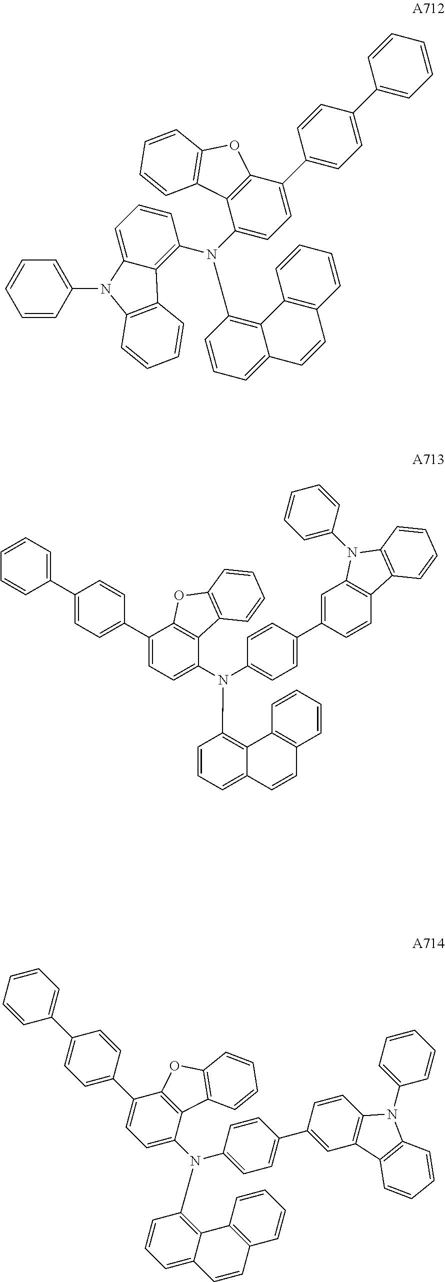 [SMILES: c1ccc(-c2ccc(-c3ccc(N(c4cccc5ccc6ccccc6c45)c4cccc5c4c4ccccc4n5-c4ccccc4)c4c3oc3ccccc34)cc2)cc1 ; c1ccc(-c2ccc(-c3ccc(N(c4ccc(-c5ccc6c7ccccc7n(-c7ccccc7)c6c5)cc4)c4cccc5ccc6ccccc6c45)c4c3oc3ccccc34)cc2)cc1 ; c1ccc(-c2ccc(-c3ccc(N(c4ccc(-c5ccc6c(c5)c5ccccc5n6-c5ccccc5)cc4)c4cccc5ccc6ccccc6c45)c4c3oc3ccccc34)cc2)cc1] 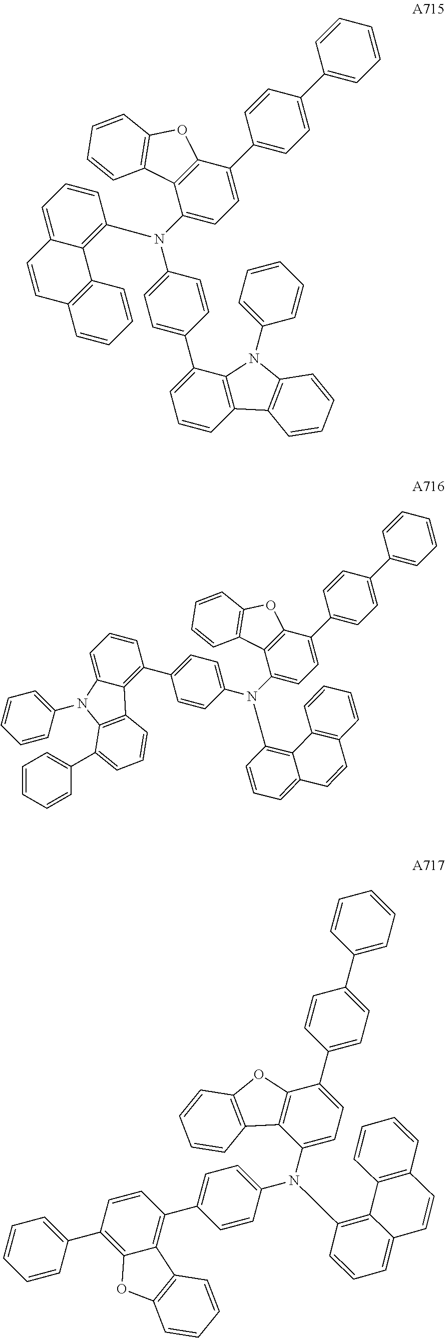 [SMILES: c1ccc(-c2ccc(-c3ccc(N(c4ccc(-c5cccc6c7ccccc7n(-c7ccccc7)c56)cc4)c4cccc5ccc6ccccc6c45)c4c3oc3ccccc34)cc2)cc1 ; c1ccc(-c2ccc(-c3ccc(N(c4ccc(-c5cccc6c5c5cccc(-c7ccccc7)c5n6-c5ccccc5)cc4)c4cccc5ccc6ccccc6c45)c4c3oc3ccccc34)cc2)cc1 ; c1ccc(-c2ccc(-c3ccc(N(c4ccc(-c5ccc(-c6ccccc6)c6oc7ccccc7c56)cc4)c4cccc5ccc6ccccc6c45)c4c3oc3ccccc34)cc2)cc1] 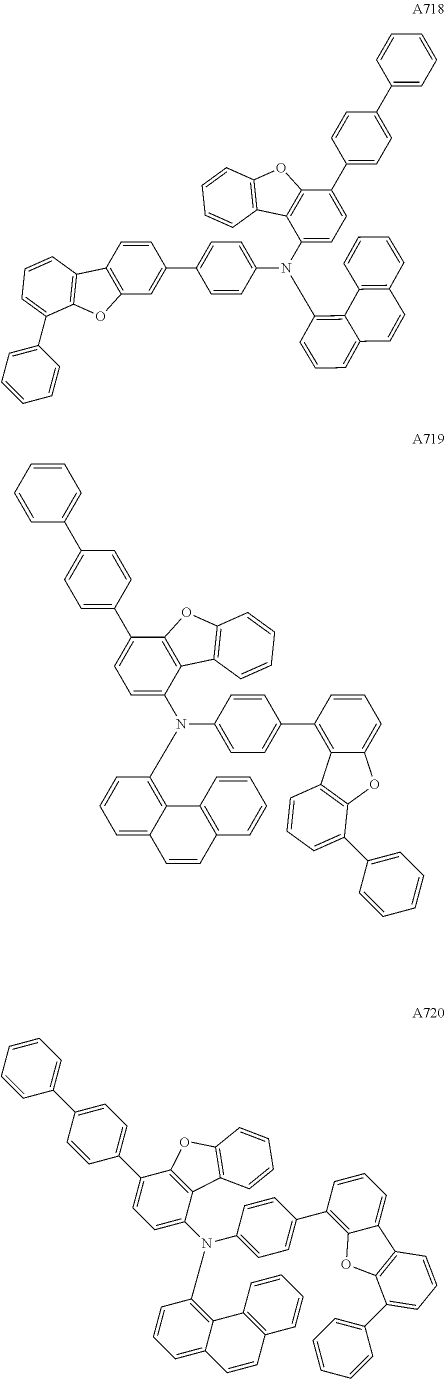 [SMILES: c1ccc(-c2ccc(-c3ccc(N(c4ccc(-c5ccc6c(c5)oc5c(-c7ccccc7)cccc56)cc4)c4cccc5ccc6ccccc6c45)c4c3oc3ccccc34)cc2)cc1 ; c1ccc(-c2ccc(-c3ccc(N(c4ccc(-c5cccc6oc7c(-c8ccccc8)cccc7c56)cc4)c4cccc5ccc6ccccc6c45)c4c3oc3ccccc34)cc2)cc1 ; c1ccc(-c2ccc(-c3ccc(N(c4ccc(-c5cccc6c5oc5c(-c7ccccc7)cccc56)cc4)c4cccc5ccc6ccccc6c45)c4c3oc3ccccc34)cc2)cc1] 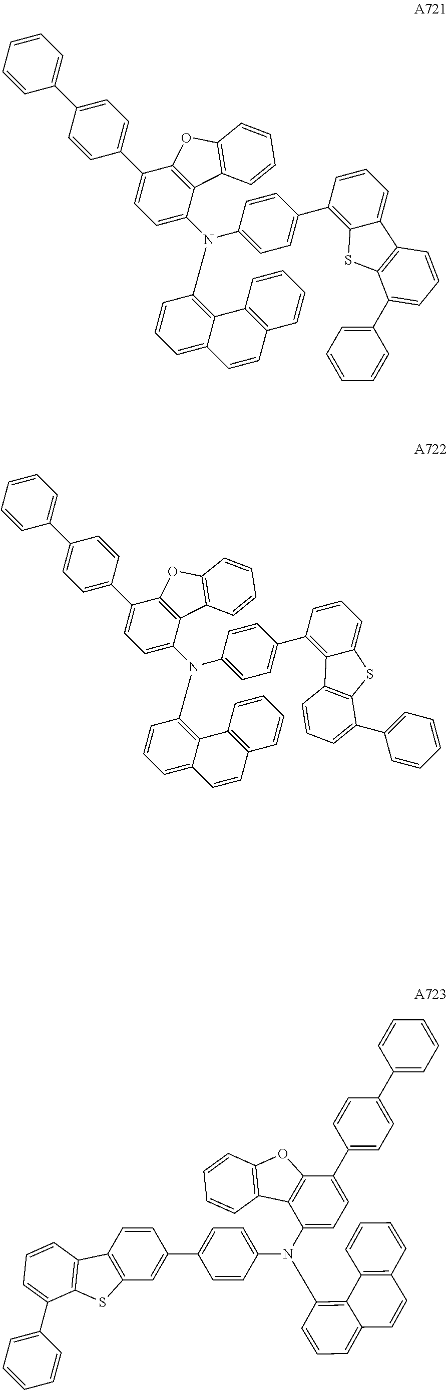 [SMILES: c1ccc(-c2ccc(-c3ccc(N(c4ccc(-c5cccc6c5sc5c(-c7ccccc7)cccc56)cc4)c4cccc5ccc6ccccc6c45)c4c3oc3ccccc34)cc2)cc1 ; c1ccc(-c2ccc(-c3ccc(N(c4ccc(-c5cccc6sc7c(-c8ccccc8)cccc7c56)cc4)c4cccc5ccc6ccccc6c45)c4c3oc3ccccc34)cc2)cc1 ; c1ccc(-c2ccc(-c3ccc(N(c4ccc(-c5ccc6c(c5)sc5c(-c7ccccc7)cccc56)cc4)c4cccc5ccc6ccccc6c45)c4c3oc3ccccc34)cc2)cc1] 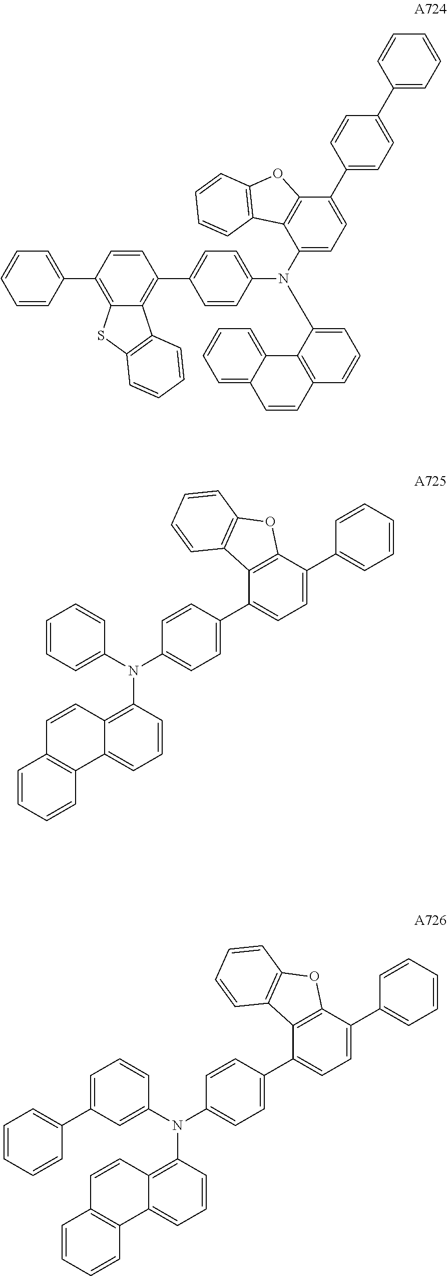 [SMILES: c1ccc(-c2ccc(-c3ccc(N(c4ccc(-c5ccc(-c6ccccc6)c6sc7ccccc7c56)cc4)c4cccc5ccc6ccccc6c45)c4c3oc3ccccc34)cc2)cc1 ; c1ccc(-c2ccc(-c3ccc(N(c4ccccc4)c4cccc5c4ccc4ccccc45)cc3)c3c2oc2ccccc23)cc1 ; c1ccc(-c2cccc(N(c3ccc(-c4ccc(-c5ccccc5)c5oc6ccccc6c45)cc3)c3cccc4c3ccc3ccccc34)c2)cc1] 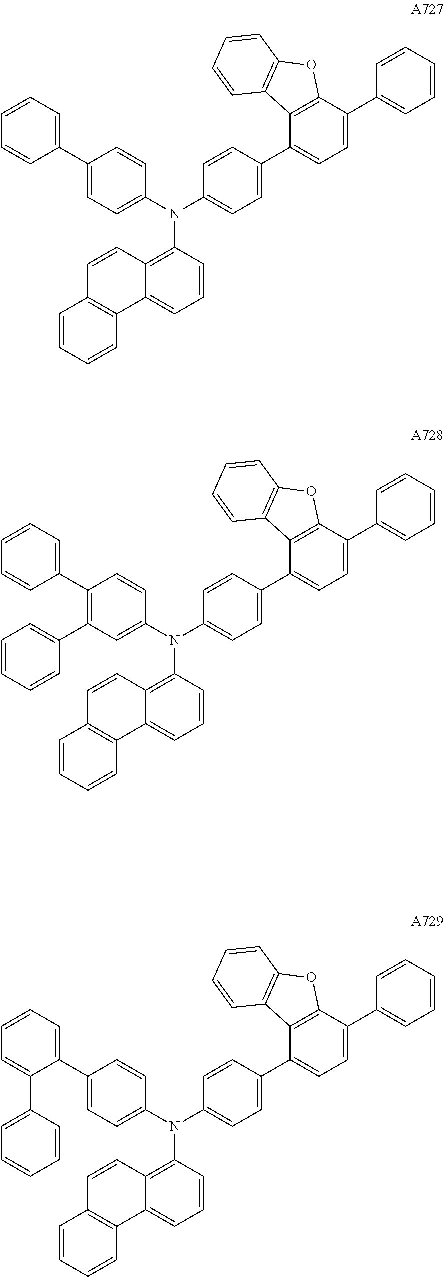 [SMILES: c1ccc(-c2ccc(N(c3ccc(-c4ccc(-c5ccccc5)c5oc6ccccc6c45)cc3)c3cccc4c3ccc3ccccc34)cc2)cc1 ; c1ccc(-c2ccc(N(c3ccc(-c4ccc(-c5ccccc5)c5oc6ccccc6c45)cc3)c3cccc4c3ccc3ccccc34)cc2-c2ccccc2)cc1 ; c1ccc(-c2ccccc2-c2ccc(N(c3ccc(-c4ccc(-c5ccccc5)c5oc6ccccc6c45)cc3)c3cccc4c3ccc3ccccc34)cc2)cc1] 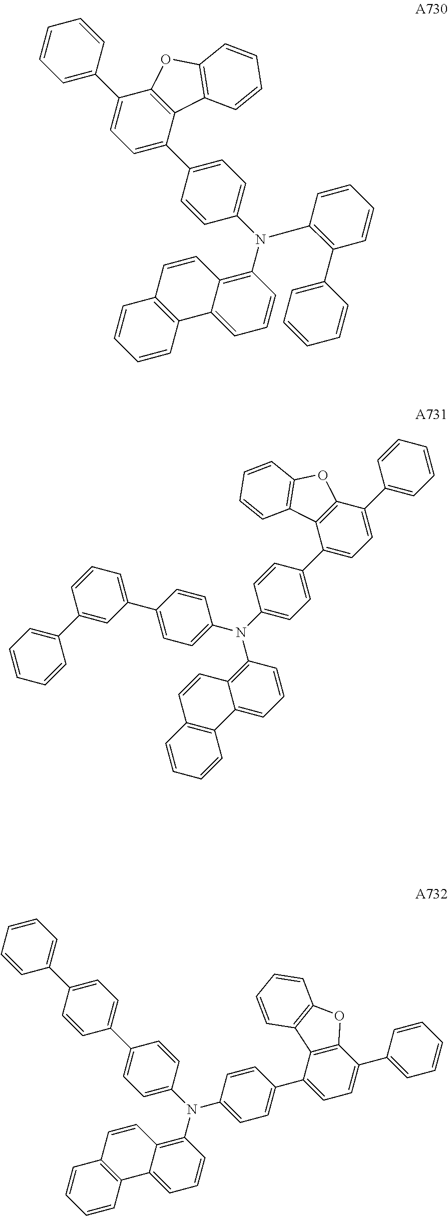 [SMILES: c1ccc(-c2ccccc2N(c2ccc(-c3ccc(-c4ccccc4)c4oc5ccccc5c34)cc2)c2cccc3c2ccc2ccccc23)cc1 ; c1ccc(-c2cccc(-c3ccc(N(c4ccc(-c5ccc(-c6ccccc6)c6oc7ccccc7c56)cc4)c4cccc5c4ccc4ccccc45)cc3)c2)cc1 ; c1ccc(-c2ccc(-c3ccc(N(c4ccc(-c5ccc(-c6ccccc6)c6oc7ccccc7c56)cc4)c4cccc5c4ccc4ccccc45)cc3)cc2)cc1] 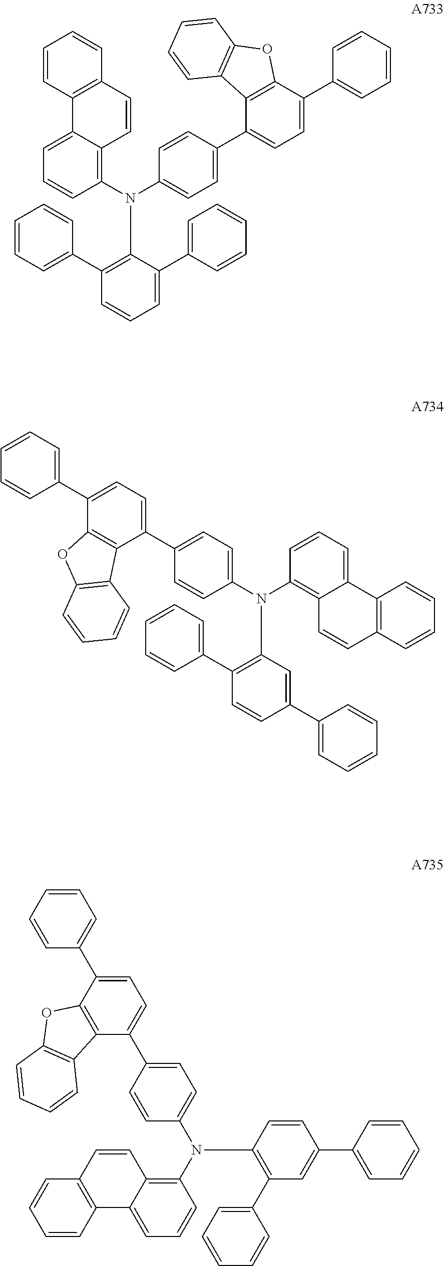 [SMILES: c1ccc(-c2cccc(-c3ccccc3)c2N(c2ccc(-c3ccc(-c4ccccc4)c4oc5ccccc5c34)cc2)c2cccc3c2ccc2ccccc23)cc1 ; c1ccc(-c2ccc(-c3ccccc3)c(N(c3ccc(-c4ccc(-c5ccccc5)c5oc6ccccc6c45)cc3)c3cccc4c3ccc3ccccc34)c2)cc1 ; c1ccc(-c2ccc(N(c3ccc(-c4ccc(-c5ccccc5)c5oc6ccccc6c45)cc3)c3cccc4c3ccc3ccccc34)c(-c3ccccc3)c2)cc1] 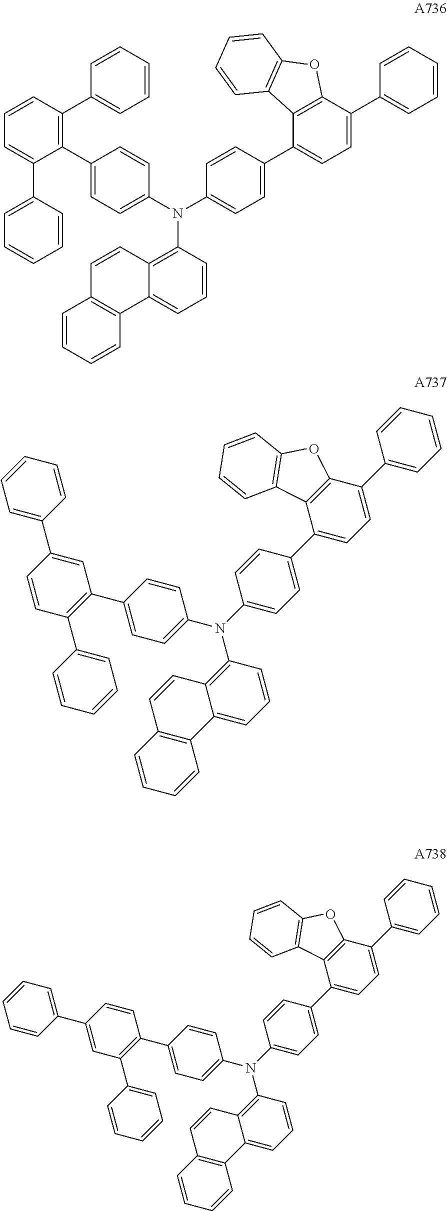 [SMILES: c1ccc(-c2cccc(-c3ccccc3)c2-c2ccc(N(c3ccc(-c4ccc(-c5ccccc5)c5oc6ccccc6c45)cc3)c3cccc4c3ccc3ccccc34)cc2)cc1 ; c1ccc(-c2ccc(-c3ccccc3)c(-c3ccc(N(c4ccc(-c5ccc(-c6ccccc6)c6oc7ccccc7c56)cc4)c4cccc5c4ccc4ccccc45)cc3)c2)cc1 ; c1ccc(-c2ccc(-c3ccc(N(c4ccc(-c5ccc(-c6ccccc6)c6oc7ccccc7c56)cc4)c4cccc5c4ccc4ccccc45)cc3)c(-c3ccccc3)c2)cc1] 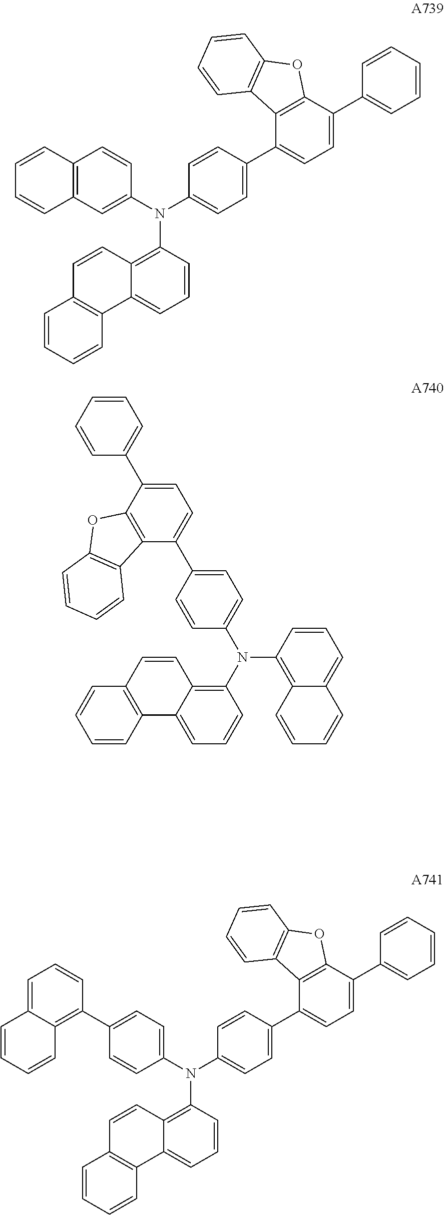 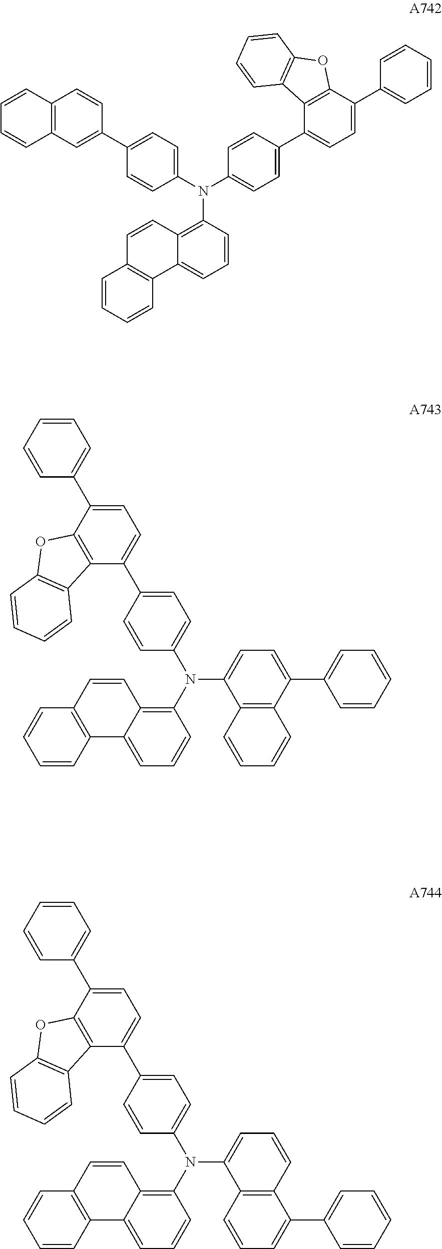 [SMILES: c1ccc(-c2ccc(-c3ccc(N(c4ccc(-c5ccc6ccccc6c5)cc4)c4cccc5c4ccc4ccccc45)cc3)c3c2oc2ccccc23)cc1 ; c1ccc(-c2ccc(N(c3ccc(-c4ccc(-c5ccccc5)c5oc6ccccc6c45)cc3)c3cccc4c3ccc3ccccc34)c3ccccc23)cc1 ; c1ccc(-c2cccc3c(N(c4ccc(-c5ccc(-c6ccccc6)c6oc7ccccc7c56)cc4)c4cccc5c4ccc4ccccc45)cccc23)cc1] 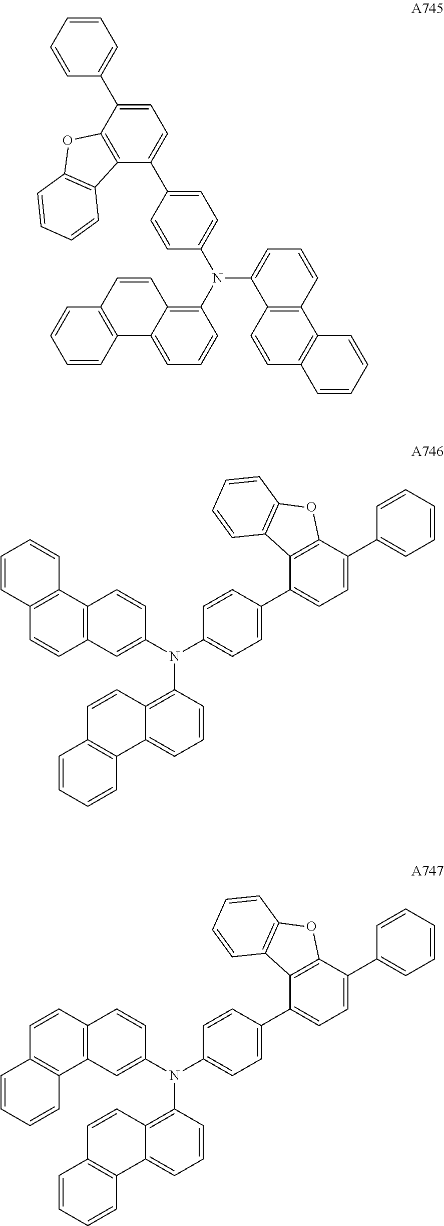 [SMILES: c1ccc(-c2ccc(-c3ccc(N(c4cccc5c4ccc4ccccc45)c4cccc5c4ccc4ccccc45)cc3)c3c2oc2ccccc23)cc1 ; c1ccc(-c2ccc(-c3ccc(N(c4ccc5c(ccc6ccccc65)c4)c4cccc5c4ccc4ccccc45)cc3)c3c2oc2ccccc23)cc1 ; c1ccc(-c2ccc(-c3ccc(N(c4ccc5ccc6ccccc6c5c4)c4cccc5c4ccc4ccccc45)cc3)c3c2oc2ccccc23)cc1] 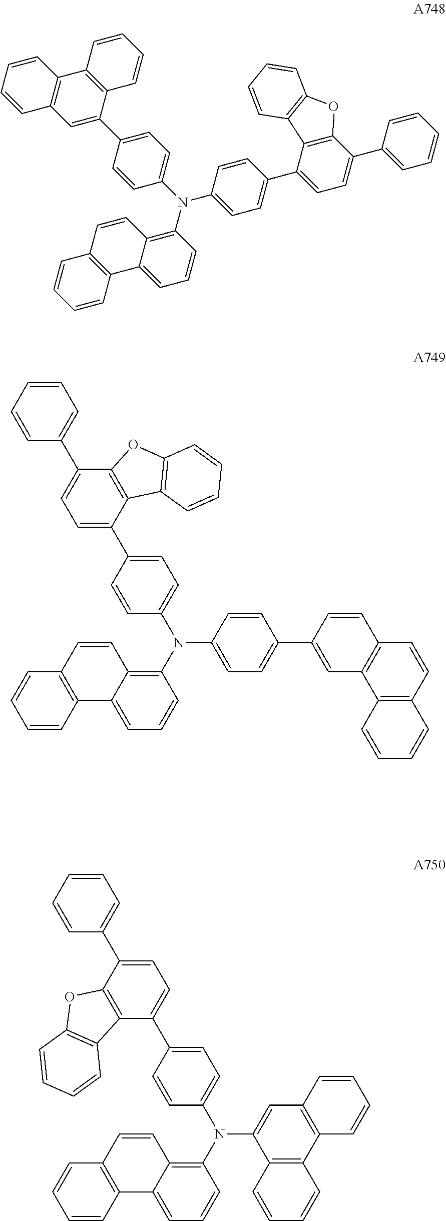 [SMILES: c1ccc(-c2ccc(-c3ccc(N(c4ccc(-c5cc6ccccc6c6ccccc56)cc4)c4cccc5c4ccc4ccccc45)cc3)c3c2oc2ccccc23)cc1 ; c1ccc(-c2ccc(-c3ccc(N(c4ccc(-c5ccc6ccc7ccccc7c6c5)cc4)c4cccc5c4ccc4ccccc45)cc3)c3c2oc2ccccc23)cc1 ; c1ccc(-c2ccc(-c3ccc(N(c4cccc5c4ccc4ccccc45)c4cc5ccccc5c5ccccc45)cc3)c3c2oc2ccccc23)cc1] 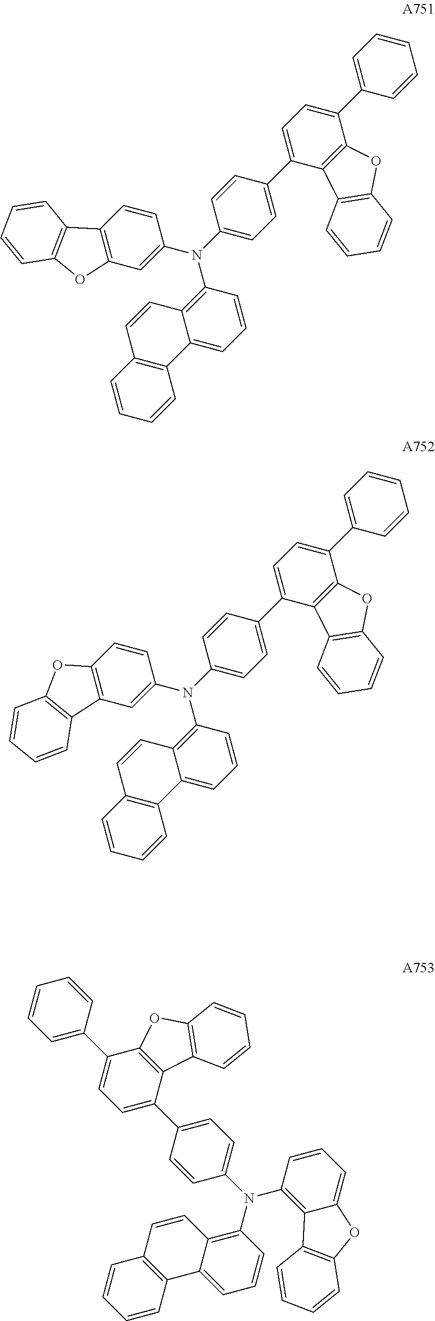 [SMILES: c1ccc(-c2ccc(-c3ccc(N(c4ccc5c(c4)oc4ccccc45)c4cccc5c4ccc4ccccc45)cc3)c3c2oc2ccccc23)cc1 ; c1ccc(-c2ccc(-c3ccc(N(c4ccc5oc6ccccc6c5c4)c4cccc5c4ccc4ccccc45)cc3)c3c2oc2ccccc23)cc1 ; c1ccc(-c2ccc(-c3ccc(N(c4cccc5c4ccc4ccccc45)c4cccc5oc6ccccc6c45)cc3)c3c2oc2ccccc23)cc1] 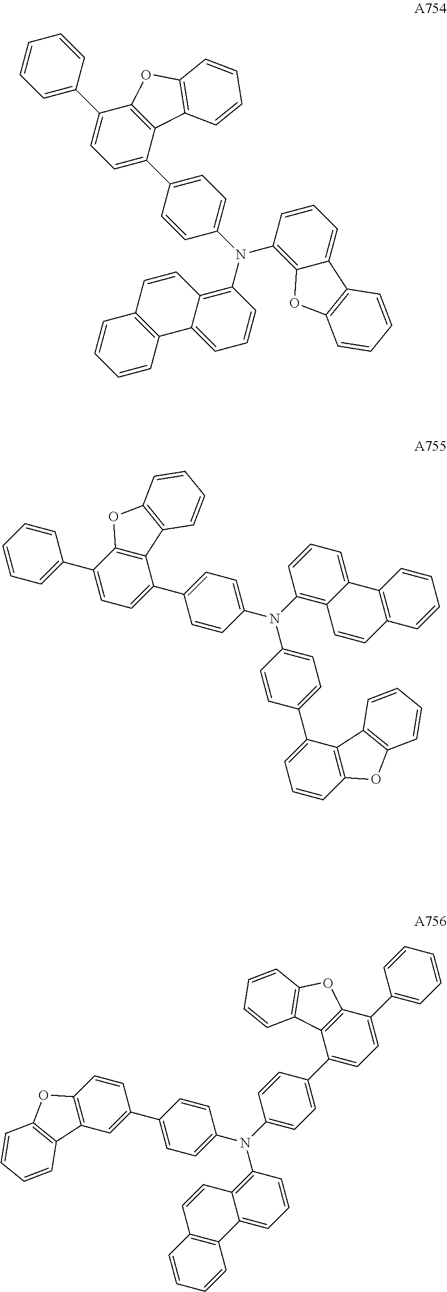 [SMILES: c1ccc(-c2ccc(-c3ccc(N(c4cccc5c4ccc4ccccc45)c4cccc5c4oc4ccccc45)cc3)c3c2oc2ccccc23)cc1 ; c1ccc(-c2ccc(-c3ccc(N(c4ccc(-c5cccc6oc7ccccc7c56)cc4)c4cccc5c4ccc4ccccc45)cc3)c3c2oc2ccccc23)cc1 ; c1ccc(-c2ccc(-c3ccc(N(c4ccc(-c5ccc6oc7ccccc7c6c5)cc4)c4cccc5c4ccc4ccccc45)cc3)c3c2oc2ccccc23)cc1] 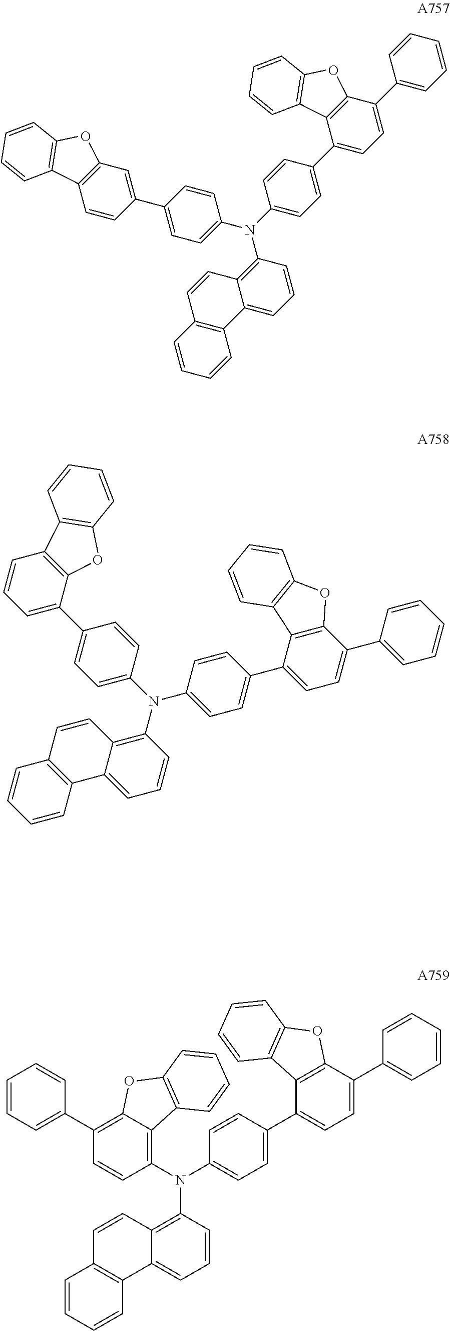 [SMILES: c1ccc(-c2ccc(-c3ccc(N(c4ccc(-c5ccc6c(c5)oc5ccccc56)cc4)c4cccc5c4ccc4ccccc45)cc3)c3c2oc2ccccc23)cc1 ; c1ccc(-c2ccc(-c3ccc(N(c4ccc(-c5cccc6c5oc5ccccc56)cc4)c4cccc5c4ccc4ccccc45)cc3)c3c2oc2ccccc23)cc1 ; c1ccc(-c2ccc(-c3ccc(N(c4cccc5c4ccc4ccccc45)c4ccc(-c5ccccc5)c5oc6ccccc6c45)cc3)c3c2oc2ccccc23)cc1] 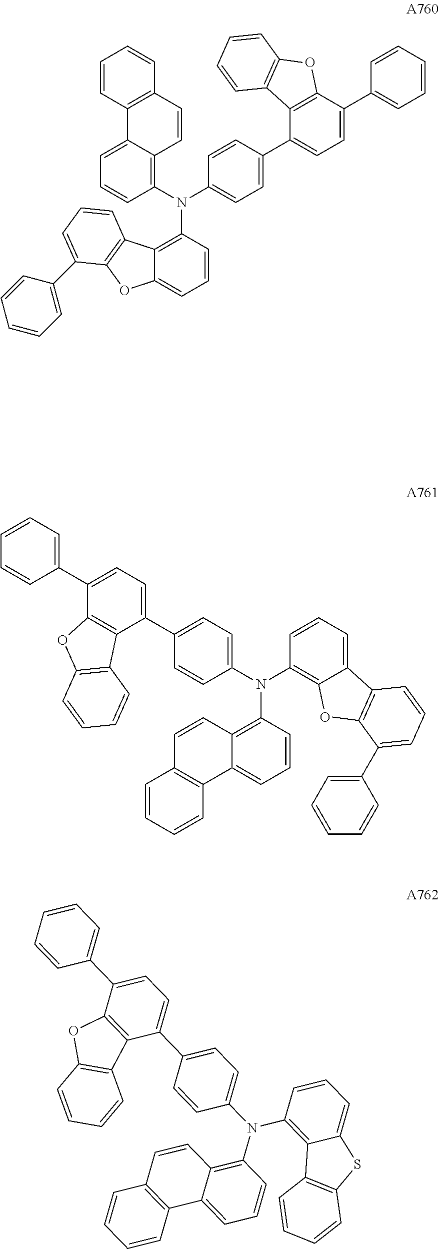 [SMILES: c1ccc(-c2cccc3c2oc2cccc(N(c4ccc(-c5ccc(-c6ccccc6)c6oc7ccccc7c56)cc4)c4cccc5c4ccc4ccccc45)c23)cc1 ; c1ccc(-c2cccc3c2oc2c(N(c4ccc(-c5ccc(-c6ccccc6)c6oc7ccccc7c56)cc4)c4cccc5c4ccc4ccccc45)cccc23)cc1 ; c1ccc(-c2ccc(-c3ccc(N(c4cccc5c4ccc4ccccc45)c4cccc5sc6ccccc6c45)cc3)c3c2oc2ccccc23)cc1] 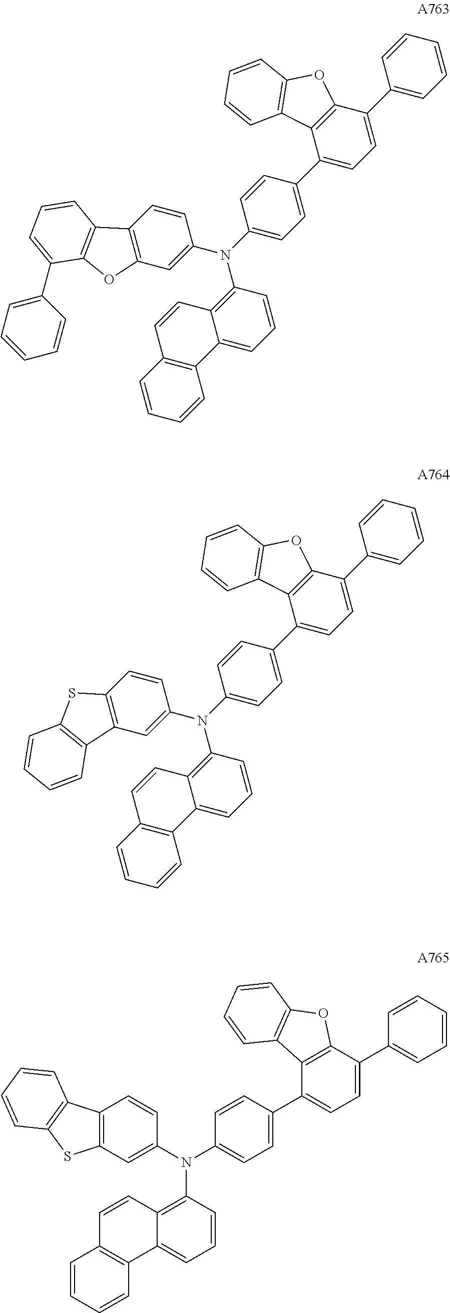 [SMILES: c1ccc(-c2cccc3c2oc2cc(N(c4ccc(-c5ccc(-c6ccccc6)c6oc7ccccc7c56)cc4)c4cccc5c4ccc4ccccc45)ccc23)cc1 ; c1ccc(-c2ccc(-c3ccc(N(c4ccc5sc6ccccc6c5c4)c4cccc5c4ccc4ccccc45)cc3)c3c2oc2ccccc23)cc1 ; c1ccc(-c2ccc(-c3ccc(N(c4ccc5c(c4)sc4ccccc45)c4cccc5c4ccc4ccccc45)cc3)c3c2oc2ccccc23)cc1] 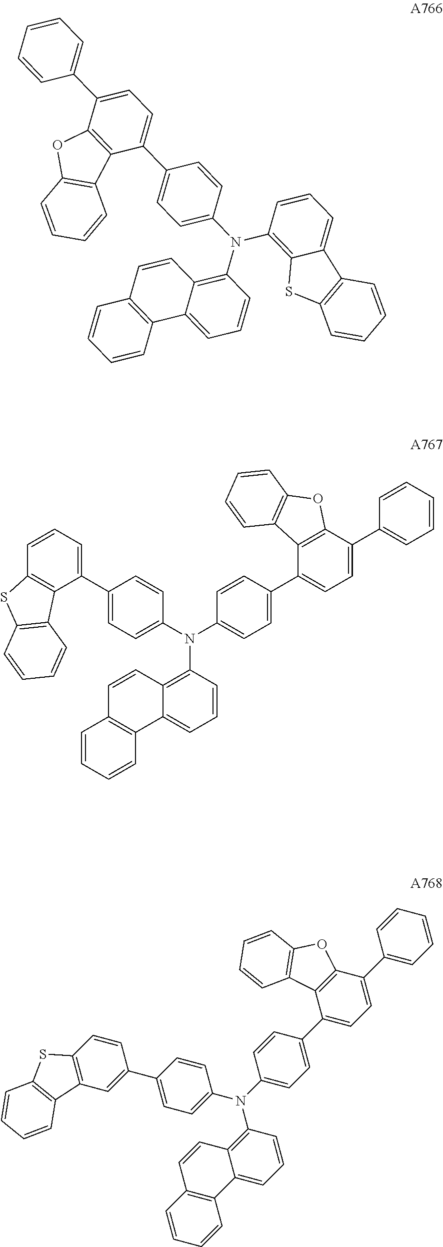 [SMILES: c1ccc(-c2ccc(-c3ccc(N(c4cccc5c4ccc4ccccc45)c4cccc5c4sc4ccccc45)cc3)c3c2oc2ccccc23)cc1 ; c1ccc(-c2ccc(-c3ccc(N(c4ccc(-c5cccc6sc7ccccc7c56)cc4)c4cccc5c4ccc4ccccc45)cc3)c3c2oc2ccccc23)cc1 ; c1ccc(-c2ccc(-c3ccc(N(c4ccc(-c5ccc6sc7ccccc7c6c5)cc4)c4cccc5c4ccc4ccccc45)cc3)c3c2oc2ccccc23)cc1] 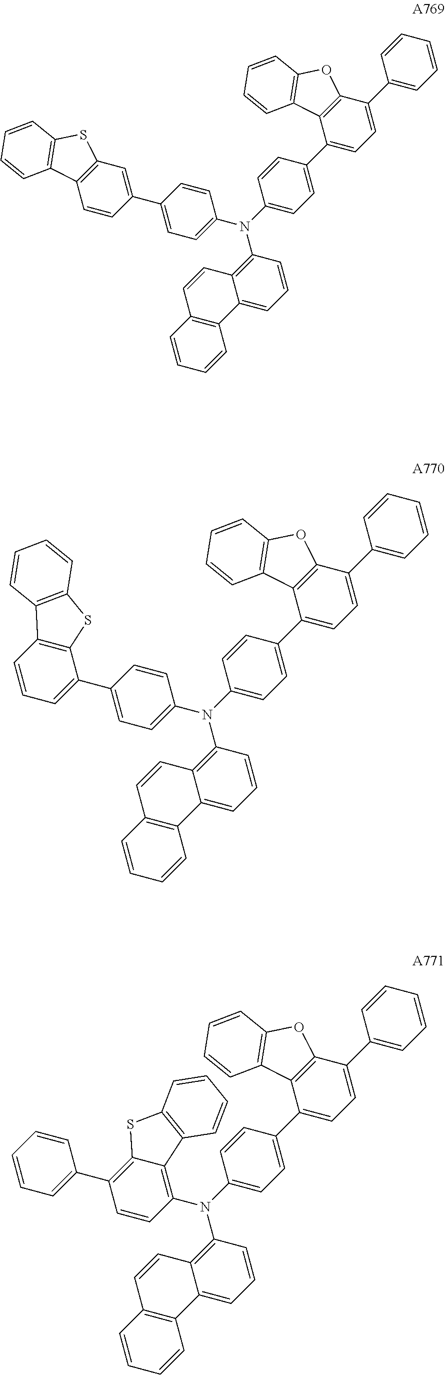 [SMILES: c1ccc(-c2ccc(-c3ccc(N(c4ccc(-c5ccc6c(c5)sc5ccccc56)cc4)c4cccc5c4ccc4ccccc45)cc3)c3c2oc2ccccc23)cc1 ; c1ccc(-c2ccc(-c3ccc(N(c4ccc(-c5cccc6c5sc5ccccc56)cc4)c4cccc5c4ccc4ccccc45)cc3)c3c2oc2ccccc23)cc1 ; c1ccc(-c2ccc(-c3ccc(N(c4cccc5c4ccc4ccccc45)c4ccc(-c5ccccc5)c5sc6ccccc6c45)cc3)c3c2oc2ccccc23)cc1] 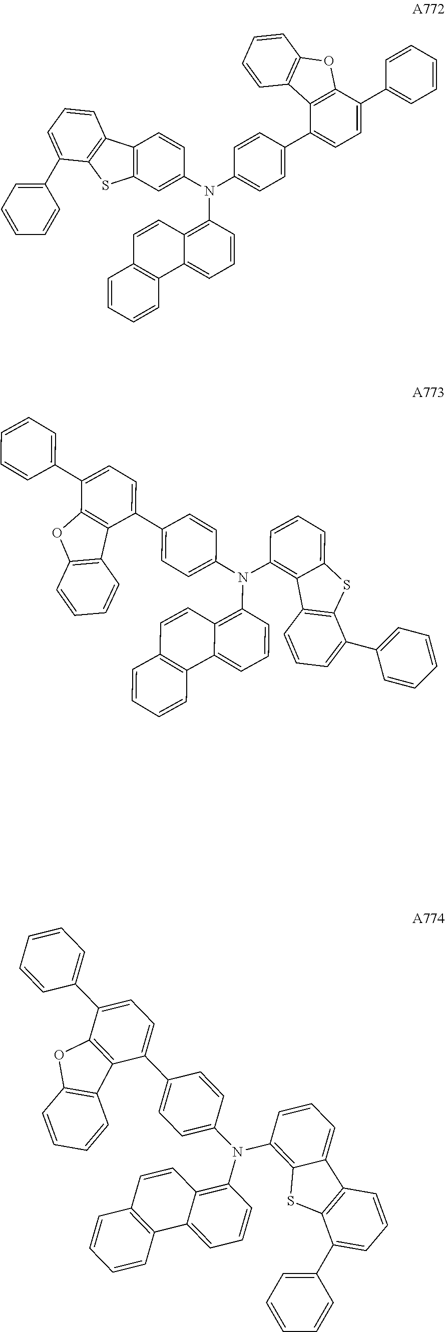 [SMILES: c1ccc(-c2ccc(-c3ccc(N(c4ccc5c(c4)sc4c(-c6ccccc6)cccc45)c4cccc5c4ccc4ccccc45)cc3)c3c2oc2ccccc23)cc1 ; c1ccc(-c2ccc(-c3ccc(N(c4cccc5c4ccc4ccccc45)c4cccc5sc6c(-c7ccccc7)cccc6c45)cc3)c3c2oc2ccccc23)cc1 ; c1ccc(-c2ccc(-c3ccc(N(c4cccc5c4ccc4ccccc45)c4cccc5c4sc4c(-c6ccccc6)cccc45)cc3)c3c2oc2ccccc23)cc1] 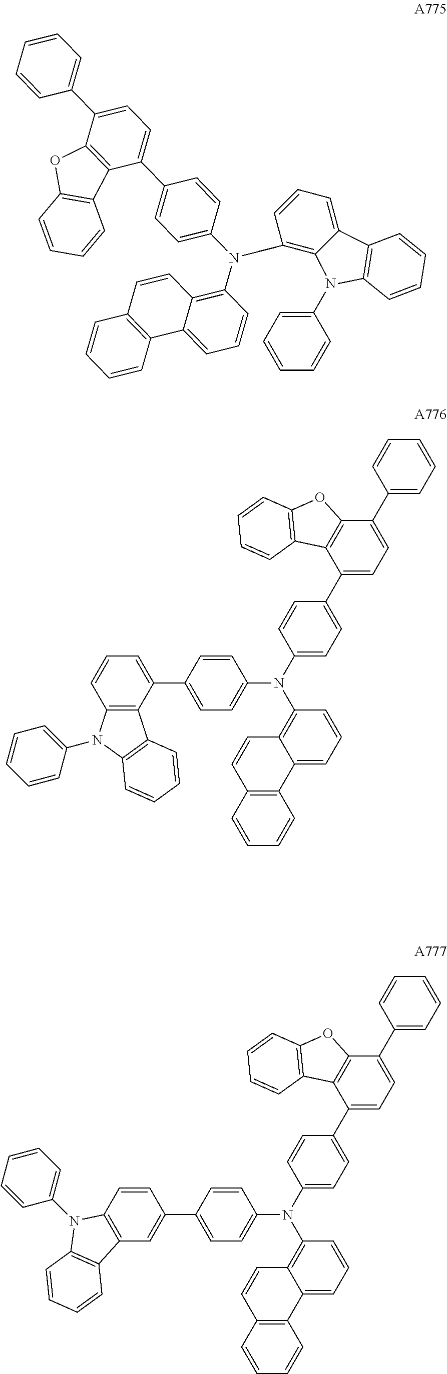 [SMILES: c1ccc(-c2ccc(-c3ccc(N(c4cccc5c4ccc4ccccc45)c4cccc5c6ccccc6n(-c6ccccc6)c45)cc3)c3c2oc2ccccc23)cc1 ; c1ccc(-c2ccc(-c3ccc(N(c4ccc(-c5cccc6c5c5ccccc5n6-c5ccccc5)cc4)c4cccc5c4ccc4ccccc45)cc3)c3c2oc2ccccc23)cc1 ; c1ccc(-c2ccc(-c3ccc(N(c4ccc(-c5ccc6c(c5)c5ccccc5n6-c5ccccc5)cc4)c4cccc5c4ccc4ccccc45)cc3)c3c2oc2ccccc23)cc1] 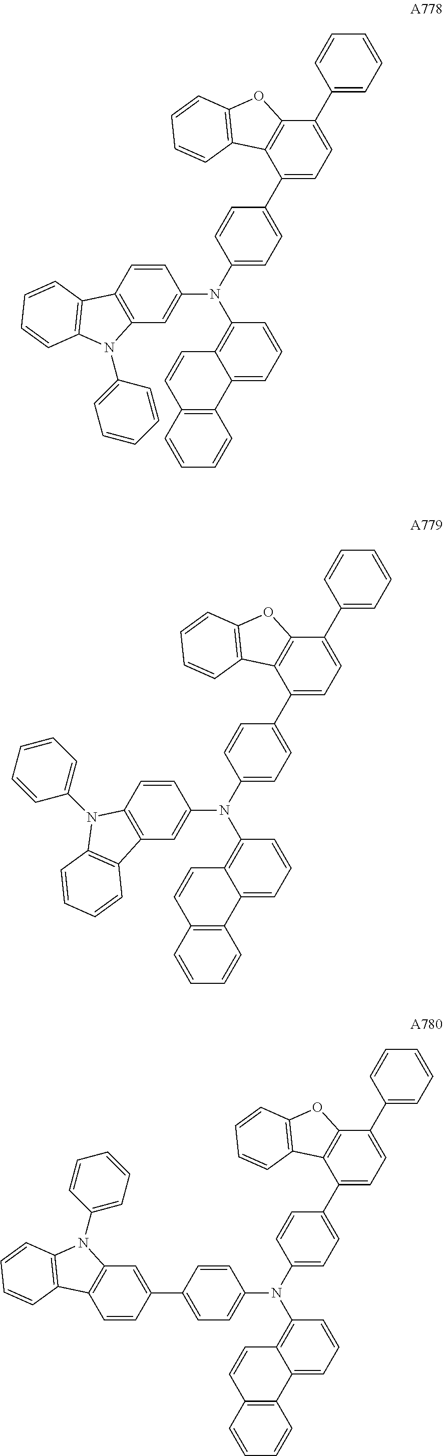 [SMILES: c1ccc(-c2ccc(-c3ccc(N(c4ccc5c6ccccc6n(-c6ccccc6)c5c4)c4cccc5c4ccc4ccccc45)cc3)c3c2oc2ccccc23)cc1 ; c1ccc(-c2ccc(-c3ccc(N(c4ccc5c(c4)c4ccccc4n5-c4ccccc4)c4cccc5c4ccc4ccccc45)cc3)c3c2oc2ccccc23)cc1 ; c1ccc(-c2ccc(-c3ccc(N(c4ccc(-c5ccc6c7ccccc7n(-c7ccccc7)c6c5)cc4)c4cccc5c4ccc4ccccc45)cc3)c3c2oc2ccccc23)cc1] 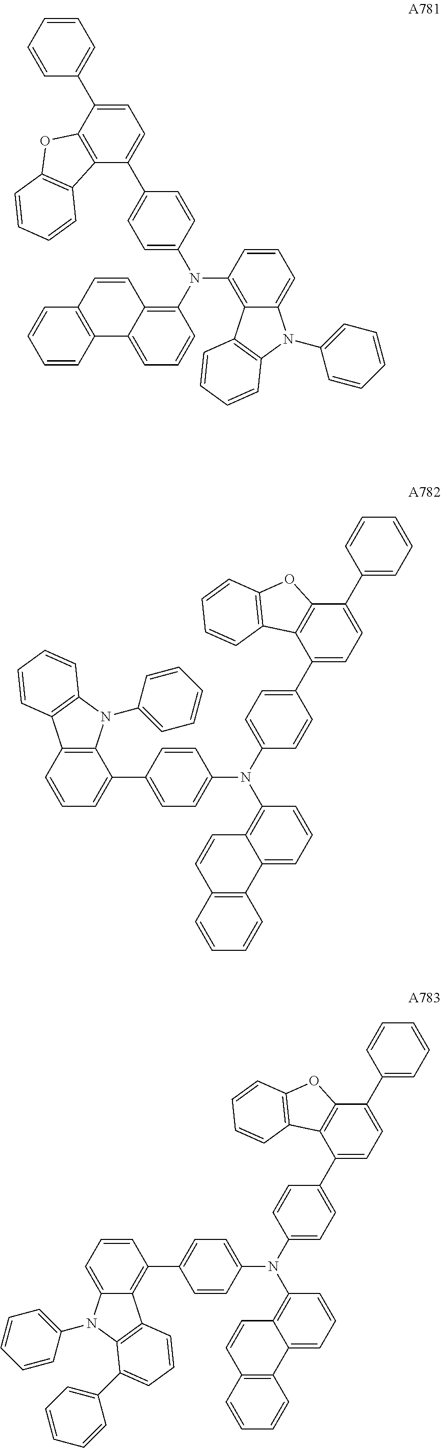 [SMILES: c1ccc(-c2ccc(-c3ccc(N(c4cccc5c4ccc4ccccc45)c4cccc5c4c4ccccc4n5-c4ccccc4)cc3)c3c2oc2ccccc23)cc1 ; c1ccc(-c2ccc(-c3ccc(N(c4ccc(-c5cccc6c7ccccc7n(-c7ccccc7)c56)cc4)c4cccc5c4ccc4ccccc45)cc3)c3c2oc2ccccc23)cc1 ; c1ccc(-c2ccc(-c3ccc(N(c4ccc(-c5cccc6c5c5cccc(-c7ccccc7)c5n6-c5ccccc5)cc4)c4cccc5c4ccc4ccccc45)cc3)c3c2oc2ccccc23)cc1] 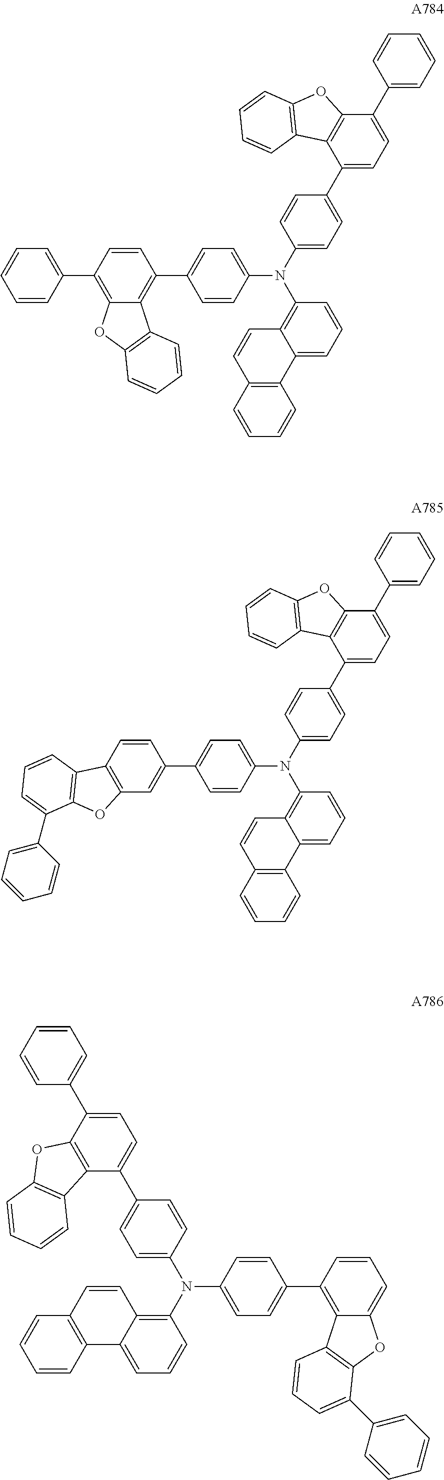 [SMILES: c1ccc(-c2ccc(-c3ccc(N(c4ccc(-c5ccc(-c6ccccc6)c6oc7ccccc7c56)cc4)c4cccc5c4ccc4ccccc45)cc3)c3c2oc2ccccc23)cc1 ; c1ccc(-c2cccc3c2oc2cc(-c4ccc(N(c5ccc(-c6ccc(-c7ccccc7)c7oc8ccccc8c67)cc5)c5cccc6c5ccc5ccccc56)cc4)ccc23)cc1 ; c1ccc(-c2cccc3c2oc2cccc(-c4ccc(N(c5ccc(-c6ccc(-c7ccccc7)c7oc8ccccc8c67)cc5)c5cccc6c5ccc5ccccc56)cc4)c23)cc1] 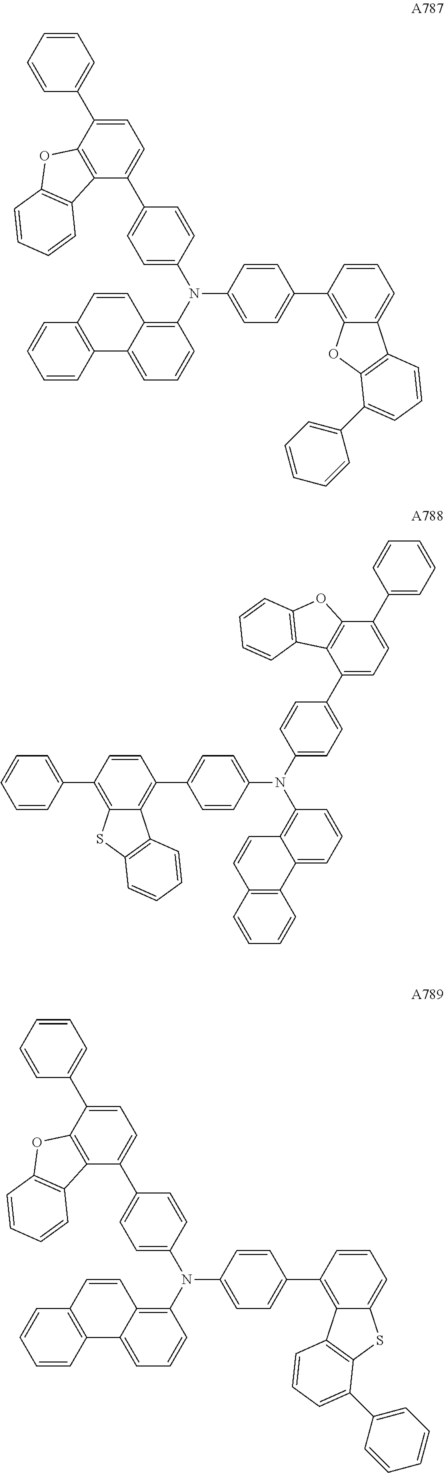 [SMILES: c1ccc(-c2cccc3c2oc2c(-c4ccc(N(c5ccc(-c6ccc(-c7ccccc7)c7oc8ccccc8c67)cc5)c5cccc6c5ccc5ccccc56)cc4)cccc23)cc1 ; c1ccc(-c2ccc(-c3ccc(N(c4ccc(-c5ccc(-c6ccccc6)c6sc7ccccc7c56)cc4)c4cccc5c4ccc4ccccc45)cc3)c3c2oc2ccccc23)cc1 ; c1ccc(-c2ccc(-c3ccc(N(c4ccc(-c5cccc6sc7c(-c8ccccc8)cccc7c56)cc4)c4cccc5c4ccc4ccccc45)cc3)c3c2oc2ccccc23)cc1] 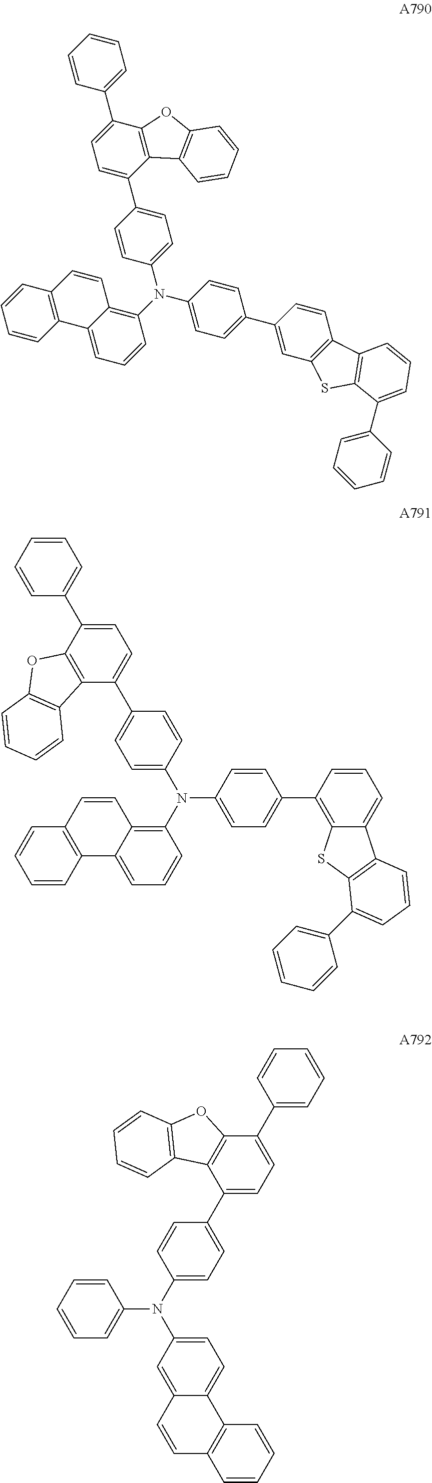 [SMILES: c1ccc(-c2ccc(-c3ccc(N(c4ccc(-c5ccc6c(c5)sc5c(-c7ccccc7)cccc56)cc4)c4cccc5c4ccc4ccccc45)cc3)c3c2oc2ccccc23)cc1 ; c1ccc(-c2ccc(-c3ccc(N(c4ccc(-c5cccc6c5sc5c(-c7ccccc7)cccc56)cc4)c4cccc5c4ccc4ccccc45)cc3)c3c2oc2ccccc23)cc1 ; c1ccc(-c2ccc(-c3ccc(N(c4ccccc4)c4ccc5c(ccc6ccccc65)c4)cc3)c3c2oc2ccccc23)cc1] 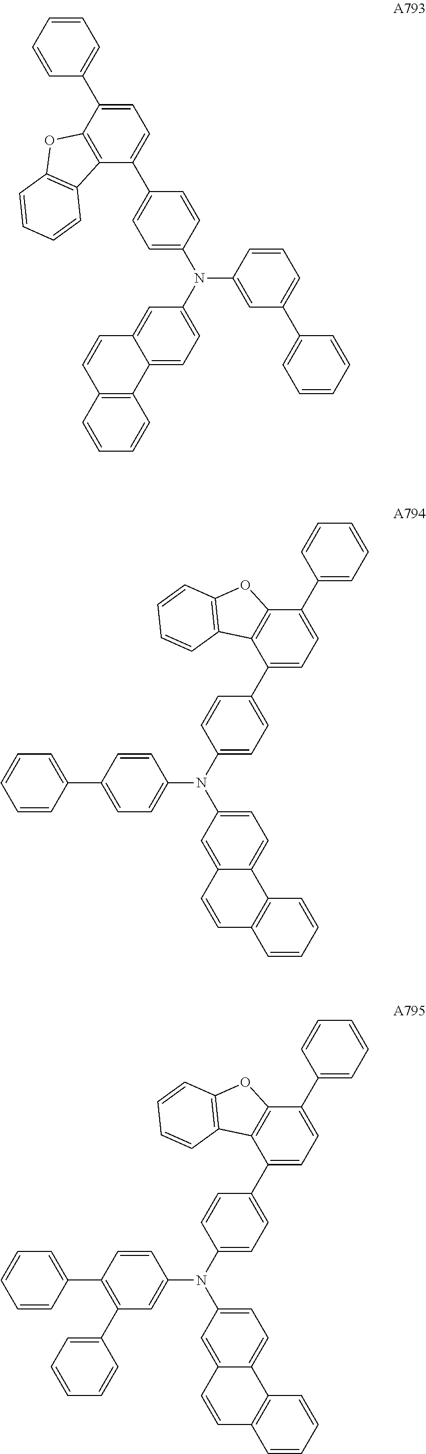 [SMILES: c1ccc(-c2cccc(N(c3ccc(-c4ccc(-c5ccccc5)c5oc6ccccc6c45)cc3)c3ccc4c(ccc5ccccc54)c3)c2)cc1 ; c1ccc(-c2ccc(N(c3ccc(-c4ccc(-c5ccccc5)c5oc6ccccc6c45)cc3)c3ccc4c(ccc5ccccc54)c3)cc2)cc1 ; c1ccc(-c2ccc(N(c3ccc(-c4ccc(-c5ccccc5)c5oc6ccccc6c45)cc3)c3ccc4c(ccc5ccccc54)c3)cc2-c2ccccc2)cc1] 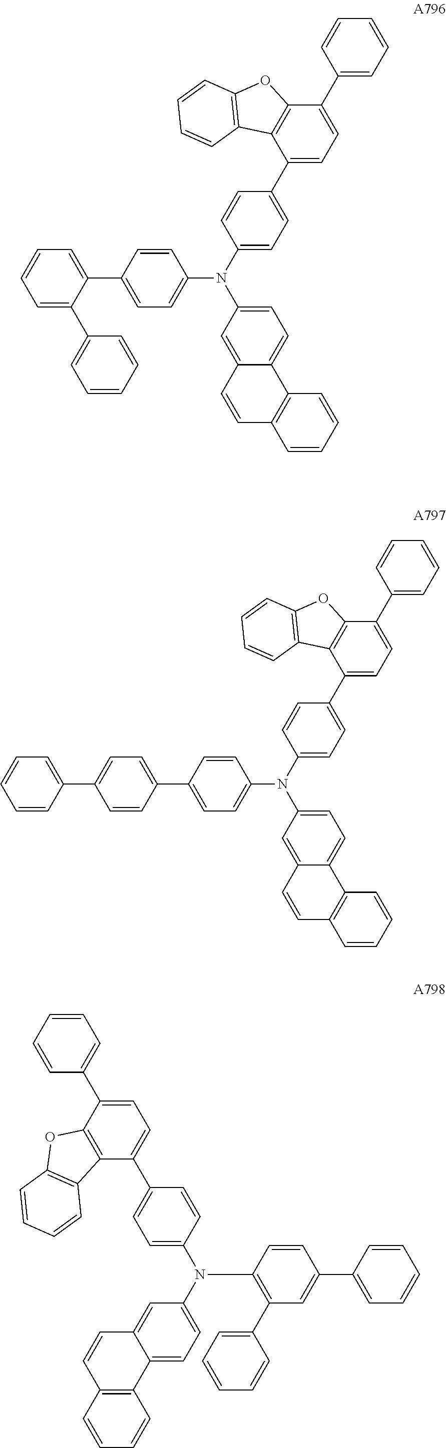 [SMILES: c1ccc(-c2ccccc2-c2ccc(N(c3ccc(-c4ccc(-c5ccccc5)c5oc6ccccc6c45)cc3)c3ccc4c(ccc5ccccc54)c3)cc2)cc1 ; c1ccc(-c2ccc(-c3ccc(N(c4ccc(-c5ccc(-c6ccccc6)c6oc7ccccc7c56)cc4)c4ccc5c(ccc6ccccc65)c4)cc3)cc2)cc1 ; c1ccc(-c2ccc(N(c3ccc(-c4ccc(-c5ccccc5)c5oc6ccccc6c45)cc3)c3ccc4c(ccc5ccccc54)c3)c(-c3ccccc3)c2)cc1] 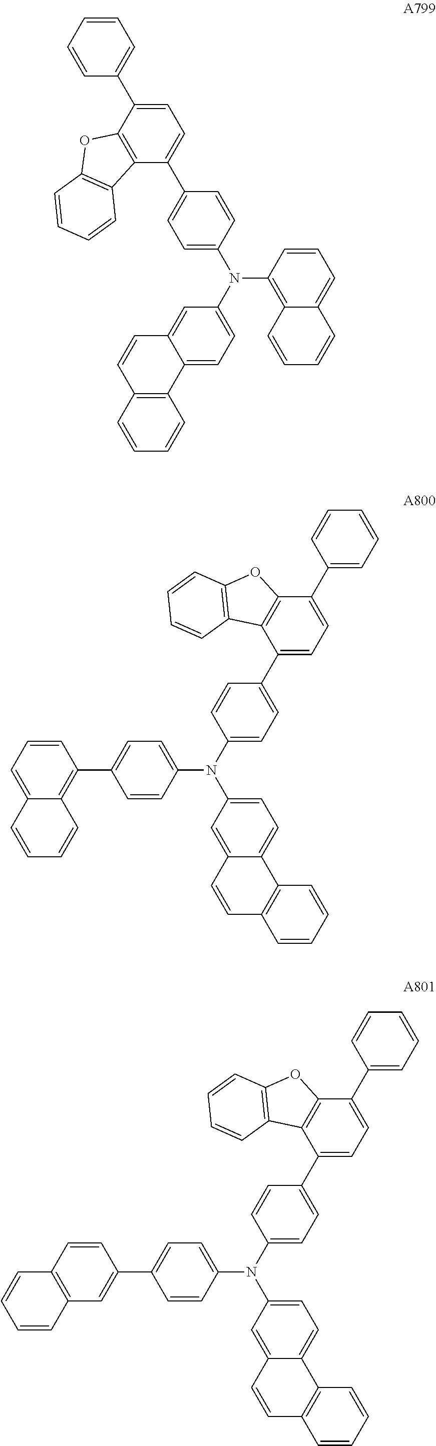 [SMILES: c1ccc(-c2ccc(-c3ccc(N(c4ccc5c(ccc6ccccc65)c4)c4cccc5ccccc45)cc3)c3c2oc2ccccc23)cc1 ; c1ccc(-c2ccc(-c3ccc(N(c4ccc(-c5cccc6ccccc56)cc4)c4ccc5c(ccc6ccccc65)c4)cc3)c3c2oc2ccccc23)cc1 ; c1ccc(-c2ccc(-c3ccc(N(c4ccc(-c5ccc6ccccc6c5)cc4)c4ccc5c(ccc6ccccc65)c4)cc3)c3c2oc2ccccc23)cc1] 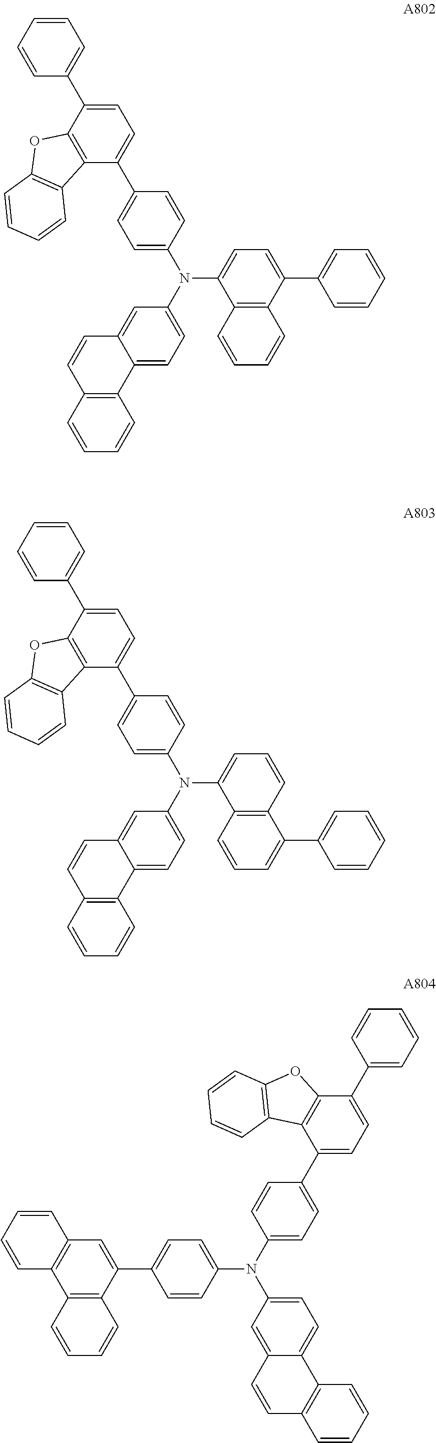 [SMILES: c1ccc(-c2ccc(N(c3ccc(-c4ccc(-c5ccccc5)c5oc6ccccc6c45)cc3)c3ccc4c(ccc5ccccc54)c3)c3ccccc23)cc1 ; c1ccc(-c2cccc3c(N(c4ccc(-c5ccc(-c6ccccc6)c6oc7ccccc7c56)cc4)c4ccc5c(ccc6ccccc65)c4)cccc23)cc1 ; c1ccc(-c2ccc(-c3ccc(N(c4ccc(-c5cc6ccccc6c6ccccc56)cc4)c4ccc5c(ccc6ccccc65)c4)cc3)c3c2oc2ccccc23)cc1] 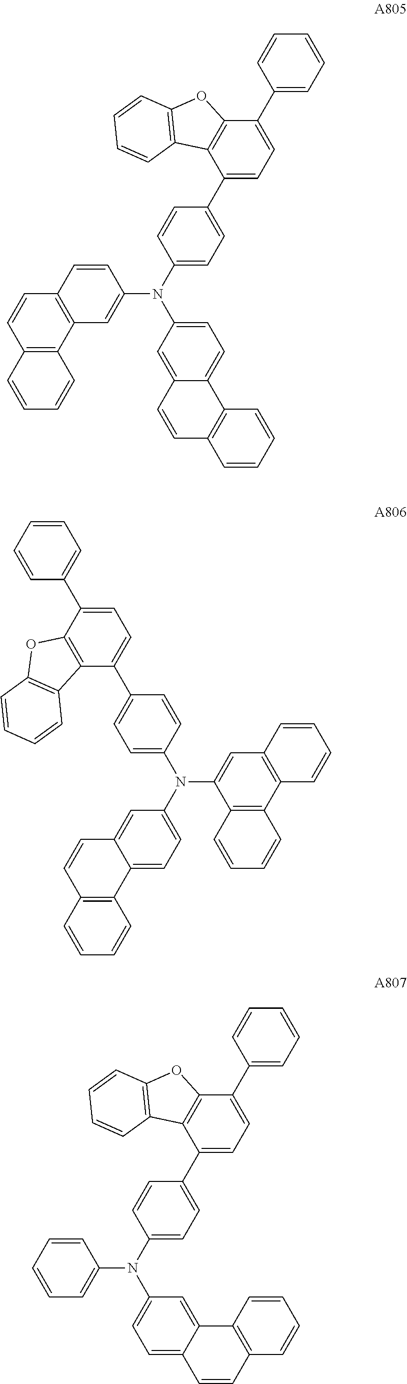 [SMILES: c1ccc(-c2ccc(-c3ccc(N(c4ccc5c(ccc6ccccc65)c4)c4ccc5ccc6ccccc6c5c4)cc3)c3c2oc2ccccc23)cc1 ; c1ccc(-c2ccc(-c3ccc(N(c4ccc5c(ccc6ccccc65)c4)c4cc5ccccc5c5ccccc45)cc3)c3c2oc2ccccc23)cc1 ; c1ccc(-c2ccc(-c3ccc(N(c4ccccc4)c4ccc5ccc6ccccc6c5c4)cc3)c3c2oc2ccccc23)cc1] 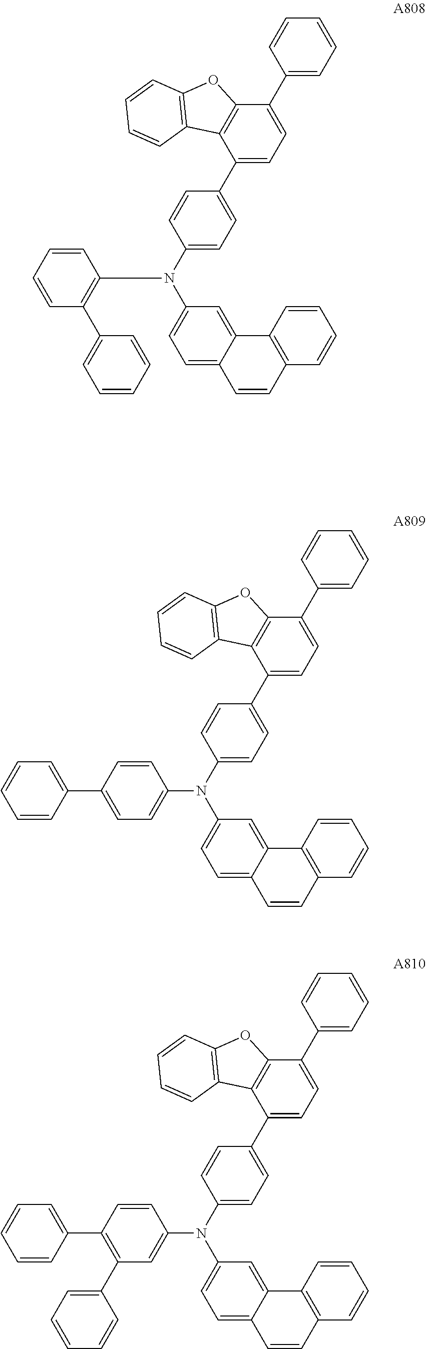 [SMILES: c1ccc(-c2ccccc2N(c2ccc(-c3ccc(-c4ccccc4)c4oc5ccccc5c34)cc2)c2ccc3ccc4ccccc4c3c2)cc1 ; c1ccc(-c2ccc(N(c3ccc(-c4ccc(-c5ccccc5)c5oc6ccccc6c45)cc3)c3ccc4ccc5ccccc5c4c3)cc2)cc1 ; c1ccc(-c2ccc(N(c3ccc(-c4ccc(-c5ccccc5)c5oc6ccccc6c45)cc3)c3ccc4ccc5ccccc5c4c3)cc2-c2ccccc2)cc1] 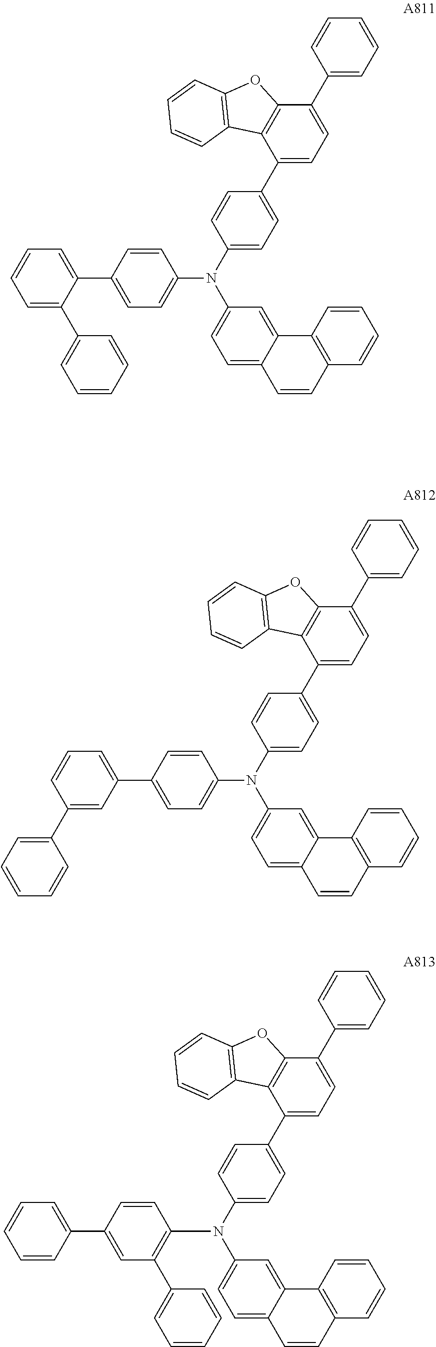 [SMILES: c1ccc(-c2ccccc2-c2ccc(N(c3ccc(-c4ccc(-c5ccccc5)c5oc6ccccc6c45)cc3)c3ccc4ccc5ccccc5c4c3)cc2)cc1 ; c1ccc(-c2cccc(-c3ccc(N(c4ccc(-c5ccc(-c6ccccc6)c6oc7ccccc7c56)cc4)c4ccc5ccc6ccccc6c5c4)cc3)c2)cc1 ; c1ccc(-c2ccc(N(c3ccc(-c4ccc(-c5ccccc5)c5oc6ccccc6c45)cc3)c3ccc4ccc5ccccc5c4c3)c(-c3ccccc3)c2)cc1] 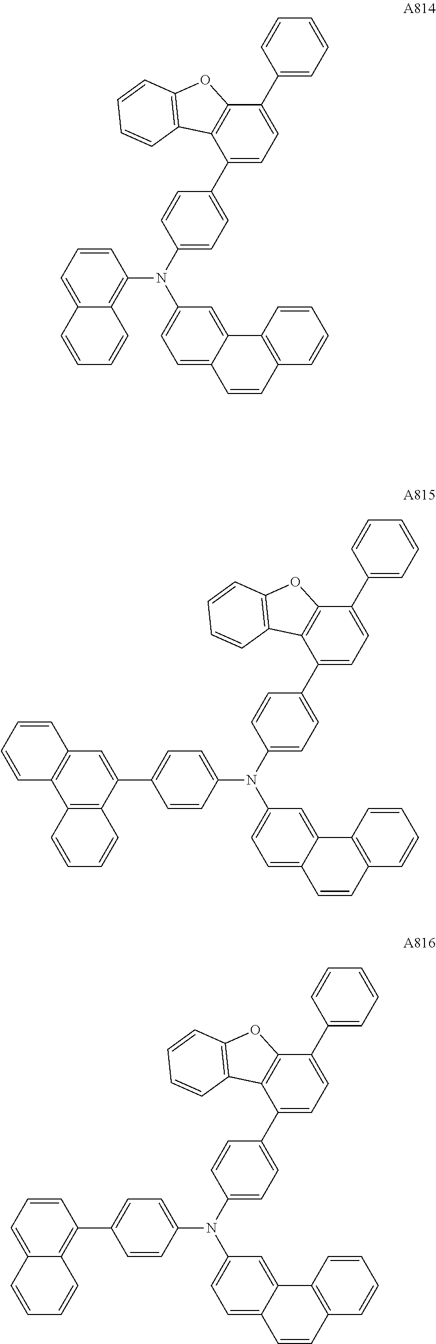 [SMILES: c1ccc(-c2ccc(-c3ccc(N(c4ccc5ccc6ccccc6c5c4)c4cccc5ccccc45)cc3)c3c2oc2ccccc23)cc1 ; c1ccc(-c2ccc(-c3ccc(N(c4ccc(-c5cc6ccccc6c6ccccc56)cc4)c4ccc5ccc6ccccc6c5c4)cc3)c3c2oc2ccccc23)cc1 ; c1ccc(-c2ccc(-c3ccc(N(c4ccc(-c5cccc6ccccc56)cc4)c4ccc5ccc6ccccc6c5c4)cc3)c3c2oc2ccccc23)cc1] 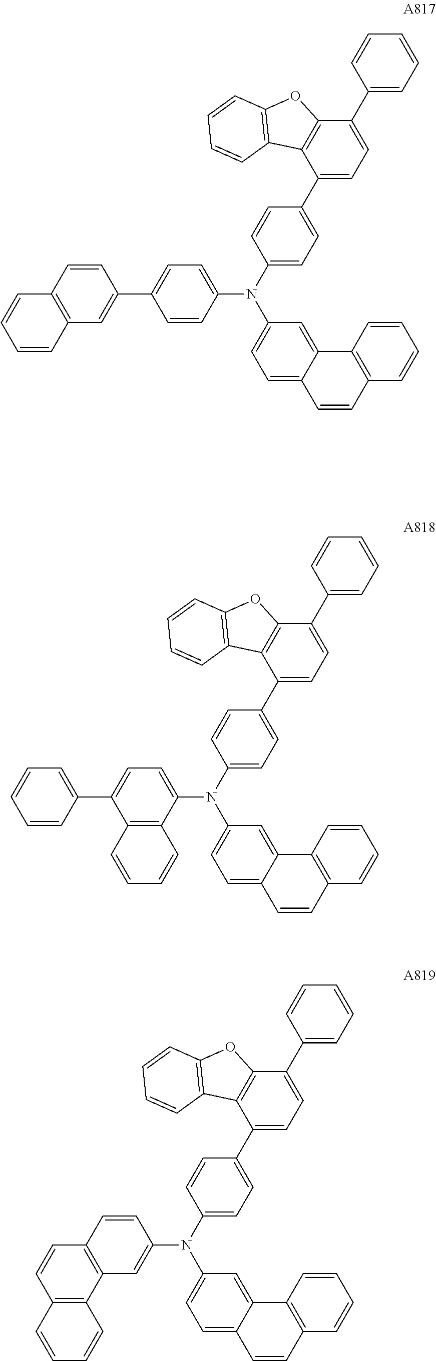 [SMILES: c1ccc(-c2ccc(-c3ccc(N(c4ccc(-c5ccc6ccccc6c5)cc4)c4ccc5ccc6ccccc6c5c4)cc3)c3c2oc2ccccc23)cc1 ; c1ccc(-c2ccc(N(c3ccc(-c4ccc(-c5ccccc5)c5oc6ccccc6c45)cc3)c3ccc4ccc5ccccc5c4c3)c3ccccc23)cc1 ; c1ccc(-c2ccc(-c3ccc(N(c4ccc5ccc6ccccc6c5c4)c4ccc5ccc6ccccc6c5c4)cc3)c3c2oc2ccccc23)cc1] 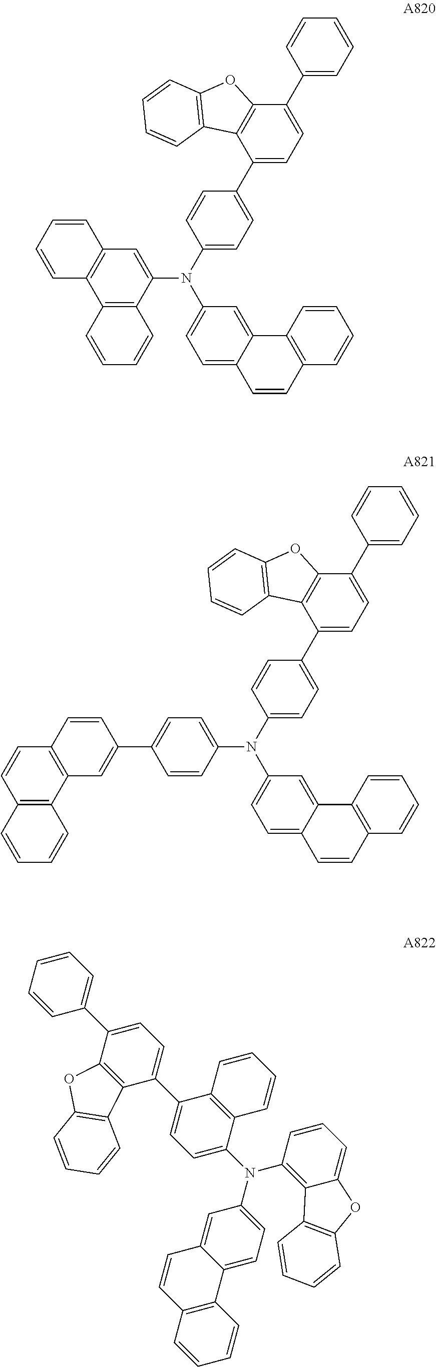 [SMILES: c1ccc(-c2ccc(-c3ccc(N(c4ccc5ccc6ccccc6c5c4)c4cc5ccccc5c5ccccc45)cc3)c3c2oc2ccccc23)cc1 ; c1ccc(-c2ccc(-c3ccc(N(c4ccc(-c5ccc6ccc7ccccc7c6c5)cc4)c4ccc5ccc6ccccc6c5c4)cc3)c3c2oc2ccccc23)cc1 ; c1ccc(-c2ccc(-c3ccc(N(c4ccc5c(ccc6ccccc65)c4)c4cccc5oc6ccccc6c45)c4ccccc34)c3c2oc2ccccc23)cc1] 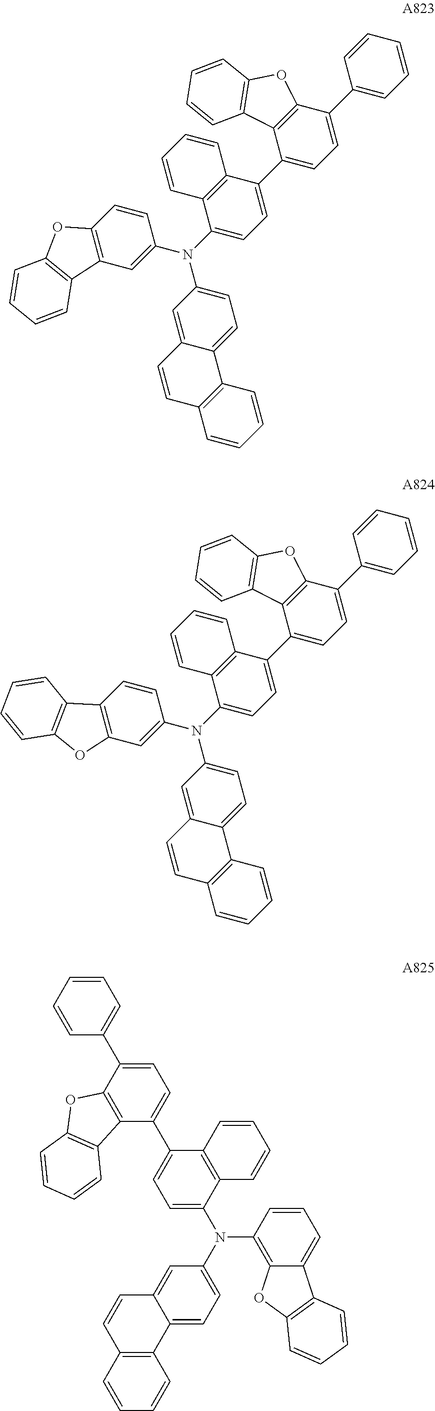 [SMILES: c1ccc(-c2ccc(-c3ccc(N(c4ccc5c(ccc6ccccc65)c4)c4ccc5oc6ccccc6c5c4)c4ccccc34)c3c2oc2ccccc23)cc1 ; c1ccc(-c2ccc(-c3ccc(N(c4ccc5c(ccc6ccccc65)c4)c4ccc5c(c4)oc4ccccc45)c4ccccc34)c3c2oc2ccccc23)cc1 ; c1ccc(-c2ccc(-c3ccc(N(c4ccc5c(ccc6ccccc65)c4)c4cccc5c4oc4ccccc45)c4ccccc34)c3c2oc2ccccc23)cc1] 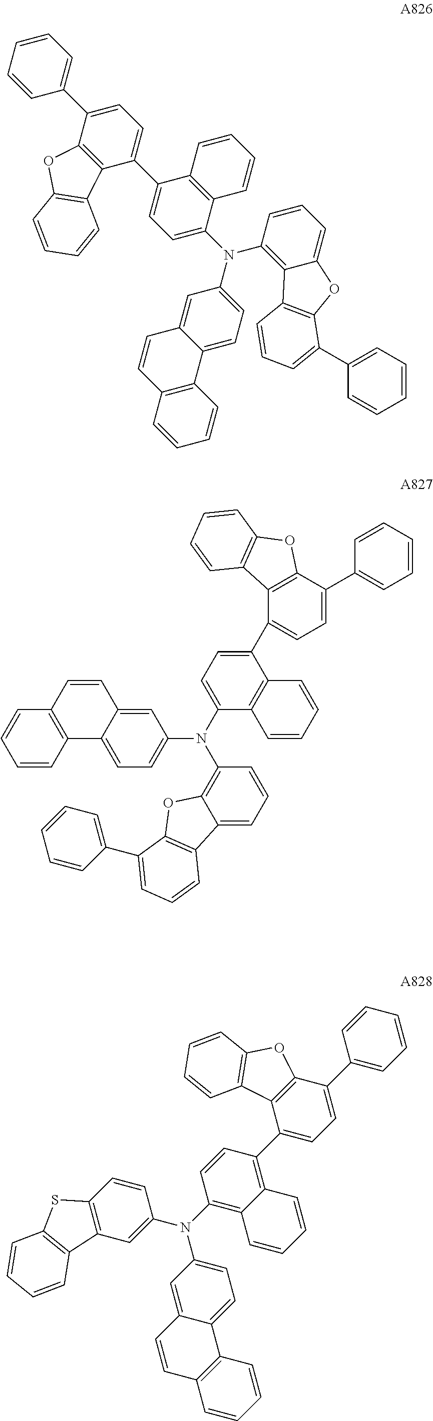 [SMILES: c1ccc(-c2cccc3c2oc2cccc(N(c4ccc5c(ccc6ccccc65)c4)c4ccc(-c5ccc(-c6ccccc6)c6oc7ccccc7c56)c5ccccc45)c23)cc1 ; c1ccc(-c2cccc3c2oc2c(N(c4ccc5c(ccc6ccccc65)c4)c4ccc(-c5ccc(-c6ccccc6)c6oc7ccccc7c56)c5ccccc45)cccc23)cc1 ; c1ccc(-c2ccc(-c3ccc(N(c4ccc5c(ccc6ccccc65)c4)c4ccc5sc6ccccc6c5c4)c4ccccc34)c3c2oc2ccccc23)cc1] 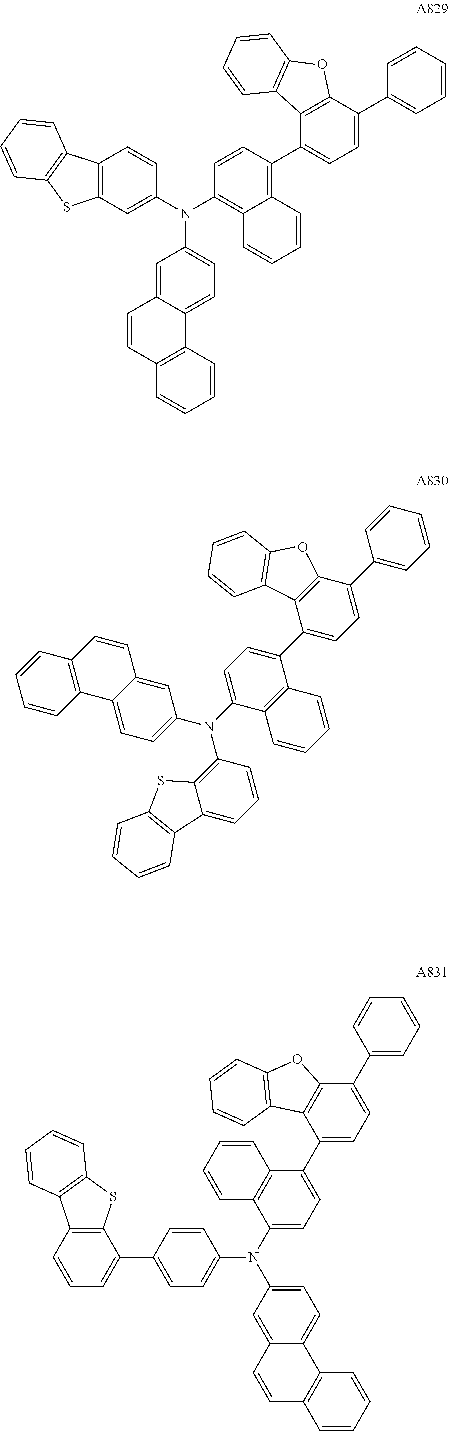 [SMILES: c1ccc(-c2ccc(-c3ccc(N(c4ccc5c(ccc6ccccc65)c4)c4ccc5c(c4)sc4ccccc45)c4ccccc34)c3c2oc2ccccc23)cc1 ; c1ccc(-c2ccc(-c3ccc(N(c4ccc5c(ccc6ccccc65)c4)c4cccc5c4sc4ccccc45)c4ccccc34)c3c2oc2ccccc23)cc1 ; c1ccc(-c2ccc(-c3ccc(N(c4ccc(-c5cccc6c5sc5ccccc56)cc4)c4ccc5c(ccc6ccccc65)c4)c4ccccc34)c3c2oc2ccccc23)cc1] 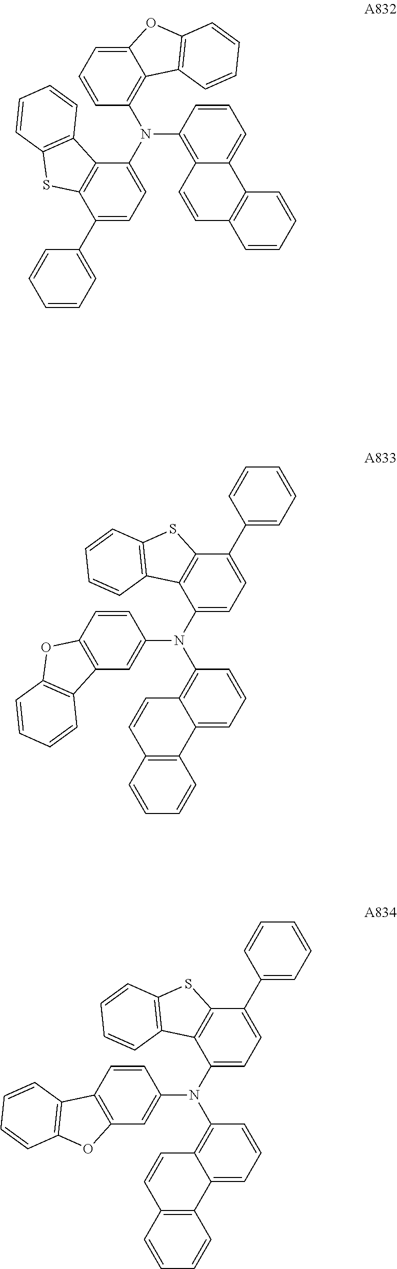 [SMILES: c1ccc(-c2ccc(N(c3cccc4c3ccc3ccccc34)c3cccc4oc5ccccc5c34)c3c2sc2ccccc23)cc1 ; c1ccc(-c2ccc(N(c3ccc4oc5ccccc5c4c3)c3cccc4c3ccc3ccccc34)c3c2sc2ccccc23)cc1 ; c1ccc(-c2ccc(N(c3ccc4c(c3)oc3ccccc34)c3cccc4c3ccc3ccccc34)c3c2sc2ccccc23)cc1] 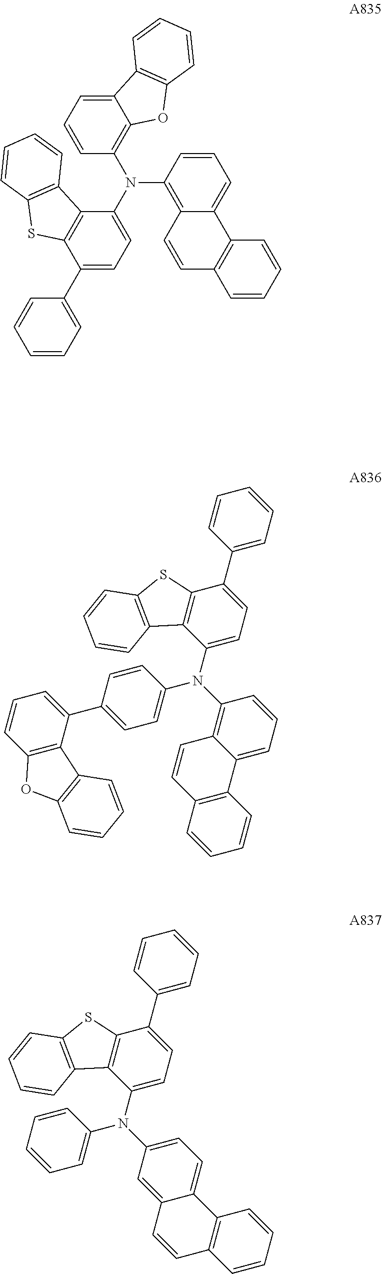 [SMILES: c1ccc(-c2ccc(N(c3cccc4c3ccc3ccccc34)c3cccc4c3oc3ccccc34)c3c2sc2ccccc23)cc1 ; c1ccc(-c2ccc(N(c3ccc(-c4cccc5oc6ccccc6c45)cc3)c3cccc4c3ccc3ccccc34)c3c2sc2ccccc23)cc1 ; c1ccc(-c2ccc(N(c3ccccc3)c3ccc4c(ccc5ccccc54)c3)c3c2sc2ccccc23)cc1] 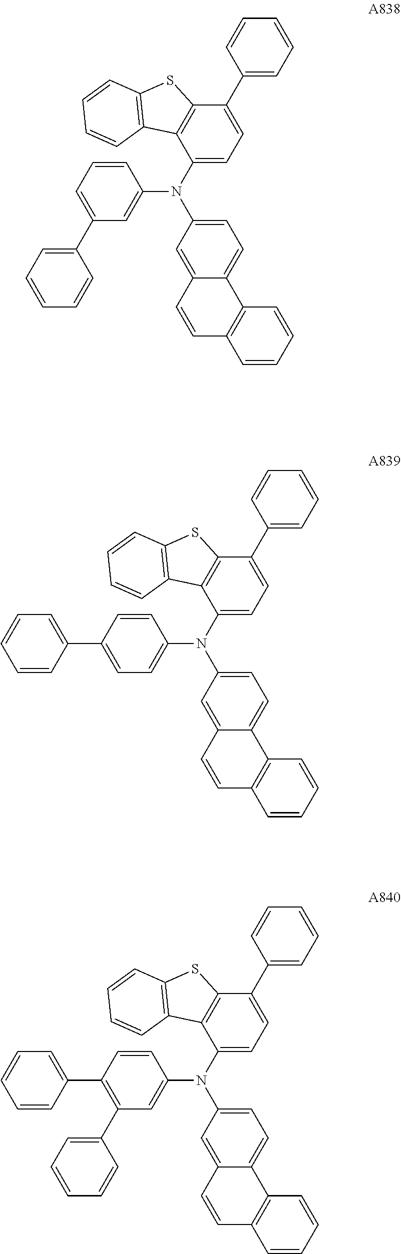 [SMILES: c1ccc(-c2cccc(N(c3ccc4c(ccc5ccccc54)c3)c3ccc(-c4ccccc4)c4sc5ccccc5c34)c2)cc1 ; c1ccc(-c2ccc(N(c3ccc4c(ccc5ccccc54)c3)c3ccc(-c4ccccc4)c4sc5ccccc5c34)cc2)cc1 ; c1ccc(-c2ccc(N(c3ccc4c(ccc5ccccc54)c3)c3ccc(-c4ccccc4)c4sc5ccccc5c34)cc2-c2ccccc2)cc1] 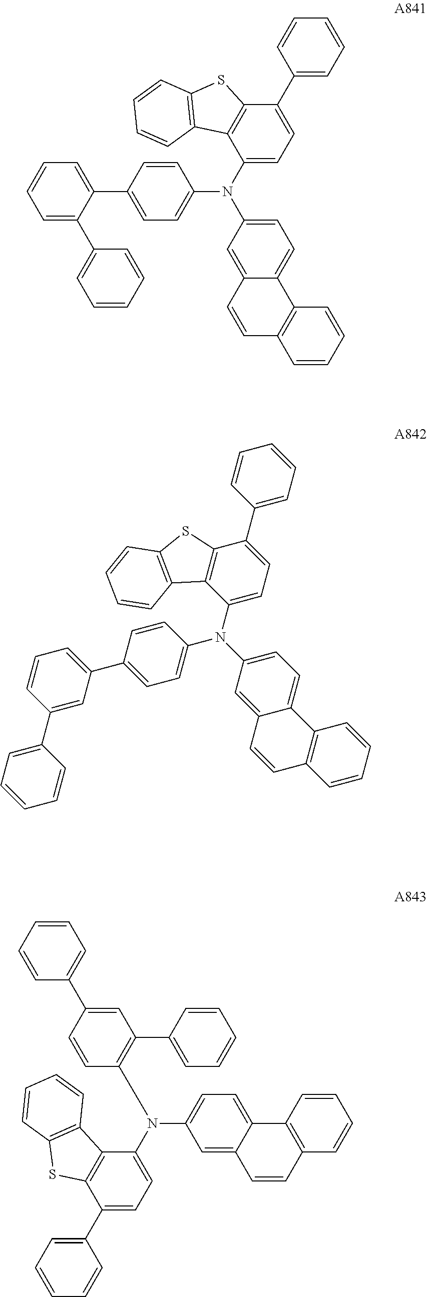 [SMILES: c1ccc(-c2ccccc2-c2ccc(N(c3ccc4c(ccc5ccccc54)c3)c3ccc(-c4ccccc4)c4sc5ccccc5c34)cc2)cc1 ; c1ccc(-c2cccc(-c3ccc(N(c4ccc5c(ccc6ccccc65)c4)c4ccc(-c5ccccc5)c5sc6ccccc6c45)cc3)c2)cc1 ; c1ccc(-c2ccc(N(c3ccc4c(ccc5ccccc54)c3)c3ccc(-c4ccccc4)c4sc5ccccc5c34)c(-c3ccccc3)c2)cc1] 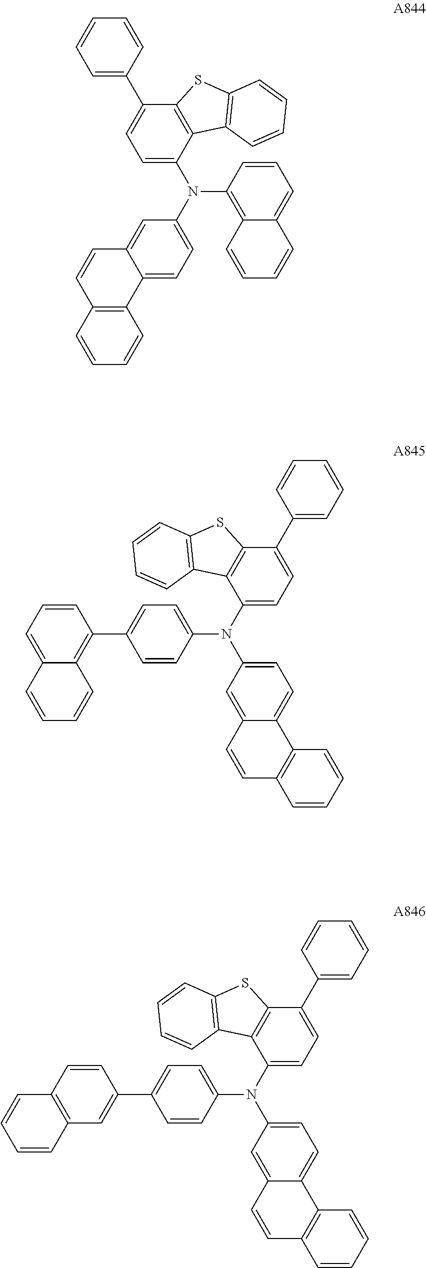 [SMILES: c1ccc(-c2ccc(N(c3ccc4c(ccc5ccccc54)c3)c3cccc4ccccc34)c3c2sc2ccccc23)cc1 ; c1ccc(-c2ccc(N(c3ccc(-c4cccc5ccccc45)cc3)c3ccc4c(ccc5ccccc54)c3)c3c2sc2ccccc23)cc1 ; c1ccc(-c2ccc(N(c3ccc(-c4ccc5ccccc5c4)cc3)c3ccc4c(ccc5ccccc54)c3)c3c2sc2ccccc23)cc1] 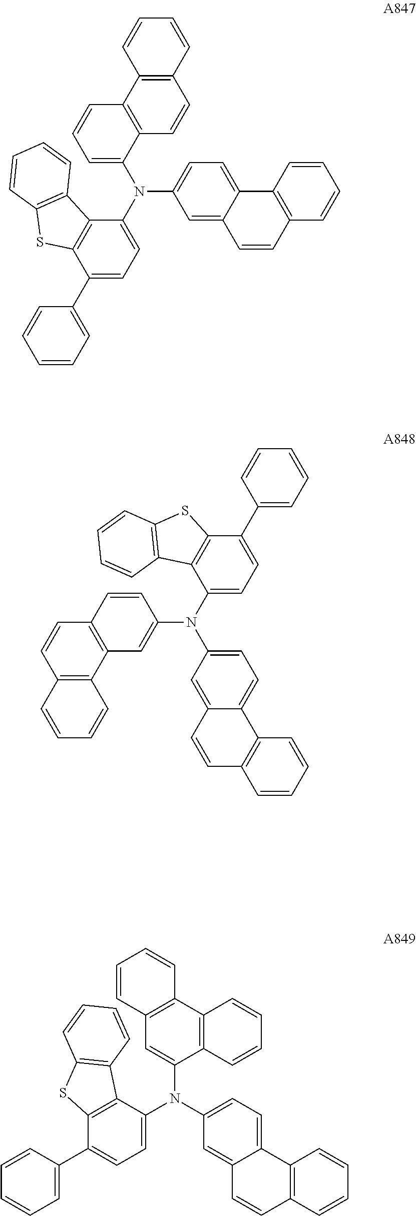 [SMILES: c1ccc(-c2ccc(N(c3ccc4c(ccc5ccccc54)c3)c3cccc4c3ccc3ccccc34)c3c2sc2ccccc23)cc1 ; c1ccc(-c2ccc(N(c3ccc4c(ccc5ccccc54)c3)c3ccc4ccc5ccccc5c4c3)c3c2sc2ccccc23)cc1 ; c1ccc(-c2ccc(N(c3ccc4c(ccc5ccccc54)c3)c3cc4ccccc4c4ccccc34)c3c2sc2ccccc23)cc1] 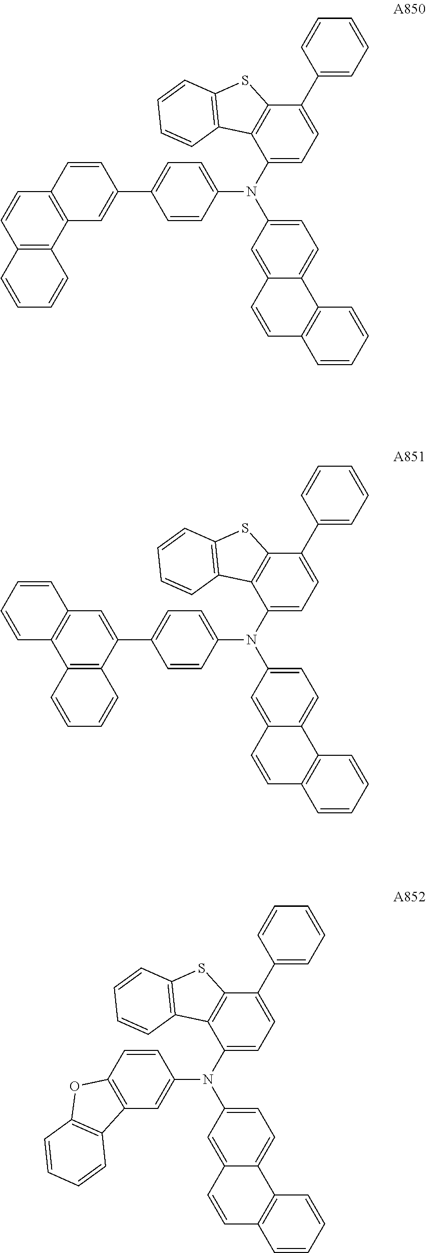 [SMILES: c1ccc(-c2ccc(N(c3ccc(-c4ccc5ccc6ccccc6c5c4)cc3)c3ccc4c(ccc5ccccc54)c3)c3c2sc2ccccc23)cc1 ; c1ccc(-c2ccc(N(c3ccc(-c4cc5ccccc5c5ccccc45)cc3)c3ccc4c(ccc5ccccc54)c3)c3c2sc2ccccc23)cc1 ; c1ccc(-c2ccc(N(c3ccc4c(ccc5ccccc54)c3)c3ccc4oc5ccccc5c4c3)c3c2sc2ccccc23)cc1] 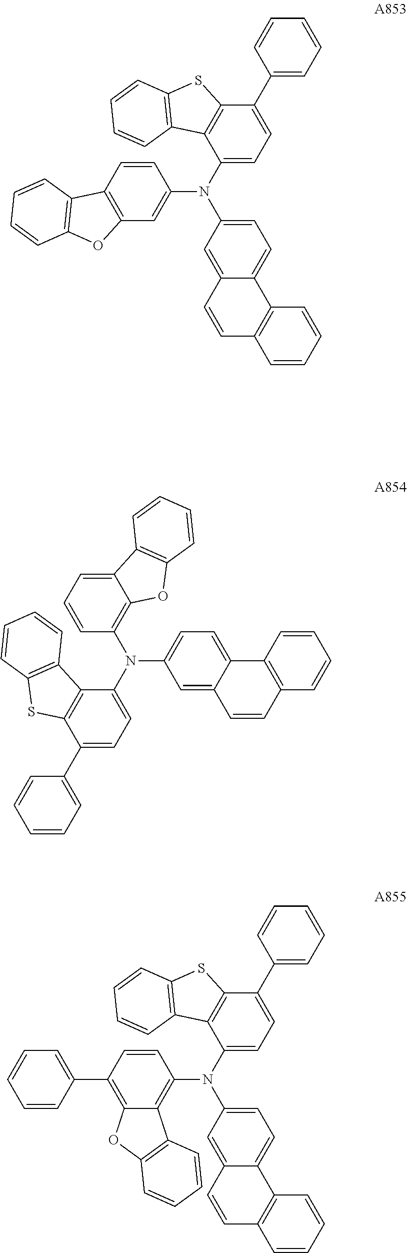 [SMILES: c1ccc(-c2ccc(N(c3ccc4c(ccc5ccccc54)c3)c3ccc4c(c3)oc3ccccc34)c3c2sc2ccccc23)cc1 ; c1ccc(-c2ccc(N(c3ccc4c(ccc5ccccc54)c3)c3cccc4c3oc3ccccc34)c3c2sc2ccccc23)cc1 ; c1ccc(-c2ccc(N(c3ccc4c(ccc5ccccc54)c3)c3ccc(-c4ccccc4)c4sc5ccccc5c34)c3c2oc2ccccc23)cc1] 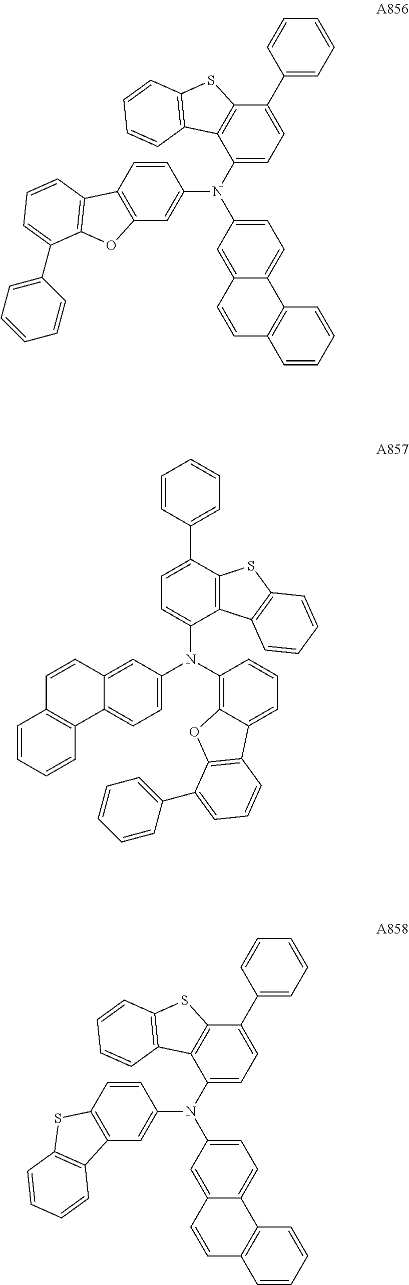 [SMILES: c1ccc(-c2cccc3c2oc2cc(N(c4ccc5c(ccc6ccccc65)c4)c4ccc(-c5ccccc5)c5sc6ccccc6c45)ccc23)cc1 ; c1ccc(-c2cccc3c2oc2c(N(c4ccc5c(ccc6ccccc65)c4)c4ccc(-c5ccccc5)c5sc6ccccc6c45)cccc23)cc1 ; c1ccc(-c2ccc(N(c3ccc4c(ccc5ccccc54)c3)c3ccc4sc5ccccc5c4c3)c3c2sc2ccccc23)cc1] 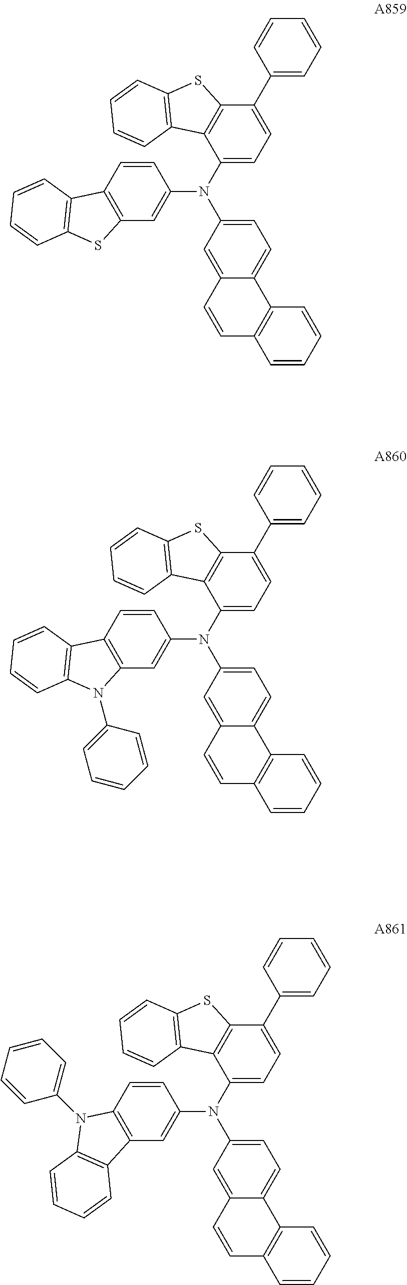 [SMILES: c1ccc(-c2ccc(N(c3ccc4c(ccc5ccccc54)c3)c3ccc4c(c3)sc3ccccc34)c3c2sc2ccccc23)cc1 ; c1ccc(-c2ccc(N(c3ccc4c(ccc5ccccc54)c3)c3ccc4c5ccccc5n(-c5ccccc5)c4c3)c3c2sc2ccccc23)cc1 ; c1ccc(-c2ccc(N(c3ccc4c(ccc5ccccc54)c3)c3ccc4c(c3)c3ccccc3n4-c3ccccc3)c3c2sc2ccccc23)cc1] 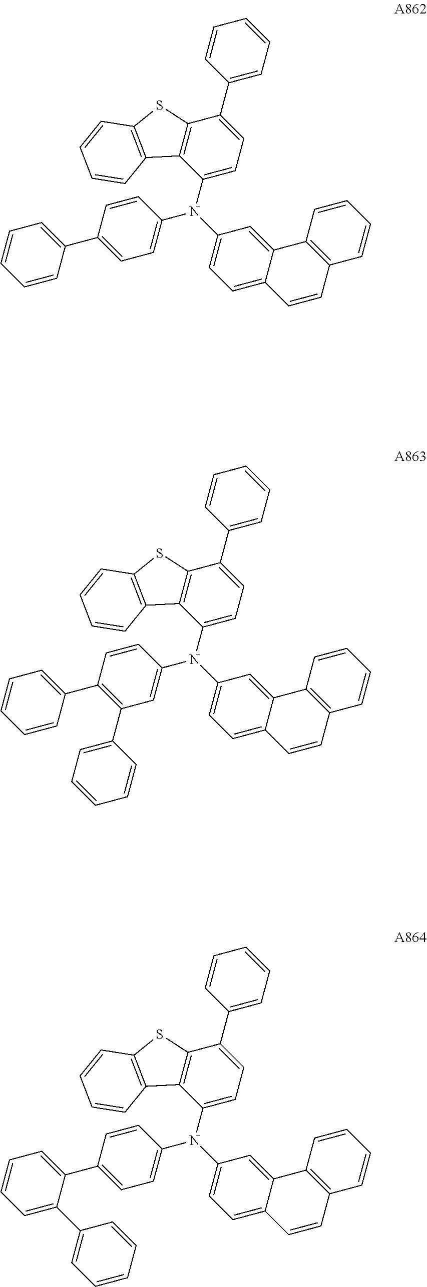 [SMILES: c1ccc(-c2ccc(N(c3ccc4ccc5ccccc5c4c3)c3ccc(-c4ccccc4)c4sc5ccccc5c34)cc2)cc1 ; c1ccc(-c2ccc(N(c3ccc4ccc5ccccc5c4c3)c3ccc(-c4ccccc4)c4sc5ccccc5c34)cc2-c2ccccc2)cc1 ; c1ccc(-c2ccccc2-c2ccc(N(c3ccc4ccc5ccccc5c4c3)c3ccc(-c4ccccc4)c4sc5ccccc5c34)cc2)cc1] 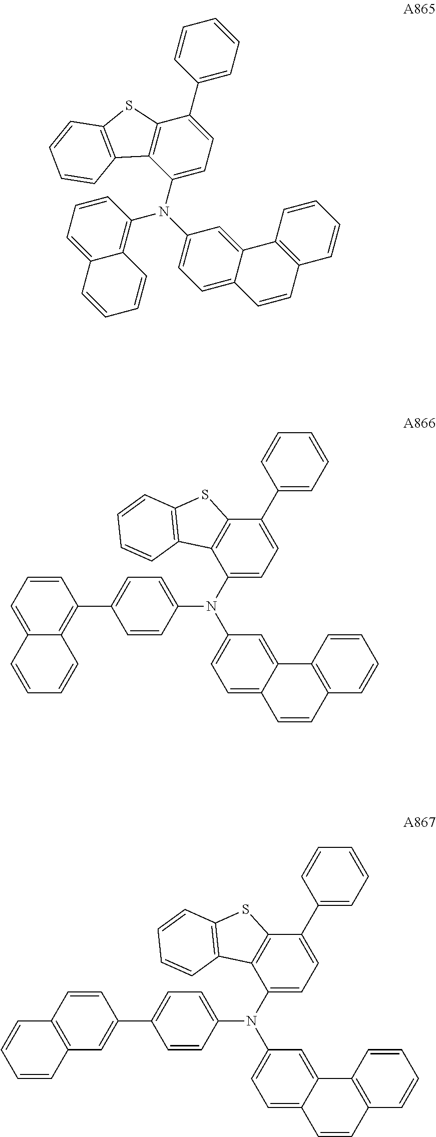 [SMILES: c1ccc(-c2ccc(N(c3ccc4ccc5ccccc5c4c3)c3cccc4ccccc34)c3c2sc2ccccc23)cc1 ; c1ccc(-c2ccc(N(c3ccc(-c4cccc5ccccc45)cc3)c3ccc4ccc5ccccc5c4c3)c3c2sc2ccccc23)cc1 ; c1ccc(-c2ccc(N(c3ccc(-c4ccc5ccccc5c4)cc3)c3ccc4ccc5ccccc5c4c3)c3c2sc2ccccc23)cc1] 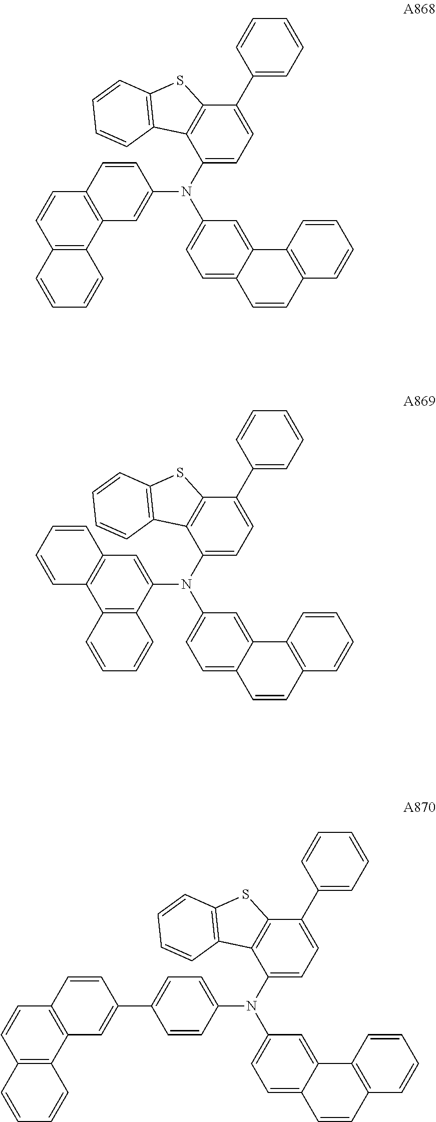 [SMILES: c1ccc(-c2ccc(N(c3ccc4ccc5ccccc5c4c3)c3ccc4ccc5ccccc5c4c3)c3c2sc2ccccc23)cc1 ; c1ccc(-c2ccc(N(c3ccc4ccc5ccccc5c4c3)c3cc4ccccc4c4ccccc34)c3c2sc2ccccc23)cc1 ; c1ccc(-c2ccc(N(c3ccc(-c4ccc5ccc6ccccc6c5c4)cc3)c3ccc4ccc5ccccc5c4c3)c3c2sc2ccccc23)cc1] 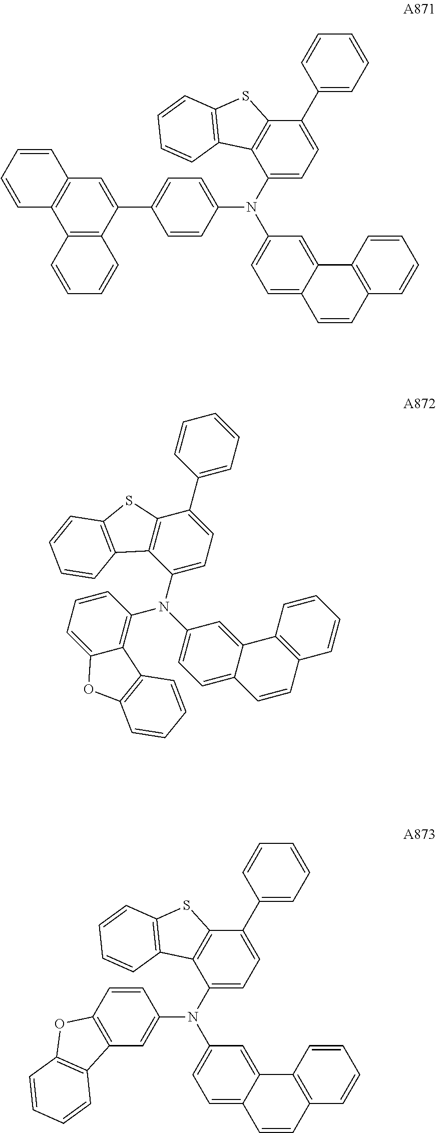 [SMILES: c1ccc(-c2ccc(N(c3ccc(-c4cc5ccccc5c5ccccc45)cc3)c3ccc4ccc5ccccc5c4c3)c3c2sc2ccccc23)cc1 ; c1ccc(-c2ccc(N(c3ccc4ccc5ccccc5c4c3)c3cccc4oc5ccccc5c34)c3c2sc2ccccc23)cc1 ; c1ccc(-c2ccc(N(c3ccc4ccc5ccccc5c4c3)c3ccc4oc5ccccc5c4c3)c3c2sc2ccccc23)cc1] 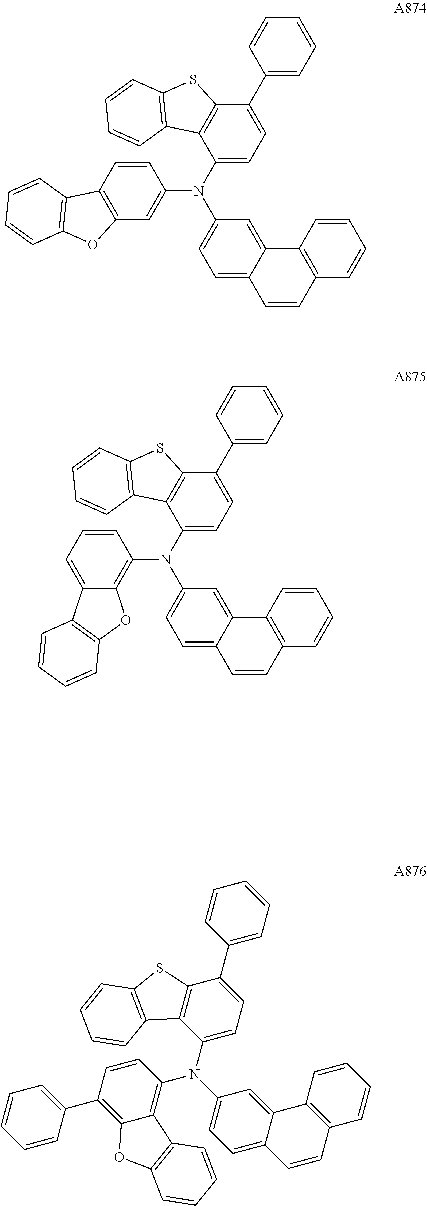 [SMILES: c1ccc(-c2ccc(N(c3ccc4c(c3)oc3ccccc34)c3ccc4ccc5ccccc5c4c3)c3c2sc2ccccc23)cc1 ; c1ccc(-c2ccc(N(c3ccc4ccc5ccccc5c4c3)c3cccc4c3oc3ccccc34)c3c2sc2ccccc23)cc1 ; c1ccc(-c2ccc(N(c3ccc4ccc5ccccc5c4c3)c3ccc(-c4ccccc4)c4sc5ccccc5c34)c3c2oc2ccccc23)cc1] 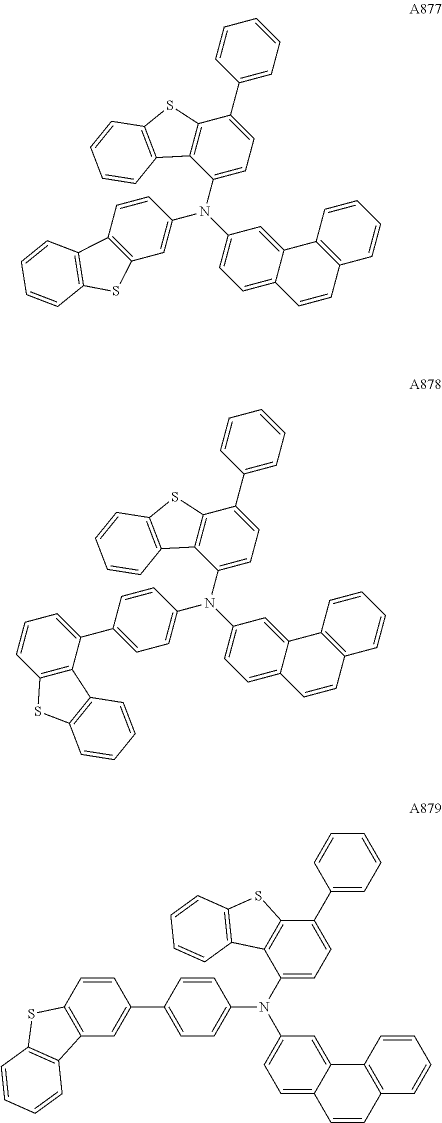 [SMILES: c1ccc(-c2ccc(N(c3ccc4c(c3)sc3ccccc34)c3ccc4ccc5ccccc5c4c3)c3c2sc2ccccc23)cc1 ; c1ccc(-c2ccc(N(c3ccc(-c4cccc5sc6ccccc6c45)cc3)c3ccc4ccc5ccccc5c4c3)c3c2sc2ccccc23)cc1 ; c1ccc(-c2ccc(N(c3ccc(-c4ccc5sc6ccccc6c5c4)cc3)c3ccc4ccc5ccccc5c4c3)c3c2sc2ccccc23)cc1] 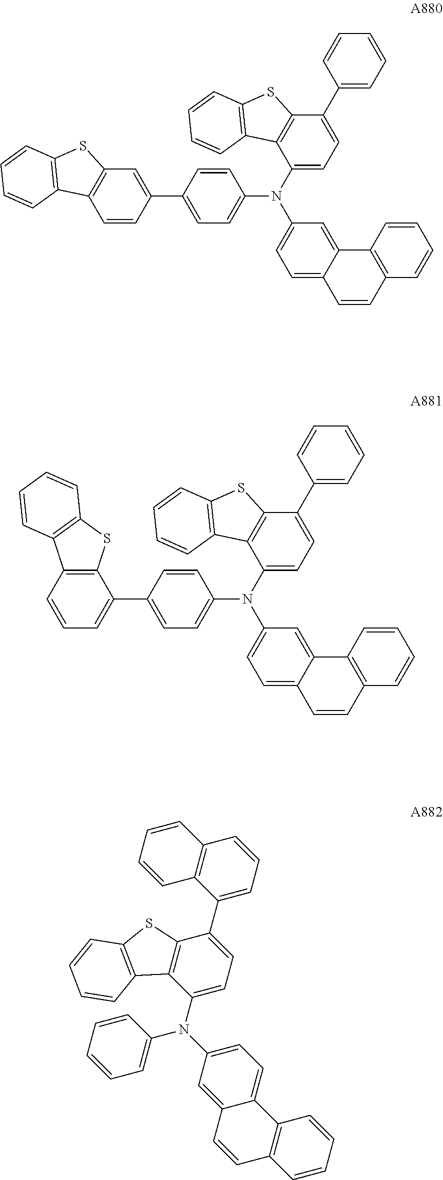 [SMILES: c1ccc(-c2ccc(N(c3ccc(-c4ccc5c(c4)sc4ccccc45)cc3)c3ccc4ccc5ccccc5c4c3)c3c2sc2ccccc23)cc1 ; c1ccc(-c2ccc(N(c3ccc(-c4cccc5c4sc4ccccc45)cc3)c3ccc4ccc5ccccc5c4c3)c3c2sc2ccccc23)cc1 ; c1ccc(N(c2ccc3c(ccc4ccccc43)c2)c2ccc(-c3cccc4ccccc34)c3sc4ccccc4c23)cc1] 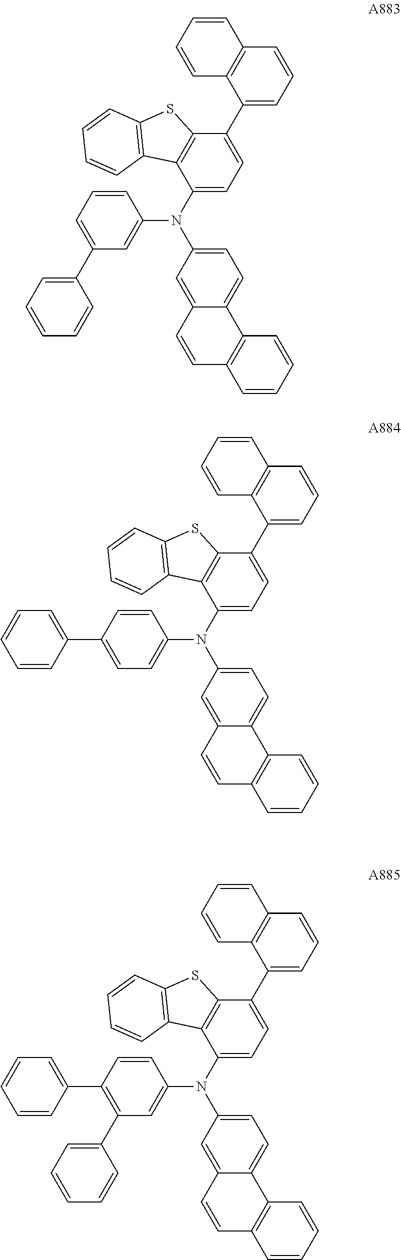 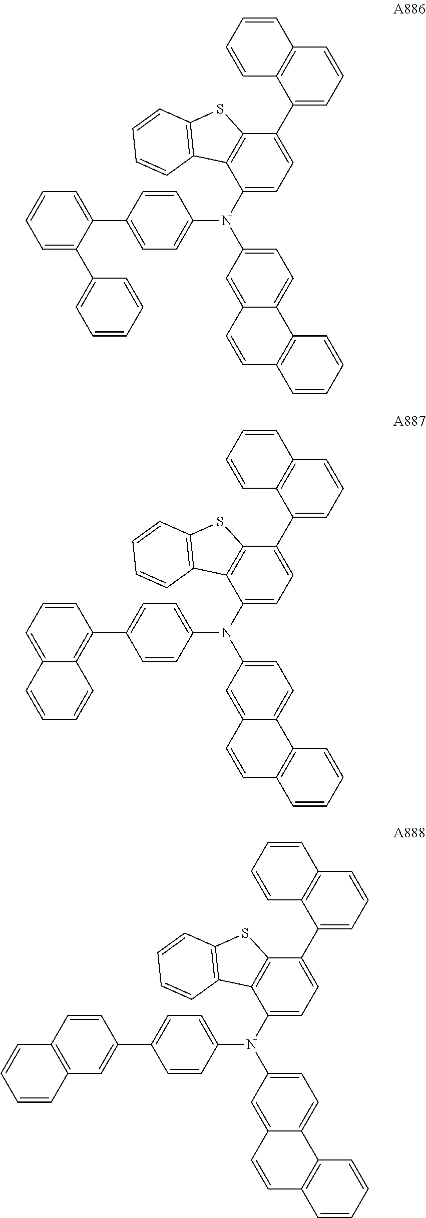 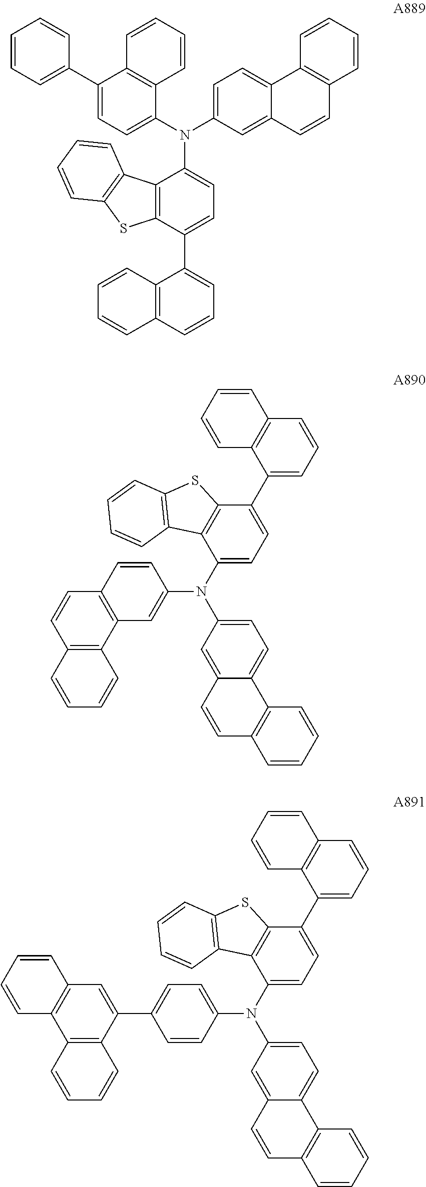 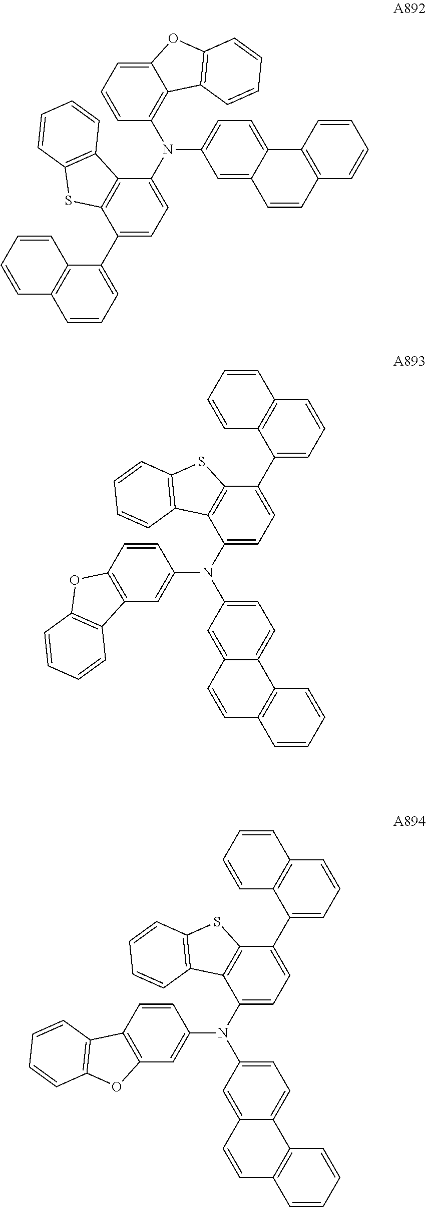 [SMILES: c1ccc2c(-c3ccc(N(c4ccc5c(ccc6ccccc65)c4)c4cccc5oc6ccccc6c45)c4c3sc3ccccc34)cccc2c1 ; c1ccc2c(-c3ccc(N(c4ccc5c(ccc6ccccc65)c4)c4ccc5oc6ccccc6c5c4)c4c3sc3ccccc34)cccc2c1 ; c1ccc2c(-c3ccc(N(c4ccc5c(ccc6ccccc65)c4)c4ccc5c(c4)oc4ccccc45)c4c3sc3ccccc34)cccc2c1] 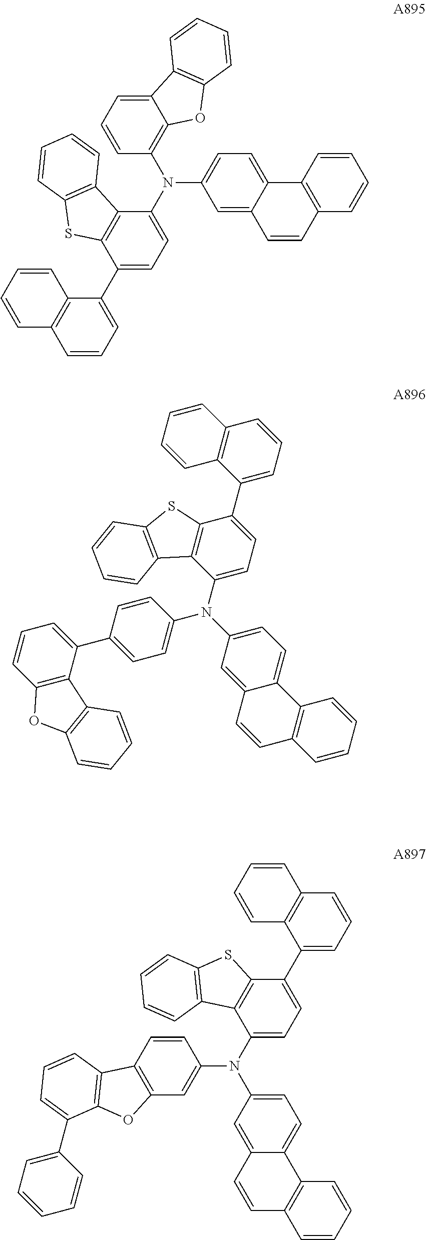 [SMILES: c1ccc2c(-c3ccc(N(c4ccc5c(ccc6ccccc65)c4)c4cccc5c4oc4ccccc45)c4c3sc3ccccc34)cccc2c1 ; c1ccc2c(-c3ccc(N(c4ccc(-c5cccc6oc7ccccc7c56)cc4)c4ccc5c(ccc6ccccc65)c4)c4c3sc3ccccc34)cccc2c1 ; c1ccc(-c2cccc3c2oc2cc(N(c4ccc5c(ccc6ccccc65)c4)c4ccc(-c5cccc6ccccc56)c5sc6ccccc6c45)ccc23)cc1] 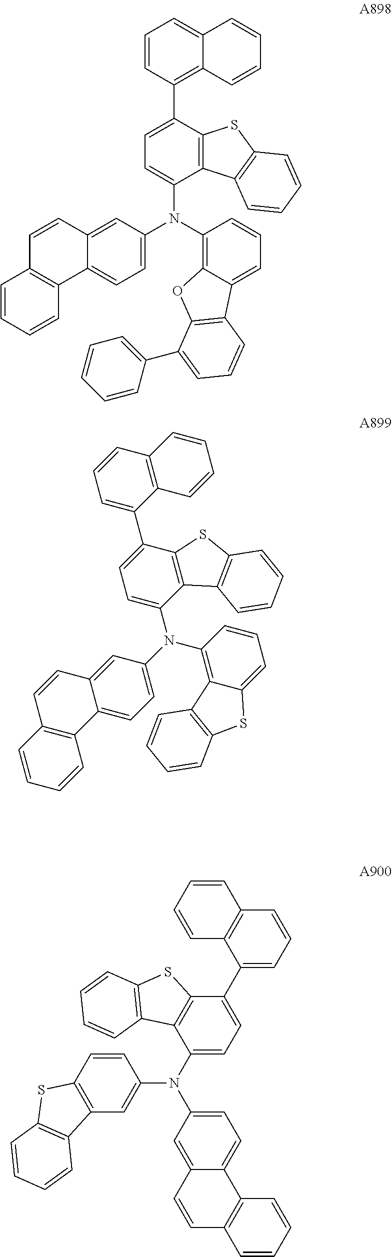 [SMILES: c1ccc(-c2cccc3c2oc2c(N(c4ccc5c(ccc6ccccc65)c4)c4ccc(-c5cccc6ccccc56)c5sc6ccccc6c45)cccc23)cc1 ; c1ccc2c(-c3ccc(N(c4ccc5c(ccc6ccccc65)c4)c4cccc5sc6ccccc6c45)c4c3sc3ccccc34)cccc2c1 ; c1ccc2c(-c3ccc(N(c4ccc5c(ccc6ccccc65)c4)c4ccc5sc6ccccc6c5c4)c4c3sc3ccccc34)cccc2c1] 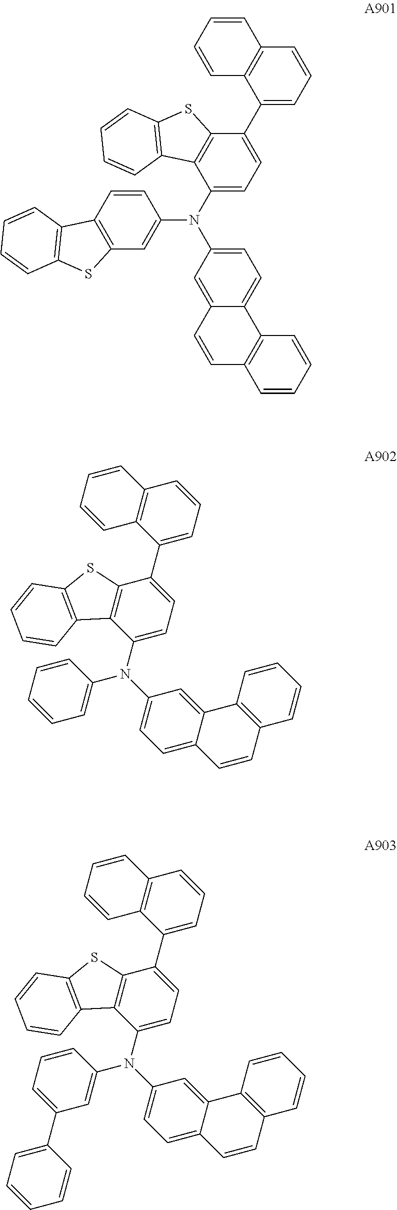 [SMILES: c1ccc2c(-c3ccc(N(c4ccc5c(ccc6ccccc65)c4)c4ccc5c(c4)sc4ccccc45)c4c3sc3ccccc34)cccc2c1 ; c1ccc(N(c2ccc3ccc4ccccc4c3c2)c2ccc(-c3cccc4ccccc34)c3sc4ccccc4c23)cc1 ; c1ccc(-c2cccc(N(c3ccc4ccc5ccccc5c4c3)c3ccc(-c4cccc5ccccc45)c4sc5ccccc5c34)c2)cc1] 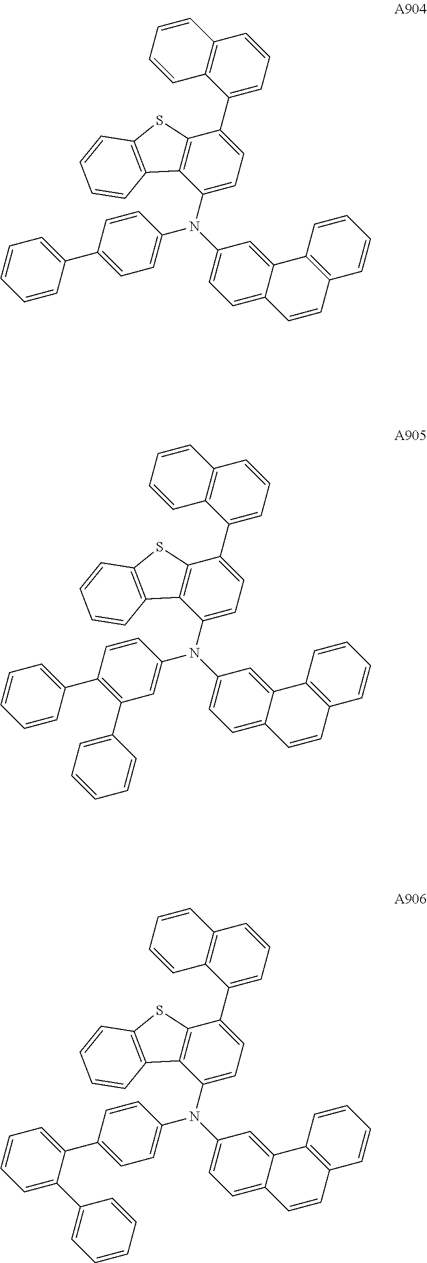 [SMILES: c1ccc(-c2ccc(N(c3ccc4ccc5ccccc5c4c3)c3ccc(-c4cccc5ccccc45)c4sc5ccccc5c34)cc2)cc1 ; c1ccc(-c2ccc(N(c3ccc4ccc5ccccc5c4c3)c3ccc(-c4cccc5ccccc45)c4sc5ccccc5c34)cc2-c2ccccc2)cc1 ; c1ccc(-c2ccccc2-c2ccc(N(c3ccc4ccc5ccccc5c4c3)c3ccc(-c4cccc5ccccc45)c4sc5ccccc5c34)cc2)cc1] 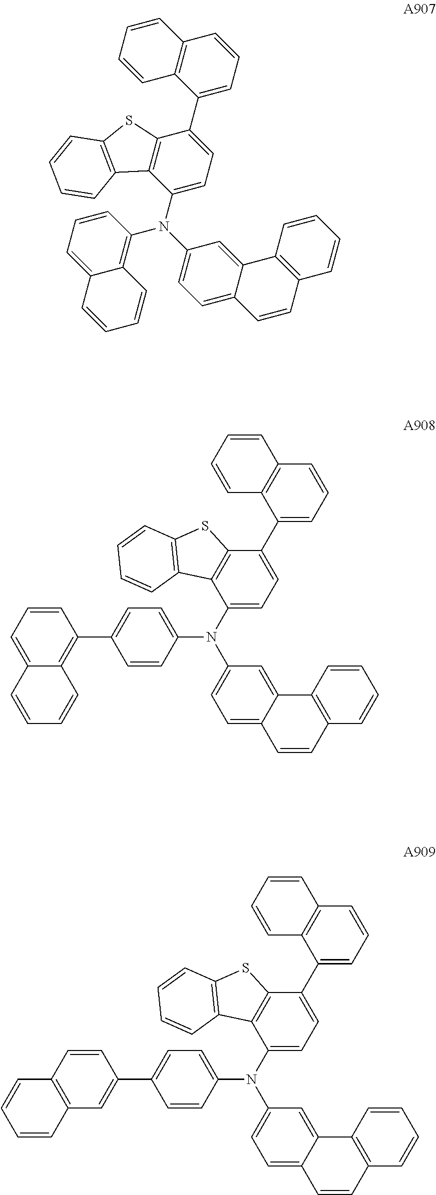 [SMILES: c1ccc2c(-c3ccc(N(c4ccc5ccc6ccccc6c5c4)c4cccc5ccccc45)c4c3sc3ccccc34)cccc2c1 ; c1ccc2c(-c3ccc(N(c4ccc5ccc6ccccc6c5c4)c4ccc(-c5cccc6ccccc56)c5sc6ccccc6c45)cc3)cccc2c1 ; c1ccc2cc(-c3ccc(N(c4ccc5ccc6ccccc6c5c4)c4ccc(-c5cccc6ccccc56)c5sc6ccccc6c45)cc3)ccc2c1] 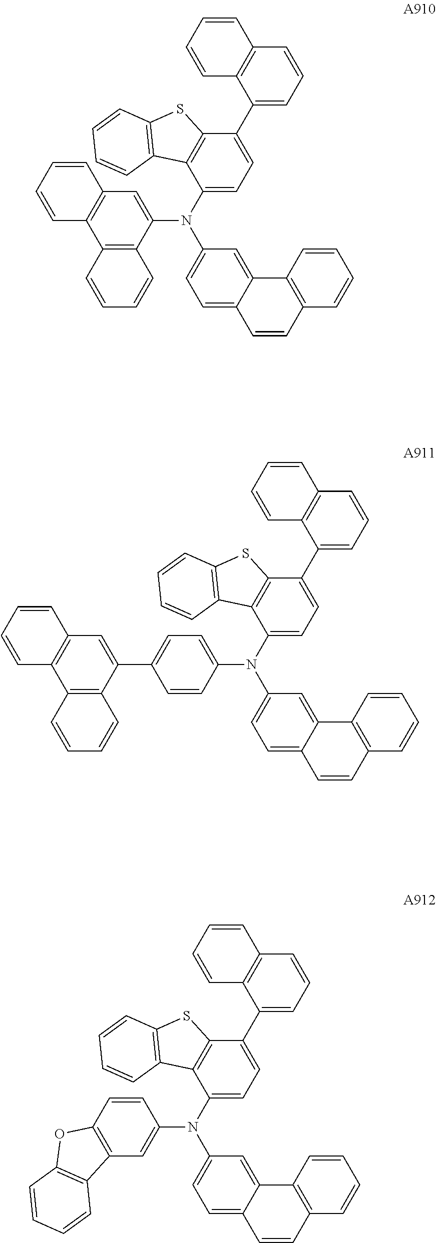 [SMILES: c1ccc2c(-c3ccc(N(c4ccc5ccc6ccccc6c5c4)c4cc5ccccc5c5ccccc45)c4c3sc3ccccc34)cccc2c1 ; c1ccc2c(-c3ccc(N(c4ccc(-c5cc6ccccc6c6ccccc56)cc4)c4ccc5ccc6ccccc6c5c4)c4c3sc3ccccc34)cccc2c1 ; c1ccc2c(-c3ccc(N(c4ccc5ccc6ccccc6c5c4)c4ccc5oc6ccccc6c5c4)c4c3sc3ccccc34)cccc2c1] 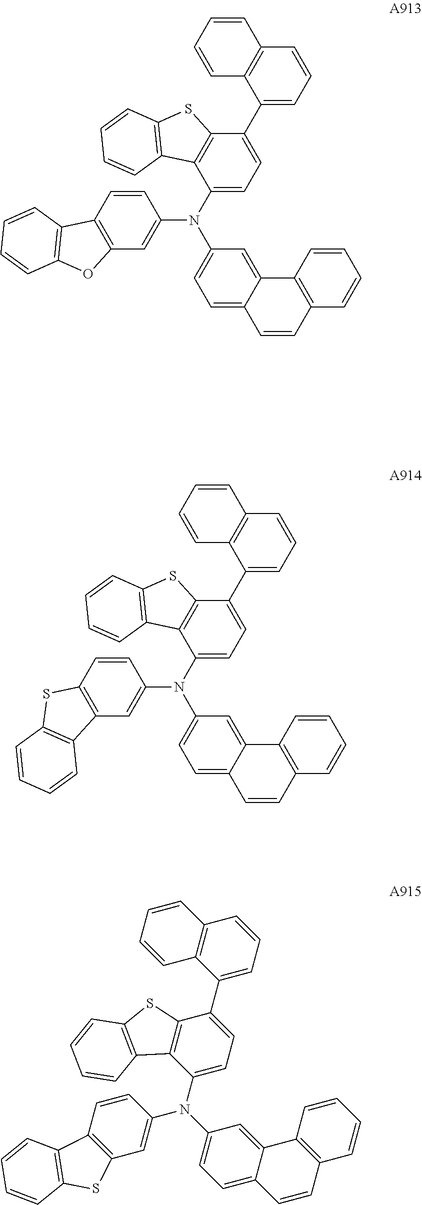 [SMILES: c1ccc2c(-c3ccc(N(c4ccc5c(c4)oc4ccccc45)c4ccc5ccc6ccccc6c5c4)c4c3sc3ccccc34)cccc2c1 ; c1ccc2c(-c3ccc(N(c4ccc5ccc6ccccc6c5c4)c4ccc5sc6ccccc6c5c4)c4c3sc3ccccc34)cccc2c1 ; c1ccc2c(-c3ccc(N(c4ccc5c(c4)sc4ccccc45)c4ccc5ccc6ccccc6c5c4)c4c3sc3ccccc34)cccc2c1] 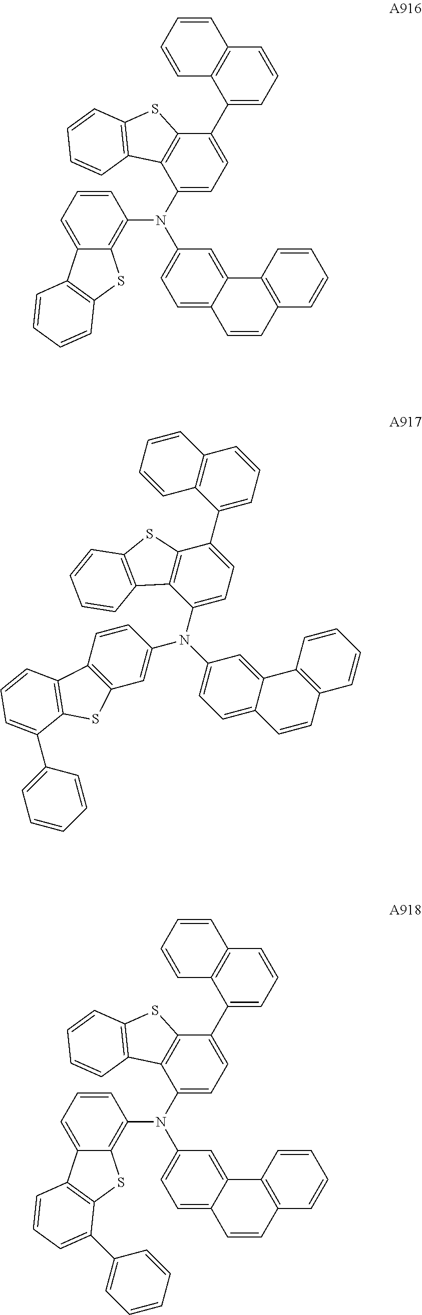 [SMILES: c1ccc2c(-c3ccc(N(c4ccc5ccc6ccccc6c5c4)c4cccc5c4sc4ccccc45)c4c3sc3ccccc34)cccc2c1 ; c1ccc(-c2cccc3c2sc2cc(N(c4ccc5ccc6ccccc6c5c4)c4ccc(-c5cccc6ccccc56)c5sc6ccccc6c45)ccc23)cc1 ; c1ccc(-c2cccc3c2sc2c(N(c4ccc5ccc6ccccc6c5c4)c4ccc(-c5cccc6ccccc56)c5sc6ccccc6c45)cccc23)cc1] 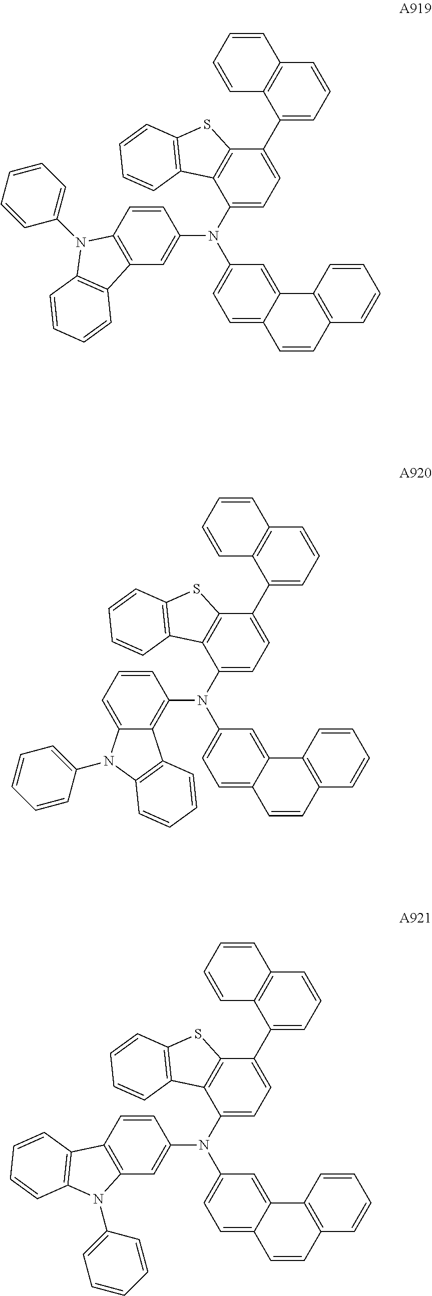 [SMILES: c1ccc(-n2c3ccccc3c3cc(N(c4ccc5ccc6ccccc6c5c4)c4ccc(-c5cccc6ccccc56)c5sc6ccccc6c45)ccc32)cc1 ; c1ccc(-n2c3ccccc3c3c(N(c4ccc5ccc6ccccc6c5c4)c4ccc(-c5cccc6ccccc56)c5sc6ccccc6c45)cccc32)cc1 ; c1ccc(-n2c3ccccc3c3ccc(N(c4ccc5ccc6ccccc6c5c4)c4ccc(-c5cccc6ccccc56)c5sc6ccccc6c45)cc32)cc1] 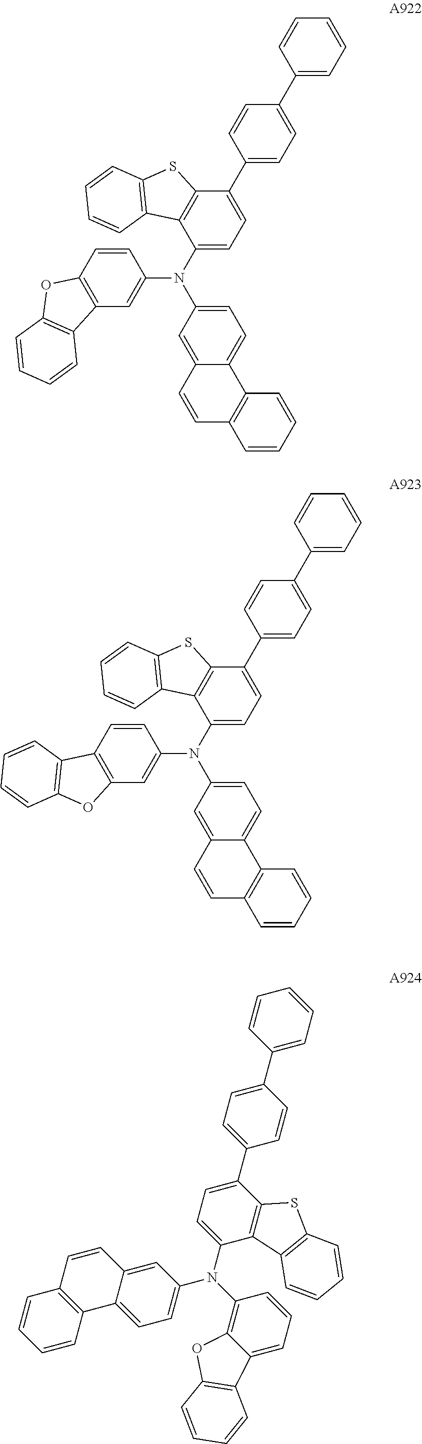 [SMILES: c1ccc(-c2ccc(-c3ccc(N(c4ccc5c(ccc6ccccc65)c4)c4ccc5oc6ccccc6c5c4)c4c3sc3ccccc34)cc2)cc1 ; c1ccc(-c2ccc(-c3ccc(N(c4ccc5c(ccc6ccccc65)c4)c4ccc5c(c4)oc4ccccc45)c4c3sc3ccccc34)cc2)cc1 ; c1ccc(-c2ccc(-c3ccc(N(c4ccc5c(ccc6ccccc65)c4)c4cccc5c4oc4ccccc45)c4c3sc3ccccc34)cc2)cc1] 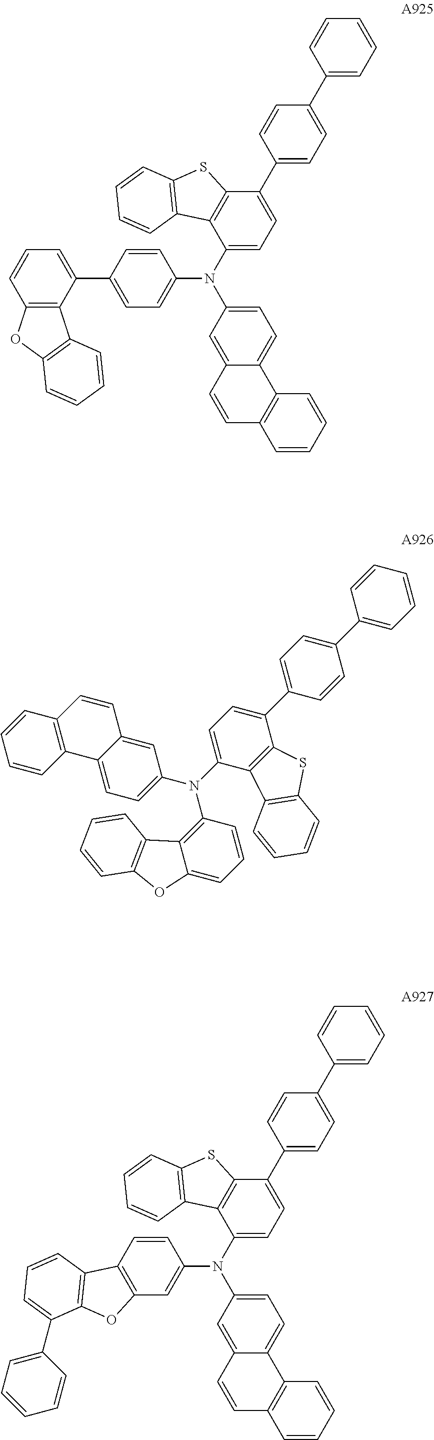 [SMILES: c1ccc(-c2ccc(-c3ccc(N(c4ccc(-c5cccc6oc7ccccc7c56)cc4)c4ccc5c(ccc6ccccc65)c4)c4c3sc3ccccc34)cc2)cc1 ; c1ccc(-c2ccc(-c3ccc(N(c4ccc5c(ccc6ccccc65)c4)c4cccc5oc6ccccc6c45)c4c3sc3ccccc34)cc2)cc1 ; c1ccc(-c2ccc(-c3ccc(N(c4ccc5c(ccc6ccccc65)c4)c4ccc5c(c4)oc4c(-c6ccccc6)cccc45)c4c3sc3ccccc34)cc2)cc1] 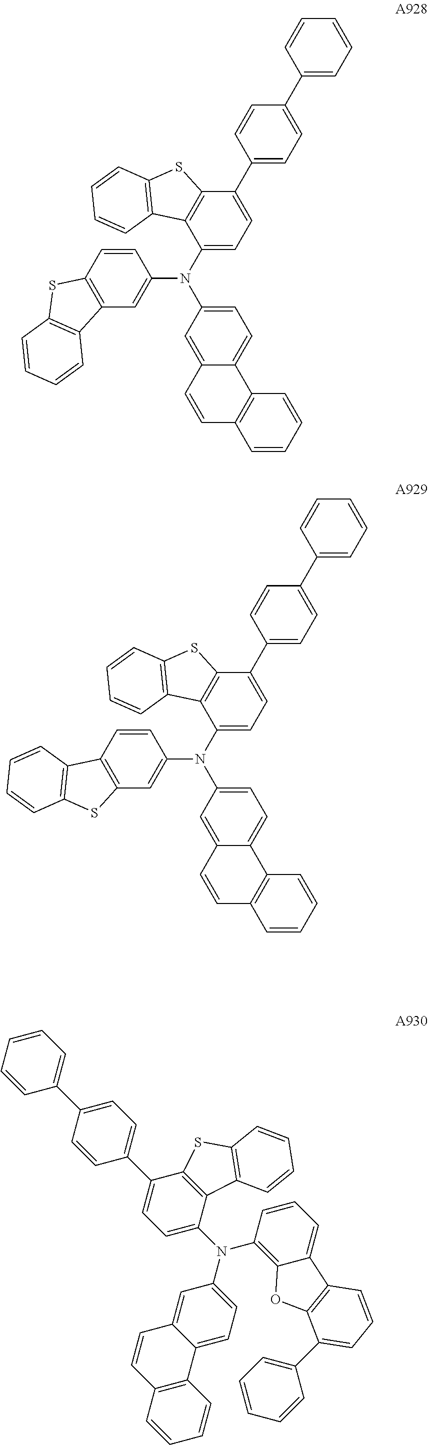 [SMILES: c1ccc(-c2ccc(-c3ccc(N(c4ccc5c(ccc6ccccc65)c4)c4ccc5sc6ccccc6c5c4)c4c3sc3ccccc34)cc2)cc1 ; c1ccc(-c2ccc(-c3ccc(N(c4ccc5c(ccc6ccccc65)c4)c4ccc5c(c4)sc4ccccc45)c4c3sc3ccccc34)cc2)cc1 ; c1ccc(-c2ccc(-c3ccc(N(c4ccc5c(ccc6ccccc65)c4)c4cccc5c4oc4c(-c6ccccc6)cccc45)c4c3sc3ccccc34)cc2)cc1] 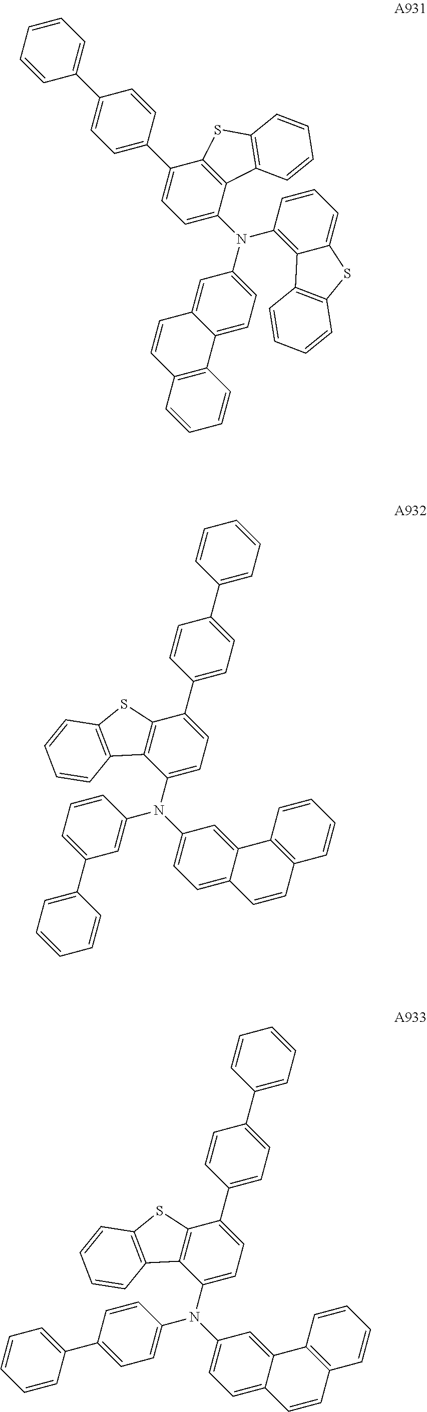 [SMILES: c1ccc(-c2ccc(-c3ccc(N(c4ccc5c(ccc6ccccc65)c4)c4cccc5sc6ccccc6c45)c4c3sc3ccccc34)cc2)cc1 ; c1ccc(-c2ccc(-c3ccc(N(c4cccc(-c5ccccc5)c4)c4ccc5ccc6ccccc6c5c4)c4c3sc3ccccc34)cc2)cc1 ; c1ccc(-c2ccc(-c3ccc(N(c4ccc(-c5ccccc5)cc4)c4ccc5ccc6ccccc6c5c4)c4c3sc3ccccc34)cc2)cc1] 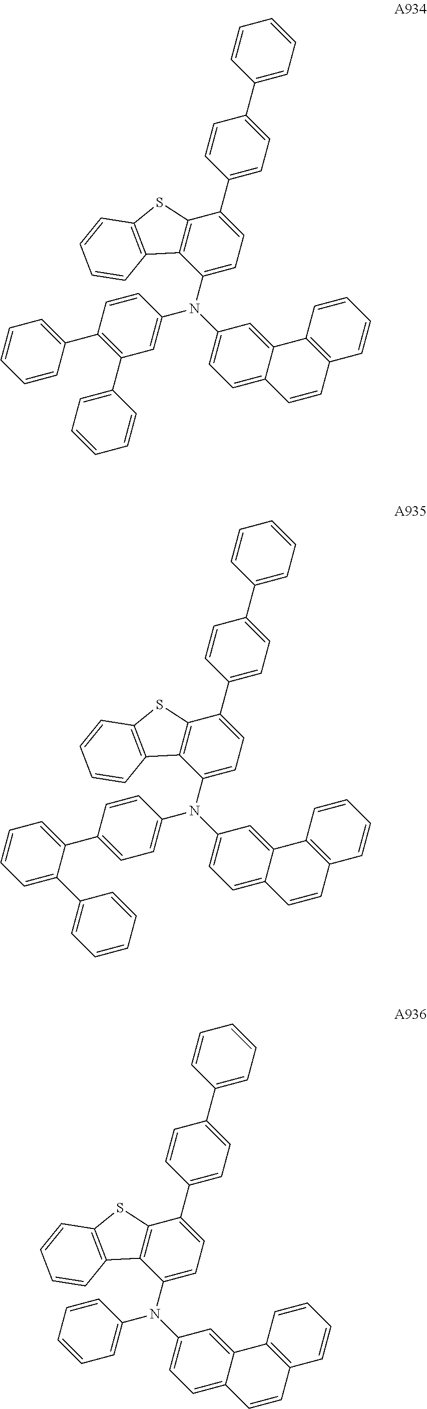 [SMILES: c1ccc(-c2ccc(-c3ccc(N(c4ccc(-c5ccccc5)c(-c5ccccc5)c4)c4ccc5ccc6ccccc6c5c4)c4c3sc3ccccc34)cc2)cc1 ; c1ccc(-c2ccc(-c3ccc(N(c4ccc(-c5ccccc5-c5ccccc5)cc4)c4ccc5ccc6ccccc6c5c4)c4c3sc3ccccc34)cc2)cc1 ; c1ccc(-c2ccc(-c3ccc(N(c4ccccc4)c4ccc5ccc6ccccc6c5c4)c4c3sc3ccccc34)cc2)cc1] 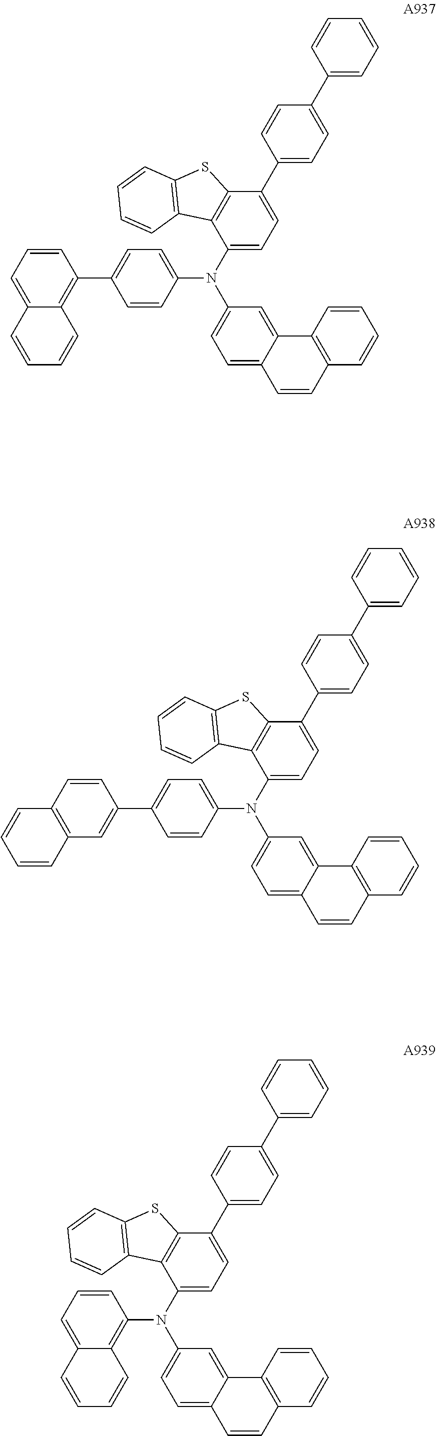 [SMILES: c1ccc(-c2ccc(-c3ccc(N(c4ccc(-c5cccc6ccccc56)cc4)c4ccc5ccc6ccccc6c5c4)c4c3sc3ccccc34)cc2)cc1 ; c1ccc(-c2ccc(-c3ccc(N(c4ccc(-c5ccc6ccccc6c5)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5ccccc45)c4c3sc3ccccc34)cc2)cc1] 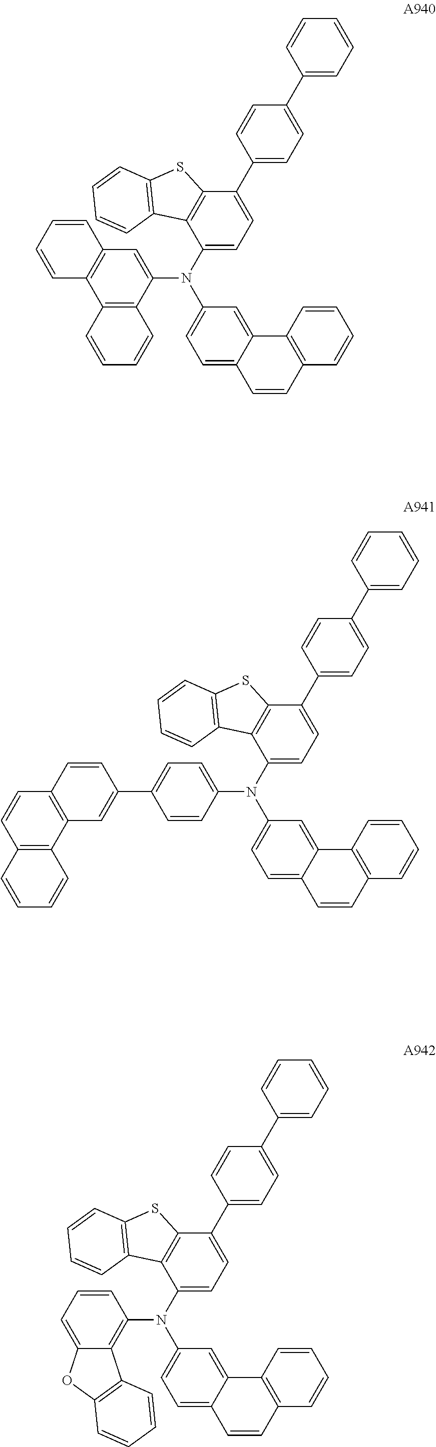 [SMILES: c1ccc(-c2ccc(-c3ccc(N(c4ccc5ccc6ccccc6c5c4)c4cc5ccccc5c5ccccc45)c4c3sc3ccccc34)cc2)cc1 ; c1ccc(-c2ccc(-c3ccc(N(c4ccc(-c5ccc6ccc7ccccc7c6c5)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5oc6ccccc6c45)c4c3sc3ccccc34)cc2)cc1] 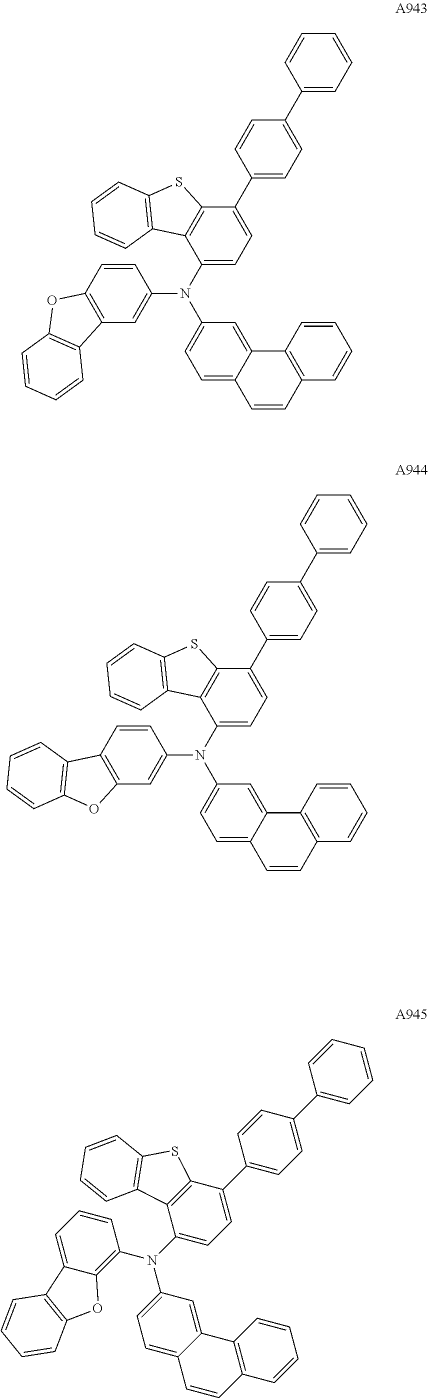 [SMILES: c1ccc(-c2ccc(-c3ccc(N(c4ccc5ccc6ccccc6c5c4)c4ccc5oc6ccccc6c5c4)c4c3sc3ccccc34)cc2)cc1 ; c1ccc(-c2ccc(-c3ccc(N(c4ccc5c(c4)oc4ccccc45)c4ccc5ccc6ccccc6c5c4)c4c3sc3ccccc34)cc2)cc1 ; c1ccc(-c2ccc(-c3ccc(N(c4ccc5ccc6ccccc6c5c4)c4cccc5c4oc4ccccc45)c4c3sc3ccccc34)cc2)cc1] 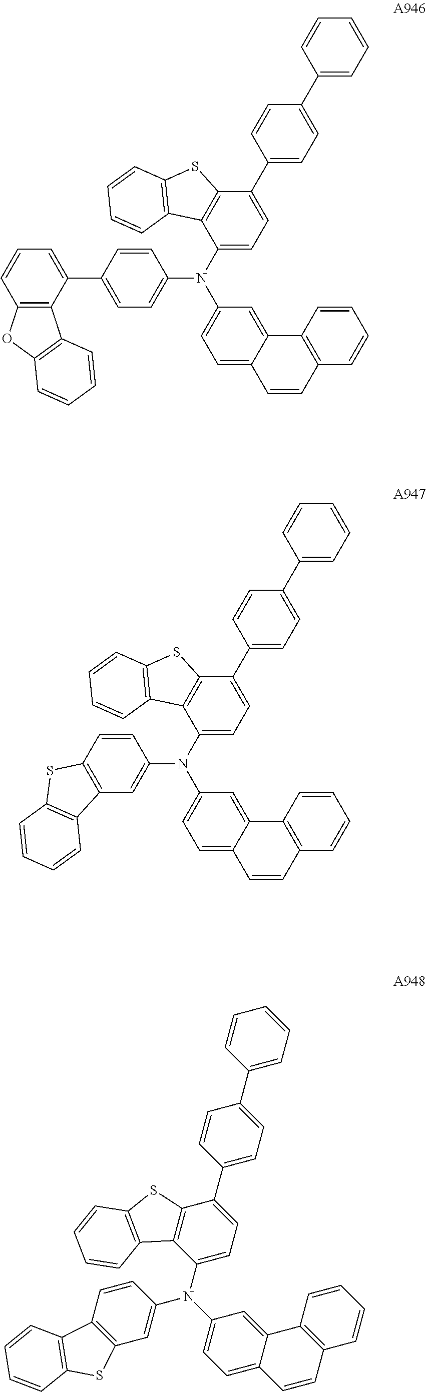 [SMILES: c1ccc(-c2ccc(-c3ccc(N(c4ccc(-c5cccc6oc7ccccc7c56)cc4)c4ccc5ccc6ccccc6c5c4)c4c3sc3ccccc34)cc2)cc1 ; c1ccc(-c2ccc(-c3ccc(N(c4ccc5ccc6ccccc6c5c4)c4ccc5sc6ccccc6c5c4)c4c3sc3ccccc34)cc2)cc1 ; c1ccc(-c2ccc(-c3ccc(N(c4ccc5c(c4)sc4ccccc45)c4ccc5ccc6ccccc6c5c4)c4c3sc3ccccc34)cc2)cc1] 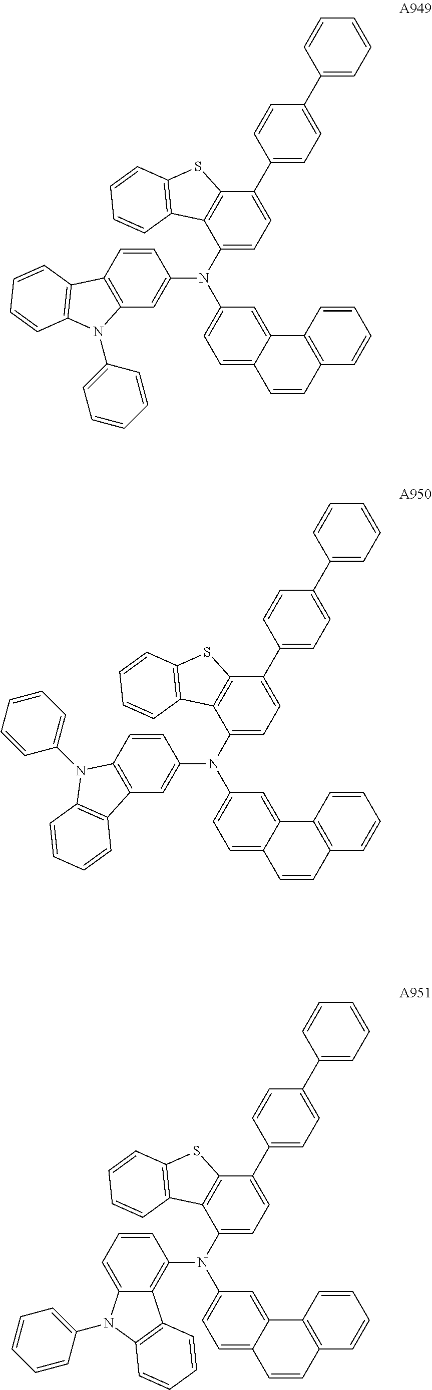 [SMILES: c1ccc(-c2ccc(-c3ccc(N(c4ccc5ccc6ccccc6c5c4)c4ccc5c6ccccc6n(-c6ccccc6)c5c4)c4c3sc3ccccc34)cc2)cc1 ; c1ccc(-c2ccc(-c3ccc(N(c4ccc5ccc6ccccc6c5c4)c4ccc5c(c4)c4ccccc4n5-c4ccccc4)c4c3sc3ccccc34)cc2)cc1 ; c1ccc(-c2ccc(-c3ccc(N(c4ccc5ccc6ccccc6c5c4)c4cccc5c4c4ccccc4n5-c4ccccc4)c4c3sc3ccccc34)cc2)cc1] 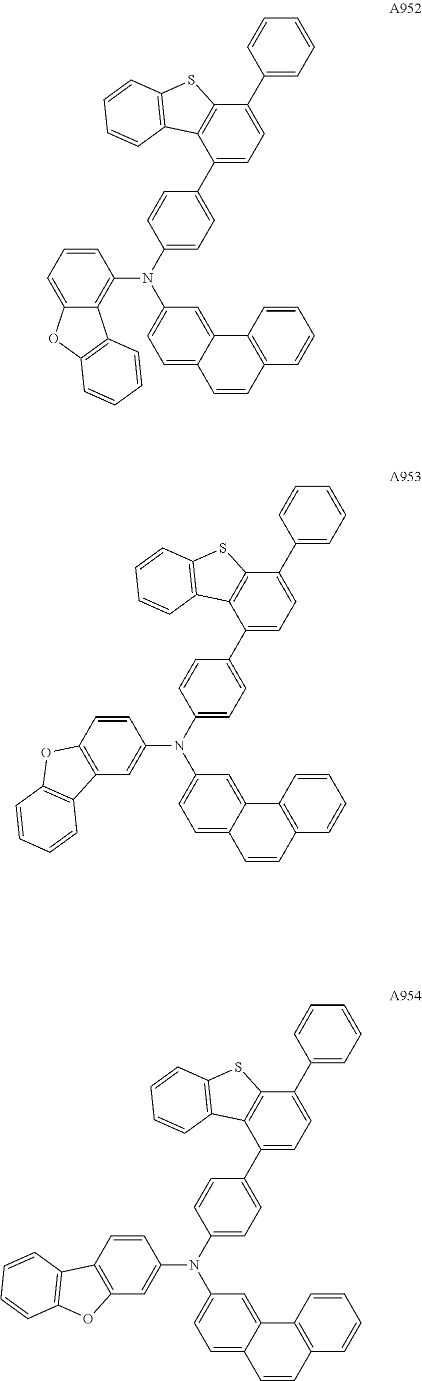 [SMILES: c1ccc(-c2ccc(-c3ccc(N(c4ccc5ccc6ccccc6c5c4)c4cccc5oc6ccccc6c45)cc3)c3c2sc2ccccc23)cc1 ; c1ccc(-c2ccc(-c3ccc(N(c4ccc5ccc6ccccc6c5c4)c4ccc5oc6ccccc6c5c4)cc3)c3c2sc2ccccc23)cc1 ; c1ccc(-c2ccc(-c3ccc(N(c4ccc5c(c4)oc4ccccc45)c4ccc5ccc6ccccc6c5c4)cc3)c3c2sc2ccccc23)cc1] 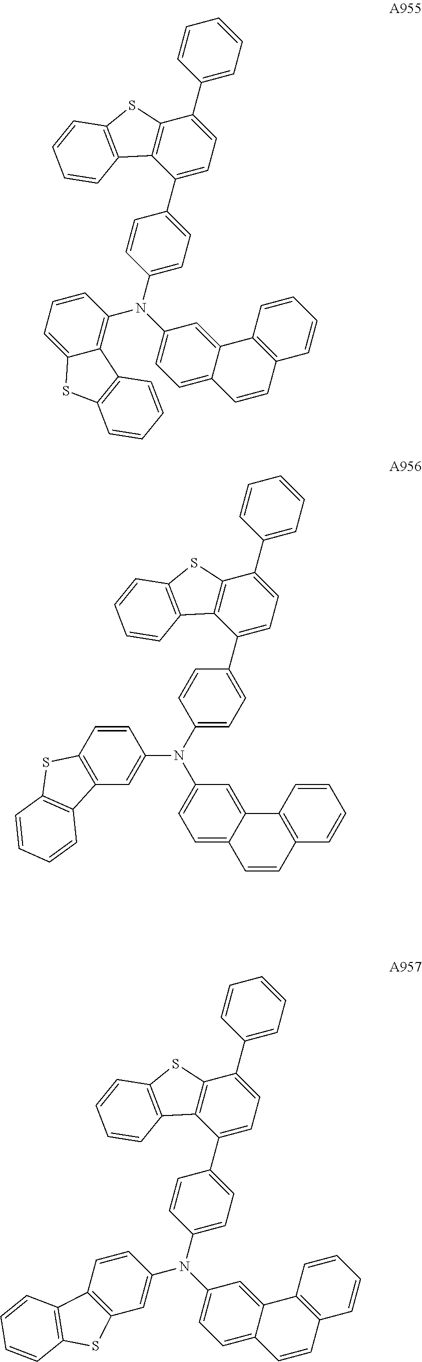 [SMILES: c1ccc(-c2ccc(-c3ccc(N(c4ccc5ccc6ccccc6c5c4)c4cccc5sc6ccccc6c45)cc3)c3c2sc2ccccc23)cc1 ; c1ccc(-c2ccc(-c3ccc(N(c4ccc5ccc6ccccc6c5c4)c4ccc5sc6ccccc6c5c4)cc3)c3c2sc2ccccc23)cc1 ; c1ccc(-c2ccc(-c3ccc(N(c4ccc5c(c4)sc4ccccc45)c4ccc5ccc6ccccc6c5c4)cc3)c3c2sc2ccccc23)cc1] 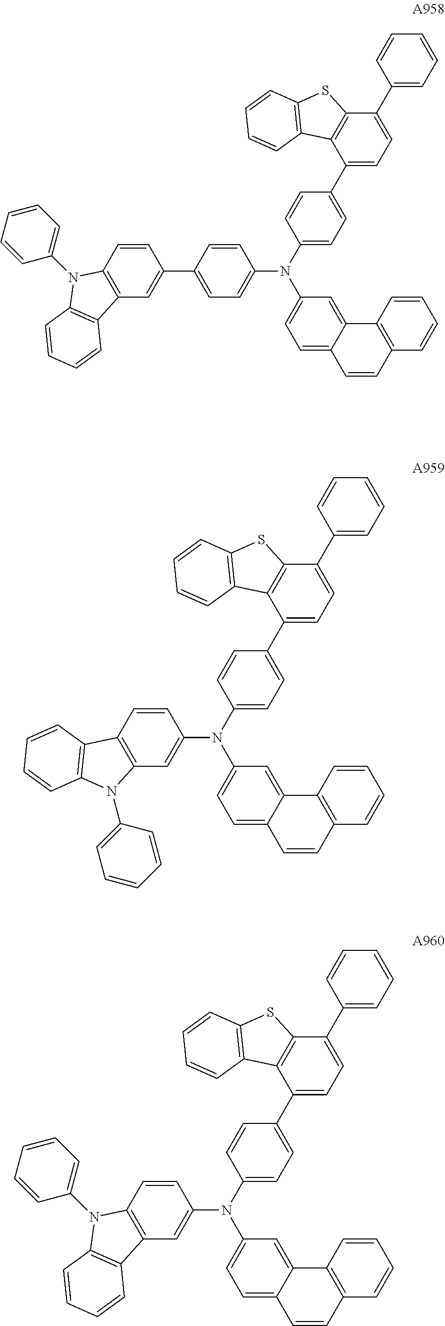 [SMILES: c1ccc(-c2ccc(-c3ccc(N(c4ccc(-c5ccc6c(c5)c5ccccc5n6-c5ccccc5)cc4)c4ccc5ccc6ccccc6c5c4)cc3)c3c2sc2ccccc23)cc1 ; c1ccc(-c2ccc(-c3ccc(N(c4ccc5ccc6ccccc6c5c4)c4ccc5c6ccccc6n(-c6ccccc6)c5c4)cc3)c3c2sc2ccccc23)cc1 ; c1ccc(-c2ccc(-c3ccc(N(c4ccc5ccc6ccccc6c5c4)c4ccc5c(c4)c4ccccc4n5-c4ccccc4)cc3)c3c2sc2ccccc23)cc1] 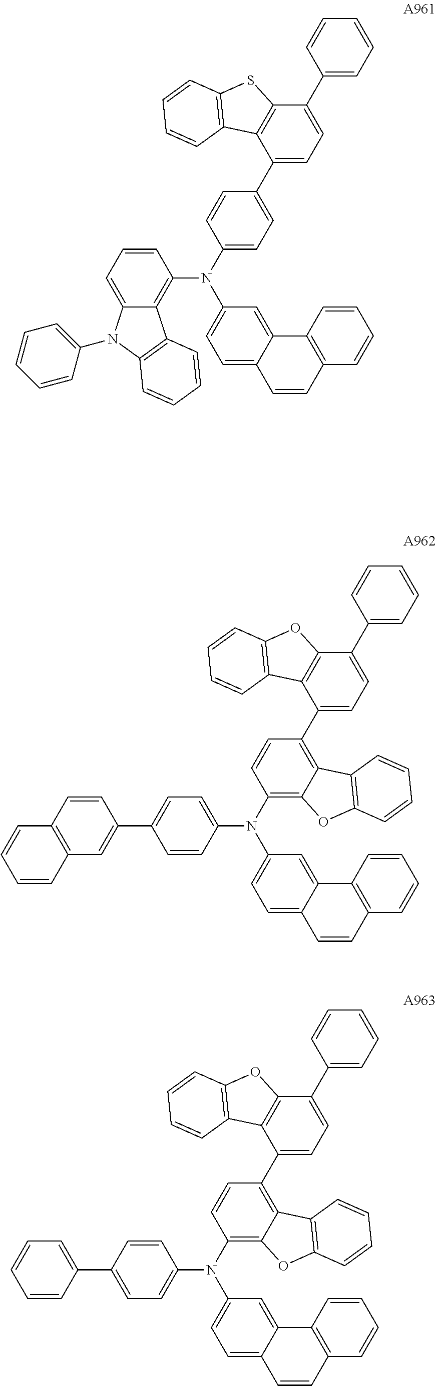 [SMILES: c1ccc(-c2ccc(-c3ccc(N(c4ccc5ccc6ccccc6c5c4)c4cccc5c4c4ccccc4n5-c4ccccc4)cc3)c3c2sc2ccccc23)cc1 ; c1ccc(-c2ccc(-c3ccc(N(c4ccc(-c5ccc6ccccc6c5)cc4)c4ccc5ccc6ccccc6c5c4)c4oc5ccccc5c34)c3c2oc2ccccc23)cc1 ; c1ccc(-c2ccc(N(c3ccc4ccc5ccccc5c4c3)c3ccc(-c4ccc(-c5ccccc5)c5oc6ccccc6c45)c4c3oc3ccccc34)cc2)cc1] 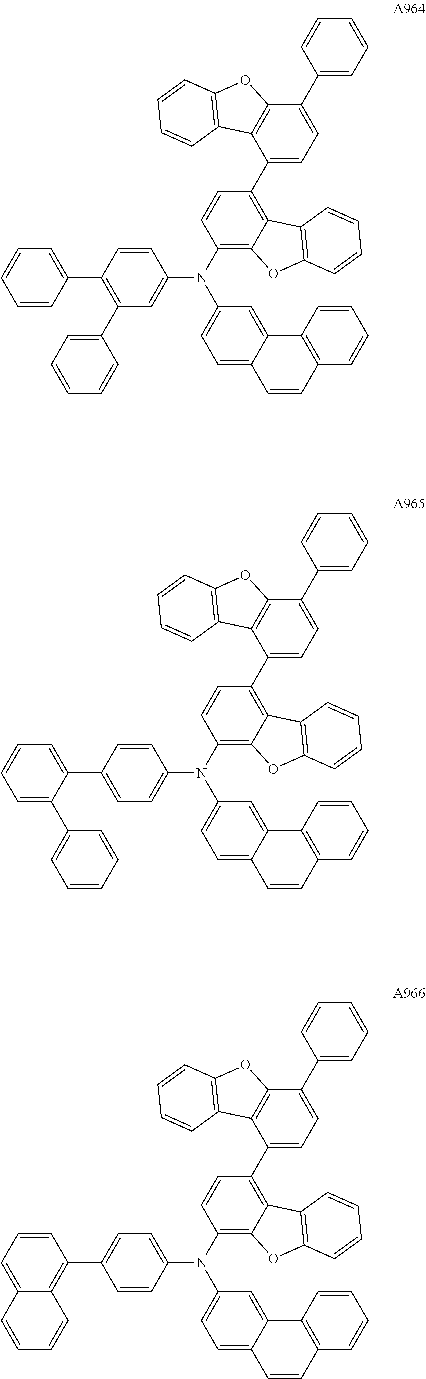 [SMILES: c1ccc(-c2ccc(N(c3ccc4ccc5ccccc5c4c3)c3ccc(-c4ccc(-c5ccccc5)c5oc6ccccc6c45)c4c3oc3ccccc34)cc2-c2ccccc2)cc1 ; c1ccc(-c2ccccc2-c2ccc(N(c3ccc4ccc5ccccc5c4c3)c3ccc(-c4ccc(-c5ccccc5)c5oc6ccccc6c45)c4c3oc3ccccc34)cc2)cc1 ; c1ccc(-c2ccc(-c3ccc(N(c4ccc(-c5cccc6ccccc56)cc4)c4ccc5ccc6ccccc6c5c4)c4oc5ccccc5c34)c3c2oc2ccccc23)cc1] 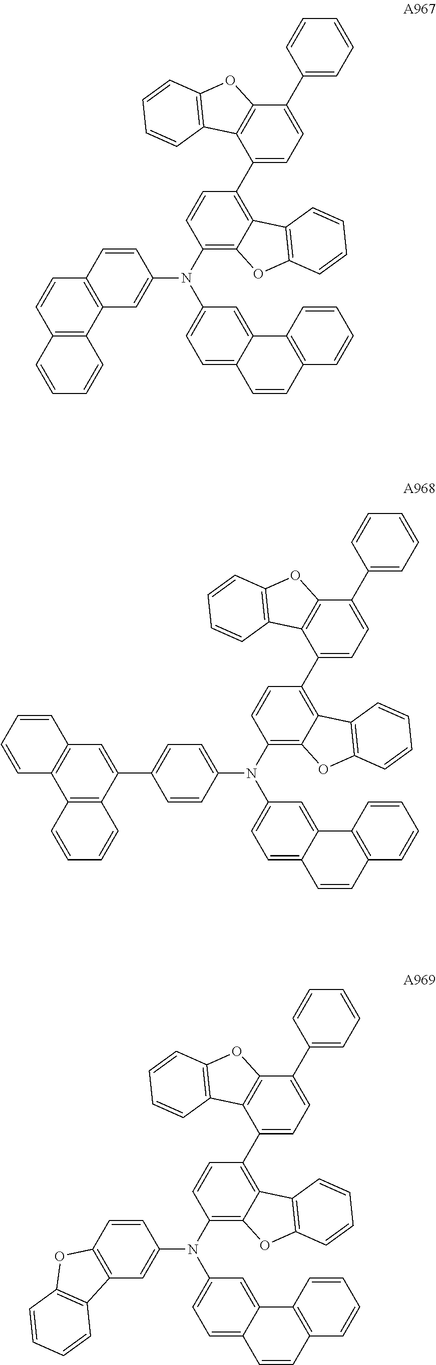 [SMILES: c1ccc(-c2ccc(-c3ccc(N(c4ccc5ccc6ccccc6c5c4)c4ccc5ccc6ccccc6c5c4)c4oc5ccccc5c34)c3c2oc2ccccc23)cc1 ; c1ccc(-c2ccc(-c3ccc(N(c4ccc(-c5cc6ccccc6c6ccccc56)cc4)c4ccc5ccc6ccccc6c5c4)c4oc5ccccc5c34)c3c2oc2ccccc23)cc1 ; c1ccc(-c2ccc(-c3ccc(N(c4ccc5ccc6ccccc6c5c4)c4ccc5oc6ccccc6c5c4)c4oc5ccccc5c34)c3c2oc2ccccc23)cc1] 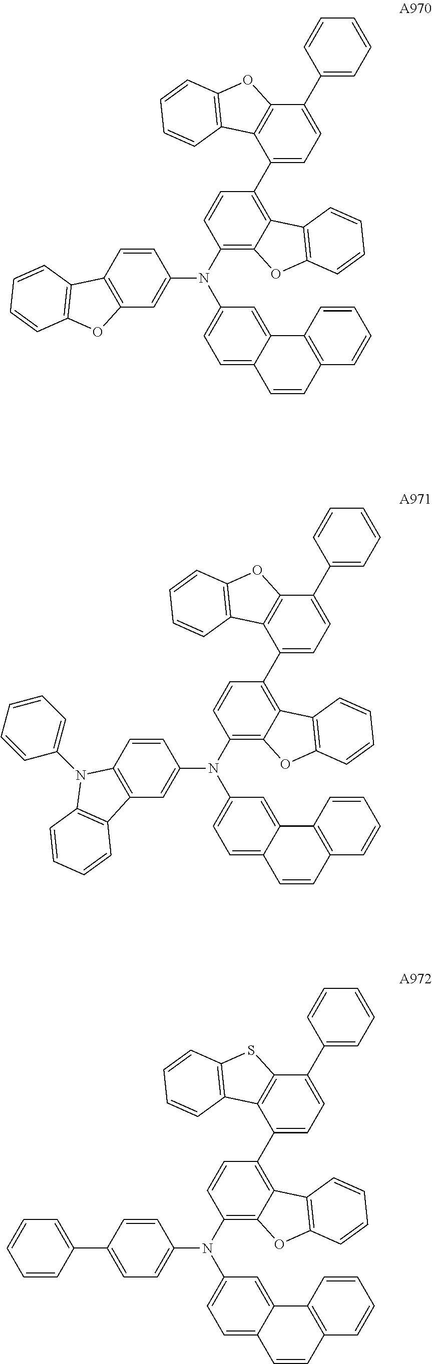 [SMILES: c1ccc(-c2ccc(-c3ccc(N(c4ccc5c(c4)oc4ccccc45)c4ccc5ccc6ccccc6c5c4)c4oc5ccccc5c34)c3c2oc2ccccc23)cc1 ; c1ccc(-c2ccc(-c3ccc(N(c4ccc5ccc6ccccc6c5c4)c4ccc5c(c4)c4ccccc4n5-c4ccccc4)c4oc5ccccc5c34)c3c2oc2ccccc23)cc1 ; c1ccc(-c2ccc(N(c3ccc4ccc5ccccc5c4c3)c3ccc(-c4ccc(-c5ccccc5)c5sc6ccccc6c45)c4c3oc3ccccc34)cc2)cc1] 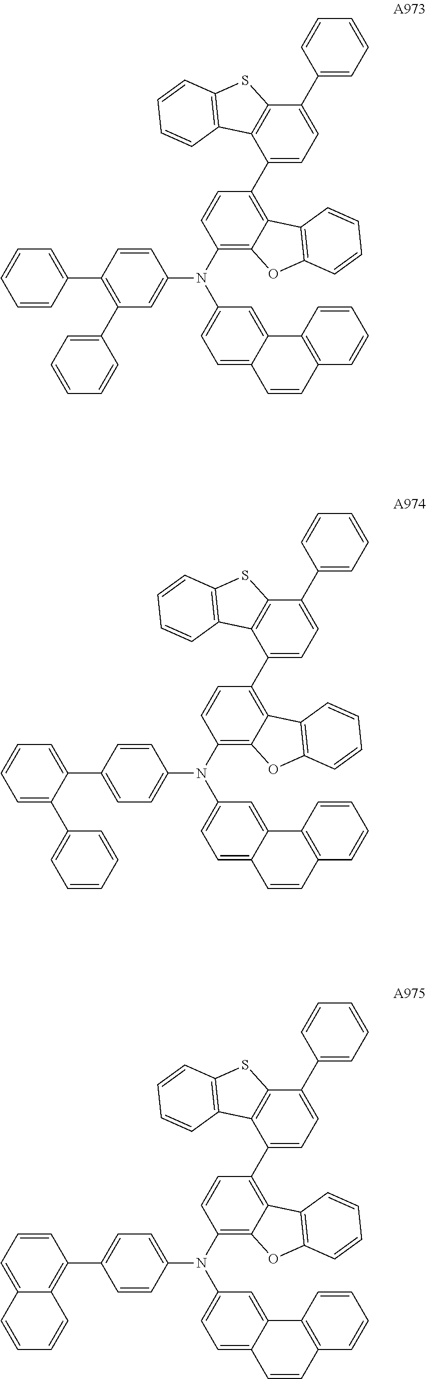 [SMILES: c1ccc(-c2ccc(N(c3ccc4ccc5ccccc5c4c3)c3ccc(-c4ccc(-c5ccccc5)c5sc6ccccc6c45)c4c3oc3ccccc34)cc2-c2ccccc2)cc1 ; c1ccc(-c2ccccc2-c2ccc(N(c3ccc4ccc5ccccc5c4c3)c3ccc(-c4ccc(-c5ccccc5)c5sc6ccccc6c45)c4c3oc3ccccc34)cc2)cc1 ; c1ccc(-c2ccc(-c3ccc(N(c4ccc(-c5cccc6ccccc56)cc4)c4ccc5ccc6ccccc6c5c4)c4oc5ccccc5c34)c3c2sc2ccccc23)cc1] 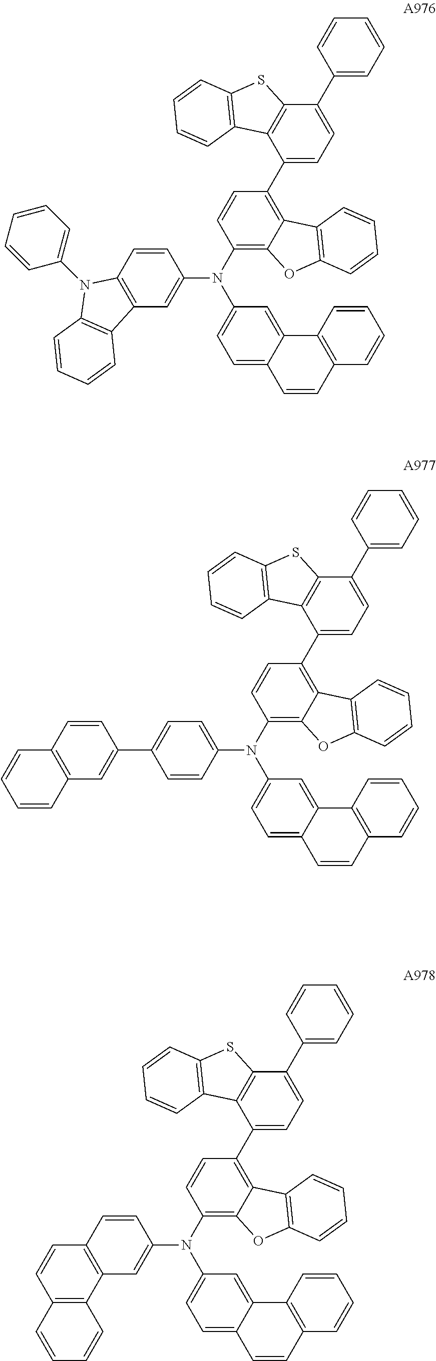 [SMILES: c1ccc(-c2ccc(-c3ccc(N(c4ccc5ccc6ccccc6c5c4)c4ccc5c(c4)c4ccccc4n5-c4ccccc4)c4oc5ccccc5c34)c3c2sc2ccccc23)cc1 ; c1ccc(-c2ccc(-c3ccc(N(c4ccc(-c5ccc6ccccc6c5)cc4)c4ccc5ccc6ccccc6c5c4)c4oc5ccccc5c34)c3c2sc2ccccc23)cc1 ; c1ccc(-c2ccc(-c3ccc(N(c4ccc5ccc6ccccc6c5c4)c4ccc5ccc6ccccc6c5c4)c4oc5ccccc5c34)c3c2sc2ccccc23)cc1] 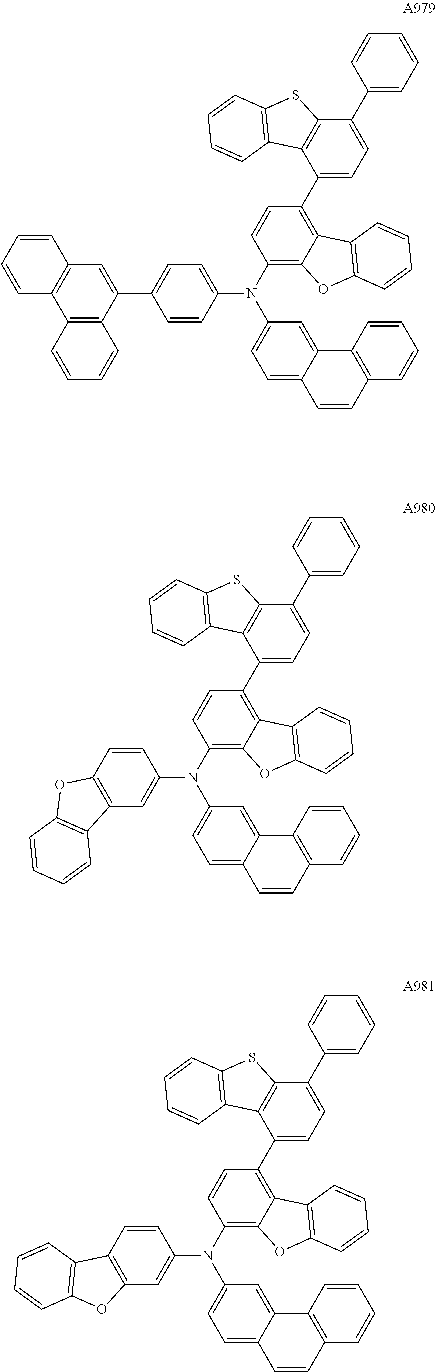 [SMILES: c1ccc(-c2ccc(-c3ccc(N(c4ccc(-c5cc6ccccc6c6ccccc56)cc4)c4ccc5ccc6ccccc6c5c4)c4oc5ccccc5c34)c3c2sc2ccccc23)cc1 ; c1ccc(-c2ccc(-c3ccc(N(c4ccc5ccc6ccccc6c5c4)c4ccc5oc6ccccc6c5c4)c4oc5ccccc5c34)c3c2sc2ccccc23)cc1 ; c1ccc(-c2ccc(-c3ccc(N(c4ccc5c(c4)oc4ccccc45)c4ccc5ccc6ccccc6c5c4)c4oc5ccccc5c34)c3c2sc2ccccc23)cc1] 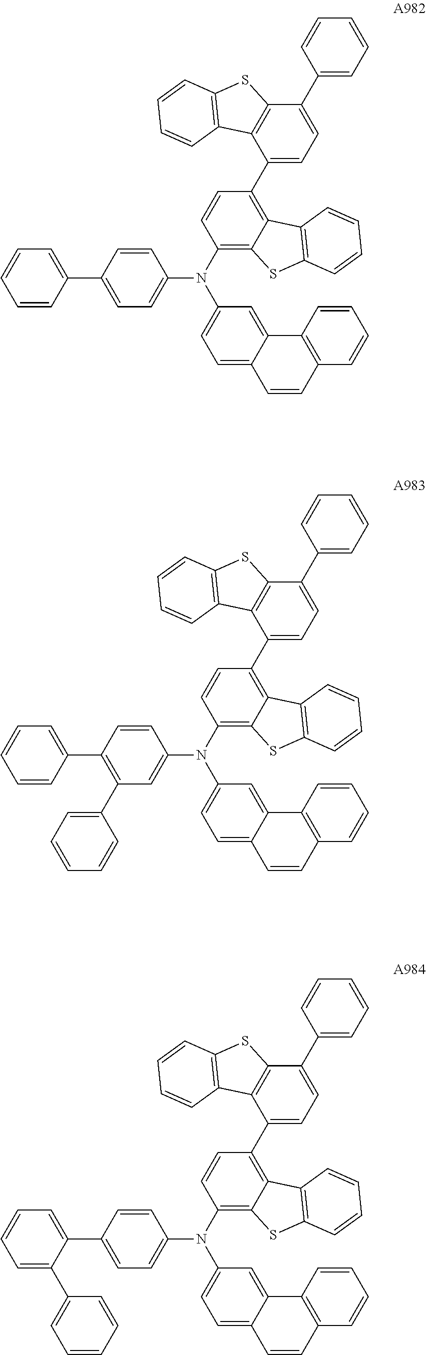 [SMILES: c1ccc(-c2ccc(N(c3ccc4ccc5ccccc5c4c3)c3ccc(-c4ccc(-c5ccccc5)c5sc6ccccc6c45)c4c3sc3ccccc34)cc2)cc1 ; c1ccc(-c2ccc(N(c3ccc4ccc5ccccc5c4c3)c3ccc(-c4ccc(-c5ccccc5)c5sc6ccccc6c45)c4c3sc3ccccc34)cc2-c2ccccc2)cc1 ; c1ccc(-c2ccccc2-c2ccc(N(c3ccc4ccc5ccccc5c4c3)c3ccc(-c4ccc(-c5ccccc5)c5sc6ccccc6c45)c4c3sc3ccccc34)cc2)cc1] 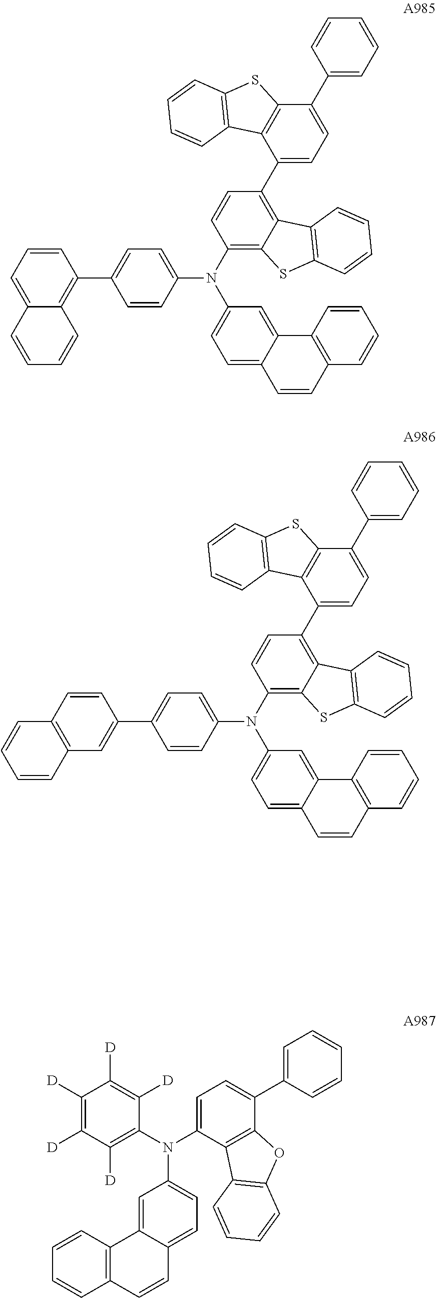 [SMILES: c1ccc(-c2ccc(-c3ccc(N(c4ccc(-c5cccc6ccccc56)cc4)c4ccc5ccc6ccccc6c5c4)c4sc5ccccc5c34)c3c2sc2ccccc23)cc1 ; c1ccc(-c2ccc(-c3ccc(N(c4ccc(-c5ccc6ccccc6c5)cc4)c4ccc5ccc6ccccc6c5c4)c4sc5ccccc5c34)c3c2sc2ccccc23)cc1 ; [2H]c1c([2H])c([2H])c(N(c2ccc3ccc4ccccc4c3c2)c2ccc(-c3ccccc3)c3oc4ccccc4c23)c([2H])c1[2H]] 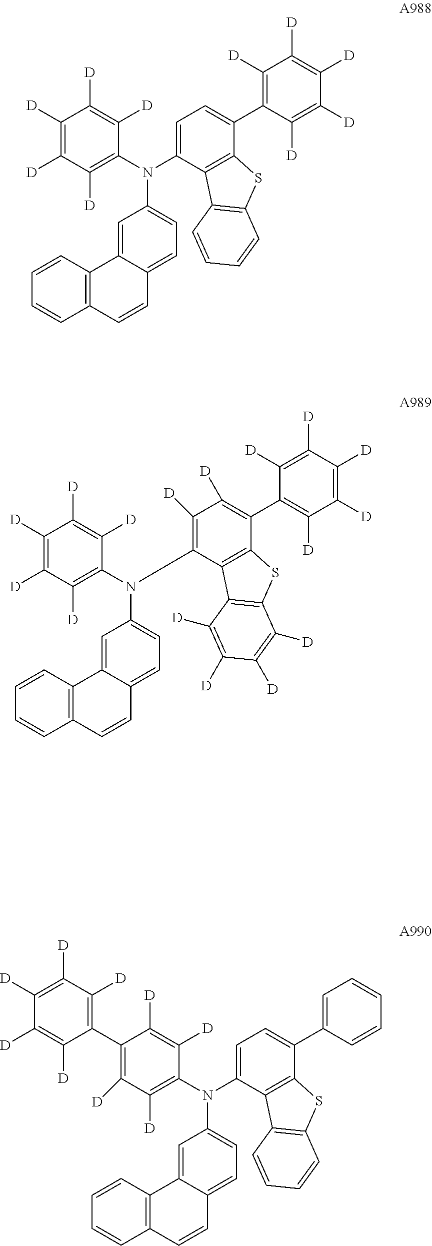 [SMILES: [2H]c1c([2H])c([2H])c(-c2ccc(N(c3ccc4ccc5ccccc5c4c3)c3c([2H])c([2H])c([2H])c([2H])c3[2H])c3c2sc2ccccc23)c([2H])c1[2H] ; [2H]c1c([2H])c([2H])c(-c2c([2H])c([2H])c(N(c3ccc4ccc5ccccc5c4c3)c3c([2H])c([2H])c([2H])c([2H])c3[2H])c3c2sc2c([2H])c([2H])c([2H])c([2H])c23)c([2H])c1[2H] ; [2H]c1c([2H])c([2H])c(-c2c([2H])c([2H])c(N(c3ccc4ccc5ccccc5c4c3)c3ccc(-c4ccccc4)c4sc5ccccc5c34)c([2H])c2[2H])c([2H])c1[2H]] 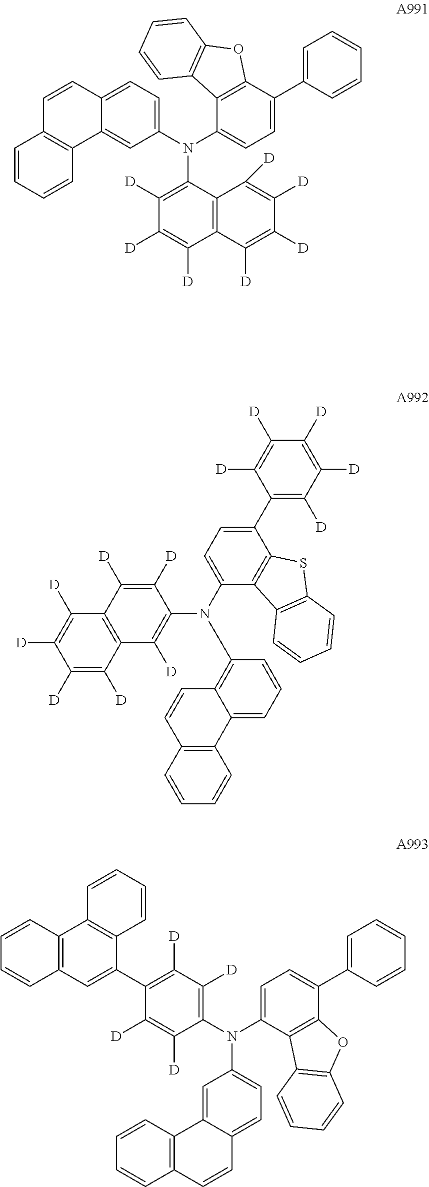 [SMILES: [2H]c1c([2H])c([2H])c2c(N(c3ccc4ccc5ccccc5c4c3)c3ccc(-c4ccccc4)c4oc5ccccc5c34)c([2H])c([2H])c([2H])c2c1[2H] ; [2H]c1c([2H])c([2H])c(-c2ccc(N(c3c([2H])c([2H])c4c([2H])c([2H])c([2H])c([2H])c4c3[2H])c3cccc4c3ccc3ccccc34)c3c2sc2ccccc23)c([2H])c1[2H] ; [2H]c1c([2H])c(N(c2ccc3ccc4ccccc4c3c2)c2ccc(-c3ccccc3)c3oc4ccccc4c23)c([2H])c([2H])c1-c1cc2ccccc2c2ccccc12] 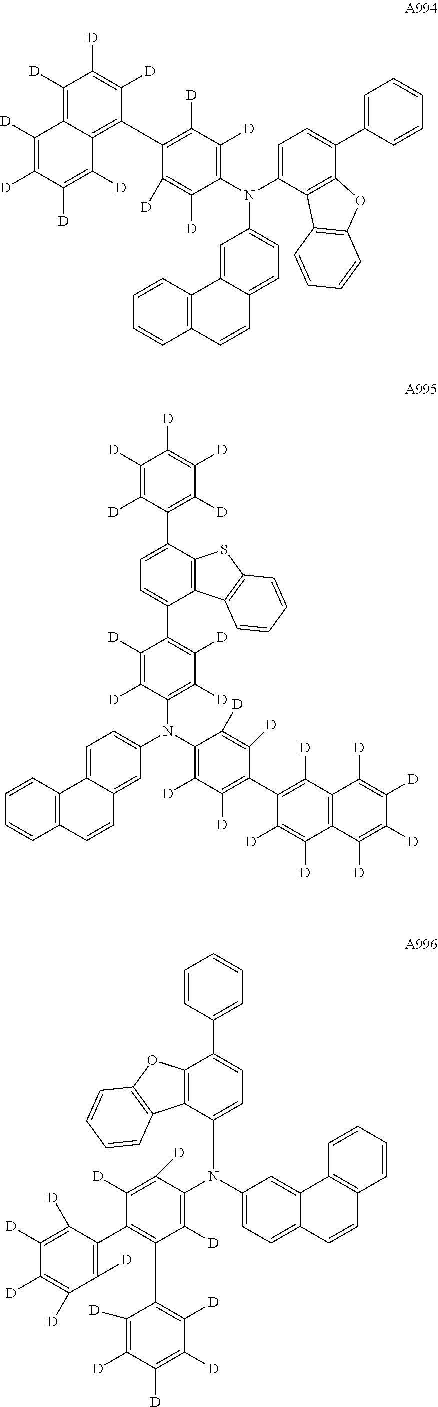 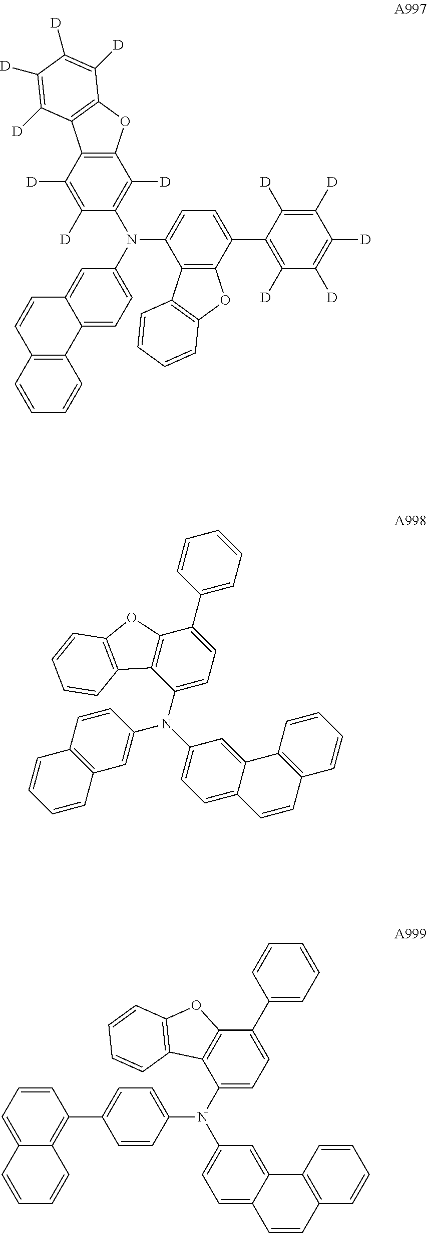 [SMILES: [2H]c1c([2H])c([2H])c(-c2ccc(N(c3ccc4c(ccc5ccccc54)c3)c3c([2H])c([2H])c4c(oc5c([2H])c([2H])c([2H])c([2H])c54)c3[2H])c3c2oc2ccccc23)c([2H])c1[2H] ; c1ccc(-c2ccc(N(c3ccc4ccccc4c3)c3ccc4ccc5ccccc5c4c3)c3c2oc2ccccc23)cc1 ; c1ccc(-c2ccc(N(c3ccc(-c4cccc5ccccc45)cc3)c3ccc4ccc5ccccc5c4c3)c3c2oc2ccccc23)cc1] 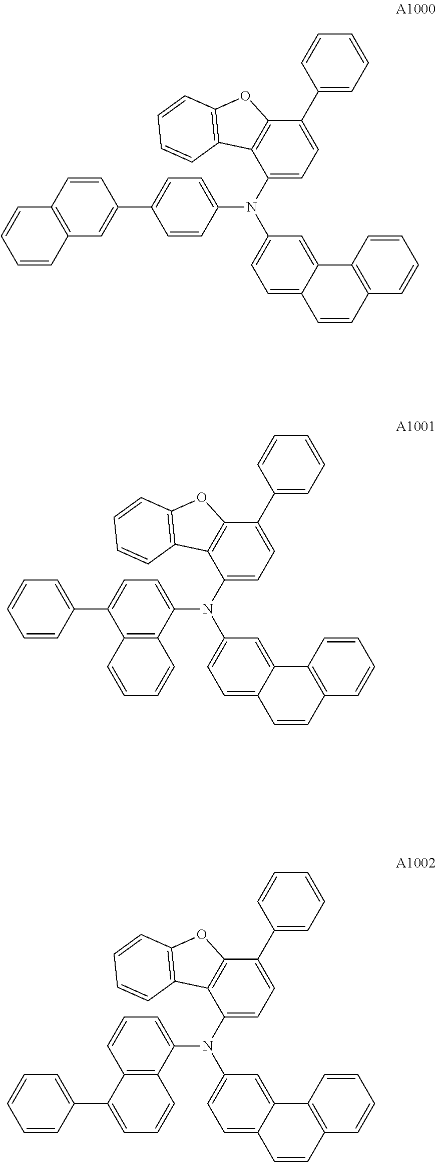 [SMILES: c1ccc(-c2ccc(N(c3ccc(-c4ccc5ccccc5c4)cc3)c3ccc4ccc5ccccc5c4c3)c3c2oc2ccccc23)cc1 ; c1ccc(-c2ccc(N(c3ccc4ccc5ccccc5c4c3)c3ccc(-c4ccccc4)c4oc5ccccc5c34)c3ccccc23)cc1 ; c1ccc(-c2cccc3c(N(c4ccc5ccc6ccccc6c5c4)c4ccc(-c5ccccc5)c5oc6ccccc6c45)cccc23)cc1] 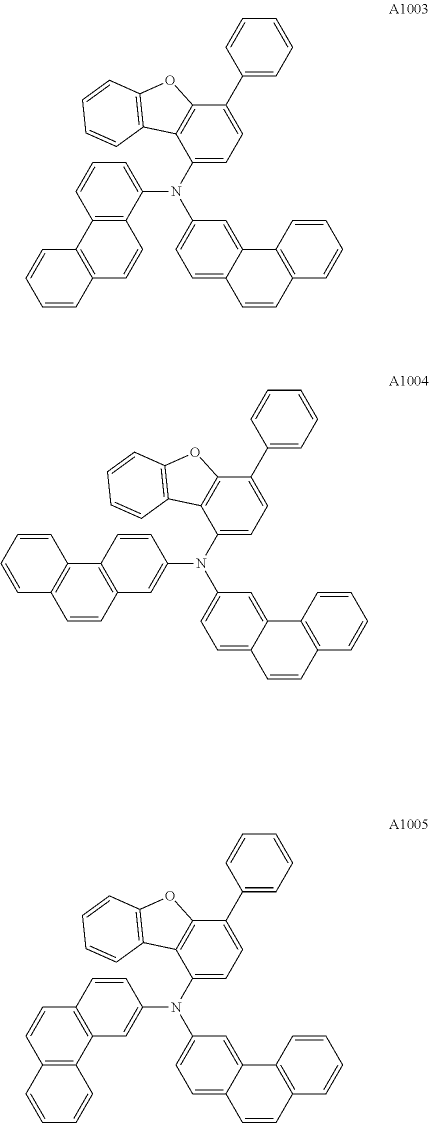 [SMILES: c1ccc(-c2ccc(N(c3ccc4ccc5ccccc5c4c3)c3cccc4c3ccc3ccccc34)c3c2oc2ccccc23)cc1 ; c1ccc(-c2ccc(N(c3ccc4c(ccc5ccccc54)c3)c3ccc4ccc5ccccc5c4c3)c3c2oc2ccccc23)cc1 ; c1ccc(-c2ccc(N(c3ccc4ccc5ccccc5c4c3)c3ccc4ccc5ccccc5c4c3)c3c2oc2ccccc23)cc1] 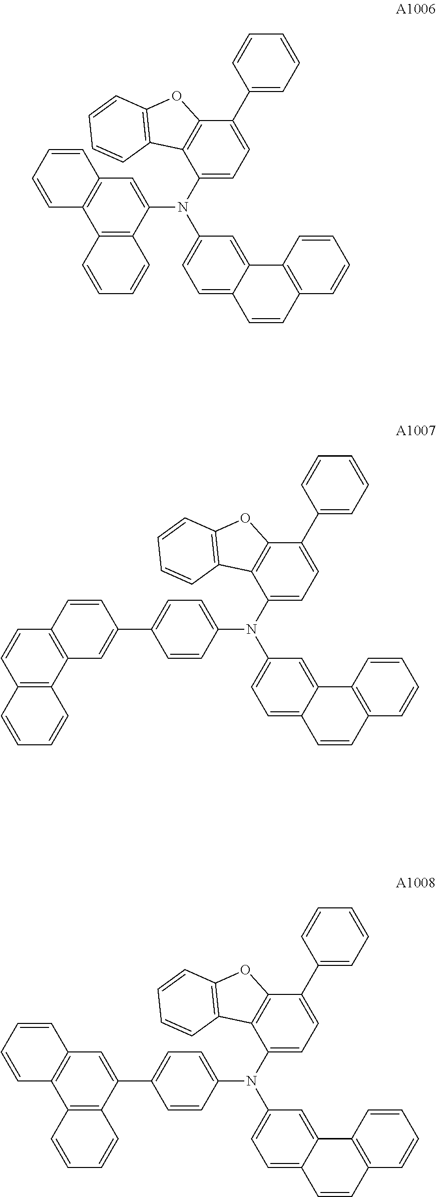 [SMILES: c1ccc(-c2ccc(N(c3ccc4ccc5ccccc5c4c3)c3cc4ccccc4c4ccccc34)c3c2oc2ccccc23)cc1 ; c1ccc(-c2ccc(N(c3ccc(-c4ccc5ccc6ccccc6c5c4)cc3)c3ccc4ccc5ccccc5c4c3)c3c2oc2ccccc23)cc1 ; c1ccc(-c2ccc(N(c3ccc(-c4cc5ccccc5c5ccccc45)cc3)c3ccc4ccc5ccccc5c4c3)c3c2oc2ccccc23)cc1] 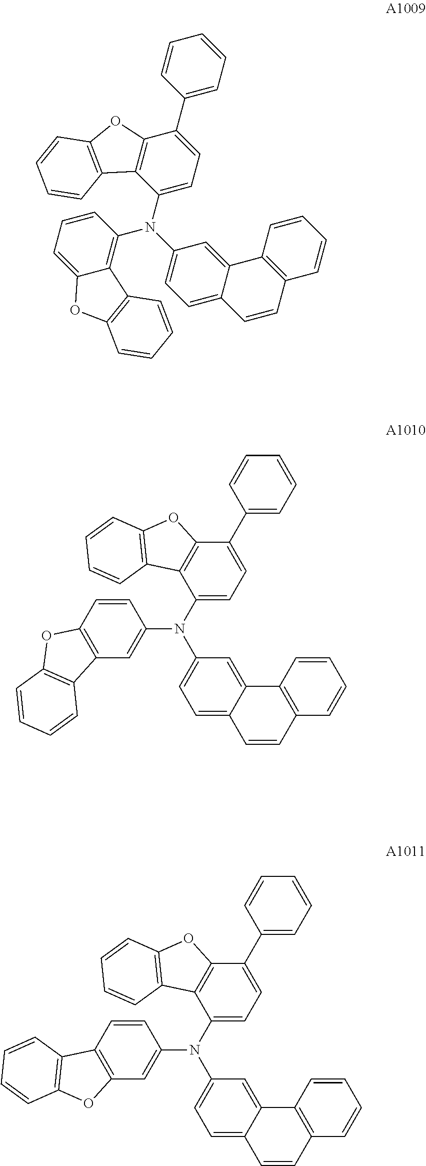 [SMILES: c1ccc(-c2ccc(N(c3ccc4ccc5ccccc5c4c3)c3cccc4oc5ccccc5c34)c3c2oc2ccccc23)cc1 ; c1ccc(-c2ccc(N(c3ccc4ccc5ccccc5c4c3)c3ccc4oc5ccccc5c4c3)c3c2oc2ccccc23)cc1 ; c1ccc(-c2ccc(N(c3ccc4c(c3)oc3ccccc34)c3ccc4ccc5ccccc5c4c3)c3c2oc2ccccc23)cc1] 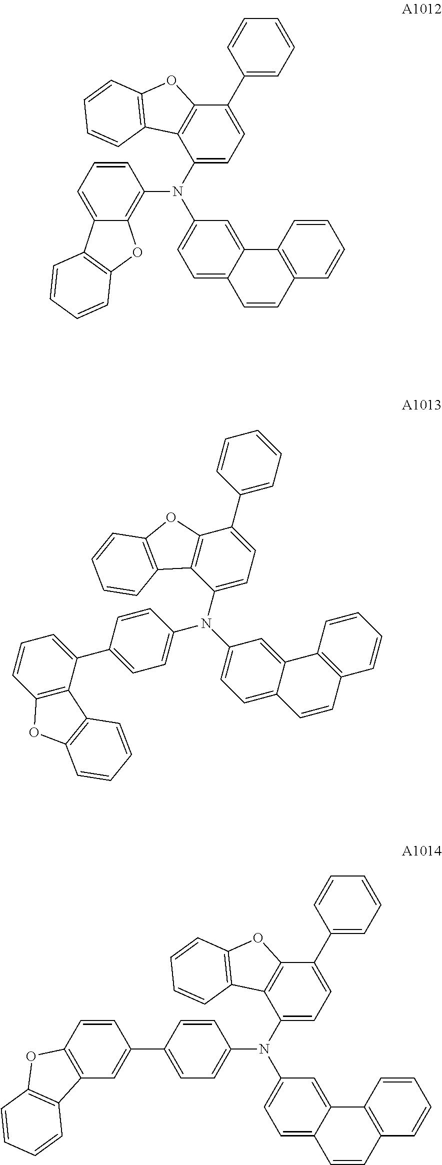 [SMILES: c1ccc(-c2ccc(N(c3ccc4ccc5ccccc5c4c3)c3cccc4c3oc3ccccc34)c3c2oc2ccccc23)cc1 ; c1ccc(-c2ccc(N(c3ccc(-c4cccc5oc6ccccc6c45)cc3)c3ccc4ccc5ccccc5c4c3)c3c2oc2ccccc23)cc1 ; c1ccc(-c2ccc(N(c3ccc(-c4ccc5oc6ccccc6c5c4)cc3)c3ccc4ccc5ccccc5c4c3)c3c2oc2ccccc23)cc1] 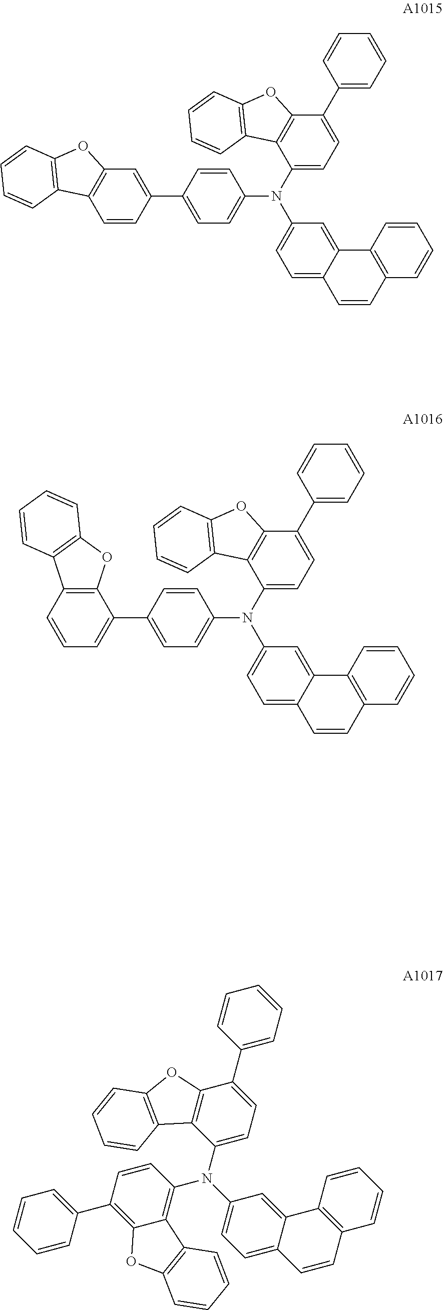 [SMILES: c1ccc(-c2ccc(N(c3ccc(-c4ccc5c(c4)oc4ccccc45)cc3)c3ccc4ccc5ccccc5c4c3)c3c2oc2ccccc23)cc1 ; c1ccc(-c2ccc(N(c3ccc(-c4cccc5c4oc4ccccc45)cc3)c3ccc4ccc5ccccc5c4c3)c3c2oc2ccccc23)cc1 ; c1ccc(-c2ccc(N(c3ccc4ccc5ccccc5c4c3)c3ccc(-c4ccccc4)c4oc5ccccc5c34)c3c2oc2ccccc23)cc1] 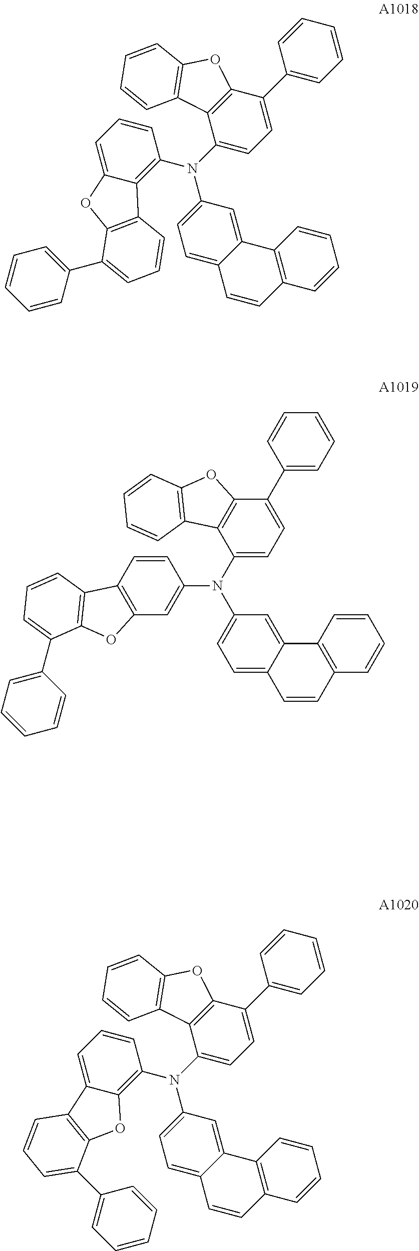 [SMILES: c1ccc(-c2cccc3c2oc2cccc(N(c4ccc5ccc6ccccc6c5c4)c4ccc(-c5ccccc5)c5oc6ccccc6c45)c23)cc1 ; c1ccc(-c2cccc3c2oc2cc(N(c4ccc5ccc6ccccc6c5c4)c4ccc(-c5ccccc5)c5oc6ccccc6c45)ccc23)cc1 ; c1ccc(-c2cccc3c2oc2c(N(c4ccc5ccc6ccccc6c5c4)c4ccc(-c5ccccc5)c5oc6ccccc6c45)cccc23)cc1] 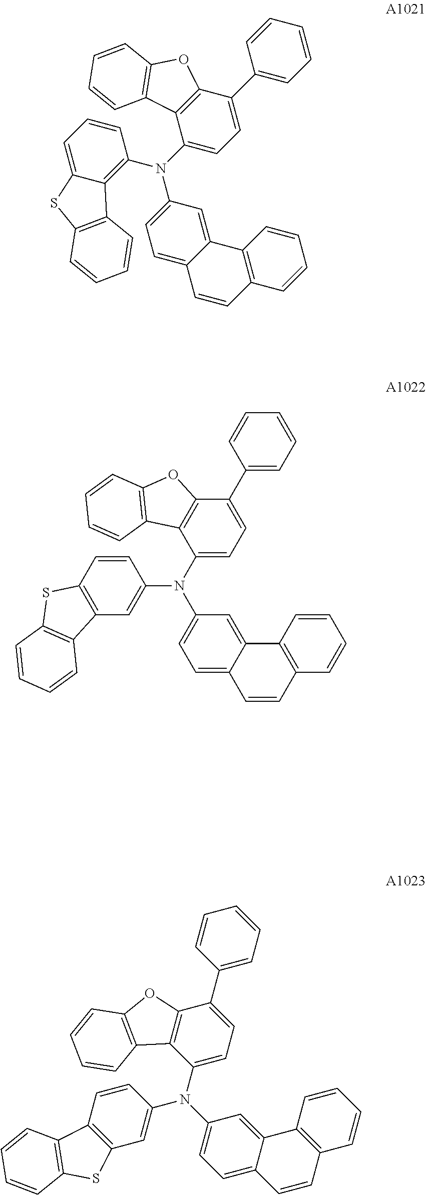 [SMILES: c1ccc(-c2ccc(N(c3ccc4ccc5ccccc5c4c3)c3cccc4sc5ccccc5c34)c3c2oc2ccccc23)cc1 ; c1ccc(-c2ccc(N(c3ccc4ccc5ccccc5c4c3)c3ccc4sc5ccccc5c4c3)c3c2oc2ccccc23)cc1 ; c1ccc(-c2ccc(N(c3ccc4c(c3)sc3ccccc34)c3ccc4ccc5ccccc5c4c3)c3c2oc2ccccc23)cc1] 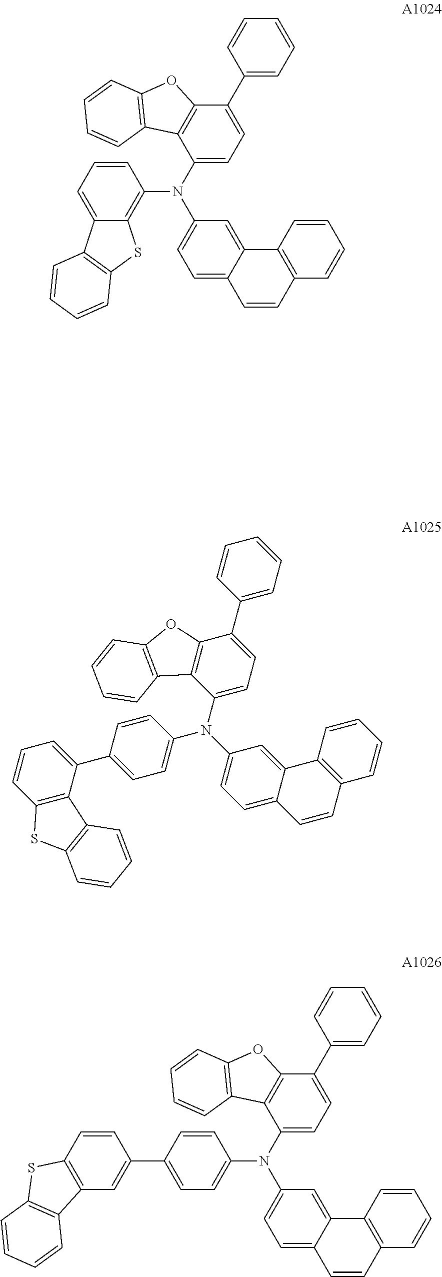 [SMILES: c1ccc(-c2ccc(N(c3ccc4ccc5ccccc5c4c3)c3cccc4c3sc3ccccc34)c3c2oc2ccccc23)cc1 ; c1ccc(-c2ccc(N(c3ccc(-c4cccc5sc6ccccc6c45)cc3)c3ccc4ccc5ccccc5c4c3)c3c2oc2ccccc23)cc1 ; c1ccc(-c2ccc(N(c3ccc(-c4ccc5sc6ccccc6c5c4)cc3)c3ccc4ccc5ccccc5c4c3)c3c2oc2ccccc23)cc1] 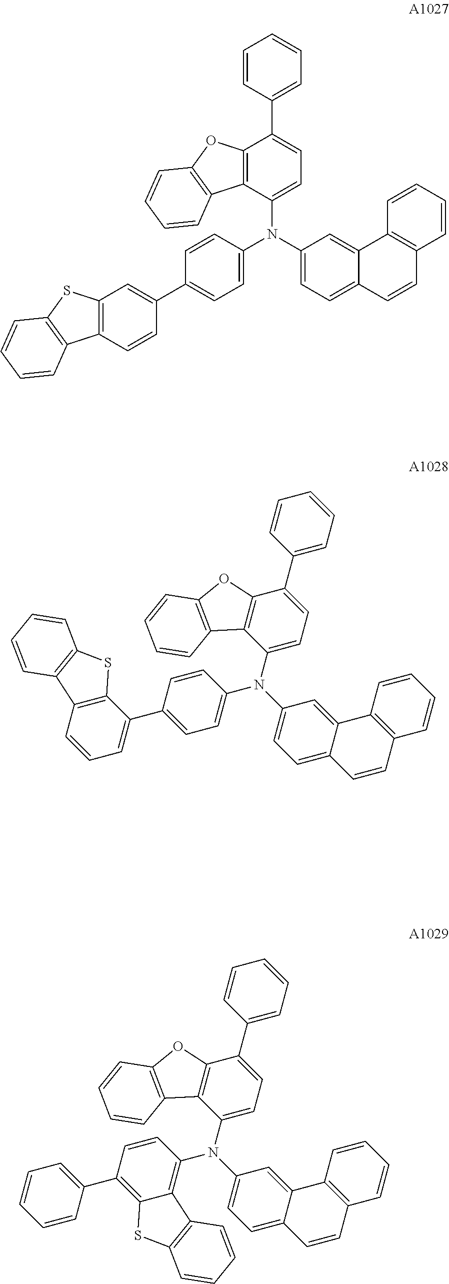 [SMILES: c1ccc(-c2ccc(N(c3ccc(-c4ccc5c(c4)sc4ccccc45)cc3)c3ccc4ccc5ccccc5c4c3)c3c2oc2ccccc23)cc1 ; c1ccc(-c2ccc(N(c3ccc(-c4cccc5c4sc4ccccc45)cc3)c3ccc4ccc5ccccc5c4c3)c3c2oc2ccccc23)cc1 ; c1ccc(-c2ccc(N(c3ccc4ccc5ccccc5c4c3)c3ccc(-c4ccccc4)c4sc5ccccc5c34)c3c2oc2ccccc23)cc1] 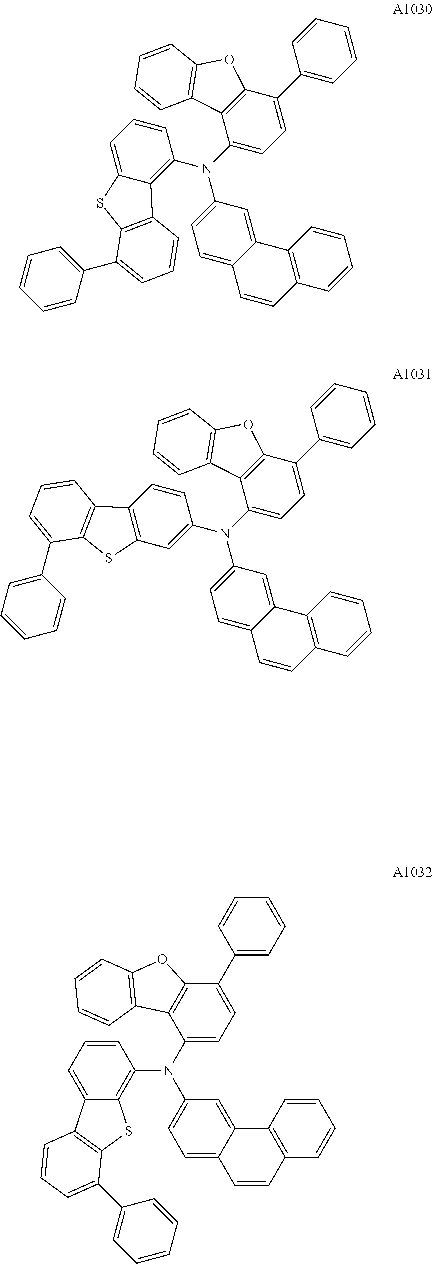 [SMILES: c1ccc(-c2ccc(N(c3ccc4ccc5ccccc5c4c3)c3cccc4sc5c(-c6ccccc6)cccc5c34)c3c2oc2ccccc23)cc1 ; c1ccc(-c2ccc(N(c3ccc4c(c3)sc3c(-c5ccccc5)cccc34)c3ccc4ccc5ccccc5c4c3)c3c2oc2ccccc23)cc1 ; c1ccc(-c2ccc(N(c3ccc4ccc5ccccc5c4c3)c3cccc4c3sc3c(-c5ccccc5)cccc34)c3c2oc2ccccc23)cc1] 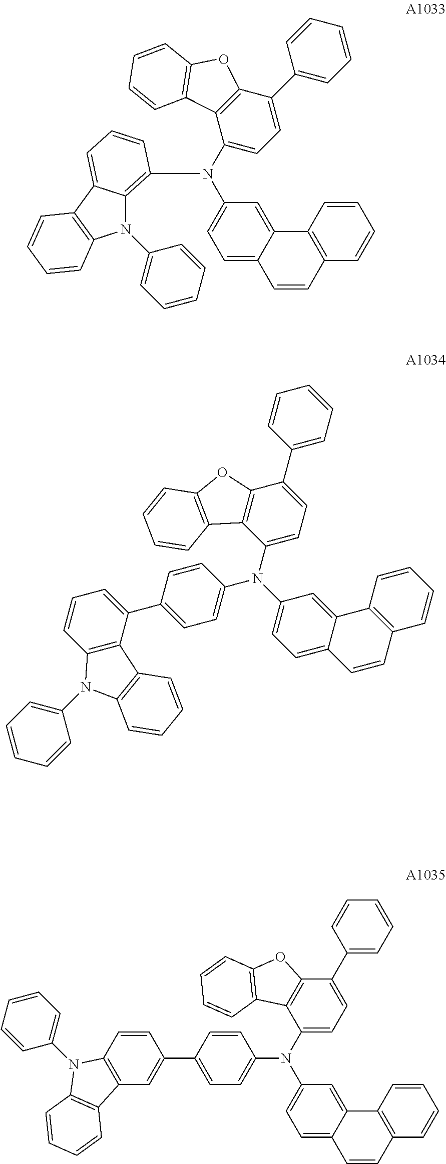 [SMILES: c1ccc(-c2ccc(N(c3ccc4ccc5ccccc5c4c3)c3cccc4c5ccccc5n(-c5ccccc5)c34)c3c2oc2ccccc23)cc1 ; c1ccc(-c2ccc(N(c3ccc(-c4cccc5c4c4ccccc4n5-c4ccccc4)cc3)c3ccc4ccc5ccccc5c4c3)c3c2oc2ccccc23)cc1 ; c1ccc(-c2ccc(N(c3ccc(-c4ccc5c(c4)c4ccccc4n5-c4ccccc4)cc3)c3ccc4ccc5ccccc5c4c3)c3c2oc2ccccc23)cc1] 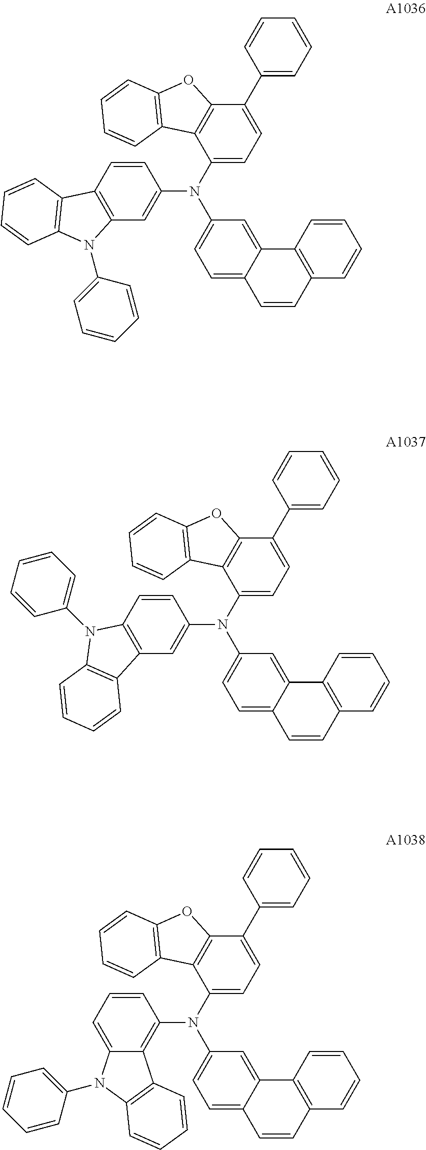 [SMILES: c1ccc(-c2ccc(N(c3ccc4ccc5ccccc5c4c3)c3ccc4c5ccccc5n(-c5ccccc5)c4c3)c3c2oc2ccccc23)cc1 ; c1ccc(-c2ccc(N(c3ccc4ccc5ccccc5c4c3)c3ccc4c(c3)c3ccccc3n4-c3ccccc3)c3c2oc2ccccc23)cc1 ; c1ccc(-c2ccc(N(c3ccc4ccc5ccccc5c4c3)c3cccc4c3c3ccccc3n4-c3ccccc3)c3c2oc2ccccc23)cc1] 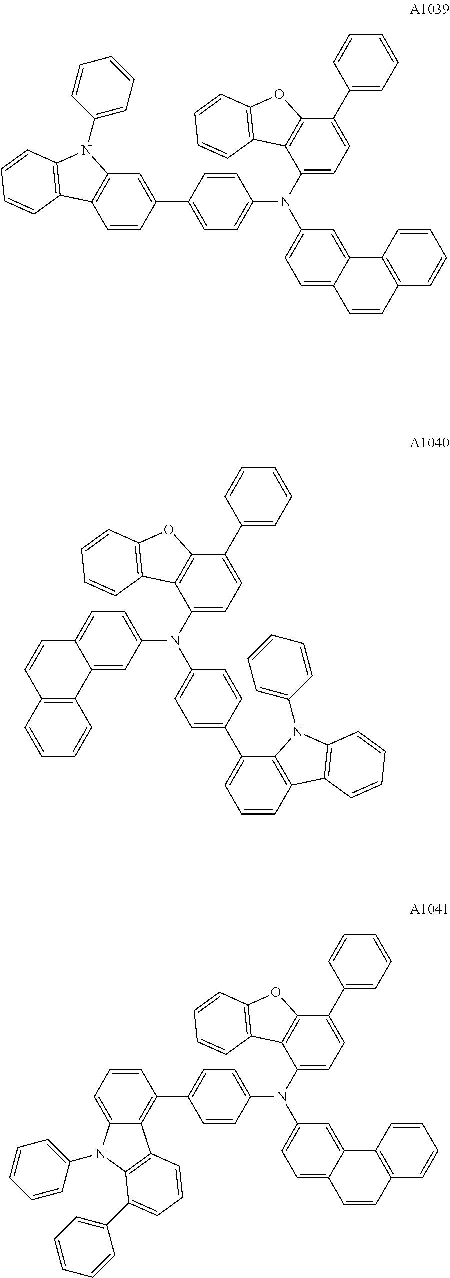 [SMILES: c1ccc(-c2ccc(N(c3ccc(-c4ccc5c6ccccc6n(-c6ccccc6)c5c4)cc3)c3ccc4ccc5ccccc5c4c3)c3c2oc2ccccc23)cc1 ; c1ccc(-c2ccc(N(c3ccc(-c4cccc5c6ccccc6n(-c6ccccc6)c45)cc3)c3ccc4ccc5ccccc5c4c3)c3c2oc2ccccc23)cc1 ; c1ccc(-c2ccc(N(c3ccc(-c4cccc5c4c4cccc(-c6ccccc6)c4n5-c4ccccc4)cc3)c3ccc4ccc5ccccc5c4c3)c3c2oc2ccccc23)cc1] 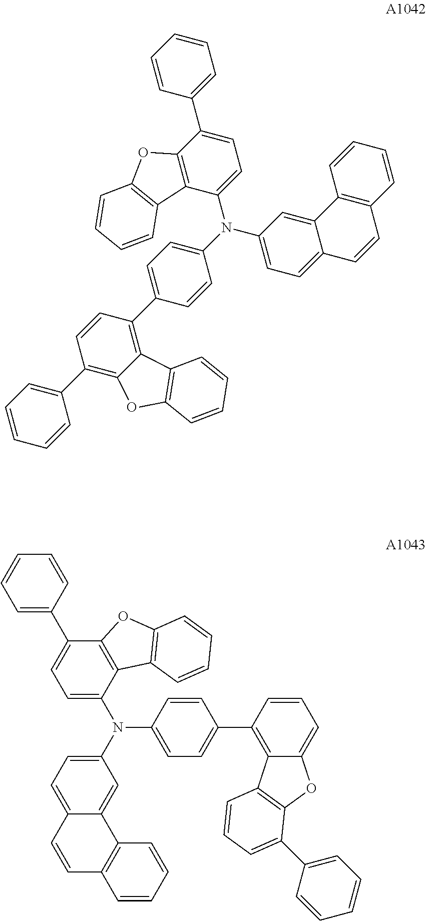 [SMILES: c1ccc(-c2ccc(-c3ccc(N(c4ccc5ccc6ccccc6c5c4)c4ccc(-c5ccccc5)c5oc6ccccc6c45)cc3)c3c2oc2ccccc23)cc1 ; c1ccc(-c2cccc3c2oc2cccc(-c4ccc(N(c5ccc6ccc7ccccc7c6c5)c5ccc(-c6ccccc6)c6oc7ccccc7c56)cc4)c23)cc1]
wherein in Compound Group 1,
D is a deuterium atom.

20. A display device comprising:
a circuit layer disposed on a base layer; and
a display element layer disposed on the circuit layer and including a light-emitting element, wherein
the light-emitting element includes:
a first electrode;
a hole transport region disposed on the first electrode;
an emission layer disposed on the hole transport region;
an electron transport region disposed on the emission layer; and
a second electrode disposed on the electron transport region, and
the hole transport region includes an amine compound represented by Formula 1: 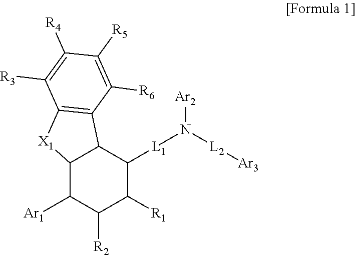
wherein Formula 1,
X₁ is O or S,
L₁ and L₂ are each independently a direct linkage, a substituted or unsubstituted arylene group having 6 to 15 ring-forming carbon atoms, or a substituted or unsubstituted heteroarylene group having 2 to 15 ring-forming carbon atoms,
Ar₁ is a substituted or unsubstituted aryl group having 6 to 15 ring-forming carbon atoms or a substituted or unsubstituted heteroaryl group having 2 to 15 ring-forming carbon atoms,
Ar₂ is a group represented by Formula 2,
Ar₃ is a substituted or unsubstituted aryl group having 6 to 30 ring-forming carbon atoms or a substituted or unsubstituted heteroaryl group having 2 to 30 ring-forming carbon atoms, and
R₁ to R₆ are each independently a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms; 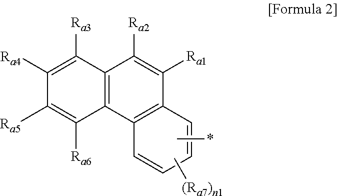
wherein in Formula 2,
n1 is an integer from 0 to 3,
Rₐ₁ to Rₐ₇ are each independently a hydrogen atom, a deuterium atom, a substituted or unsubstituted alkyl group having 1 to 10 carbon atoms, a substituted or unsubstituted aryl group having 6 to 15 ring-forming carbon atoms, or a substituted or unsubstituted heteroaryl group having 2 to 15 ring-forming carbon atoms, and [symbol]
represents a bond to a neighboring atom.
